(19) United States
(12) Patent Application Publication
(10) Pub. No.: US 2023/0265116 A1
(43) Pub. Date: Aug. 24, 2023

(54) DEGRADATION OF (EGFR) BY CONJUGATION OF EGFR INHIBITORS WITH E3 LIGASE LIGAND AND METHODS OF USE

(71) Applicant: BEIGENE, LTD., Camana Bay, Grand Cayman (KY)

(72) Inventors: Bailin LEI, Beijing (CN); Huaqing LIU, Beijing (CN); Songzhe HAN, Beijing (CN); Zhiwei WANG, Beijing (CN)

(21) Appl. No.: 18/016,292

(22) PCT Filed: Jul. 15, 2021
(86) PCT No.: PCT/CN2021/106482

§ 371 Date: Jan. 13, 2023

(30) Foreign Application Priority Data
Jul. 16, 2020 (WO) PCT/CN2020/102501
Sep. 3, 2020 (WO) PCT/CN2020/113237
Jan. 8, 2021 (WO) PCT/CN2021/070896
Jun. 21, 2021 (WO) PCT/CN2021/101275

(51) Int. Cl. C07F 9/6558; C07D 401/14; C07D 487/10

(52) U.S. Cl. CPC C07F 9/65583; C07D 401/14; C07D 487/10

(57) ABSTRACT

Disclosed herein are novel bifunctional compounds formed by conjugating EGFR inhibitor moieties with E3 ligase ligand moieties, which function to recruit targeted proteins to E3 ubiquitin ligase for degradation, and methods of preparation and uses thereof.

### FIELD OF THE INVENTION

Disclosed herein are novel bifunctional compounds formed by conjugating EGFR inhibitor moieties with E3 ligase ligand moieties, which function to recruit targeted proteins to E3 ubiquitin ligase for degradation, and methods of preparation and uses thereof.

### BACKGROUND OF THE INVENTION

Proteolysis targeting chimera (PROTAC) consists of two covalently linked protein-binding molecules: one capable of engaging an E3 ubiquitin ligase, and another that binds to the protein of interest (POI) a target meant for degradation (Sakamoto K M et al., Proc. Natl. Acad. Sci. 2001, 98: 8554-9; Sakamoto K. M. et al., Methods Enzymol. 2005; 399:833-847). Rather than inhibiting the target protein's enzymatic activity, recruitment of the E3 ligase to the specific unwanted proteins results in ubiquitination and subsequent degradation of the target protein by the proteasome. The whole process of ubiquitination and proteasomal degradation is known as the ubiquitin-proteasome pathway (UPP) (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Proteasomes are protein complexes which degrade unneeded, misfolded or abnormal proteins into small peptides to maintain health and productivity of the cells. Ubiquitin ligases, also called an E3 ubiquitin ligase, directly catalyze the transfer of ubiquitin from the E2 to the target protein for degradation. Although the human genome encodes over 600 putative E3 ligases, only a limited number of E3 ubiquitin ligases have been widely applied by small molecule PROTAC technology: cereblon (CRBN), Von Hippel-Lindau (VHL), mouse double minute 2 homologue (MDM2) and cellular inhibitor of apoptosis protein (cIAP) (Philipp O. et al., Chem. Biol. 2017, 12, 2570-2578), recombinant Human Ring Finger Protein 114 (RNF114) (Spradlin, J. N. et al. Nat. Chem. Biol. 2019, 15, 747-755) and DDB1 And CUL4 Associated Factor 16 (DCAF16) (Zhang, X. et al. Nat. Chem. Biol. 2019, 15, 737-746). For example, cereblon (CRBN) forms an E3 ubiquitin ligase complex with damaged DNA binding protein 1 (DDB1) and Cullin-4A (CUL4A) to ubiquitinate a number of other proteins followed by the degradation via proteasomes. (Yi-An Chen, et al., Scientific Reports 2015, 5, 1-13). Immunomodulatory drugs (IMiDs), including thalidomide, lenalidomide, and pomalidomide, function as monovalent promoters of PPIs by binding to the cereblon (CRBN) subunit of the CRL4A^{CRBN} E3 ligase complex and recruiting neosubstrate proteins. (Matyskiela, M. E. et al., Nat Chem Biol 2018, 14, 981-987.) As a consequence, the ability of thalidomide, and its derivatives, to recruit CRBN has been widely applied in proteolysis-targeting chimeras (PROTACs) related studies (Christopher T. et al. ACS Chem. Biol. 2019, 14, 342-347; Honorine L. et al, ACS Cent. Sci. 2016, 2, 927-934). PROTACs have great potential to eliminate protein targets that are “undruggable” by traditional inhibitors or are non-enzymatic proteins. (Chu T T. et al., Cell Chem Biol. 2016; 23:453-461. Qin C. et al., J Med Chem 2018; 61: 6685-6704. Winter G E. et al., Science 2015; 348:1376-1381.) In the recent years, PROTACs as useful modulators promote the selective degradation of a wide range of target proteins have been reported in antitumor studies. (Lu J. et al., Chem Biol. 2015; 22(6):755-763; Ottis P. et al., Chem Biol. 2017; 12(4):892-898; Crews C. M. et al., J Med Chem. 2018; 61(2):403-404; Neklesa T. K. et al., Pharmacol Ther 2017, 174:138-144; Cermakova K. et al., Molecules, 2018.23(8); An S. et al., EBioMedicine, 2018; Lebraud H. et al., Essays Biochem. 2017; 61(5): 517-527; Sun Y. H. et al., Cell Res. 2018; 28:779-81; Toure M. et al., Angew Chem Int Ed Engl. 2016; 55(6):1966-1973; Yonghui Sun et al., Leukemia, volume 33, pages 2105-2110(2019); Shaodong Liu et al., Medicinal Chemistry Research, volume 29, pages 802-808(2020); and has been disclosed or discussed in patent publications, e.g., US20160045607, US20170008904, US20180050021, US20180072711, WO2002020740, WO2014108452, WO2016146985, WO2016149668, WO2016197032, WO2016197114, WO2017011590, WO2017030814, WO2017079267, WO2017182418, WO2017197036, WO2017197046, WO2017197051, WO2017197056, WO2017201449, and WO2018071606.

Epidermal growth factor receptor (EGFR) that belongs to the ErbB family is a transmembrane receptor tyrosine kinase (RTK), which plays a fundamentally key role in cell proliferation, differentiation, and motility (Y. Yarden, et al., Nat. Rev. Mol. Cell Biol. 2001; 2:127-137). Homo- or heterodimerization of EGFR and other ErbB family members activates cytoplasmic tyrosine kinase domains to initiate intracellular signaling. Overexpression or activating mutations of EGFR are associated the development of many types of cancers, such as pancreatic cancer, breast cancer, glioblastoma multiforme, head and neck cancer, and non-small cell lung cancer (Yewale C., et al. Biomaterials. 2013, 34 (34): 8690-8707). The activating mutations in the EGFR tyrosine kinase domain (L858R mutation and exon-19 deletion) have been identified as oncogenic drivers for NSCLC (Konduri, K., et al. Cancer Discovery 2016, 6 (6), 601-611). The first-generation EGFR tyrosine kinase inhibitors (EGFR-TKIs) gefitinib and erlotinib have approved for NSCLC patients with EGFR activation mutations (M. Maemondo, N. Engl. J. Med. 362 (2010) 2380-2388). Although most patients with EGFR mutant NSCLC respond to these therapies, patients typically develop resistance after an average of one year on treatment. There are several mechanisms of acquired resistance to gefitinib and erlotinib, including a secondary threonine 790 to methionine 790 mutation (T790M), is also called “gatekeeper” T790M mutation (Xu Y., et al. Cancer Biol Ther. 2010, 9 (8): 572-582). Therefore, the second-generation EGFR-TKIs afatinib and the third-generation EGFR-TKIs osimertinib (AZD9291) were developed as irreversible EGFR inhibitors that bind to Cys797 for the treatment of patients with T790M mutation. In particular, osimertinib that largely spares WT EGFR has achieved greater clinical response rate in NSCLC patients with EGFR T790M. However, several recent studies have reported a tertiary Cys797 to Ser797 (C797S) point mutation with osimertinib clinical therapy (Thress K S, et al. Nat. Med. 2015, 21 (6): 560-562). There is a need for drugs which can overcome EGFR (C797S) resistance obstacle in non-small cell lung cancer (NSCLC). EGFR-Targeting PROTACs serve as a potential strategy to overcome drug resistance mediated by these mutants, which has been disclosed or discussed in patent publications, e.g. WO2018119441, WO2019149922, WO2019183523, WO2019121562 and US20190106417.

Although, a number of EGFR-targeting PROTACs which were designed to degrade EGFR mutant proteins have been published (Zhang X., et al. Eur. J. Med. Chem. 2020, 192, 112199; Zhang H, et al. Eur. 35 J. Med. Chem. 2020, 189, 112061; Lu X, Med. Res. Rev. 2018, 38(5):1550-1581. He K., et al. Bioorg. Med. Chem. Lett. 2020, 15, 127167). Most of the published molecules are based on first, second, and third generation of EGFR inhibitors. However, there were no data which showed those EGFR-Targeting PROTACs degrading all the main EGFR mutations, Such as Del19, L858R, Del19/T790M, L858R/T790M, Del19/T790M/C797S, L858R/T790M/C797S.

The present application provides novel bifunctional compounds and compositions for the treatment of serious diseases.

### SUMMARY OF THE INVENTION

One objective of the present invention is to provide compounds and derivatives formed by conjugating EGFR inhibitor moieties with E3 ligase ligand moieties, which function to recruit targeted proteins to E3 ubiquitin ligase for degradation, and methods of preparation and uses thereof.
Aspect 1. A compound of Formula (I):

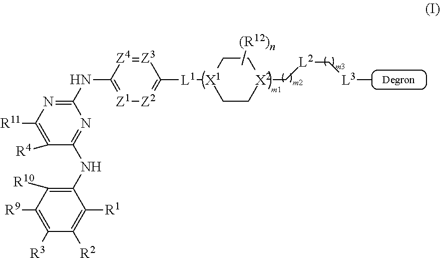

or a N-oxide thereof, or a pharmaceutically acceptable salt thereof, or a stereoisomer thereof, or a deuterated analog thereof, or a prodrug thereof

wherein:

R¹ is selected from —P(O)R^{1a}R^{1b}, —SO₂R^{1a}, —SO₂—NR^{1a}R^{1b} or —N(R^{1a})—SO₂R^{1b};

R^{1a} and R^{1b} are each independently selected from hydrogen, halogen, —C₁-C₈alkyl or C₃-C₈cycloalkyl, said —C₁-C₈alkyl or C₃-C₈cycloalkyl is optionally substituted with at least one halogen;

R² and R³ are each independently selected from hydrogen, halogen, —C₁-C₈alkyl, —C₃-C₈cycloalkyl, 3- to 8-membered heterocyclyl, C₆-C₁₂aryl, 5- to 12-membered heteroaryl, —CN, —OR^{2a}, —SO₂R^{2a}, —SO₂NR^{2a}R^{2b}, —C(O)R^{2a}, —CO₂R^{2a}, —C(O)NR^{2a}R^{2b}, —NR^{2a}R^{2b}, —NR^{2a}COR^{2b}, —NR^{2a}CO₂R^{2b}, or —NR^{2a}SO₂R^{2b}; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{2d}, or

R² and R³ together with the carbon atoms to which they are attached, form a 5 or 6-membered unsaturated or saturated ring, said ring comprising 0-3 heteroatoms independently selected from nitrogen, oxygen or sulfur; said ring is optionally substituted with at least one substituent R^{2e};

R^{2e}, at each occurrence, is independently hydrogen, halogen, —C₁-C₈alkyl, —C₁-C₈alkoxy, —C₃-C₈cycloalkyl, 3- to 8-membered heterocyclyl, C₆-C₁₂aryl, 5- to 12-membered heteroaryl, oxo (═O), —OR^{2a}, thioxo (═S), —SR^{2a}, —CN, —SO₂R^{2a}, —SO₂NR^{2a}R^{2b}, —C(O)R^{2a}, —CO₂R^{2a}, —C(O)NR^{2a}R^{2b}, —NR^{2a}R^{2b}, —NR^{2a}COR^{2b}, —NR^{2a}CO₂R^{2b} or —NR^{2a}SO₂R^{2b}; each of —C1-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent R^{2d};

R^{2a} and R^{2b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;

R^{2d}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;

R⁴ is selected from hydrogen, halogen, —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl, 5- to 12-membered heteroaryl, —CN, —SO₂R^{4a}, —SO₂NR^{4a}R^{4b}, —C(O)R^{4a}, —CO₂R^{4a}, —C(O)NR^{4a}R^{4b}, —NR^{4a}R^{4b}, —NR^{4a}COR^{4b}, —NR^{4a}CO₂R^{4b} or —NR^{4a}SO₂R^{4b}; each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{4c}, —SO₂R^{4c}, —SO₂NR^{4c}R^{4d}, —C(O)R^{4c}, —CO₂R^{4c}, —C(O)NR^{4c}R^{4d}, —NR^{4c}R^{4d}, —NR^{4c}COR^{4d}, —NR^{4c}CO₂R^{4d} or —NR^{4c}SO₂R^{4d};

R^{4a}, R^{4b}, R^{4c} and R^{4d} are each independently hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;

R⁹, R¹⁰, R¹¹ and R¹² are each independently selected from hydrogen, halogen, —C₁-C₈alkyl, —NR^{9a}R^{9b}, —OR^{9a}, —C₃-C₈cycloalkyl, 3- to 8-membered heterocyclyl, —C₆-C₁₂aryl, 5- to 12-membered heteroaryl, oxo (═O) or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{9c}; or

two R¹² together with the carbon atoms to which they are attached, form a 3- to 12-membered ring, said ring comprising 0-3 heteroatoms independently selected from nitrogen, oxygen or sulfur; said ring is optionally substituted with at least one substituent R^{9c};

R^{9a} and R^{9b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5 to 12-membered heteroaryl is optionally substituted with at least one substituent R^{9d}; or

R^{9c} and R^{9d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl;

Z¹, Z², Z³ and Z⁴ are each independently selected from —CR^{Z}, or N;

R^{Z}, at each occurrence, is independently selected from hydrogen, halogen, —C₁-C₈alkyl, —NR^{Za}R^{Zb}, —OR^{Za}, —SR^{Za}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{Zc};

R^{Za} and R^{Zb} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5 to 12-membered heteroaryl is optionally substituted with at least one substituent R^{Zd};

R^{Zc} and R^{Zd} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;

L¹ is selected from a single bond, —O—, —SO₂—, —C(O)—, —NR^{L1a}—, —C₃-C₈cycloalkylene-, *^{L1}—O—C₁-C₈alkylene-**^{L1}, *^{L1}—C₁-C₈alkylene-O—**^{L1}, *^{L1}—SO₂—C₁-C₈alkylene-**^{L1}, *^{L1}—C₁-C₈alkylene-SO₂—**^{L1}, *^{L1}—C(O)—C₁-C₈alkylene-**^{L1}, *^{L1}—C₁-C₈alkylene-C(O)—**^{L1}, *^{L1}—NR^{L1a}—C₁-C₈alkylene-**^{L1}, *^{L1}—C₁-C₈alkylene- NR^{L1a}—**^{L1}, *^{L1}—NR^{L1a}C(O)—**^{L1}, *^{L1}—C(O)NR^{L1a}—**^{L1}, —C₁-C₈alkylene-, —C₂-C₈alkenylene-, —C₂-C₈alkynylene-, —[O(CR^{L1a}R^{L1b})ₘ₄]m₅-,

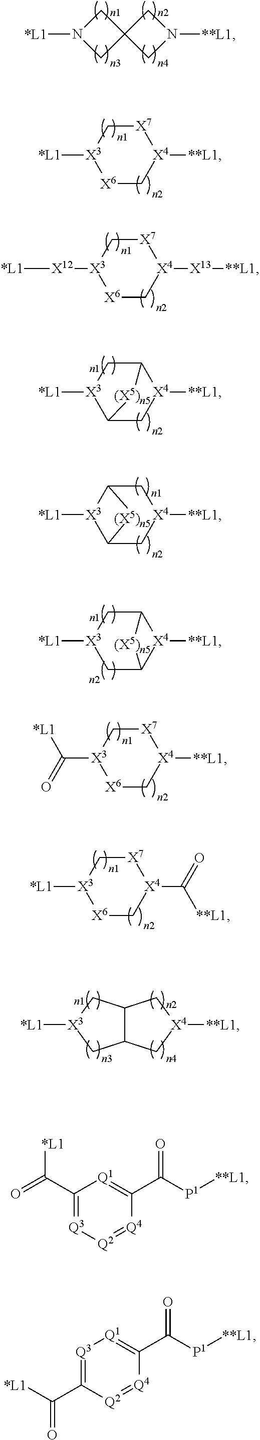

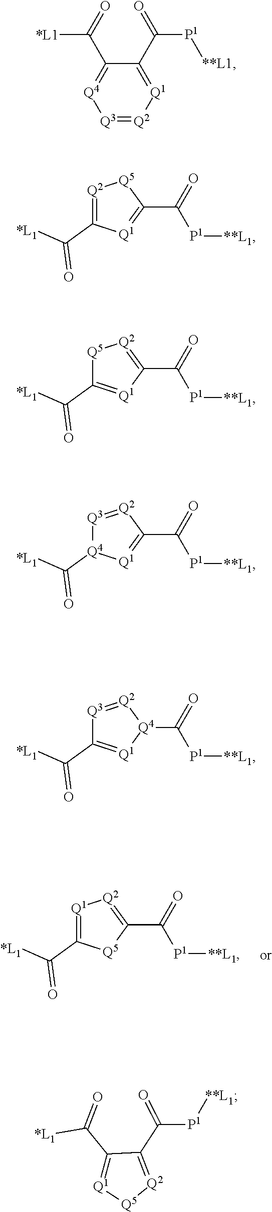

wherein each of said —C₃-C₈cycloalkylene-, *^{L1}-O—C₁-C₈alkylene-**^{L1}, *^{L1}—C₁-C₈alkylene-O—**^{L1}, *^{L1}-SO₂—C₁-C₈alkylene-**^{L1}, *^{L1}—C₁-C₈alkylene-SO₂—**^{L1}, *^{L1}—C(O)—C₁-C₈alkylene-**^{L1}, *^{L1}—C₁-C₈alkylene-C(O)—**^{L1}, *^{L1}—NR^{L1a}—C₁-C₈alkylene-**^{L1}, *^{L1}—C₁-C₈alkylene-NR^{L1a}—**^{L1}, —C₁-C₈alkylene-, —C₂-C₈alkenylene-, —C₂-C₈alkynylene-,

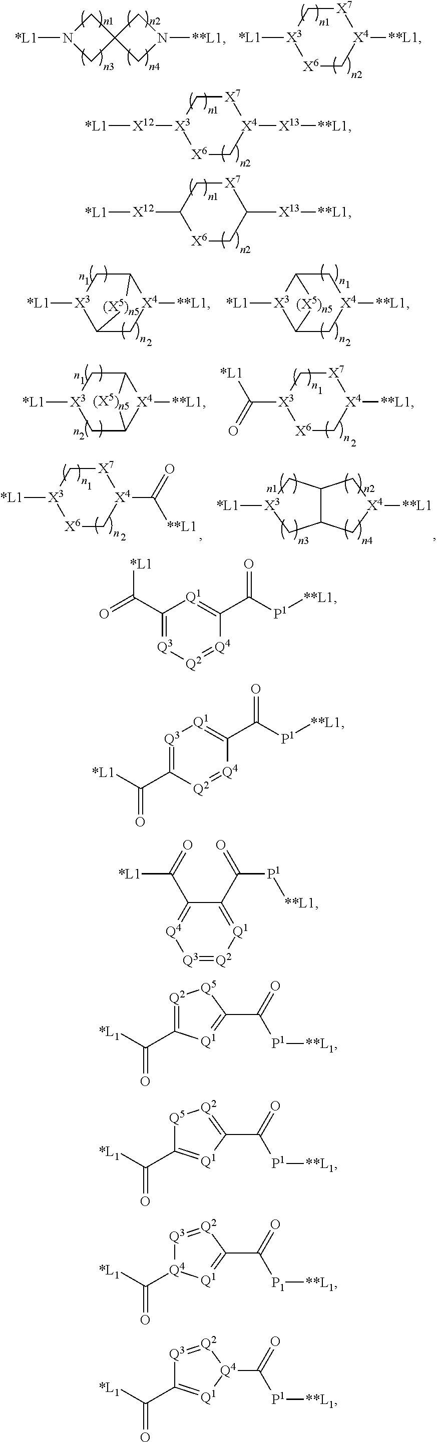

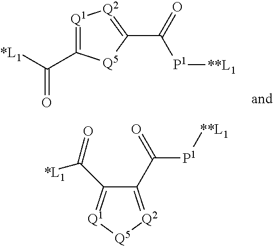

is optionally substituted with at least one R^{L1c};

wherein *^{L1} refers to the position attached to the

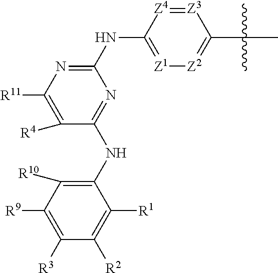

moiety, and **^{L1} refers to the position attached to the

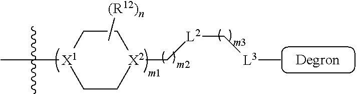

moiety;

R^{L1a} and R^{L1b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L1d};

each of said R^{L1c} and R^{L1d} are independently oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or

two R^{L1c} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;

L² is selected from a single bond, —O—, —SO₂—, —C(O)—, —NR^{L2a}—, —C₃-C₈cycloalkylene-, *^{L2}-O—C₁-C₈alkylene-**^{L2}, *^{L2}—C₁-C₈alkylene-O—**^{L2}, *^{L2}—SO₂—C₁-C₈alkylene-**^{L2}, *^{L2}—C₁-C₈alkylene-SO₂—**^{L2}, *L C(O)—C₁-C₈alkylene-**^{L2}, *^{L2}—C₁-C₈alkylene-C(O)—**^{L2}, *^{L2}-NR^{L2a}—C₁-C₈alkylene-**^{L2}, *^{L2}—C₁-C₈alkylene-NR^{L2a}—**^{L2}, *^{L2}-NR^{L2a}C(O)—**^{L2}, *^{L2}—C(O)NR^{L2a}—**^{L2} C₁-C₈alkylene-, —C₂-C₈alkenylene-, —C₂-C₈alkynylene-, —[O(CR^{L2a}R^{L2b})ₘ₄]ₘ₅—,

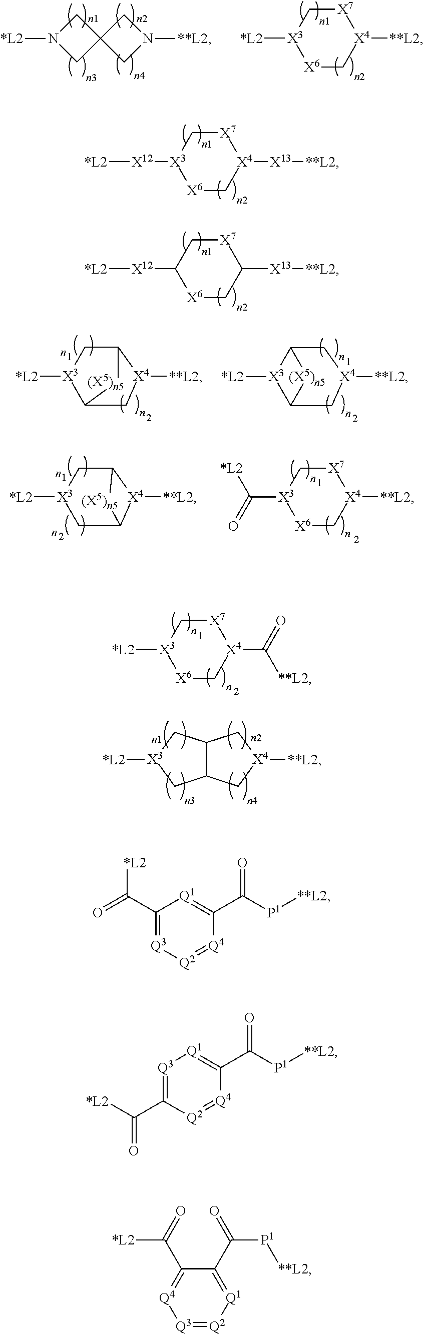

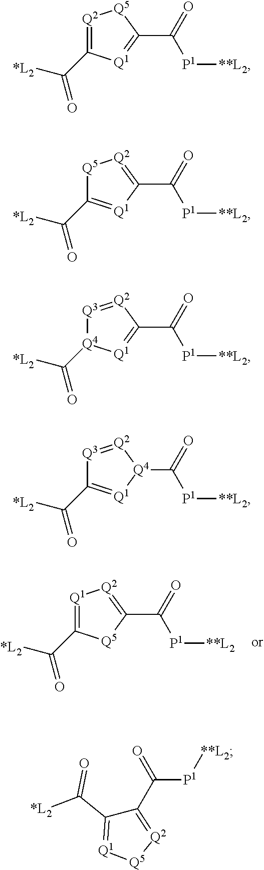

wherein each of said —C₃-C₈cycloalkylene-, *^{L2}-O—C₁-C₈alkylene-**^{L2}, *^{L2}—C₁-C₈alkylene-O—**^{L2}, *^{L2}—SO₂C₁-C₈alkylene-**^{L2}, *^{L2}—C₁-C₈alkylene-SO₂r**^{L2}, *^{L2}—C(O)—C₁-C₈alkylene-**^{L2}, *^{L2}—C₁-C₈alkylene- C(O)—**^{L2}, *^{L2}—NR^{L2a}C1-C₈alkylene-**^{L2}, *^{L2}—C₁-C₈alkylene-NR^{L2a}—**^{L2}, —C₁-C₈alkylene-, —C₂-C₈alkenylene-, —C₂-C₈alkynylene-,

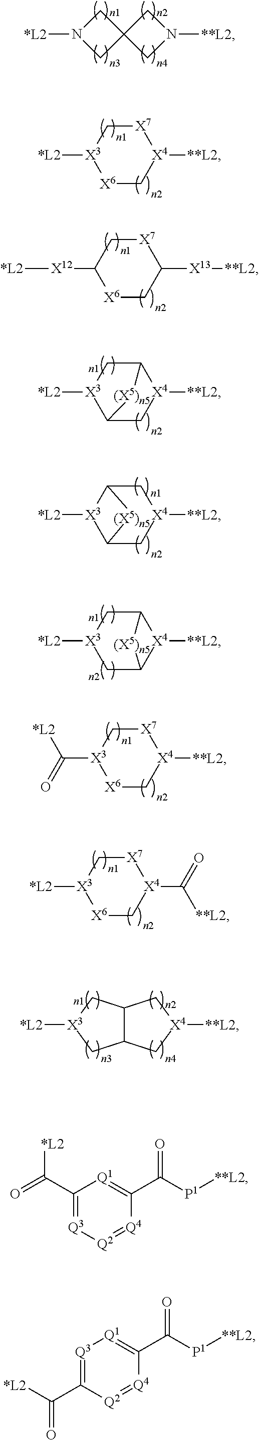

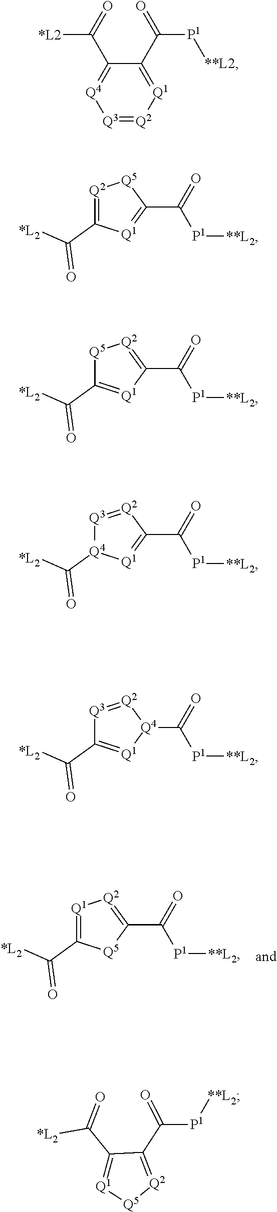

is optionally substituted with at least one substituent R^{L2c};

wherein *^{L2} refers to the position attached to

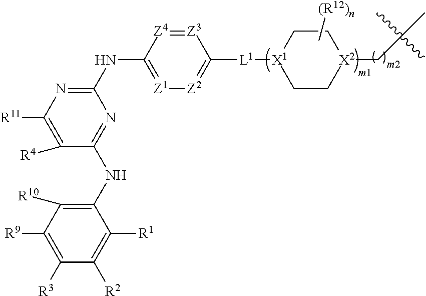

moiety, and **^{L2} refers to the position attached to the

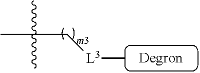

R^{L2a} and R^{L2b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L2d};

each of said R^{L2c} and R^{L2d} is independently selected from oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or

two R^{L2c} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;

L³ is selected from a single bond, —O—, —SO₂—, —C(O)—, —NR^{L3a}—, —C₃-C₈cycloalkylene-, *^{L3}—O—C₁-C₈alkylene-**^{L3}, *^{L3}—C₁-C₈alkylene-O—**^{L3}, *^{L3}—SO₂—C₁-C₈alkylene**^{L3}, *^{L3}—C₁-C₈alkylene-SO₂—**^{L3}, *^{L3} —C(O)—C₁-C₈alkylene-**^{L3}, *^{L3}—C₁-C₈alkylene-C(O)—**^{L3}, *^{L3}—NR^{L3a}—C₁-C₈alkylene_**^{L3}, *^{L3}—C₁-C₈alkylene- NR^{L3a}—**^{L3}, *^{L3}—NR^{L3a}C(O)—**^{L3}, *^{L3}—C(O)NR^{L3a}—**^{L3}, —C₁-C₈alkylene-, —C₂-C₈alkenylene-, —C₂-C₈alkynylene-, —[O(CR^{L3a}R^{L3b})ₘ₄]ₘ₅—

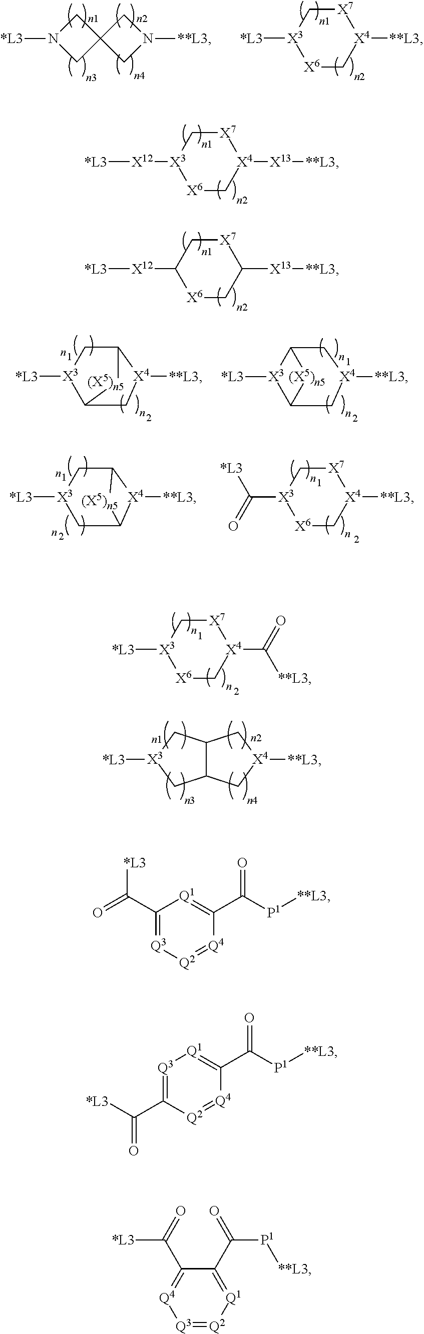

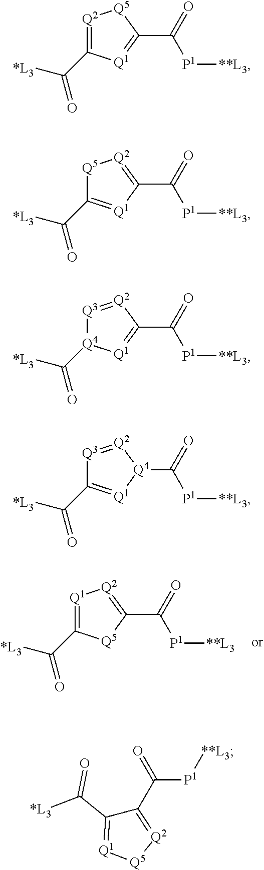

wherein each of said —C₃-C₈cycloalkylene-, *^{L3}—O—C₁-C₈alkylene-**^{L3}, *^{L3}—C₁-C₈alkylene-O—**^{L3}, *^{L3}—SO₂—C₁-C₈alkylene-**^{L3}, *^{L3}—C₁-C₈alkylene-SO₂**^{L3}, *^{L3}—C(O)—C₁-C₈alkylene**^{L3},*^{L3}—C₁-C₈alkylene- C(O)—**^{L3}, *^{L3}—NR^{L3a}—C₁-C₈alkylene-**^{L3}, *_{L3}—C₁-C₈alkylene-NR^{L3a_}**^{L3}, —C₁-C₈alkylene-, —C₂-C₈alkenylene-, —C₂-C₈alkynylene-,

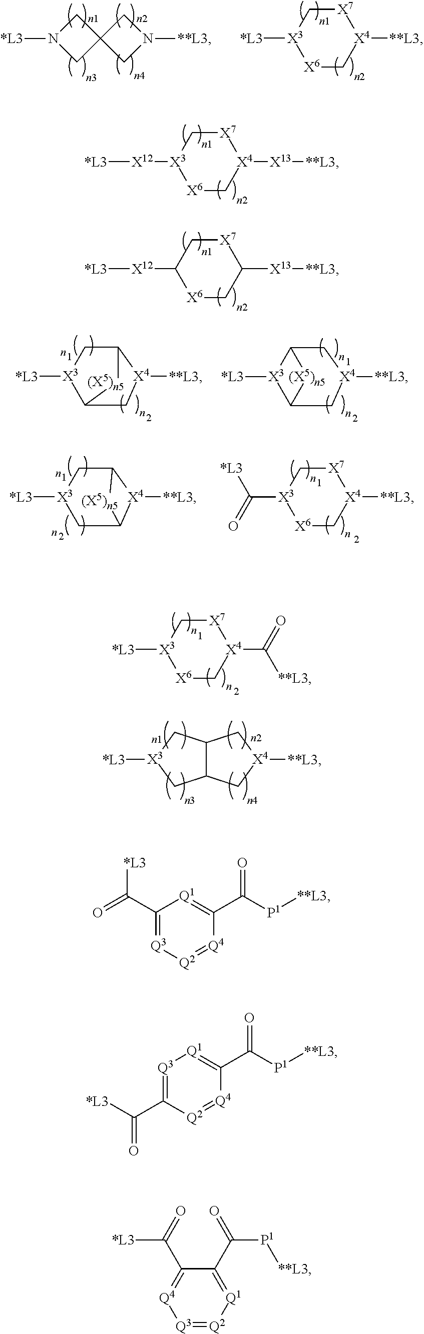

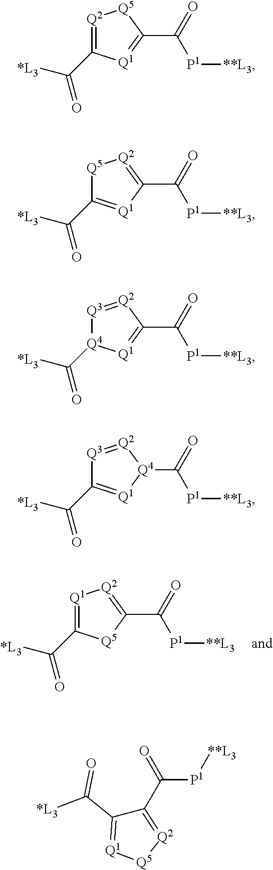

is optionally substituted with at least one substituent R^{L3c};

wherein *^{L3} refers to the position attached to

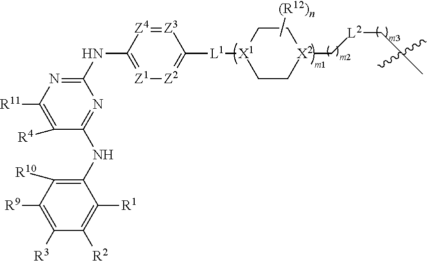

moiety, and **^{L3} refers to the position attached to the

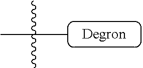

R^{L3a} and R^{L3b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L3d};

each of said R^{L3c} and R^{L3d} is independently selected from oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or

two R^{L3c} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl;

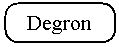

is selected from

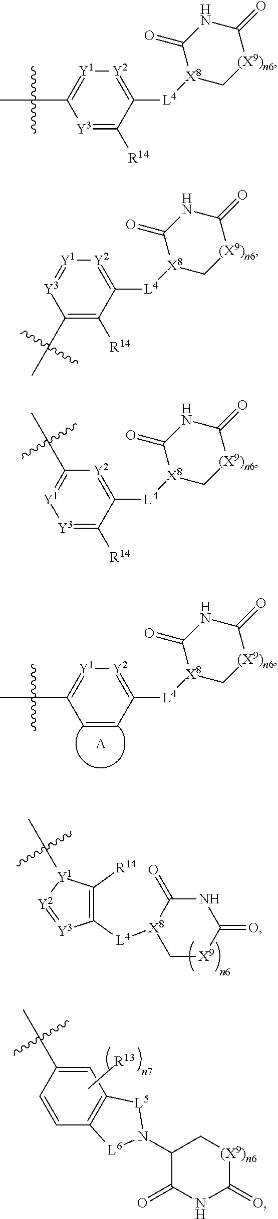

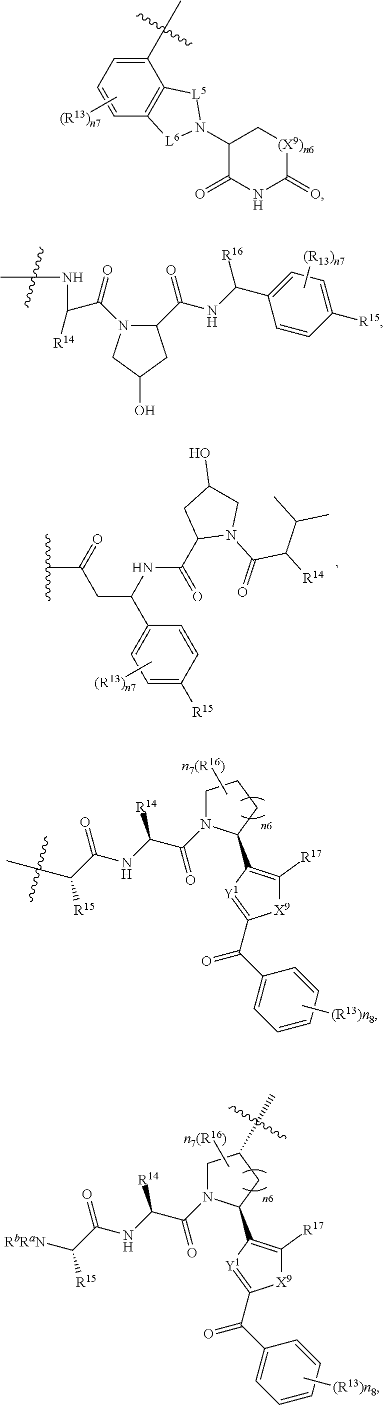

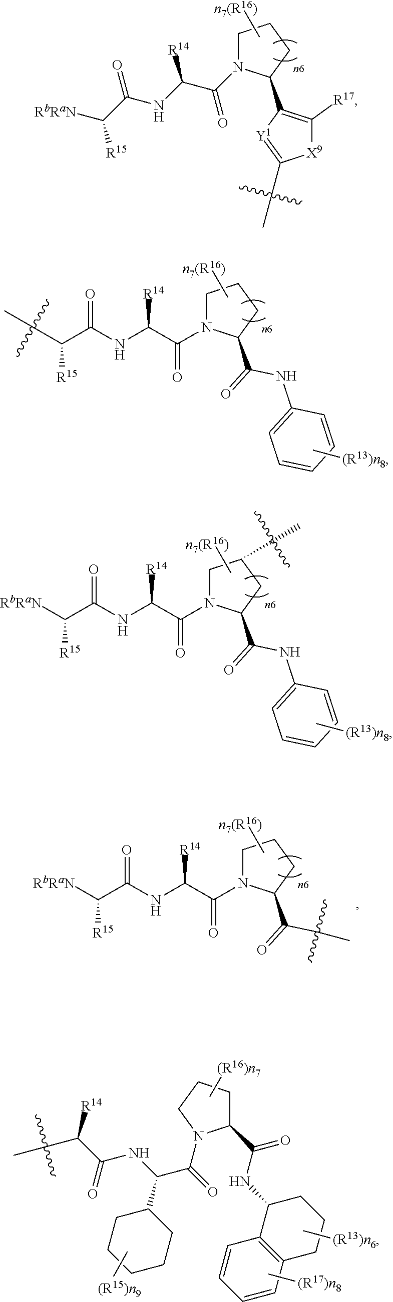

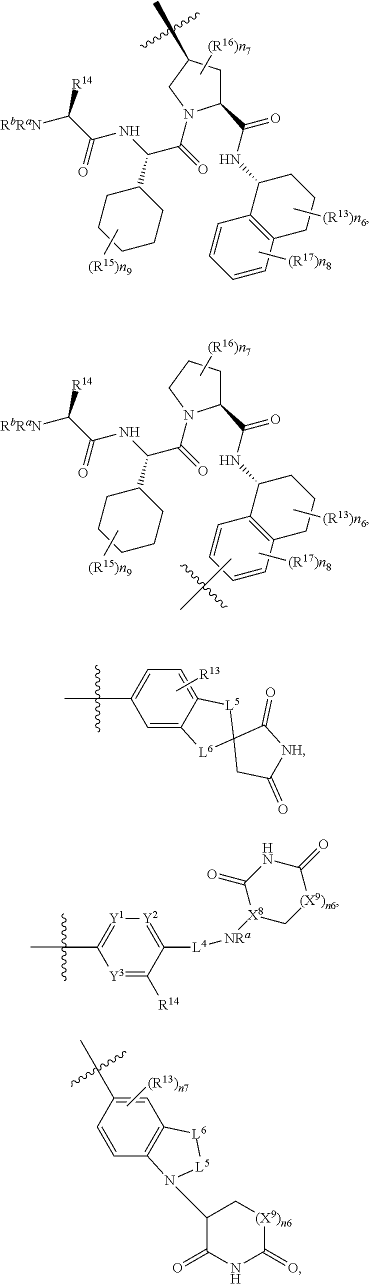

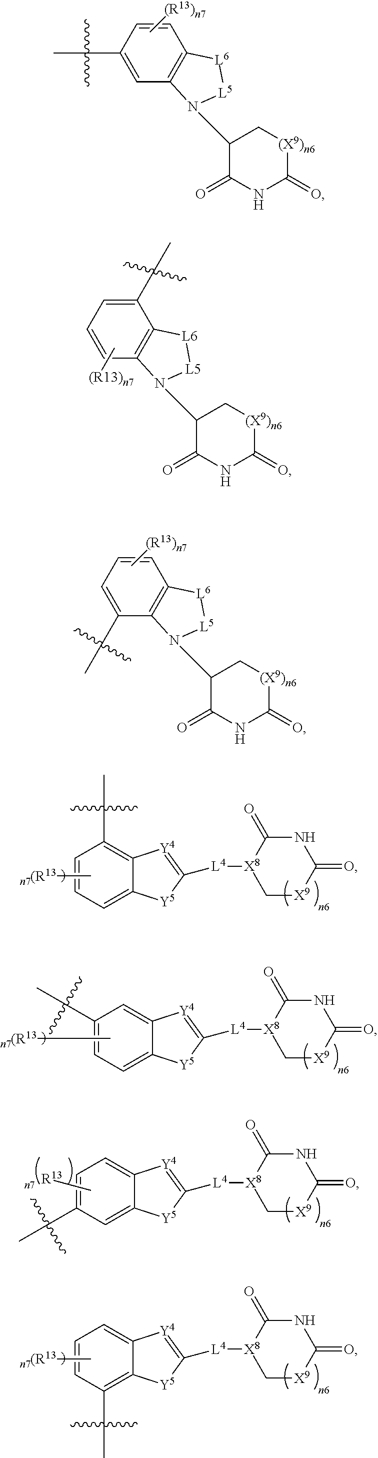

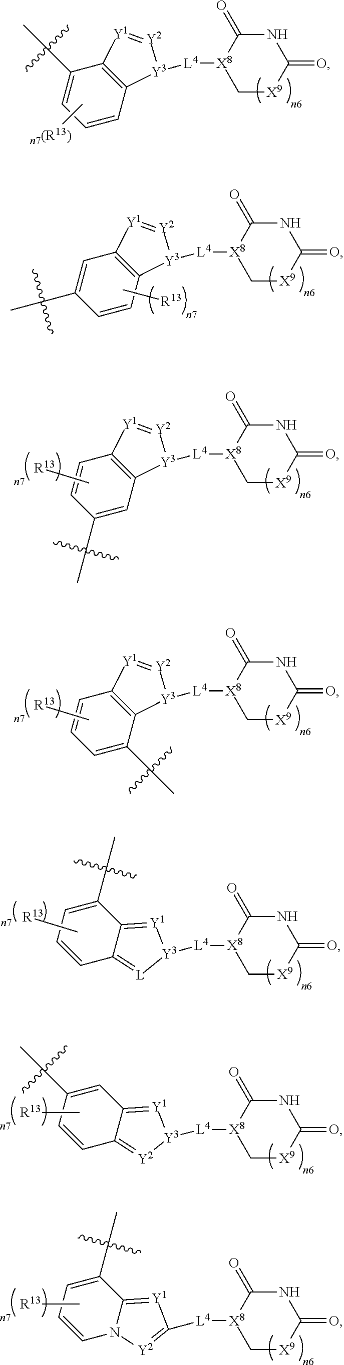

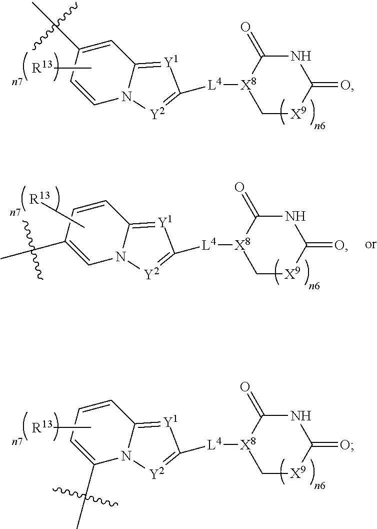

Ring A is selected from 3- to 12-membered cycloalkyl, 3- to 12-membered heterocyclyl, aryl, or heteroaryl;

R¹³, R¹⁴, R¹⁵, R¹⁶ and R¹⁷are each independently selected from hydrogen, halogen, CN, —C₁-C₈alkyl, —C₁-C₈alkoxy, C₃-C₈cycloalkyl, 3- to 8-membered heterocyclyl, C₆-C₁₂aryl or 5- to 12-membered heteroaryl; said each —C₁-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent halogen, —C₁-C₈alkyl, C₁-C₈alkoxy-C₁-C₈alkyl-, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;

X¹, X², X³, X⁴ and X⁸ are each independently selected from —CR^{a}, or N;

X⁵, X⁶, X⁷ and X⁹ are each independently selected from —NR^{a}—, —O—, —S— and —CR^{a}R^{b}—.

X¹² and X¹³ are each independently selected from —C(O)—. —NR^{a}— and —O—;

L⁴, L⁵ and L⁶ are each independently selected from a single bond, —O—, —NR^{a}—, —(CR^{a}R^{b})n₈-, —O(CR^{a}R^{b})n₈-, —NR^{a}(CR^{a}R^{b})n₈- or —C(O)—;

Q¹, Q², Q³, Q⁴, Y¹, Y², Y³ and Y⁴ are each independently selected from CR^{a} or N;

Y⁵ is selected from NR^{a}, O or S;

Q⁵ is each independently selected from —O—, —NR^{a}—, —CR^{a}R^{b}—, —S— or —C(O)—;

P¹ is a single bond, —O—, —NR^{a}—, —CR^{a}R^{b}—, —S—, —SO— or —SO₂—;

at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or

R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;

m₁ is 0, 1 or 2;

m₂ and m₃ are each independently 0, 1, 2, 3, 4, 5, 6, 7 or 8;

m₄ and m₅ are each independently 0, 1, 2 or 3;

n, n₁, n₂, n₃, n₄ and n₅ are each independently 0, 1, 2 or 3; and

n₆, n₇, n₈ and n₉ are each independently 0, 1, 2, 3 or 4.

Aspect 2. The compound of Aspect 1, wherein the compound is selected from formula (II), (III), (IV), (V), (VI) or (VII),

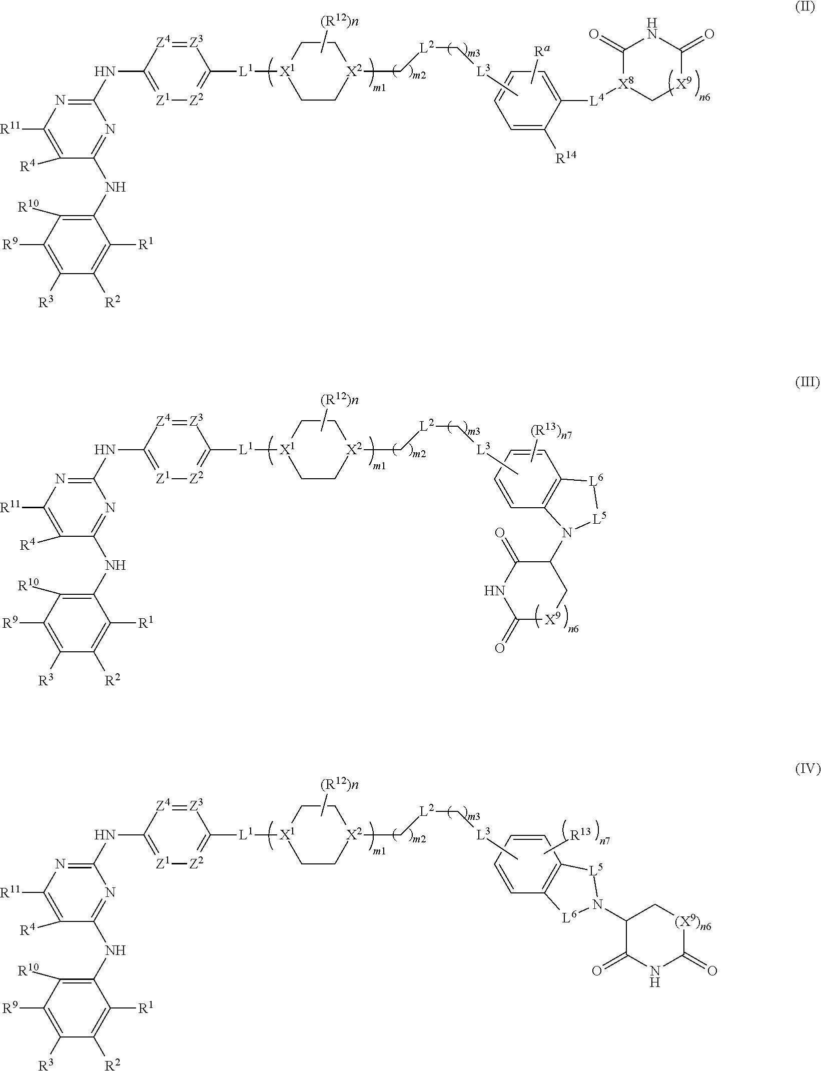

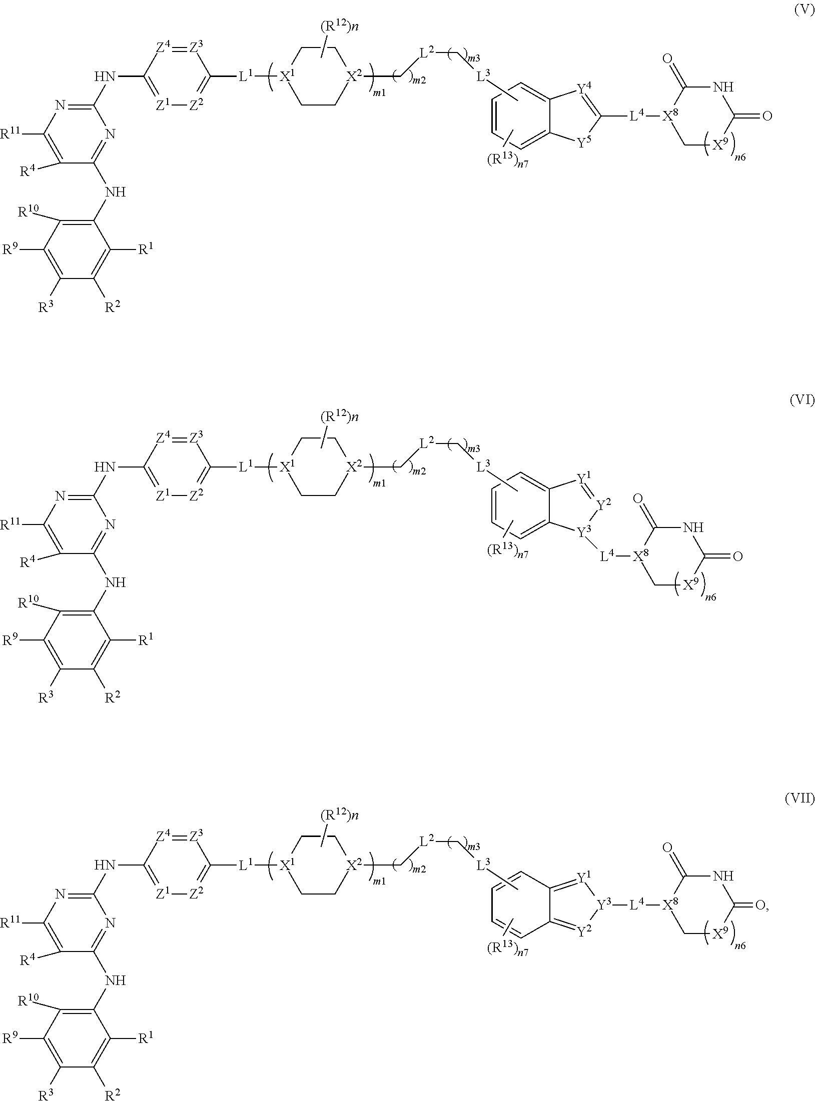

R¹, R², R³, R⁴, R⁹, R¹⁰, R¹¹, R¹², R¹³, R¹⁴, R^{a}, Z¹, Z², Z³, Z⁴, L¹, L², L³, L⁴, L⁵, L⁶, X¹, X², X⁸, X⁹, Y¹, Y², Y³, Y⁴, Y⁵, n, n6, n7, m1, m2 and m3 are each independently defined as Aspect 1.

Aspect 3. The compound of Aspects 1-2, wherein R¹ is selected from —P(O)R^{1a}R^{1b} or —N(R^{1a})—SO₂R^{1b}, wherein R^{1a} and R^{1b} are each independently selected from hydrogen, halogen, —C₁-C₈alkyl (preferably —CH₃, —C₂H₅, —C₃H₇, —C₄H₉ or —C₅H₁₁; more preferably —CH₃, —CH₂CH₃, —CH₂CH₂CH₃,-iso-C₃H₇, —CH₂CH₂CH₂CH₃,-iso-C₄H₉, -sec-C₄H₉ or -tert-C₄H₉) or C₃-C₈cycloalkyl (preferably cyclopropyl, cyclobutyl or cyclopentyl).

Aspect 4. The compound of any one of Aspects 1-3, wherein R₁ is selected from —P(O)(CH₃)₂, —NH—SO₂CH₃ or —N(CH₃)—SO₂CH₃.

Aspect 5. The compound of any one of Aspects 1-4, wherein R₁ is —P(O)(CH₃)₂.

Aspect 6. The compound of any one of Aspects 1-5, wherein R² and R³ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl, 5 to 12-membered heteroaryl, —CN, —OR^{2a}, —SO₂R^{2a}, —SO₂NR^{2a}R^{2b}, —C(O)R^{2a}, —CO₂R^{2a}, —C(O)NR^{2a}R^{2b}, —NR^{2a}R^{2b}, —NR^{2a}COR^{2b}, —NR^{2a}CO₂R^{2b}, or —NR^{2a}SO₂R^{2b}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl is optionally substituted with at least one substituent R^{2d}, or

R² and R³ together with the carbon atoms to which they are attached, form a 5 or 6-membered unsaturated or saturated ring, said ring comprising 0, 1, 2 or 3 heteroatoms independently selected from nitrogen, oxygen or sulfur; said ring is optionally substituted with at least one substituent R^{2e};

R^{2e}, at each occurrence, is independently —H, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl, oxo (═O), —CN, CF₃, CHF₂, CH₂F, thioxo (═S), —SCF₃, —SCHF₂, —SCH₂F, —SCH₂CF₃, —SCF₂CH₃, —SCF₂CF₃, —SO₂R^{2a}, —SO₂NR^{2a}R^{2b}, —C(O)R^{2a}, —CO₂R^{2a}, —C(O)NR^{2a}R^{2b}, —NR^{2a}R^{2b}, —NR^{2a}COR^{2b}, —NR^{2a}CO₂R^{2b} or —NR^{2a}SO₂R^{2b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl is optionally substituted with at least one substituent R^{2a}.

R^{2a} and R^{2b} are each independently selected from hydrogen, methyl, ethyl, propyl, butyl, pentyl, hexyl, heptyl, octyl, methoxy, ethoxy, propoxy, butoxy, pentoxy, hexoxy, heptyloxy, octyloxy, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;

R^{2d}, at each occurrence, is independently selected from halogen, —OH, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 7. The compound of any one of Aspects 1-6, wherein R² and R³ together with the carbon atoms to which they are attached, form a 5 or 6-membered unsaturated (preferred aromatic) or saturated ring, said ring comprising 1 or 2 nitrogen heteroatoms; said ring is optionally substituted with at least one substituent —H, —F, —Cl, —Br, —I, methyl, ethyl, propyl (n- or iso-), butyl (n-, iso- or t-), pentyl, methoxy, ethoxy, propoxy, butoxy, pentoxy, cyclopropyl, cyclobutyl, cyclopentyl, cyclohexyl, cycloheptyl, cyclooctyl, —CH₂OH, —SCH₃, —SC₂H₅, oxo (═O), thioxo (═S), —CF₃, —CHF₂, —CH₂F, —SCF₃, —OMe, —OC₂H₅, —CN, —C(O)CH₃,

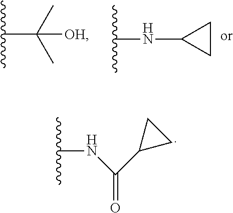

Aspect 8. The compound of any one of Aspects 1-7, wherein R² and R³ together with the carbon atoms to which they are attached, form a 6-membered unsaturated (preferred aromatic) ring, said ring comprising 1 or 2 nitrogen heteroatoms; said ring is optionally substituted with one substituent —H, —F, —Cl, —Br, —I, methyl, ethyl or cyclopropyl.

Aspect 9. The compound of any one of Aspects 1-8, wherein R⁴ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl, —C₂-C₈alkynyl or —C₁-C₈alkoxy; each of methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl or —C₂-C₈alkynyl is optionally substituted with —F, —Cl, —Br, —I, oxo (═O), or —CN.

Aspect 10. The compound of any one of Aspects 1-9, wherein R⁴ is hydrogen, —F, —Cl, —Br, —I, —CH₃, —CF₃, —CH₂F, —CHF₂, —C(O)OMe, —C(O)OEt, —C(O)OⁱPr or —C(O)O^{t}Bu.

Aspect 11. The compound of any one of Aspects 1-10, wherein R⁴ is hydrogen, —F, —Cl, —Br or —I.

Aspect 12. The compound of any one of Aspects 1-11, wherein R⁹, R¹⁰, R¹¹ and R¹² are each independently selected from hydrogen, —F, —Cl, —Br, —I, methyl, ethyl, propyl, butyl, pentyl, hexyl, heptyl, octyl, —NR^{9a}R^{9b}, —OR^{9a}, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), or —CN; each of -methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9c};

R^{9a} and R^{9b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9d};

R^{9c} and R^{9d} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 13. The compound of any one of Aspects 1-12, wherein R⁹, R¹⁰, R¹¹ and R¹² are each independently selected from hydrogen, —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, cyclohexyl, —CH₂OH, —CH₂OMe, oxo (═O), or —CN.

Aspect 14. The compound of any one of Aspects 1-13, wherein R⁹, R¹⁰, R¹¹ and R¹² are each independently selected from hydrogen, —CH₃, —F, —Cl, —Br or —I.

Aspect 15. The compound of any one of Aspects 1-11, wherein two R¹² together with the carbon atoms to which they are attached, form a 3, 4, 5, 6, 7 or 8-membered ring, said ring comprising 0, 1, 2 or 3 heteroatoms independently selected from nitrogen, oxygen or sulfur; said ring is optionally substituted with at least one substituent R^{9c};

R^{9c} is independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 16. The compound of any one of Aspects 1-11, wherein two R¹² together with the carbon atoms to which they are attached, form a 3, 4, 5, 6, 7 or 8-membered ring, preferably form a 3, 4, 5 or 6-membered ring, said ring comprising 0, 1, 2 or 3 heteroatoms independently selected from nitrogen, oxygen or sulfur; said ring is optionally substituted with at least one substituent —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, or cyclohexyl.

Aspect 17. The compound of any one of Aspects 1-5, wherein the

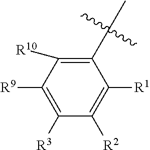

moiety is

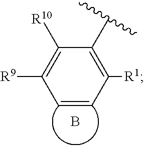

Ring B is a 5 or 6-membered unsaturated or saturated ring, said ring comprising 0, 1, 2 or 3 heteroatoms; said heteroatoms are independently selected from N, NR^{2e}, 0 or

said ring is optionally substituted with at least one substituent R^{2e}.

Aspect 18. The compound of any one of Aspects 1-17, wherein the

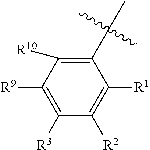

moiety is selected from

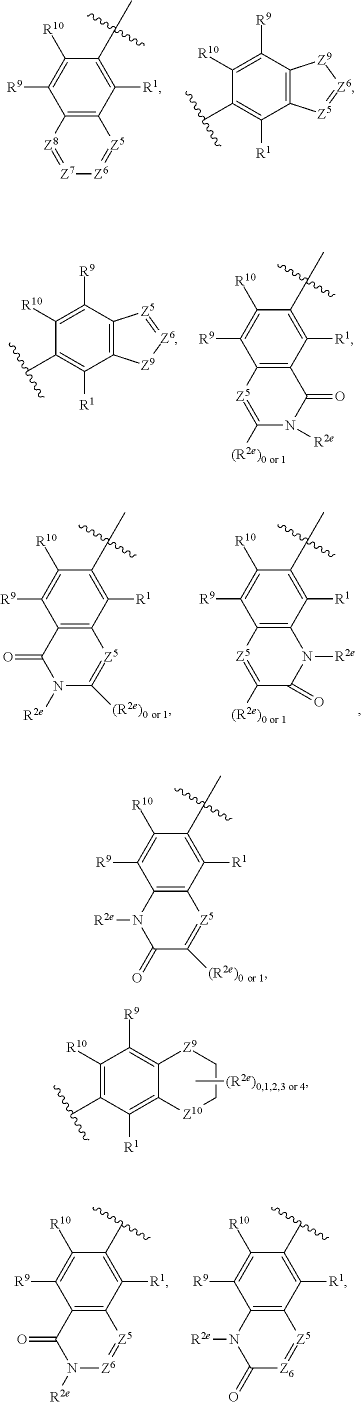

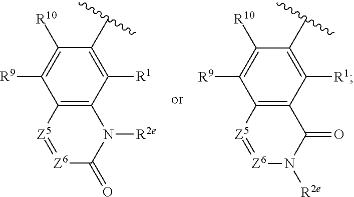

Z⁵, Z⁶, Z⁷ and Z⁸ are each independently selected from N, CH or CR^{2e};

Z⁹ and Z¹⁰ are each independently selected from O, S, NH or NR^{2e}.

Aspect 19. The compound of any one of Aspects 1-18, wherein the

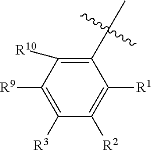

moiety is selected from

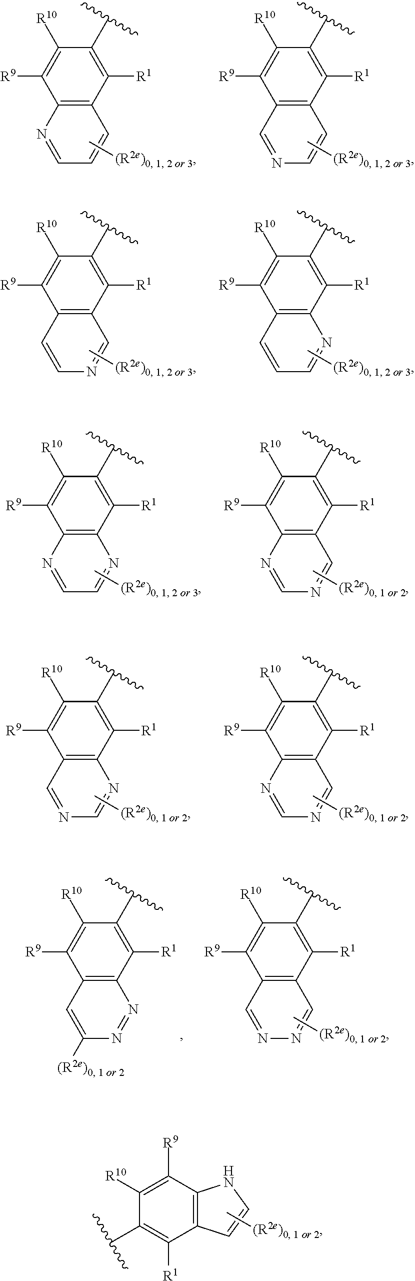

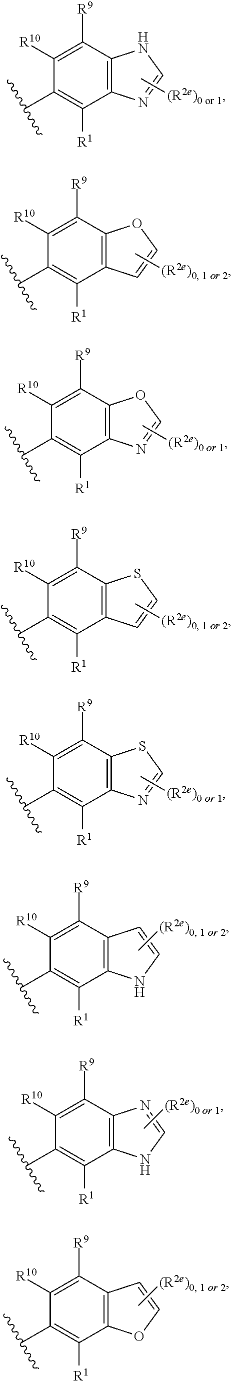

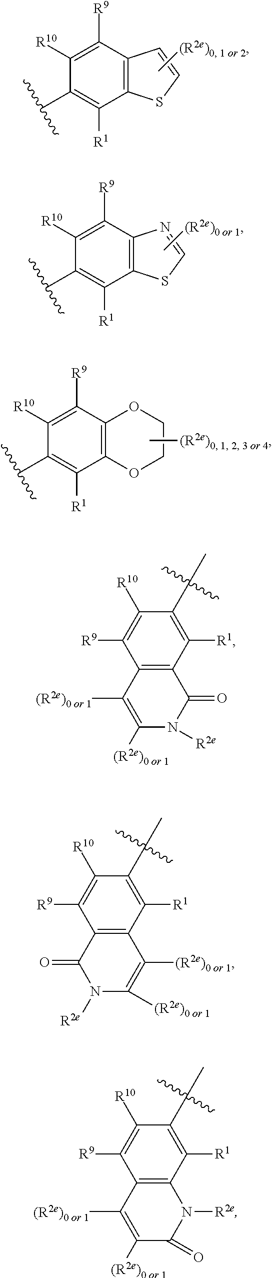

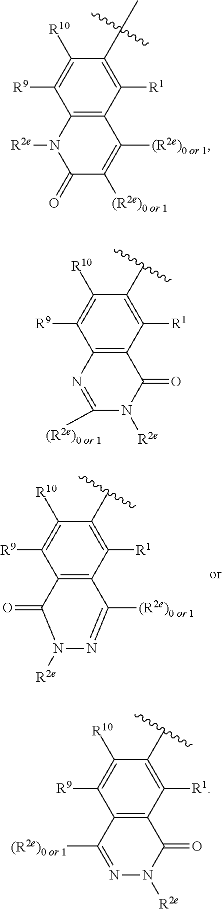

Aspect 20. The compound of any one of Aspects 1-19, wherein the

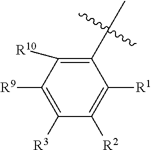

moiety is selected from

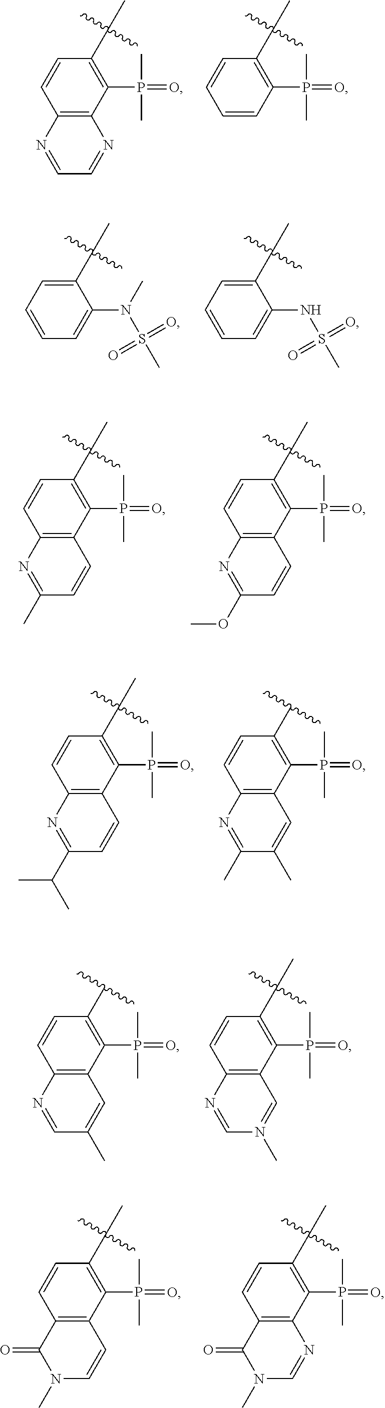

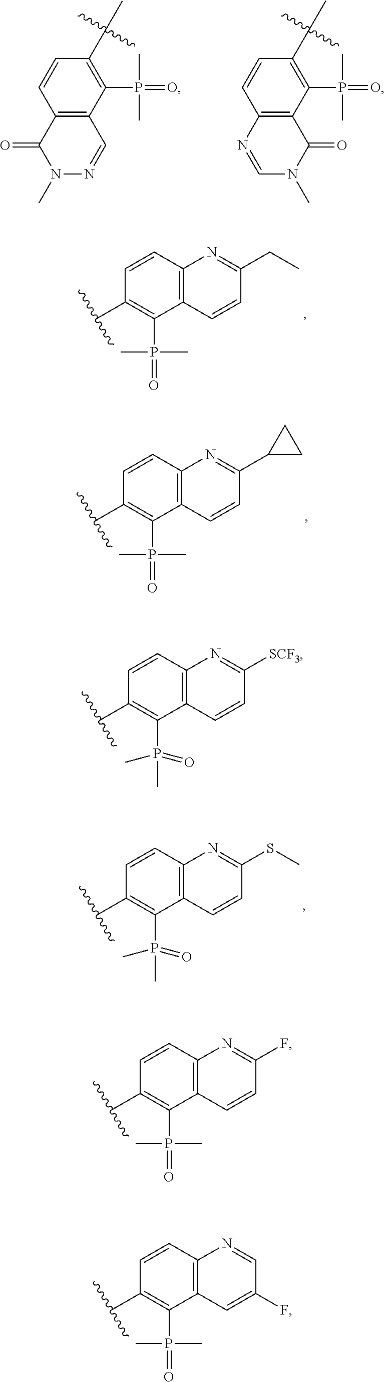

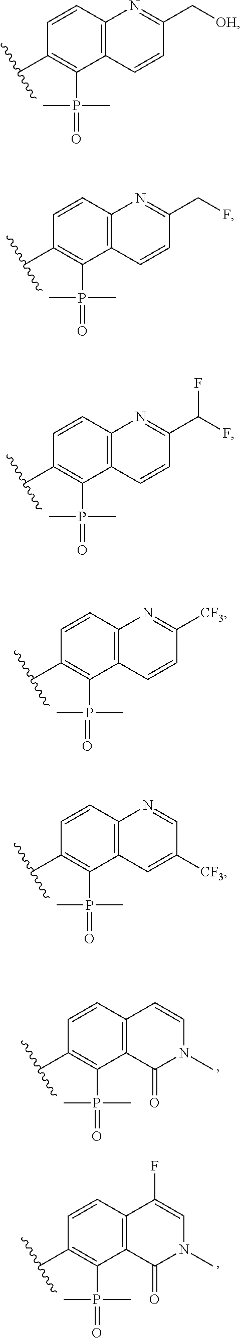

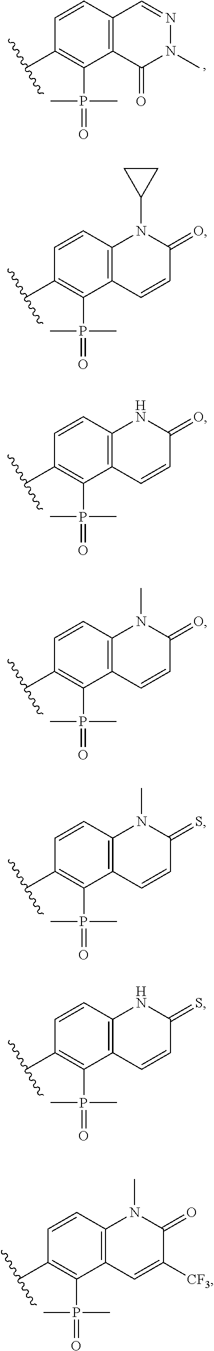

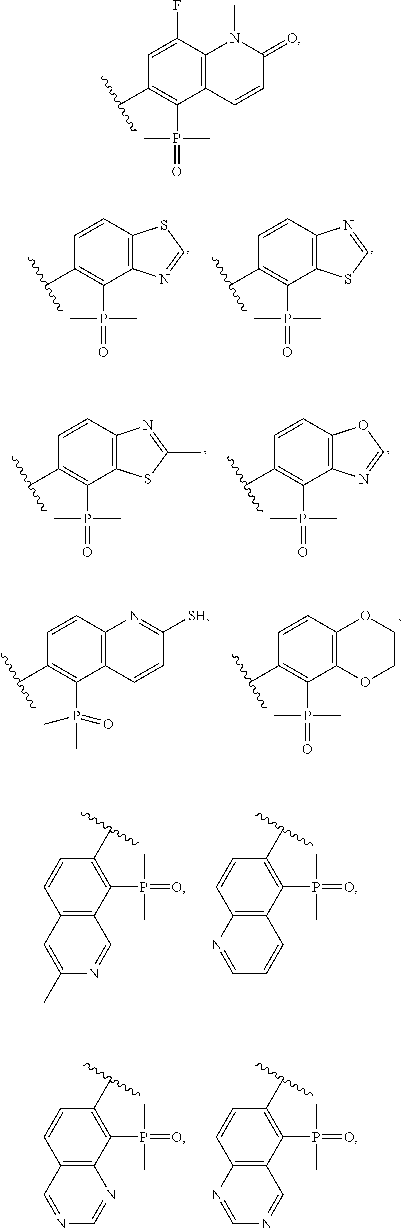

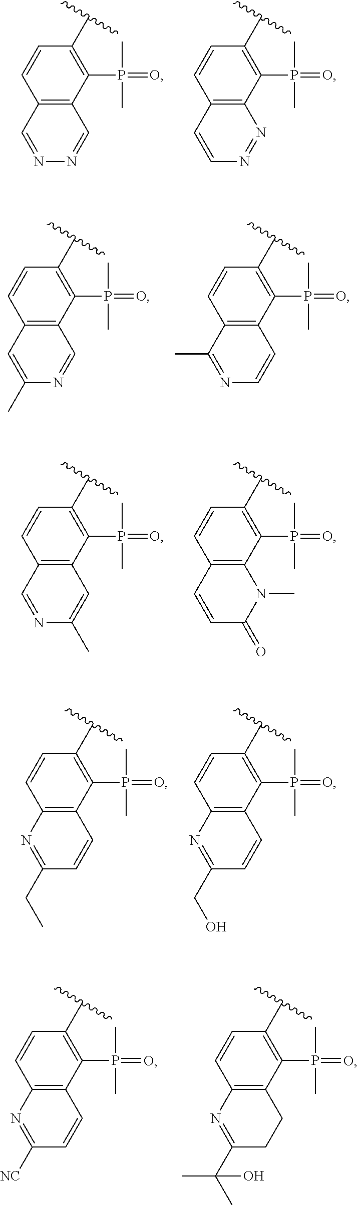

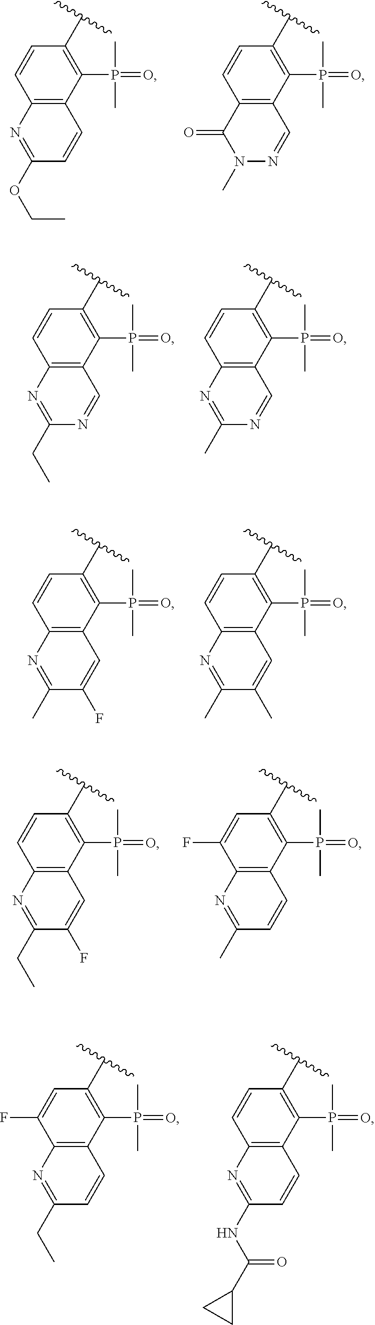

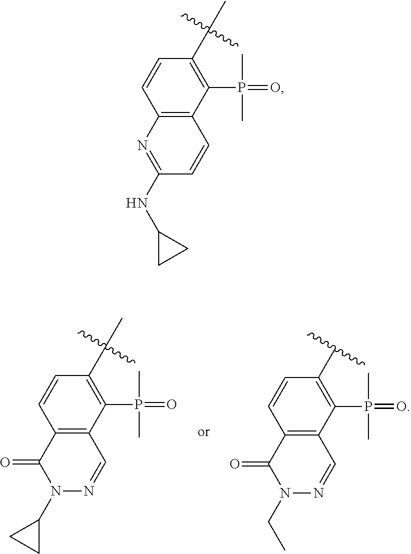 [SMILES: *c1ccc2nc(NC3CC3)ccc2c1P(C)(C)=O ; *c1ccc2c(=O)n(C3CC3)ncc2c1P(C)(C)=O ; "or" ; *c1ccc2c(=O)n(CC)ncc2c1P(C)(C)=O]

Aspect 21. The compound of any one of Aspects 1-20, wherein L¹ is selected from a single bond, —C1-C8alkylene-(preferably —CH₂—, —C₂H₄—, —C₃H₆—), —C(O)—C₁-C₈alkylene-(preferably —C(O)—CH₂—, —C(O)—C₂H₄—, —C(O)—C₃H₆—), —C₁-C₈alkylene-C(O)-(preferably —CH₂—C(O)—, —C₂H₄—C(O)—, —C₃H₆—C(O)—), —C(O)—, —O—, —N(CH₃)—, —NH—,

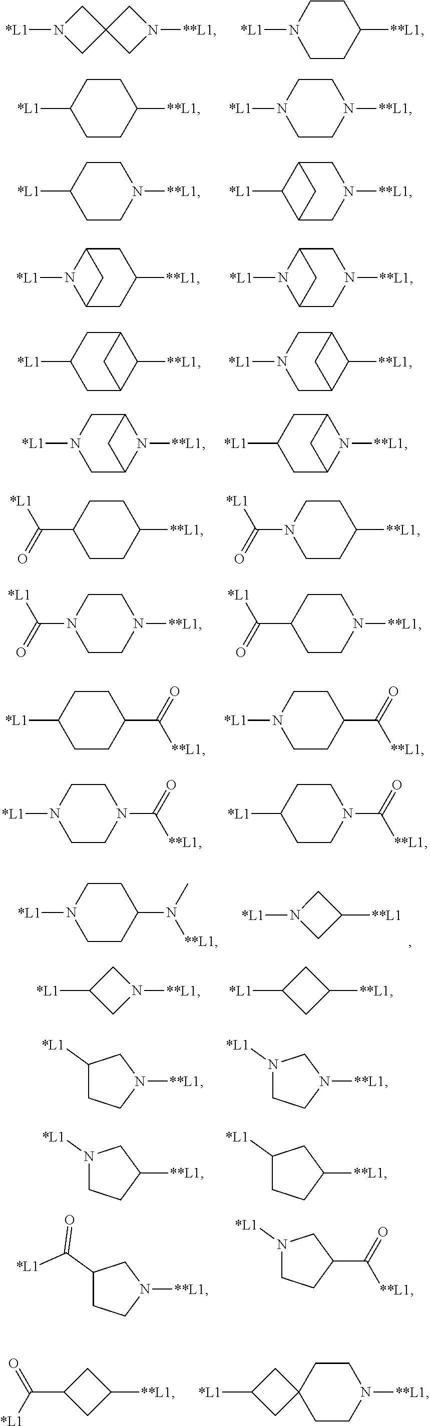

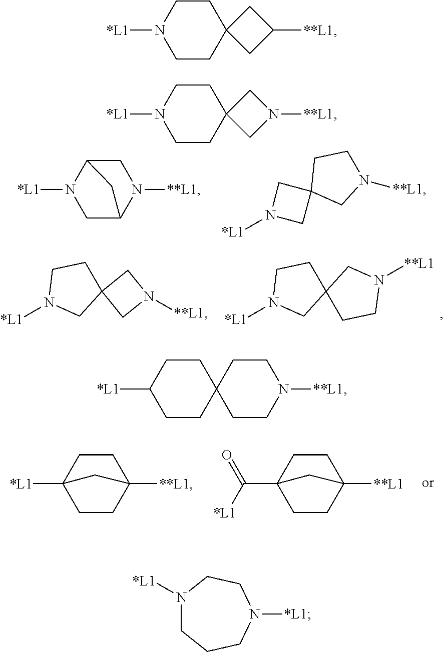

wherein each of said C₁-C₈alkylene-(preferably —CH₂—, —C₂H₄—, —C₃H₆—), *^{L1}—C(O)C₁-C₈alkylene_**^{L1} (preferably *^{L1}—C(O)—CH₂—**^{L1}, *^{L1}—C(O)—C₂H₄—**^{L1}, *^{L1}—C(O)—C₃H₆—**^{L1}), *^{L1}—C₁-C₈alkylene-C(O)—**^{L1} (preferably *^{L1}—CH₂—C(O)—**^{L1}, *^{L1}—C₂H₄—C(O)—**^{L1}, *^{L1}—C₃H₆—C(O)—**^{L}), —N(CH₃)—, —NH—,

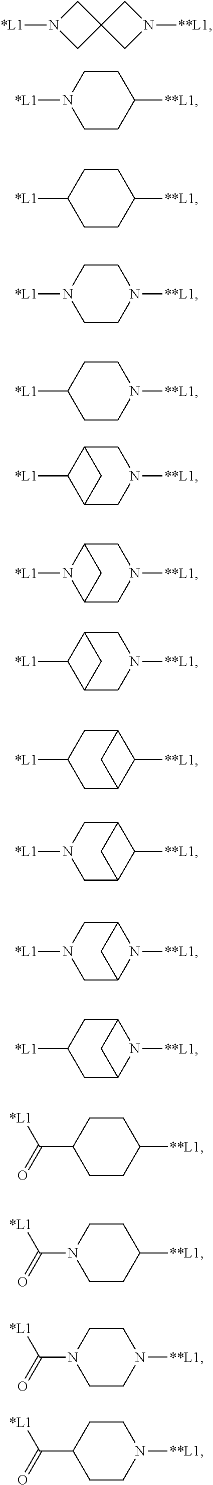

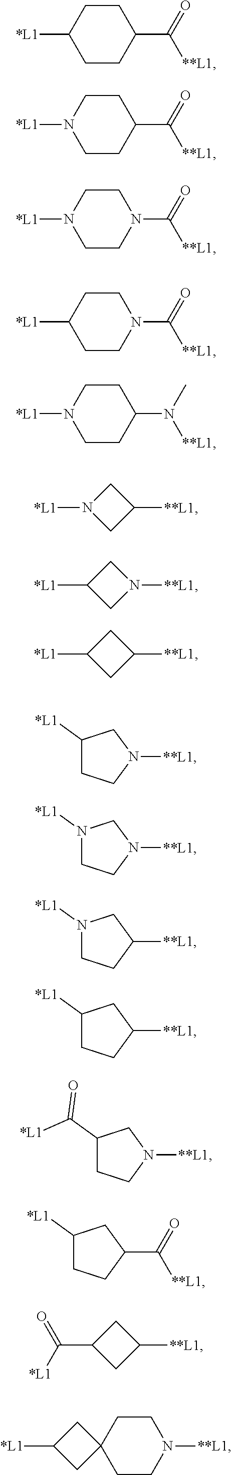

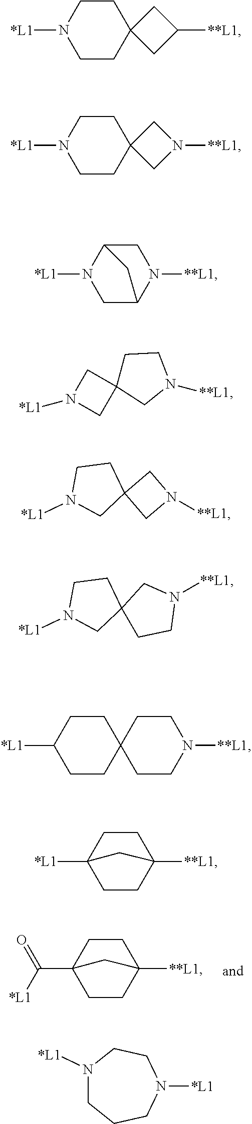

is optionally substituted with at least one R^{L1c};

each of said R^{L1c} is independently selected from oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or

two R^{L1c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl.

Aspect 22. The compound of any one of Aspects 1-21, wherein L¹ is selected from a single bond, —C₁-C₈alkylene-(preferably —CH₂—, —C₂H₄—, —C₃H₆—, —C(O)—, —O—, —N(CH₃)—, —NH—,

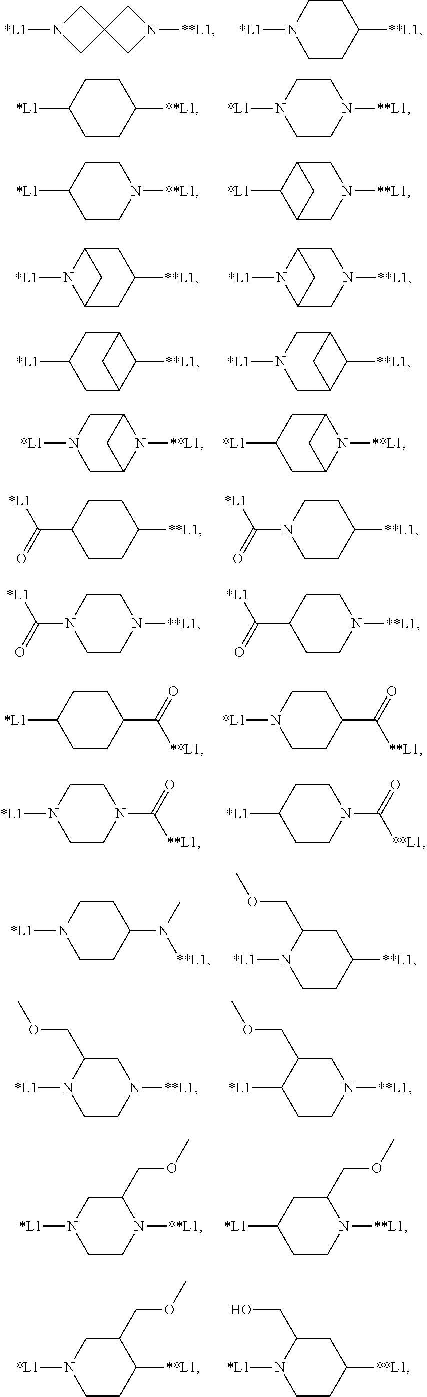

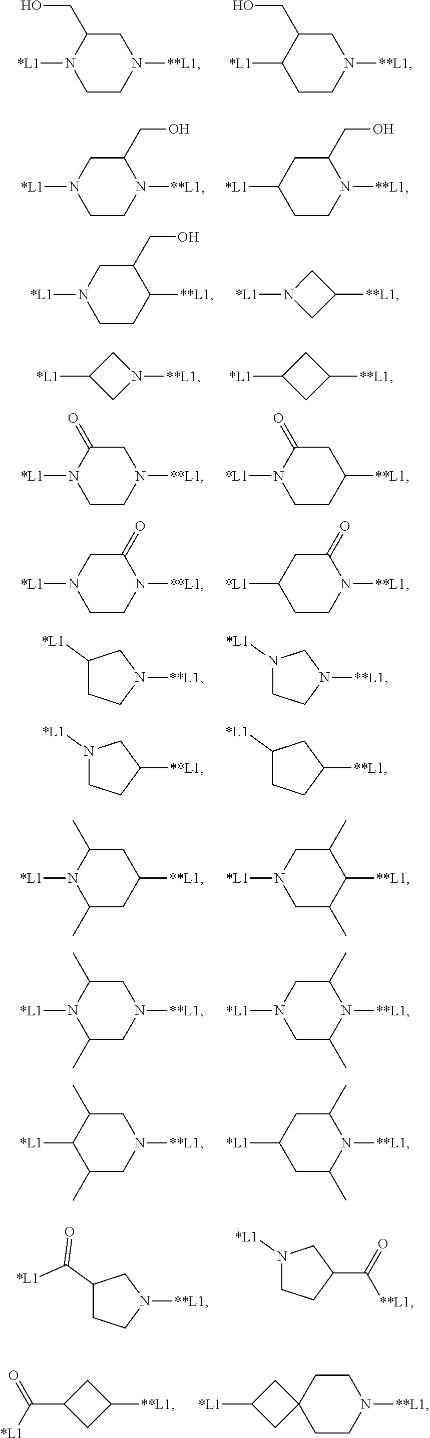

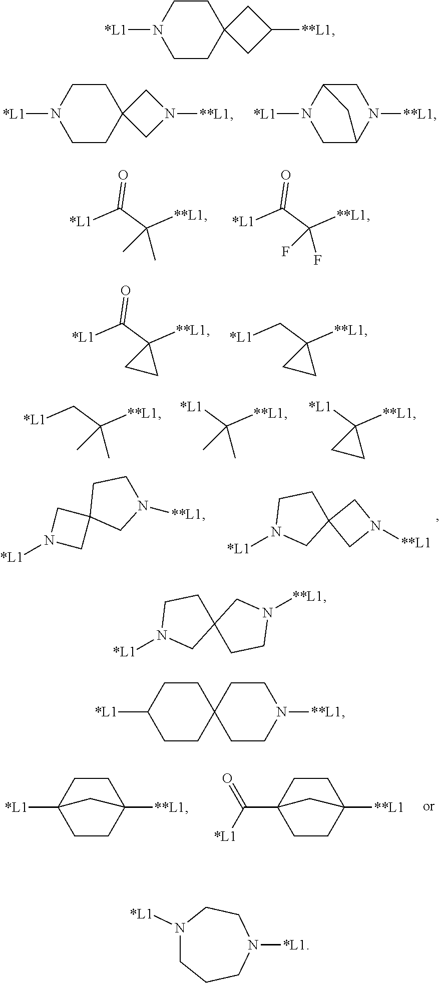

Aspect 23. The compound of any one of Aspects 1-22, wherein X¹ and X² are each independently selected from —CR^{a} or N;

R^{a} is selected from hydrogen, —F, —Cl, —Br, —I, CN, methyl, ethyl, methoxy, ethoxy, cyclopropyl, each of said methyl, ethyl, methoxy, ethoxy, cyclopropyl, is optionally substituted with at least one substituent —F, —Cl, —Br, —I, hydroxy, methyl, ethyl, (preferably, X¹ and X² are each independently selected from CH, C(F), C(CH₃) or N);

m1=1 or 0;

R¹² is hydrogen, oxo (═O), methoxymethyl, hydroxymethyl, —CN or —CH₃.

Aspect 24. The compound of any one of Aspects 1-23, wherein m1 is 1; preferably,

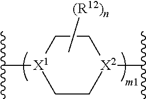

moiety is

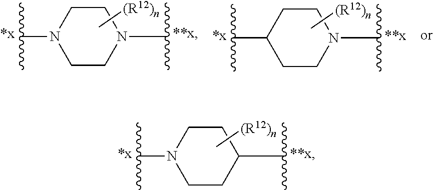

wherein *^{X} refers to the position attached to

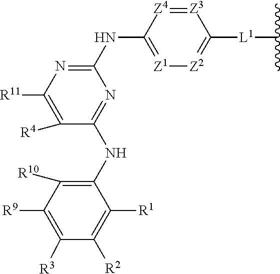

moiety, and **^{X} refers to the position attached to the

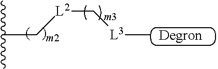

moiety.

Aspect 25. The compound of any one of Aspects 1-24, wherein m1 is 1; preferably,

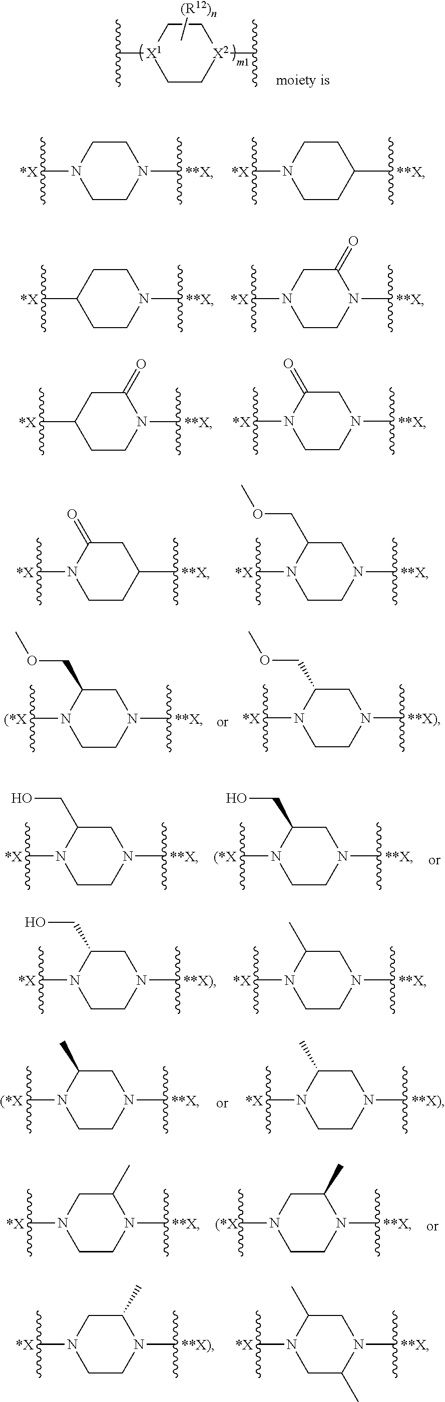

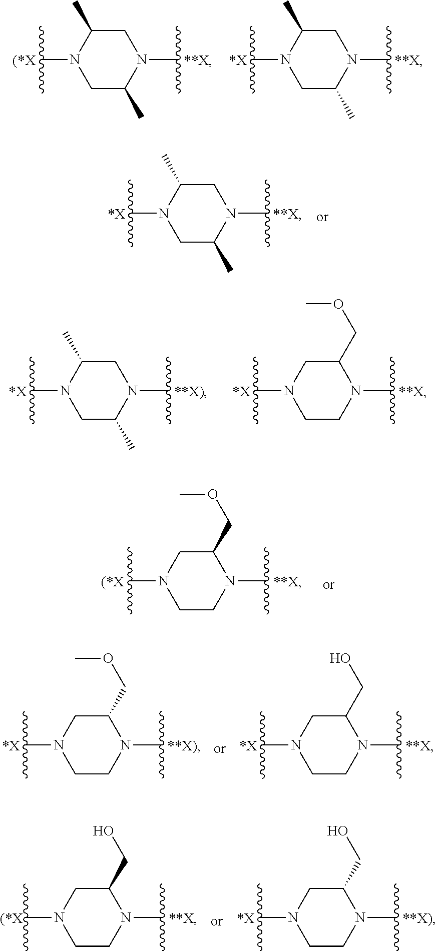

wherein *^{X} refers to the position attached to

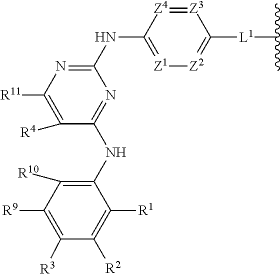

moiety, and **^{X} refers to the position attached to the

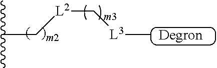

moiety.

Aspect 26. The compound of any one of Aspects 1-25, wherein m1 is 1,

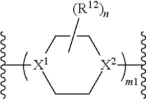

moiety is

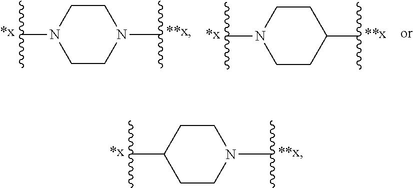 [SMILES: *N1CCN(*)CC1 ; *C1CCN(*)CC1 ; *C1CCN(*)CC1]

wherein *^{X}refers to the position attached to

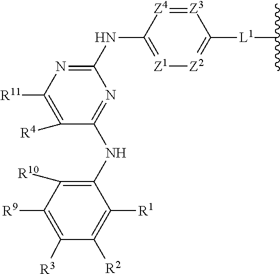

moiety, and **^{X} refers to the position attached to the

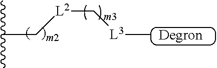

moiety.

Aspect 27. The compound of any one of Aspects 1-26, wherein L² is selected from a single bond, —C₁-C₈alkylene-(preferably —CH₂—, —C₂H₄—, —C₃H₆—), *^{L2}—C(O)—C₁-C₈alkylene_**^{L2} (preferably *^{L2}—C(O)—CH₂—**^{L2}, *^{L2}—C(O)—C₂H₄—**^{L2}, *^{L2}—C(O)—C₃H₆—**^{L2}), *^{L2}—C₁-C₈alkylene-C(O)—**^{L2} (preferably *^{L2}—CH₂—C(O)—**^{L2}, *^{L2} C₂H₄—C(O)—**^{L2}, *^{L2}—C₃H₆—C(O)—**^{L2}), —C(O)—, —O—, —N(CH₃)—, —NH—,

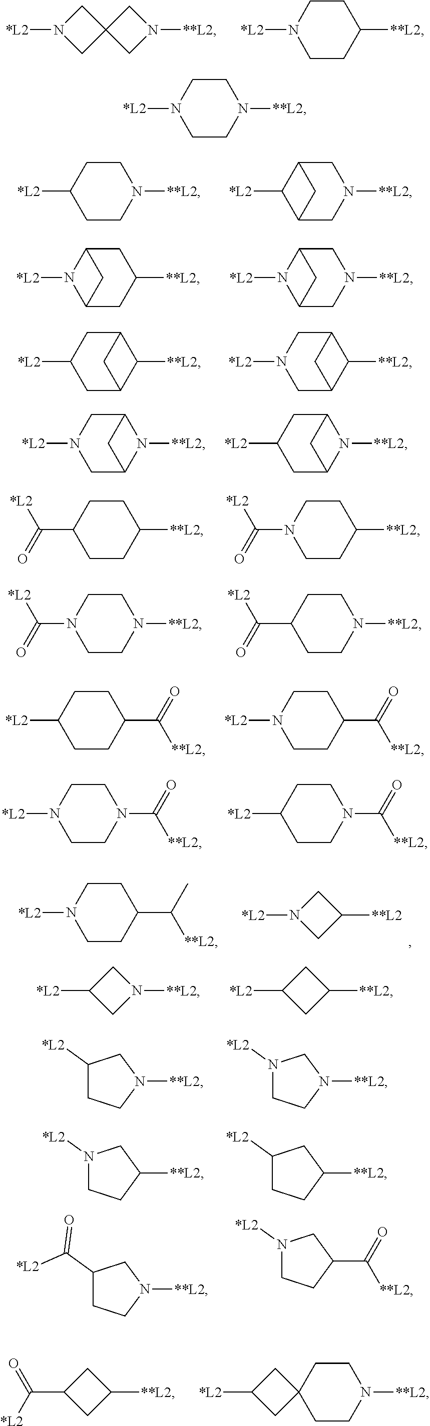

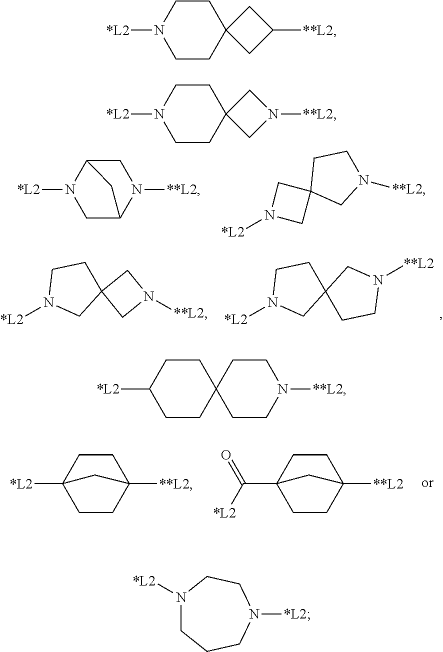

wherein each of said —C₁-C₈alkylene-(preferably —CH₂—, —C₂H₄—, —C₃H₆—), *^{L2}—C(O)—C₁-C₈alkylene-**^{L2} (preferably *^{L2}—C(O)—CH₂—**^{L2}, *^{L2}—C(O)—C₂H₄—**^{L2}, *^{L2}—C(O)—C₃H₆—**^{L2}), *^{L2}—C, —C₈alkylene-C(O)—**^{L2} (preferably *^{L2}—CH₂—C(O)—**^{L2}, *^{L2}—C₂H₄—C(O)—**^{L2}, *^{L2}—C₃H₆—C(O)—**^{L2}), —N(CH₃)—, —NH—,

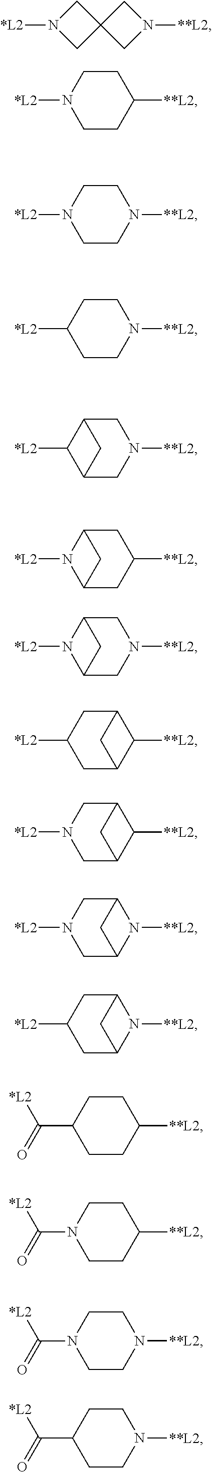

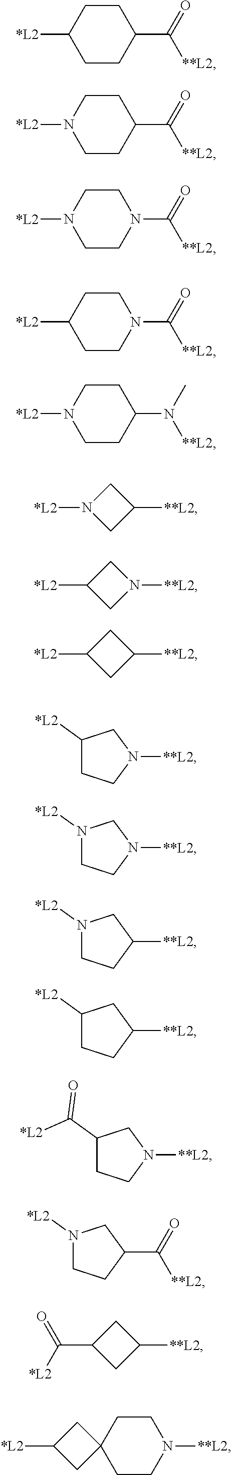

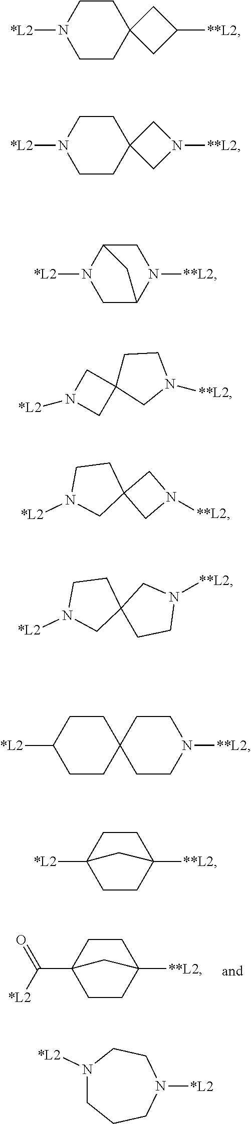

is optionally substituted with at least one R^{L2c};

each of said R^{L2c} is independently selected from oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or two R^{L2c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, or octyl.

Aspect 28. The compound of any one of Aspects 1-27, wherein L² is selected from a single bond, —C₁-C₈alkylene-(preferably —CH₂—, —C₂H₄—, —C₃H₆—), —C(O)—, —O—, —N(CH₃)—, —NH—,

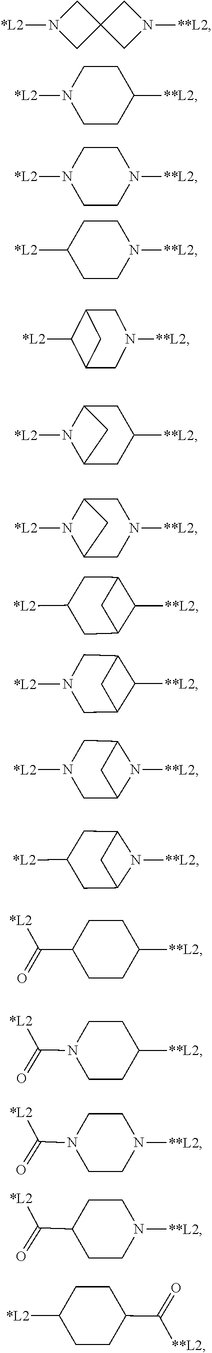

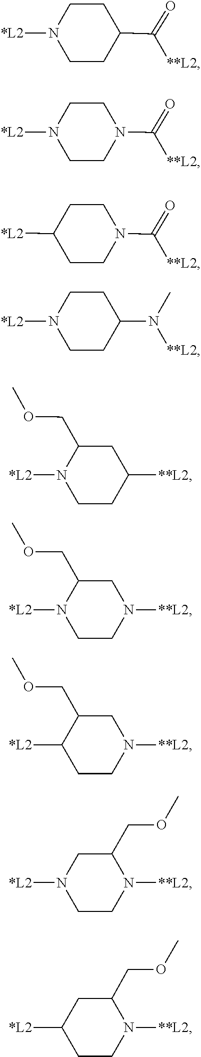

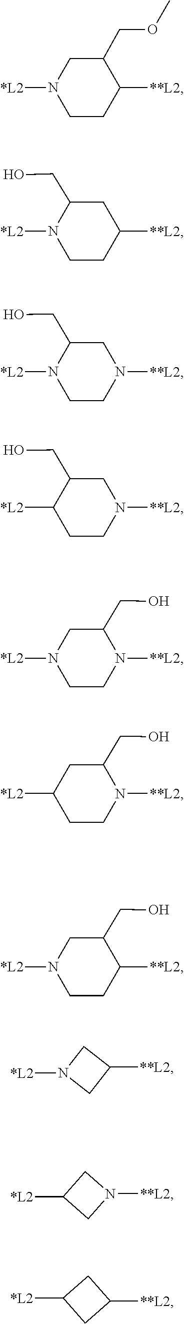

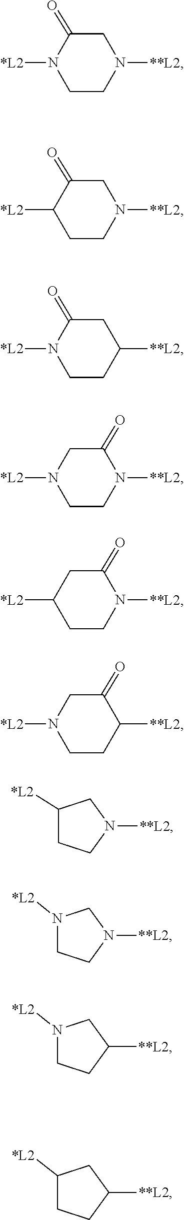

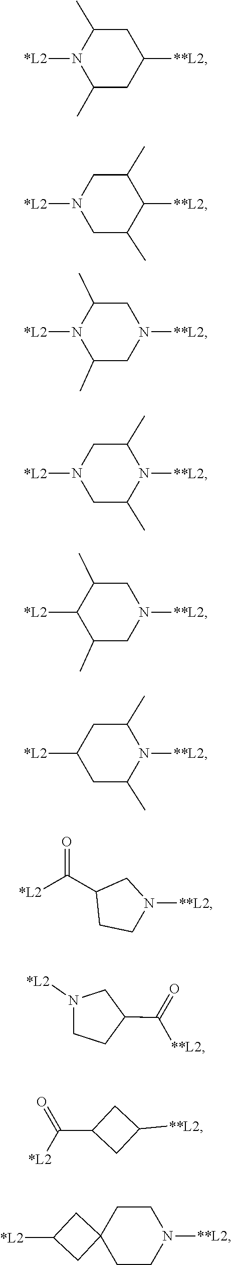

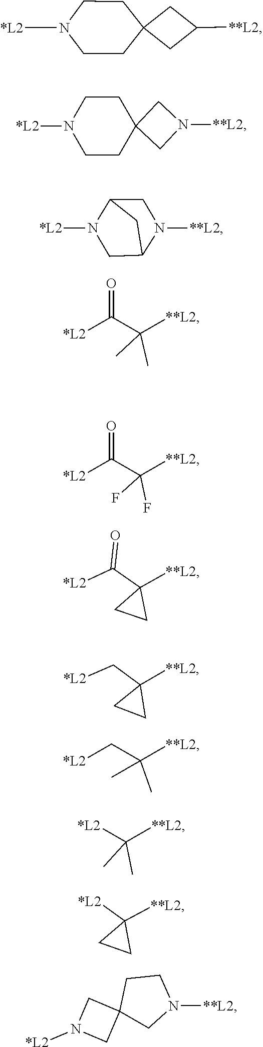

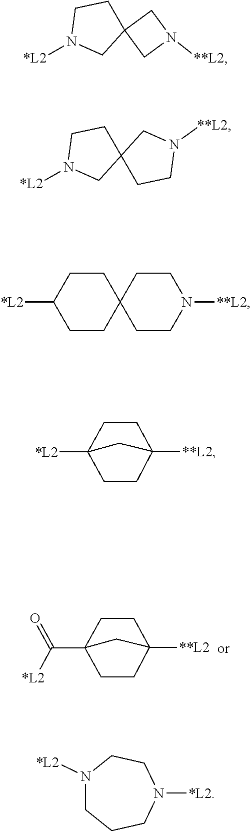

Aspect 29. The compound of any one of Aspects 1-28, wherein L³ is selected from single bond, —C₁-C₈alkylene-(preferably —CH₂—, —C₂H₄—, —C₃H₆—), *^{L3}—C(O)—C₁-C₈alkylene-**^{L3} (preferably *^{L3}—C(O)—CH₂—**^{L3}*^{L3}—C(O)—C₂H₄—**^{L3}, *^{L3}—C(O)—C₃H₆—**L¹³), *^{L3}—C₁-C₈alkylene-C(O)—**^{L3} (preferably *^{L3}—CH₂—C(Q)-**^{L3}, *^{L3}-C₂H₄—C(O)—**^{L3}, *^{L3}—C₃H₆—C(O)—**^{L3}), —C(O)—, —O—, —N(CH₃)—, —NH—,

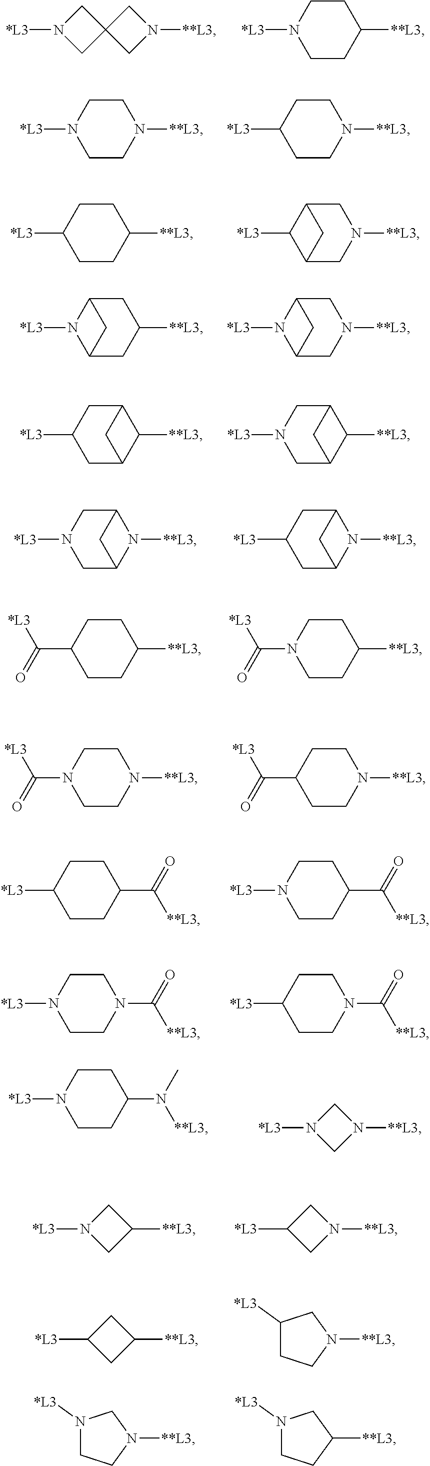

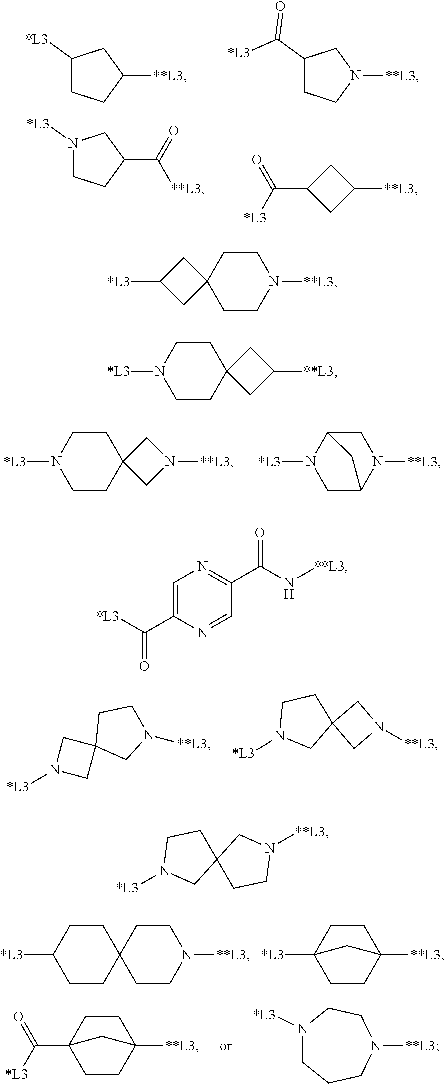

wherein each of said —C₁-C₈alkylene-(preferably —CH₂—, —C₂H₄—, —C₃H₆—), *^{L3}—C(O)—C₁-C₈alkylene_**^{L3} (preferably *^{L3}—C(O)—CH₂—**^{L3}, *^{L3}—C(O)—C₂H₄—**^{L3}, *^{L3}—C(O)—C₃H₆—**^{L3}, *^{L3}—C₁-C₈alkylene-C(O)—**^{L3} (preferably *^{L3}—CH₂—C(O)—**^{L3}, *^{L3}—C₂H₄—C(O)—**^{L3}, *^{L3}—C₃H₆—C(O)—**^{L3}), —N(CH₃)—, —NH—,

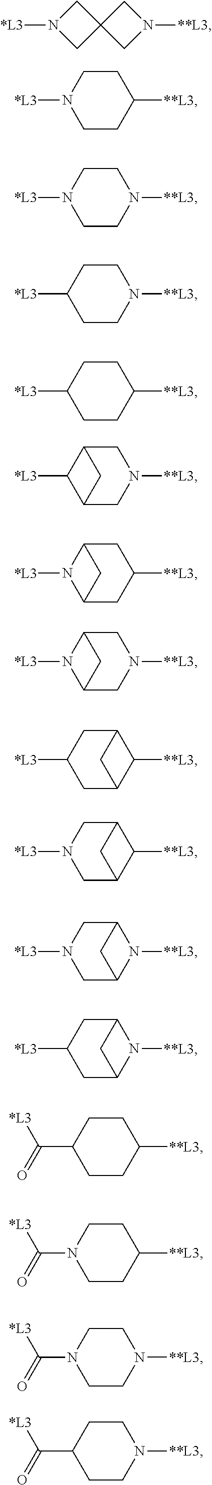

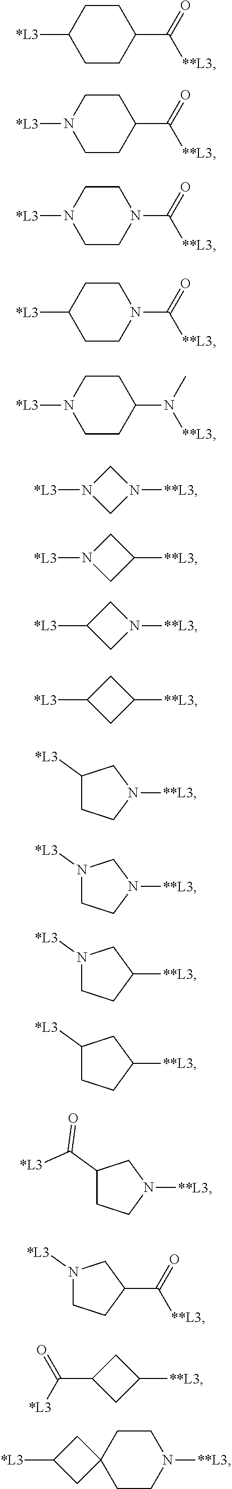

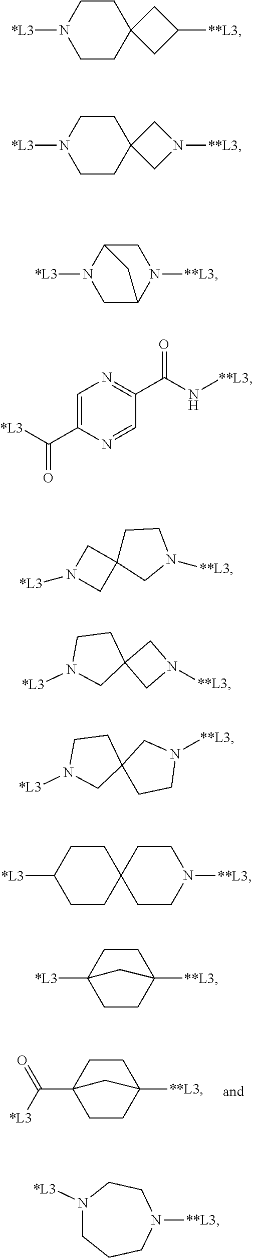

is optionally substituted with at least one R^{L3c};

each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or

two R^{L3c} together with the carbon atoms to which they are attached, form a 3-, 4-, 5-, 6-, 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl.

Aspect 30. The compound of any one of Aspects 1-29, wherein L³ is selected from a single bond, —C₁-C₈alkylene-(preferably —CH₂—, —C₂H₄—, —C₃H₆—), —C(O)—, —O—, —N(CH₃)—, —NH—,

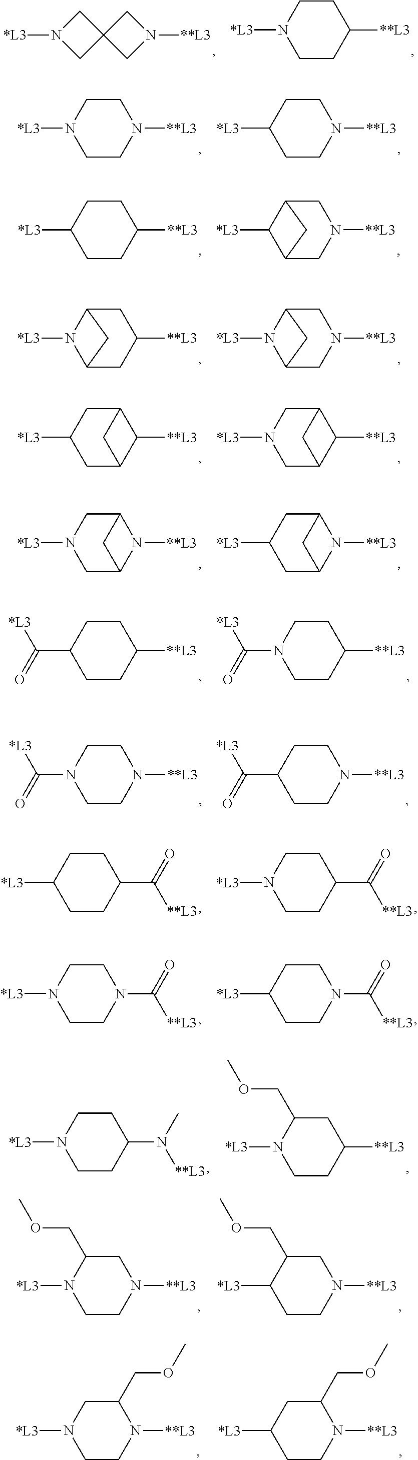

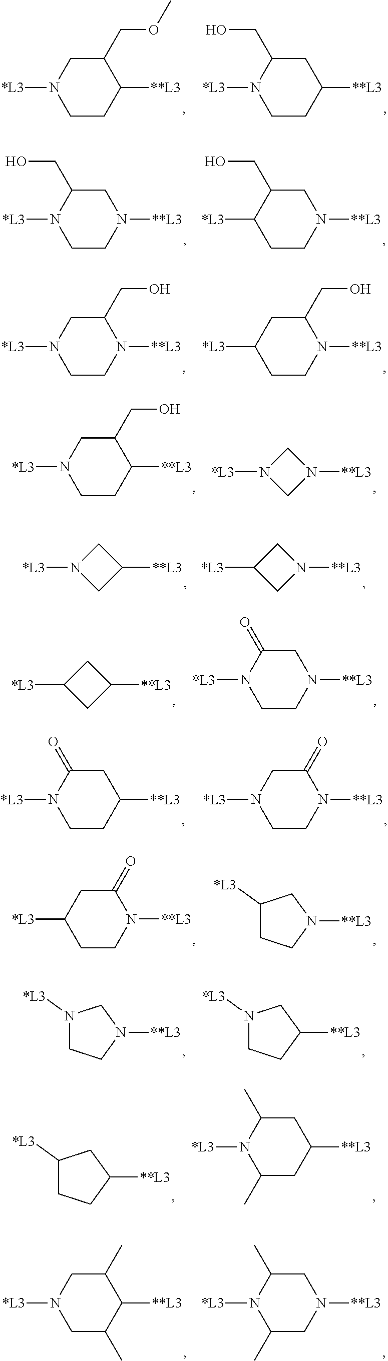

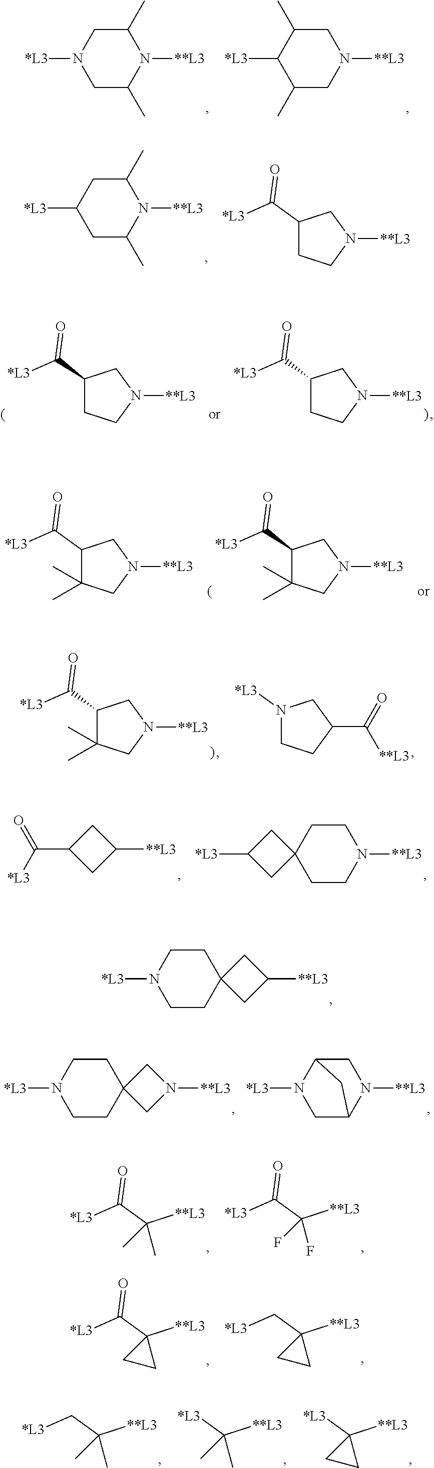

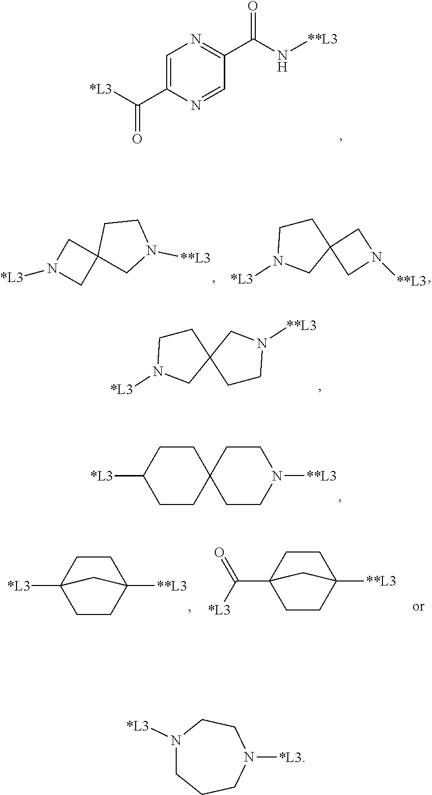

Aspect 31. The compound of any one of Aspects 1-30, wherein L² is a single bond, L³ is a single bond, or L² and L3 are both single bond.

Aspect 32. The compound of any one of Aspects 1-31, wherein

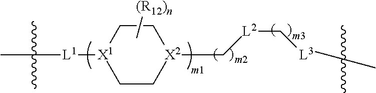

is selected from

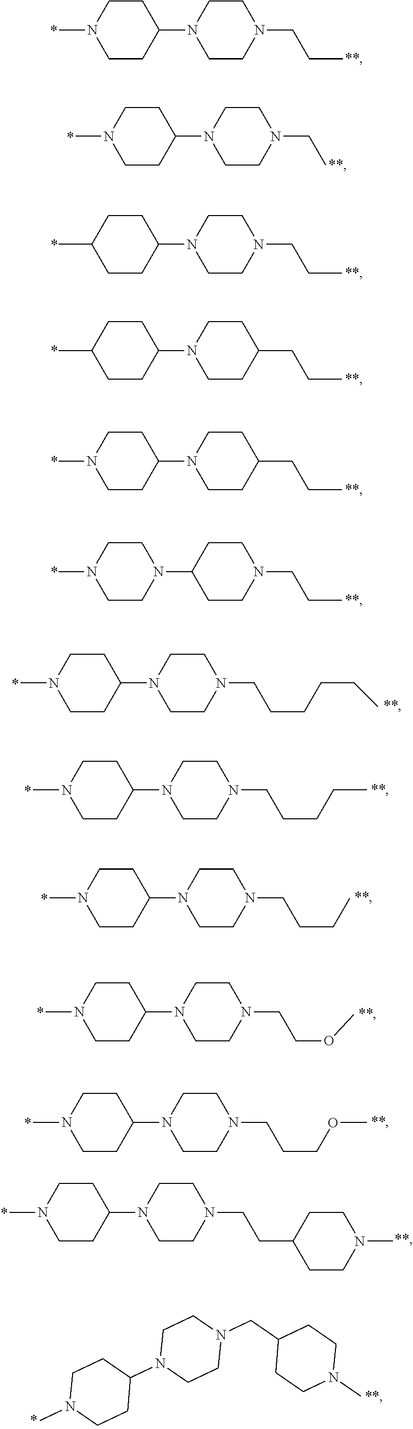

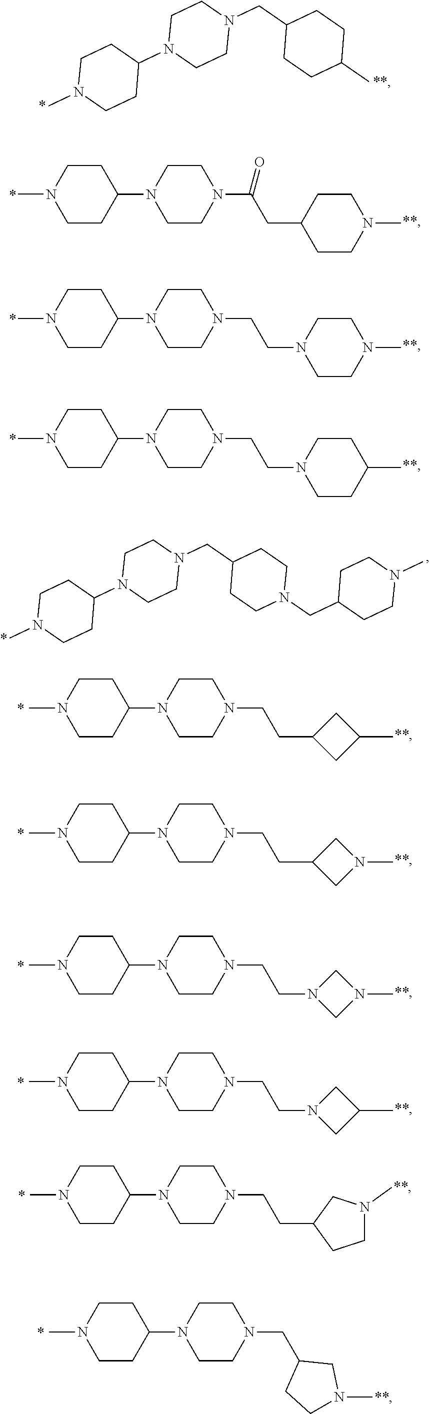

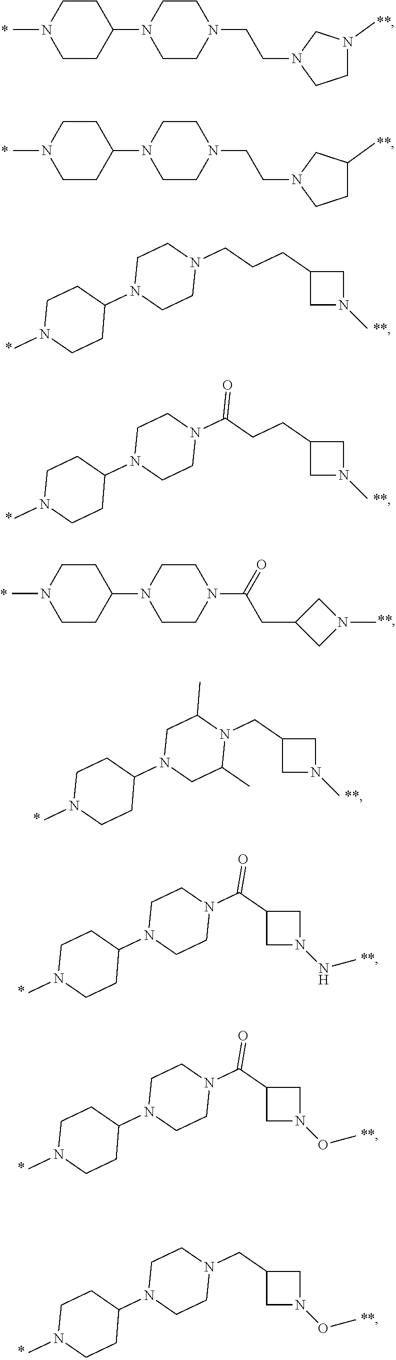

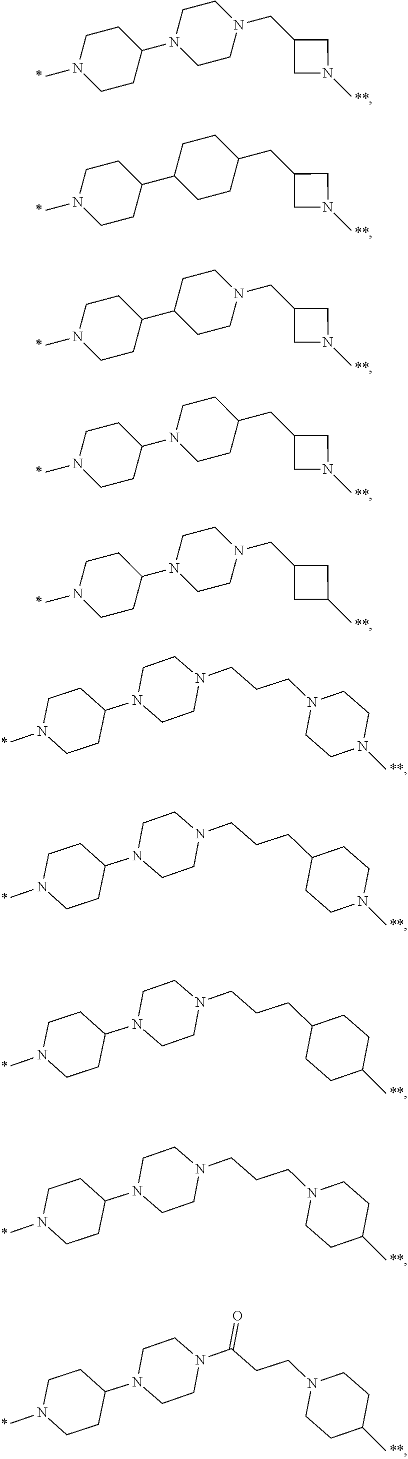

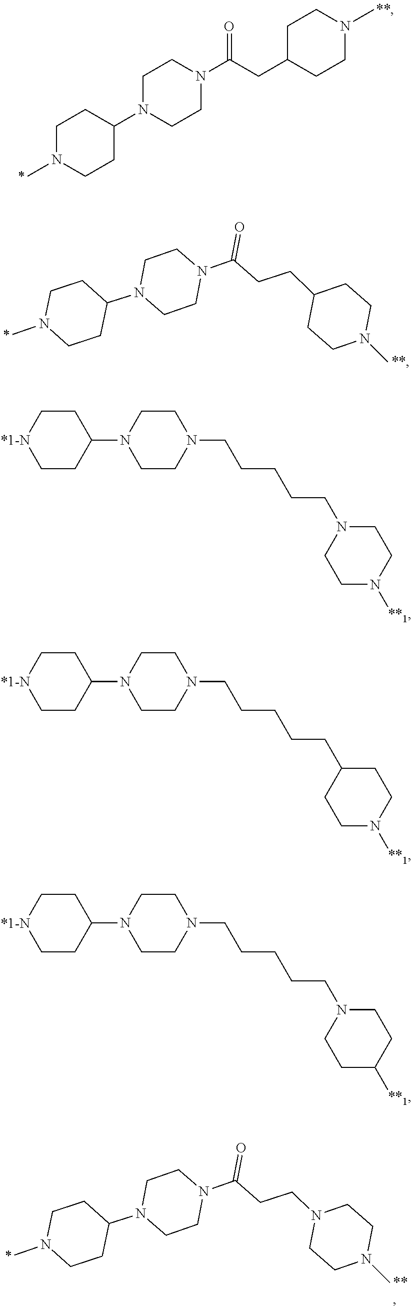

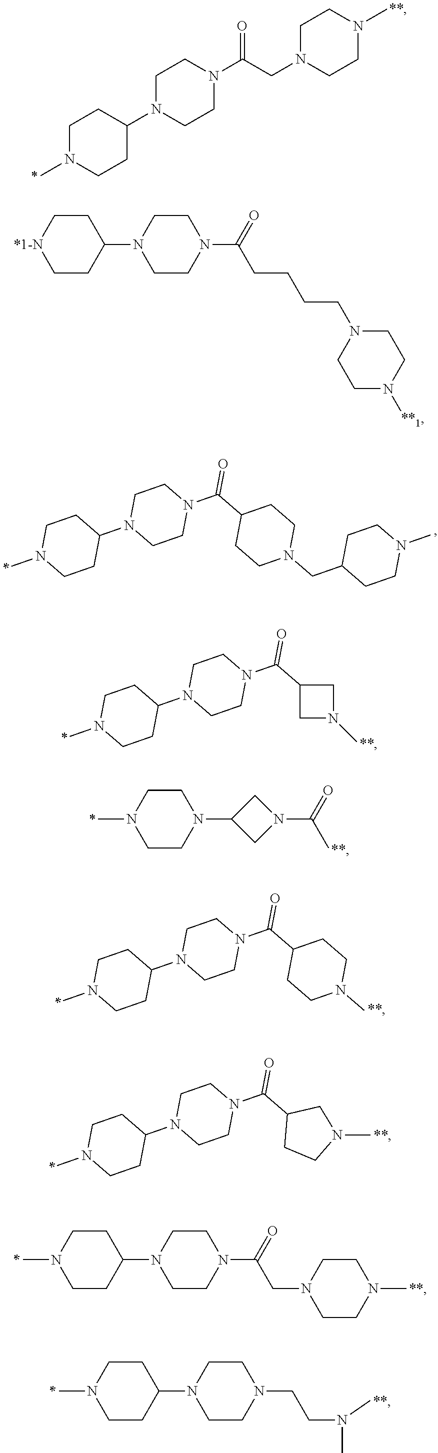

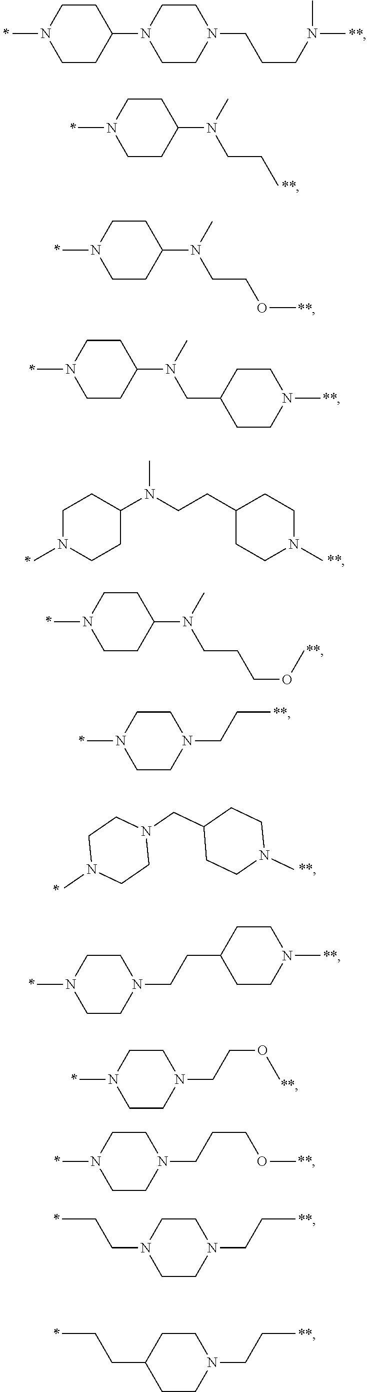

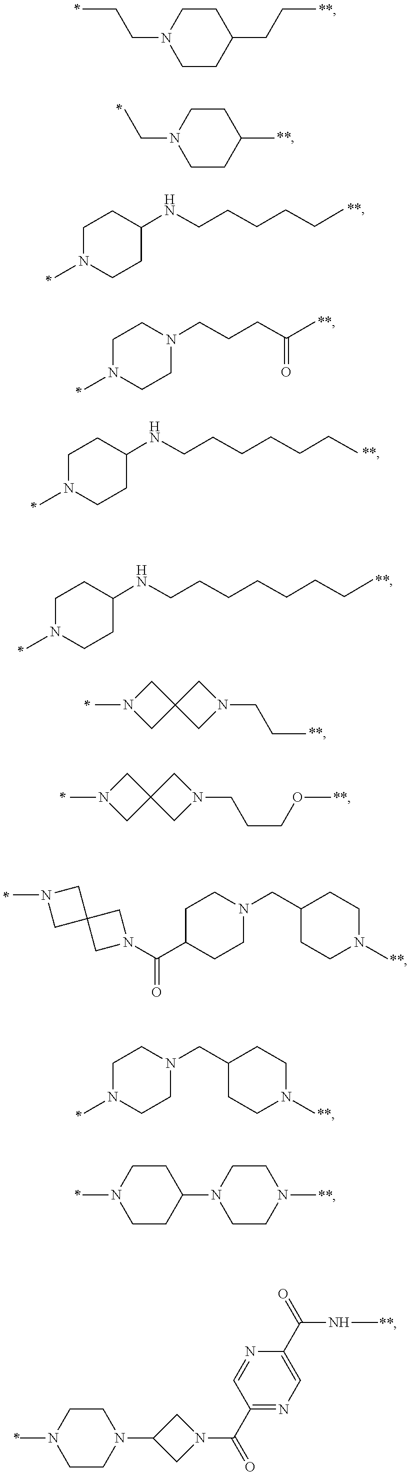

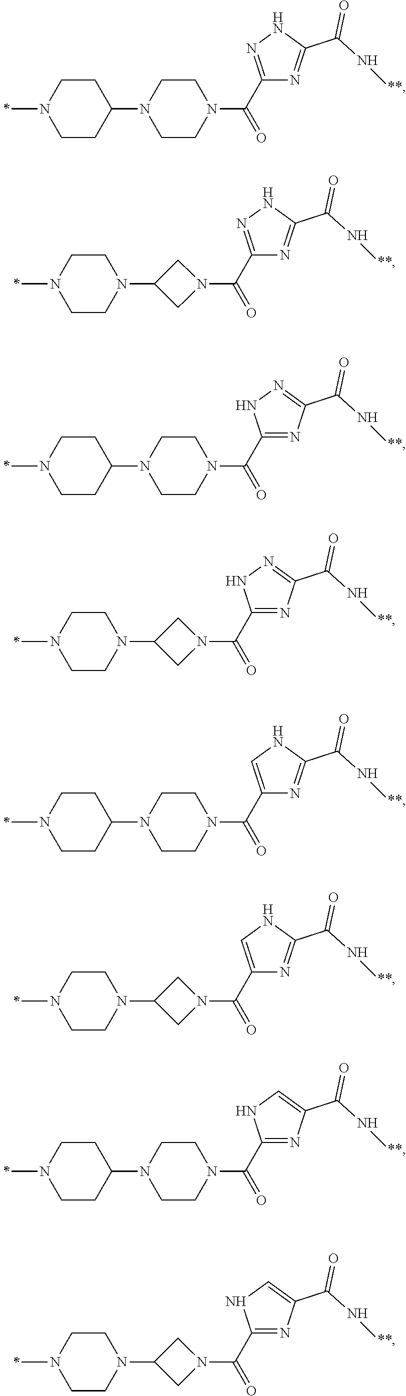

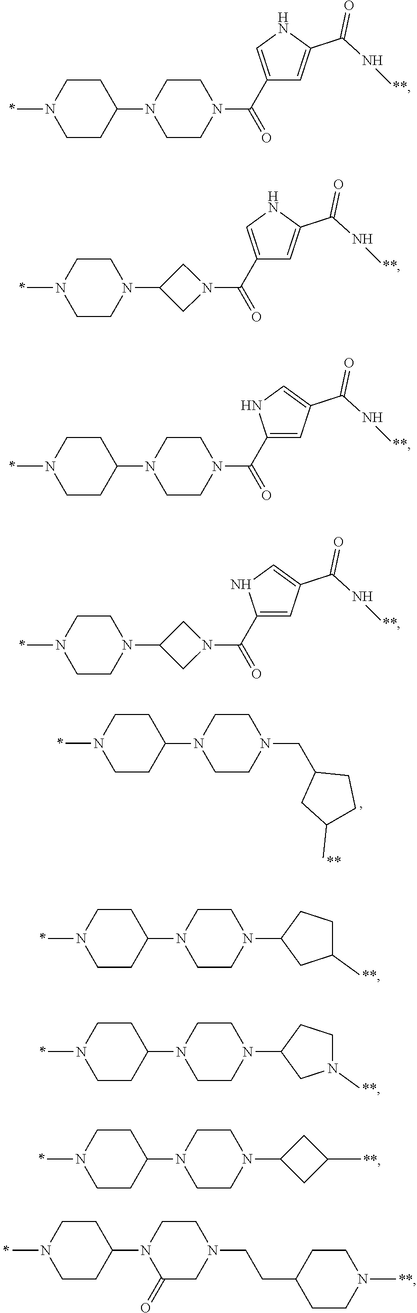

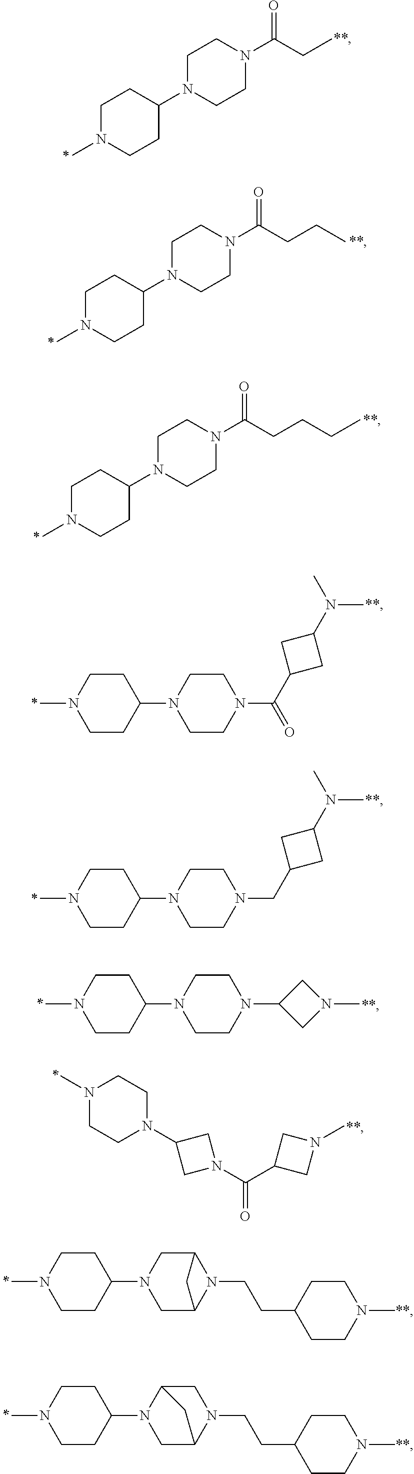

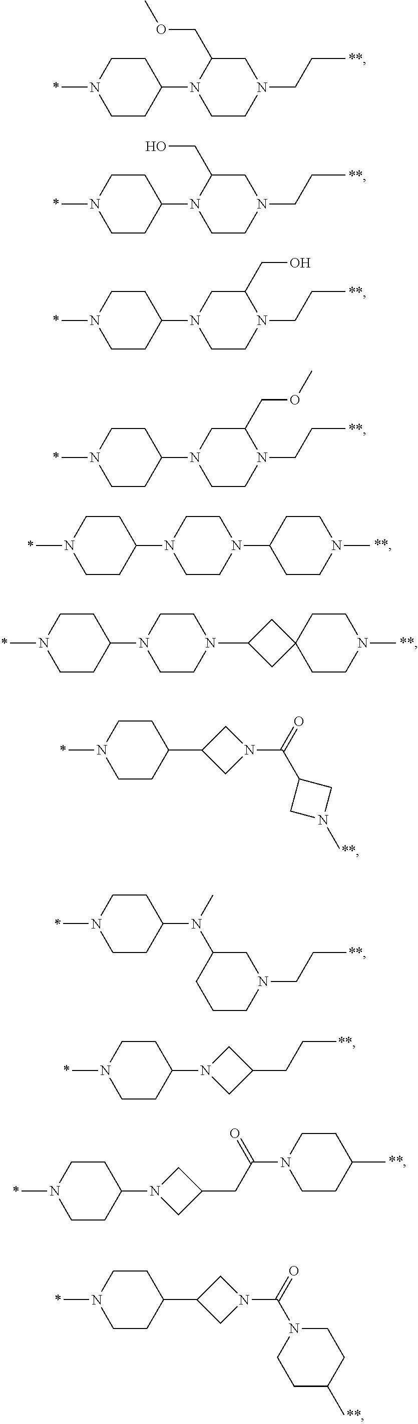

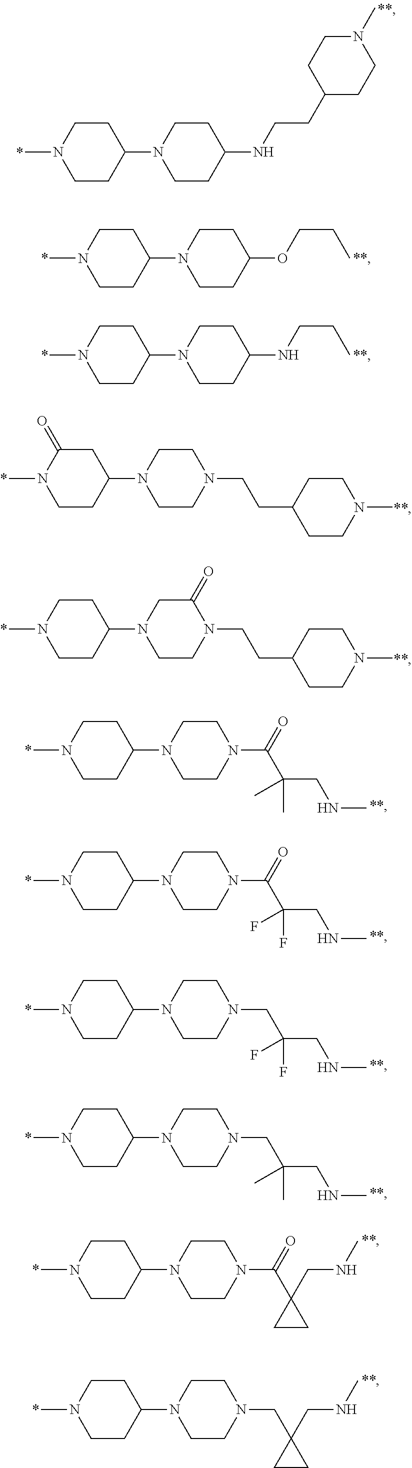

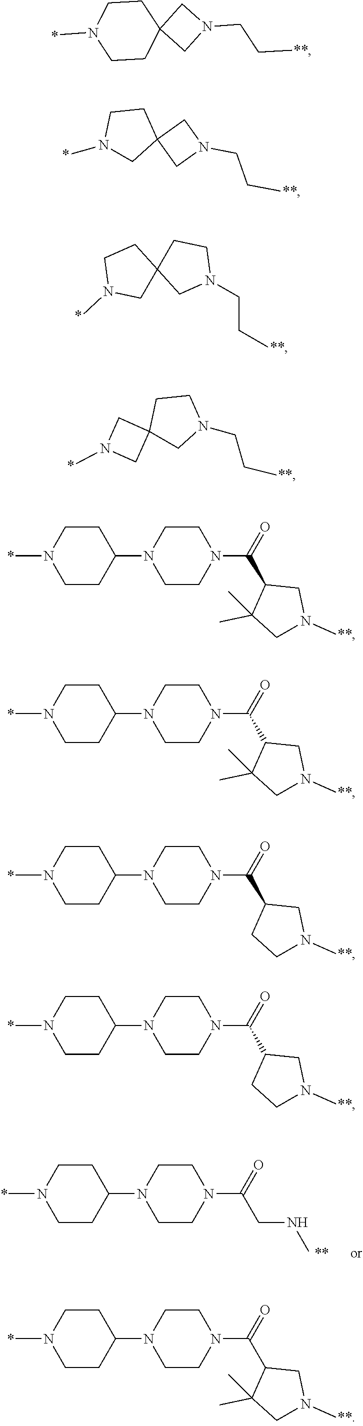

Aspect 33. The compound of any one of Aspects 1-32, wherein R¹³, R¹⁴, R¹⁵, R¹⁶ and R¹⁷ are each independently selected from hydrogen, —F, —Cl, —Br, —I, CN, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, 15 cyclobutyl, cyclopentyl, cyclohexyl, cycloheptyl, cyclooctyl, 3- to 8-membered heterocyclyl, phenyl or 5- to 12-membered heteroaryl; said each methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent —F, —Cl, —Br, —I, methyl, ethyl, propyl, butyl, pentyl, hexyl, heptyl, octyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 34. The compound of any one of Aspects 1-33, wherein at each occurrence, R^{a} and R^{b} are each independently selected from hydrogen, —F, —Cl, —Br, —I, CN, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl; or

R^{a} and R^{b} together with the carbon atoms to which they are attached, form a 3, 4, 5, 6, 7 or 8-membered ring, said ring comprising 0-3 heteroatoms independently selected from nitrogen, oxygen or sulfur; said ring is optionally substituted with at least one substituent —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl.

Aspect 35. The compound of any one of Aspects 1-34, wherein

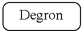

is selected from

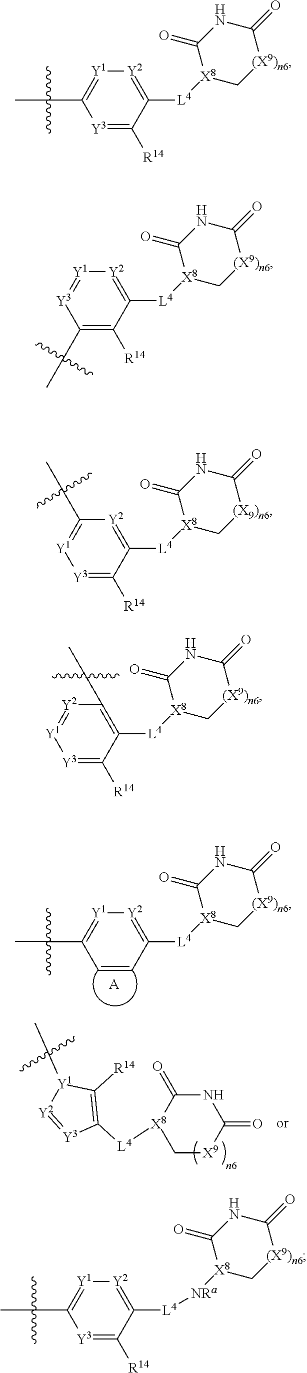

Ring A is selected from 5- to 7-membered cycloalkyl, 5- to 7-membered heterocyclyl, aryl, or heteroaryl;

R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen or —C₁-C₈alkyl; preferably R¹⁴ is independently selected from H, F, Cl, Br, I, CH₃, —OCH₃, —OCH₂F, —OCHF₂, —O CF₃, CH₂F, CN, CHF₂, or CF₃;

X⁸ is independently selected from CH, CD, C(CH₃), C(C₂H₅), C(C₃H₇), C(F) or N;

L⁴ is independently selected from a single bond, —O—, —NH—, —CH₂—, —CHF—, or —CF₂—;

Y¹, Y², and Y³ are each independently selected from CR^{a} or N;

X⁹ is CH₂;

R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen, hydroxy, —C₁-C₈alkyl, or —C₁-C₈alkoxy; and

n6 is independently 0,1 or 2.

Aspect 36. The compound of any one of Aspects 1-35, wherein

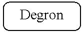

is selected from

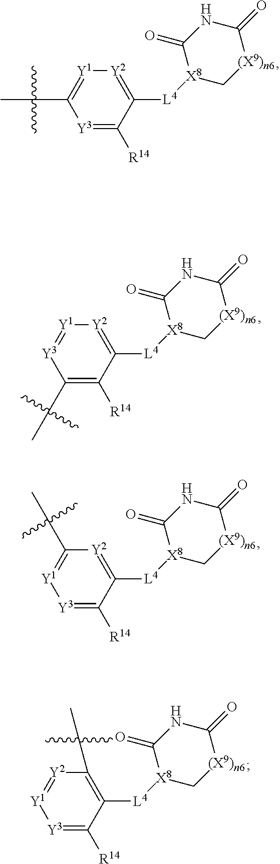

R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen; preferably R¹⁴ is independently selected from H, F, Cl, Br, I, CH₃, —OCH₃, —OCH₂F, —OCHF₂, —O CF₃, CH₂F, CN, CHF₂, or CF₃;

X⁸ is independently selected from CH, CD, C(CH₃), C(C₂H₅), C(C₃H₇), C(F) or N;

L⁴ is a single bond;

Y¹, Y², and Y³ are each independently selected from CR^{a} or N;

X⁹ is CH₂;

R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen; and

n₆ is 1.

Aspect 37. The compound of any one of Aspects 1-36, wherein

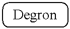

is selected from

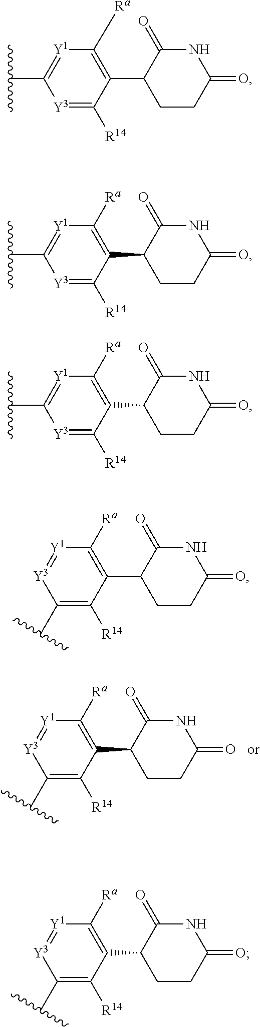

R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen; preferably R¹⁴ is independently selected from H, F, Cl, Br, I, CH₃, —OCH₃, —OCH₂F, —OCHF₂, —O CF₃, CH₂F, CN, CHF₂, or CF₃;

Y¹ and Y³ are each independently selected from CH or N;

R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen.

Aspect 38. The compound of any one of Aspects 1-35, wherein

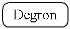

is selected from

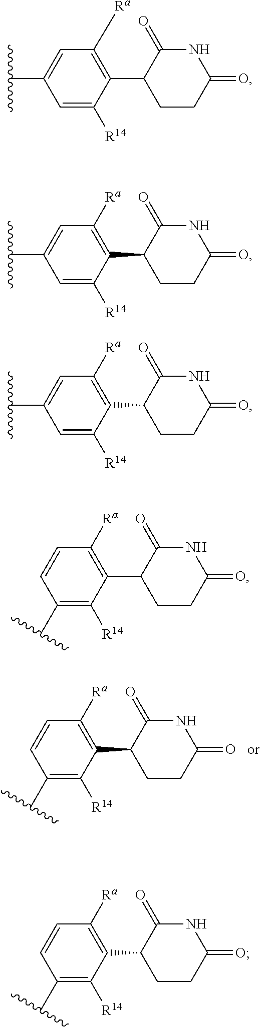

R¹⁴ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, or CN; said each methyl, ethyl, propyl, butyl, pentyl, hexyl, heptyl, octyl, methoxy, ethoxy, propoxy, butoxy, pentoxy, hexoxy, heptyloxy, octyloxy is optionally substituted by one or more F, Cl, Br, I; preferably R¹⁴ is independently selected from H, F, Cl, Br, I, CH₃, —OCH₃, —OCH₂F, —OCHF₂, —O CF₃, CH₂F, CN, CHF₂, or CF₃;

R^{a} is each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, each of said methyl, ethyl, propyl, butyl, pentyl, hexyl, heptyl, octyl, methoxy, ethoxy, propoxy, butoxy, pentoxy, hexoxy, heptyloxy, octyloxy is optionally substituted with at least one or more F, Cl, Br, I.

Aspect 39. The compound of any one of Aspects 1-38, wherein

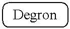

is selected from

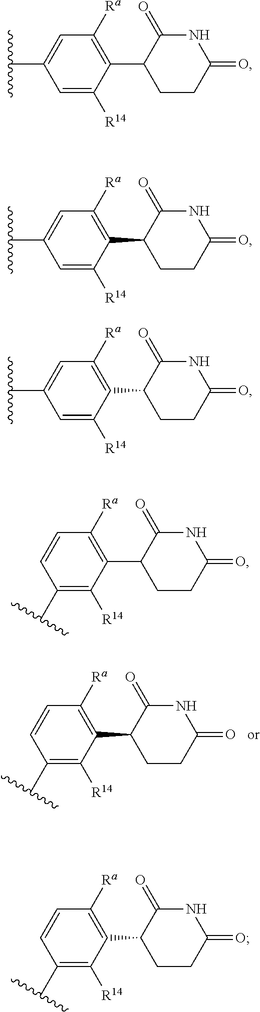

R¹⁴ is independently selected from F, Cl, Br, I, —CH₃, —OCH₃, —OCH₂F, —OCHF₂, —O CF₃, CH₂F, CN, CHF₂, or CF₃;

R^{a} is each independently selected from F, Cl, Br, I, —CH₃, —OCH₃, —OCH₂F, —OCHF₂, —O CF₃, CH₂F, CN, CHF₂, or CF₃.

Aspect 40. The compound of any one of Aspects 1-39, wherein

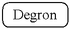

is

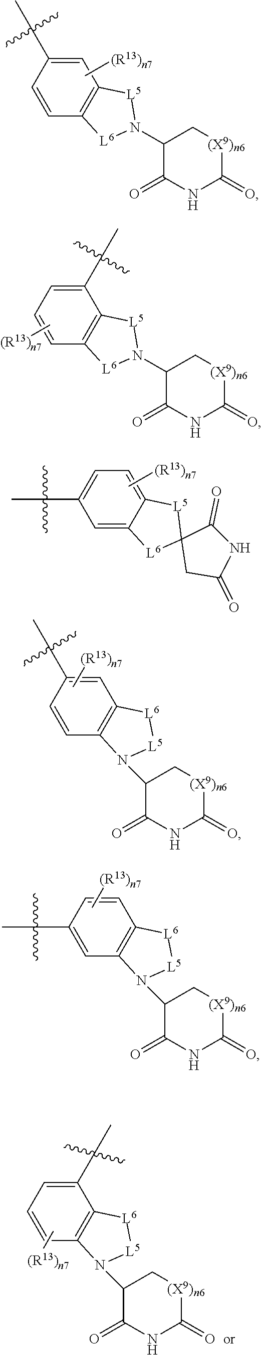

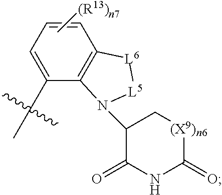

Wherein L⁵ and L⁶ are independently selected from a single bond, —O—, —NR^{a}—, —(CR^{a}R^{b})n₈-, —O(CR^{a}R^{b})n₈-, —NR^{a}(CR^{a}R^{b})n₈- or —C(O)—;

X⁹ is —CR^{a}R^{b}—;

R^{a} and R^{b} are each independently selected from hydrogen, hydroxy, F, Cl, Br, I, CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl and 5- to 12-membered heteroaryl is optionally substituted with at least one substituent halogen, hydroxy, F, Cl, Br, I, CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl; or

R^{a} and R^{b} together with the carbon atoms to which they are attached, form a 3-, 4-, 5-, 6-, 7-, 8-membered ring, said ring comprising 0-3 heteroatoms independently selected from nitrogen, oxygen or sulfur; said ring is optionally substituted with at least one substituent halogen, hydroxy, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;

each R¹³ is independently selected from hydrogen, halogen, CN, —C₁-C₈alkyl, or —C₁-C₈alkoxy;

n₆ is 0 or 1; and

n₇ is 0, 1 or 2.

Aspect 41. The compound of any one of Aspects 1-40, wherein

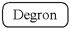

is

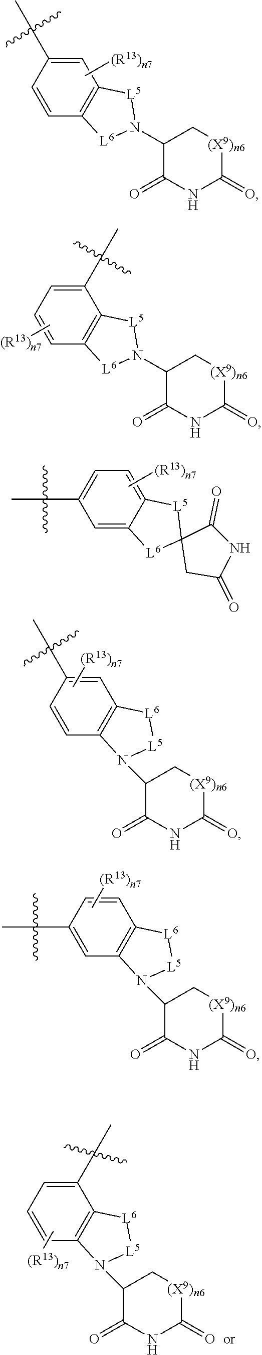

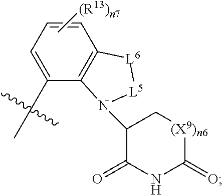

Wherein L⁵ and L⁶ is independently selected from a single bond,

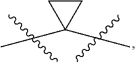 [SMILES: *C1(*)CC1]

—O—, —NH—, —NMe-, —N(CH₂CH₃)—, —N(ⁱPr)—, —CH₂—, —CHF—, —CF₂—, —C(CH₃)₂— or —C(O)— (preferably L⁵ is —C(O)— or —CH₂—, and L⁶ is

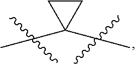 [SMILES: *C1(*)CC1]

—O—, —NH—, —NMe-, —N(CH₂CH₃)—, —N(ⁱPr)—,

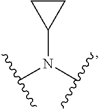 [SMILES: *N(*)C1CC1]

—CH₂—, —CHF—, —CF₂—, —C(CH₃)₂— or —C(O)—);

X⁹ is CH₂;

each R¹³ is independently selected from hydrogen, halogen, CN, —C₁-C₈alkyl, or —C₁-C₈alkoxy;

n₆ is 0 or 1; and

n₇ is 0, 1 or 2.

Aspect 42. The compound of any one of Aspects 1-41, wherein

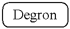

is

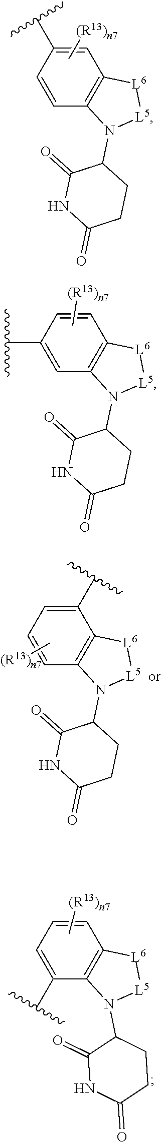

Wherein L⁵ and L⁶ are each independently selected from

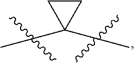 [SMILES: *C1(*)CC1]

—O—, —NH—, —NMe-, —N(CH₂CH₃)—, —N(ⁱPr)—, —CH₂—, —CHF—, —CF₂—, —C(CH₃)₂— or —C(O)—;

each R¹³ is independently selected from hydrogen, F, Cl, Br, I, CN, -Me, -Et, —C₃H₇, —C₄H₉, —OMe, —OCH₂F, —OCHF₂, —O CF₃, —OEt, —OC₃H₇ or —OC₄H₉;

n₇ is 0, 1 or 2.

Aspect 43. The compound of any one of Aspects 1-42, wherein

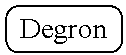

is

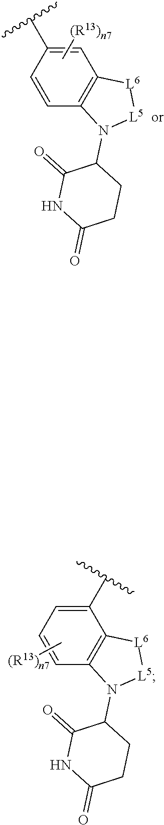

Wherein L⁶ is selected from

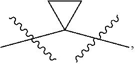 [SMILES: *C1(*)CC1]

—O—, —NMe-, —N(CH₂CH₃)—, —N(ⁱPr)—, —CH₂—, —CHF—, —CF₂— or —C(CH₃)₂—;

Wherein L⁵ is —C(O)—;

each R¹³ is independently selected from hydrogen, F, Cl, Br, I, CN, —C₁-C₈alkyl, or —C₁-C₈alkoxy;

n₇ is 0, 1 or 2.

Aspect 44. The compound of any one of Aspects 1-43, wherein

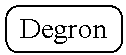

is

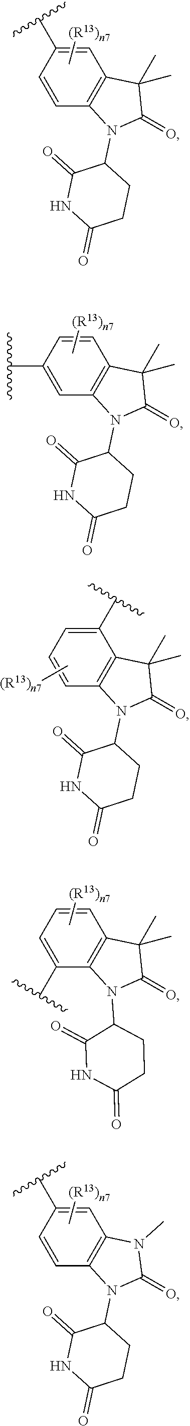

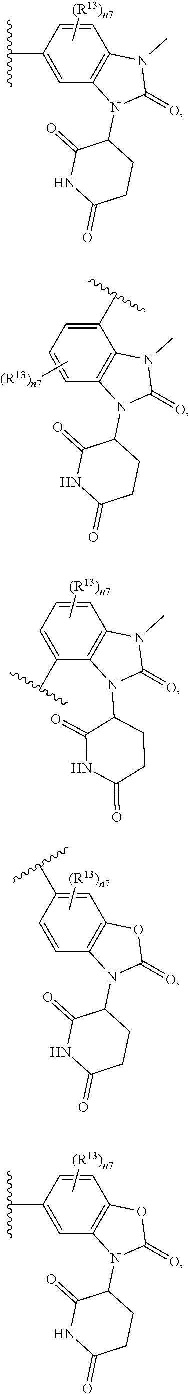

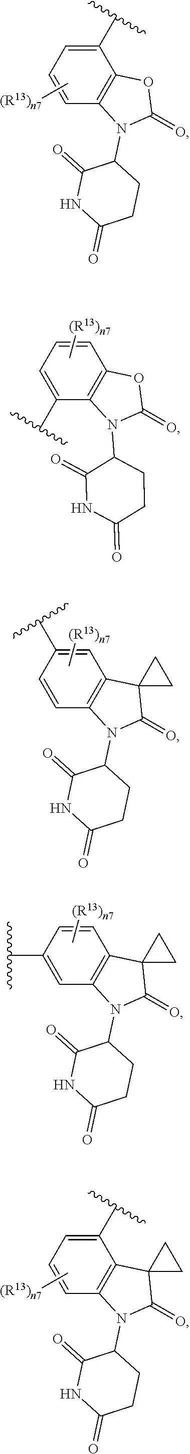

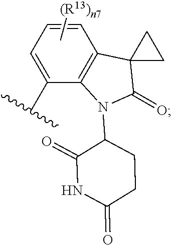

each R¹³ is independently selected from hydrogen, F, Cl, Br, I, CN, -Me, -Et, —C₃H₇, —C₄H₉, —OMe, —OCH₂F, —OCHF₂, —O CF₃, —OEt, —OC₃H₇ or —OC₄H₉;

n₇ is 0, 1 or 2.

Aspect 45. The compound of any one of Aspects 1-44, wherein

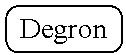

is

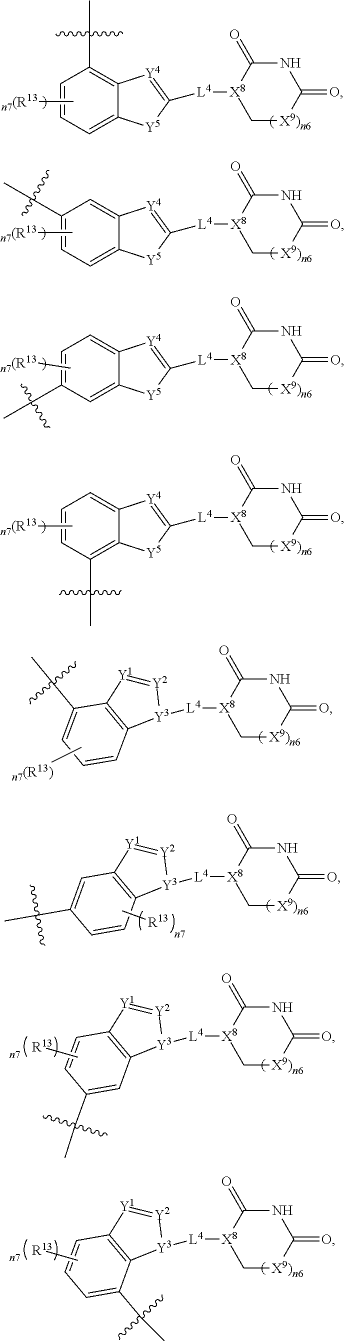

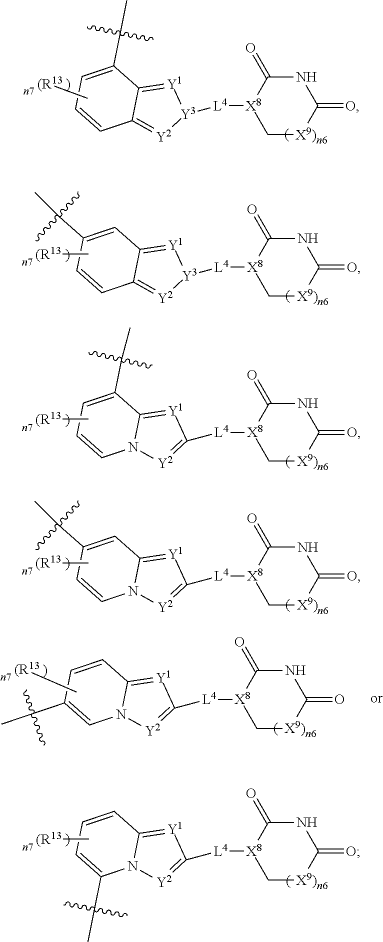

Wherein L⁴ is independently selected from a single bond,

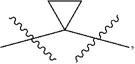 [SMILES: *C1(*)CC1]

—O—, —NH—, —CH₂—, —CHF—, or —CF₂—;

X⁸ is independently selected from CH, C(CH₃), C(C₂H₅), C(C₃H₇), C(F) or N;

X⁹ is CH₂;

each R¹³ is independently selected from hydrogen, halogen, CN, —C₁-C₈alkyl, or —C₁-C₈alkoxy;

Y¹, Y², Y³ and Y⁴ are each independently selected from CH, C(CH₃), C(F), or N;

Y⁵ is selected from NH, N(CH₃), O or S;

n₆ is 0 or 1; and

n₇ is 0, 1 or 2.

Aspect 46. The compound of any one of Aspects 1-45, wherein

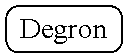

is selected from

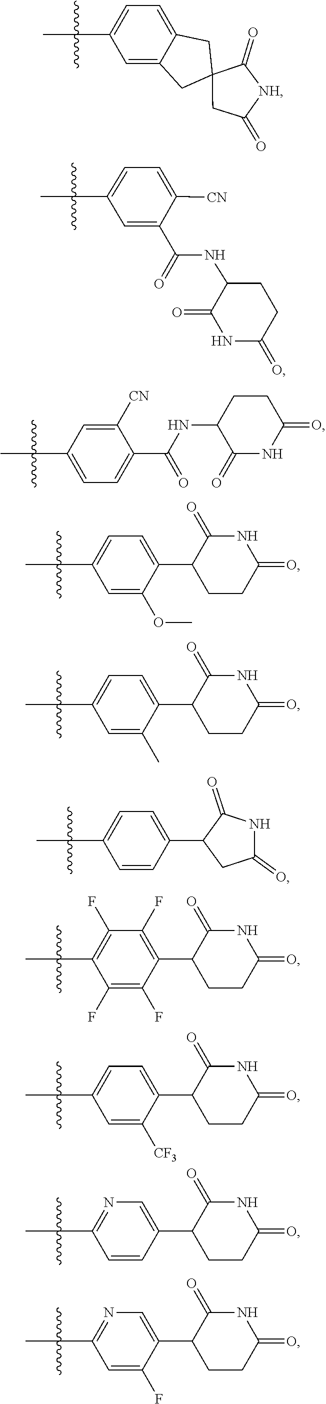

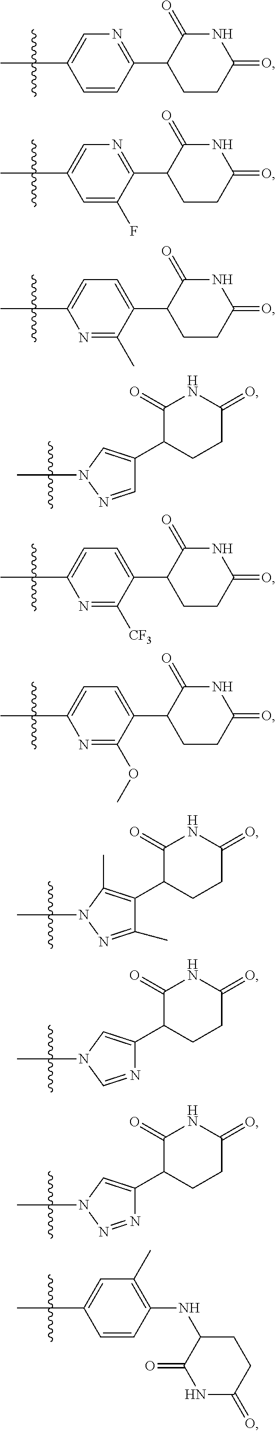

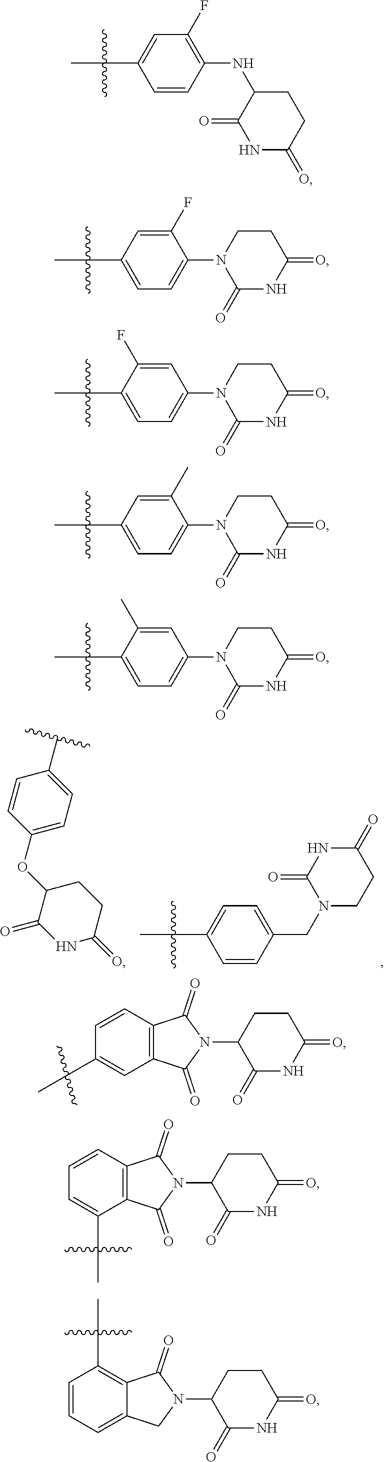

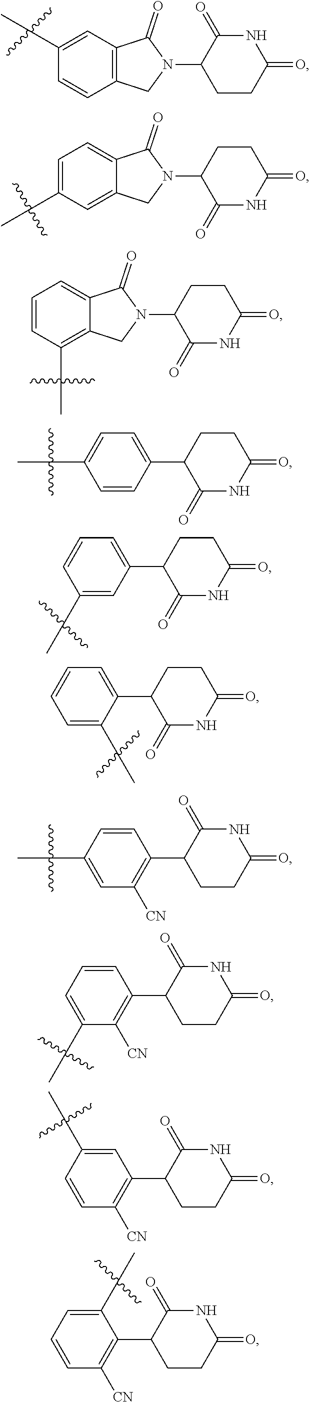

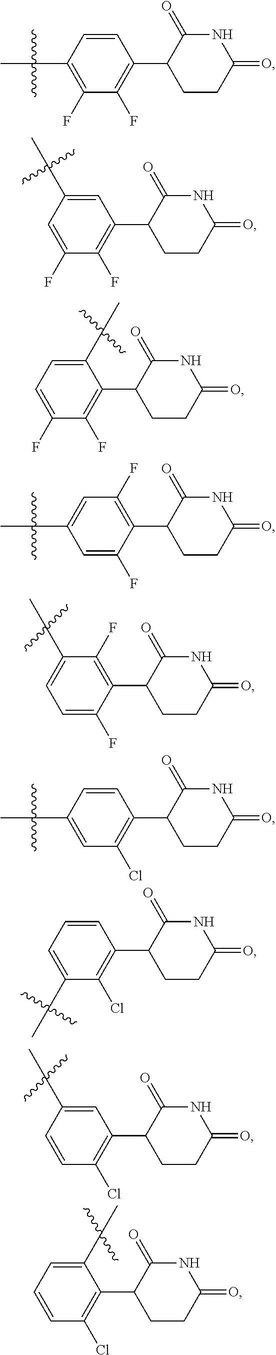

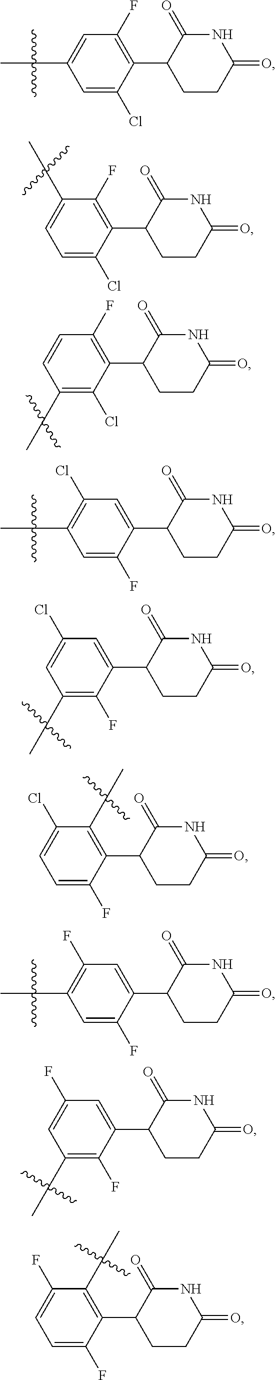

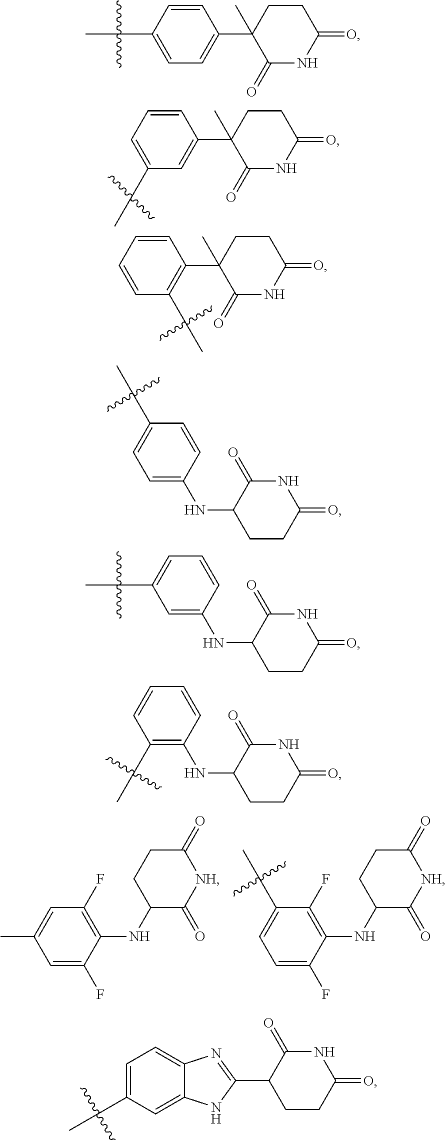

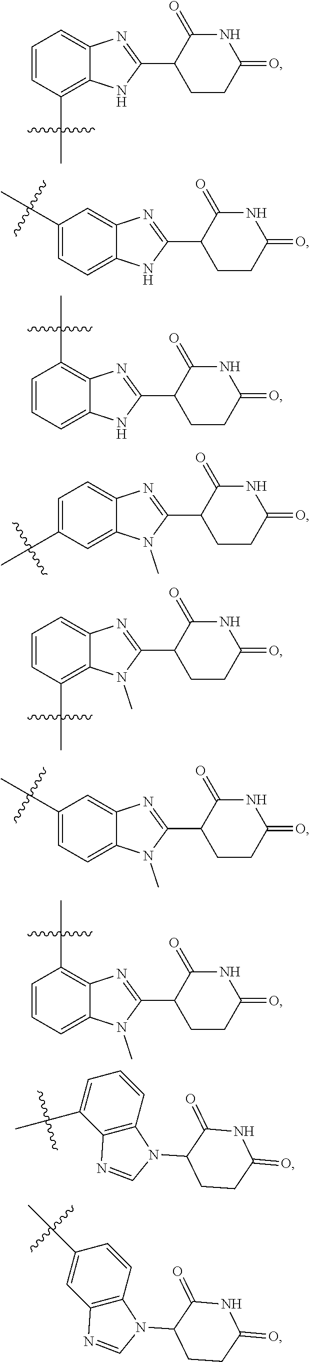

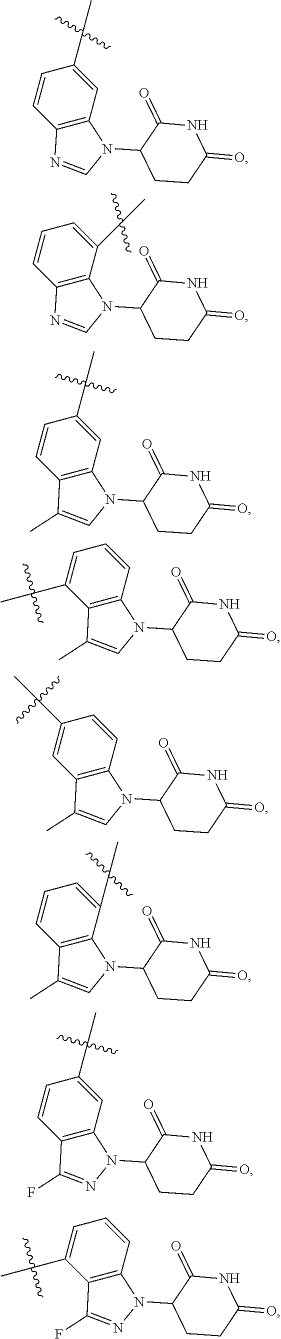

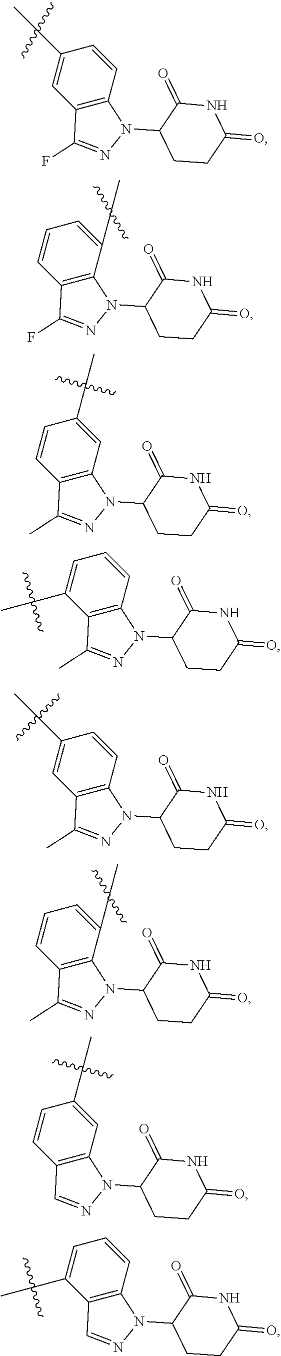

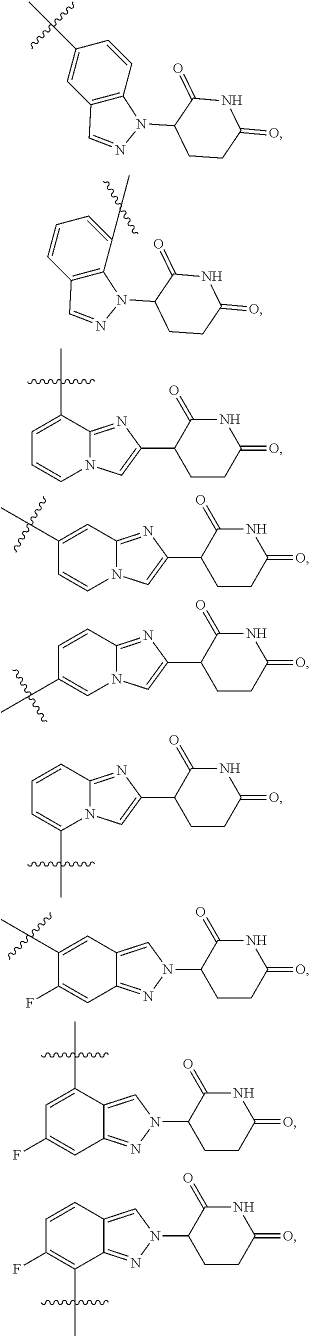

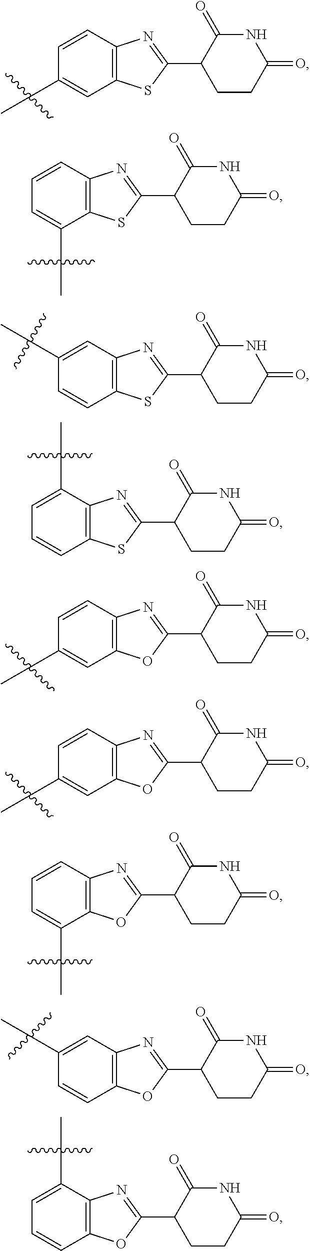

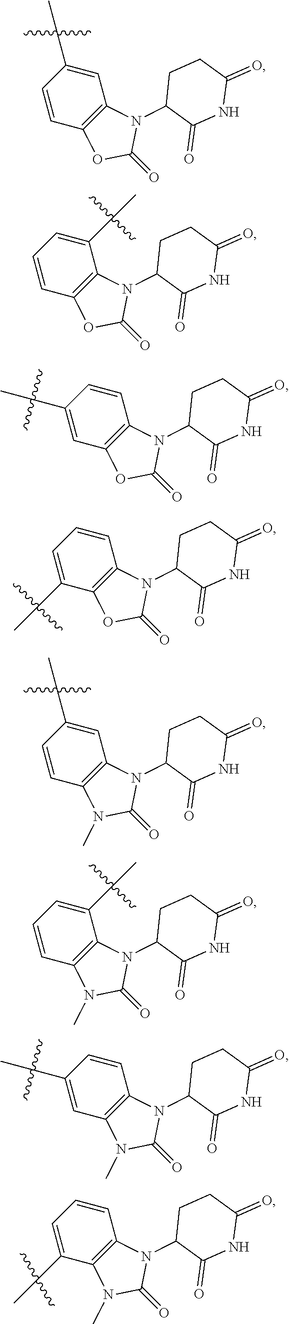

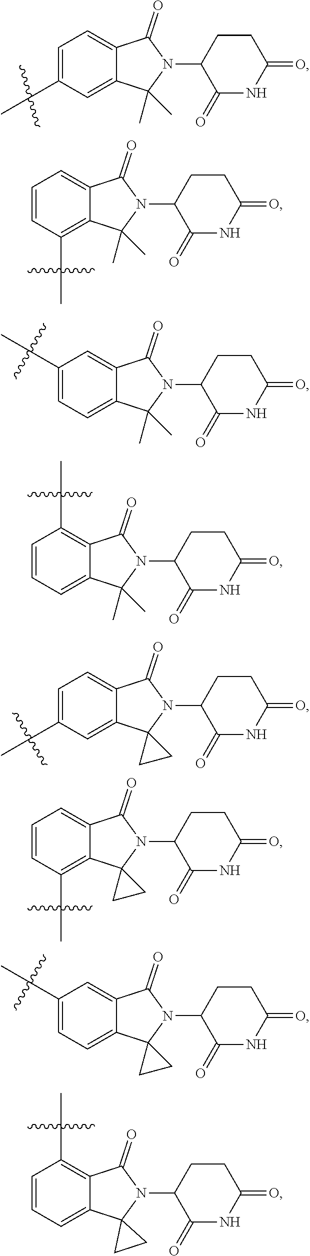

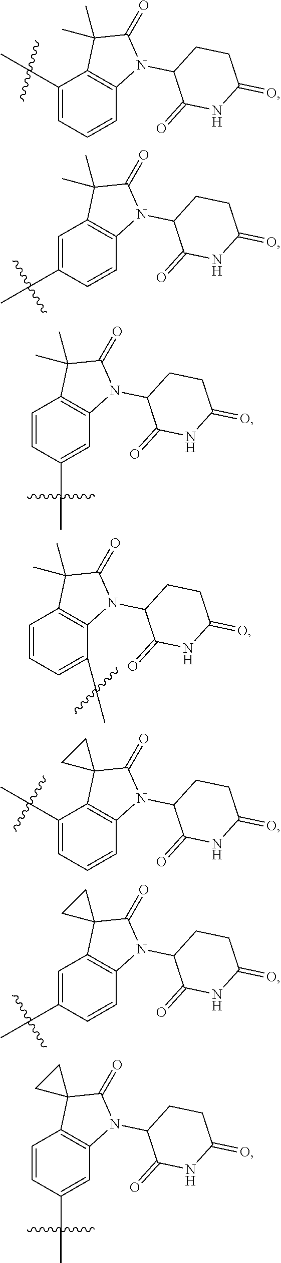

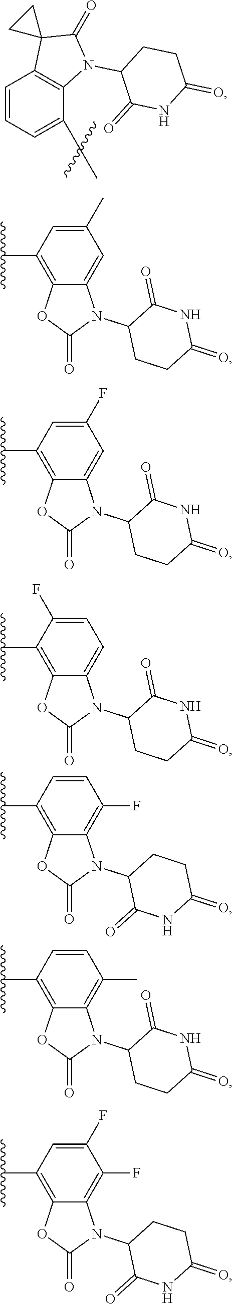

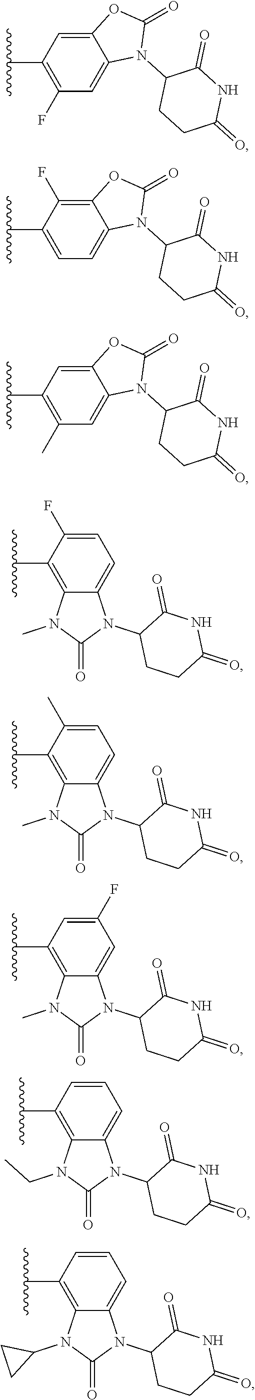

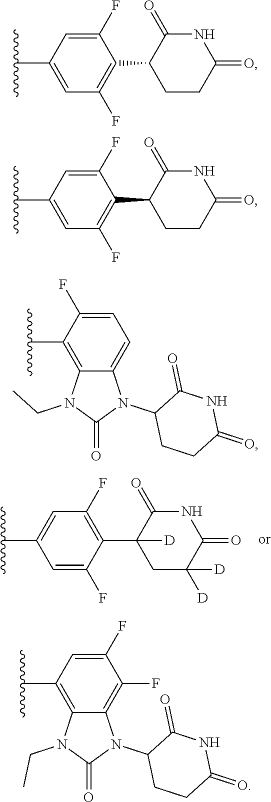

Aspect 47. The compound of any one of Aspects 1-46, wherein Z¹, Z², Z³ and Z⁴ are each independently —CR^{z};

R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one R^{Zc};

R^{Za} and R^{Zb} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};

R^{Zc} and R^{Zb} are each independently —F, —Cl, —Br, —I, hydroxy, methyl, ethyl, propyl, butyl, pentyl, hexyl, heptyl, octyl, —C₁-C₈alkoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 48. The compound of any one of Aspects 1-47, wherein R^{z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃, —CH(OH)CH₃,

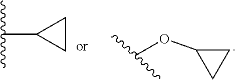

Aspect 49. The compound of any one of Aspects 1-48 selected from the compound of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 465, 466, 467, 468, 469, 470, 471, 472, 473, 474, 476, 477, 478, 479, 480, 481, 482, 483, 484, 485, 486, 487, 488, 489, 490, 491, 492, 493, 496, 497, 498, 499, 500, 501, 502, 503, 504, 505, 506, 507, 508, 509, 510, 511, 512, 513, 514, 515, 516, 517, 518, 519, 520, 521, 522, 523, 525, 526, 527, 528, 529, 530, 531, 532, 533, 534, 535, 536, 537, 538, 539, 541, 542, 543, 544, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 581, 582, 583, 584, 585, 586, 587, 588, 589, 590, 591, 592, 593, 594, 595, 596, 597, 598, 599, 600, 601, 602, 603, 604, 605, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 639, 640, 641, 642, 643, 644, 645, 646, 647, 648, 649, 650, 651, 652, 653, 654, 655, 656, 657, 658, 659, 660, 661, 662, 663, 665, 666, 667, 668, 669, 670, 671, 672, 673, 674, 675, 676, 677, 678, 679, 680, 681, 682, 683, 684, 685, 686, 687, 689, 690, 691, 692, 693, 694, 695, 696, 697, 698, 699, 700, 701, 703, 704, 705, 706, 707, 708, 709, 710, 711, 712, 713, 714, 715, 716, 717, 718, 719, 720, 721, 723, 724, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755 or 756.

One another aspect, the compound of any one of Aspects 1-48 selected from

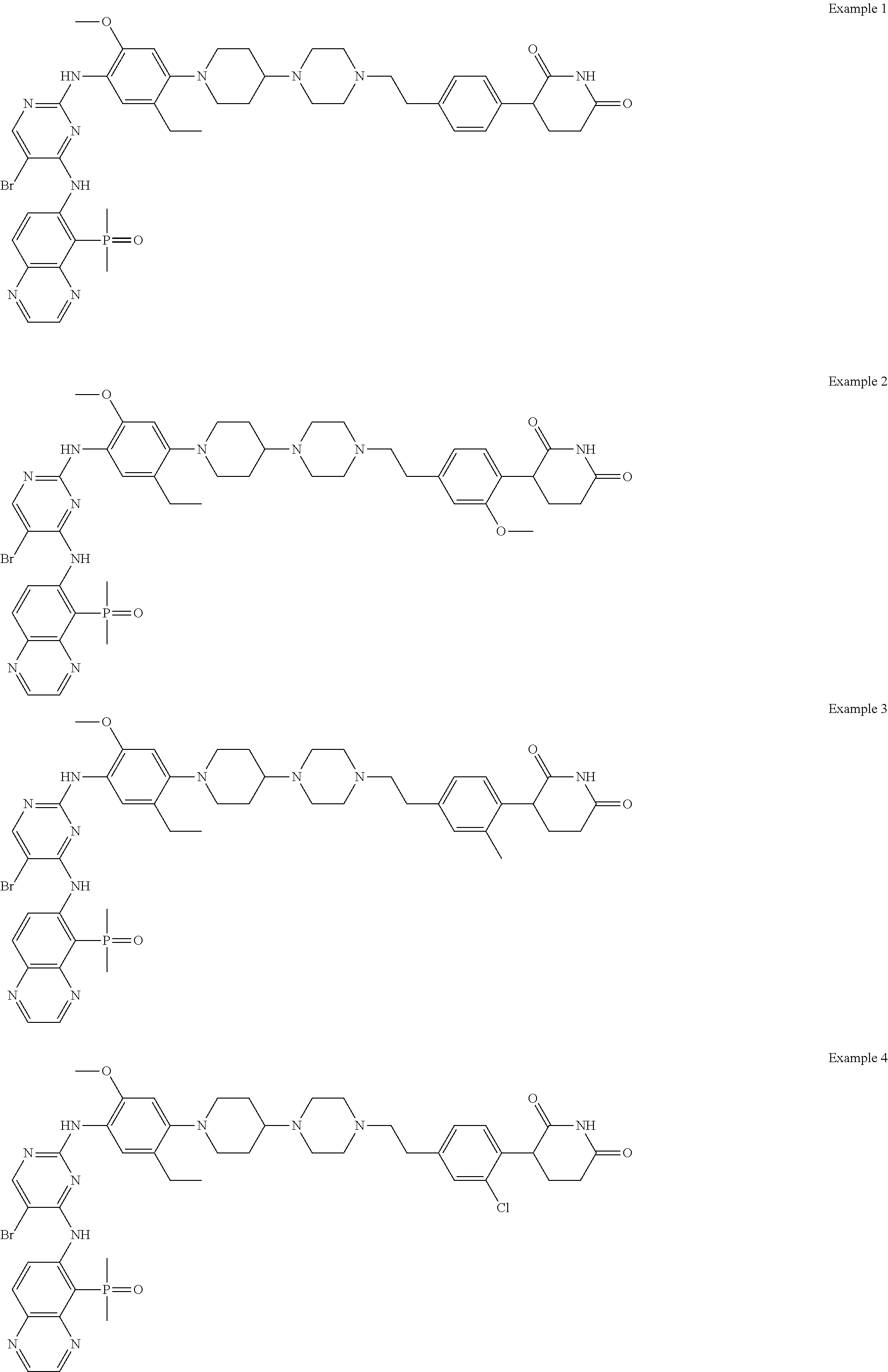

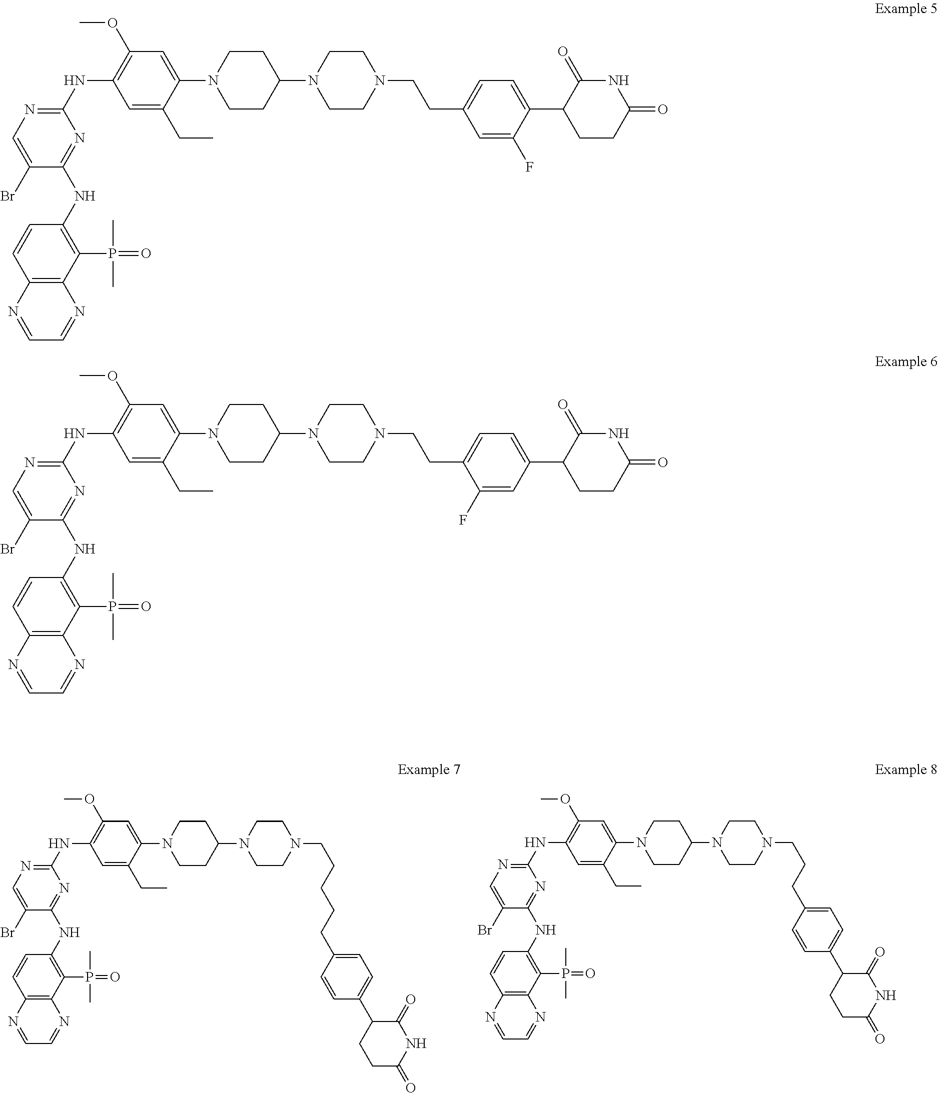 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(F)c3)CC2)CC1 ; CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)cc3F)CC2)CC1 ; CCc1cc(Nc2ncc(Br)c(Nc3ccc4nccnc4c3P(C)(C)=O)n2)c(OC)cc1N1CCC(N2CCN(CCCCCc3ccc(C4CCC(=O)NC4=O)cc3)CC2)CC1 ; CCc1cc(Nc2ncc(Br)c(Nc3ccc4nccnc4c3P(C)(C)=O)n2)c(OC)cc1N1CCC(N2CCN(CCCc3ccc(C4CCC(=O)NC4=O)cc3)CC2)CC1]

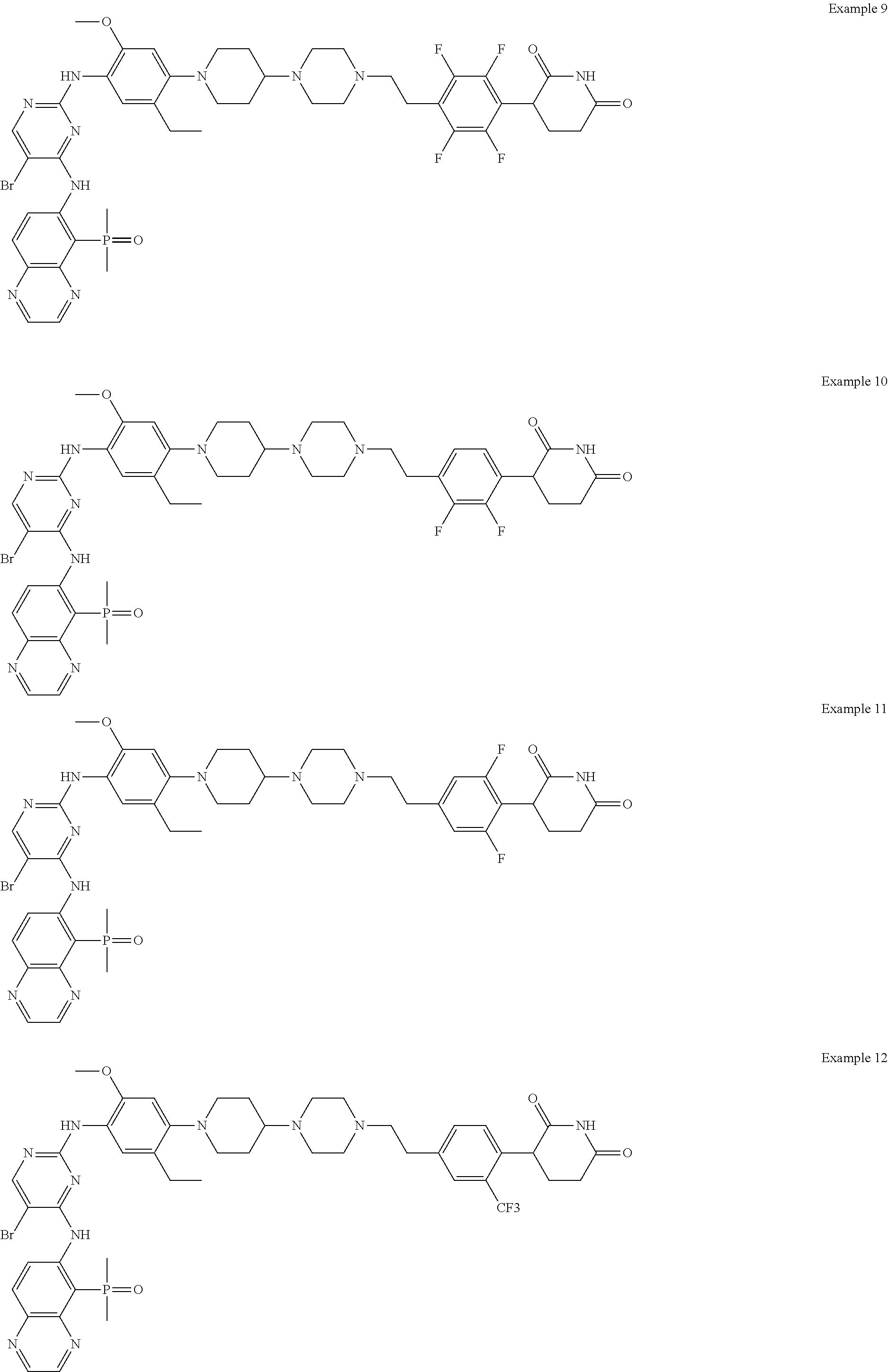

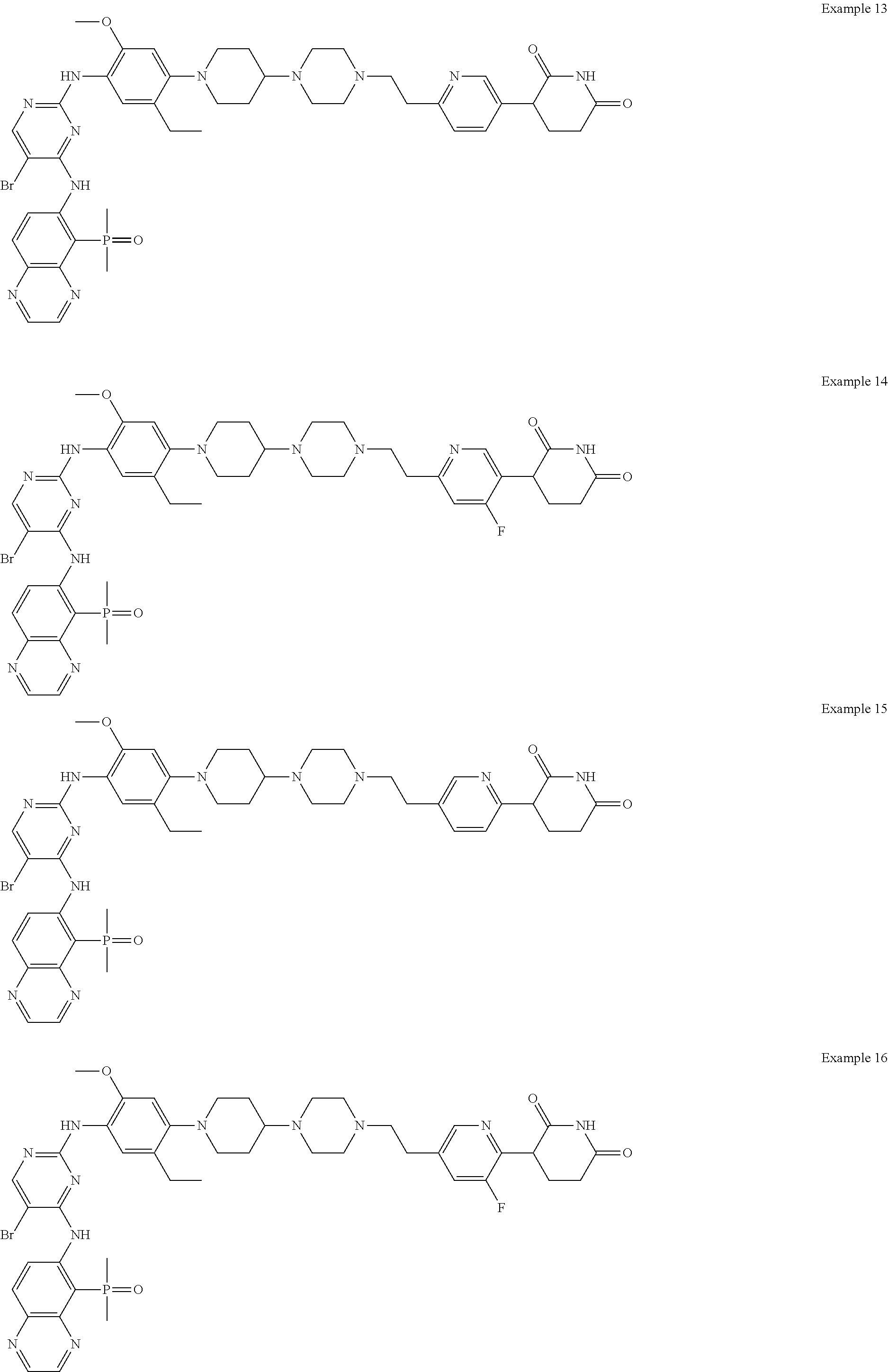

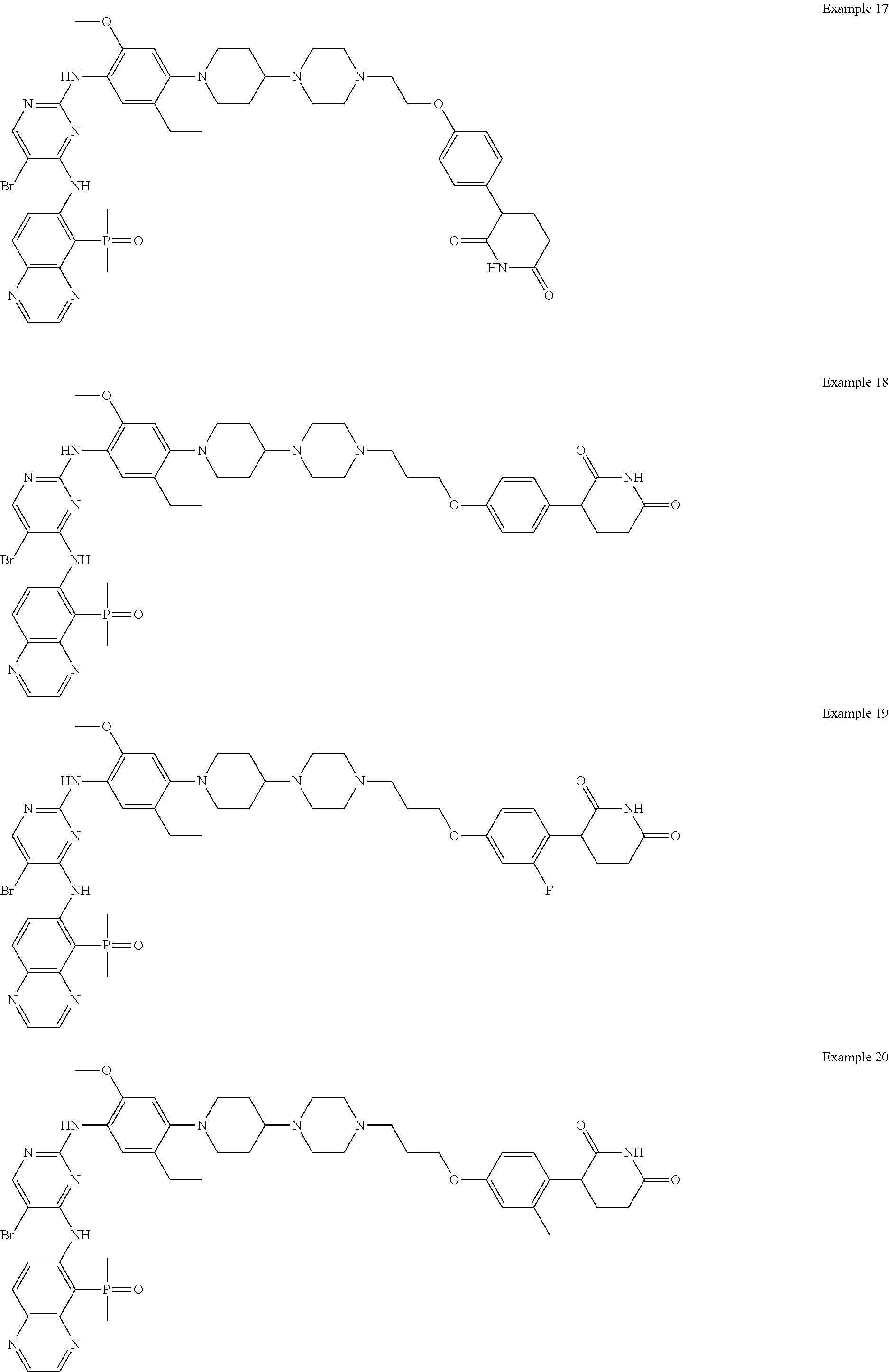

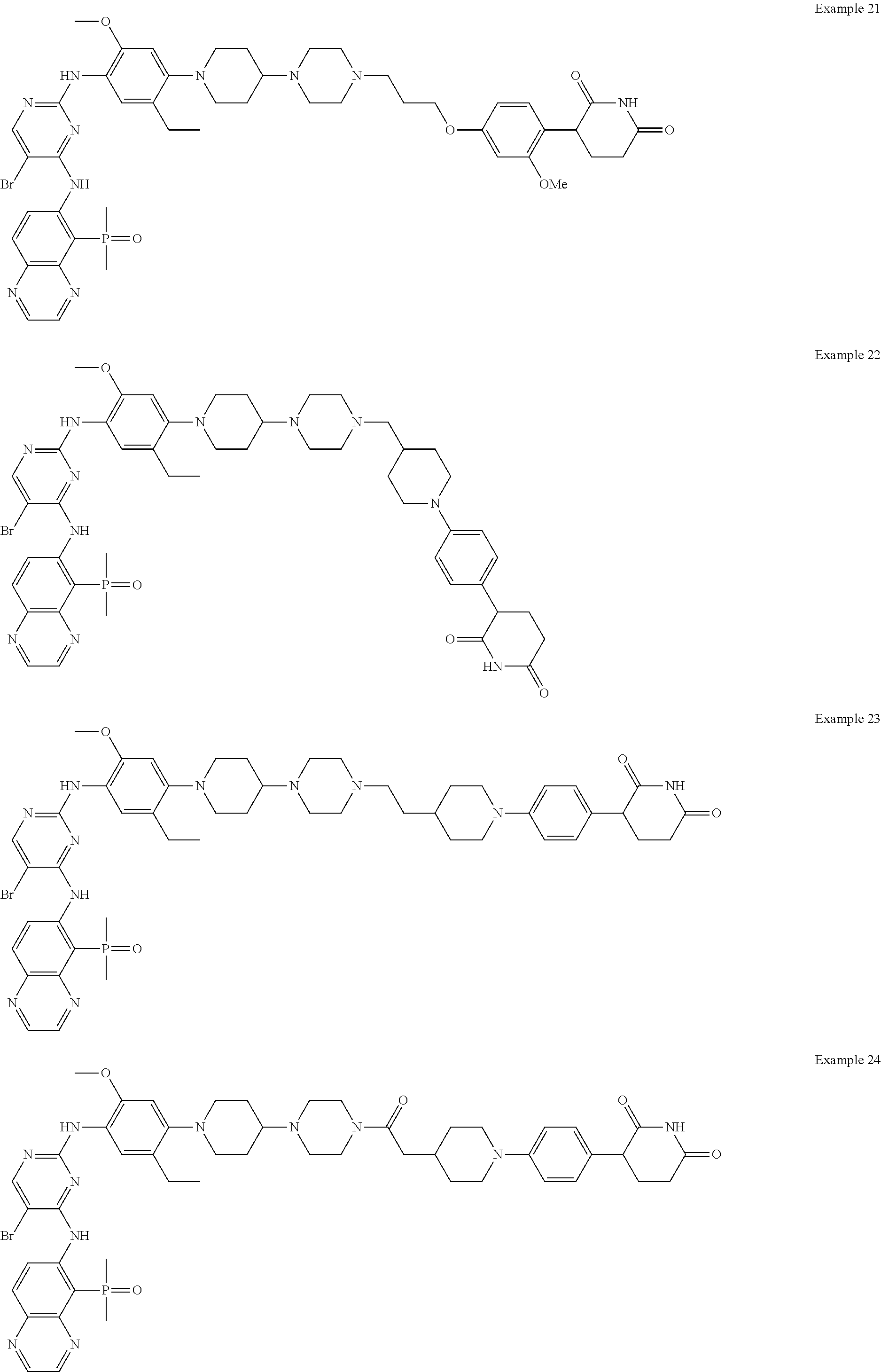 [SMILES: CCc1cc(Nc2ncc(Br)c(Nc3ccc4nccnc4c3P(C)(C)=O)n2)c(OC)cc1N1CCC(N2CCN(CCCOc3ccc(C4CCC(=O)NC4=O)c(OC)c3)CC2)CC1 ; CCc1cc(Nc2ncc(Br)c(Nc3ccc4nccnc4c3P(C)(C)=O)n2)c(OC)cc1N1CCC(N2CCN(CC3CCN(c4ccc(C5CCC(=O)NC5=O)cc4)CC3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4ccc(C5CCC(=O)NC5=O)cc4)CC3)CC2)CC1 ; CCc1cc(Nc2ncc(Br)c(Nc3ccc4nccnc4c3P(C)(C)=O)n2)c(OC)cc1N1CCC(N2CCN(C(=O)CC3CCN(c4ccc(C5CCC(=O)NC5=O)cc4)CC3)CC2)CC1]

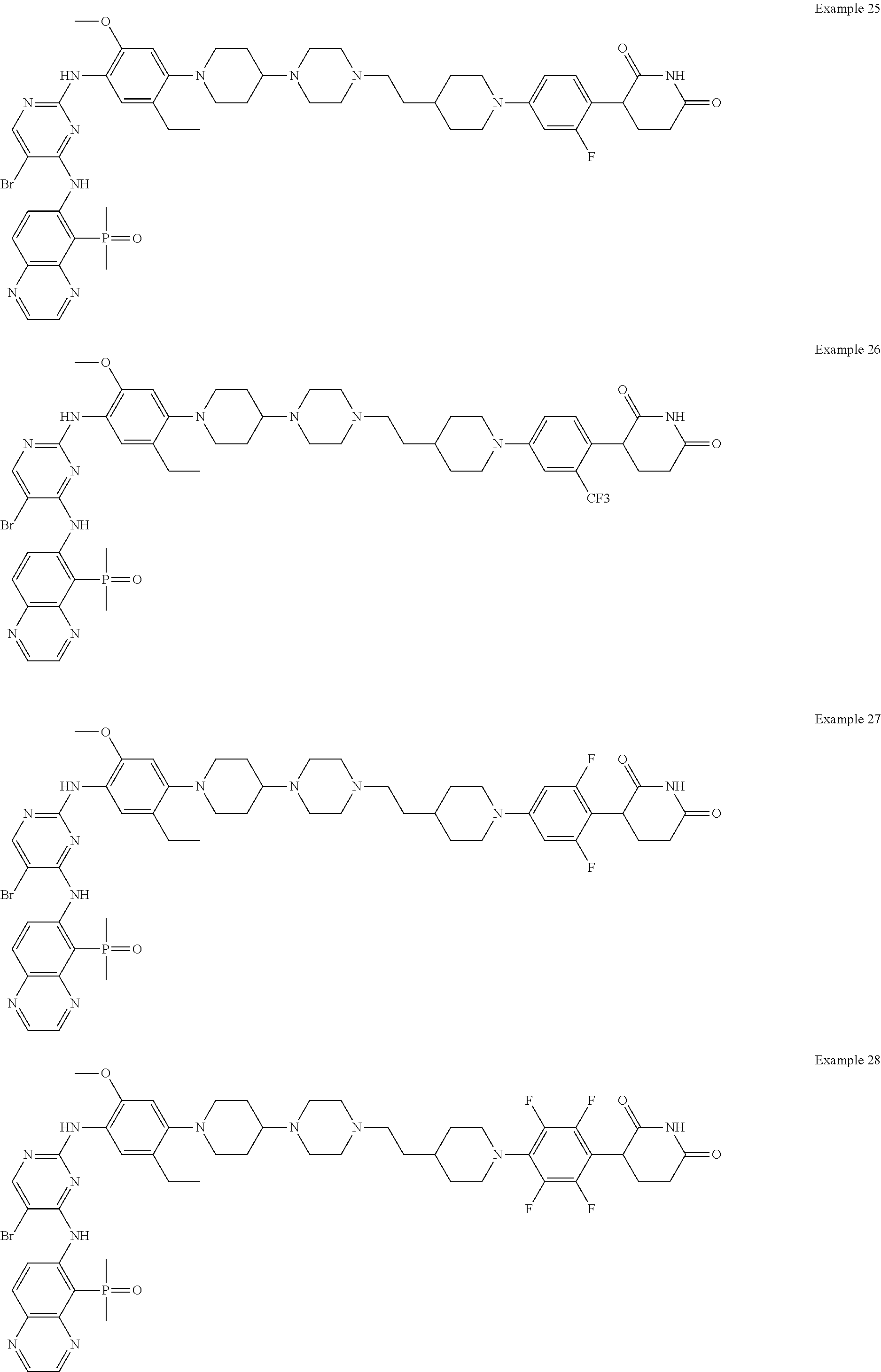 [SMILES: CCc1cc(Nc2ncc(Br)c(Nc3ccc4nccnc4c3P(C)(C)=O)n2)c(OC)cc1N1CCC(N2CCN(CCC3CCN(c4ccc(C5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4ccc(C5CCC(=O)NC5=O)c(C(F)(F)F)c4)CC3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4c(F)c(F)c(C5CCC(=O)NC5=O)c(F)c4F)CC3)CC2)CC1]

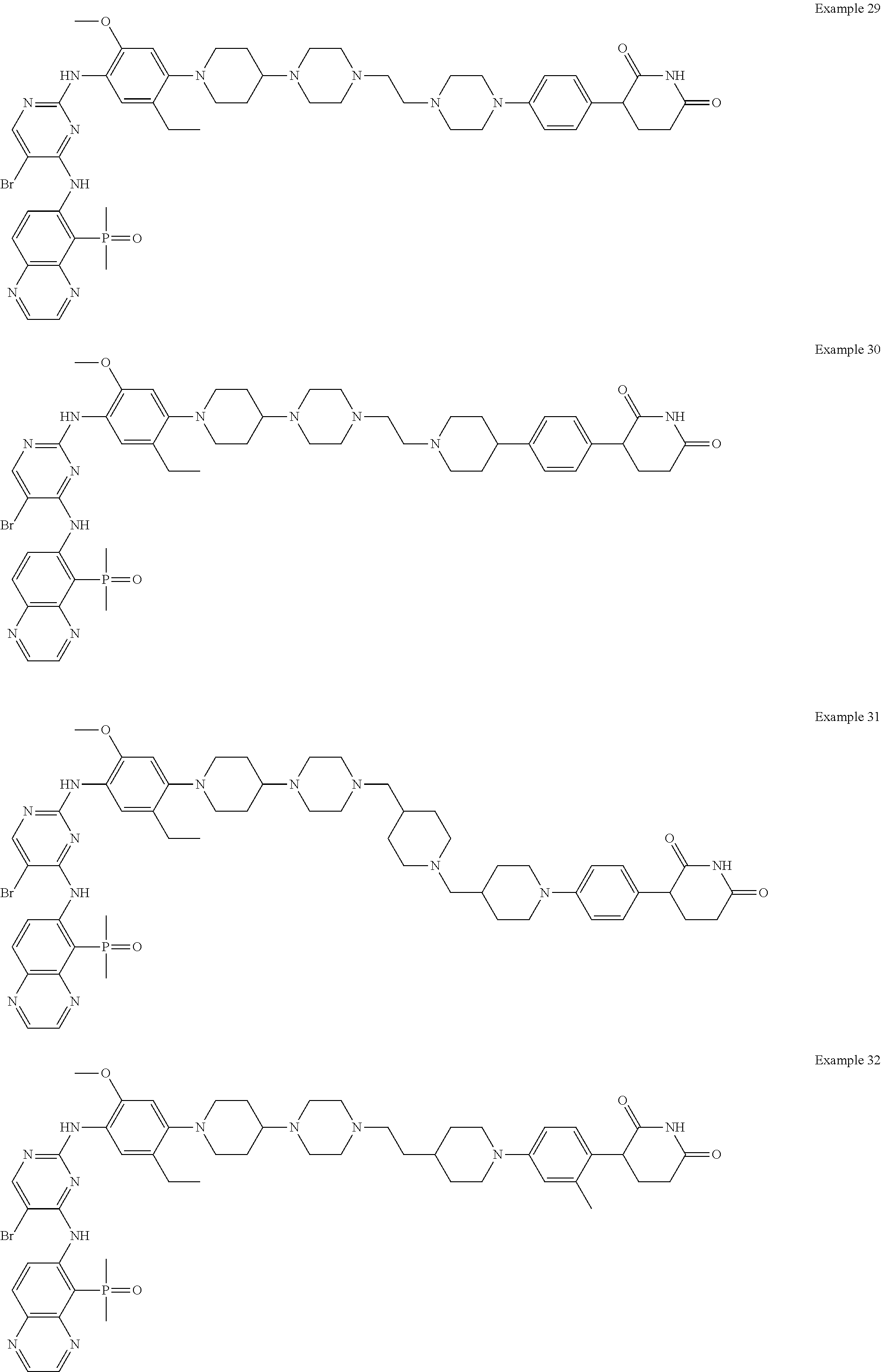 [SMILES: CCc1cc(Nc2ncc(Br)c(Nc3ccc4nccnc4c3P(C)(C)=O)n2)c(OC)cc1N1CCC(N2CCN(CCN3CCN(c4ccc(C5CCC(=O)NC5=O)cc4)CC3)CC2)CC1 ; CCc1cc(Nc2ncc(Br)c(Nc3ccc4nccnc4c3P(C)(C)=O)n2)c(OC)cc1N1CCC(N2CCN(CCN3CCC(c4ccc(C5CCC(=O)NC5=O)cc4)CC3)CC2)CC1 ; CCc1cc(Nc2ncc(Br)c(Nc3ccc4nccnc4c3P(C)(C)=O)n2)c(OC)cc1N1CCC(N2CCN(CC3CCN(CC4CCN(c5ccc(C6CCC(=O)NC6=O)cc5)CC4)CC3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4ccc(C5CCC(=O)NC5=O)c(C)c4)CC3)CC2)CC1]

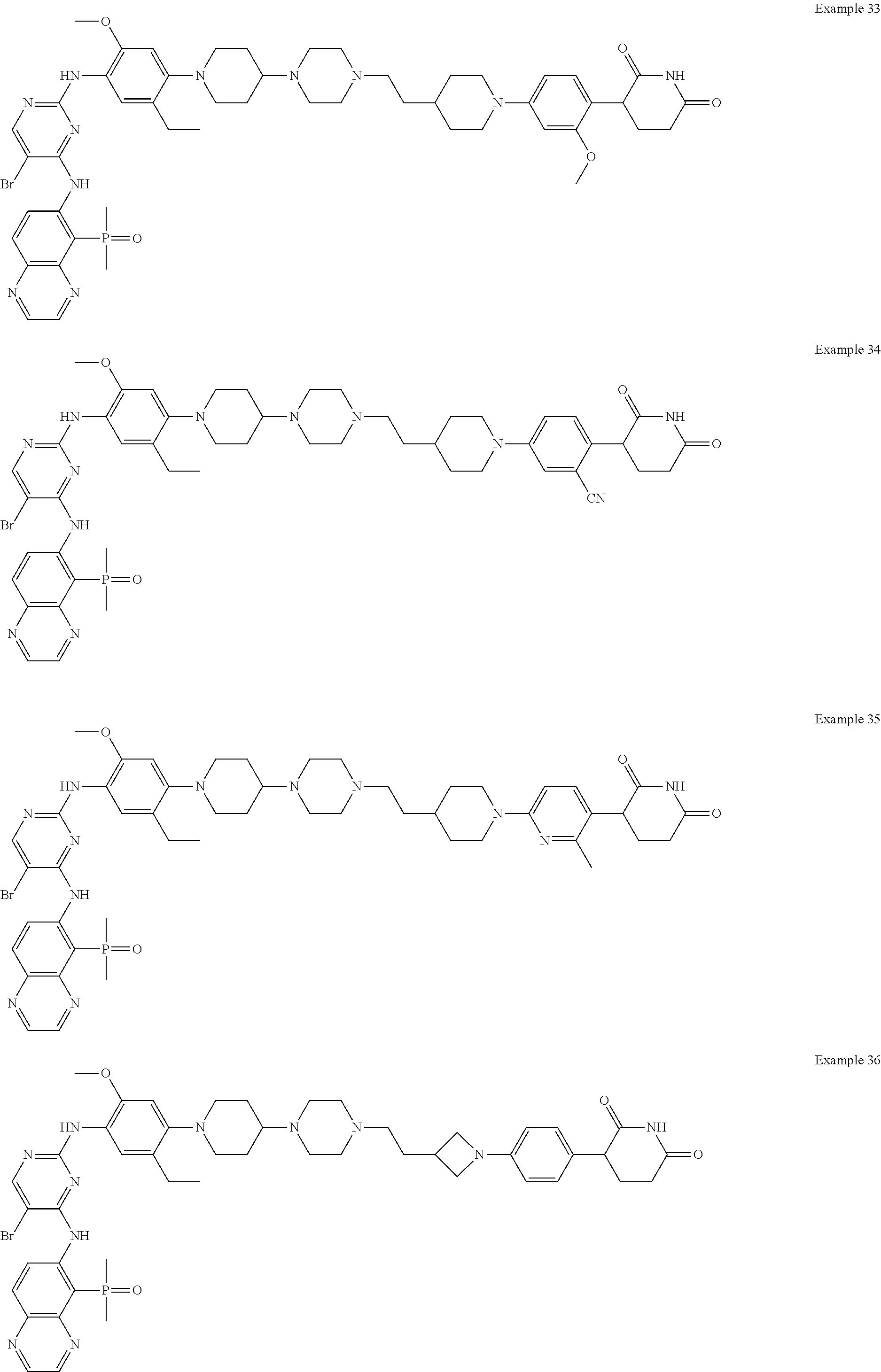 [SMILES: CCc1cc(Nc2ncc(Br)c(Nc3ccc4nccnc4c3P(C)(C)=O)n2)c(OC)cc1N1CCC(N2CCN(CCC3CCN(c4ccc(C5CCC(=O)NC5=O)c(OC)c4)CC3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4ccc(C5CCC(=O)NC5=O)c(C#N)c4)CC3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4ccc(C5CCC(=O)NC5=O)c(C)n4)CC3)CC2)CC1 ; CCc1cc(Nc2ncc(Br)c(Nc3ccc4nccnc4c3P(C)(C)=O)n2)c(OC)cc1N1CCC(N2CCN(CCC3CN(c4ccc(C5CCC(=O)NC5=O)cc4)C3)CC2)CC1]

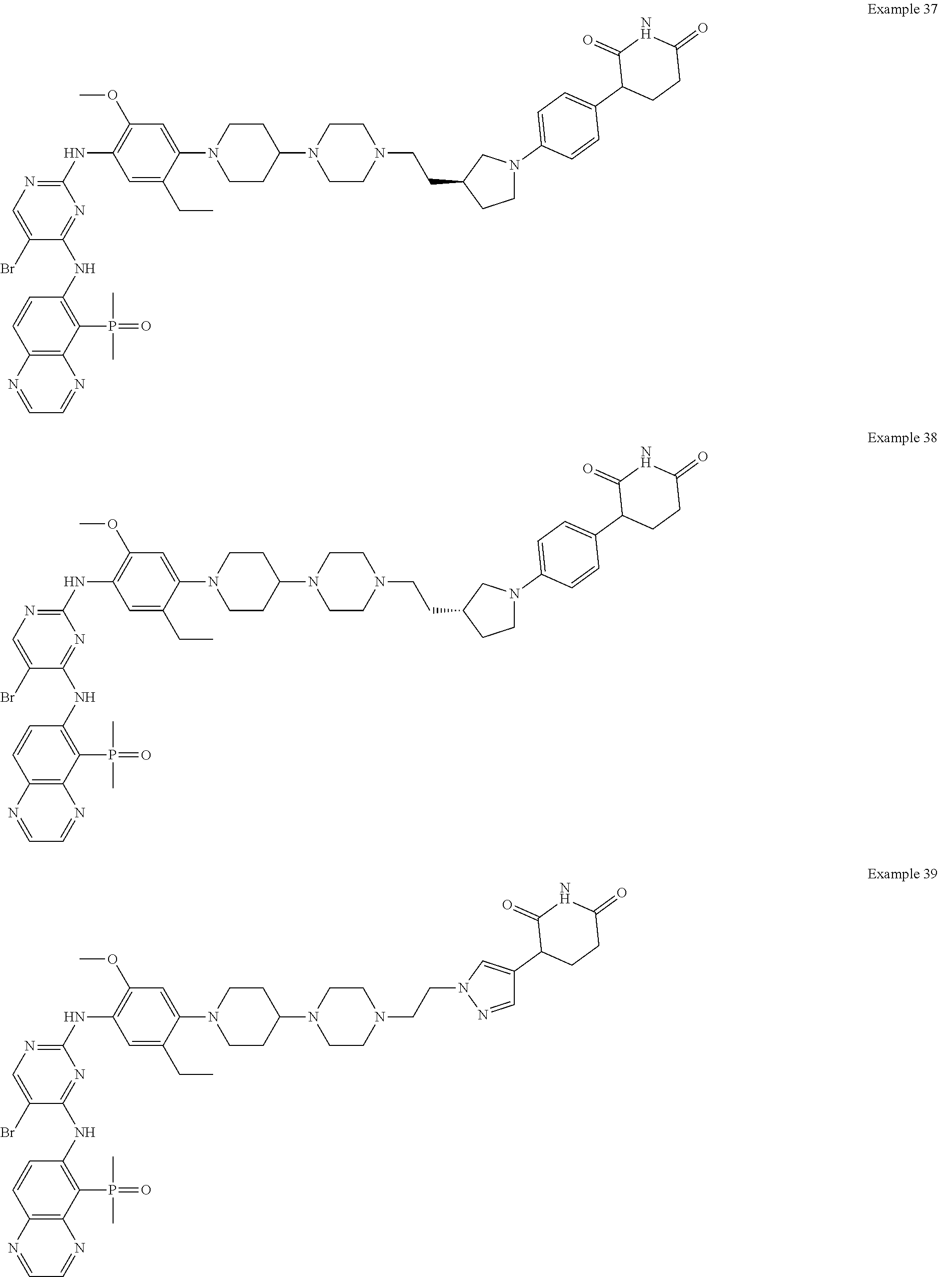 [SMILES: CCc1cc(Nc2ncc(Br)c(Nc3ccc4nccnc4c3P(C)(C)=O)n2)c(OC)cc1N1CCC(N2CCN(CC[C@@H]3CCN(c4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(CC[C@H]3CCN(c4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(CCn3cc(C4CCC(=O)NC4=O)cn3)CC2)CC1]

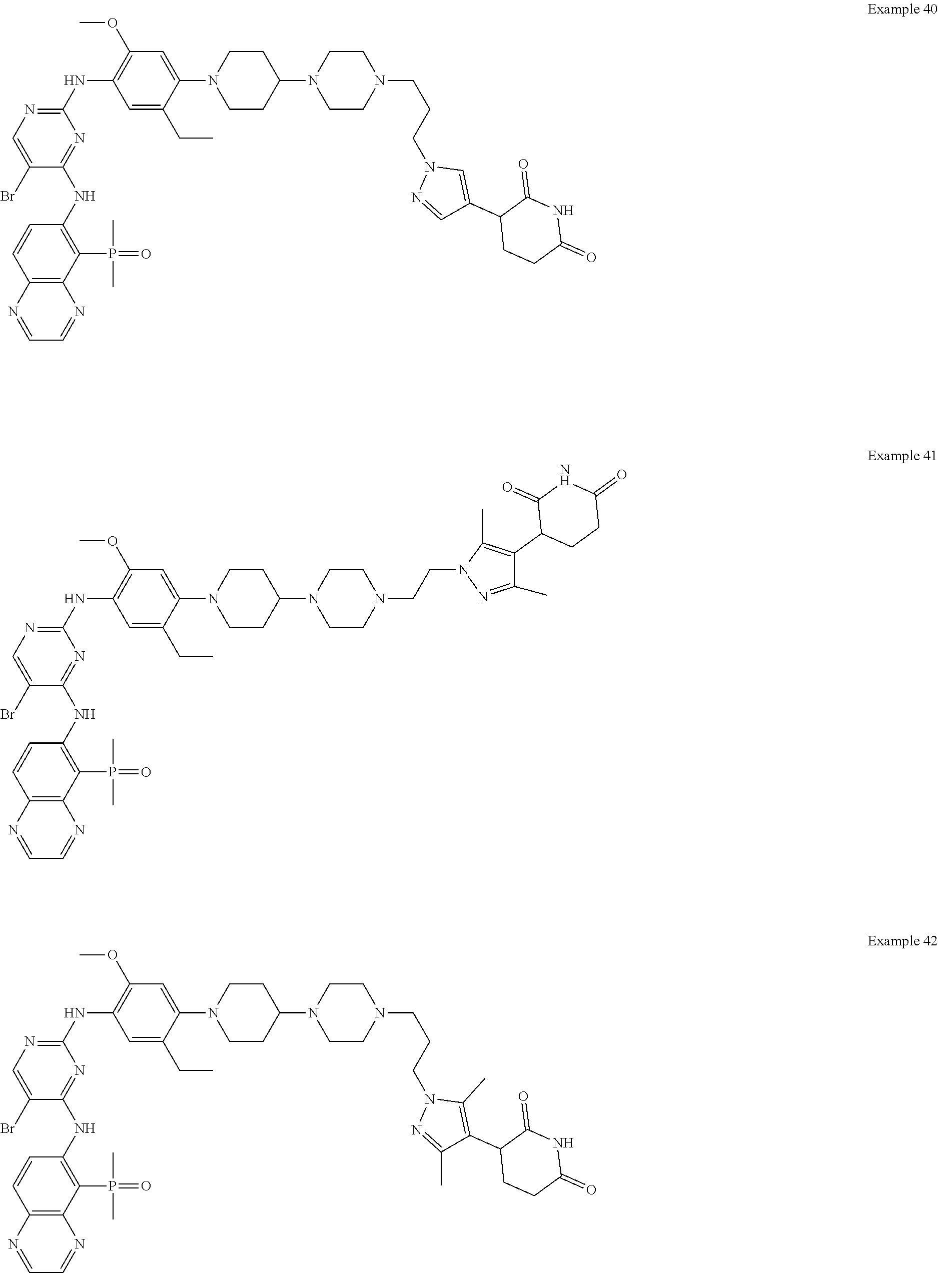 [SMILES: CCc1cc(Nc2ncc(Br)c(Nc3ccc4nccnc4c3P(C)(C)=O)n2)c(OC)cc1N1CCC(N2CCN(CCCn3cc(C4CCC(=O)NC4=O)cn3)CC2)CC1 ; CCc1cc(Nc2ncc(Br)c(Nc3ccc4nccnc4c3P(C)(C)=O)n2)c(OC)cc1N1CCC(N2CCN(CCn3nc(C)c(C4CCC(=O)NC4=O)c3C)CC2)CC1 ; CCc1cc(Nc2ncc(Br)c(Nc3ccc4nccnc4c3P(C)(C)=O)n2)c(OC)cc1N1CCC(N2CCN(CCCn3nc(C)c(C4CCC(=O)NC4=O)c3C)CC2)CC1]

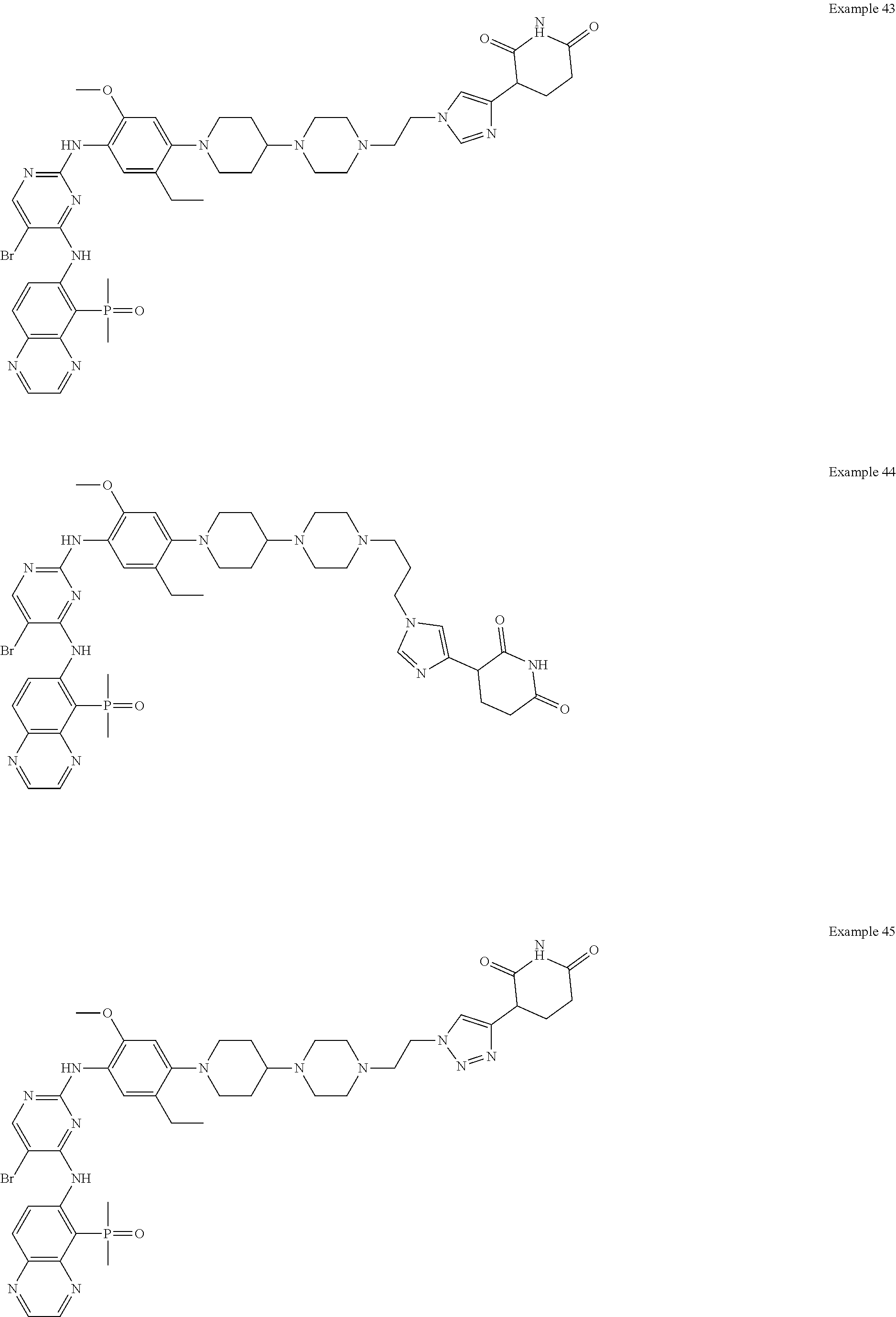 [SMILES: CCc1cc(Nc2ncc(Br)c(Nc3ccc4nccnc4c3P(C)(C)=O)n2)c(OC)cc1N1CCC(N2CCN(CCn3cnc(C4CCC(=O)NC4=O)c3)CC2)CC1 ; CCc1cc(Nc2ncc(Br)c(Nc3ccc4nccnc4c3P(C)(C)=O)n2)c(OC)cc1N1CCC(N2CCN(CCCn3cnc(C4CCC(=O)NC4=O)c3)CC2)CC1 ; CCc1cc(Nc2ncc(Br)c(Nc3ccc4nccnc4c3P(C)(C)=O)n2)c(OC)cc1N1CCC(N2CCN(CCn3cc(C4CCC(=O)NC4=O)nn3)CC2)CC1]

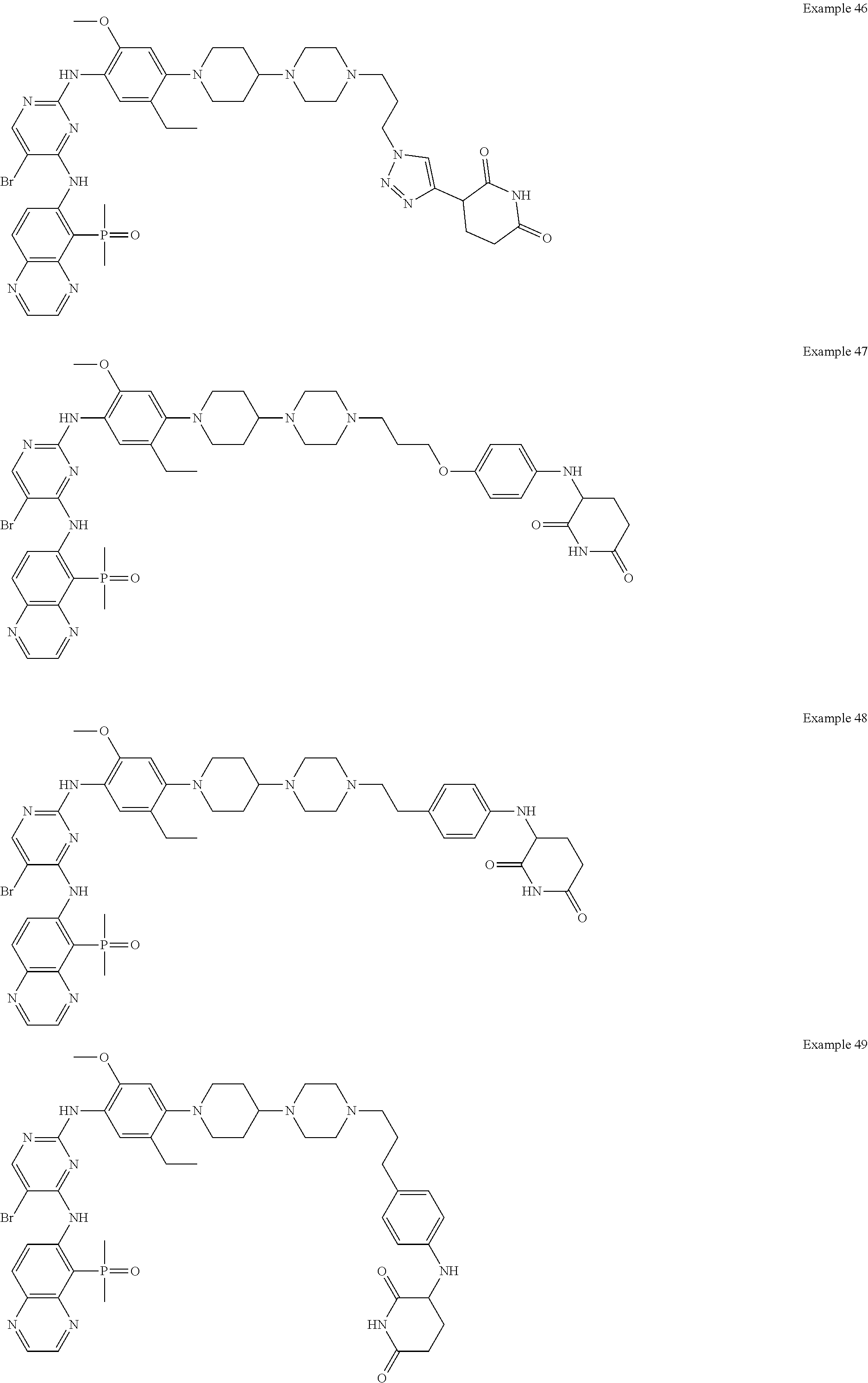

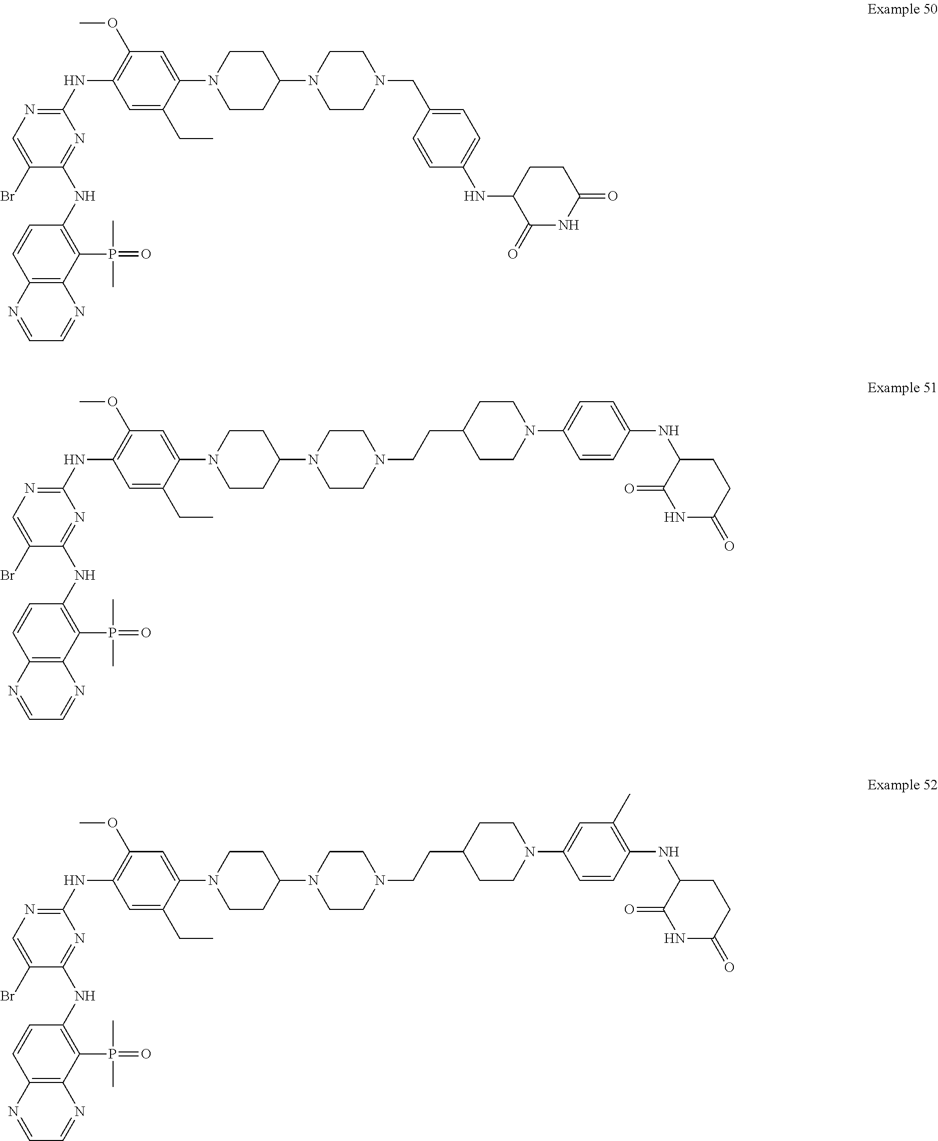 [SMILES: CCc1cc(Nc2ncc(Br)c(Nc3ccc4nccnc4c3P(C)(C)=O)n2)c(OC)cc1N1CCC(N2CCN(Cc3ccc(NC4CCC(=O)NC4=O)cc3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4ccc(NC5CCC(=O)NC5=O)cc4)CC3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4ccc(NC5CCC(=O)NC5=O)c(C)c4)CC3)CC2)CC1]

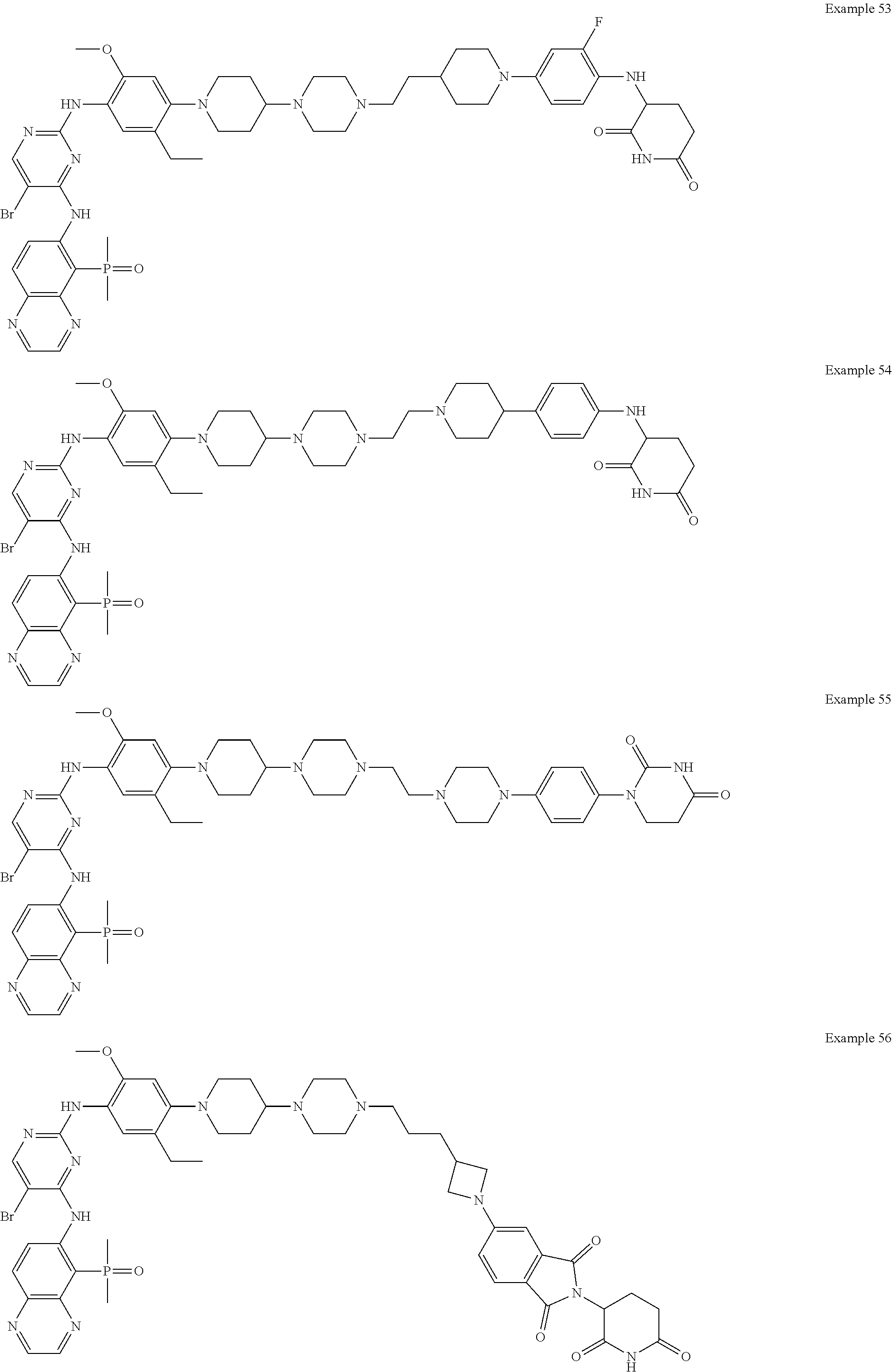

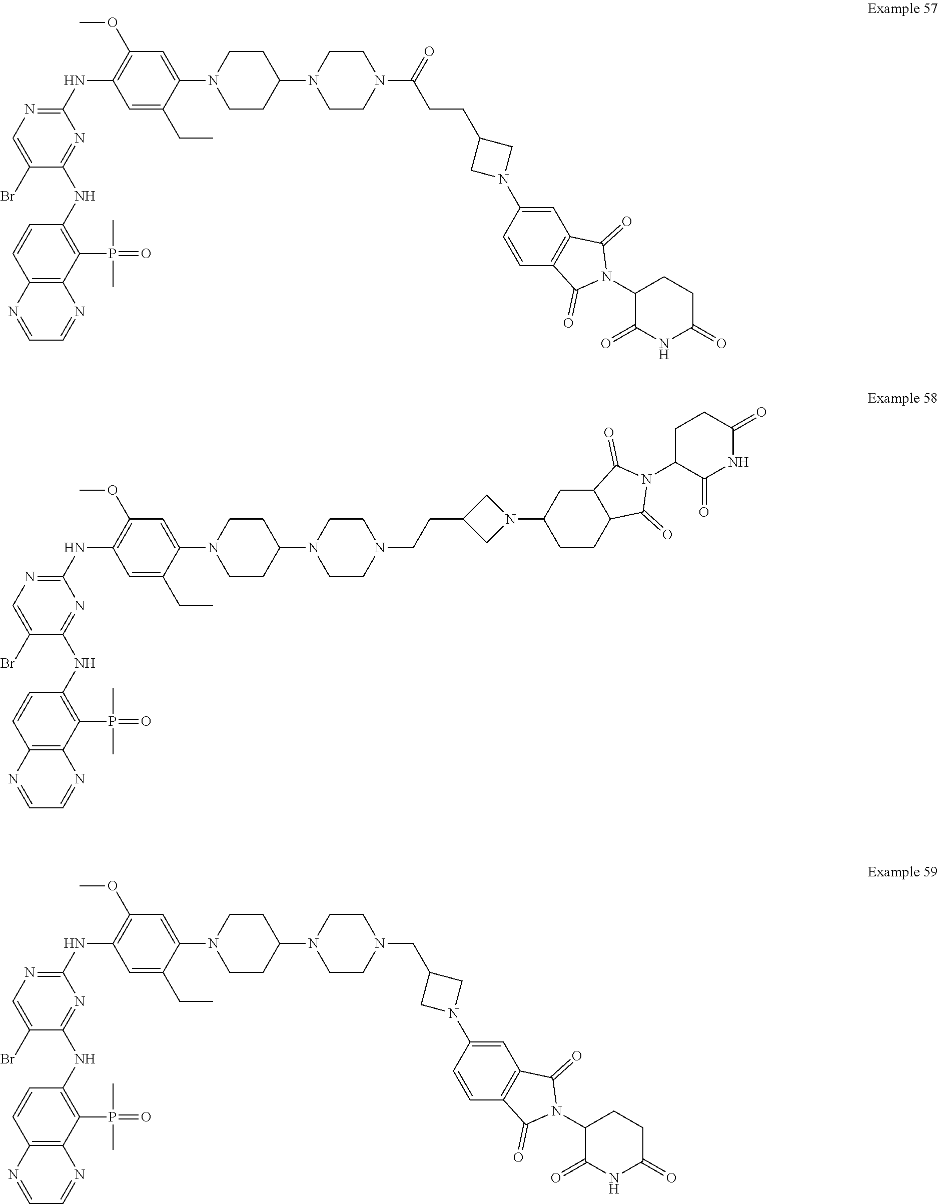 [SMILES: CCc1cc(Nc2ncc(Br)c(Nc3ccc4nccnc4c3P(C)(C)=O)n2)c(OC)cc1N1CCC(N2CCN(C(=O)CCC3CN(c4ccc5c(c4)C(=O)N(C4CCC(=O)NC4=O)C5=O)C3)CC2)CC1 ; CCc1cc(Nc2ncc(Br)c(Nc3ccc4nccnc4c3P(C)(C)=O)n2)c(OC)cc1N1CCC(N2CCN(CCC3CN(C4CCC5C(=O)N(C6CCC(=O)NC6=O)C(=O)C5C4)C3)CC2)CC1 ; CCc1cc(Nc2ncc(Br)c(Nc3ccc4nccnc4c3P(C)(C)=O)n2)c(OC)cc1N1CCC(N2CCN(CC3CN(c4ccc5c(c4)C(=O)N(C4CCC(=O)NC4=O)C5=O)C3)CC2)CC1]

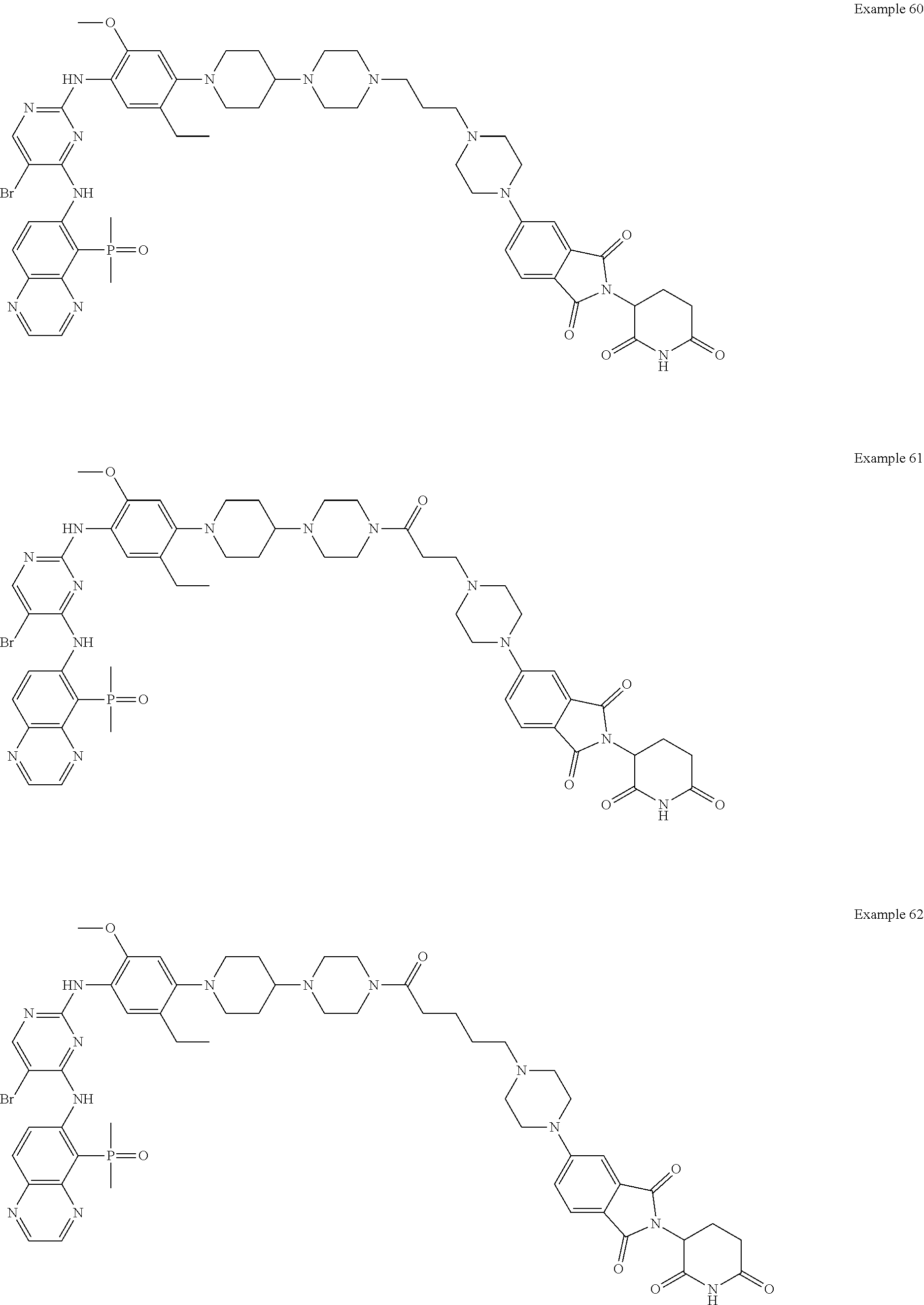 [SMILES: CCc1cc(Nc2ncc(Br)c(Nc3ccc4nccnc4c3P(C)(C)=O)n2)c(OC)cc1N1CCC(N2CCN(CCCN3CCN(c4ccc5c(c4)C(=O)N(C4CCC(=O)NC4=O)C5=O)CC3)CC2)CC1 ; CCc1cc(Nc2ncc(Br)c(Nc3ccc4nccnc4c3P(C)(C)=O)n2)c(OC)cc1N1CCC(N2CCN(C(=O)CCN3CCN(c4ccc5c(c4)C(=O)N(C4CCC(=O)NC4=O)C5=O)CC3)CC2)CC1 ; CCc1cc(Nc2ncc(Br)c(Nc3ccc4nccnc4c3P(C)(C)=O)n2)c(OC)cc1N1CCC(N2CCN(C(=O)CCCCN3CCN(c4ccc5c(c4)C(=O)N(C4CCC(=O)NC4=O)C5=O)CC3)CC2)CC1]

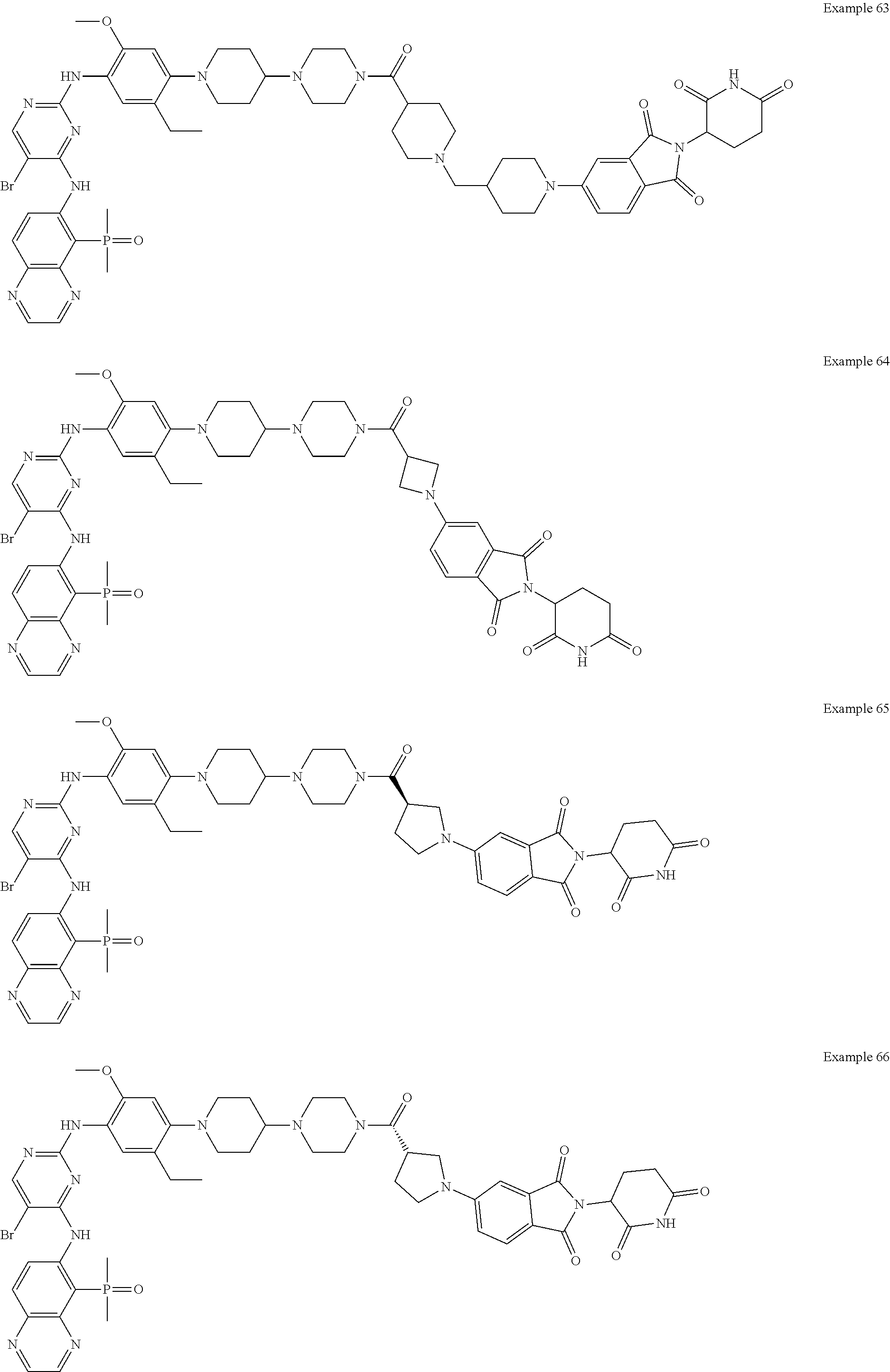

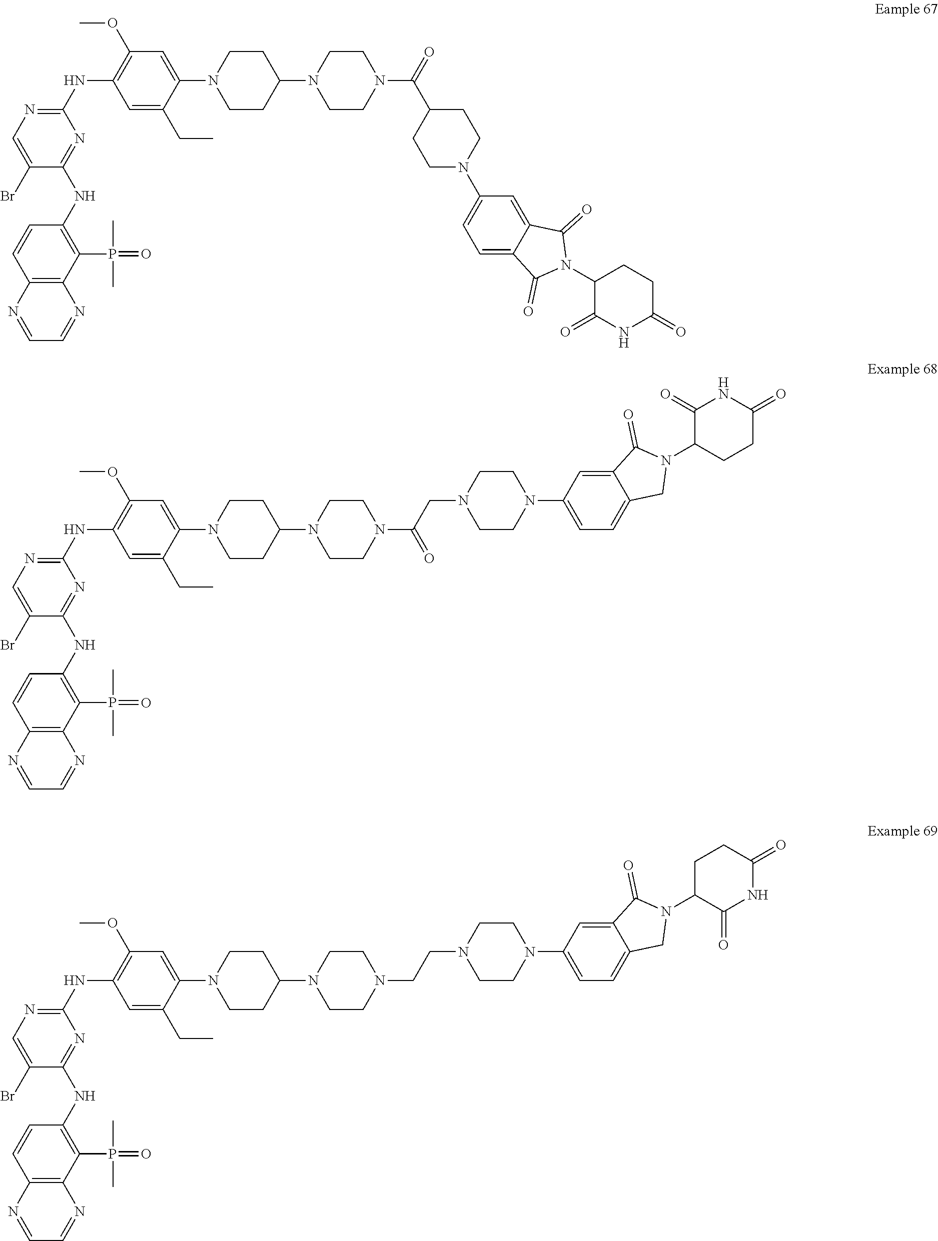 [SMILES: CCc1cc(Nc2ncc(Br)c(Nc3ccc4nccnc4c3P(C)(C)=O)n2)c(OC)cc1N1CCC(N2CCN(C(=O)C3CCN(c4ccc5c(c4)C(=O)N(C4CCC(=O)NC4=O)C5=O)CC3)CC2)CC1 ; CCc1cc(Nc2ncc(Br)c(Nc3ccc4nccnc4c3P(C)(C)=O)n2)c(OC)cc1N1CCC(N2CCN(C(=O)CN3CCN(c4ccc5c(c4)C(=O)N(C4CCC(=O)NC4=O)C5)CC3)CC2)CC1 ; CCc1cc(Nc2ncc(Br)c(Nc3ccc4nccnc4c3P(C)(C)=O)n2)c(OC)cc1N1CCC(N2CCN(CCN3CCN(c4ccc5c(c4)C(=O)N(C4CCC(=O)NC4=O)C5)CC3)CC2)CC1]

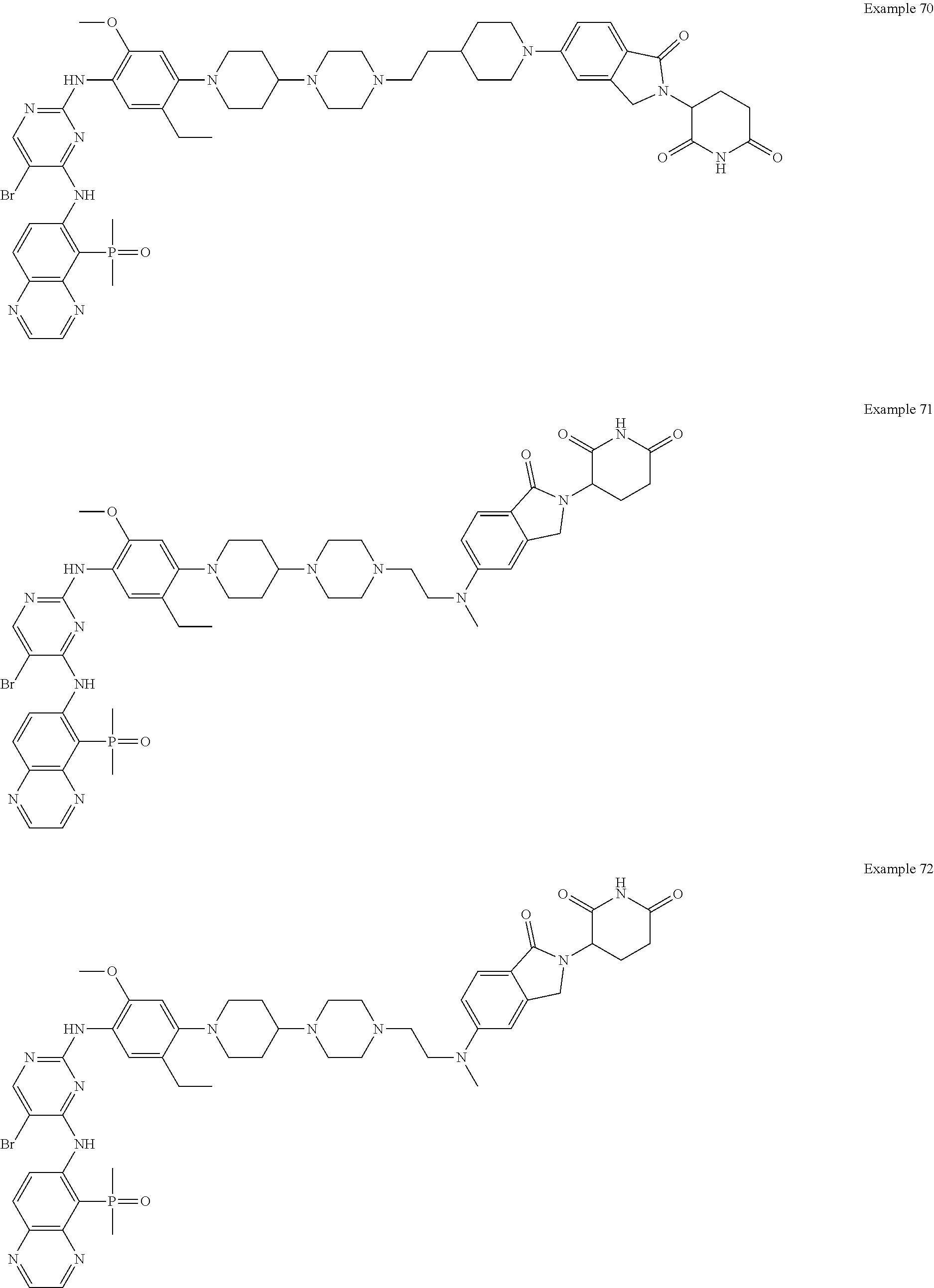 [SMILES: CCc1cc(Nc2ncc(Br)c(Nc3ccc4nccnc4c3P(C)(C)=O)n2)c(OC)cc1N1CCC(N2CCN(CCC3CCN(c4ccc5c(c4)CN(C4CCC(=O)NC4=O)C5=O)CC3)CC2)CC1 ; CCc1cc(Nc2ncc(Br)c(Nc3ccc4nccnc4c3P(C)(C)=O)n2)c(OC)cc1N1CCC(N2CCN(CCN(C)c3ccc4c(c3)CN(C3CCC(=O)NC3=O)C4=O)CC2)CC1 ; CCc1cc(Nc2ncc(Br)c(Nc3ccc4nccnc4c3P(C)(C)=O)n2)c(OC)cc1N1CCC(N2CCN(CCN(C)c3ccc4c(c3)CN(C3CCC(=O)NC3=O)C4=O)CC2)CC1]

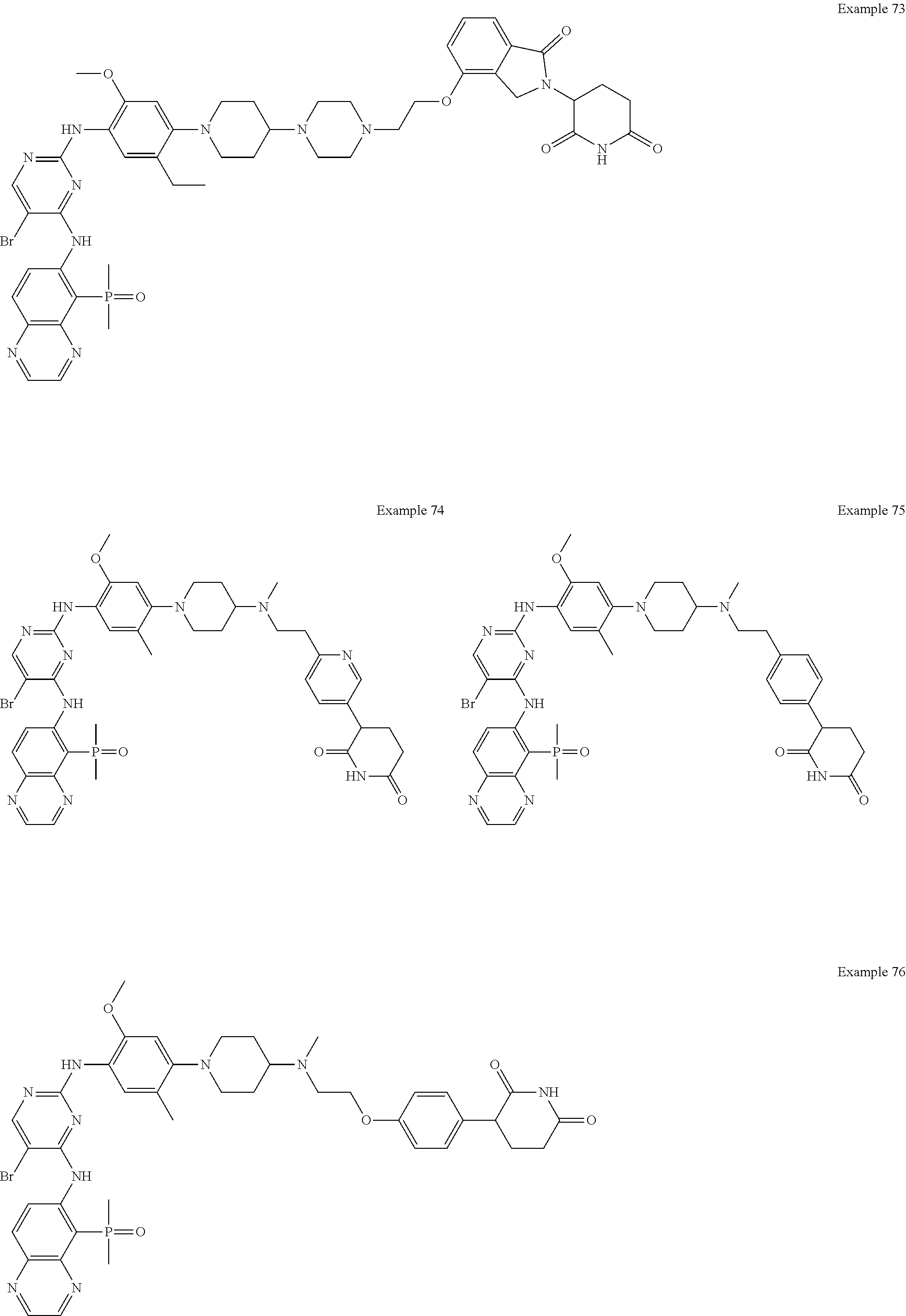 [SMILES: CCc1cc(Nc2ncc(Br)c(Nc3ccc4nccnc4c3P(C)(C)=O)n2)c(OC)cc1N1CCC(N2CCN(CCOc3cccc4c3CN(C3CCC(=O)NC3=O)C4=O)CC2)CC1 ; COc1cc(N2CCC(N(C)CCc3ccc(C4CCC(=O)NC4=O)cn3)CC2)c(C)cc1Nc1ncc(Br)c(Nc2ccc3nccnc3c2P(C)(C)=O)n1 ; COc1cc(N2CCC(N(C)CCc3ccc(C4CCC(=O)NC4=O)cc3)CC2)c(C)cc1Nc1ncc(Br)c(Nc2ccc3nccnc3c2P(C)(C)=O)n1 ; COc1cc(N2CCC(N(C)CCOc3ccc(C4CCC(=O)NC4=O)cc3)CC2)c(C)cc1Nc1ncc(Br)c(Nc2ccc3nccnc3c2P(C)(C)=O)n1]

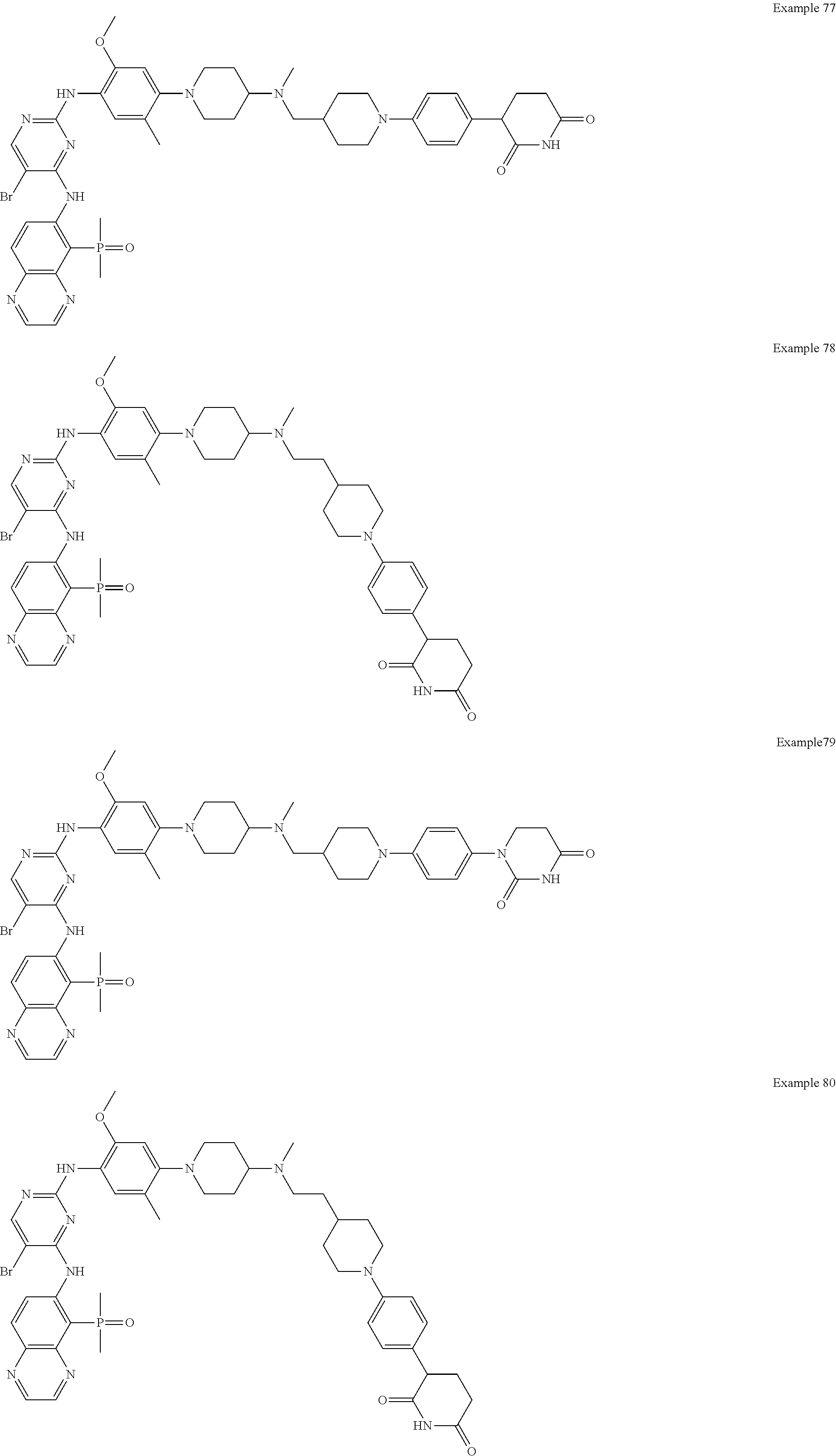 [SMILES: COc1cc(N2CCC(N(C)CC3CCN(c4ccc(C5CCC(=O)NC5=O)cc4)CC3)CC2)c(C)cc1Nc1ncc(Br)c(Nc2ccc3nccnc3c2P(C)(C)=O)n1 ; COc1cc(N2CCC(N(C)CCC3CCN(c4ccc(C5CCC(=O)NC5=O)cc4)CC3)CC2)c(C)cc1Nc1ncc(Br)c(Nc2ccc3nccnc3c2P(C)(C)=O)n1 ; COc1cc(N2CCC(N(C)CC3CCN(c4ccc(N5CCC(=O)NC5=O)cc4)CC3)CC2)c(C)cc1Nc1ncc(Br)c(Nc2ccc3nccnc3c2P(C)(C)=O)n1 ; COc1cc(N2CCC(N(C)CCC3CCN(c4ccc(C5CCC(=O)NC5=O)cc4)CC3)CC2)c(C)cc1Nc1ncc(Br)c(Nc2ccc3nccnc3c2P(C)(C)=O)n1]

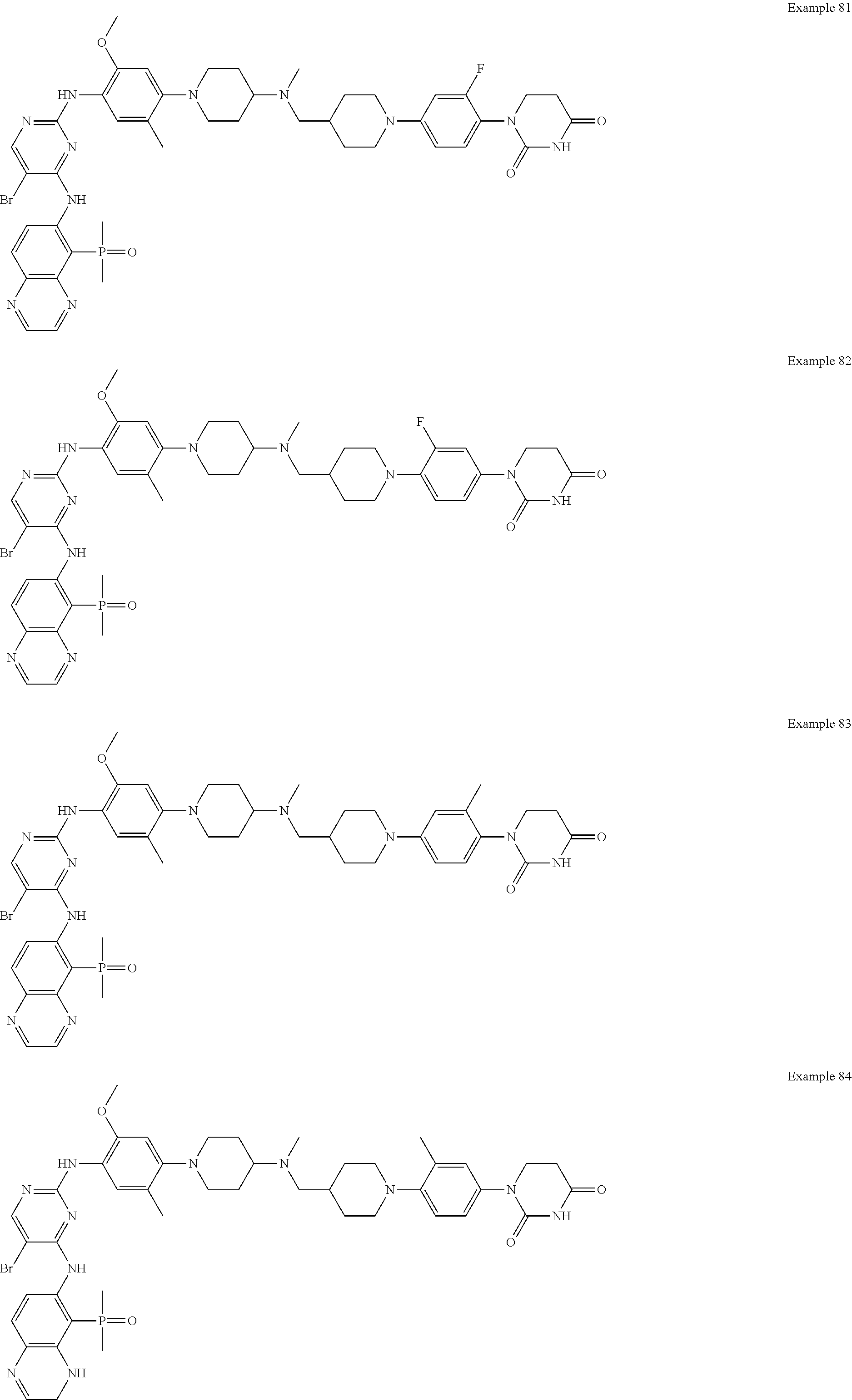 [SMILES: COc1cc(N2CCC(N(C)CC3CCN(c4ccc(N5CCC(=O)NC5=O)c(F)c4)CC3)CC2)c(C)cc1Nc1ncc(Br)c(Nc2ccc3nccnc3c2P(C)(C)=O)n1 ; COc1cc(N2CCC(N(C)CC3CCN(c4ccc(N5CCC(=O)NC5=O)cc4F)CC3)CC2)c(C)cc1Nc1ncc(Br)c(Nc2ccc3nccnc3c2P(C)(C)=O)n1 ; COc1cc(N2CCC(N(C)CC3CCN(c4ccc(N5CCC(=O)NC5=O)c(C)c4)CC3)CC2)c(C)cc1Nc1ncc(Br)c(Nc2ccc3nccnc3c2P(C)(C)=O)n1 ; COc1cc(N2CCC(N(C)CC3CCN(c4ccc(N5CCC(=O)NC5=O)cc4C)CC3)CC2)c(C)cc1Nc1ncc(Br)c(Nc2ccc3c(c2P(C)(C)=O)NCC=N3)n1]

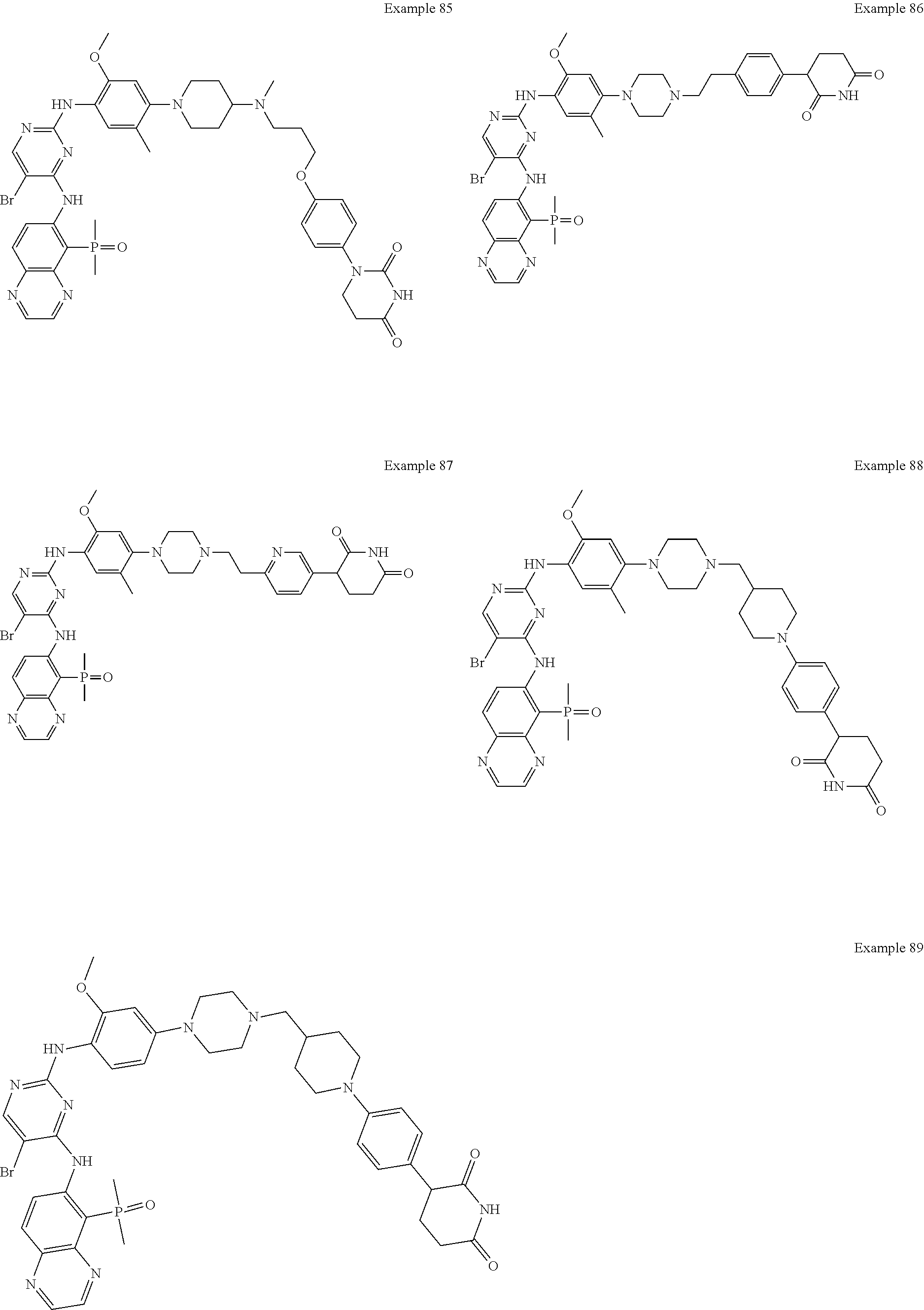 [SMILES: COc1cc(N2CCC(N(C)CCCOc3ccc(N4CCC(=O)NC4=O)cc3)CC2)c(C)cc1Nc1ncc(Br)c(Nc2ccc3nccnc3c2P(C)(C)=O)n1 ; COc1cc(N2CCN(CCc3ccc(C4CCC(=O)NC4=O)cc3)CC2)c(C)cc1Nc1ncc(Br)c(Nc2ccc3nccnc3c2P(C)(C)=O)n1 ; COc1cc(N2CCN(CCc3ccc(C4CCC(=O)NC4=O)cn3)CC2)c(C)cc1Nc1ncc(Br)c(Nc2ccc3nccnc3c2P(C)(C)=O)n1 ; COc1cc(N2CCN(CC3CCN(c4ccc(C5CCC(=O)NC5=O)cc4)CC3)CC2)c(C)cc1Nc1ncc(Br)c(Nc2ccc3nccnc3c2P(C)(C)=O)n1 ; COc1cc(N2CCN(CC3CCN(c4ccc(C5CCC(=O)NC5=O)cc4)CC3)CC2)ccc1Nc1ncc(Br)c(Nc2ccc3nccnc3c2P(C)(C)=O)n1]

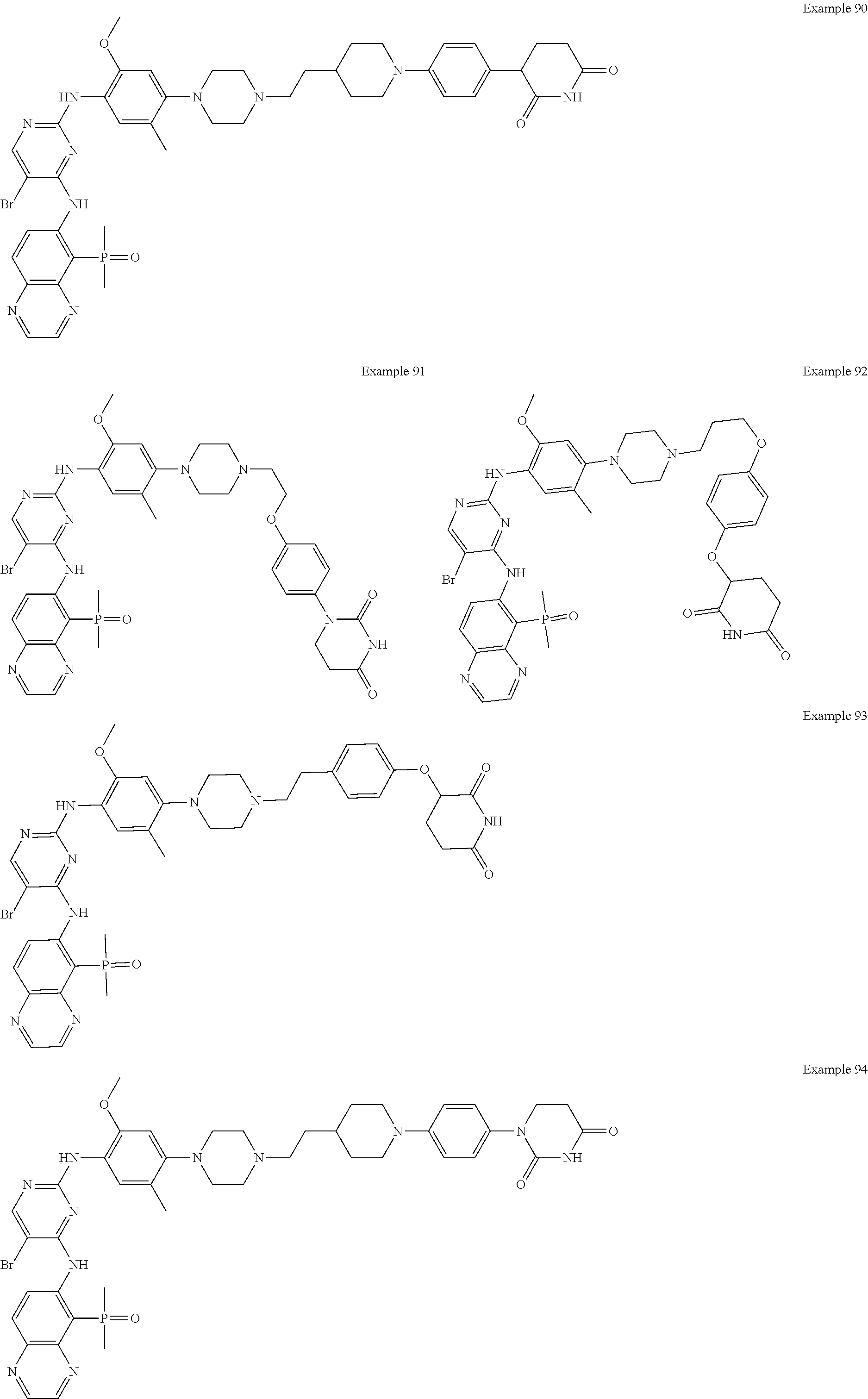 [SMILES: COc1cc(N2CCN(CCC3CCN(c4ccc(C5CCC(=O)NC5=O)cc4)CC3)CC2)c(C)cc1Nc1ncc(Br)c(Nc2ccc3nccnc3c2P(C)(C)=O)n1 ; COc1cc(N2CCN(CCOc3ccc(N4CCC(=O)NC4=O)cc3)CC2)c(C)cc1Nc1ncc(Br)c(Nc2ccc3nccnc3c2P(C)(C)=O)n1 ; COc1cc(N2CCN(CCCOc3ccc(OC4CCC(=O)NC4=O)cc3)CC2)c(C)cc1Nc1ncc(Br)c(Nc2ccc3nccnc3c2P(C)(C)=O)n1 ; COc1cc(N2CCN(CCc3ccc(OC4CCC(=O)NC4=O)cc3)CC2)c(C)cc1Nc1ncc(Br)c(Nc2ccc3nccnc3c2P(C)(C)=O)n1 ; COc1cc(N2CCN(CCC3CCN(c4ccc(N5CCC(=O)NC5=O)cc4)CC3)CC2)c(C)cc1Nc1ncc(Br)c(Nc2ccc3nccnc3c2P(C)(C)=O)n1]

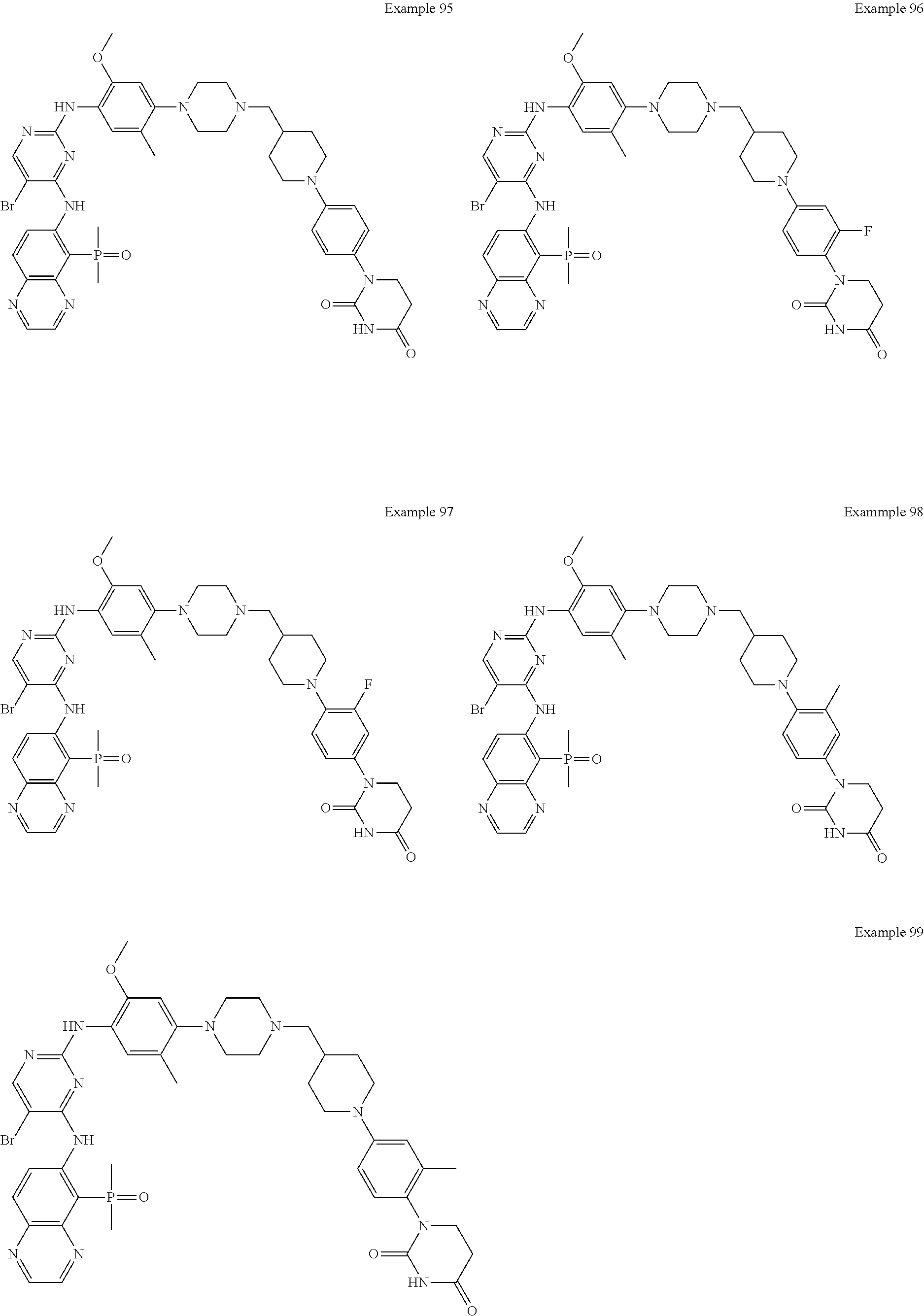

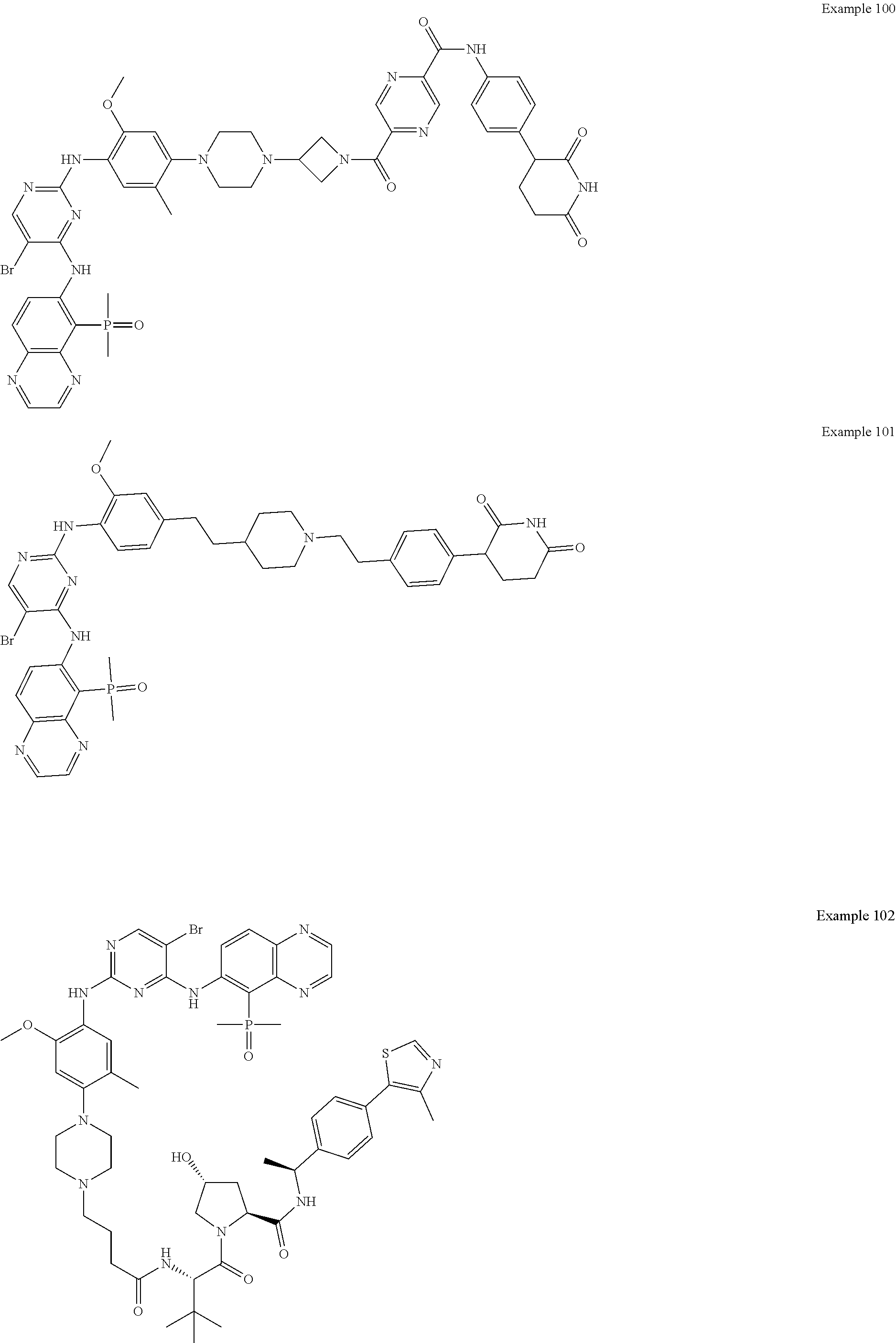

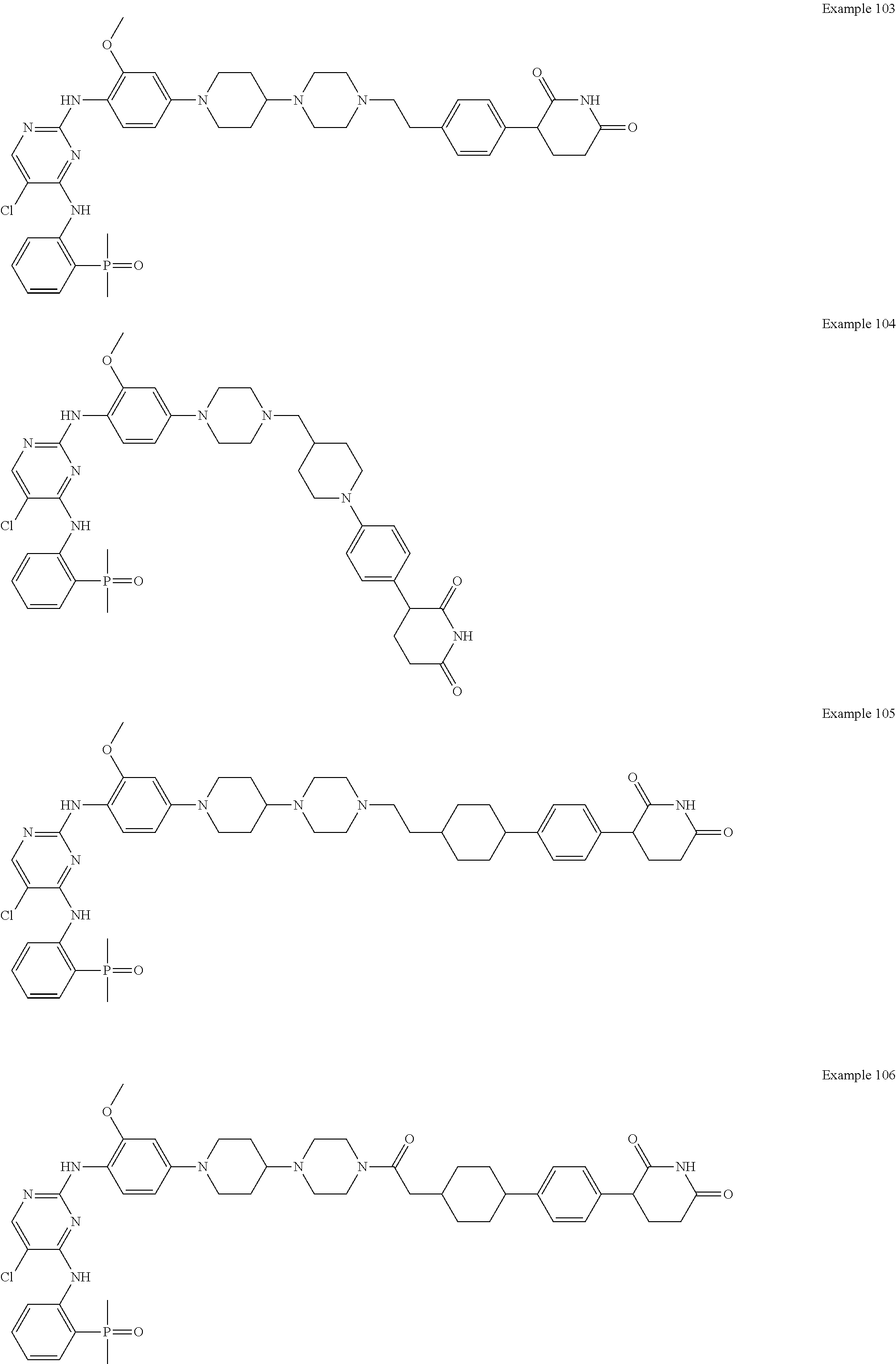

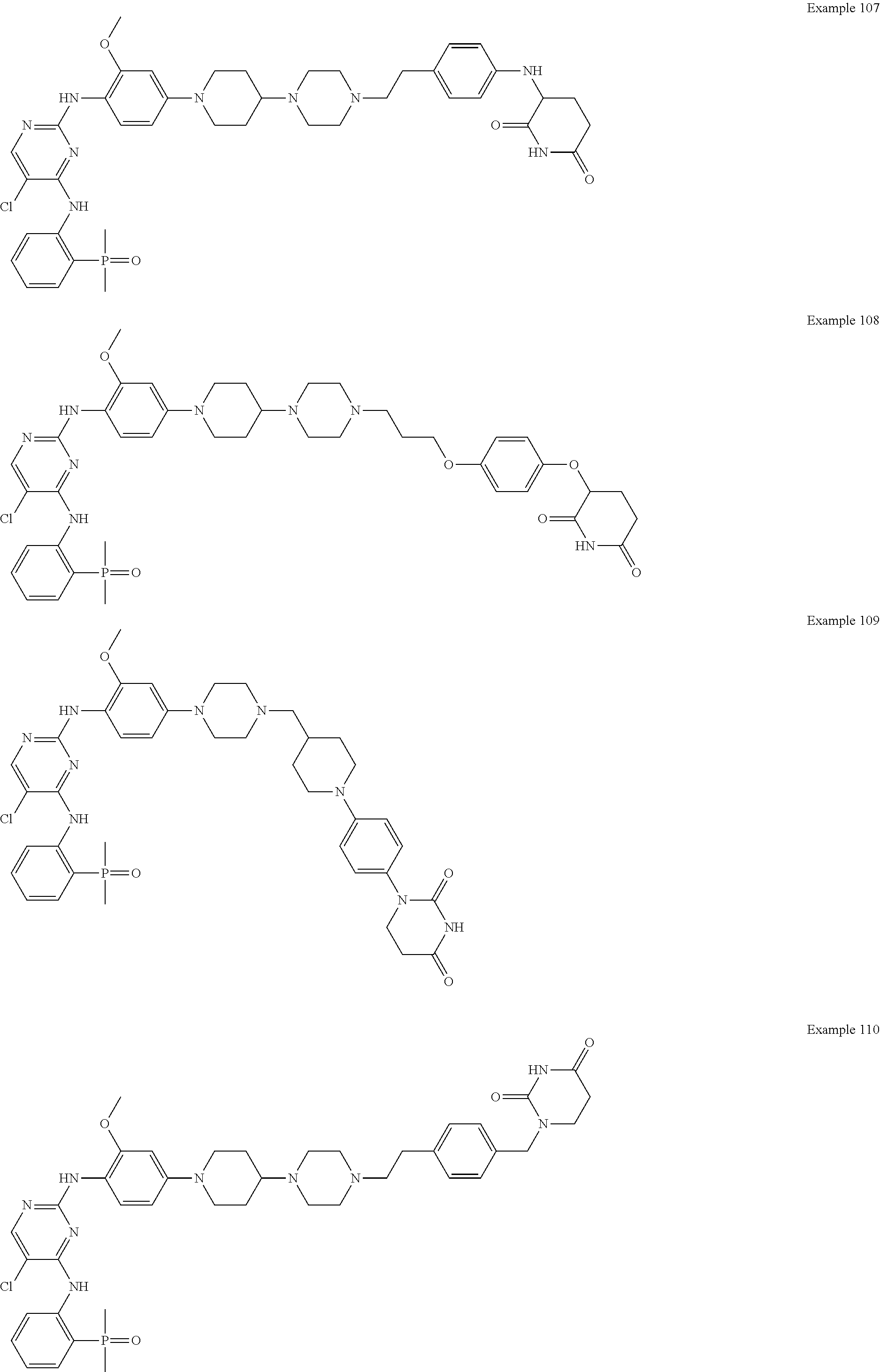

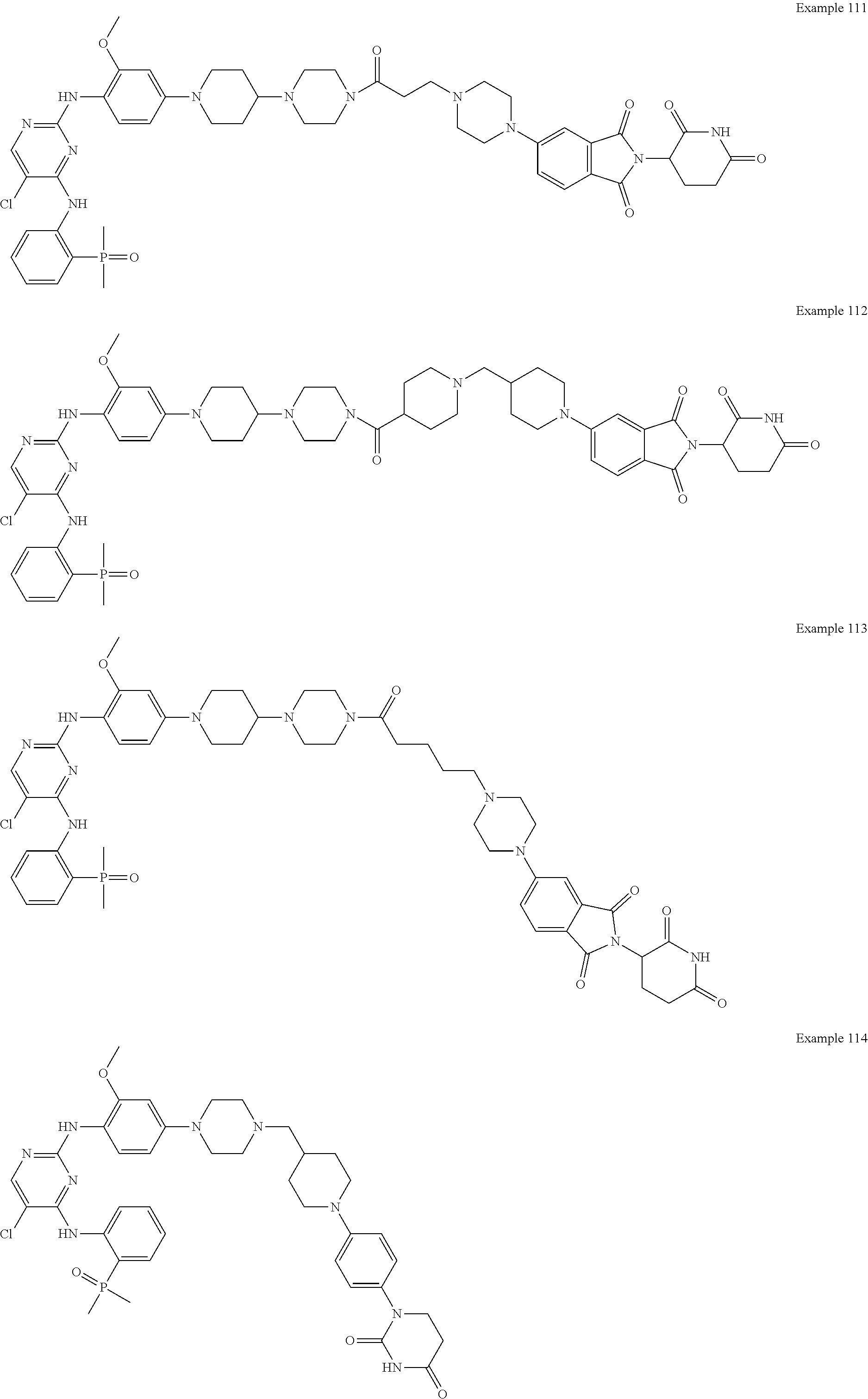

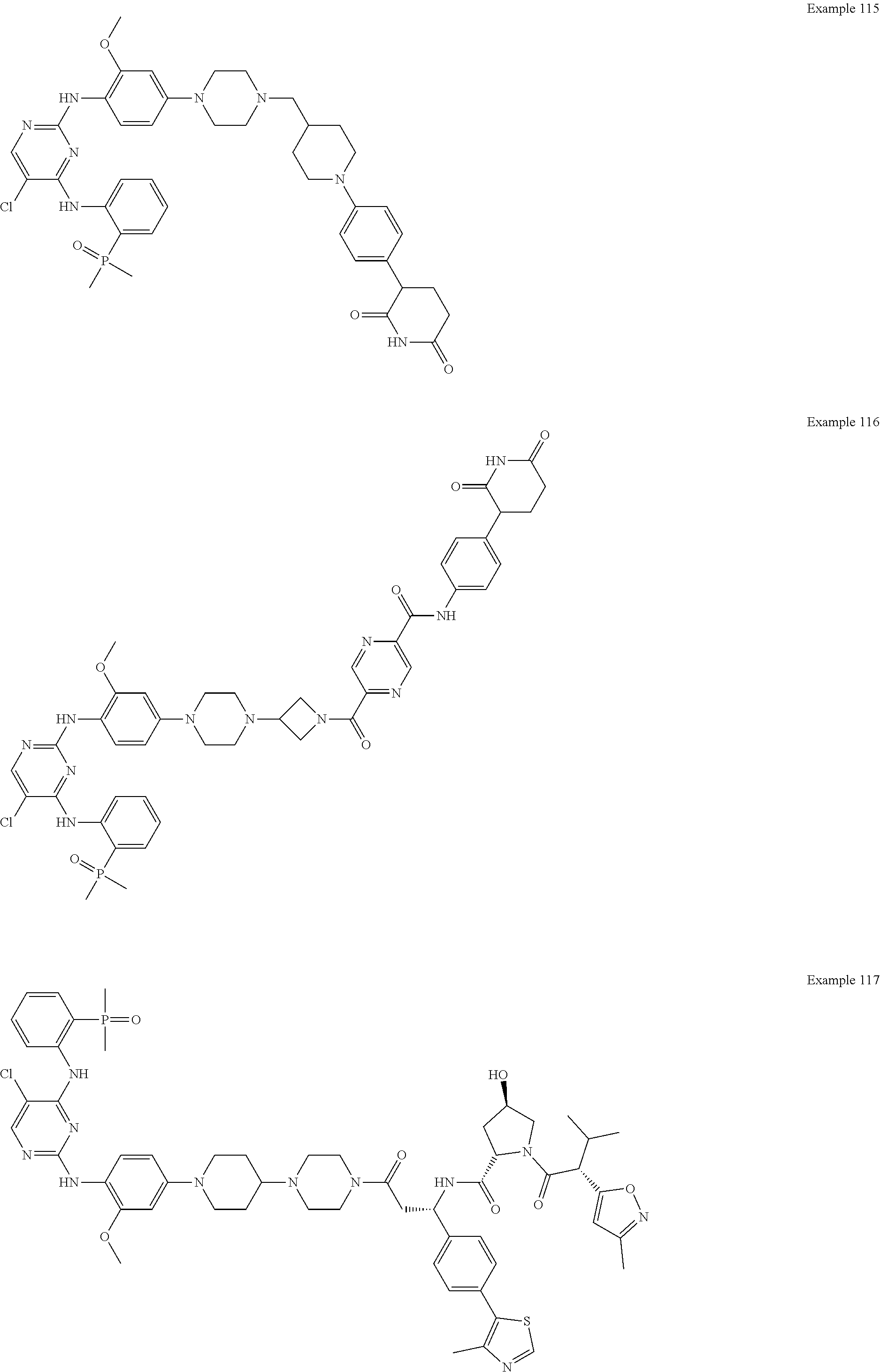

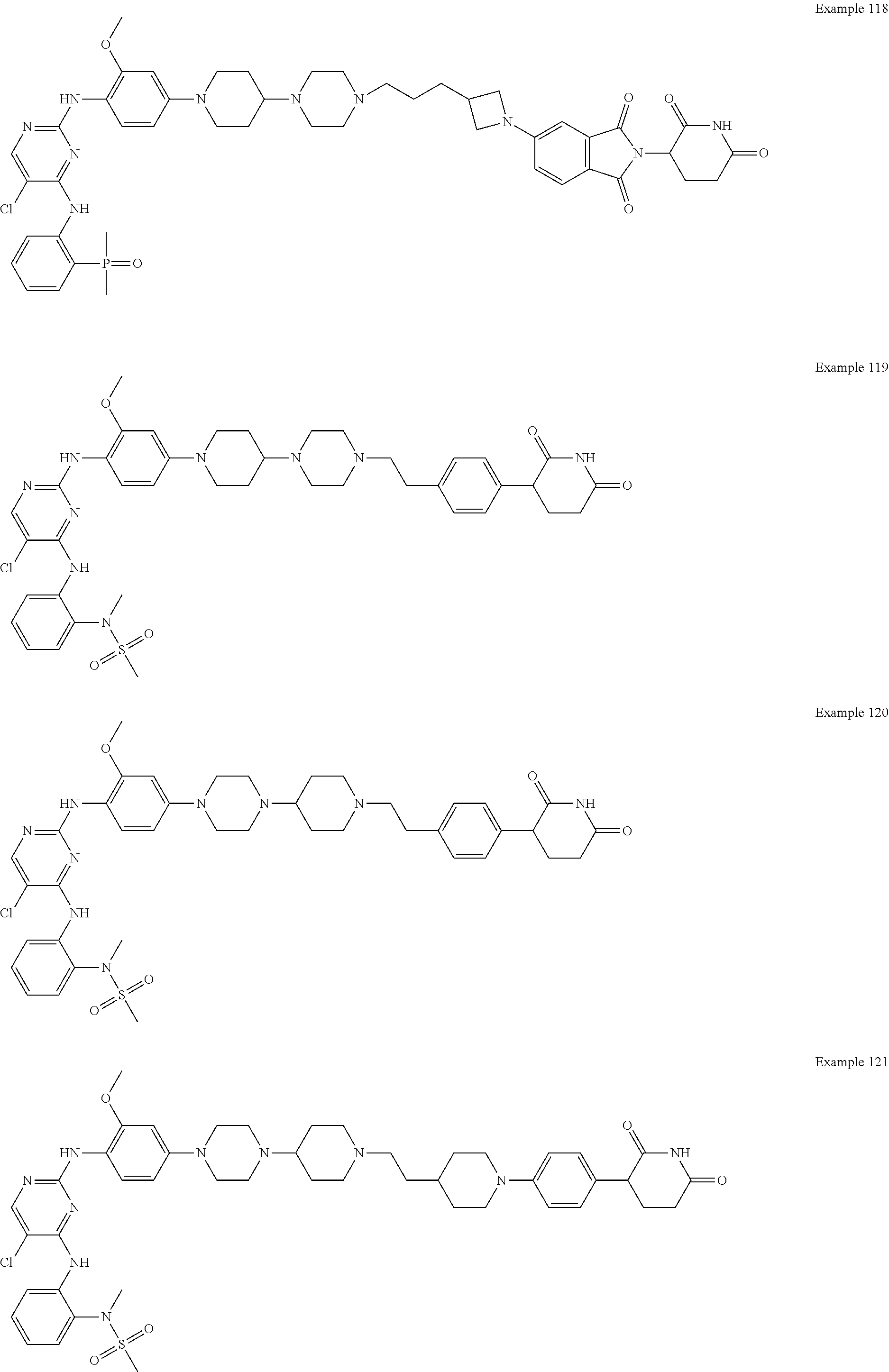 [SMILES: COc1cc(N2CCC(N3CCN(CCCC4CN(c5ccc6c(c5)C(=O)N(C5CCC(=O)NC5=O)C6=O)C4)CC3)CC2)ccc1Nc1ncc(Cl)c(Nc2ccccc2P(C)(C)=O)n1 ; COc1cc(N2CCC(N3CCN(CCc4ccc(C5CCC(=O)NC5=O)cc4)CC3)CC2)ccc1Nc1ncc(Cl)c(Nc2ccccc2N(C)S(C)(=O)=O)n1 ; COc1cc(N2CCN(C3CCN(CCc4ccc(C5CCC(=O)NC5=O)cc4)CC3)CC2)ccc1Nc1ncc(Cl)c(Nc2ccccc2N(C)S(C)(=O)=O)n1 ; COc1cc(N2CCN(C3CCN(CCC4CCN(c5ccc(C6CCC(=O)NC6=O)cc5)CC4)CC3)CC2)ccc1Nc1ncc(Cl)c(Nc2ccccc2N(C)S(C)(=O)=O)n1]

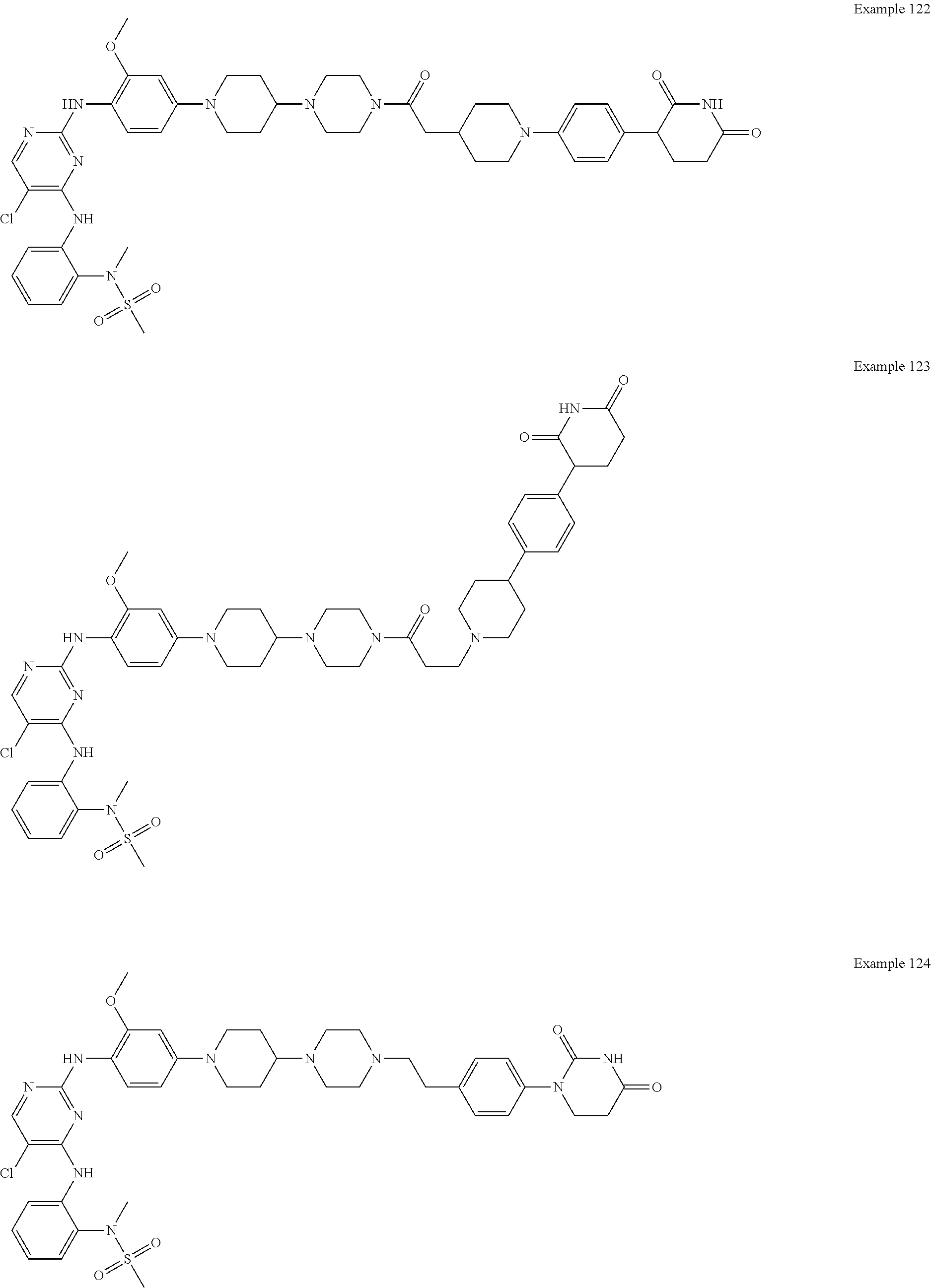 [SMILES: COc1cc(N2CCC(N3CCN(C(=O)CC4CCN(c5ccc(C6CCC(=O)NC6=O)cc5)CC4)CC3)CC2)ccc1Nc1ncc(Cl)c(Nc2ccccc2N(C)S(C)(=O)=O)n1 ; COc1cc(N2CCC(N3CCN(C(=O)CCN4CCC(c5ccc(C6CCC(=O)NC6=O)cc5)CC4)CC3)CC2)ccc1Nc1ncc(Cl)c(Nc2ccccc2N(C)S(C)(=O)=O)n1 ; COc1cc(N2CCC(N3CCN(CCc4ccc(N5CCC(=O)NC5=O)cc4)CC3)CC2)ccc1Nc1ncc(Cl)c(Nc2ccccc2N(C)S(C)(=O)=O)n1]

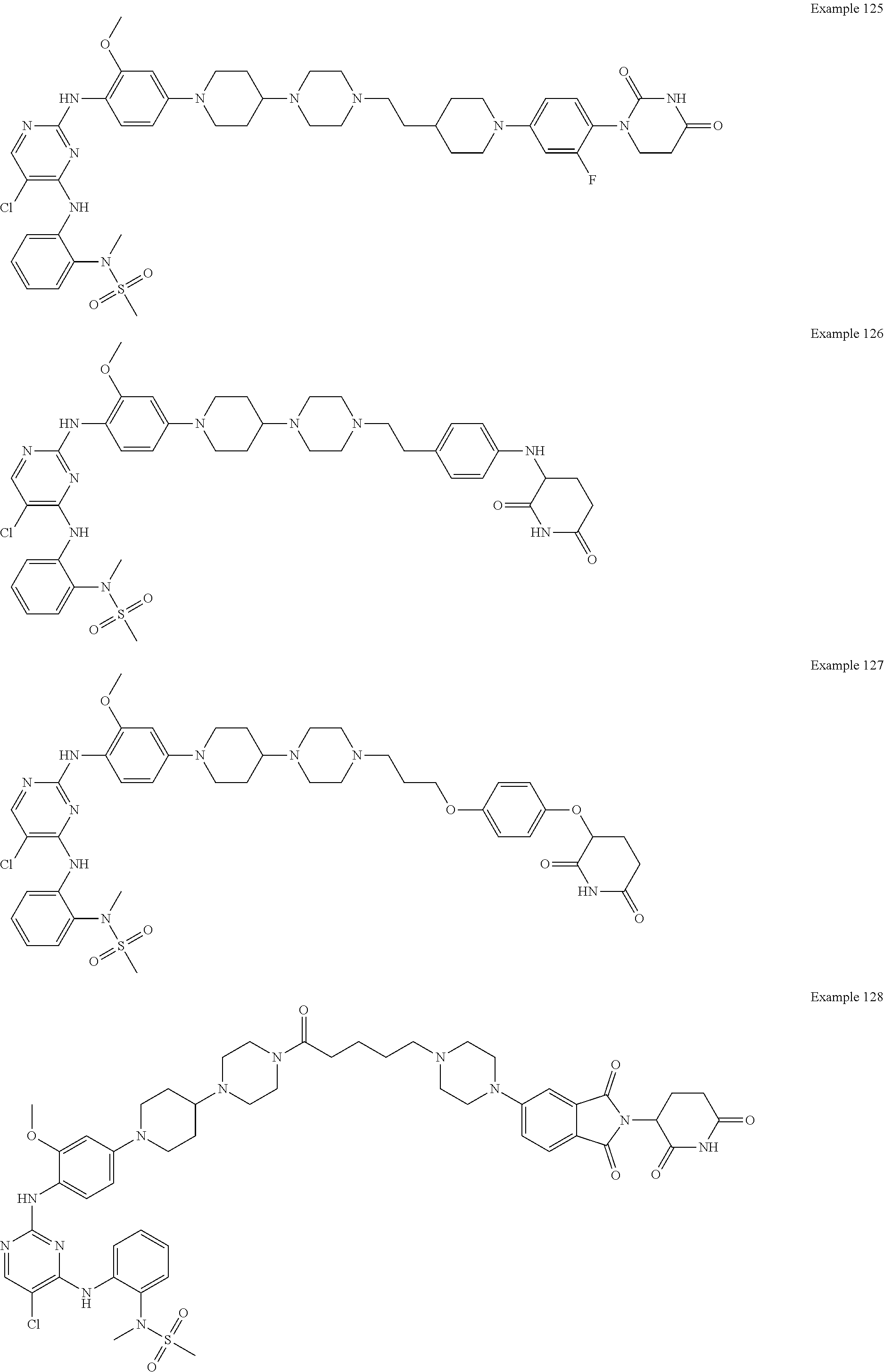

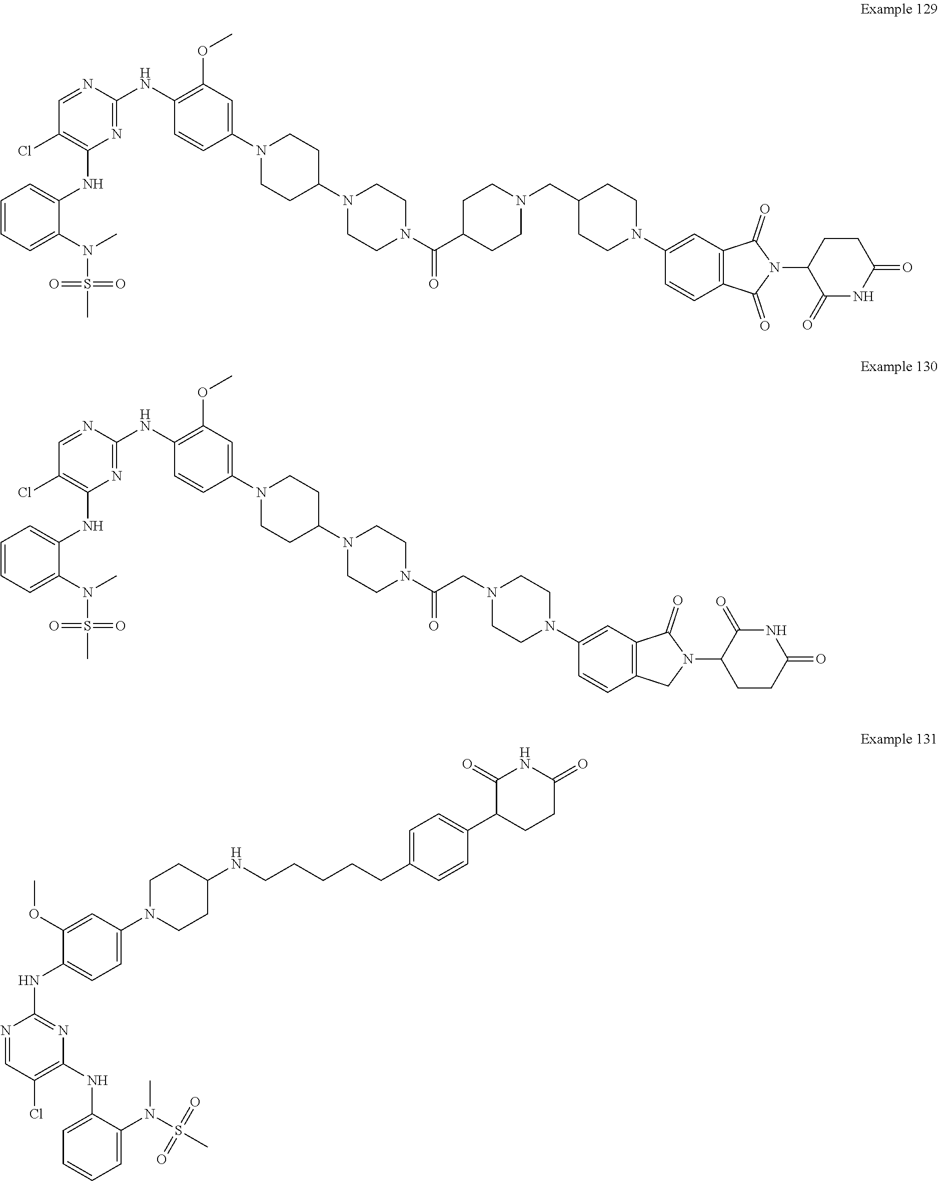 [SMILES: COc1cc(N2CCC(N3CCN(C(=O)C4CCN(CC5CCN(c6ccc7c(c6)C(=O)N(C6CCC(=O)NC6=O)C7=O)CC5)CC4)CC3)CC2)ccc1Nc1ncc(Cl)c(Nc2ccccc2N(C)S(C)(=O)=O)n1 ; COc1cc(N2CCC(N3CCN(C(=O)CN4CCN(c5ccc6c(c5)C(=O)N(C5CCC(=O)NC5=O)C6)CC4)CC3)CC2)ccc1Nc1ncc(Cl)c(Nc2ccccc2N(C)S(C)(=O)=O)n1 ; COc1cc(N2CCC(NCCCCCc3ccc(C4CCC(=O)NC4=O)cc3)CC2)ccc1Nc1ncc(Cl)c(Nc2ccccc2N(C)S(C)(=O)=O)n1]

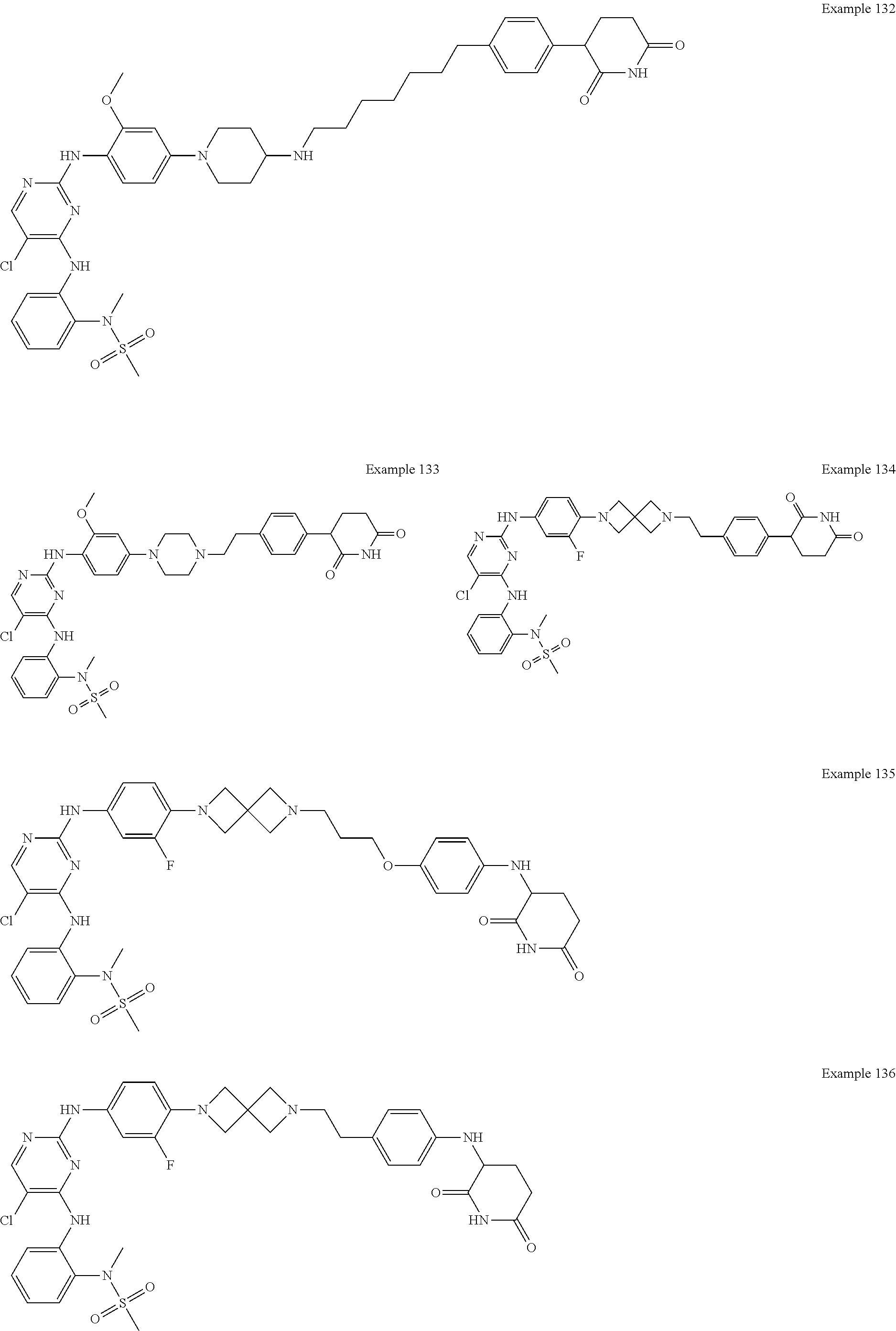 [SMILES: COc1cc(N2CCC(NCCCCCCCc3ccc(C4CCC(=O)NC4=O)cc3)CC2)ccc1Nc1ncc(Cl)c(Nc2ccccc2N(C)S(C)(=O)=O)n1 ; COc1cc(N2CCN(CCc3ccc(C4CCC(=O)NC4=O)cc3)CC2)ccc1Nc1ncc(Cl)c(Nc2ccccc2N(C)S(C)(=O)=O)n1 ; CN(c1ccccc1Nc1nc(Nc2ccc(N3CC4(CN(CCc5ccc(C6CCC(=O)NC6=O)cc5)C4)C3)c(F)c2)ncc1Cl)S(C)(=O)=O ; CN(c1ccccc1Nc1nc(Nc2ccc(N3CC4(CN(CCCOc5ccc(NC6CCC(=O)NC6=O)cc5)C4)C3)c(F)c2)ncc1Cl)S(C)(=O)=O ; CN(c1ccccc1Nc1nc(Nc2ccc(N3CC4(CN(CCc5ccc(NC6CCC(=O)NC6=O)cc5)C4)C3)c(F)c2)ncc1Cl)S(C)(=O)=O]

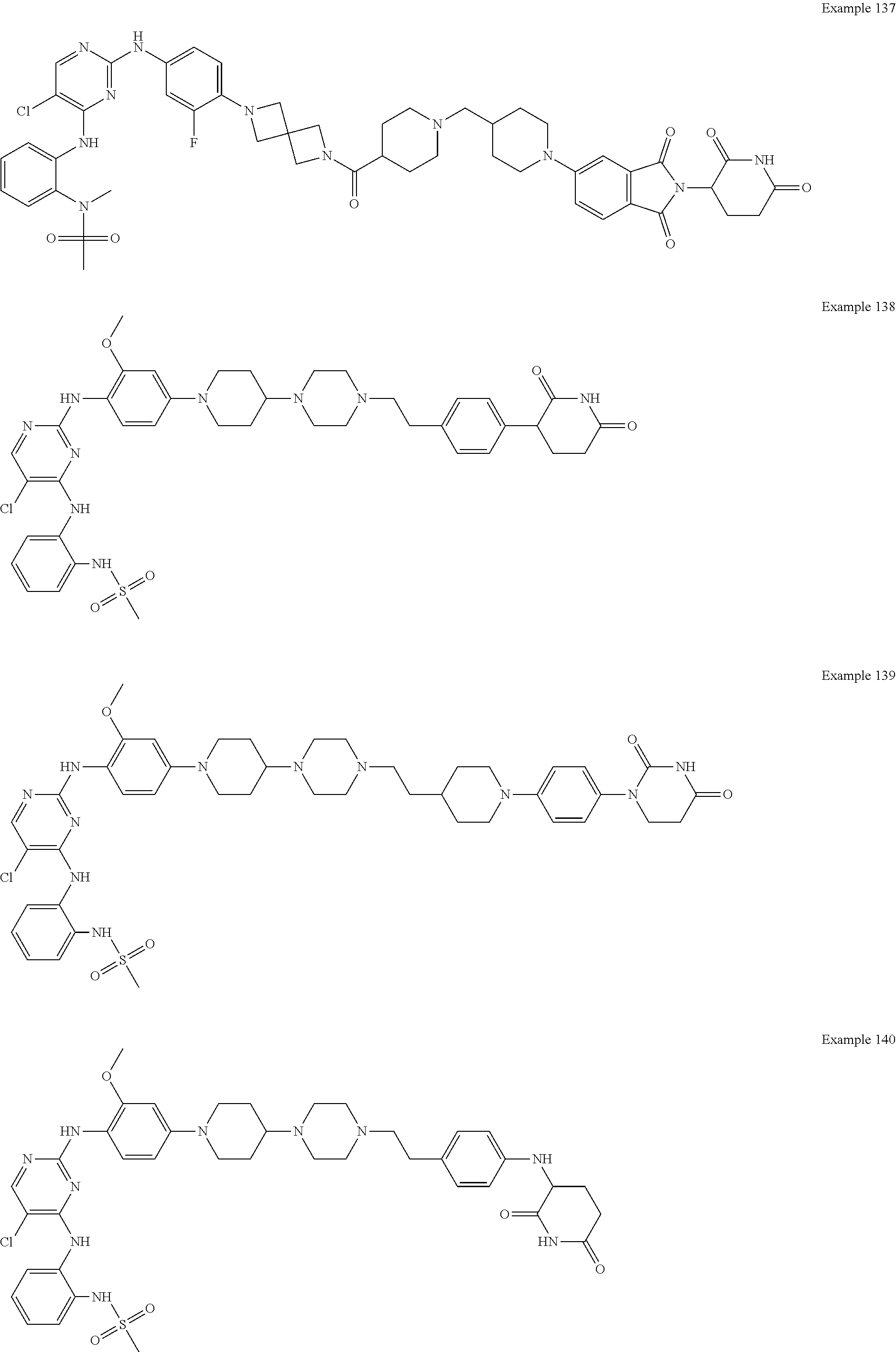

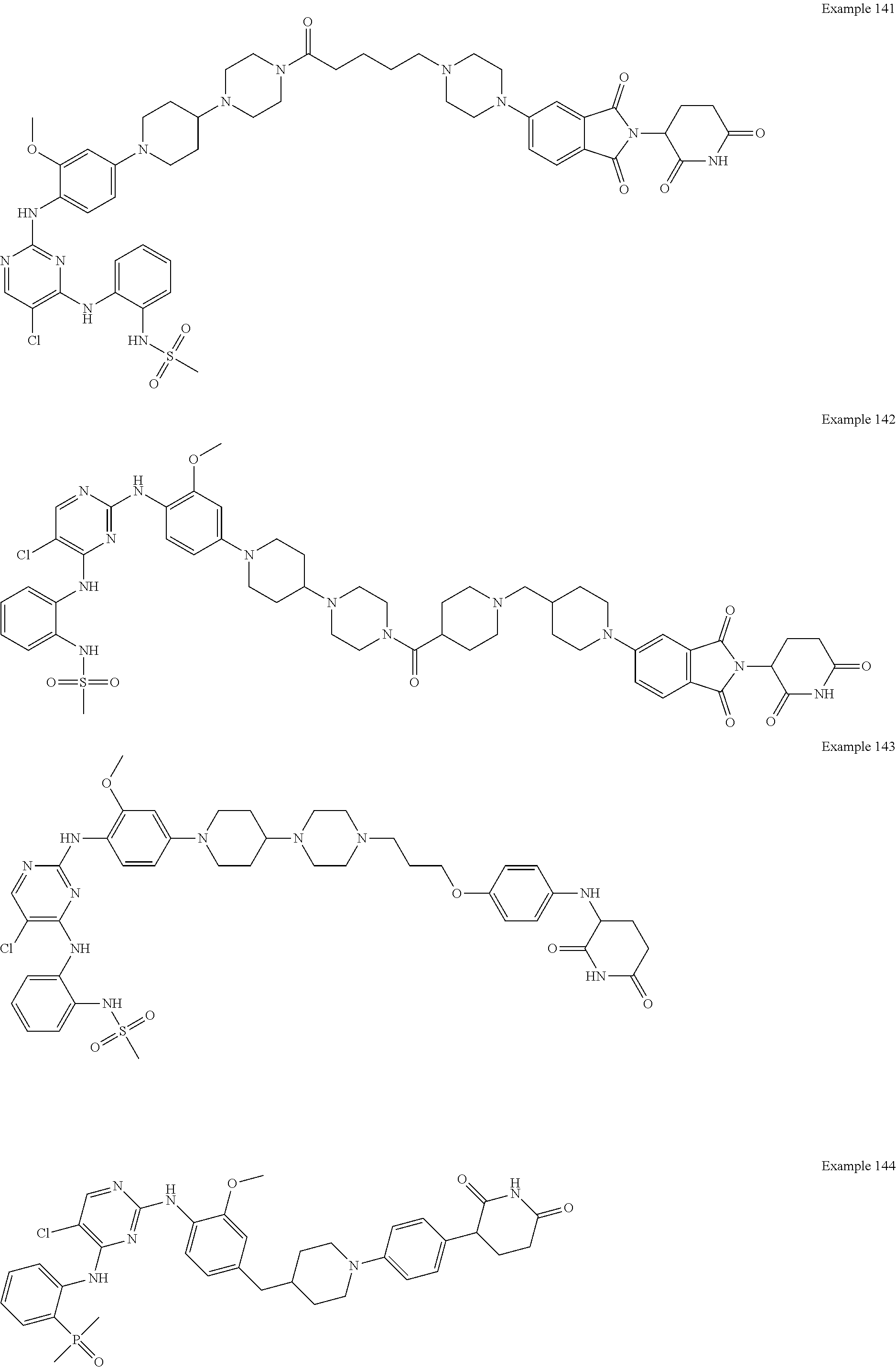 [SMILES: COc1cc(N2CCC(N3CCN(C(=O)CCCCN4CCN(c5ccc6c(c5)C(=O)N(C5CCC(=O)NC5=O)C6=O)CC4)CC3)CC2)ccc1Nc1ncc(Cl)c(Nc2ccccc2NS(C)(=O)=O)n1 ; COc1cc(N2CCC(N3CCN(C(=O)C4CCN(CC5CCN(c6ccc7c(c6)C(=O)N(C6CCC(=O)NC6=O)C7=O)CC5)CC4)CC3)CC2)ccc1Nc1ncc(Cl)c(Nc2ccccc2NS(C)(=O)=O)n1 ; COc1cc(N2CCC(N3CCN(CCCOc4ccc(NC5CCC(=O)NC5=O)cc4)CC3)CC2)ccc1Nc1ncc(Cl)c(Nc2ccccc2NS(C)(=O)=O)n1 ; COc1cc(CC2CCN(c3ccc(C4CCC(=O)NC4=O)cc3)CC2)ccc1Nc1ncc(Cl)c(Nc2ccccc2P(C)(C)=O)n1]

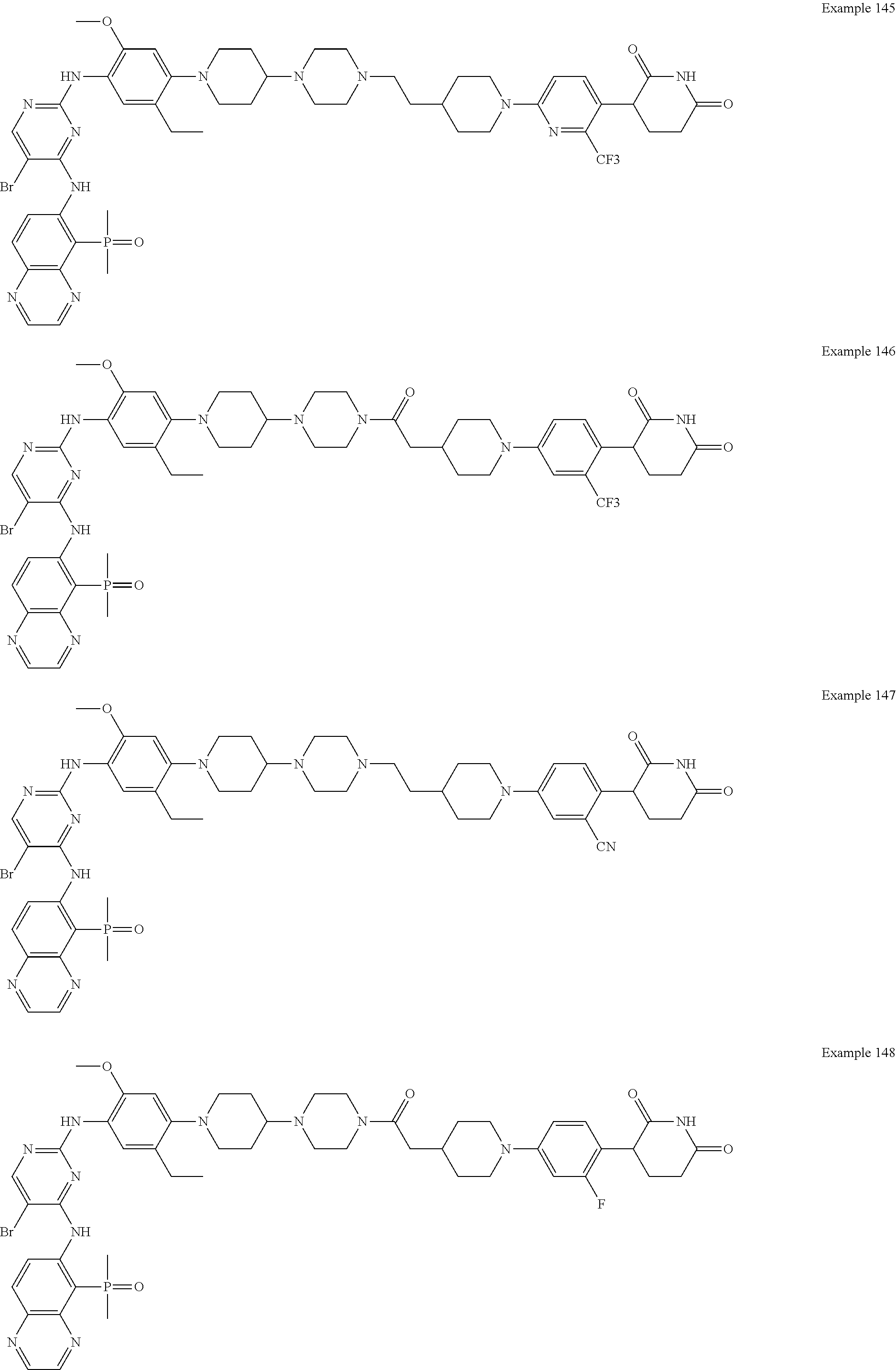

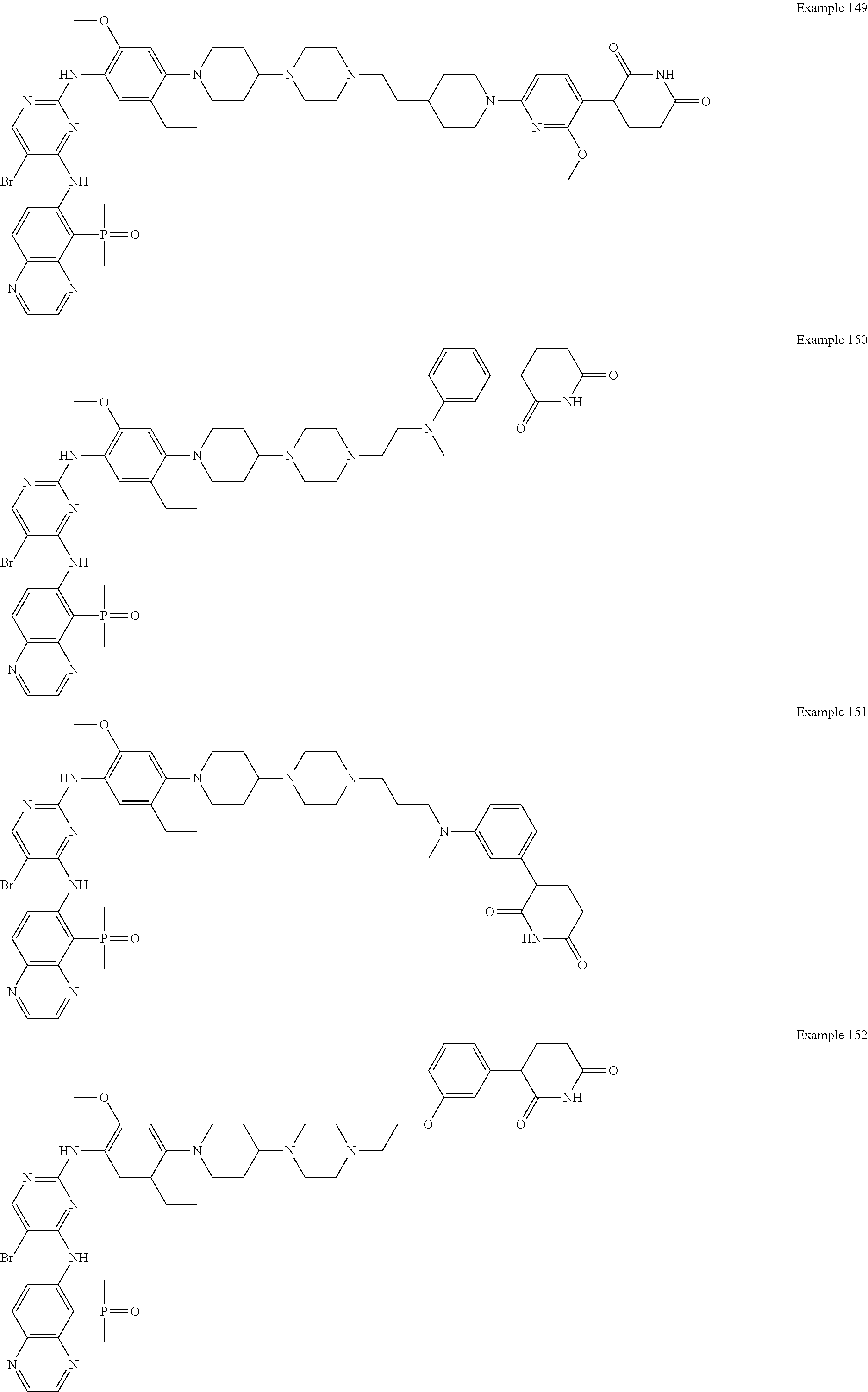

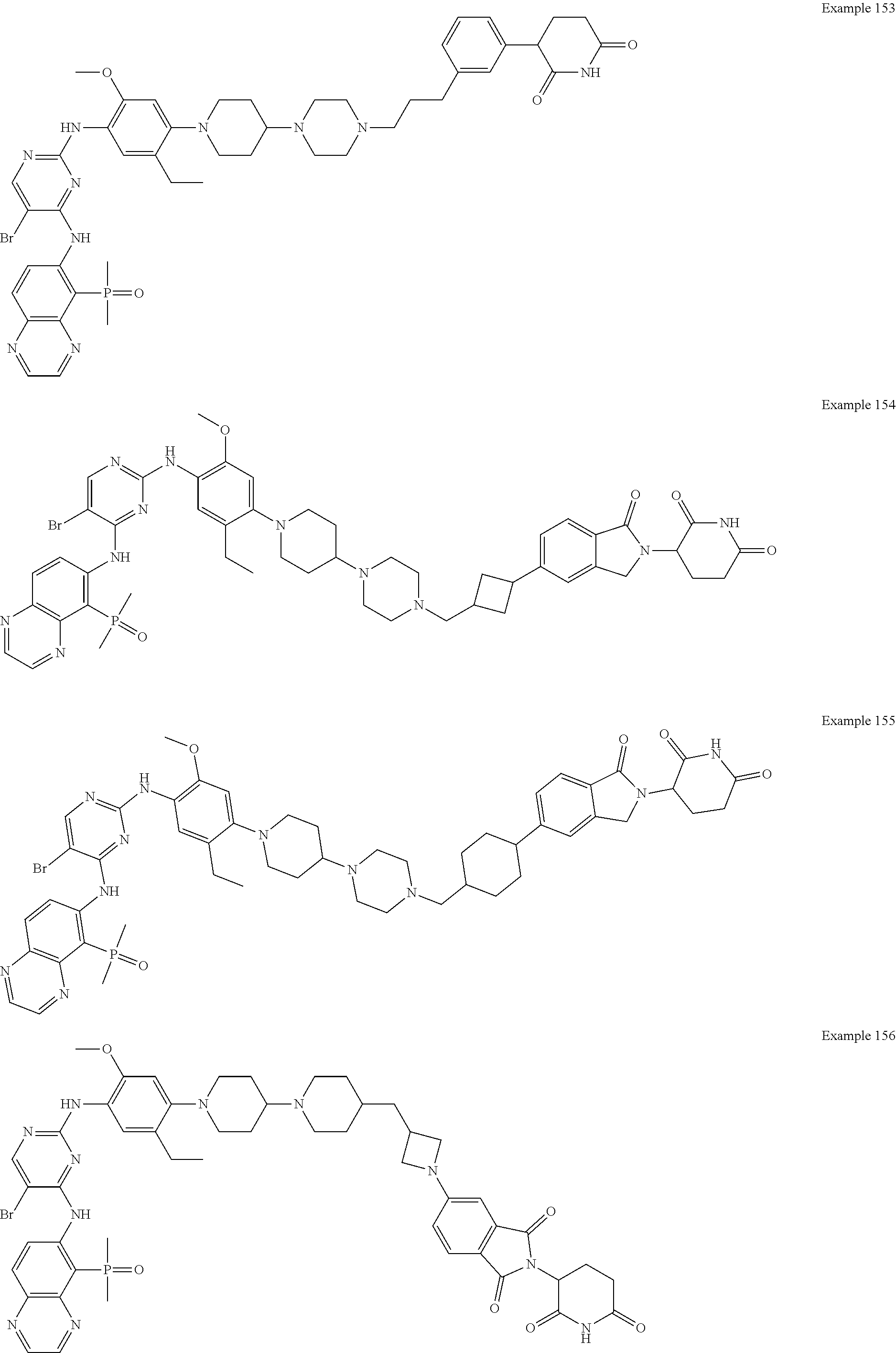 [SMILES: CCc1cc(Nc2ncc(Br)c(Nc3ccc4nccnc4c3P(C)(C)=O)n2)c(OC)cc1N1CCC(N2CCN(CCCc3cccc(C4CCC(=O)NC4=O)c3)CC2)CC1 ; CCc1cc(Nc2ncc(Br)c(Nc3ccc4nccnc4c3P(C)(C)=O)n2)c(OC)cc1N1CCC(N2CCN(CC3CC(c4ccc5c(c4)CN(C4CCC(=O)NC4=O)C5=O)C3)CC2)CC1 ; CCc1cc(Nc2ncc(Br)c(Nc3ccc4nccnc4c3P(C)(C)=O)n2)c(OC)cc1N1CCC(N2CCN(CC3CCC(c4ccc5c(c4)CN(C4CCC(=O)NC4=O)C5=O)CC3)CC2)CC1 ; CCc1cc(Nc2ncc(Br)c(Nc3ccc4nccnc4c3P(C)(C)=O)n2)c(OC)cc1N1CCC(N2CCC(CC3CN(c4ccc5c(c4)C(=O)N(C4CCC(=O)NC4=O)C5=O)C3)CC2)CC1]

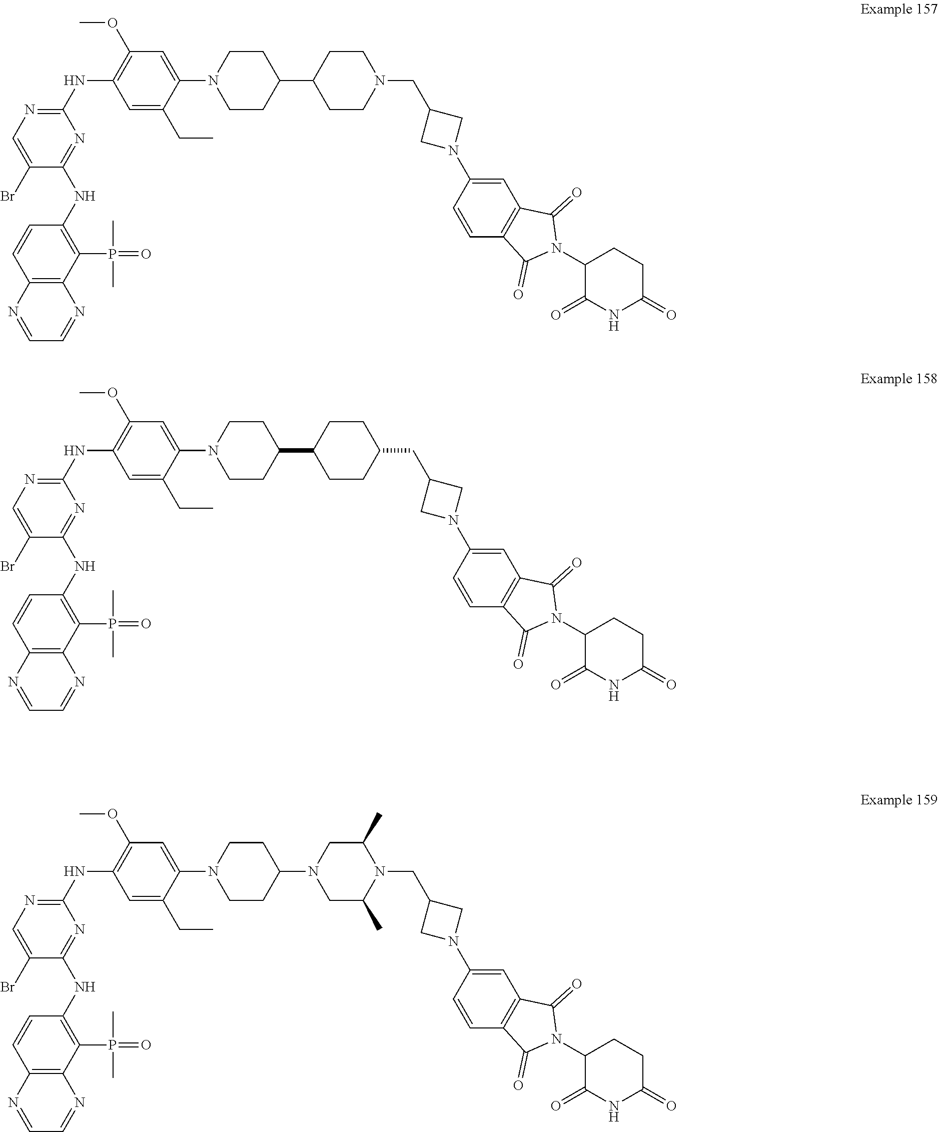

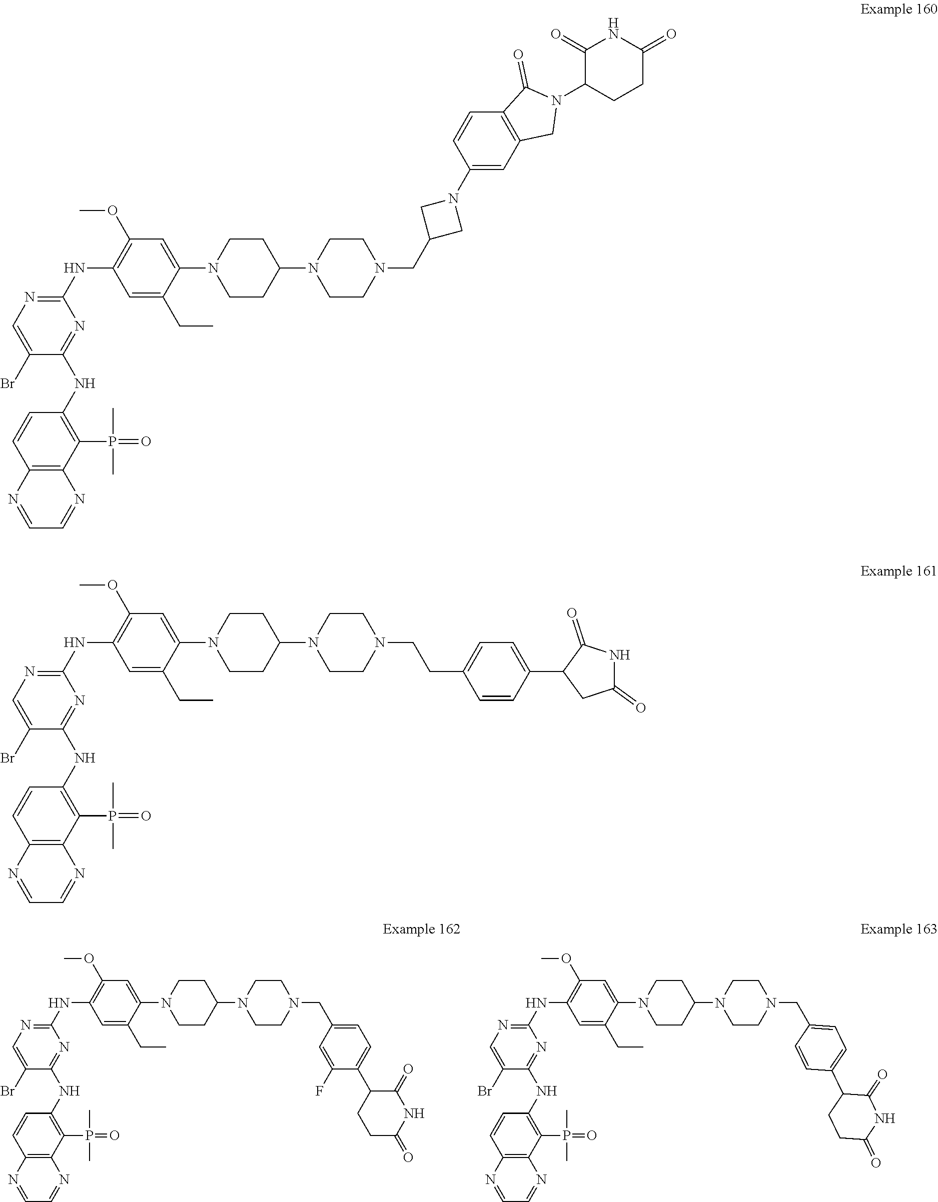 [SMILES: CCc1cc(Nc2ncc(Br)c(Nc3ccc4nccnc4c3P(C)(C)=O)n2)c(OC)cc1N1CCC(N2CCN(CC3CN(c4ccc5c(c4)CN(C4CCC(=O)NC4=O)C5=O)C3)CC2)CC1 ; CCc1cc(Nc2ncc(Br)c(Nc3ccc4nccnc4c3P(C)(C)=O)n2)c(OC)cc1N1CCC(N2CCN(CCc3ccc(C4CC(=O)NC4=O)cc3)CC2)CC1 ; CCc1cc(Nc2ncc(Br)c(Nc3ccc4nccnc4c3P(C)(C)=O)n2)c(OC)cc1N1CCC(N2CCN(Cc3ccc(C4CCC(=O)NC4=O)c(F)c3)CC2)CC1 ; CCc1cc(Nc2ncc(Br)c(Nc3ccc4nccnc4c3P(C)(C)=O)n2)c(OC)cc1N1CCC(N2CCN(Cc3ccc(C4CCC(=O)NC4=O)cc3)CC2)CC1]

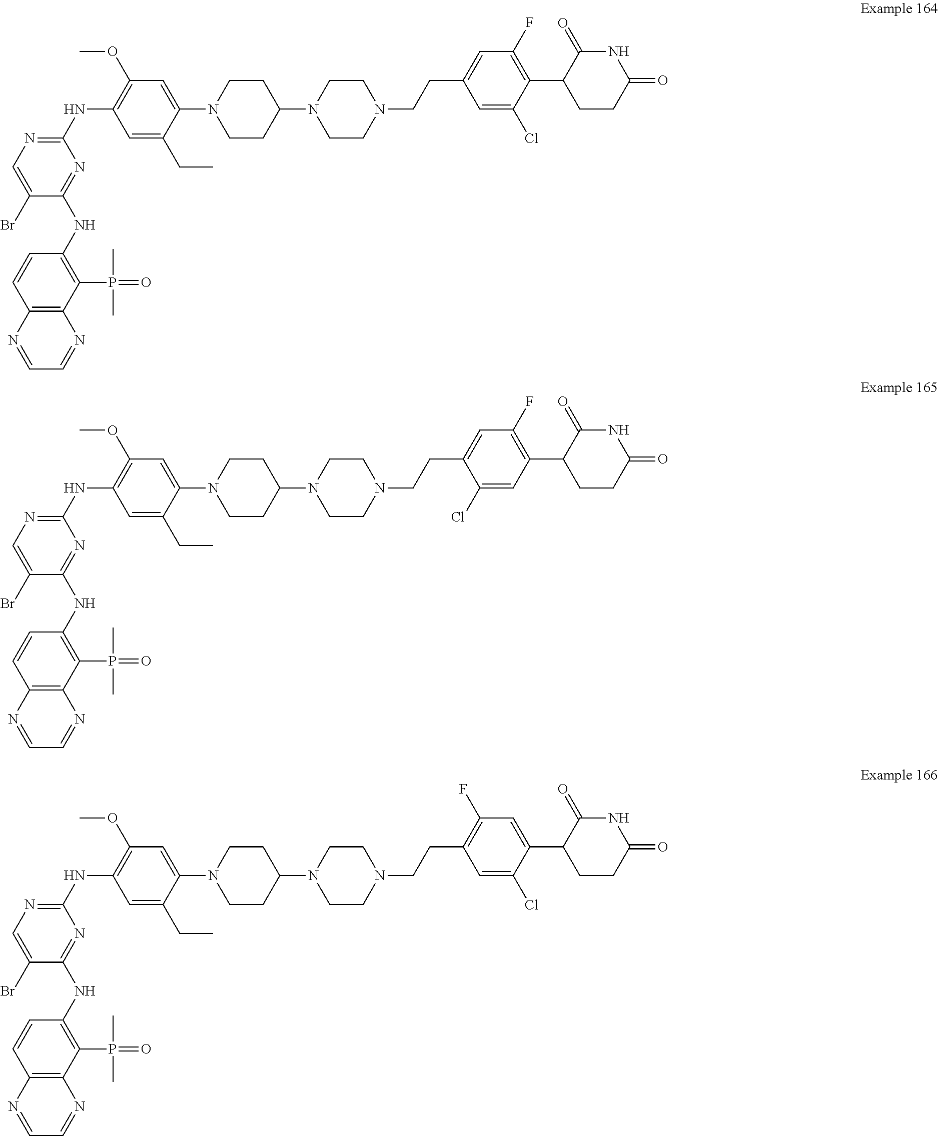 [SMILES: CCc1cc(Nc2ncc(Br)c(Nc3ccc4nccnc4c3P(C)(C)=O)n2)c(OC)cc1N1CCC(N2CCN(CCc3cc(F)c(C4CCC(=O)NC4=O)c(Cl)c3)CC2)CC1 ; CCc1cc(Nc2ncc(Br)c(Nc3ccc4nccnc4c3P(C)(C)=O)n2)c(OC)cc1N1CCC(N2CCN(CCc3cc(F)c(C4CCC(=O)NC4=O)cc3Cl)CC2)CC1 ; CCc1cc(Nc2ncc(Br)c(Nc3ccc4nccnc4c3P(C)(C)=O)n2)c(OC)cc1N1CCC(N2CCN(CCc3cc(Cl)c(C4CCC(=O)NC4=O)cc3F)CC2)CC1]

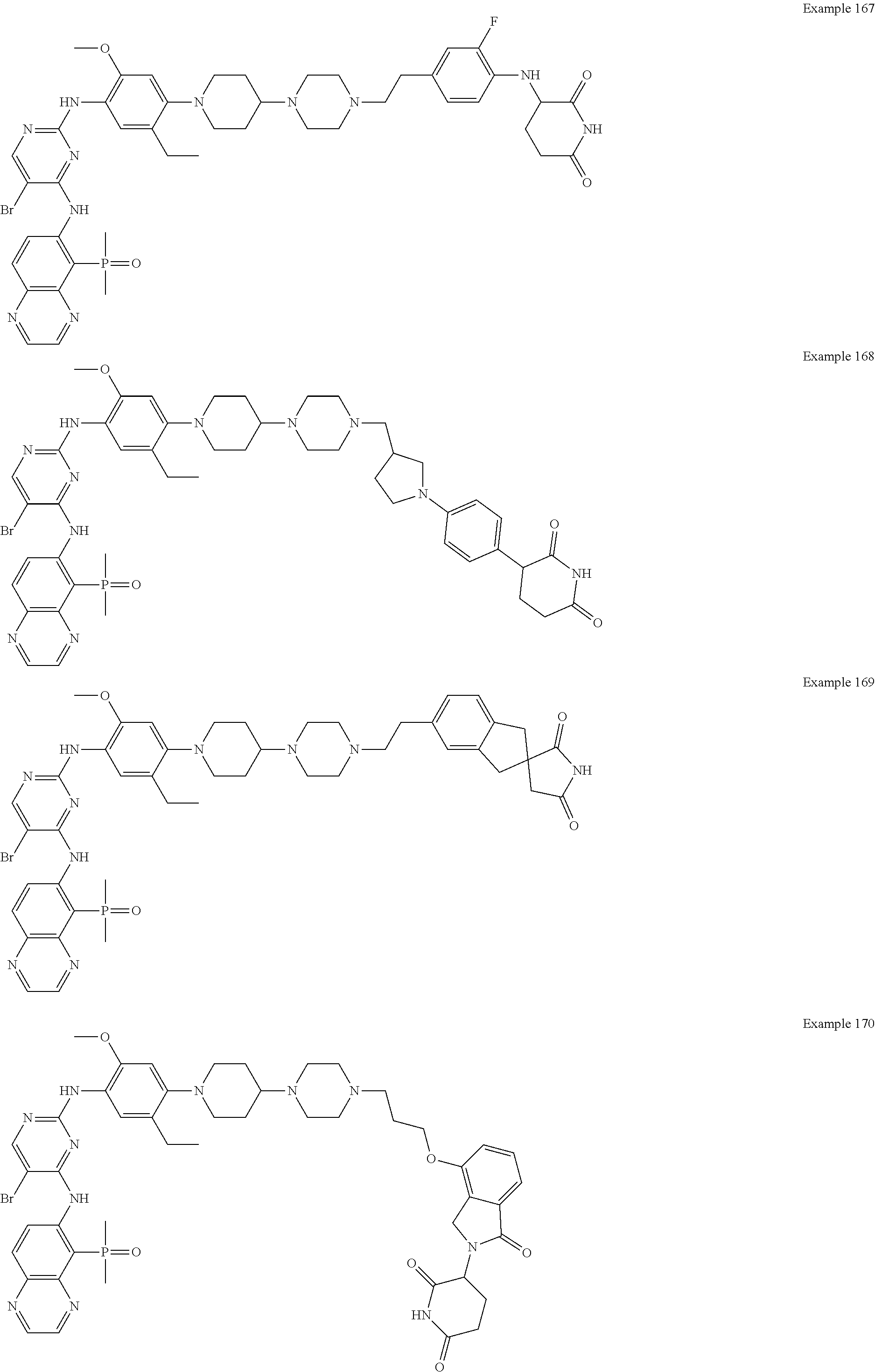

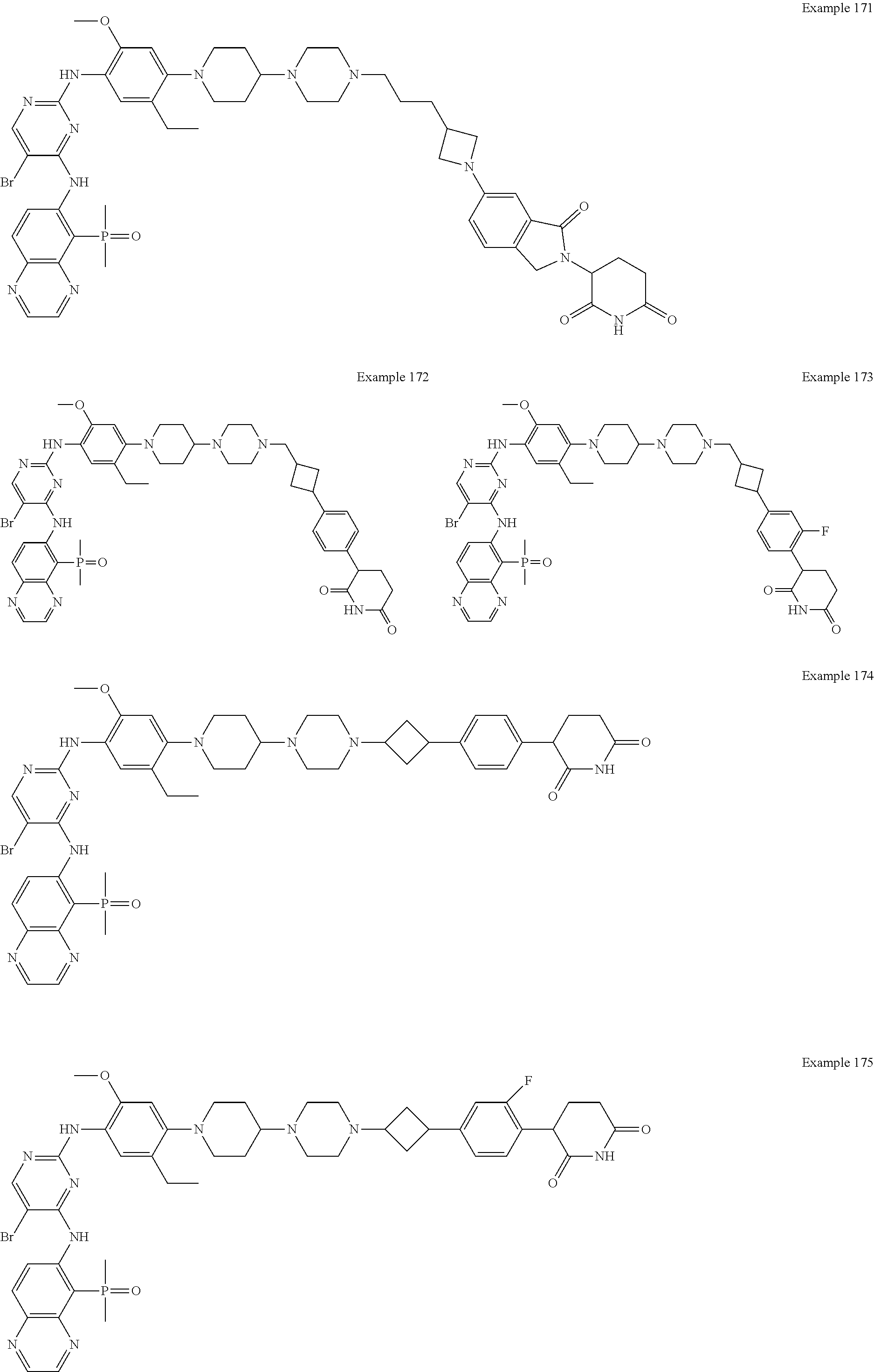

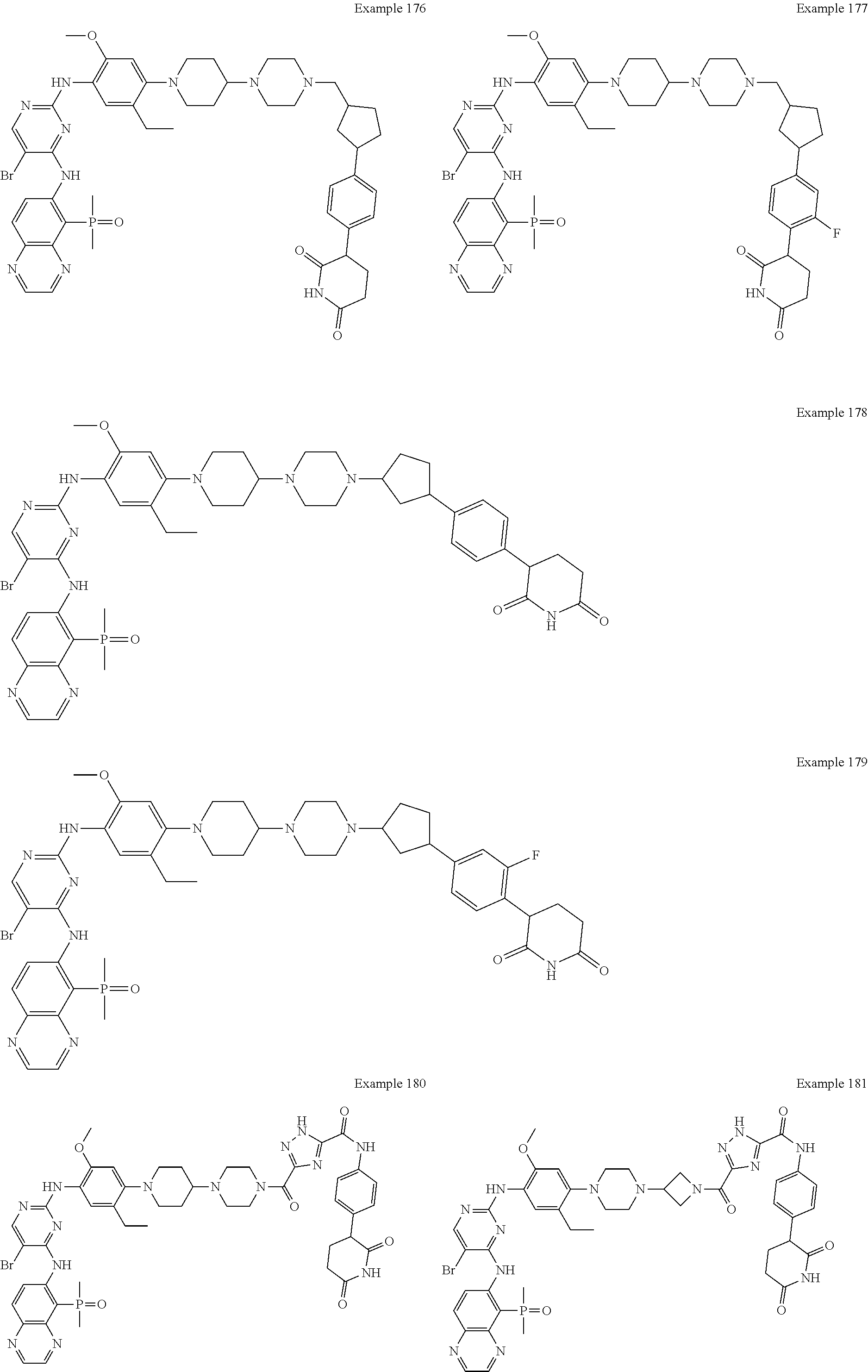

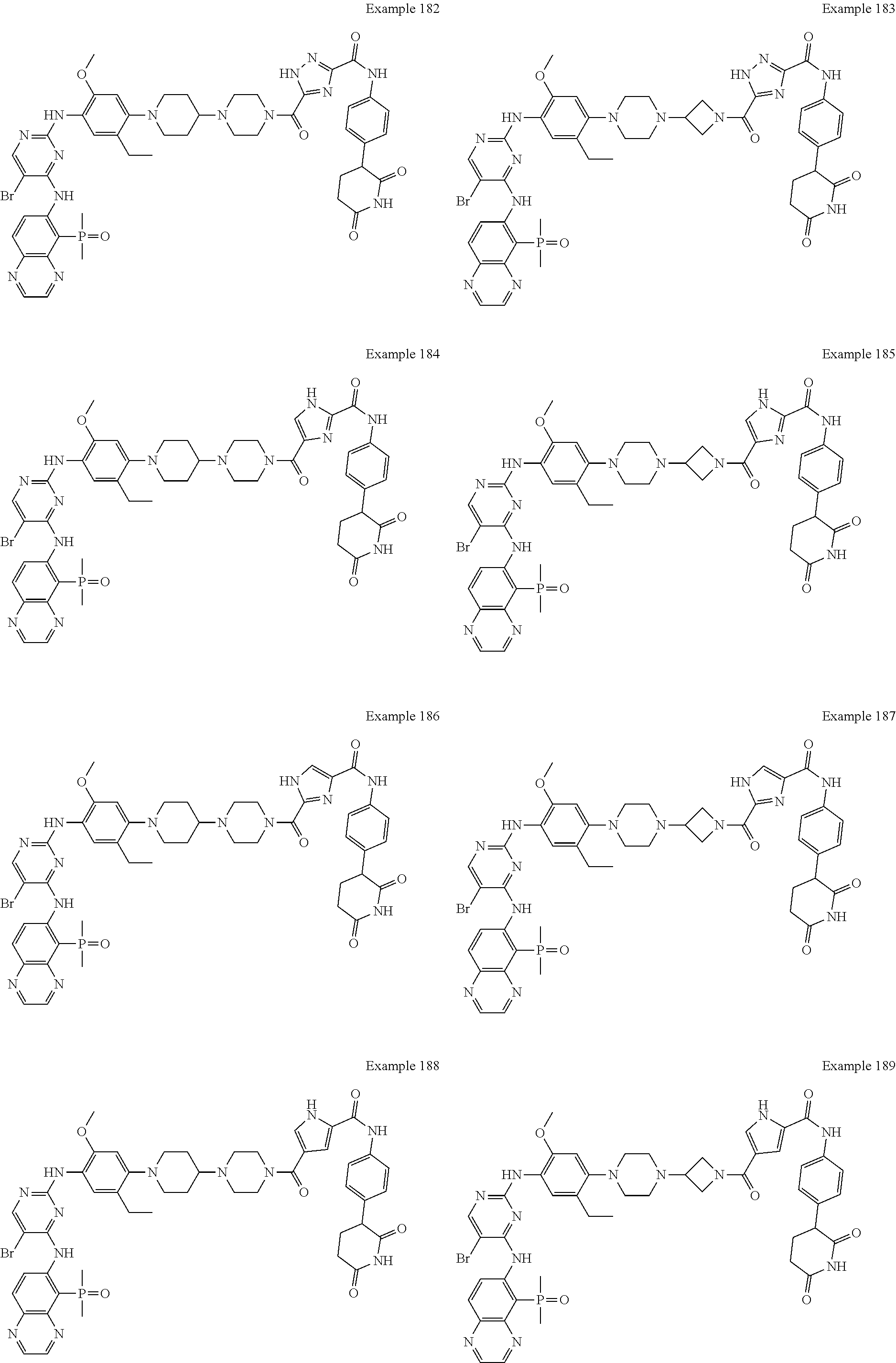

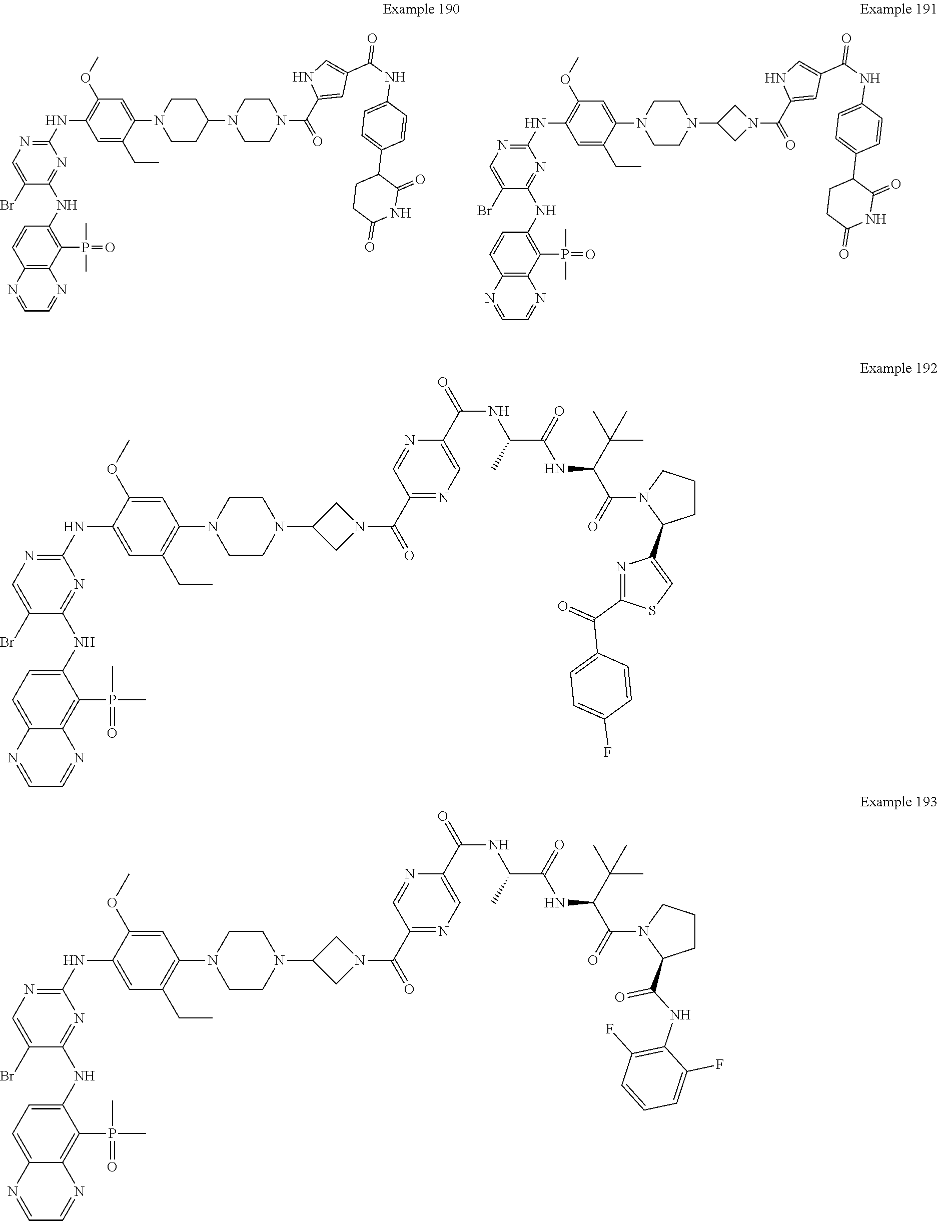

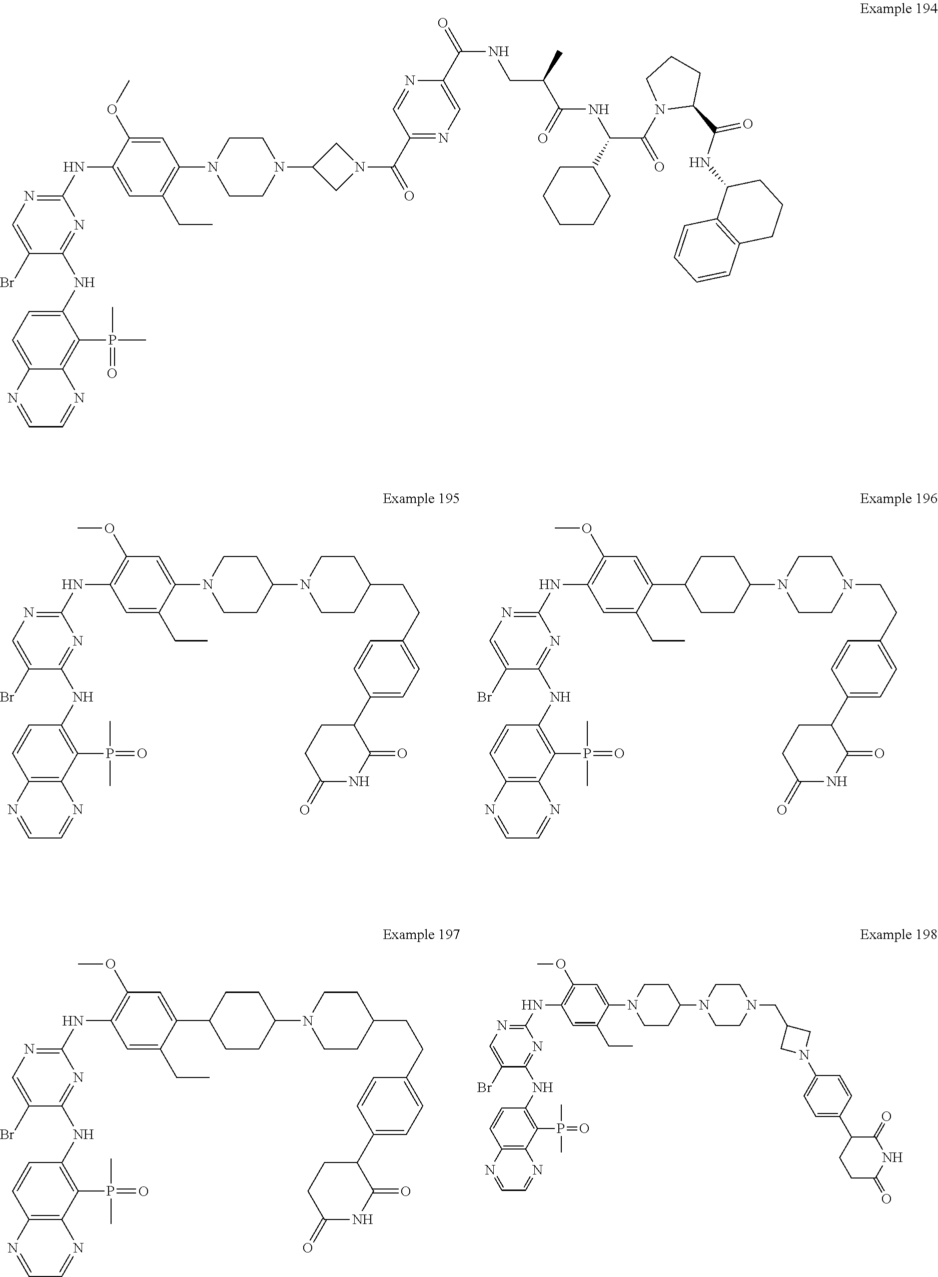

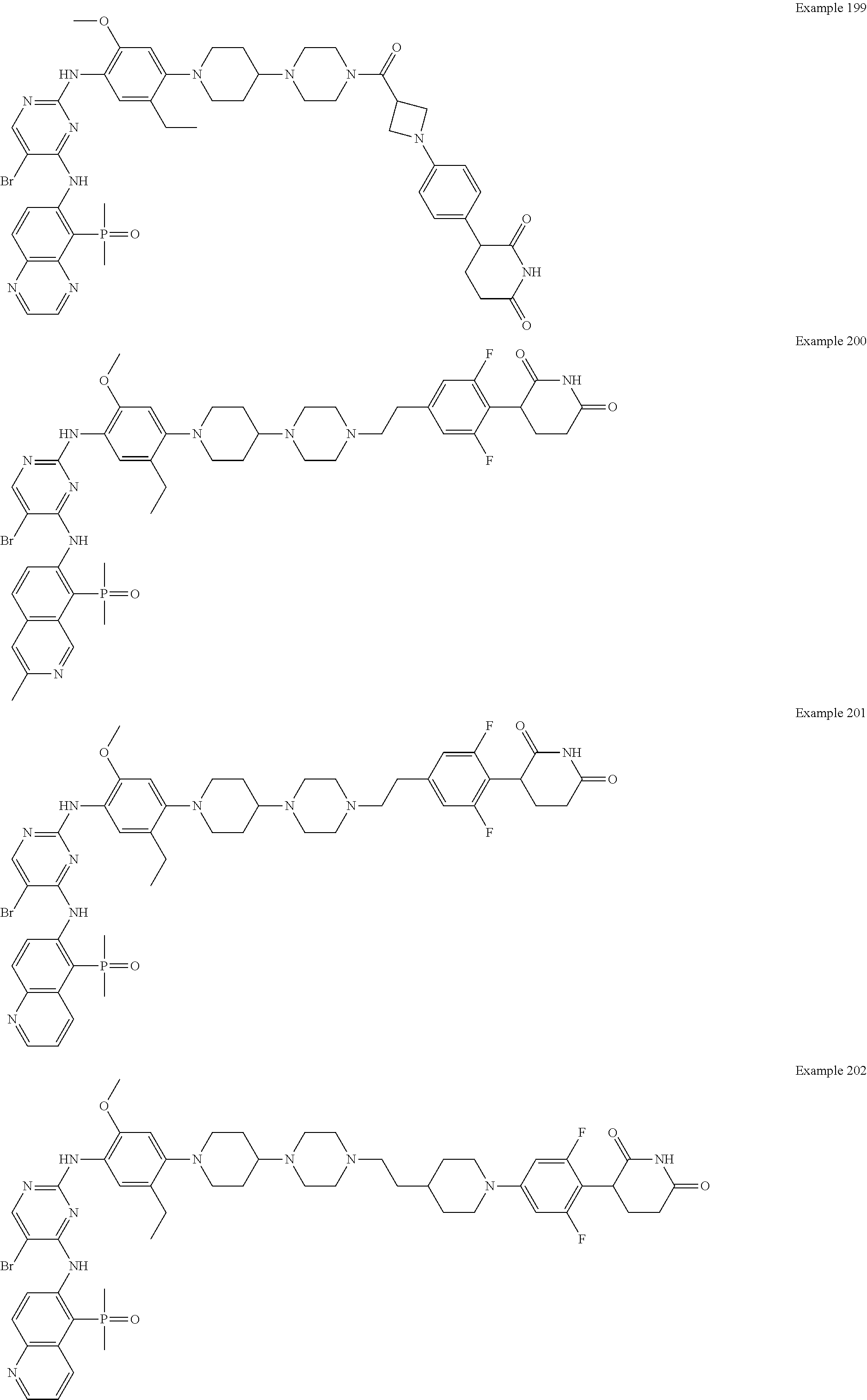

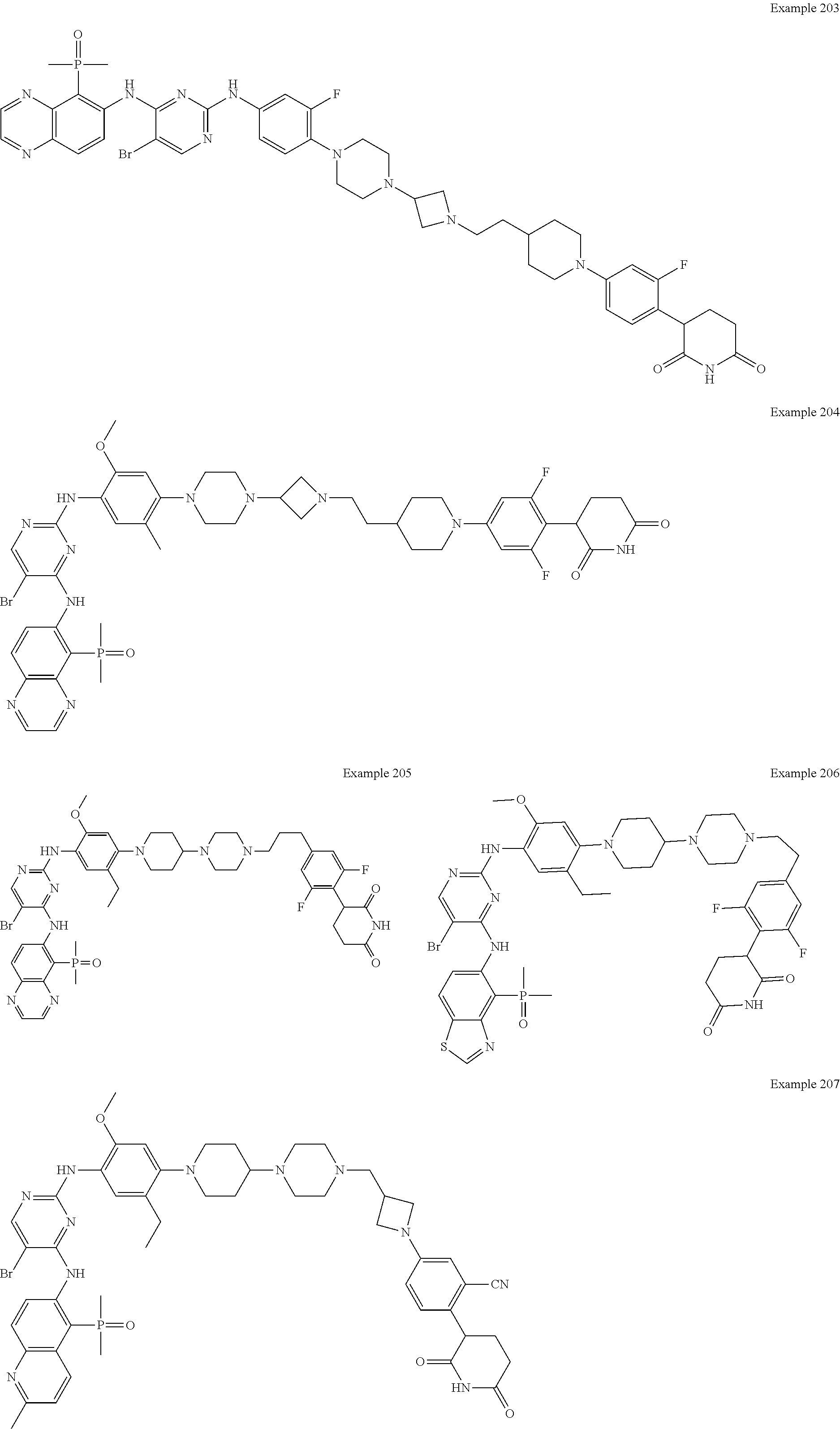

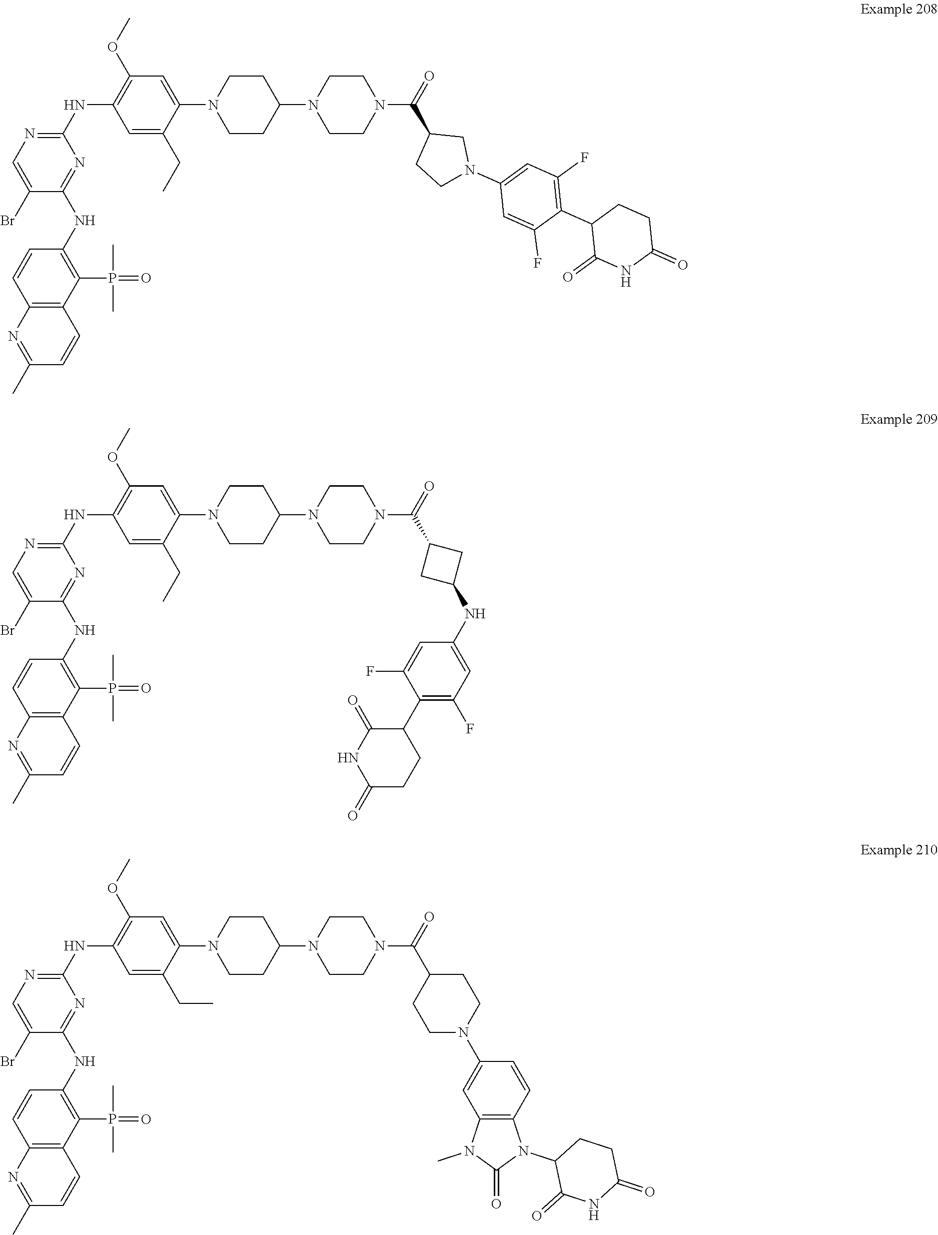 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4cc(F)c(C5CCC(=O)NC5=O)c(F)c4)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)[C@H]3C[C@H](Nc4cc(F)c(C5CCC(=O)NC5=O)c(F)c4)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CCN(c4ccc5c(c4)n(C)c(=O)n5C4CCC(=O)NC4=O)CC3)CC2)CC1]

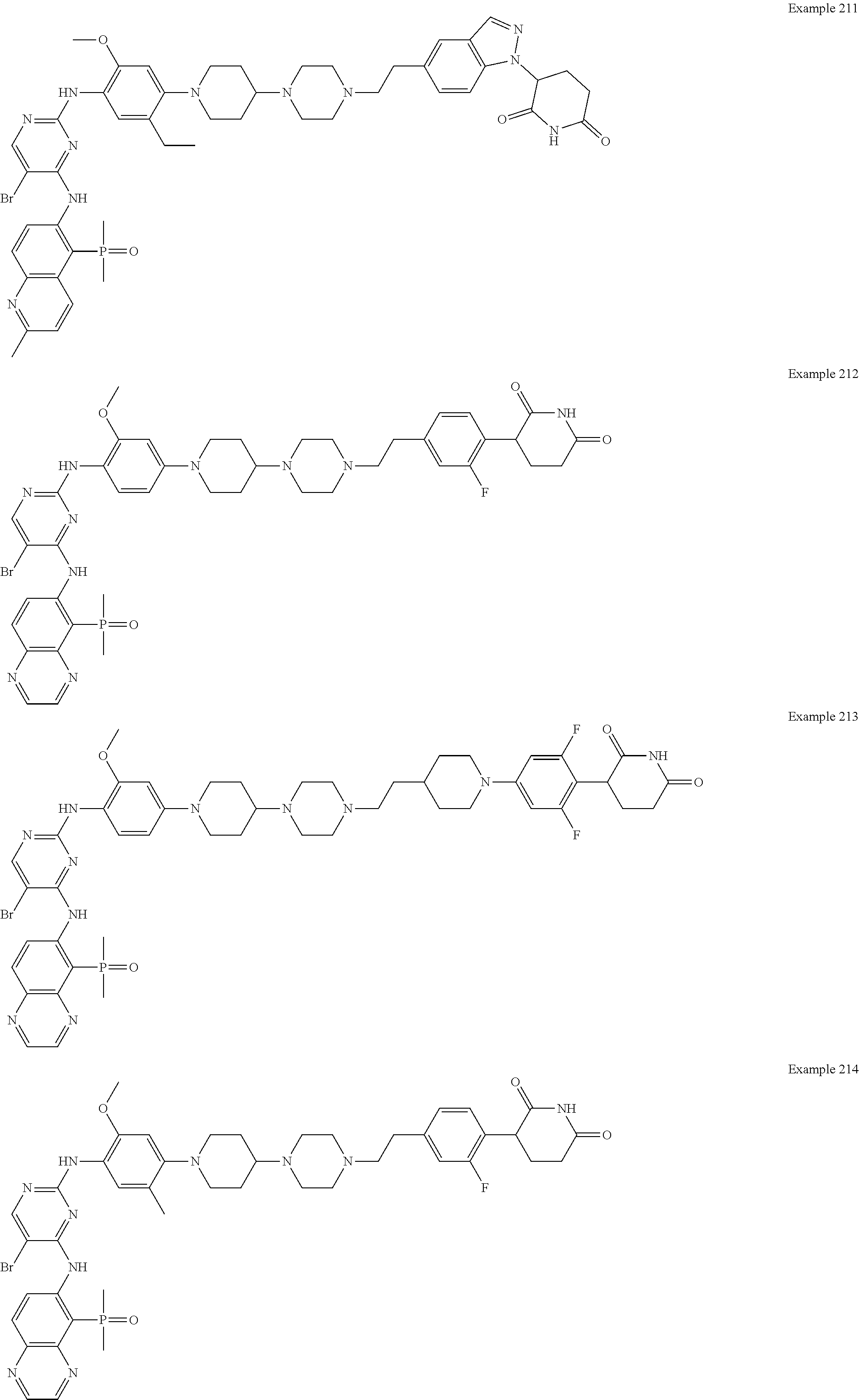 [SMILES: CCc1cc(Nc2ncc(Br)c(Nc3ccc4nc(C)ccc4c3P(C)(C)=O)n2)c(OC)cc1N1CCC(N2CCN(CCc3ccc4c(cnn4C4CCC(=O)NC4=O)c3)CC2)CC1 ; COc1cc(N2CCC(N3CCN(CCc4ccc(C5CCC(=O)NC5=O)c(F)c4)CC3)CC2)ccc1Nc1ncc(Br)c(Nc2ccc3nccnc3c2P(C)(C)=O)n1 ; COc1cc(N2CCC(N3CCN(CCC4CCN(c5cc(F)c(C6CCC(=O)NC6=O)c(F)c5)CC4)CC3)CC2)ccc1Nc1ncc(Br)c(Nc2ccc3nccnc3c2P(C)(C)=O)n1 ; COc1cc(N2CCC(N3CCN(CCc4ccc(C5CCC(=O)NC5=O)c(F)c4)CC3)CC2)c(C)cc1Nc1ncc(Br)c(Nc2ccc3nccnc3c2P(C)(C)=O)n1]

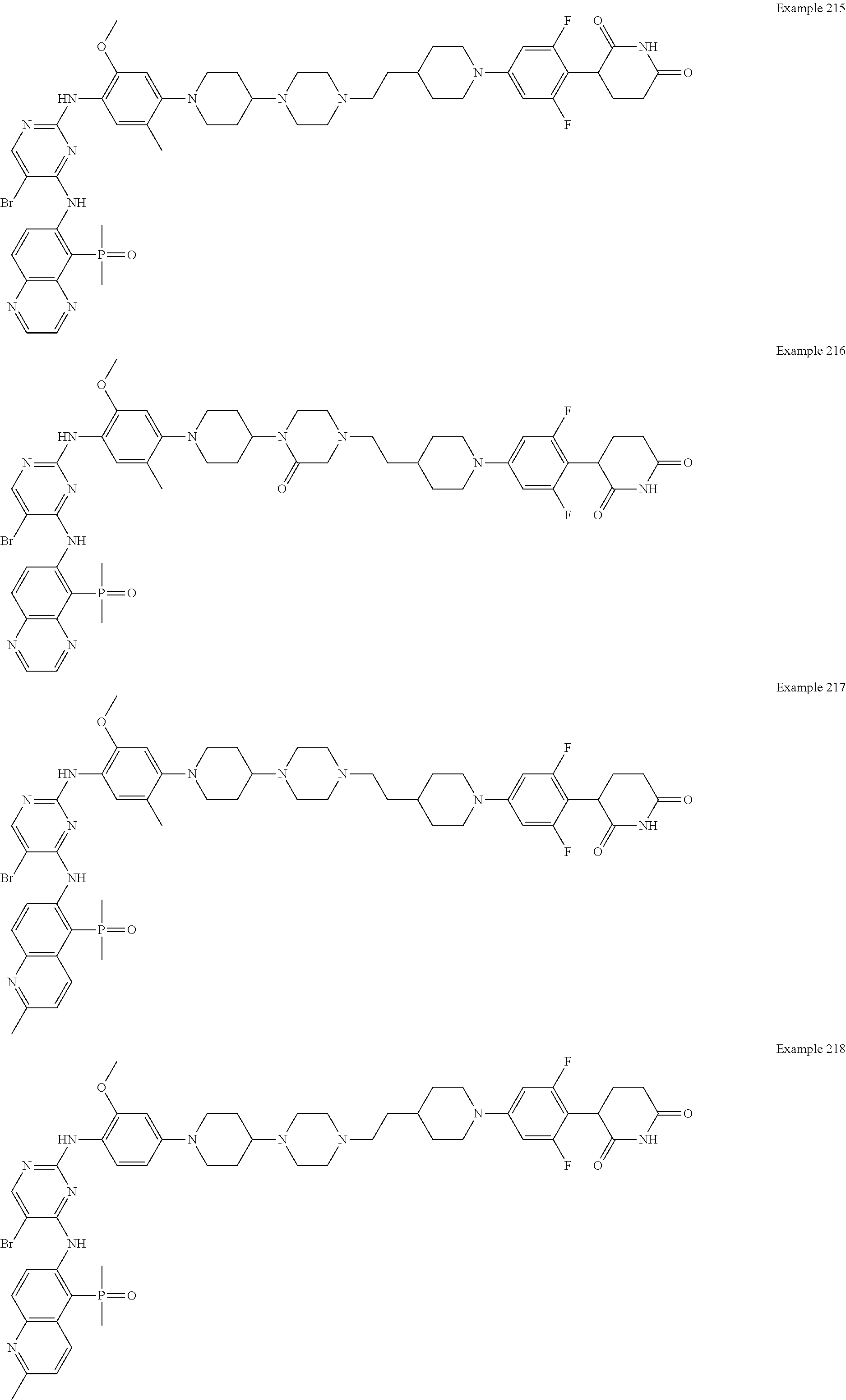 [SMILES: COc1cc(N2CCC(N3CCN(CCC4CCN(c5cc(F)c(C6CCC(=O)NC6=O)c(F)c5)CC4)CC3)CC2)c(C)cc1Nc1ncc(Br)c(Nc2ccc3nccnc3c2P(C)(C)=O)n1 ; COc1cc(N2CCC(N3CCN(CCC4CCN(c5cc(F)c(C6CCC(=O)NC6=O)c(F)c5)CC4)CC3=O)CC2)c(C)cc1Nc1ncc(Br)c(Nc2ccc3nccnc3c2P(C)(C)=O)n1 ; COc1cc(N2CCC(N3CCN(CCC4CCN(c5cc(F)c(C6CCC(=O)NC6=O)c(F)c5)CC4)CC3)CC2)c(C)cc1Nc1ncc(Br)c(Nc2ccc3nc(C)ccc3c2P(C)(C)=O)n1 ; COc1cc(N2CCC(N3CCN(CCC4CCN(c5cc(F)c(C6CCC(=O)NC6=O)c(F)c5)CC4)CC3)CC2)ccc1Nc1ncc(Br)c(Nc2ccc3nc(C)ccc3c2P(C)(C)=O)n1]

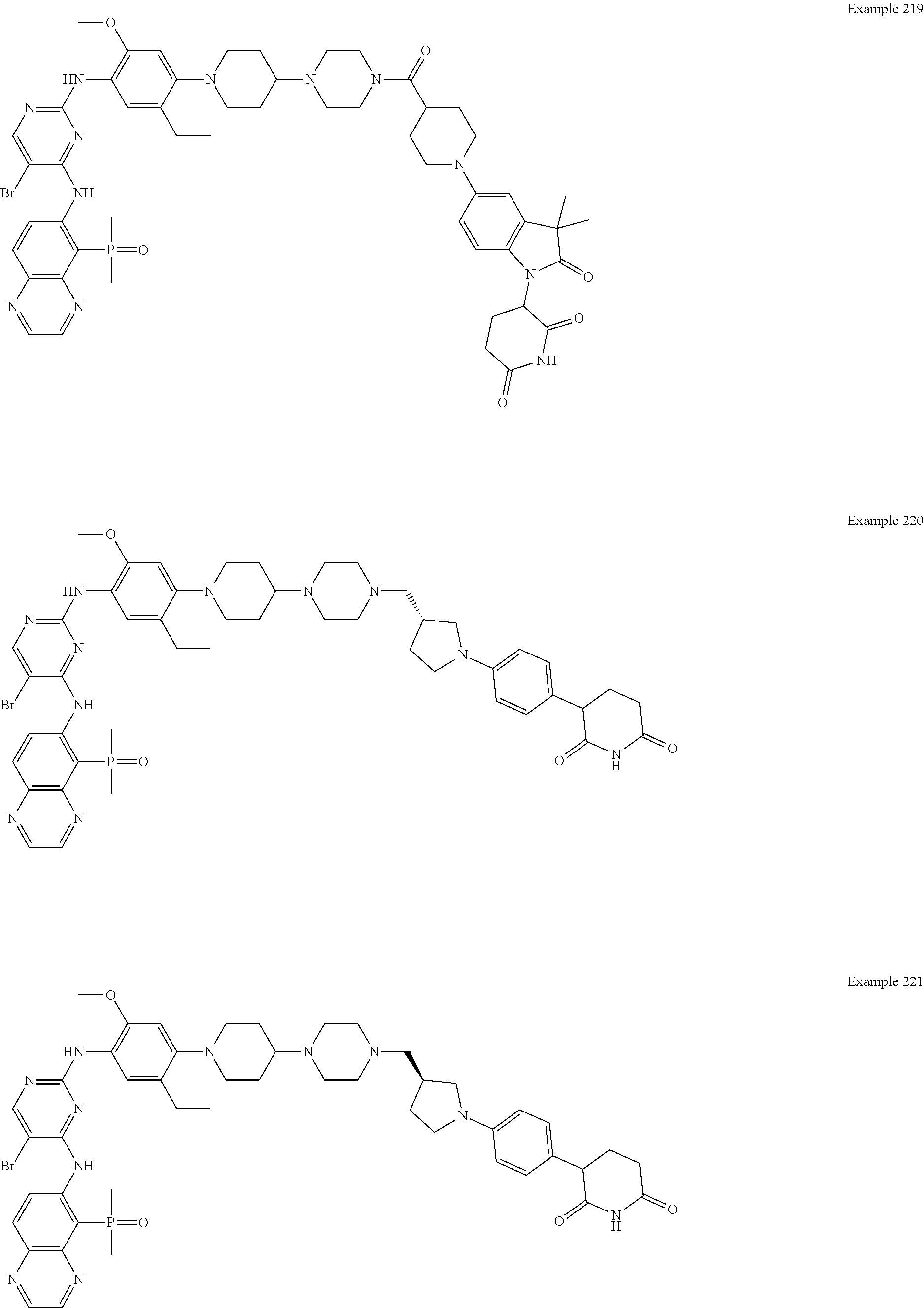 [SMILES: CCc1cc(Nc2ncc(Br)c(Nc3ccc4nccnc4c3P(C)(C)=O)n2)c(OC)cc1N1CCC(N2CCN(C(=O)C3CCN(c4ccc5c(c4)C(C)(C)C(=O)N5C4CCC(=O)NC4=O)CC3)CC2)CC1 ; CCc1cc(Nc2ncc(Br)c(Nc3ccc4nccnc4c3P(C)(C)=O)n2)c(OC)cc1N1CCC(N2CCN(C[C@H]3CCN(c4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(C[C@@H]3CCN(c4ccc(C5CCC(=O)NC5=O)cc4)C3)CC2)CC1]

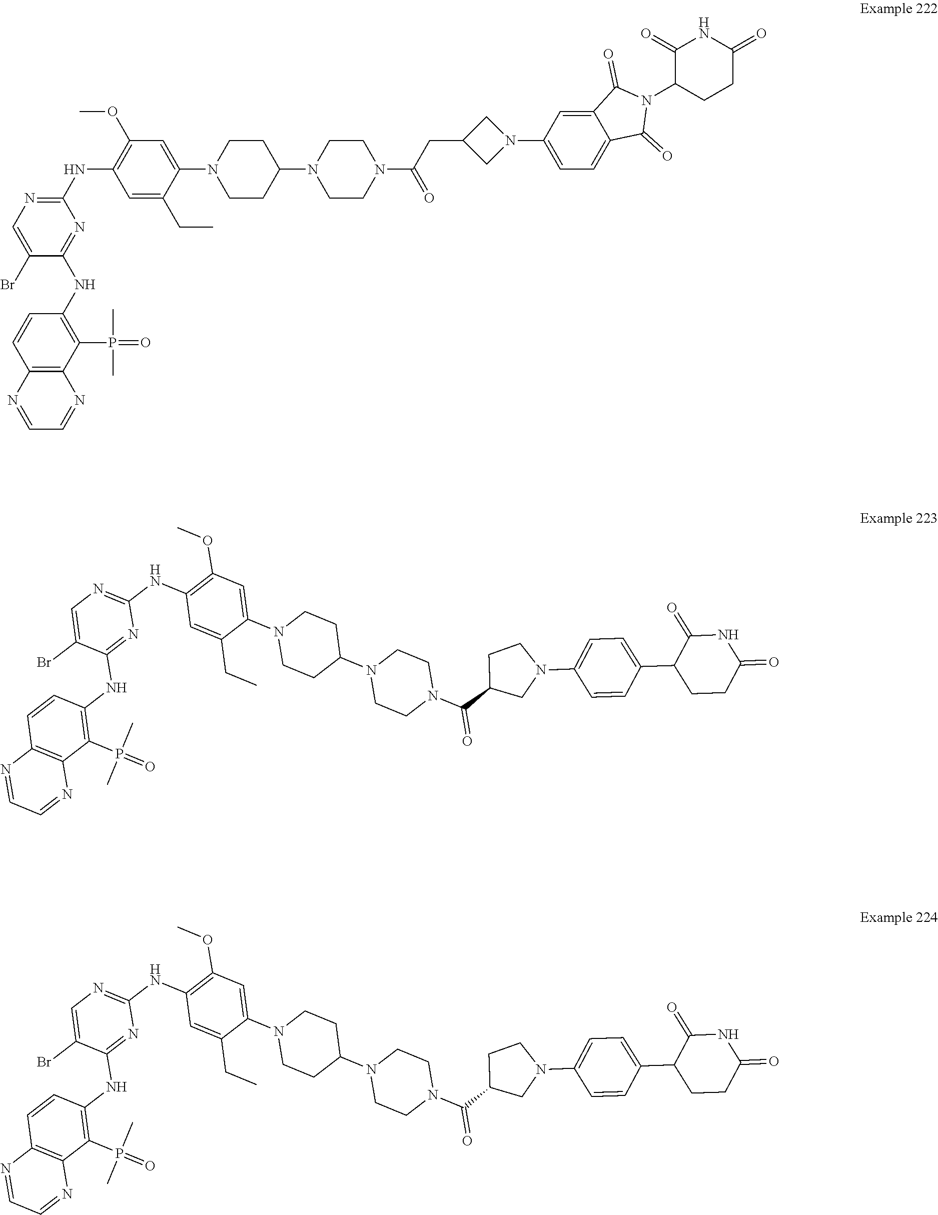 [SMILES: CCc1cc(Nc2ncc(Br)c(Nc3ccc4nccnc4c3P(C)(C)=O)n2)c(OC)cc1N1CCC(N2CCN(C(=O)CC3CN(c4ccc5c(c4)C(=O)N(C4CCC(=O)NC4=O)C5=O)C3)CC2)CC1 ; CCc1cc(Nc2ncc(Br)c(Nc3ccc4nccnc4c3P(C)(C)=O)n2)c(OC)cc1N1CCC(N2CCN(C(=O)[C@H]3CCN(c4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(C(=O)[C@@H]3CCN(c4ccc(C5CCC(=O)NC5=O)cc4)C3)CC2)CC1]

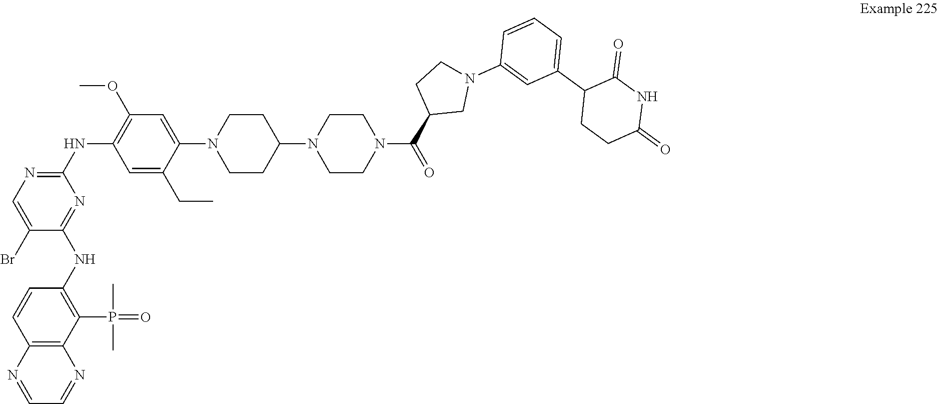 [SMILES: CCc1cc(Nc2ncc(Br)c(Nc3ccc4nccnc4c3P(C)(C)=O)n2)c(OC)cc1N1CCC(N2CCN(C(=O)[C@H]3CCN(c4cccc(C5CCC(=O)NC5=O)c4)C3)CC2)CC1]

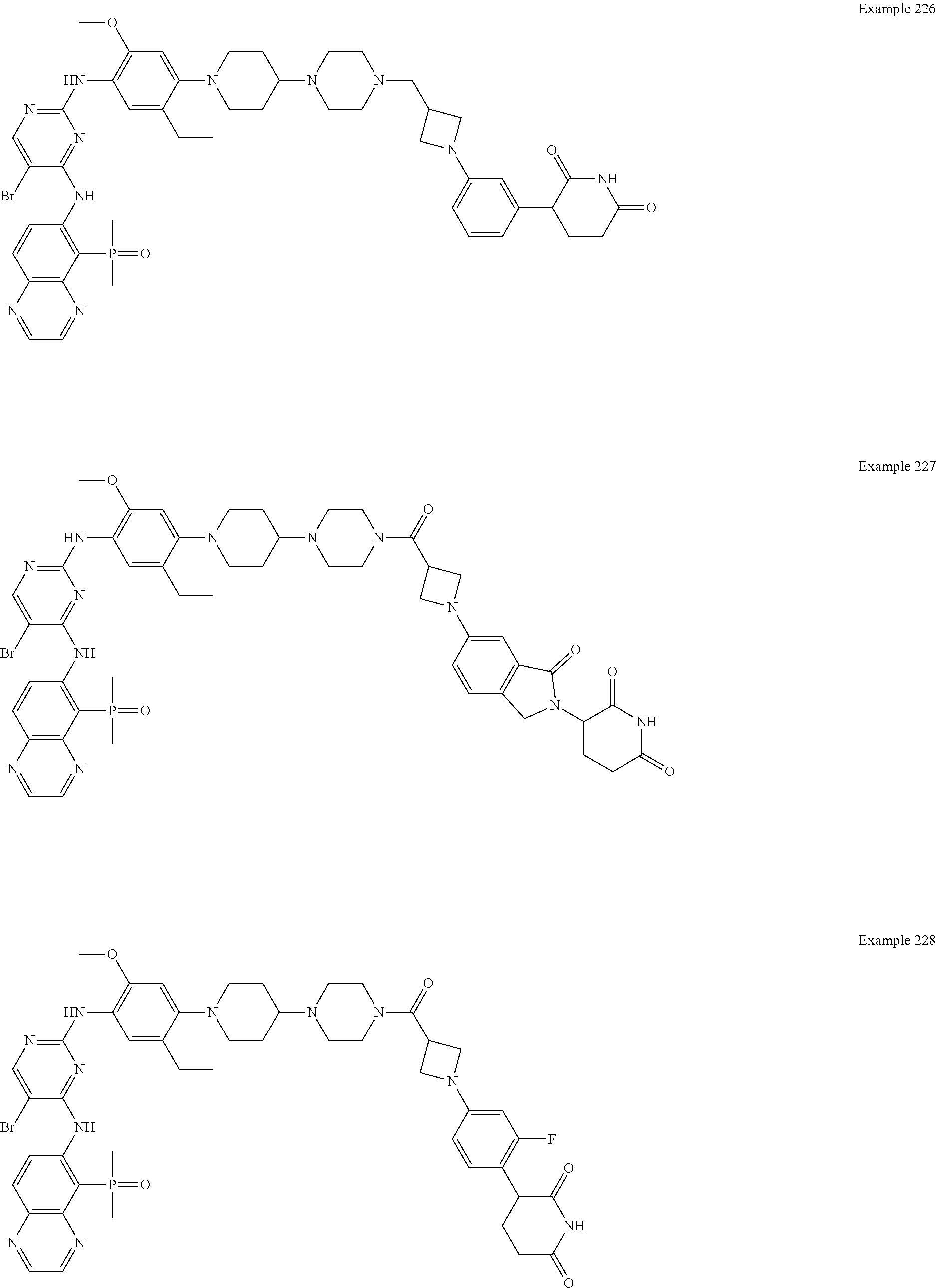 [SMILES: CCc1cc(Nc2ncc(Br)c(Nc3ccc4nccnc4c3P(C)(C)=O)n2)c(OC)cc1N1CCC(N2CCN(CC3CN(c4cccc(C5CCC(=O)NC5=O)c4)C3)CC2)CC1 ; CCc1cc(Nc2ncc(Br)c(Nc3ccc4nccnc4c3P(C)(C)=O)n2)c(OC)cc1N1CCC(N2CCN(C(=O)C3CN(c4ccc5c(c4)C(=O)N(C4CCC(=O)NC4=O)C5)C3)CC2)CC1 ; CCc1cc(Nc2ncc(Br)c(Nc3ccc4nccnc4c3P(C)(C)=O)n2)c(OC)cc1N1CCC(N2CCN(C(=O)C3CN(c4ccc(C5CCC(=O)NC5=O)c(F)c4)C3)CC2)CC1]

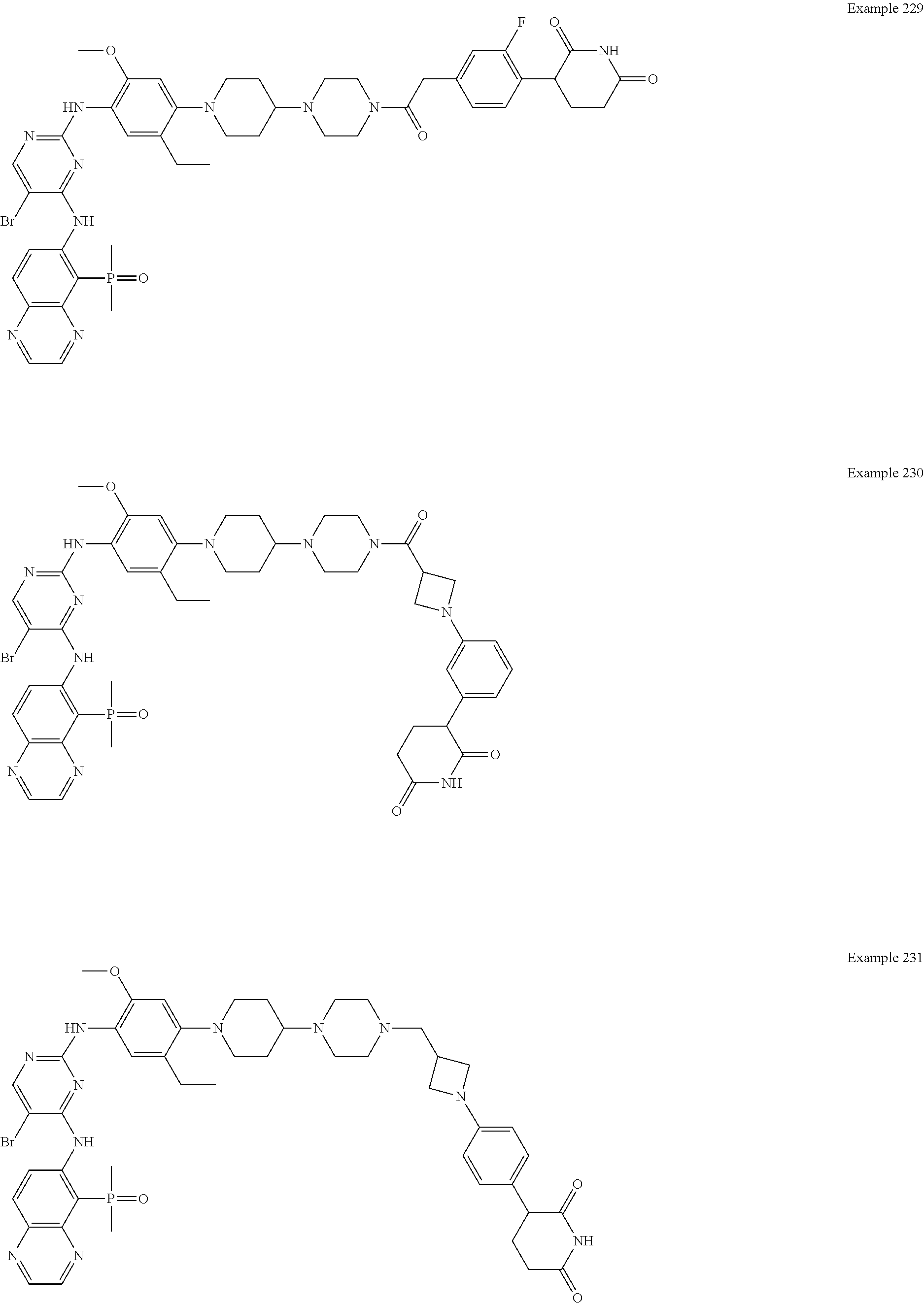 [SMILES: CCc1cc(Nc2ncc(Br)c(Nc3ccc4nccnc4c3P(C)(C)=O)n2)c(OC)cc1N1CCC(N2CCN(C(=O)Cc3ccc(C4CCC(=O)NC4=O)c(F)c3)CC2)CC1 ; CCc1cc(Nc2ncc(Br)c(Nc3ccc4nccnc4c3P(C)(C)=O)n2)c(OC)cc1N1CCC(N2CCN(C(=O)C3CN(c4cccc(C5CCC(=O)NC5=O)c4)C3)CC2)CC1 ; CCc1cc(Nc2ncc(Br)c(Nc3ccc4nccnc4c3P(C)(C)=O)n2)c(OC)cc1N1CCC(N2CCN(CC3CN(c4ccc(C5CCC(=O)NC5=O)cc4)C3)CC2)CC1]

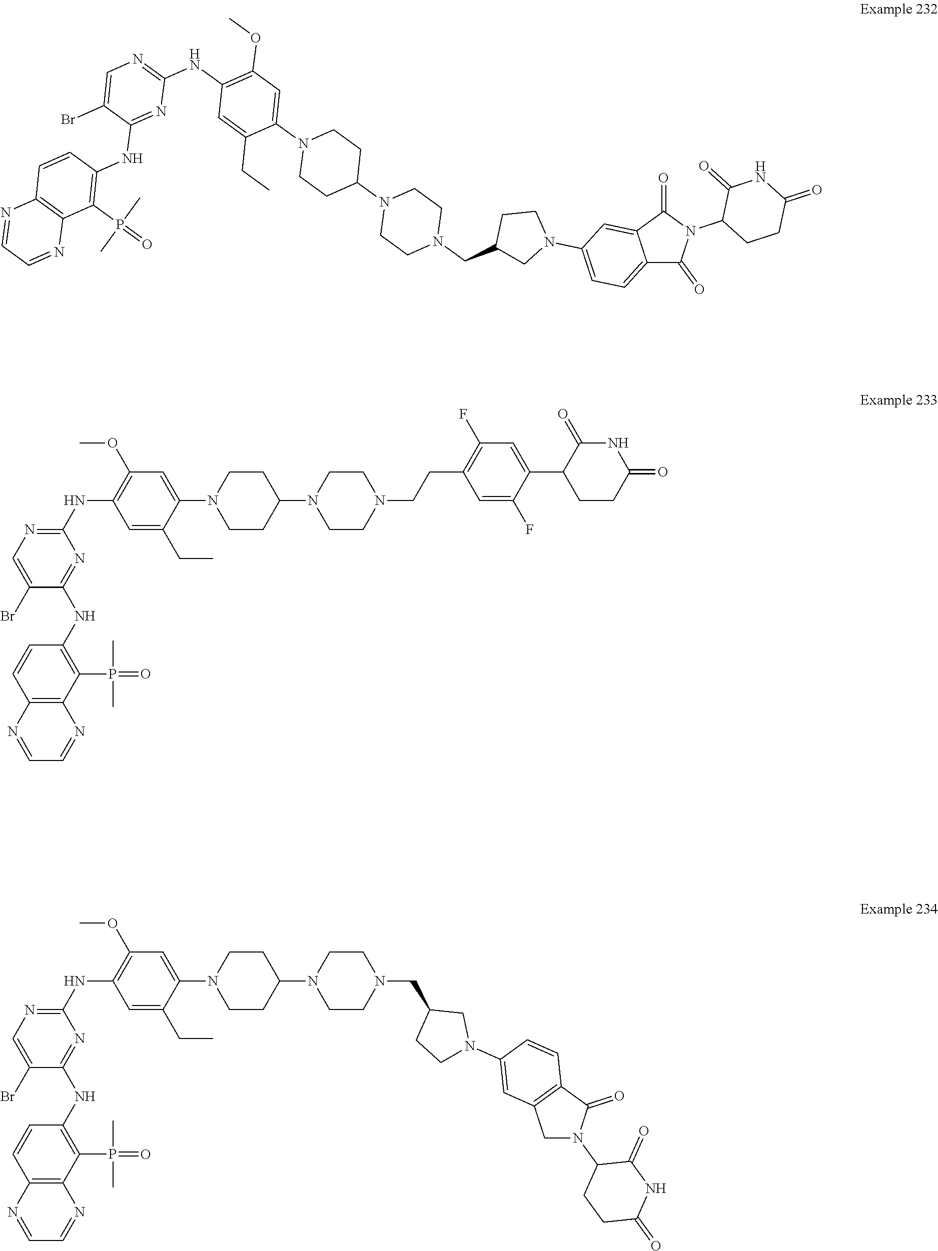

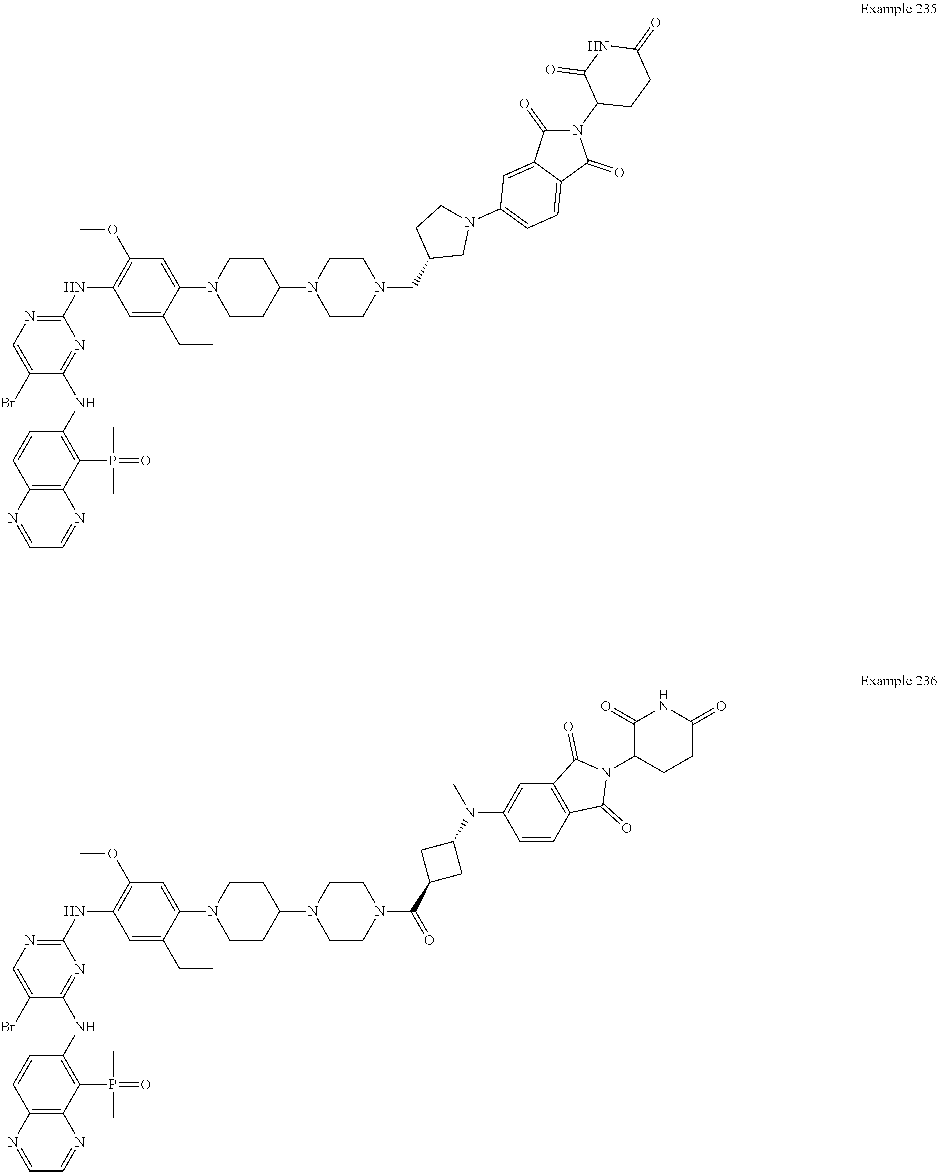 [SMILES: CCc1cc(Nc2ncc(Br)c(Nc3ccc4nccnc4c3P(C)(C)=O)n2)c(OC)cc1N1CCC(N2CCN(C[C@@H]3CCN(c4ccc5c(c4)C(=O)N(C4CCC(=O)NC4=O)C5=O)C3)CC2)CC1 ; CCc1cc(Nc2ncc(Br)c(Nc3ccc4nccnc4c3P(C)(C)=O)n2)c(OC)cc1N1CCC(N2CCN(C(=O)[C@H]3C[C@H](N(C)c4ccc5c(c4)C(=O)N(C4CCC(=O)NC4=O)C5=O)C3)CC2)CC1]

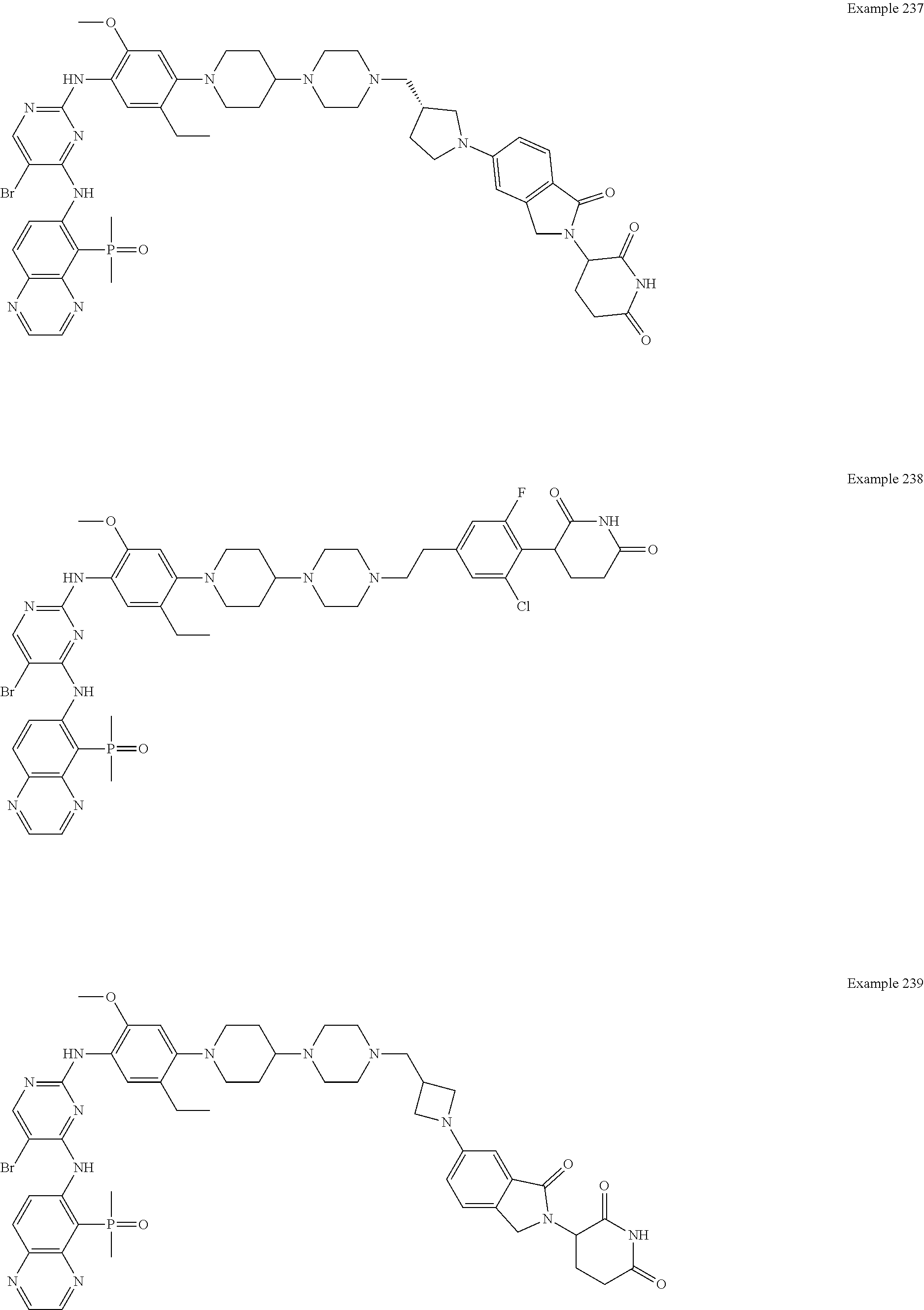 [SMILES: CCc1cc(Nc2ncc(Br)c(Nc3ccc4nccnc4c3P(C)(C)=O)n2)c(OC)cc1N1CCC(N2CCN(C[C@H]3CCN(c4ccc5c(c4)CN(C4CCC(=O)NC4=O)C5=O)C3)CC2)CC1 ; CCc1cc(Nc2ncc(Br)c(Nc3ccc4nccnc4c3P(C)(C)=O)n2)c(OC)cc1N1CCC(N2CCN(CCc3cc(F)c(C4CCC(=O)NC4=O)c(Cl)c3)CC2)CC1 ; CCc1cc(Nc2ncc(Br)c(Nc3ccc4nccnc4c3P(C)(C)=O)n2)c(OC)cc1N1CCC(N2CCN(CC3CN(c4ccc5c(c4)C(=O)N(C4CCC(=O)NC4=O)C5)C3)CC2)CC1]

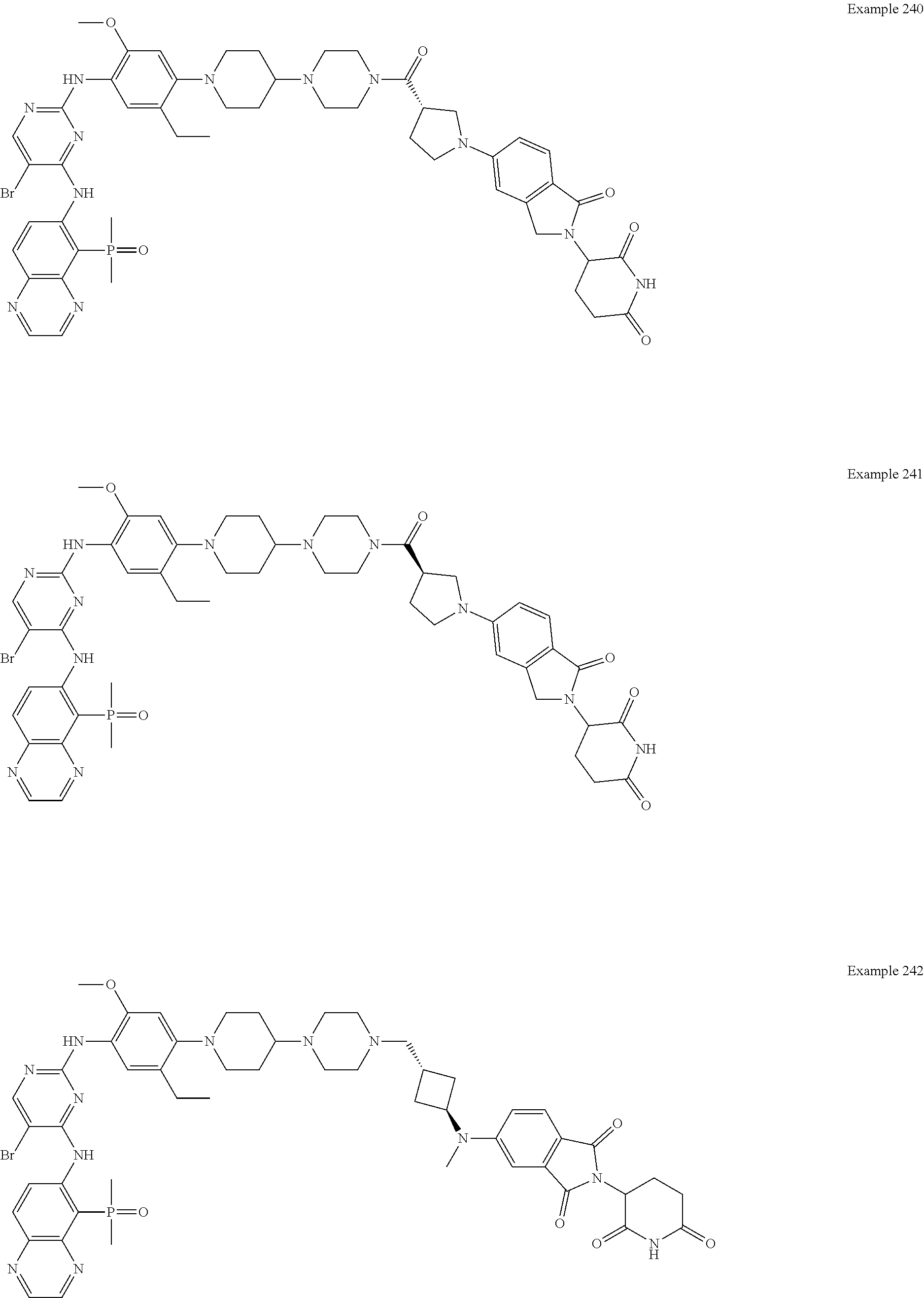 [SMILES: CCc1cc(Nc2ncc(Br)c(Nc3ccc4nccnc4c3P(C)(C)=O)n2)c(OC)cc1N1CCC(N2CCN(C(=O)[C@H]3CCN(c4ccc5c(c4)CN(C4CCC(=O)NC4=O)C5=O)C3)CC2)CC1 ; CCc1cc(Nc2ncc(Br)c(Nc3ccc4nccnc4c3P(C)(C)=O)n2)c(OC)cc1N1CCC(N2CCN(C(=O)[C@@H]3CCN(c4ccc5c(c4)CN(C4CCC(=O)NC4=O)C5=O)C3)CC2)CC1 ; CCc1cc(Nc2ncc(Br)c(Nc3ccc4nccnc4c3P(C)(C)=O)n2)c(OC)cc1N1CCC(N2CCN(C[C@H]3C[C@H](N(C)c4ccc5c(c4)C(=O)N(C4CCC(=O)NC4=O)C5=O)C3)CC2)CC1]

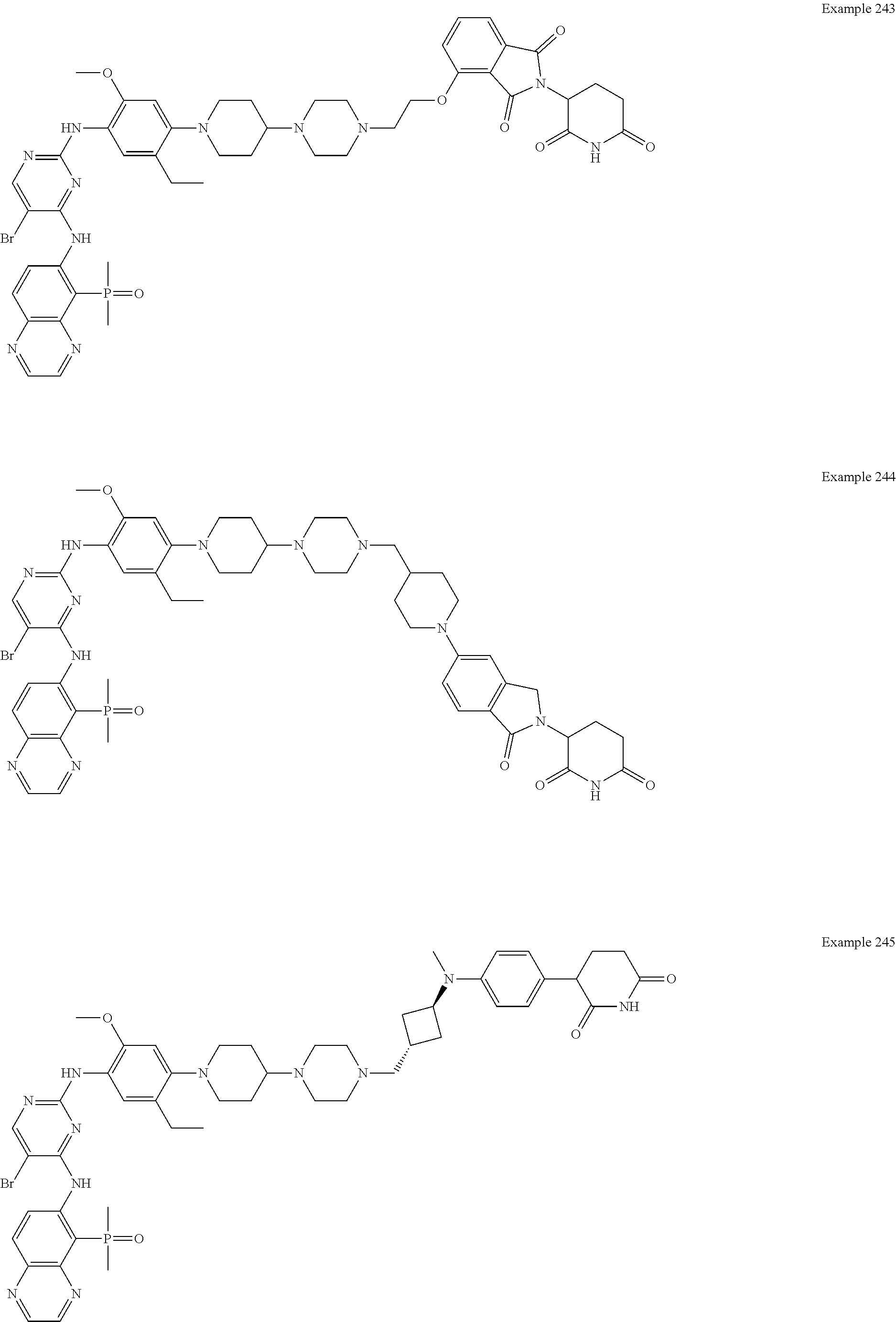 [SMILES: CCc1cc(Nc2ncc(Br)c(Nc3ccc4nccnc4c3P(C)(C)=O)n2)c(OC)cc1N1CCC(N2CCN(CCOc3cccc4c3C(=O)N(C3CCC(=O)NC3=O)C4=O)CC2)CC1 ; CCc1cc(Nc2ncc(Br)c(Nc3ccc4nccnc4c3P(C)(C)=O)n2)c(OC)cc1N1CCC(N2CCN(CC3CCN(c4ccc5c(c4)CN(C4CCC(=O)NC4=O)C5=O)CC3)CC2)CC1 ; CCc1cc(Nc2ncc(Br)c(Nc3ccc4nccnc4c3P(C)(C)=O)n2)c(OC)cc1N1CCC(N2CCN(C[C@H]3C[C@H](N(C)c4ccc(C5CCC(=O)NC5=O)cc4)C3)CC2)CC1]

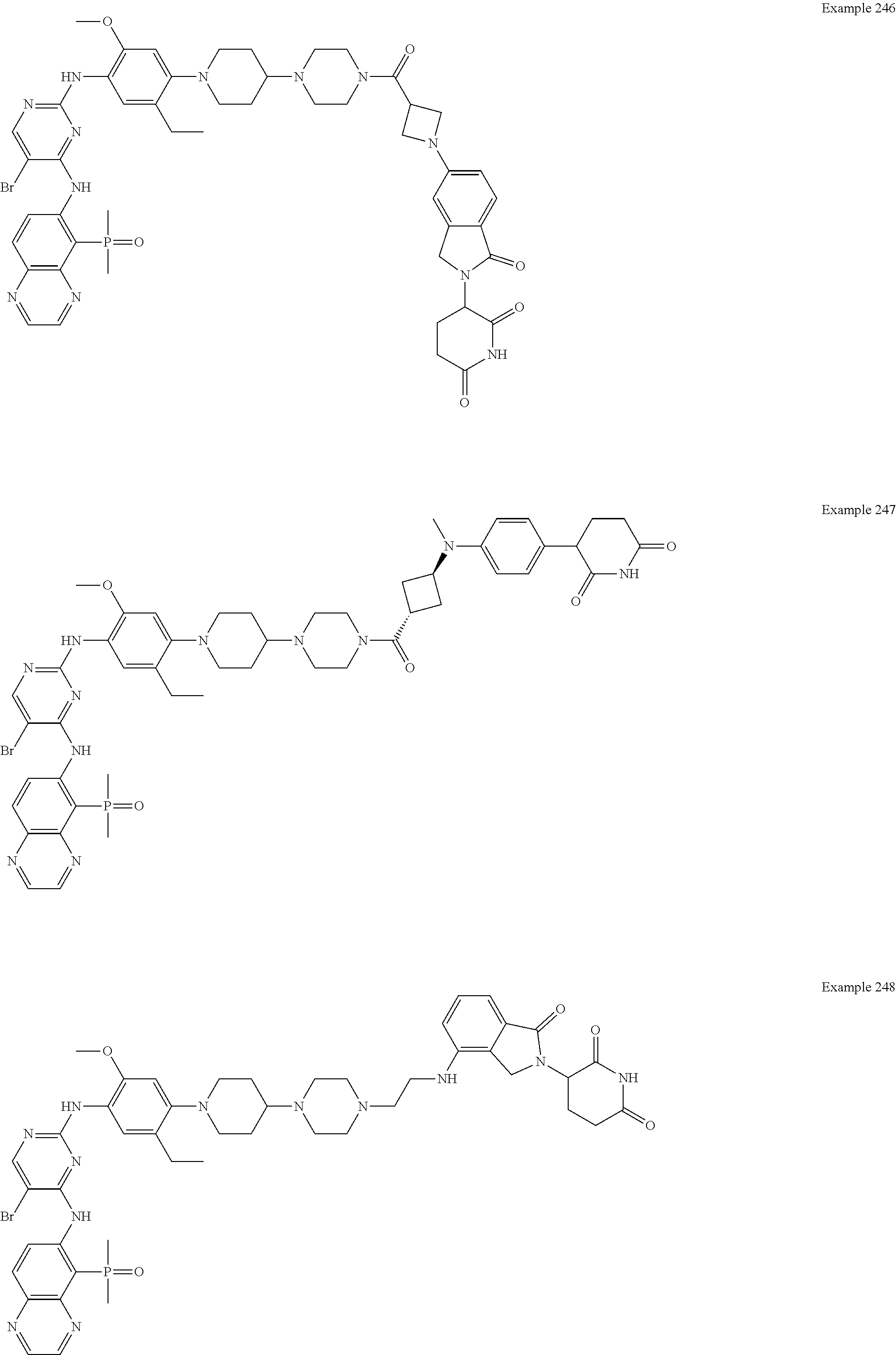 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc5c(c4)CN(C4CCC(=O)NC4=O)C5=O)C3)CC2)CC1 ; CCc1cc(Nc2ncc(Br)c(Nc3ccc4nccnc4c3P(C)(C)=O)n2)c(OC)cc1N1CCC(N2CCN(C(=O)[C@H]3C[C@H](N(C)c4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(CCNc3cccc4c3CN(C3CCC(=O)NC3=O)C4=O)CC2)CC1]

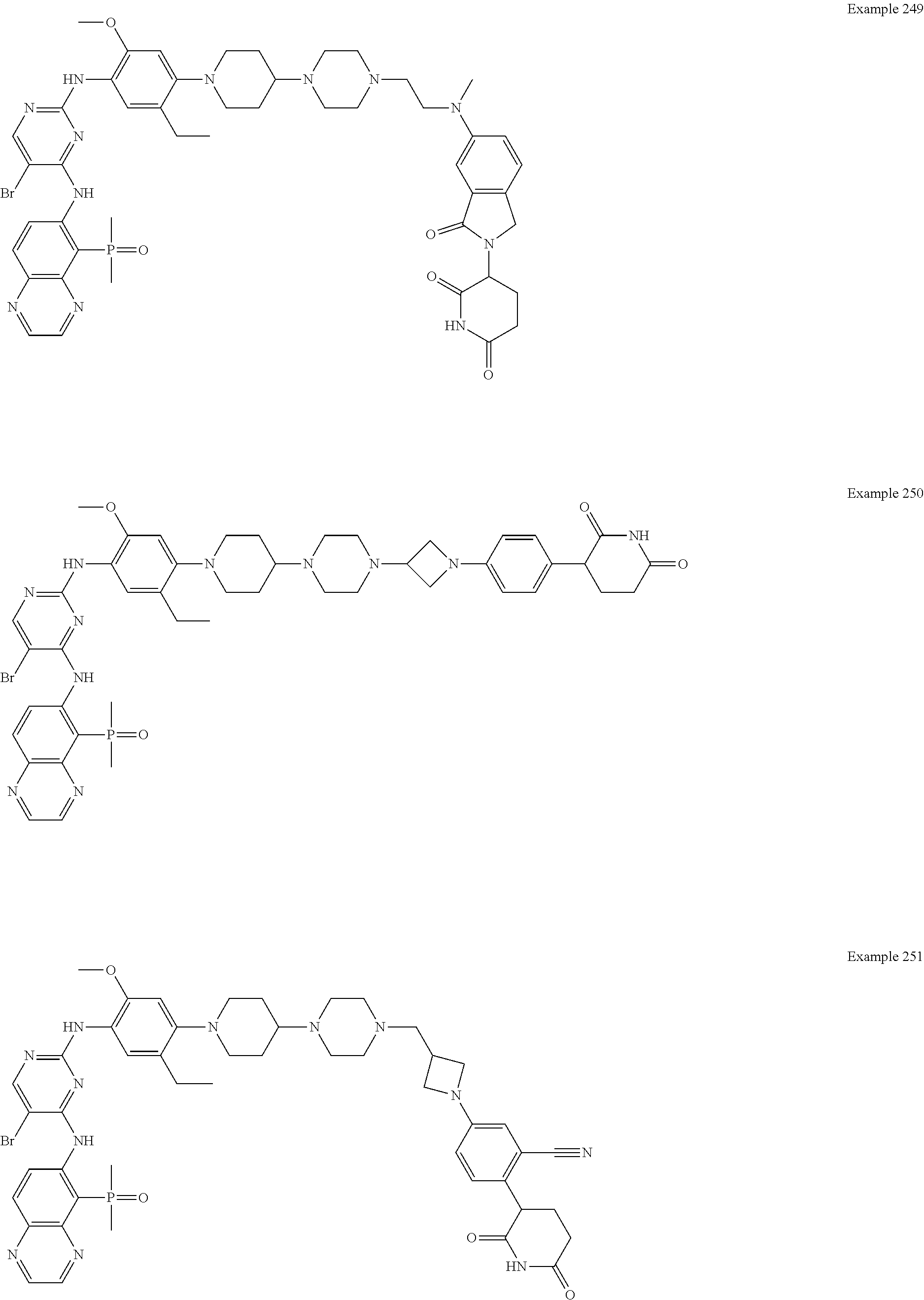 [SMILES: CCc1cc(Nc2ncc(Br)c(Nc3ccc4nccnc4c3P(C)(C)=O)n2)c(OC)cc1N1CCC(N2CCN(CCN(C)c3ccc4c(c3)C(=O)N(C3CCC(=O)NC3=O)C4)CC2)CC1 ; CCc1cc(Nc2ncc(Br)c(Nc3ccc4nccnc4c3P(C)(C)=O)n2)c(OC)cc1N1CCC(N2CCN(C3CN(c4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(CC3CN(c4ccc(C5CCC(=O)NC5=O)c(C#N)c4)C3)CC2)CC1]

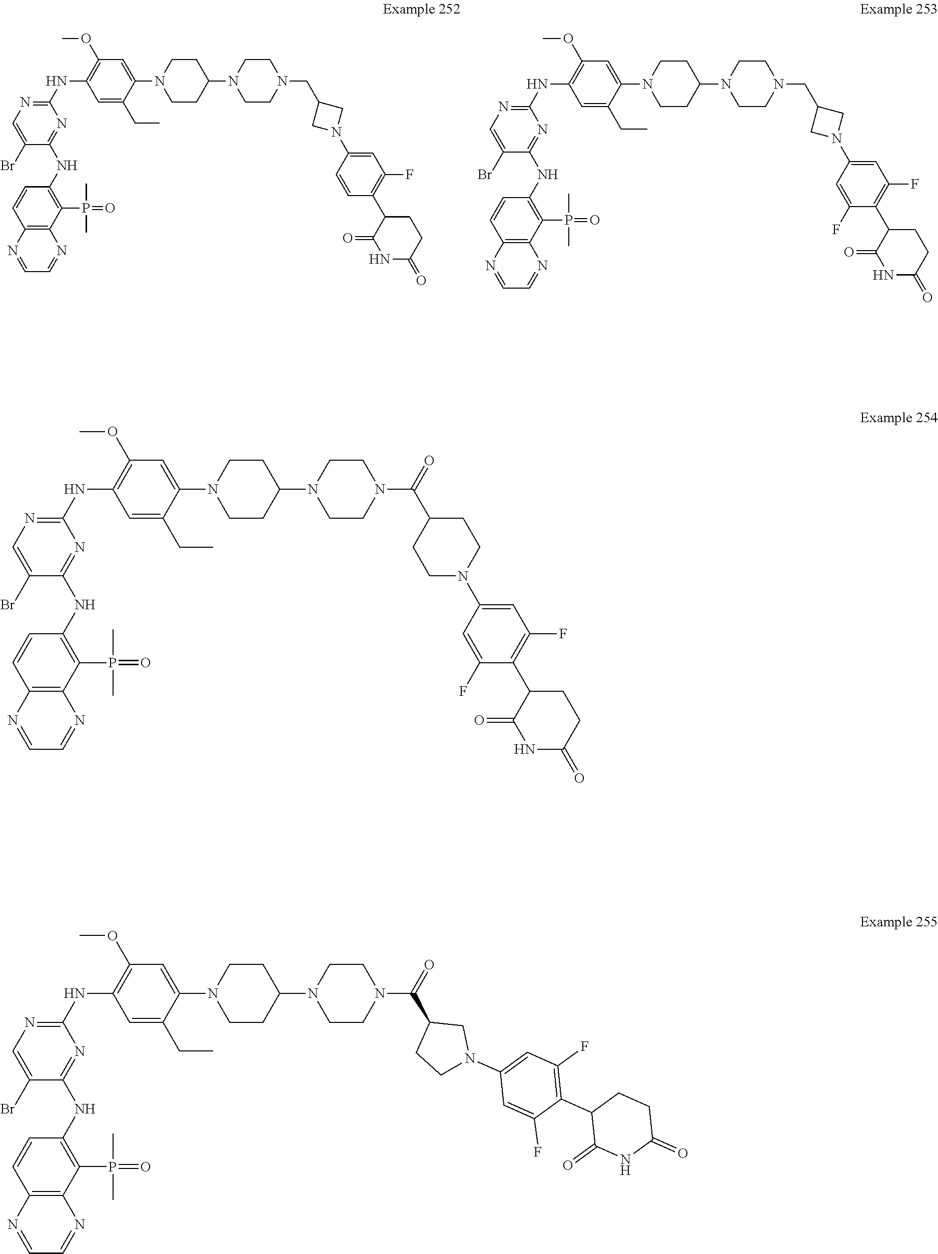 [SMILES: CCc1cc(Nc2ncc(Br)c(Nc3ccc4nccnc4c3P(C)(C)=O)n2)c(OC)cc1N1CCC(N2CCN(CC3CN(c4ccc(C5CCC(=O)NC5=O)c(F)c4)C3)CC2)CC1 ; CCc1cc(Nc2ncc(Br)c(Nc3ccc4nccnc4c3P(C)(C)=O)n2)c(OC)cc1N1CCC(N2CCN(CC3CN(c4cc(F)c(C5CCC(=O)NC5=O)c(F)c4)C3)CC2)CC1 ; CCc1cc(Nc2ncc(Br)c(Nc3ccc4nccnc4c3P(C)(C)=O)n2)c(OC)cc1N1CCC(N2CCN(C(=O)C3CCN(c4cc(F)c(C5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(C(=O)[C@@H]3CCN(c4cc(F)c(C5CCC(=O)NC5=O)c(F)c4)C3)CC2)CC1]

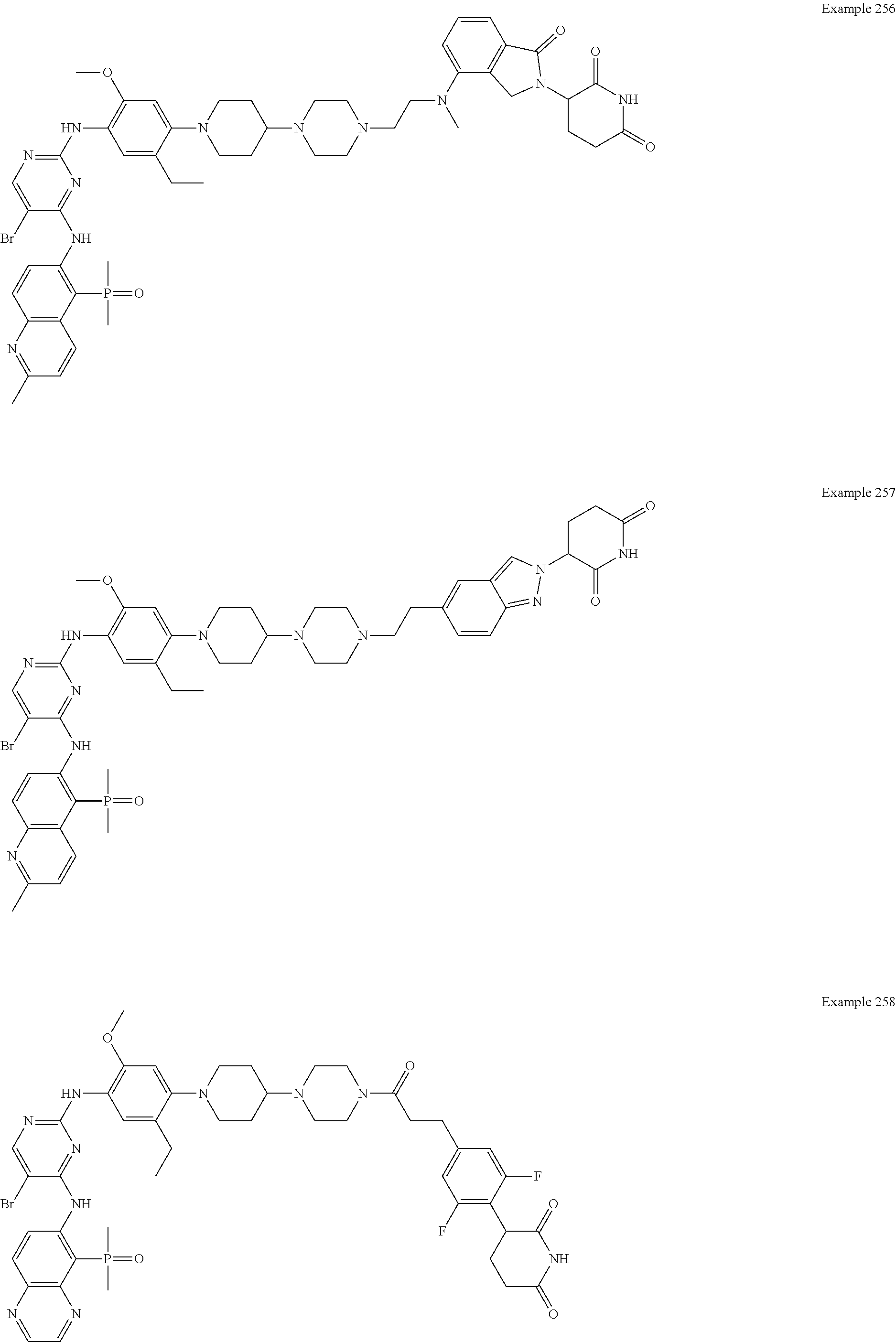 [SMILES: CCc1cc(Nc2ncc(Br)c(Nc3ccc4nc(C)ccc4c3P(C)(C)=O)n2)c(OC)cc1N1CCC(N2CCN(CCN(C)c3cccc4c3CN(C3CCC(=O)NC3=O)C4=O)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4nn(C5CCC(=O)NC5=O)cc4c3)CC2)CC1 ; CCc1cc(Nc2ncc(Br)c(Nc3ccc4nccnc4c3P(C)(C)=O)n2)c(OC)cc1N1CCC(N2CCN(C(=O)CCc3cc(F)c(C4CCC(=O)NC4=O)c(F)c3)CC2)CC1]

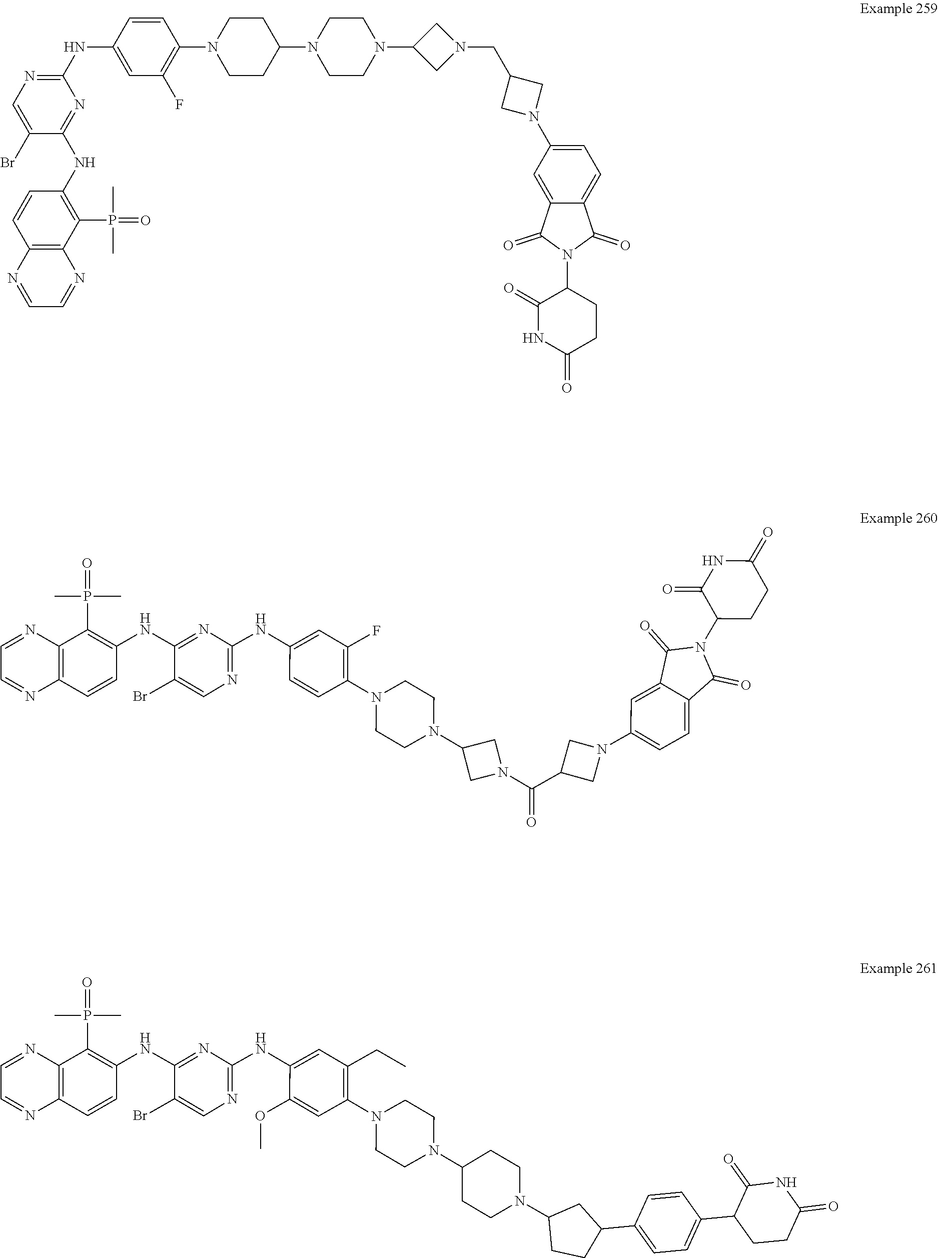 [SMILES: CP(C)(=O)c1c(Nc2nc(Nc3ccc(N4CCC(N5CCN(C6CN(CC7CN(c8ccc9c(c8)C(=O)N(C8CCC(=O)NC8=O)C9=O)C7)C6)CC5)CC4)c(F)c3)ncc2Br)ccc2nccnc12 ; CP(C)(=O)c1c(Nc2nc(Nc3ccc(N4CCN(C5CN(C(=O)C6CN(c7ccc8c(c7)C(=O)N(C7CCC(=O)NC7=O)C8=O)C6)C5)CC4)c(F)c3)ncc2Br)ccc2nccnc12 ; CCc1cc(Nc2ncc(Br)c(Nc3ccc4nccnc4c3P(C)(C)=O)n2)c(OC)cc1N1CCN(C2CCN(C3CCC(c4ccc(C5CCC(=O)NC5=O)cc4)C3)CC2)CC1]

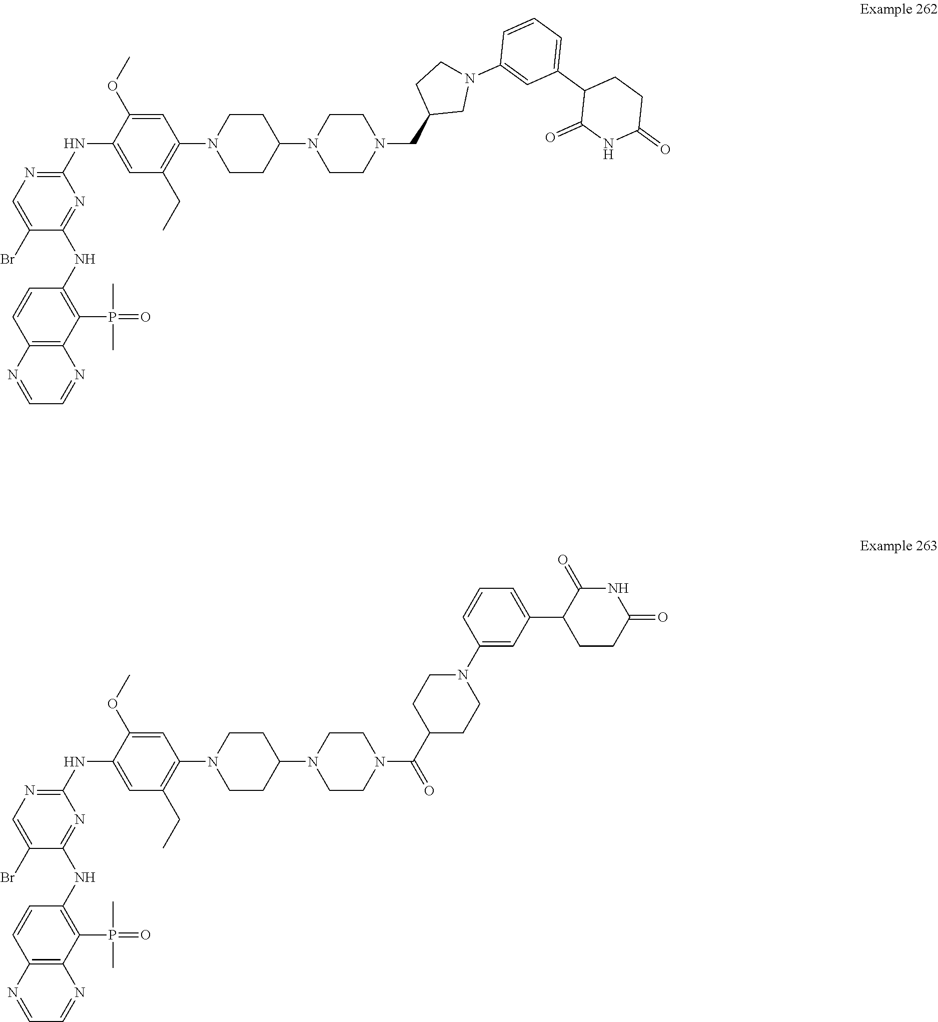 [SMILES: CCc1cc(Nc2ncc(Br)c(Nc3ccc4nccnc4c3P(C)(C)=O)n2)c(OC)cc1N1CCC(N2CCN(C[C@H]3CCN(c4cccc(C5CCC(=O)NC5=O)c4)C3)CC2)CC1 ; CCc1cc(Nc2ncc(Br)c(Nc3ccc4nccnc4c3P(C)(C)=O)n2)c(OC)cc1N1CCC(N2CCN(C(=O)C3CCN(c4cccc(C5CCC(=O)NC5=O)c4)CC3)CC2)CC1]

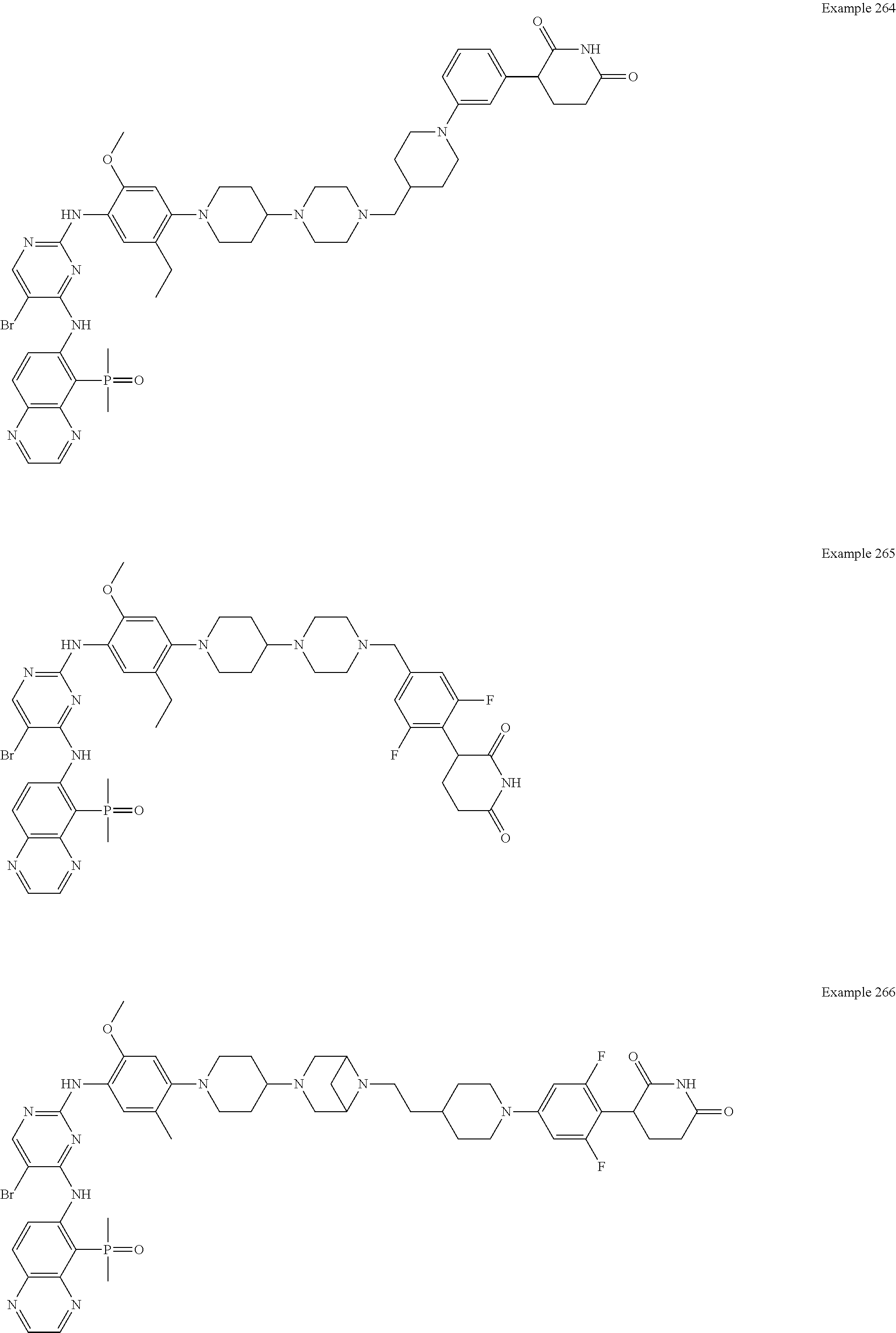 [SMILES: CCc1cc(Nc2ncc(Br)c(Nc3ccc4nccnc4c3P(C)(C)=O)n2)c(OC)cc1N1CCC(N2CCN(CC3CCN(c4cccc(C5CCC(=O)NC5=O)c4)CC3)CC2)CC1 ; CCc1cc(Nc2ncc(Br)c(Nc3ccc4nccnc4c3P(C)(C)=O)n2)c(OC)cc1N1CCC(N2CCN(Cc3cc(F)c(C4CCC(=O)NC4=O)c(F)c3)CC2)CC1 ; COc1cc(N2CCC(N3CC4CC(C3)N4CCC3CCN(c4cc(F)c(C5CCC(=O)NC5=O)c(F)c4)CC3)CC2)c(C)cc1Nc1ncc(Br)c(Nc2ccc3nccnc3c2P(C)(C)=O)n1]

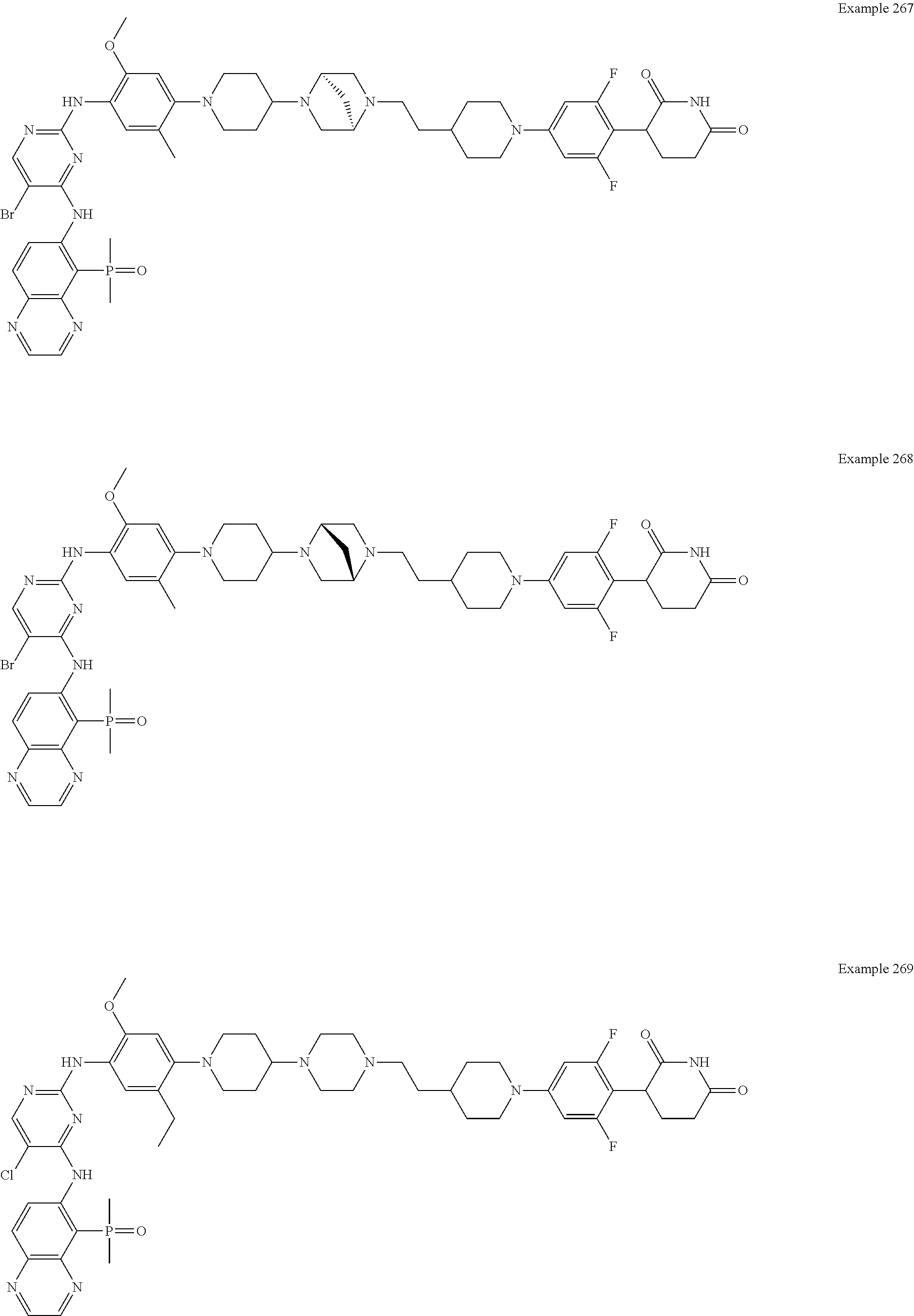 [SMILES: COc1cc(N2CCC(N3C[C@H]4C[C@@H]3CN4CCC3CCN(c4cc(F)c(C5CCC(=O)NC5=O)c(F)c4)CC3)CC2)c(C)cc1Nc1ncc(Br)c(Nc2ccc3nccnc3c2P(C)(C)=O)n1 ; COc1cc(N2CCC(N3C[C@@H]4C[C@H]3CN4CCC3CCN(c4cc(F)c(C5CCC(=O)NC5=O)c(F)c4)CC3)CC2)c(C)cc1Nc1ncc(Br)c(Nc2ccc3nccnc3c2P(C)(C)=O)n1 ; CCc1cc(Nc2ncc(Cl)c(Nc3ccc4nccnc4c3P(C)(C)=O)n2)c(OC)cc1N1CCC(N2CCN(CCC3CCN(c4cc(F)c(C5CCC(=O)NC5=O)c(F)c4)CC3)CC2)CC1]

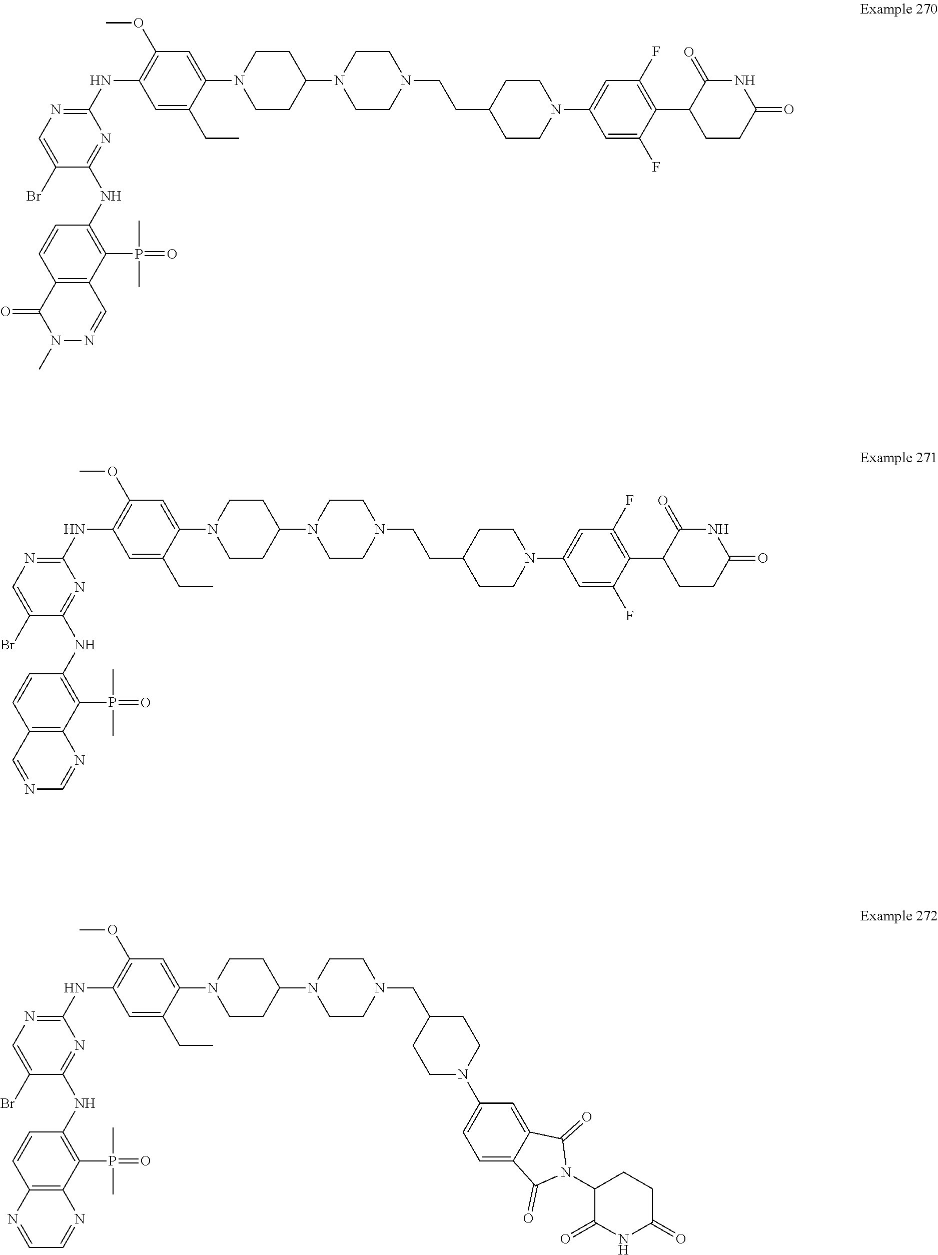 [SMILES: CCc1cc(Nc2ncc(Br)c(Nc3ccc4c(=O)n(C)ncc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4cncnc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(CC3CCN(c4ccc5c(c4)C(=O)N(C4CCC(=O)NC4=O)C5=O)CC3)CC2)CC1]

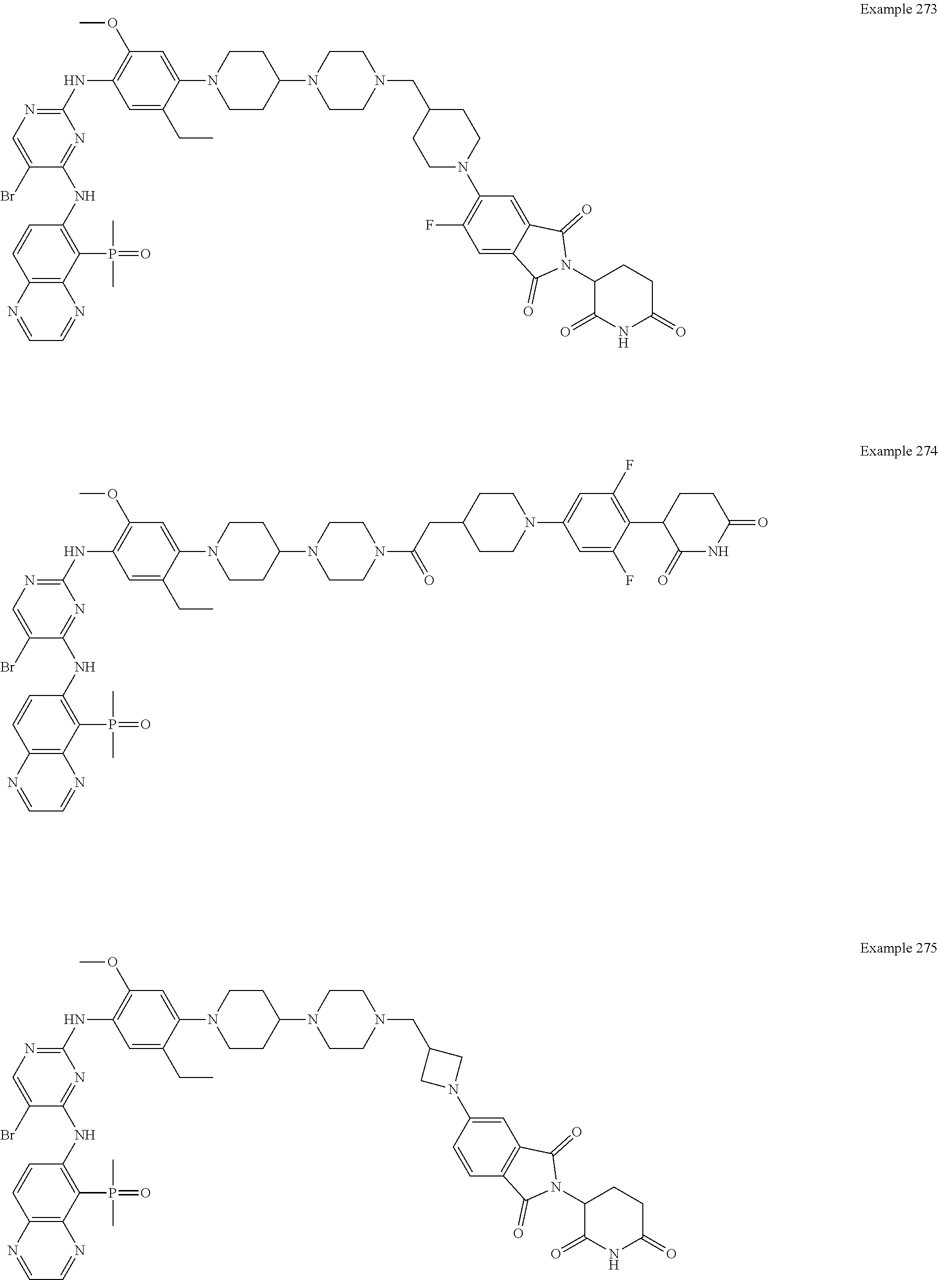 [SMILES: CCc1cc(Nc2ncc(Br)c(Nc3ccc4nccnc4c3P(C)(C)=O)n2)c(OC)cc1N1CCC(N2CCN(CC3CCN(c4cc5c(cc4F)C(=O)N(C4CCC(=O)NC4=O)C5=O)CC3)CC2)CC1 ; CCc1cc(Nc2ncc(Br)c(Nc3ccc4nccnc4c3P(C)(C)=O)n2)c(OC)cc1N1CCC(N2CCN(C(=O)CC3CCN(c4cc(F)c(C5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(CC3CN(c4ccc5c(c4)C(=O)N(C4CCC(=O)NC4=O)C5=O)C3)CC2)CC1]

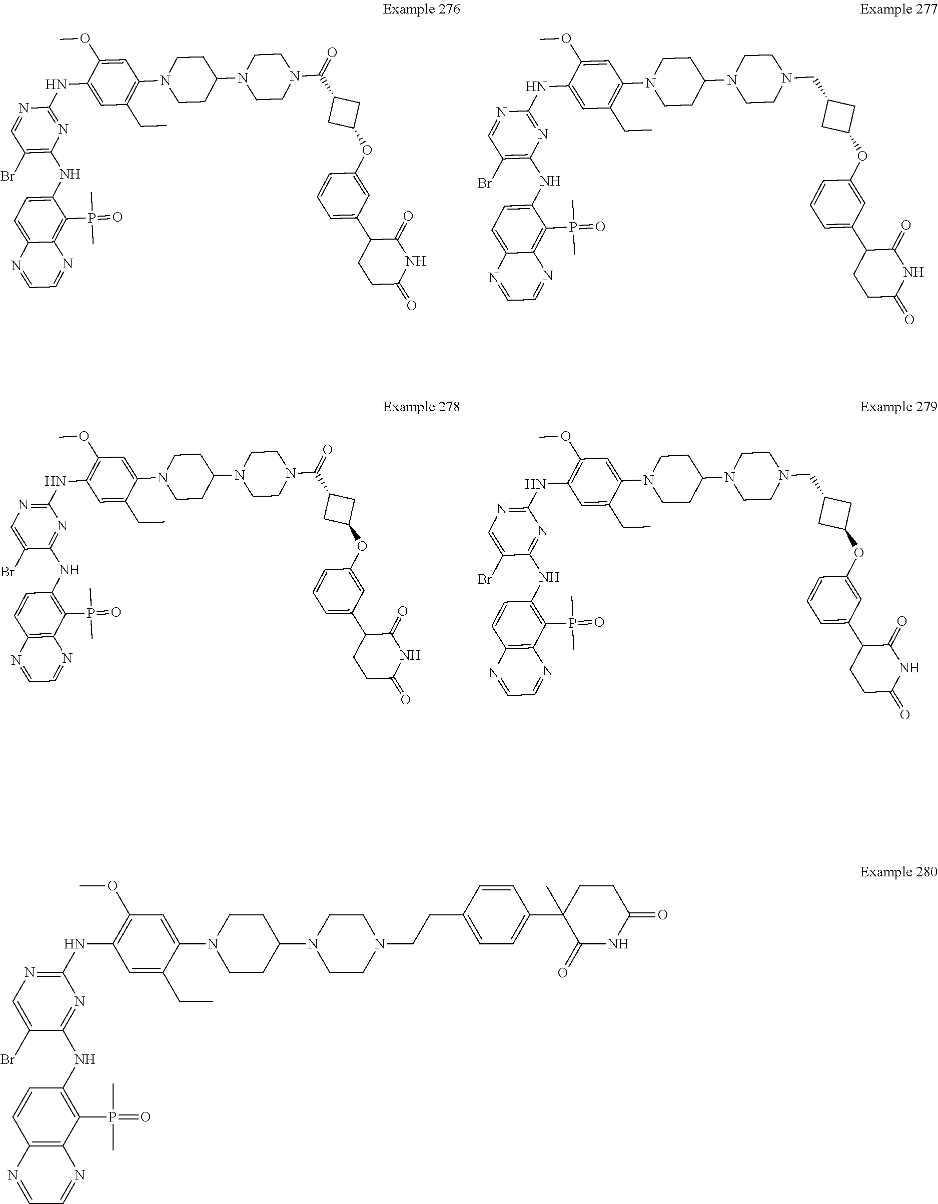

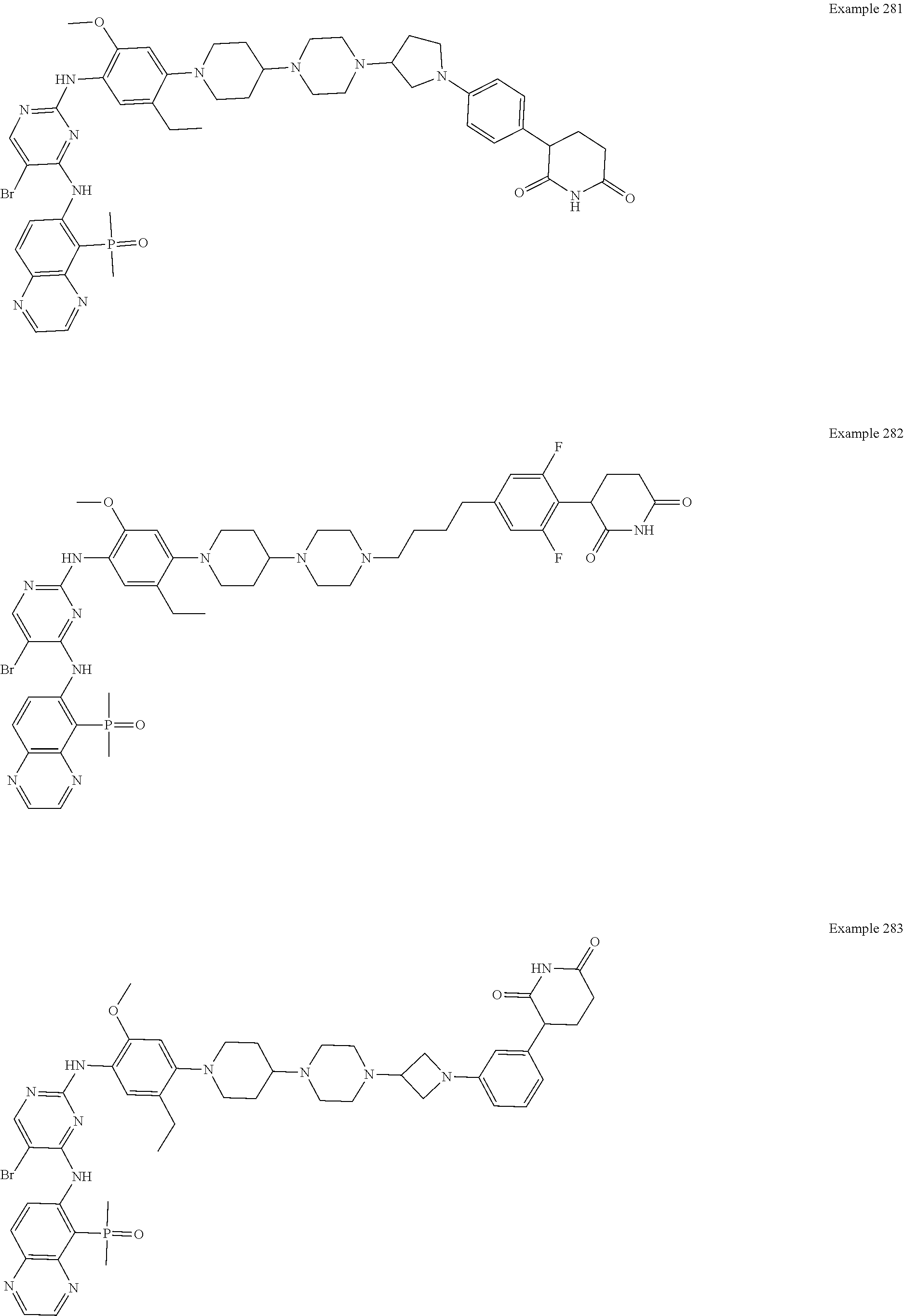 [SMILES: CCc1cc(Nc2ncc(Br)c(Nc3ccc4nccnc4c3P(C)(C)=O)n2)c(OC)cc1N1CCC(N2CCN(C3CCN(c4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(CCCCc3cc(F)c(C4CCC(=O)NC4=O)c(F)c3)CC2)CC1 ; CCc1cc(Nc2ncc(Br)c(Nc3ccc4nccnc4c3P(C)(C)=O)n2)c(OC)cc1N1CCC(N2CCN(C3CN(c4cccc(C5CCC(=O)NC5=O)c4)C3)CC2)CC1]

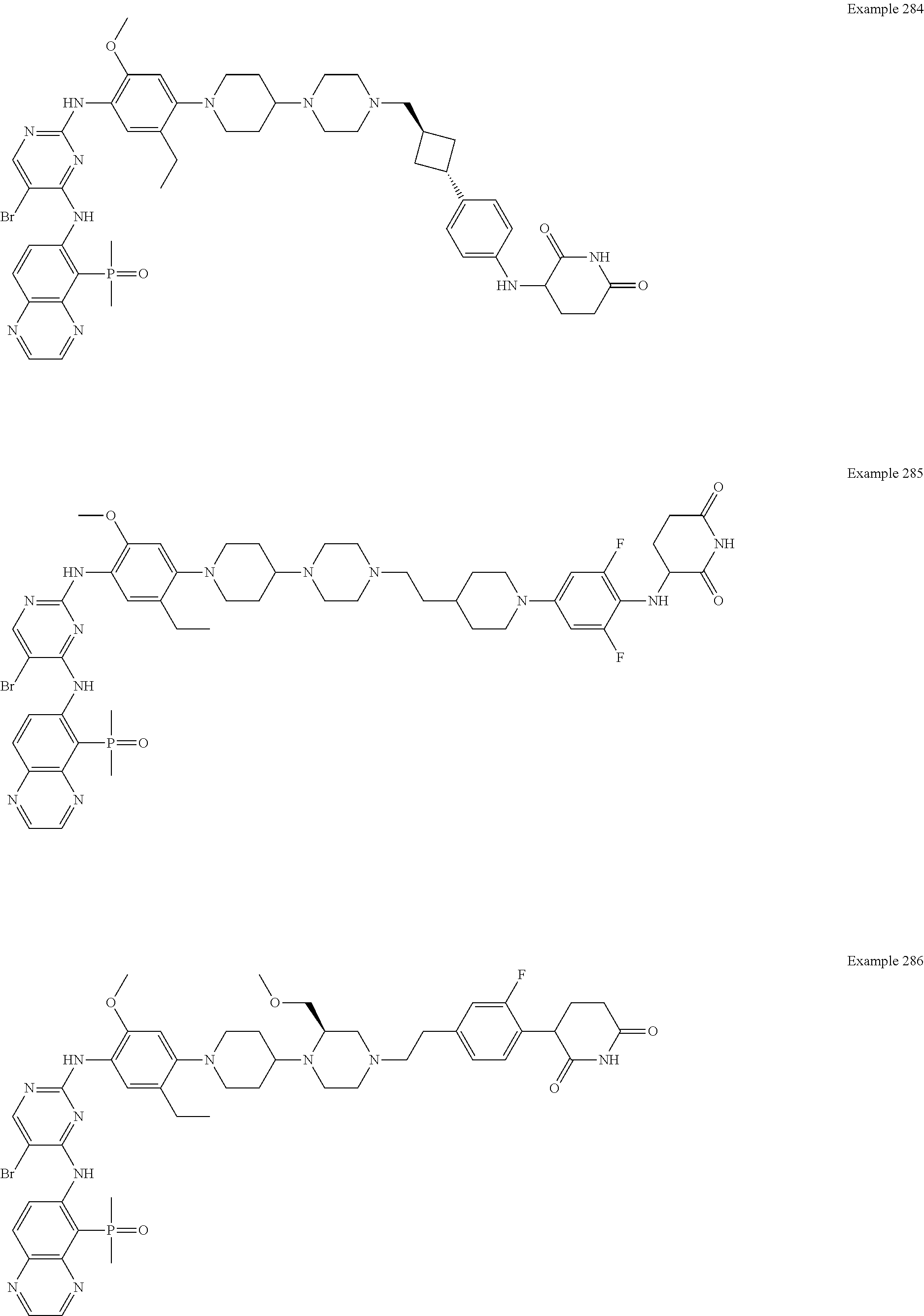 [SMILES: CCc1cc(Nc2ncc(Br)c(Nc3ccc4nccnc4c3P(C)(C)=O)n2)c(OC)cc1N1CCC(N2CCN(C[C@H]3C[C@H](c4ccc(NC5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4cc(F)c(NC5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(F)c3)C[C@@H]2COC)CC1]

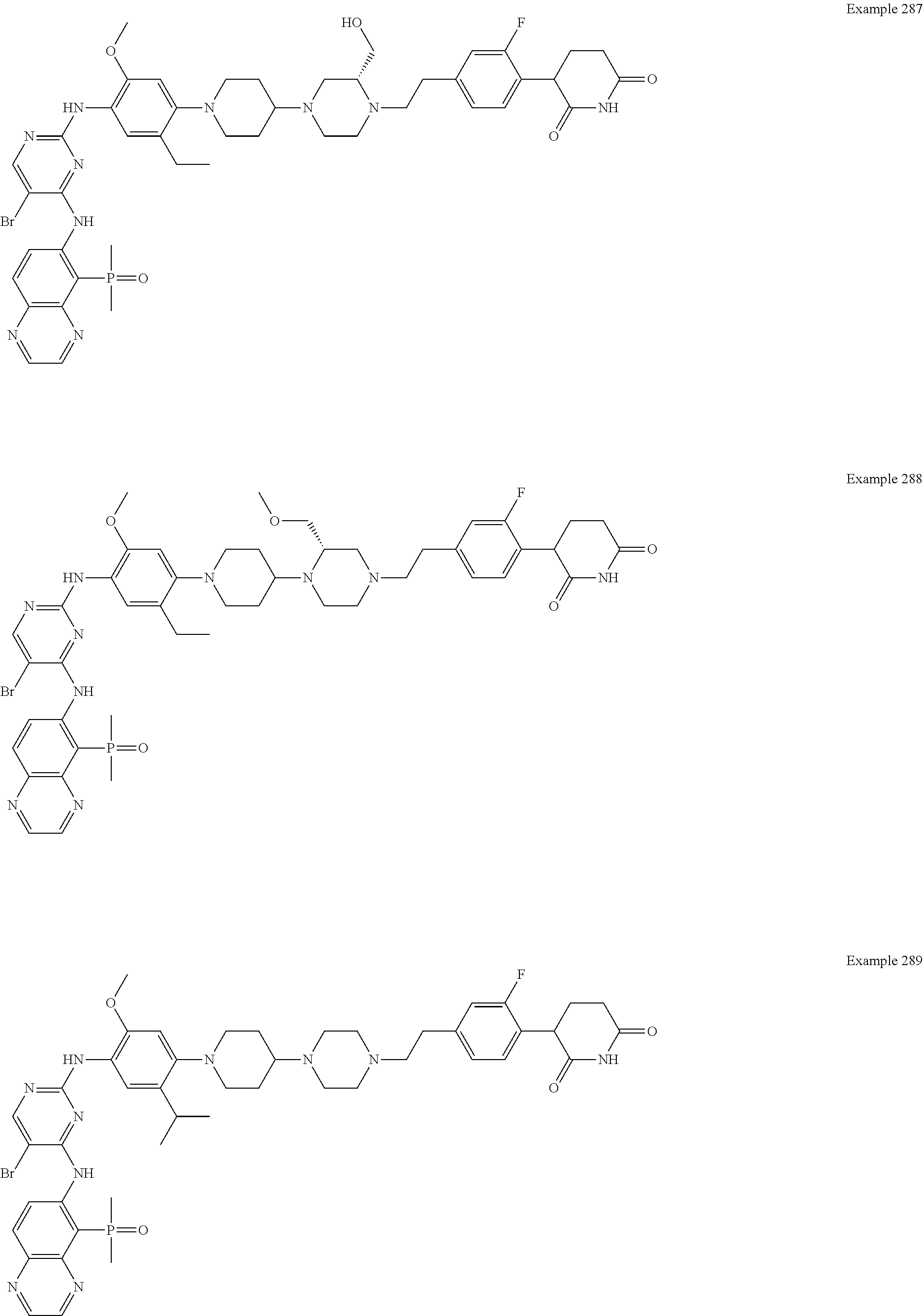 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(F)c3)[C@@H](CO)C2)CC1 ; CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(F)c3)C[C@H]2COC)CC1 ; COc1cc(N2CCC(N3CCN(CCc4ccc(C5CCC(=O)NC5=O)c(F)c4)CC3)CC2)c(C(C)C)cc1Nc1ncc(Br)c(Nc2ccc3nccnc3c2P(C)(C)=O)n1]

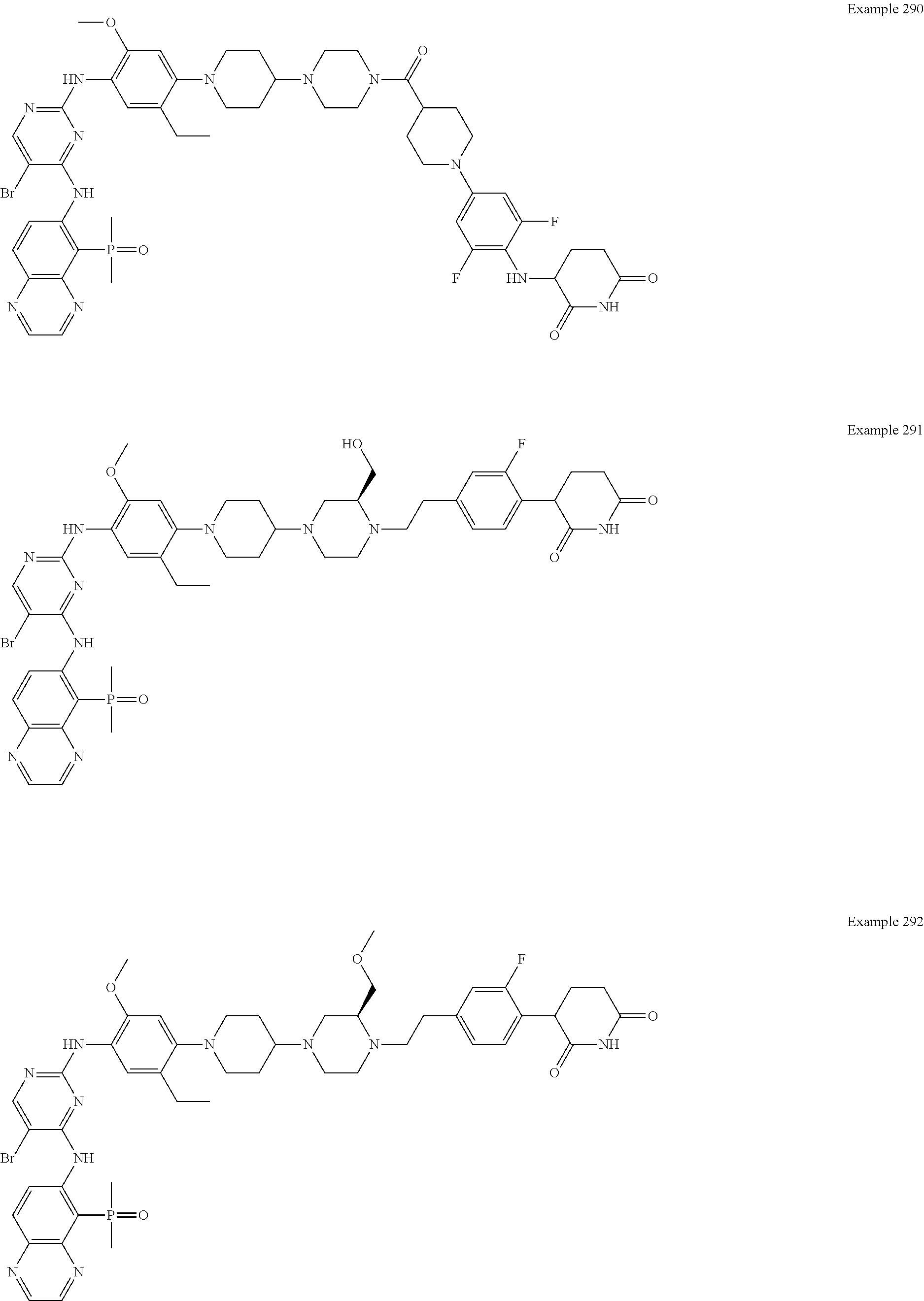 [SMILES: CCc1cc(Nc2ncc(Br)c(Nc3ccc4nccnc4c3P(C)(C)=O)n2)c(OC)cc1N1CCC(N2CCN(C(=O)C3CCN(c4cc(F)c(NC5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(F)c3)[C@H](CO)C2)CC1 ; CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(F)c3)[C@H](COC)C2)CC1]

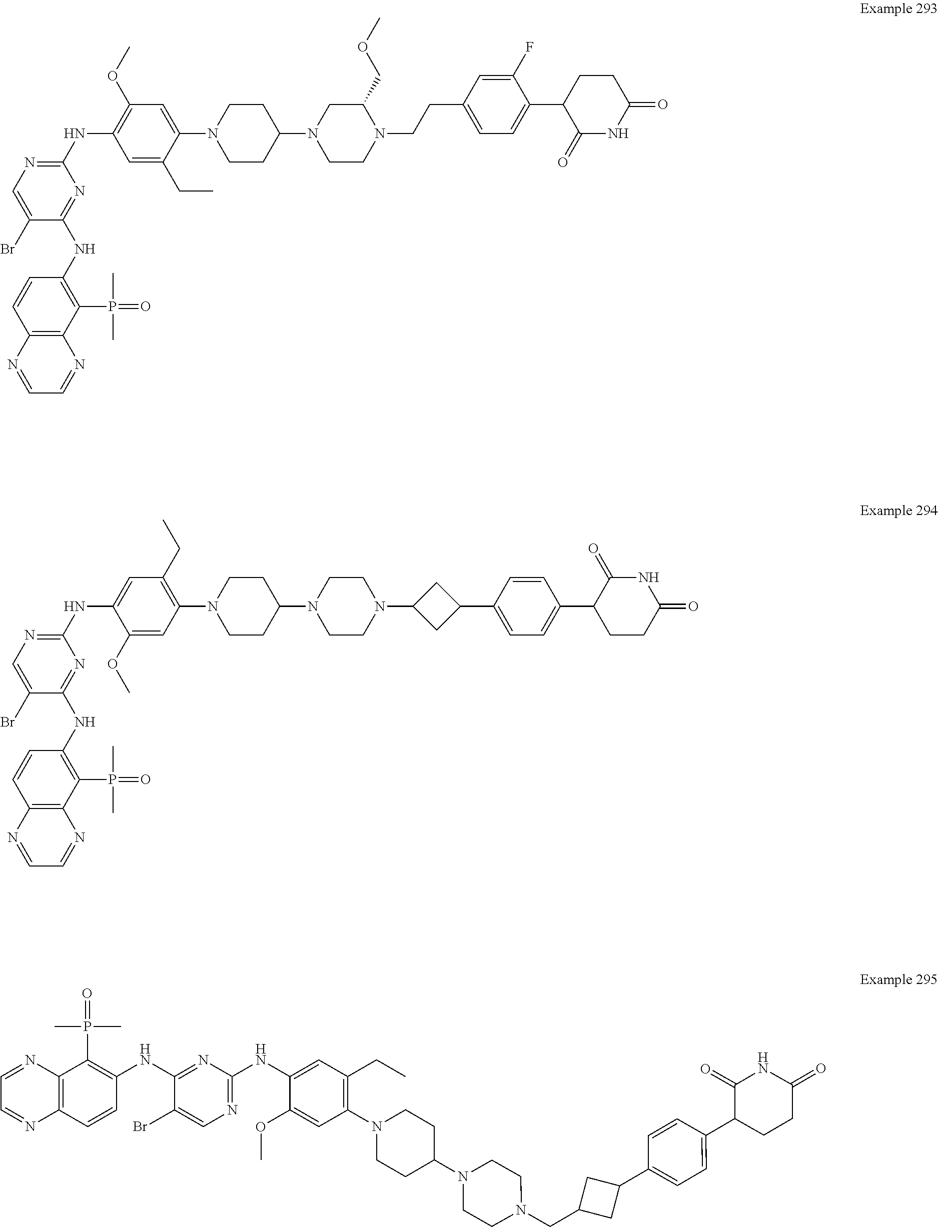 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(F)c3)[C@@H](COC)C2)CC1 ; CCc1cc(Nc2ncc(Br)c(Nc3ccc4nccnc4c3P(C)(C)=O)n2)c(OC)cc1N1CCC(N2CCN(C3CC(c4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(CC3CC(c4ccc(C5CCC(=O)NC5=O)cc4)C3)CC2)CC1]

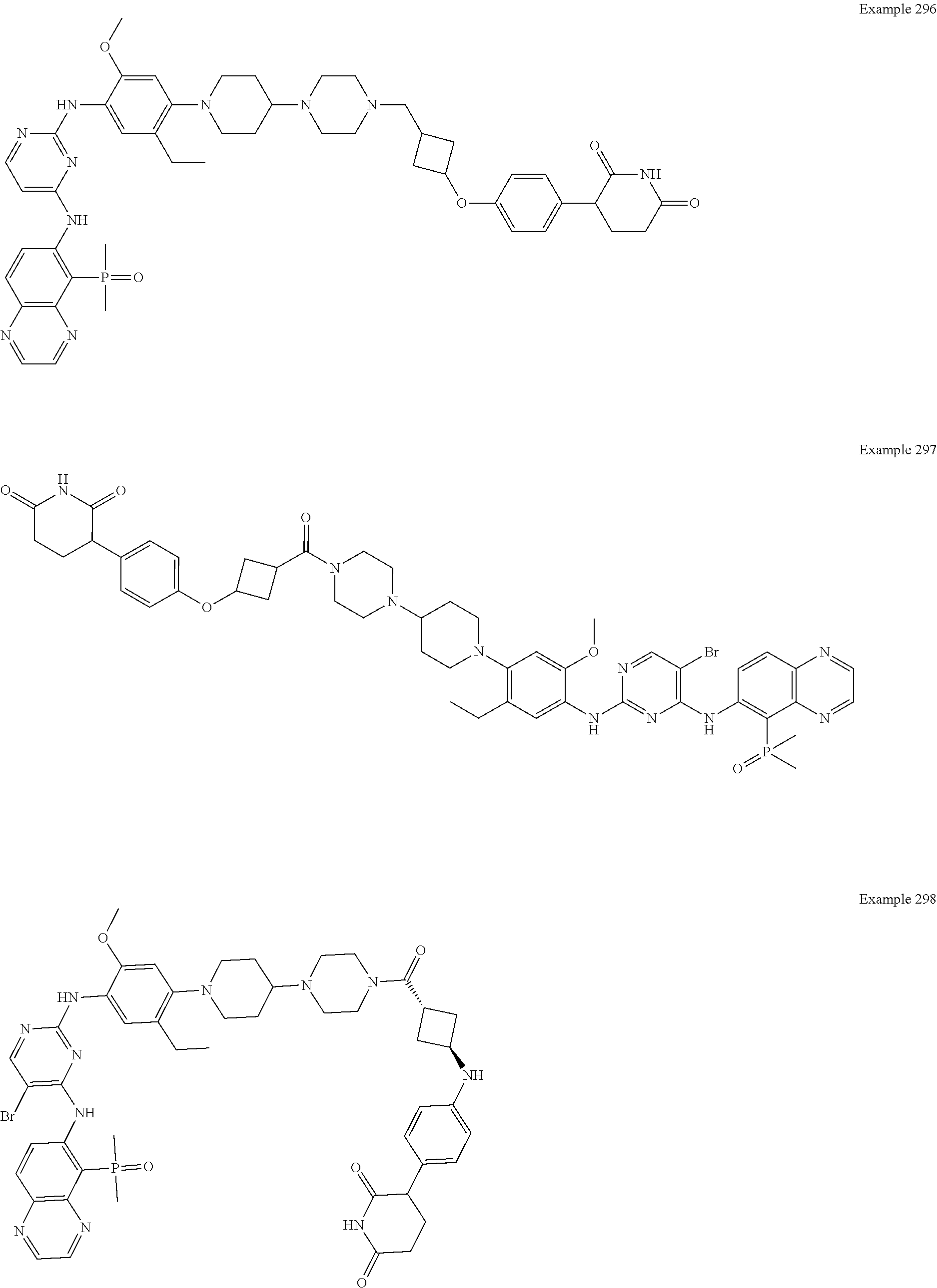 [SMILES: CCc1cc(Nc2nccc(Nc3ccc4nccnc4c3P(C)(C)=O)n2)c(OC)cc1N1CCC(N2CCN(CC3CC(Oc4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(C(=O)C3CC(Oc4ccc(C5CCC(=O)NC5=O)cc4)C3)CC2)CC1 ; CCc1cc(Nc2ncc(Br)c(Nc3ccc4nccnc4c3P(C)(C)=O)n2)c(OC)cc1N1CCC(N2CCN(C(=O)[C@H]3C[C@H](Nc4ccc(C5CCC(=O)NC5=O)cc4)C3)CC2)CC1]

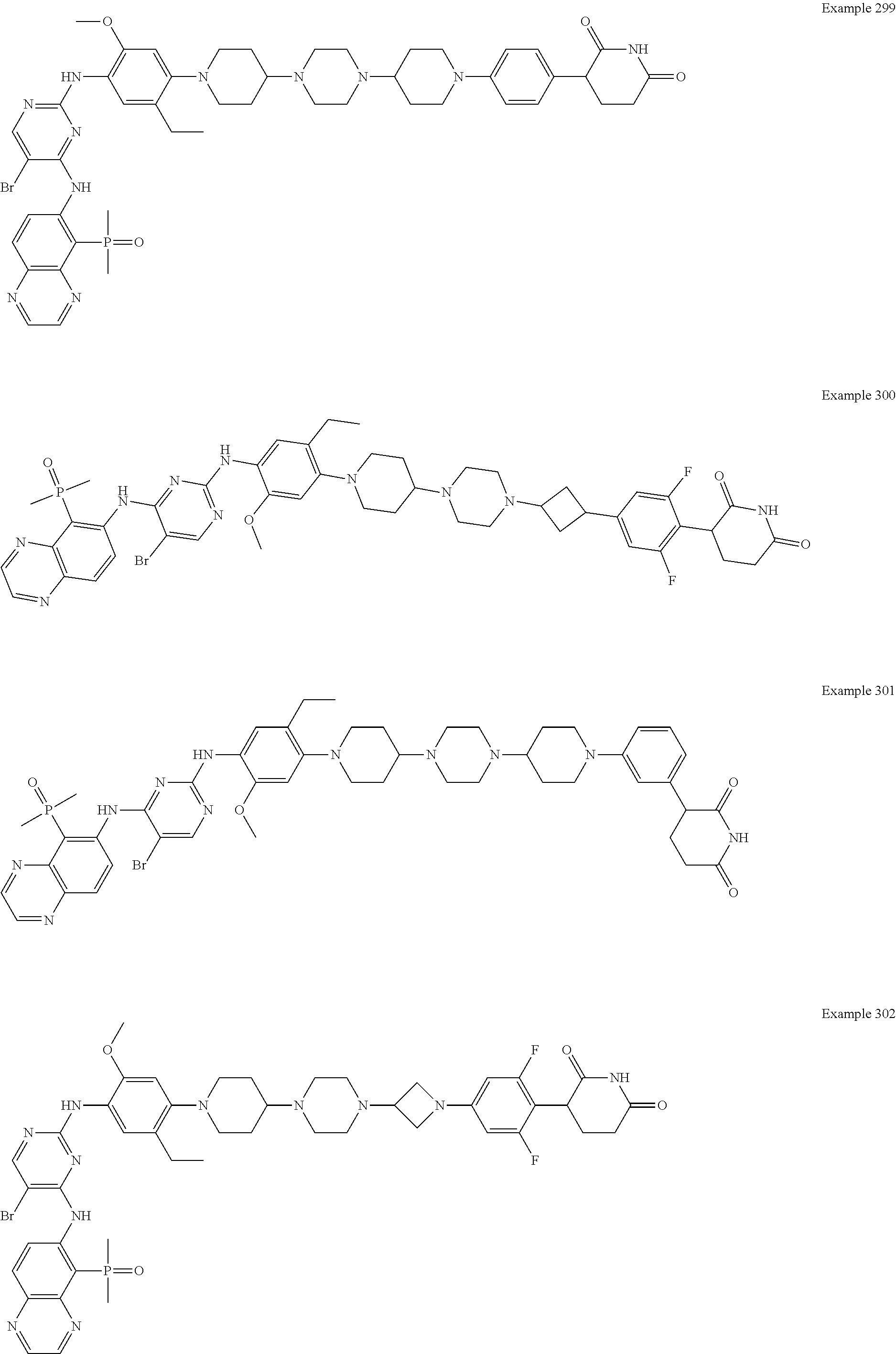 [SMILES: CCc1cc(Nc2ncc(Br)c(Nc3ccc4nccnc4c3P(C)(C)=O)n2)c(OC)cc1N1CCC(N2CCN(C3CCN(c4ccc(C5CCC(=O)NC5=O)cc4)CC3)CC2)CC1 ; CCc1cc(Nc2ncc(Br)c(Nc3ccc4nccnc4c3P(C)(C)=O)n2)c(OC)cc1N1CCC(N2CCN(C3CC(c4cc(F)c(C5CCC(=O)NC5=O)c(F)c4)C3)CC2)CC1 ; CCc1cc(Nc2ncc(Br)c(Nc3ccc4nccnc4c3P(C)(C)=O)n2)c(OC)cc1N1CCC(N2CCN(C3CCN(c4cccc(C5CCC(=O)NC5=O)c4)CC3)CC2)CC1 ; CCc1cc(Nc2ncc(Br)c(Nc3ccc4nccnc4c3P(C)(C)=O)n2)c(OC)cc1N1CCC(N2CCN(C3CN(c4cc(F)c(C5CCC(=O)NC5=O)c(F)c4)C3)CC2)CC1]

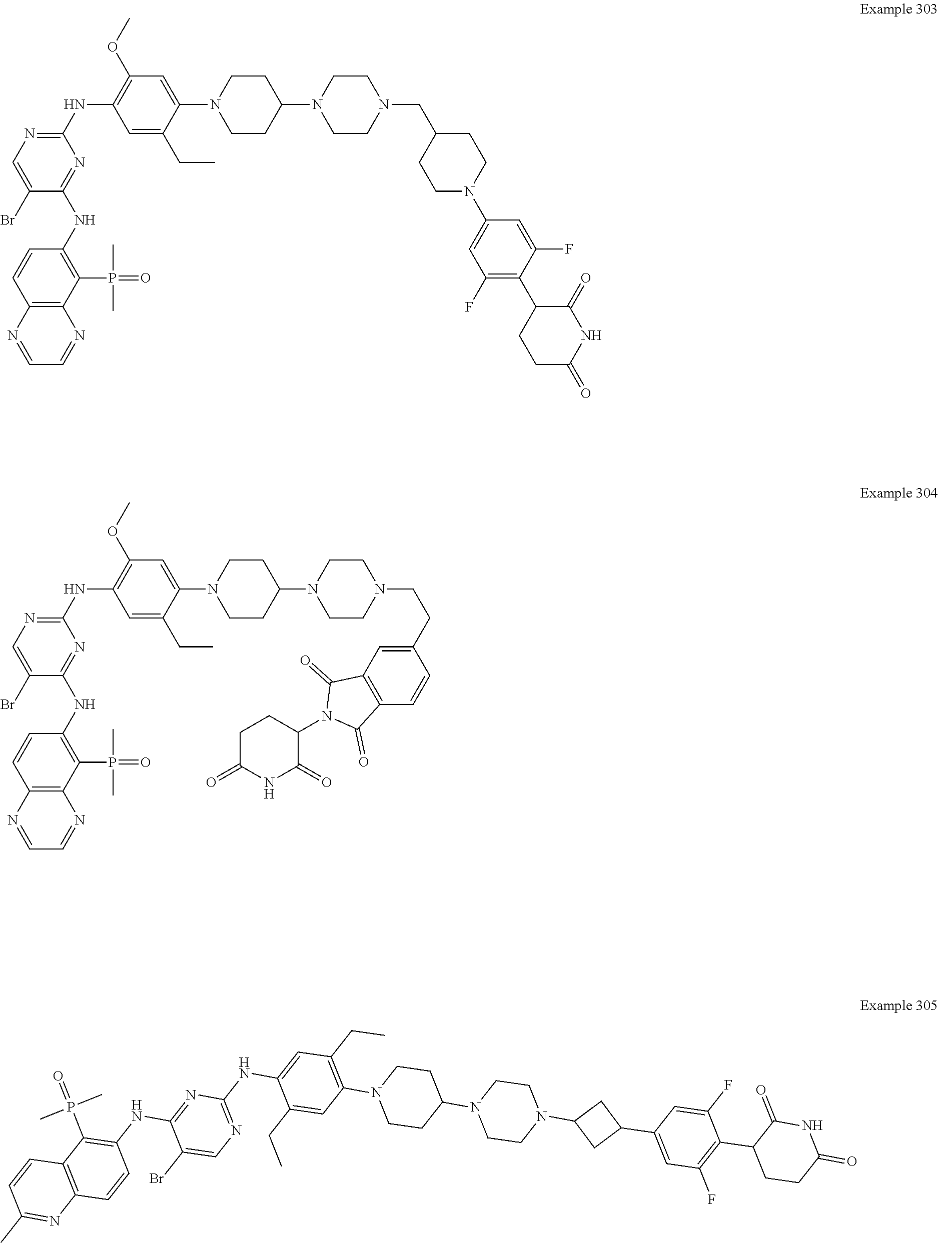 [SMILES: CCc1cc(Nc2ncc(Br)c(Nc3ccc4nccnc4c3P(C)(C)=O)n2)c(OC)cc1N1CCC(N2CCN(CC3CCN(c4cc(F)c(C5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(CCc3ccc4c(c3)C(=O)N(C3CCC(=O)NC3=O)C4=O)CC2)CC1 ; CCc1cc(N2CCC(N3CCN(C4CC(c5cc(F)c(C6CCC(=O)NC6=O)c(F)c5)C4)CC3)CC2)c(CC)cc1Nc1ncc(Br)c(Nc2ccc3nc(C)ccc3c2P(C)(C)=O)n1]

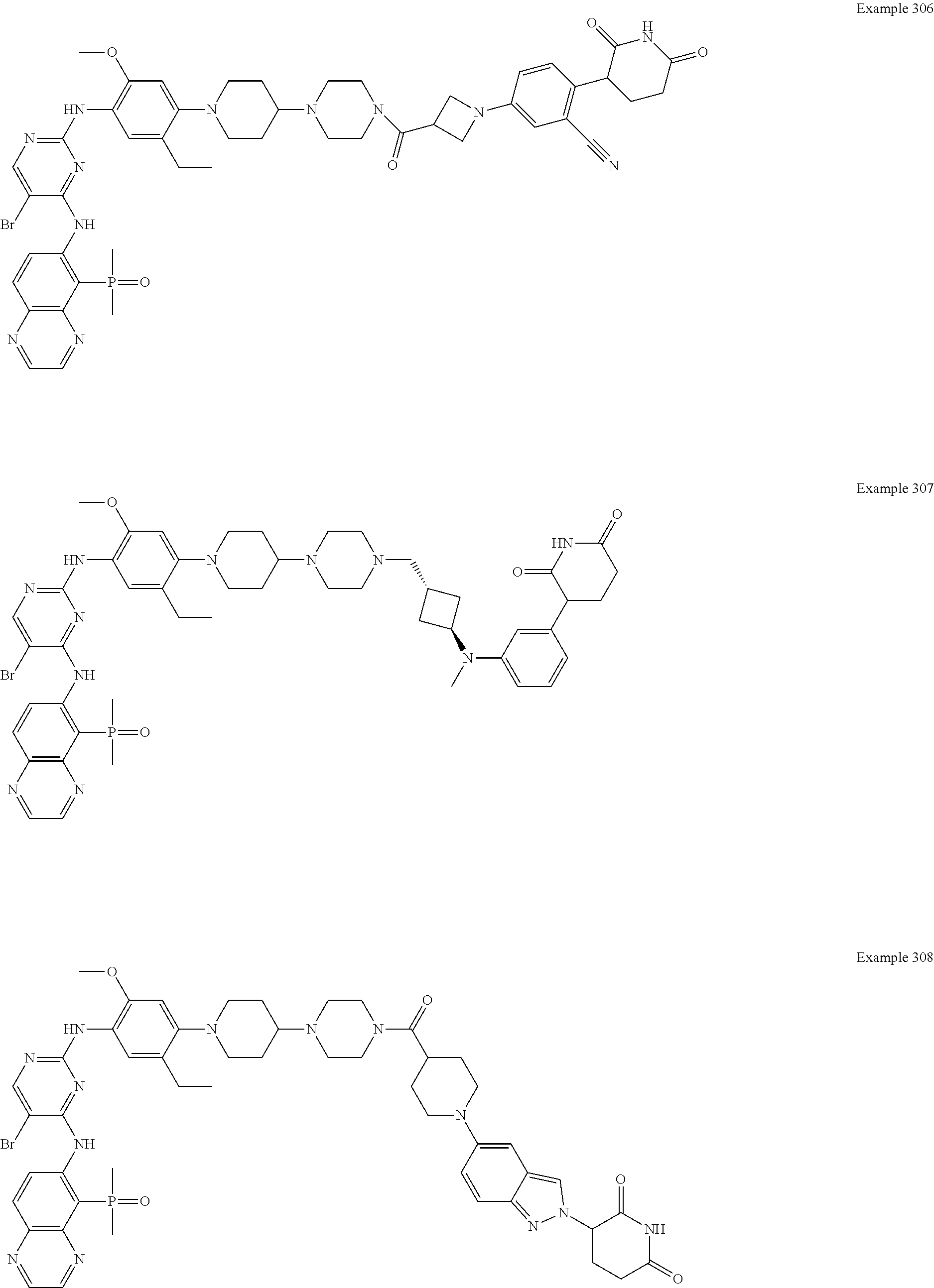 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc(C5CCC(=O)NC5=O)c(C#N)c4)C3)CC2)CC1 ; CCc1cc(Nc2ncc(Br)c(Nc3ccc4nccnc4c3P(C)(C)=O)n2)c(OC)cc1N1CCC(N2CCN(C[C@H]3C[C@H](N(C)c4cccc(C5CCC(=O)NC5=O)c4)C3)CC2)CC1 ; CCc1cc(Nc2ncc(Br)c(Nc3ccc4nccnc4c3P(C)(C)=O)n2)c(OC)cc1N1CCC(N2CCN(C(=O)C3CCN(c4ccc5nn(C6CCC(=O)NC6=O)cc5c4)CC3)CC2)CC1]

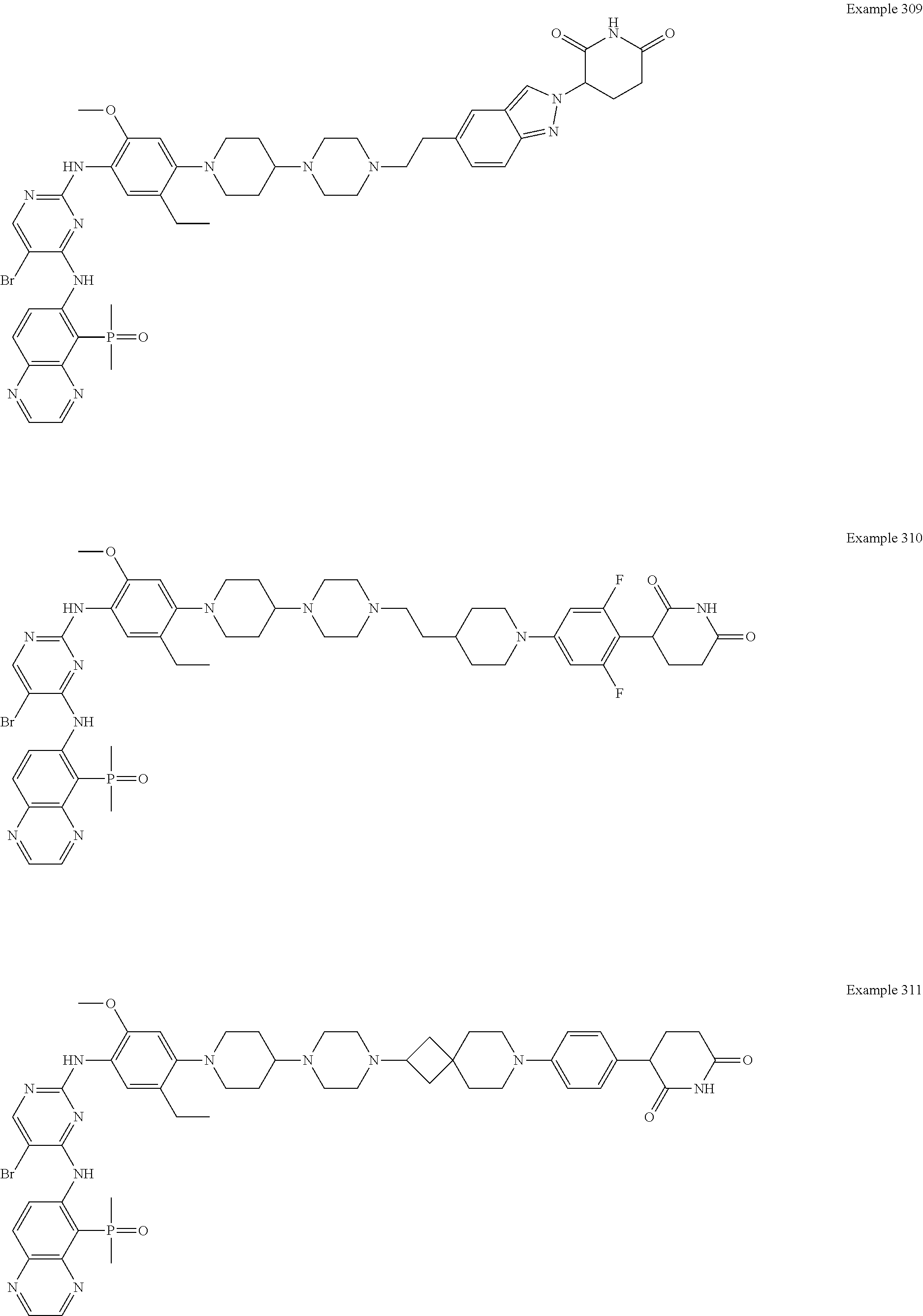 [SMILES: CCc1cc(Nc2ncc(Br)c(Nc3ccc4nccnc4c3P(C)(C)=O)n2)c(OC)cc1N1CCC(N2CCN(CCc3ccc4nn(C5CCC(=O)NC5=O)cc4c3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(C3CC4(CCN(c5ccc(C6CCC(=O)NC6=O)cc5)CC4)C3)CC2)CC1]

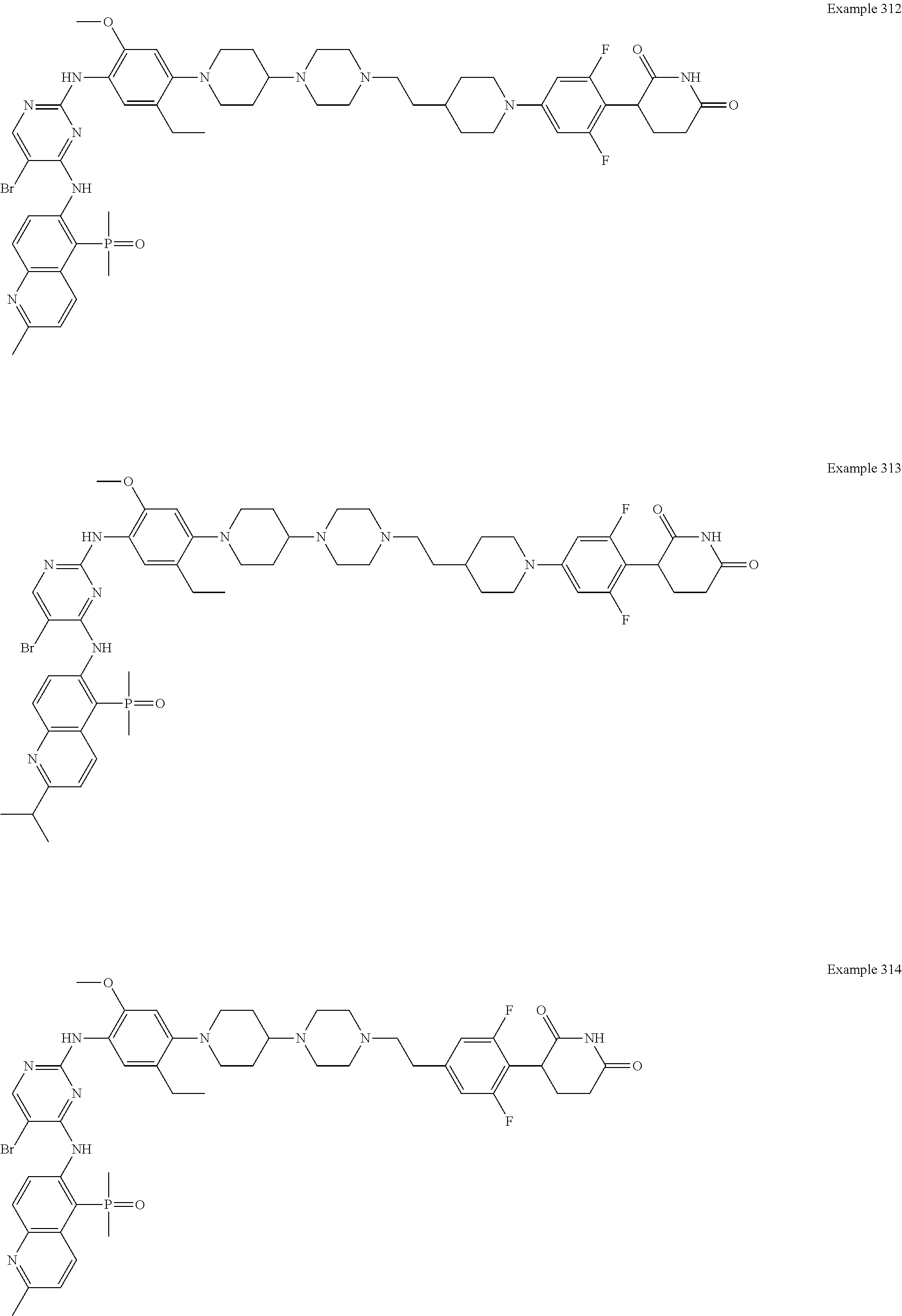 [SMILES: CCc1cc(Nc2ncc(Br)c(Nc3ccc4nc(C)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nc(C(C)C)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nc(C)ccc4c3P(C)(C)=O)n2)c(OC)cc1N1CCC(N2CCN(CCc3cc(F)c(C4CCC(=O)NC4=O)c(F)c3)CC2)CC1]

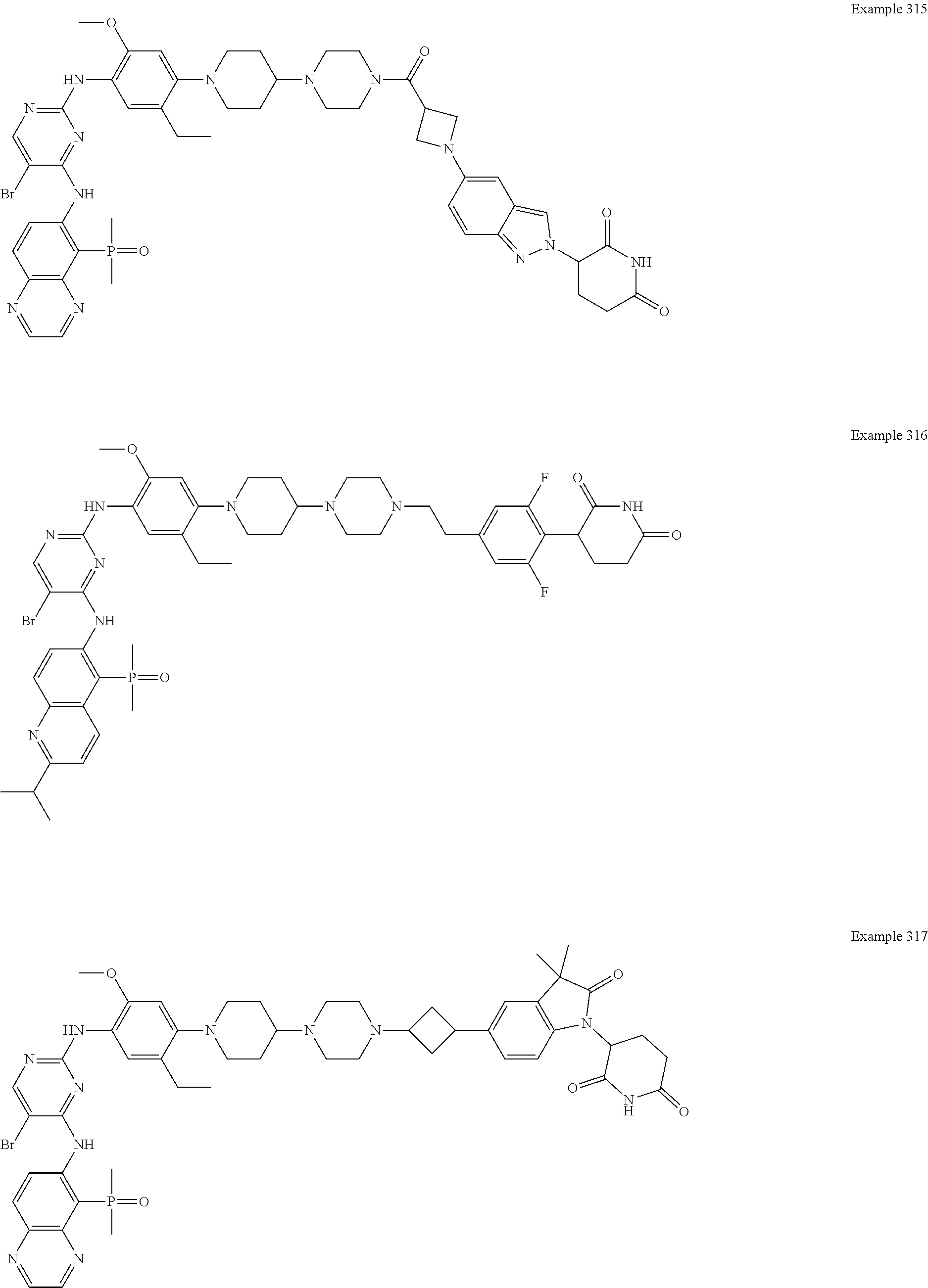 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc5nn(C6CCC(=O)NC6=O)cc5c4)C3)CC2)CC1 ; CCc1cc(Nc2ncc(Br)c(Nc3ccc4nc(C(C)C)ccc4c3P(C)(C)=O)n2)c(OC)cc1N1CCC(N2CCN(CCc3cc(F)c(C4CCC(=O)NC4=O)c(F)c3)CC2)CC1 ; CCc1cc(Nc2ncc(Br)c(Nc3ccc4nccnc4c3P(C)(C)=O)n2)c(OC)cc1N1CCC(N2CCN(C3CC(c4ccc5c(c4)C(C)(C)C(=O)N5C4CCC(=O)NC4=O)C3)CC2)CC1]

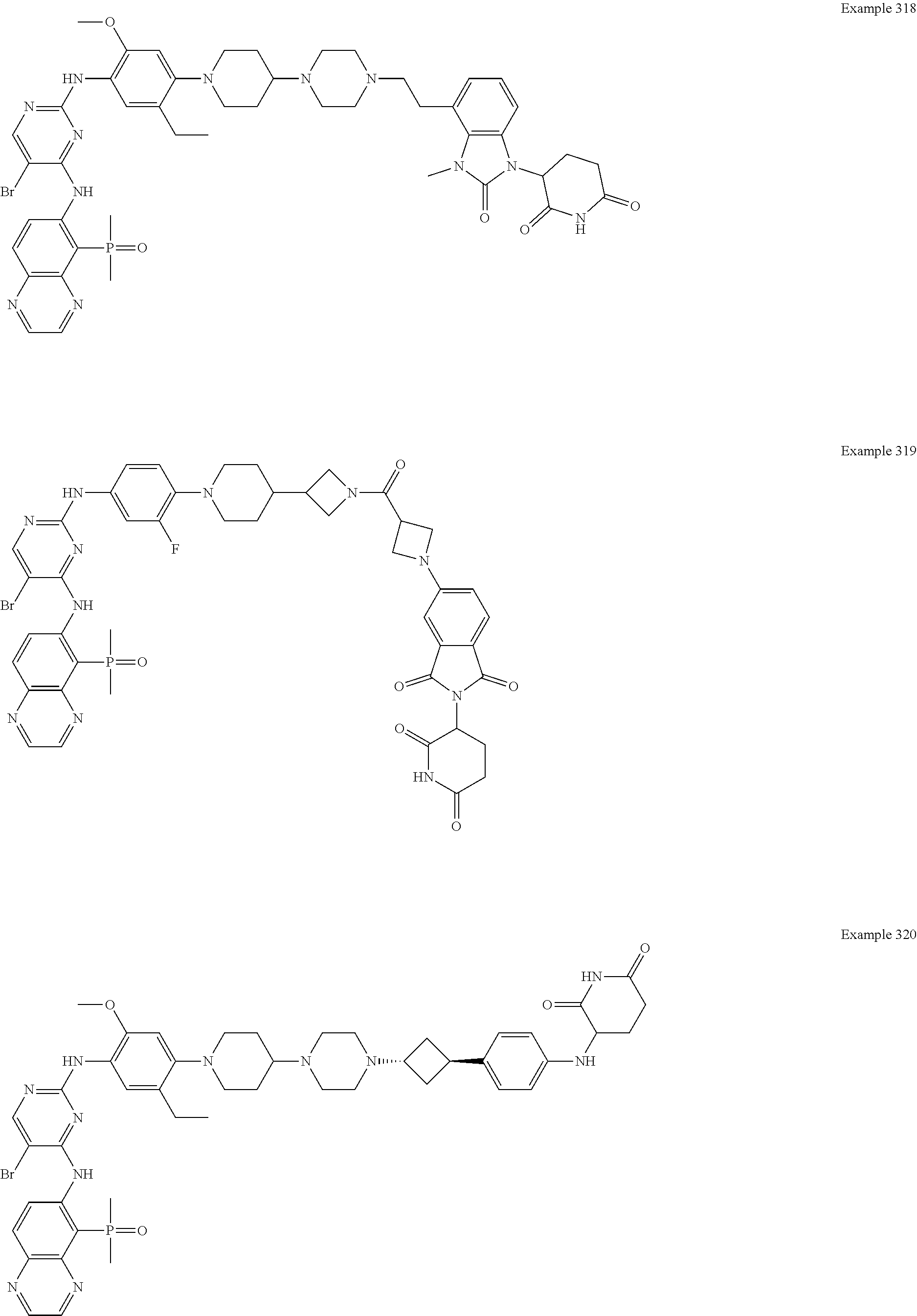 [SMILES: CCc1cc(Nc2ncc(Br)c(Nc3ccc4nccnc4c3P(C)(C)=O)n2)c(OC)cc1N1CCC(N2CCN(CCc3cccc4c3n(C)c(=O)n4C3CCC(=O)NC3=O)CC2)CC1 ; CP(C)(=O)c1c(Nc2nc(Nc3ccc(N4CCC(C5CN(C(=O)C6CN(c7ccc8c(c7)C(=O)N(C7CCC(=O)NC7=O)C8=O)C6)C5)CC4)c(F)c3)ncc2Br)ccc2nccnc12 ; CCc1cc(Nc2ncc(Br)c(Nc3ccc4nccnc4c3P(C)(C)=O)n2)c(OC)cc1N1CCC(N2CCN([C@H]3C[C@H](c4ccc(NC5CCC(=O)NC5=O)cc4)C3)CC2)CC1]

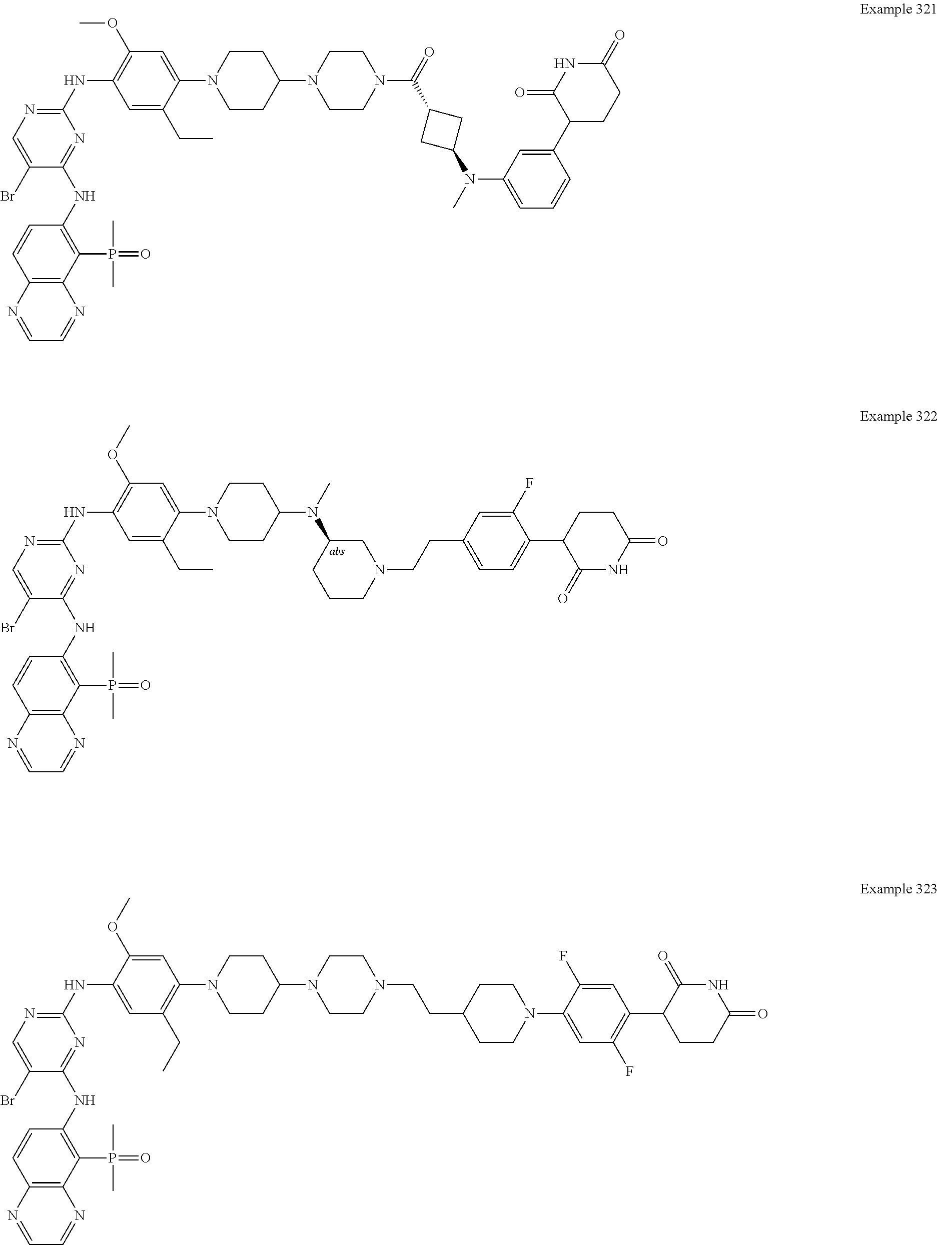 [SMILES: CCc1cc(Nc2ncc(Br)c(Nc3ccc4nccnc4c3P(C)(C)=O)n2)c(OC)cc1N1CCC(N2CCN(C(=O)[C@H]3C[C@H](N(C)c4cccc(C5CCC(=O)NC5=O)c4)C3)CC2)CC1 ; CCc1cc(Nc2ncc(Br)c(Nc3ccc4nccnc4c3P(C)(C)=O)n2)c(OC)cc1N1CCC(N(C)[C@@H]2CCCN(CCc3ccc(C4CCC(=O)NC4=O)c(F)c3)C2)CC1 ; CCc1cc(Nc2ncc(Br)c(Nc3ccc4nccnc4c3P(C)(C)=O)n2)c(OC)cc1N1CCC(N2CCN(CCC3CCN(c4cc(F)c(C5CCC(=O)NC5=O)cc4F)CC3)CC2)CC1]

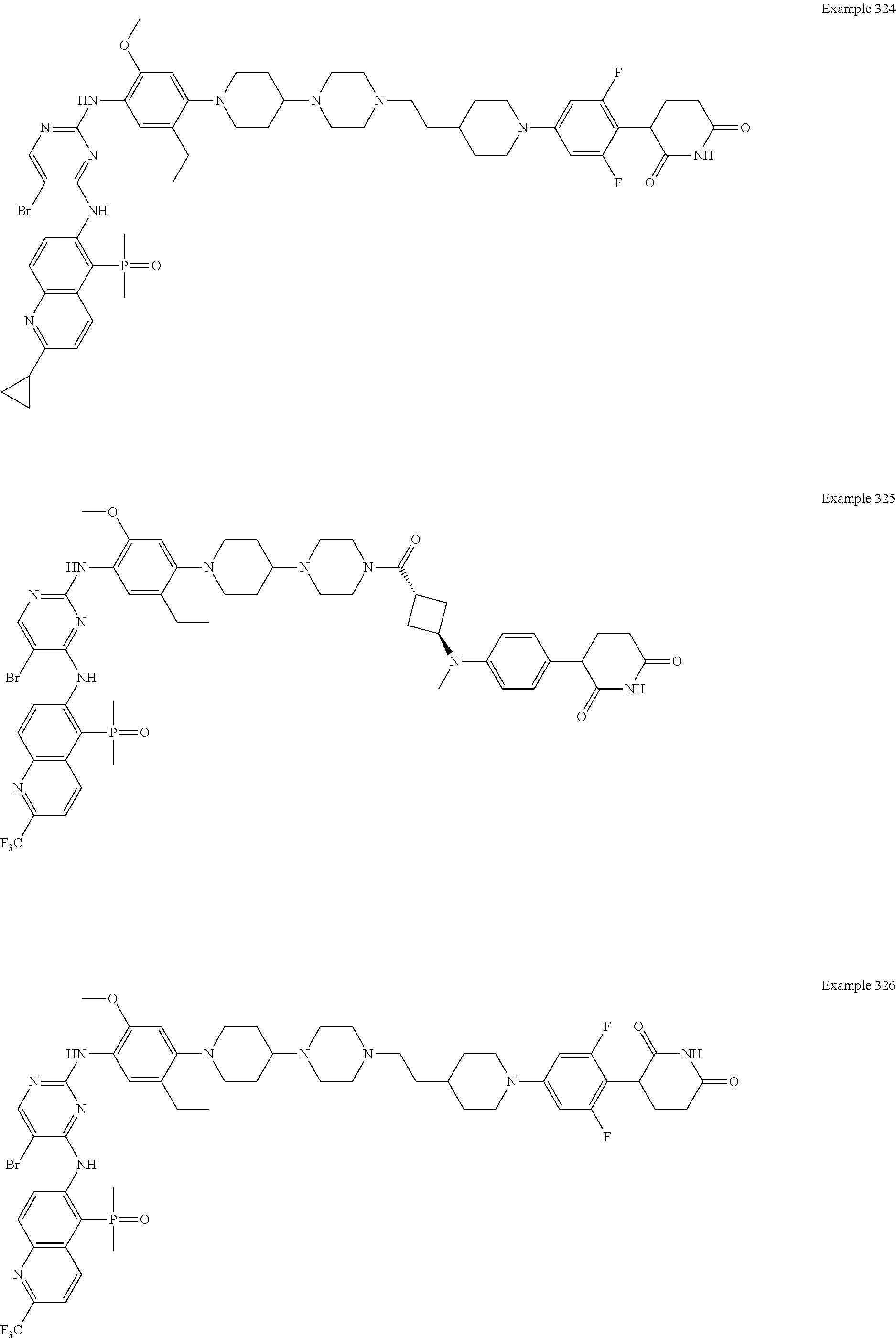 [SMILES: CCc1cc(Nc2ncc(Br)c(Nc3ccc4nc(C5CC5)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nc(C(F)(F)F)ccc4c3P(C)(C)=O)n2)c(OC)cc1N1CCC(N2CCN(C(=O)[C@H]3C[C@H](N(C)c4ccc(C5CCC(=O)NC5=O)cc4)C3)CC2)CC1 ; CCc1cc(Nc2ncc(Br)c(Nc3ccc4nc(C(F)(F)F)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1]

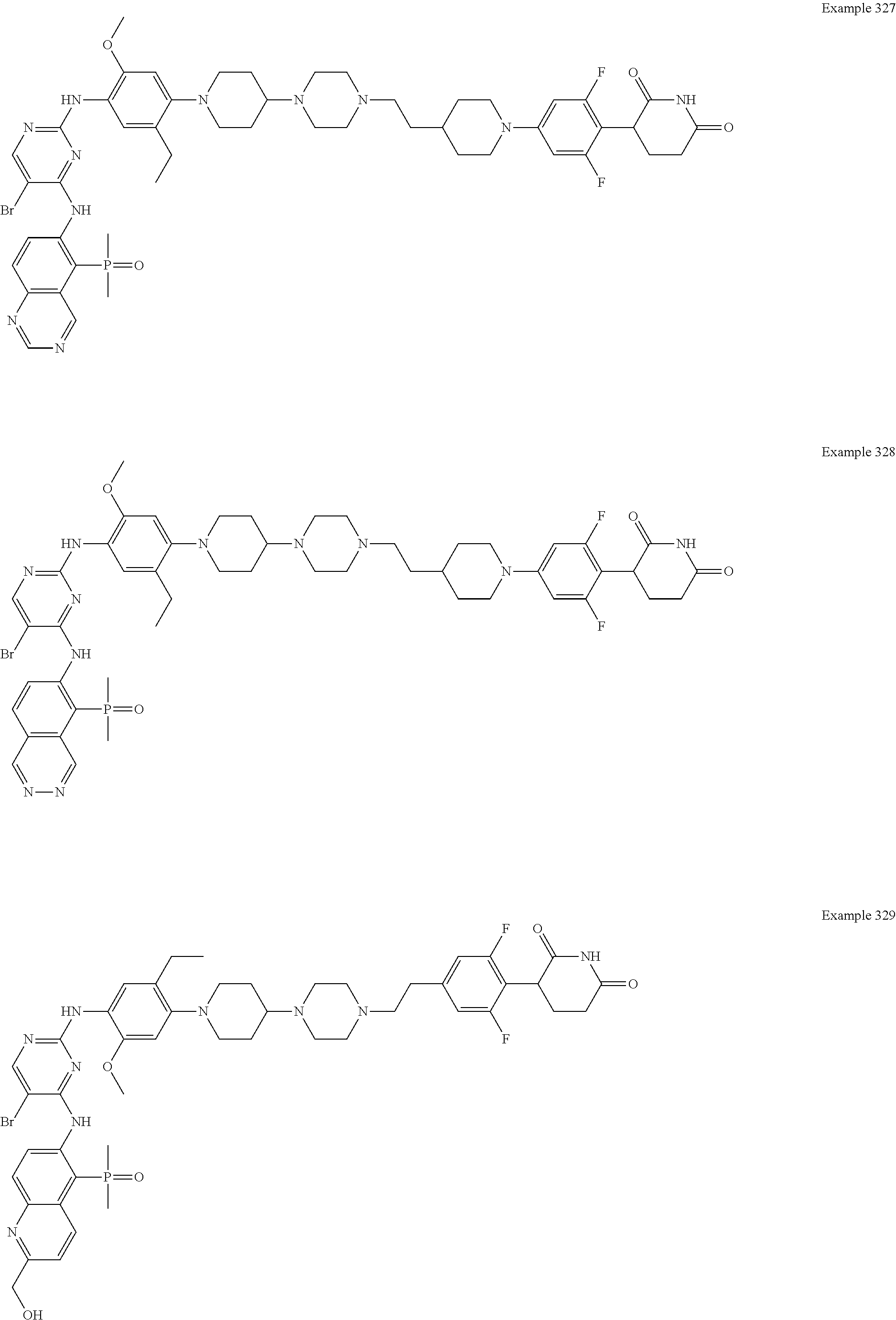 [SMILES: CCc1cc(Nc2ncc(Br)c(Nc3ccc4ncncc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4cnncc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nc(CO)ccc4c3P(C)(C)=O)n2)c(OC)cc1N1CCC(N2CCN(CCc3cc(F)c(C4CCC(=O)NC4=O)c(F)c3)CC2)CC1]

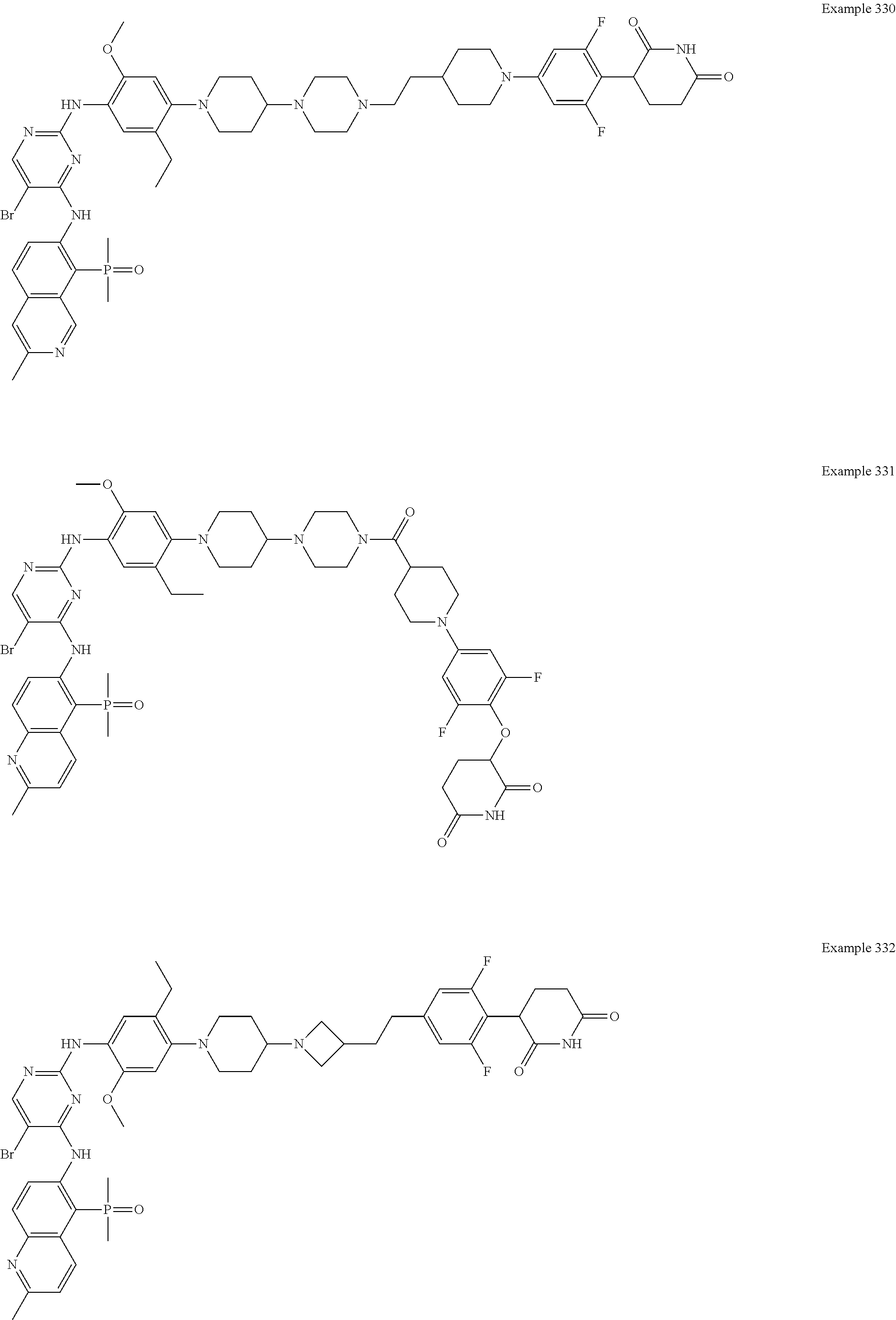 [SMILES: CCc1cc(Nc2ncc(Br)c(Nc3ccc4cc(C)ncc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CCN(c4cc(F)c(OC5CCC(=O)NC5=O)c(F)c4)CC3)CC2)CC1 ; CCc1cc(Nc2ncc(Br)c(Nc3ccc4nc(C)ccc4c3P(C)(C)=O)n2)c(OC)cc1N1CCC(N2CC(CCc3cc(F)c(C4CCC(=O)NC4=O)c(F)c3)C2)CC1]

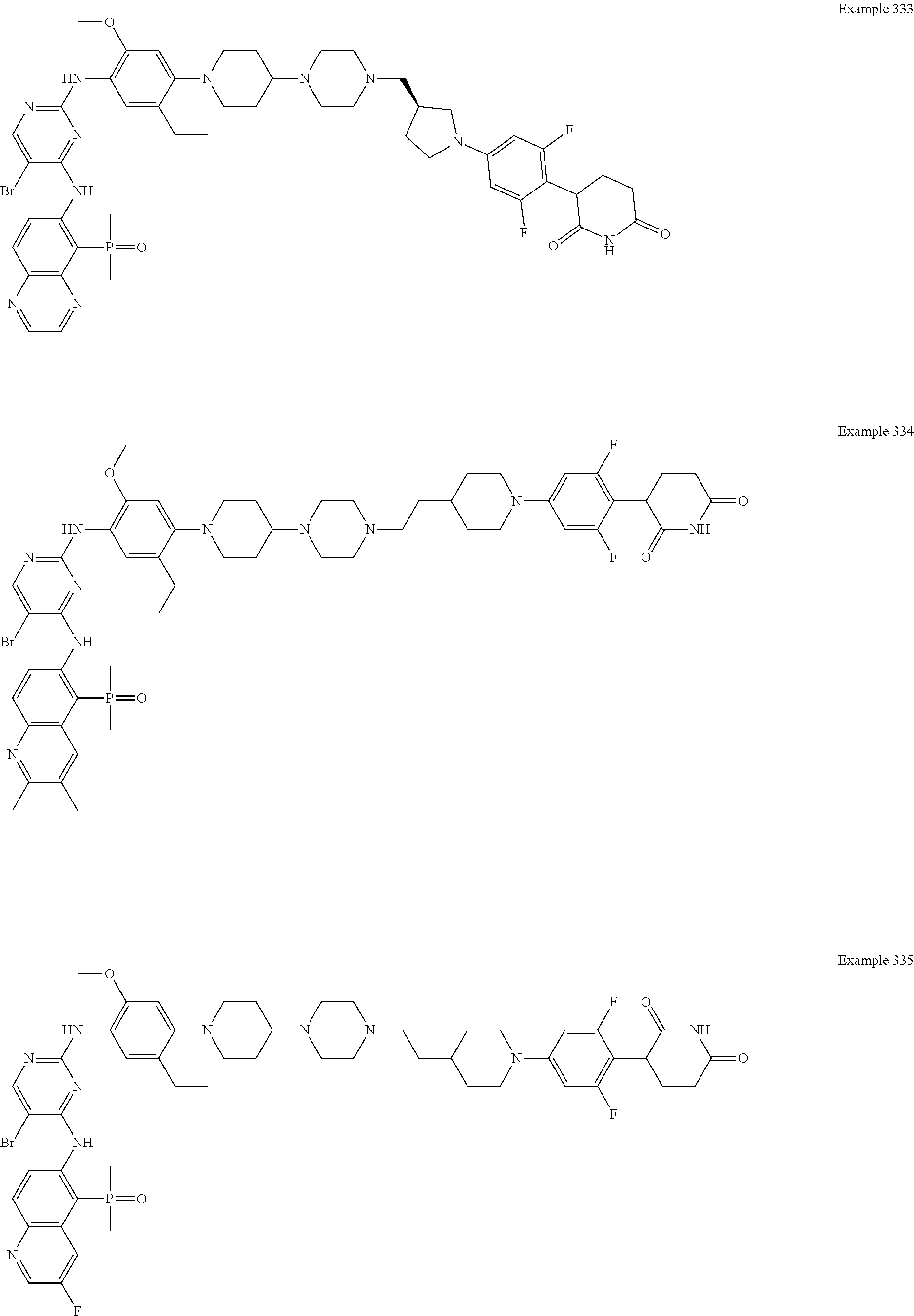 [SMILES: CCc1cc(Nc2ncc(Br)c(Nc3ccc4nccnc4c3P(C)(C)=O)n2)c(OC)cc1N1CCC(N2CCN(C[C@@H]3CCN(c4cc(F)c(C5CCC(=O)NC5=O)c(F)c4)C3)CC2)CC1 ; CCc1cc(Nc2ncc(Br)c(Nc3ccc4nc(C)c(C)cc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4ncc(F)cc4c3P(C)(C)=O)n2)c(OC)cc1N1CCC(N2CCN(CCC3CCN(c4cc(F)c(C5CCC(=O)NC5=O)c(F)c4)CC3)CC2)CC1]

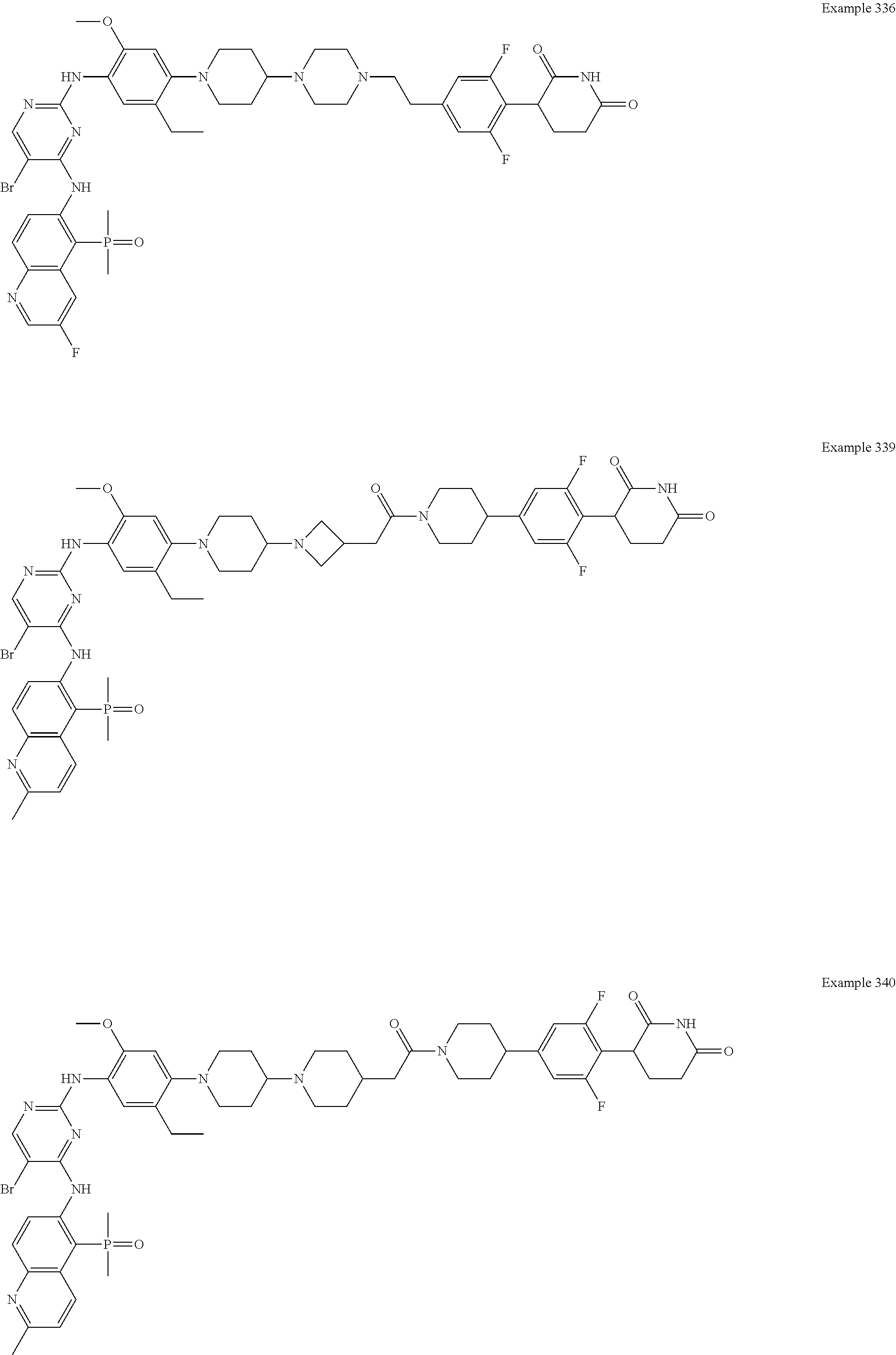 [SMILES: CCc1cc(Nc2ncc(Br)c(Nc3ccc4ncc(F)cc4c3P(C)(C)=O)n2)c(OC)cc1N1CCC(N2CCN(CCc3cc(F)c(C4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CC(CC(=O)N3CCC(c4cc(F)c(C5CCC(=O)NC5=O)c(F)c4)CC3)C2)CC1 ; CCc1cc(Nc2ncc(Br)c(Nc3ccc4nc(C)ccc4c3P(C)(C)=O)n2)c(OC)cc1N1CCC(N2CCC(CC(=O)N3CCC(c4cc(F)c(C5CCC(=O)NC5=O)c(F)c4)CC3)CC2)CC1]

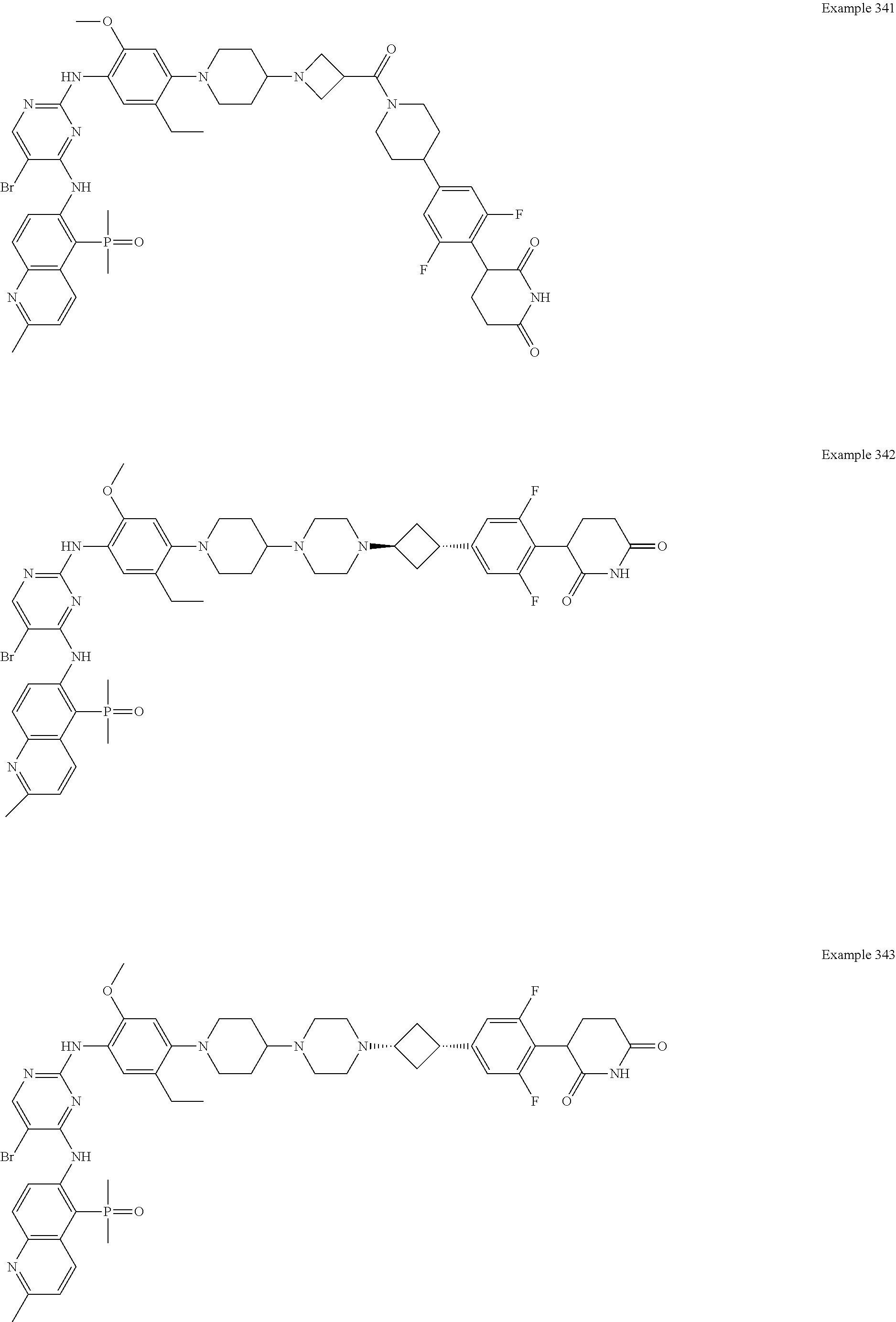 [SMILES: CCc1cc(Nc2ncc(Br)c(Nc3ccc4nc(C)ccc4c3P(C)(C)=O)n2)c(OC)cc1N1CCC(N2CC(C(=O)N3CCC(c4cc(F)c(C5CCC(=O)NC5=O)c(F)c4)CC3)C2)CC1 ; CCc1cc(Nc2ncc(Br)c(Nc3ccc4nc(C)ccc4c3P(C)(C)=O)n2)c(OC)cc1N1CCC(N2CCN([C@H]3C[C@H](c4cc(F)c(C5CCC(=O)NC5=O)c(F)c4)C3)CC2)CC1 ; CCc1cc(Nc2ncc(Br)c(Nc3ccc4nc(C)ccc4c3P(C)(C)=O)n2)c(OC)cc1N1CCC(N2CCN([C@H]3C[C@@H](c4cc(F)c(C5CCC(=O)NC5=O)c(F)c4)C3)CC2)CC1]

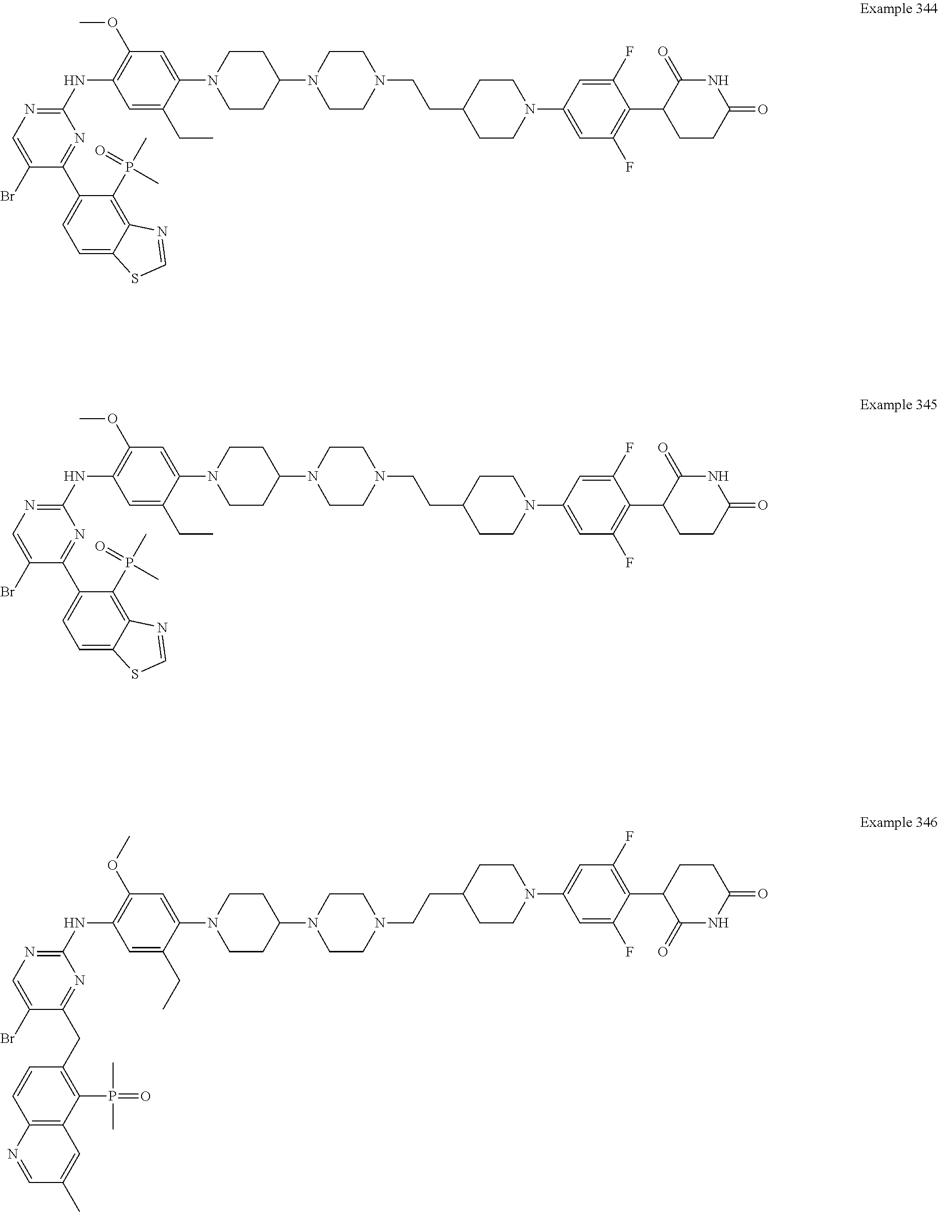 [SMILES: CCc1cc(Nc2ncc(Br)c(-c3ccc4scnc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(-c3ccc4scnc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Cc3ccc4ncc(C)cc4c3P(C)(C)=O)n2)c(OC)cc1N1CCC(N2CCN(CCC3CCN(c4cc(F)c(C5CCC(=O)NC5=O)c(F)c4)CC3)CC2)CC1]

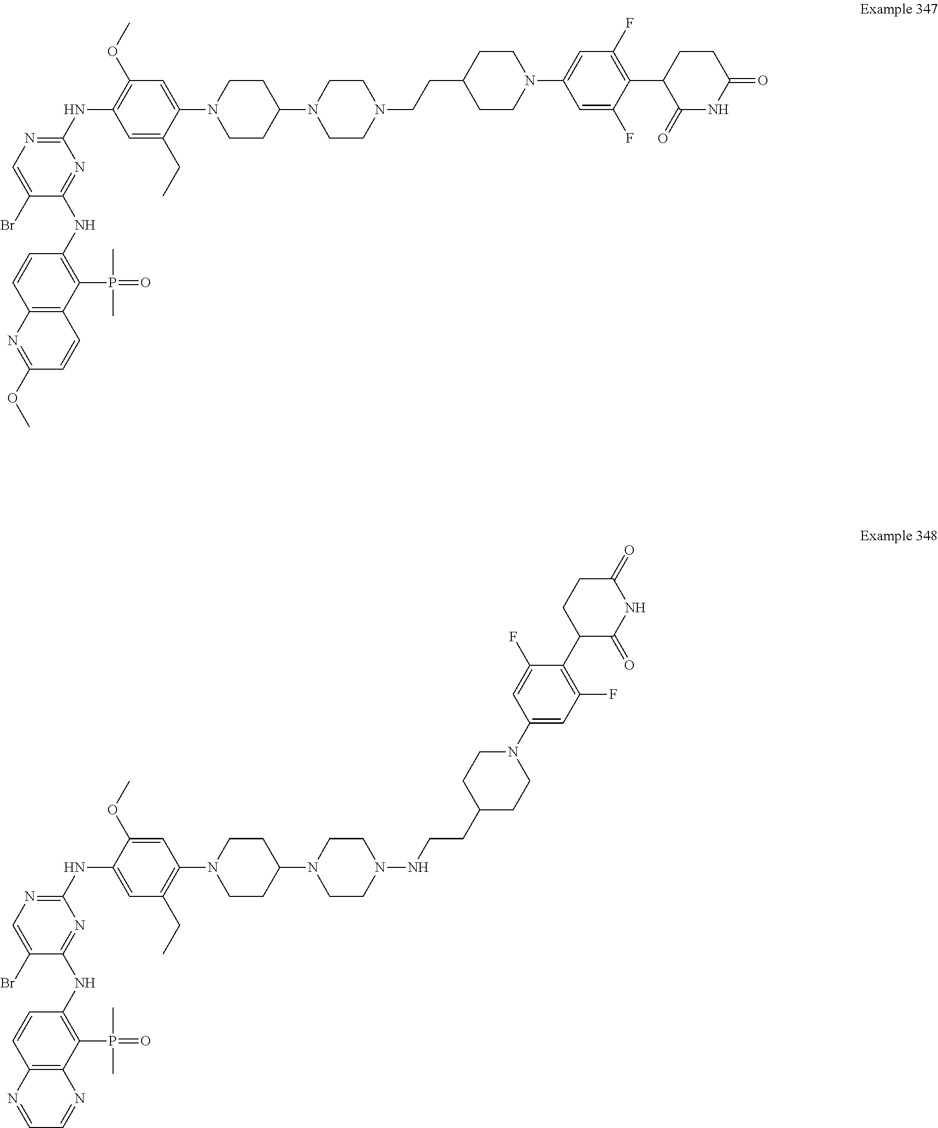 [SMILES: CCc1cc(Nc2ncc(Br)c(Nc3ccc4nc(OC)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nccnc4c3P(C)(C)=O)n2)c(OC)cc1N1CCC(N2CCN(NCCC3CCN(c4cc(F)c(C5CCC(=O)NC5=O)c(F)c4)CC3)CC2)CC1]

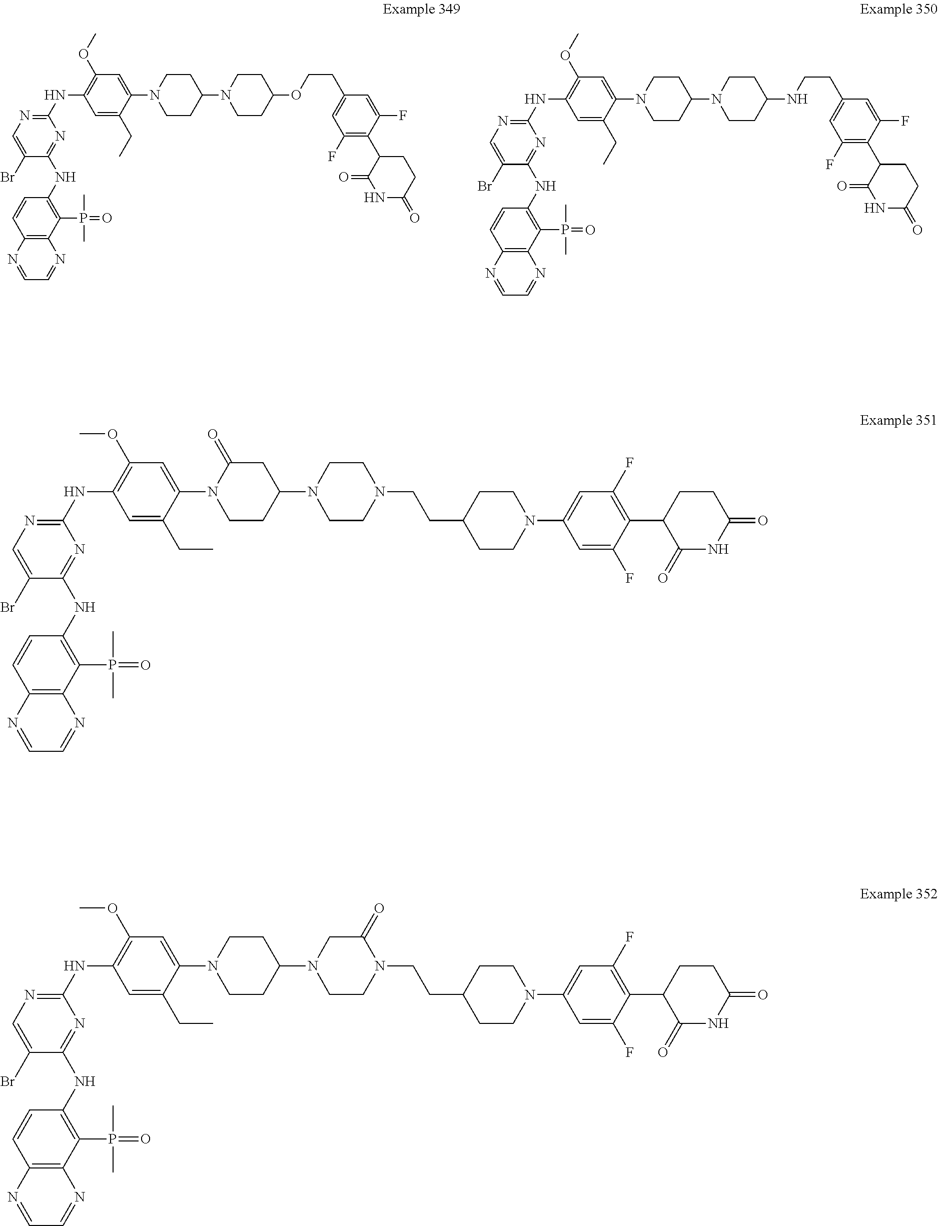 [SMILES: CCc1cc(Nc2ncc(Br)c(Nc3ccc4nccnc4c3P(C)(C)=O)n2)c(OC)cc1N1CCC(N2CCC(OCCc3cc(F)c(C4CCC(=O)NC4=O)c(F)c3)CC2)CC1 ; CCc1cc(Nc2ncc(Br)c(Nc3ccc4nccnc4c3P(C)(C)=O)n2)c(OC)cc1N1CCC(N2CCC(NCCc3cc(F)c(C4CCC(=O)NC4=O)c(F)c3)CC2)CC1 ; CCc1cc(Nc2ncc(Br)c(Nc3ccc4nccnc4c3P(C)(C)=O)n2)c(OC)cc1N1CCC(N2CCN(CCC3CCN(c4cc(F)c(C5CCC(=O)NC5=O)c(F)c4)CC3)CC2)CC1=O ; CCc1cc(Nc2ncc(Br)c(Nc3ccc4nccnc4c3P(C)(C)=O)n2)c(OC)cc1N1CCC(N2CCN(CCC3CCN(c4cc(F)c(C5CCC(=O)NC5=O)c(F)c4)CC3)C(=O)C2)CC1]

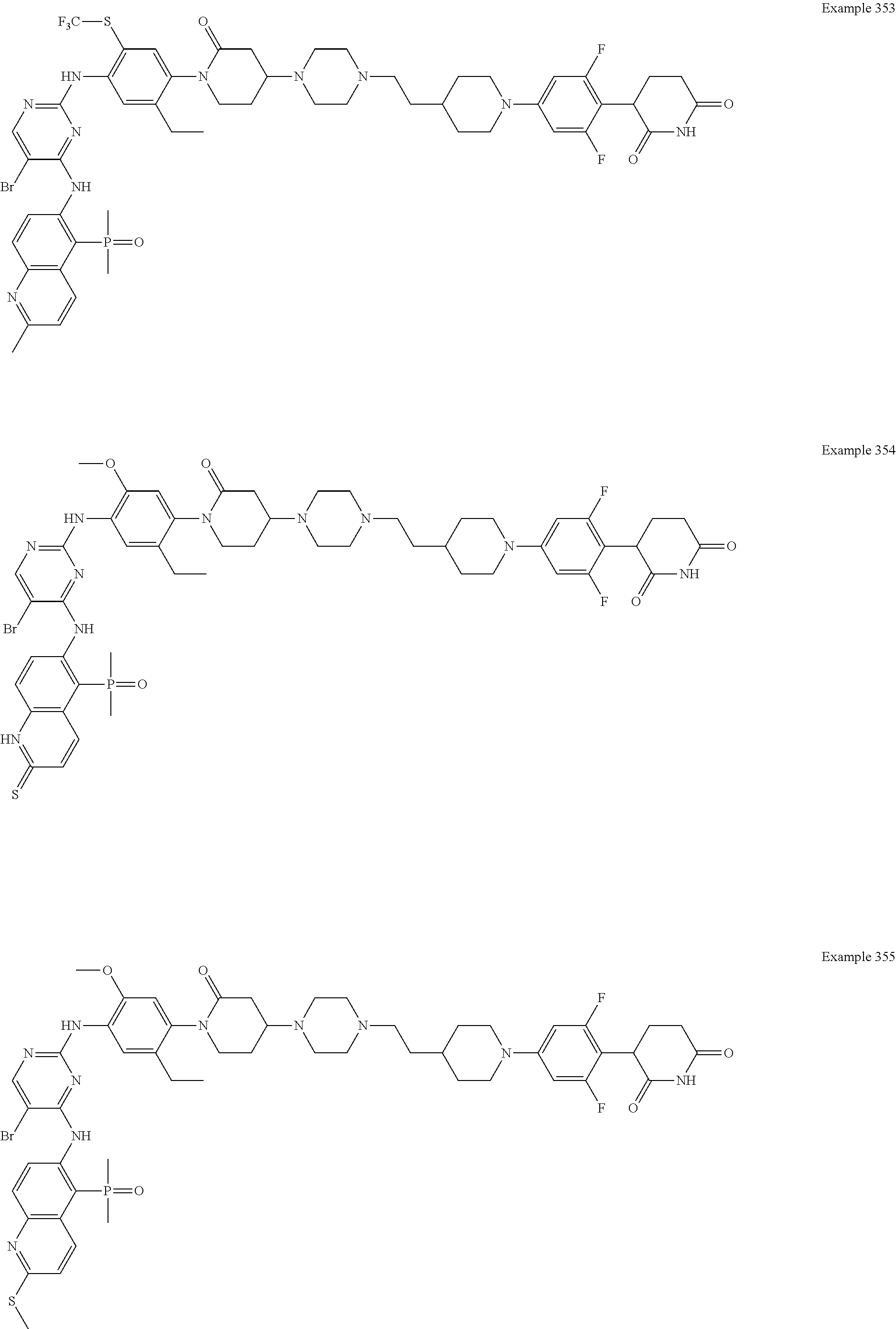

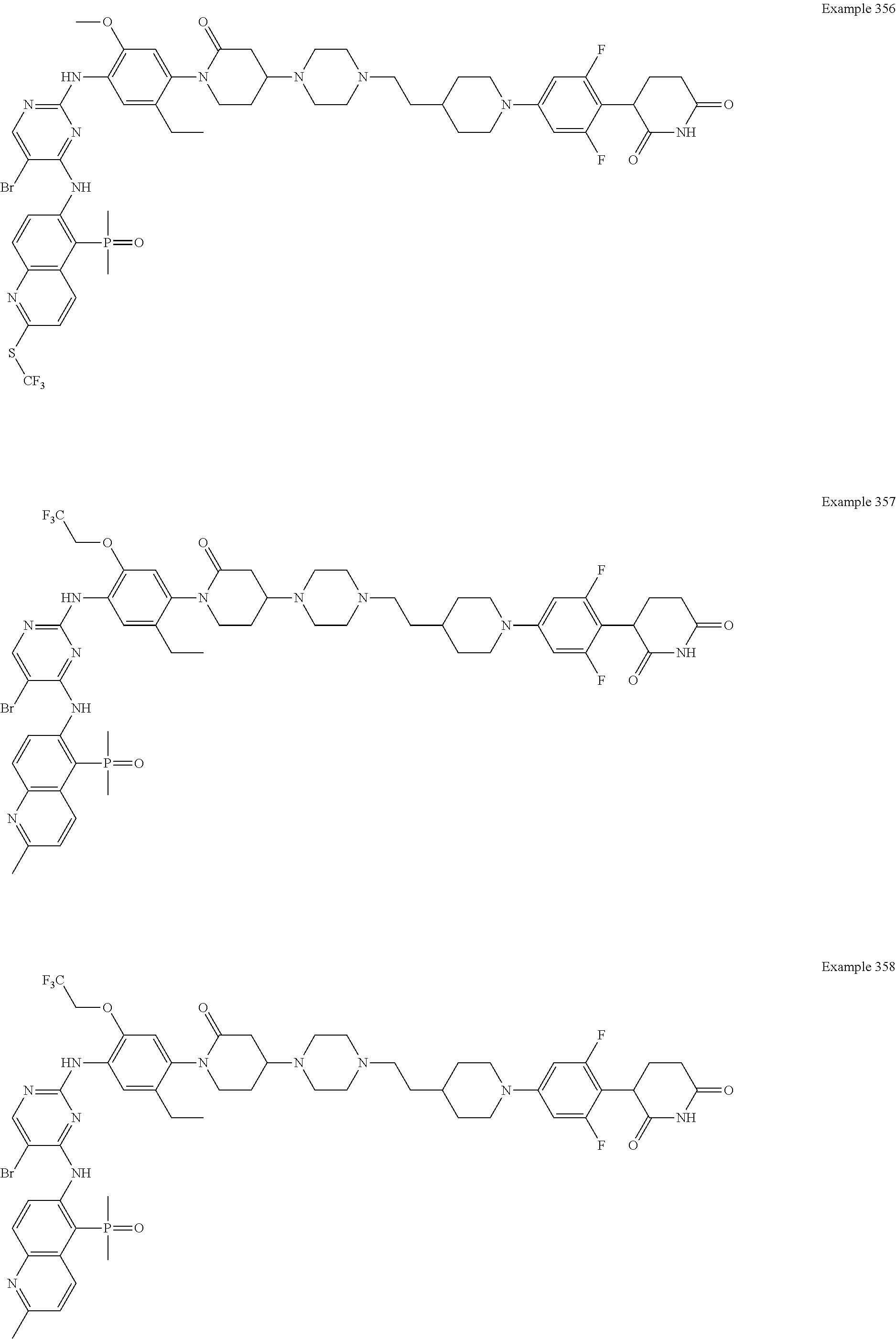 [SMILES: CCc1cc(Nc2ncc(Br)c(Nc3ccc4nc(SC(F)(F)F)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1=O ; CCc1cc(Nc2ncc(Br)c(Nc3ccc4nc(C)ccc4c3P(C)(C)=O)n2)c(OCC(F)(F)F)cc1N1CCC(N2CCN(CCC3CCN(c4cc(F)c(C5CCC(=O)NC5=O)c(F)c4)CC3)CC2)CC1=O ; CCc1cc(Nc2ncc(Br)c(Nc3ccc4nc(C)ccc4c3P(C)(C)=O)n2)c(OCC(F)(F)F)cc1N1CCC(N2CCN(CCC3CCN(c4cc(F)c(C5CCC(=O)NC5=O)c(F)c4)CC3)CC2)CC1=O]

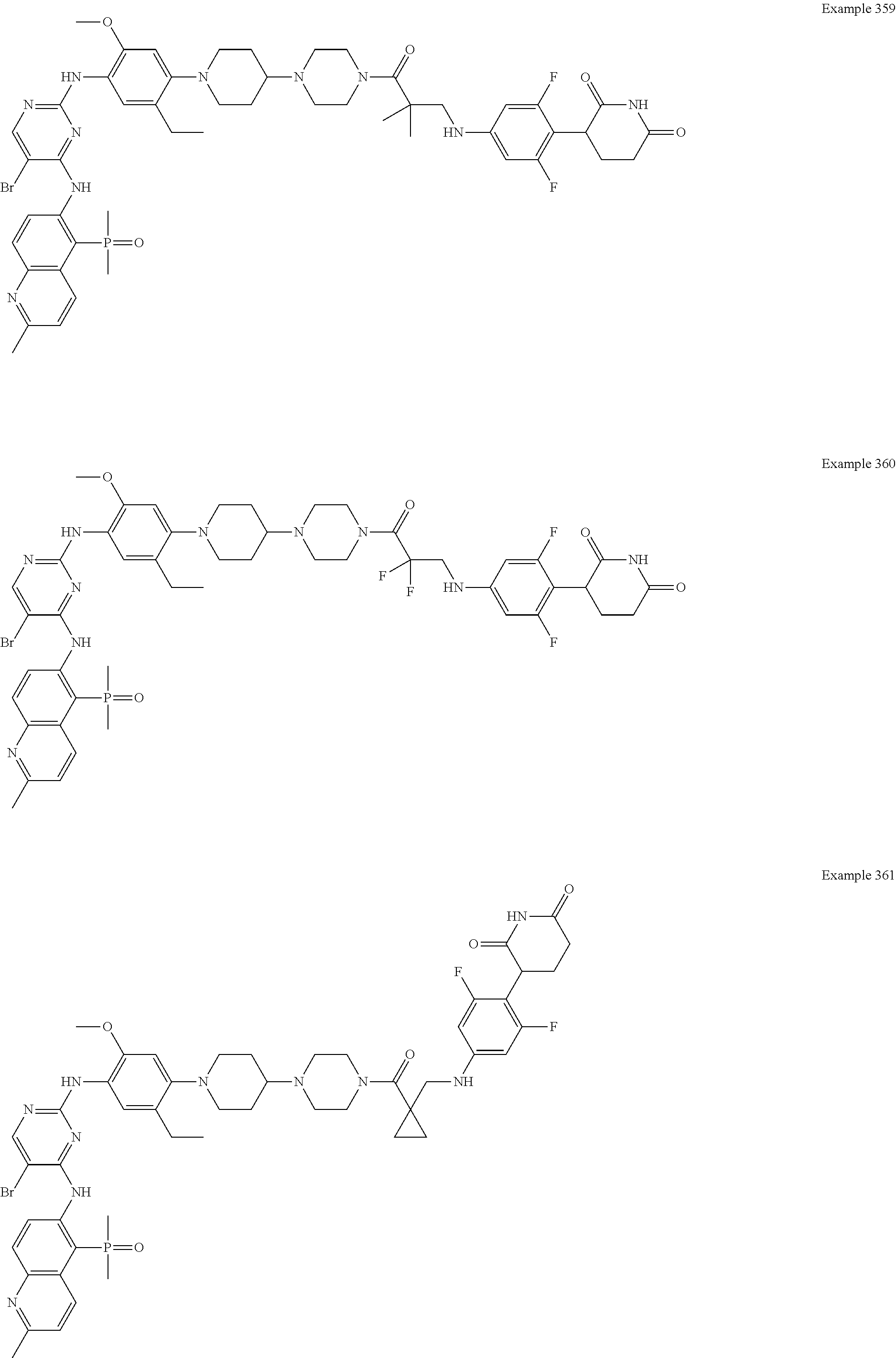 [SMILES: CCc1cc(Nc2ncc(Br)c(Nc3ccc4nc(C)ccc4c3P(C)(C)=O)n2)c(OC)cc1N1CCC(N2CCN(C(=O)C(C)(C)CNc3cc(F)c(C4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C(F)(F)CNc3cc(F)c(C4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3(CNc4cc(F)c(C5CCC(=O)NC5=O)c(F)c4)CC3)CC2)CC1]

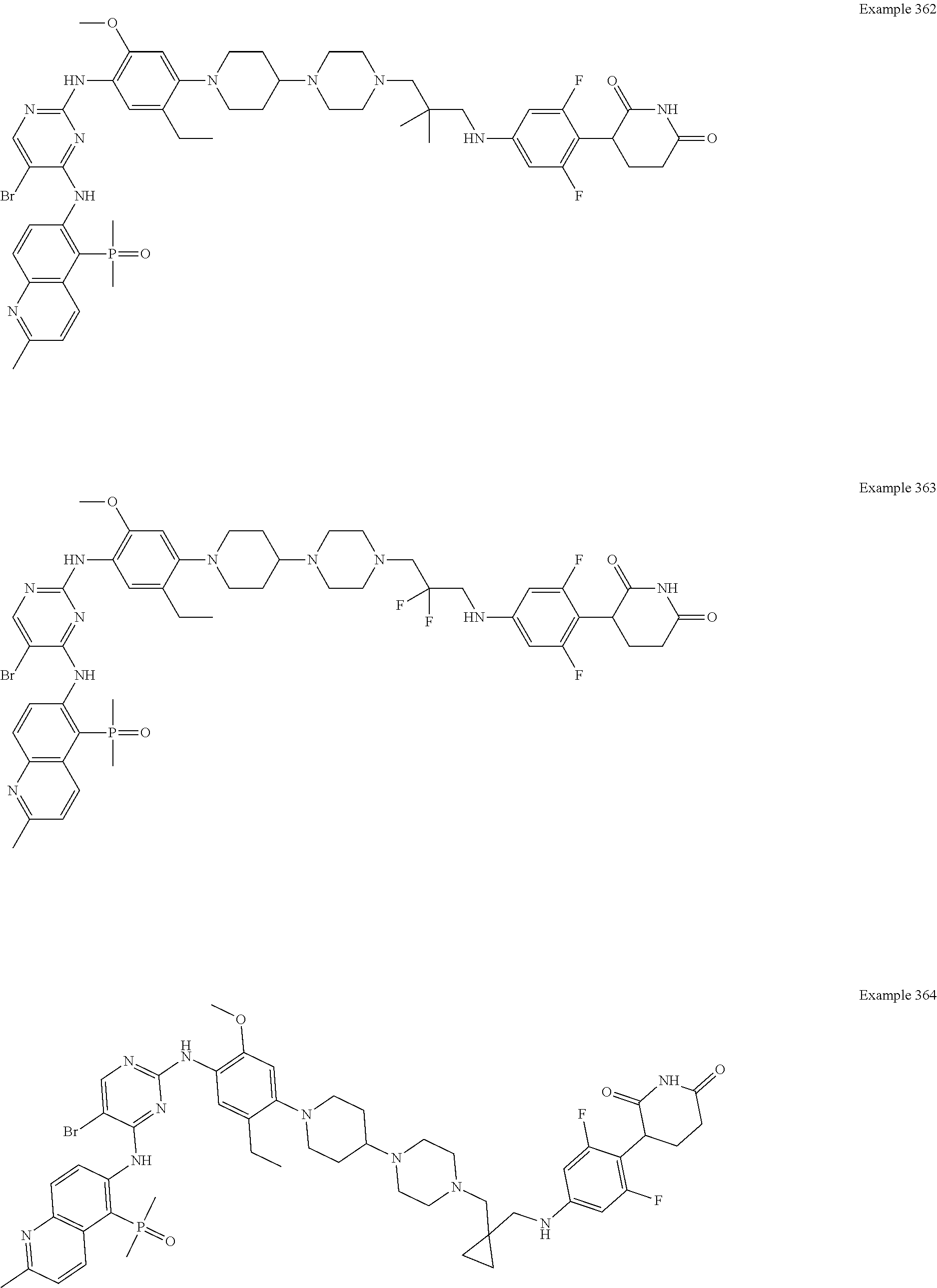 [SMILES: CCc1cc(Nc2ncc(Br)c(Nc3ccc4nc(C)ccc4c3P(C)(C)=O)n2)c(OC)cc1N1CCC(N2CCN(CC(C)(C)CNc3cc(F)c(C4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CCN(CC(F)(F)CNc3cc(F)c(C4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CCN(CC3(CNc4cc(F)c(C5CCC(=O)NC5=O)c(F)c4)CC3)CC2)CC1]

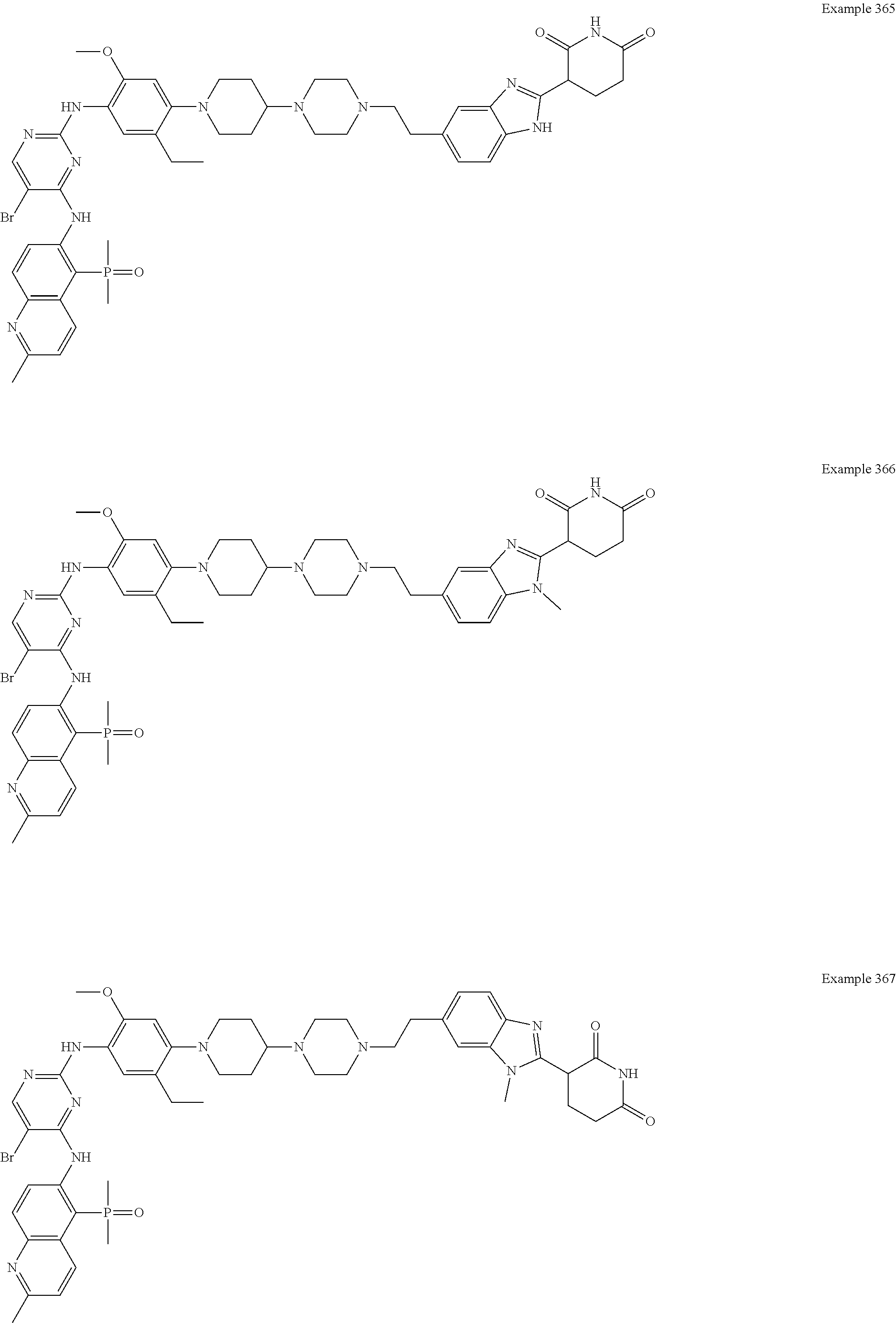 [SMILES: CCc1cc(Nc2ncc(Br)c(Nc3ccc4nc(C)ccc4c3P(C)(C)=O)n2)c(OC)cc1N1CCC(N2CCN(CCc3ccc4[nH]c(C5CCC(=O)NC5=O)nc4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)nc(C3CCC(=O)NC3=O)n4C)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4nc(C5CCC(=O)NC5=O)n(C)c4c3)CC2)CC1]

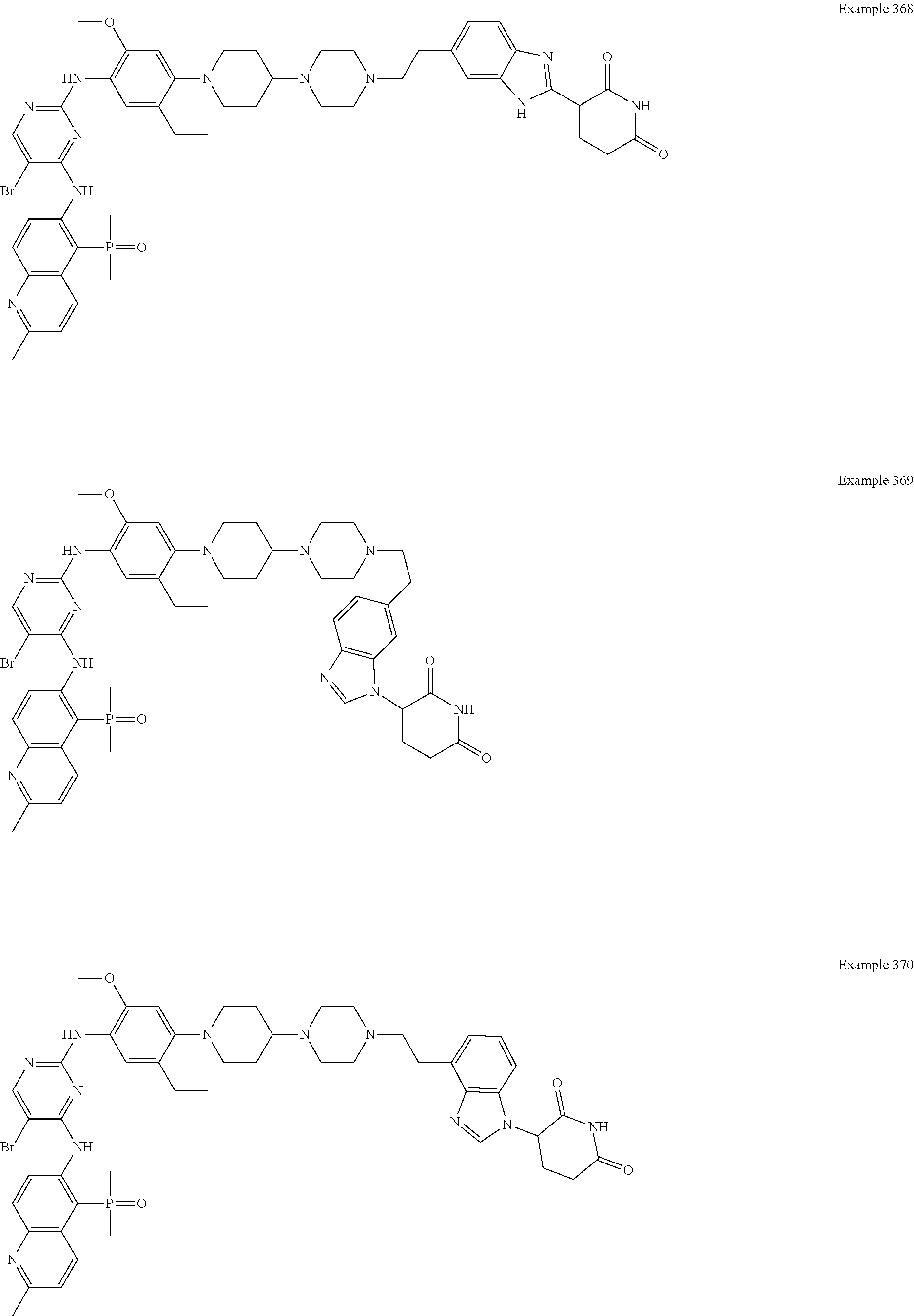 [SMILES: CCc1cc(Nc2ncc(Br)c(Nc3ccc4nc(C)ccc4c3P(C)(C)=O)n2)c(OC)cc1N1CCC(N2CCN(CCc3ccc4nc(C5CCC(=O)NC5=O)[nH]c4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4ncn(C5CCC(=O)NC5=O)c4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3cccc4c3ncn4C3CCC(=O)NC3=O)CC2)CC1]

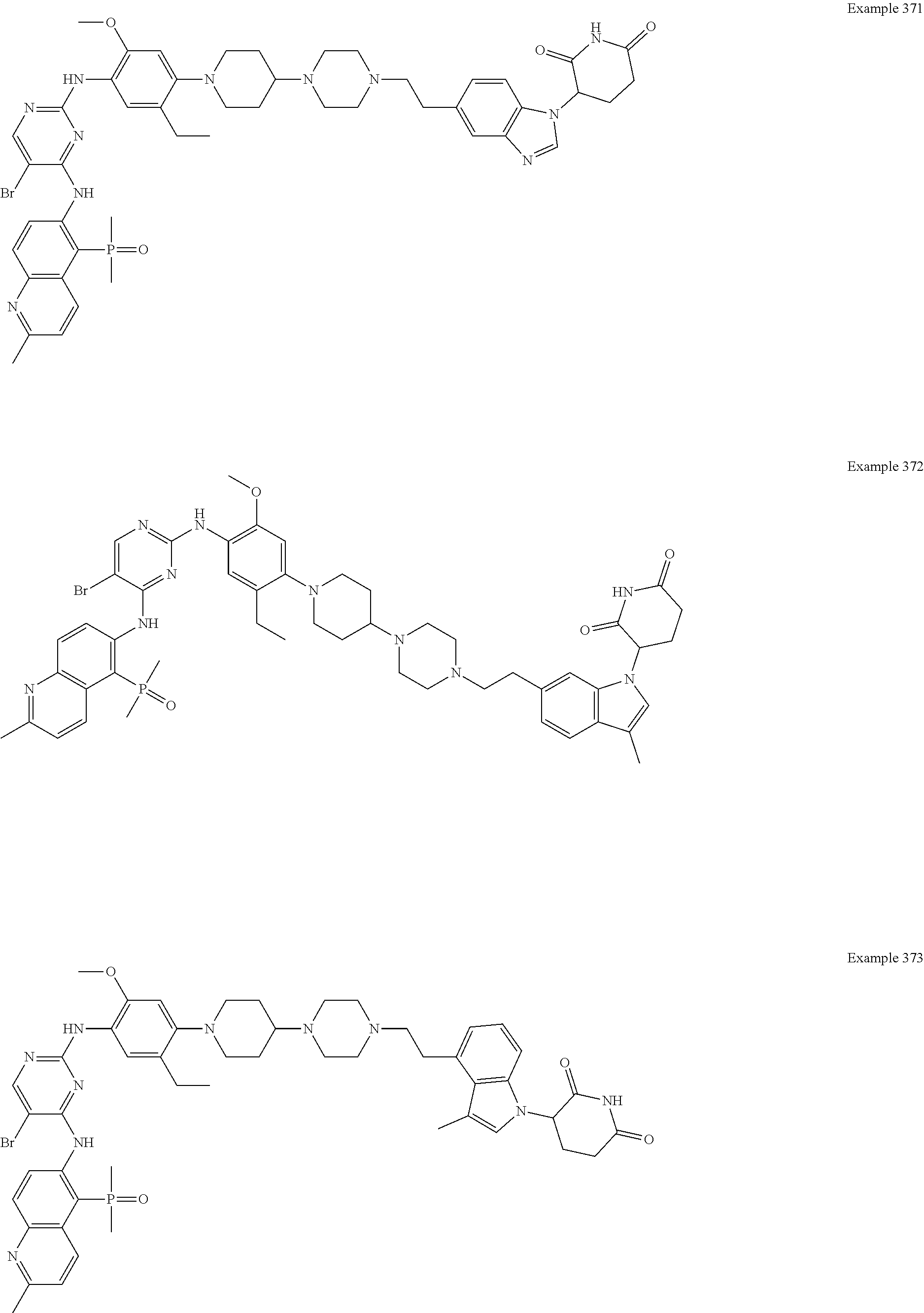 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)ncn4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(C)cn(C5CCC(=O)NC5=O)c4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3cccc4c3c(C)cn4C3CCC(=O)NC3=O)CC2)CC1]

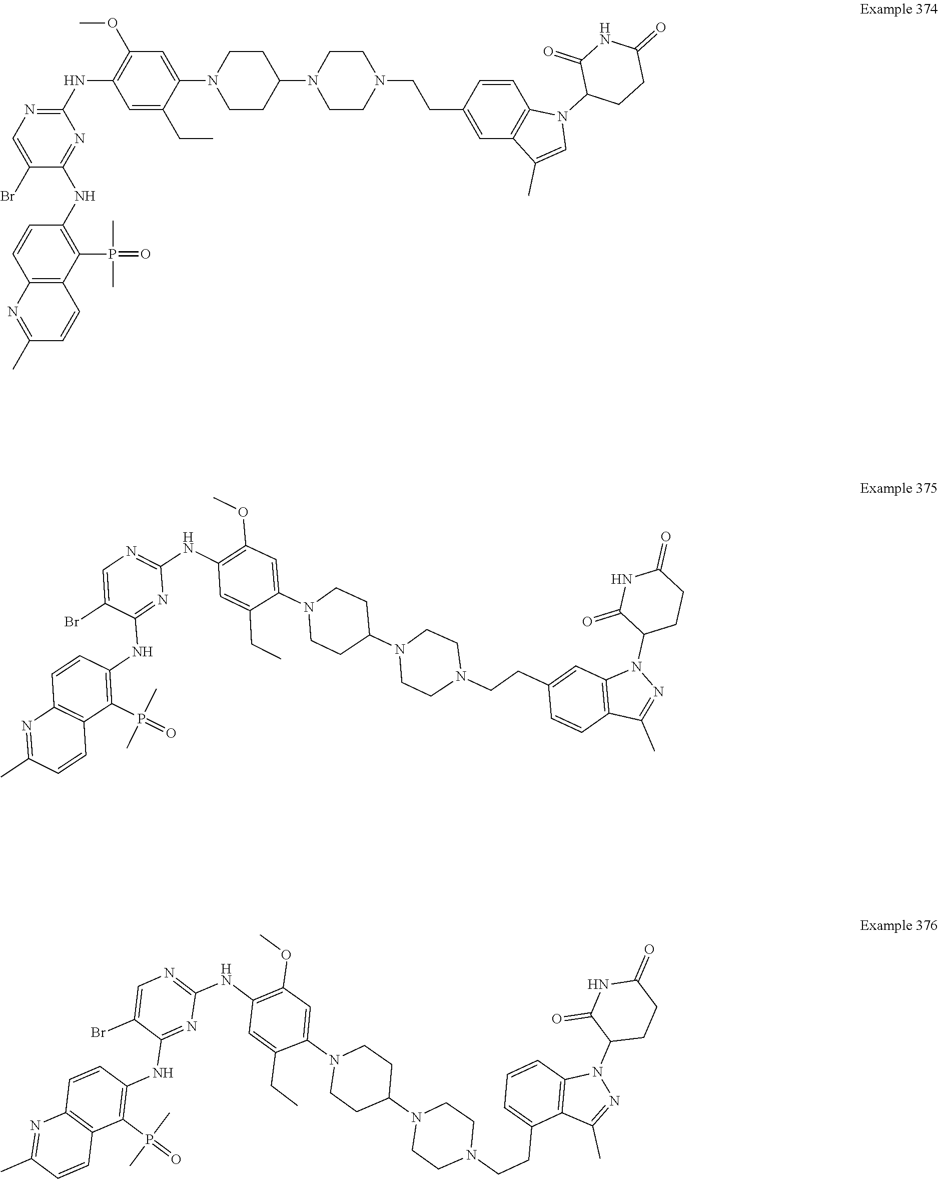 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)c(C)cn4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(C)nn(C5CCC(=O)NC5=O)c4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3cccc4c3c(C)nn4C3CCC(=O)NC3=O)CC2)CC1]

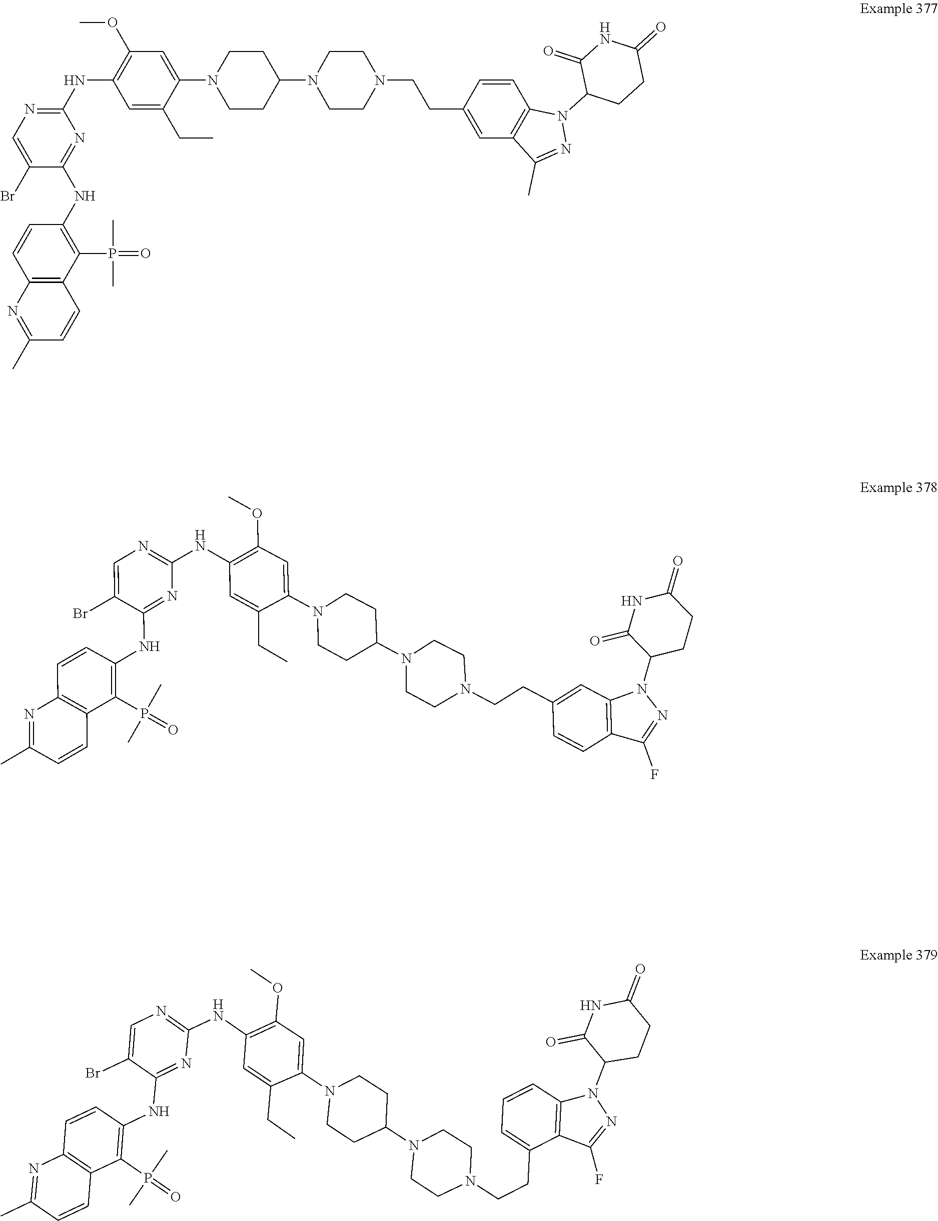 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)c(C)nn4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(F)nn(C5CCC(=O)NC5=O)c4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3cccc4c3c(F)nn4C3CCC(=O)NC3=O)CC2)CC1]

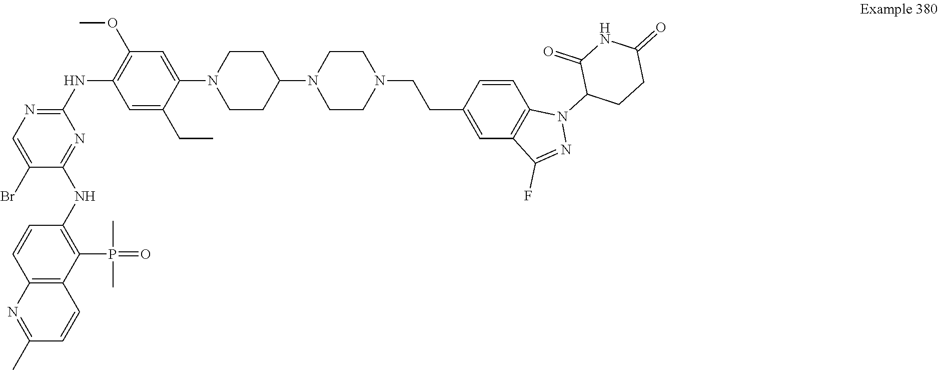 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)c(F)nn4C3CCC(=O)NC3=O)CC2)CC1]

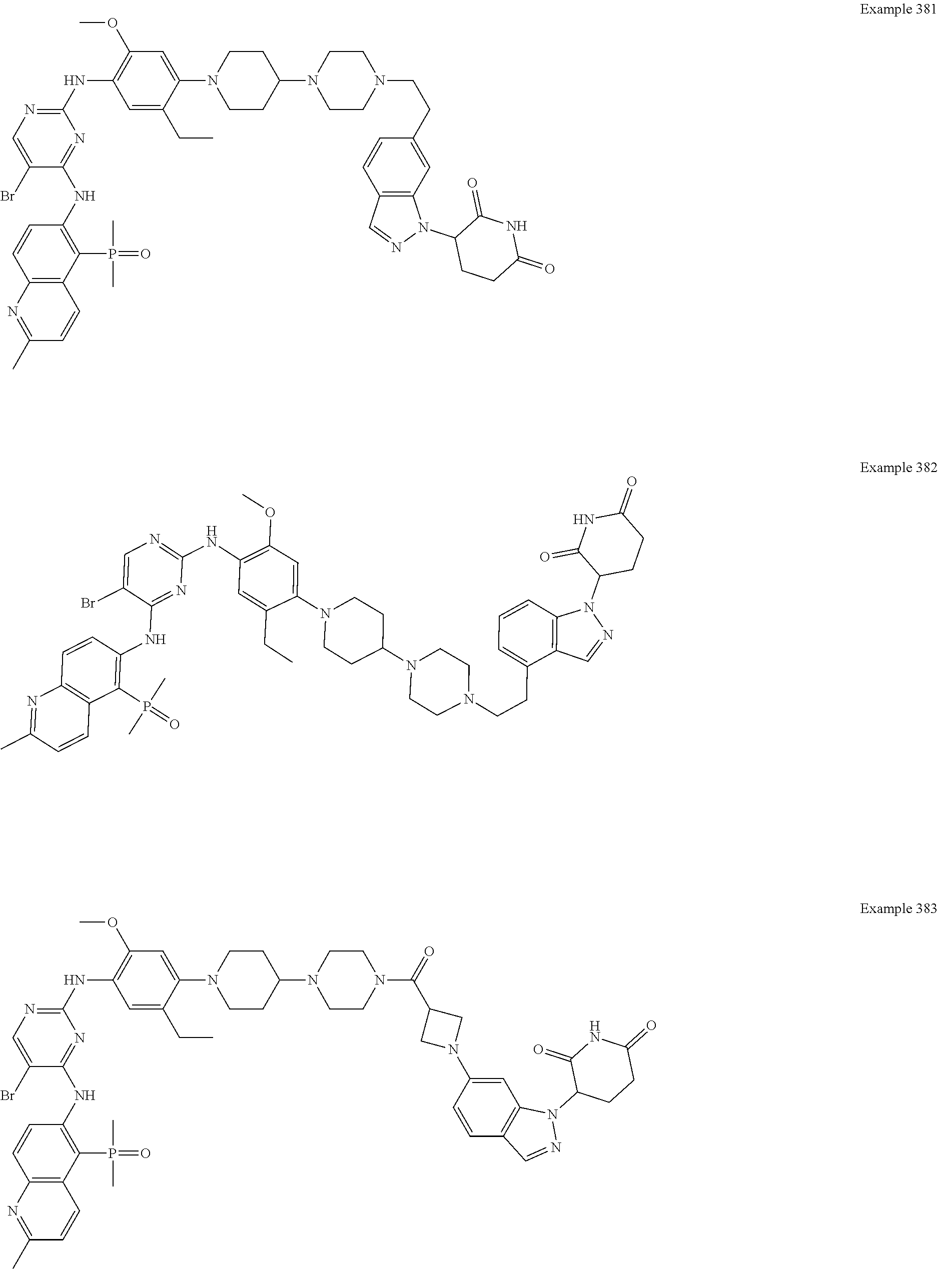 [SMILES: CCc1cc(Nc2ncc(Br)c(Nc3ccc4nc(C)ccc4c3P(C)(C)=O)n2)c(OC)cc1N1CCC(N2CCN(CCc3ccc4cnn(C5CCC(=O)NC5=O)c4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3cccc4c3cnn4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CN(c4ccc5cnn(C6CCC(=O)NC6=O)c5c4)C3)CC2)CC1]

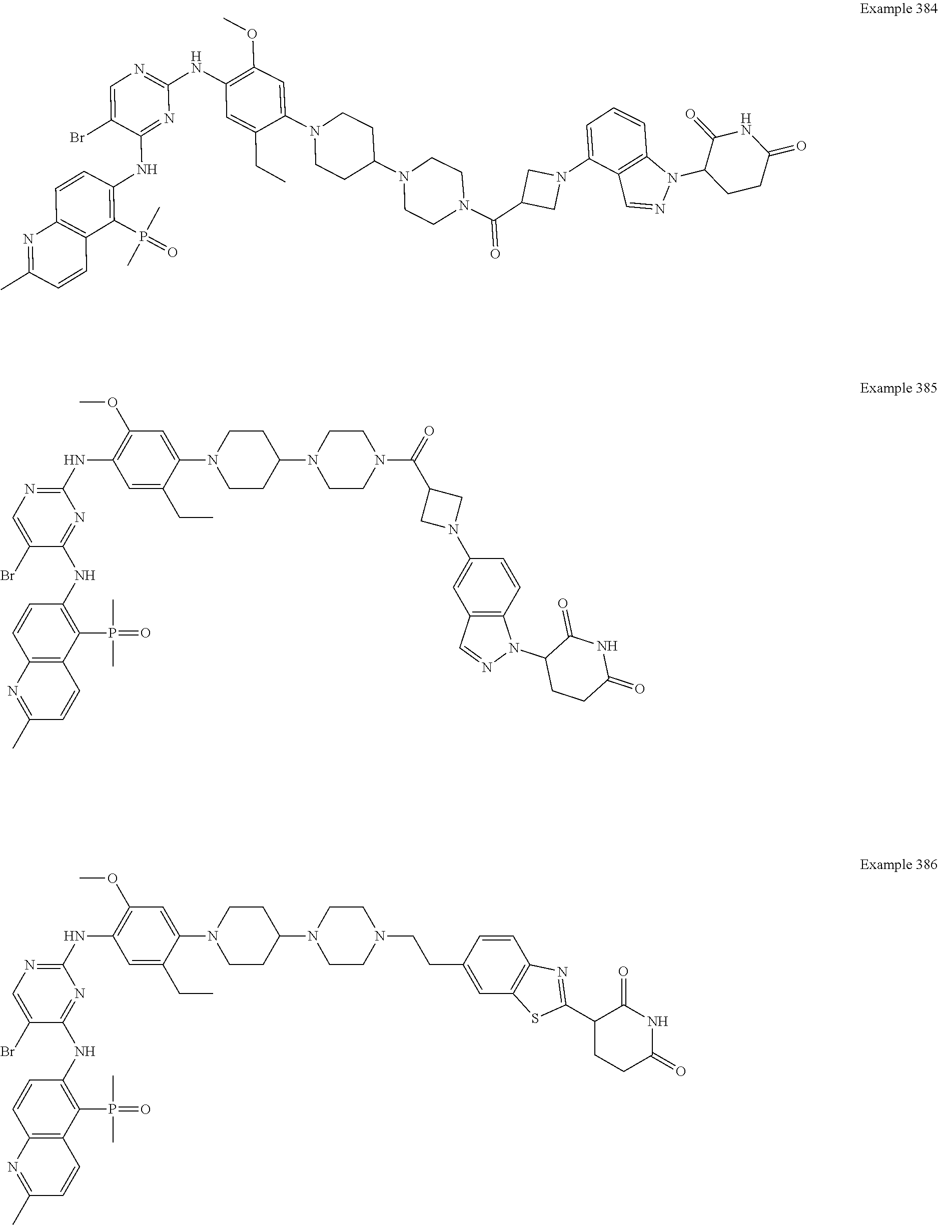 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4cnn5C4CCC(=O)NC4=O)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CN(c4ccc5c(cnn5C5CCC(=O)NC5=O)c4)C3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4nc(C5CCC(=O)NC5=O)sc4c3)CC2)CC1]

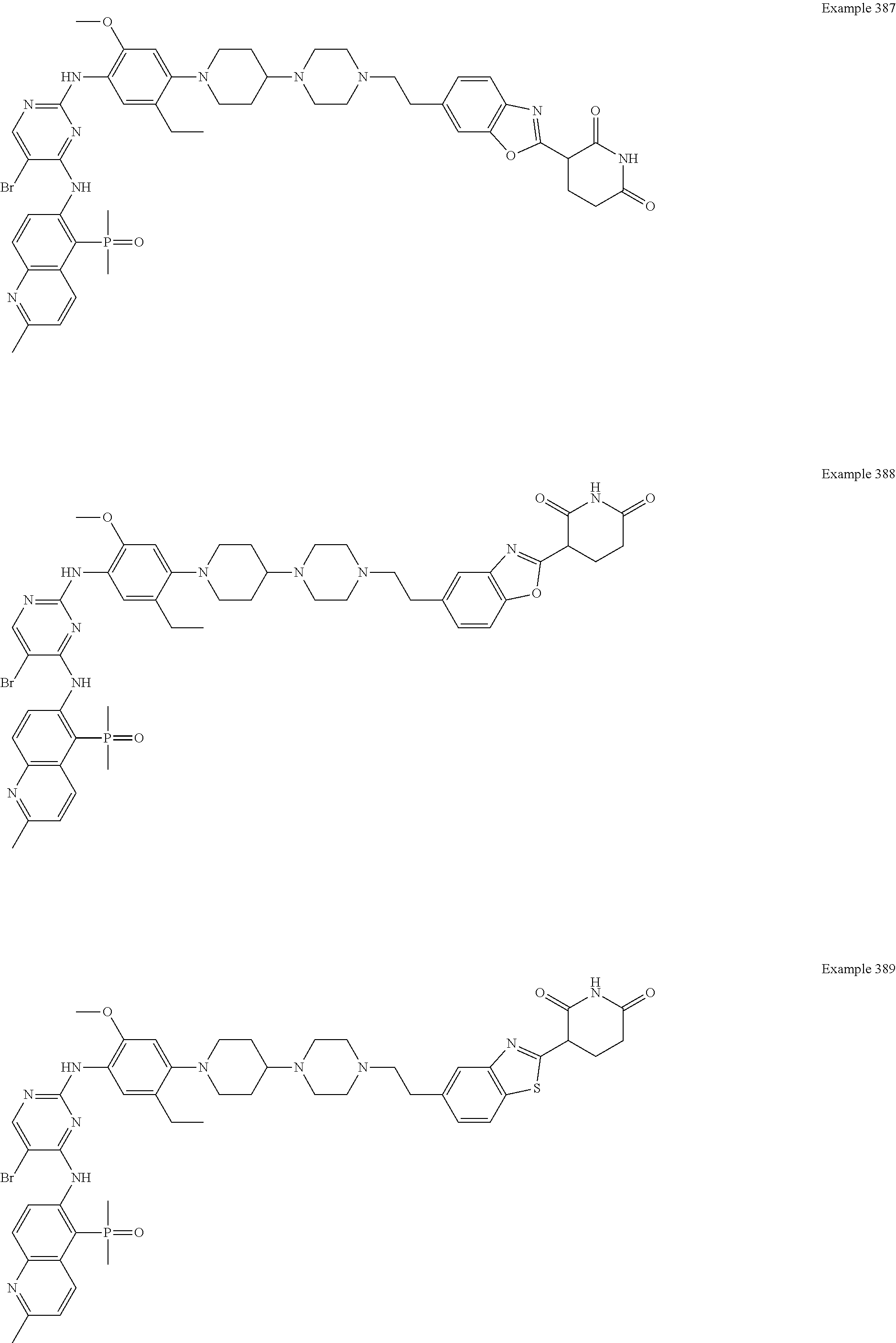 [SMILES: CCc1cc(Nc2ncc(Br)c(Nc3ccc4nc(C)ccc4c3P(C)(C)=O)n2)c(OC)cc1N1CCC(N2CCN(CCc3ccc4nc(C5CCC(=O)NC5=O)oc4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4oc(C5CCC(=O)NC5=O)nc4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4sc(C5CCC(=O)NC5=O)nc4c3)CC2)CC1]

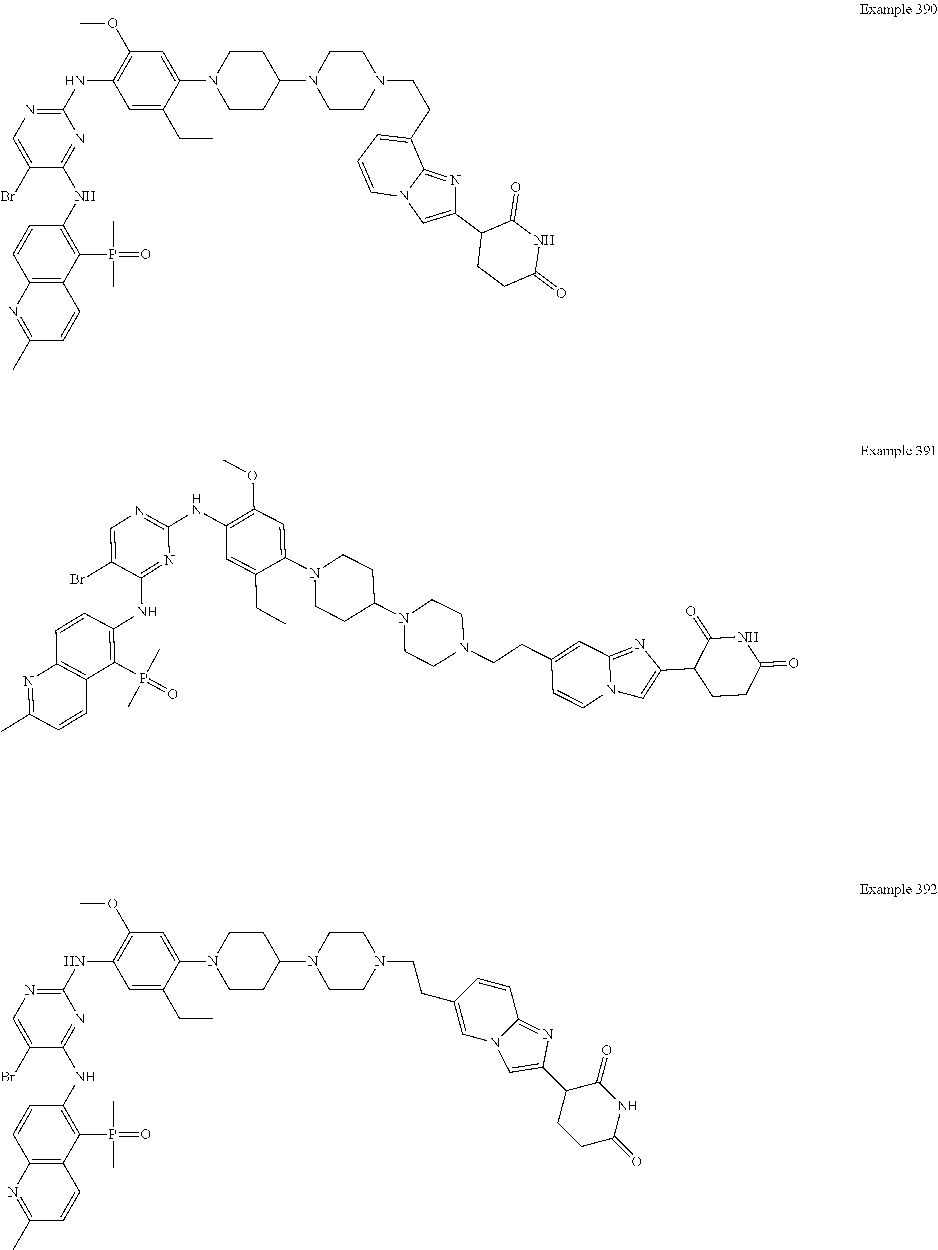 [SMILES: CCc1cc(Nc2ncc(Br)c(Nc3ccc4nc(C)ccc4c3P(C)(C)=O)n2)c(OC)cc1N1CCC(N2CCN(CCc3cccn4cc(C5CCC(=O)NC5=O)nc34)CC2)CC1 ; CCc1cc(Nc2ncc(Br)c(Nc3ccc4nc(C)ccc4c3P(C)(C)=O)n2)c(OC)cc1N1CCC(N2CCN(CCc3ccn4cc(C5CCC(=O)NC5=O)nc4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4nc(C5CCC(=O)NC5=O)cn4c3)CC2)CC1]

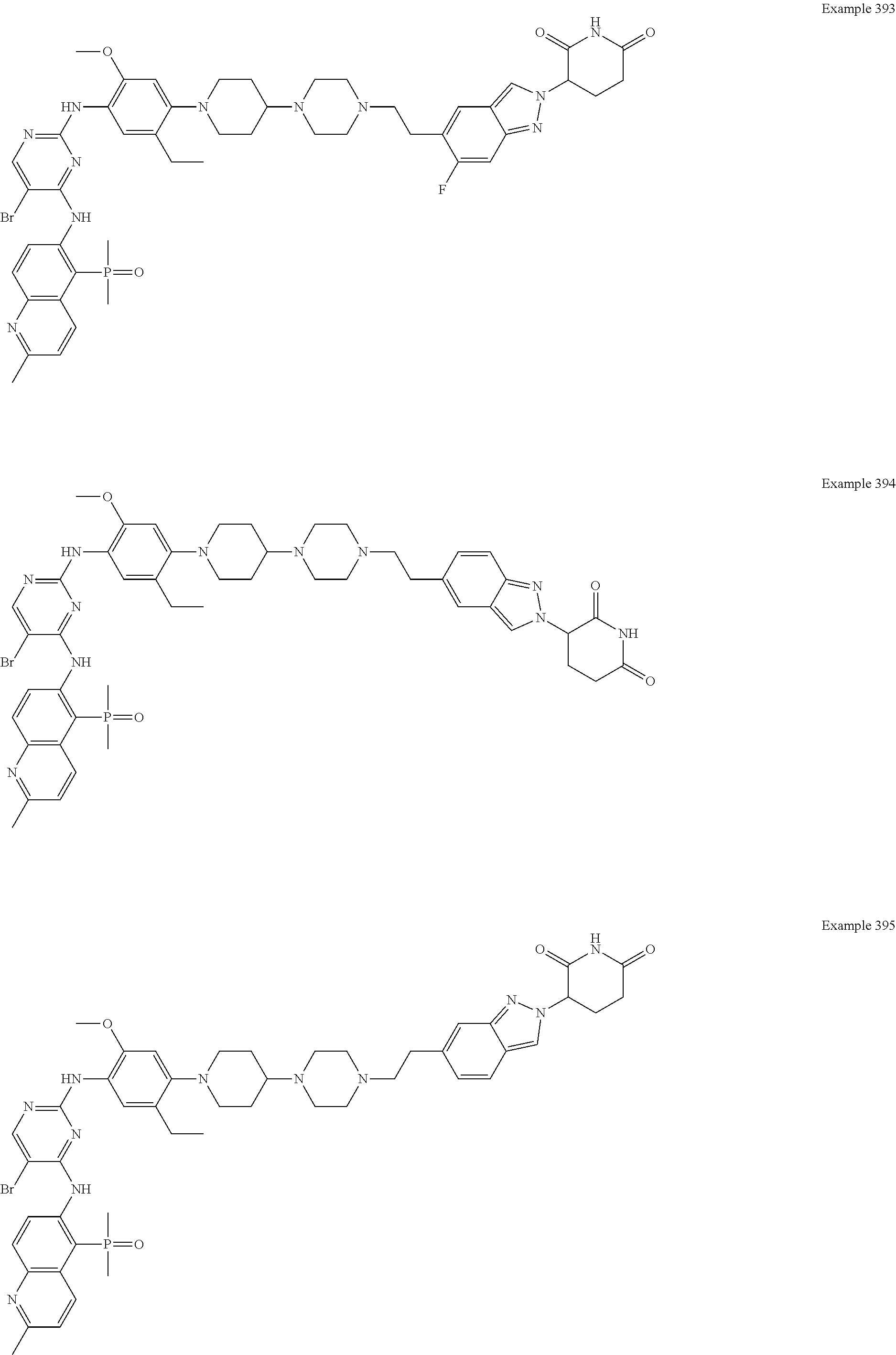 [SMILES: CCc1cc(Nc2ncc(Br)c(Nc3ccc4nc(C)ccc4c3P(C)(C)=O)n2)c(OC)cc1N1CCC(N2CCN(CCc3cc4cn(C5CCC(=O)NC5=O)nc4cc3F)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4nn(C5CCC(=O)NC5=O)cc4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4cn(C5CCC(=O)NC5=O)nc4c3)CC2)CC1]

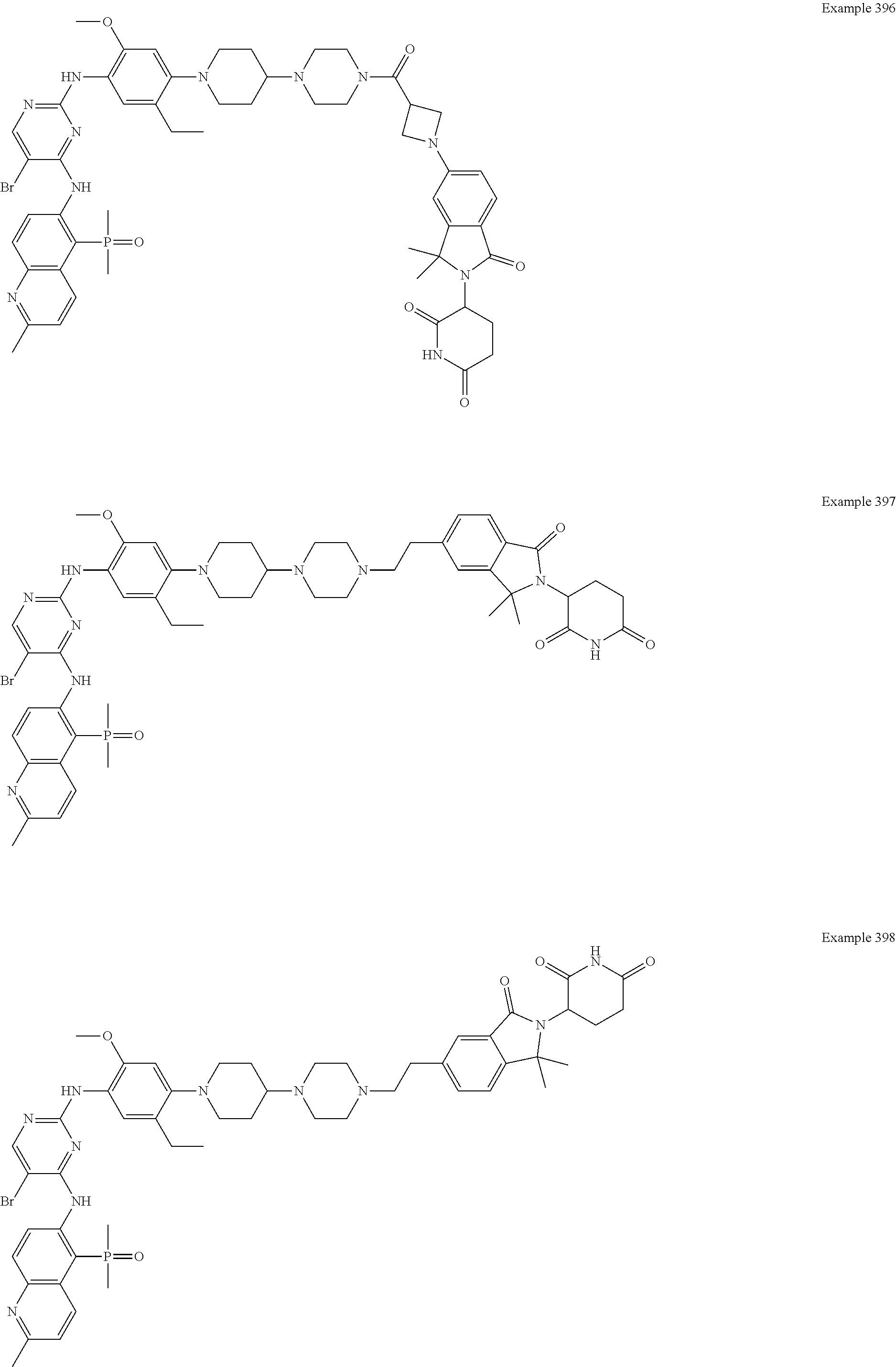 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4ccc5c(c4)C(C)(C)N(C4CCC(=O)NC4=O)C5=O)C3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)C(C)(C)N(C3CCC(=O)NC3=O)C4=O)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)C(=O)N(C3CCC(=O)NC3=O)C4(C)C)CC2)CC1]

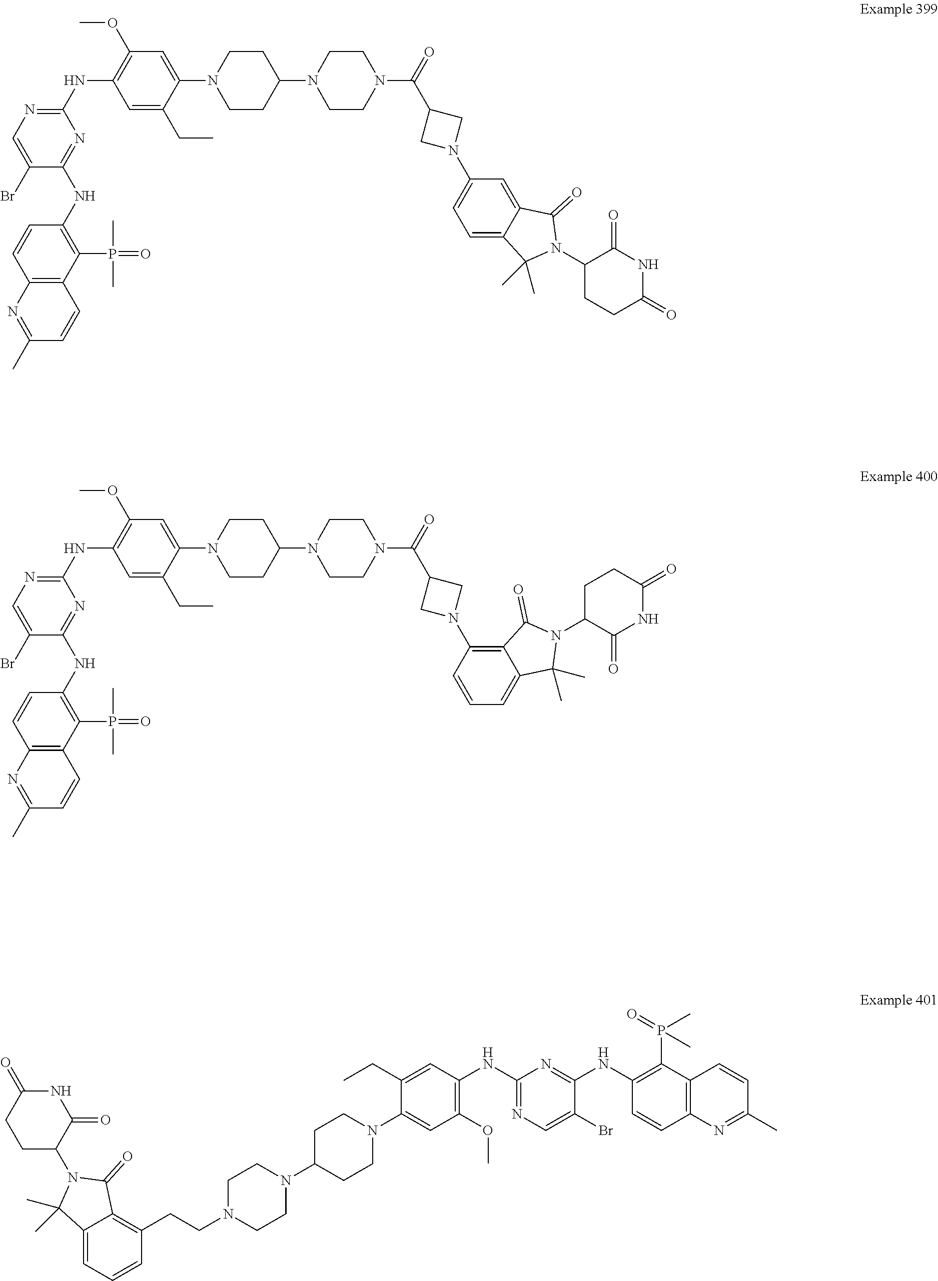 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4ccc5c(c4)C(=O)N(C4CCC(=O)NC4=O)C5(C)C)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4C(=O)N(C4CCC(=O)NC4=O)C5(C)C)C3)CC2)CC1 ; CCc1cc(Nc2ncc(Br)c(Nc3ccc4nc(C)ccc4c3P(C)(C)=O)n2)c(OC)cc1N1CCC(N2CCN(CCc3cccc4c3C(=O)N(C3CCC(=O)NC3=O)C4(C)C)CC2)CC1]

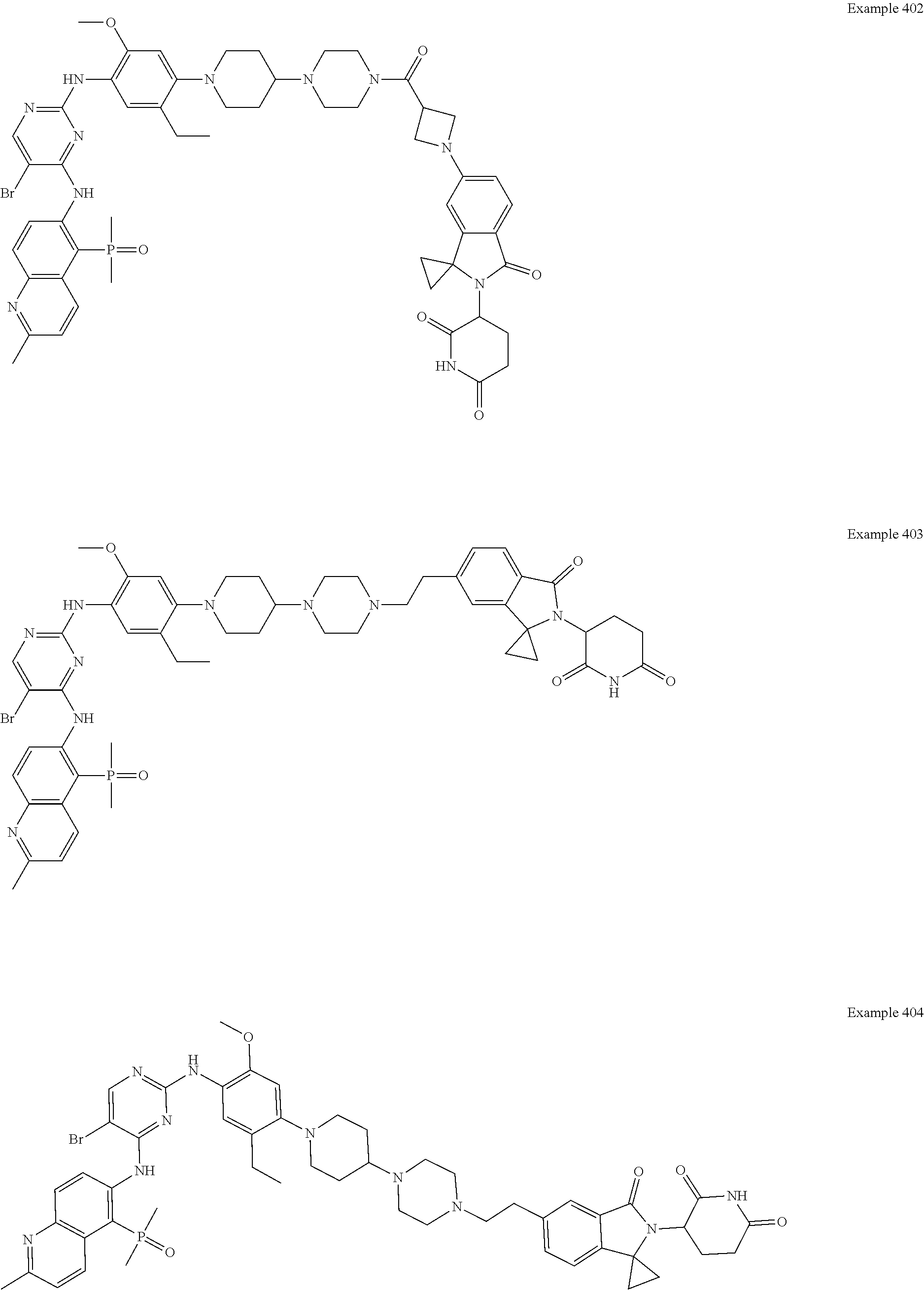 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4ccc5c(c4)C4(CC4)N(C4CCC(=O)NC4=O)C5=O)C3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)C3(CC3)N(C3CCC(=O)NC3=O)C4=O)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)C(=O)N(C3CCC(=O)NC3=O)C43CC3)CC2)CC1]

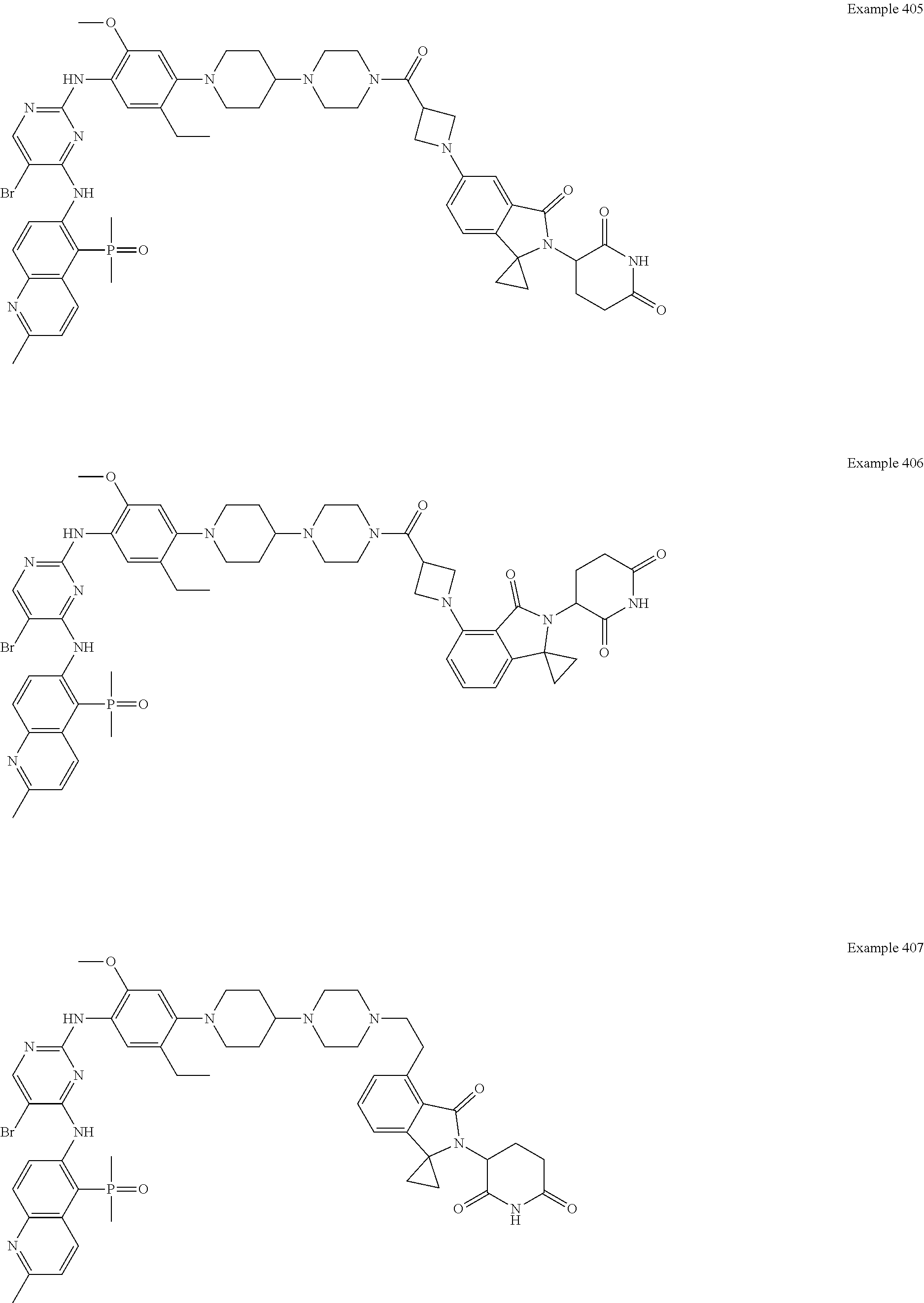 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4ccc5c(c4)C(=O)N(C4CCC(=O)NC4=O)C54CC4)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4C(=O)N(C4CCC(=O)NC4=O)C54CC4)C3)CC2)CC1 ; CCc1cc(Nc2ncc(Br)c(Nc3ccc4nc(C)ccc4c3P(C)(C)=O)n2)c(OC)cc1N1CCC(N2CCN(CCc3cccc4c3C(=O)N(C3CCC(=O)NC3=O)C43CC3)CC2)CC1]

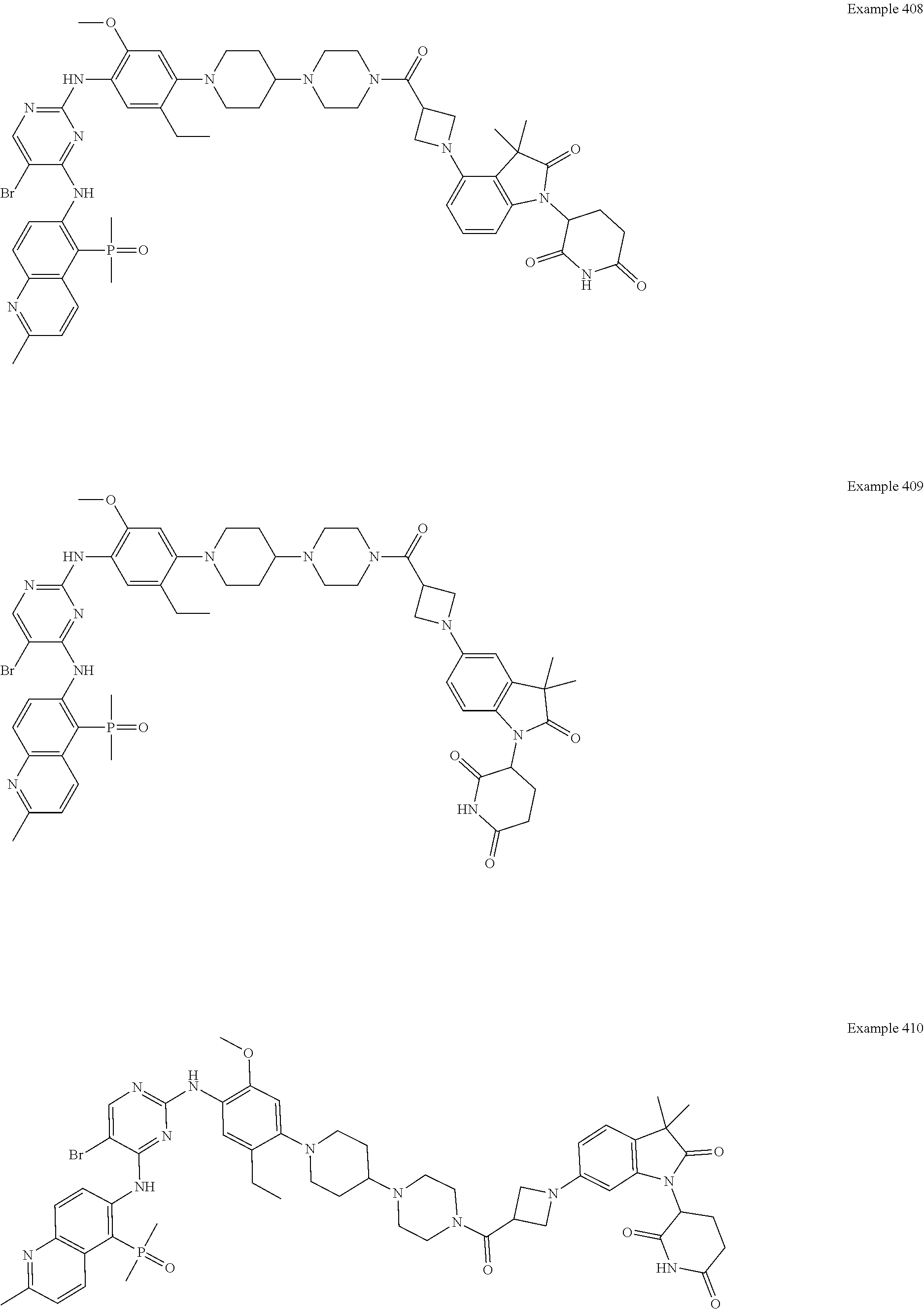 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4C(C)(C)C(=O)N5C4CCC(=O)NC4=O)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CN(c4ccc5c(c4)C(C)(C)C(=O)N5C4CCC(=O)NC4=O)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CN(c4ccc5c(c4)N(C4CCC(=O)NC4=O)C(=O)C5(C)C)C3)CC2)CC1]

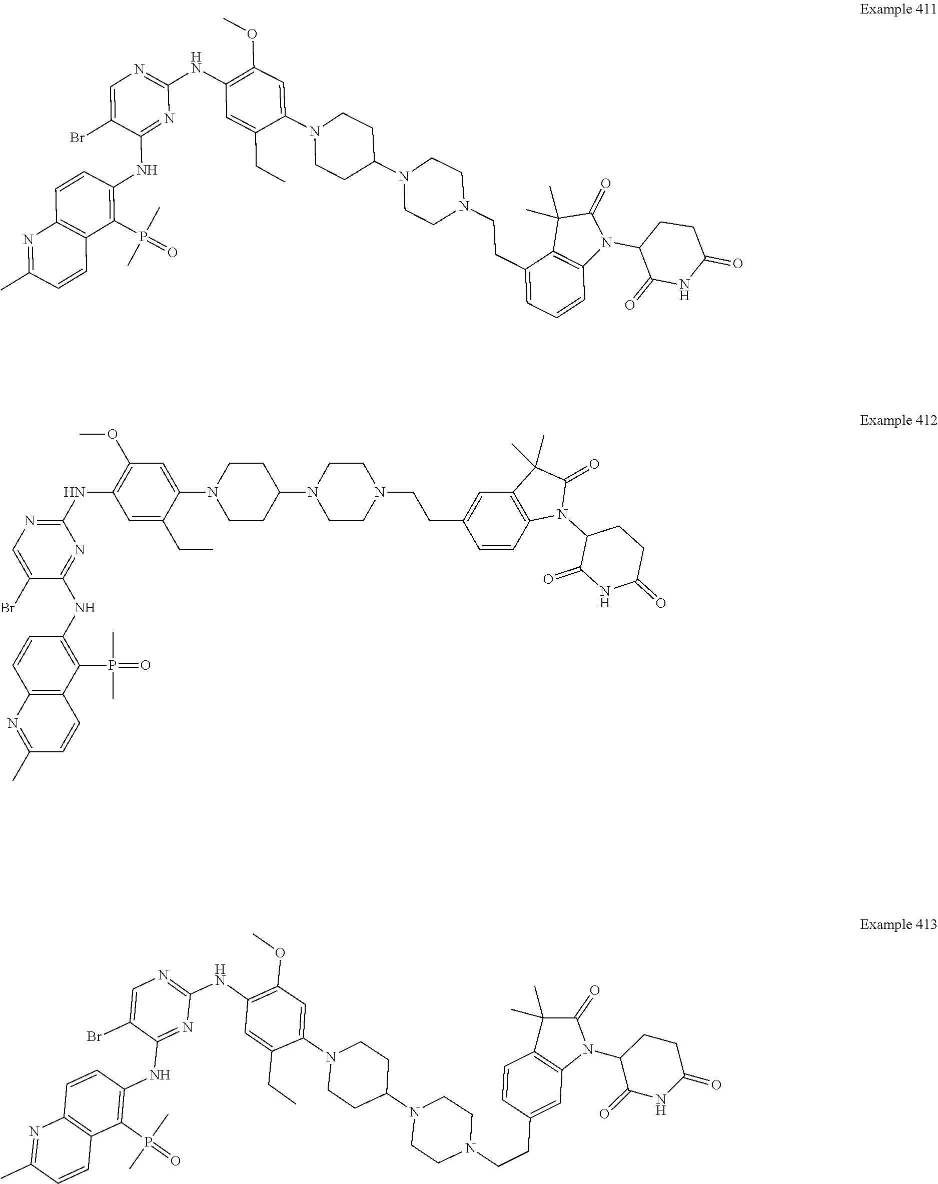 [SMILES: CCc1cc(Nc2ncc(Br)c(Nc3ccc4nc(C)ccc4c3P(C)(C)=O)n2)c(OC)cc1N1CCC(N2CCN(CCc3cccc4c3C(C)(C)C(=O)N4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)C(C)(C)C(=O)N4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)N(C3CCC(=O)NC3=O)C(=O)C4(C)C)CC2)CC1]

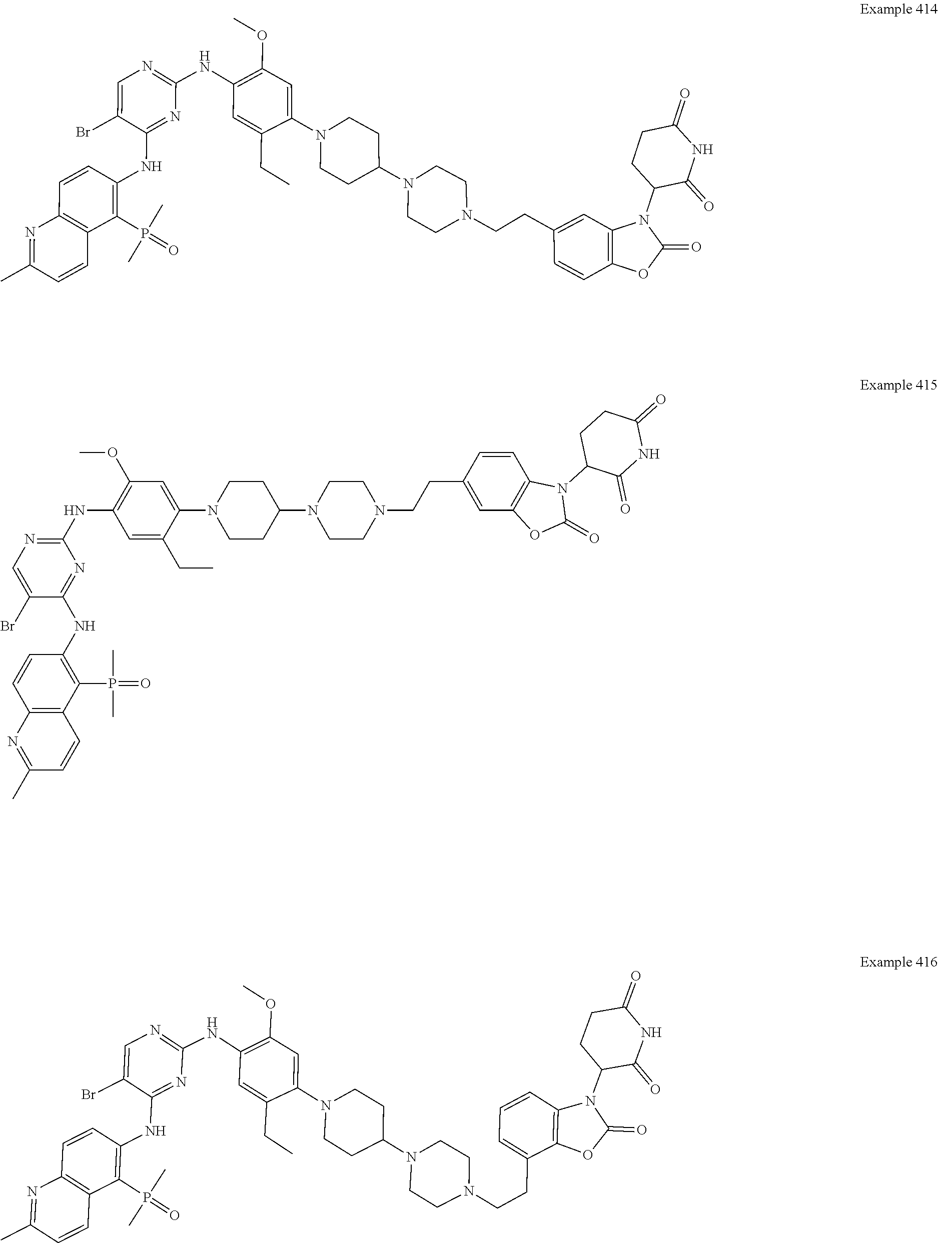 [SMILES: CCc1cc(Nc2ncc(Br)c(Nc3ccc4nc(C)ccc4c3P(C)(C)=O)n2)c(OC)cc1N1CCC(N2CCN(CCc3ccc4oc(=O)n(C5CCC(=O)NC5=O)c4c3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)oc(=O)n4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(CCc3cccc4c3oc(=O)n4C3CCC(=O)NC3=O)CC2)CC1]

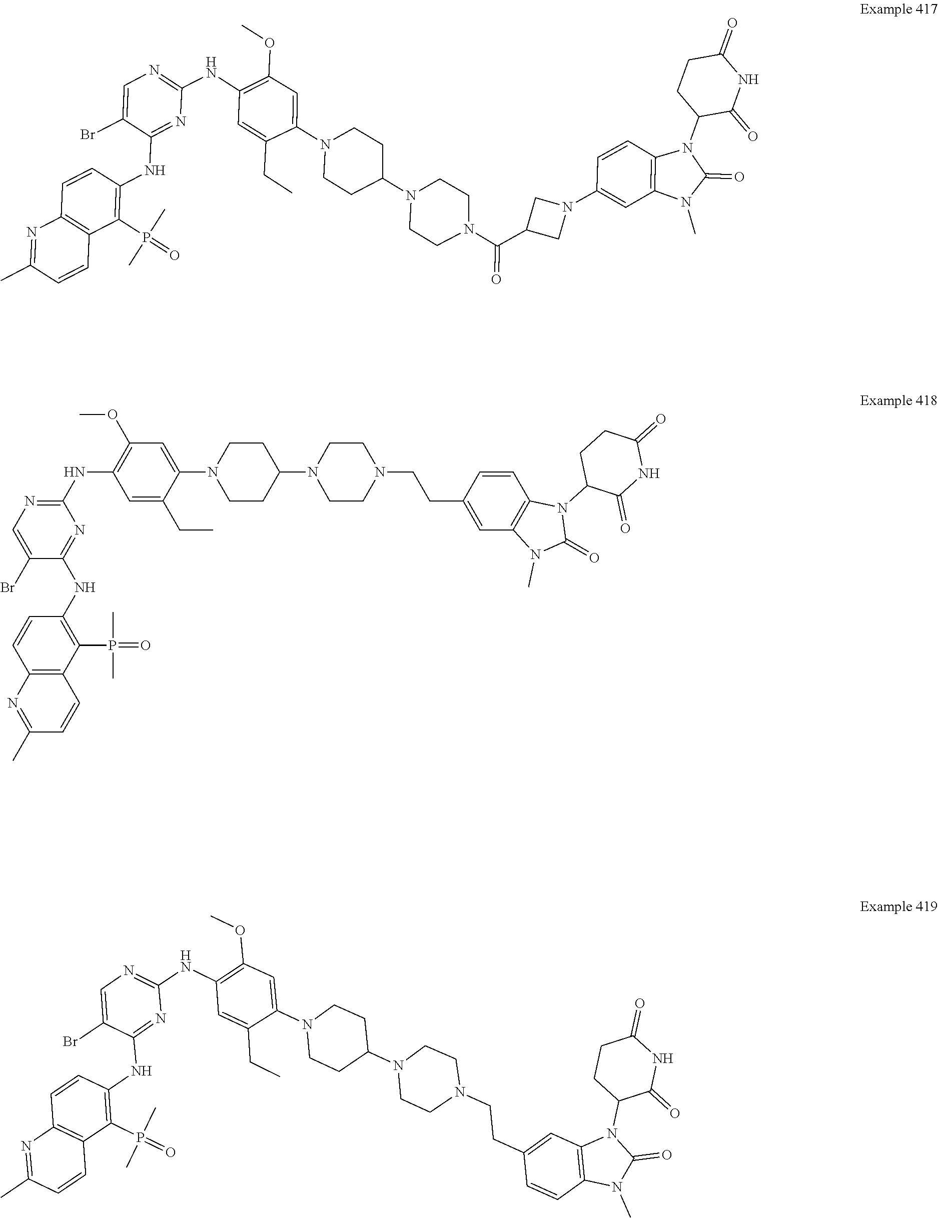 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4ccc5c(c4)n(C)c(=O)n5C4CCC(=O)NC4=O)C3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)n(C)c(=O)n4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)n(C3CCC(=O)NC3=O)c(=O)n4C)CC2)CC1]

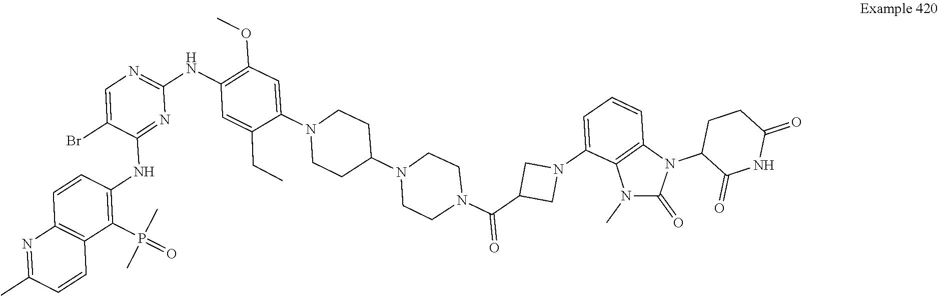 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4n(C)c(=O)n5C4CCC(=O)NC4=O)C3)CC2)CC1]

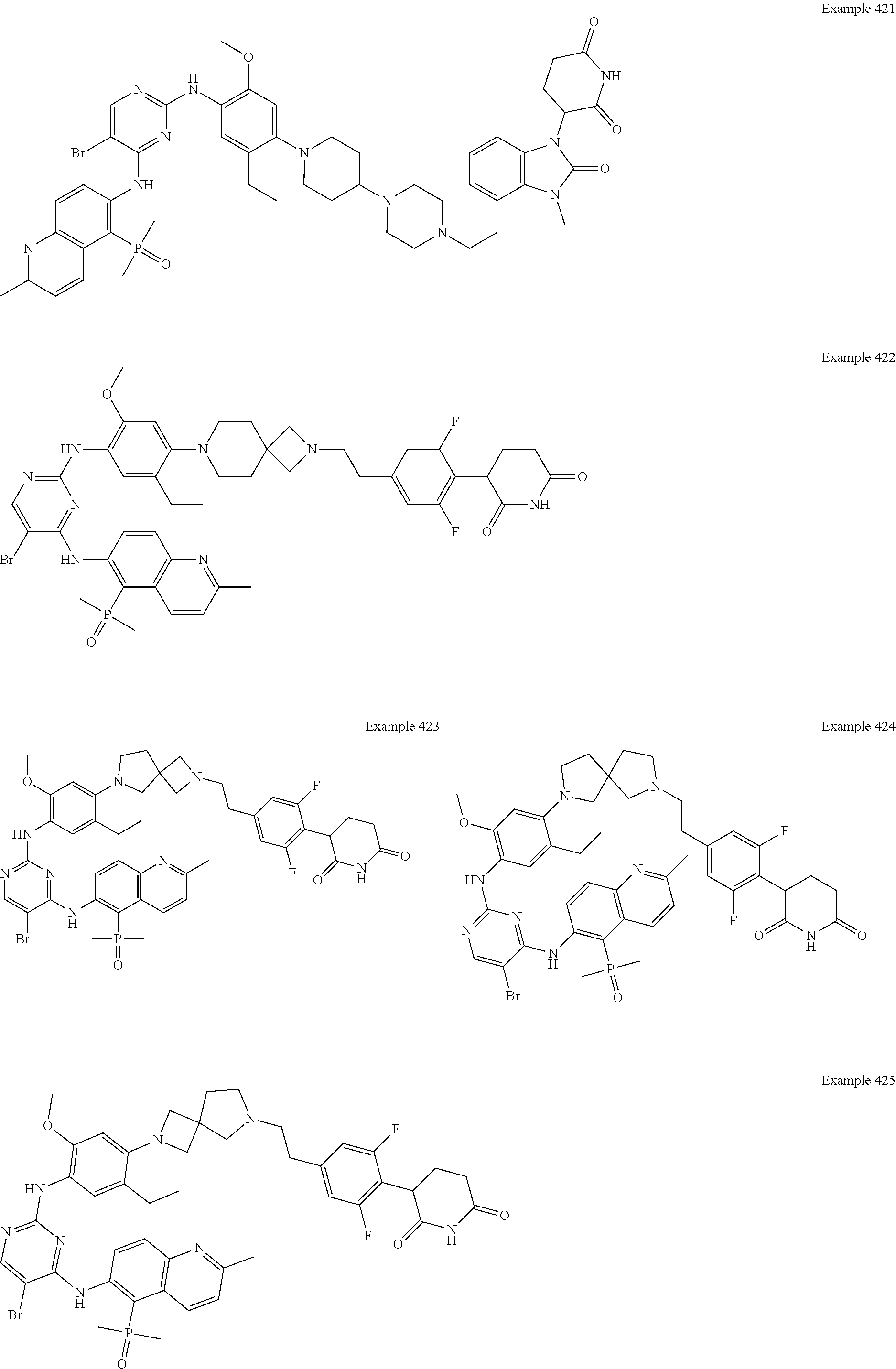

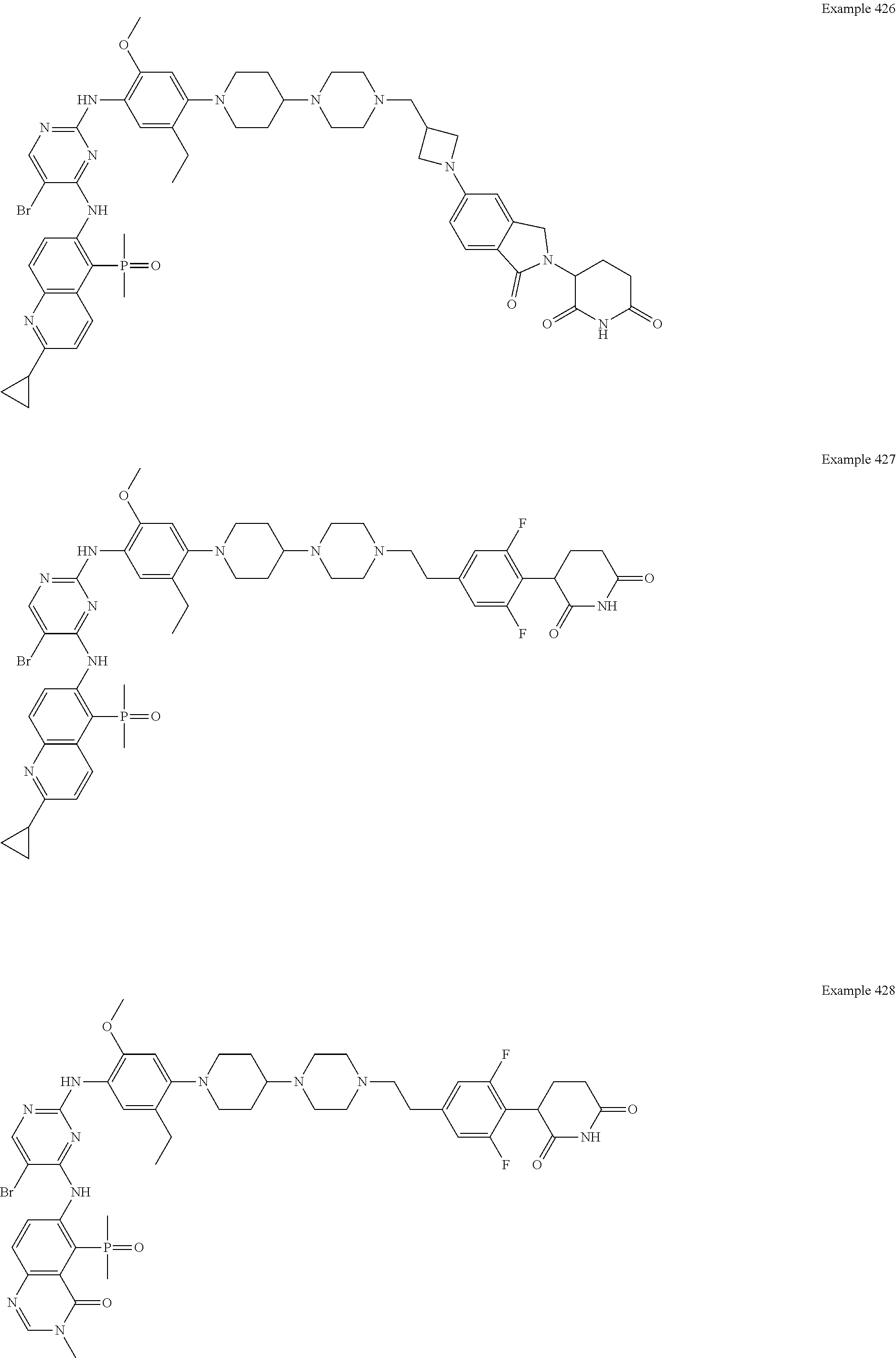 [SMILES: CCc1cc(Nc2ncc(Br)c(Nc3ccc4nc(C5CC5)ccc4c3P(C)(C)=O)n2)c(OC)cc1N1CCC(N2CCN(CC3CN(c4ccc5c(c4)CN(C4CCC(=O)NC4=O)C5=O)C3)CC2)CC1 ; CCc1cc(Nc2ncc(Br)c(Nc3ccc4nc(C5CC5)ccc4c3P(C)(C)=O)n2)c(OC)cc1N1CCC(N2CCN(CCc3cc(F)c(C4CCC(=O)NC4=O)c(F)c3)CC2)CC1 ; CCc1cc(Nc2ncc(Br)c(Nc3ccc4ncn(C)c(=O)c4c3P(C)(C)=O)n2)c(OC)cc1N1CCC(N2CCN(CCc3cc(F)c(C4CCC(=O)NC4=O)c(F)c3)CC2)CC1]

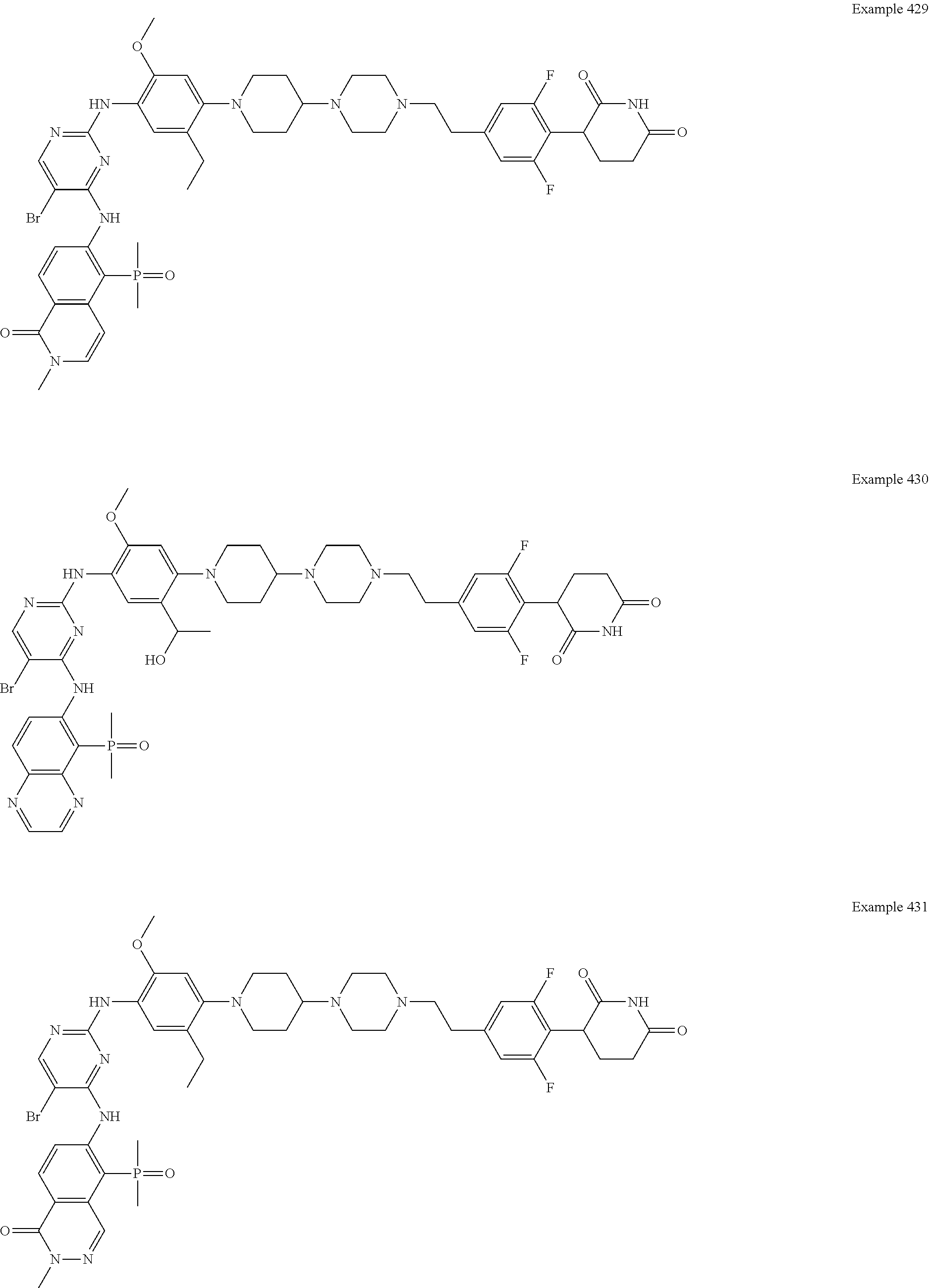 [SMILES: CCc1cc(Nc2ncc(Br)c(Nc3ccc4c(=O)n(C)ccc4c3P(C)(C)=O)n2)c(OC)cc1N1CCC(N2CCN(CCc3cc(F)c(C4CCC(=O)NC4=O)c(F)c3)CC2)CC1 ; COc1cc(N2CCC(N3CCN(CCc4cc(F)c(C5CCC(=O)NC5=O)c(F)c4)CC3)CC2)c(C(C)O)cc1Nc1ncc(Br)c(Nc2ccc3nccnc3c2P(C)(C)=O)n1 ; CCc1cc(Nc2ncc(Br)c(Nc3ccc4c(=O)n(C)ncc4c3P(C)(C)=O)n2)c(OC)cc1N1CCC(N2CCN(CCc3cc(F)c(C4CCC(=O)NC4=O)c(F)c3)CC2)CC1]

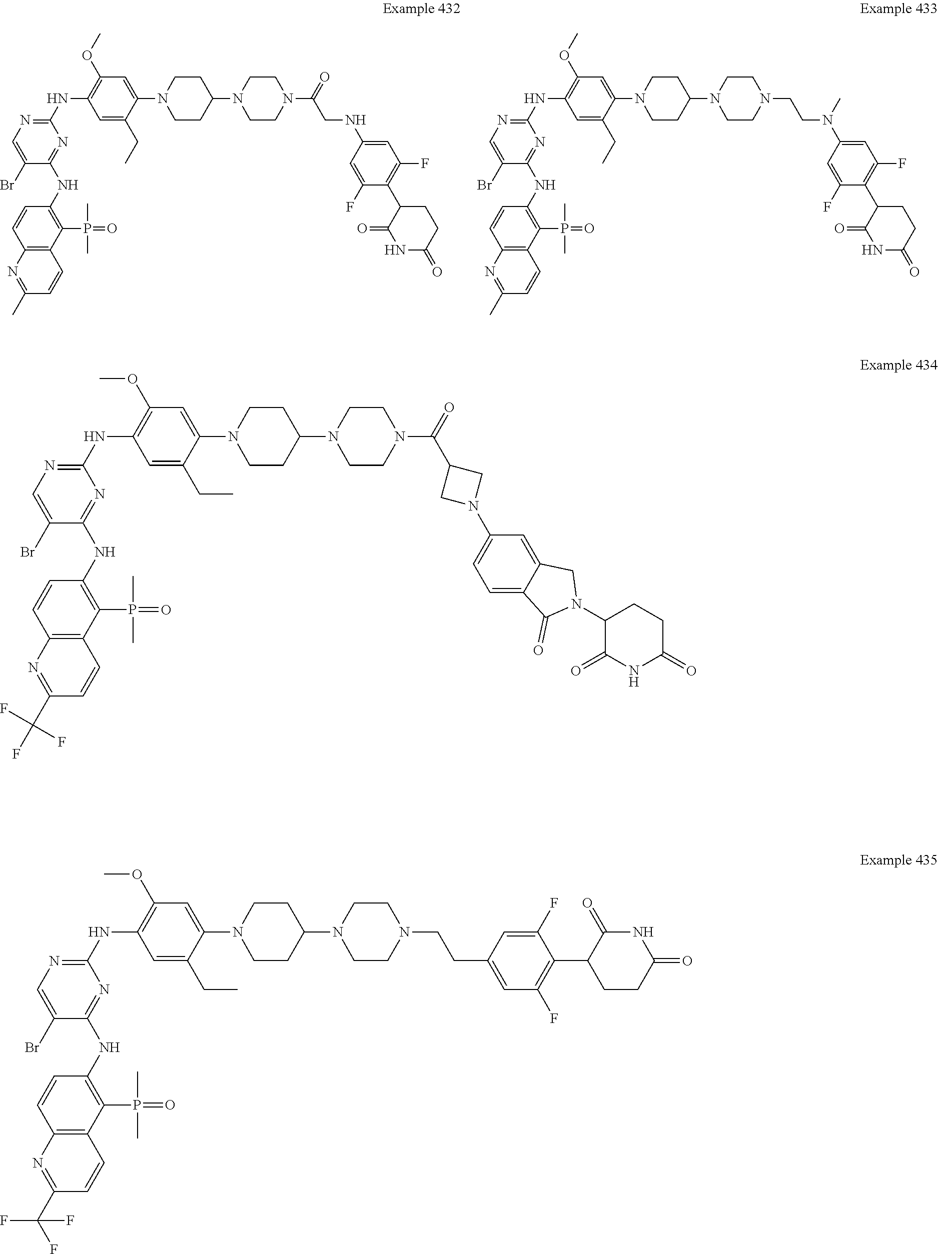 [SMILES: CCc1cc(Nc2ncc(Br)c(Nc3ccc4nc(C)ccc4c3P(C)(C)=O)n2)c(OC)cc1N1CCC(N2CCN(C(=O)CNc3cc(F)c(C4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CCN(CCN(C)c3cc(F)c(C4CCC(=O)NC4=O)c(F)c3)CC2)CC1 ; CCc1cc(Nc2ncc(Br)c(Nc3ccc4nc(C(F)(F)F)ccc4c3P(C)(C)=O)n2)c(OC)cc1N1CCC(N2CCN(C(=O)C3CN(c4ccc5c(c4)CN(C4CCC(=O)NC4=O)C5=O)C3)CC2)CC1 ; CCc1cc(Nc2ncc(Br)c(Nc3ccc4nc(C(F)(F)F)ccc4c3P(C)(C)=O)n2)c(OC)cc1N1CCC(N2CCN(CCc3cc(F)c(C4CCC(=O)NC4=O)c(F)c3)CC2)CC1]

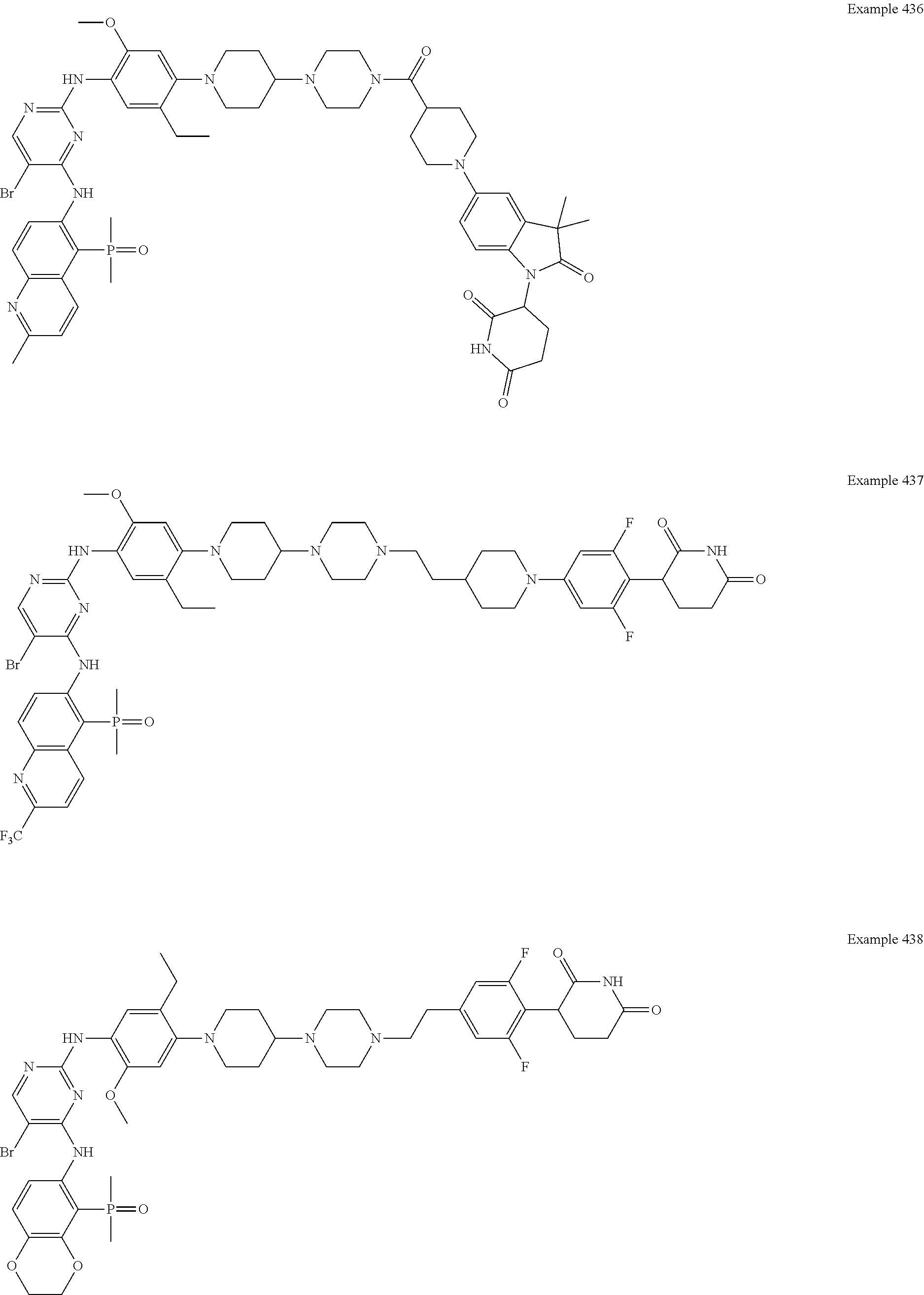 [SMILES: CCc1cc(Nc2ncc(Br)c(Nc3ccc4nc(C)ccc4c3P(C)(C)=O)n2)c(OC)cc1N1CCC(N2CCN(C(=O)C3CCN(c4ccc5c(c4)C(C)(C)C(=O)N5C4CCC(=O)NC4=O)CC3)CC2)CC1 ; CCc1cc(Nc2ncc(Br)c(Nc3ccc4nc(C(F)(F)F)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4c(c3P(C)(C)=O)OCCO4)n2)c(OC)cc1N1CCC(N2CCN(CCc3cc(F)c(C4CCC(=O)NC4=O)c(F)c3)CC2)CC1]

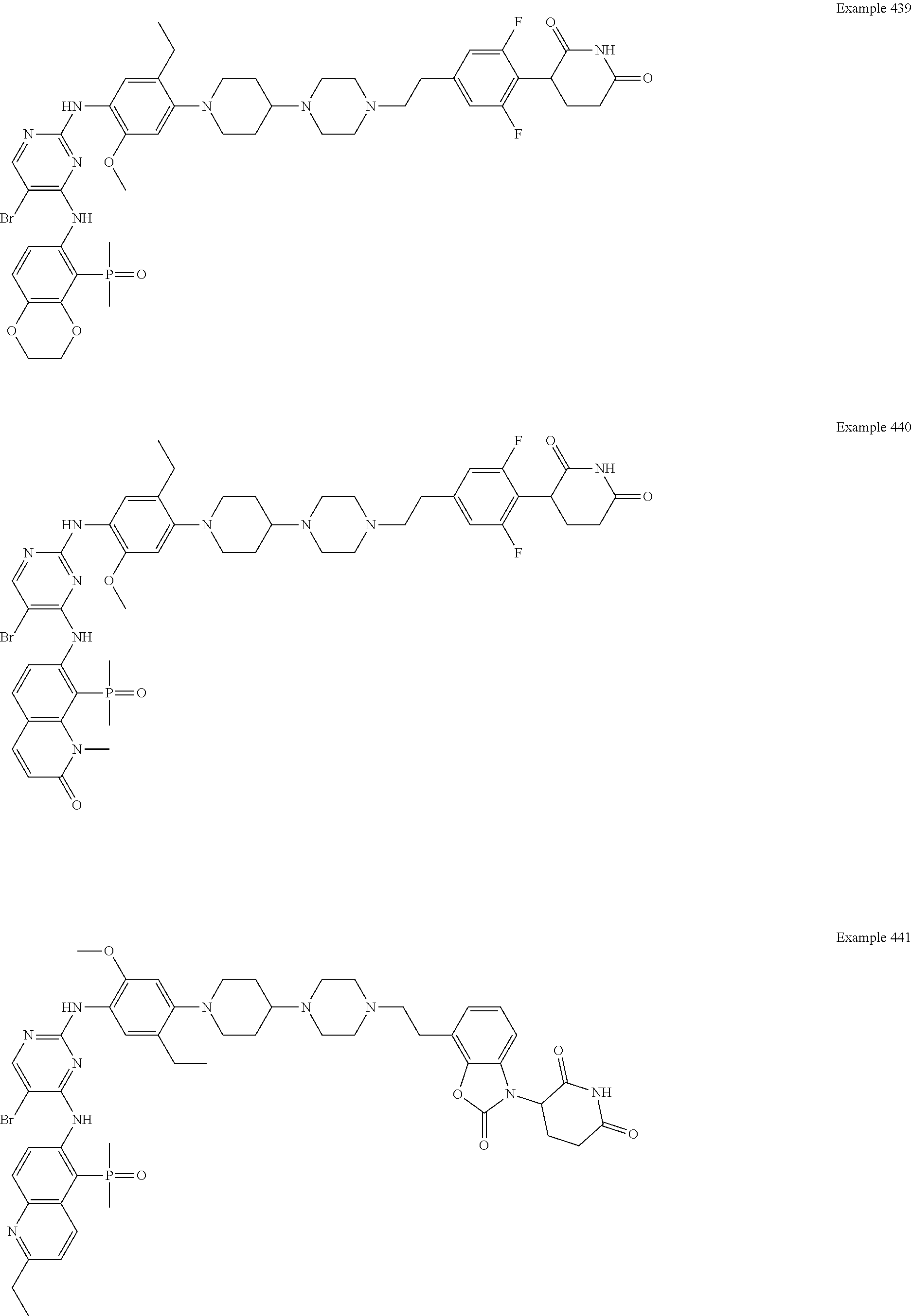 [SMILES: CCc1cc(Nc2ncc(Br)c(Nc3ccc4c(c3P(C)(C)=O)OCCO4)n2)c(OC)cc1N1CCC(N2CCN(CCc3cc(F)c(C4CCC(=O)NC4=O)c(F)c3)CC2)CC1 ; CCc1cc(Nc2ncc(Br)c(Nc3ccc4ccc(=O)n(C)c4c3P(C)(C)=O)n2)c(OC)cc1N1CCC(N2CCN(CCc3cc(F)c(C4CCC(=O)NC4=O)c(F)c3)CC2)CC1 ; CCc1ccc2c(P(C)(C)=O)c(Nc3nc(Nc4cc(CC)c(N5CCC(N6CCN(CCc7cccc8c7oc(=O)n8C7CCC(=O)NC7=O)CC6)CC5)cc4OC)ncc3Br)ccc2n1]

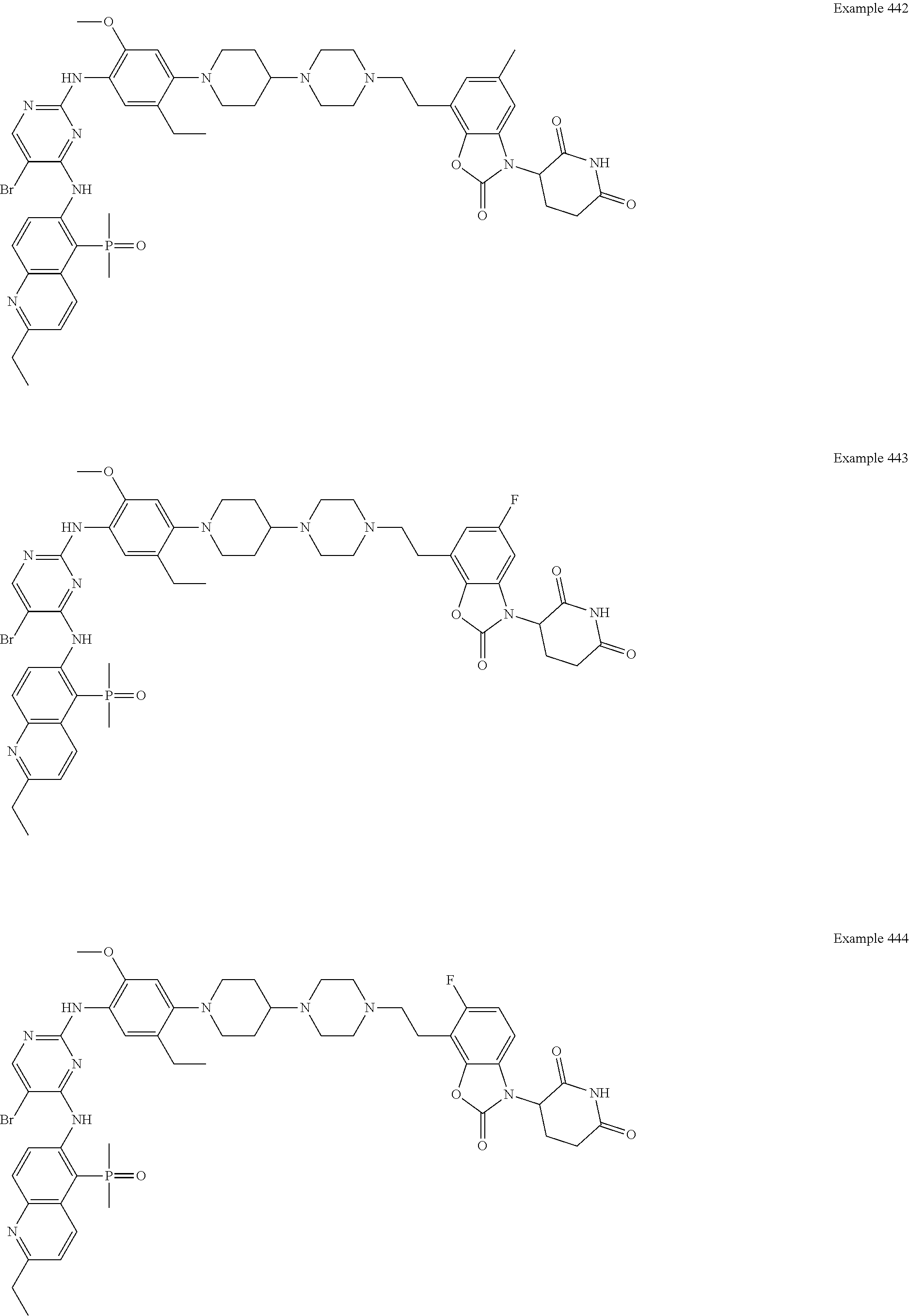 [SMILES: CCc1ccc2c(P(C)(C)=O)c(Nc3nc(Nc4cc(CC)c(N5CCC(N6CCN(CCc7cc(C)cc8c7oc(=O)n8C7CCC(=O)NC7=O)CC6)CC5)cc4OC)ncc3Br)ccc2n1 ; CCc1ccc2c(P(C)(C)=O)c(Nc3nc(Nc4cc(CC)c(N5CCC(N6CCN(CCc7cc(F)cc8c7oc(=O)n8C7CCC(=O)NC7=O)CC6)CC5)cc4OC)ncc3Br)ccc2n1 ; CCc1ccc2c(P(C)(C)=O)c(Nc3nc(Nc4cc(CC)c(N5CCC(N6CCN(CCc7c(F)ccc8c7oc(=O)n8C7CCC(=O)NC7=O)CC6)CC5)cc4OC)ncc3Br)ccc2n1]

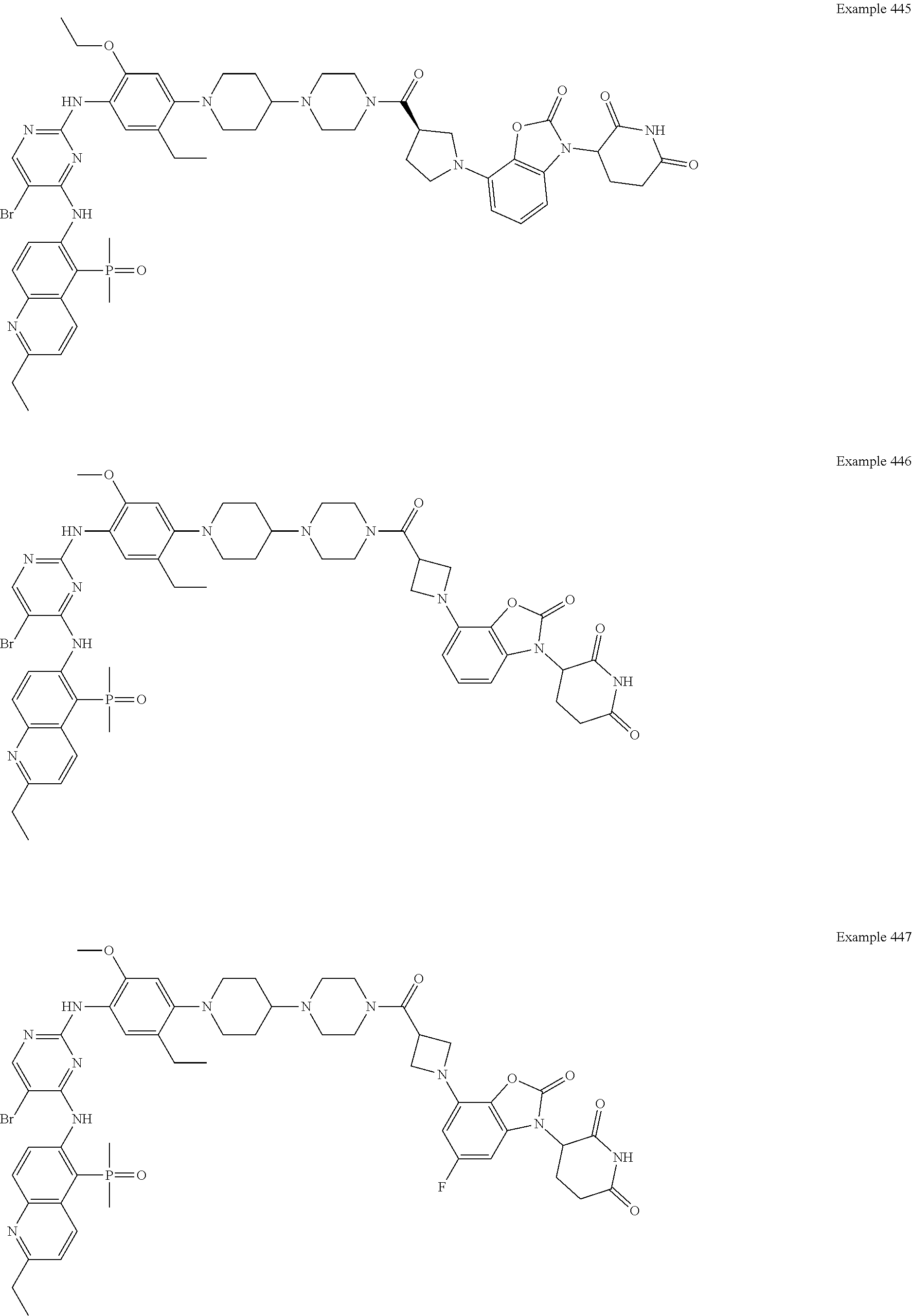 [SMILES: CCOc1cc(N2CCC(N3CCN(C(=O)[C@@H]4CCN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1 ; CCc1ccc2c(P(C)(C)=O)c(Nc3nc(Nc4cc(CC)c(N5CCC(N6CCN(C(=O)C7CN(c8cccc9c8oc(=O)n9C8CCC(=O)NC8=O)C7)CC6)CC5)cc4OC)ncc3Br)ccc2n1 ; CCc1ccc2c(P(C)(C)=O)c(Nc3nc(Nc4cc(CC)c(N5CCC(N6CCN(C(=O)C7CN(c8cc(F)cc9c8oc(=O)n9C8CCC(=O)NC8=O)C7)CC6)CC5)cc4OC)ncc3Br)ccc2n1]

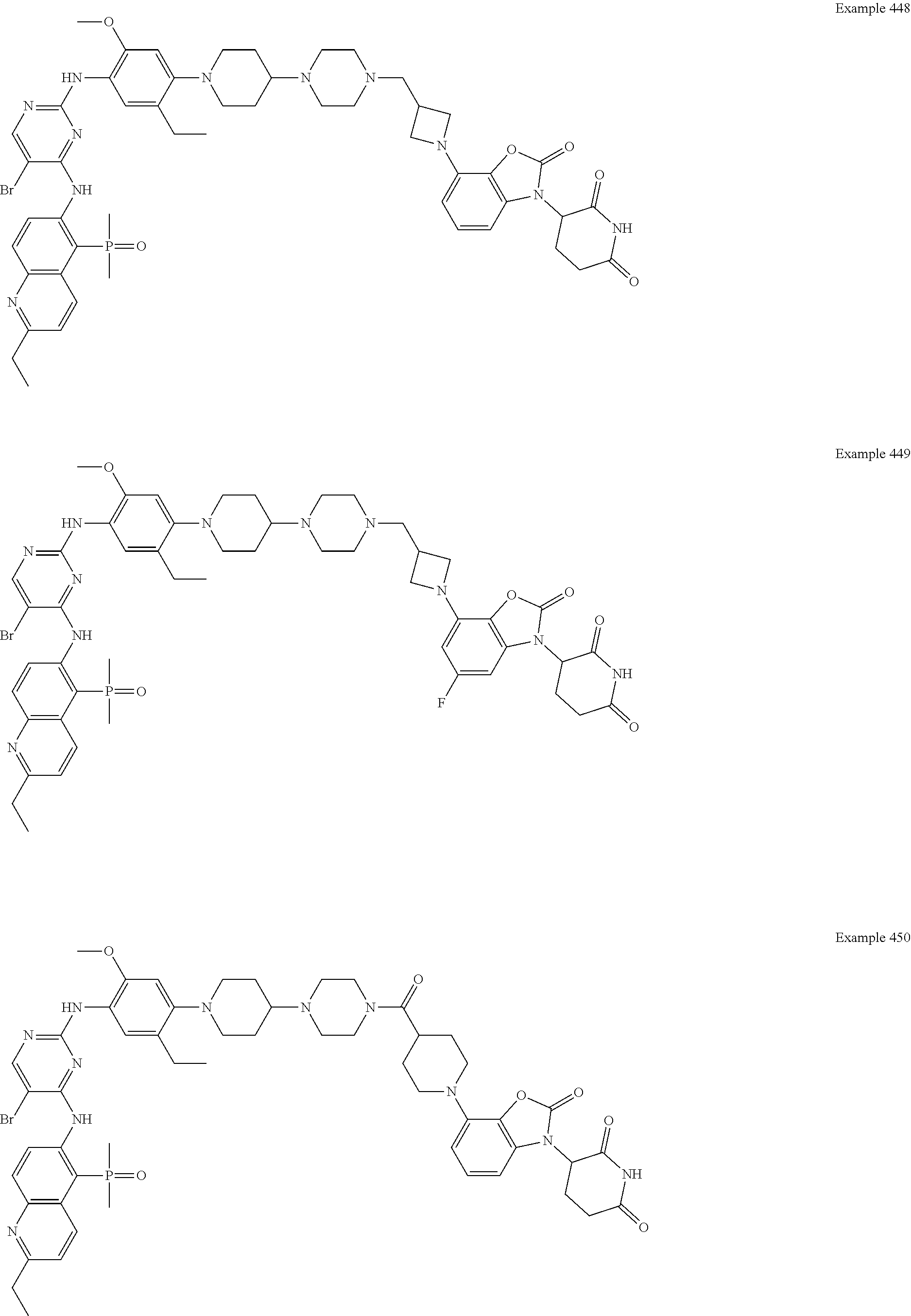 [SMILES: CCc1ccc2c(P(C)(C)=O)c(Nc3nc(Nc4cc(CC)c(N5CCC(N6CCN(CC7CN(c8cccc9c8oc(=O)n9C8CCC(=O)NC8=O)C7)CC6)CC5)cc4OC)ncc3Br)ccc2n1 ; CCc1ccc2c(P(C)(C)=O)c(Nc3nc(Nc4cc(CC)c(N5CCC(N6CCN(CC7CN(c8cc(F)cc9c8oc(=O)n9C8CCC(=O)NC8=O)C7)CC6)CC5)cc4OC)ncc3Br)ccc2n1 ; CCc1ccc2c(P(C)(C)=O)c(Nc3nc(Nc4cc(CC)c(N5CCC(N6CCN(C(=O)C7CCN(c8cccc9c8oc(=O)n9C8CCC(=O)NC8=O)CC7)CC6)CC5)cc4OC)ncc3Br)ccc2n1]

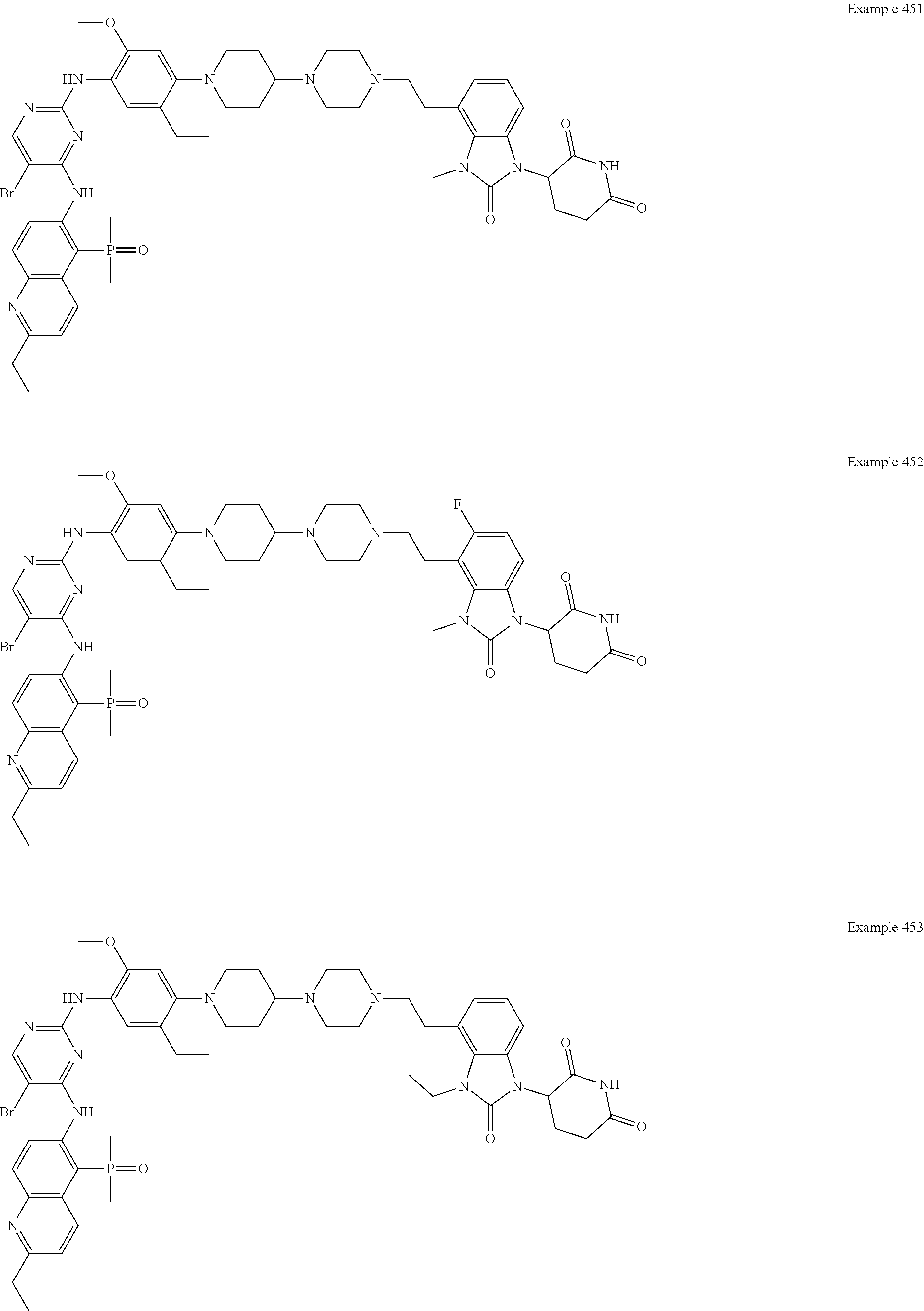 [SMILES: CCc1ccc2c(P(C)(C)=O)c(Nc3nc(Nc4cc(CC)c(N5CCC(N6CCN(CCc7cccc8c7n(C)c(=O)n8C7CCC(=O)NC7=O)CC6)CC5)cc4OC)ncc3Br)ccc2n1 ; CCc1ccc2c(P(C)(C)=O)c(Nc3nc(Nc4cc(CC)c(N5CCC(N6CCN(CCc7c(F)ccc8c7n(C)c(=O)n8C7CCC(=O)NC7=O)CC6)CC5)cc4OC)ncc3Br)ccc2n1 ; CCc1ccc2c(P(C)(C)=O)c(Nc3nc(Nc4cc(CC)c(N5CCC(N6CCN(CCc7cccc8c7n(CC)c(=O)n8C7CCC(=O)NC7=O)CC6)CC5)cc4OC)ncc3Br)ccc2n1]

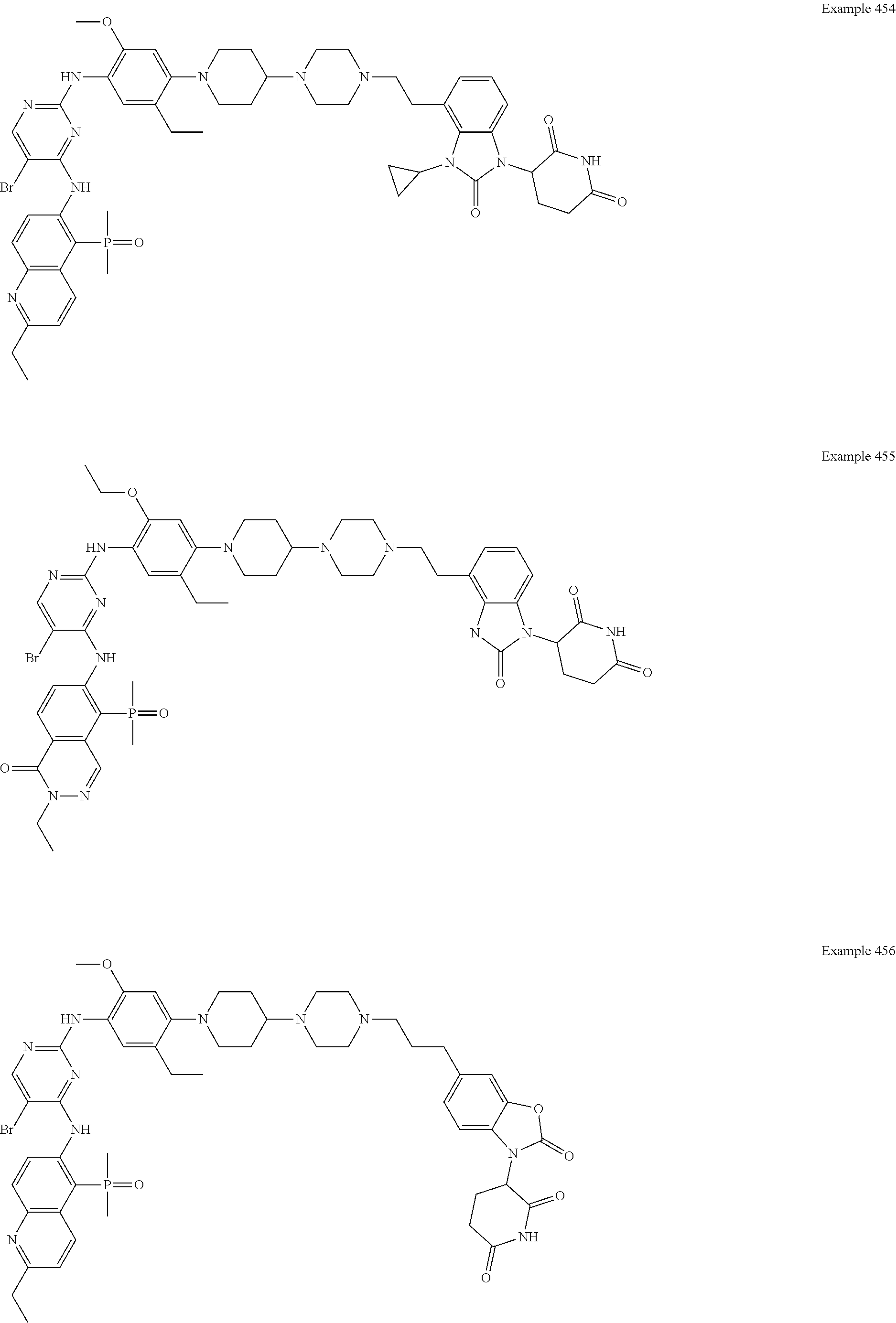 [SMILES: CCc1ccc2c(P(C)(C)=O)c(Nc3nc(Nc4cc(CC)c(N5CCC(N6CCN(CCc7cccc8c7n(C7CC7)c(=O)n8C7CCC(=O)NC7=O)CC6)CC5)cc4OC)ncc3Br)ccc2n1 ; CCOc1cc(N2CCC(N3CCN(CCc4cccc5c4[nH]c(=O)n5C4CCC(=O)NC4=O)CC3)CC2)c(CC)cc1Nc1ncc(Br)c(Nc2ccc3c(=O)n(CC)ncc3c2P(C)(C)=O)n1 ; CCc1ccc2c(P(C)(C)=O)c(Nc3nc(Nc4cc(CC)c(N5CCC(N6CCN(CCCc7ccc8c(c7)oc(=O)n8C7CCC(=O)NC7=O)CC6)CC5)cc4OC)ncc3Br)ccc2n1]

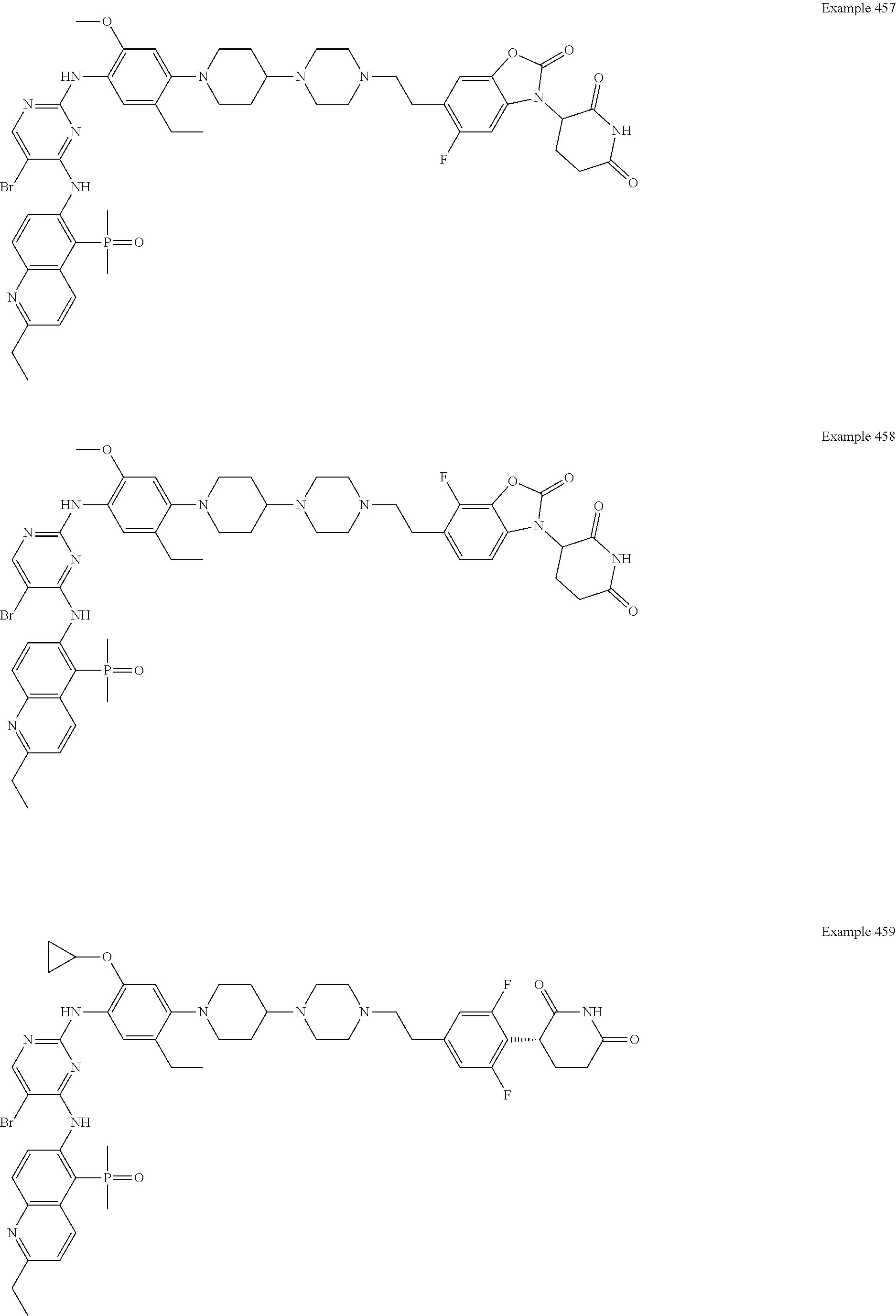 [SMILES: CCc1ccc2c(P(C)(C)=O)c(Nc3nc(Nc4cc(CC)c(N5CCC(N6CCN(CCc7cc8oc(=O)n(C9CCC(=O)NC9=O)c8cc7F)CC6)CC5)cc4OC)ncc3Br)ccc2n1 ; CCc1ccc2c(P(C)(C)=O)c(Nc3nc(Nc4cc(CC)c(N5CCC(N6CCN(CCc7ccc8c(oc(=O)n8C8CCC(=O)NC8=O)c7F)CC6)CC5)cc4OC)ncc3Br)ccc2n1 ; CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC4CC4)ncc3Br)ccc2n1]

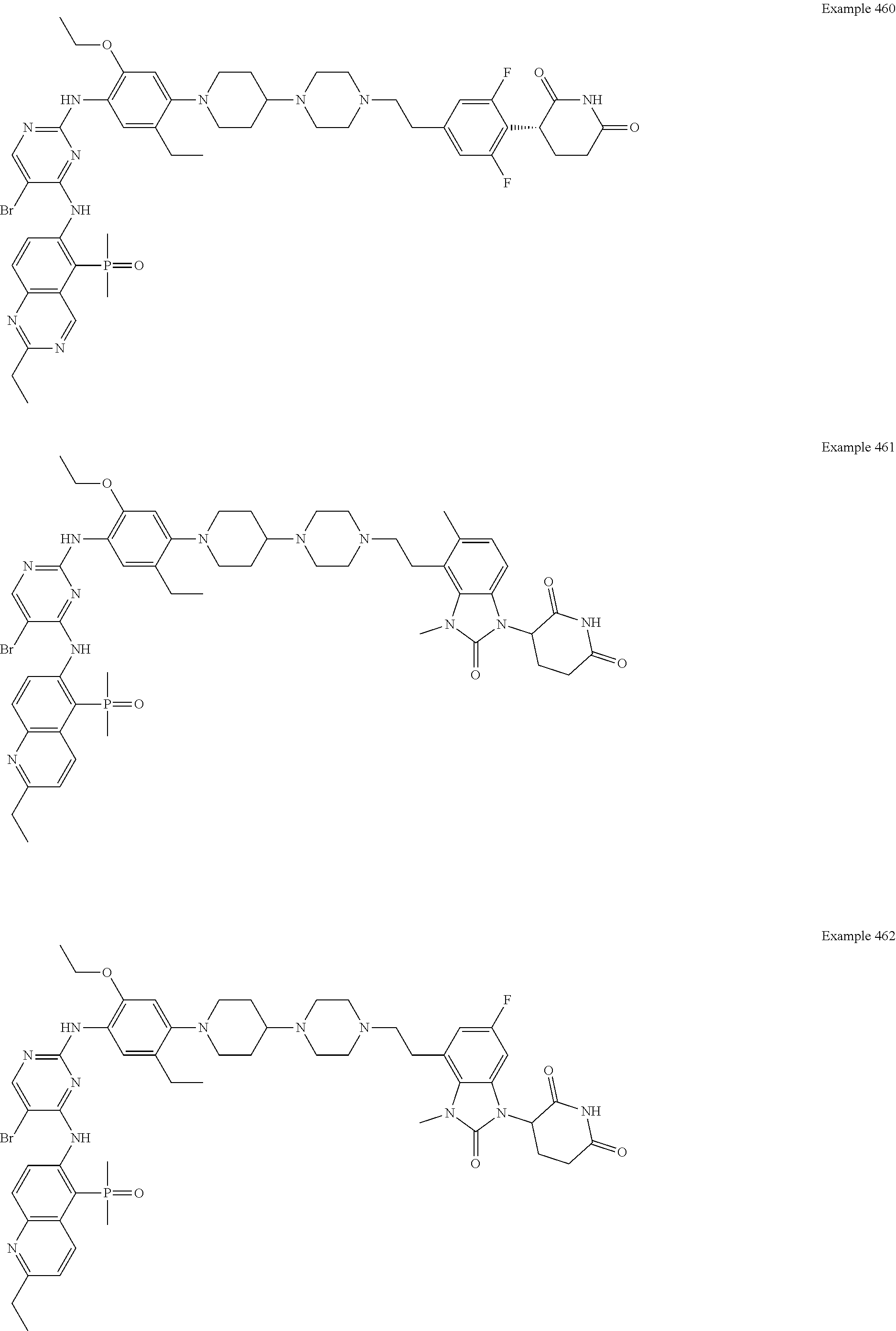 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)ncc3c2P(C)(C)=O)n1 ; CCOc1cc(N2CCC(N3CCN(CCc4c(C)ccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cc(F)cc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

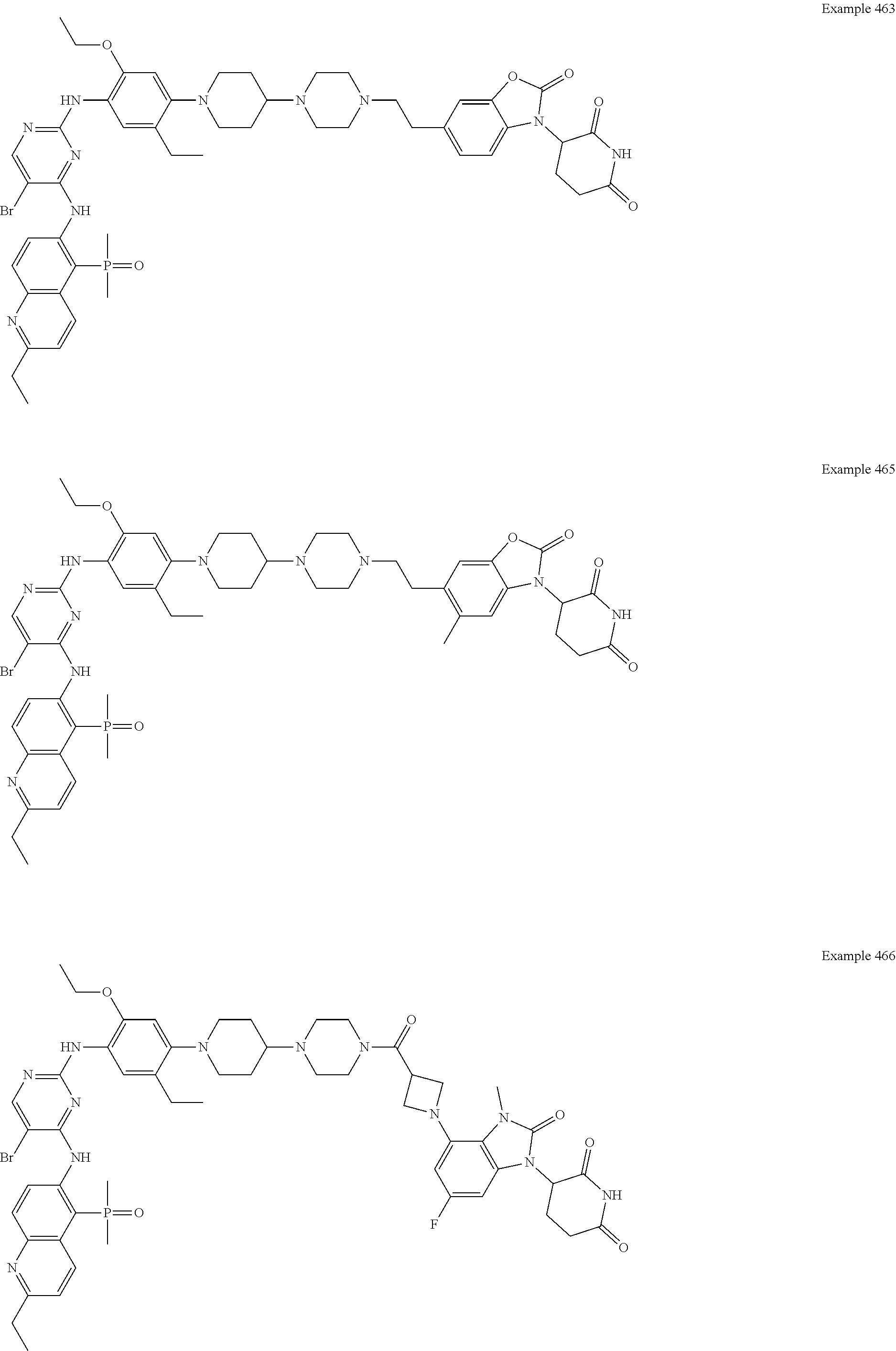 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cc5oc(=O)n(C6CCC(=O)NC6=O)c5cc4C)CC3)CC2)c(CC)cc1Nc1ncc(Br)c(Nc2ccc3nc(CC)ccc3c2P(C)(C)=O)n1 ; CCOc1cc(N2CCC(N3CCN(C(=O)C4CN(c5cc(F)cc6c5n(C)c(=O)n6C5CCC(=O)NC5=O)C4)CC3)CC2)c(CC)cc1Nc1ncc(Br)c(Nc2ccc3nc(CC)ccc3c2P(C)(C)=O)n1]

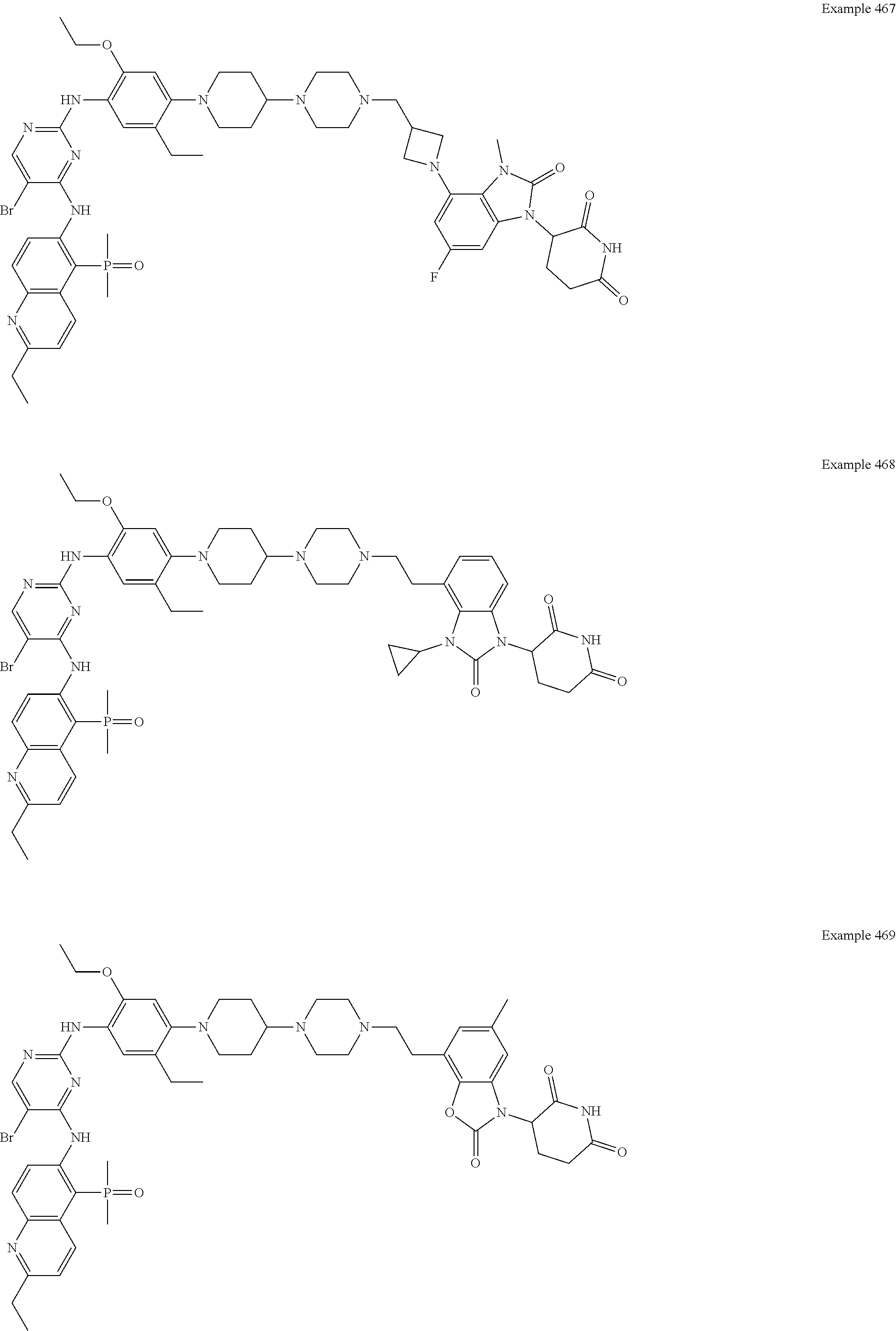 [SMILES: CCOc1cc(N2CCC(N3CCN(CC4CN(c5cc(F)cc6c5n(C)c(=O)n6C5CCC(=O)NC5=O)C4)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cccc5c4n(C4CC4)c(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cc(C)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

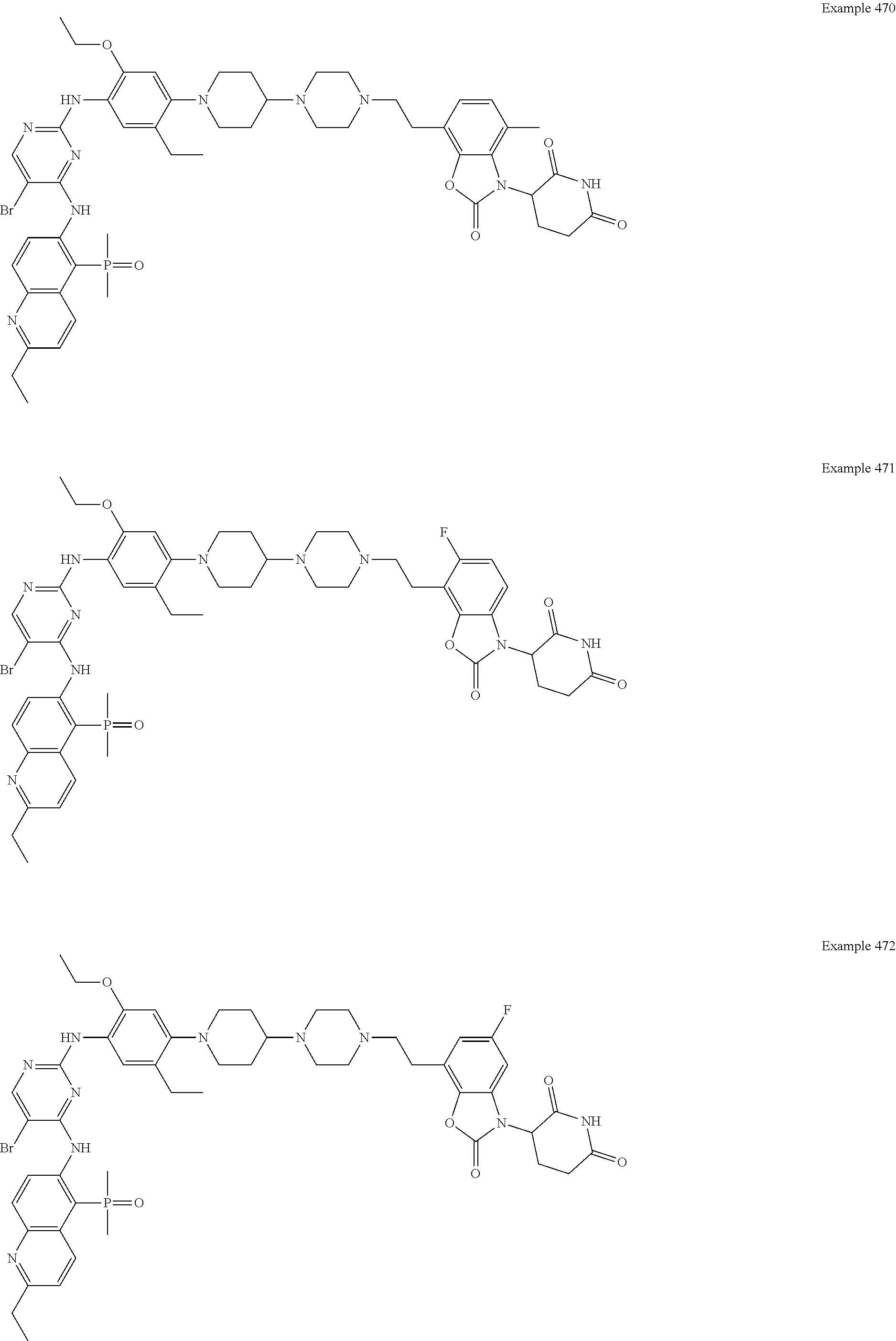 [SMILES: CCOc1cc(N2CCC(N3CCN(CCc4ccc(C)c5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCc4c(F)ccc5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cc(F)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

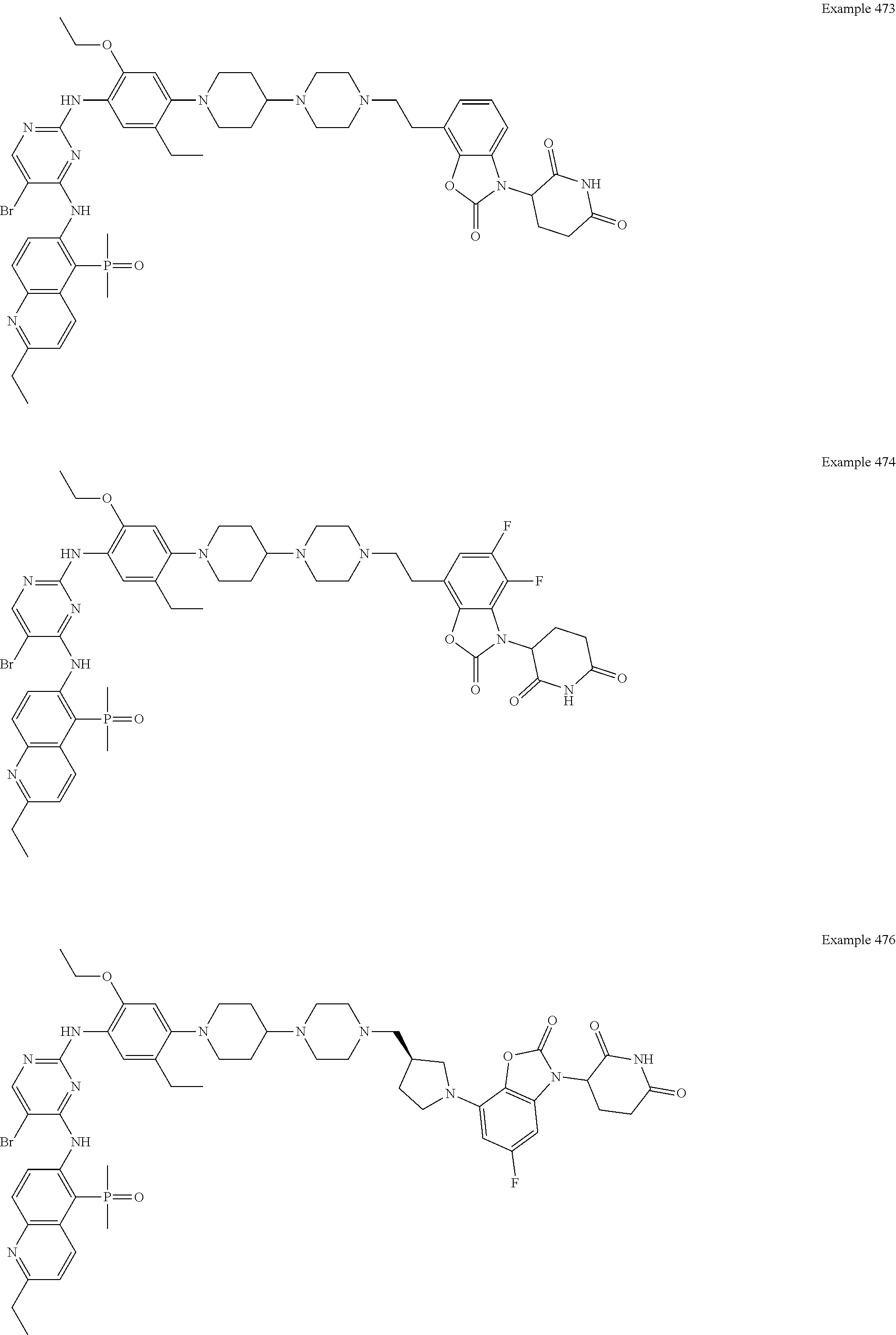 [SMILES: CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cc(F)c(F)c5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(C[C@@H]4CCN(c5cc(F)cc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

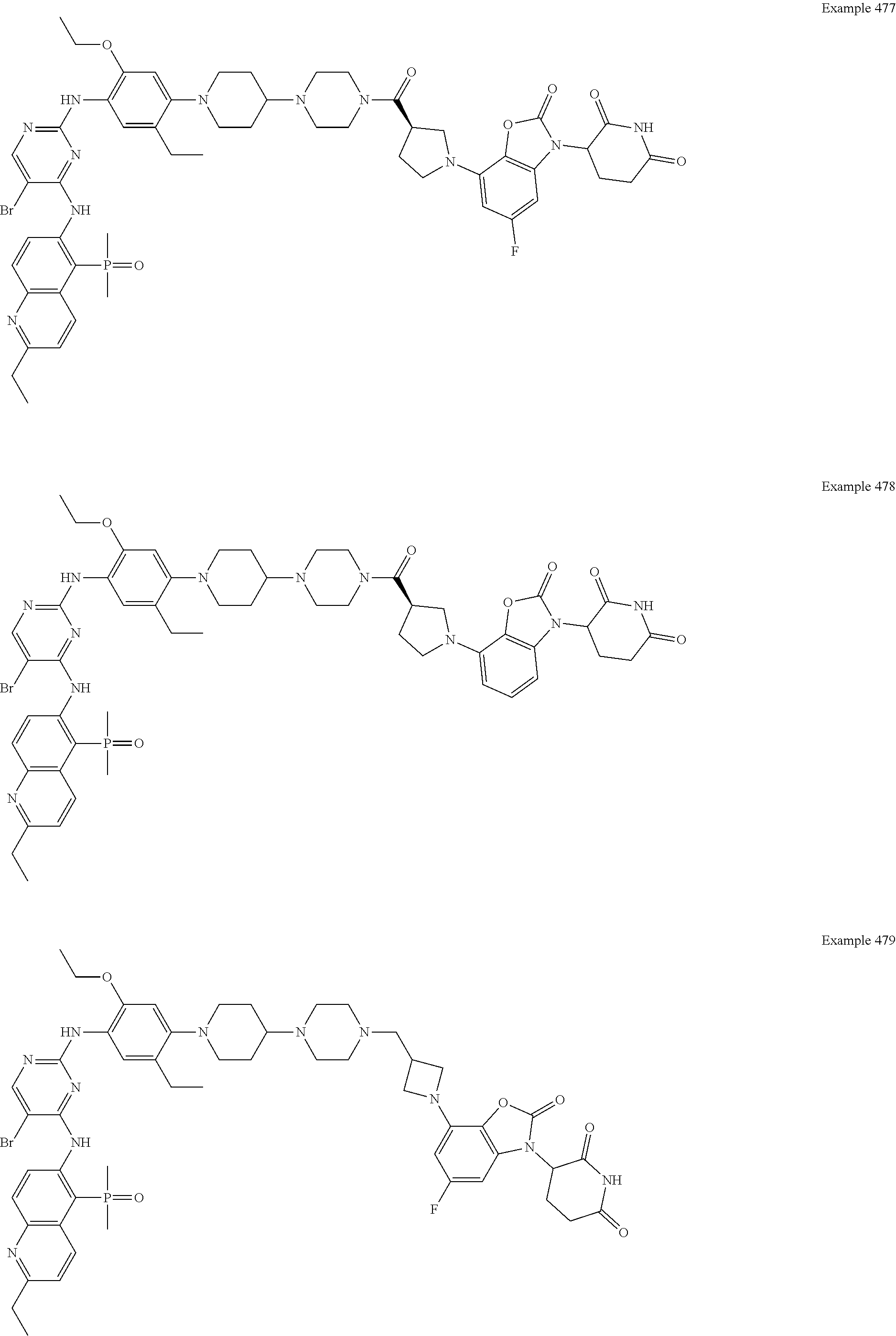 [SMILES: CCOc1cc(N2CCC(N3CCN(C(=O)[C@@H]4CCN(c5cc(F)cc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1 ; CCOc1cc(N2CCC(N3CCN(C(=O)[C@@H]4CCN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1 ; CCOc1cc(N2CCC(N3CCN(CC4CN(c5cc(F)cc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

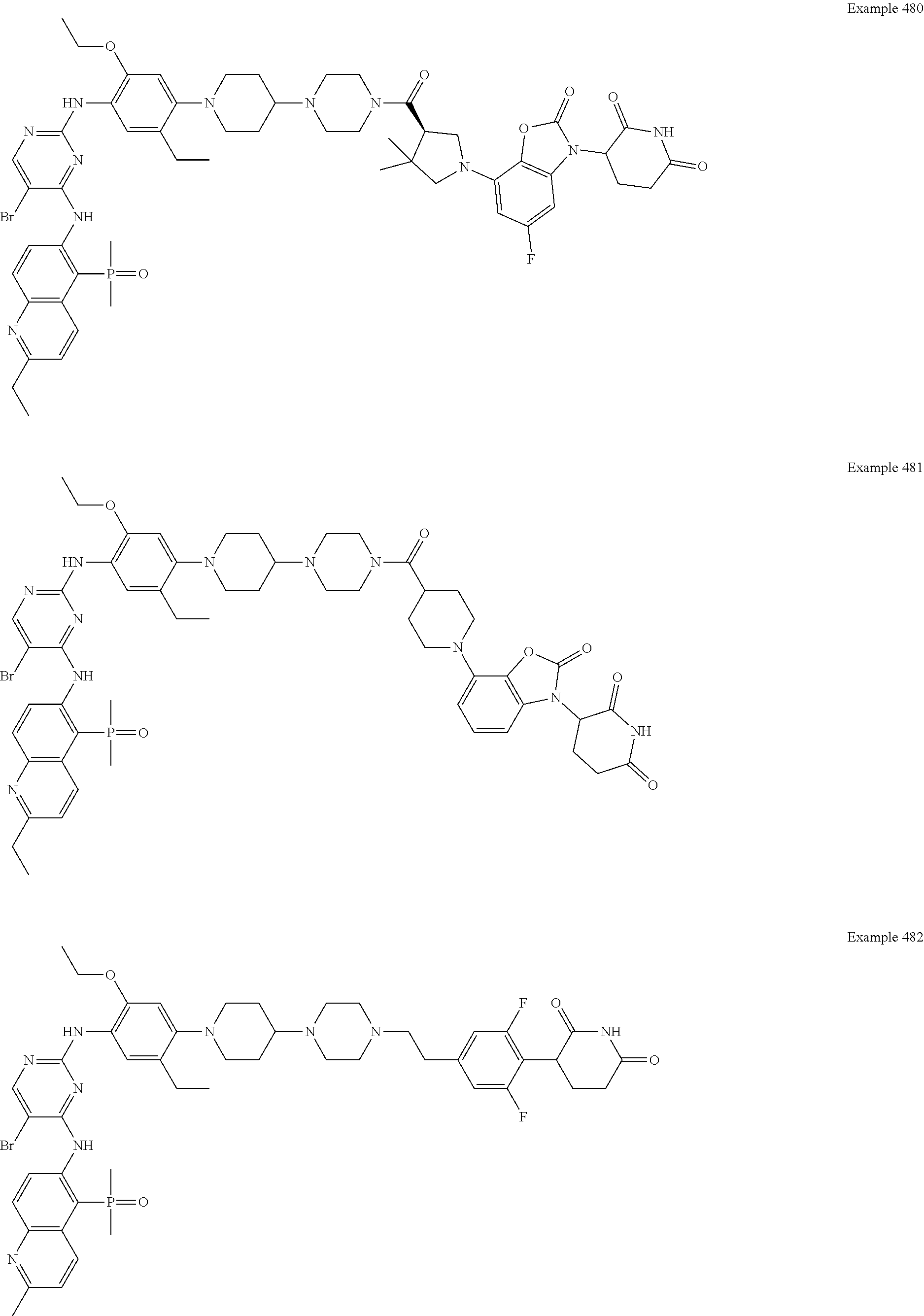 [SMILES: CCOc1cc(N2CCC(N3CCN(C(=O)[C@H]4CN(c5cc(F)cc6c5oc(=O)n6C5CCC(=O)NC5=O)CC4(C)C)CC3)CC2)c(CC)cc1Nc1ncc(Br)c(Nc2ccc3nc(CC)ccc3c2P(C)(C)=O)n1 ; CCOc1cc(N2CCC(N3CCN(C(=O)C4CCN(c5cccc6c5oc(=O)n6C5CCC(=O)NC5=O)CC4)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cc(F)c(C5CCC(=O)NC5=O)c(F)c4)CC3)CC2)c(CC)cc1Nc1ncc(Br)c(Nc2ccc3nc(C)ccc3c2P(C)(C)=O)n1]

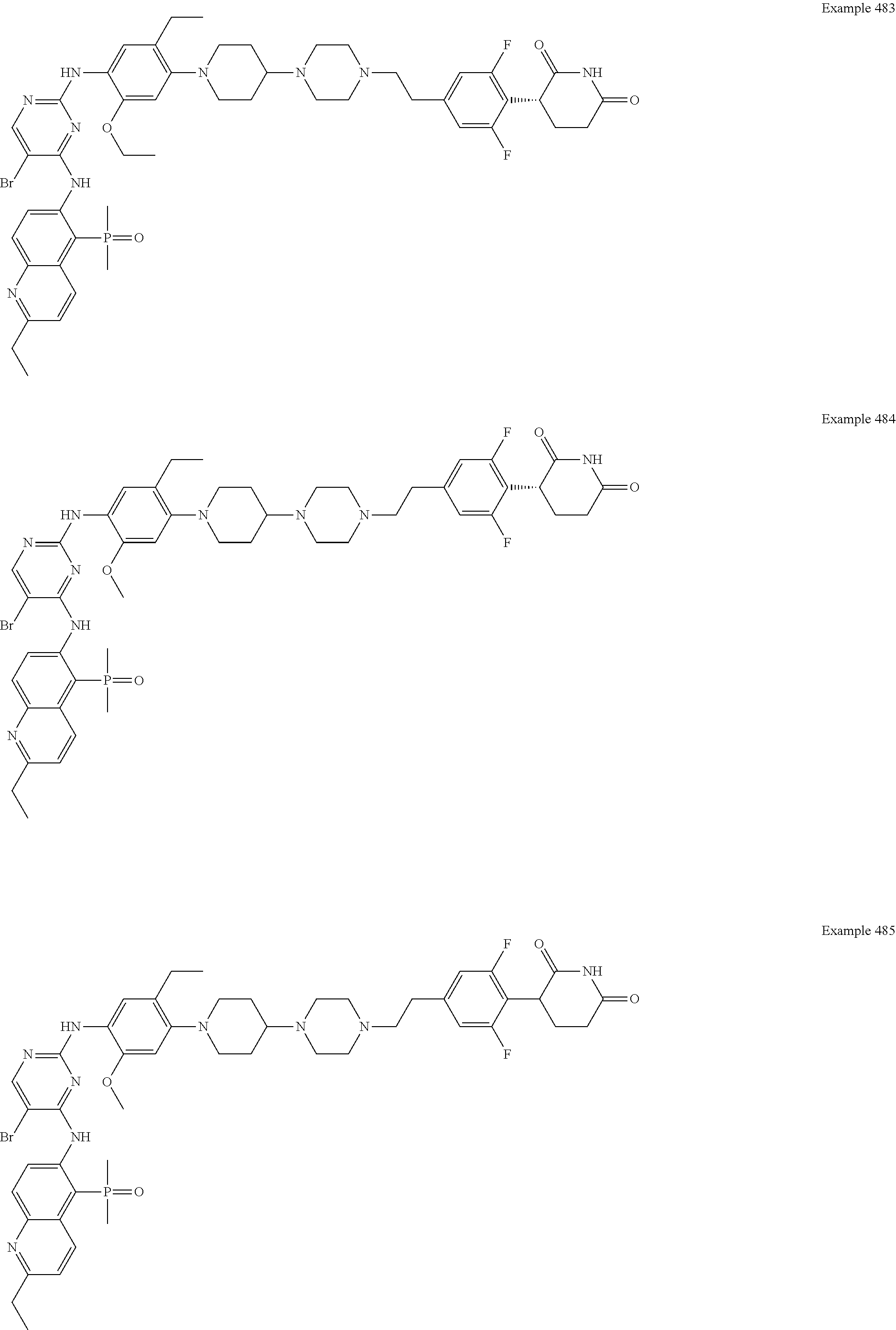 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)ccc3c2P(C)(C)=O)n1 ; CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC)ncc3Br)ccc2n1 ; CCc1ccc2c(P(C)(C)=O)c(Nc3nc(Nc4cc(CC)c(N5CCC(N6CCN(CCc7cc(F)c(C8CCC(=O)NC8=O)c(F)c7)CC6)CC5)cc4OC)ncc3Br)ccc2n1]

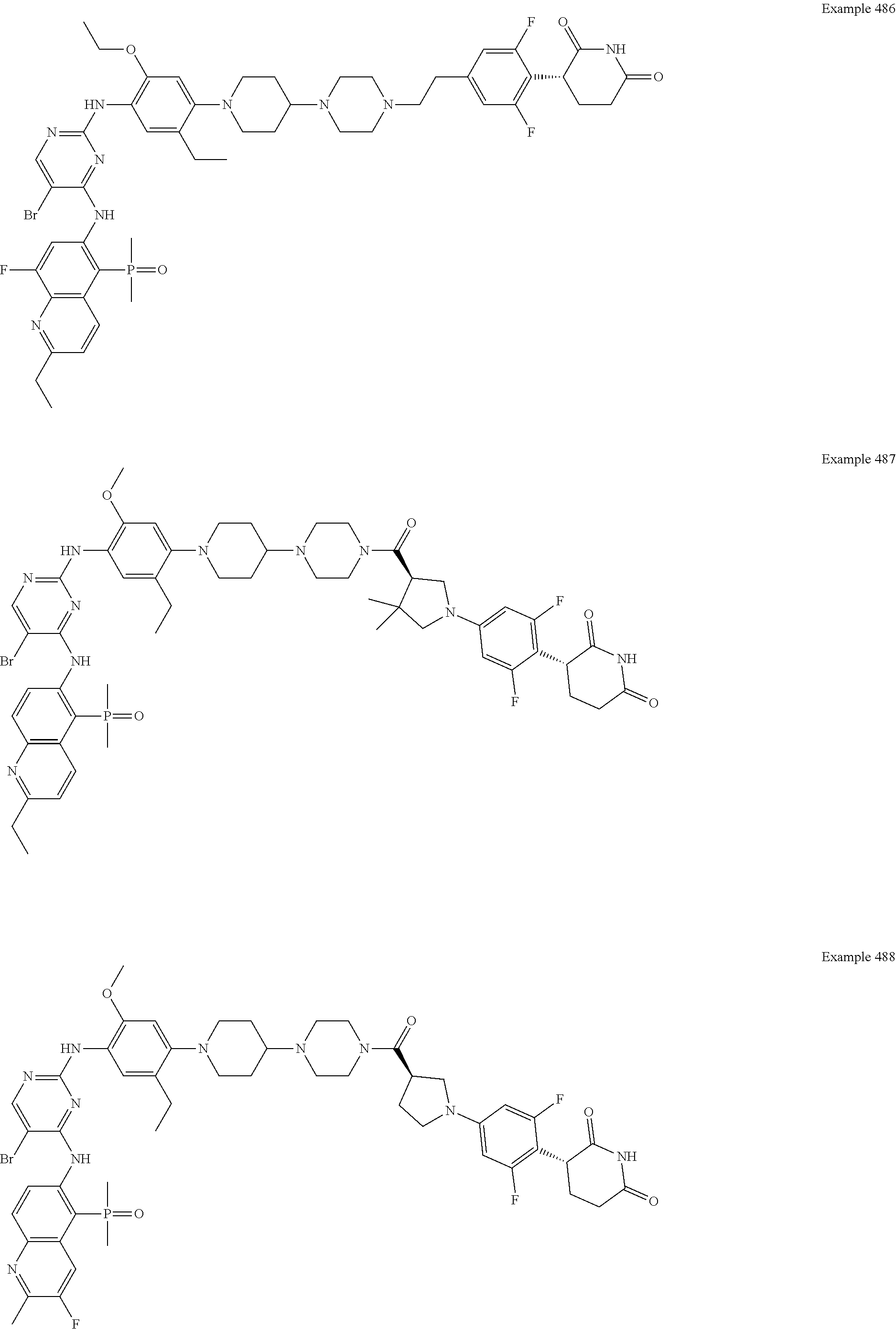 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2cc(F)c3nc(CC)ccc3c2P(C)(C)=O)n1 ; CCc1ccc2c(P(C)(C)=O)c(Nc3nc(Nc4cc(CC)c(N5CCC(N6CCN(C(=O)[C@H]7CN(c8cc(F)c([C@H]9CCC(=O)NC9=O)c(F)c8)CC7(C)C)CC6)CC5)cc4OC)ncc3Br)ccc2n1 ; CCc1cc(Nc2ncc(Br)c(Nc3ccc4nc(C)c(F)cc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

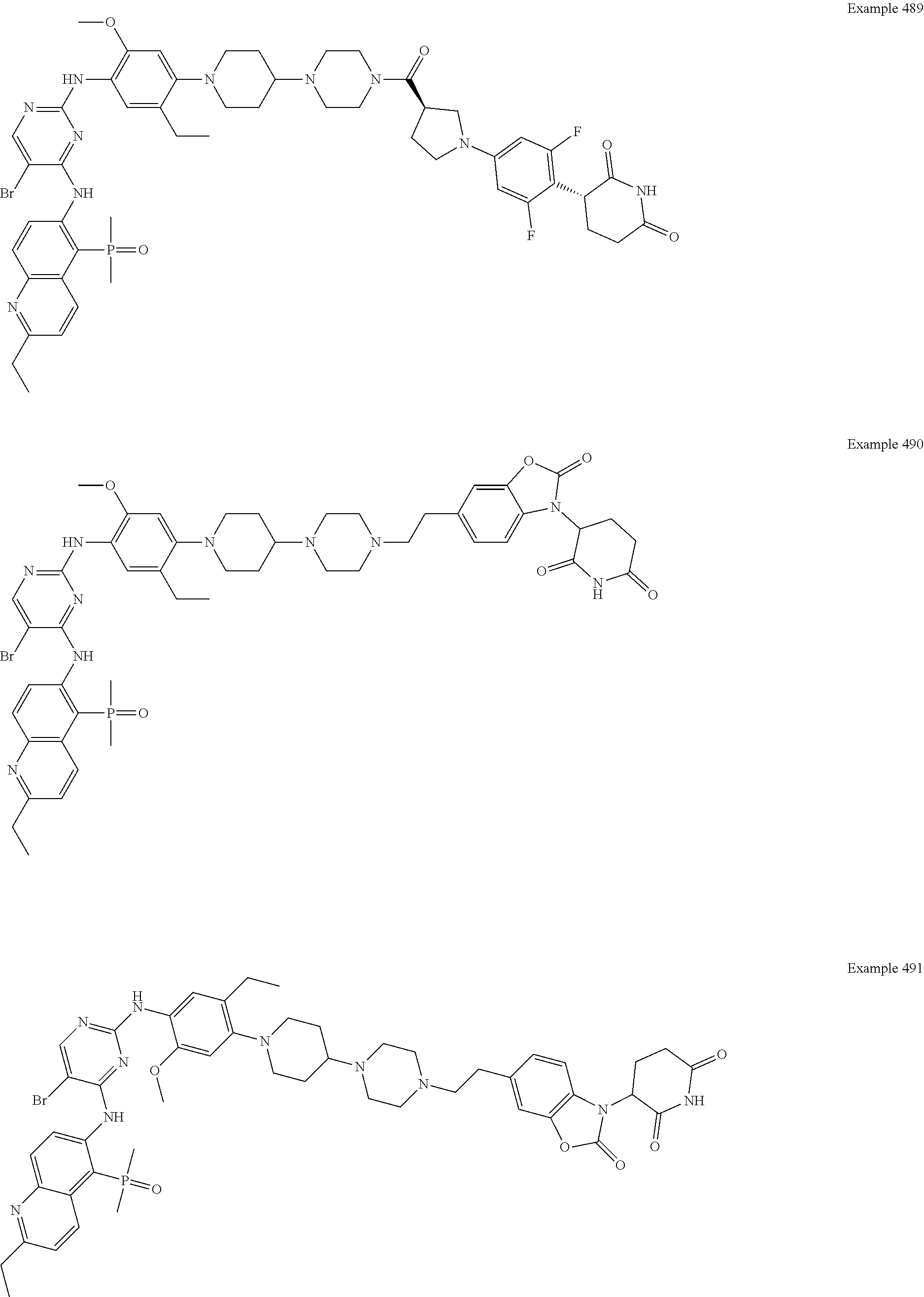 [SMILES: CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1 ; CCc1ccc2c(P(C)(C)=O)c(Nc3nc(Nc4cc(CC)c(N5CCC(N6CCN(CCc7ccc8c(c7)oc(=O)n8C7CCC(=O)NC7=O)CC6)CC5)cc4OC)ncc3Br)ccc2n1 ; CCc1ccc2c(P(C)(C)=O)c(Nc3nc(Nc4cc(CC)c(N5CCC(N6CCN(CCc7ccc8c(c7)oc(=O)n8C7CCC(=O)NC7=O)CC6)CC5)cc4OC)ncc3Br)ccc2n1]

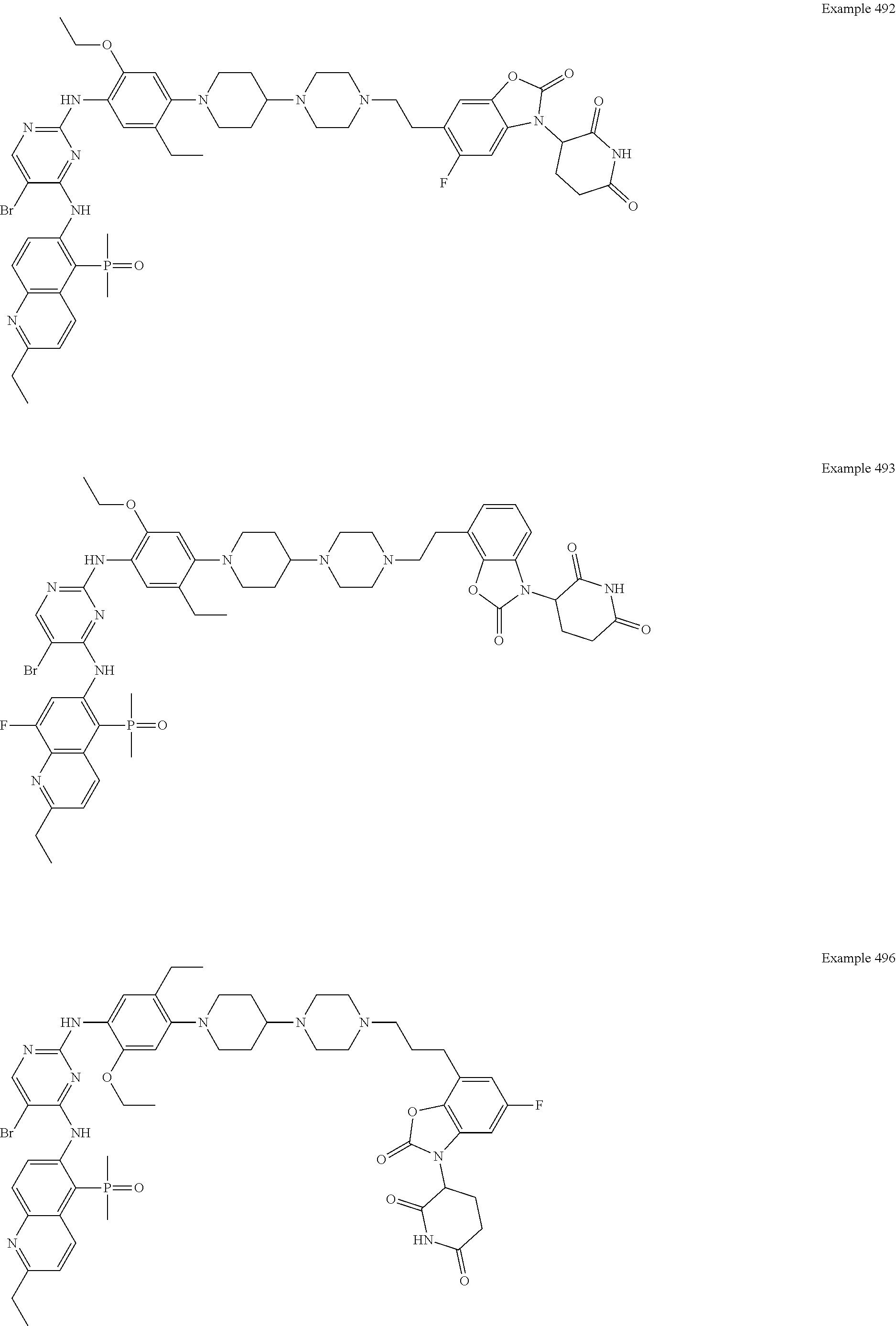 [SMILES: CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cccc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1 ; CCOc1cc(N2CCC(N3CCN(CCCc4cc(F)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

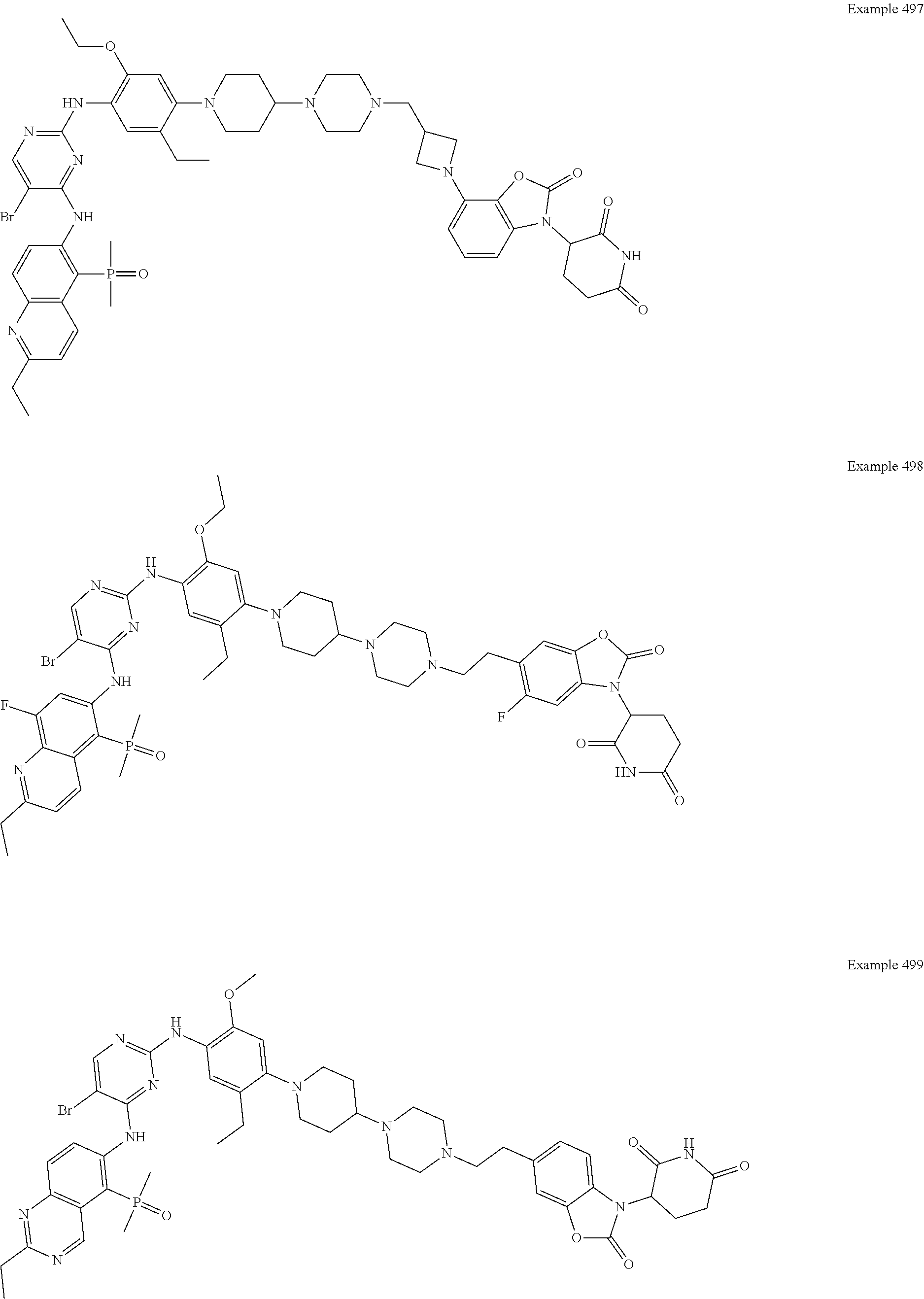 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2cc(F)c3nc(CC)ccc3c2P(C)(C)=O)n1 ; CCc1ncc2c(P(C)(C)=O)c(Nc3nc(Nc4cc(CC)c(N5CCC(N6CCN(CCc7ccc8c(c7)oc(=O)n8C7CCC(=O)NC7=O)CC6)CC5)cc4OC)ncc3Br)ccc2n1]

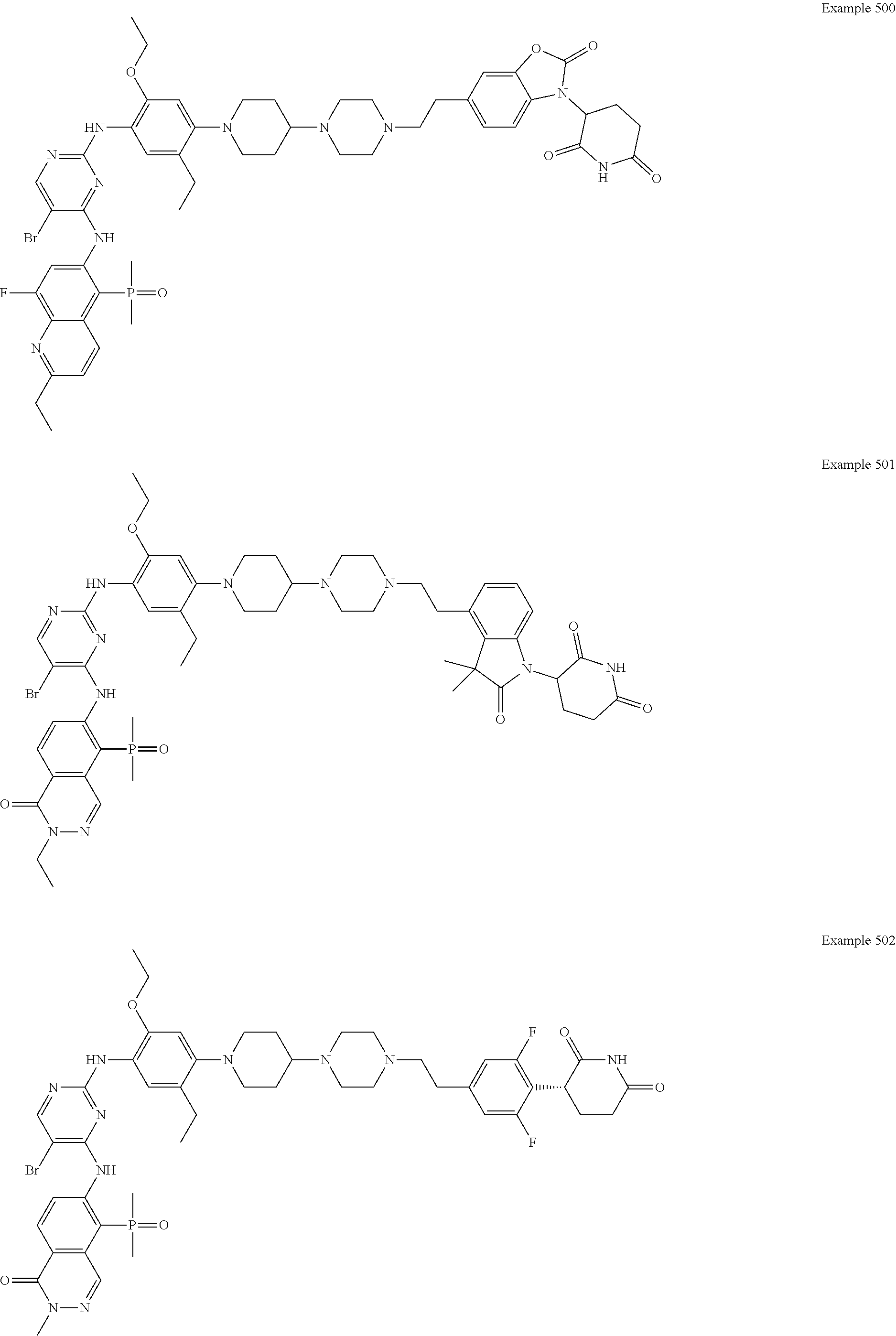 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2cc(F)c3nc(CC)ccc3c2P(C)(C)=O)n1 ; CCOc1cc(N2CCC(N3CCN(CCc4cccc5c4C(C)(C)C(=O)N5C4CCC(=O)NC4=O)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3c(=O)n(C)ncc3c2P(C)(C)=O)n1]

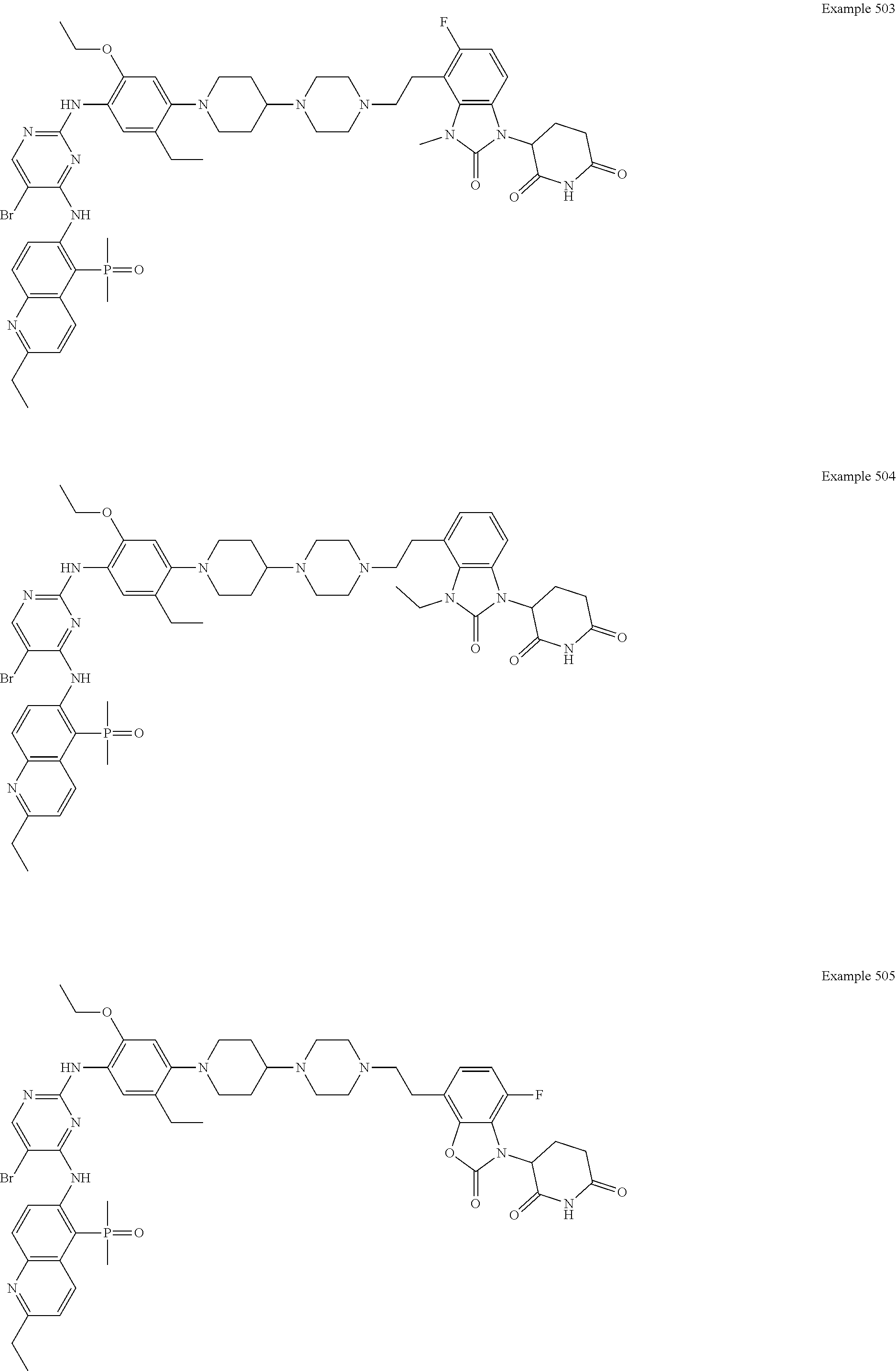 [SMILES: CCOc1cc(N2CCC(N3CCN(CCc4c(F)ccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCc4ccc(F)c5c4oc(=O)n5C4CCC(=O)NC4=O)CC3)CC2)c(CC)cc1Nc1ncc(Br)c(Nc2ccc3nc(CC)ccc3c2P(C)(C)=O)n1]

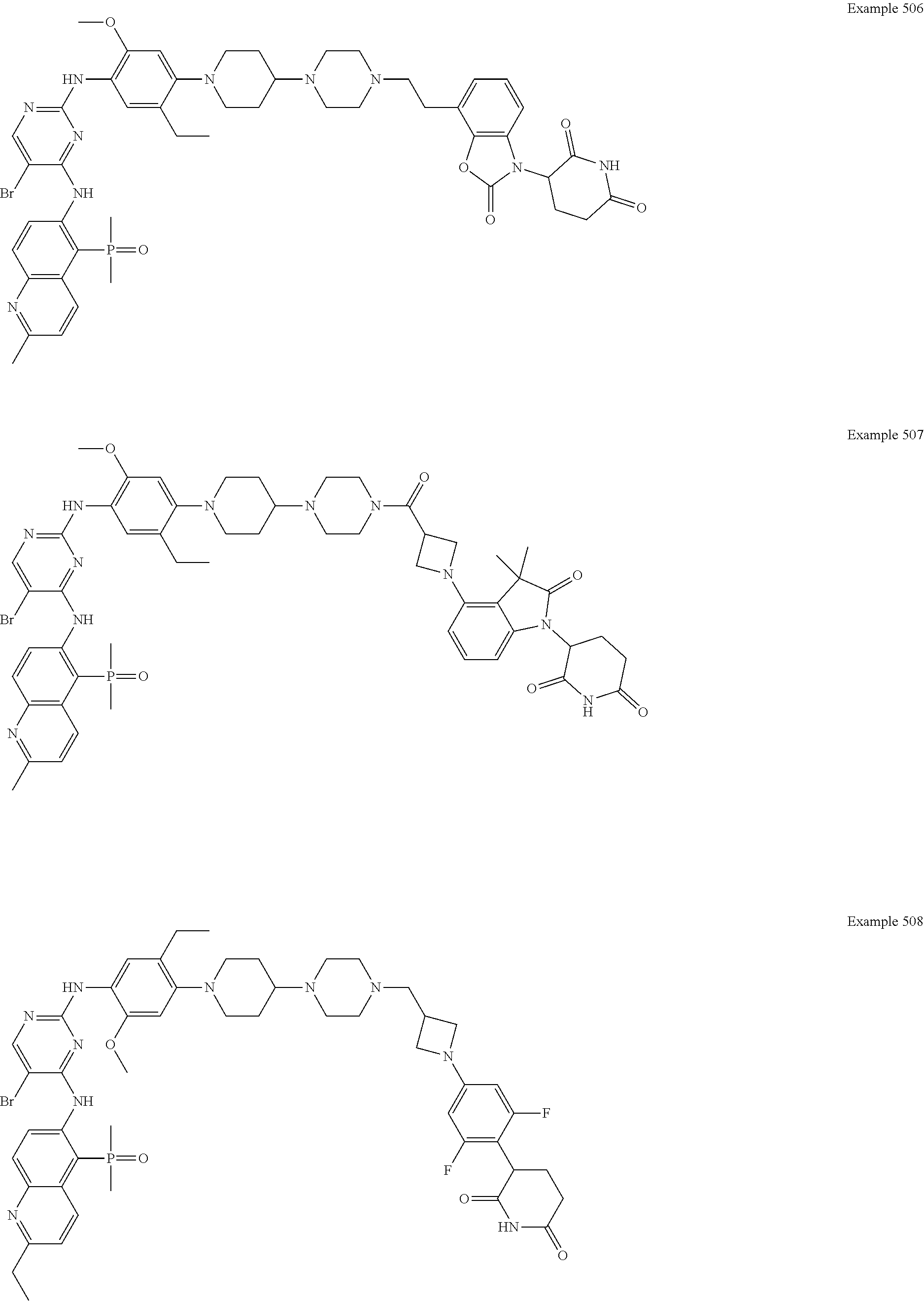 [SMILES: CCc1cc(Nc2ncc(Br)c(Nc3ccc4nc(C)ccc4c3P(C)(C)=O)n2)c(OC)cc1N1CCC(N2CCN(CCc3cccc4c3oc(=O)n4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4C(C)(C)C(=O)N5C4CCC(=O)NC4=O)C3)CC2)CC1 ; CCc1ccc2c(P(C)(C)=O)c(Nc3nc(Nc4cc(CC)c(N5CCC(N6CCN(CC7CN(c8cc(F)c(C9CCC(=O)NC9=O)c(F)c8)C7)CC6)CC5)cc4OC)ncc3Br)ccc2n1]

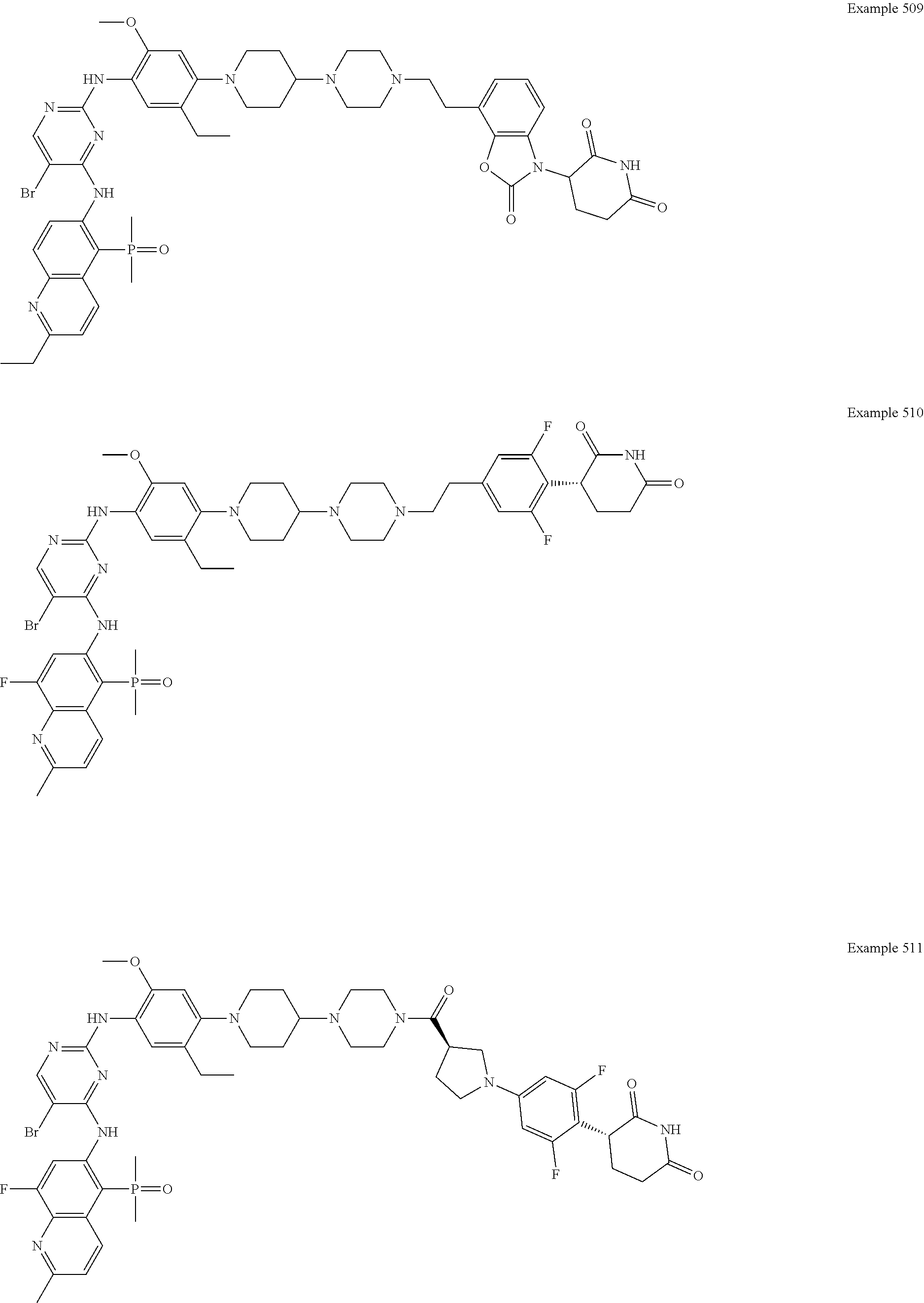 [SMILES: CCc1ccc2c(P(C)(C)=O)c(Nc3nc(Nc4cc(CC)c(N5CCC(N6CCN(CCc7cccc8c7oc(=O)n8C7CCC(=O)NC7=O)CC6)CC5)cc4OC)ncc3Br)ccc2n1 ; CCc1cc(Nc2ncc(Br)c(Nc3cc(F)c4nc(C)ccc4c3P(C)(C)=O)n2)c(OC)cc1N1CCC(N2CCN(CCc3cc(F)c([C@H]4CCC(=O)NC4=O)c(F)c3)CC2)CC1 ; CCc1cc(Nc2ncc(Br)c(Nc3cc(F)c4nc(C)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

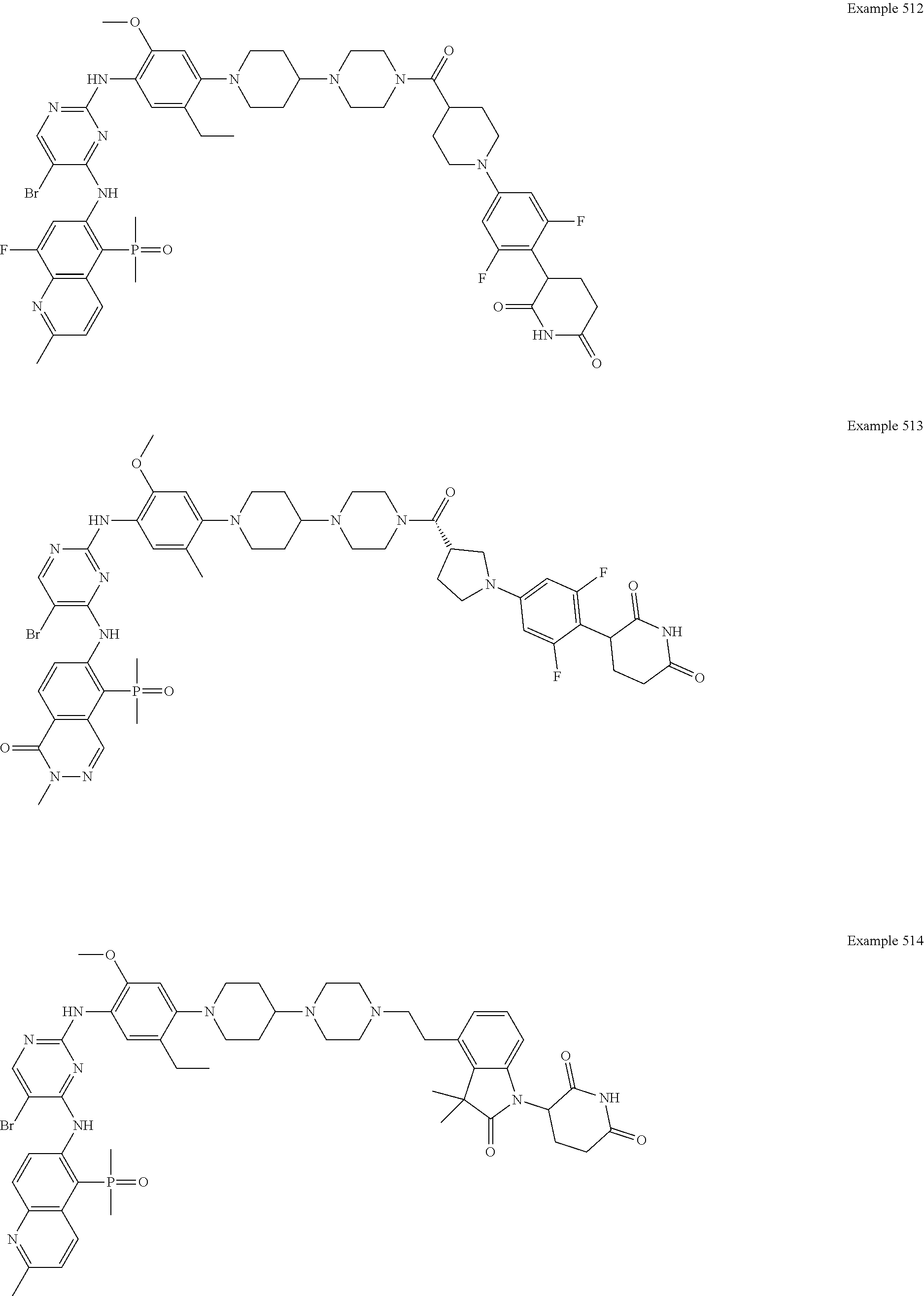

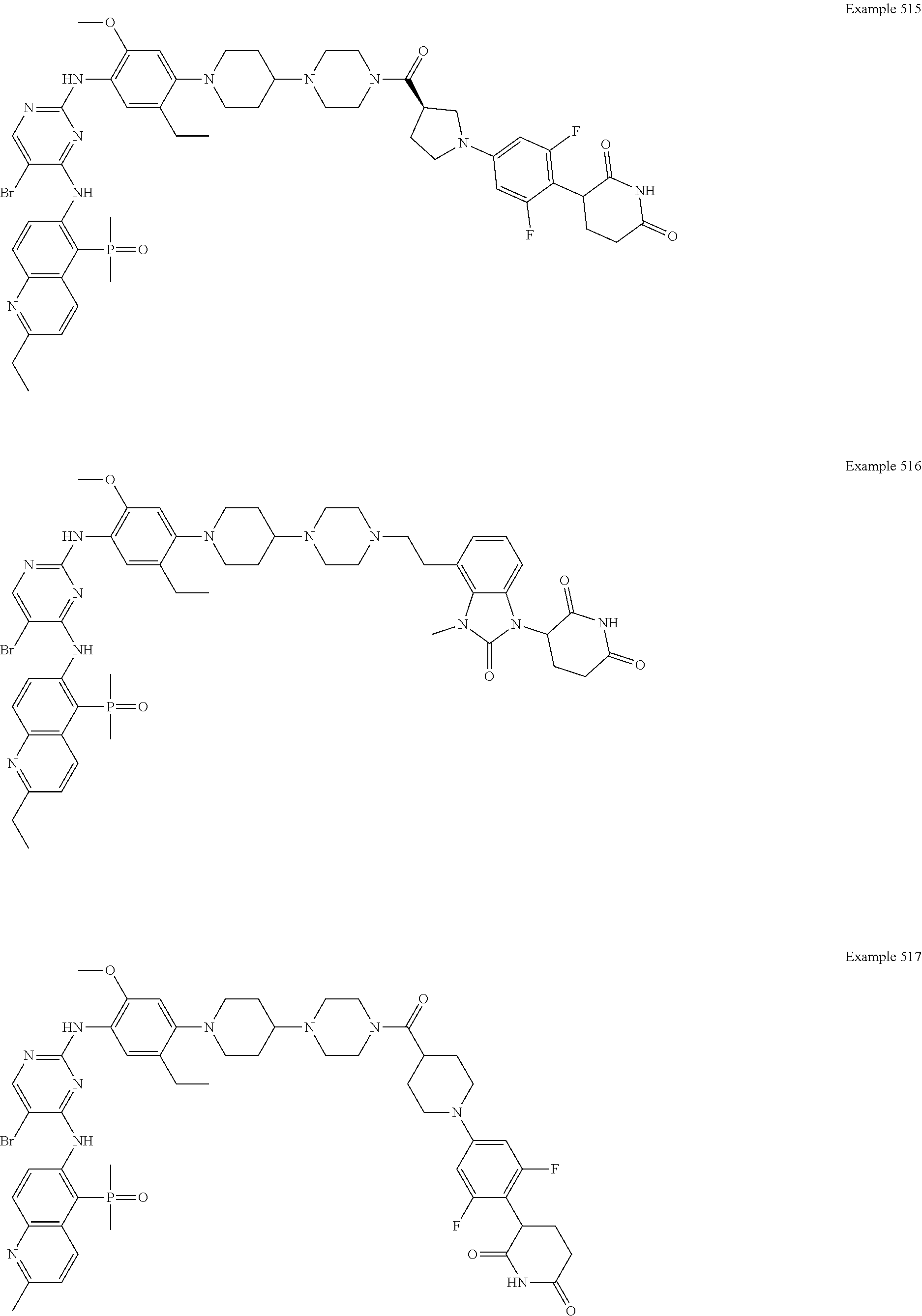 [SMILES: CCc1ccc2c(P(C)(C)=O)c(Nc3nc(Nc4cc(CC)c(N5CCC(N6CCN(C(=O)[C@@H]7CCN(c8cc(F)c(C9CCC(=O)NC9=O)c(F)c8)C7)CC6)CC5)cc4OC)ncc3Br)ccc2n1 ; CCc1ccc2c(P(C)(C)=O)c(Nc3nc(Nc4cc(CC)c(N5CCC(N6CCN(CCc7cccc8c7n(C)c(=O)n8C7CCC(=O)NC7=O)CC6)CC5)cc4OC)ncc3Br)ccc2n1 ; CCc1cc(Nc2ncc(Br)c(Nc3ccc4nc(C)ccc4c3P(C)(C)=O)n2)c(OC)cc1N1CCC(N2CCN(C(=O)C3CCN(c4cc(F)c(C5CCC(=O)NC5=O)c(F)c4)CC3)CC2)CC1]

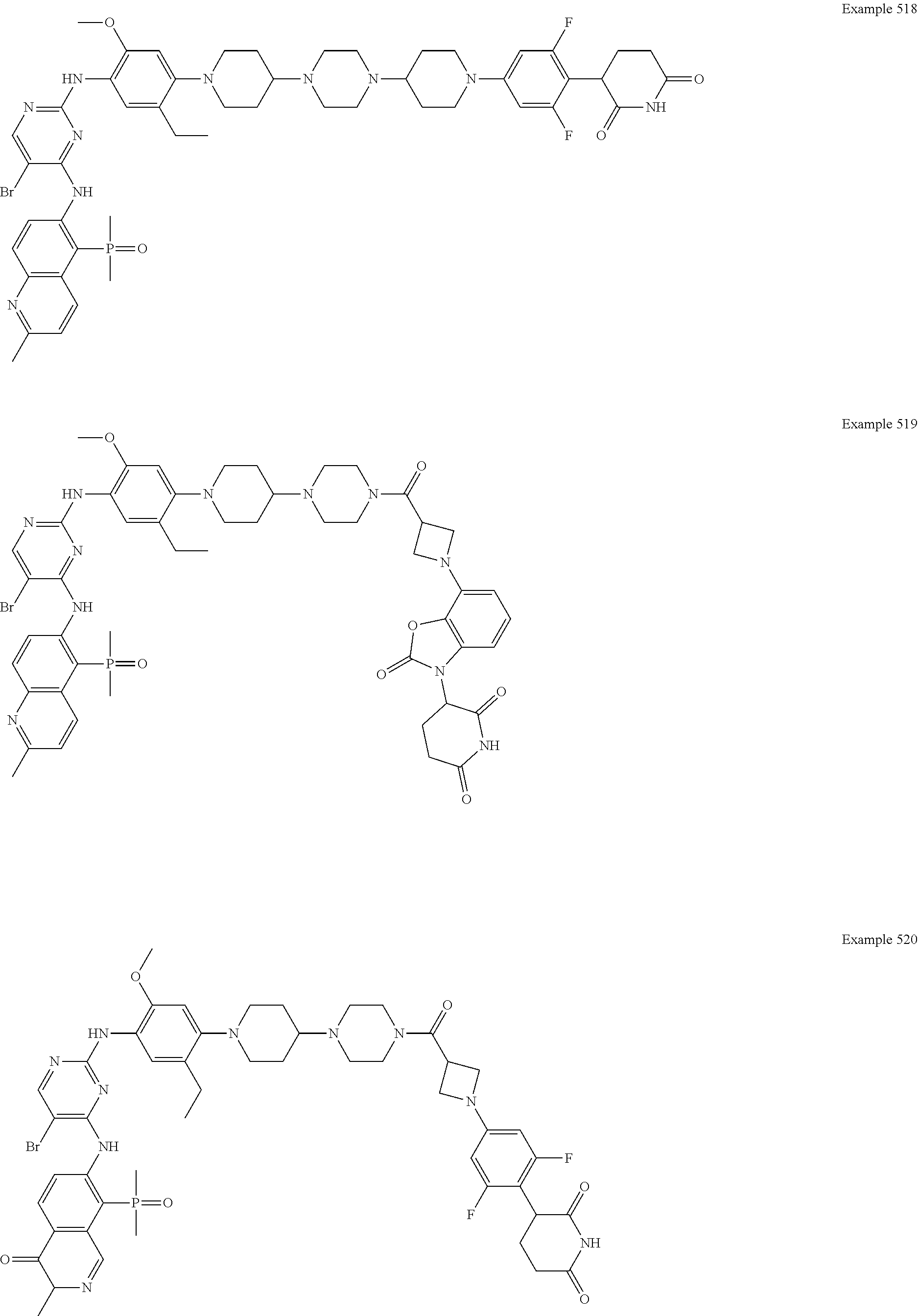 [SMILES: CCc1cc(Nc2ncc(Br)c(Nc3ccc4nc(C)ccc4c3P(C)(C)=O)n2)c(OC)cc1N1CCC(N2CCN(C3CCN(c4cc(F)c(C5CCC(=O)NC5=O)c(F)c4)CC3)CC2)CC1 ; CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4oc(=O)n5C4CCC(=O)NC4=O)C3)CC2)CC1 ; CCc1cc(Nc2ncc(Br)c(Nc3ccc4c(c3P(C)(C)=O)C=NC(C)C4=O)n2)c(OC)cc1N1CCC(N2CCN(C(=O)C3CN(c4cc(F)c(C5CCC(=O)NC5=O)c(F)c4)C3)CC2)CC1]

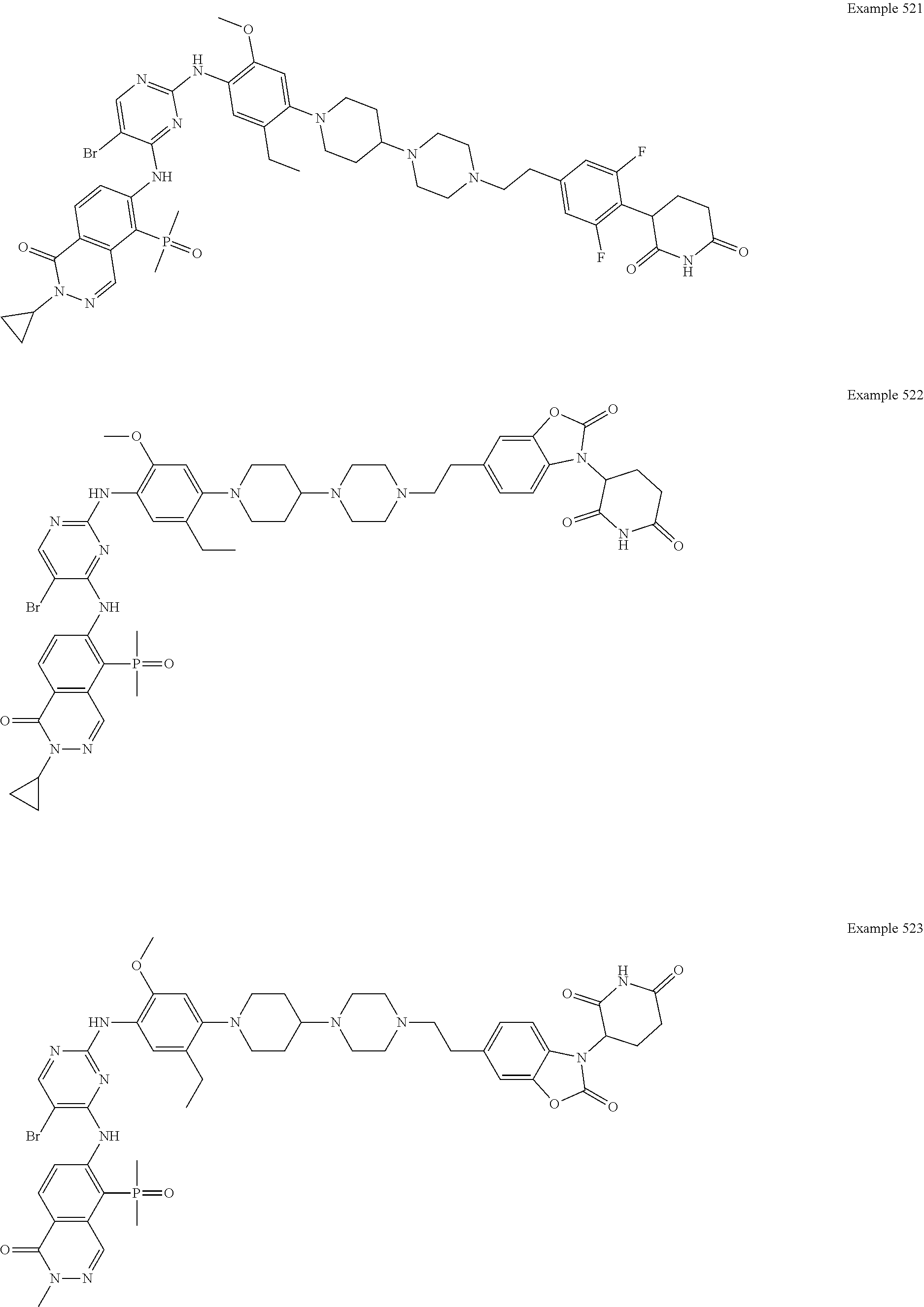 [SMILES: CCc1cc(Nc2ncc(Br)c(Nc3ccc4c(=O)n(C5CC5)ncc4c3P(C)(C)=O)n2)c(OC)cc1N1CCC(N2CCN(CCc3cc(F)c(C4CCC(=O)NC4=O)c(F)c3)CC2)CC1 ; CCc1cc(Nc2ncc(Br)c(Nc3ccc4c(=O)n(C5CC5)ncc4c3P(C)(C)=O)n2)c(OC)cc1N1CCC(N2CCN(CCc3ccc4c(c3)oc(=O)n4C3CCC(=O)NC3=O)CC2)CC1 ; CCc1cc(Nc2ncc(Br)c(Nc3ccc4c(=O)n(C)ncc4c3P(C)(C)=O)n2)c(OC)cc1N1CCC(N2CCN(CCc3ccc4c(c3)oc(=O)n4C3CCC(=O)NC3=O)CC2)CC1]

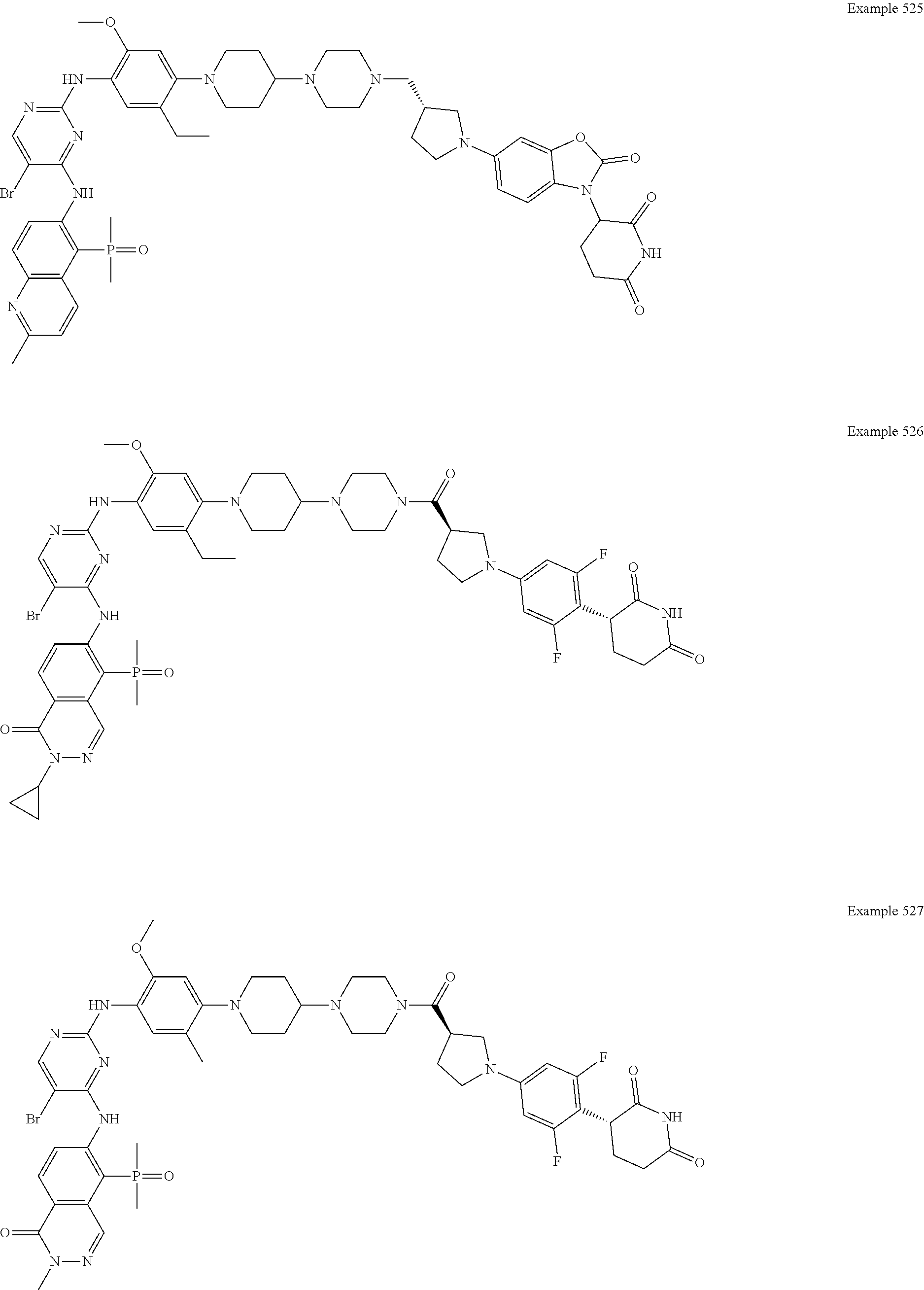 [SMILES: CCc1cc(Nc2ncc(Br)c(Nc3ccc4nc(C)ccc4c3P(C)(C)=O)n2)c(OC)cc1N1CCC(N2CCN(C[C@H]3CCN(c4ccc5c(c4)oc(=O)n5C4CCC(=O)NC4=O)C3)CC2)CC1 ; CCc1cc(Nc2ncc(Br)c(Nc3ccc4c(=O)n(C5CC5)ncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; COc1cc(N2CCC(N3CCN(C(=O)[C@@H]4CCN(c5cc(F)c([C@H]6CCC(=O)NC6=O)c(F)c5)C4)CC3)CC2)c(C)cc1Nc1ncc(Br)c(Nc2ccc3c(=O)n(C)ncc3c2P(C)(C)=O)n1]

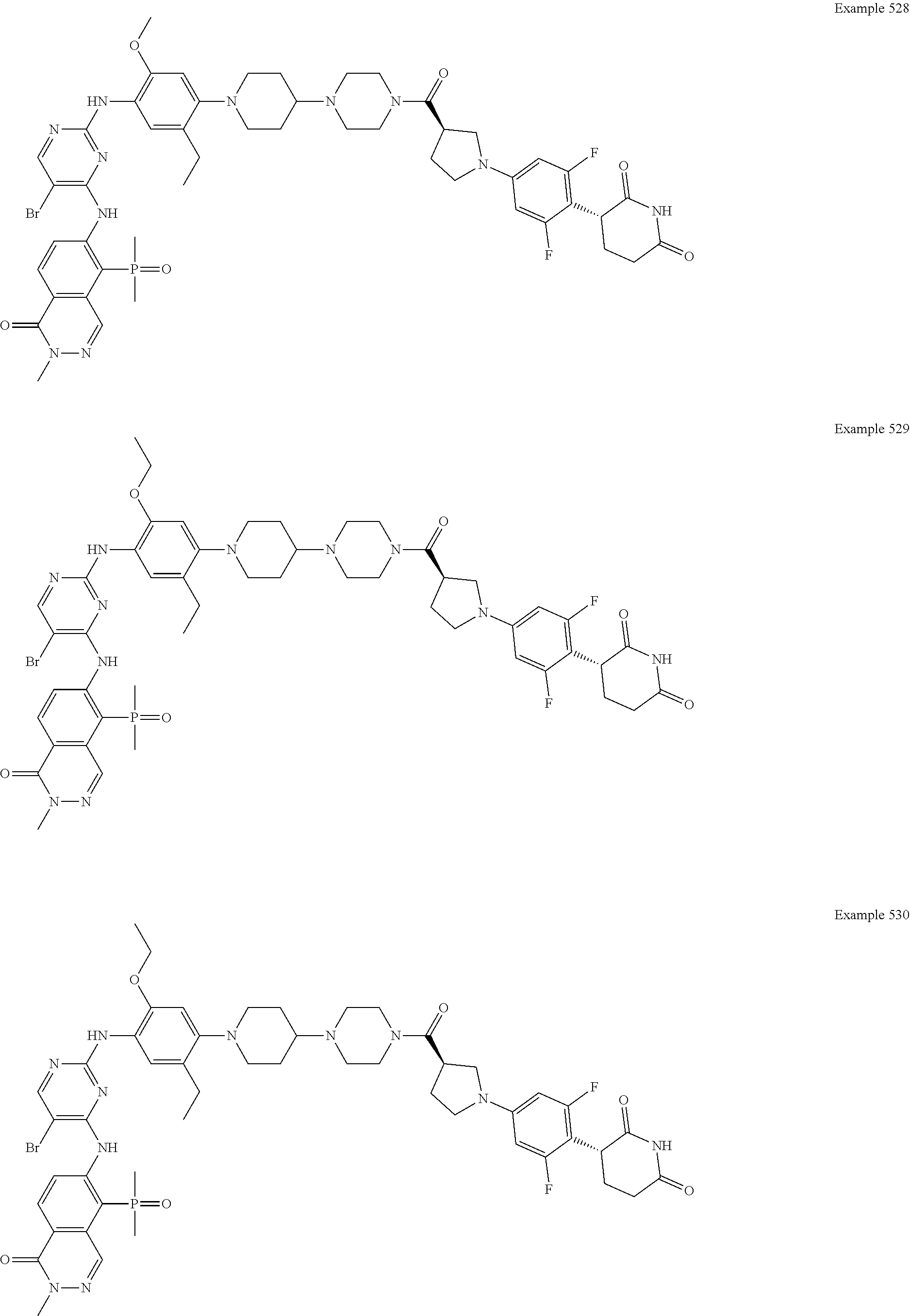 [SMILES: CCc1cc(Nc2ncc(Br)c(Nc3ccc4c(=O)n(C)ncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; CCOc1cc(N2CCC(N3CCN(C(=O)[C@@H]4CCN(c5cc(F)c([C@H]6CCC(=O)NC6=O)c(F)c5)C4)CC3)CC2)c(CC)cc1Nc1ncc(Br)c(Nc2ccc3c(=O)n(C)ncc3c2P(C)(C)=O)n1 ; CCOc1cc(N2CCC(N3CCN(C(=O)[C@@H]4CCN(c5cc(F)c([C@H]6CCC(=O)NC6=O)c(F)c5)C4)CC3)CC2)c(CC)cc1Nc1ncc(Br)c(Nc2ccc3c(=O)n(C)ncc3c2P(C)(C)=O)n1]

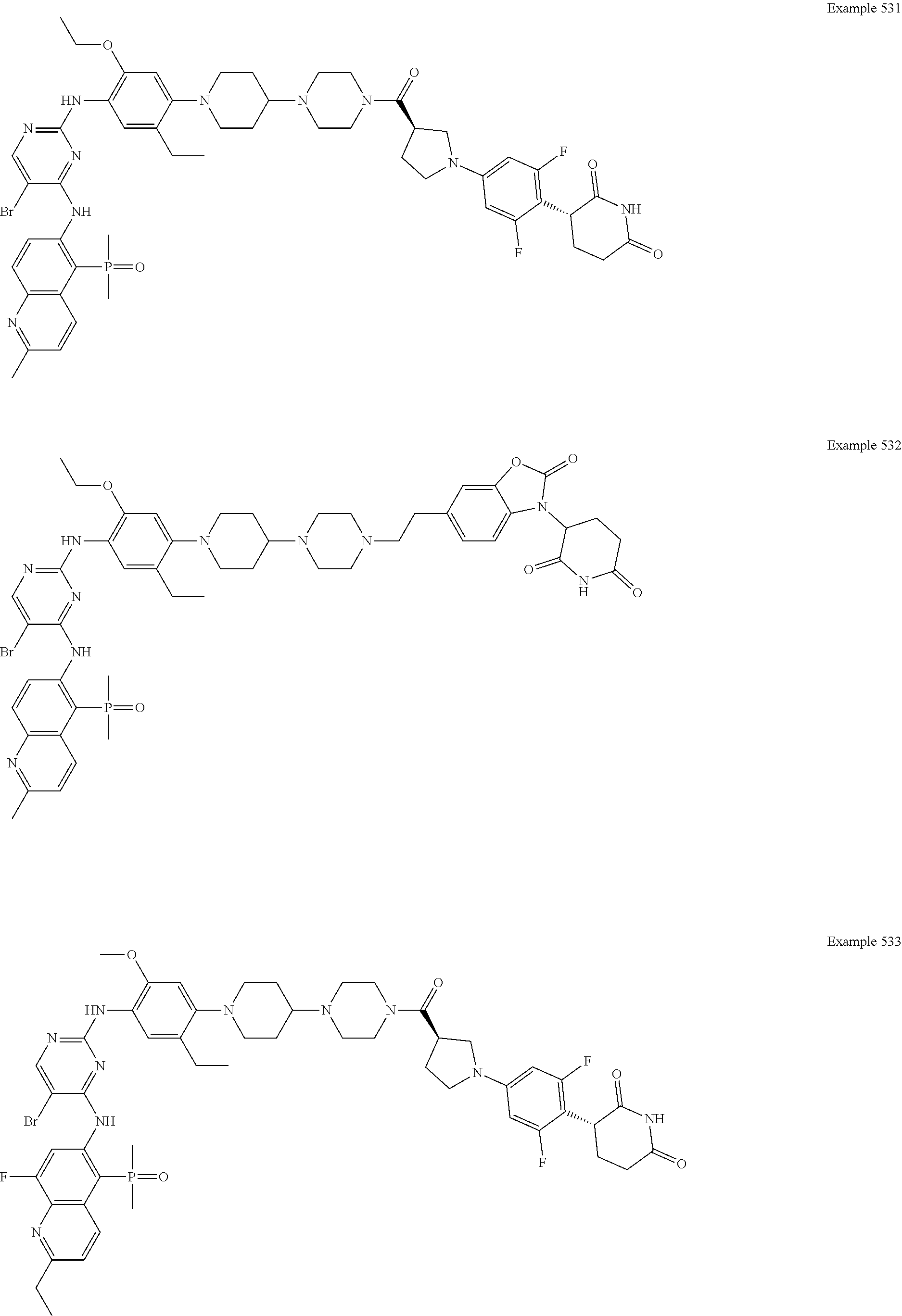 [SMILES: CCOc1cc(N2CCC(N3CCN(C(=O)[C@@H]4CCN(c5cc(F)c([C@H]6CCC(=O)NC6=O)c(F)c5)C4)CC3)CC2)c(CC)cc1Nc1ncc(Br)c(Nc2ccc3nc(C)ccc3c2P(C)(C)=O)n1 ; CCOc1cc(N2CCC(N3CCN(CCc4ccc5c(c4)oc(=O)n5C4CCC(=O)NC4=O)CC3)CC2)c(CC)cc1Nc1ncc(Br)c(Nc2ccc3nc(C)ccc3c2P(C)(C)=O)n1 ; CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)cc(F)c2n1]

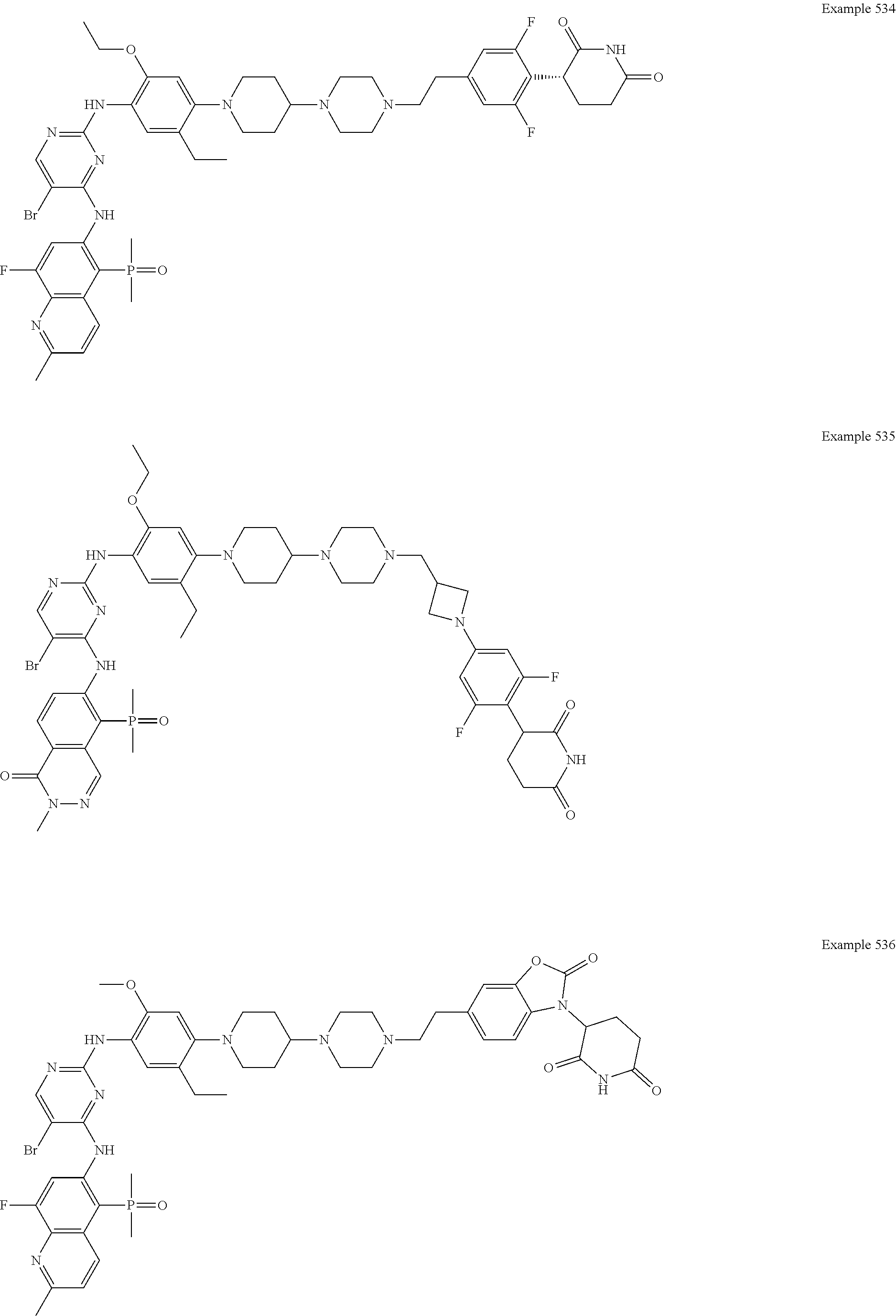 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2cc(F)c3nc(C)ccc3c2P(C)(C)=O)n1 ; CCOc1cc(N2CCC(N3CCN(CC4CN(c5cc(F)c(C6CCC(=O)NC6=O)c(F)c5)C4)CC3)CC2)c(CC)cc1Nc1ncc(Br)c(Nc2ccc3c(=O)n(C)ncc3c2P(C)(C)=O)n1 ; CCc1cc(Nc2ncc(Br)c(Nc3cc(F)c4nc(C)ccc4c3P(C)(C)=O)n2)c(OC)cc1N1CCC(N2CCN(CCc3ccc4c(c3)oc(=O)n4C3CCC(=O)NC3=O)CC2)CC1]

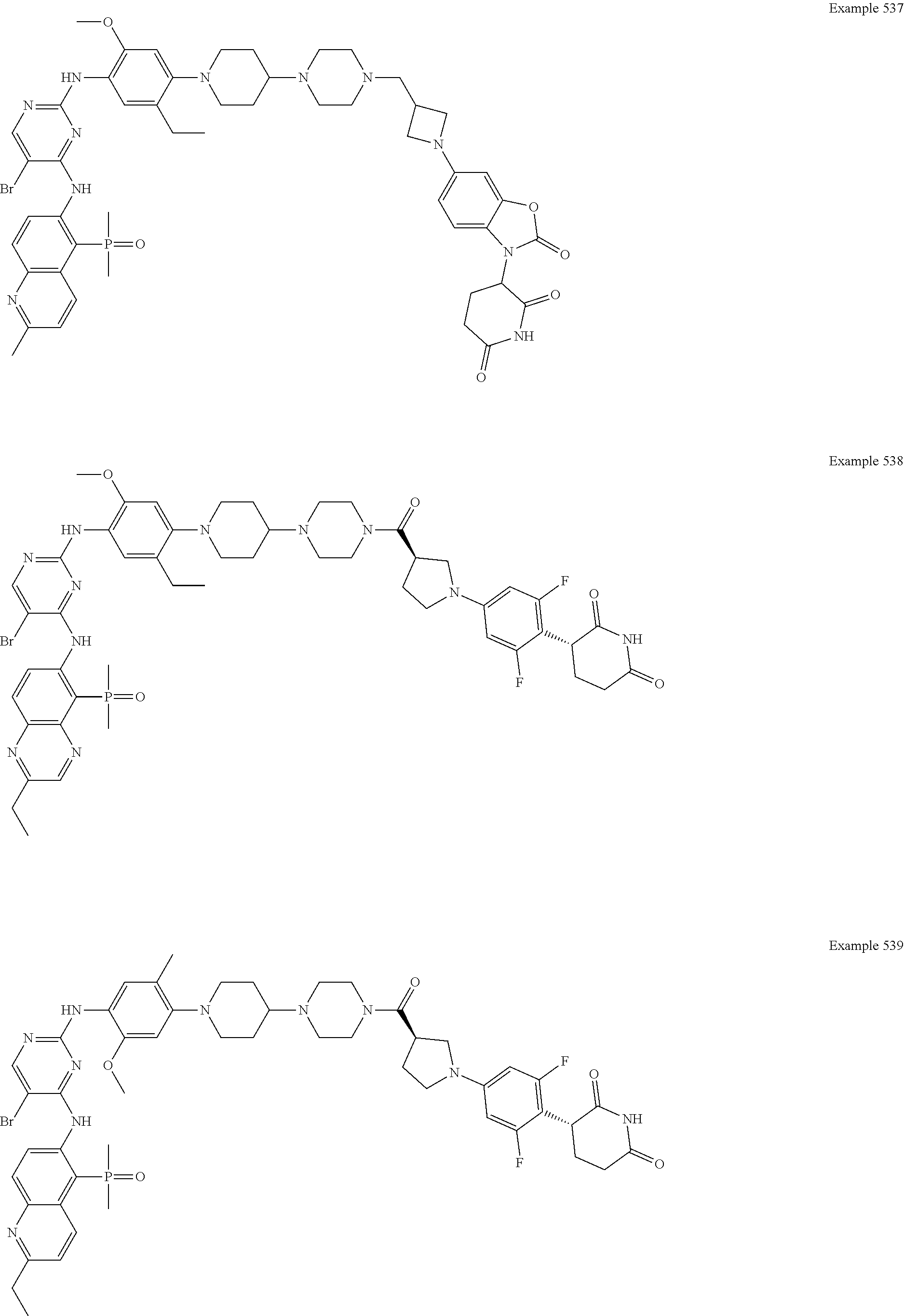 [SMILES: CCc1cc(Nc2ncc(Br)c(Nc3ccc4nc(C)ccc4c3P(C)(C)=O)n2)c(OC)cc1N1CCC(N2CCN(CC3CN(c4ccc5c(c4)oc(=O)n5C4CCC(=O)NC4=O)C3)CC2)CC1 ; CCc1cnc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1 ; CCc1ccc2c(P(C)(C)=O)c(Nc3nc(Nc4cc(C)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1]

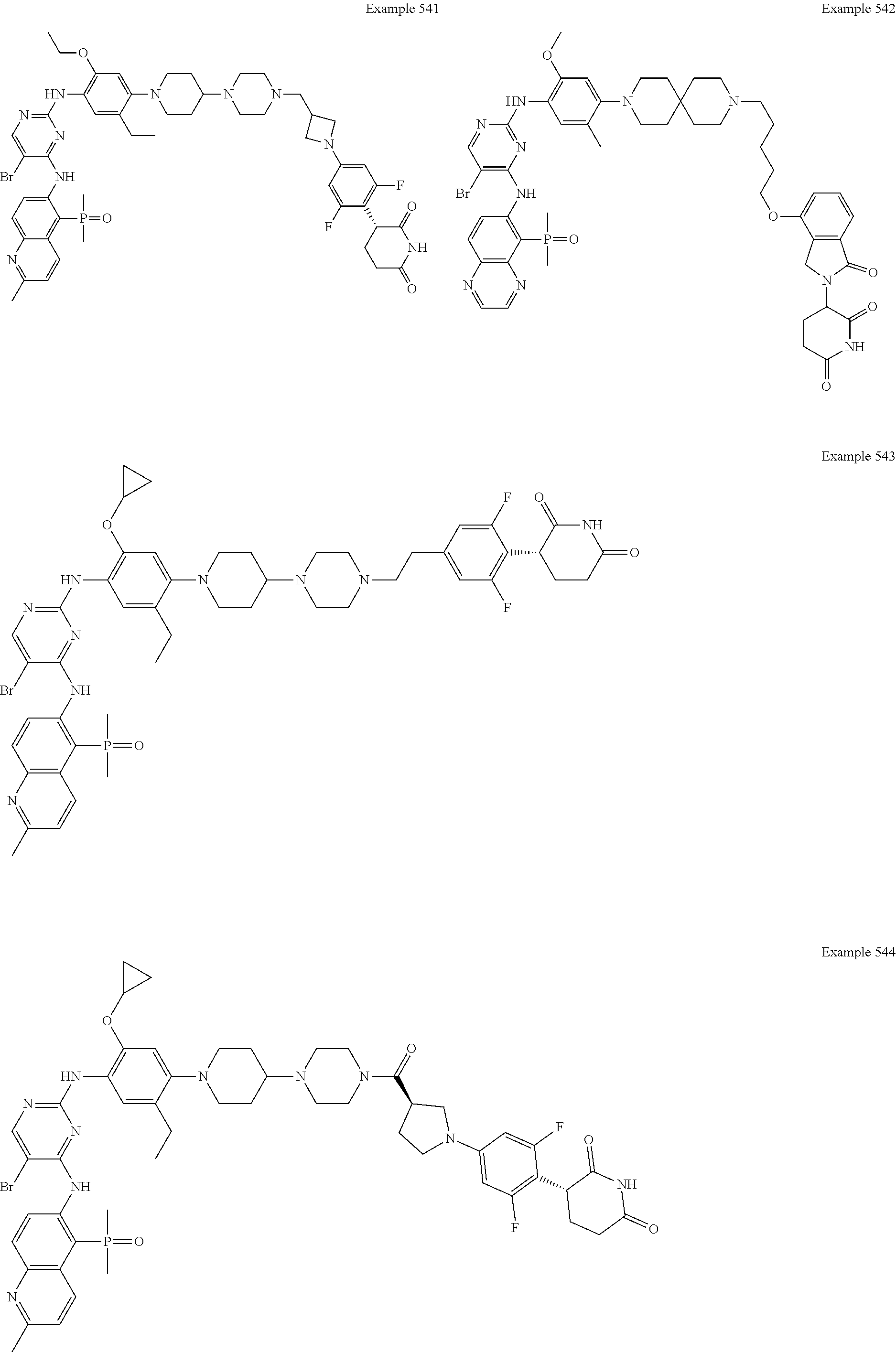 [SMILES: CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3nc(C)ccc3c2P(C)(C)=O)n1 ; COc1cc(N2CCC3(CCN(CCCCCOc4cccc5c4CN(C4CCC(=O)NC4=O)C5=O)CC3)CC2)c(C)cc1Nc1ncc(Br)c(Nc2ccc3nccnc3c2P(C)(C)=O)n1 ; CCc1cc(Nc2ncc(Br)c(Nc3ccc4nc(C)ccc4c3P(C)(C)=O)n2)c(OC2CC2)cc1N1CCC(N2CCN(CCc3cc(F)c([C@H]4CCC(=O)NC4=O)c(F)c3)CC2)CC1 ; CCc1cc(Nc2ncc(Br)c(Nc3ccc4nc(C)ccc4c3P(C)(C)=O)n2)c(OC2CC2)cc1N1CCC(N2CCN(C(=O)[C@@H]3CCN(c4cc(F)c([C@H]5CCC(=O)NC5=O)c(F)c4)C3)CC2)CC1]

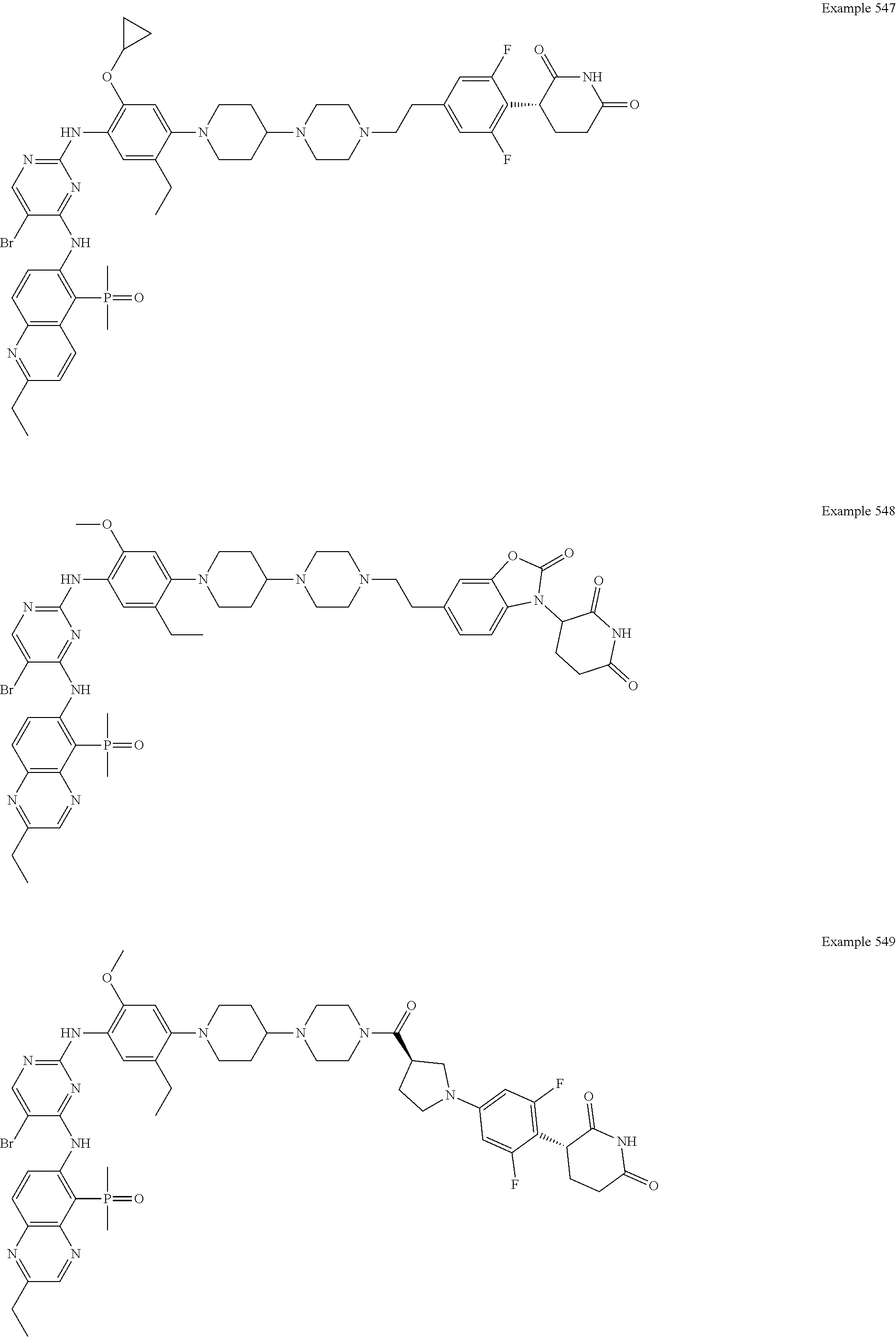 [SMILES: CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC4CC4)ncc3Br)ccc2n1 ; CCc1cnc2c(P(C)(C)=O)c(Nc3nc(Nc4cc(CC)c(N5CCC(N6CCN(CCc7ccc8c(c7)oc(=O)n8C7CCC(=O)NC7=O)CC6)CC5)cc4OC)ncc3Br)ccc2n1 ; CCc1cnc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1]

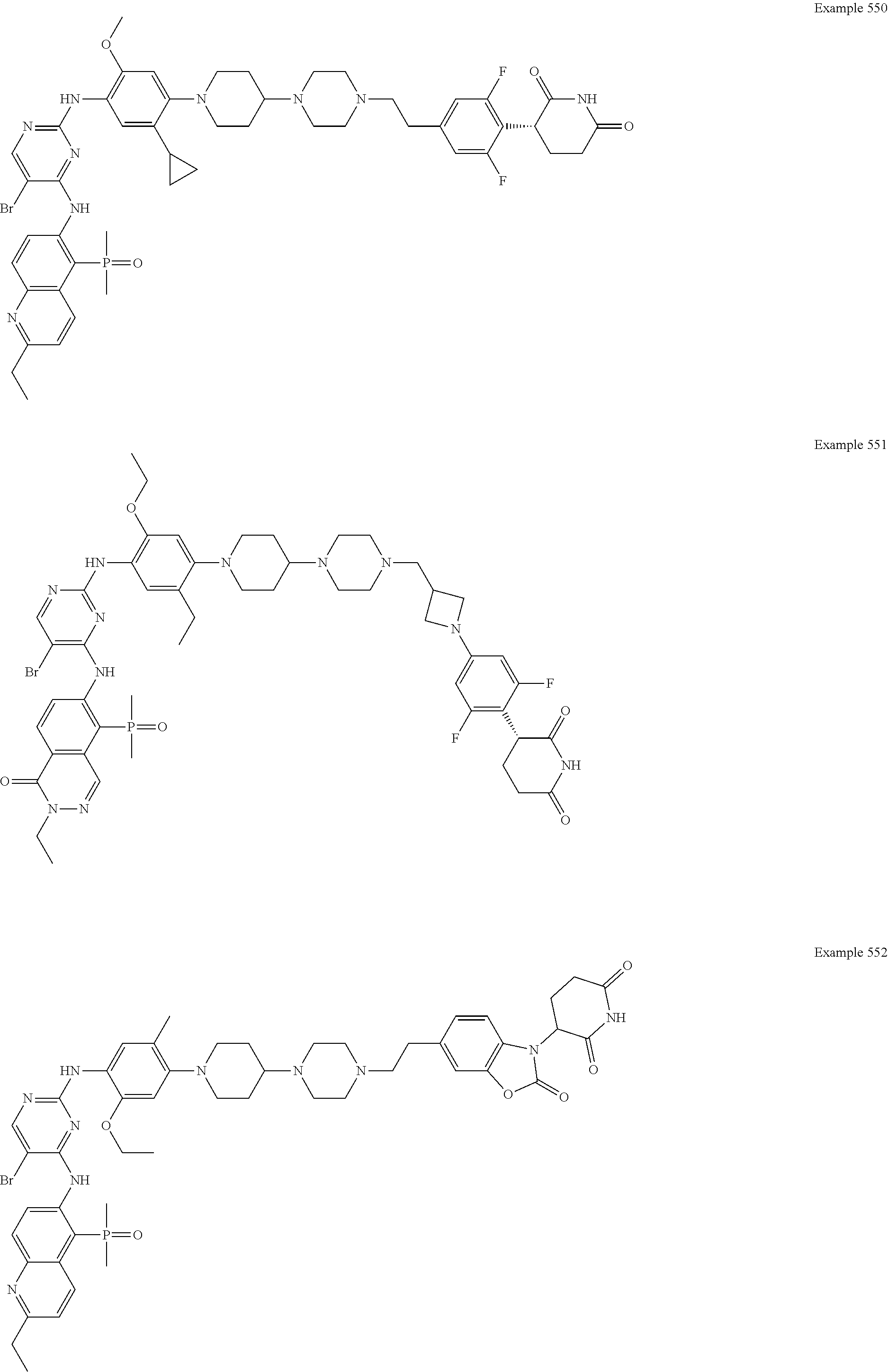 [SMILES: CCc1ccc2c(P(C)(C)=O)c(Nc3nc(Nc4cc(C5CC5)c(N5CCC(N6CCN(CCc7cc(F)c([C@H]8CCC(=O)NC8=O)c(F)c7)CC6)CC5)cc4OC)ncc3Br)ccc2n1 ; CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCc4ccc5c(c4)oc(=O)n5C4CCC(=O)NC4=O)CC3)CC2)c(C)cc1Nc1ncc(Br)c(Nc2ccc3nc(CC)ccc3c2P(C)(C)=O)n1]

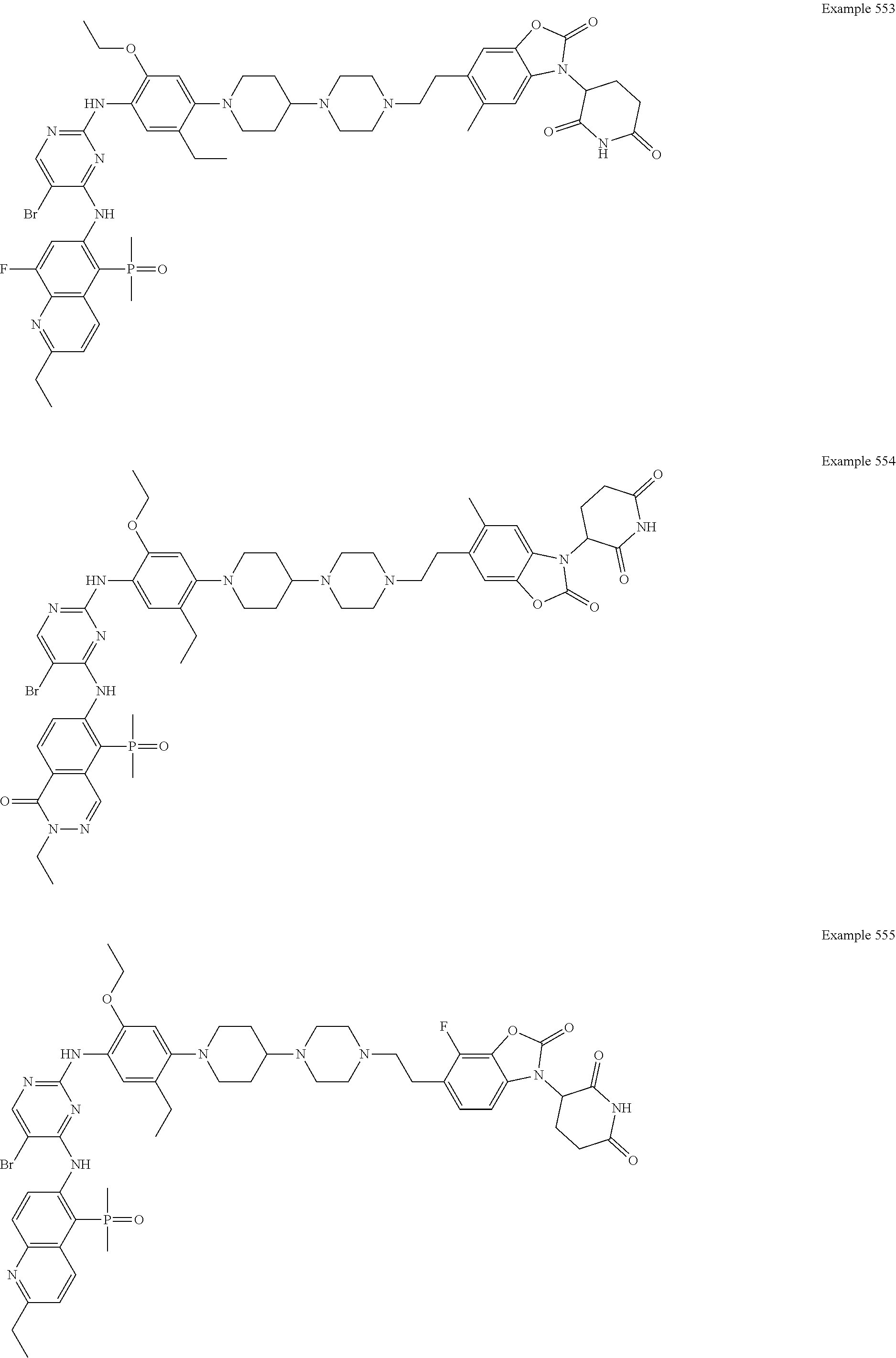 [SMILES: CCOc1cc(N2CCC(N3CCN(CCc4cc5oc(=O)n(C6CCC(=O)NC6=O)c5cc4C)CC3)CC2)c(CC)cc1Nc1ncc(Br)c(Nc2cc(F)c3nc(CC)ccc3c2P(C)(C)=O)n1 ; CCOc1cc(N2CCC(N3CCN(CCc4cc5oc(=O)n(C6CCC(=O)NC6=O)c5cc4C)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCc4ccc5c(oc(=O)n5C5CCC(=O)NC5=O)c4F)CC3)CC2)c(CC)cc1Nc1ncc(Br)c(Nc2ccc3nc(CC)ccc3c2P(C)(C)=O)n1]

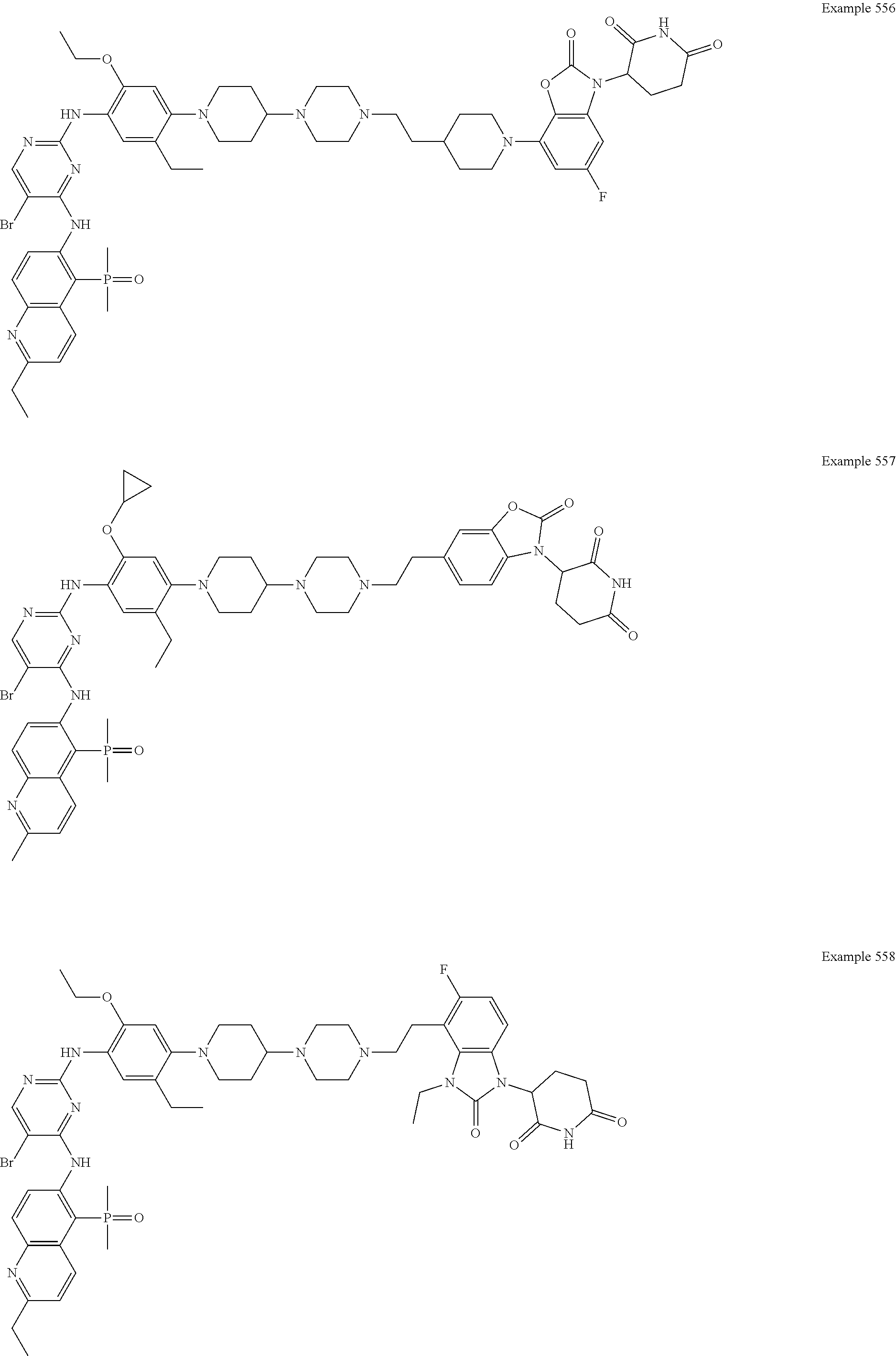 [SMILES: CCOc1cc(N2CCC(N3CCN(CCC4CCN(c5cc(F)cc6c5oc(=O)n6C5CCC(=O)NC5=O)CC4)CC3)CC2)c(CC)cc1Nc1ncc(Br)c(Nc2ccc3nc(CC)ccc3c2P(C)(C)=O)n1 ; CCc1cc(Nc2ncc(Br)c(Nc3ccc4nc(C)ccc4c3P(C)(C)=O)n2)c(OC2CC2)cc1N1CCC(N2CCN(CCc3ccc4c(c3)oc(=O)n4C3CCC(=O)NC3=O)CC2)CC1 ; CCOc1cc(N2CCC(N3CCN(CCc4c(F)ccc5c4n(CC)c(=O)n5C4CCC(=O)NC4=O)CC3)CC2)c(CC)cc1Nc1ncc(Br)c(Nc2ccc3nc(CC)ccc3c2P(C)(C)=O)n1]

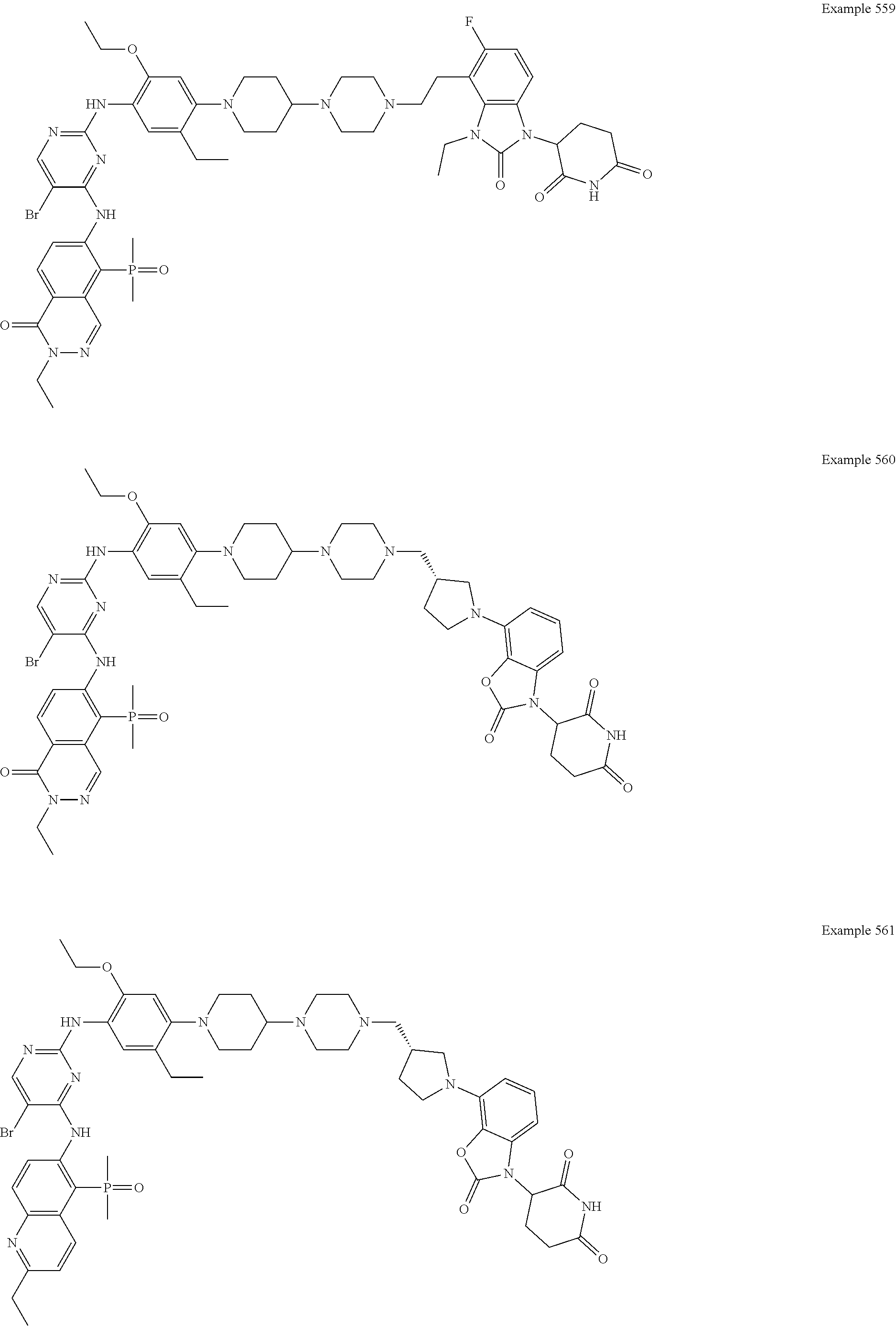 [SMILES: CCOc1cc(N2CCC(N3CCN(CCc4c(F)ccc5c4n(CC)c(=O)n5C4CCC(=O)NC4=O)CC3)CC2)c(CC)cc1Nc1ncc(Br)c(Nc2ccc3c(=O)n(CC)ncc3c2P(C)(C)=O)n1 ; CCOc1cc(N2CCC(N3CCN(C[C@H]4CCN(c5cccc6c5oc(=O)n6C5CCC(=O)NC5=O)C4)CC3)CC2)c(CC)cc1Nc1ncc(Br)c(Nc2ccc3c(=O)n(CC)ncc3c2P(C)(C)=O)n1 ; CCOc1cc(N2CCC(N3CCN(C[C@H]4CCN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

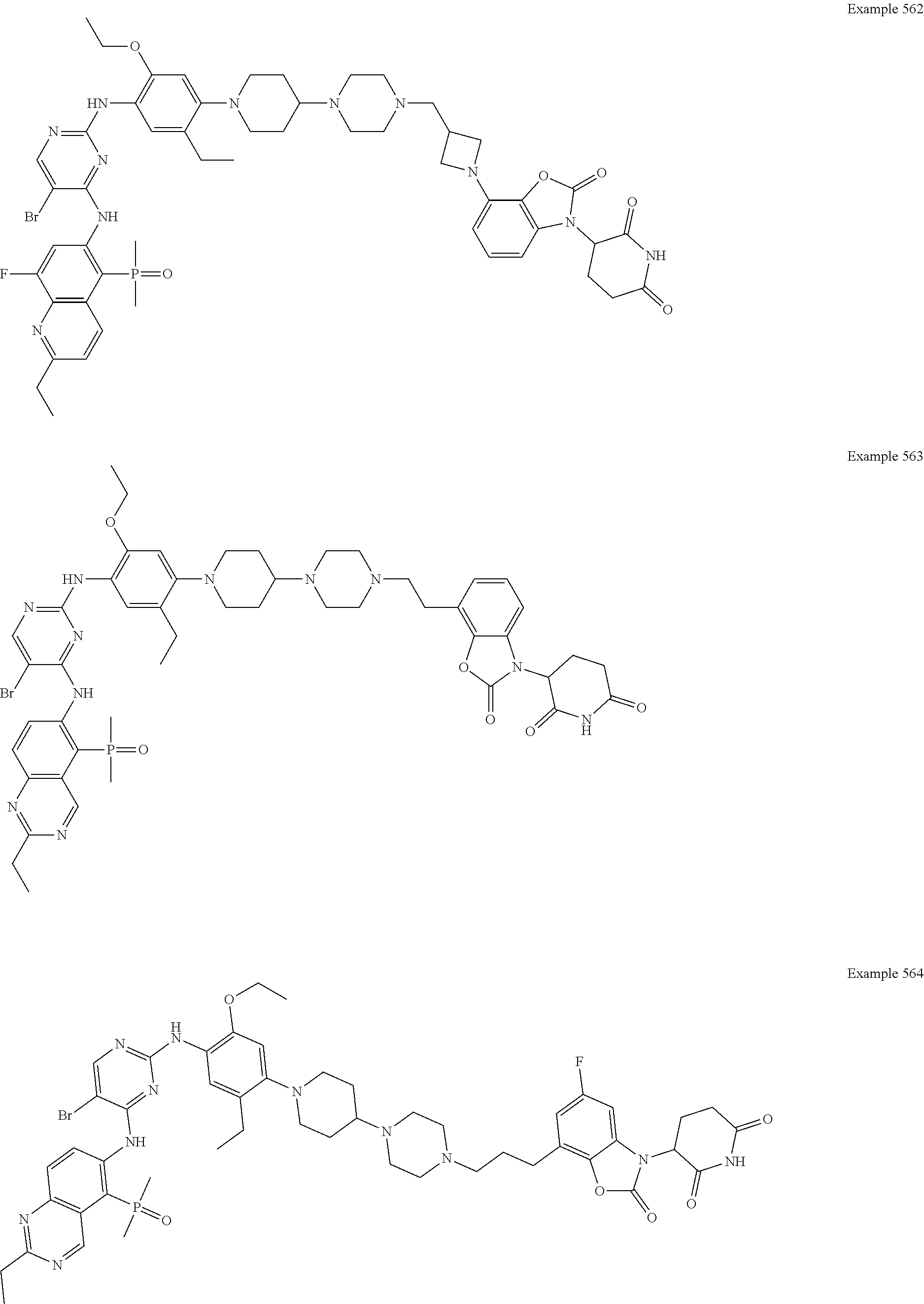 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2cc(F)c3nc(CC)ccc3c2P(C)(C)=O)n1 ; CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3nc(CC)ncc3c2P(C)(C)=O)n1 ; CCOc1cc(N2CCC(N3CCN(CCCc4cc(F)cc5c4oc(=O)n5C4CCC(=O)NC4=O)CC3)CC2)c(CC)cc1Nc1ncc(Br)c(Nc2ccc3nc(CC)ncc3c2P(C)(C)=O)n1]

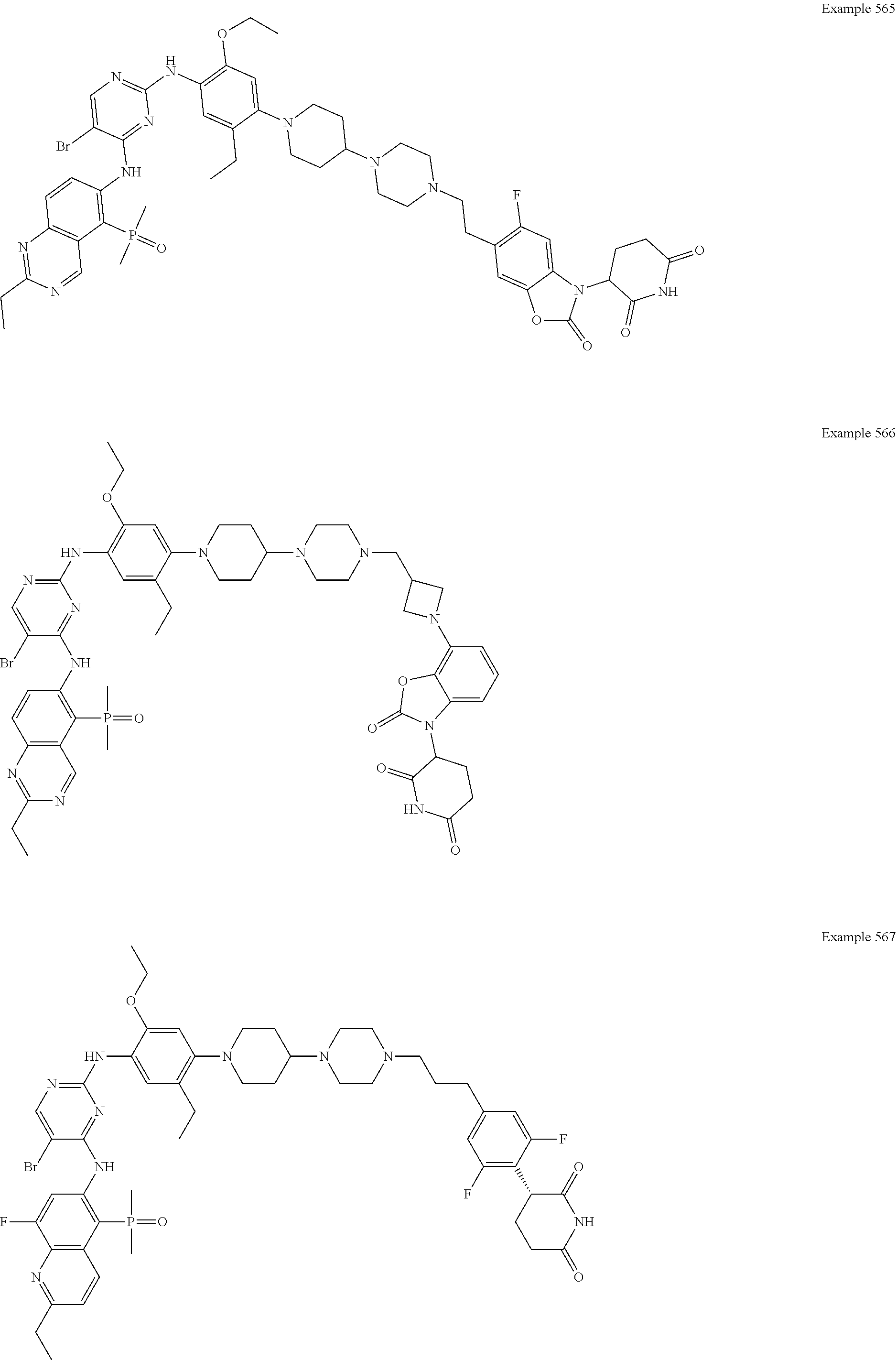 [SMILES: CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2ccc3nc(CC)ncc3c2P(C)(C)=O)n1 ; CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3nc(CC)ncc3c2P(C)(C)=O)n1 ; CCOc1cc(N2CCC(N3CCN(CCCc4cc(F)c([C@H]5CCC(=O)NC5=O)c(F)c4)CC3)CC2)c(CC)cc1Nc1ncc(Br)c(Nc2cc(F)c3nc(CC)ccc3c2P(C)(C)=O)n1]

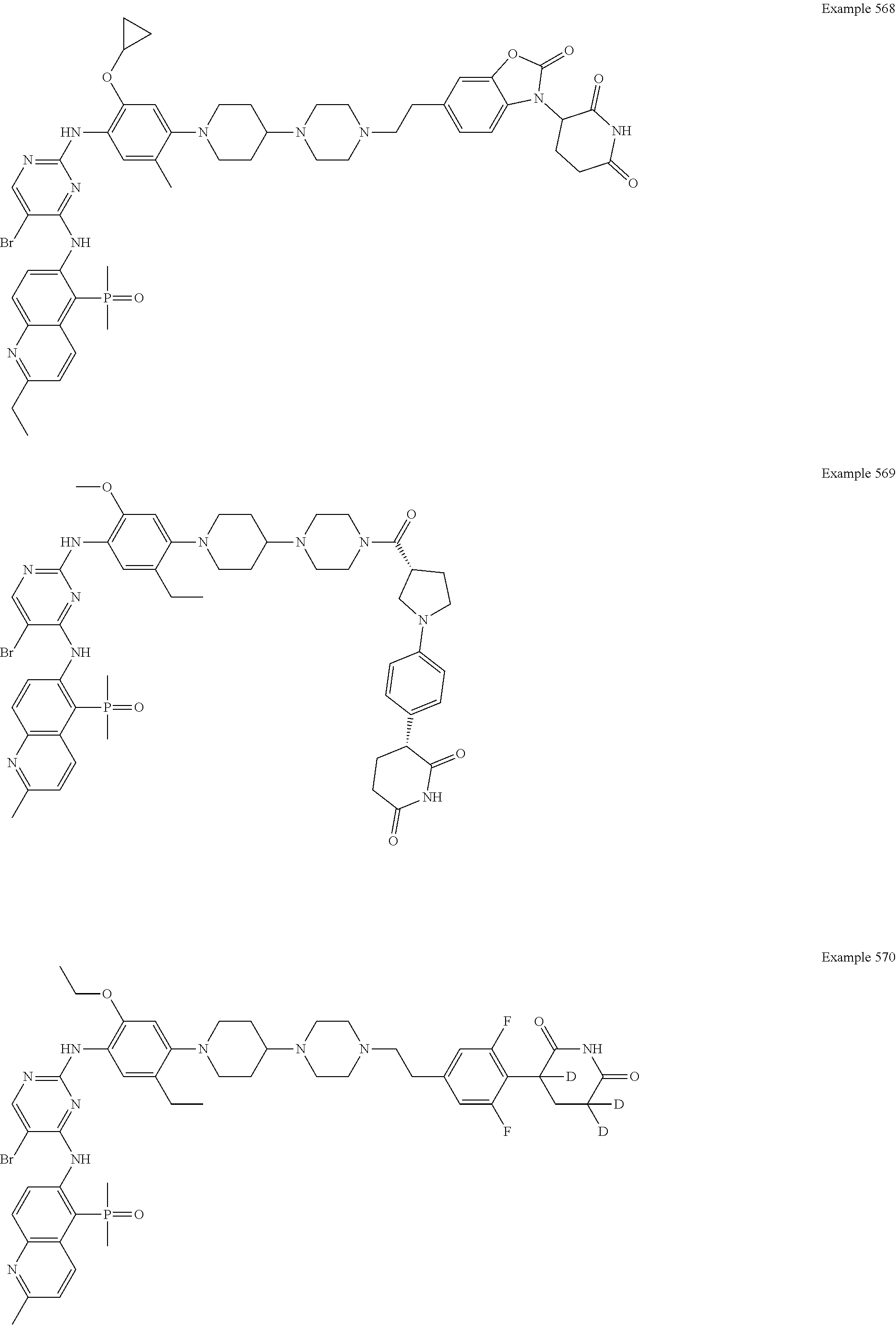

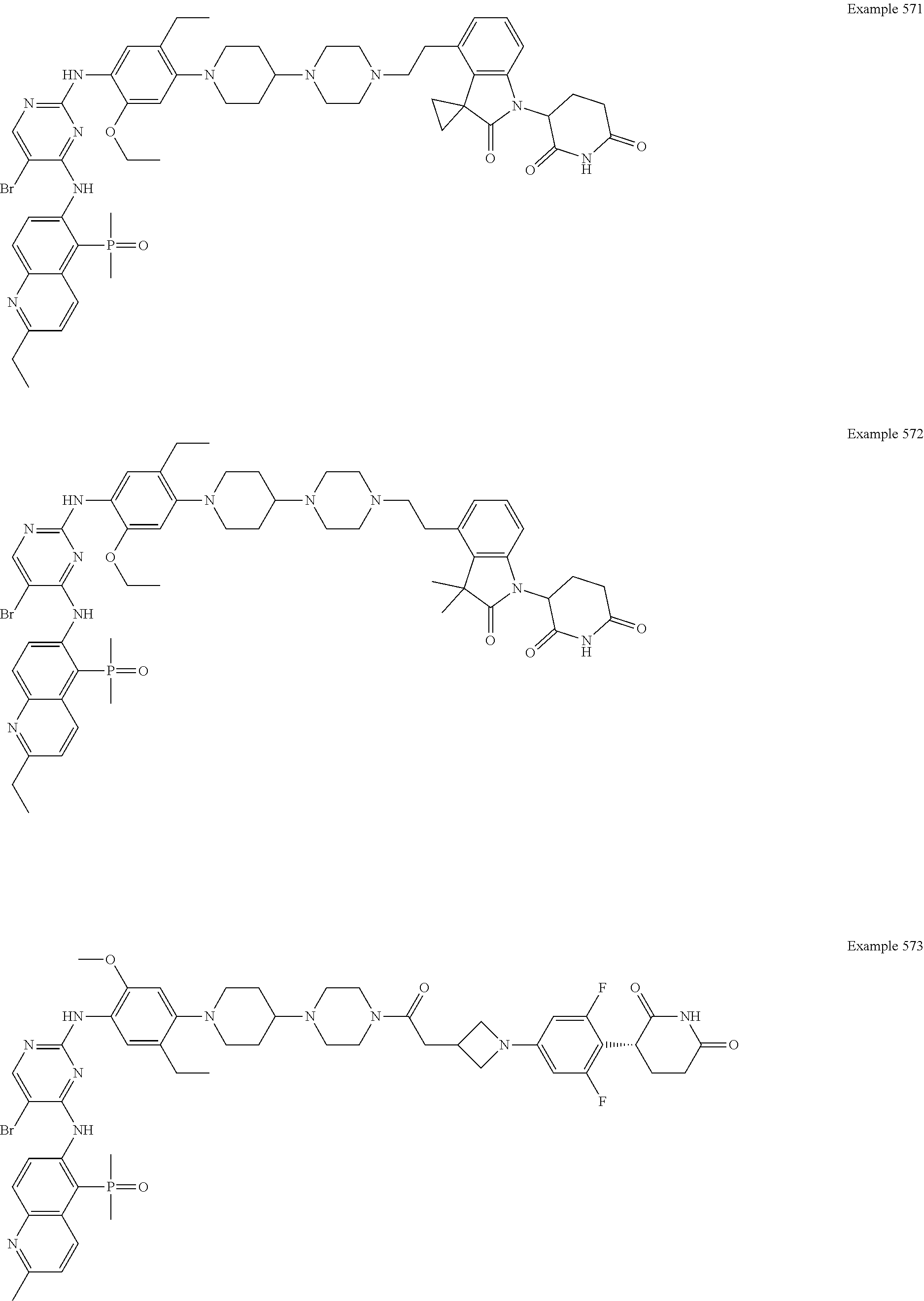 [SMILES: CCOc1cc(N2CCC(N3CCN(CCc4cccc5c4C4(CC4)C(=O)N5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCc4cccc5c4C(C)(C)C(=O)N5C4CCC(=O)NC4=O)CC3)CC2)c(CC)cc1Nc1ncc(Br)c(Nc2ccc3nc(CC)ccc3c2P(C)(C)=O)n1 ; CCc1cc(Nc2ncc(Br)c(Nc3ccc4nc(C)ccc4c3P(C)(C)=O)n2)c(OC)cc1N1CCC(N2CCN(C(=O)CC3CN(c4cc(F)c([C@H]5CCC(=O)NC5=O)c(F)c4)C3)CC2)CC1]

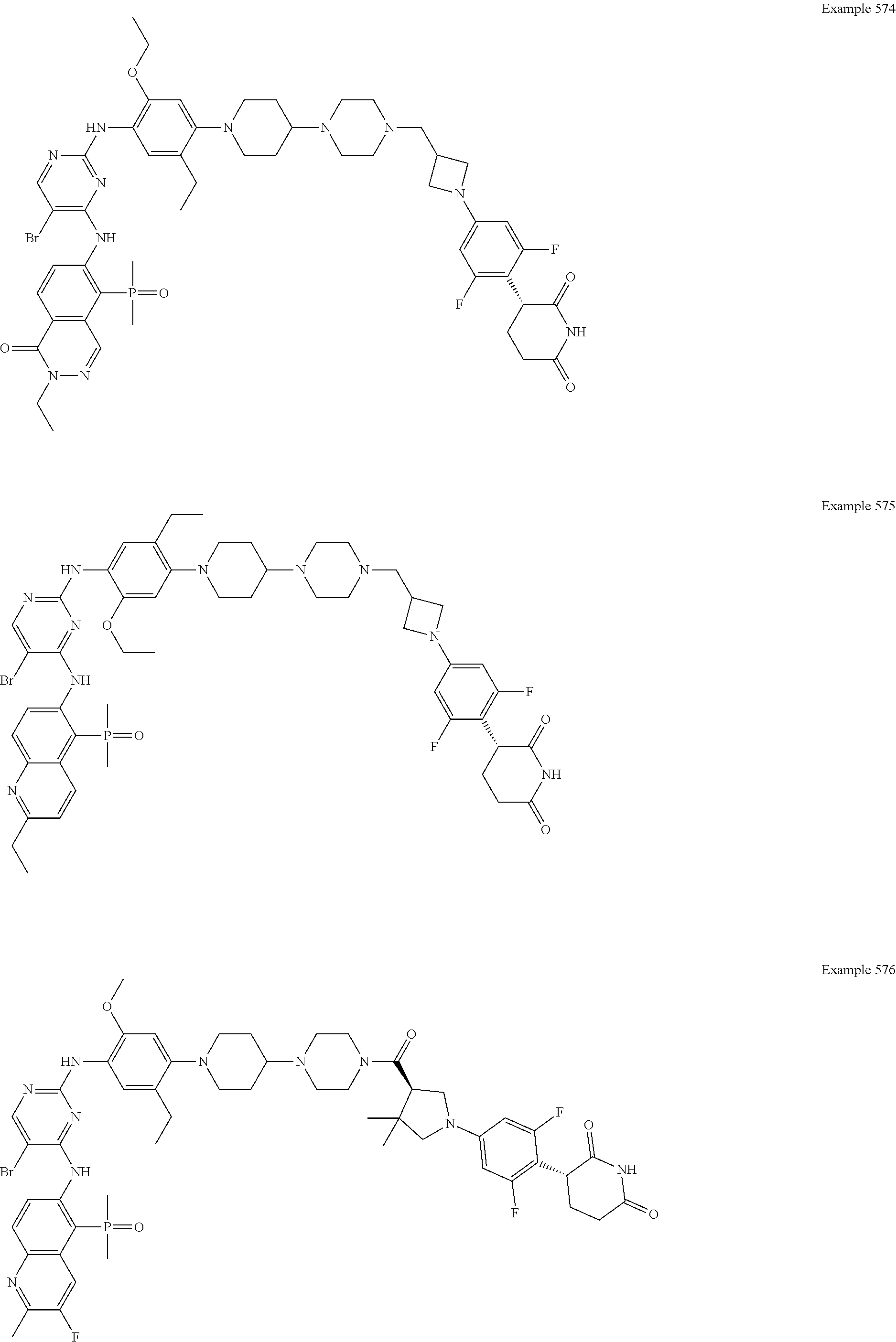 [SMILES: CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3nc(CC)ccc3c2P(C)(C)=O)n1 ; CCc1cc(Nc2ncc(Br)c(Nc3ccc4nc(C)c(F)cc4c3P(C)(C)=O)n2)c(OC)cc1N1CCC(N2CCN(C(=O)[C@H]3CN(c4cc(F)c([C@H]5CCC(=O)NC5=O)c(F)c4)CC3(C)C)CC2)CC1]

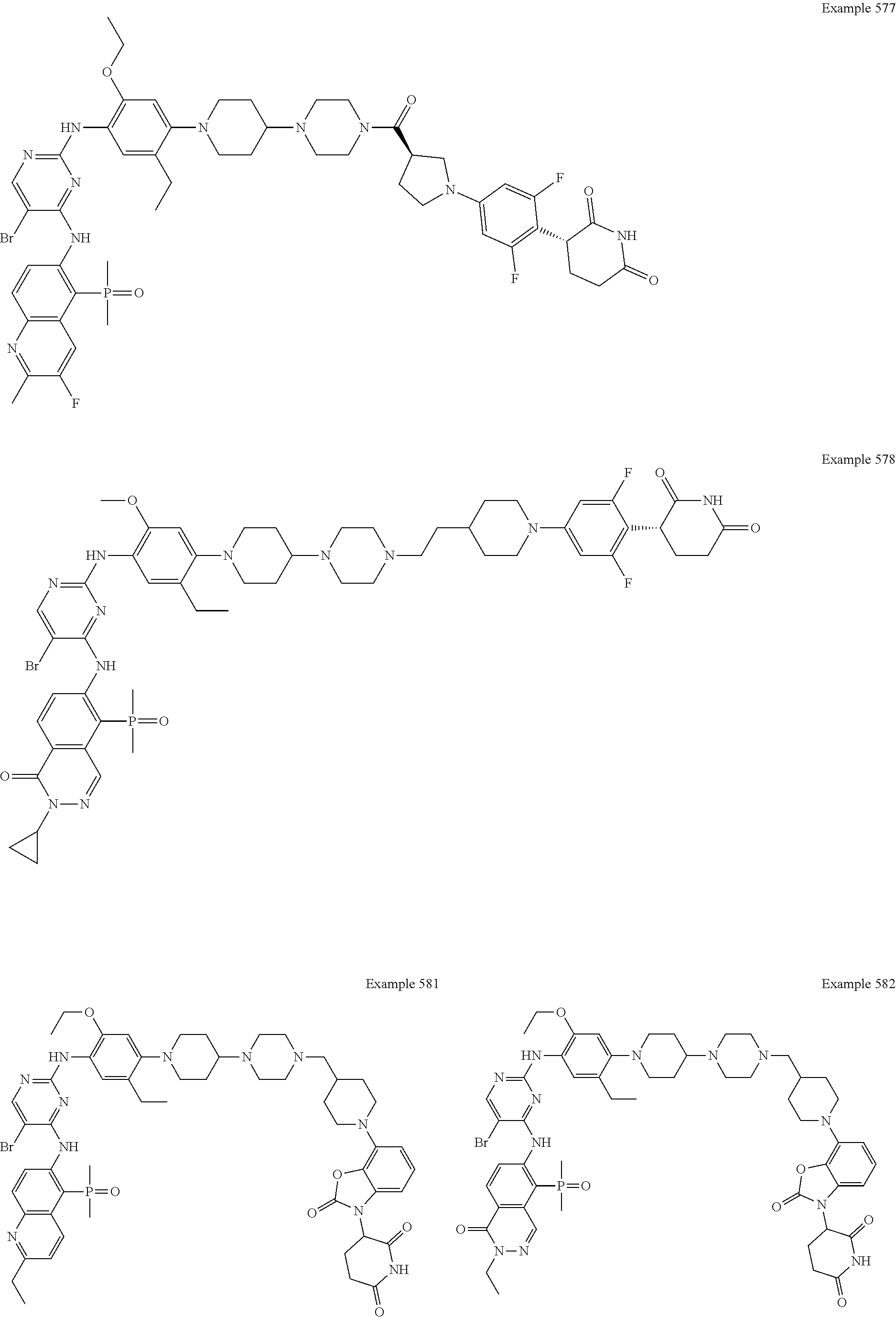

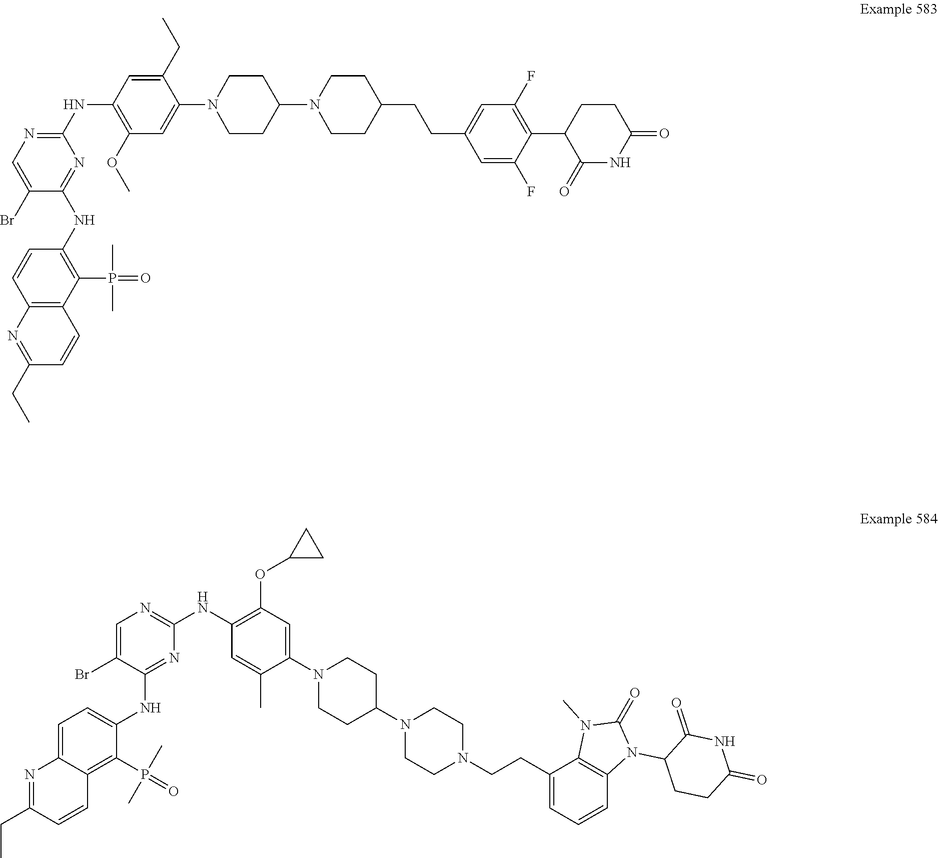 [SMILES: CCc1ccc2c(P(C)(C)=O)c(Nc3nc(Nc4cc(CC)c(N5CCC(N6CCC(CCc7cc(F)c(C8CCC(=O)NC8=O)c(F)c7)CC6)CC5)cc4OC)ncc3Br)ccc2n1 ; CCc1ccc2c(P(C)(C)=O)c(Nc3nc(Nc4cc(C)c(N5CCC(N6CCN(CCc7cccc8c7n(C)c(=O)n8C7CCC(=O)NC7=O)CC6)CC5)cc4OC4CC4)ncc3Br)ccc2n1]

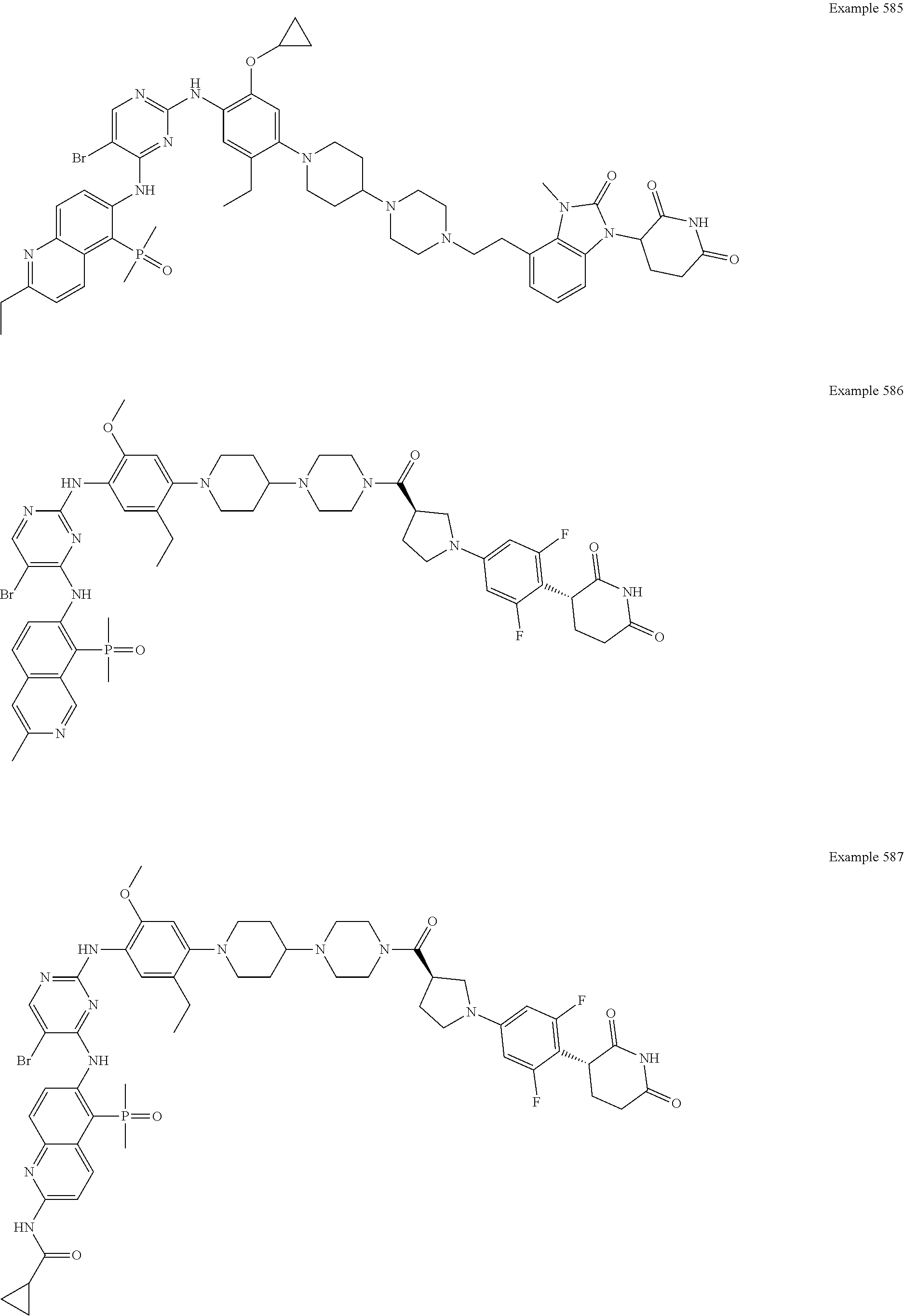 [SMILES: CCc1ccc2c(P(C)(C)=O)c(Nc3nc(Nc4cc(CC)c(N5CCC(N6CCN(CCc7cccc8c7n(C)c(=O)n8C7CCC(=O)NC7=O)CC6)CC5)cc4OC4CC4)ncc3Br)ccc2n1 ; CCc1cc(Nc2ncc(Br)c(Nc3ccc4cc(C)ncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; CCc1cc(Nc2ncc(Br)c(Nc3ccc4nc(NC(=O)C5CC5)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

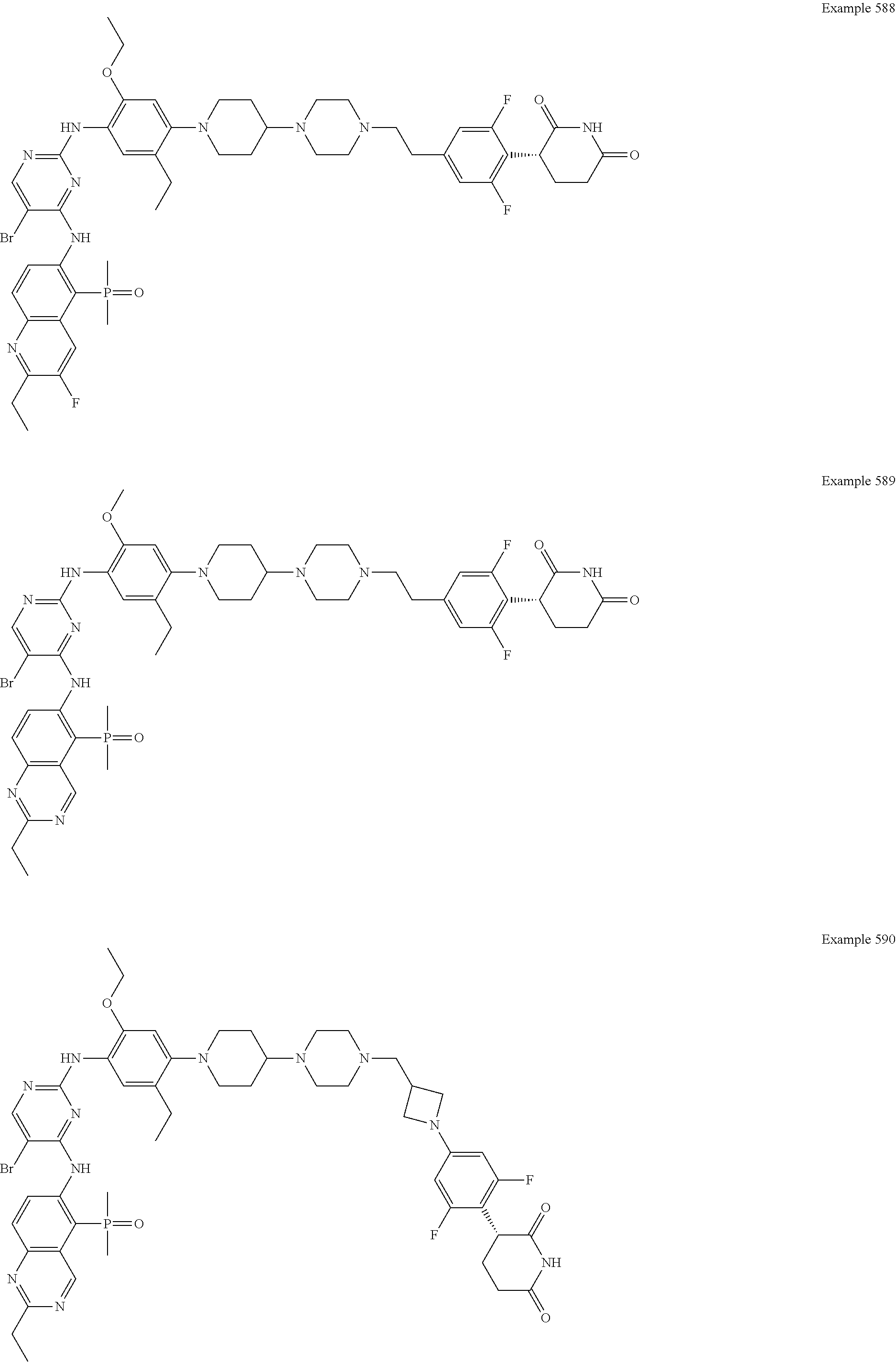 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)c(F)cc3c2P(C)(C)=O)n1 ; CCc1ncc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC)ncc3Br)ccc2n1 ; CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3nc(CC)ncc3c2P(C)(C)=O)n1]

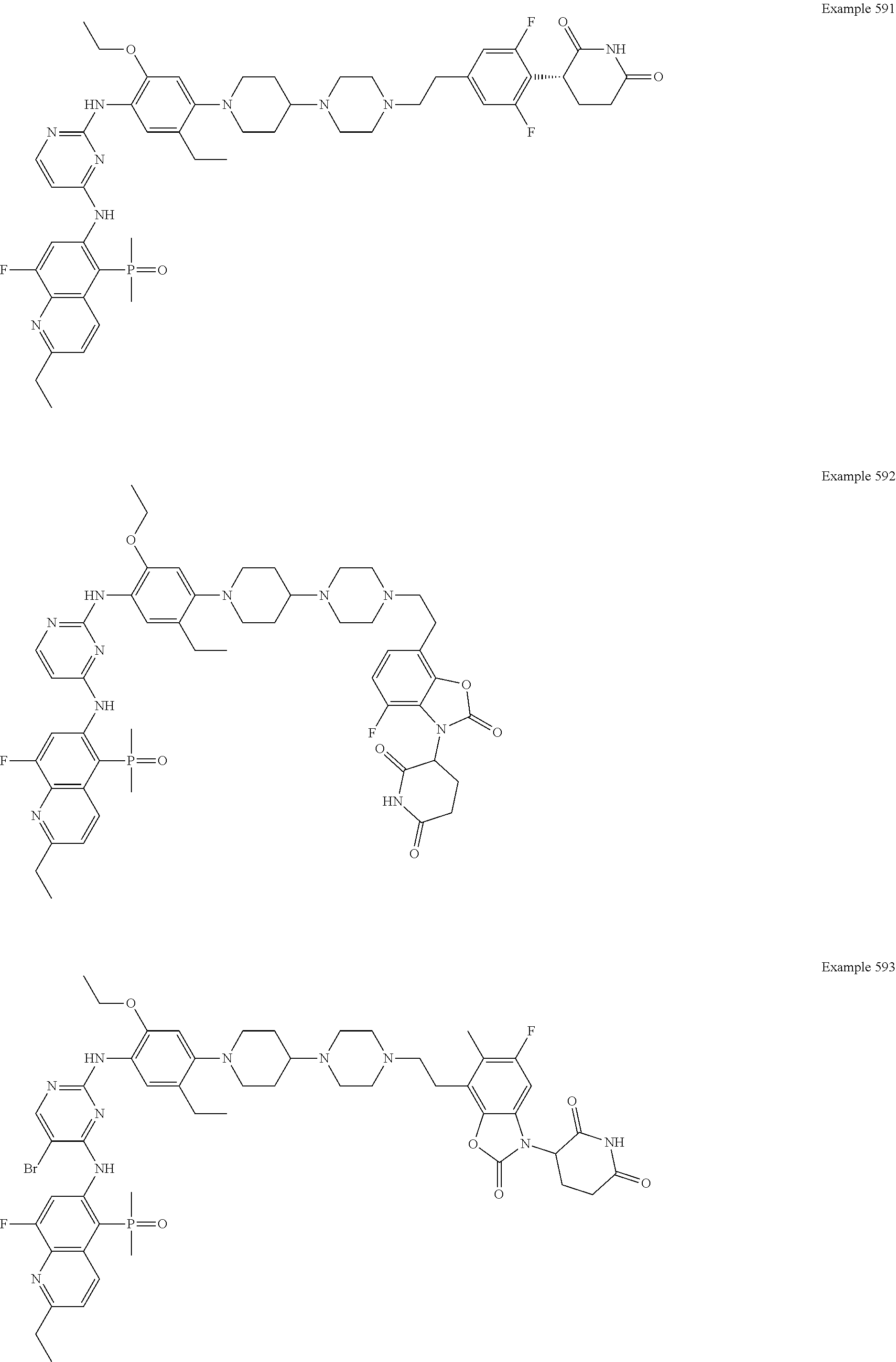 [SMILES: CCOc1cc(N2CCC(N3CCN(CCc4cc(F)c([C@H]5CCC(=O)NC5=O)c(F)c4)CC3)CC2)c(CC)cc1Nc1nccc(Nc2cc(F)c3nc(CC)ccc3c2P(C)(C)=O)n1 ; CCOc1cc(N2CCC(N3CCN(CCc4ccc(F)c5c4oc(=O)n5C4CCC(=O)NC4=O)CC3)CC2)c(CC)cc1Nc1nccc(Nc2cc(F)c3nc(CC)ccc3c2P(C)(C)=O)n1 ; CCOc1cc(N2CCC(N3CCN(CCc4c(C)c(F)cc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1]

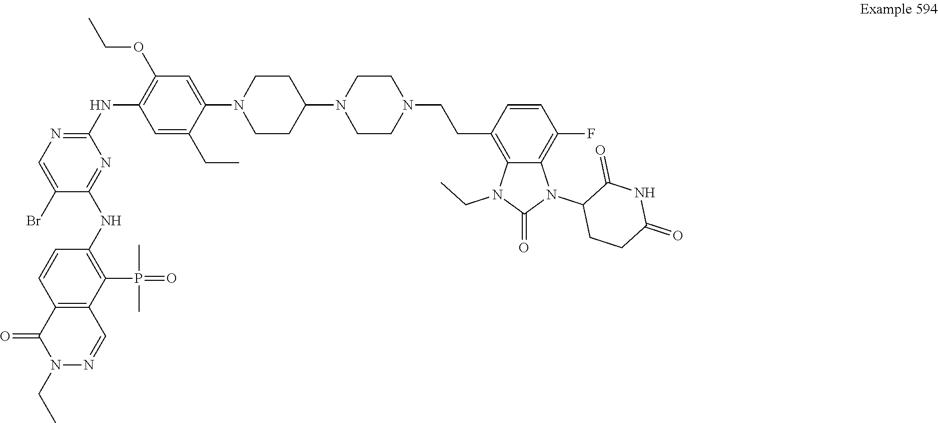 [SMILES: CCOc1cc(N2CCC(N3CCN(CCc4ccc(F)c5c4n(CC)c(=O)n5C4CCC(=O)NC4=O)CC3)CC2)c(CC)cc1Nc1ncc(Br)c(Nc2ccc3c(=O)n(CC)ncc3c2P(C)(C)=O)n1]

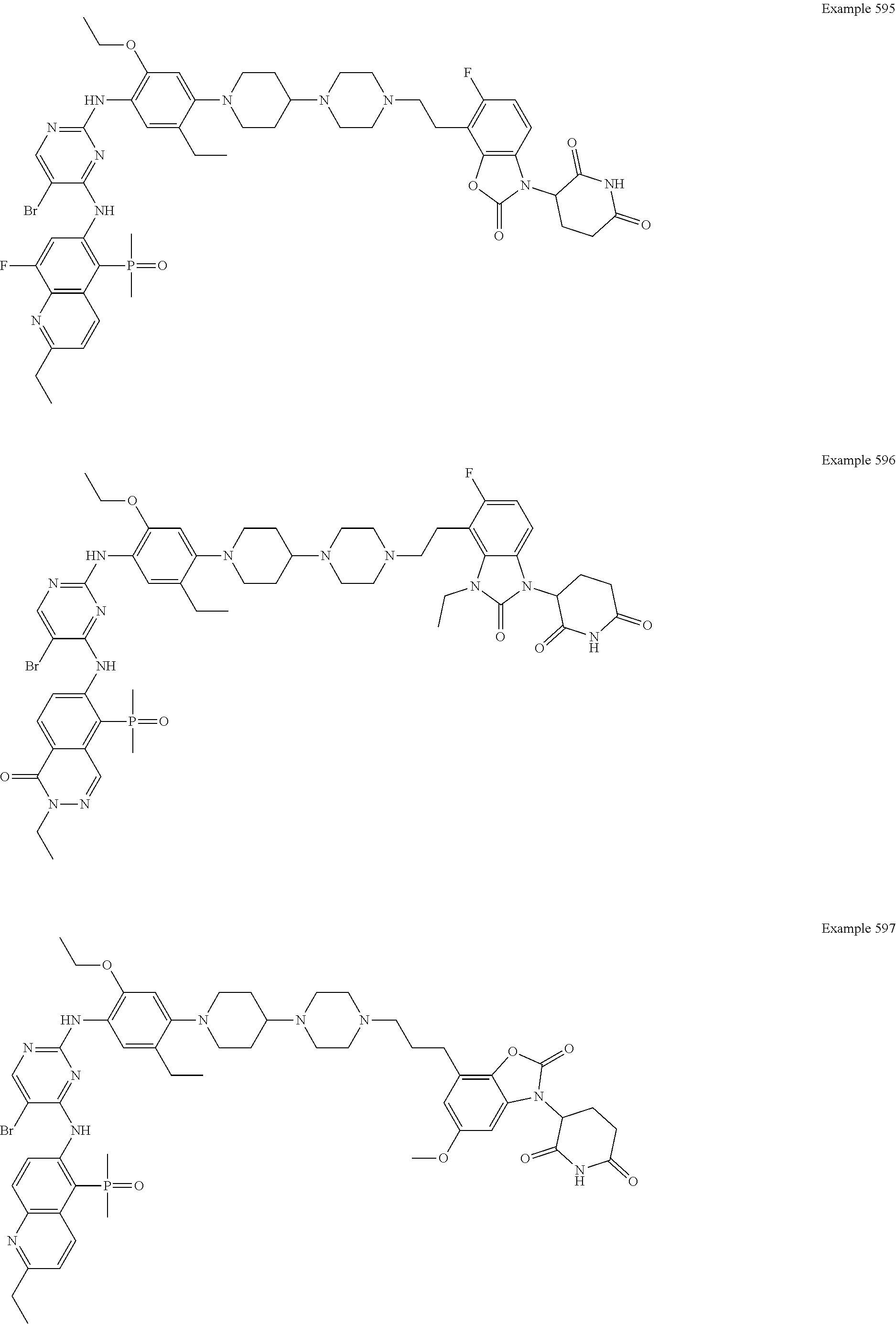 [SMILES: CCOc1cc(N2CCC(N3CCN(CCc4c(F)ccc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1 ; CCOc1cc(N2CCC(N3CCN(CCc4c(F)ccc5c4n(CC)c(=O)n5C4CCC(=O)NC4=O)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCCc4cc(OC)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

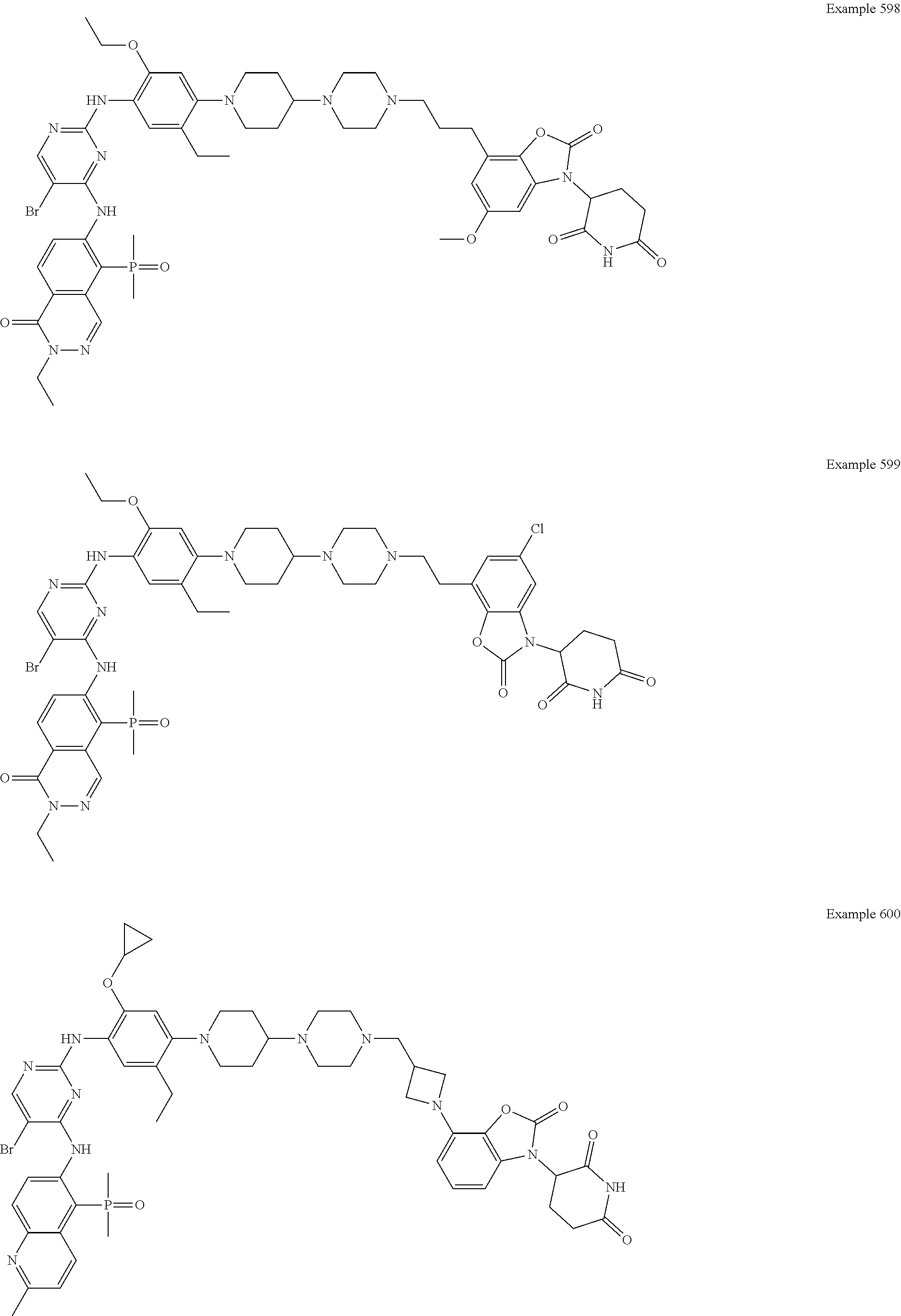 [SMILES: CCOc1cc(N2CCC(N3CCN(CCCc4cc(OC)cc5c4oc(=O)n5C4CCC(=O)NC4=O)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCc4cc(Cl)cc5c4oc(=O)n5C4CCC(=O)NC4=O)CC3)CC2)c(CC)cc1Nc1ncc(Br)c(Nc2ccc3c(=O)n(CC)ncc3c2P(C)(C)=O)n1 ; CCc1cc(Nc2ncc(Br)c(Nc3ccc4nc(C)ccc4c3P(C)(C)=O)n2)c(OC2CC2)cc1N1CCC(N2CCN(CC3CN(c4cccc5c4oc(=O)n5C4CCC(=O)NC4=O)C3)CC2)CC1]

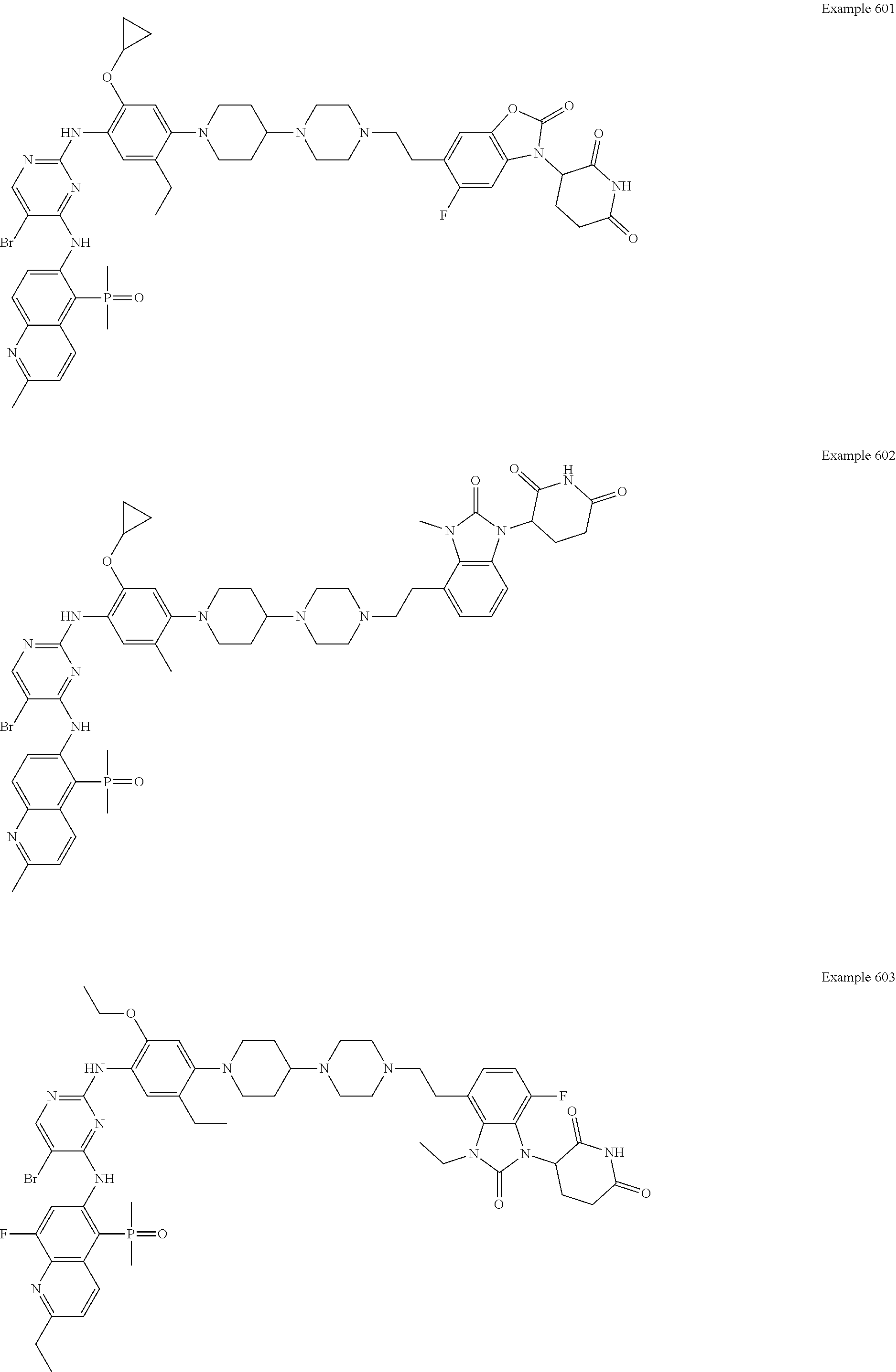 [SMILES: CCc1cc(Nc2ncc(Br)c(Nc3ccc4nc(C)ccc4c3P(C)(C)=O)n2)c(OC2CC2)cc1N1CCC(N2CCN(CCc3cc4oc(=O)n(C5CCC(=O)NC5=O)c4cc3F)CC2)CC1 ; Cc1ccc2c(P(C)(C)=O)c(Nc3nc(Nc4cc(C)c(N5CCC(N6CCN(CCc7cccc8c7n(C)c(=O)n8C7CCC(=O)NC7=O)CC6)CC5)cc4OC4CC4)ncc3Br)ccc2n1 ; CCOc1cc(N2CCC(N3CCN(CCc4ccc(F)c5c4n(CC)c(=O)n5C4CCC(=O)NC4=O)CC3)CC2)c(CC)cc1Nc1ncc(Br)c(Nc2cc(F)c3nc(CC)ccc3c2P(C)(C)=O)n1]

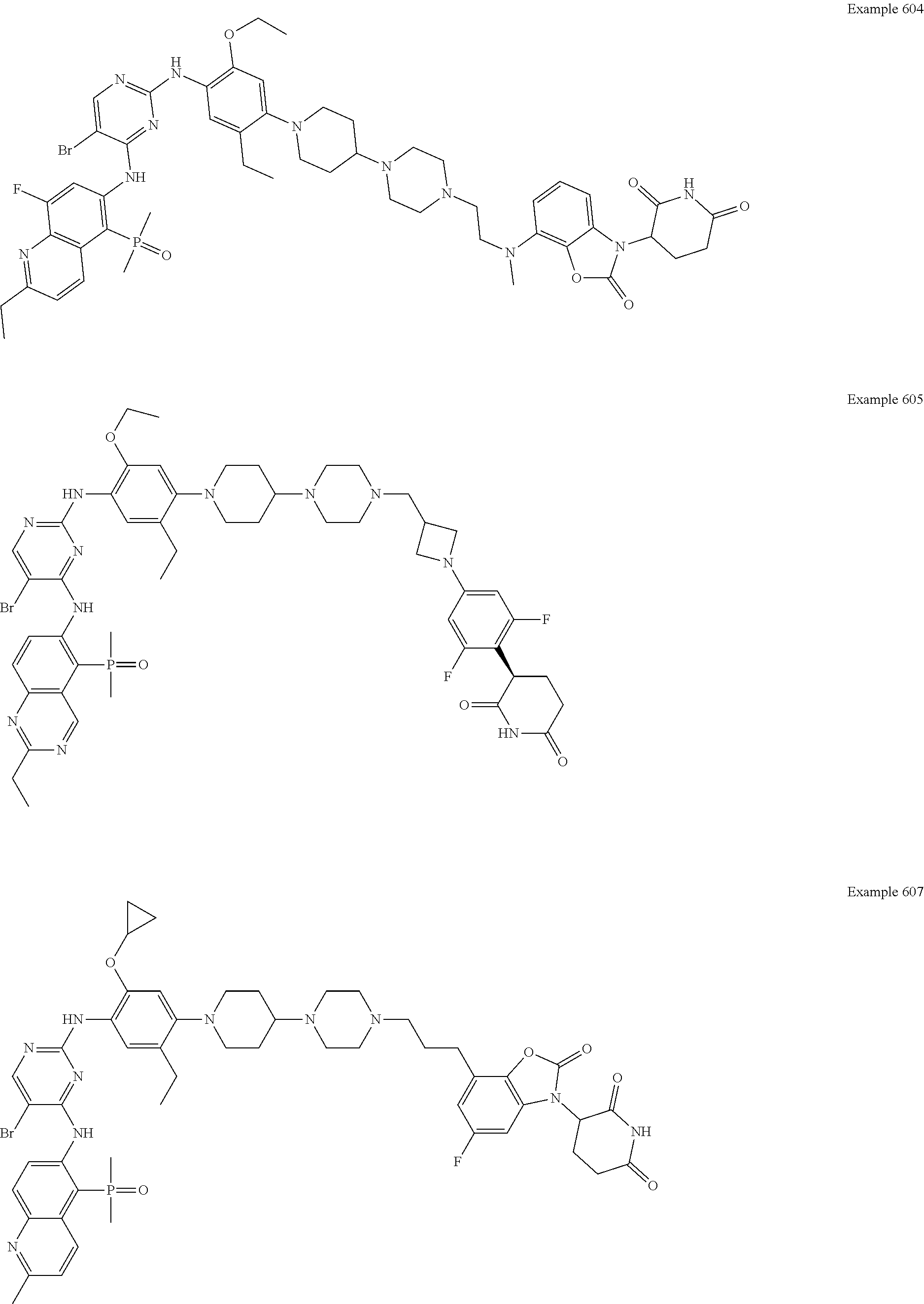 [SMILES: CCOc1cc(N2CCC(N3CCN(CCN(C)c4cccc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1 ; CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3nc(CC)ncc3c2P(C)(C)=O)n1 ; CCc1cc(Nc2ncc(Br)c(Nc3ccc4nc(C)ccc4c3P(C)(C)=O)n2)c(OC2CC2)cc1N1CCC(N2CCN(CCCc3cc(F)cc4c3oc(=O)n4C3CCC(=O)NC3=O)CC2)CC1]

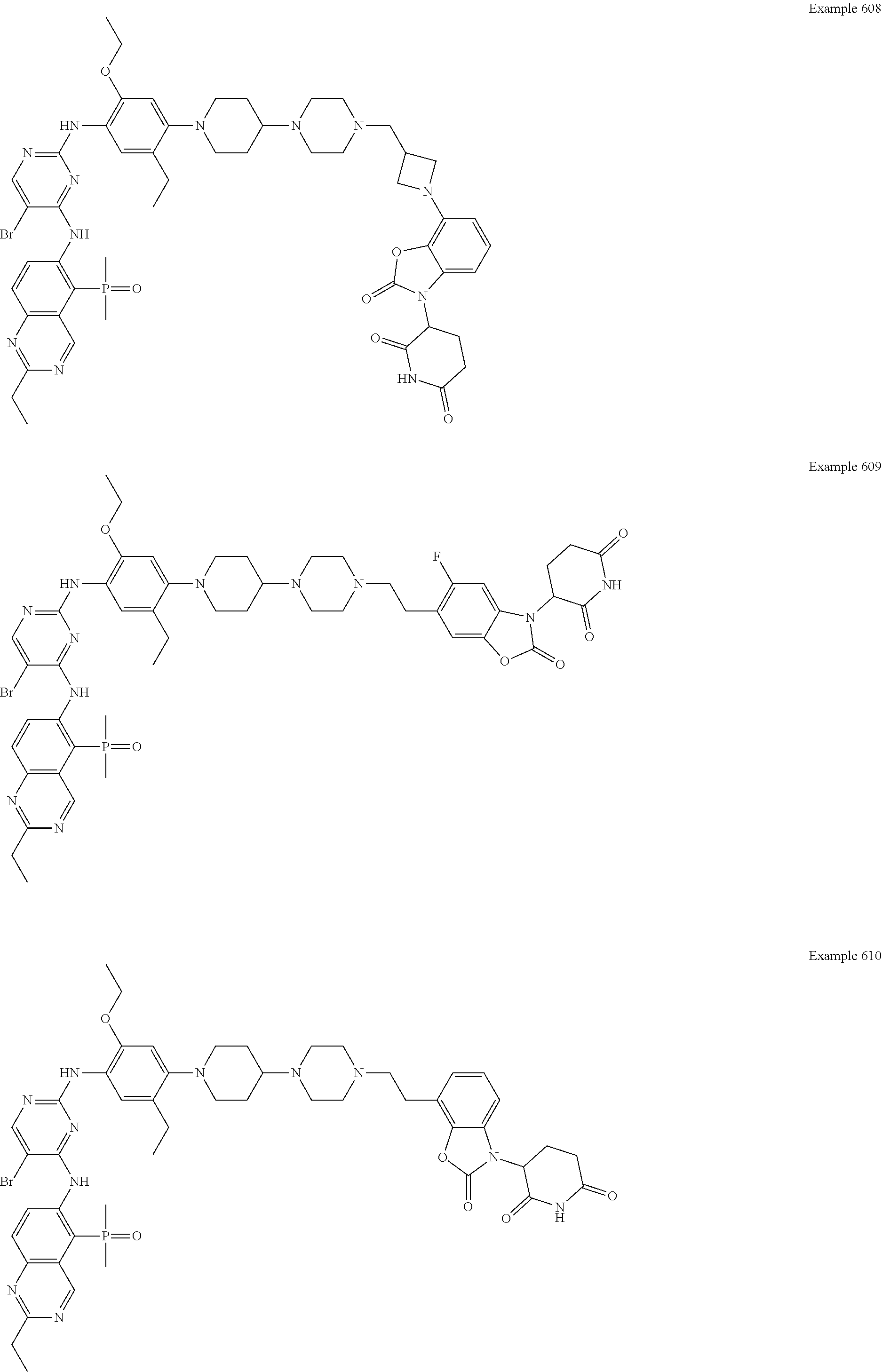 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3nc(CC)ncc3c2P(C)(C)=O)n1 ; CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2ccc3nc(CC)ncc3c2P(C)(C)=O)n1 ; CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3nc(CC)ncc3c2P(C)(C)=O)n1]

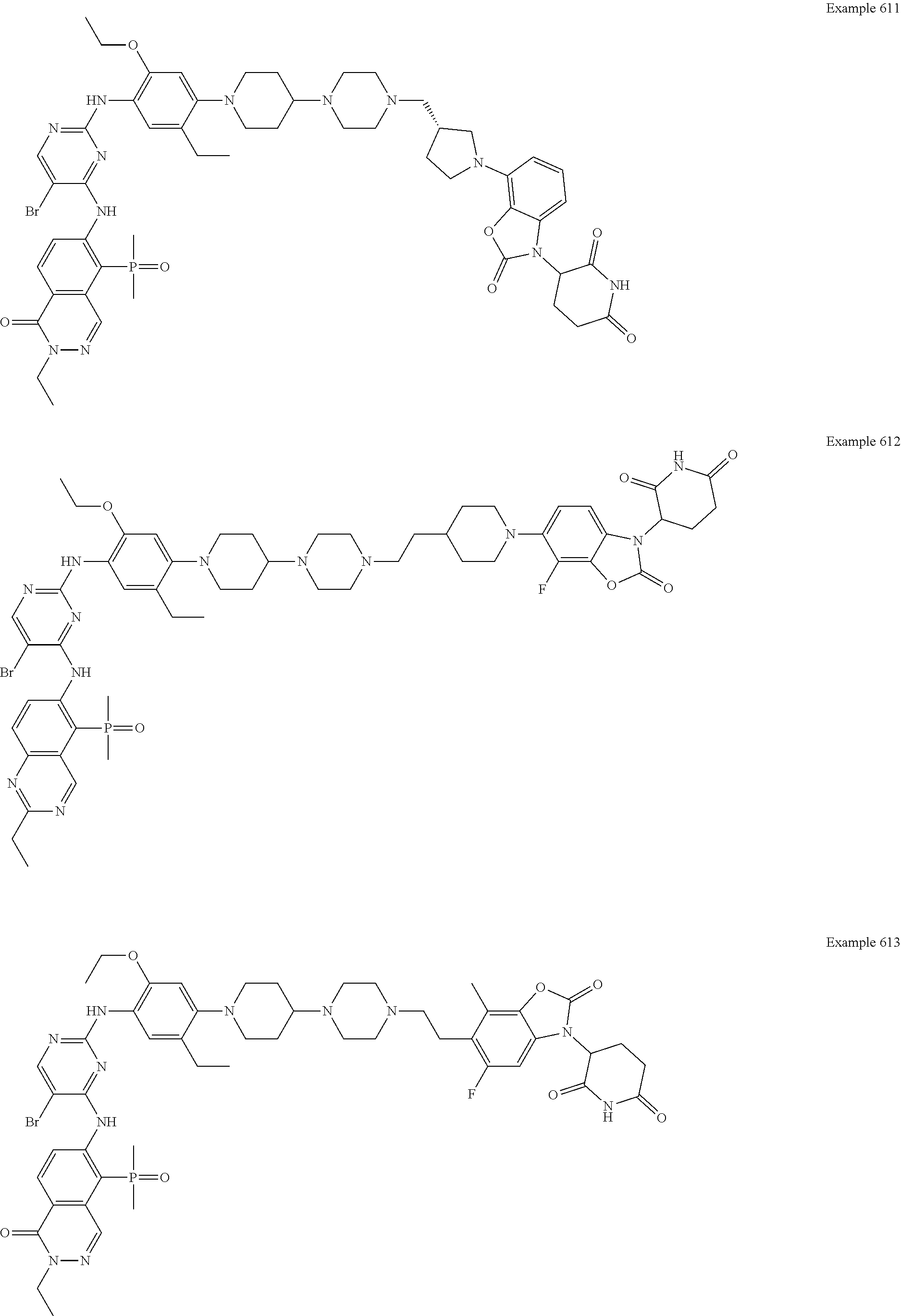 [SMILES: CCOc1cc(N2CCC(N3CCN(C[C@H]4CCN(c5cccc6c5oc(=O)n6C5CCC(=O)NC5=O)C4)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCC4CCN(c5ccc6c(oc(=O)n6C6CCC(=O)NC6=O)c5F)CC4)CC3)CC2)c(CC)cc1Nc1ncc(Br)c(Nc2ccc3nc(CC)ncc3c2P(C)(C)=O)n1 ; CCOc1cc(N2CCC(N3CCN(CCc4c(F)cc5c(oc(=O)n5C5CCC(=O)NC5=O)c4C)CC3)CC2)c(CC)cc1Nc1ncc(Br)c(Nc2ccc3c(=O)n(CC)ncc3c2P(C)(C)=O)n1]

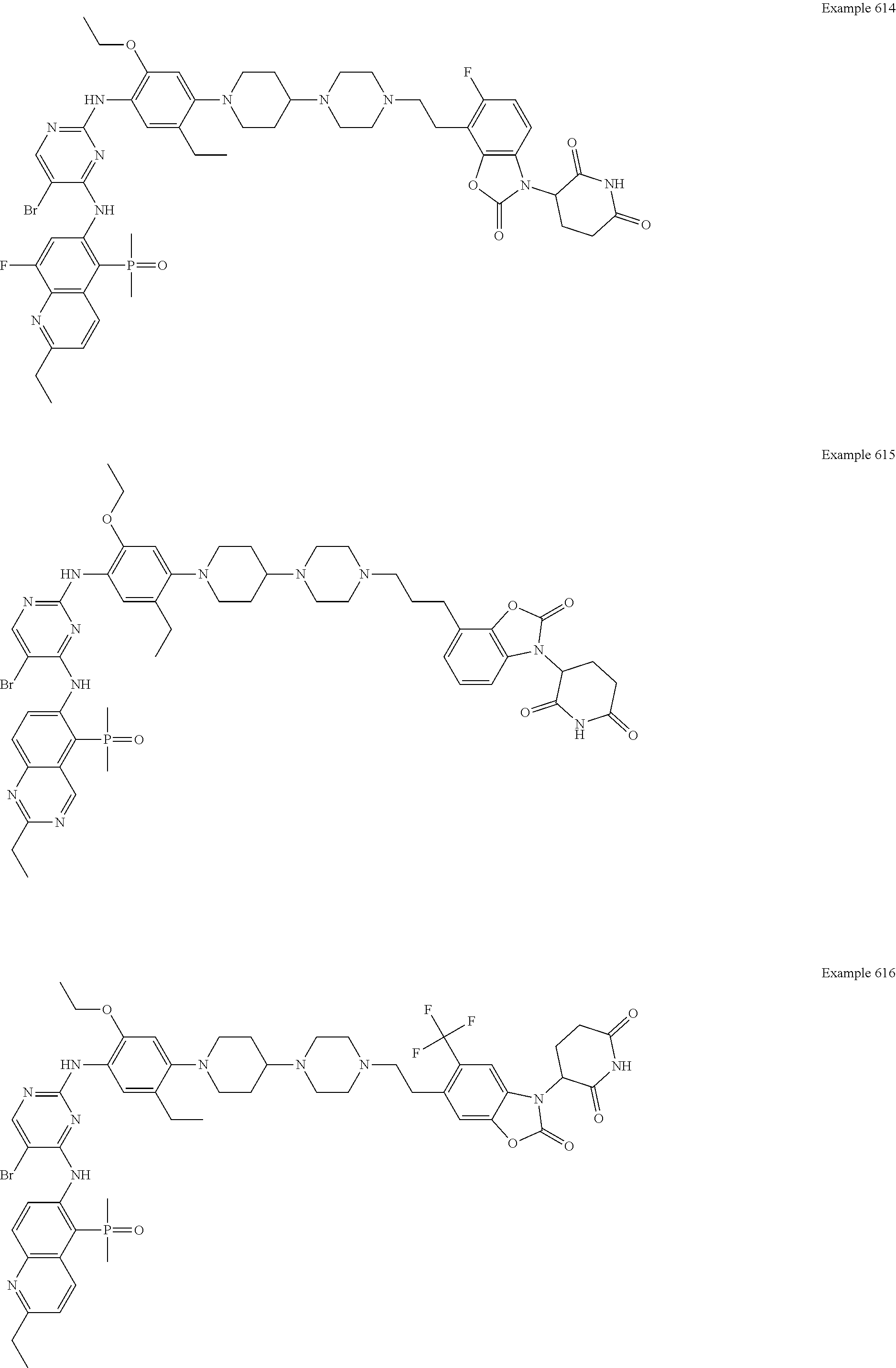 [SMILES: CCOc1cc(N2CCC(N3CCN(CCc4c(F)ccc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1 ; CCOc1cc(N2CCC(N3CCN(CCCc4cccc5c4oc(=O)n5C4CCC(=O)NC4=O)CC3)CC2)c(CC)cc1Nc1ncc(Br)c(Nc2ccc3nc(CC)ncc3c2P(C)(C)=O)n1 ; CCOc1cc(N2CCC(N3CCN(CCc4cc5oc(=O)n(C6CCC(=O)NC6=O)c5cc4C(F)(F)F)CC3)CC2)c(CC)cc1Nc1ncc(Br)c(Nc2ccc3nc(CC)ccc3c2P(C)(C)=O)n1]

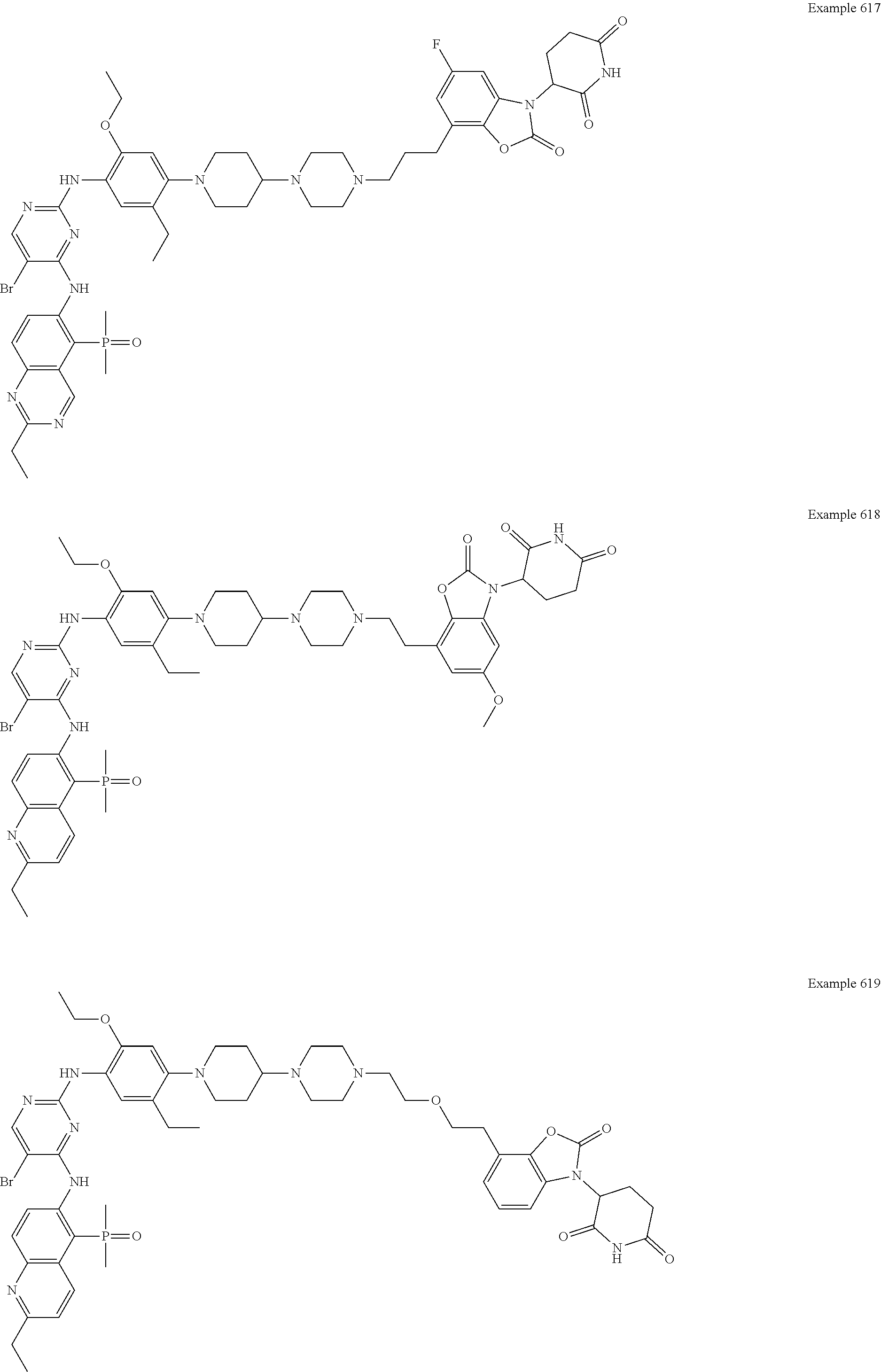 [SMILES: CCOc1cc(N2CCC(N3CCN(CCCc4cc(F)cc5c4oc(=O)n5C4CCC(=O)NC4=O)CC3)CC2)c(CC)cc1Nc1ncc(Br)c(Nc2ccc3nc(CC)ncc3c2P(C)(C)=O)n1 ; CCOc1cc(N2CCC(N3CCN(CCc4cc(OC)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCOCCc4cccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

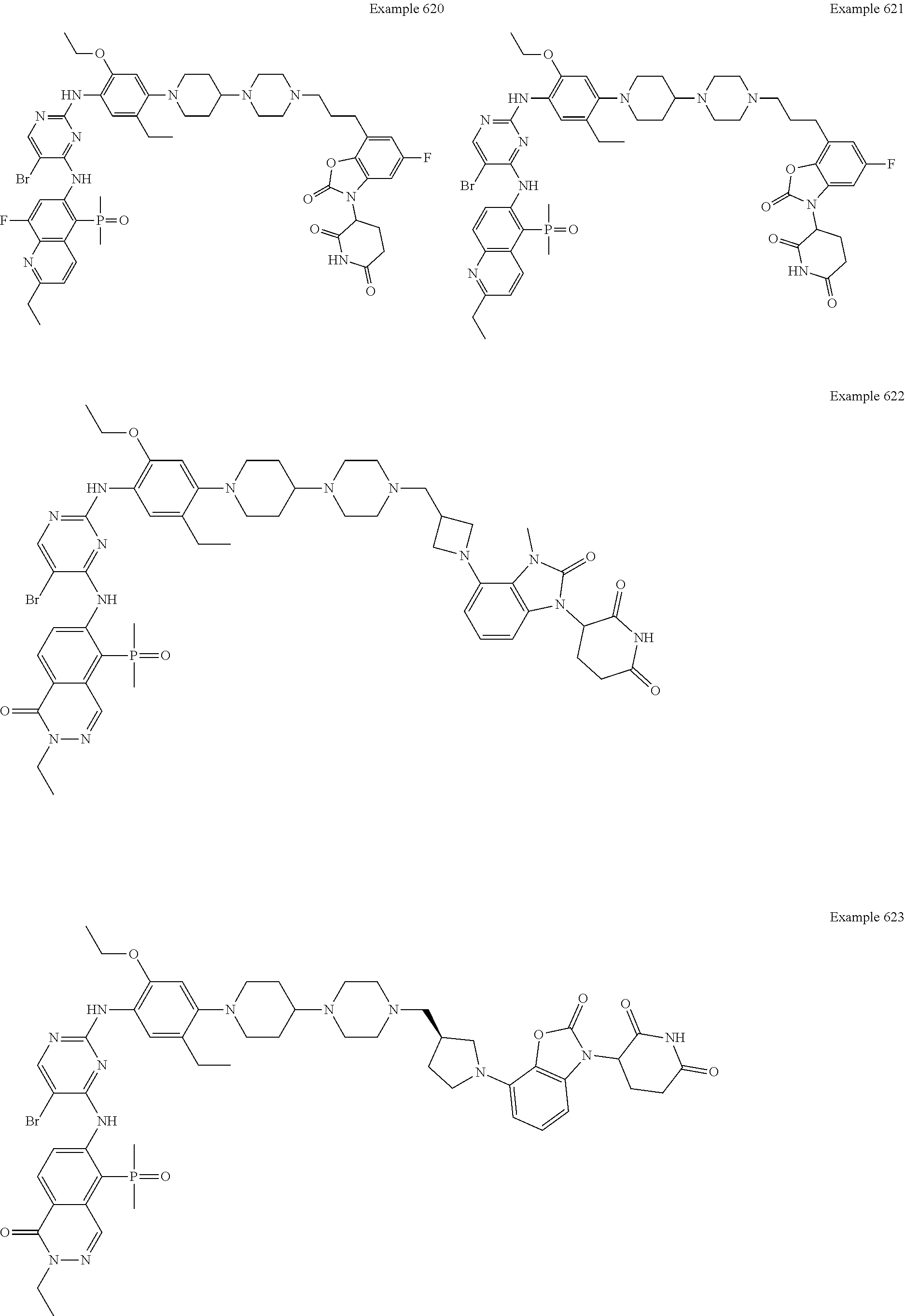

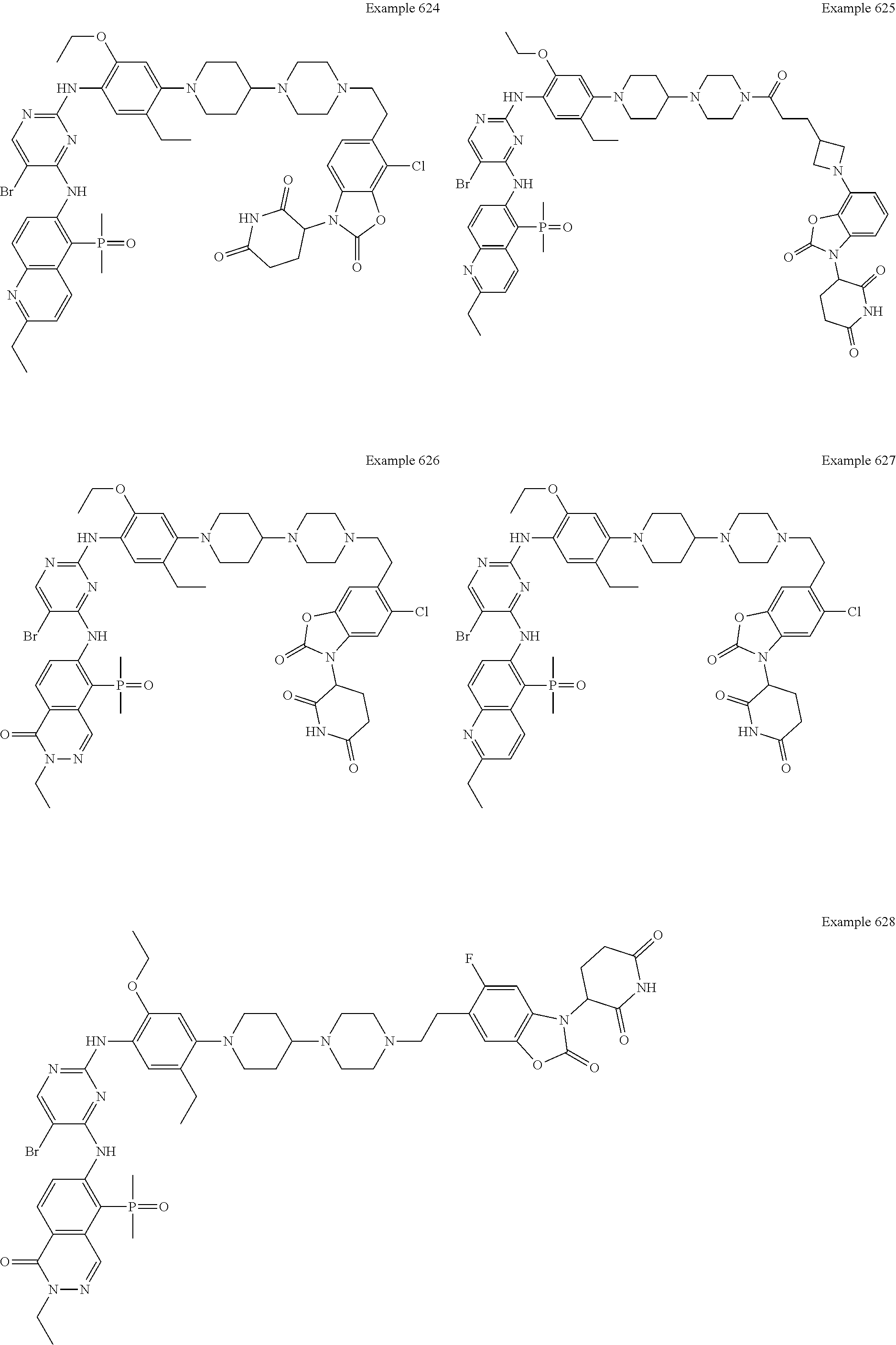

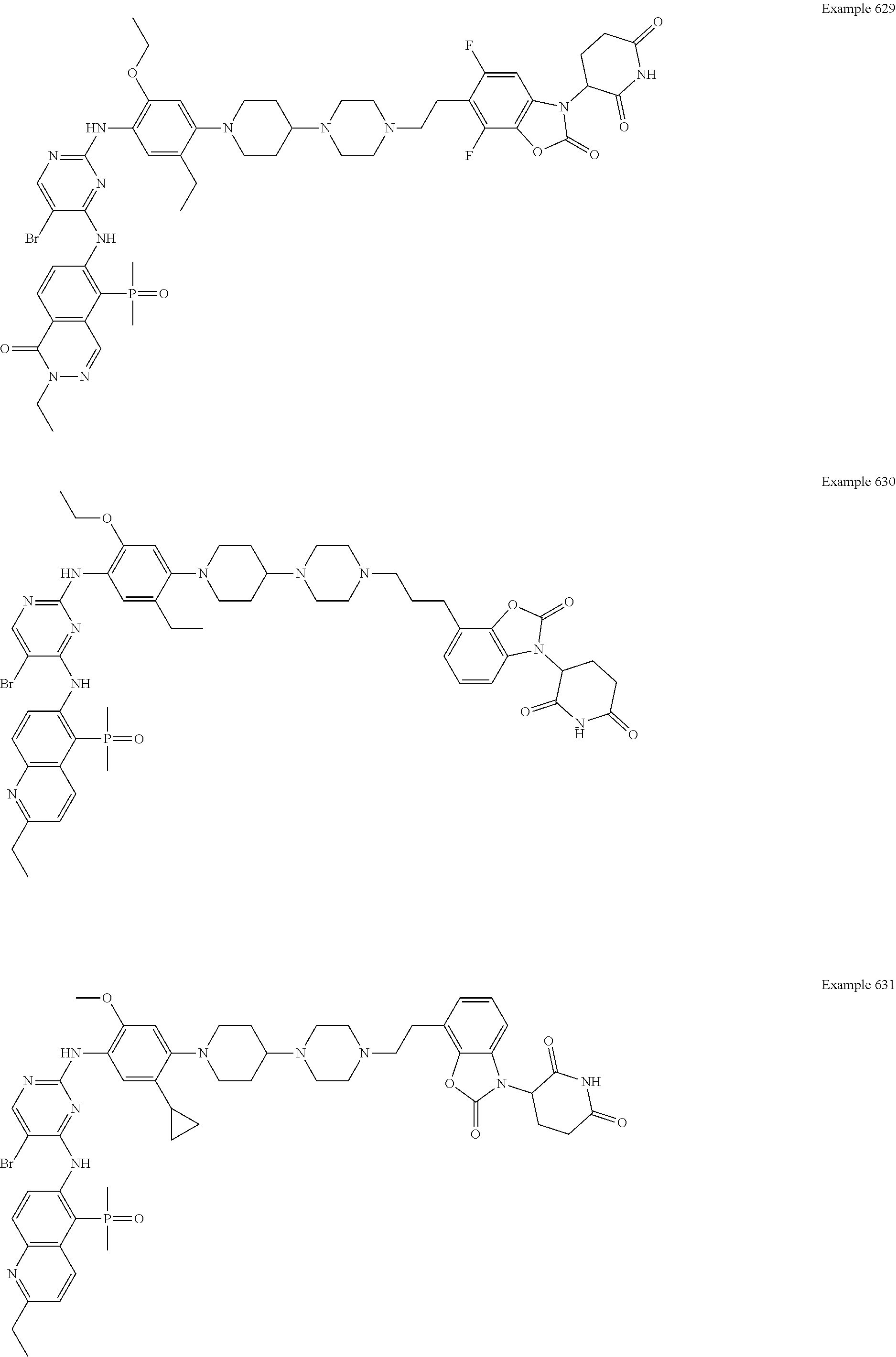 [SMILES: CCOc1cc(N2CCC(N3CCN(CCc4c(F)cc5c(oc(=O)n5C5CCC(=O)NC5=O)c4F)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCCc4cccc5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCc1ccc2c(P(C)(C)=O)c(Nc3nc(Nc4cc(C5CC5)c(N5CCC(N6CCN(CCc7cccc8c7oc(=O)n8C7CCC(=O)NC7=O)CC6)CC5)cc4OC)ncc3Br)ccc2n1]

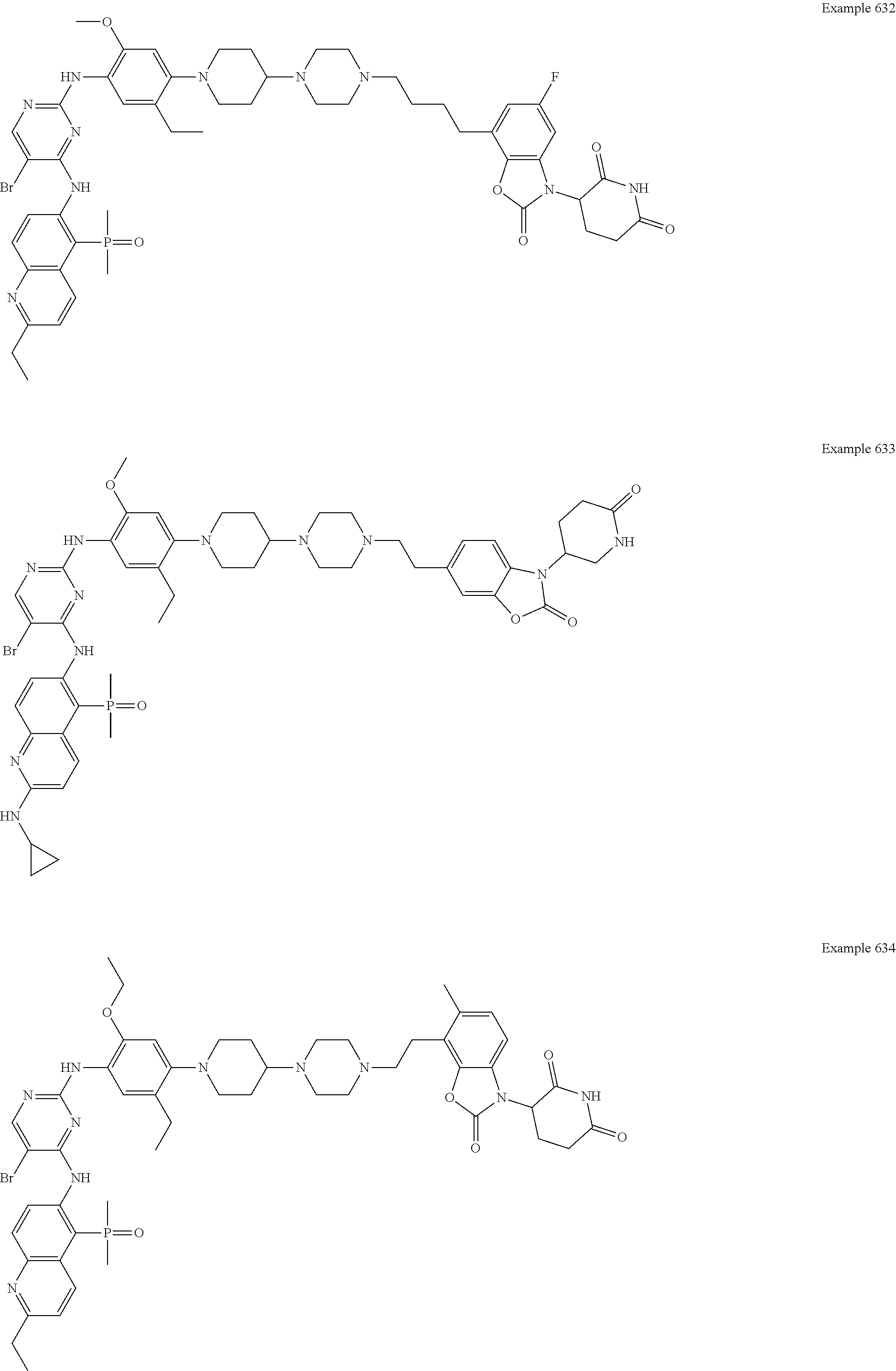 [SMILES: CCc1ccc2c(P(C)(C)=O)c(Nc3nc(Nc4cc(CC)c(N5CCC(N6CCN(CCCCc7cc(F)cc8c7oc(=O)n8C7CCC(=O)NC7=O)CC6)CC5)cc4OC)ncc3Br)ccc2n1 ; CCc1cc(Nc2ncc(Br)c(Nc3ccc4nc(NC5CC5)ccc4c3P(C)(C)=O)n2)c(OC)cc1N1CCC(N2CCN(CCc3ccc4c(c3)oc(=O)n4C3CCC(=O)NC3)CC2)CC1 ; CCOc1cc(N2CCC(N3CCN(CCc4c(C)ccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

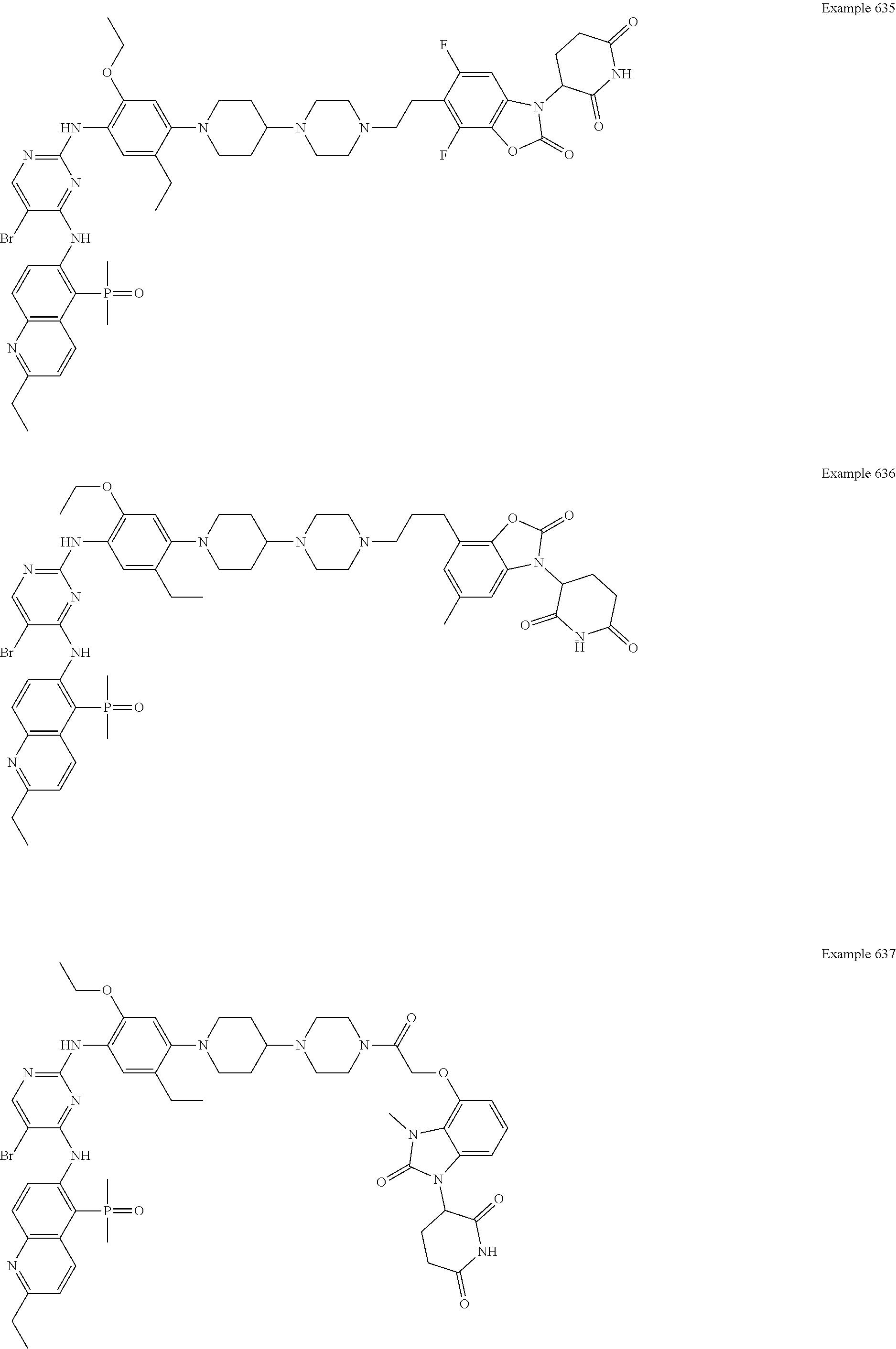 [SMILES: CCOc1cc(N2CCC(N3CCN(CCc4c(F)cc5c(oc(=O)n5C5CCC(=O)NC5=O)c4F)CC3)CC2)c(CC)cc1Nc1ncc(Br)c(Nc2ccc3nc(CC)ccc3c2P(C)(C)=O)n1 ; CCOc1cc(N2CCC(N3CCN(CCCc4cc(C)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(C(=O)COc4cccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

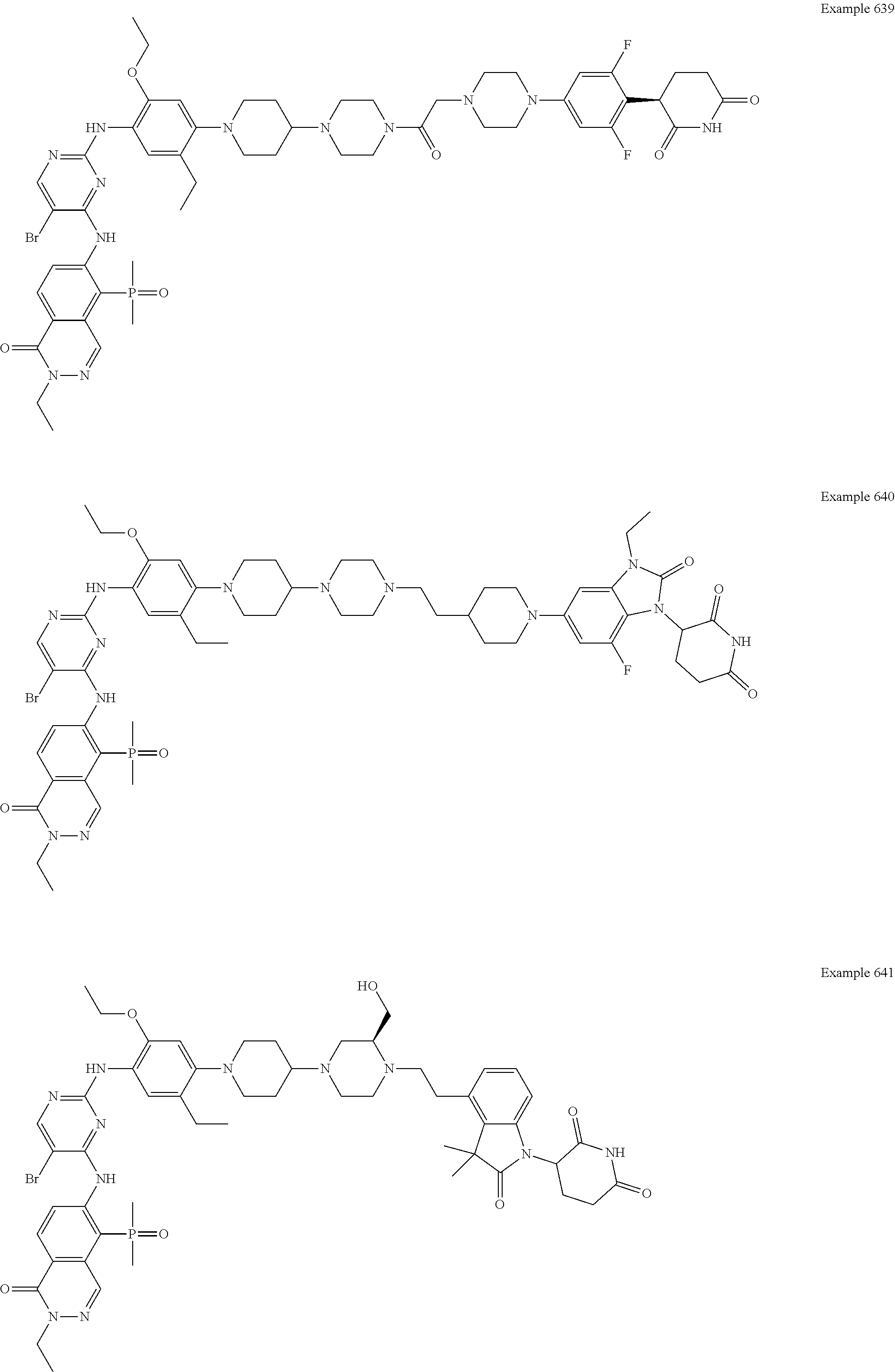 [SMILES: CCOc1cc(N2CCC(N3CCN(C(=O)CN4CCN(c5cc(F)c([C@H]6CCC(=O)NC6=O)c(F)c5)CC4)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCC4CCN(c5cc(F)c6c(c5)n(CC)c(=O)n6C5CCC(=O)NC5=O)CC4)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCc4cccc5c4C(C)(C)C(=O)N5C4CCC(=O)NC4=O)[C@H](CO)C3)CC2)c(CC)cc1Nc1ncc(Br)c(Nc2ccc3c(=O)n(CC)ncc3c2P(C)(C)=O)n1]

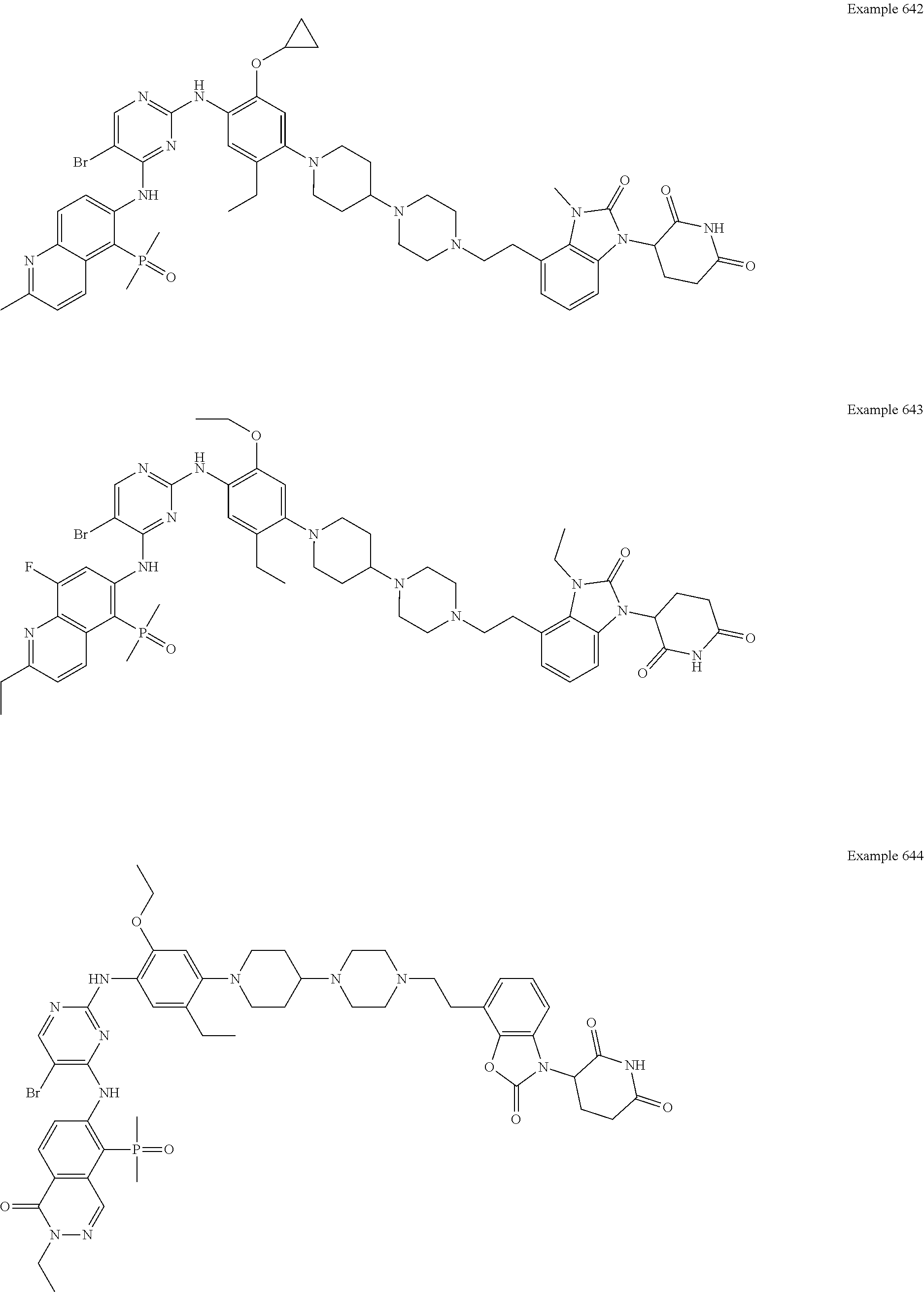 [SMILES: CCc1cc(Nc2ncc(Br)c(Nc3ccc4nc(C)ccc4c3P(C)(C)=O)n2)c(OC2CC2)cc1N1CCC(N2CCN(CCc3cccc4c3n(C)c(=O)n4C3CCC(=O)NC3=O)CC2)CC1 ; CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2cc(F)c3nc(CC)ccc3c2P(C)(C)=O)n1 ; CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3c(=O)n(CC)ncc3c2P(C)(C)=O)n1]

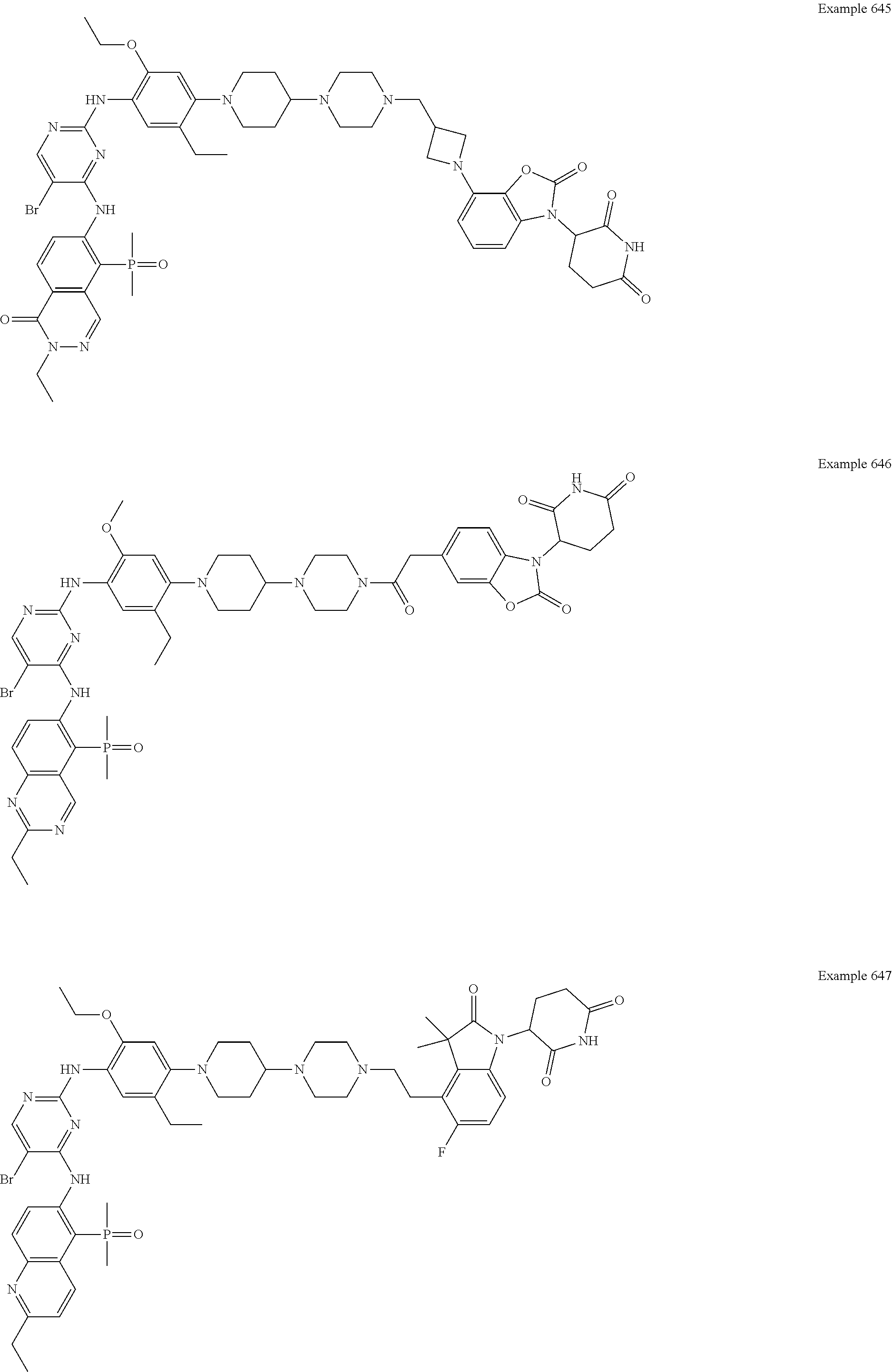 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3c(=O)n(CC)ncc3c2P(C)(C)=O)n1 ; CCc1ncc2c(P(C)(C)=O)c(Nc3nc(Nc4cc(CC)c(N5CCC(N6CCN(C(=O)Cc7ccc8c(c7)oc(=O)n8C7CCC(=O)NC7=O)CC6)CC5)cc4OC)ncc3Br)ccc2n1 ; CCOc1cc(N2CCC(N3CCN(CCc4c(F)ccc5c4C(C)(C)C(=O)N5C4CCC(=O)NC4=O)CC3)CC2)c(CC)cc1Nc1ncc(Br)c(Nc2ccc3nc(CC)ccc3c2P(C)(C)=O)n1]

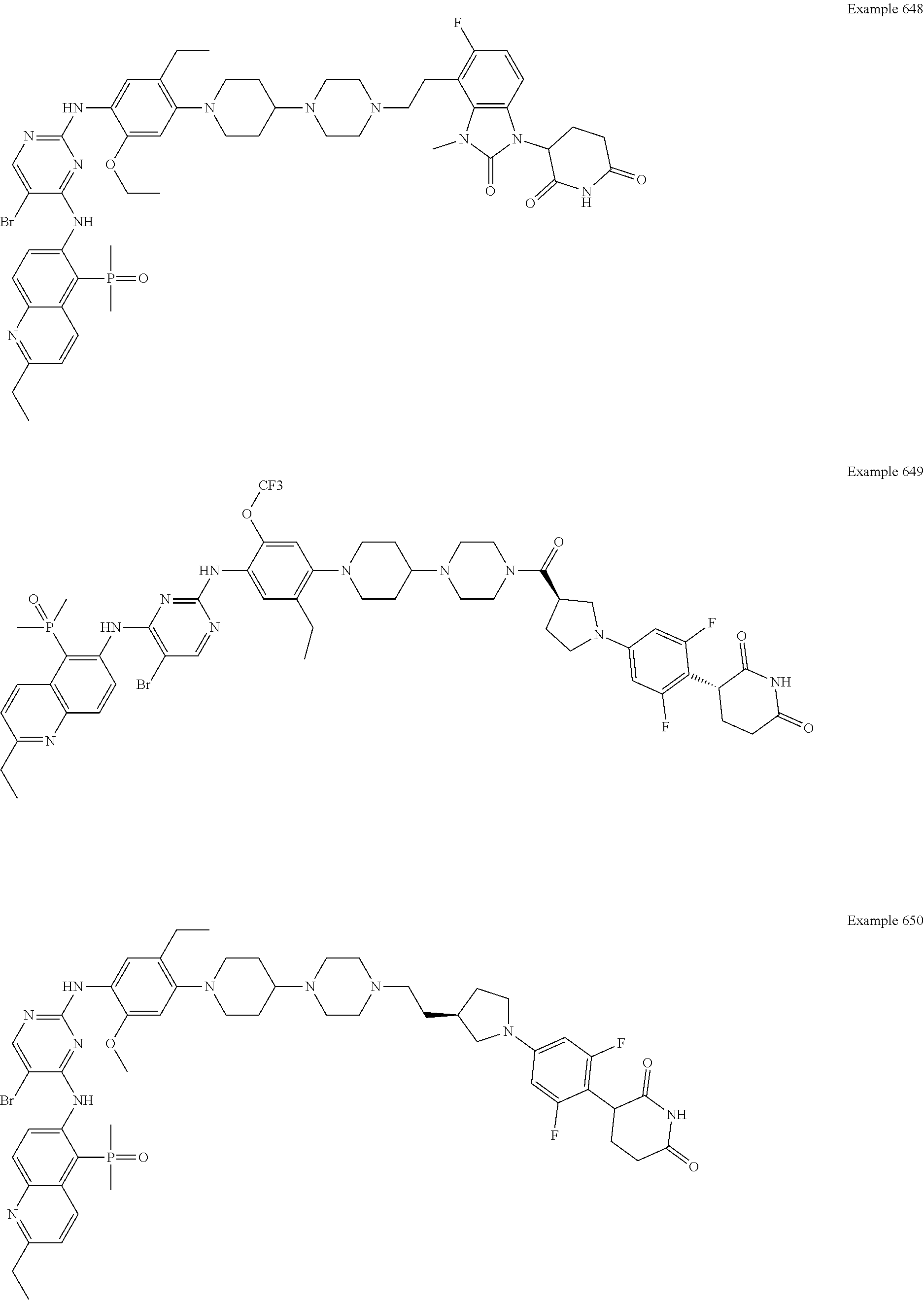 [SMILES: CCOc1cc(N2CCC(N3CCN(CCc4c(F)ccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1 ; CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC(F)(F)F)ncc3Br)ccc2n1 ; CCc1ccc2c(P(C)(C)=O)c(Nc3nc(Nc4cc(CC)c(N5CCC(N6CCN(CC[C@H]7CCN(c8cc(F)c(C9CCC(=O)NC9=O)c(F)c8)C7)CC6)CC5)cc4OC)ncc3Br)ccc2n1]

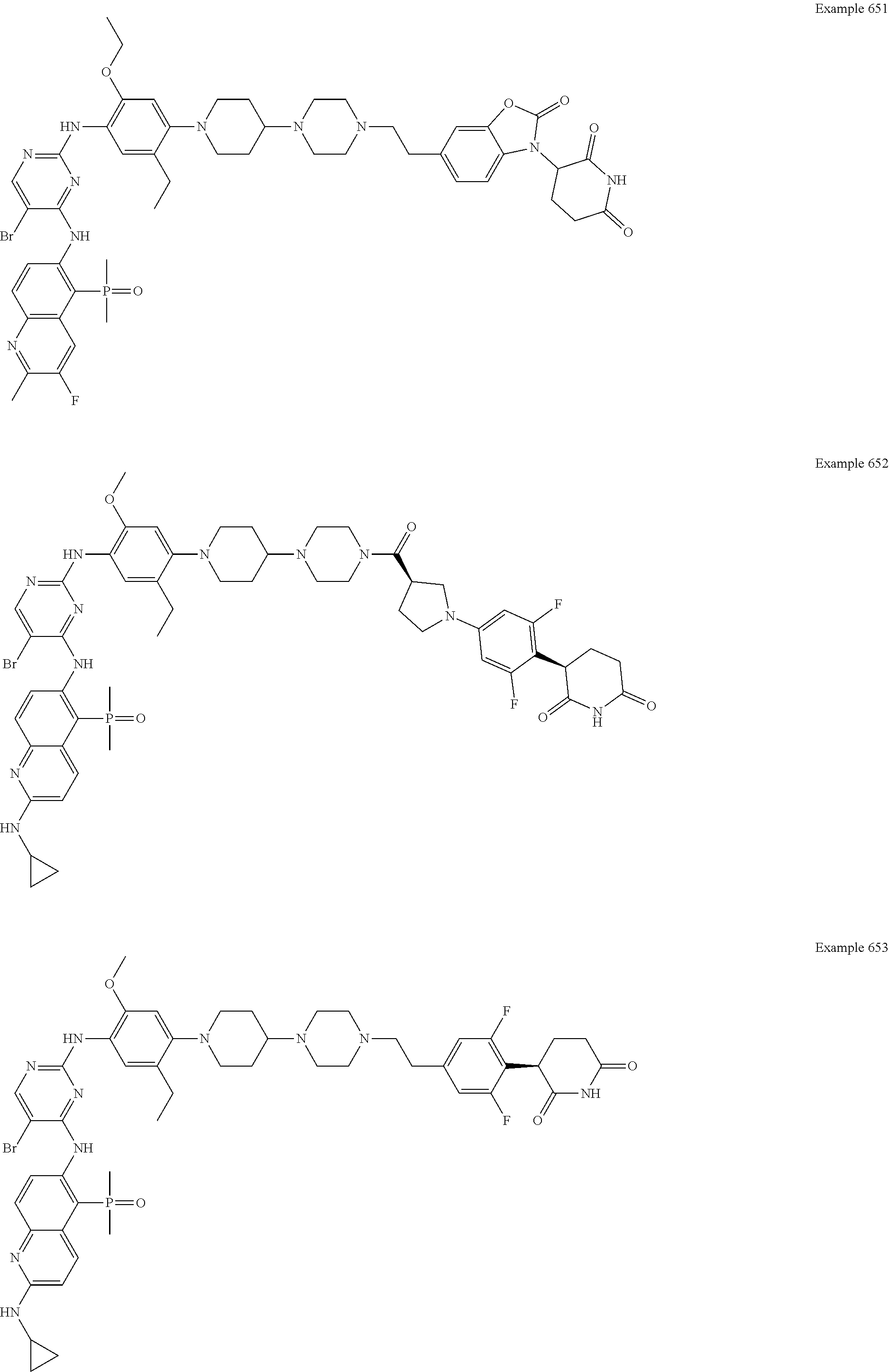 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2ccc3nc(C)c(F)cc3c2P(C)(C)=O)n1 ; CCc1cc(Nc2ncc(Br)c(Nc3ccc4nc(NC5CC5)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; CCc1cc(Nc2ncc(Br)c(Nc3ccc4nc(NC5CC5)ccc4c3P(C)(C)=O)n2)c(OC)cc1N1CCC(N2CCN(CCc3cc(F)c([C@H]4CCC(=O)NC4=O)c(F)c3)CC2)CC1]

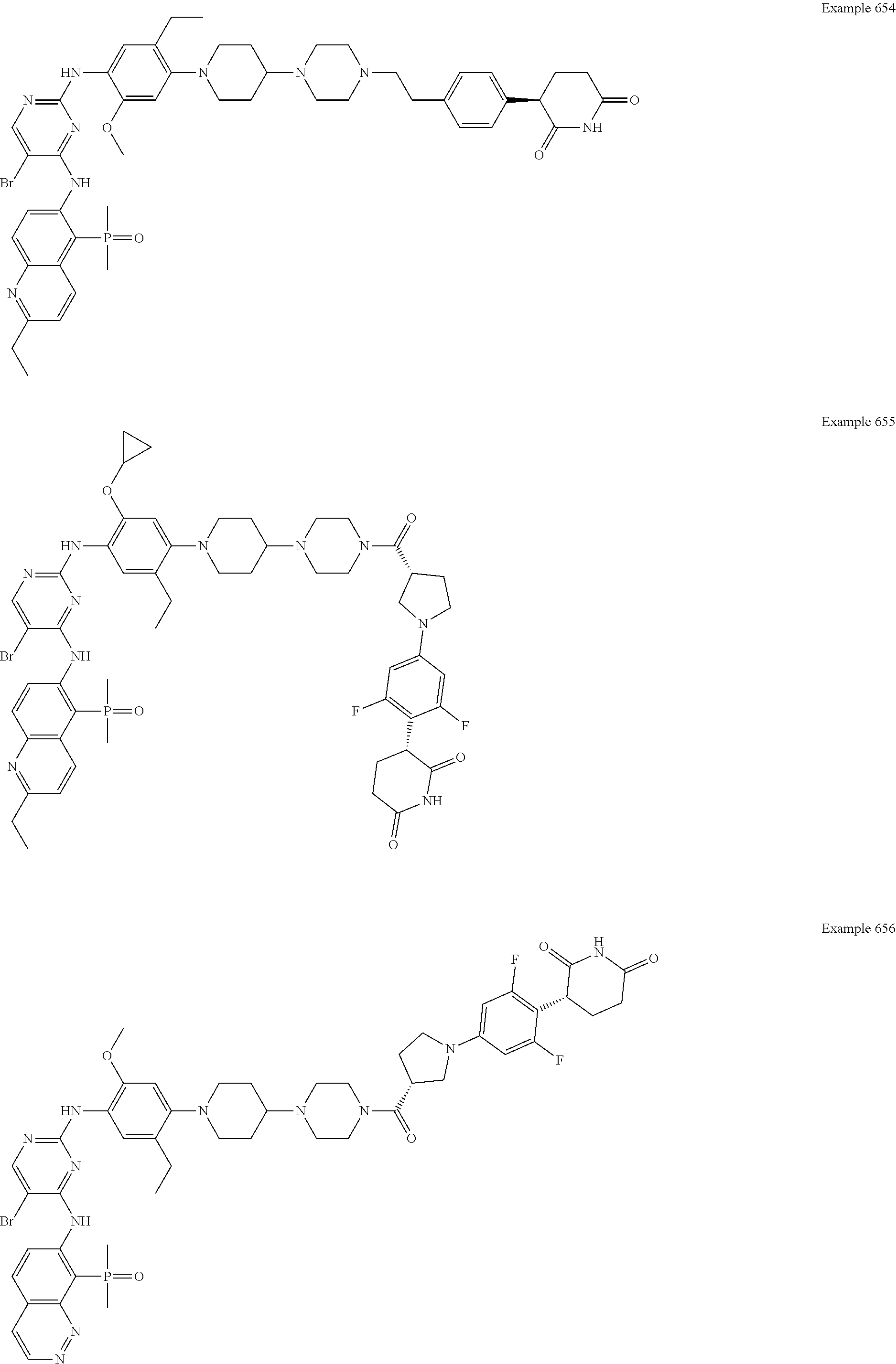 [SMILES: CCc1ccc2c(P(C)(C)=O)c(Nc3nc(Nc4cc(CC)c(N5CCC(N6CCN(CCc7ccc([C@H]8CCC(=O)NC8=O)cc7)CC6)CC5)cc4OC)ncc3Br)ccc2n1 ; CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC4CC4)ncc3Br)ccc2n1 ; CCc1cc(Nc2ncc(Br)c(Nc3ccc4ccnnc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

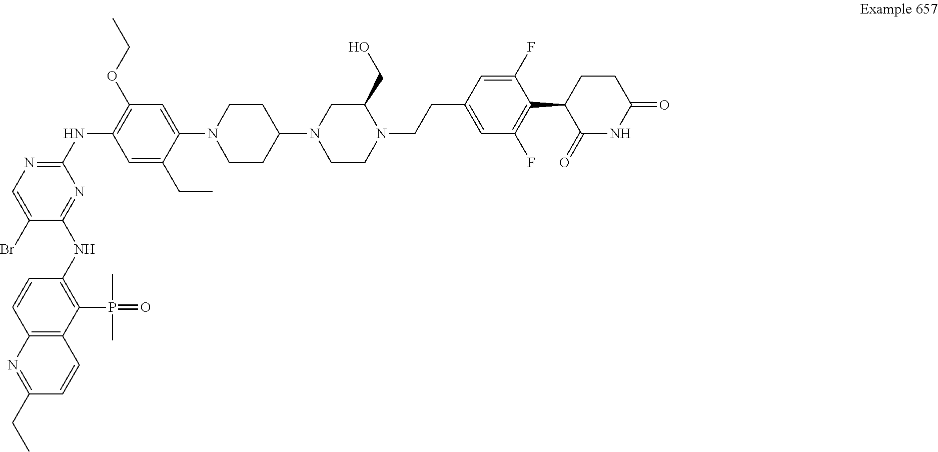 [SMILES: CCOc1cc(N2CCC(N3CCN(CCc4cc(F)c([C@H]5CCC(=O)NC5=O)c(F)c4)[C@H](CO)C3)CC2)c(CC)cc1Nc1ncc(Br)c(Nc2ccc3nc(CC)ccc3c2P(C)(C)=O)n1]

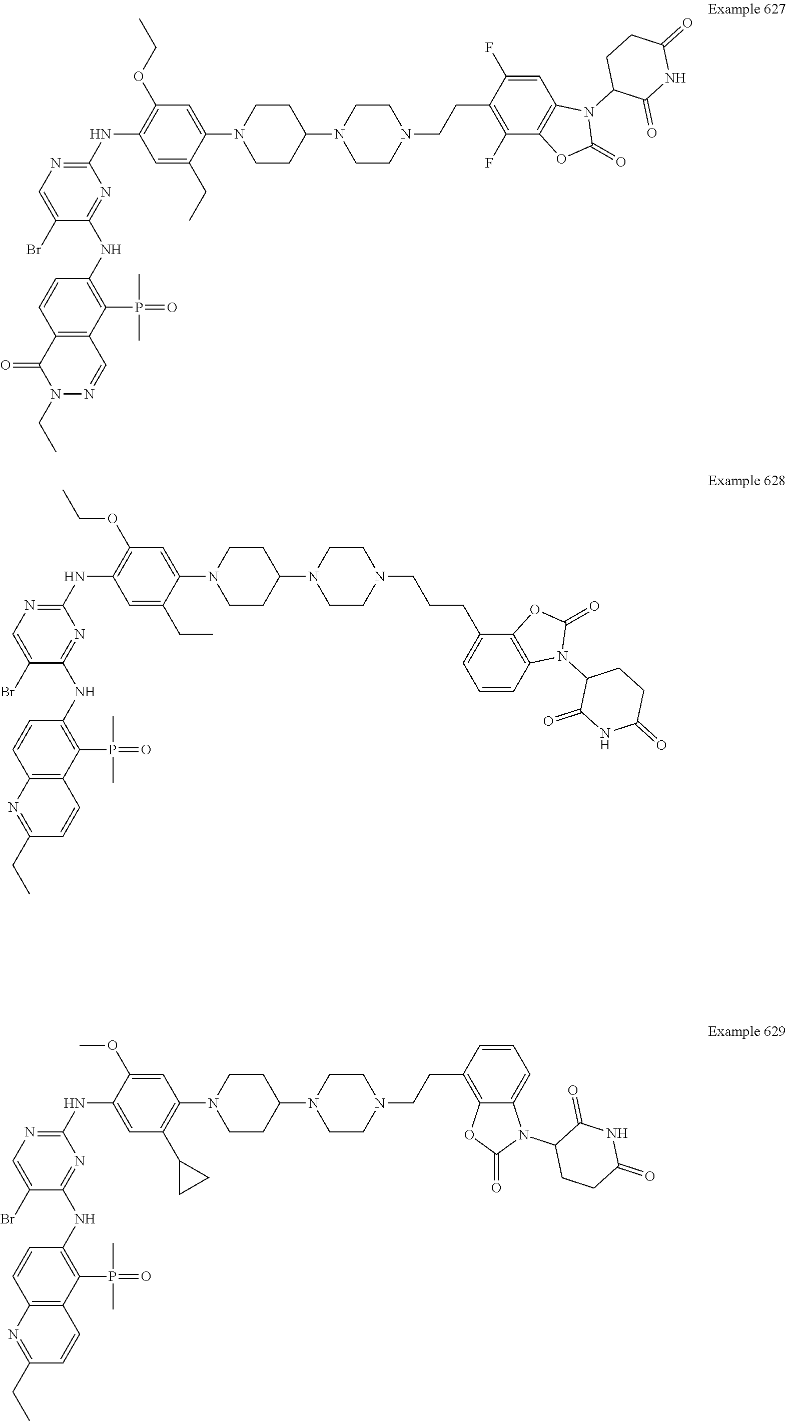

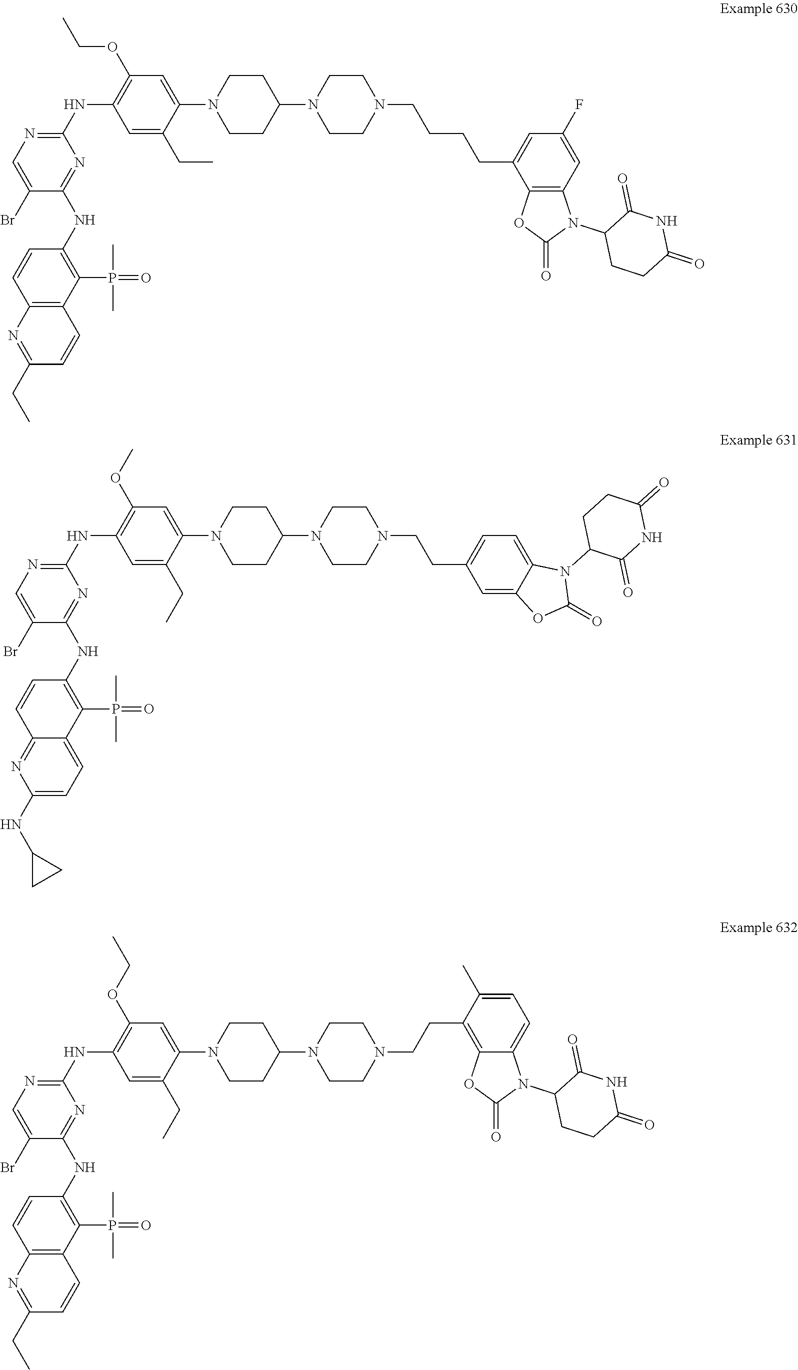 [SMILES: CCOc1cc(N2CCC(N3CCN(CCCCc4cc(F)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCc1cc(Nc2ncc(Br)c(Nc3ccc4nc(NC5CC5)ccc4c3P(C)(C)=O)n2)c(OC)cc1N1CCC(N2CCN(CCc3ccc4c(c3)oc(=O)n4C3CCC(=O)NC3=O)CC2)CC1 ; CCOc1cc(N2CCC(N3CCN(CCc4c(C)ccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

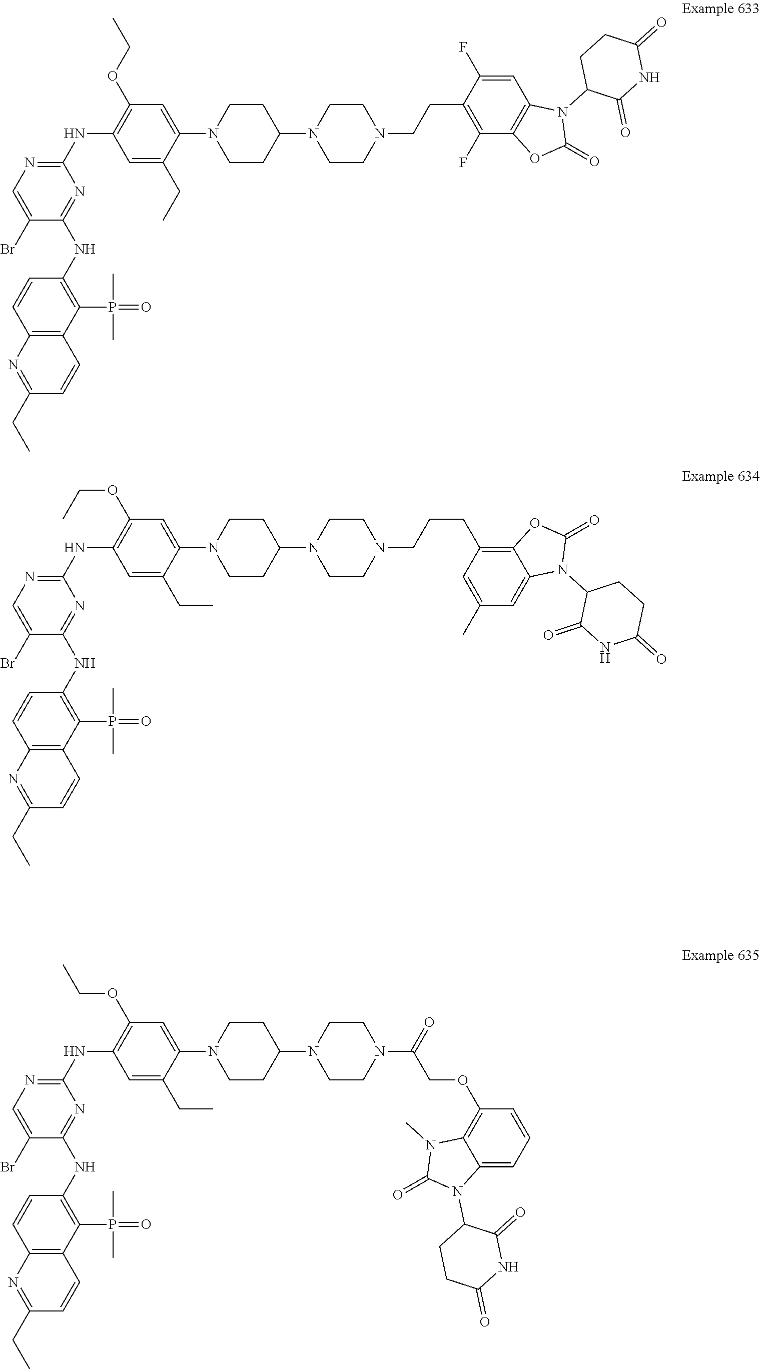 [SMILES: CCOc1cc(N2CCC(N3CCN(CCc4c(F)cc5c(oc(=O)n5C5CCC(=O)NC5=O)c4F)CC3)CC2)c(CC)cc1Nc1ncc(Br)c(Nc2ccc3nc(CC)ccc3c2P(C)(C)=O)n1 ; CCOc1cc(N2CCC(N3CCN(CCCc4cc(C)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(C(=O)COc4cccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

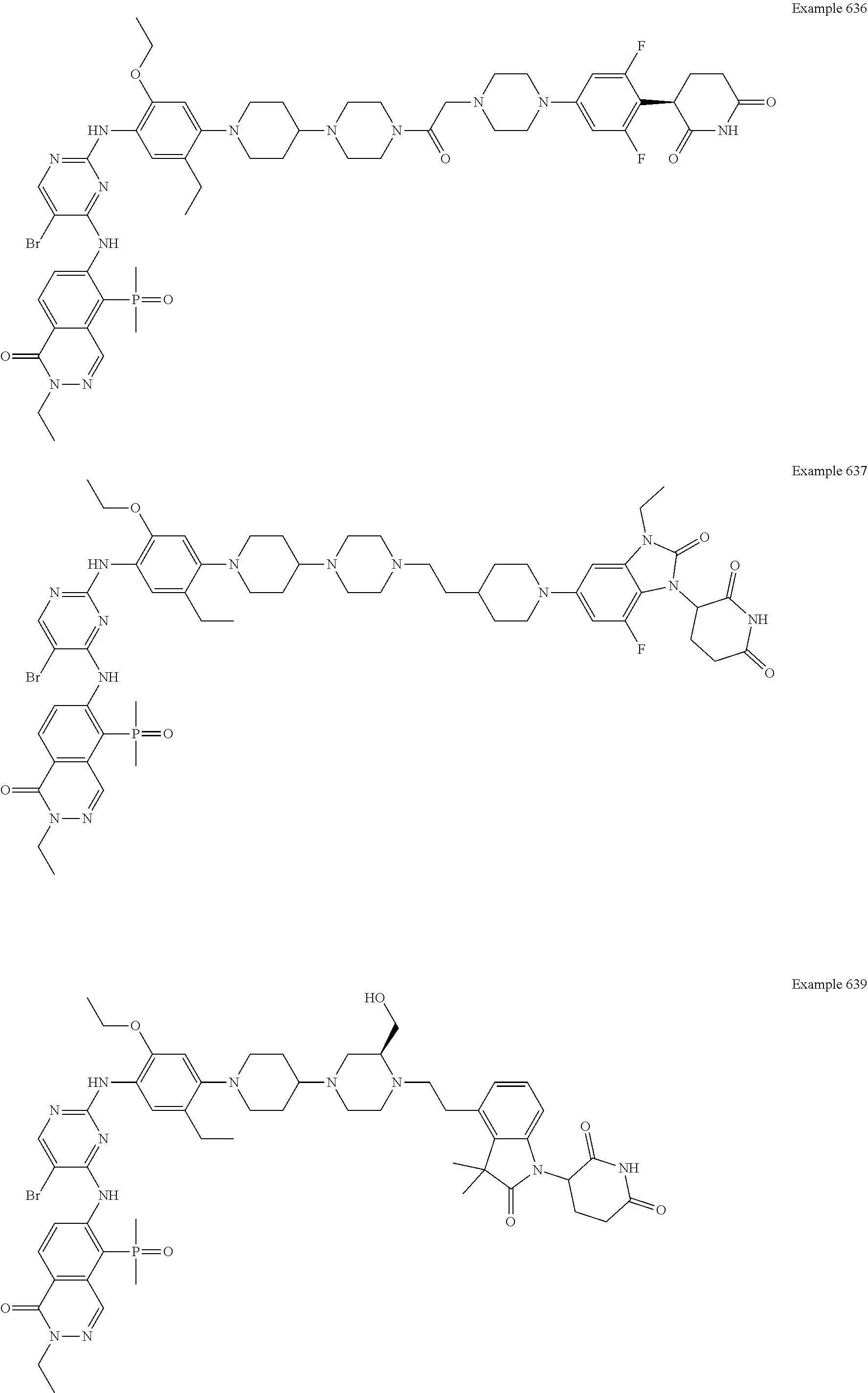 [SMILES: CCOc1cc(N2CCC(N3CCN(C(=O)CN4CCN(c5cc(F)c([C@H]6CCC(=O)NC6=O)c(F)c5)CC4)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCC4CCN(c5cc(F)c6c(c5)n(CC)c(=O)n6C5CCC(=O)NC5=O)CC4)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCc4cccc5c4C(C)(C)C(=O)N5C4CCC(=O)NC4=O)[C@H](CO)C3)CC2)c(CC)cc1Nc1ncc(Br)c(Nc2ccc3c(=O)n(CC)ncc3c2P(C)(C)=O)n1]

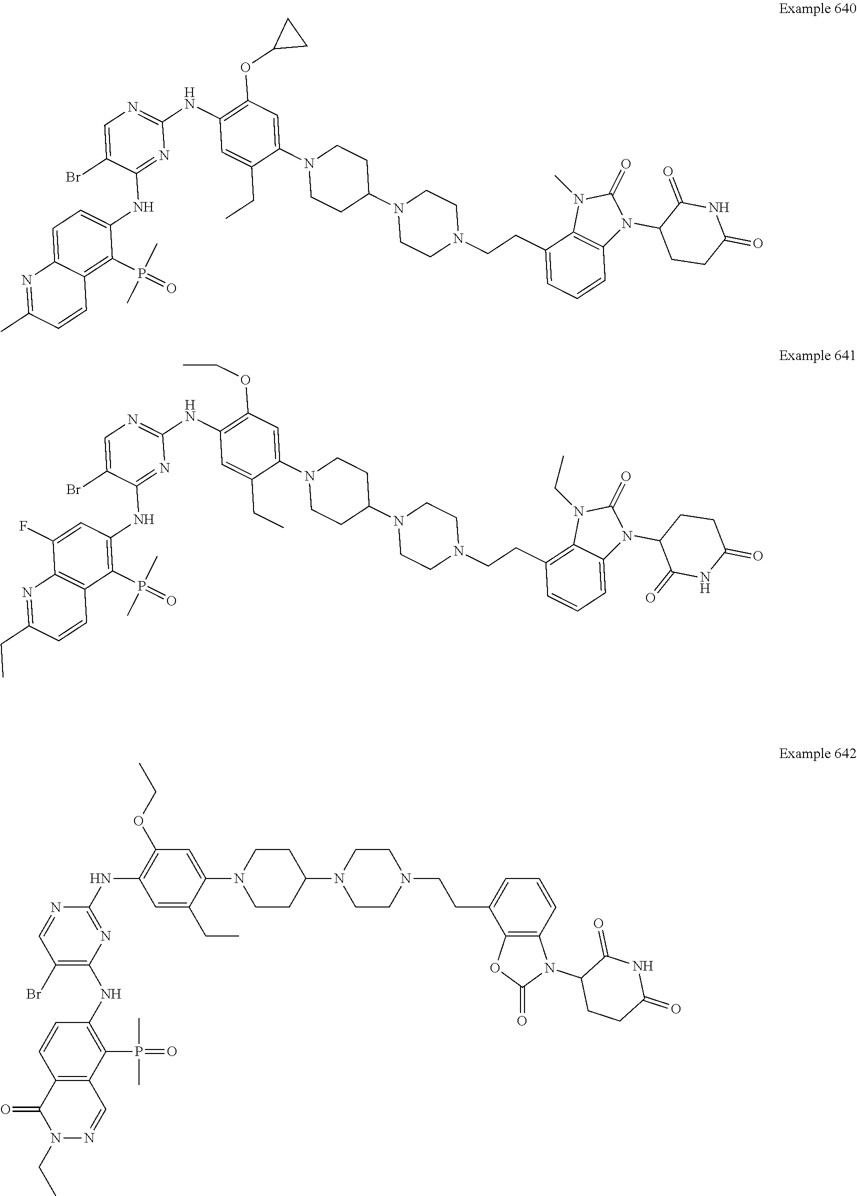 [SMILES: CCc1cc(Nc2ncc(Br)c(Nc3ccc4nc(C)ccc4c3P(C)(C)=O)n2)c(OC2CC2)cc1N1CCC(N2CCN(CCc3cccc4c3n(C)c(=O)n4C3CCC(=O)NC3=O)CC2)CC1 ; CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2cc(F)c3nc(CC)ccc3c2P(C)(C)=O)n1 ; CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3c(=O)n(CC)ncc3c2P(C)(C)=O)n1]

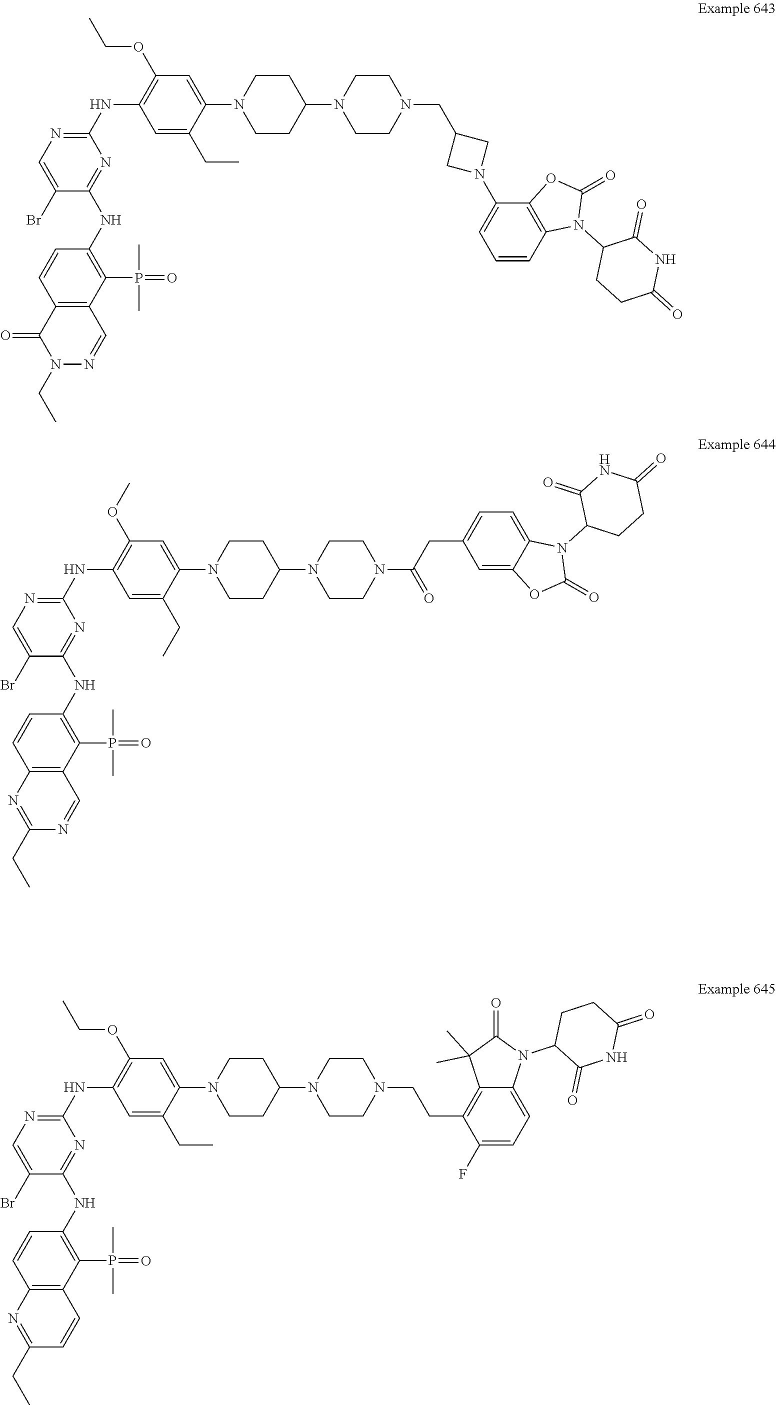 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3c(=O)n(CC)ncc3c2P(C)(C)=O)n1 ; CCc1ncc2c(P(C)(C)=O)c(Nc3nc(Nc4cc(CC)c(N5CCC(N6CCN(C(=O)Cc7ccc8c(c7)oc(=O)n8C7CCC(=O)NC7=O)CC6)CC5)cc4OC)ncc3Br)ccc2n1 ; CCOc1cc(N2CCC(N3CCN(CCc4c(F)ccc5c4C(C)(C)C(=O)N5C4CCC(=O)NC4=O)CC3)CC2)c(CC)cc1Nc1ncc(Br)c(Nc2ccc3nc(CC)ccc3c2P(C)(C)=O)n1]

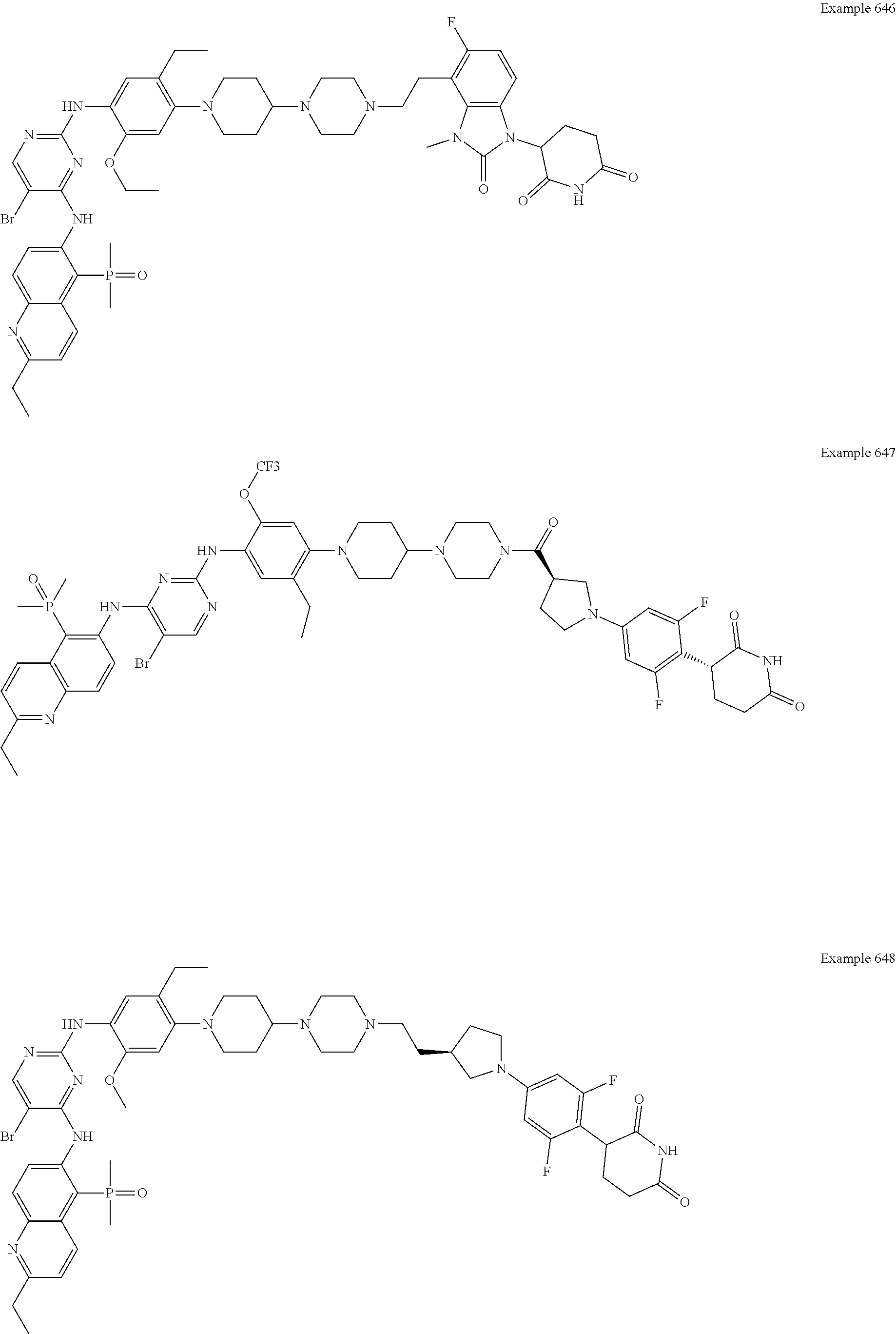 [SMILES: CCOc1cc(N2CCC(N3CCN(CCc4c(F)ccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1 ; CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC(F)(F)F)ncc3Br)ccc2n1 ; CCc1ccc2c(P(C)(C)=O)c(Nc3nc(Nc4cc(CC)c(N5CCC(N6CCN(CC[C@H]7CCN(c8cc(F)c(C9CCC(=O)NC9=O)c(F)c8)C7)CC6)CC5)cc4OC)ncc3Br)ccc2n1]

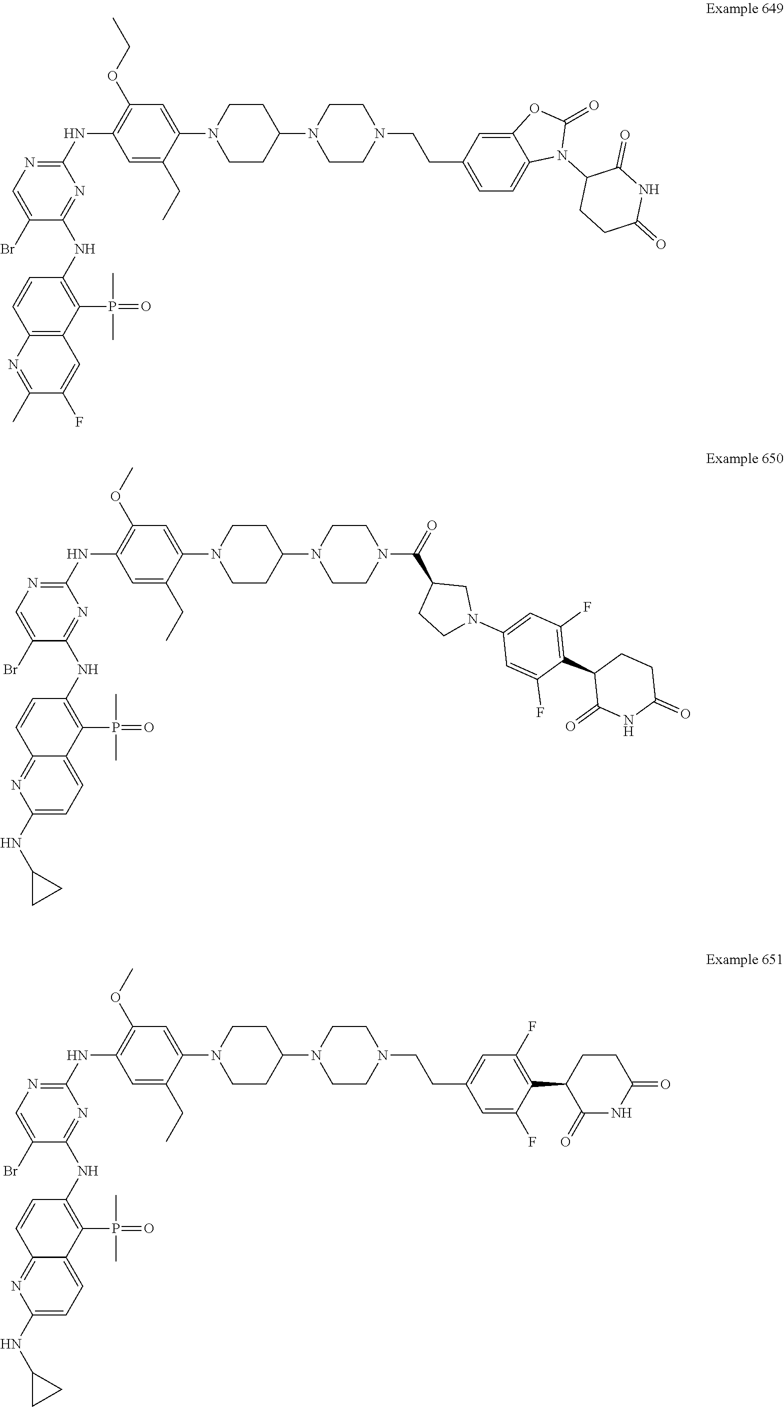 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2ccc3nc(C)c(F)cc3c2P(C)(C)=O)n1 ; CCc1cc(Nc2ncc(Br)c(Nc3ccc4nc(NC5CC5)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; CCc1cc(Nc2ncc(Br)c(Nc3ccc4nc(NC5CC5)ccc4c3P(C)(C)=O)n2)c(OC)cc1N1CCC(N2CCN(CCc3cc(F)c([C@H]4CCC(=O)NC4=O)c(F)c3)CC2)CC1]

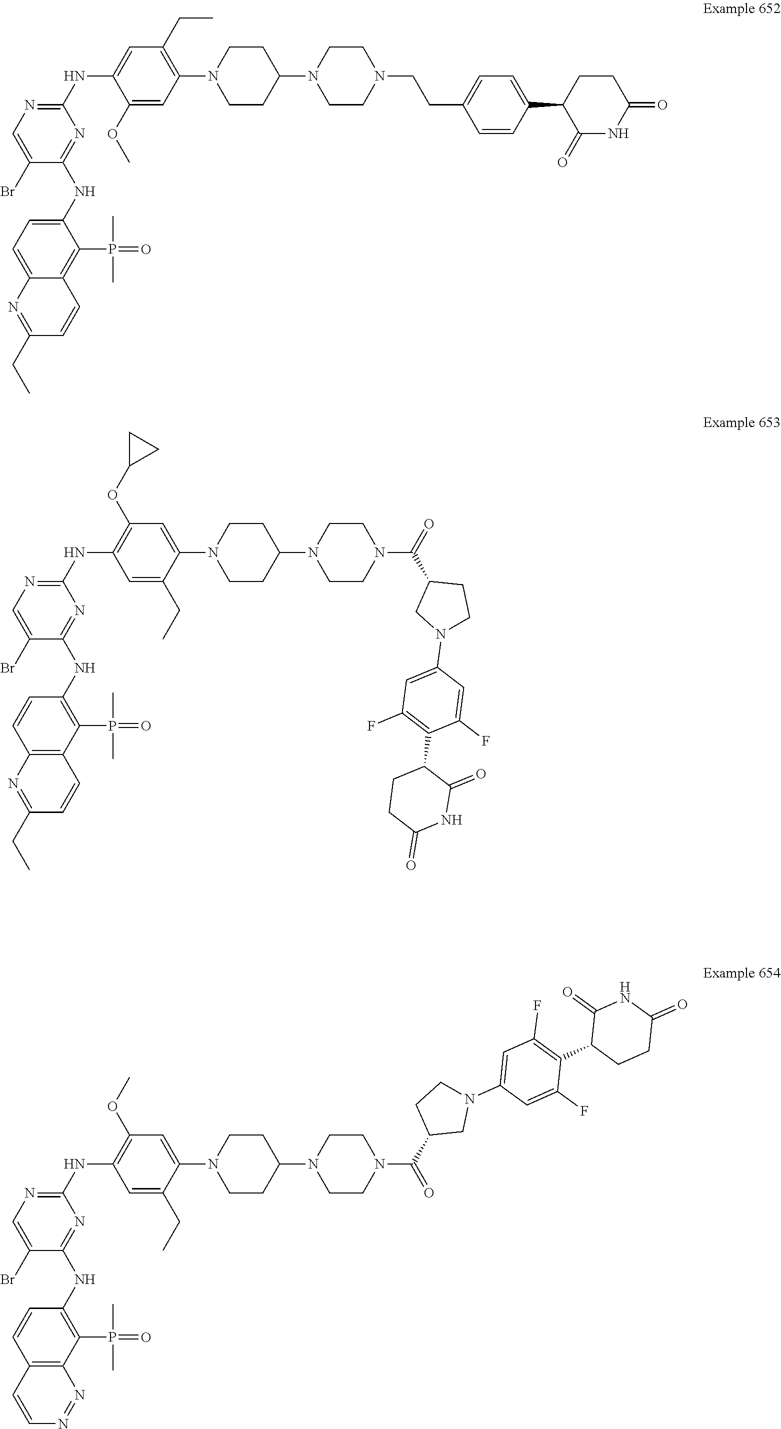 [SMILES: CCc1ccc2c(P(C)(C)=O)c(Nc3nc(Nc4cc(CC)c(N5CCC(N6CCN(CCc7ccc([C@H]8CCC(=O)NC8=O)cc7)CC6)CC5)cc4OC)ncc3Br)ccc2n1 ; CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC4CC4)ncc3Br)ccc2n1 ; CCc1cc(Nc2ncc(Br)c(Nc3ccc4ccnnc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

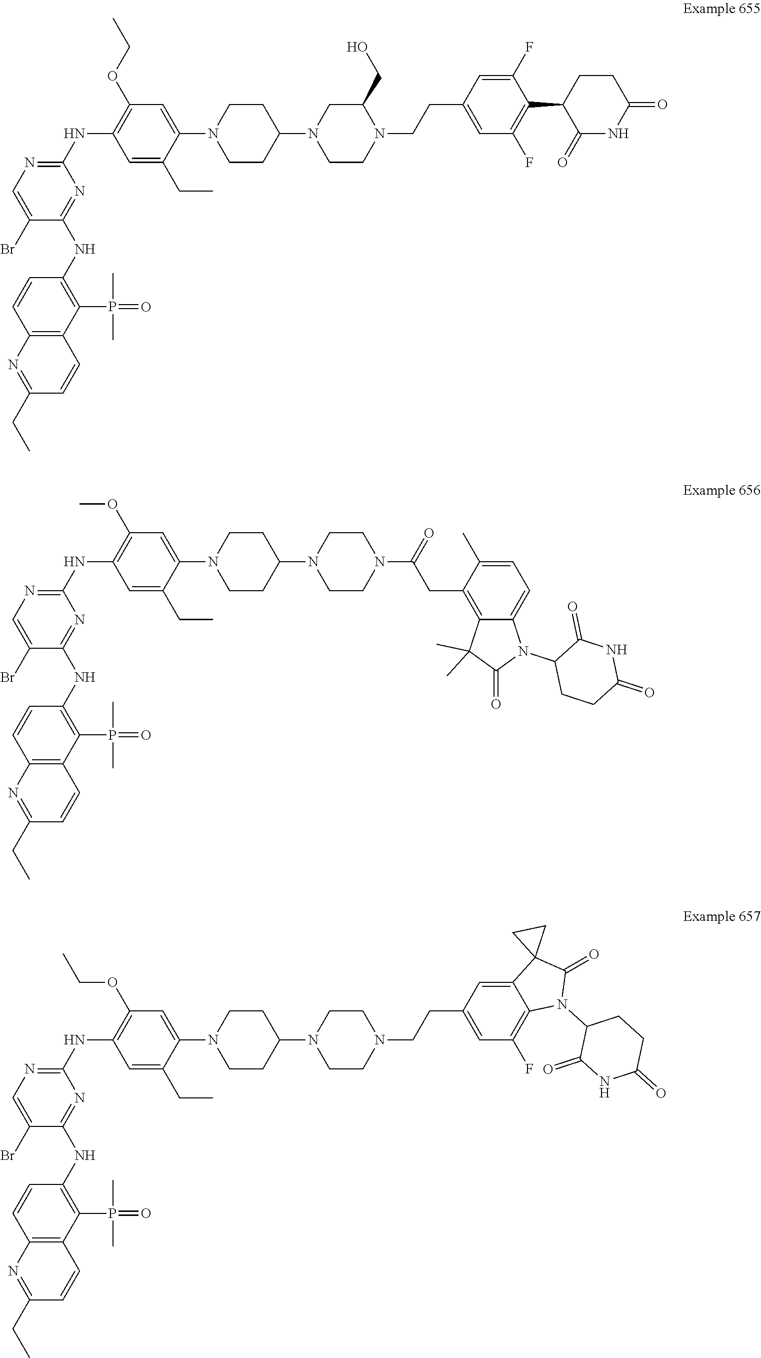 [SMILES: CCOc1cc(N2CCC(N3CCN(CCc4cc(F)c([C@H]5CCC(=O)NC5=O)c(F)c4)[C@H](CO)C3)CC2)c(CC)cc1Nc1ncc(Br)c(Nc2ccc3nc(CC)ccc3c2P(C)(C)=O)n1 ; CCc1ccc2c(P(C)(C)=O)c(Nc3nc(Nc4cc(CC)c(N5CCC(N6CCN(C(=O)Cc7c(C)ccc8c7C(C)(C)C(=O)N8C7CCC(=O)NC7=O)CC6)CC5)cc4OC)ncc3Br)ccc2n1 ; CCOc1cc(N2CCC(N3CCN(CCc4cc(F)c5c(c4)C4(CC4)C(=O)N5C4CCC(=O)NC4=O)CC3)CC2)c(CC)cc1Nc1ncc(Br)c(Nc2ccc3nc(CC)ccc3c2P(C)(C)=O)n1]

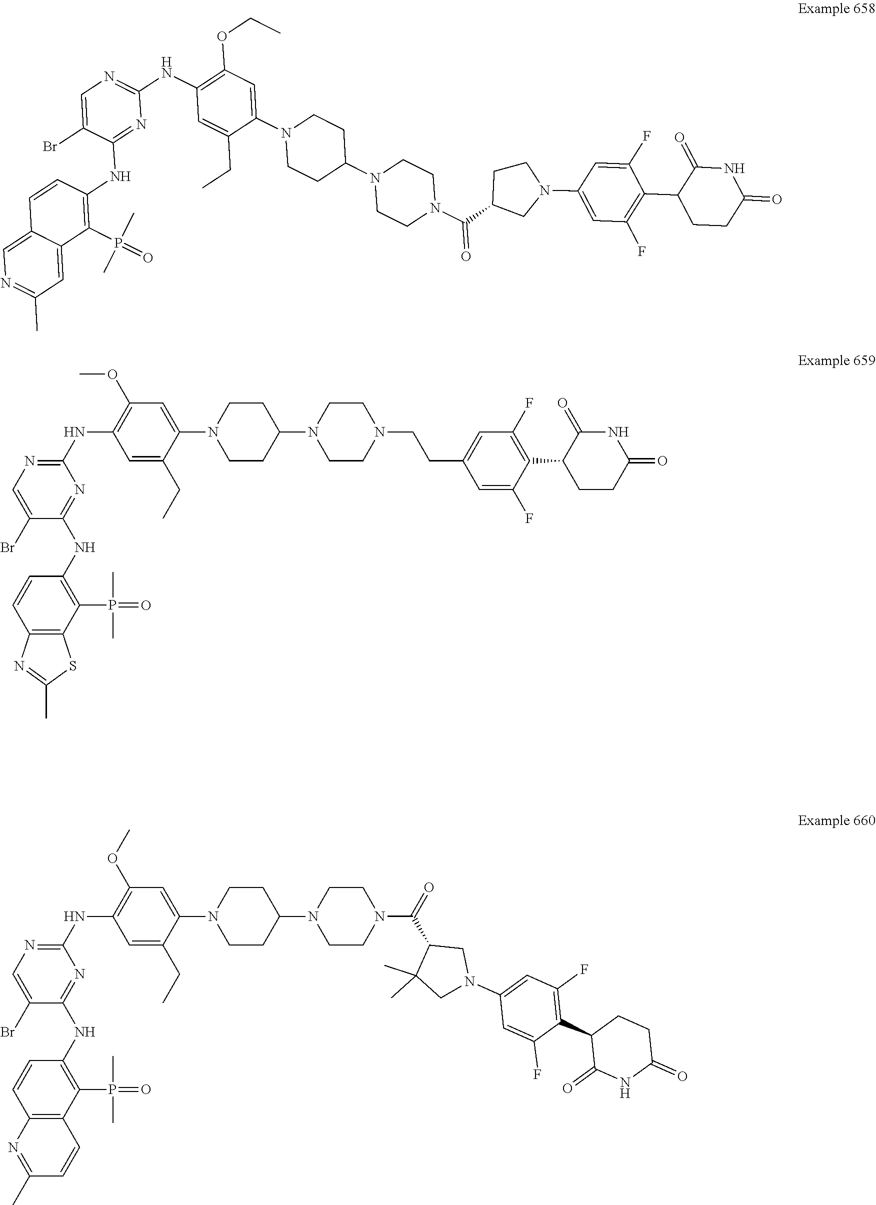 [SMILES: CCOc1cc(N2CCC(N3CCN(C(=O)[C@@H]4CCN(c5cc(F)c(C6CCC(=O)NC6=O)c(F)c5)C4)CC3)CC2)c(CC)cc1Nc1ncc(Br)c(Nc2ccc3cnc(C)cc3c2P(C)(C)=O)n1 ; CCc1cc(Nc2ncc(Br)c(Nc3ccc4nc(C)sc4c3P(C)(C)=O)n2)c(OC)cc1N1CCC(N2CCN(CCc3cc(F)c([C@H]4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CCN(C(=O)[C@@H]3CN(c4cc(F)c([C@H]5CCC(=O)NC5=O)c(F)c4)CC3(C)C)CC2)CC1]

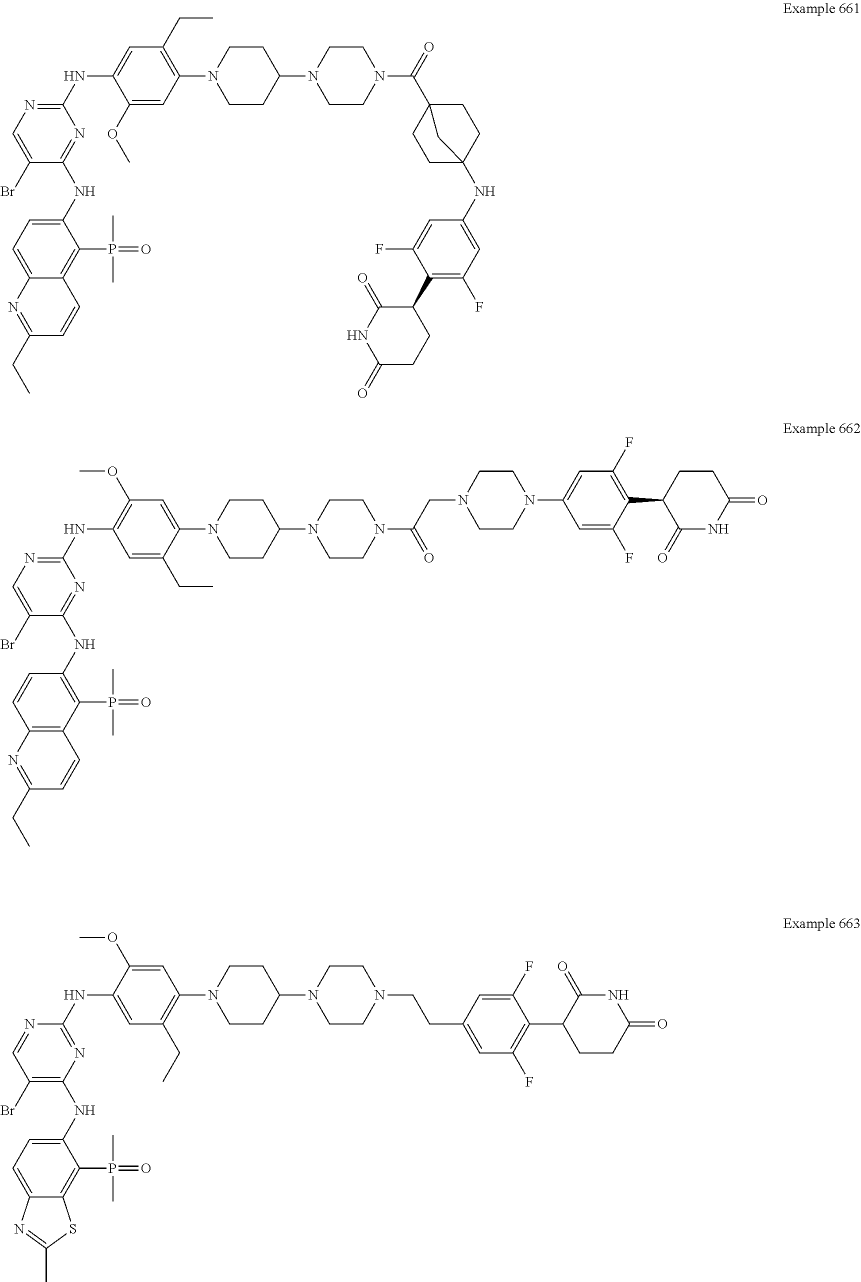 [SMILES: CCc1ccc2c(P(C)(C)=O)c(Nc3nc(Nc4cc(CC)c(N5CCC(N6CCN(C(=O)C78CCC(Nc9cc(F)c([C@H]%10CCC(=O)NC%10=O)c(F)c9)(CC7)C8)CC6)CC5)cc4OC)ncc3Br)ccc2n1 ; CCc1ccc2c(P(C)(C)=O)c(Nc3nc(Nc4cc(CC)c(N5CCC(N6CCN(C(=O)CN7CCN(c8cc(F)c([C@H]9CCC(=O)NC9=O)c(F)c8)CC7)CC6)CC5)cc4OC)ncc3Br)ccc2n1 ; CCc1cc(Nc2ncc(Br)c(Nc3ccc4nc(C)sc4c3P(C)(C)=O)n2)c(OC)cc1N1CCC(N2CCN(CCc3cc(F)c(C4CCC(=O)NC4=O)c(F)c3)CC2)CC1]

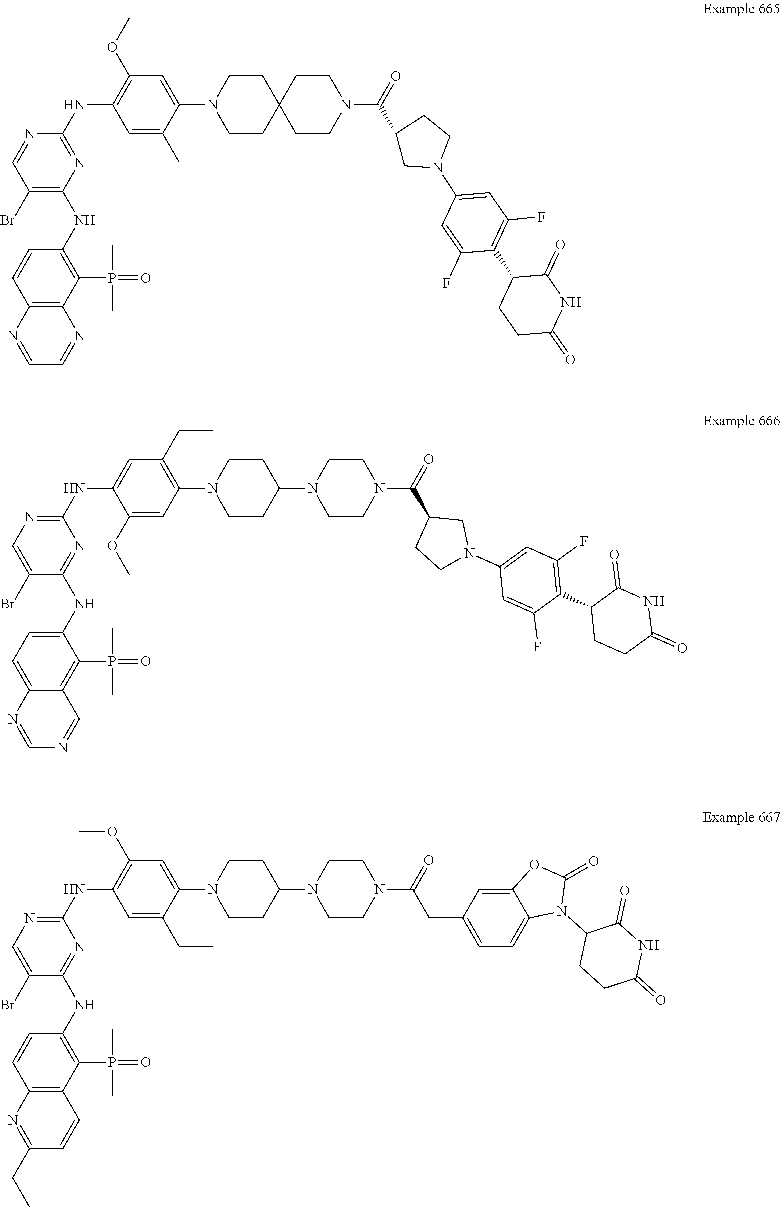 [SMILES: COc1cc(N2CCC3(CCN(C(=O)[C@@H]4CCN(c5cc(F)c([C@H]6CCC(=O)NC6=O)c(F)c5)C4)CC3)CC2)c(C)cc1Nc1ncc(Br)c(Nc2ccc3nccnc3c2P(C)(C)=O)n1 ; CCc1cc(Nc2ncc(Br)c(Nc3ccc4ncncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; CCc1ccc2c(P(C)(C)=O)c(Nc3nc(Nc4cc(CC)c(N5CCC(N6CCN(C(=O)Cc7ccc8c(c7)oc(=O)n8C7CCC(=O)NC7=O)CC6)CC5)cc4OC)ncc3Br)ccc2n1]

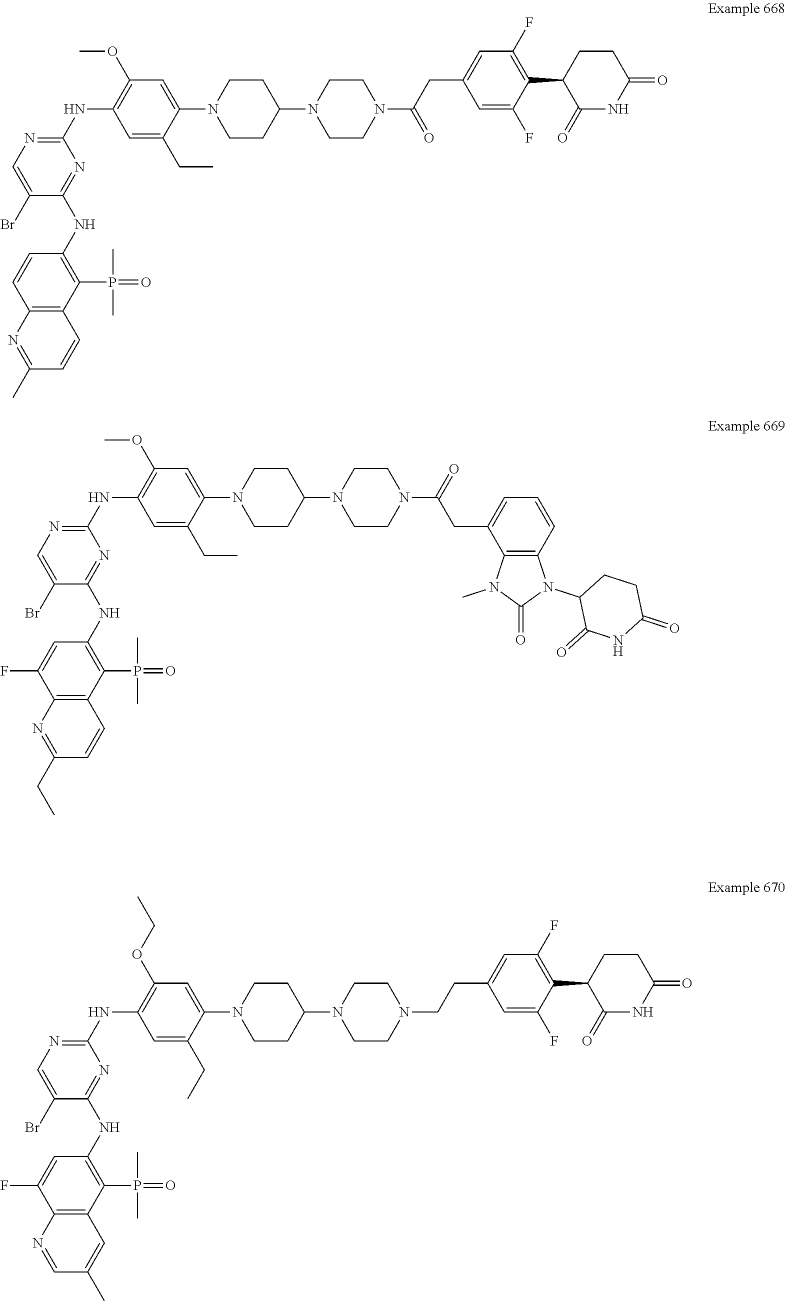 [SMILES: CCc1cc(Nc2ncc(Br)c(Nc3ccc4nc(C)ccc4c3P(C)(C)=O)n2)c(OC)cc1N1CCC(N2CCN(C(=O)Cc3cc(F)c([C@H]4CCC(=O)NC4=O)c(F)c3)CC2)CC1 ; CCc1ccc2c(P(C)(C)=O)c(Nc3nc(Nc4cc(CC)c(N5CCC(N6CCN(C(=O)Cc7cccc8c7n(C)c(=O)n8C7CCC(=O)NC7=O)CC6)CC5)cc4OC)ncc3Br)cc(F)c2n1 ; CCOc1cc(N2CCC(N3CCN(CCc4cc(F)c([C@H]5CCC(=O)NC5=O)c(F)c4)CC3)CC2)c(CC)cc1Nc1ncc(Br)c(Nc2cc(F)c3ncc(C)cc3c2P(C)(C)=O)n1]

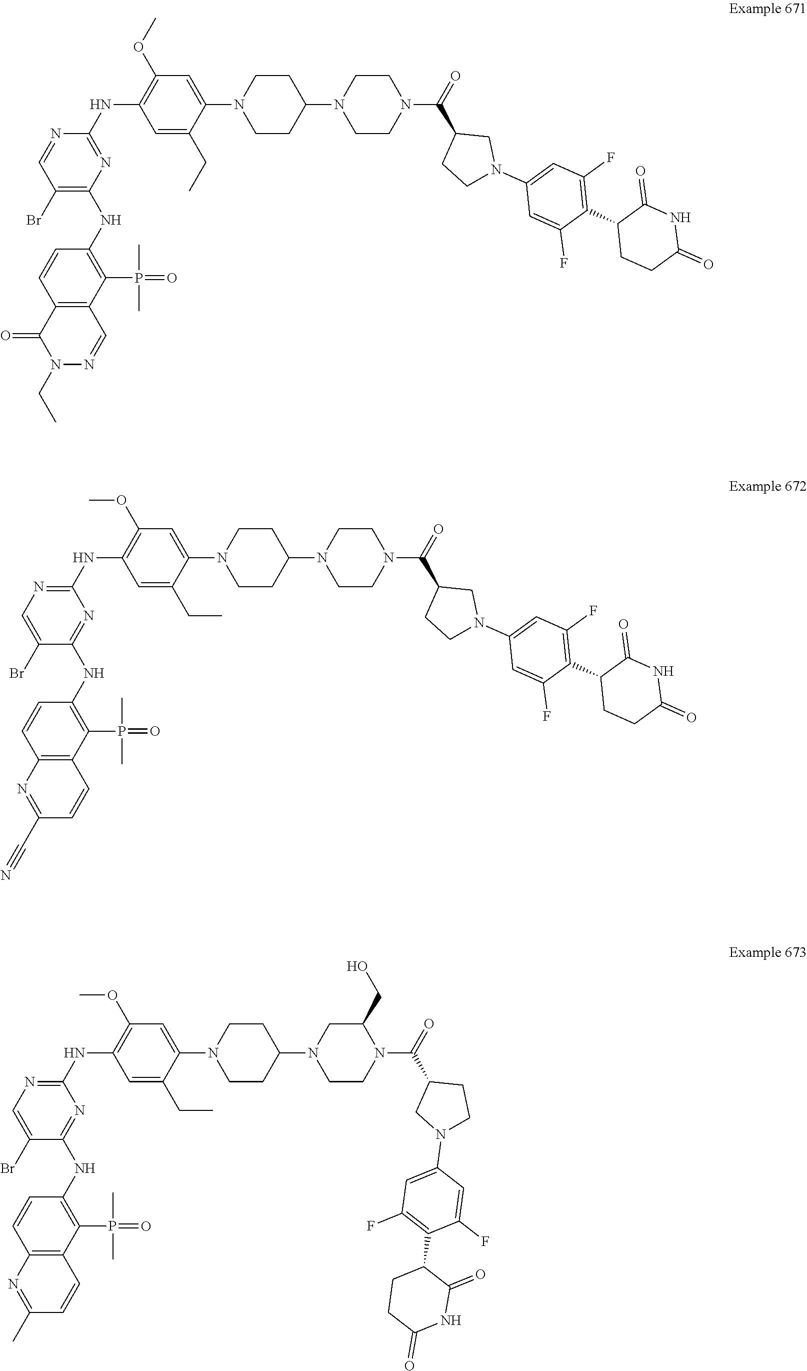 [SMILES: CCc1cc(Nc2ncc(Br)c(Nc3ccc4c(=O)n(CC)ncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; CCc1cc(Nc2ncc(Br)c(Nc3ccc4nc(C#N)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)[C@H](CO)C2)CC1]

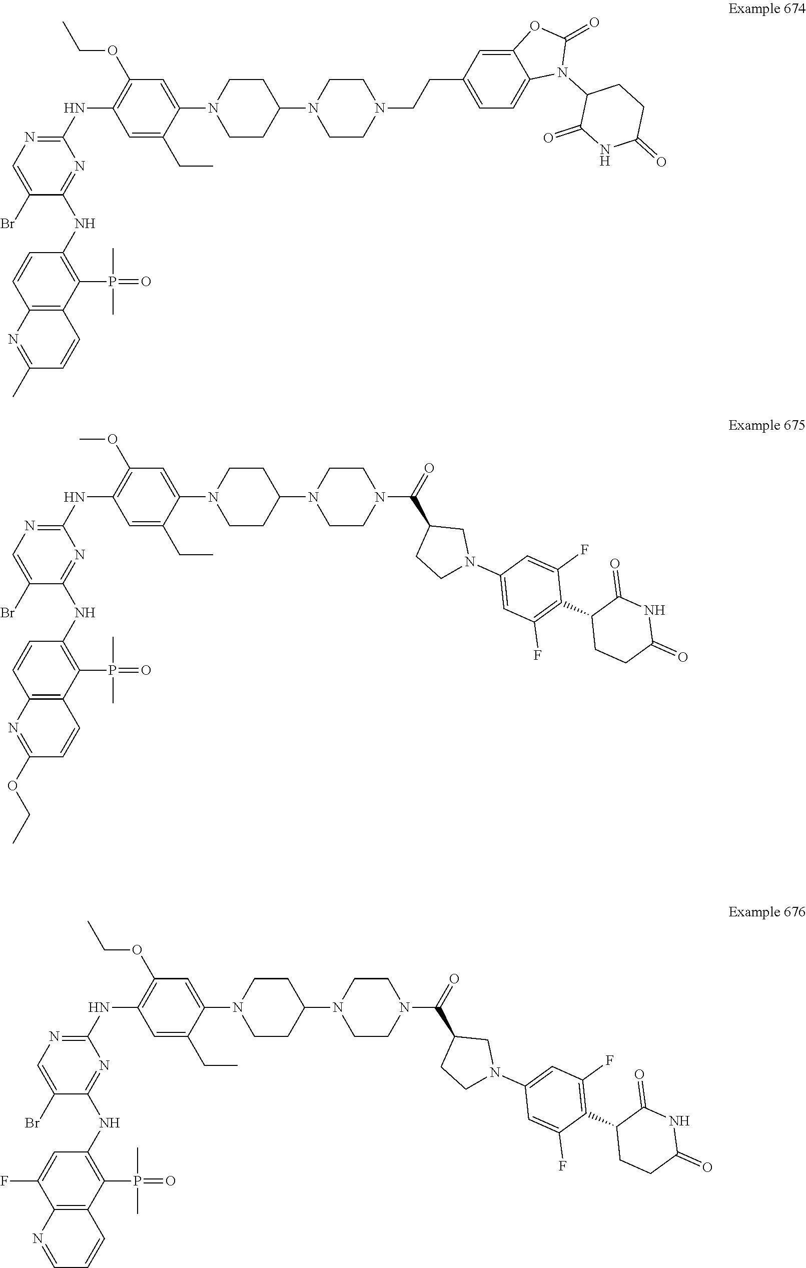 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2ccc3nc(C)ccc3c2P(C)(C)=O)n1 ; CCOc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1 ; CCOc1cc(N2CCC(N3CCN(C(=O)[C@@H]4CCN(c5cc(F)c([C@H]6CCC(=O)NC6=O)c(F)c5)C4)CC3)CC2)c(CC)cc1Nc1ncc(Br)c(Nc2cc(F)c3ncccc3c2P(C)(C)=O)n1]

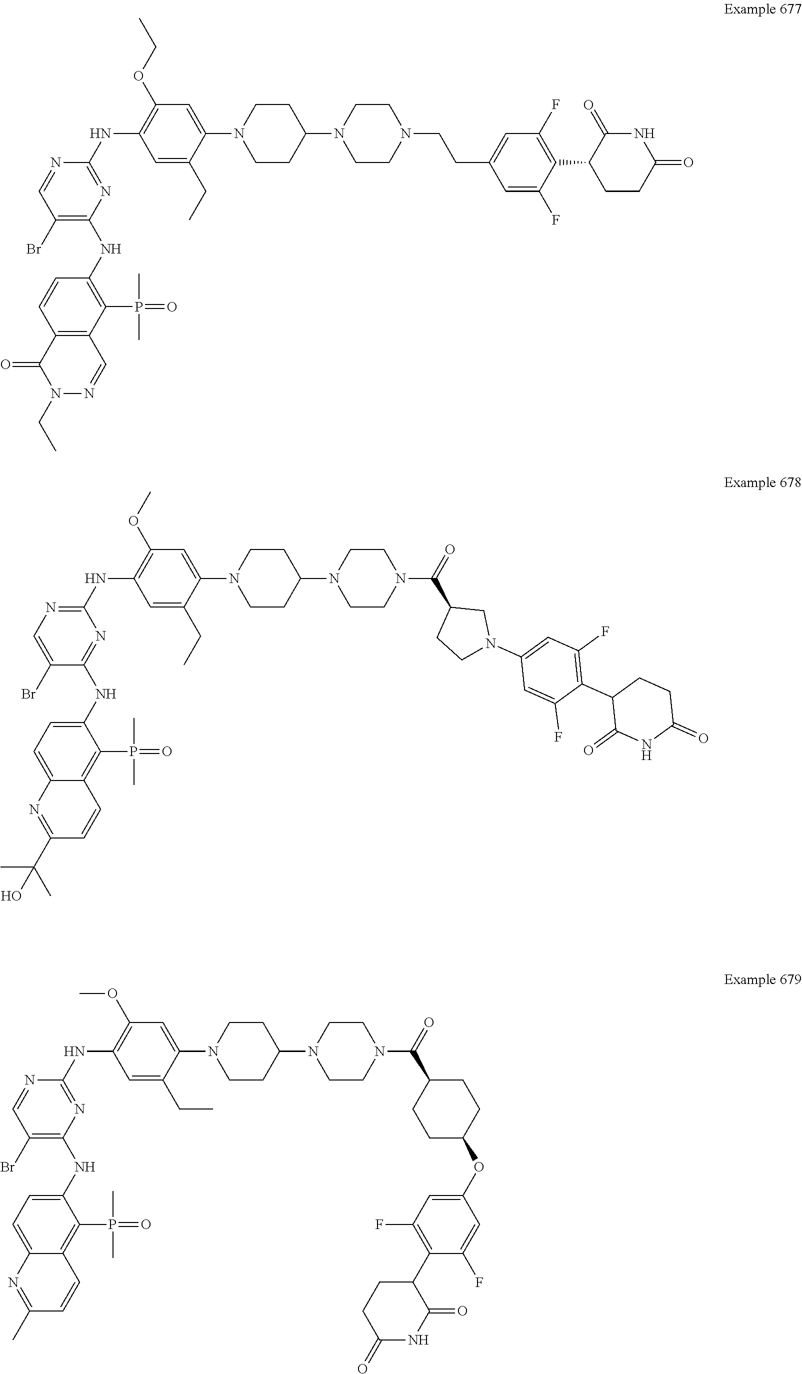 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3c(=O)n(CC)ncc3c2P(C)(C)=O)n1 ; CCc1cc(Nc2ncc(Br)c(Nc3ccc4nc(C(C)(C)O)ccc4c3P(C)(C)=O)n2)c(OC)cc1N1CCC(N2CCN(C(=O)[C@@H]3CCN(c4cc(F)c(C5CCC(=O)NC5=O)c(F)c4)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)[C@H]3CC[C@@H](Oc4cc(F)c(C5CCC(=O)NC5=O)c(F)c4)CC3)CC2)CC1]

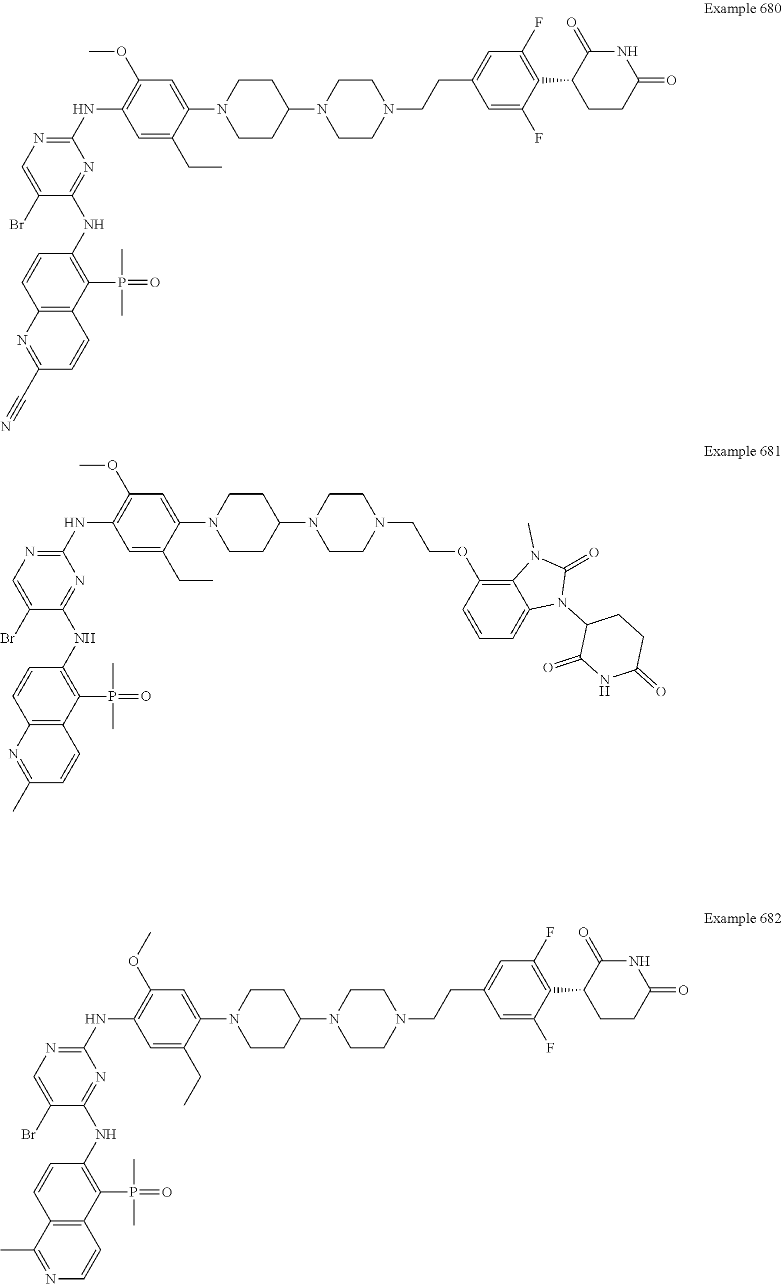 [SMILES: CCc1cc(Nc2ncc(Br)c(Nc3ccc4nc(C#N)ccc4c3P(C)(C)=O)n2)c(OC)cc1N1CCC(N2CCN(CCc3cc(F)c([C@H]4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CCN(CCOc3cccc4c3n(C)c(=O)n4C3CCC(=O)NC3=O)CC2)CC1 ; CCc1cc(Nc2ncc(Br)c(Nc3ccc4c(C)nccc4c3P(C)(C)=O)n2)c(OC)cc1N1CCC(N2CCN(CCc3cc(F)c([C@H]4CCC(=O)NC4=O)c(F)c3)CC2)CC1]

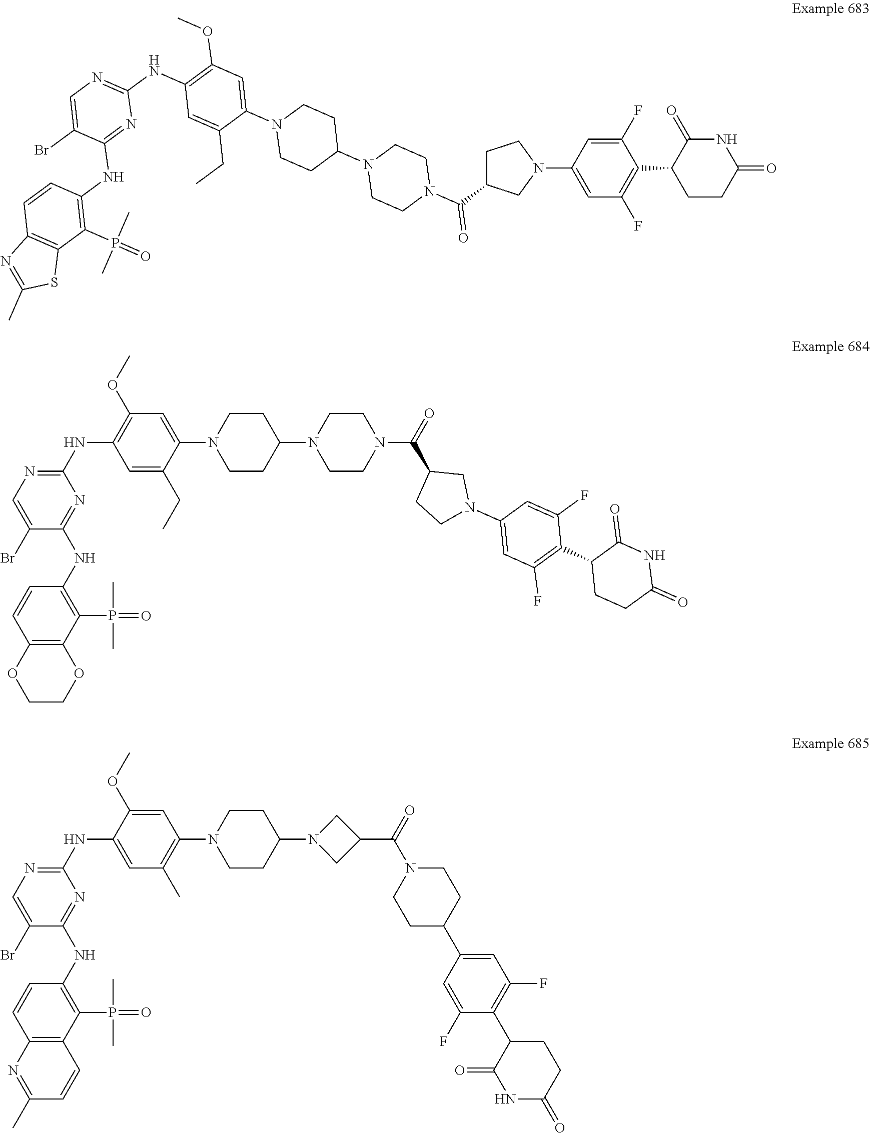 [SMILES: CCc1cc(Nc2ncc(Br)c(Nc3ccc4nc(C)sc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; CCc1cc(Nc2ncc(Br)c(Nc3ccc4c(c3P(C)(C)=O)OCCO4)n2)c(OC)cc1N1CCC(N2CCN(C(=O)[C@@H]3CCN(c4cc(F)c([C@H]5CCC(=O)NC5=O)c(F)c4)C3)CC2)CC1 ; COc1cc(N2CCC(N3CC(C(=O)N4CCC(c5cc(F)c(C6CCC(=O)NC6=O)c(F)c5)CC4)C3)CC2)c(C)cc1Nc1ncc(Br)c(Nc2ccc3nc(C)ccc3c2P(C)(C)=O)n1]

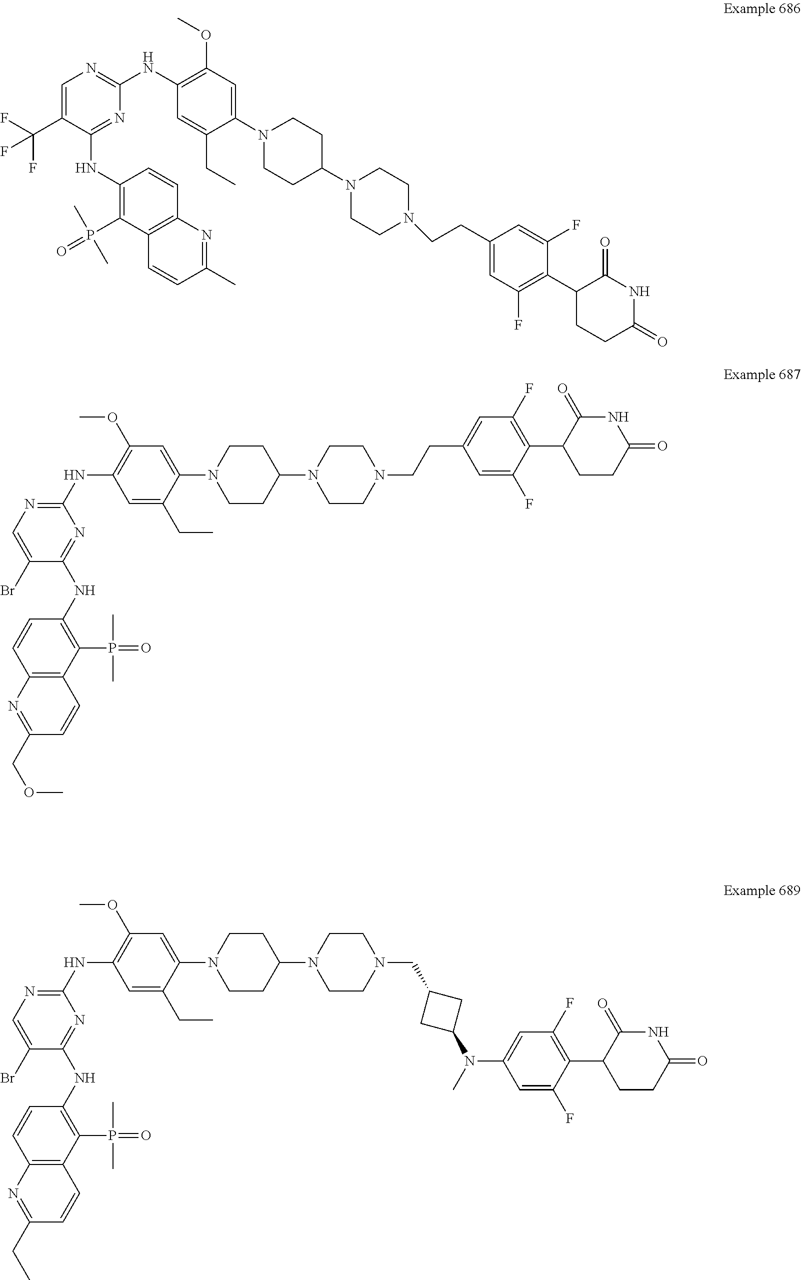 [SMILES: CCc1cc(Nc2ncc(C(F)(F)F)c(Nc3ccc4nc(C)ccc4c3P(C)(C)=O)n2)c(OC)cc1N1CCC(N2CCN(CCc3cc(F)c(C4CCC(=O)NC4=O)c(F)c3)CC2)CC1 ; CCc1cc(Nc2ncc(Br)c(Nc3ccc4nc(COC)ccc4c3P(C)(C)=O)n2)c(OC)cc1N1CCC(N2CCN(CCc3cc(F)c(C4CCC(=O)NC4=O)c(F)c3)CC2)CC1 ; CCc1ccc2c(P(C)(C)=O)c(Nc3nc(Nc4cc(CC)c(N5CCC(N6CCN(C[C@H]7C[C@H](N(C)c8cc(F)c(C9CCC(=O)NC9=O)c(F)c8)C7)CC6)CC5)cc4OC)ncc3Br)ccc2n1]

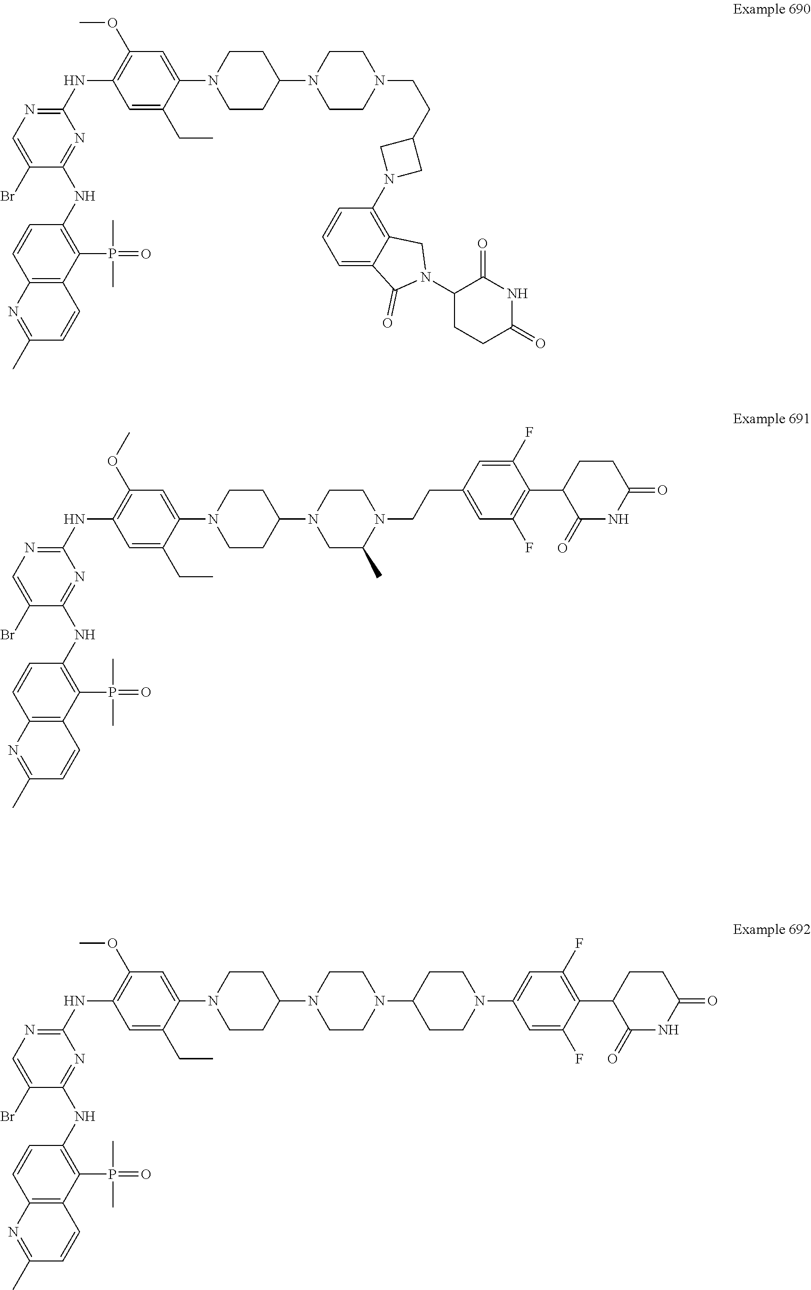 [SMILES: CCc1cc(Nc2ncc(Br)c(Nc3ccc4nc(C)ccc4c3P(C)(C)=O)n2)c(OC)cc1N1CCC(N2CCN(CCC3CN(c4cccc5c4CN(C4CCC(=O)NC4=O)C5=O)C3)CC2)CC1 ; CCc1cc(Nc2ncc(Br)c(Nc3ccc4nc(C)ccc4c3P(C)(C)=O)n2)c(OC)cc1N1CCC(N2CCN(CCc3cc(F)c(C4CCC(=O)NC4=O)c(F)c3)[C@@H](C)C2)CC1 ; CCc1cc(Nc2ncc(Br)c(Nc3ccc4nc(C)ccc4c3P(C)(C)=O)n2)c(OC)cc1N1CCC(N2CCN(C3CCN(c4cc(F)c(C5CCC(=O)NC5=O)c(F)c4)CC3)CC2)CC1]

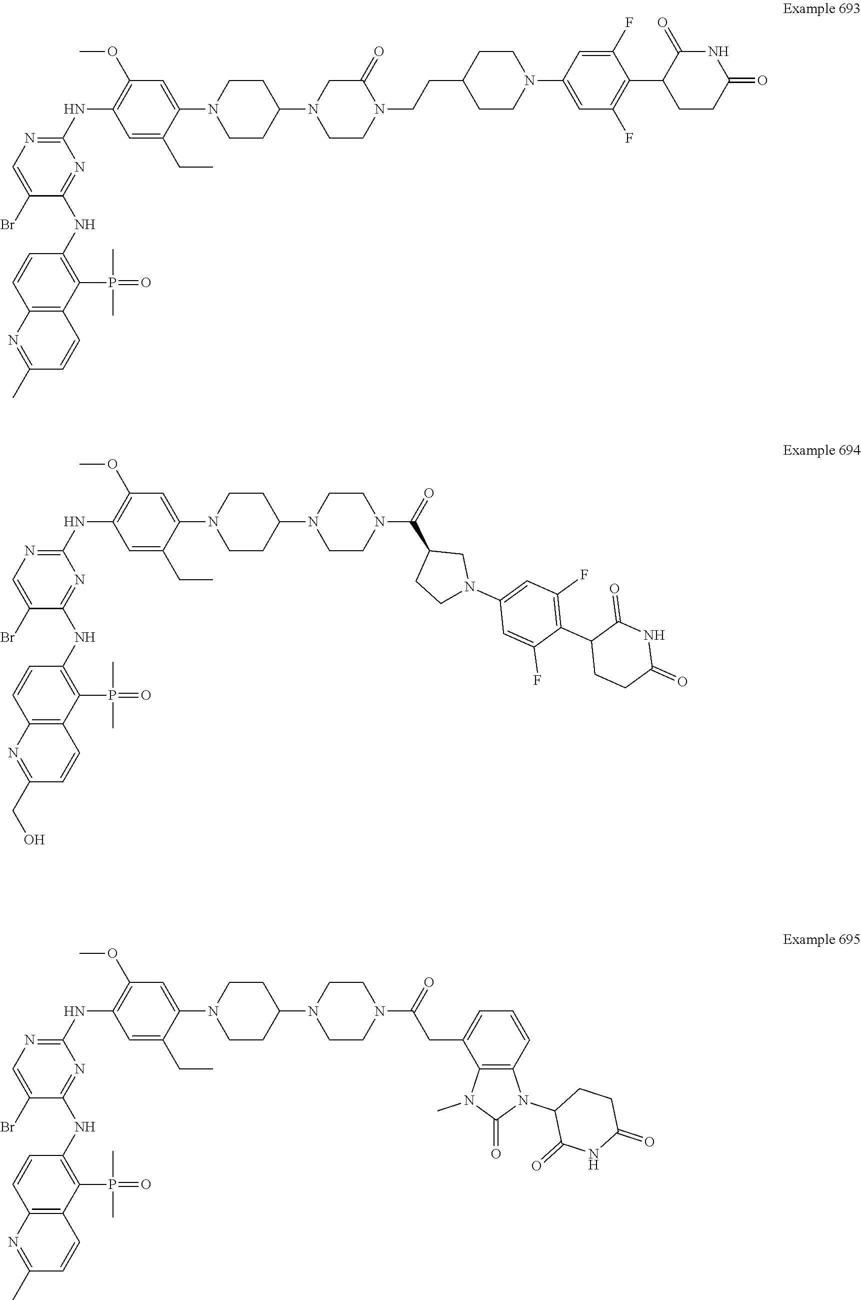 [SMILES: CCc1cc(Nc2ncc(Br)c(Nc3ccc4nc(C)ccc4c3P(C)(C)=O)n2)c(OC)cc1N1CCC(N2CCN(CCC3CCN(c4cc(F)c(C5CCC(=O)NC5=O)c(F)c4)CC3)C(=O)C2)CC1 ; CCc1cc(Nc2ncc(Br)c(Nc3ccc4nc(CO)ccc4c3P(C)(C)=O)n2)c(OC)cc1N1CCC(N2CCN(C(=O)[C@@H]3CCN(c4cc(F)c(C5CCC(=O)NC5=O)c(F)c4)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)Cc3cccc4c3n(C)c(=O)n4C3CCC(=O)NC3=O)CC2)CC1]

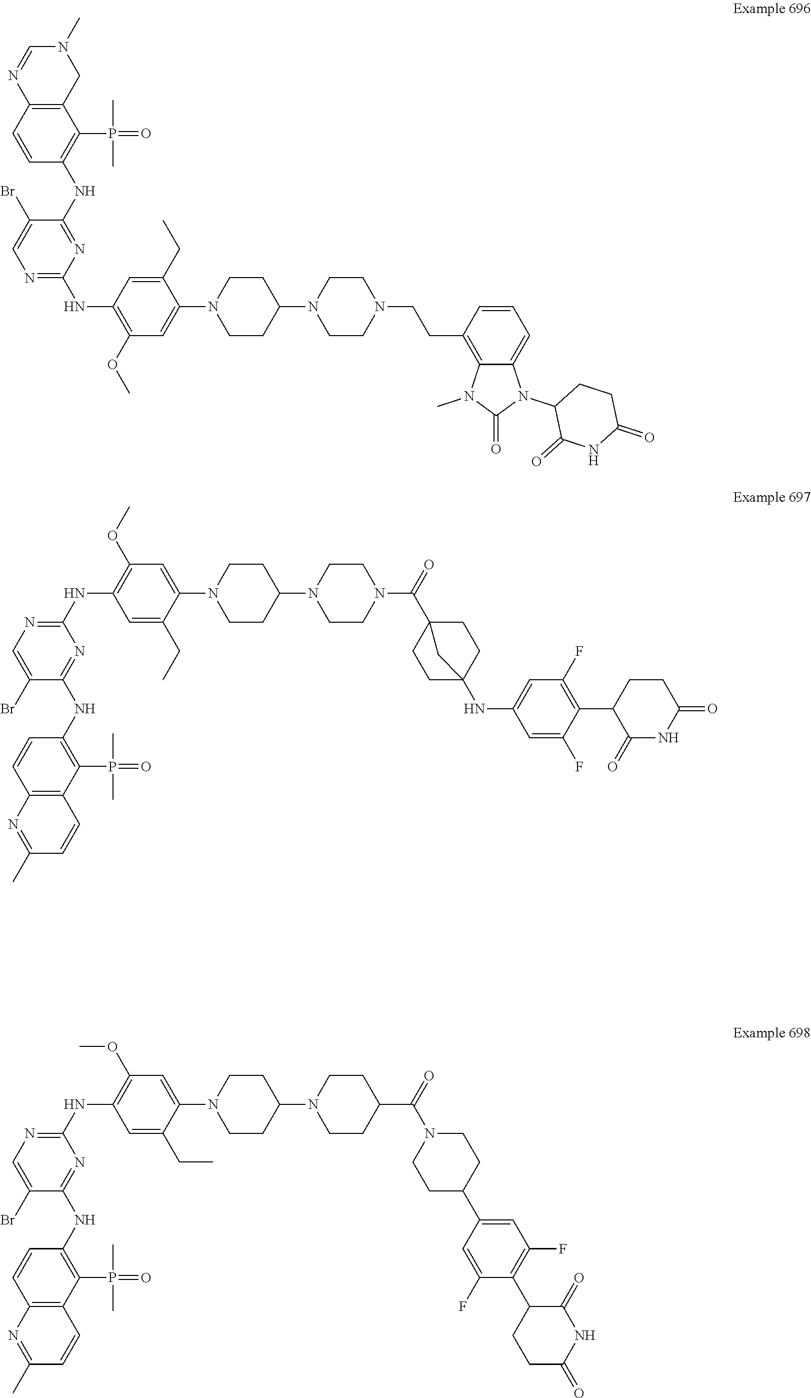 [SMILES: CCc1cc(Nc2ncc(Br)c(Nc3ccc4c(c3P(C)(C)=O)CN(C)C=N4)n2)c(OC)cc1N1CCC(N2CCN(CCc3cccc4c3n(C)c(=O)n4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(C(=O)C34CCC(Nc5cc(F)c(C6CCC(=O)NC6=O)c(F)c5)(CC3)C4)CC2)CC1 ; CCc1cc(Nc2ncc(Br)c(Nc3ccc4nc(C)ccc4c3P(C)(C)=O)n2)c(OC)cc1N1CCC(N2CCC(C(=O)N3CCC(c4cc(F)c(C5CCC(=O)NC5=O)c(F)c4)CC3)CC2)CC1]

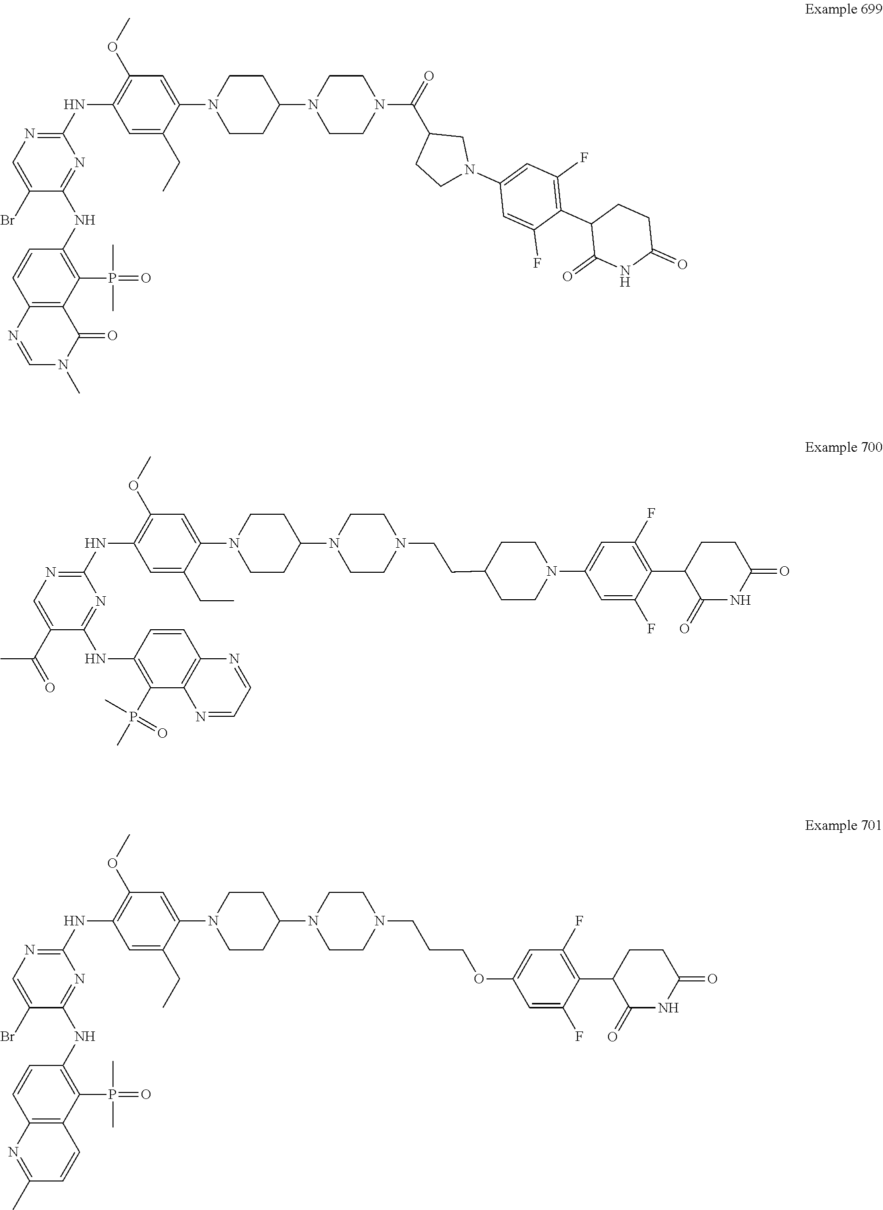 [SMILES: CCc1cc(Nc2ncc(Br)c(Nc3ccc4ncn(C)c(=O)c4c3P(C)(C)=O)n2)c(OC)cc1N1CCC(N2CCN(C(=O)C3CCN(c4cc(F)c(C5CCC(=O)NC5=O)c(F)c4)C3)CC2)CC1 ; CCc1cc(Nc2ncc(C(C)=O)c(Nc3ccc4nccnc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4nc(C)ccc4c3P(C)(C)=O)n2)c(OC)cc1N1CCC(N2CCN(CCCOc3cc(F)c(C4CCC(=O)NC4=O)c(F)c3)CC2)CC1]

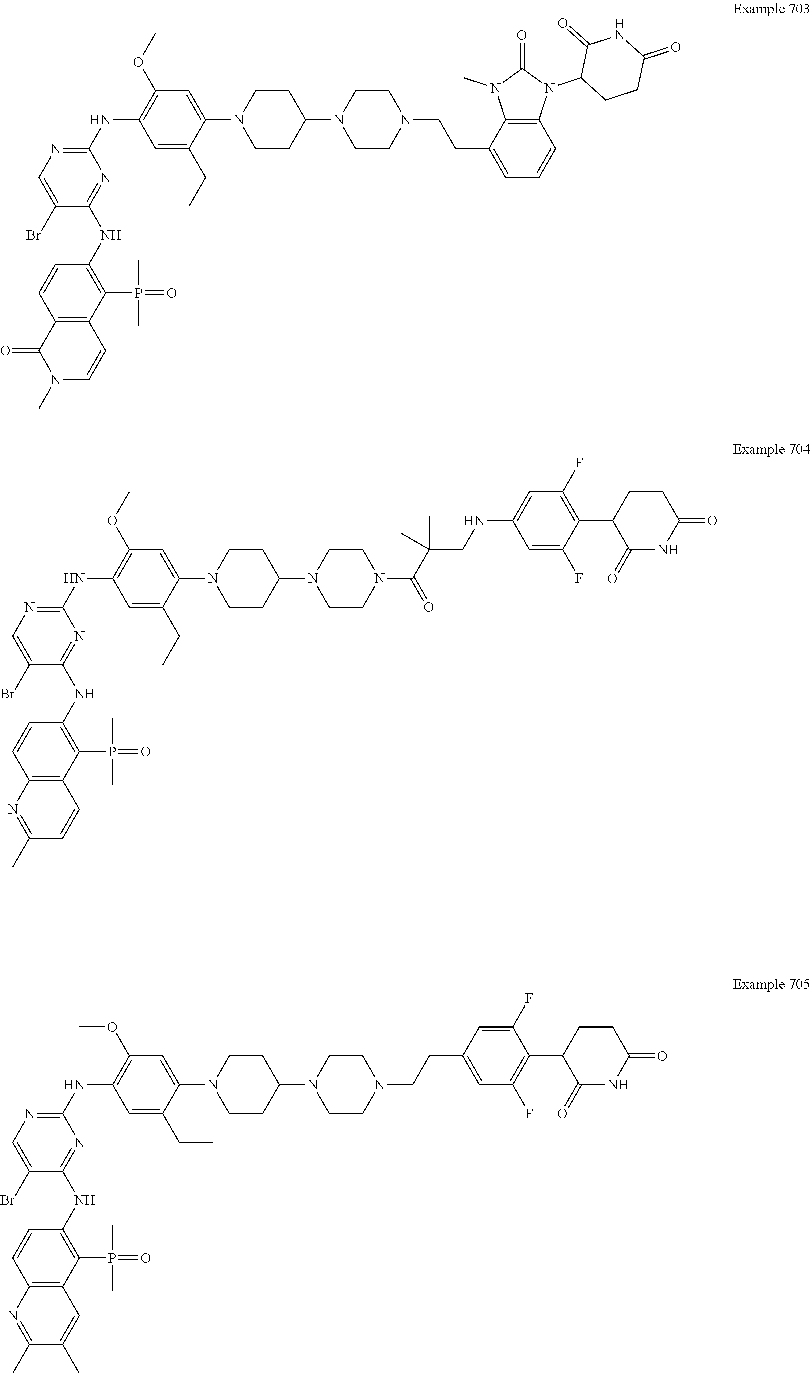 [SMILES: CCc1cc(Nc2ncc(Br)c(Nc3ccc4c(=O)n(C)ccc4c3P(C)(C)=O)n2)c(OC)cc1N1CCC(N2CCN(CCc3cccc4c3n(C)c(=O)n4C3CCC(=O)NC3=O)CC2)CC1 ; CCc1cc(Nc2ncc(Br)c(Nc3ccc4nc(C)ccc4c3P(C)(C)=O)n2)c(OC)cc1N1CCC(N2CCN(C(=O)C(C)(C)CNc3cc(F)c(C4CCC(=O)NC4=O)c(F)c3)CC2)CC1 ; CCc1cc(Nc2ncc(Br)c(Nc3ccc4nc(C)c(C)cc4c3P(C)(C)=O)n2)c(OC)cc1N1CCC(N2CCN(CCc3cc(F)c(C4CCC(=O)NC4=O)c(F)c3)CC2)CC1]

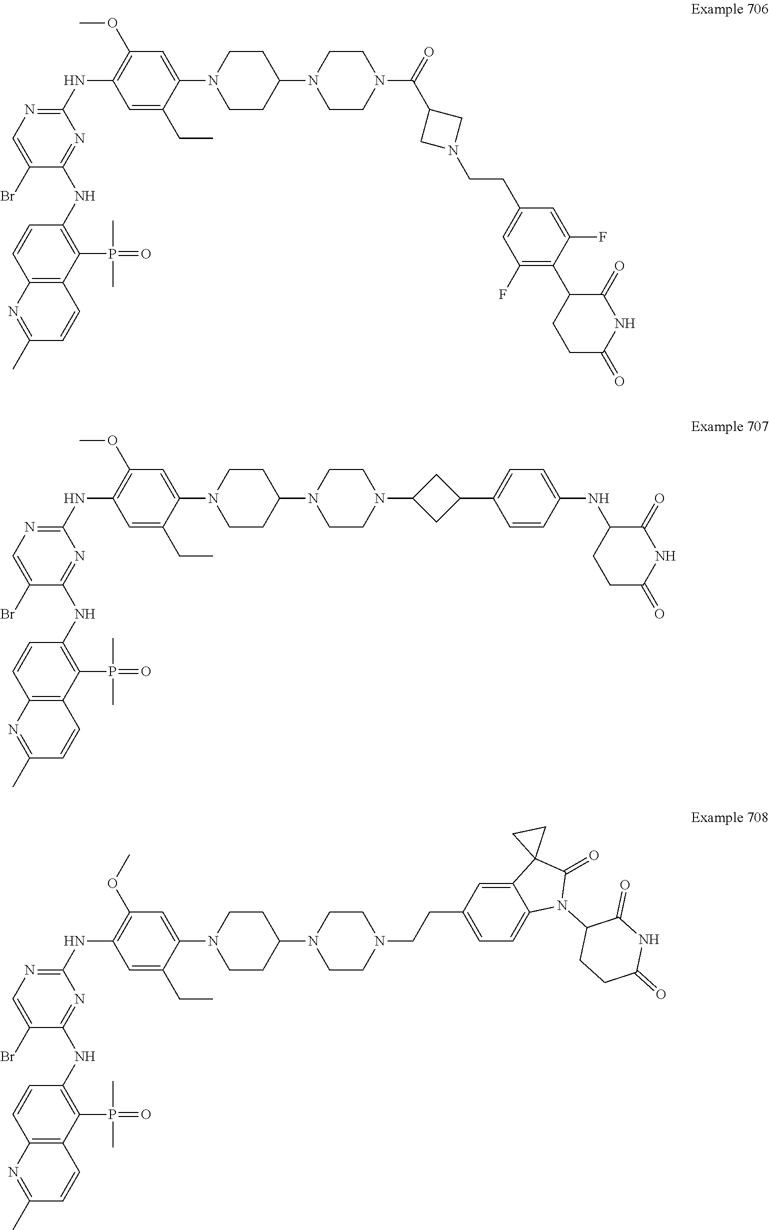 [SMILES: CCc1cc(Nc2ncc(Br)c(Nc3ccc4nc(C)ccc4c3P(C)(C)=O)n2)c(OC)cc1N1CCC(N2CCN(C(=O)C3CN(CCc4cc(F)c(C5CCC(=O)NC5=O)c(F)c4)C3)CC2)CC1 ; CCc1cc(Nc2ncc(Br)c(Nc3ccc4nc(C)ccc4c3P(C)(C)=O)n2)c(OC)cc1N1CCC(N2CCN(C3CC(c4ccc(NC5CCC(=O)NC5=O)cc4)C3)CC2)CC1 ; CCc1cc(Nc2ncc(Br)c(Nc3ccc4nc(C)ccc4c3P(C)(C)=O)n2)c(OC)cc1N1CCC(N2CCN(CCc3ccc4c(c3)C3(CC3)C(=O)N4C3CCC(=O)NC3=O)CC2)CC1]

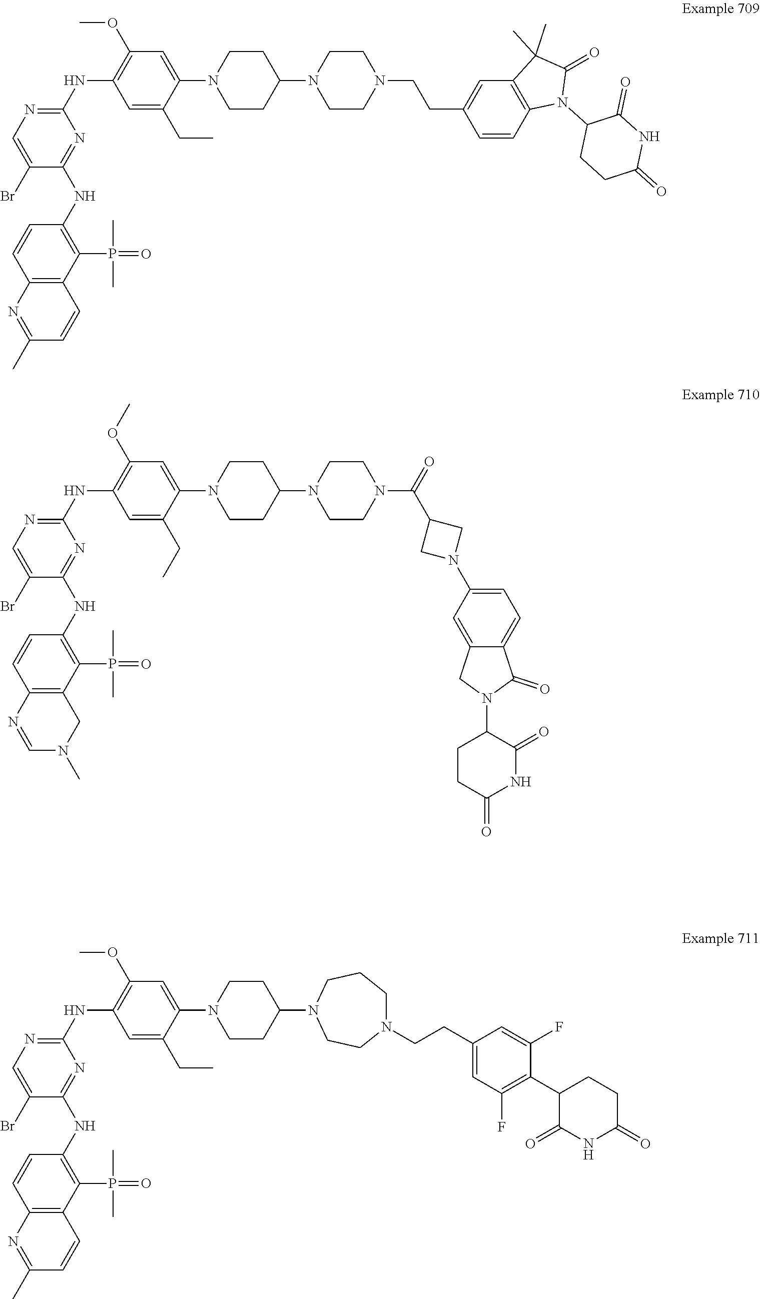 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)C(C)(C)C(=O)N4C3CCC(=O)NC3=O)CC2)CC1 ; CCc1cc(Nc2ncc(Br)c(Nc3ccc4c(c3P(C)(C)=O)CN(C)C=N4)n2)c(OC)cc1N1CCC(N2CCN(C(=O)C3CN(c4ccc5c(c4)CN(C4CCC(=O)NC4=O)C5=O)C3)CC2)CC1 ; CCc1cc(Nc2ncc(Br)c(Nc3ccc4nc(C)ccc4c3P(C)(C)=O)n2)c(OC)cc1N1CCC(N2CCCN(CCc3cc(F)c(C4CCC(=O)NC4=O)c(F)c3)CC2)CC1]

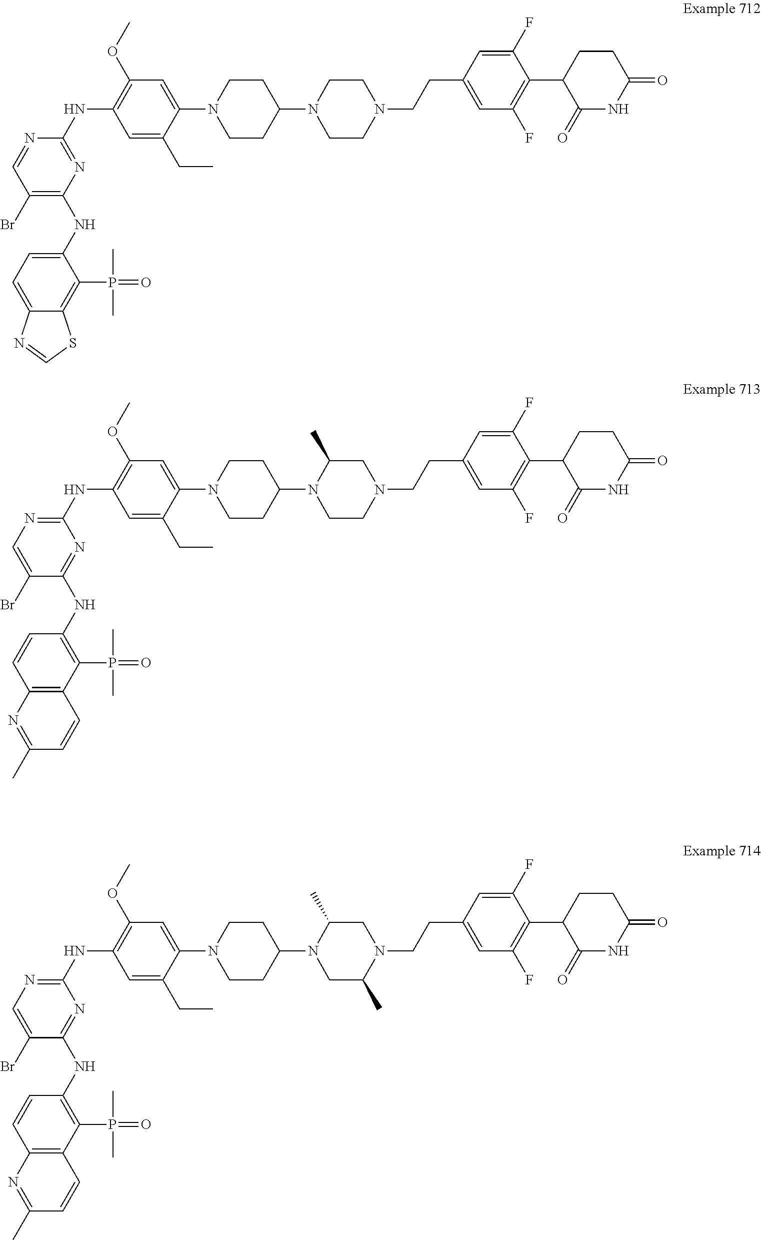 [SMILES: CCc1cc(Nc2ncc(Br)c(Nc3ccc4ncsc4c3P(C)(C)=O)n2)c(OC)cc1N1CCC(N2CCN(CCc3cc(F)c(C4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CCN(CCc3cc(F)c(C4CCC(=O)NC4=O)c(F)c3)C[C@@H]2C)CC1 ; CCc1cc(Nc2ncc(Br)c(Nc3ccc4nc(C)ccc4c3P(C)(C)=O)n2)c(OC)cc1N1CCC(N2C[C@H](C)N(CCc3cc(F)c(C4CCC(=O)NC4=O)c(F)c3)C[C@H]2C)CC1]

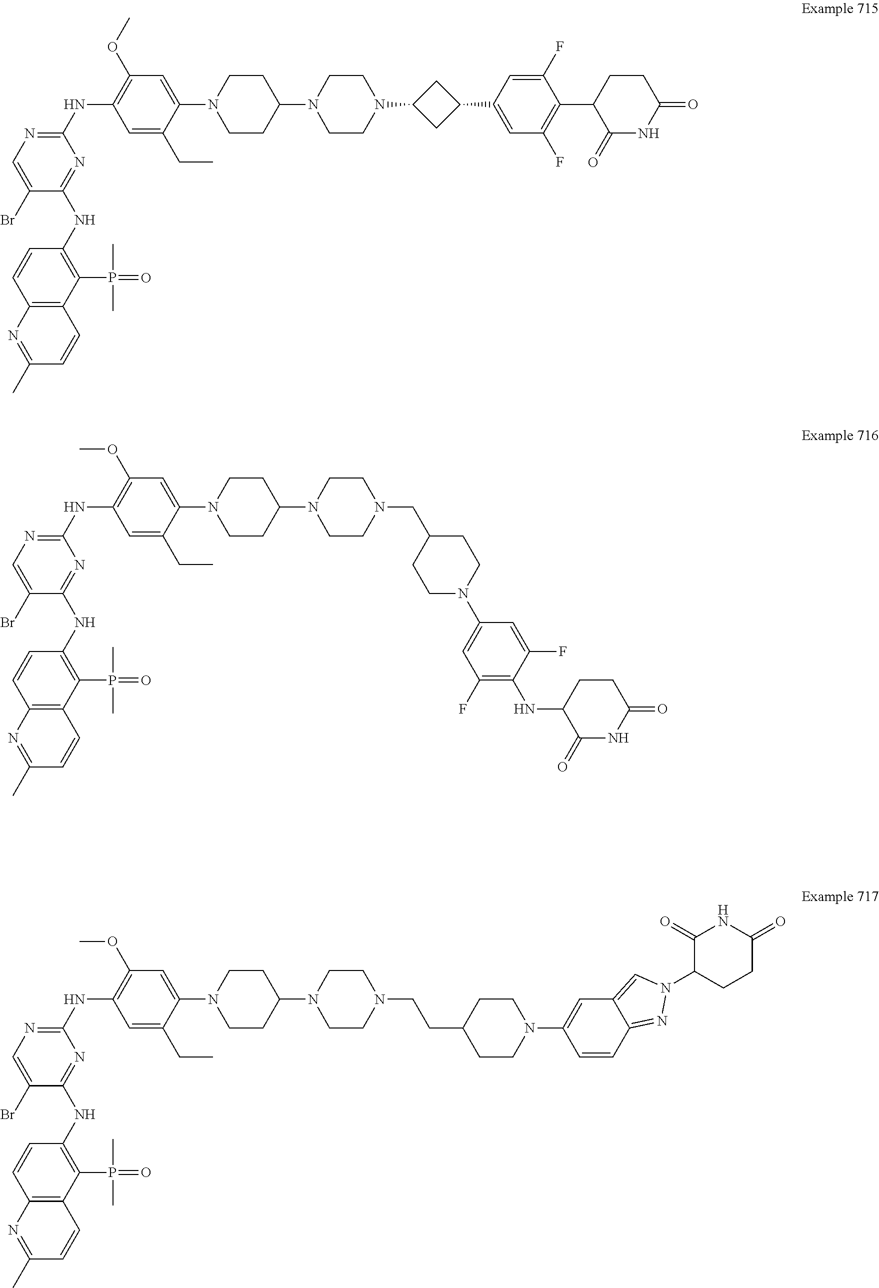 [SMILES: CCc1cc(Nc2ncc(Br)c(Nc3ccc4nc(C)ccc4c3P(C)(C)=O)n2)c(OC)cc1N1CCC(N2CCN([C@H]3C[C@@H](c4cc(F)c(C5CCC(=O)NC5=O)c(F)c4)C3)CC2)CC1 ; CCc1cc(Nc2ncc(Br)c(Nc3ccc4nc(C)ccc4c3P(C)(C)=O)n2)c(OC)cc1N1CCC(N2CCN(CC3CCN(c4cc(F)c(NC5CCC(=O)NC5=O)c(F)c4)CC3)CC2)CC1 ; CCc1cc(Nc2ncc(Br)c(Nc3ccc4nc(C)ccc4c3P(C)(C)=O)n2)c(OC)cc1N1CCC(N2CCN(CCC3CCN(c4ccc5nn(C6CCC(=O)NC6=O)cc5c4)CC3)CC2)CC1]

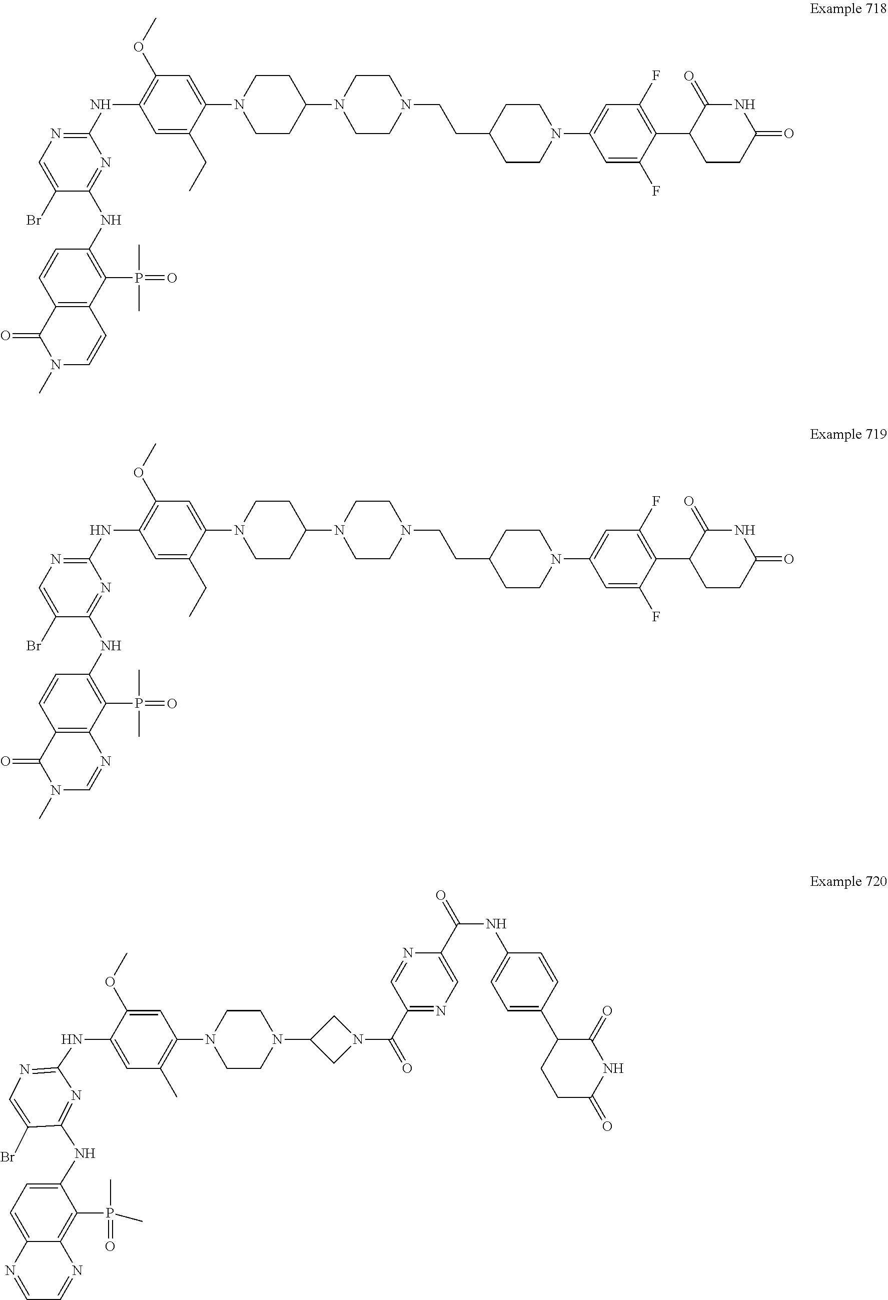 [SMILES: CCc1cc(Nc2ncc(Br)c(Nc3ccc4c(=O)n(C)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; CCc1cc(Nc2ncc(Br)c(Nc3ccc4c(=O)n(C)cnc4c3P(C)(C)=O)n2)c(OC)cc1N1CCC(N2CCN(CCC3CCN(c4cc(F)c(C5CCC(=O)NC5=O)c(F)c4)CC3)CC2)CC1 ; COc1cc(N2CCN(C3CN(C(=O)c4cnc(C(=O)Nc5ccc(C6CCC(=O)NC6=O)cc5)cn4)C3)CC2)c(C)cc1Nc1ncc(Br)c(Nc2ccc3nccnc3c2P(C)(C)=O)n1]

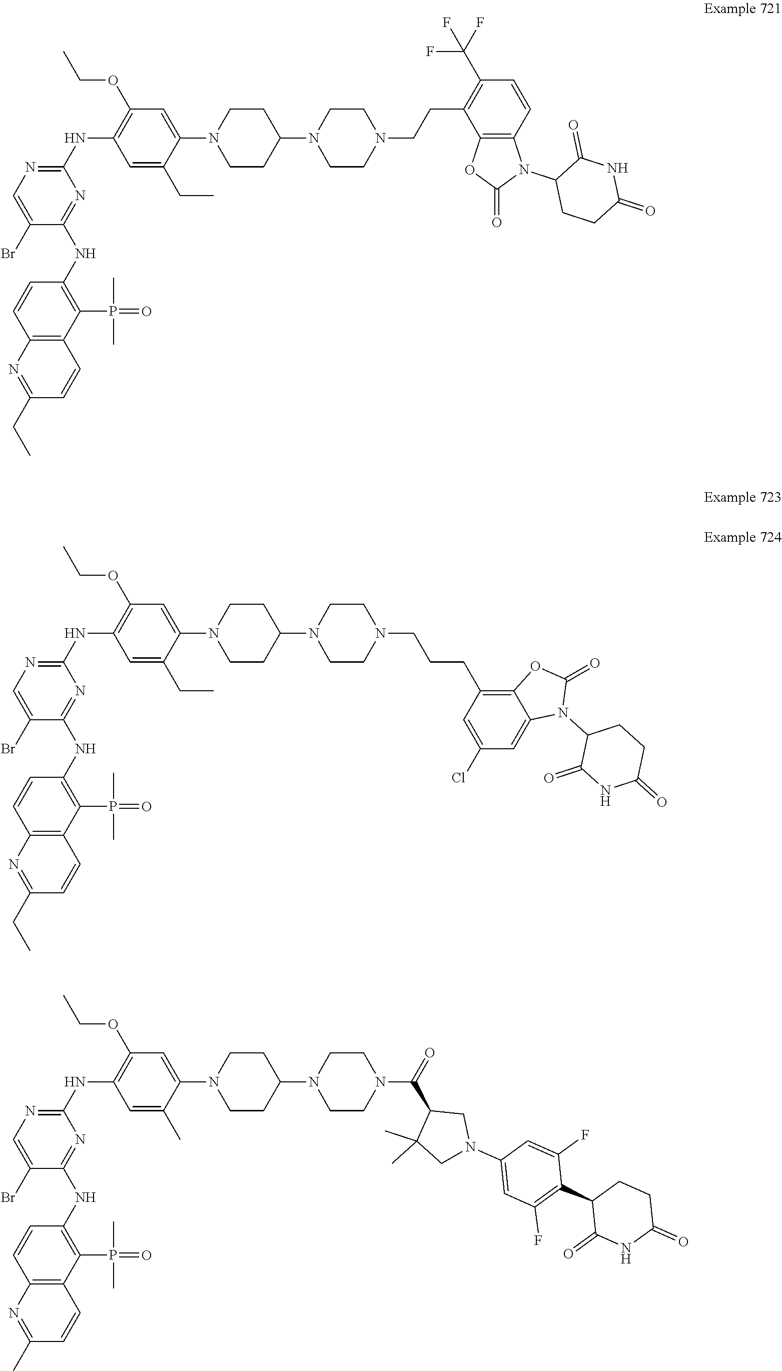 [SMILES: CCOc1cc(N2CCC(N3CCN(CCc4c(C(F)(F)F)ccc5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(CCCc4cc(Cl)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; CCOc1cc(N2CCC(N3CCN(C(=O)[C@H]4CN(c5cc(F)c([C@H]6CCC(=O)NC6=O)c(F)c5)CC4(C)C)CC3)CC2)c(C)cc1Nc1ncc(Br)c(Nc2ccc3nc(C)ccc3c2P(C)(C)=O)n1]

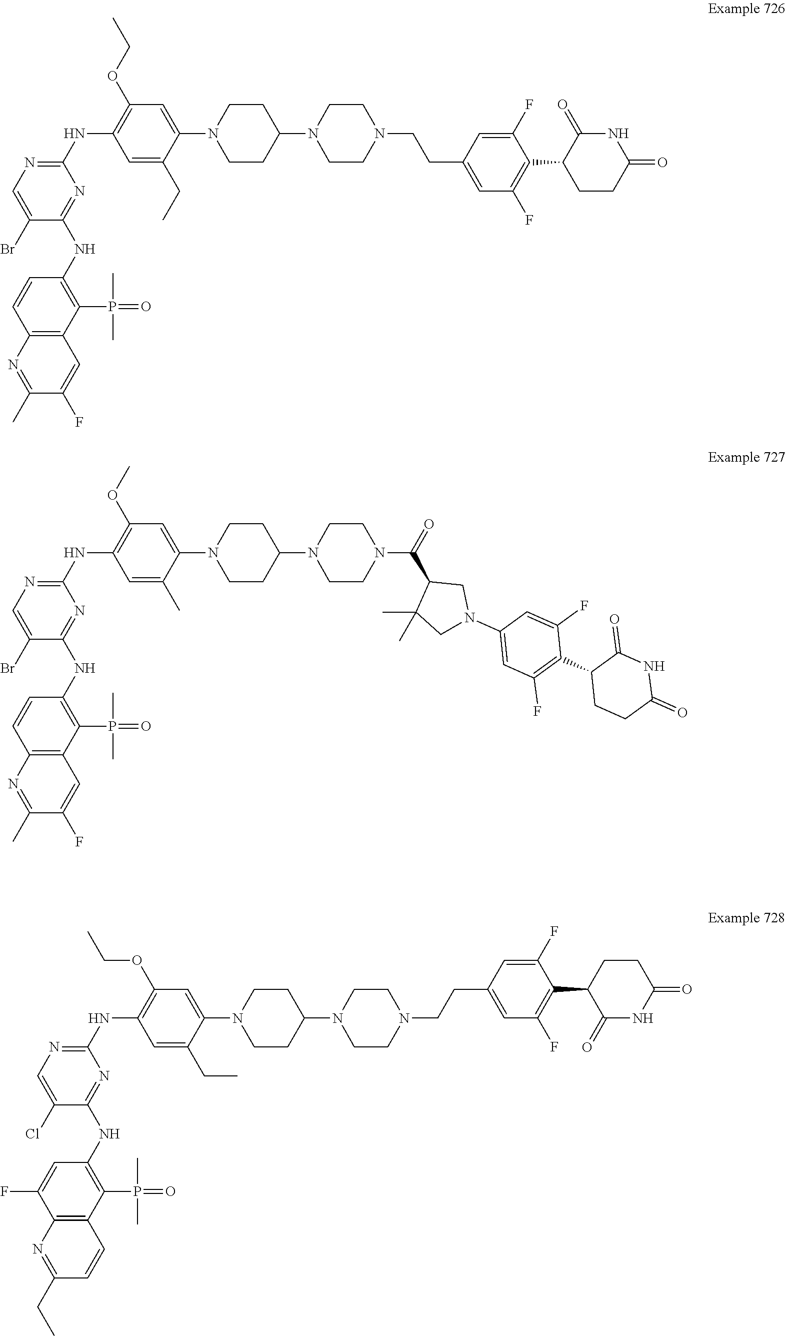 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(C)c(F)cc3c2P(C)(C)=O)n1 ; COc1cc(N2CCC(N3CCN(C(=O)[C@H]4CN(c5cc(F)c([C@H]6CCC(=O)NC6=O)c(F)c5)CC4(C)C)CC3)CC2)c(C)cc1Nc1ncc(Br)c(Nc2ccc3nc(C)c(F)cc3c2P(C)(C)=O)n1 ; CCOc1cc(N2CCC(N3CCN(CCc4cc(F)c([C@H]5CCC(=O)NC5=O)c(F)c4)CC3)CC2)c(CC)cc1Nc1ncc(Cl)c(Nc2cc(F)c3nc(CC)ccc3c2P(C)(C)=O)n1]

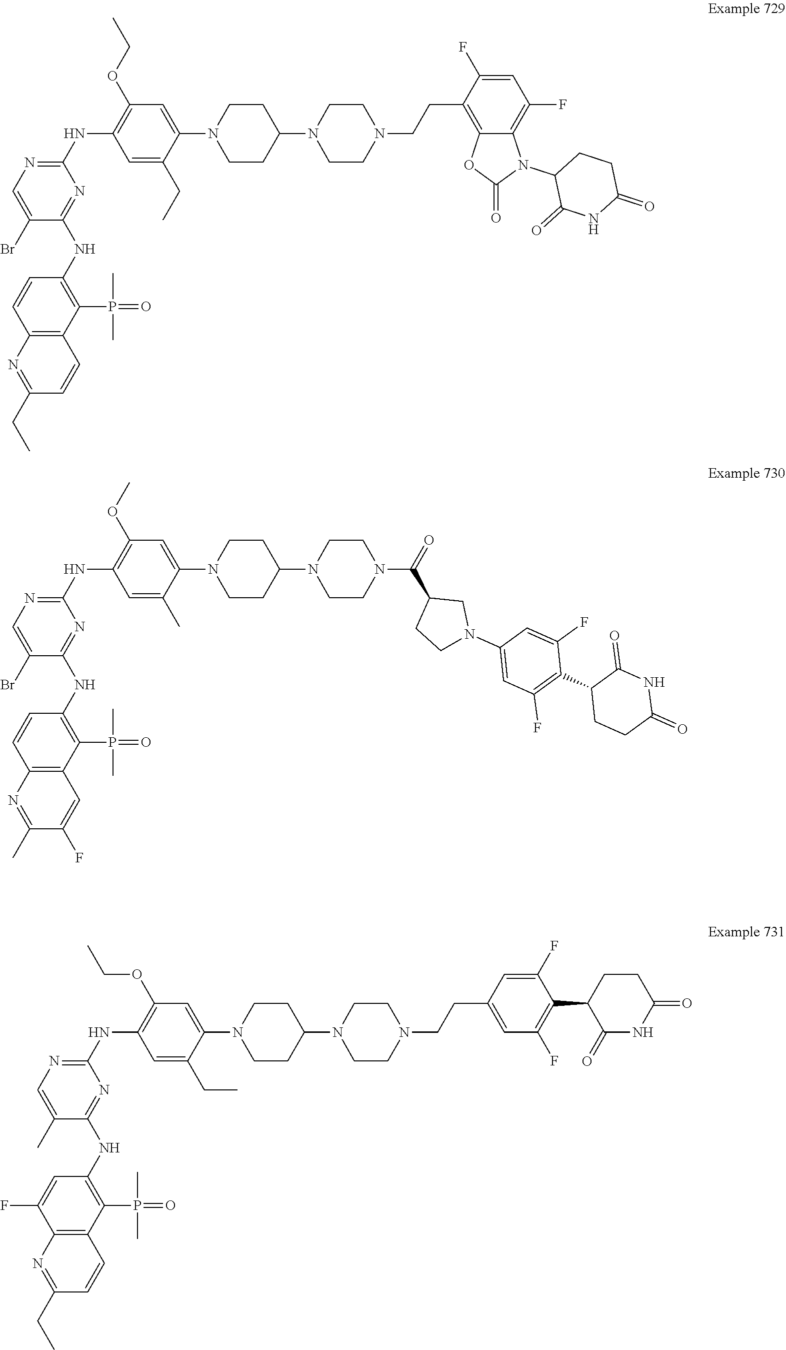 [SMILES: CCOc1cc(N2CCC(N3CCN(CCc4c(F)cc(F)c5c4oc(=O)n5C4CCC(=O)NC4=O)CC3)CC2)c(CC)cc1Nc1ncc(Br)c(Nc2ccc3nc(CC)ccc3c2P(C)(C)=O)n1 ; COc1cc(N2CCC(N3CCN(C(=O)[C@@H]4CCN(c5cc(F)c([C@H]6CCC(=O)NC6=O)c(F)c5)C4)CC3)CC2)c(C)cc1Nc1ncc(Br)c(Nc2ccc3nc(C)c(F)cc3c2P(C)(C)=O)n1 ; CCOc1cc(N2CCC(N3CCN(CCc4cc(F)c([C@H]5CCC(=O)NC5=O)c(F)c4)CC3)CC2)c(CC)cc1Nc1ncc(C)c(Nc2cc(F)c3nc(CC)ccc3c2P(C)(C)=O)n1]

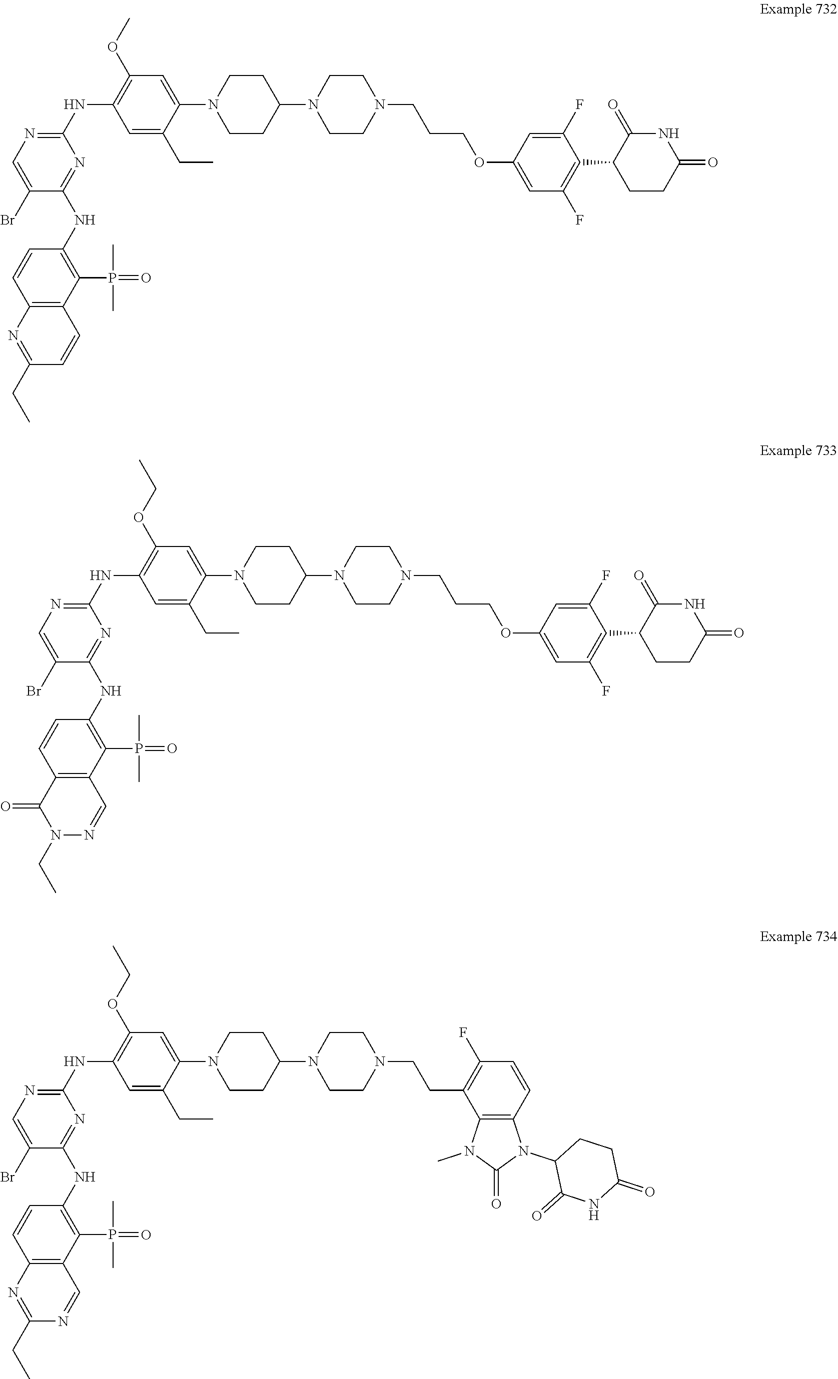 [SMILES: CCc1ccc2c(P(C)(C)=O)c(Nc3nc(Nc4cc(CC)c(N5CCC(N6CCN(CCCOc7cc(F)c([C@H]8CCC(=O)NC8=O)c(F)c7)CC6)CC5)cc4OC)ncc3Br)ccc2n1 ; CCOc1cc(N2CCC(N3CCN(CCCOc4cc(F)c([C@H]5CCC(=O)NC5=O)c(F)c4)CC3)CC2)c(CC)cc1Nc1ncc(Br)c(Nc2ccc3c(=O)n(CC)ncc3c2P(C)(C)=O)n1 ; CCOc1cc(N2CCC(N3CCN(CCc4c(F)ccc5c4n(C)c(=O)n5C4CCC(=O)NC4=O)CC3)CC2)c(CC)cc1Nc1ncc(Br)c(Nc2ccc3nc(CC)ncc3c2P(C)(C)=O)n1]

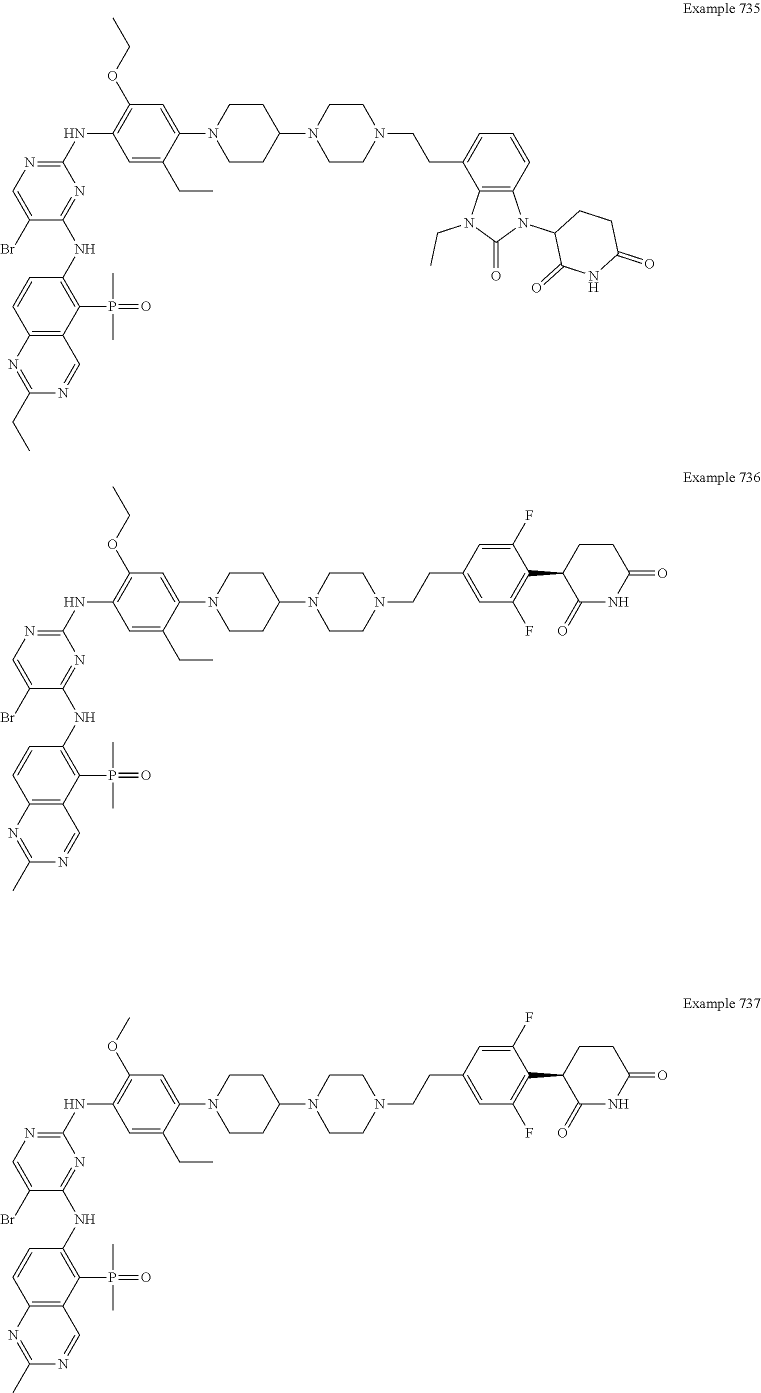 [SMILES: CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2ccc3nc(CC)ncc3c2P(C)(C)=O)n1 ; CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(C)ncc3c2P(C)(C)=O)n1 ; CCc1cc(Nc2ncc(Br)c(Nc3ccc4nc(C)ncc4c3P(C)(C)=O)n2)c(OC)cc1N1CCC(N2CCN(CCc3cc(F)c([C@H]4CCC(=O)NC4=O)c(F)c3)CC2)CC1]

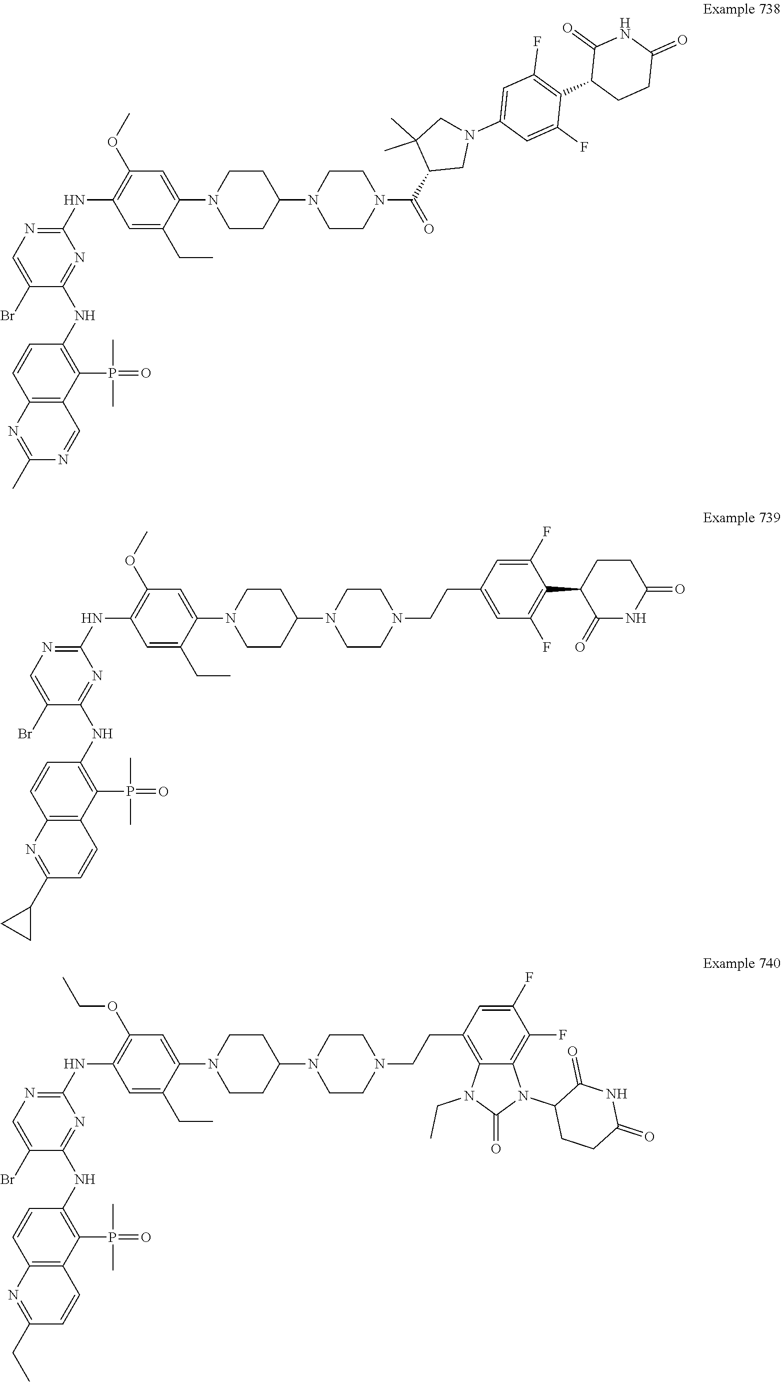 [SMILES: CCc1cc(Nc2ncc(Br)c(Nc3ccc4nc(C)ncc4c3P(C)(C)=O)n2)c(OC)cc1N1CCC(N2CCN(C(=O)[C@H]3CN(c4cc(F)c([C@H]5CCC(=O)NC5=O)c(F)c4)CC3(C)C)CC2)CC1 ; CCc1cc(Nc2ncc(Br)c(Nc3ccc4nc(C5CC5)ccc4c3P(C)(C)=O)n2)c(OC)cc1N1CCC(N2CCN(CCc3cc(F)c([C@H]4CCC(=O)NC4=O)c(F)c3)CC2)CC1 ; CCOc1cc(N2CCC(N3CCN(CCc4cc(F)c(F)c5c4n(CC)c(=O)n5C4CCC(=O)NC4=O)CC3)CC2)c(CC)cc1Nc1ncc(Br)c(Nc2ccc3nc(CC)ccc3c2P(C)(C)=O)n1]

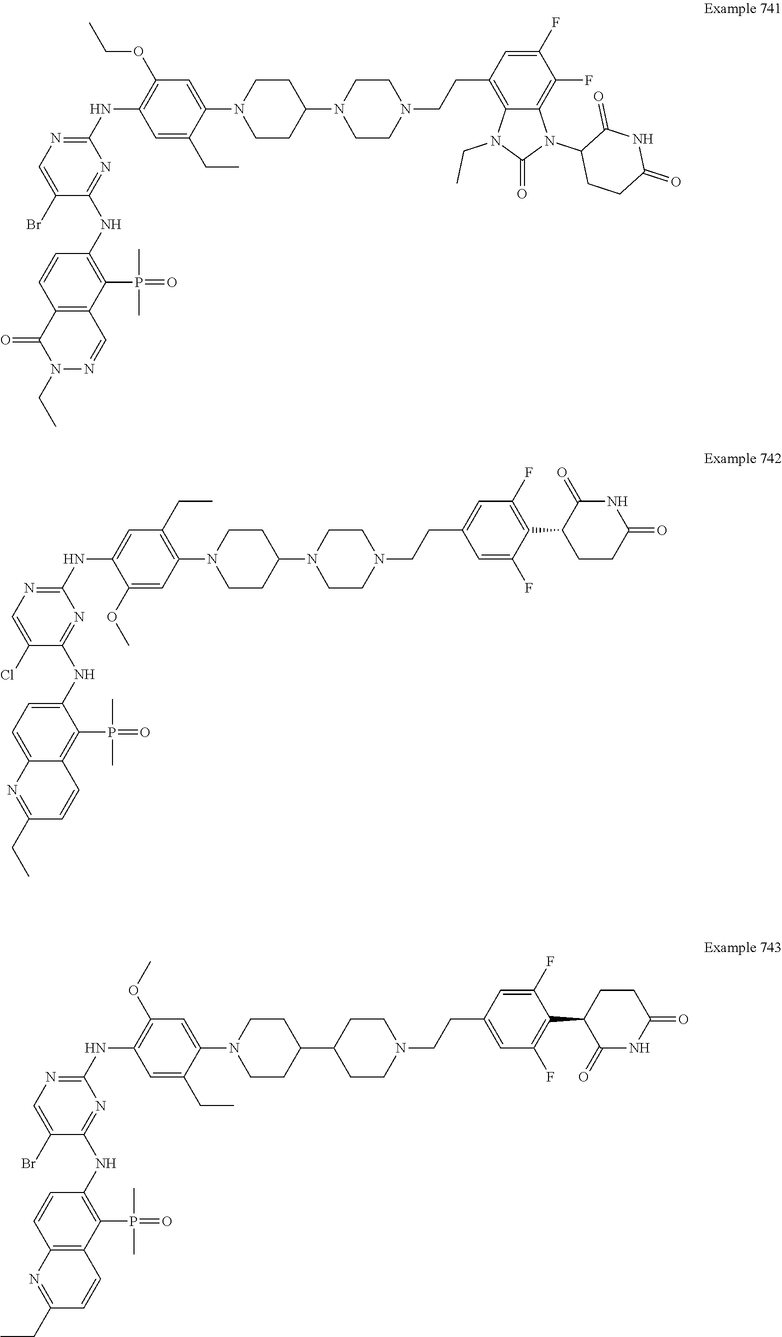 [SMILES: CCOc1cc(N2CCC(N3CCN(CCc4cc(F)c(F)c5c4n(CC)c(=O)n5C4CCC(=O)NC4=O)CC3)CC2)c(CC)cc1Nc1ncc(Br)c(Nc2ccc3c(=O)n(CC)ncc3c2P(C)(C)=O)n1 ; CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC)ncc3Cl)ccc2n1 ; CCc1ccc2c(P(C)(C)=O)c(Nc3nc(Nc4cc(CC)c(N5CCC(C6CCN(CCc7cc(F)c([C@H]8CCC(=O)NC8=O)c(F)c7)CC6)CC5)cc4OC)ncc3Br)ccc2n1]

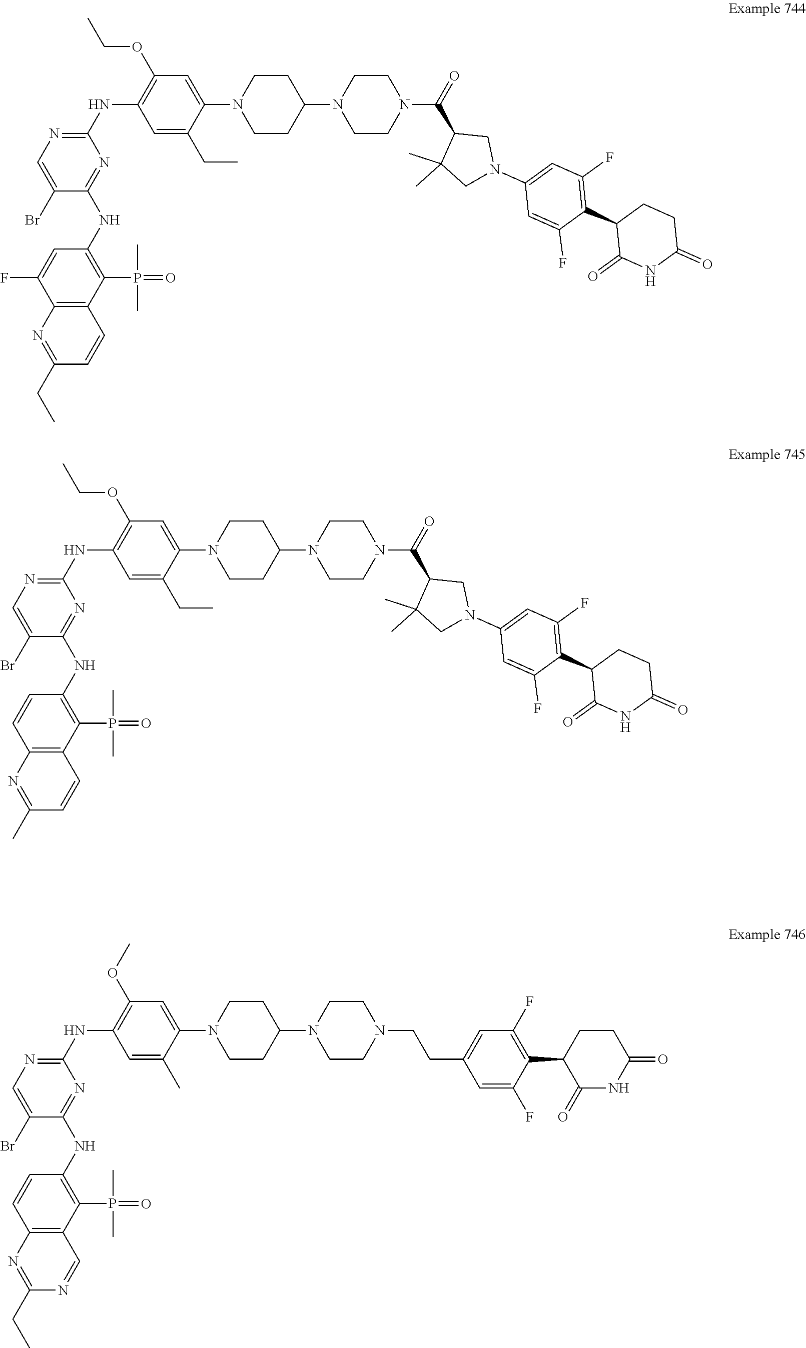

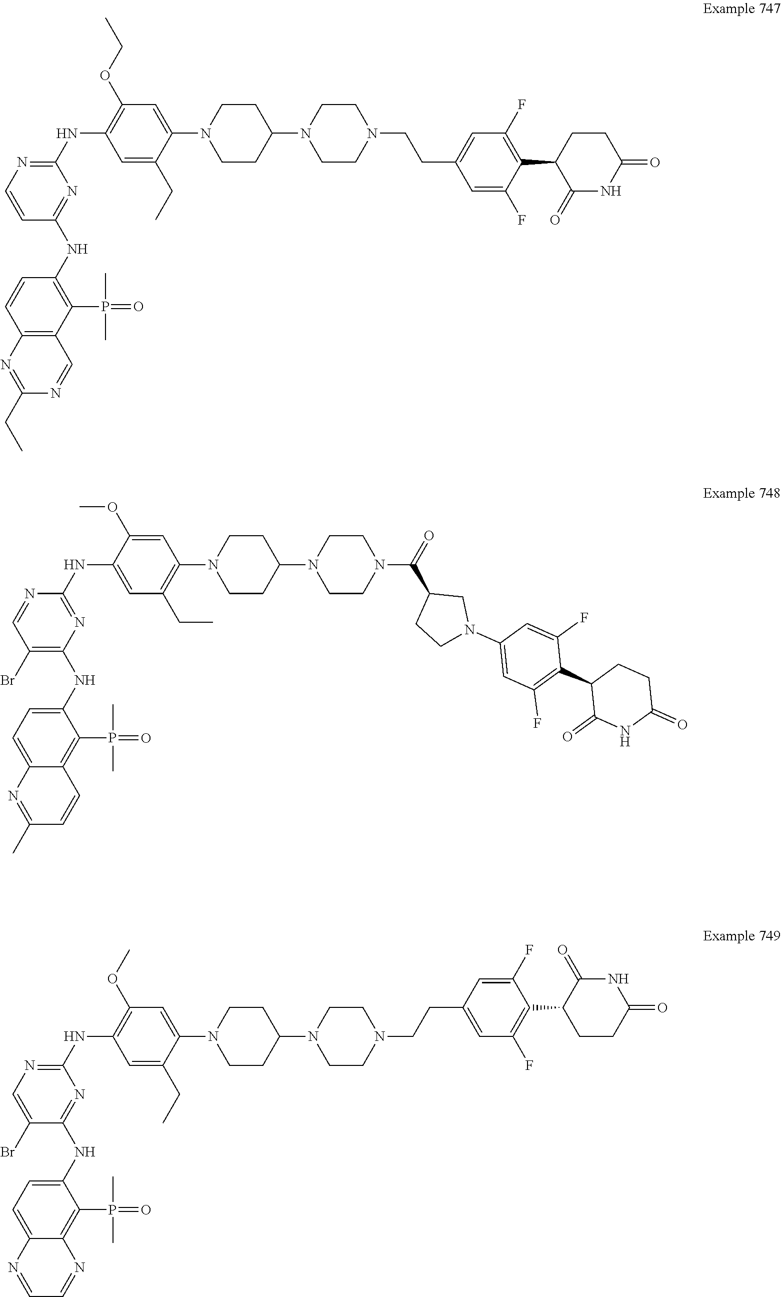 [SMILES: CCOc1cc(N2CCC(N3CCN(CCc4cc(F)c([C@H]5CCC(=O)NC5=O)c(F)c4)CC3)CC2)c(CC)cc1Nc1nccc(Nc2ccc3nc(CC)ncc3c2P(C)(C)=O)n1 ; CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1 ; CCc1cc(Nc2ncc(Br)c(Nc3ccc4nccnc4c3P(C)(C)=O)n2)c(OC)cc1N1CCC(N2CCN(CCc3cc(F)c([C@H]4CCC(=O)NC4=O)c(F)c3)CC2)CC1]

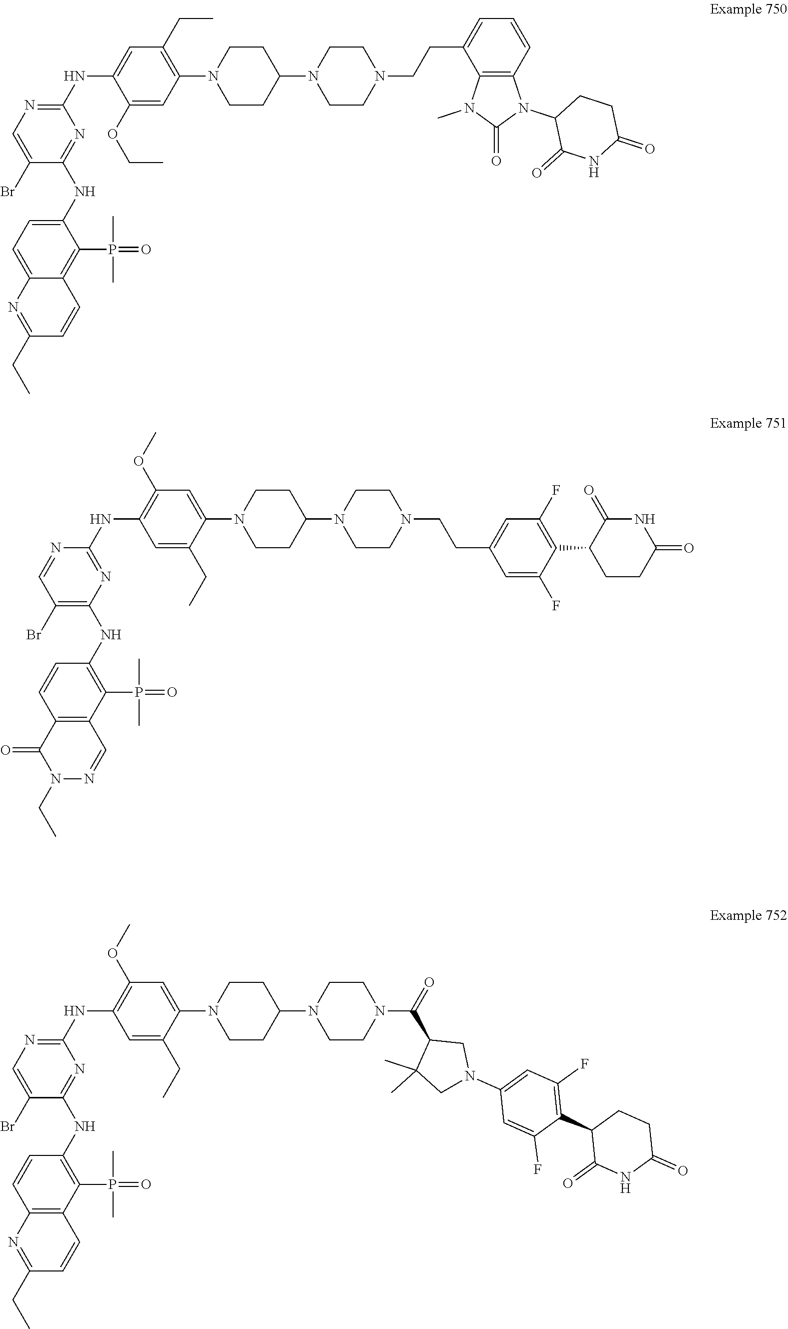 [SMILES: CCOc1cc(N2CCC(N3CCN(CCc4cccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1 ; CCc1cc(Nc2ncc(Br)c(Nc3ccc4c(=O)n(CC)ncc4c3P(C)(C)=O)n2)c(OC)cc1N1CCC(N2CCN(CCc3cc(F)c([C@H]4CCC(=O)NC4=O)c(F)c3)CC2)CC1 ; CCc1ccc2c(P(C)(C)=O)c(Nc3nc(Nc4cc(CC)c(N5CCC(N6CCN(C(=O)[C@H]7CN(c8cc(F)c([C@H]9CCC(=O)NC9=O)c(F)c8)CC7(C)C)CC6)CC5)cc4OC)ncc3Br)ccc2n1]

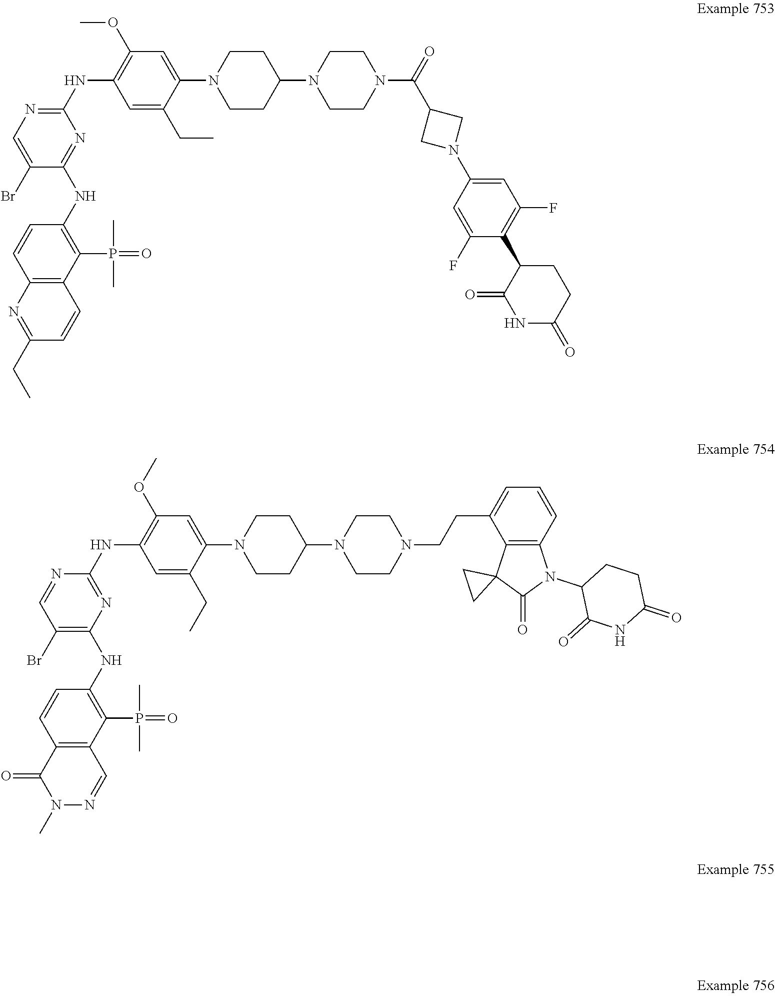 [SMILES: CCc1ccc2c(P(C)(C)=O)c(Nc3nc(Nc4cc(CC)c(N5CCC(N6CCN(C(=O)C7CN(c8cc(F)c([C@H]9CCC(=O)NC9=O)c(F)c8)C7)CC6)CC5)cc4OC)ncc3Br)ccc2n1 ; CCc1cc(Nc2ncc(Br)c(Nc3ccc4c(=O)n(C)ncc4c3P(C)(C)=O)n2)c(OC)cc1N1CCC(N2CCN(CCc3cccc4c3C3(CC3)C(=O)N4C3CCC(=O)NC3=O)CC2)CC1]

Aspect 50. A pharmaceutical composition comprising a compound of any one of Aspects 1-49 or a pharmaceutically acceptable salt, stereoisomer, tautomer or prodrug thereof, together with a pharmaceutically acceptable excipient.

Aspect 51. A method of treating a disease that can be affected by EGFR modulation, comprises administrating a subject in need thereof an effective amount of a compound of any one of Aspects 1-49 or a pharmaceutically acceptable salt, stereoisomer, tautomer or prodrug thereof.

Aspect 52. The method of Aspect 51, wherein the disease is selected from cancer, preferred pancreatic cancer, breast cancer, glioblastoma multiforme, head and neck cancer, or non-small cell lung cancer.

Aspect 53. Use of a compound of any one of Aspects 1-49 or a pharmaceutically acceptable salt, stereoisomer, tautomer or prodrug thereof in the preparation of a medicament for treating a disease that can be affected by EGFR modulation.

Aspect 54. The use of Aspect 53, wherein the disease is cancer, preferred pancreatic cancer, breast cancer, glioblastoma multiforme, head and neck cancer, or non-small cell lung cancer.

### DETAILED DESCRIPTION OF THE INVENTION

The following terms have the indicated meanings throughout the specification: Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended claims, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-1-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkynylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄0.6 cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring include, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term “H” or “hydrogen” disclosed herein includes Hydrogen and the non-radioisotope deuterium.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H, et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation. Prodrugs are frequently, although not necessarily, pharmacologically inactive until converted to the active agent.

“deuterated analog” refers to a derivative of an active agent that an arbitrary hydrogen is substituted with deuterium. In some embodiments, the deuterated site is on the Warhead moiety. In some embodiments, the deuterated site is on the Linker moiety. In some embodiments, the deuterated site is on the Degron moiety.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the claims which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the claims which follow, the term “Cₙ₋ₘ” indicates a range which includes the endpoints, wherein n and m are integers and indicate the number of carbons. Examples include C₁₋₈, C₁₋₆, and the like.

Unless specifically defined elsewhere in this document, all other technical and scientific terms used herein have the meaning commonly understood by one of ordinary skill in the art to which this invention belongs.

General Reaction Scheme for Compound Preparation

The subject compounds and pharmaceutically acceptable salts thereof, can be prepared from (a) commercially available starting materials (b) known starting materials which may be prepared as described in literature procedures (c) new intermediates described in the schemes and experimental procedures herein. In making the compounds of the invention, the order of synthetic steps may be varied to increase the yield of the desired product. Some of the compounds in this invention may be generated by the methods as shown in the following reaction schemes and the description thereof.

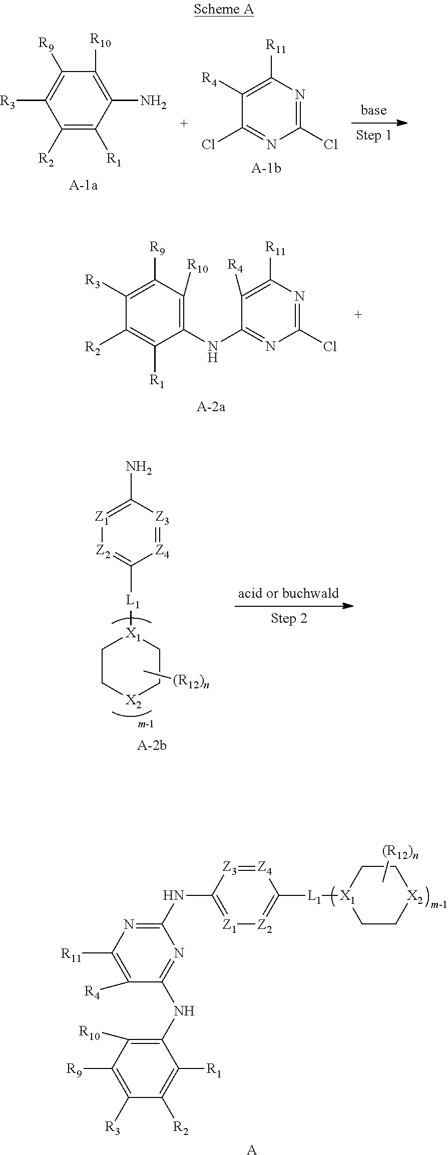

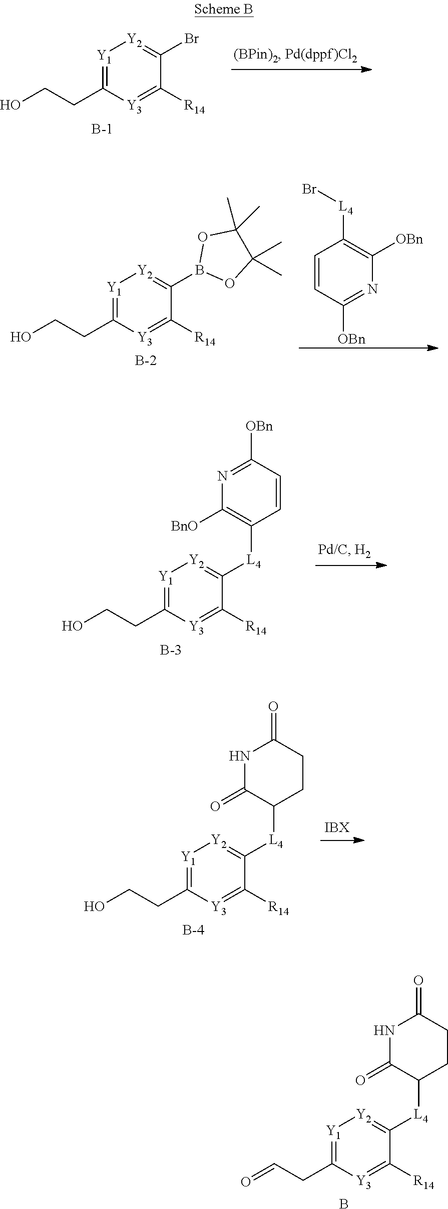

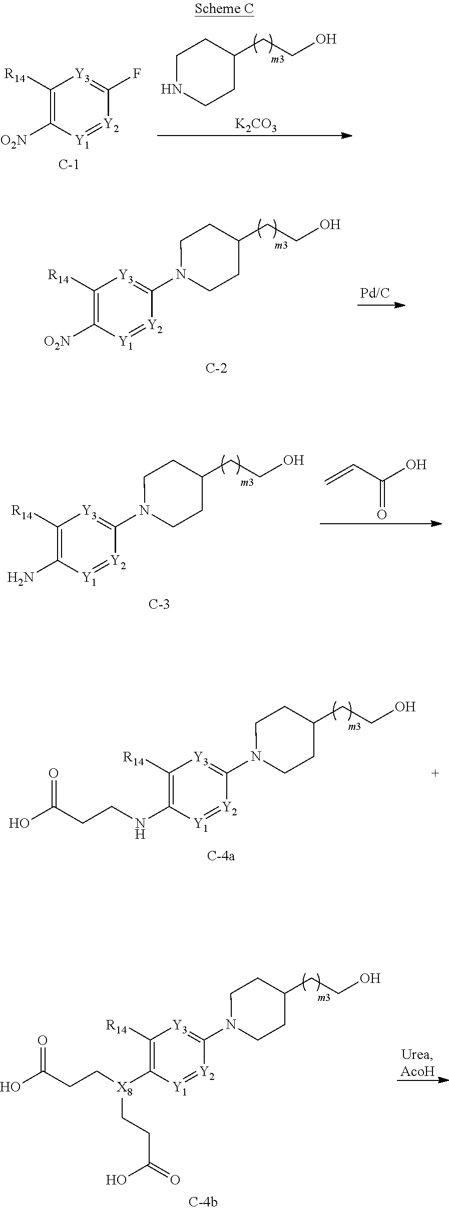

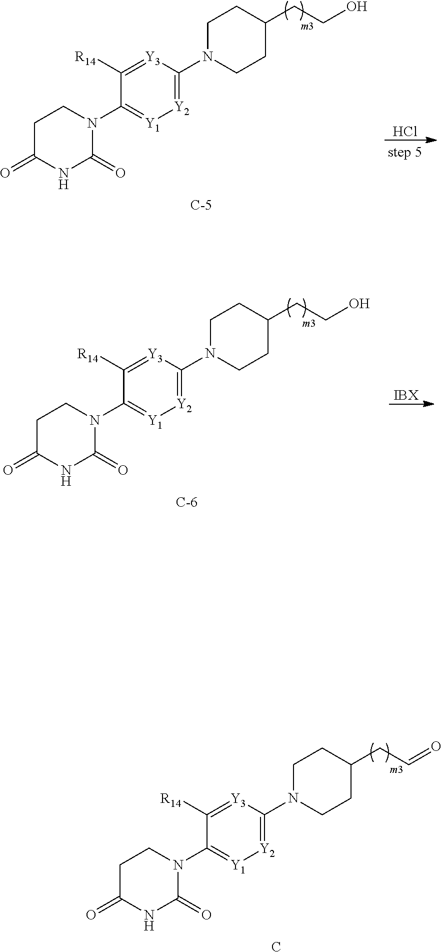

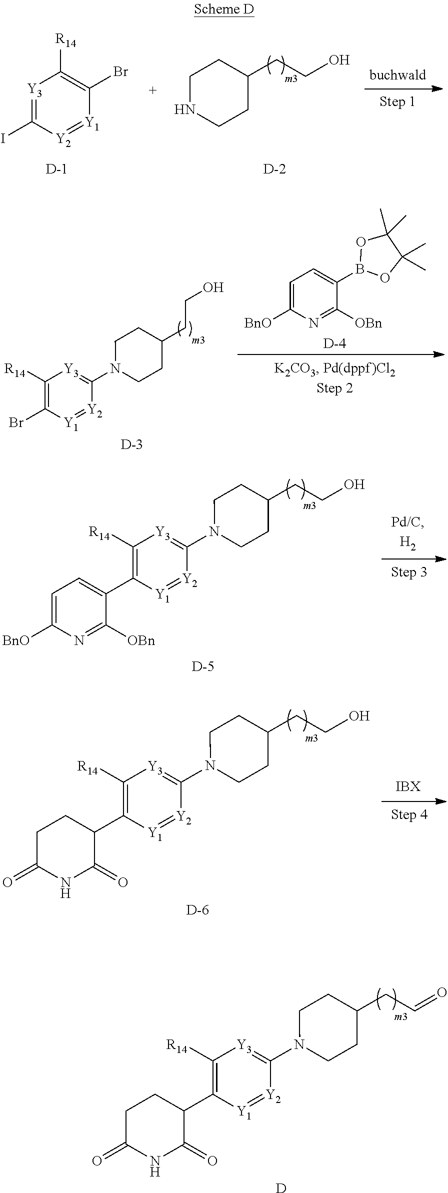

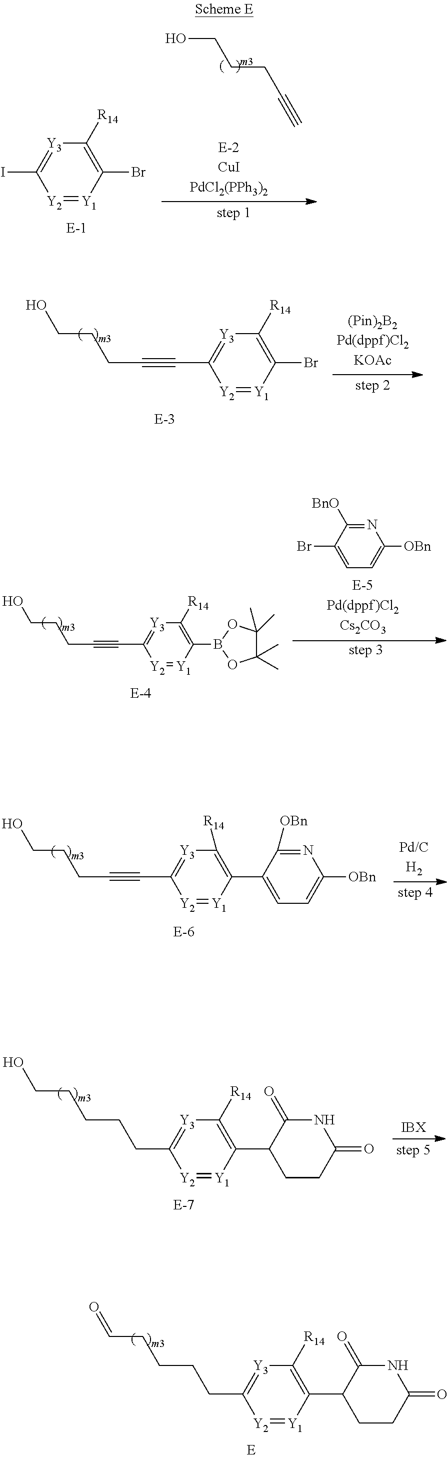

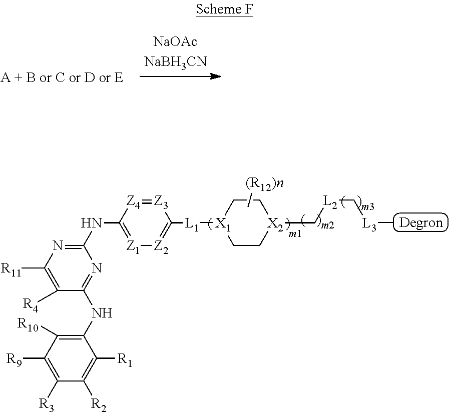

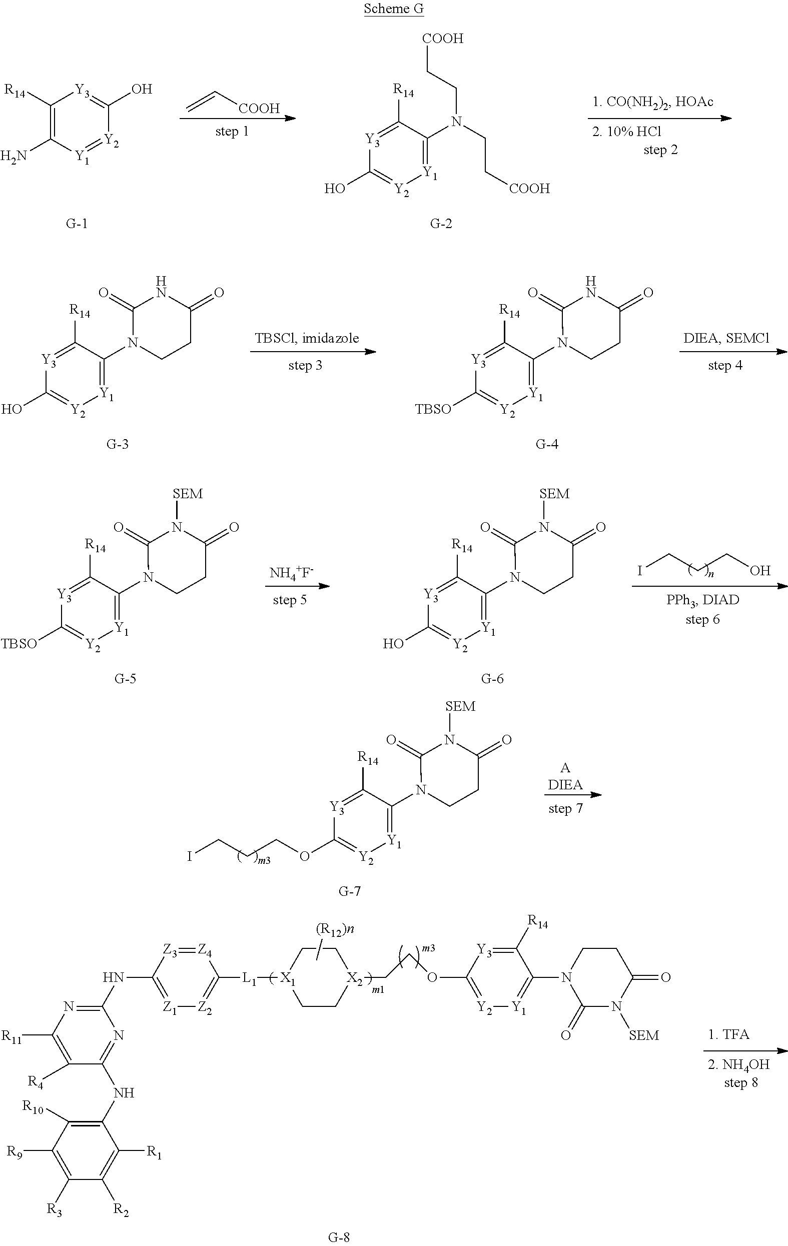

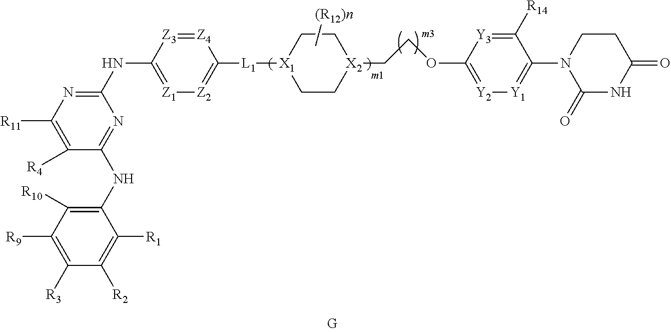

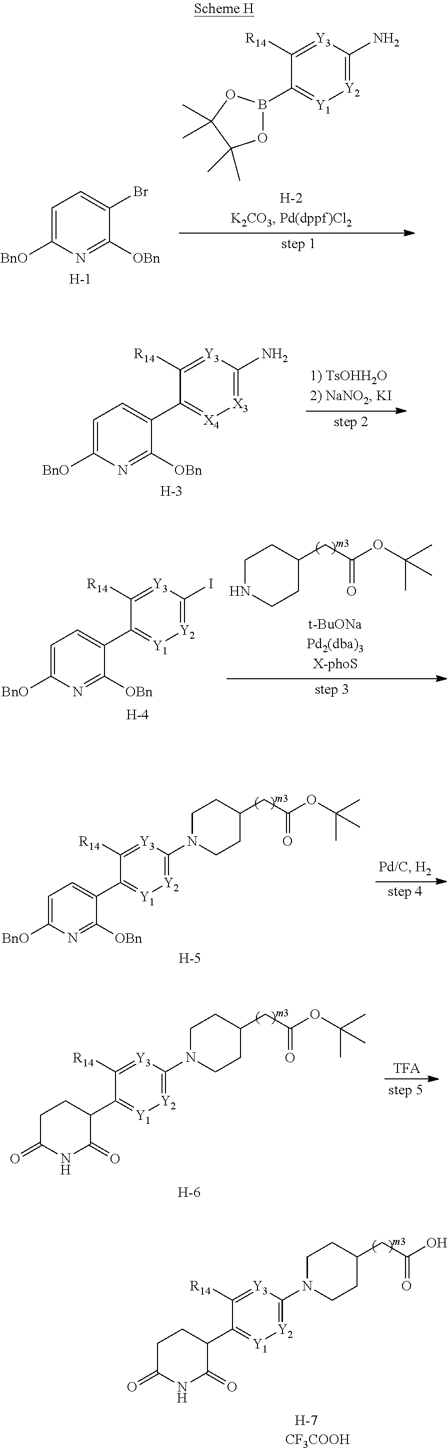

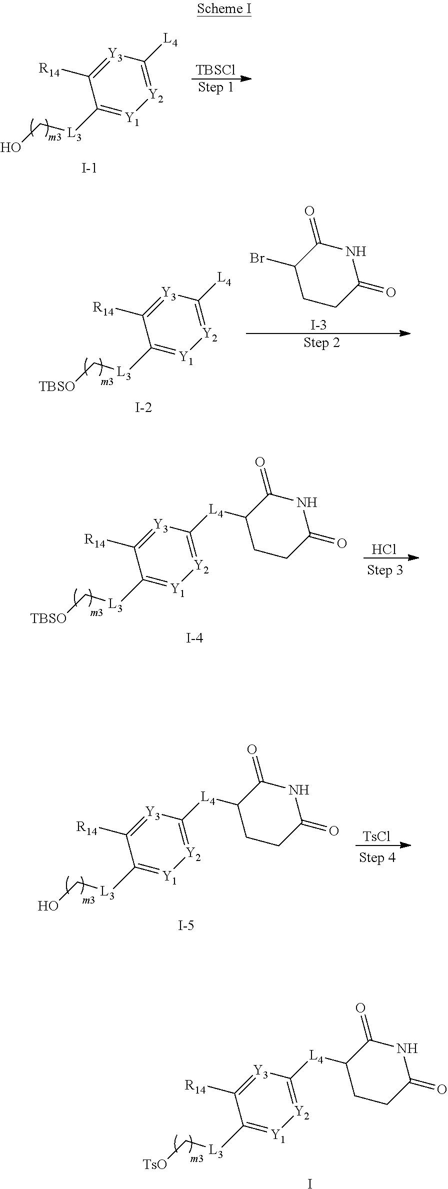

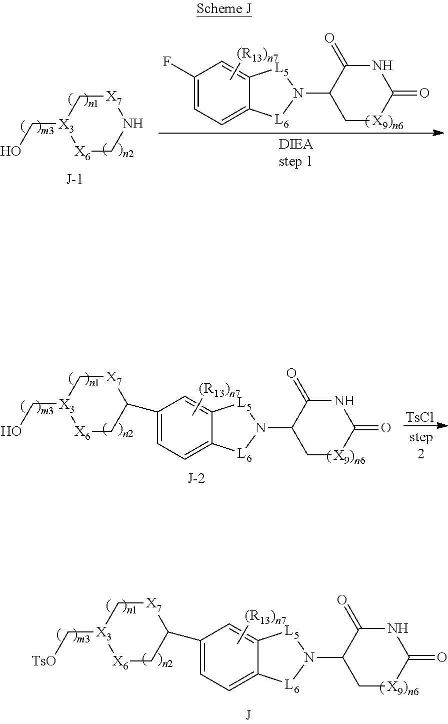

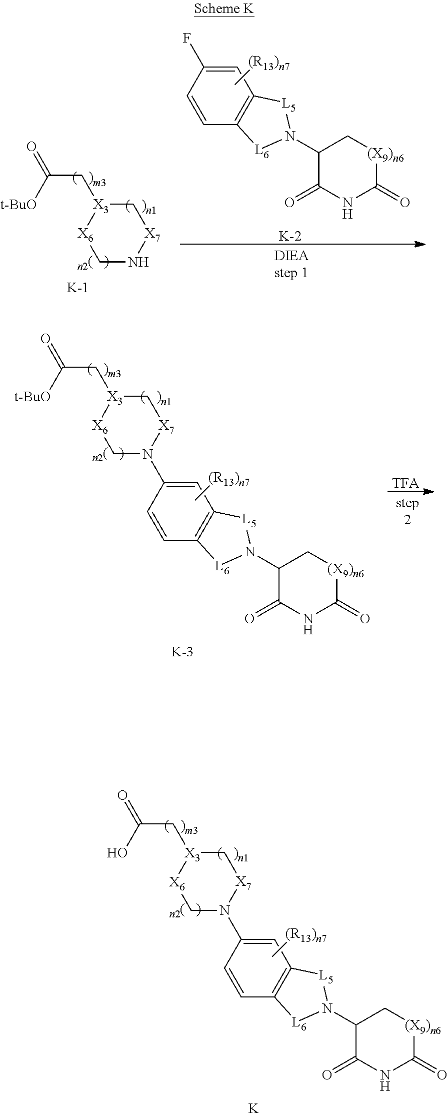

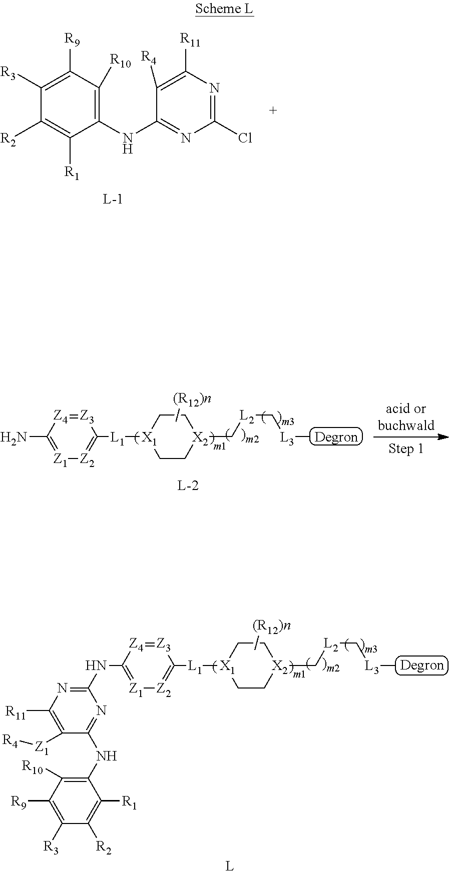

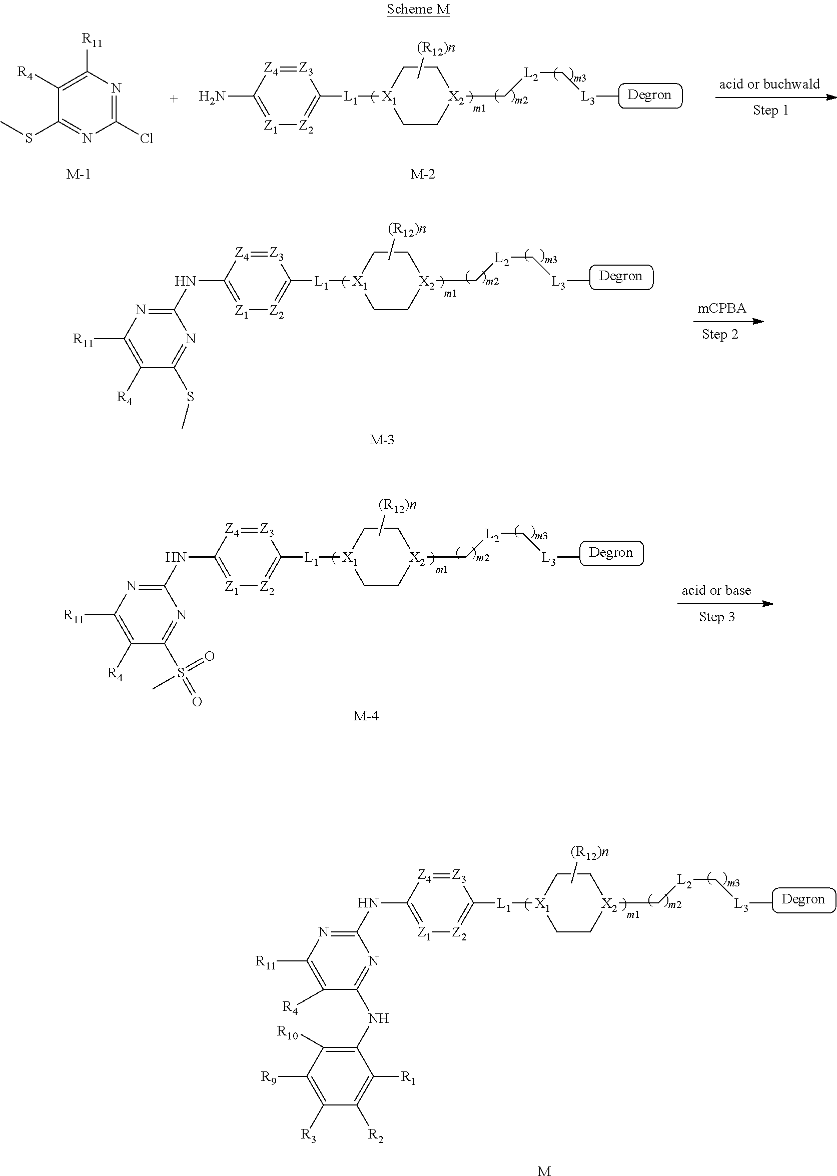

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on an Agilent instrument operating at 400 MHz. ¹HNMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D_{2O: 4.79} ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 n), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 n), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18) at a flow rate of 20 ml/min, injection volume 2 m1, at room temperature and UV Detection at 214 nm and 254 nm.

In the following examples, the abbreviations below are used:

| | |
|---|---|
| (BPin)₂ | 4,4,4′,4′,5,5,5′,5′-Octamethyl-2,2′-bi-1,3,2-dioxaborolane |
| Ac₂O | acetic anhydride |
| AcCl | Acetyl chloride |
| ACN or MeCN | Acetonitrile |
| AcOH or HOAc | Acetic acid |
| AcONa or NaOAc | Sodium acetate |
| Aq | Aqueous |
| BINAP | (±)-2,2′-Bis(diphenylphosphino)-1,1′-BINAPhthyl |
| Bn | benzyl |
| BnBr | Benzyl Bromide |
| Boc | t-Butyloxy carbonyl |
| BTEAC | Benzyltriethylammonium chloride |
| C:40691-33-6 | dichlorobis(tri-o-tolylphosphine)palladium(II) |
| Cbz | Benzyloxycarbonyl |
| DCM | Dichloromethane |
| Con. | Concentrated |
| DavePhos | 2′-(Dicyclohexylphosphino)-N,N-dimethyl-2-biphenylamine |
| DBU | 1,8-diazabicyclo[5.4.0]undec-7-ene |
| DCE | dichloroethane |
| DHP | 3,4-Dihydro-2H-pyran |
| DIBAL-H | Diisobutylaluminium hydride |
| DIEA or DIPEA | N,N-diisopropylethylamine |
| DMAP | 4-N,N-dimethylaminopyridine |
| DMP | Dess-Martin periodinane |
| DMF | N,N-Dimethylformamide |
| DMSO | Dimethyl sulfoxide |
| Dppf | 1,1″-bis(diphenylphosphino)ferrocene |
| EA or EtOAc | Ethyl acetate |
| EtOH | ethanol |
| FA | Formic acid |
| h or hr | Hour |
| HATU | 2-(7-Azabenzotriazol-1-yl)-N,N,N′,N′-tetramethyluronium |
| | hexafluorophosphate |
| HBTU | O-(7-Benzotriazole-1-yl)-N,N,N′,N′-tetramethyluronium |
| | hexafluorophosphate |
| Hex | Hexane |
| HPLC | High Performance Liquid Chromatography |
| hrs | hours |
| IBX | 2-Iodoxybenzoic acid |
| IPA | 2-propanol |
| i-PrOH | Isopropyl alcohol |
| KHMDS | Potassium bis(trimethylsilyl)amide |
| LiHMDS | Lithium bis(trimethylsilyl)amide |
| KOAc or AcOK | Potassium Acetate |
| MeCN or ACN | Acetonitrile |
| MeOH | Methanol |
| Min | Minutes |
| ms or MS | Mass spectrum |
| MsCl | Methanesulfonyl chloride |
| MsOH | Methanesulfonic acid |
| MTBE | Methyl tert-butyl ether |
| o/n | overnight |
| Pd(dppf)Cl₂ | [1,1′-Bis(diphenylphosphino)ferrocene]dichloropalladium(II) |
| Pd₂(dba)₃ | Tris(dibenzylideneacetone)dipalladium |
| PE | Petroleum ether |
| PhMe | Toluene |
| PMB | 4-Methoxybenzyl |
| PPA | Polyphosphoric acid |
| R.T. or r.t. | Room temperature |
| Rt | Retention time |
| Selectfluor | 1-Chloromethyl-4-fluoro-1,4-diazoniabicyclo[2.2.2]octane |
| | bis(tetrafluoroborate) |
| SEMCl | 2-(Trimethylsilyl)ethoxymethyl chloride |
| STAB | Sodium Triacetoxyborohydride; Sodium triacetoborohydride |
| Sat. | saturated |
| SFC | Supercritical Fluid Chromatography |
| TBAF | Tetra-butyl ammonium fluoride |
| TBDPS | tert-Butyldiphenylsilyl |
| TBS | tert-Butyldimethylsilyl |
| TBSCl | tert-Butyldimethylsilyl chloride |
| Ti(OiPr)₄ | Titanium tetraisopropanolate |
| t-Bu | tert-butyl |
| t-BuOH | tert-Butanol |
| t-BuONa | Sodium tert-butoxide |
| t-BuOK | Potassium tert-butoxide |
| TEA | Triethylamine |
| Tf₂O | Triflic anhydride |
| TFA | Trifluoroacetic acid |
| THF | Tetrahydrofuran |
| TLC | Thin layer chromatography |
| TMSOK | Potassium trimethylsilanolate |
| Ts | para-Toluenesulfonyl |
| TsCl | 4-Toluenesulfonyl chloride |
| TsOH | p-toluenesulfonic acid |
| TsOH,Py | Pyridinium toluene-4-sulphonate |
| Xphos or X-phos | 2-Dicyclohexylphosphino-2′,4′,6′-triisopropylbiphenyl |
| XantPhos | 4,5-Bis(diphenylphosphino)-9,9-dimethylxanthene |
| TCFH | N,N,N′,N′-Tetramethylchloroformamidinium hexafluorophosphate |
| CDI | 1,1′-Carbonyldiimidazole |

### Example 23: 3-(4-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)piperidin-1-yl)phenyl)piperidine-2,6-dione

### Step 1: tert-butyl 4-(1-(2-bromo-5-methoxy-4-nitrophenyl)piperidin-4-yl)piperazine-1-carboxylate

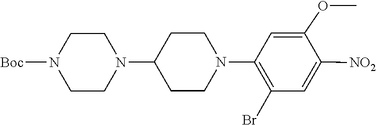 [SMILES: COc1cc(N2CCC(N3CCN(C(=O)OC(C)(C)C)CC3)CC2)c(Br)cc1[N+](=O)[O-]]

A mixture of 1-bromo-2-fluoro-4-methoxy-5-nitrobenzene (4 g, 16 mmol), tert-butyl 4-(piperidin-4-yl)piperazine-1-carboxylate (6.4 g, 24 mmol), K₂CO₃ (4.4 g, 32 mmol) in DMF (50 mL) was stirred in a flask at 80° C. overnight. The reaction mixture was allowed to cool down to room temperature. The resulting mixture was diluted with water and extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (1:1) to afford the product (7 g, 90%). [M+H]⁺=499.0.

Step 2: tert-butyl 4-(1-(5-methoxy-4-nitro-2-vinylphenyl)piperidin-4-yl)piperazine-1-carboxylate

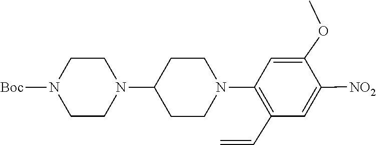 [SMILES: C=Cc1cc([N+](=O)[O-])c(OC)cc1N1CCC(N2CCN(C(=O)OC(C)(C)C)CC2)CC1]

A mixture of tert-butyl 4-(1-(2-bromo-5-methoxy-4-nitrophenyl)piperidin-4-yl)piperazine-1-carboxylate (7 g, 14 mmol), 4,4,5,5-tetramethyl-2-vinyl-1,3,2-dioxaborolane (4.3 g, 28 mmol), Pd(dppf)Cl₂ (1.1 g, 1.4 mmol) and K₃PO₄ (8.9 g, 42 mmol) in DMF (160 mL) and water (20 mL) was stirred in a flask at 90° C. under nitrogen atmosphere for 16 hrs. The reaction mixture was allowed to cool down to room temperature. The resulting mixture was extracted with EtOAc (3×1000 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (1:1) to afford the product (5 g, 80%). [M+H]⁺=447.0.

### Step 3: tert-butyl 4-(1-(4-amino-2-ethyl-5-methoxyphenyl)piperidin-4-yl)piperazine-1-carboxylate

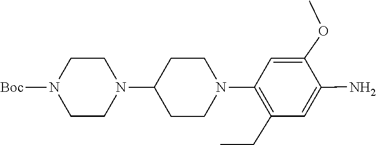 [SMILES: CCc1cc(N)c(OC)cc1N1CCC(N2CCN(C(=O)OC(C)(C)C)CC2)CC1]

To a stirred solution of tert-butyl 4-(1-(5-methoxy-4-nitro-2-vinylphenyl)piperidin-4-yl)piperazine-1-carboxylate (5 g, 11.2 mmol) in MeOH (100 mL) and DCM (20.00 mL) was added Pd/C (wet, 10%) (1 g) under nitrogen atmosphere. The resulting mixture was stirred for 16 hrs at room temperature under hydrogen atmosphere. The resulting mixture was filtered, the filter cake was washed with DCM/CH₃OH (10: 1, 200 mL). The filtrate was concentrated under reduced pressure to afford the product (4.0 g, 85.3%). [M+H]⁺=419.1.

### Step 4: (6-aminoquinoxalin-5-yl)dimethylphosphine oxide

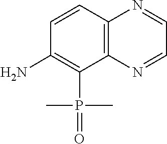

A mixture of 5-bromoquinoxalin-6-amine (10 g, 44.8 mmol), dimethylphosphine oxide (10.5 g, 134.5 mmol), Pd(OAc)₂ (1.0 g, 4.5 mmol) Xanphos (5.2 g, 9 mmol) and K₃PO₄ (28 g, 134 mmol) in DMF (250 mL) and water (50 mL) was stirred in a flask at 130° C. under nitrogen atmosphere for 16 hrs. The reaction mixture was allowed to cool down to room temperature. The resulting mixture was extracted with DCM (3×1000 mL). The combined organic layers were washed with brine (1000 mL), dried over anhydrous Na₂SO₄. After filtration, the filtrate was concentrated under reduced pressure. The residue was purified by silica gel column chromatography, eluted with DCM/MeOH (10:1) to afford the product (6 g, 60%), [M+H]⁺=222.0.

### Step 5: (6-((5-bromo-2-chloropyrimidin-4-yl)amino)quinoxalin-5-yl)dimethyl phosphine oxide

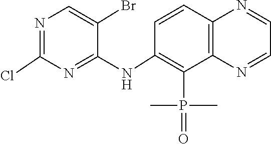 [SMILES: CP(C)(=O)c1c(Nc2nc(Cl)ncc2Br)ccc2nccnc12]

A mixture of (6-aminoquinoxalin-5-yl)dimethylphosphine oxide (6 g, 27.3 mmol), 5-bromo-2,4-dichloropyrimidine (12.3 g, 54.6 mmol) in THF (200 mL) was stirred in a flask at 0° C. under nitrogen atmosphere, 54 mL KHMDS (1M in THF) was added. The reaction mixture was allowed to warm up to room temperature for 2 hours. The reaction was quenched with water and the mixture was extracted with DCM, washed three times with saturated brine, dried over anhydrous Na₂SO₄. After filtration, the filtrate was concentrated under reduced pressure. The residue was purified by silica gel column chromatography, eluted with DCM/MeOH (10:1) to afford the product (4 g, 35%). [M+H]⁺=412.0.

### Step 6: (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide

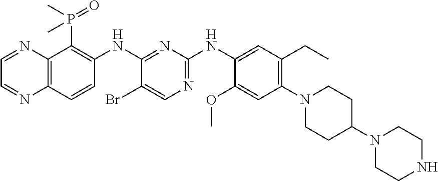 [SMILES: CCc1cc(Nc2ncc(Br)c(Nc3ccc4nccnc4c3P(C)(C)=O)n2)c(OC)cc1N1CCC(N2CCNCC2)CC1]

A mixture of (6-((5-bromo-2-chloropyrimidin-4-yl)amino)quinoxalin-5-yl)dimethyl phosphine oxide (2 g, 4.8 mmol), tert-butyl 4-(1-(4-amino-2-ethyl-5-methoxyphenyl)piperidin-4-yl)piperazine-1-carboxylate (2.6 g, 6.3 mmol) and MsOH (184 mg, 1.92 mmol) in t-BuOH (20 mL) was stirred in a flask at 90° C. under nitrogen atmosphere for overnight. The reaction mixture was concentrated under reduced pressure. The residue was purified by silica gel column chromatography to afford the product (2 g, 60%). [M+H]⁺=694.0.

### Step 7: 2,6-bis(benzyloxy)-3-(4-bromophenyl)pyridine

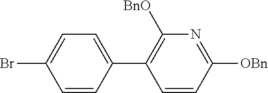 [SMILES: Brc1ccc(-c2ccc(OCc3ccccc3)nc2OCc2ccccc2)cc1]

To a stirred mixture of 2,6-bis(benzyloxy)-3-(4,4,5,5-tetramethyl-1,3,2-dioxaborolan-2-yl)pyridine (The intermediate can be prepared according to the way described in WO2017197046) (25 g, 59.9 mmol) and 4-bromoiodobenzene (20.3 g, 71.9 mmol) in dioxane (250 mL) and H₂O (50 mL) were added K₂CO₃ (16.6 g, 120 mmol) and Pd(dppf)Cl₂ (4.4 g, 6.0 mmol) at room temperature under nitrogen atmosphere. The resulting mixture was stirred for 16 h at 80° C. under nitrogen atmosphere. The reaction mixture was allowed to cool down to room temperature. The resulting mixture was extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (10:1) to afford the product (23 g, 86%); [M+H]⁺=446.2.

### Step 8: ethyl 2-(1-[4-[2,6-bis(benzyloxy)pyridin-3-yl]phenyl]piperidin-4-yl)acetate

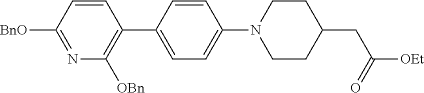 [SMILES: CCOC(=O)CC1CCN(c2ccc(-c3ccc(OCc4ccccc4)nc3OCc3ccccc3)cc2)CC1]

To a stirred solution of 2,6-bis(benzyloxy)-3-(4-bromophenyl)pyridine (15 g, 33.6 mmol) and ethyl 2-(piperidin-4-yl)acetate (8.6 g, 50.4 mmol) in 2-methyl-THF (150 mL) and H₂O (15 mL) were added Cs₂CO₃ (32.9 g, 100.8 mmol), DavePhos (2.7 g, 6.7 mmol) and Pd₂(dba)₃ (3.1 g, 3.4 mmol) at room temperature under nitrogen atmosphere. The resulting mixture was stirred for 16 h at 100° C. under nitrogen atmosphere. The mixture was allowed to cool down to room temperature. The resulting mixture was concentrated under reduced pressure. The residue was diluted with EtOAc (500 mL), washed with water (3×200 mL) and brine (200 mL). The organic layer was dried over anhydrous Na₂SO₄. After filtration, the filtrate was concentrated under reduced pressure. The residue was purified by silica gel column chromatography, eluted with PE/EtOAc (1:1) to afford the product (14 g, 78%); [M+H]⁺=537.3.

### Step 9: 2-(1-(4-(2,6-bis(benzyloxy)pyridin-3-yl)phenyl)piperidin-4-yl)ethan-1-ol

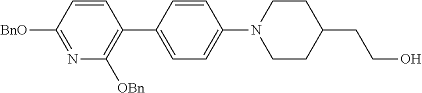 [SMILES: OCCC1CCN(c2ccc(-c3ccc(OCc4ccccc4)nc3OCc3ccccc3)cc2)CC1]

To a stirred solution of ethyl 2-(1-[4-[2,6-bis(benzyloxy)pyridin-3-yl]phenyl]piperidin-4-yl)acetate (13 g, 24.2 mmol) in THF (130 mL) was added LiAlH₄ (1 g, 26.6 mmol) in portions at 0° C. The resulting mixture was stirred for 2 h at room temperature. The reaction was quenched by the addition of water/ice (50 mL) at 0° C. The resulting mixture was extracted with EtOAc (3×50 mL). The combined organic layers were washed with brine (50 mL), dried over anhydrous Na₂SO₄. After filtration, the filtrate was concentrated under reduced pressure. The residue was purified by silica gel column chromatography, eluted with PE/EtOAc (1:2) to afford the product (11 g, 92%); [M+H]⁺=495.3.

### Step 10: 3-[4-[4-(2-hydroxyethyl)piperidin-1-yl]phenyl]piperidine-2,6-dione

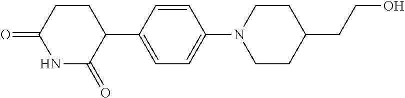 [SMILES: O=C1CCC(c2ccc(N3CCC(CCO)CC3)cc2)C(=O)N1]

To a stirred solution of 2-(1-(4-(2,6-bis(benzyloxy)pyridin-3-yl)phenyl)piperidin-4-yl)ethan-1-ol (10.5 g, 21.2 mmol) in EtOH (100 mL), EtOAc (100 mL) and DCM (20 mL) was added Pd/C (wet, 10%) (5 g) under nitrogen atmosphere. The resulting mixture was stirred for 16 h at room temperature under hydrogen atmosphere. The resulting mixture was filtered, the filter cake was washed with DCM/CH₃OH (10:1, 200 mL). The filtrate was concentrated under reduced pressure. The residue was purified by silica gel column chromatography, eluted with MeOH/DCM (1:10) to afford the product (5.1 g, 76%). [M+H]⁺=317.1.

### Step 11: 2-(1-(4-(2,6-dioxopiperidin-3-yl)phenyl)piperidin-4-yl)acetaldehyde

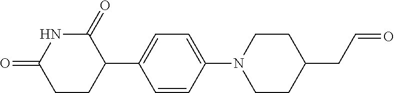 [SMILES: O=CCC1CCN(c2ccc(C3CCC(=O)NC3=O)cc2)CC1]

A mixture of 3-[4-[4-(2-hydroxyethyl)piperidin-1-yl]phenyl]piperidine-2,6-dione (100 mg, 0.32 mmol) and IBX (132 mg, 0.47 mmol) in DMSO (10 mL) was stirred in a flask at room temperature overnight. The reaction was quenched with water and the mixture was extracted with EtOAc, washed three times with saturated aqueous NaCl and twice with saturated aqueous NaHCO₃. The organic layer was dried over anhydrous Na₂SO₄ and evaporated in vacuum to afford the product (70 mg, 70%). [M+H]⁺=315.2.

### Step 12: 3-(4-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)piperidin-1-yl)phenyl)piperidine-2,6-dione

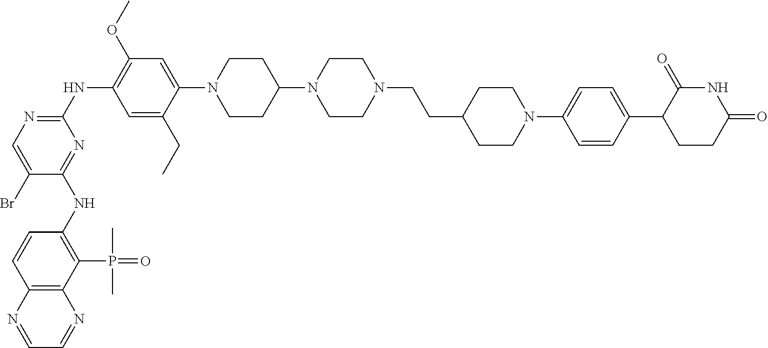 [SMILES: CCc1cc(Nc2ncc(Br)c(Nc3ccc4nccnc4c3P(C)(C)=O)n2)c(OC)cc1N1CCC(N2CCN(CCC3CCN(c4ccc(C5CCC(=O)NC5=O)cc4)CC3)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (40 mg, 0.057 mmol) and 2-(1-(4-(2,6-dioxopiperidin-3-yl)phenyl)piperidin-4-yl)acetaldehyde (21 mg, 0.069 mmol) and NaOAc (14 mg, 0.17 mmol) in chloromethane (4 mL) and EtOH (0.5 mL) was stirred in a flask at room temperature for 2 hour. The mixture was added NaBH₃CN (10 mg, 0.17 mmol) and stirred in a flask at room temperature 2 h.

Then the mixture was evaporated in vacuum to afford the crude product, which was purified with HPLC chromatography (0.1% FA in water: acetonitrile=90:10˜ 50:50 gradient elution) to give the product (15 mg, 27%). ¹H NMR (400 MHz, DMSO) δ 12.65 (s, 1H), 10.79 (s, 1H), 8.87 (d, J=9.1 Hz, 3H), 8.29 (d, J=12.4 Hz, 2H), 7.91 (s, 1H), 7.38 (s, 1H), 7.03 (d, J=7.8 Hz, 2H), 6.89 (d, J=8.4 Hz, 2H), 6.81 (s, 1H), 3.77 (s, 3H), 3.76-3.62 (m, 3H), 3.02 (s, 6H), 2.80-2.53 (m, 12H), 2.44-2.40 (m, 1H), 2.11 (s, 2H), 2.02 (d, J=14.3 Hz, 7H), 1.88 (s, 2H), 1.75 (d, J=11.0 Hz, 2H), 1.48-1.56 (m, 5H), 1.28-1.30 (m, 3H), 0.93-0.95 (m, 3H); [M+H]⁺=992.5.

### Example 1: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)phenyl)piperidine-2,6-dione

### Step 1: 2-(4-(4,4,5,5-tetramethyl-1,3,2-dioxaborolan-2-yl)phenyl)ethan-1-ol

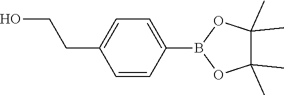 [SMILES: CC1(C)OB(c2ccc(CCO)cc2)OC1(C)C]

2-(4-bromophenyl)ethan-1-ol (20 g, 100 mmol), 4,4,4′,4′,5,5,5′,5′-Octamethyl-2,2′-bi-1,3,2-dioxaborolane (38.1 g, 150 mmol), Pd(dppf)Cl₂ (7.3 g, 10 mmol), KOAc (19.6 g, 200 mmol) were placed in dioxane (400 mL). The resulting mixture was then heated to reflux for 2 h. The mixture was cooled to room temperature, filtered off the solid and concentrated to afford crude product (28 g, crude), which was used directly without further purification. [M+H]⁺=249.2.

### Step 2: 2-(4-(2,6-bis(benzyloxy)pyridin-3-yl)phenyl)ethan-1-ol

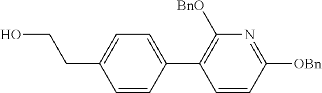 [SMILES: OCCc1ccc(-c2ccc(OCc3ccccc3)nc2OCc2ccccc2)cc1]

2-(4-(4,4,5,5-tetramethyl-1,3,2-dioxaborolan-2-yl)phenyl)ethan-1-ol (28 g, crude), Pd(dppf)Cl₂ (7.3 g, 10 mmol), 2,6-bis(benzyloxy)-3-bromopyridine (36.9 g, 100 mmol), Cs₂CO₃ (65.2 g, 200 mmol) were placed in Dioxane/water (300 mL, 10:1). The mixture was stirred at 100° C. overnight. Cooling the reaction to room temperature, filtered off the solid, the filtrate was concentrated and purified with SiO₂-gel column (eluted with EtOAc/Hexane=1:2) to give the crude product which was used directly in the next step. [M+H]⁺=412.2.

### Step 3: 3-(4-(2-hydroxyethyl)phenyl)piperidine-2,6-dione

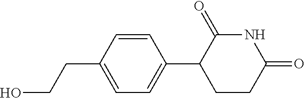 [SMILES: O=C1CCC(c2ccc(CCO)cc2)C(=O)N1]

2-(4-(2,6-bis(benzyloxy)pyridin-3-yl)phenyl)ethan-1-ol (the crude from last step) was dissolved in MeOH (500 mL), Pd/C (10%, w/w, 5 g) was added to the solution in one portion. The resulting mixture was stirred under H₂ atmosphere (1 atm) for overnight. Filtered off the solid, the filtrate concentrated to give the crude product. The crude was triturated with MTBE (50 mL) to give desire product (13.5 g, 57.9% over 3 steps). [M+H]⁺=234.1.

### Step 4: 2-(4-(2,6-dioxopiperidin-3-yl)phenyl)acetaldehyde

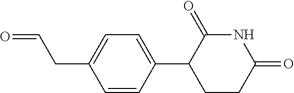 [SMILES: O=CCc1ccc(C2CCC(=O)NC2=O)cc1]

2-(4-(2,6-dioxopiperidin-3-yl)phenyl)acetaldehyde was prepared in a manner similar to that in Example 23 Step 11 from 3-(4-(2-hydroxyethyl)phenyl)piperidine-2,6-dione and IBX. [M+H]⁺=232.19.

### Step 5: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)phenyl)piperidine-2,6-dione

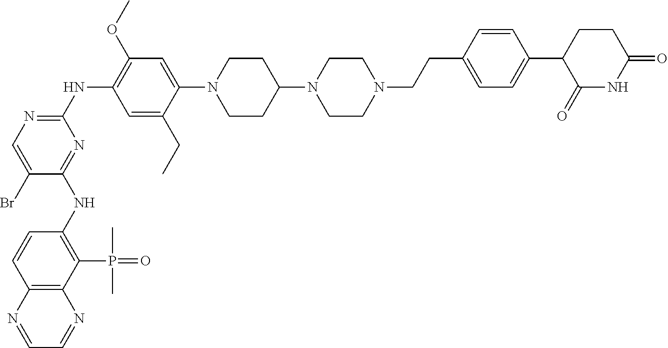 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)cc3)CC2)CC1]

The titled compound was synthesized in a manner similar to that in Example 23 step 12 from 2-(4-(2,6-dioxopiperidin-3-yl)phenyl)acetaldehyde and (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide. ¹H NMR (400 MHz, DMSO) δ 12.65 (s, 1H), 10.83 (s, 1H), 8.87 (d, J=4.2 Hz, 3H), 8.28 (d, J=8.8 Hz, 2H), 7.92 (s, 1H), 7.16 (d, J=19.2 Hz, 4H), 6.81 (s, 1H), 3.77 (s, 4H), 3.00-3.02 (m, 4H), 2.71-2.75 (m, 7H), 2.26-2.40 (m, 12H), 2.02 (m, 7H), 1.85-1.87 (m, 2H), 1.58 (d, J=11.1 Hz, 2H), 0.93 (s, 3H); [M+H]⁺=909.

### Example 2: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2-methoxyphenyl)piperidine-2,6-dione

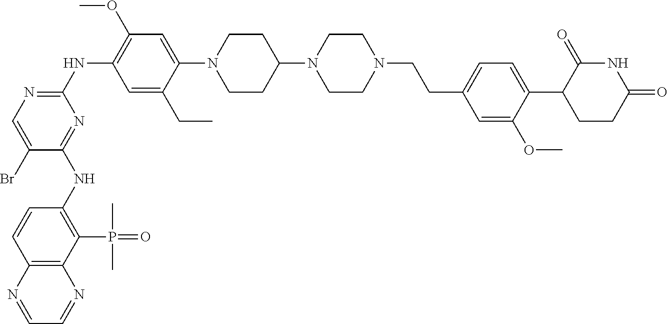 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(OC)c3)CC2)CC1]

The titled compound was synthesized in the procedures similar to Example 23. ¹H NMR (400 MHz, DMSO) δ 12.62 (s, 1H), 10.72 (s, 1H), 8.85 (s, 3H), 8.26 (d, J=12.7 Hz, 2H), 7.89 (s, 1H), 7.35 (s, 1H), 7.00 (s, 1H), 6.88 (s, 1H), 6.77 (d, J=14.8 Hz, 2H), 3.84 (s, 1H), 3.73 (d, J=13.8 Hz, 6H), 2.98 (s, 2H), 2.51-2.70 (m, 15H), 2.28-2.31 (m, 2H), 2.15-2.18 (s, 2H), 2.00 (d, J=14.3 Hz, 6H), 1.80-1.86 (m, 3H), 1.55-1.58 (m, 2H), 1.21 (s, 1H), 0.90 (s, 3H); [M+H]⁺=939.

### Example 3: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2-methylphenyl)piperidine-2,6-dione

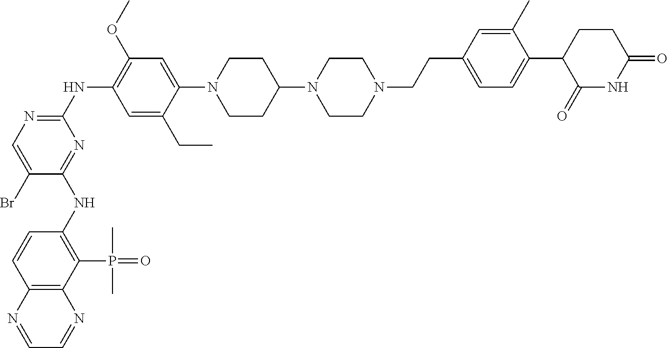 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(C)c3)CC2)CC1]

The titled compound was synthesized in the procedures similar to Example 23. ¹H NMR (400 MHz, DMSO) δ 12.66 (s, 1H), 10.84 (s, 1H), 8.87 (s, 3H), 8.30 (s, 2H), 7.92 (s, 1H), 7.37 (s, 1H), 7.01 (s, 3H), 6.81 (s, 1H), 4.00 (s, 1H), 3.78 (s, 3H), 3.02 (s, 3H), 2.70 (s, 7H), 2.05 (t, J=72.6 Hz, 20H), 1.59 (s, 3H), 1.25 (s, 2H), 0.93 (s, 4H); [M+H]⁺=923.

### Example 4: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2-chlorophenyl)piperidine-2,6-dione

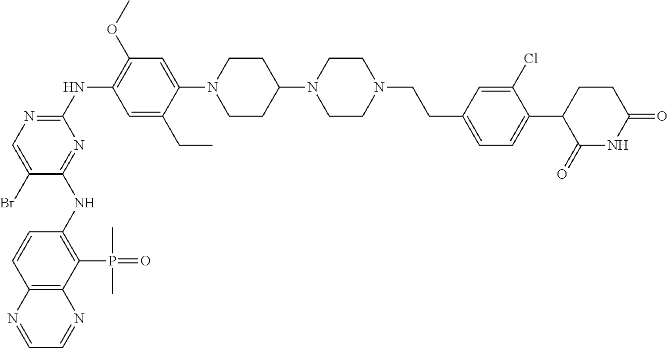 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(Cl)c3)CC2)CC1]

The titled compound was synthesized in the procedures similar to Example 23. ¹H NMR (400 MHz, DMSO) δ 12.62 (s, 1H), 10.88 (s, 1H), 8.85 (s, 3H), 8.26 (d, J=11.9 Hz, 2H), 7.88 (s, 1H), 7.34 (s, 2H), 7.19 (d, J=10.9 Hz, 2H), 6.79 (s, 1H), 4.12 (s, 1H), 3.75 (s, 3H), 2.98 (s, 3H), 2.50-2.80 (m, 13H), 2.30 (m, 4H), 1.90-2.01 (m, 10H), 1.57 (s, 3H), 0.90 (s, 3H); [M+H]⁺=943.

### Example 5: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2-fluorophenyl)piperidine-2,6-dione

### Step 1: 2-(4-bromo-3-fluorophenyl)ethan-1-ol

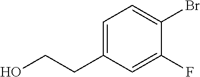 [SMILES: OCCc1ccc(Br)c(F)c1]

To a solution of 2-(4-bromo-3-fluorophenyl)acetic acid (45.0 g, 193 mmol) in THF (270 mL) was added BH₃-THF (1 M, 386 mL) at 0° C. Then the mixture was stirred at 20° C. for 2 hrs. Under cooling with ice, MeOH (250 mL) was dropwise added until there was no foaming in the system and the solvent was distilled off under reduced pressure. To the resulting reside, water (50.0 mL) was added for extraction with EtOAc (1000.0 mL). The combined organic phase was washed with brine (40.0 mL), dried over Na₂SO₄, filtered and concentrated in vacuum. 2-(4-bromo-3-fluorophenyl)ethan-1-ol (38.0 g, 89.8%) was obtained. ¹HNMR (400 MHz, CDCl₃-d) δ ppm 7.45 (t, J=7.72 Hz, 1H), 7.00 (dd, J=9.48, 1.76 Hz, 1H), 6.86-6.92 (m, 1H), 3.82 (t, J=6.50 Hz, 2H), 2.80 (t, J=6.50 Hz, 2H), 2.03 (s, 1H); [M+H]⁺=219.

### Step 2: (4-bromo-3-fluorophenethoxy)(tert-butyl)dimethylsilane

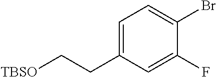 [SMILES: CC(C)(C)[Si](C)(C)OCCc1ccc(Br)c(F)c1]

To a solution of 2-(4-bromo-3-fluorophenyl)ethan-1-ol (38.0 g, 173 mmol) in DCM (210 mL) was added imidazole (17.7 g, 260 mmol) at 20° C. TBSCl (36.6 g, 242 mmol, 29.7 mL) was added to the reaction mixture at 0° C. Then the mixture was stirred at 20° C. for 3 hrs. Then the mixture was adjusted to pH=6 with 5% citric acid (180 mL), and extracted with DCM (150 mL). The organic phase was adjusted to pH=8 with NaHCO₃ and then aqueous phase was extracted with DCM (100 mL). The combined organic phase was washed with brine (150 mL), dried over Na₂SO₄, filtered and concentrated in vacuum. (4-bromo-3-fluorophenethoxy)(tert-butyl)dimethylsilane (52.0 g, 156 mmol) was obtained. ¹HNMR (400 MHz, CDCl₃-d) δ ppm 7.43 (t, J=7.72 Hz, 1H), 7.00 (dd, J=9.56, 1.87 Hz, 1H), 6.89 (dd, J=8.00, 1.87 Hz, 1H), 3.80 (t, J=6.48 Hz, 2H), 2.78 (t, J=6.48 Hz, 2H), 0.84-0.89 (m, 9H), −0.05-0.01 (m, 6H); [M+H]⁺=333.

### Step 3: 2,6-bis(benzyloxy)-3-(4-(2-((tert-butyldimethylsilyl)oxy)ethyl)-2-fluorophenyl)pyridine

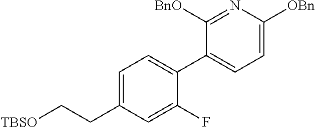 [SMILES: CC(C)(C)[Si](C)(C)OCCc1ccc(-c2ccc(OCc3ccccc3)nc2OCc2ccccc2)c(F)c1]

To a solution of (4-bromo-3-fluorophenethoxy)(tert-butyl)dimethylsilane (52.0 g, 156 mmol) and 2,6-bis(benzyloxy)-3-(4,4,5,5-tetramethyl-1,3,2-dioxaborolan-2-yl)pyridine (65.1 g, 156 mmol) in dioxane (320 mL) was added KOAc (45.9 g, 468 mmol) at 20° C. Pd(dppf)Cl₂ (11.4 g, 15.6 mmol) was added to the mixture at 20° C. The suspension was degassed under vacuum and purged with N₂ three times. Then the mixture was stirred at 90° C. for 16 hrs. Water (160 mL) was poured into the mixture, extrated with EtOAc (100 mL). The combined organic phase was washed with brine (100 mL), dried over Na₂SO₄, filtered and concentrated in vacuum. The residue was purified by column chromatography. 2,6-bis(benzyloxy)-3-(4-(2-((tert-butyldimethylsilyl)oxy)ethyl)-2-fluorophenyl)pyridine (32.0 g, 37.8%) was obtained. ¹HNMR (400 MHz, CDCl₃-d) δ ppm 7.55 (dd, J=8.04, 0.99 Hz, 1H), 7.43-7.47 (m, 2H), 7.33-7.42 (m, 7H), 7.25-7.33 (m, 3H), 6.98-7.05 (m, 2H), 5.40 (d, J=18.4 Hz, 4H), 3.87 (t, J=6.84 Hz, 1H), 3.84-3.89 (m, 1 H), 2.86 (t, J=6.84 Hz, 2H), 0.88-0.92 (m, 9H), 0.01-0.03 (m, 6H); [M+H]⁺=544.

### Step 4: 3-(4-(2-((tert-butyldimethylsilyl)oxy)ethyl)-2-fluorophenyl)piperidine-2,6-dione

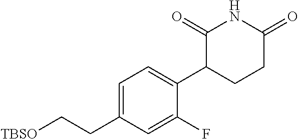 [SMILES: CC(C)(C)[Si](C)(C)OCCc1ccc(C2CCC(=O)NC2=O)c(F)c1]

To a solution of 2,6-bis(benzyloxy)-3-(4-(2-((tert-butyldimethylsilyl)oxy)ethyl)-2-fluorophenyl)pyridine (32.0 g, 58.8 mmol) in THF (50.0 mL) was added Pd/C (0.800 g, 10.0% purity) under Ar at 20° C. The suspension was degassed and purged with H₂ for 3 times. The mixture was stirred under H₂ (50 Psi) at 50° C. for 16 hrs. The suspension was filtered through a pad of celite and the filter cake was washed with THF (200 mL×3). The combined filtrates were concentrated to dryness to give crude product. The crude product was triturated with petroleum ether (50.0 mL) at 20° C. for 1 hrs. 3-(4-(2-((tert-butyldimethylsilyl)oxy)ethyl)-2-fluorophenyl)piperidine-2,6-dione (12.0 g, 55.7%) was obtained. ¹HNMR (400 MHz, CDCl₃-d) δ ppm 7.06-7.12 (m, 1H) 7.93 (br s, 1H), 6.96-7.04 (m, 2H), 3.91 (dd, J=11.2, 5.04 Hz, 1H), 3.81 (t, J=6.80 Hz, 2 H), 2.82 (t, J=6.80 Hz, 2H), 2.58-2.73 (m, 2H), 2.18-2.34 (m, 2H), 0.87 (s, 9H), 0.00 (s, 6H); [M+H]+=366.

### Step 5: 3-(2-fluoro-4-(2-hydroxyethyl)phenyl)piperidine-2,6-dione

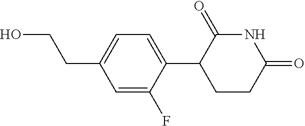 [SMILES: O=C1CCC(c2ccc(CCO)cc2F)C(=O)N1]

To a solution of 3-(4-(2-((tert-butyldimethylsilyl)oxy)ethyl)-2-fluorophenyl)piperidine-2,6-dione (12.0 g, 32.8 mmol) in MeOH (60 mL) was added HCl (12 M, 6 mL) at 20° C. Then the mixture was stirred at 20° C. for 3 hrs. Water (60 mL) was poured into the mixture, extracted with EtOAc (40 mL). The combined organic phase was washed with brine (40 mL), dried over Na₂SO₄, filtered and concentrated in vacuum. The combined crude product was purified by re-crystallization from toluene (32.0 mL) at 100° C. 3-(2-fluoro-4-(2-hydroxyethyl)phenyl)piperidine-2,6-dione (6.50 g, 78.8%) was obtained. ¹HNMR (400 MHz, DMSO-d₆) δ ppm 10.8 (s, 1H), 7.19 (t, J=7.84 Hz, 1H), 6.99-7.08 (m, 2H), 4.67 (t, J=5.18 Hz, 1H), 3.99 (dd, J=12.6, 4.74 Hz, 1H), 3.55-3.66 (m, 2H), 2.68-2.75 (m, 3H), 2.18 (qd, J=12.8, 3.86 Hz, 1H), 1.93-2.03 (m, 1H); [M+H]⁺=252.

### Step 6: 2-(4-(2,6-dioxopiperidin-3-yl)-3-fluorophenyl)acetaldehyde

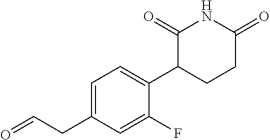 [SMILES: O=CCc1ccc(C2CCC(=O)NC2=O)c(F)c1]

To a solution of 3-(2-fluoro-4-(2-hydroxyethyl)phenyl)piperidine-2,6-dione (200 mg, 0.8 mmol) in DMSO (10 mL) was added IBX (338 mg, 1.2 mmol). The mixture was stirred in a flask at rt overnight. After being determined the reaction to be completed by LCMS, the mixture was extracted with EA (30 mL * 3). The combined organic phase was dried over anhydrous Na₂SO₄, and evaporated in vacuum to afford the crude product (100 mg, crude), which was used for next step without further purification. [M+H]⁺=250.

### Step 7: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2-fluorophenyl)piperidine-2,6-dione

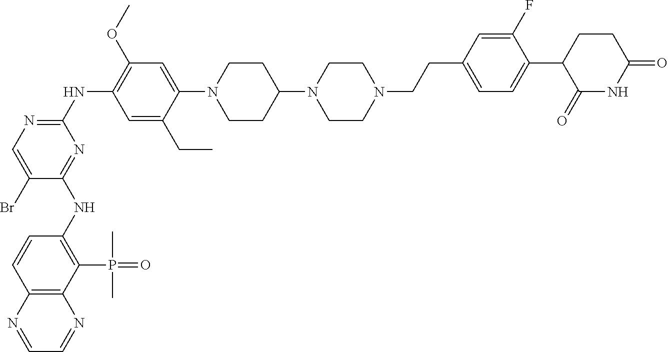 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(F)c3)CC2)CC1]

The titled compound was synthesized in a manner similar to that in Example 23 step 12 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 2-(4-(2,6-dioxopiperidin-3-yl)-3-fluorophenyl)acetaldehyde. ¹H NMR (400 MHz, DMSO) δ 12.63 (s, 1H), 10.85 (s, 1H), 8.86-8.74 (m, 2H), 8.25 (s, 1H), 7.87 (s, 1H), 7.35 (s, 1H), 7.18 (t, J=8.0 Hz, 1H), 7.04 (dd, J=17.1, 10.2 Hz, 2H), 6.79 (s, 1H), 4.15-3.91 (m, 1H), 3.75 (s, 2H), 2.98 (s, 2H), 2.80-2.61 (m, 4H), 2.51-2.60 (m, 4H), 2.41-2.50 (m, 7H), 2.1-2.35 (m, 5H), 2.00 (m, 6H), 1.80-1.84 (m, 2H), 1.55-1.59 (m, 3H), 1.21 (s, 3H), 0.91 (s, 3H); [M+H]⁺=927.

### Example 7: 3-(4-(5-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)pentyl)phenyl)piperidine-2,6-dione

### Step 1: 5-(4-bromophenyl)pent-4-yn-1-ol

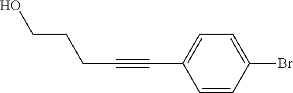 [SMILES: OCCCC#Cc1ccc(Br)cc1]

A mixture of 1-bromo-4-iodobenzene (3.0 g, 10.6 mmol), pent-4-yn-1-ol (0.98 g, 11.7 mmol), CuI (204 mg, 1.06 mmol), Pd(PPh₃)₂Cl₂ (373 mg, 0.53 mmol) and piperidine (1.8 g, 21.2 mmol) in toluene (30 mL) was stirred in a flask at 40° C. overnight under N₂. The mixture was evaporated in vacuum to afford the crude product, which was further purified with silica gel column chromatography (PE: EA=100: 0˜ 2: 1 gradient elution) to give the product (1.5 g, 59%). [M+H]⁺=239.0.

### Step 2: 5-(4-(4,4,5,5-tetramethyl-1,3,2-dioxaborolan-2-yl)phenyl)pent-4-yn-1-ol

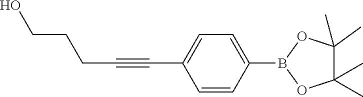 [SMILES: CC1(C)OB(c2ccc(C#CCCCO)cc2)OC1(C)C]

A mixture of 5-(4-bromophenyl)pent-4-yn-1-ol (1.2 g, 5.0 mmol), 4,4,4′,4′,5,5,5′,5′-octamethyl-2,2′-bi(1,3,2-dioxaborolane) (1.5 g, 6.0 mmol), Pd(dppf)Cl₂ (367 mg, 0.5 mmol) and KOAc (1.5 g, 15.0 mmol) in 1,4-dioxane (30 mL) was stirred in a flask at 80° C. for 1 h. The mixture was evaporated in vacuum to afford the crude product, which was further purified with silica gel column chromatography (PE: EA=100: 0˜ 4: 1 gradient elution) to give the title product (1.4 g, 97%). [M+H]⁺=287.2.

### Step 3: 5-(4-(2,6-bis(benzyloxy)pyridin-3-yl)phenyl)pent-4-yn-1-ol

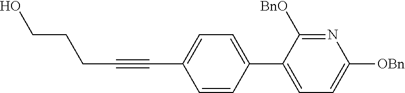 [SMILES: OCCCC#Cc1ccc(-c2ccc(OCc3ccccc3)nc2OCc2ccccc2)cc1]

A mixture of 5-(4-(4,4,5,5-tetramethyl-1,3,2-dioxaborolan-2-yl)phenyl)pent-4-yn-1-ol (1.4 g, 4.9 mmol), 2,6-bis(benzyloxy)-3-bromopyridine (1.8 g, 4.9 mmol), Pd(dppf)Cl₂ (366 mg, 0.5 mmol) and Cs₂CO₃ (2.5 g, 7.7 mmol) in 1,4-dioxane (50 mL) and H₂O (10 mL) was stirred in a flask at 80° C. for 2 h. The mixture was evaporated in vacuum to afford the crude product, which was further purified with silica gel column chromatography (PE: EA=100: 0˜ 2: 1 gradient elution) to give the product (2.0 g, 91%). [M+H]+=450.2.

### Step 4: 3-(4-(5-hydroxypentyl)phenyl)piperidine-2,6-dione

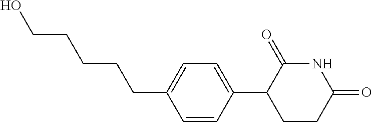 [SMILES: O=C1CCC(c2ccc(CCCCCO)cc2)C(=O)N1]

Under N₂, to a solution of 5-(4-(2,6-bis(benzyloxy)pyridin-3-yl)phenyl)pent-4-yn-1-ol (2.0 g, 4.4 mmol) in MeOH (100 mL) was added Pd/C (400 mg, 10%) at 25° C. And then the mixture was exchanged with H₂ two times and stirred under H₂ atmosphere at 25° C. for 48 h. Reaction was monitored by HPLC. The mixture was filtered through a pad of Celite and washed with MeOH (50 mL). The filtrate was concentrated under vacuum to obtain the product (1.2 g, 97%). [M+H]⁺=276.2.

### Step 5: 5-(4-(2,6-dioxopiperidin-3-yl)phenyl)pentanal

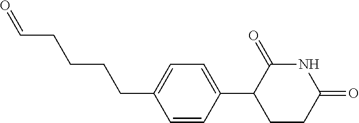 [SMILES: O=CCCCCc1ccc(C2CCC(=O)NC2=O)cc1]

To a solution of 3-(4-(5-hydroxypentyl)phenyl)piperidine-2,6-dione (100 mg, 0.36 mmol) in DMSO (3.0 mL) was added IBX (203 mg, 0.72 mmol) in portions at 25° C. The mixture was stirred at 25° C. for 4 h. Water (20.0 mL) was added and the resulting solution was extracted with EtOAc (2×30 mL). The combined organic layer was washed with brine (3×20 mL), dried over Na₂SO₄ and concentrated under vacuum to afford the title product (80 mg, 81%). [M+H]⁺=274.2.

### Step 6: 3-(4-(5-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)pentyl)phenyl)piperidine-2,6-dione

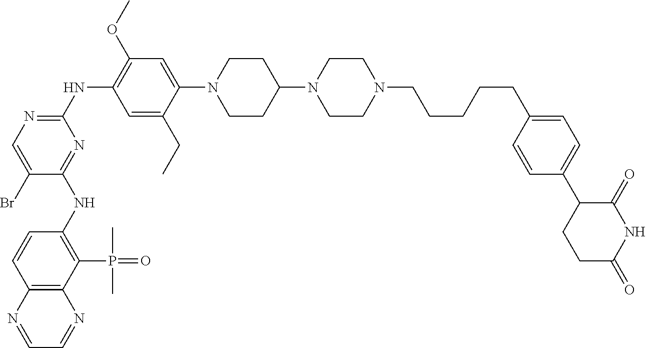 [SMILES: CCc1cc(Nc2ncc(Br)c(Nc3ccc4nccnc4c3P(C)(C)=O)n2)c(OC)cc1N1CCC(N2CCN(CCCCCc3ccc(C4CCC(=O)NC4=O)cc3)CC2)CC1]

The titled compound was prepared in a manner similar to that in Example 23 step 12. ¹H NMR (400 MHz, DMSO) δ 12.64 (s, 1H), 10.82 (s, 1H), 8.87 (s, 2H), 8.27 (s, 2H), 7.91 (s, 1H), 7.37 (s, 1H), 7.14 (d, J=7.9 Hz, 3H), 6.81 (s, 1H), 3.77 (s, 3H), 2.93-3.00 (m, 4H), 2.77-2.52 (m, 14H), 2.05-2.30 (m, 8H), 2.02 (d, J=14.1 Hz, 6H), 1.84 (s, 2H), 1.58 (s, 4H), 1.44 (s, 2H), 1.30 (s, 2H), 0.92 (s, 3H); [M+H]⁺=951.

### Example 13: 3-(6-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)pyridin-3-yl)piperidine-2,6-dione

### Step 1: 2-(5-(4,4,5,5-tetramethyl-1,3,2-dioxaborolan-2-yl)pyridin-2-yl)ethan-1-ol

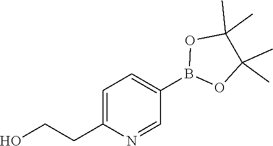 [SMILES: CC1(C)OB(c2ccc(CCO)nc2)OC1(C)C]

2-(5-bromopyridin-2-yl)ethan-1-ol (19 g, 94.5 mmol), 4,4,4′,4′,5,5,5′,5′-octamethyl-2,2′-bi(1,3,2-dioxaborolane) (26.4 g, 104 mmol), Pd(dppf)Cl₂ (6.9 g, 9.45 mmol), KOAc (18.5 g, 189 mmol) were placed in dioxane (300 mL). The resulting mixture was then heated to reflux for 16 h. The mixture was cooled to room temperature, filtered off the solid and concentrated to afford crude product (20 g, crude), which was used directly without further purification. [M+H]⁺=250.15.

### Step 2: 2-(2′,6′-bis(benzyloxy)-[3,3′-bipyridin]-6-yl)ethan-1-ol

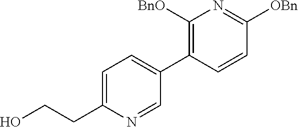 [SMILES: OCCc1ccc(-c2ccc(OCc3ccccc3)nc2OCc2ccccc2)cn1]

2-(5-(4,4,5,5-tetramethyl-1,3,2-dioxaborolan-2-yl)pyridin-2-yl)ethan-1-ol (20 g, crude), Pd(dppf)Cl₂ (4.5 g, 6.18 mmol), 2,6-bis(benzyloxy)-3-bromopyridine (22.9 g, 61.8 mmol), Cs₂CO₃ (40.2 g, 123.6 mmol) were placed in dioxane/water (300 mL, 10:1). The mixture was stirred at 100° C. for 16 h. Cooling the reaction to room temperature, filtered off the solid, the filtrate was concentrated and purified with SiO₂-gel column (eluted with EtOAc/Hexane=1:1) to give the product (19 g, 48.8% for over 2 steps). [M+H]+=413.18.

### Step 3: 3-(6-(2-hydroxyethyl)pyridin-3-yl)piperidine-2,6-dione

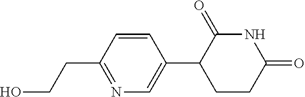 [SMILES: O=C1CCC(c2ccc(CCO)nc2)C(=O)N1]

2-(2′,6′-bis(benzyloxy)-[3,3′-bipyridin]-6-yl)ethan-1-ol (10 g, 24.3 mmol) was dissolved in MeOH (100 mL), Pd/C (10%, 1 g) was added to the solution in one portion. The resulting mixture was stirred under hydrogen atmosphere (1 atm) for 16 h. Filtered off the solid, concentrated to give the crude product. The crude was triturated with MTBE to give the desired product (1.7 g, 30%). [M+H]⁺=235.1.

### Step 4: 2-(5-(2,6-dioxopiperidin-3-yl)pyridin-2-yl)ethyl 4-methylbenzenesulfonate

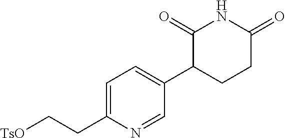 [SMILES: Cc1ccc(S(=O)(=O)OCCc2ccc(C3CCC(=O)NC3=O)cn2)cc1]

Into a flask, was placed 3-(6-(2-hydroxyethyl)pyridin-3-yl)piperidine-2,6-dione (269 mg, 1.15 mmol), DCM (8.00 mL), TEA (174.7 mg, 1.726 mmol), TsCl (328.6 mg, 1.72 mmol). The resulting solution was stirred overnight at room temperature. The resulting mixture was concentrated under vacuum. The residue was applied onto a silica gel column with dichloromethane and methanol (10:1) to afford the product (150 mg, 34%). [M+H]⁺=389.2.

### Step 5: 3-(6-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)pyridin-3-yl)piperidine-2,6-dione

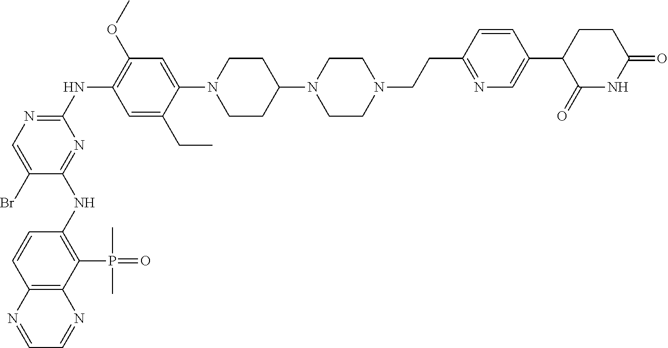 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)cn3)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (40 mg, 0.057 mmol), 2-(5-(2,6-dioxopiperidin-3-yl)pyridin-2-yl)ethyl 4-methylbenzenesulfonate (33 mg, 0.085 mmol), KI (14.2 mg, 0.085 mmol) and DIEA (14.7 mg, 0.114 mmol) in acetonitrile (4 mL) was stirred in a flask at 85° C. for 12 hours. The reaction was quenched with water and the mixture was extracted with DCM, washed with saturated brine. dried over anhydrous Na₂SO₄. After filtration, the filtrate was concentrated under reduced pressure, The residue was purified by HPLC chromatography to give the product (2.1 mg, 4%). ¹H NMR (400 MHz, DMSO) δ 12.63 (s, 1H), 10.88 (s, 1H), 8.85 (s, 3H), 8.32 (s, 1H), 8.26 (s, 2H), 7.94-7.84 (m, 1H), 7.53 (s, 1H), 7.35 (s, 1H), 7.25 (s, 1H), 6.79 (s, 1H), 3.86 (s, 1H), 3.75 (s, 3H), 2.98 (s, 2H), 2.85 (s, 2H), 2.67 (d, J=11.4 Hz, 5H), 2.50 (m, 3H), 2.10-2.40 (m, 4H), 2.00 (d, J=14.1 Hz, 7H), 1.80-1.85 (m, 3H), 1.55-1.58 (m, 3H), 1.21 (s, 3H), 0.90 (s, 3H); [M+H]⁺=910.

### Example 18: 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propoxy)phenyl)piperidine-2,6-dione

### Step 1: 4-(2,6-bis(benzyloxy)pyridin-3-yl)phenol

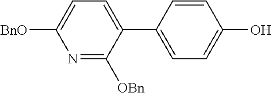 [SMILES: Oc1ccc(-c2ccc(OCc3ccccc3)nc2OCc2ccccc2)cc1]

A solution of 2,6-bis(benzyloxy)-3-bromopyridine (100 g, 0.27 mol) and (4-hydroxyphenyl)boronic acid (48.4 g, 0.35 mol) in dioxane (1.2 L) and water (120 mL) was added Pd(dppf)Cl₂ (20 g, 0.027 mol). The mixture was heated to 100° C. for 4 hours. The reaction mixture concentrated and diluted with EA (1.5 L). The organic layer was separated and washed with water (300 mL) and brine (300 mL), and then evaporated to dryness, which was purified by silica gel chromatography (PE:EA=20/1) to give product (72.2 g, 70.6%). [M−H]⁻=382.

### Step 2: 3-(4-hydroxyphenyl)piperidine-2,6-dione

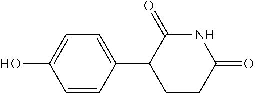 [SMILES: O=C1CCC(c2ccc(O)cc2)C(=O)N1]

To a solution of 4-(2,6-bis(benzyloxy)pyridin-3-yl)phenol (72.2 g, 0.189 mol) in methanol (1.5 L) was added Pd/C (50% w/t H₂O, 8 g). The mixture was charged with 1 atm H₂ and heated to 40° C. for overnight. The mixture was filtered and the filtrate was evaporated to give crude product (35.0 g, 90%). [M−H]⁻=204.

### Step 3: 4-(2,6-dioxopiperidin-3-yl)phenyl acetate

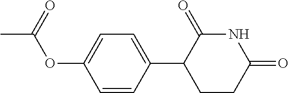 [SMILES: CC(=O)Oc1ccc(C2CCC(=O)NC2=O)cc1]

To a solution of 3-(4-hydroxyphenyl)piperidine-2,6-dione (35 g, 0.17 mol) in DCM (600 mL) was added Ac₂O (17.3 g, 0.17 mol) and Et₃N (20.2 g, 0.2 mol) at 0° C. The solution was warmed to room temperature overnight. The mixture was diluted with water. The organic layer was separated and washed with water (3*200 mL) and brine (200 mL) and then evaporated to give crude 4-(2,6-dioxopiperidin-3-yl)phenyl acetate which was re-crystallized from EtOAc and PE to give the product (38.3 g, 91.2%). [M−H]⁻=246.

### Step 4: 4-(2,6-dioxo-1-((2-(trimethylsilyl)ethoxy)methyl)piperidin-3-yl)phenyl acetate

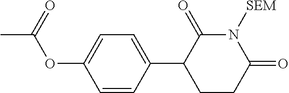 [SMILES: CC(=O)Oc1ccc(C2CCC(=O)N(COCC[Si](C)(C)C)C2=O)cc1]

To the solution of 4-(2,6-dioxopiperidin-3-yl)phenyl acetate (38.3 g, 0.155 mol) in THF (400 mL) was slowly added NaH (60% w/t in mineral oil, 6.2 g, 155 mol) at 0° C. Then SEM-C1 (25.9 g, 0.155 mol) was added at the same temperature. After completion, the mixture was stirred overnight at room temperature and then quenched with water (100 mL). Extracted with EA (2*300 mL), the organic layer was separated, washed with water (3*200 mL) and brine (200 mL), and then evaporated. The crude material was purified by silica gel chromatography (PE/EA=5/1) to give the product (35.2 g, 60%). [M+H]⁺=378.

### Step 5: 3-(4-hydroxyphenyl)-1-((2-(trimethylsilyl)ethoxy)methyl)piperidine-2,6-dione

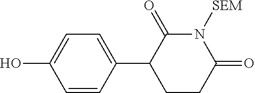 [SMILES: C[Si](C)(C)CCOCN1C(=O)CCC(c2ccc(O)cc2)C1=O]

To the solution of 4-(2,6-dioxo-1-((2-(trimethylsilyl)ethoxy)methyl)piperidin-3-yl)phenyl acetate (35.2 g, 0.093 mol) in methanol (500 mL) was added AcCl (7.8 g, 0.1 mol) at 0° C. The mixture was stirred overnight at room temperature and then poured into water (200 mL). The mixture was evaporated and extracted with DCM (2*300 mL). The organic solvent was washed with water and brine, and then evaporated. The crude material was purified by silica gel chromatography (PE/EA=3/1) to afford the product (20.6 g, 66%). ¹HNMR (400 MHz, CDCl₃): 7.04 (d, 2H, J=8.8 Hz), 6.79 (d, 2H, J=8.8 Hz), 5.28 (s, 2H), 3.81-3.77 (m, 1H), 3.67-3.62 (m, 2H), 2.81-2.71 (m, 2H), 2.23-2.18 (m, 2H), 0.98-0.94 (m, 2H), 0.00 (s, 9H), [M−H]⁻=334.

### Step 6: 3-(4-(3-iodopropoxy)phenyl)-1-((2-(trimethylsilyl)ethoxy)methyl)piperidine-2,6-dione

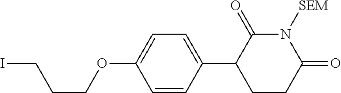 [SMILES: C[Si](C)(C)CCOCN1C(=O)CCC(c2ccc(OCCCI)cc2)C1=O]

3-(4-hydroxyphenyl)-1-((2-(trimethylsilyl)ethoxy)methyl)piperidine-2,6-dione (1.67 g, 5.0 mmol) was dissolved in acetone (4.5 ml). 1,3-Diiodopropane (1.15 ml, 10 mmol) and K₂CO₃ (1.38 g, 10 mmol) were added and the mixture was stirred at 50° C. for 16 h. The mixture was diluted with diethyl ether, washed with water and dried over MgSO₄. The crude material was purified by silica gel chromatography (PE/EA=3/1) to afford the product (0.8 g, 32%). [M+H]⁺=504.

### Step 7: 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propoxy)phenyl)-1-((2-(trimethylsilyl)ethoxy)methyl)piperidine-2,6-dione

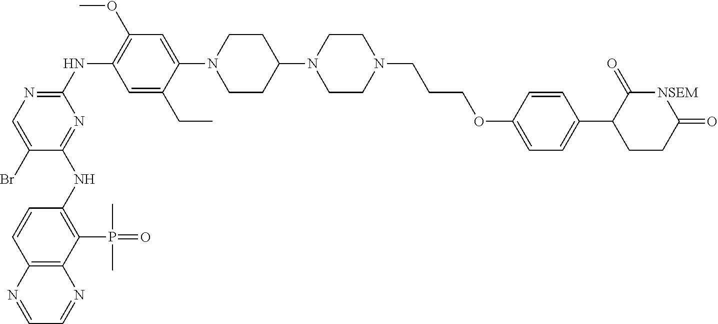 [SMILES: CCc1cc(Nc2ncc(Br)c(Nc3ccc4nccnc4c3P(C)(C)=O)n2)c(OC)cc1N1CCC(N2CCN(CCCOc3ccc(C4CCC(=O)N(COCC[Si](C)(C)C)C4=O)cc3)CC2)CC1]

A mixture of (6-((5-bromo-2-((5-ethyl-2-methoxy-4-(4-(piperazin-1-yl)piperidin-1-yl) phenyl)amino)pyrimidin-4-yl)amino)quinoxalin-5-yl)dimethylphosphine oxide (50 mg, 0.072 mmol), 3-(4-(3-iodopropoxy)phenyl)-1-((2-(trimethylsilyl)ethoxy)methyl)piperidine-2,6-dione (54 mg, 0.11 mmol) and DIEA (28.6 mg, 0.22 mmol) in dichloromethane (3 mL) was stirred in a flask at 85° C. for 16 h. Then the mixture was evaporated in vacuum to afford the 51 mg crude product which was used for next step without further purification. [M+H]⁺=1071.1.

### Step 8: 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propoxy)phenyl)piperidine-2,6-dione

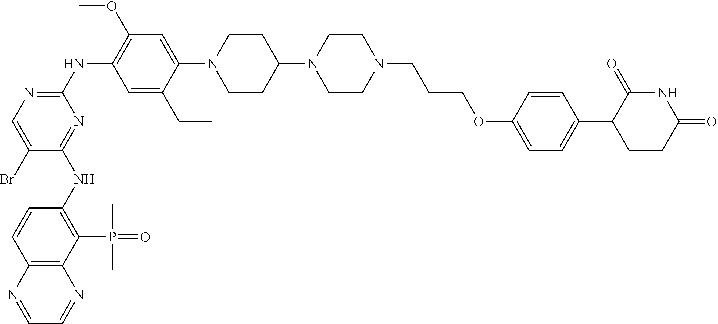 [SMILES: CCc1cc(Nc2ncc(Br)c(Nc3ccc4nccnc4c3P(C)(C)=O)n2)c(OC)cc1N1CCC(N2CCN(CCCOc3ccc(C4CCC(=O)NC4=O)cc3)CC2)CC1]

A mixture of 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propoxy)phenyl)-1-((2-(trimethylsilyl)ethoxy)methyl)piperidine-2,6-dione (51 mg, crude) and trifluoroacetic acid (1 mL) in dichloromethane (2 mL) was stirred in a flask at room temperature overnight. Then the mixture was evaporated in vacuum to afford the crude product, which was dissolved in methanol (2 mL). Ammonium hydroxide (0.5 mL) was added to the resulting solution and stirred for 2 h. Then the mixture was evaporated in vacuum to afford the crude product, which was purified with C-18 column chromatography (0.1% FA in water: acetonitrile=90: 10˜ 60: 40 gradient elution) to give the product to give the product (2.7 mg). ¹H NMR (400 MHz, DMSO) δ 12.65 (s, 1H), 10.81 (s, 1H), 8.86 (d, J=4.3 Hz, 3H), 8.28 (d, J=8.3 Hz, 2H), 7.91 (s, 1H), 7.37 (s, 1H), 7.12 (d, J=8.5 Hz, 2H), 6.89 (d, J=8.4 Hz, 2H), 6.81 (s, 1H), 3.99 (s, 2H), 3.77 (s, 4H), 3.00-3.05 (m, 4H), 2.68-2.70 (m, 6H), 2.37-2.39 (m, 8H), 2.15 (s, 2H), 2.02-2.04 (m, 7H), 1.87 (s, 4H), 1.57 (d, J=11.3 Hz, 2H), 0.93 (s, 3H); [M+H]+=939.1.

### Example 17: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethoxy)phenyl)piperidine-2,6-dione formate

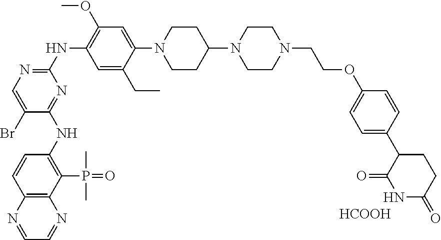 [SMILES: CCc1cc(Nc2ncc(Br)c(Nc3ccc4nccnc4c3P(C)(C)=O)n2)c(OC)cc1N1CCC(N2CCN(CCOc3ccc(C4CCC(=O)NC4=O)cc3)CC2)CC1 ; "HCOOH"]

The titled compound was synthesized in the procedures similar to Example 18.

1H NMR (400 MHz, DMSO) δ 12.65 (s, 1H), 10.81 (s, 1H), 8.85 (t, J=10.0 Hz, 3H), 8.27 (t, J=12.2 Hz, 2H), 7.91 (s, 1H), 7.37 (s, 1H), 7.13 (d, J=8.3 Hz, 2H), 6.91 (d, J=8.3 Hz, 2H), 6.81 (s, 1H), 4.06 (s, 2H), 3.77 (s, 5H), 3.01 (d, J=9.9 Hz, 3H), 2.64 (dd, J=52.0, 22.7 Hz, 10H), 2.31 (s, 2H), 2.17 (d, J=11.5 Hz, 1H), 2.02 (d, J=14.3 Hz, 8H), 1.86 (d, J=10.4 Hz, 2H), 1.57 (d, J=11.3 Hz, 2H), 0.92 (s, 3H); [M+H]⁺=927.

### Example 22: 3-(4-(4-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)piperidin-1-yl)phenyl)piperidine-2,6-dione

### Step 1: (1-(4-bromophenyl)piperidin-4-yl)methanol

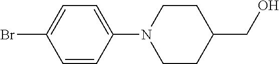 [SMILES: OCC1CCN(c2ccc(Br)cc2)CC1]

1-Bromo-4-iodobenzene (2.0 g, 7.1 mmol), piperidin-4-ylmethanol (894.0 mg, 7.8 mmol), CuI (270.0 mg, 1.4 mmol), L-proline (163.0 mg, 1.4 mmol), K₃PO₄ (3.0 g, 14.2 mmol) were placed in DMSO (20 mL). The resulting mixture was then heated to 80° C. overnight until LC-MS indicated all the starting material was consumed. The mixture was cooled to room temperature, filtered off the solid, diluted with EtOAc (200 mL), and washed with brine for 3 times. The organic layer was dried over Na₂SO₄, and concentrated to afford crude product (2.8 g, crude), which was used directly without further purification. [M+H]⁺=270.0.

### Step 2: (1-(4-(2,6-bis(benzyloxy)pyridin-3-yl)phenyl)piperidin-4-yl)methanol

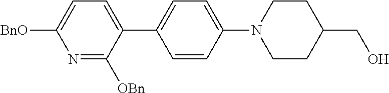 [SMILES: OCC1CCN(c2ccc(-c3ccc(OCc4ccccc4)nc3OCc3ccccc3)cc2)CC1]

(1-(4-Bromophenyl)piperidin-4-yl)methanol (2.8 g, crude), Pd(dppf)Cl₂ (580.0 mg, 0.79 mmol), 2,6-bis(benzyloxy)-3-(4,4,5,5-tetramethyl-1,3,2-dioxaborolan-2-yl)pyridine (3.0 g, 7.1 mmol) and Cs₂CO₃ (4.6 g, 14.2 mmol) were placed in dioxane/water (300 mL, 10:1). The mixture was stirred at 100° C. overnight. The reaction was cooled to room temperature and filtered off the solid. The filtrate was concentrated and purified with SiO₂-gel column (eluted with EtOAc/Hexane=1: 1) to give the desired product (3.0 g, 88%, 2 steps). [M+H]⁺=481.2.

### Step 3: 3-(4-(4-(hydroxymethyl)piperidin-1-yl)phenyl)piperidine-2,6-dione

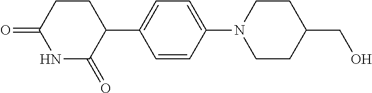 [SMILES: O=C1CCC(c2ccc(N3CCC(CO)CC3)cc2)C(=O)N1]

(1-(4-(2,6-Bis(benzyloxy)pyridin-3-yl)phenyl)piperidin-4-yl)methanol (3.0 g, 6.3 mmol) was dissolved in MeOH (30 mL). Pd/C (10%, w/w, 0.3 g) was added to the solution in one portion. The resulting mixture was stirred under H₂ atmosphere (1 atm) overnight. The solid was filtered off and the filtrate was concentrated to give the crude product. The crude was triturated with MTBE to give the desired product (1.2 g, 63.5%). [M+H]⁺=303.0.

### Step 4: 1-(4-(2,6-dioxopiperidin-3-yl)phenyl)piperidine-4-carbaldehyde

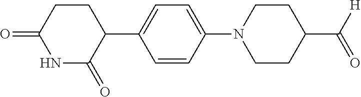 [SMILES: O=CC1CCN(c2ccc(C3CCC(=O)NC3=O)cc2)CC1]

3-(4-(4-(Hydroxymethyl)piperidin-1-yl)phenyl)piperidine-2,6-dione (100.0 mg, 0.33 mmol) was dissolved in DMSO (2 mL). IBX (184.8 mg, 0.66 mmol) was added to the solution in portions at 0° C. and the mixture was stirred at 0° C. for 30 min. Then the mixture was warmed to rt for another 1 h until TLC indicated all the starting material was consumed. The mixture was diluted with water, extracted with EtOAc, washed with brine, dried over Na₂SO₄, and concentrated to give crude the desired product (100.0 mg, crude) which was used directly without further purification.

### Step 5: 3-(4-(4-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)piperidin-1-yl)phenyl)piperidine-2,6-dione

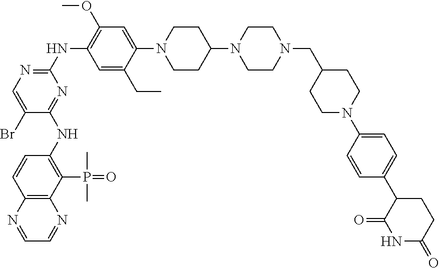 [SMILES: CCc1cc(Nc2ncc(Br)c(Nc3ccc4nccnc4c3P(C)(C)=O)n2)c(OC)cc1N1CCC(N2CCN(CC3CCN(c4ccc(C5CCC(=O)NC5=O)cc4)CC3)CC2)CC1]

The titled compound was synthesized in the procedures similar to that in Example 23 step 12. ¹H NMR (400 MHz, DMSO) δ 12.62 (s, 1H), 10.76 (s, 1H), 8.85 (s, 3H), 8.26 (d, J=11.7 Hz, 2H), 7.89 (s, 1H), 7.35 (s, 1H), 7.00 (s, 2H), 6.87 (s, 2H), 6.79 (s, 1H), 3.68 (d, J=51.3 Hz, 6H), 2.98 (s, 2H), 2.64 (d, J=33.0 Hz, 8H), 2.38-2.20 (m, 5H), 2.13 (s, 3H), 2.00 (d, J=14.3 Hz, 8H), 1.83 (s, 2H), 1.74 (s, 2H), 1.57 (s, 4H), 1.18 (s, 3H), 0.90 (s, 3H); [M+H]⁺=978.

### Example 24: 3-(4-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-2-oxoethyl)piperidin-1-yl)phenyl)piperidine-2,6-dione

### Step 1: 4-(2,6-bis(benzyloxy)pyridin-3-yl) aniline

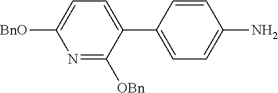 [SMILES: Nc1ccc(-c2ccc(OCc3ccccc3)nc2OCc2ccccc2)cc1]

2,6-bis(benzyloxy)-3-bromopyridine (58.0 g, 156.7 mmol), 4-aminophenylboronic acid pinacol ester (44.6 g, 203.7 mmol), K₂CO₃ (65.0 g, 470.0 mmol) and Pd(dppf)Cl₂ (11.5 g, 15.7 mmol) were added to the reaction flask, the mixture was degassed and purged with nitrogen for three times. Next 1.4-dioxane (1 L) and water (300 mL) were added to the reaction flask, and nitrogen was recharged three times again, the reaction was heated to reflux. After stirring for three hours, the reaction was cooled to room temperature, extracted with EtOAc (500 mL×3), the combined organic phases were washed with water and brine, dried and concentrated. 4-(2,6-bis(benzyloxy)pyridin-3-yl) aniline (57.3 g, 95.6%) was obtained after column separation (petroleum ether: ethyl acetate=10: 1). ¹H NMR (400 MHz, CDCl₃) δ 7.57 (d, J=8.0 Hz, 1H), 7.50-7.27 (m, 12H), 6.72 (d, J=8.5 Hz, 2H), 6.45 (d, J=8.0 Hz, 1H), 5.43 (s, 2H), 5.36 (s, 2H), 3.68 (s, 2H); [M+H]⁺=383.2.

### Step 2: 2,6-bis(benyloxy)-3-(4-iodophenyl)pyridine

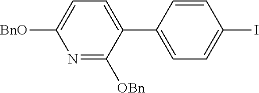 [SMILES: Ic1ccc(-c2ccc(OCc3ccccc3)nc2OCc2ccccc2)cc1]

p-Toluenesulfonic acid monohydrate (106.0 g, 557 mmol) was added to tert-Butanol (800 mL). 4-(2,6-bis(benzyloxy)pyridin-3-yl) aniline (78.5 g, 205 mmol) was dissolved in MeCN (400 mL) and added to the system, and the mixture was stirred at room temperature. NaNO₂ (28.3 g, 404 mmol) and KI (85.2 g, 513.1 mmol) in water (400 mL) was added. Then system was stirred at room temperature. After stirred for 1.5 h, the mixture was diluted with water (1.5 L), and pH was adjusted to 10 with 2M sodium hydroxide solution. 2M sodium thiosulfate solution (1 L) was added to the mixture, extracted with DCM (1L×3). The combined organic phases were washed with water and brine, dried and concentrated. The residue was purified with silica gel column (petroleum ether: ethyl acetate=100: 1) to obtain 2,6-bis(benzyloxy)-3-(4-iodophenyl)pyridine (31.2 g, 30.8%). ¹H NMR (400 MHz, CDCl₃) δ 7.70 (d, J=8.4 Hz, 2H), 7.57 (d, J=8.1 Hz, 1H), 7.47-7.28 (m, 12H), 6.48 (d, J=8.1 Hz, 1H), 5.41 (s, 2H), 5.37 (s, 2H); [M+H]⁺=494.1.

### Step 3: tert-butyl 2-(1-(4-(2,6-bis(benzyloxy)pyridin-3-yl)phenyl)piperidin-4-yl)acetate

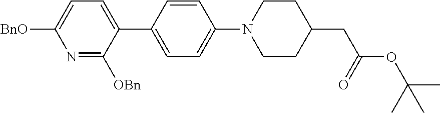 [SMILES: CC(C)(C)OC(=O)CC1CCN(c2ccc(-c3ccc(OCc4ccccc4)nc3OCc3ccccc3)cc2)CC1]

Under the atmosphere of nitrogen, add 2,6-bis(benzyloxy)-3-(4-iodophenyl)pyridine (45.4 g, 92.0 mmol), tert-butyl 2-(piperidin-4-yl)acetate (27.5 g, 138 mmol) and t-BuONa (13.3 g, 138 mmol) to 1.4-dioxane (450 mL). After pumping nitrogen three times, Pd₂(dba)₃ (4.2 g, 4.6 mmol) and X-phos (4.4 g, 9.2 mmol) were added to the system, and then nitrogen was pumped for three times again, then temperature was raised to reflux. After 1.5h, the reaction was cooled to room temperature, water (250 mL) was added, extracted with DCM (3×250 mL). The combined organic phases were washed with water and brine, dried and concentrated. The residue was purified with silica gel column (petroleum ether: ethyl acetate=20: 1) to obtain tert-butyl 2-(1-(4-(2,6-bis(benzyloxy)pyridin-3-yl)phenyl)piperidin-4-yl)acetate (31.1 g, 60.2%). ¹H NMR (400 MHz, CDCl₃) δ 7.59 (d, J=8.1 Hz, 1H), 7.50-7.28 (m, 12H), 6.96 (d, J=8.8 Hz, 2H), 6.46 (d, J=8.0 Hz, 1H), 5.43 (s, 2H), 5.36 (s, 2H), 3.73-3.70 (m, 2H), 2.80-2.74 (m, 2H), 2.21 (d, J=7.0 Hz, 2H), 2.00-1.89 (m, 1H), 1.86-1.82 (m, 2H), 1.53-1.35 (m, 11H). [M+H]⁺=565.3

### Step 4: tert-butyl 2-(1-(4-(2,6-dioxopiperidin-3-yl)phenyl)piperidin-4-yl)acetate

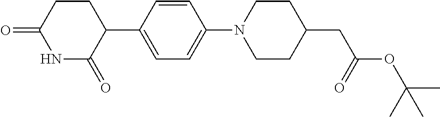 [SMILES: CC(C)(C)OC(=O)CC1CCN(c2ccc(C3CCC(=O)NC3=O)cc2)CC1]

Tert-butyl 2-(1-(4-(2,6-bis(benzyloxy)pyridin-3-yl)phenyl)piperidin-4-yl)acetate (28.6 g, 50.6 mmol) and Pd/C (7.5 g) were added to DMF (500 mL), the mixture was stirred at 50° C. under hydrogen atmosphere for 16 h, cooled to room temperature, filtered through a pad of Celite and washed with DCM. The filtrate was concentrated to get tert-butyl 2-(1-(4-(2,6-dioxopiperidin-3-yl)phenyl)piperidin-4-yl)acetate (17.4 g, 89%). ¹H NMR (400 MHz, CDCl₃) δ 7.97 (s, 1H), 7.07 (d, J=8.5 Hz, 2H), 6.91 (d, J=8.4 Hz, 2H), 3.79-3.49 (m, 3H), 2.82-2.54 (m, 4H), 2.27-2.18 (m, 4H), 1.91-1.87 (m, 1H), 1.83-1.80 (m, 2H), 1.50-1.25 (m, 11H). [M+H]⁺=387.2.

### Step 5: 2-(1-(4-(2.6-dioxopiperidin-3-yl)phenyl)piperidin-4-yl)acetic acid trifluoroacetate

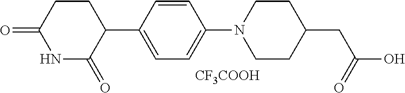

Tert-butyl 2-(1-(4-(2,6-dioxopiperidin-3-yl)phenyl)piperidin-4-yl)acetate (16.2 g, 41.9 mmol) and TFA (95.5 g, 838 mmol) were added to DCM (100 mL). The temperature was raised to 40° C. and stirred for 1.5 h. After cooling to room temperature, the mixture was concentrated, then recrystallized in MTBE (150 mL) to give the product (14.5 g, 77.9%). ¹H NMR (400 MHz, MeOD) δ 7.57 (d, J=8.6 Hz, 2H), 7.47 (d, J=8.6 Hz, 2H), 3.96 (dd, J=11.7, 5.0 Hz, 1H), 3.71-3.68 (m, 2H), 3.61-3.56 (m, 2H), 2.78-2.63 (m, 2H), 2.39-2.38 (m, 2H), 2.27-2.13 (m, 6H), 1.79-1.69 (m, 2H). [M+H]⁺=331.2.

### Step 6: 3-(4-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-2-oxoethyl)piperidin-1-yl)phenyl)piperidine-2,6-dione

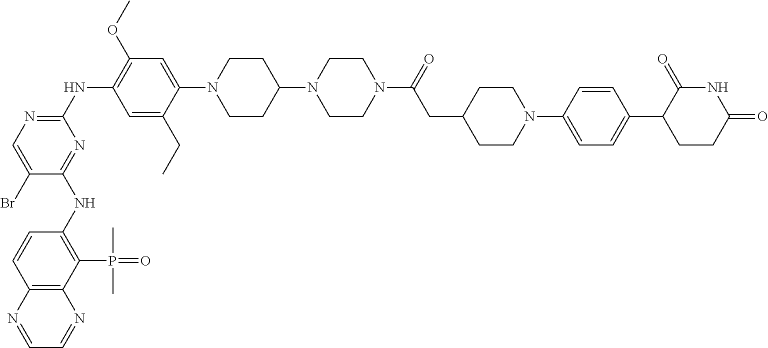 [SMILES: CCc1cc(Nc2ncc(Br)c(Nc3ccc4nccnc4c3P(C)(C)=O)n2)c(OC)cc1N1CCC(N2CCN(C(=O)CC3CCN(c4ccc(C5CCC(=O)NC5=O)cc4)CC3)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (40 mg, 0.057 mmol) and 2-(1-(4-(2,6-dioxopiperidin-3-yl)phenyl)piperidin-4-yl)acetic acid trifluoroacetate (37 mg, 0.083 mmol), HATU (26.2 mg, 0.069 mmol) and DIEA (14.7 mg, 0.114 mmol) in DMF (4 mL) was stirred in a flask at room temperature for 2 hours. The reaction was quenched with water and the mixture was extracted with DCM. The organic phase was washed three times with saturated brine, and dried over anhydrous Na₂SO₄. After filtration, the filtrate was concentrated under reduced pressure. The residue was purified by HPLC chromatography to give the product (10 mg, 17%). ¹H NMR (400 MHz, DMSO) δ 12.65 (s, 1H), 10.78 (s, 1H), 8.86 (d, J=5.4 Hz, 3H), 8.28 (d, J=7.6 Hz, 2H), 7.90 (d, J=9.9 Hz, 1H), 7.37 (s, 1H), 7.03 (d, J=7.8 Hz, 2H), 6.88 (d, J=8.5 Hz, 2H), 6.81 (s, 1H), 3.77 (s, 3H), 3.64 (s, 3H), 3.48 (s, 4H), 3.02 (d, J=9.2 Hz, 3H), 2.66 (dd, J=26.9, 13.5 Hz, 7H), 2.29 (d, J=6.3 Hz, 7H), 2.02 (d, J=14.2 Hz, 7H), 1.84 (s, 3H), 1.74 (s, 2H), 1.60 (d, J=9.6 Hz, 2H), 1.36-1.19 (m, 3H), 0.93 (s, 3H); [M+H]⁺=1006.4.

### Example 47: 3-((4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propoxy)phenyl)amino)piperidine-2,6-dione

### Step 1: 3-(4-nitronphenoxy)propan-1-ol

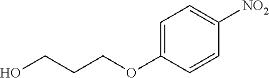 [SMILES: O=[N+]([O-])c1ccc(OCCCO)cc1]

To a solution of 4-nitrophenol (50.0 g, 359 mmol) in DMF (250 mL) was added Cs₂CO₃ (351 g, 1.08 mol), KI (17.9 g, 108 mmol) and 3-chloropropan-1-ol (35.7 g, 377 mmol), the mixture was stirred at 110° C. for 12 hrs. The mixture was partitioned between ethyl acetate (400 mL×2) and H₂O (1500 mL). The combined organic layers were washed with water, dried over Na₂SO₄ and evaporated to dryness. The residue was purified by column chromatography (SiO₂, Petroleum ether: Ethyl acetate=1: 0 to 0: 1) to afford the product (70.0 g, 32.9%). [M+H]⁺=198.

### Step 2: 3-(4-aminophenoxy)propan-1-ol

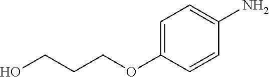 [SMILES: Nc1ccc(OCCCO)cc1]

To a solution of 3-(4-nitrophenoxy)propan-1-ol (70.0 g, 355 mmol) in MeOH (350 mL) was added Pd/C (7.00 g, 10.0% purity) under N₂. The suspension was degassed under vacuum and purged with H₂ several times. The mixture was stirred under H₂ (50 psi) at 50° C. for 2 hrs. The suspension was filtered through a pad of celite and the filter cake was washed with MeOH (100 mL×3) to give the product (30.0 g, 50.5%). ¹H NMR (CD₃OD, 400 MHz) δ 6.63-6.78 (m, 4H), 3.98 (t, J=6.17 Hz, 2H), 3.72 (t, J=6.28 Hz, 2H), 1.93 (quin, J=6.34 Hz, 2H); [M+H]⁺=168.

### Step 3: 3-((4-(3-hydroxypropoxy)phenyl)amino)piperidine-2,6-dione

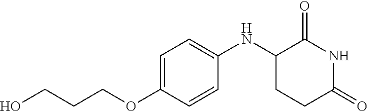 [SMILES: O=C1CCC(Nc2ccc(OCCCO)cc2)C(=O)N1]

To a solution of 3-(4-aminophenoxy)propan-1-ol (25.0 g, 150 mmol), 3-bromopiperidine-2,6-dione (30.1 g, 157 mmol) in DMF (150 mL) was added DIEA (38.7 g, 299 mmol), then the mixture was stirred at 65° C. for 12 hrs. The mixture was partitioned between ethyl acetate (200 mL) and H₂O (450 mL). The separated organic layer was dried over Na₂SO₄ and evaporated to dryness. The residue was purified by column chromatography to afford the product (25.2 g, 59.5%). ¹H NMR (CD₃OD, 400 MHz) δ 6.75-6.82 (m, 2H), 6.67-6.75 (m, 2H), 4.16 (dd, J=11.8, 4.83 Hz, 1H), 3.99 (t, J=6.24 Hz, 2H), 3.73 (t, J=6.36 Hz, 2H), 2.63-2.86 (m, 2H), 2.32 (dtd, J=13.3, 5.01, 5.01, 3.61 Hz, 1H), 1.79-2.02 (m, 3H); [M+H]⁺=279.

### Step 4: 3-(4-((2,6-dioxopiperidin-3-yl)amino)phenoxy)propyl methanesulfonate

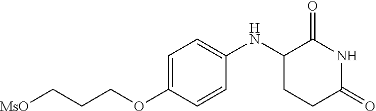 [SMILES: CS(=O)(=O)OCCCOc1ccc(NC2CCC(=O)NC2=O)cc1]

To a solution of 3-((4-(3-hydroxypropoxy)phenyl)amino)piperidine-2,6-dione (1.0 g, 3.6 mmol) in DCM (250 mL) was added DIEA (0.93 g, 7.2 mmol). The resulting reaction mixture was cool down to 0° C., then MsCl (0.62 g, 5.4 mmol) was added. The mixture was stirred at 25° C. for 2 hrs. The resulting mixture was partitioned between ethyl acetate (100 mL×2) and H₂O (150 mL). The combined organic layers were washed with water, dried over Na₂SO₄ and evaporated to dryness. The residue was purified by column chromatography to afford the product (0.3 g, 23%). [M+H]⁺=357.

### Step 5: 3-((4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propoxy)phenyl)amino)piperidine-2,6-dione

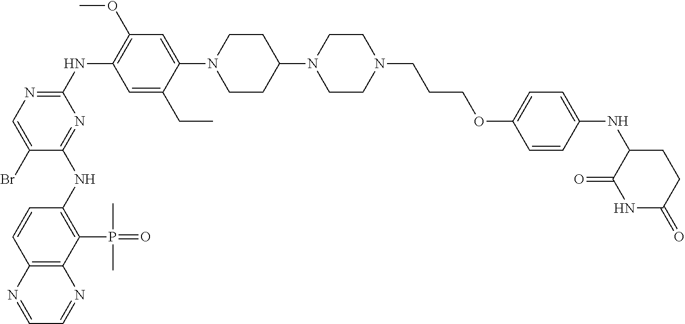 [SMILES: CCc1cc(Nc2ncc(Br)c(Nc3ccc4nccnc4c3P(C)(C)=O)n2)c(OC)cc1N1CCC(N2CCN(CCCOc3ccc(NC4CCC(=O)NC4=O)cc3)CC2)CC1]

The titled compound was prepared in a manner similar to that in Example 13 step 5.

¹H NMR (400 MHz, DMSO) δ 12.64 (s, 1H), 10.77 (s, 1H), 8.86 (s, 3H), 8.35 (s, 1H), 8.27 (s, 2H), 7.91 (s, 1H), 7.37 (s, 1H), 6.81 (s, 1H), 6.71 (s, 2H), 6.63 (s, 2H), 5.42 (s, 1H), 4.19 (s, 1H), 3.88 (s, 2H), 3.77 (s, 3H), 3.00 (s, 6H), 2.69 (d, J=12.1 Hz, 4H), 2.36-2.38 (m, 7H), 2.02-2.06 (m, 8H), 1.84 (m, 5H), 1.58 (m, 2H), 0.92 (s, 3H); [M+H]⁺=954.3.

### Example 48: 3-((4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)phenyl)amino)piperidine-2,6-dione

### Step 1: 4-(2-((tert-butyldimethylsilyl)oxy)ethyl) aniline

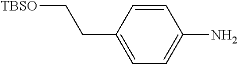 [SMILES: CC(C)(C)[Si](C)(C)OCCc1ccc(N)cc1]

2-(4-aminophenyl)ethan-1-ol (13.7 g, 100 mmol), TEA (20.0 g, 200 mmol) and DMAP (1.2 g, 10 mmol) were placed in DCM (150 mL). TBSCl (17.0 g, 110 mmol) was added to the solution in dropwise at 0° C. The resulting mixture was stirred at room temperature for 1h. The mixture was diluted with ice-water, extracted with DCM, the combined organic phases were washed with 0.5 M HCl solution for 3 times and Brine for once. The resulting organic phase was dried over Na₂SO₄, concentrated to give the desired product (16.9 g, 67.3%), which was used directly without further purification. [M+H]⁺=252.2

### Step 2: 3-((4-(2-((tert-butyldimethylsilyl)oxy)ethyl)phenyl)amino)piperidine-2,6-dione

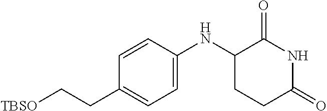 [SMILES: CC(C)(C)[Si](C)(C)OCCc1ccc(NC2CCC(=O)NC2=O)cc1]

4-(2-((tert-butyldimethylsilyl)oxy)ethyl) aniline (9.4 g, 37.5 mmol), 3-bromopiperidine-2,6-dione (10.8 g, 56 mmol) and DIEA (9.7 g, 75 mmol) were placed in MeCN (200 mL). The mixture was stirred at 80° C. for 8h. Cooling the reaction to room temperature, concentrated and purified with SiO₂-gel column to give the desired product (4.8 g, 35.2%). [M+H]⁺=363.2.

### Step 3: 3-((4-(2-hydroxyethyl)phenyl)amino)piperidine-2,6-dione hydrochloride

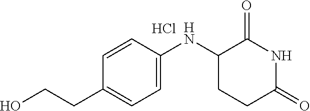 [SMILES: O=C1CCC(Nc2ccc(CCO)cc2)C(=O)N1 ; Cl]

3-((4-(2-((tert-butyldimethylsilyl)oxy)ethyl)phenyl)amino)piperidine-2,6-dione (4.8 g, 13.2 mmol) was placed in HCl-dixoane (4M, 30 mL), the mixture was stirred at room temperature for 2h. Concentrated and triturated with MTBE to afford desired product (3.1 g, 95%). [M+H]⁺=249.1.

### Step 4: 4-((2,6-dioxopiperidin-3-yl)amino)phenethyl 4-methylbenzenesulfonate

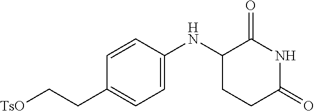 [SMILES: Cc1ccc(S(=O)(=O)OCCc2ccc(NC3CCC(=O)NC3=O)cc2)cc1]

Into a 25-mL flask, 3-((4-(2-hydroxyethyl)phenyl)amino)piperidine-2,6-dione hydrochloride was placed (170.0 mg, 0.6 mmol) was dissolved in pyridine (4.0 mL), TsCl (230.0 mg, 1.2 mmol) was added. The resulting solution was stirred overnight at room temperature. The resulting mixture was concentrated under vacuum. The residue was applied onto a silica gel column with dichloromethane/methanol (7:1) to afford the product (100 mg, 41.7%). [M+H]⁺=403.

### Step 5: 3-((4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)phenyl)amino)piperidine-2,6-dione

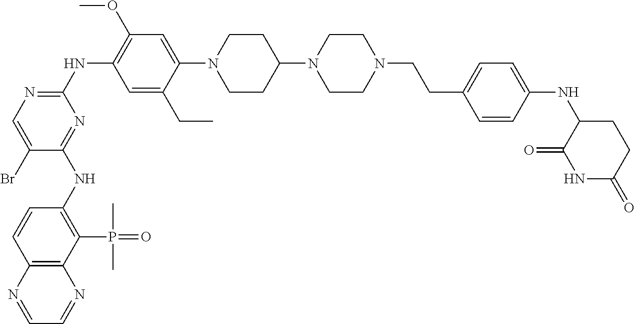 [SMILES: CCc1cc(Nc2ncc(Br)c(Nc3ccc4nccnc4c3P(C)(C)=O)n2)c(OC)cc1N1CCC(N2CCN(CCc3ccc(NC4CCC(=O)NC4=O)cc3)CC2)CC1]

The titled compound was prepared in a manner similar to that in Example 13 step 5. ¹H NMR (400 MHz, DMSO) δ 12.63 (s, 1H), 10.77 (s, 1H), 8.85 (d, J=4.4 Hz, 3H), 7.89 (s, 1H), 7.35 (s, 1H), 6.92 (d, J=8.3 Hz, 2H), 6.79 (s, 1H), 6.58 (d, J=8.3 Hz, 2H), 5.64 (d, J=7.6 Hz, 1H), 4.25 (s, 1H), 3.75 (s, 3H), 2.99 (d, J=9.5 Hz, 3H), 2.80-2.62 (m, 4H), 2.50-2.60 (m, 7H), 2.20-2.39 (m, 7H), 2.00 (d, J=14.3 Hz, 8H), 1.83 (s, 4H), 1.48-1.60 (m, 2H), 0.91 (s, 3H); [M+H]⁺=924.

### Example 79: 1-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)phenyl)dihydropyrimidine-2,4(1H,3H)-dione

### Step 1: (1-(4-nitrophenyl)piperidin-4-yl)methanol

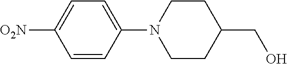 [SMILES: O=[N+]([O-])c1ccc(N2CCC(CO)CC2)cc1]

To a solution of 1-fluoro-4-nitrobenzene (100 g, 710 mmol) and 4-piperidinemethanol (98 g, 85.0 mmol) in DMF (1400 mL) was added K₂CO₃ (196 g) at 25° C. The mixture reaction was stirred at 80° C. for 15 h. The reaction was cooled to room temperature, poured into ice-water (6000 mL) and stirred for 20 mins. The solid was filtered and washed with water (500 mL×2), dried to give the product (140 g, 83.8%). ¹H NMR (400 MHz, DMSO) δ_{H} 8.03 (d, J=9.4 Hz, 2H), 7.01-6.98 (m, 2H), 4.54 (t, J=5.3 Hz, 1H), 4.07-4.04 (m, 2H), 3.29-3.26 (m, 2H), 3.00-2.93 (m, 2H), 1.76-1.67 (m, 3H), 1.21-1.11 (m, 2H); [M+H]⁺=237.2.

### Step 2: (1-(4-aminophenyl)piperidin-4-yl)methanol

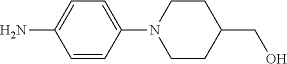 [SMILES: Nc1ccc(N2CCC(CO)CC2)cc1]

Under N₂, to a solution of (1-(4-nitrophenyl)piperidin-4-yl)methanol (140 g, 592 mmol) in MeOH (1680 mL) was added 10% Pd/C (28 g) at 25° C. And then the mixture was exchanged with H₂ two times and stirred under H₂ atmosphere at 25° C. for 15 h. The mixture was filtered through a pad of Celite and washed with MeOH (140.0 mL). The filtrate was concentrated under vacuum to obtain the product (113 g, 92.0%). ¹H NMR (400 MHz, DMSO) δ_{H} 6.77-6.61 (m, 2H), 6.54-6.38 (m, 2H), 4.53 (brs, 2H), 4.45 (t, J=5.3 Hz, 1H), 3.32-3.27 (m, 2H), 2.46-2.41 (m, 2H), 1.76-1.62 (m, 2H), 1.50-1.31 (m, 1H), 1.27-1.08 (m, 2H); [M+H]⁺=207.2.

### Step 3: (1-(4-(2,4-dioxotetrahydropyrimidin-1(2H)-yl)phenyl)piperidin-4-yl)methyl acetate

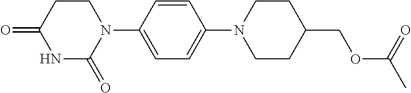 [SMILES: CC(=O)OCC1CCN(c2ccc(N3CCC(=O)NC3=O)cc2)CC1]

To a solution of (1-(4-aminophenyl)piperidin-4-yl)methanol (25 g, 121 mmol) in PhMe (183 mL) was added acrylic acid (13 g, 180 mmol) at 25° C. The mixture was stirred at 90° C. for 15 h. The reaction was cooled to 25° C., then AcOH (183 mL) and urea (36.4 g, 606 mmol) were added. The mixture was stirred at 110° C. for 24 h. The reaction was cooled to 25° C. and concentrated under vacuum. The residue was dissolved with EtOAc (500 mL) and then adjusted to pH=7 with sat. NaHCO₃. The resulting solution was extracted with 2×200 mL of EtOAc and the organic layers were combined. The organic layer was dried over anhydrous sodium sulfate and concentrated under vacuum, and the residue was purified on silica gel to give the product (17.5 g, 42%). ¹H NMR (400 MHz, DMSO) δ_{H} 10.32 (s, 1H), 7.20 (d, J=8.9 Hz, 2H), 6.99 (d, J=9.0 Hz, 2H), 3.98 (d, J=6.2 Hz, 2H), 3.80-3.66 (m, 4H), 2.74-2.72 (m, 4H), 2.09 (s, 3H), 1.80 (d, J=13.8 Hz, 4H), 1.37 (dd, J=12.1, 2.8 Hz, 3H); [M+H]⁺=346.2.

### Step 4: 1-(4-(4-(hydroxymethyl)piperidin-1-yl)phenyl)dihydropyrimidine-2,4(1H,3H)-dione

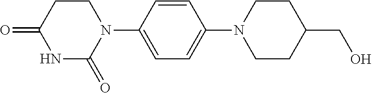 [SMILES: O=C1CCN(c2ccc(N3CCC(CO)CC3)cc2)C(=O)N1]

(1-(4-(2,4-Dioxotetrahydropyrimidin-1(2H)-yl)phenyl)piperidin-4-yl)methyl acetate (35.0 g, 101 mmol) was added into 2N HCl (260.0 mL) at 25° C. The mixture was stirred at 100° C. for 15 h. Reaction was monitored by HPLC. The reaction was cooled to 10° C. and then adjusted to pH=7 with sat. NaHCO₃. The solid was collected by filtrated, washed by water (50.0 mL), and dried to obtain the product (16.9 g, 55%). ¹H NMR (400 MHz, DMSO) δ_{H} 10.26 (s, 1H), 7.13 (d, J=8.9 Hz, 2H), 6.92 (d, J=9.0 Hz, 2H), 4.49 (s, 1H), 3.78-3.61 (m, 4H), 3.30-3.28 (m, 2H), 2.70-2.66 (m, 4H), 1.75-1.72 (m, 2H), 1.52-1.49 (m, 1H), 1.28-1.18 (m, 2H); [M+H]⁺=304.2.

### Step 5: 1-(4-(2,4-dioxotetrahydropyrimidin-1(2H)-yl)phenyl)piperidine-4-carbaldehyde

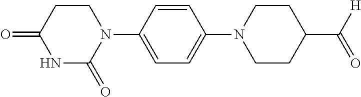 [SMILES: O=CC1CCN(c2ccc(N3CCC(=O)NC3=O)cc2)CC1]

To a solution of 1-(4-(4-(hydroxymethyl)piperidin-1-yl)phenyl)dihydropyrimidine-2,4(1H,3H)-dione (15.0 g, 49 mmol) in DMSO (120 mL) was added IBX (32.7 g, 117 mmol) in portions at 25° C. The mixture was stirred at 25° C. for 15 h. Water (300 mL) was added to the reaction at 25° C. The solid was filtered and washed with water (100 mL), EtOAc (100 mL). The resulting solution was extracted with 4×200 mL of EtOAc. The combined organic layers were dried over Na₂SO₄ and concentrated under vacuum to afford a crude residue. The crude product was purified by column chromatography to afford the product (3.1 g, 22.1%). ¹H NMR (300 MHz, DMSO) δ_{H} 10.26 (s, 1H), 9.63 (s, 1H), 7.15-7.10 (m, 2H), 6.95-6.89 (m, 2H), 3.71-3.51 (m, 4H), 2.86-2.57 (m, 4H), 1.94-1.91 (m, 1H), 1.77-1.73 (m, 1H), 1.64-1.51 (m, 2H), 1.38-1.30 (m, 1H).

### Step 6: 1-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)phenyl)dihydropyrimidine-2.4(1H,3H)-dione

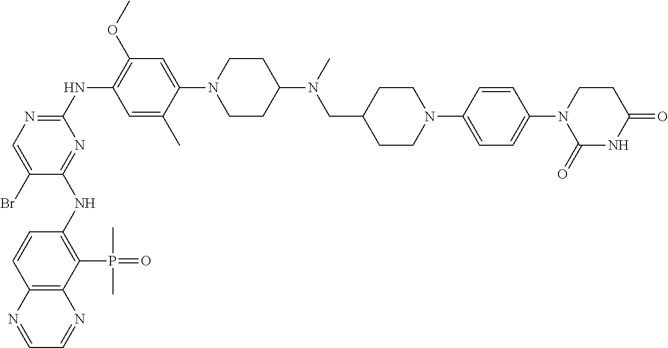 [SMILES: COc1cc(N2CCC(N(C)CC3CCN(c4ccc(N5CCC(=O)NC5=O)cc4)CC3)CC2)c(C)cc1Nc1ncc(Br)c(Nc2ccc3nccnc3c2P(C)(C)=O)n1]

The titled compound was prepared in a manner similar to that in Example 23 step 12.

¹H NMR (400 MHz, DMSO) δ 12.69 (s, 1H), 10.26 (s, 1H), 8.86 (d, J=8.0 Hz, 3H), 8.27 (s, 2H), 7.93 (d, J=8.8 Hz, 1H), 7.36 (s, 1H), 7.13 (d, J=8.7 Hz, 2H), 6.93 (d, J=8.8 Hz, 2H), 6.76 (s, 1H), 3.77 (s, 3H), 3.69 (s, 4H), 3.09 (s, 2H), 2.68 (d, J=10.0 Hz, 6H), 2.30 (m, 5H), 2.10 (s, 3H), 2.02 (d, J=14.3 Hz, 6H), 1.80 (s, 4H), 1.63 (d, J=9.9 Hz, 3H), 1.22 (d, J=11.9 Hz, 2H); [M+H]⁺=910.

### Example 55: 1-(4-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)piperidin-1-yl)phenyl)dihydropyrimidine-2,4(1H,3H)-dione

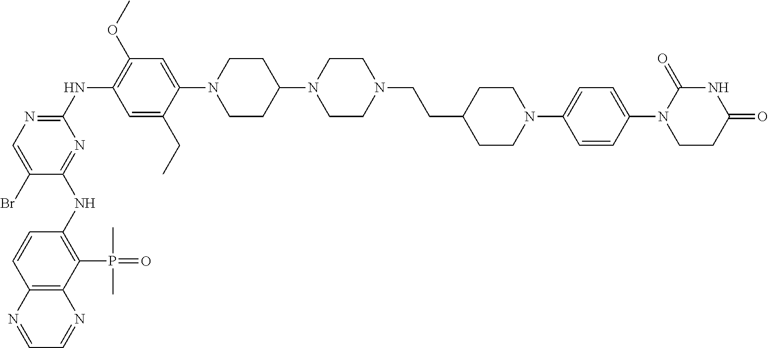 [SMILES: CCc1cc(Nc2ncc(Br)c(Nc3ccc4nccnc4c3P(C)(C)=O)n2)c(OC)cc1N1CCC(N2CCN(CCC3CCN(c4ccc(N5CCC(=O)NC5=O)cc4)CC3)CC2)CC1]

The titled compound was synthesized in the procedures similar to Example 79. ¹H NMR (400 MHz, DMSO) δ 12.65 (s, 1H), 10.26 (s, 1H), 8.86 (d, J=4.5 Hz, 3H), 8.27 (s, 2H), 7.91 (s, 1H), 7.12 (s, 2H), 6.93 (s, 2H), 6.81 (s, 1H), 3.77 (s, 3H), 3.68 (d, J=6.4 Hz, 4H), 3.00 (s, 3H), 2.69 (d, J=7.3 Hz, 8H), 2.28-2.35 (m, 10H), 2.02 (d, J=14.4 Hz, 6H), 1.85 (s, 2H), 1.75 (d, J=11.9 Hz, 2H), 1.57 (d, J=9.7 Hz, 2H), 1.41 (s, 3H), 1.25 (d, J=9.8 Hz, 3H), 0.92 (s, 3H); [M+H]⁺=993.5.

### Example 56: 5-(3-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propyl)azetidin-1-yl)-2-(2,6-dioxopiperidin-3-yl)isoindoline-1,3-dione

### Step 1: 3-(azetidin-3-yl)propan-1-ol

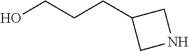

Into a 25-mL flask, was placed tert-butyl 3-(3-hydroxypropyl) azetidine-1-carboxylate (950 mg, 4.413 mmol), DCM (4.0 mL), TFA (2.0 mL, 2.693 mmol). The resulting solution was stirred for 1 hour at room temperature. The resulting mixture was concentrated under vacuum to afford 1.4 g crude product. [M+H]⁺=116.

### Step 2: 2-(2,6-dioxopiperidin-3-yl)-5-(3-(3-hydroxypropyl)azetidin-1-yl)isoindoline-1,3-dione

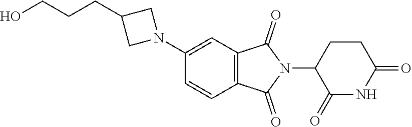 [SMILES: O=C1CCC(N2C(=O)c3ccc(N4CC(CCCO)C4)cc3C2=O)C(=O)N1]

Into a 50-mL flask, was placed 3-(azetidin-3-yl)propan-1-ol (1.40 g, crude), DMSO (10 mL), 2-(2,6-dioxopiperidin-3-yl)-5-fluoroisoindole-1,3-dione (1.21 g, 4.38 mmol), DIEA (2.83 g, 21.9 mmol). The resulting solution was stirred for 1 hour at 80° C. The reaction mixture was cooled to room temperature. The resulting solution was diluted with EA. The resulting solution was extracted with H₂O and the organic layers were combined and dried over anhydrous sodium sulfate and concentrated under vacuum. The residue was applied onto a silica gel column with dichloromethane/methanol (8:1) to afford the product (550 mg, 33.6% for two steps). [M+H]⁺=372.

### Step 3: 3-(1-(2-(2,6-dioxopiperidin-3-yl)-1,3-dioxoisoindolin-5-yl)azetidin-3-yl)propyl 4-methylbenzenesulfonate

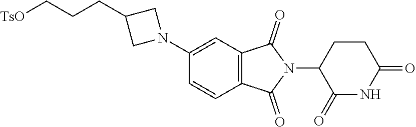 [SMILES: Cc1ccc(S(=O)(=O)OCCCC2CN(c3ccc4c(c3)C(=O)N(C3CCC(=O)NC3=O)C4=O)C2)cc1]

Into a 25-mL flask, was placed 2-(2,6-dioxopiperidin-3-yl)-5-(3-(3-hydroxypropyl)azetidin-1-yl)isoindoline-1,3-dione (480 mg, 1.29 mmol), DCM (10 mL), TEA (262 mg, 2.59 mmol), TsCl (493 mg, 2.59 mmol). The resulting solution was stirred overnight at room temperature. The resulting mixture was concentrated under vacuum. The residue was applied onto a silica gel column with dichloromethane/methanol (7:1) to afford the product (400 mg, 58.89%).[M+H]⁺=526.

### Step 4: 5-(3-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propyl)azetidin-1-yl)-2-(2,6-dioxopiperidin-3-yl)isoindoline-1,3-dione

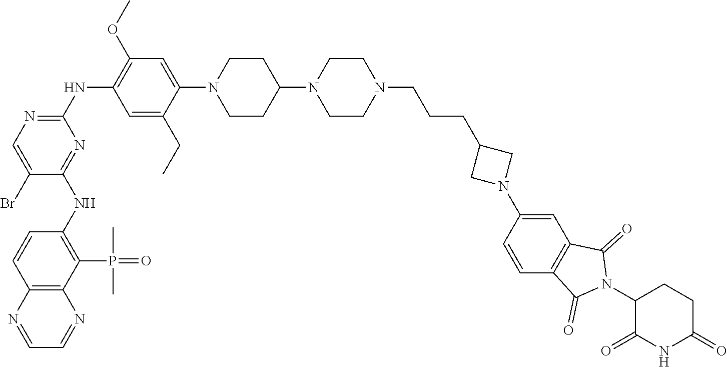 [SMILES: CCc1cc(Nc2ncc(Br)c(Nc3ccc4nccnc4c3P(C)(C)=O)n2)c(OC)cc1N1CCC(N2CCN(CCCC3CN(c4ccc5c(c4)C(=O)N(C4CCC(=O)NC4=O)C5=O)C3)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (40 mg, 0.057 mmol), 3-(1-(2-(2,6-dioxopiperidin-3-yl)-1,3-dioxoisoindolin-5-yl)azetidin-3-yl)propyl 4-methylbenzenesulfonate (37 mg, 0.069 mmol), KI (11.6 mg, 0.069 mmol) and DIEA (14.7 mg, 0.114 mmol) in acetonitrile (4 mL) was stirred in a flask at 75° C. for 12 hours. The reaction was quenched with water and the mixture was extracted with DCM, washed with saturated brine, dried over anhydrous Na₂SO₄. After filtration, the filtrate was concentrated under reduced pressure, The residue was purified by HPLC chromatography (0.1% FA in water: acetonitrile=90:10˜ 50:50 gradient elution) to give the product (10 mg, 17%). ¹H NMR (400 MHz, DMSO) δ 12.65 (s, 1H), 11.08 (s, 1H), 8.86 (d, J=6.5 Hz, 3H), 8.28 (d, J=7.4 Hz, 2H), 7.91 (s, 1H), 7.64 (d, J=8.3 Hz, 1H), 7.37 (s, 1H), 6.81 (s, 1H), 6.76 (s, 1H), 6.63 (d, J=8.0 Hz, 1H), 5.06 (d, J=7.5 Hz, 1H), 4.13 (d, J=8.4 Hz, 2H), 3.77 (s, 3H), 3.66 (s, 2H), 3.02 (d, J=10.0 Hz, 3H), 2.85-2.89 (m, 2H), 2.80-2.56 (m, 8H), 2.90-2.33 (m, 3H), 2.02 (d, J=14.1 Hz, 8H), 1.87 (s, 2H), 1.60-1.65 (m, 4H), 1.46 (s, 2H), 1.23 (s, 3H), 0.92 (s, 3H); [M+H]⁺=1047.5.

### Example 57: 5-(3-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-3-oxopropyl)azetidin-1-yl)-2-(2,6-dioxopiperidin-3-yl)isoindoline-1,3-dione

### Step 1: tert-butyl(E)-3-(3-ethoxy-3-oxoprop-1-en-1-yl)azetidine-1-carboxylate

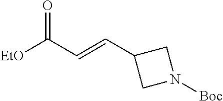 [SMILES: CCOC(=O)/C=C/C1CN(C(=O)OC(C)(C)C)C1]

To a solution of tert-butyl 3-formylazetidine-1-carboxylate (700 mg, 3.78 mmol) in DCM (20 mL) was added Ethyl (triphenylphosphoranylidene)acetate (1.44 g, 4.158 mmol) at 0° C., Then the mixture was stirred at 25° C. overnight. The reaction was quenched with water and extracted with DCM (2×20 mL). The combined organic layers were washed with brine, dried over anhydrous Na₂SO₄, and evaporated in vacuum to afford the crude product, which was further purified with silica gel column chromatography (DCM: MeOH=100: 0˜20: 1 gradient elution) to give the product (640 mg, 66%). [M+Na]⁺=278.1.

### Step 2: tert-butyl 3-(3-ethoxy-3-oxopropyl)azetidine-1-carboxylate

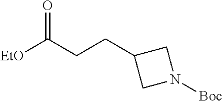 [SMILES: CCOC(=O)CCC1CN(C(=O)OC(C)(C)C)C1]

Under N₂, to a solution of tert-butyl(E)-3-(3-ethoxy-3-oxoprop-1-en-1-yl)azetidine-1-carboxylate (640 mg, 2.51 mmol) in MeOH (10 mL) was added 10% Pd/C (150 mg) at 25° C. And then the mixture was exchanged with H₂ two times and stirred under H₂ atmosphere at 25° C. for 15 h. The mixture was filtered through a pad of Celite and washed with MeOH (15 mL). The filtrate was concentrated under vacuum to obtain the product (580 mg, 90%). [M+Na]⁺=280.1.

### Step 3: 3-(1-(tert-butoxycarbonyl)azetidin-3-yl)propanoic acid

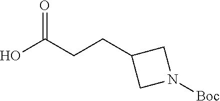 [SMILES: CC(C)(C)OC(=O)N1CC(CCC(=O)O)C1]

To a solution of tert-butyl 3-(3-ethoxy-3-oxopropyl)azetidine-1-carboxylate (580 mg, 2.26 mmol) in THF (10 mL) and H₂O (2 mL) was added lithium hydroxide hydrate (190 mg, 4.52 mmol) at 25° C. The resulting mixture was stirred at 25° C. for 12 h. The reaction was quenched with HCl (1 N) at 0° C. until pH=5 and extracted with DCM (2×40 mL). The combined organic layers were washed with brine, dried over anhydrous Na₂SO₄, and evaporated under vacuum to afford the crude product (430 mg, 83%), which was used for next step without further purification. [M+Na]⁺=252.2.

### Step 4: 3-(azetidin-3-yl)propanoic acid trifluoroacetate

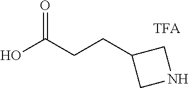

A solution of 3-(1-(tert-butoxycarbonyl)azetidin-3-yl)propanoic acid (430 mg, 1.894 mmol) in DCM (10 mL) was added TFA (1 mL). The mixture was stirred in a flask at room temperature for 3 h. The mixture was evaporated in vacuum to afford the crude product (220 mg, 51%), which was used for next step without further purification. [M+H]⁺=130.1.

### Step 5: 3-(1-(2-(2,6-dioxopiperidin-3-yl)-1,3-dioxoisoindolin-5-yl)azetidin-3-yl)propanoic acid

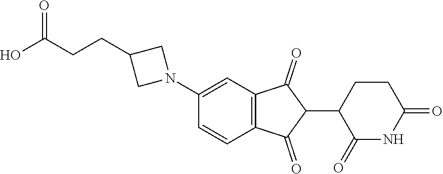 [SMILES: O=C(O)CCC1CN(c2ccc3c(c2)C(=O)C(C2CCC(=O)NC2=O)C3=O)C1]

Into a 100-mL flask, was placed 3-(azetidin-3-yl)propanoic acid trifluoroacetate (220 mg, 0.969 mmol), DMSO (6 mL), 2-(2,6-dioxopiperidin-3-yl)-5-fluoroisoindole-1,3-dione (295 mg, 1.066 mmol), DIEA (625 mg, 4.845 mmol). The resulting solution was stirred for 3 hours at 80° C. The reaction mixture was cooled to room temperature and HCl (1 N) was added until pH ˜ 6. Water was added to the mixture and extracted with DCM (2×30 mL). The combined organic layers were washed with brine, dried over anhydrous Na₂SO₄, and evaporated in vacuum to afford the crude product, which was further purified with silica gel column chromatography (DCM: MeOH=100: 0˜85: 15 gradient elution) to give the product (65 mg, 17%). [M+H]⁺=386.1.

### Step 6: 5-(3-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-3-oxopropyl)azetidin-1-yl)-2-(2,6-dioxopiperidin-3-yl)isoindoline-1,3-dione

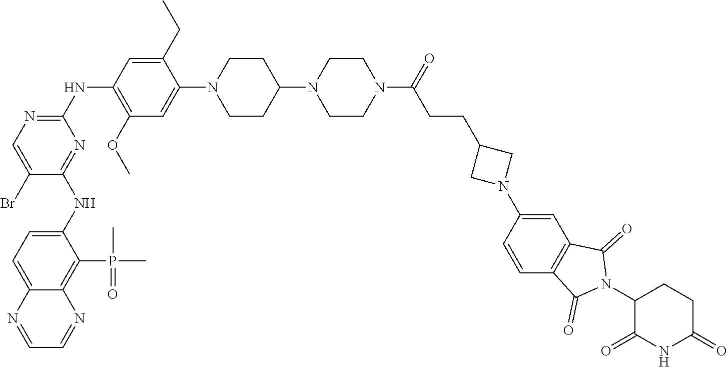 [SMILES: CCc1cc(Nc2ncc(Br)c(Nc3ccc4nccnc4c3P(C)(C)=O)n2)c(OC)cc1N1CCC(N2CCN(C(=O)CCC3CN(c4ccc5c(c4)C(=O)N(C4CCC(=O)NC4=O)C5=O)C3)CC2)CC1]

To a solution of 3-(1-(2-(2,6-dioxopiperidin-3-yl)-1,3-dioxoisoindolin-5-yl)azetidin-3-yl)propanoic acid (40 mg, 0.104 mmol), HATU (44 mg, 0.114 mmol) and DIEA (67 mg, 0.52 mmol) in DMF (5 mL) was added (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (72 mg, 0.104 mmol). The resulting mixture was stirred at room temperature for 3 h. The reaction was directly purified with pre-HPLC (0.1% FA in water: acetonitrile=90:10˜ 60:40 gradient elution) to give the product (22 mg, 20%). ¹H NMR (400 MHz, DMSO) δ 12.65 (s, 1H), 11.06 (s, 1H), 8.86 (d, J=6.3 Hz, 3H), 8.27 (s, 2H), 7.90 (d, J=10.4 Hz, 1H), 7.64 (d, J=8.2 Hz, 1H), 7.38 (s, 1H), 6.81 (s, 1H), 6.75 (s, 1H), 6.62 (d, J=8.3 Hz, 1H), 5.09-5.01 (m, 1H), 4.12 (t, J=8.0 Hz, 2H), 3.77 (s, 3H), 3.69 (s, 2H), 3.46 (s, 4H), 3.01 (s, 3H), 2.75 (dd, J=43.6, 31.7 Hz, 5H), 2.59 (s, 3H), 2.49-2.43 (m, 3H), 2.35 (s, 3H), 2.02 (d, J=14.4 Hz, 7H), 1.86 (d, J=6.7 Hz, 4H), 1.62 (s, 2H), 0.92 (s, 3H); [M+H]⁺=1061.8.

### Example 58: 5-(3-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)azetidin-1-yl)-2-(2,6-dioxopiperidin-3-yl)isoindoline-1,3-dione

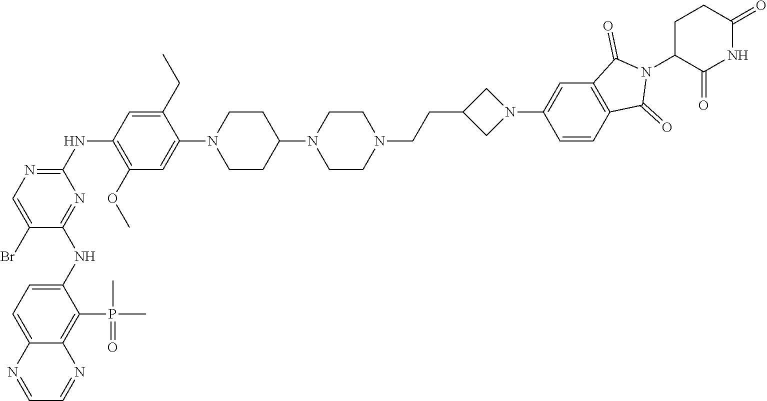 [SMILES: CCc1cc(Nc2ncc(Br)c(Nc3ccc4nccnc4c3P(C)(C)=O)n2)c(OC)cc1N1CCC(N2CCN(CCC3CN(c4ccc5c(c4)C(=O)N(C4CCC(=O)NC4=O)C5=O)C3)CC2)CC1]

The titled compound was synthesized in the procedures similar to Example 56. ¹H NMR (400 MHz, DMSO) δ 12.65 (s, 1H), 11.07 (s, 1H), 8.86 (d, J=4.3 Hz, 3H), 8.27 (s, 2H), 7.92 (s, 1H), 7.64 (d, J=8.3 Hz, 1H), 7.38 (s, 1H), 6.90 (s, 1H), 6.81 (s, 2H), 5.05 (s, 1H), 3.77 (s, 3H), 3.53 (d, J=20.9 Hz, 2H), 3.41 (s, 1H), 3.30-3.27 (m, 1H), 3.14 (s, 1H), 3.01 (s, 3H), 2.89 (s, 2H), 2.70 (d, J=14.9 Hz, 3H), 2.60 (m, 7H), 2.37 (s, 5H), 2.13 (s, 1H), 2.02 (d, J=14.3 Hz, 7H), 1.87 (s, 2H), 1.76 (s, 1H), 1.61 (s, 2H), 0.93 (s, 3H);[M+H]⁺=1033.8.

### Example 59: 5-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)-2-(2,6-dioxopiperidin-3-yl)isoindoline-1,3-dione

### Step 1: 2-(2,6-dioxopiperidin-3-yl)-5-(3-(hydroxymethyl)azetidin-1-yl)isoindoline-1,3-dione

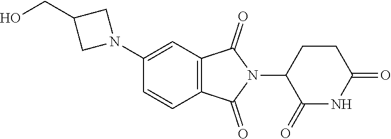 [SMILES: O=C1CCC(N2C(=O)c3ccc(N4CC(CO)C4)cc3C2=O)C(=O)N1]

Into a 100-mL flask, was placed azetidin-3-ylmethanol hydrochloride (500 mg, 4.05 mmol), DMSO (8 mL), 2-(2,6-dioxopiperidin-3-yl)-5-fluoroisoindole-1,3-dione (1.23 g, 4.45 mmol), DIEA (2.61 g, 20.25 mmol). The resulting solution was stirred for 3 hours at 80° C. The reaction mixture was cooled to room temperature. The reaction was quenched with water and extracted with DCM (2×50 mL). The combined organic layers were washed with brine, dried over anhydrous Na₂SO₄, and evaporated in vacuum to afford the crude product, which was further purified with silica gel column chromatography (DCM: MeOH=100: 0˜ 90: 10 gradient elution) to give the product (600 mg, 43%). [M+H]⁺=344.1.

### Step 2: (1-(2-(2,6-dioxopiperidin-3-yl)-1,3-dioxoisoindolin-5-yl)azetidin-3-yl)methyl methanesulfonate

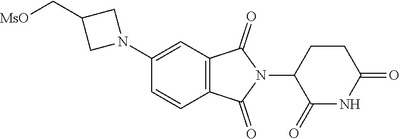 [SMILES: CS(=O)(=O)OCC1CN(c2ccc3c(c2)C(=O)N(C2CCC(=O)NC2=O)C3=O)C1]

To a solution of 2-(2,6-dioxopiperidin-3-yl)-5-(3-(hydroxymethyl)azetidin-1-yl)isoindoline-1,3-dione (300 mg, 0.875 mmol) in DCM (10 mL) was added TEA (353 mg, 3.498 mmol). The reaction mixture was cool down to 0° C., then MsCl (152 mg, 1.313 mmol) was added. The mixture was stirred at 25° C. for 3 hrs. The reaction was quenched with water and extracted with DCM (2×30 mL). The combined organic layers were washed with brine, dried over anhydrous Na₂SO₄, and evaporated in vacuum to afford the crude product, which was further purified with silica gel column chromatography (DCM: MeOH=100: 0˜20: 1 gradient elution) to give the product (180 mg, 49%). [M+H]⁺=422.3.

### Step 3: 5-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)-2-(2,6-dioxopiperidin-3-yl)isoindoline-1,3-dione

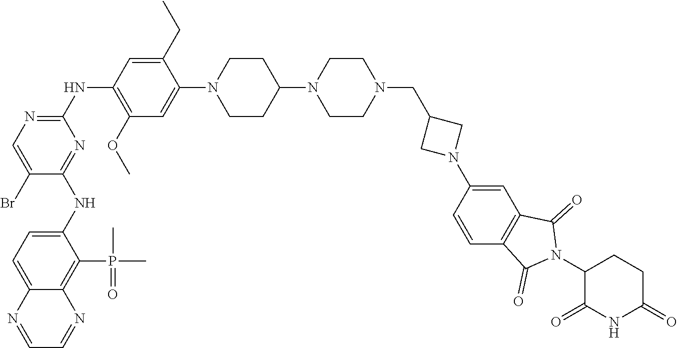 [SMILES: CCc1cc(Nc2ncc(Br)c(Nc3ccc4nccnc4c3P(C)(C)=O)n2)c(OC)cc1N1CCC(N2CCN(CC3CN(c4ccc5c(c4)C(=O)N(C4CCC(=O)NC4=O)C5=O)C3)CC2)CC1]

The titled compound was synthesized in the procedures similar to Example 56 step 4.

¹H NMR (400 MHz, DMSO) δ 12.65 (s, 1H), 11.07 (s, 1H), 8.87 (d, J=4.3 Hz, 3H), 8.27 (s, 2H), 7.92 (s, 1H), 7.63 (d, J=8.4 Hz, 1H), 7.38 (s, 1H), 6.80 (d, J=13.3 Hz, 2H), 6.65 (d, J=8.3 Hz, 1H), 5.06 (d, J=12.3 Hz, 1H), 4.13 (s, 2H), 3.77 (s, 3H), 3.69 (s, 2H), 3.00 (s, 4H), 2.88 (s, 2H), 2.69 (d, J=13.4 Hz, 3H), 2.57 (m, 7H), 2.43 (s, 3H), 2.33 (s, 2H), 2.02 (d, J=14.3 Hz, 7H), 1.85 (s, 2H), 1.58 (d, J=10.5 Hz, 2H), 0.93 (s, 3H);[M+H]⁺=1019.8.

### Example 60: 5-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propyl)piperazin-1-yl)-2-(2,6-dioxopiperidin-3-yl)isoindoline-1,3-dione

### Step 1: 2-(2,6-dioxopiperidin-3-yl)-5-(4-(3-hydroxypropyl)piperazin-1-yl)isoindoline-1,3-dione

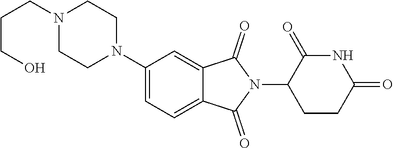 [SMILES: O=C1CCC(N2C(=O)c3ccc(N4CCN(CCCO)CC4)cc3C2=O)C(=O)N1]

Into a 50-mL flask, was placed 3-(piperazin-1-yl)propan-1-ol (1.0 g, 6.9 mmol), DMSO (10 mL), 2-(2,6-dioxopiperidin-3-yl)-5-fluoroisoindole-1,3-dione (2.09 g, 7.59 mmol), DIEA (2.67 g, 20.7 mmol). The resulting solution was stirred for 1 h at 80° C. The reaction mixture was cooled to room temperature. The resulting solution was diluted with EA. The resulting solution was extracted with H₂O and the organic layers were combined and dried over anhydrous sodium sulfate and concentrated under vacuum. The residue was applied onto a silica gel column with dichloromethane/methanol (8:1) to afford the product (620 mg, 20%). [M+H]⁺=401.27.

### Step 2: 3-(4-(2-(2,6-dioxopiperidin-3-yl)-1,3-dioxoisoindolin-5-yl)piperazin-1-yl)propyl 4-methylbenzenesulfonate

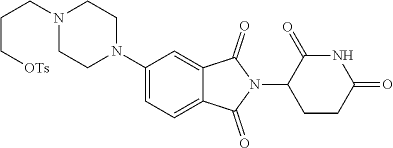 [SMILES: Cc1ccc(S(=O)(=O)OCCCN2CCN(c3ccc4c(c3)C(=O)N(C3CCC(=O)NC3=O)C4=O)CC2)cc1]

Into a 25-mL flask, was placed 2-(2,6-dioxopiperidin-3-yl)-5-(4-(3-hydroxypropyl)piperazin-1-yl)isoindoline-1,3-dione (620 mg, 1.55 mmol), DCM (12 mL), TEA (469.6 mg, 4.65 mmol), TsCl (354 mg, 1.86 mmol). The resulting solution was stirred at room temperature for 16 h. The resulting mixture was concentrated under vacuum. The residue was applied onto a silica gel column with dichloromethane/methanol (7:1) to afford the product (410 mg, 47.7%). [M+H]⁺=555.28.

### Step 3: 5-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propyl)piperazin-1-yl)-2-(2,6-dioxopiperidin-3-yl)isoindoline-1,3-dione

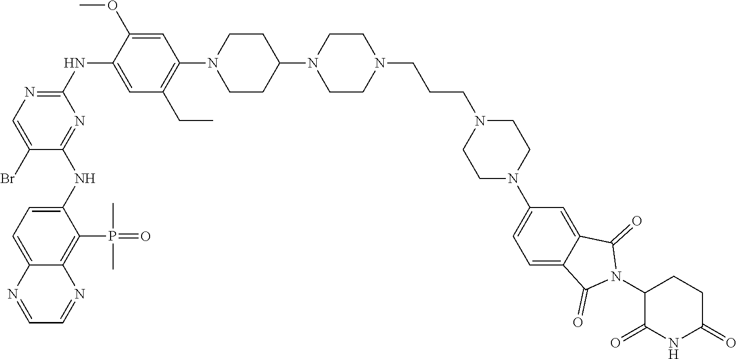 [SMILES: CCc1cc(Nc2ncc(Br)c(Nc3ccc4nccnc4c3P(C)(C)=O)n2)c(OC)cc1N1CCC(N2CCN(CCCN3CCN(c4ccc5c(c4)C(=O)N(C4CCC(=O)NC4=O)C5=O)CC3)CC2)CC1]

The titled compound was prepared in a manner similar to that in Example 56 step 4. ¹H NMR (400 MHz, DMSO) δ 12.65 (s, 1H), 11.09 (s, 1H), 8.87 (d, J=4.6 Hz, 3H), 8.40-8.21 (m, 3H), 7.91 (s, 1H), 7.68 (d, J=8.0 Hz, 1H), 7.36 (d, J=10.2 Hz, 2H), 7.27 (s, 1H), 6.81 (s, 1H), 5.06 (s, 1H), 3.77 (s, 3H), 3.44 (s, 4H), 2.95-3.00 (m, 7H), 2.79-2.56 (m, 5H), 2.33-2.35 (s, 12H), 2.02 (d, J=14.6 Hz, 7H), 1.85-1.87 (m, 3H), 1.62-1.66 (m, 4H), 0.93 (s, 3H); [M+H]+=1076.

### Example 61: 5-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-3-oxopropyl)piperazin-1-yl)-2-(2,6-dioxopiperidin-3-yl)isoindoline-1,3-dione

### Step 1: tert-butyl 3-(4-(2-(2,6-dioxopiperidin-3-yl)-1,3-dioxoisoindolin-5-yl)piperazin-1-yl)propanoate

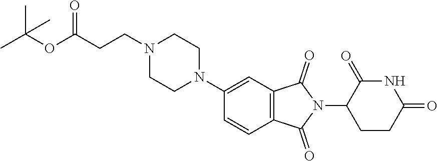 [SMILES: CC(C)(C)OC(=O)CCN1CCN(c2ccc3c(c2)C(=O)N(C2CCC(=O)NC2=O)C3=O)CC1]

A mixture of 2-(2,6-dioxopiperidin-3-yl)-5-(piperazin-1-yl)isoindoline-1,3-dione (300 mg, 0.88 mmol) (The intermediate can be prepared according to the method described in US 20180125821), tert-butyl acrylate (337 mg, 2.63 mmol) and DIEA (226 mg, 1.76 mmol) in MeCN (8 mL) was stirred in a flask at 80° C. overnight. The mixture was evaporated in vacuum to afford the crude product, which was further purified with silica gel column chromatography (DCM: MeOH=100: 0˜ 10: 1 gradient elution) to give the product (280 mg, 68%). [M+H]⁺=471.2.

### Step 2: 3-(4-(2-(2,6-dioxopiperidin-3-yl)-1,3-dioxoisoindolin-5-yl)piperazin-1-yl) propanoic acid

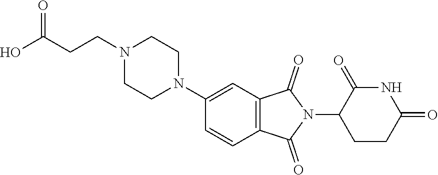 [SMILES: O=C(O)CCN1CCN(c2ccc3c(c2)C(=O)N(C2CCC(=O)NC2=O)C3=O)CC1]

A solution of tert-butyl 3-(4-(2-(2,6-dioxopiperidin-3-yl)-1,3-dioxoisoindolin-5-yl)piperazin-1-yl)propanoate (280 mg, 0.59 mmol) in HCl/1,4-dioxane (4M, 8 mL) was stirred in a flask at room temperature overnight. The mixture was evaporated in vacuum to afford the crude product (245 mg, 99%), which was used for next step without further purification. [M+H]⁺=415.2.

### Step 3: 5-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-3-oxopropyl)piperazin-1-yl)-2-(2,6-dioxopiperidin-3-yl)isoindoline-1,3-dione

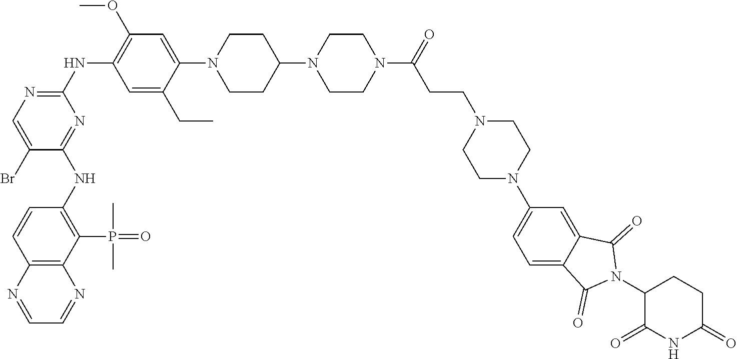 [SMILES: CCc1cc(Nc2ncc(Br)c(Nc3ccc4nccnc4c3P(C)(C)=O)n2)c(OC)cc1N1CCC(N2CCN(C(=O)CCN3CCN(c4ccc5c(c4)C(=O)N(C4CCC(=O)NC4=O)C5=O)CC3)CC2)CC1]

The titled compound was prepared in a manner similar to that in Example 24 step 6.

¹H NMR (400 MHz, DMSO) δ 12.65 (s, 1H), 11.09 (s, 1H), 8.86 (d, J=6.4 Hz, 3H), 8.28 (d, J=8.4 Hz, 2H), 8.20 (s, 1H), 7.91 (s, 1H), 7.68 (d, J=8.8 Hz, 1H), 7.35 (s, 2H), 7.27 (d, J=8.5 Hz, 1H), 6.81 (s, 1H), 5.07 (d, J=7.8 Hz, 1H), 3.77 (s, 3H), 3.44 (m, 8H), 2.94 (m, 7H), 2.44-2.54 (m, 12H), 2.42-2.36 (m, 1H), 2.02 (d, J=14.2 Hz, 7H), 1.80-1.83 (m, 2H), 1.60-1.62 (m, 2H), 1.23 (s, 1H), 0.92 (t, 3H); [M+H]⁺=1090.4.

### Example 62: 5-(4-(5-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-5-oxopentyl)piperazin-1-yl)-2-(2,6-dioxopiperidin-3-yl)isoindoline-1,3-dione

### Step 1: benzyl 4-(5-(tert-butoxy)-5-oxopentyl)piperazine-1-carboxylate

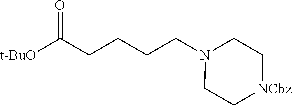 [SMILES: CC(C)(C)OC(=O)CCCCN1CCN(C(=O)OCc2ccccc2)CC1]

A solution of tert-butyl 5-bromopentanoate (2.00 g, 8.434 mmol), benzyl piperazine-1-carboxylate (1.86 g, 8.434 mmol) and DIEA (2.18 g, 16.868 mmol) in acetonitrile (20 mL) was stirred for 1 h at 85° C. The resulting mixture was extracted with EtOAc. The combined organic layers were washed with brine, dried over anhydrous Na₂SO₄. After filtration, the filtrate was concentrated under reduced pressure to afford the crude product (2.52 g) which was used in the next step directly without further purification. [M+H]⁺=377.2.

### Step 2: tert-butyl 5-(piperazin-1-yl)pentanoate

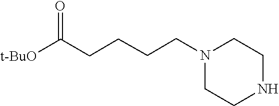 [SMILES: CC(C)(C)OC(=O)CCCCN1CCNCC1]

A solution of benzyl 4-(5-(tert-butoxy)-5-oxopentyl)piperazine-1-carboxylate (2.50 g, crude), AcOH (20 mL) and Pd/C (1.20 g, 10% wt) in MeOH (20 mL) was stirred for 1 h at room temperature under hydrogen atmosphere. The resulting mixture was filtered, the filter cake was washed with MeOH. The filtrate was concentrated under reduced pressure to afford the crude product (1.53 g) which was used in the next step directly without further purification. [M+H]⁺=243.2.

### Step 3: tert-butyl 5-(4-(2-(2,6-dioxopiperidin-3-yl)-1,3-dioxoisoindolin-5-yl)piperazin-1-yl)pentanoate

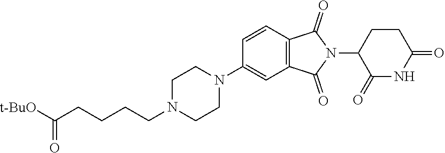 [SMILES: CC(C)(C)OC(=O)CCCCN1CCN(c2ccc3c(c2)C(=O)N(C2CCC(=O)NC2=O)C3=O)CC1]

A solution of tert-butyl 5-(piperazin-1-yl)pentanoate (600 mg, crude), 2-(2,6-dioxopiperidin-3-yl)-5-fluoroisoindoline-1,3-dione (684 mg, 2.48 mmol) and DIEA (1.60 g, 12.4 mmol) in DMSO (6 mL) was stirred for 1 h at 80° C. The resulting mixture was extracted with EtOAc. The combined organic layers were washed with brine, dried over anhydrous Na₂SO₄. After filtration, the filtrate was concentrated under reduced pressure to afford the crude product (1.12 g) which was used in the next step directly without further purification [M+H]⁺=499.3.

### Step 4: 5-(4-(2-(2,6-dioxopiperidin-3-yl)-1,3-dioxoisoindolin-5-yl)piperazin-1-yl)pentanoic acid

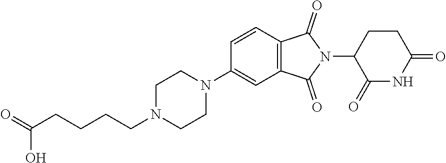 [SMILES: O=C(O)CCCCN1CCN(c2ccc3c(c2)C(=O)N(C2CCC(=O)NC2=O)C3=O)CC1]

A solution of tert-butyl 5-(4-(2-(2,6-dioxopiperidin-3-yl)-1,3-dioxoisoindolin-5-yl)piperazin-1-yl)pentanoate (800 mg, crude) and TFA (8 mL) in DCM (8 mL) was stirred for 1 h at room temperature. The resulting mixture was concentrated under vacuum. The resulting mixture was diluted with water. The resulting mixture was concentrated under vacuum to afford the crude product (715 mg) which was used in the next step directly without further purification. [M+H]⁺=443.2.

### Step 5. 5-(4-(5-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-5-oxopentyl)piperazin-1-yl)-2-(2,6-dioxopiperidin-3-yl)isoindoline-1,3-dione

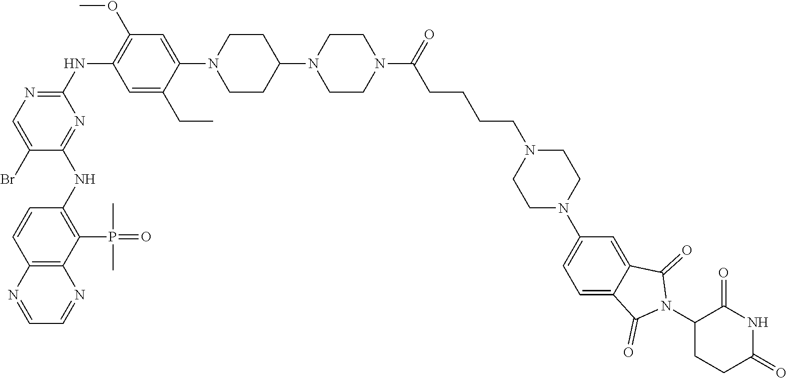 [SMILES: CCc1cc(Nc2ncc(Br)c(Nc3ccc4nccnc4c3P(C)(C)=O)n2)c(OC)cc1N1CCC(N2CCN(C(=O)CCCCN3CCN(c4ccc5c(c4)C(=O)N(C4CCC(=O)NC4=O)C5=O)CC3)CC2)CC1]

The titled compound was prepared in a manner similar to that in Example 24 step 6.

¹H NMR (400 MHz, CD3OD) δ 8.92 (s, 2H), 8.24-8.26 (m, 2H), 7.77 (d, J=8.1 Hz, 1H), 7.49 (s, 1H), 7.37 (d, J=7.4 Hz, 1H), 7.30 (s, 1H), 6.91 (s, 1H), 5.09 (s, 1H), 4.79 (s, 2H), 4.29 (s, 1H), 4.21 (d, J=12.6 Hz, 2H), 3.85 (s, 3H), 3.74 (d, J=10.2 Hz, 4H), 3.65-3.56 (m, 1H), 3.48-3.50 (m, 2H), 3.15 (d, J=13.7 Hz, 3H), 2.90 (d, J=13.3 Hz, 3H), 2.74 (d, J=13.9 Hz, 3H), 2.65-2.67 (m, 9H), 2.34 (s, 3H), 2.16 (d, J=14.4 Hz, 7H), 2.03 (s, 2H), 1.89 (s, 2H), 1.76 (s, 2H), 1.04-1.06 (m, 3H); [M+H]⁺=1118.

### Example 63: 5-(4-((4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)piperidin-1-yl)methyl)piperidin-1-yl)-2-(2,6-dioxopiperidin-3-yl)isoindoline-1,3-dione

### Step 1: benzyl 4-((4-(tert-butoxycarbonyl)piperidin-1-yl)methyl)piperidine-1-carboxylate

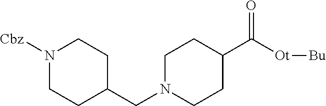

To a stirred solution of benzyl 4-formylpiperidine-1-carboxylate (1.01 g, 4.044 mmol), tert-butyl piperidine-4-carboxylate hydrochloride (897 mg, 4.044 mmol), AcONa (3.31 g, 40.4 mmol,) and DCM (20 mL) was added STAB (5.14 g, 24.263 mmol) in portions at 0° C. The resulting mixture was stirred overnight at room temperature. The reaction was quenched with water at room temperature. The resulting mixture was extracted with CH₂Cl₂. The combined organic layers were washed with brine, dried over anhydrous Na₂SO₄.

After filtration, the filtrate was concentrated under reduced pressure to afford the crude product (1.20 g) which was used in the next step directly without further purification. [M+H]⁺=417.3.

### Step 2: tert-butyl 1-(piperidin-4-ylmethyl)piperidine-4-carboxylate

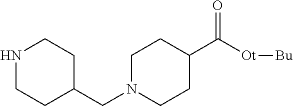

A suspension of benzyl 4-((4-(tert-butoxycarbonyl)piperidin-1-yl)methyl)piperidine-1-carboxylate (1.20 g, crude), AcOH (20 mL) and Pd/C (1.20 g, 10% wt) in MeOH (20 mL) was stirred for 1 h at room temperature under hydrogen atmosphere. The resulting mixture was filtered, the filter cake was washed with MeOH. The filtrate was concentrated under reduced pressure to afford the crude product (900 mg) which was used in the next step directly without further purification. [M+H]⁺=283.2.

### Step 3: tert-butyl 1-((1-(2-(2,6-dioxopiperidin-3-yl)-1,3-dioxoisoindolin-5-yl)piperidin-4-yl)methyl)piperidine-4-carboxylate

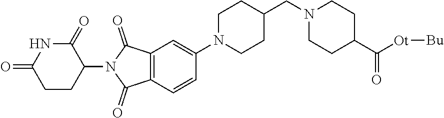

A solution of tert-butyl 1-(piperidin-4-ylmethyl)piperidine-4-carboxylate (500 mg, crude) and 2-(2,6-dioxopiperidin-3-yl)-5-fluoroisoindoline-1,3-dione (489 mg, 1.770 mmol), DIEA (1.14 g, 8.852 mmol) in DMSO (6 mL) was stirred for 1 h at 80° C. The resulting mixture was extracted with EtOAc. The combined organic layers were washed with brine, dried over anhydrous Na₂SO₄. After filtration, the filtrate was concentrated under reduced pressure to afford the crude product (830 mg) which was used in the next step directly without further purification. [M+H]⁺=539.3.

### Step 4: 1-((1-(2-(2,6-dioxopiperidin-3-yl)-1,3-dioxoisoindolin-5-yl)piperidin-4-yl)methyl)piperidine-4-carboxylic acid

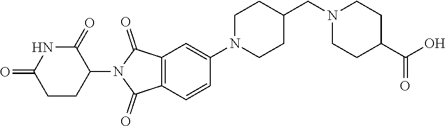 [SMILES: O=C1CCC(N2C(=O)c3ccc(N4CCC(CN5CCC(C(=O)O)CC5)CC4)cc3C2=O)C(=O)N1]

A solution of tert-butyl 1-((1-(2-(2,6-dioxopiperidin-3-yl)-1,3-dioxoisoindolin-5-yl) piperidin-4-yl)methyl)piperidine-4-carboxylate (810.00 mg, crude) and TFA (8 mL) in DCM (8 mL) was stirred for 1 h at room temperature. The resulting mixture was concentrated under vacuum. The resulting mixture was diluted with water. The resulting mixture was concentrated under vacuum to afford the crude product (725 mg) which was used in the next step directly without further purification. [M+H]⁺=483.2.

### Step 5. 5-(4-((4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)piperidin-1-yl)methyl)piperidin-1-yl)-2-(2,6-dioxopiperidin-3-yl)isoindoline-1,3-dione

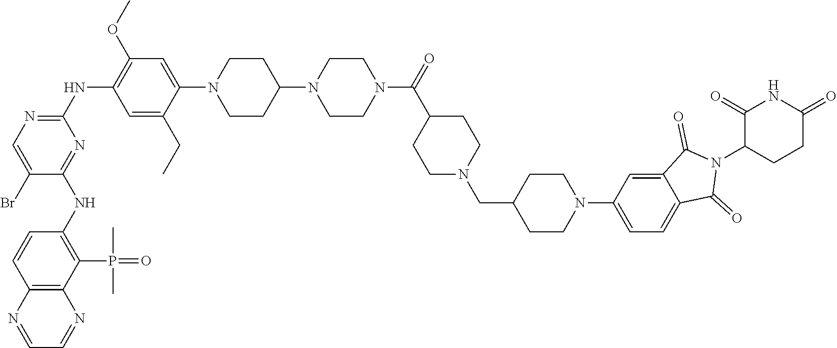 [SMILES: CCc1cc(Nc2ncc(Br)c(Nc3ccc4nccnc4c3P(C)(C)=O)n2)c(OC)cc1N1CCC(N2CCN(C(=O)C3CCN(CC4CCN(c5ccc6c(c5)C(=O)N(C5CCC(=O)NC5=O)C6=O)CC4)CC3)CC2)CC1]

The titled compound was prepared in a manner similar to that in Example 24 step 6.

¹H NMR (400 MHz, DMSO) δ 12.65 (s, 1H), 11.09 (s, 1H), 8.86 (s, 2H), 8.36-8.11 (m, 3H), 7.88 (s, 1H), 7.64 (s, 1H), 7.37 (s, 1H), 7.31 (s, 1H), 7.24 (s, 1H), 6.81 (s, 1H), 5.06 (s, 1H), 4.03 (s, 2H), 3.77 (s, 3H), 3.47 (s, 4H), 3.11-2.77 (m, 9H), 2.77-2.58 (m, 4H), 2.33 (m, 6H), 2.12 (s, 2H), 2.02 (d, J=14.3 Hz, 6H), 1.95-1.71 (m, 7H), 1.58 (s, 6H), 1.14 (m, 2H), 0.92 (s, 3H); [M+H]⁺=1158.

### Example 68: 3-(6-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-2-oxoethyl)piperazin-1-yl)-1-oxoisoindolin-2-yl)piperidine-2,6-dione

### Step 1: tert-butyl 2-(4-(2-(2,6-dioxopiperidin-3-yl)-1-oxoisoindolin-5-yl)piperazin-1-yl)acetate

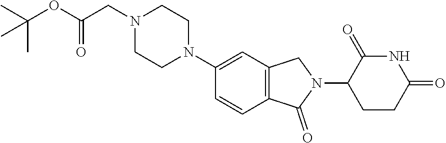 [SMILES: CC(C)(C)OC(=O)CN1CCN(c2ccc3c(c2)CN(C2CCC(=O)NC2=O)C3=O)CC1]

A mixture of 3-(1-oxo-5-(piperazin-1-yl)isoindolin-2-yl)piperidine-2,6-dione (The synthesis of this intermediate is described in WO2020051564) (300 mg, 0.91 mmol), tert-butyl 2-bromoacetate (890 mg, 4.6 mmol) and TEA (185 mg, 1.82 mmol) in THF (10 mL) was stirred in a flask at 60° C. for 3 hours. The mixture was evaporated in vacuum to afford the crude product, which was further purified with silica gel column chromatography (DCM: MeOH=100: 0˜ 10: 1 gradient elution) to give the title product (280 mg, 69%). [M+H]⁺=443.2.

### Step 2: 2-(4-(2-(2,6-dioxopiperidin-3-yl)-1-oxoisoindolin-5-yl)piperazin-1-yl)acetic acid

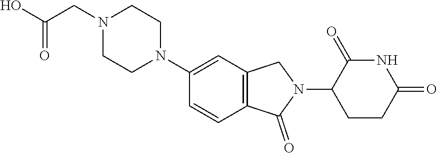 [SMILES: O=C(O)CN1CCN(c2ccc3c(c2)CN(C2CCC(=O)NC2=O)C3=O)CC1]

A solution of tert-butyl 2-(4-(2-(2,6-dioxopiperidin-3-yl)-1-oxoisoindolin-5-yl)piperazin-1-yl)acetate (280 mg, 0.63 mmol) in HCl/1,4-dioxane (10 mL) was stirred in a flask at room temperature overnight. The mixture was evaporated in vacuum to afford the crude product (240 mg, 98%), which was used for next step without further purification. [M+H]⁺=387.2.

### Step 3: 3-(6-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-2-oxoethyl)piperazin-1-yl)-1-oxoisoindolin-2-yl)piperidine-2,6-dione

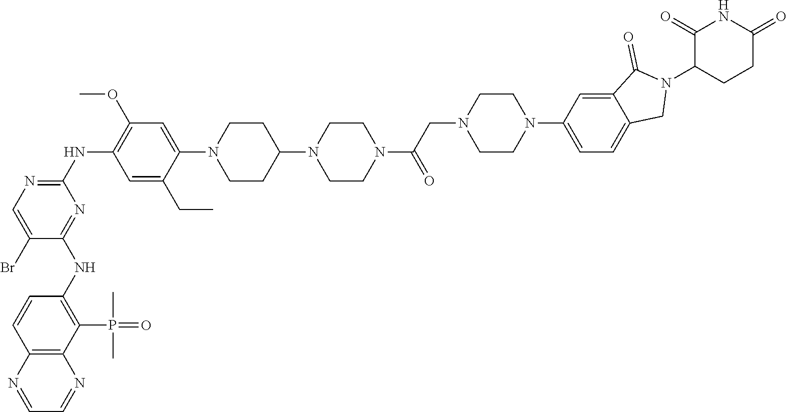 [SMILES: CCc1cc(Nc2ncc(Br)c(Nc3ccc4nccnc4c3P(C)(C)=O)n2)c(OC)cc1N1CCC(N2CCN(C(=O)CN3CCN(c4ccc5c(c4)C(=O)N(C4CCC(=O)NC4=O)C5)CC3)CC2)CC1]

To a solution of 2-(4-(2-(2,6-dioxopiperidin-3-yl)-1-oxoisoindolin-5-yl)piperazin-1-yl)acetic acid (23 mg, 0.06 mmol), HATU (23 mg, 0.06 mmol) and DIEA (20 mg, 0.15 mmol) in DMF (2 mL) was added (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (35 mg, 0.05 mmol). The resulting mixture was stirred at room temperature for 1 h. The reaction was directly purified with pre-HPLC to give the product (9 mg, 14%). ¹H NMR (400 MHz, DMSO) δ 12.65 (s, 1H), 10.71 (s, 1H), 8.85 (d, J=12.0 Hz, 3H), 8.28 (d, J=11.7 Hz, 2H), 7.90 (s, 1H), 7.53 (d, J=8.6 Hz, 1H), 7.36 (s, 1H), 7.07 (d, J=9.4 Hz, 1H), 7.01 (s, 1H), 6.80 (s, 1H), 5.30 (s, 2H), 4.58-4.53 (m, 1H), 3.76 (s, 3H), 3.58 (s, 2H), 3.47 (s, 2H), 3.22 (s, 3H), 3.00 (s, 4H), 2.40-2.57 (m, 12H), 2.36 (d, J=21.9 Hz, 3H), 2.02 (d, J=14.3 Hz, 8H), 1.84 (s, 2H), 1.61 (s, 2H), 1.23 (s, 1H), 0.92 (s, 3H); [M+H]⁺=1062.

### Example 74: 3-(6-(2-((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)ethyl)pyridin-3-yl)piperidine-2,6-dione

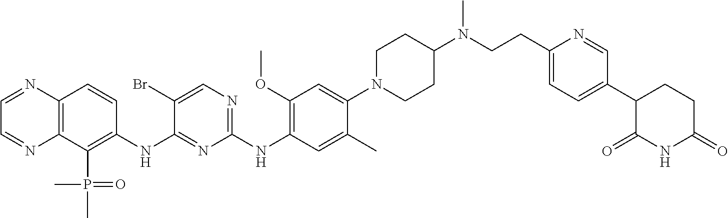 [SMILES: COc1cc(N2CCC(N(C)CCc3ccc(C4CCC(=O)NC4=O)cn3)CC2)c(C)cc1Nc1ncc(Br)c(Nc2ccc3nccnc3c2P(C)(C)=O)n1]

The titled compound was synthesized in the procedures similar to Example 13. ¹H NMR (400 MHz, DMSO) δ 12.69 (s, 1H), 10.90 (s, 1H), 8.86 (s, 3H), 8.36 (s, 1H), 8.27 (s, 2H), 7.94 (s, 1H), 7.56 (s, 1H), 7.35 (s, 2H), 6.75 (s, 1H), 3.91 (d, J=12.0 Hz, 1H), 3.77 (s, 3H), 3.07 (s, 3H), 2.85-2.88 (m, 4H), 2.66 (s, 3H), 2.53 (s, 2H), 2.32 (s, 3H), 2.18-2.26 (m, 1H), 2.09 (s, 3H), 2.03 (d, J=13.8 Hz, 6H), 1.79 (m, 2H), 1.61 (m, 2H); [M+H]⁺=841.

### Example 75: 3-(4-(2-((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)ethyl)phenyl)piperidine-2,6-dione

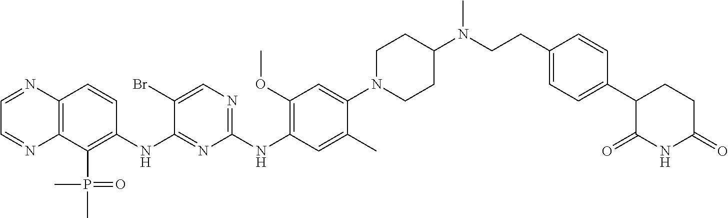 [SMILES: COc1cc(N2CCC(N(C)CCc3ccc(C4CCC(=O)NC4=O)cc3)CC2)c(C)cc1Nc1ncc(Br)c(Nc2ccc3nccnc3c2P(C)(C)=O)n1]

The titled compound was synthesized in the procedures similar to Example 23. ¹H NMR (400 MHz, DMSO) δ 12.69 (s, 1H), 10.83 (s, 1H), 8.86 (d, J=9.0 Hz, 3H), 8.27 (s, 2H), 7.91 (s, 1H), 7.35 (s, 1H), 7.21 (s, 2H), 7.15 (s, 2H), 6.75 (s, 1H), 3.77 (s, 4H), 3.08 (s, 2H), 2.71 (d, J=28.8 Hz, 8H), 2.36 (s, 4H), 2.19 (s, 1H), 2.09 (s, 3H), 2.03 (d, J=14.2 Hz, 7H), 1.80 (s, 2H), 1.65 (s, 2H), 1.24 (s, 2H); [M+H]⁺=840.

### Example 76: 3-(4-(2-((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)ethoxy)phenyl)piperidine-2,6-dione

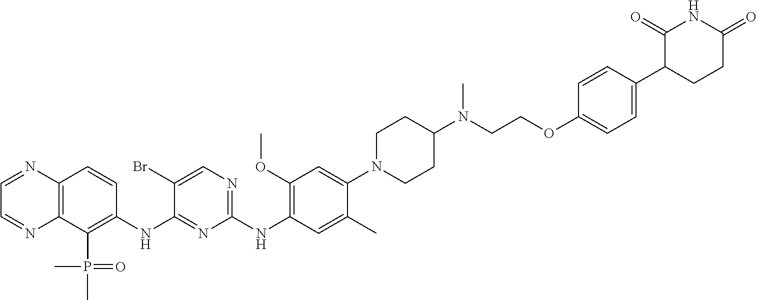 [SMILES: COc1cc(N2CCC(N(C)CCOc3ccc(C4CCC(=O)NC4=O)cc3)CC2)c(C)cc1Nc1ncc(Br)c(Nc2ccc3nccnc3c2P(C)(C)=O)n1]

The titled compound was synthesized in the procedures similar to Example 18. ¹H NMR (400 MHz, DMSO) δ 12.69 (s, 1H), 10.81 (s, 1H), 8.86 (d, J=8.6 Hz, 3H), 8.27 (s, 2H), 7.93 (d, J=7.8 Hz, 1H), 7.35 (s, 1H), 7.13 (d, J=8.3 Hz, 2H), 6.92 (d, J=8.5 Hz, 2H), 6.76 (s, 1H), 4.05 (s, 2H), 3.77 (s, 4H), 3.11 (d, J=9.7 Hz, 2H), 2.86 (s, 2H), 2.66 (d, J=10.3 Hz, 3H), 2.52 (s, 1H), 2.48-2.42 (m, 1H), 2.34 (s, 3H), 2.16 (d, J=9.6 Hz, 1H), 2.10 (s, 3H), 2.02 (d, J=14.3 Hz, 7H), 1.83 (d, J=12.4 Hz, 2H), 1.64 (d, J=10.9 Hz, 2H); [M+H]⁺=856.

### Example 77: 3-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)phenyl)piperidine-2,6-dione

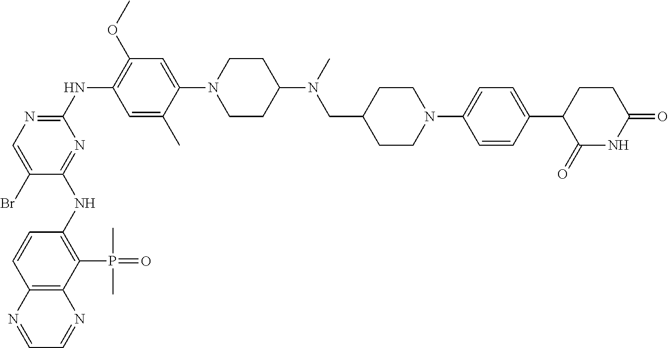 [SMILES: COc1cc(N2CCC(N(C)CC3CCN(c4ccc(C5CCC(=O)NC5=O)cc4)CC3)CC2)c(C)cc1Nc1ncc(Br)c(Nc2ccc3nccnc3c2P(C)(C)=O)n1]

The titled compound was synthesized in the procedures similar to Example 23. ¹H NMR (400 MHz, DMSO) δ 12.70 (s, 1H), 10.79 (s, 1H), 8.87 (d, J=10.2 Hz, 3H), 8.28 (s, 2H), 7.94 (s, 1H), 7.37 (s, 1H), 7.03 (s, 2H), 6.92 (s, 2H), 6.76 (s, 1H), 3.78 (s, 6H), 3.13-3.28 (m, 6H), 2.51-2.70 (m, 6H), 2.11 (s, 4H), 2.03-2.05 (m, 8H), 1.62-1.88 (m, 6H), 1.18-1.26 (m, 3H); [M+H]⁺=909.

### Example 78: 3-(4-(4-(2-((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)ethyl)piperidin-1-yl)phenyl)piperidine-2,6-dione

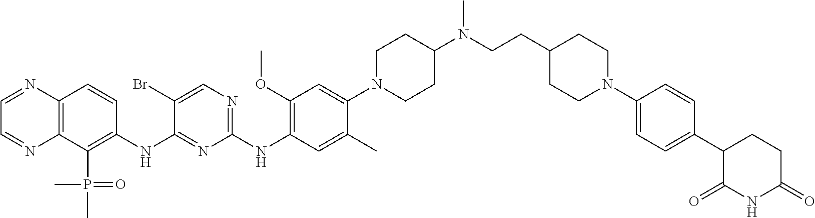 [SMILES: COc1cc(N2CCC(N(C)CCC3CCN(c4ccc(C5CCC(=O)NC5=O)cc4)CC3)CC2)c(C)cc1Nc1ncc(Br)c(Nc2ccc3nccnc3c2P(C)(C)=O)n1]

The titled compound was synthesized in the procedures similar to Example 23. ¹H NMR (400 MHz, DMSO) δ 12.69 (s, 1H), 10.78 (s, 1H), 8.87 (s, 3H), 8.27 (s, 2H), 7.94 (s, 1H), 7.03 (d, J=7.4 Hz, 2H), 6.90 (s, 2H), 6.76 (s, 1H), 3.77 (m, 8H), 3.09 (m, 2H), 2.67 (m, 6H), 2.24 (s, 3H), 2.10 (s, 3H), 2.03 (d, J=14.2 Hz, 6H), 1.79 (m, 3H), 1.64 (m, 2H), 1.41 (m, 3H), 1.24 (s, 6H); [M+H]⁺=923.

### Example 80: 1-(4-(4-(2-((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)ethyl)piperidin-1-yl)phenyl)dihydropyrimidine-2,4(1H,3H)-dione

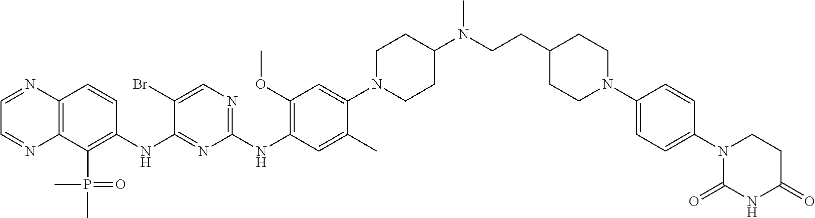 [SMILES: COc1cc(N2CCC(N(C)CCC3CCN(c4ccc(N5CCC(=O)NC5=O)cc4)CC3)CC2)c(C)cc1Nc1ncc(Br)c(Nc2ccc3nccnc3c2P(C)(C)=O)n1]

The titled compound was synthesized in the procedures similar to Example 79. ¹H NMR (400 MHz, DMSO) δ 12.69 (s, 1H), 10.26 (s, 1H), 8.87 (d, J=7.8 Hz, 3H), 8.27 (s, 2H), 7.94 (s, 1H), 7.35 (s, 1H), 7.13 (d, J=8.0 Hz, 2H), 6.93 (d, J=7.3 Hz, 2H), 6.76 (s, 1H), 3.77 (s, 3H), 3.69 (s, 4H), 3.09 (s, 3H), 2.66 (d, J=12.3 Hz, 6H), 2.52 (s, 2H), 2.24 (s, 3H), 2.10 (s, 3H), 2.03 (d, J=14.2 Hz, 6H), 1.76-1.79 (m, 4H), 1.55-1.64 (m, 2H), 1.38-1.50 (m, 3H), 1.19-1.30 (m, 2H); [M+H]⁺=924.

### Example 81: 1-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)-2-fluorophenyl)dihydropyrimidine-2,4(1H,3H)-dione

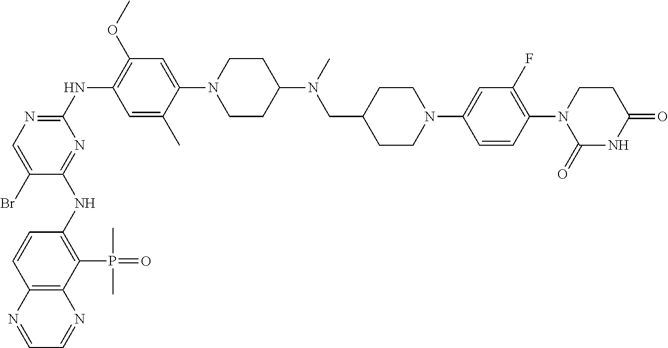 [SMILES: COc1cc(N2CCC(N(C)CC3CCN(c4ccc(N5CCC(=O)NC5=O)c(F)c4)CC3)CC2)c(C)cc1Nc1ncc(Br)c(Nc2ccc3nccnc3c2P(C)(C)=O)n1]

The titled compound was synthesized in the procedures similar to Example 79. ¹H NMR (400 MHz, DMSO) δ 12.67 (s, 1H), 10.37 (s, 1H), 8.84 (dd, J=8.4, 1.9 Hz, 3H), 8.25 (d, J=4.8 Hz, 2H), 7.90 (d, J=8.6 Hz, 1H), 7.32 (s, 1H), 7.16 (s, 1H), 6.74 (s, 3H), 3.75 (s, 5H), 3.60 (t, J=6.7 Hz, 2H), 3.07 (d, J=11.2 Hz, 2H), 2.67 (t, J=6.7 Hz, 6H), 2.27 (d, J=24.6 Hz, 6H), 2.07 (s, 3H), 2.00 (d, J=14.4 Hz, 6H), 1.76 (s, 4H), 1.61 (s, 3H), 1.16 (d, J=11.3 Hz, 2H); [M+H]⁺=928.

### Example 82: 1-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)-3-fluorophenyl)dihydropyrimidine-2,4(1H,3H)-dione

### Step 1: (1-(2-fluoro-4-nitrophenyl)piperidin-4-yl)methanol

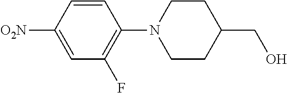 [SMILES: O=[N+]([O-])c1ccc(N2CCC(CO)CC2)c(F)c1]

A mixture of 1,2-difluoro-4-nitrobenzene (90.0 g, 566.0 mmol), piperidin-4-ylmethanol (90.0 g, 782.6 mmol), and TEA (90.0 g, 891.1 mmol) in MeOH (1 L) was stirred at 60° C. for 12 hours. The reaction mixture was cooled down to rt and concentrated. The residue was treated with water (500 mL) and filtered. The filtered cake was slurried with EA (50 mL) and dried to afford example 82-compound 2 (130 g, yield: 90%). [M+H]⁺=255.1.

### Step 2: (1-(4-amino-2-fluorophenyl)piperidin-4-yl)methanol

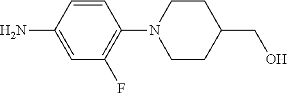 [SMILES: Nc1ccc(N2CCC(CO)CC2)c(F)c1]

To a solution of (1-(2-fluoro-4-nitrophenyl)piperidin-4-yl)methanol (130 g, 511.8 mmol) in EtOH (1.5 L) was added Pd/C (15 g) at rt. The reaction mixture was stirred at 50° C. for 16 hours under H₂ (1 atm). The reaction mixture was cooled to rt, filtered and concentrated to afford the product (90 g, yield: 78%). [M+H]⁺=225.1.

### Step 3: 3-((3-fluoro-4-(4-(hydroxymethyl)piperidin-1-yl)phenyl)amino) propanoic acid

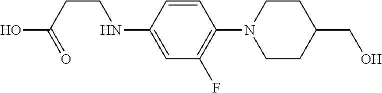 [SMILES: O=C(O)CCNc1ccc(N2CCC(CO)CC2)c(F)c1]

A mixture of (1-(4-amino-2-fluorophenyl)piperidin-4-yl)methanol (40.0 g, 178 mmol) and acrylic acid (40.0 g, 555 mmol) in toluene (500 mL) was stirred at 100° C. for 3 hours. The reaction mixture was cooled down to rt and filtered. The filtered cake was dried to afford the product (55 g, crude). [M+H]⁺=297.2.

### Step 4: (1-(4-(2,4-dioxotetrahydropyrimidin-1(2H)-yl)-2-fluorophenyl)piperidin-4-yl)methyl acetate

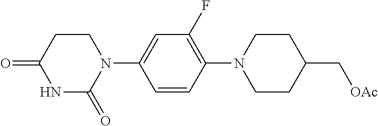 [SMILES: CC(=O)OCC1CCN(c2ccc(N3CCC(=O)NC3=O)cc2F)CC1]

A mixture of 3-((3-fluoro-4-(4-(hydroxymethyl)piperidin-1-yl)phenyl)amino) propanoic acid (55.0 g, crude) and urea (50.0 g, 833.3 mmol) in AcOH (600 mL) was stirred at 120° C. for 12 hours. The reaction mixture was cooled to rt and concentrated. The residue was poured into water (200 mL) and extracted with DCM (100 mL) for three times. The combined organic layers were washed with brine (100 mL), dried over Na₂SO₄, filtered and concentrated to give a residue which was purified by chromatography on silica gel column (DCM/MeOH=20/1) to afford the product (55 g, yield: 81%). [M+H]⁺=364.2.

### Step 5: 1-(3-fluoro-4-(4-(hydroxymethyl)piperidin-1-yl)phenyl)dihydropyrimidine-2,4(1H,3H)-dione

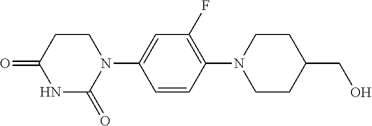 [SMILES: O=C1CCN(c2ccc(N3CCC(CO)CC3)c(F)c2)C(=O)N1]

A solution of (1-(4-(2,4-dioxotetrahydropyrimidin-1(2H)-yl)-2-fluorophenyl)piperidin-4-yl)methyl acetate (50.0 g, 137.7 mmol) in MeOH (600 mL) was added AcCl (12.0 g, 152.8 mmol) at rt. The reaction mixture was stirred at rt for 12 hours. Then the reaction mixture was filtered. The filtered cake was dissolved in water (50 mL) and adjusted pH=7 with aq Na₂CO₃. The mixture was filtered and the filtered cake was triturated with DCM (20 mL) and dried to afford product (26.084 g, yield: 59%). ¹H NMR (DMSO-d₆, 400 MHz): δ 10.36 (s, 1H), 7.17-7.14 (m, 1H), 7.07-7.00 (m, 2H), 4.48 (t, J=4.8 Hz, 1H), 3.73 (t, J=5.2 Hz, 2H), 3.33-3.29 (m, 4H), 2.70-2.60 (m, 4H), 1.77-1.74 (m, 2H), 1.50-1.46 (m, 1H), 1.34-1.27 (m, 2H). [M+H]⁺=322.2.

Step 6: 1-(4-(2,4-dioxotetrahydropyrimidin-1(2H)-yl)-2-fluorophenyl)piperidine-4-carbaldehyde

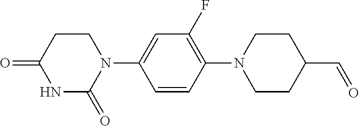 [SMILES: O=CC1CCN(c2ccc(N3CCC(=O)NC3=O)cc2F)CC1]

The titled compound was synthesized in a manner similar to that in Example 79 step 7 from 1-(3-fluoro-4-(4-(hydroxymethyl)piperidin-1-yl)phenyl)dihydropyrimidine-2,4(1H,3H)-dione and IBX. [M+H]⁺=320.2

### Step 7: 1-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)-3-fluorophenyl)dihydropyrimidine-2,4(1H,3H)-dione

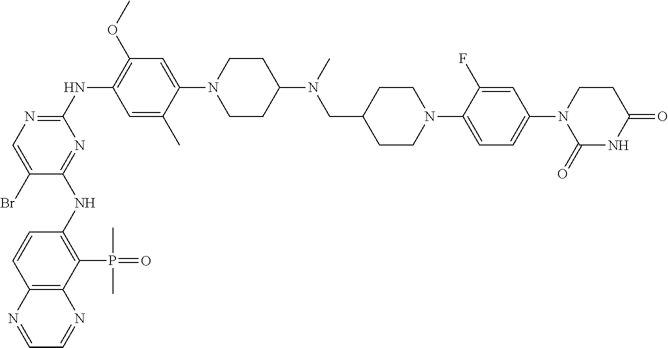 [SMILES: COc1cc(N2CCC(N(C)CC3CCN(c4ccc(N5CCC(=O)NC5=O)cc4F)CC3)CC2)c(C)cc1Nc1ncc(Br)c(Nc2ccc3nccnc3c2P(C)(C)=O)n1]

The titled compound was synthesized in a manner similar to that in Example 79 step 8 1-(4-(2,4-dioxotetrahydropyrimidin-1(2H)-yl)-2-fluorophenyl)piperidine-4-carbaldehyde and (6-((5-bromo-2-((2-methoxy-5-methyl-4-(4-(methylamino)piperidin-1-yl)phenyl)amino)pyrimidin-4-yl)amino)quinoxalin-5-yl)dimethylphosphine oxide. ¹H NMR (400 MHz, DMSO) δ 12.69 (s, 1H), 10.36 (s, 1H), 8.86 (d, J=4.6 Hz, 3H), 8.26 (d, J=8.1 Hz, 2H), 7.93 (d, J=8.6 Hz, 1H), 7.36 (s, 1H), 7.17 (d, J=13.3 Hz, 1H), 7.05 (s, 2H), 6.76 (s, 1H), 3.83-3.67 (m, 5H), 3.35 (s, 2H), 3.09 (s, 2H), 2.68 (d, J=6.3 Hz, 7H), 2.34 (s, 2H), 2.27 (s, 3H), 2.10 (s, 3H), 2.03 (d, J=14.4 Hz, 6H), 1.83 (d, J=11.6 Hz, 4H), 1.63 (d, J=12.2 Hz, 3H), 1.28 (d, J=11.3 Hz, 2H); [M+H]⁺=928.

### Example 83:1-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)-2-methylphenyl)dihydropyrimidine-2,4(1H,3H)-dione

### Step 1: (1-(3-methyl-4-nitrophenyl)piperidin-4-yl)methanol

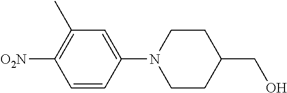 [SMILES: Cc1cc(N2CCC(CO)CC2)ccc1[N+](=O)[O-]]

To a solution of 4-fluoro-2-methyl-1-nitrobenzene (31 g, 200 mmol) and 4-piperidinemethanol (28 g, 240 mmol) in DMF (200 mL) was added K₂CO₃ (55 g, 400 mmol) at 25° C. The mixture reaction was stirred at 80° C. for 15 h. The reaction was cooled to room temperature, the mixture was poured into ice-water (2000 mL) and stirred for 20 mins. The solid was filtered and washed with water (100 mL×2), dried to give the product (50 g, 100%). [M+H]⁺=251.2.

### Step 2: (1-(4-amino-3-methylphenyl)piperidin-4-yl)methanol

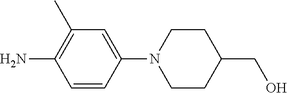 [SMILES: Cc1cc(N2CCC(CO)CC2)ccc1N]

Under N₂, to a solution of (1-(3-methyl-4-nitrophenyl)piperidin-4-yl)methanol (50 g, 200 mmol) in MeOH (500 mL) was added 10% Pd/C (5 g) at 25° C. And then the mixture was exchanged with H₂ two times and stirred under H₂ atmosphere at 25° C. for 15 h. The mixture was filtered through a pad of Celite and washed with MeOH (30 mL). The filtrate was concentrated under vacuum to obtain the product (41 g, 93.0%). [M+H]⁺=221.2.

### Step 3: (1-(4-(2,4-dioxotetrahydropyrimidin-1(2H)-yl)-3-methylphenyl)piperidin-4-yl)methyl acetate

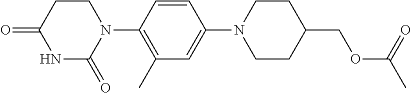 [SMILES: CC(=O)OCC1CCN(c2ccc(N3CCC(=O)NC3=O)c(C)c2)CC1]

To a solution of (1-(4-amino-3-methylphenyl)piperidin-4-yl)methanol (41 g, 186 mmol) in toluene (200 mL) was added acrylic acid (41 g, 0.57 mol) at 25° C. The mixture was stirred at 80° C. for 2 h. The reaction was cooled to 25° C., then HOAc (200 mL) and urea (90 g, 1.5 mol) were added. The mixture was stirred at 110° C. for 24 h. The reaction was cooled to 25° C. and concentrated under vacuum. To the residue was added Na₂CO₃ (aqueous, saturated, 300 m1) and EtOAc(300 m1). The solid was filtered and dried to give the product (24 g, 36%). [M+H]⁺=360.2.

### Step 4: 1-(4-(4-(hydroxymethyl)piperidin-1-yl)-2-methylphenyl)dihydropyrimidine-2.4(1H,3H)-dione

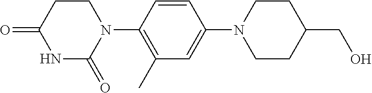 [SMILES: Cc1cc(N2CCC(CO)CC2)ccc1N1CCC(=O)NC1=O]

(1-(4-(2,4-dioxotetrahydropyrimidin-1(2H)-yl)-3-methylphenyl)piperidin-4-yl)methyl acetate(24 g, 67 mmol) was added into 4N HCl (250 mL) at 25° C. The mixture was stirred at 100° C. for 30 min. The reaction was cooled to 10° C. and then adjusted to pH=7 with sat. NaHCO₃. The solid was collected by filtrated, washed by water (50.0 mL), and dried to obtain the product (13 g, 61%). [M+H]⁺=318.2.

### Step 5: 1-(4-(2,4-dioxotetrahydropyrimidin-1(2H)-yl)-3-methylphenyl)piperidine-4-carbaldehyde

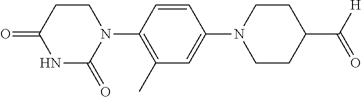 [SMILES: Cc1cc(N2CCC(C=O)CC2)ccc1N1CCC(=O)NC1=O]

To a solution of 1-(4-(4-(hydroxymethyl)piperidin-1-yl)-2-methylphenyl)dihydropyrimidine-2,4(1H,3H)-dione (10 g, 31 mmol) in DMSO (50 mL) was added IBX (18 g, 63 mmol) in portions at 25° C. The mixture was stirred at 25° C. for 15 h. Water (300 mL) was added to the reaction at 25° C. The solid was filtered and washed with water (100 mL) and then EtOAc (100.0 mL). The resulting solution was extracted with 4×200 mL of EtOAc. The combined organic layer was dried over Na₂SO₄ and concentrated under vacuum to afford a crude residue. The crude product was purified by column chromatography to afford the product (5 g, 50%). [M+H]⁺=316.

### Step 6: 1-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)-2-methylphenyl)dihydropyrimidine-2,4(1H,3H)-dione

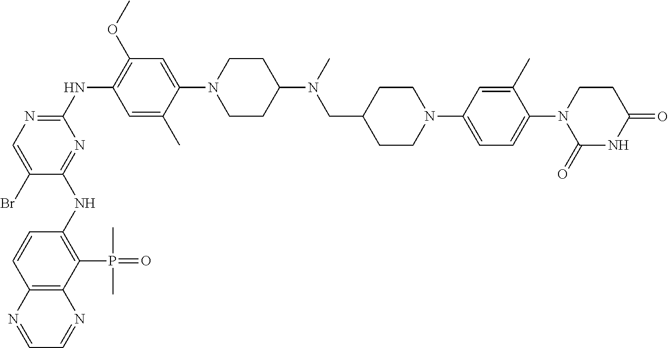 [SMILES: COc1cc(N2CCC(N(C)CC3CCN(c4ccc(N5CCC(=O)NC5=O)c(C)c4)CC3)CC2)c(C)cc1Nc1ncc(Br)c(Nc2ccc3nccnc3c2P(C)(C)=O)n1]

The titled compound was synthesized in a manner similar to that in Example 79 step 8. ¹H NMR (400 MHz, DMSO) δ 12.67 (s, 1H), 10.24 (s, 1H), 8.84 (dd, J=8.0, 1.9 Hz, 3H), 8.25 (d, J=4.5 Hz, 2H), 7.90 (d, J=8.7 Hz, 1H), 7.32 (s, 1H), 7.02 (d, J=8.6 Hz, 1H), 6.84-6.64 (m, 3H), 3.75 (s, 3H), 3.68 (d, J=9.4 Hz, 3H), 3.50-3.39 (m, 1H), 3.06 (s, 2H), 2.65 (dd, J=13.1, 9.2 Hz, 5H), 2.45-2.38 (m, 1H), 2.34-2.19 (m, 7H), 2.09 (d, J=7.8 Hz, 5H), 2.00 (d, J=14.4 Hz, 6H), 1.78 (s, 4H), 1.58-1.64 (m, 3H), 1.10-1.20 (m, 2H); [M+H]⁺=924.

### Example 84: 1-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)-3-methylphenyl)dihydropyrimidine-2,4(1H,3H)-dione

### Step 1: (1-(2-methyl-4-nitrophenyl)piperidin-4-yl)methanol

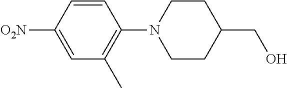 [SMILES: Cc1cc([N+](=O)[O-])ccc1N1CCC(CO)CC1]

A mixture of 1-fluoro-2-methyl-4-nitrobenzene (100.0 g, 645.2 mmol), piperidin-4-ylmethanol (111.3 g, 967.8 mmol) and DIEA (166.0 g, 1286.8 mmol) in THF (1.5 L) and DMF (50 mL) was stirred at 50° C. overnight. The reaction mixture was cooled down to rt, poured into water (1 L) and extracted with EA (500 mL) for three times. The combined organic layers were washed with brine (500 mL), dried over Na₂SO₄, filtered and concentrated. The residue was slurried with Et₂O (100 mL) and dried to afford the product (75 g, yield: 46%). [M+H]⁺=251.1.

### Step 2: (1-(4-amino-2-methylphenyl)piperidin-4-yl)methanol

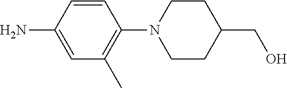 [SMILES: Cc1cc(N)ccc1N1CCC(CO)CC1]

To a solution of (1-(2-methyl-4-nitrophenyl)piperidin-4-yl)methanol (75.0 g, 297.6 mmol) in EtOH (750 mL) was added Pd/C (7.5 g) at rt. The reaction mixture was stirred at 60° C. overnight under H₂ (1 atm). The reaction mixture was cooled to rt, filtered and concentrated to afford the product (65 g, yield: 99%). [M+H]⁺=221.2.

### Step 3: 3-((4-(4-(hydroxymethyl)piperidin-1-yl)-3-methylphenyl)amino)propanoic acid

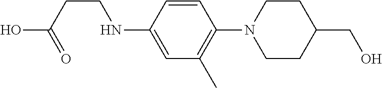 [SMILES: Cc1cc(NCCC(=O)O)ccc1N1CCC(CO)CC1]

A mixture of (1-(4-amino-2-methylphenyl)piperidin-4-yl)methanol (22.0 g, 99.1 mmol) and acrylic acid (22.0 g, 3055.6 mmol) in toluene (300 mL) was stirred at 100° C. for 1.5 hours. The reaction mixture was cooled down to rt and filtered. The filtered cake was dried to afford the product (27.8 g, yield: 96%). [M+H]⁺=293.2.

### Step 4: (1-(4-(2,4-dioxotetrahydropyrimidin-1(2H)-yl)-2-methylphenyl)piperidin-4-yl)methyl acetate

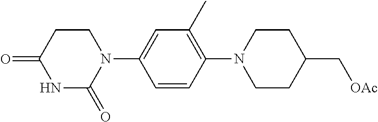 [SMILES: CC(=O)OCC1CCN(c2ccc(N3CCC(=O)NC3=O)cc2C)CC1]

A mixture of 3-((4-(4-(hydroxymethyl)piperidin-1-yl)-3-methylphenyl)amino)propanoic acid (27.8 g, 95.1 mmol) and urea (18.0 g, 300.0 mmol) in AcOH (500 mL) was stirred at 120° C. overnight. The reaction mixture was cooled to rt and concentrated. The residue was poured into water (200 mL) and extracted with DCM (100 mL) for three times. The combined organic layers were washed with brine (100 mL), dried over Na₂SO₄, filtered and concentrated to give a residue which was purified by chromatography on silica gel column (DCM/MeOH=20/1) to afford the product (19.1 g, yield: 56%). [M+H]⁺=360.2.

### Step 5: 1-(4-(4-(hydroxymethyl)piperidin-1-yl)-3-methylphenyl)dihydropyrimidine-2.4(1H,3H)-dione

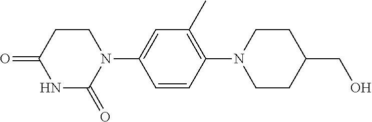 [SMILES: Cc1cc(N2CCC(=O)NC2=O)ccc1N1CCC(CO)CC1]

To a solution of (1-(4-(2,4-dioxotetrahydropyrimidin-1(2H)-yl)-2-methylphenyl)piperidin-4-yl)methyl acetate (19.1 g, 53.2 mmol) in MeOH (500 mL) was added AcCl (8.4 g, 106.4 mmol) at rt. The reaction mixture was stirred at rt overnight. Then the reaction mixture was filtered. The filtered cake was dissolved in water (50 mL) and adjusted pH=7 with sat. Na₂CO₃. The mixture was filtered and the filtered cake was slurried with acetonitrile (20 mL) and dried to afford the product (10.96 g, yield: 65%). ¹H NMR (DMSO-d₆, 400 MHz): δ 10.28 (s, 1H), 7.10-6.99 (m, 3H), 4.48 (t, J=4.8 Hz, 1H), 3.71 (t, J=5.2 Hz, 2H), 3.33-3.31 (m, 2H), 3.04 (d, J=11.2 Hz, 2H), 2.67 (t, J=5.2 Hz, 2H), 2.59-2.53 (m, 2H), 2.22 (s, 3H), 1.77-1.74 (m, 2H), 1.52-1.44 (m, 1H), 1.34-1.24 (m, 2H). [M+H]⁺=318.1.

### Step 6: 1-(4-(2,4-dioxotetrahydropyrimidin-1(2H)-yl)-2-methylphenyl)piperidine-4-carbaldehyde

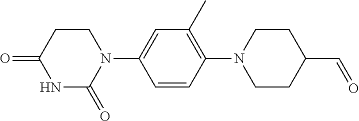 [SMILES: Cc1cc(N2CCC(=O)NC2=O)ccc1N1CCC(C=O)CC1]

The titled compound was synthesized in a manner similar to that in Example 79 step 7 from 1-(4-(4-(hydroxymethyl)piperidin-1-yl)-3-methylphenyl)dihydropyrimidine-2,4(1H,3H)-dione and IBX. [M+H]⁺=316.2.

### Step 7: 1-(4-(4-(((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)methyl)piperidin-1-yl)-3-methylphenyl)dihydropyrimidine-2,4(1H,3H)-dione

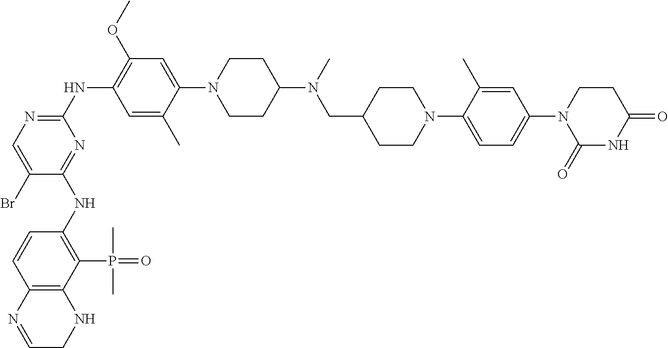 [SMILES: COc1cc(N2CCC(N(C)CC3CCN(c4ccc(N5CCC(=O)NC5=O)cc4C)CC3)CC2)c(C)cc1Nc1ncc(Br)c(Nc2ccc3c(c2P(C)(C)=O)NCC=N3)n1]

The titled compound was synthesized in a manner similar to that in Example 79 step 8. ¹H NMR (400 MHz, DMSO) δ 12.68 (s, 1H), 10.29 (s, 1H), 8.85 (dd, J=6.8, 2.0 Hz, 3H), 8.25 (s, 2H), 7.92 (s, 1H), 7.33 (s, 1H), 7.15-6.92 (m, 3H), 6.75 (s, 1H), 3.76 (s, 3H), 3.69 (t, J=6.7 Hz, 2H), 3.02 (m, 4H), 2.66 (m, 6H), 2.32 (s, 3H), 2.23 (m, 6H), 2.09 (s, 3H), 2.01 (d, J=14.4 Hz, 6H), 1.80 (m, 4H), 1.62 (m, 3H), 1.27 (m, 3H); [M+H]⁺=926.

### Example 85: 1-(4-(3-((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)propoxy)phenyl)dihydropyrimidine-2,4(1H,3H)-dione

### Step 1: 3,3′-((4-hydroxyphenyl)azanediyl)dipropionic acid

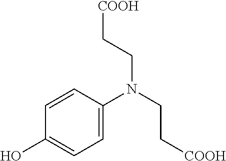 [SMILES: O=C(O)CCN(CCC(=O)O)c1ccc(O)cc1]

To a solution of 4-aminophenol (250 g, 2.29 mol) and acrylic acid (363 g, 5.04 mol) in the water (1.25 L) was stirred at 80˜85° C. for 16 hrs. The mixture was cooled to 20-25° C. The reaction mixture was filtered and the filter cake was washed with H₂O (2.00 L). The filter cake was concentrated in vacuum to afford the product (552 g, 95.1% yield). ¹H NMR (400 MHz, DMSO) δ_{H} 12.18 (br s, 2H), 8.67 (br s, 1H), 6.48-6.88 (m, 4H), 3.38 (s, 5H), 2.36 (t, J=7.06 Hz, 4H).

### Step 2: 1-(4-hydroxyphenyl)dihydropyrimidine-2.4(1H,3H)-dione

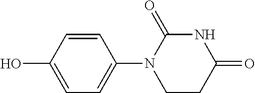 [SMILES: O=C1CCN(c2ccc(O)cc2)C(=O)N1]

To a solution of 3,3′-((4-hydroxyphenyl)azanediyl)dipropionic acid (300 g, 1.18 mol) in the AcOH (1.50 L) was added urea (106 g, 1.76 mol) at 20˜30° C. After addition, the mixture was stirred at 120˜130° C. for 12 hrs. 10.0% HCl (1.00 L) was added to the reactor at 120˜130° C. After addition, the mixture was stirred at 120˜130° C. for 1 hr. The reaction mixture was cooled to 20° C., filtered and the filter cake was rinsed with petroleum ether (2.00 L) to afford the crude product (185 g). ¹H NMR (400 MHz, DMSO) δ_{H} 10.3 (s, 1H), 9.45 (s, 1H), 7.00-7.26 (m, 2H), 6.67-6.86 (m, 2H), 3.67 (t, J=6.72 Hz, 2H), 2.67 (t, J=6.72 Hz, 2H).

### Step 3: 1-(4-((tert-butyldimethylsilyl)oxy)phenyl)dihydropyrimidine-2,4(1H,3H)-dione

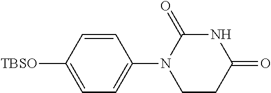 [SMILES: CC(C)(C)[Si](C)(C)Oc1ccc(N2CCC(=O)NC2=O)cc1]

To a solution of 1-(4-hydroxyphenyl)dihydropyrimidine-2,4(1H,3H)-dione (80.0 g, crude) and imidazole (52.8 g, 0.770 mol) in the DCM (400 mL) was added TBSCl (64.3 g, 0.420 mol) at 10˜15° C. Then the mixture was stirred at 25° C. for 12 hrs. The reaction mixture was partitioned between DCM (400 mL) and water (500 mL). The aqueous layer was extracted with DCM (200 mL). The combined organic layers were washed with brine (200 mL) and dried with anhydrous Na₂SO₄. The solvent was removed to afford the product (120 g, 96.1% yield). ¹H NMR (400 MHz, DMSO) δ_{H} 10.31 (s, 1H), 7.07-7.40 (m, 2H), 6.67-6.95 (m, 2H), 3.72 (t, J=6.73 Hz, 2H), 2.68 (t, J=6.73 Hz, 2H), 0.95 (s, 9H), 0.20 (s, 6H).

### Step 4: 1-(4-((tert-butyldimethylsilyl)oxy)phenyl)-3-((2-(trimethylsilyl)ethoxy)methyl)dihydropyrimidine-2,4(1H,3H)-dione

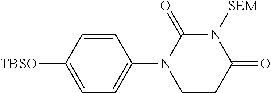 [SMILES: CC(C)(C)[Si](C)(C)Oc1ccc(N2CCC(=O)N(COCC[Si](C)(C)C)C2=O)cc1]

To a solution of 1-(4-((tert-butyldimethylsilyl)oxy)phenyl)dihydropyrimidine-2,4(1H,3H)-dione (85.0 g, 0.265 mol) and DIEA (137 g, 1.06 mol) in the ACN (425 mL) was added SEMCl (88.4 g, 0.530 mol) at 25° C. The mixture was stirred at 85° C. for 12 hrs. TLC indicated starting material was consumed. The reaction mixture was cooled to 25° C. and put into water (500 mL). The mixture was filtered and concentrated in reduced pressure at 45° C. The residue was purified by activated carbon, then purified by silica gel to give product (81.8 g, 68.6% yield). ¹H NMR (400 MHz, DMSO) δ_{H} 7.03-7.21 (m, 2H), 6.85 (d, J=8.60 Hz, 2H), 5.29 (s, 2H), 3.78 (t, J=6.62 Hz, 2H), 3.61-3.72 (m, 2H), 2.89 (t, J=6.62 Hz, 2H), 0.98 (s, 9H), 0.72-0.93 (m, 1H), 0.20 (s, 5H), 0.04-0.04 (m, 10H).

### Step 5: 1-(4-hydroxyphenyl)-3-((2-(trimethylsilyl)ethoxy)methyl)dihydropyrimidine-2,4(1H,3H)-dione

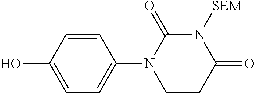 [SMILES: C[Si](C)(C)CCOCN1C(=O)CCN(c2ccc(O)cc2)C1=O]

To a solution of 1-(4-((tert-butyldimethylsilyl)oxy)phenyl)-3-((2-(trimethylsilyl)ethoxy)methyl)dihydropyrimidine-2,4(1H,3H)-dione (81.8 g, 0.182 mol) and NH₄F (13.5 g, 0.364 mol) in the MeOH (620 mL) was stirred at 20° C. for 1 hr. TLC indicated starting material was consumed. The resulting solution was filtered and the filtrate was concentrated to afford the crude residue. The crude product was triturated with petroleum ether: ethyl acetate=10: 1 (200 mL) at 15° C. for 45 mins to afford the product (41.9 g, 68.6% yield). ¹H NMR (400 MHz, CDCl₃) δ_{H} 6.96-7.15 (m, 2H), 6.63-6.84 (m, 2H), 5.31 (s, 2H), 5.93 (s, 1H), 3.76 (t, J=6.80 Hz, 2H), 3.66-3.73 (m, 2H), 2.90 (t, J=6.58 Hz, 2 H), 0.87-1.16 (m, 2H), 0.02 (s, 9H).

### Step 6: 1-(4-(3-iodopropoxy)phenyl)-3-((2-(trimethylsilyl)ethoxy)methyl)dihydropyrimidine-2,4(1H,3H)-dione

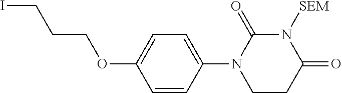 [SMILES: C[Si](C)(C)CCOCN1C(=O)CCN(c2ccc(OCCCI)cc2)C1=O]

To a solution of PPh₃ (56.1 g, 214 mmol), 1-(4-hydroxyphenyl)-3-((2-(trimethylsilyl)ethoxy)methyl)dihydropyrimidine-2,4(1H,3H)-dione (41.9 g, 125 mmol), 3-iodopropan-1-ol (53.1 g, 285 mmol, 27.3 mL) in THF (250 mL) was added DIAD (43.2 g, 214 mmol), the reaction was stirred at 15° C. for 3 hrs. TLC showed the staring material was consumed completely and the product was formed. The mixture was concentrated in vacuum. The crude product was purified by silica gel chromatography eluted with Petroleum ether in Ethyl acetate=100˜0% to give the product (25.0 g, 39.7% yield). [M+Na]⁺=527.

### Step 6: 1-(4-(3-((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)propoxy)phenyl)-3-((2-(trimethylsilyl)ethoxy)methyl)dihydropyrimidine-2,4(1H,3H)-dione

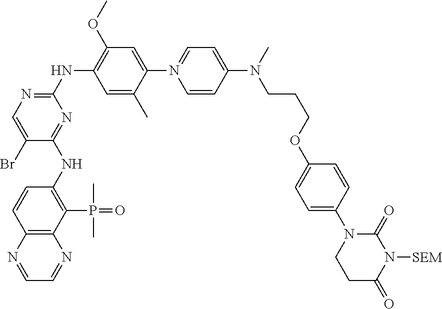

The titled compound was prepared in a manner similar to that in Example 18 step 7. [M+H]⁺=1001.

### Step 7: 1-(4-(3-((1-(4-((5-bromo-4-((5-(dimethylphosphoryl)quinoxalin-6-yl)amino)pyrimidin-2-yl)amino)-5-methoxy-2-methylphenyl)piperidin-4-yl)(methyl)amino)propoxy)phenyl)dihydropyrimidine-2,4(1H,3H)-dione

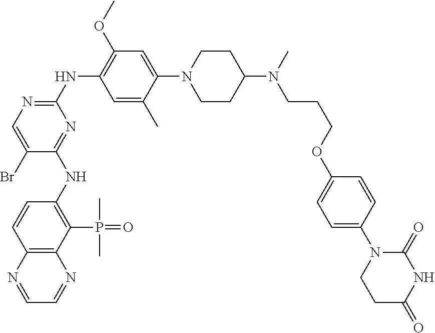 [SMILES: COc1cc(N2CCC(N(C)CCCOc3ccc(N4CCC(=O)NC4=O)cc3)CC2)c(C)cc1Nc1ncc(Br)c(Nc2ccc3nccnc3c2P(C)(C)=O)n1]

The titled compound was prepared in a manner similar to that in Example 18 step 8.

¹H NMR (400 MHz, DMSO) δ 12.69 (s, 1H), 10.32 (s, 1H), 8.86 (d, J=9.4 Hz, 3H), 8.27 (s, 2H), 7.94 (s, 1H), 7.34 (s, 1H), 7.23 (d, J=8.7 Hz, 2H), 6.95 (d, J=8.6 Hz, 2H), 6.75 (s, 1H), 4.03 (s, 2H), 3.77 (s, 3H), 3.71 (s, 2H), 3.07 (s, 2H), 2.65 (d, J=29.3 Hz, 6H), 2.26 (s, 3H), 2.09 (s, 3H), 2.02 (d, J=14.3 Hz, 6H), 1.86 (s, 2H), 1.77 (s, 2H), 1.63 (s, 2H), 1.24 (s, 1H); [M+H]⁺=871.

### Example 86: 3-(4-(2-(4-(4-((5-bromo-4-((5-(dimethylphosphoryl)quinoxalin-6-yl)amino)pyrimidin-2-yl)amino)-5-methoxy-2-methylphenyl)piperazin-1-yl)ethyl)phenyl)piperidine-2,6-dione

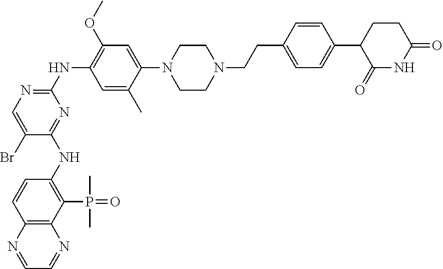 [SMILES: COc1cc(N2CCN(CCc3ccc(C4CCC(=O)NC4=O)cc3)CC2)c(C)cc1Nc1ncc(Br)c(Nc2ccc3nccnc3c2P(C)(C)=O)n1]

The titled compound was synthesized in the procedures similar to Example 1. ¹HNMR (400 MHz, DMSO) δ 12.69 (s, 1H), 10.84 (s, 1H), 8.86 (s, 3H), 8.28 (d, J=12.1 Hz, 2H), 7.95 (s, 1H), 7.34 (s, 1H), 7.23 (s, 2H), 7.16 (s, 2H), 6.80 (s, 1H), 3.79 (s, 4H), 2.90 (s, 5H), 2.79 (s, 3H), 2.65 (d, J=16.2 Hz, 8H), 2.17 (s, 1H), 2.11 (s, 3H), 2.02 (d, J=14.1 Hz, 6H); [M+H]⁺=812.

### Example 87: 3-(6-(2-(4-(4-((5-bromo-4-((5-(dimethylphosphoryl)quinoxalin-6-yl)amino)pyrimidin-2-yl)amino)-5-methoxy-2-methylphenyl)piperazin-1-yl)ethyl)pyridin-3-yl)piperidine-2,6-dione

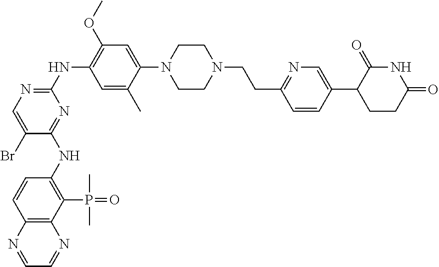 [SMILES: COc1cc(N2CCN(CCc3ccc(C4CCC(=O)NC4=O)cn3)CC2)c(C)cc1Nc1ncc(Br)c(Nc2ccc3nccnc3c2P(C)(C)=O)n1]

The titled compound was synthesized in the procedures similar to Example 13. ¹H NMR (400 MHz, DMSO) δ 12.69 (s, 1H), 10.91 (s, 1H), 8.87 (s, 3H), 8.37 (s, 1H), 8.28 (d, J=9.4 Hz, 2H), 7.94 (d, J=8.5 Hz, 1H), 7.59 (d, J=7.9 Hz, 1H), 7.40-7.20 (m, 2H), 6.80 (s, 1H), 3.92 (d, J=12.0 Hz, 1H), 3.78 (s, 3H), 2.92 (d, J=19.0 Hz, 6H), 2.82-2.55 (m, 8H), 2.37-2.19 (m, 1H), 2.11 (s, 3H), 2.03 (d, J=13.7 Hz, 7H); [M+H]⁺=813.

### Example 88: 3-(4-(4-((4-(4-((5-bromo-4-((5-(dimethylphosphoryl)quinoxalin-6-yl)amino)pyrimidin-2-yl)amino)-5-methoxy-2-methylphenyl)piperazin-1-yl)methyl)piperidin-1-yl)phenyl)piperidine-2,6-dione

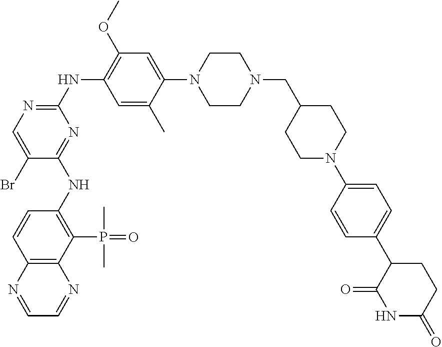 [SMILES: COc1cc(N2CCN(CC3CCN(c4ccc(C5CCC(=O)NC5=O)cc4)CC3)CC2)c(C)cc1Nc1ncc(Br)c(Nc2ccc3nccnc3c2P(C)(C)=O)n1]

The titled compound was synthesized in the procedures similar to Example 22. ¹H NMR (400 MHz, DMSO) δ 12.69 (s, 1H), 10.79 (s, 1H), 8.86 (d, J=5.2 Hz, 3H), 8.27 (s, 2H), 7.94 (d, J=8.9 Hz, 1H), 7.35 (s, 1H), 7.04 (d, J=7.9 Hz, 2H), 6.90 (d, J=7.5 Hz, 2H), 6.81 (s, 1H), 3.79 (s, 3H), 3.68 (d, J=13.6 Hz, 3H), 2.90 (s, 4H), 2.72-2.55 (m, 5H), 2.45 (s, 2H), 2.26 (s, 2H), 2.19-1.95 (m, 11H), 1.83 (d, J=11.7 Hz, 2H), 1.71 (s, 1H), 1.24 (d, J=8.7 Hz, 2H); [M+H]⁺=881.

### Example 89: 3-(4-(4-((4-(4-((5-bromo-4-((5-(dimethylphosphoryl)quinoxalin-6-yl)amino)pyrimidin-2-yl)amino)-3-methoxyphenyl)piperazin-1-yl)methyl)piperidin-1-yl)phenyl)piperidine-2,6-dione

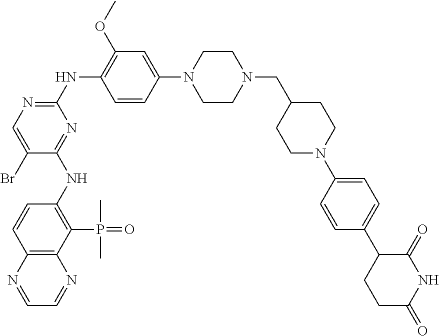 [SMILES: COc1cc(N2CCN(CC3CCN(c4ccc(C5CCC(=O)NC5=O)cc4)CC3)CC2)ccc1Nc1ncc(Br)c(Nc2ccc3nccnc3c2P(C)(C)=O)n1]

The titled compound was synthesized in the procedures similar to Example 22.1H NMR (400 MHz, DMSO) δ 12.70 (s, 1H), 10.78 (s, 1H), 8.86 (s, 3H), 8.23 (s, 2H), 7.95 (s, 1H), 7.32 (s, 1H), 7.04 (d, J=7.1 Hz, 2H), 6.91 (s, 2H), 6.69 (s, 1H), 6.50 (s, 1H), 3.78 (s, 3H), 3.70 (s, 3H), 3.18 (s, 4H), 3.04-2.87 (m, 1H), 2.66 (s, 6H), 2.47-2.40 (m, 1H), 2.24 (m, 2H), 2.12 (m, 1H), 2.02 (d, J=14.1 Hz, 7H), 1.89-1.68 (m, 3H), 1.19-1.25 (m, 2H); [M+H]⁺=867.

### Example 90: 3-(4-(4-(2-(4-(4-((5-bromo-4-((5-(dimethylphosphoryl)quinoxalin-6-yl)amino)pyrimidin-2-yl)amino)-5-methoxy-2-methylphenyl)piperazin-1-yl)ethyl)piperidin-1-yl)phenyl)piperidine-2,6-dione

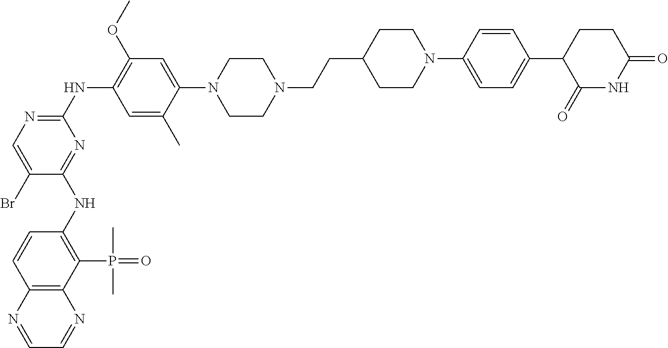 [SMILES: COc1cc(N2CCN(CCC3CCN(c4ccc(C5CCC(=O)NC5=O)cc4)CC3)CC2)c(C)cc1Nc1ncc(Br)c(Nc2ccc3nccnc3c2P(C)(C)=O)n1]

The titled compound was synthesized in the procedures similar to Example 23. ¹H NMR (400 MHz, DMSO) δ 12.69 (s, 1H), 10.78 (s, 1H), 8.86 (d, J=6.6 Hz, 3H), 8.27 (s, 2H), 7.94 (s, 1H), 7.35 (s, 1H), 7.02 (s, 2H), 6.89 (d, J=7.7 Hz, 2H), 6.79 (s, 1H), 3.60-3.88 (m, 6H), 2.84-2.90 (m, 4H), 2.63 (t, J=12.2 Hz, 3H), 2.40-2.45 (m, 4H), 2.02-2.15 (m, 12H), 1.78 (d, J=10.5 Hz, 3H), 1.46 (s, 3H), 1.26-1.30 (m, 3H); [M+H]⁺=895.

### Example 91: 3-(4-(2-(4-(4-((5-bromo-4-((5-(dimethylphosphoryl)quinoxalin-6-yl)amino)pyrimidin-2-yl)amino)-5-methoxy-2-methylphenyl)piperazin-1-yl)ethoxy)phenyl)piperidine-2,6-dione

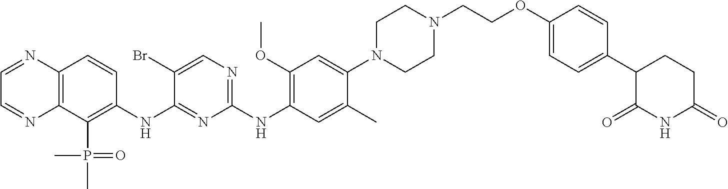 [SMILES: COc1cc(N2CCN(CCOc3ccc(C4CCC(=O)NC4=O)cc3)CC2)c(C)cc1Nc1ncc(Br)c(Nc2ccc3nccnc3c2P(C)(C)=O)n1]

The titled compound was synthesized in the procedures similar to Example 18. ¹H NMR (400 MHz, DMSO) δ 12.69 (s, 1H), 10.81 (s, 1H), 8.86 (d, J=8.0 Hz, 3H), 8.28 (d, J=10.1 Hz, 2H), 7.93 (d, J=8.7 Hz, 1H), 7.35 (s, 1H), 7.14 (d, J=8.4 Hz, 2H), 6.94 (d, J=8.5 Hz, 2H), 6.80 (s, 1H), 4.13 (s, 2H), 3.78 (s, 4H), 2.90 (s, 4H), 2.79 (s, 2H), 2.68 (s, 5H), 2.40-2.50 (m,2 H)2.17 (d, J=11.5 Hz, 1H), 2.10 (s, 3H), 2.02 (d, J=14.3 Hz, 6H); [M+H]⁺=828.

### Example 92: 3-(4-(3-(4-(4-((5-bromo-4-((5-(dimethylphosphoryl)quinoxalin-6-yl)amino)pyrimidin-2-yl)amino)-5-methoxy-2-methylphenyl)piperazin-1-yl)propoxy)phenoxy)piperidine-2,6-dione

### Step 1: 4-(3-((tert-butyldimethylsilyl)oxy)propoxy)phenol

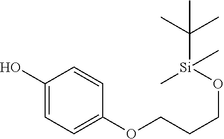 [SMILES: CC(C)(C)[Si](C)(C)OCCCOc1ccc(O)cc1]

Hydroquinone (10.6 g, 96.1 mmol) was dissolved in DMF (100 mL). The solution was added (3-bromopropoxy)(tert-butyl)dimethylsilane (23.0 g, 91 mmol) and Cs₂CO₃ (45.0 g, 138.1 mmol). The mixture was stirred at 50° C. for 2h. The mixture was diluted with water (100 mL), extracted with EA (150 mL×2). The combined organic lays were washed with water (50 mL×3) and brine (50 mL×2), dried over Na₂SO₄ and concentrated under reduced pressure, the residue was purified by column chromatography to obtain the product (8.5 g, 31.3%). ¹H NMR (400 MHz, CDCl₃) δ_{H} 6.80-6.74 (m, 4H), 4.50 (s, 1H), 4.00 (t, J=6.4 Hz, 2H), 3.79 (t, J=6.4 Hz, 2H), 1.96 (t, J=6.0 Hz, 2H), 0.89 (s, 9H).

### Step 2: 3-bromopiperidine-2,6-dione

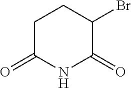

Br₂ (25.4 g, 158 mmol) was added to a solution of piperidine-2,6-dione (15.0 g, 132 mmol) in CHCl₃ (30 mL), the mixture is stirred for 4 h at 110° C. After cooling, the mixture was added water (200 mL), extracted with EA (200 mL×2). The combined organic lays were washed with water (100 mL) and brine (100 mL), dried over Na₂SO₄ and concentrated under reduced pressure. The residue was purified by column chromatography to give the product (7.9 g, 31%). [M+H]⁺=192.

### Step 3: 3-(4-(3-((tert-butyldimethylsilyl)oxy)propoxy)phenoxy) piperidine-2,6-dione

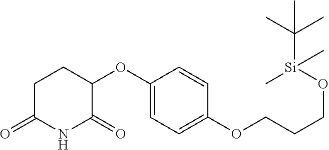 [SMILES: CC(C)(C)[Si](C)(C)OCCCOc1ccc(OC2CCC(=O)NC2=O)cc1]

4-(3-((Tert-butyldimethylsilyl)oxy)propoxy)phenol (22.1 g, 78.1 mmol) was dissolved in THF (100 mL). The solution was added NaH (4.7 g, 60%, 117.2 mmol) at 0° C., the resulting mixture was stirred for 1h. Then the mixture was dropped into a solution of 3-bromopiperidine-2,6-dione (15.0 g, 78.1 mmol) in THF (100 mL). The mixture was stirred at 60° C. for 2 h, then added saturated aqueous solution of NH₄Cl (100 mL) at 0° C., extracted with EA (100 mL×4). The combined organic phases were washed with water (50 mL×2) and brine (50 mL×2), dried over Na₂SO₄ and concentrated under reduced pressure. The residue was purified by column chromatography to give the product (14.5 g, 47%). ¹H NMR (400 MHz, CDCl3) δ_{H} 7.85 (s, 1H), 6.99-6.96 (m, 2H), 6.85-6.83 (m, 2H), 4.71-4.74 (dd, J₁=12.0, 3.3 Hz, 1H), 4.01 (t, J=6.4 Hz, 2H), 3.79 (t, J=6.0 Hz, 2H), 2.90-2.98 (m, 1H), 2.69-2.61 (m, 1H), 2.30-2.04 (m, 2H), 1.97-1.93 (m, 2H), 0.89 (s, 9H); [M+H]⁺=394.2.

### Step 4: 3-(4-(3-hydroxypropoxy)phenoxy)piperidine-2,6-dione

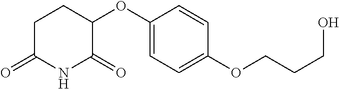 [SMILES: O=C1CCC(Oc2ccc(OCCCO)cc2)C(=O)N1]

3-(4-(3-((Tert-butyldimethylsilyl)oxy)propoxy)phenoxy)piperidine-2,6-dione (19.0 g, 48.3 mmol) was dissolved in THF (200 mL), was added TBAF (1M in THF) (72.5 mL, 72.5 mmol), the mixture was stirred at 25° C. for 5 h. The mixture was added water (100 mL), extracted with EA (150 mL×2). The combined organic phases were washed with water (100 mL) and brine (100 mL), dried over Na₂SO₄ and concentrated under reduced pressure. The residue was purified by column chromatography) to give the product (5.3 g, 39.3%). ¹H NMR (400 MHz, DMSO) δ_{H} 10.89 (s, 1H), 6.94 (d, J=9.1 Hz, 2H), 6.84 (d, J=9.1 Hz, 2H), 5.02 (dd, J=10.5, 4.9 Hz, 1H), 4.51 (t, J=5.1 Hz, 1H), 3.97 (t, J=6.4 Hz, 2H), 3.56-3.52 (m, 2H), 2.76-2.54 (m, 2H), 2.23-2.02 (m, 2H), 1.88-1.79 (m, 2H). [M+H]⁺=280.2.

### Step 5: 3-(4-((2,6-dioxopiperidin-3-yl)oxy)phenoxy)propanal

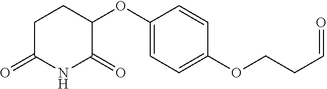 [SMILES: O=CCCOc1ccc(OC2CCC(=O)NC2=O)cc1]

The titled compound was prepared in a manner similar to that in Example 23 step 11. [M+H]⁺=278.

### Step 6: 3-(4-(3-(4-(4-((5-bromo-4-((5-(dimethylphosphoryl)quinoxalin-6-yl)amino)pyrimidin-2-yl)amino)-5-methoxy-2-methylphenyl)piperazin-1-yl)propoxy)phenoxy)piperidine-2,6-dione

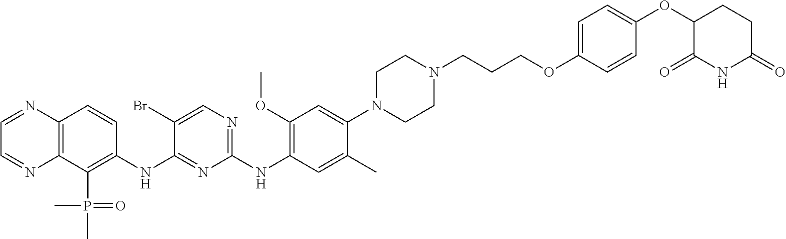 [SMILES: COc1cc(N2CCN(CCCOc3ccc(OC4CCC(=O)NC4=O)cc3)CC2)c(C)cc1Nc1ncc(Br)c(Nc2ccc3nccnc3c2P(C)(C)=O)n1]

The titled compound was prepared in a manner similar to that in Example 23 step 12. ¹H NMR (400 MHz, DMSO) δ 12.70 (s, 1H), 10.92 (s, 1H), 8.87 (d, J=11.4 Hz, 3H), 8.29 (d, J=7.7 Hz, 2H), 7.95 (d, J=8.8 Hz, 1H), 7.41 (s, 1H), 6.97 (d, J=8.0 Hz, 2H), 6.89 (d, J=7.3 Hz, 2H), 6.78 (s, 1H), 5.05 (s, 1H), 4.02 (s, 2H), 3.80 (s, 3H), 2.99 (s, 5H), 2.45-2.70 (m, 4H), 1.85-2.25 (m, 15H), 1.23 (s, 1H); [M+H]⁺=858. Example 93: 3-(4-(2-(4-(4-((5-bromo-4-((5-(dimethylphosphoryl)quinoxalin-6-yl)amino)pyrimidin-2-yl)amino)-5-methoxy-2-methylphenyl)piperazin-1-yl)ethyl)phenoxy)piperidine-2,6-dione

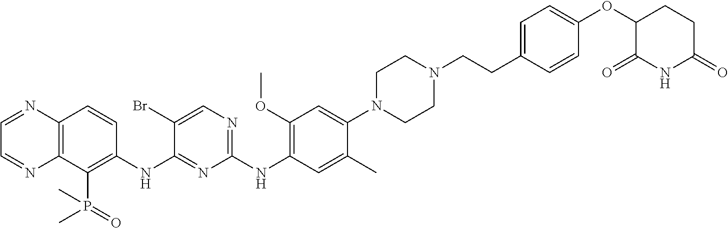 [SMILES: COc1cc(N2CCN(CCc3ccc(OC4CCC(=O)NC4=O)cc3)CC2)c(C)cc1Nc1ncc(Br)c(Nc2ccc3nccnc3c2P(C)(C)=O)n1]

The titled compound was synthesized in the procedures similar to Example 18. ¹H NMR (400 MHz, DMSO) δ 12.70 (s, 1H), 10.94 (s, 1H), 8.87 (d, J=8.3 Hz, 2H), 8.29 (d, J=11.5 Hz, 2H), 8.11 (d, J=17.5 Hz, 1H), 7.95 (s, 1H), 7.37 (s, 1H), 7.18 (s, 2H), 6.97 (s, 2H), 6.79 (s, 1H), 6.54 (s, 1H), 5.16 (s, 1H), 3.79 (s, 3H), 2.75 (dd, J=79.7, 43.5 Hz, 14H), 2.11 (s, 5H), 2.03 (d, J=14.3 Hz, 6H), 1.58 (d, J=13.2 Hz, 1H); [M+H]⁺=828.

### Example 94: 1-(4-(4-(2-(4-(4-((5-bromo-4-((5-(dimethylphosphoryl)quinoxalin-6-yl)amino)pyrimidin-2-yl)amino)-5-methoxy-2-methylphenyl)piperazin-1-yl)ethyl)piperidin-1-yl)phenyl)dihydropyrimidine-2,4(1H,3H)-dione

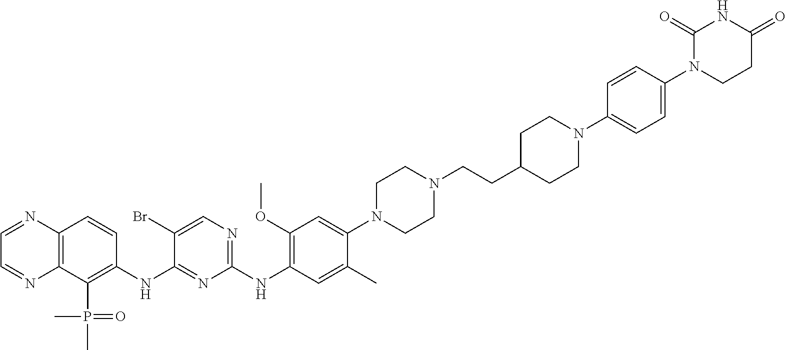 [SMILES: COc1cc(N2CCN(CCC3CCN(c4ccc(N5CCC(=O)NC5=O)cc4)CC3)CC2)c(C)cc1Nc1ncc(Br)c(Nc2ccc3nccnc3c2P(C)(C)=O)n1]

The titled compound was synthesized in the procedures similar to Example 55. ¹H NMR (400 MHz, DMSO) δ 12.69 (s, 1H), 10.27 (s, 1H), 8.86 (d, J=7.9 Hz, 3H), 8.26 (t, J=13.1 Hz, 2H), 7.92 (s, 1H), 7.35 (s, 1H), 7.13 (d, J=8.3 Hz, 2H), 6.93 (d, J=7.5 Hz, 2H), 6.80 (s, 1H), 3.78 (s, 3H), 3.69 (s, 4H), 2.89 (s, 4H), 2.66-2.88 (m, 4H), 2.38-2.42 (m, 5H), 2.10 (s, 3H), 2.02 (d, J=14.4 Hz, 6H), 1.79 (d, J=13.0 Hz, 2H), 1.46 (s, 3H), 1.29 (s, 3H); [M+H]⁺=896.

### Example 95: 1-(4-(4-((4-(4-((5-bromo-4-((5-(dimethylphosphoryl)quinoxalin-6-yl)amino)pyrimidin-2-yl)amino)-5-methoxy-2-methylphenyl)piperazin-1-yl)methyl)piperidin-1-yl)phenyl)dihydropyrimidine-2,4(1H,3H)-dione

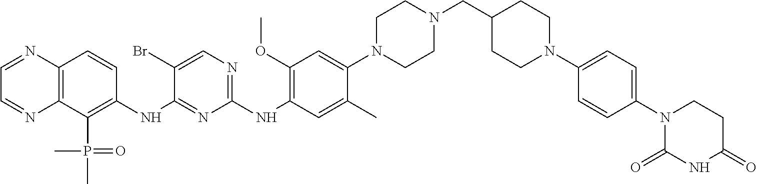 [SMILES: COc1cc(N2CCN(CC3CCN(c4ccc(N5CCC(=O)NC5=O)cc4)CC3)CC2)c(C)cc1Nc1ncc(Br)c(Nc2ccc3nccnc3c2P(C)(C)=O)n1]

The titled compound was synthesized in the procedures similar to Example 79. ¹H NMR (400 MHz, DMSO) δ 12.69 (s, 1H), 10.26 (s, 1H), 8.86 (d, J=5.7 Hz, 3H), 8.27 (s, 2H), 7.95 (s, 1H), 7.36 (s, 1H), 7.14 (d, J=8.5 Hz, 2H), 6.94 (d, J=7.6 Hz, 2H), 6.81 (s, 1H), 3.79 (s, 3H), 3.70 (s, 4H), 2.90 (s, 4H), 2.68 (s, 5H), 2.54 (s, 3H), 2.27 (s, 2H), 2.10 (s, 3H), 2.02 (d, J=14.4 Hz, 6H), 1.83 (m, 2H), 1.73 (s, 1H), 1.24 (m, 3H); [M+H]⁺=882.

### Example 96: 1-(4-(4-((4-(4-((5-bromo-4-((5-(dimethylphosphoryl)quinoxalin-6-yl)amino)pyrimidin-2-yl)amino)-5-methoxy-2-methylphenyl)piperazin-1-yl)methyl)piperidin-1-yl)-2-fluorophenyl)dihydropyrimidine-2,4(1H,3H)-dione

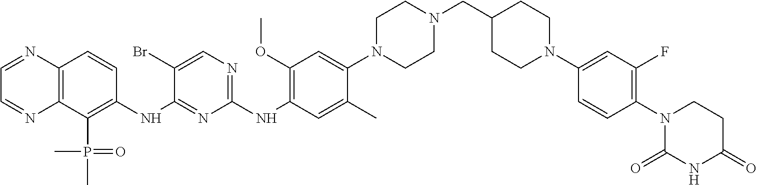 [SMILES: COc1cc(N2CCN(CC3CCN(c4ccc(N5CCC(=O)NC5=O)c(F)c4)CC3)CC2)c(C)cc1Nc1ncc(Br)c(Nc2ccc3nccnc3c2P(C)(C)=O)n1]

The titled compound was synthesized in the procedures similar to Example 82. ¹H NMR (400 MHz, DMSO) δ 12.67 (s, 1H), 10.36 (s, 1H), 8.84 (s, 3H), 8.25 (s, 2H), 7.92 (s, 1H), 7.32 (s, 1H), 7.17 (s, 1H), 6.78 (s, 3H), 3.76 (s, 4H), 3.60 (s, 1H), 2.87 (s, 4H), 2.67 (s, 4H), 2.23 (s, 4H), 2.11-1.91 (m, 10H), 1.77 (s, 5H), 1.20 (s, 3H); [M+H]⁺=900.

### Example 97: 1-(4-(4-((4-(4-((5-bromo-4-((5-(dimethylphosphoryl)quinoxalin-6-yl)amino)pyrimidin-2-yl)amino)-5-methoxy-2-methylphenyl)piperazin-1-yl)methyl)piperidin-1-yl)-3-fluorophenyl)dihydropyrimidine-2,4(1H,3H)-dione

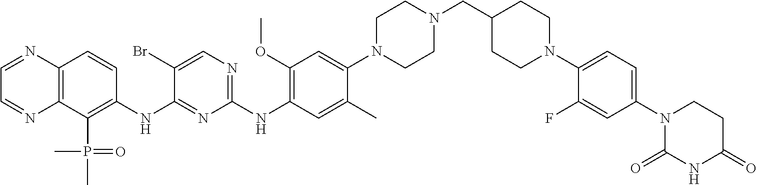 [SMILES: COc1cc(N2CCN(CC3CCN(c4ccc(N5CCC(=O)NC5=O)cc4F)CC3)CC2)c(C)cc1Nc1ncc(Br)c(Nc2ccc3nccnc3c2P(C)(C)=O)n1]

The titled compound was synthesized in the procedures similar to Example 82. ¹H NMR (400 MHz, DMSO) δ 12.69 (s, 1H), 10.35 (s, 1H), 8.86 (d, J=5.4 Hz, 3H), 8.27 (s, 2H), 7.94 (d, J=10.0 Hz, 1H), 7.36 (s, 1H), 7.17 (d, J=12.7 Hz, 1H), 7.06 (s, 2H), 6.81 (s, 1H), 3.79 (s, 3H), 3.74 (s, 2H), 3.34 (s, 2H), 2.90 (s, 4H), 2.68 (d, J=5.4 Hz, 4H), 2.52 (s, 4H), 2.28 (s, 2H), 2.10 (s, 3H), 2.02 (d, J=14.4 Hz, 6H), 1.85 (d, J=11.0 Hz, 2H), 1.71 (s, 1H), 1.32 (d, J=10.7 Hz, 2H); [M+H]⁺=900.

### Example 98: 1-(4-(4-((4-(4-((5-bromo-4-((5-(dimethylphosphoryl)quinoxalin-6-yl)amino)pyrimidin-2-yl)amino)-5-methoxy-2-methylphenyl)piperazin-1-yl)methyl)piperidin-1-yl)-3-methylphenyl)dihydropyrimidine-2,4(1H,3H)-dione

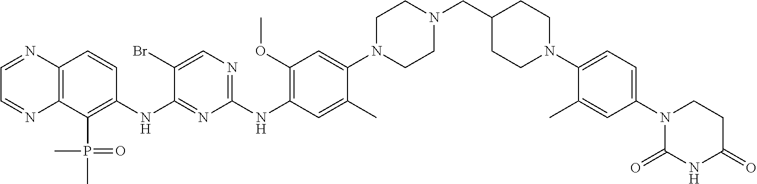 [SMILES: COc1cc(N2CCN(CC3CCN(c4ccc(N5CCC(=O)NC5=O)cc4C)CC3)CC2)c(C)cc1Nc1ncc(Br)c(Nc2ccc3nccnc3c2P(C)(C)=O)n1]

The titled compound was synthesized in the procedures similar to Example 84. ¹H NMR (400 MHz, DMSO) δ 12.69 (s, 1H), 10.28 (s, 1H), 8.86 (d, J=5.8 Hz, 3H), 8.27 (s, 2H), 7.94 (d, J=9.0 Hz, 1H), 7.36 (s, 1H), 7.11 (s, 2H), 7.02 (d, J=8.5 Hz, 1H), 6.80 (s, 1H), 3.79 (s, 3H), 3.71 (s, 2H), 3.06 (d, J=10.8 Hz, 2H), 2.90 (s, 4H), 2.73-2.55 (m, 7H), 2.29 (s, 2H), 2.24 (s, 3H), 2.10 (s, 3H), 2.02 (d, J=14.4 Hz, 6H), 1.86 (d, J=12.0 Hz, 2H), 1.70 (s, 1H), 1.31 (d, J=11.1 Hz, 2H); [M+H]⁺=896.

### Example 99: 1-(4-(4-((4-(4-((5-bromo-4-((5-(dimethylphosphoryl)quinoxalin-6-yl)amino)pyrimidin-2-yl)amino)-5-methoxy-2-methylphenyl)piperazin-1-yl)methyl)piperidin-1-yl)-2-methylphenyl)dihydropyrimidine-2,4(1H,3H)-dione

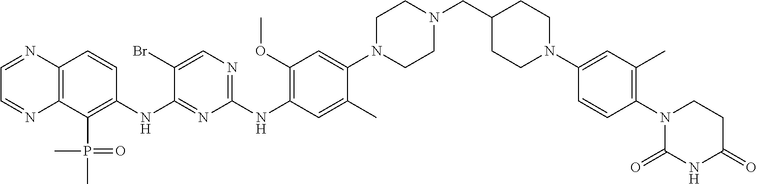 [SMILES: COc1cc(N2CCN(CC3CCN(c4ccc(N5CCC(=O)NC5=O)c(C)c4)CC3)CC2)c(C)cc1Nc1ncc(Br)c(Nc2ccc3nccnc3c2P(C)(C)=O)n1]

The titled compound was synthesized in the procedures similar to Example 82. ¹H NMR (400 MHz, DMSO) δ 12.78 (s, 1H), 10.34 (s, 1H), 8.95 (s, 3H), 8.35 (s, 2H), 8.01 (s, 1H), 7.44 (s, 1H), 7.12 (s, 1H), 6.90 (s, 3H), 3.87 (s, 2H), 3.77 (s, 3H), 2.98 (s, 4H), 2.60-2.80 (m, 7H), 2.34 (s, 2H), 2.25-2.05 (m, 12H), 1.70-1.95 (m, 5H), 1.32 (s, 3H); [M+H]⁺=896.

### Example 100: 5-(3-(4-(4-((5-bromo-4-((5-(dimethylphosphoryl)quinoxalin-6-yl)amino)pyrimidin-2-yl)amino)-5-methoxy-2-methylphenyl)piperazin-1-yl)azetidine-1-carbonyl)-N-(4-(2,6-dioxopiperidin-3-yl)phenyl)pyrazine-2-carboxamide

### Step 1: tert-butyl 3-(4-(5-methoxy-2-methyl-4-nitrophenyl)piperazin-1-yl)azetidine-1-carboxylate

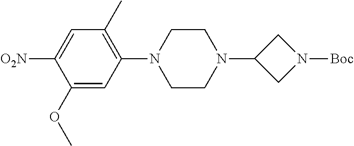 [SMILES: COc1cc(N2CCN(C3CN(C(=O)OC(C)(C)C)C3)CC2)c(C)cc1[N+](=O)[O-]]

A mixture of 1-fluoro-5-methoxy-2-methyl-4-nitrobenzene (500 mg, 2.70 mmol), tert-butyl 3-(piperazin-1-yl)azetidine-1-carboxylate (782 mg, 3.24 mmol) and K₂CO₃ (932 mg, 6.75 mmol) in DMF (10 mL) was stirred in a flask at 80° C. overnight. The reaction was cooled to room temperature, quenched with water and extracted with DCM (2×40 mL). The combined organic layers were washed with brine, dried over anhydrous Na₂SO₄, and evaporated in vacuum to afford the crude product, which was further purified with silica gel column chromatography (DCM: MeOH=100: 0˜20: 1 gradient elution) to give the product (350 mg, 32%). [M+H]⁺=407.3.

### Step 2: tert-butyl 3-(4-(4-amino-5-methoxy-2-methylphenyl)piperazin-1-yl)azetidine-1-carboxylate

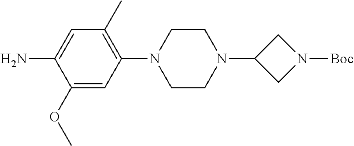 [SMILES: COc1cc(N2CCN(C3CN(C(=O)OC(C)(C)C)C3)CC2)c(C)cc1N]

Under N₂, to a solution of tert-butyl 3-(4-(5-methoxy-2-methyl-4-nitrophenyl)piperazin-1-yl)azetidine-1-carboxylate (350 mg, 0.861 mmol) in MeOH (15 mL) was added 10% Pd/C (100 mg) at 25° C. And then the mixture was exchanged with H₂ two times and stirred under H₂ atmosphere at 25° C. for 12 h. Reaction was monitored by LCMS. The mixture was filtered through a pad of Celite and washed with MeOH (15 mL). The filtrate was concentrated under vacuum to obtain the product (300 mg, 93%). [M+H]⁺*=377.4.

### Step 3: (6-((2-((4-(4-(azetidin-3-yl)piperazin-1-yl)-2-methoxy-5-methylphenyl)amino)-5-bromopyrimidin-4-yl)amino)quinoxalin-5-yl)dimethylphosphine oxide methanesulfonate

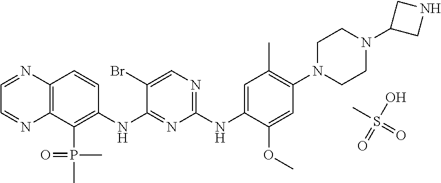

A mixture of (6-((5-bromo-2-chloropyrimidin-4-yl)amino)quinoxalin-5-yl)dimethylphosphine oxide (100 mg, 0.242 mmol), tert-butyl 3-(4-(4-amino-5-methoxy-2-methylphenyl)piperazin-1-yl)azetidine-1-carboxylate (119 mg, 0.315 mmol) and MsOH (93 mg, 0.968 mmol) in t-BuOH (10 mL) was stirred in a flask at 100° C. overnight under N₂. The mixture was evaporated in vacuum. The residue was diluted with DCM (10 mL) and washed with H₂O (2×20 mL) and brine(2×10 mL), dried over anhydrous Na₂SO₄, and evaporated in vacuum to afford the crude product which was used for next step without further purification (110 mg, 61%). [M+H]⁺=652.5.

### Step 4: 5-(3-(4-(4-((5-bromo-4-((5-(dimethylphosphoryl)quinoxalin-6-yl)amino)pyrimidin-2-yl)amino)-5-methoxy-2-methylphenyl)piperazin-1-yl)azetidine-1-carbonyl)-N-(4-(2,6-dioxopiperidin-3-yl)phenyl)pyrazine-2-carboxamide

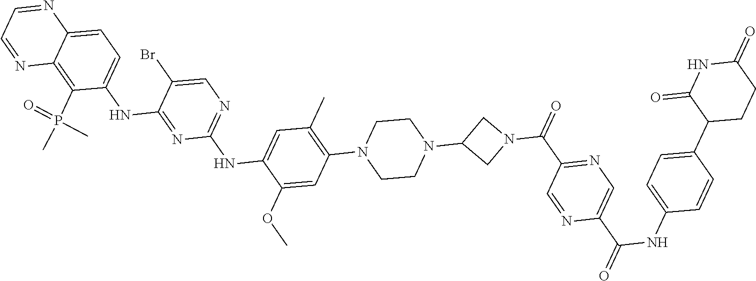 [SMILES: COc1cc(N2CCN(C3CN(C(=O)c4cnc(C(=O)Nc5ccc(C6CCC(=O)NC6=O)cc5)cn4)C3)CC2)c(C)cc1Nc1ncc(Br)c(Nc2ccc3nccnc3c2P(C)(C)=O)n1]

To a solution of 5-((4-(2,6-dioxopiperidin-3-yl)phenyl)carbamoyl)pyrazine-2-carboxylic acid (30.6 mg, 0.086 mmol), HATU (36 mg, 0.095 mmol) and DIEA (33 mg, 0.258 mmol) in DMF (3 mL) was added (6-((2-((4-(4-(azetidin-3-yl)piperazin-1-yl)-2-methoxy-5-methylphenyl)amino)-5-bromopyrimidin-4-yl)amino)quinoxalin-5-yl)dimethylphosphine oxide methanesulfonate (64 mg, 0.086 mmol). The resulting mixture was stirred at room temperature for 5 h. The reaction was directly purified with pre-HPLC to give the title product (6 mg, 7%). ¹H NMR (400 MHz, DMSO) δ 12.70 (s, 1H), 10.93 (s, 1H), 10.86 (s, 1H), 9.26 (d, J=36.9 Hz, 1H), 8.87 (d, J=6.7 Hz, 3H), 8.29 (d, J=15.8 Hz, 2H), 7.93 (d, J=8.4 Hz, 1H), 7.86 (d, J=7.2 Hz, 2H), 7.36 (s, 1H), 7.24 (d, J=7.0 Hz, 2H), 6.80 (s, 1H), 4.69 (s, 1H), 4.49 (s, 1H), 4.23 (s, 1H), 4.04 (s, 1H), 3.85 (d, J=8.0 Hz, 1H), 3.79 (s, 3H), 2.93 (s, 5H), 2.69 (s, 2H), 2.53 (s, 3H), 2.23 (d, J=11.6 Hz, 1H), 2.11 (s, 3H), 2.03 (d, J=14.4 Hz, 8H).[M+H]⁺=988.5.

### Example 101: 3-(4-(2-(4-(4-((5-bromo-4-((5-(dimethylphosphoryl)quinoxalin-6-yl)amino)pyrimidin-2-yl)amino)-3-methoxyphenethyl)piperidin-1-yl)ethyl)phenyl)piperidine-2,6-dione

### Step 1: Tert-butyl(E)-4-(3-methoxy-4-nitrostyryl)piperidine-1-carboxylate

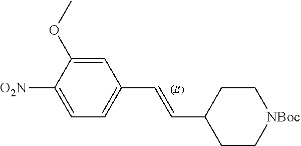 [SMILES: COc1cc(/C=C/C2CCN(C(=O)OC(C)(C)C)CC2)ccc1[N+](=O)[O-]]

A mixture of 4-bromo-2-methoxy-1-nitrobenzene (500 mg, 2.16 mmol), tert-butly 4-vinylpiperidine-1-carboxylate (455 mg, 2.16 mmol), TEA (1.09 g, 10.79 mmol) and dichlorobis(tri-O— tolylphosphine)palladium(II) (C:40691-33-6) (170 mg, 0.216 mmol) in DMF (5 mL) was stirred at 130° C. for 1.5 h under N₂. The reaction was quenched with water and the mixture was washed with saturated brine, then extracted with EtOAc. The organic layer was dried over anhydrous Na₂SO₄, and evaporated in vacuum to afford the crude product, which was further purified by Prep-TLC with (EA:PE=1:2) to give the product (180 mg, 23.0%). [M+H]⁺=363.

Step 2: Tert-butyl-4-(4-amino-3-methoxyphenethyl)piperidine-1-carboxylate

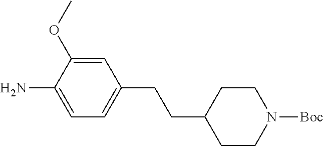 [SMILES: COc1cc(CCC2CCN(C(=O)OC(C)(C)C)CC2)ccc1N]

A mixture of tert-butyl(E)-4-(3-methoxy-4-nitrostyryl)piperidine-1-carboxylate (180 mg, 0.50 mmol), 10% Pd/C (180 mg), AcOH (0.02 m1) in MeOH (3 m1) was stirred overnight under H₂ at RT. The solid was filtered out and the filtrate was concentrated under reduced pressure to give the product (160 mg, 96.4%). [M+H]⁺=335.

### Step 3: (6-((5-bromo-2-((2-methoxy-4-(2-(piperidin-4-yl)ethyl)phenyl)amino)pyrimidin-4-yl)amino)quinoxalin-5-yl)dimethylphosphine oxide

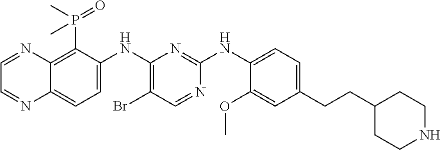 [SMILES: COc1cc(CCC2CCNCC2)ccc1Nc1ncc(Br)c(Nc2ccc3nccnc3c2P(C)(C)=O)n1]

The titled compound was prepared in a manner similar to that in Example 23 step 6. [M+H]⁺=610.

### Step 4: 3-(4-(2-(4-(4-((5-bromo-4-((5-(dimethylphosphoryl)quinoxalin-6-yl)amino)pyrimidin-2-yl)amino)-3-methoxyphenethyl)piperidin-1-yl)ethyl)phenyl)piperidine-2,6-dione

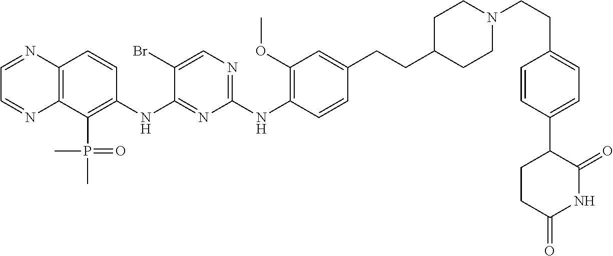 [SMILES: COc1cc(CCC2CCN(CCc3ccc(C4CCC(=O)NC4=O)cc3)CC2)ccc1Nc1ncc(Br)c(Nc2ccc3nccnc3c2P(C)(C)=O)n1]

The titled compound was synthesized in a manner similar to that in Example 79 step 8 from (2-((5-chloro-2-((2-methoxy-4-(2-(piperidin-4-yl)ethyl)phenyl)amino)pyrimidin-4-yl)amino)phenyl)dimethylphosphine oxide and 2-(4-(2,6-dioxopiperidin-3-yl)phenyl) acetaldehyde. ¹H NMR (400 MHz, DMSO) δ 10.82 (s, 1H), 8.86 (d, J=9.5 Hz, 3H), 8.45-8.25 (m, 2H), 7.98 (s, 1H), 7.52 (s, 1H), 7.14 (d, J=15.2 Hz, 4H), 6.94 (s, 1H), 6.74 (s, 1H), 3.80 (s, 4H), 2.85-3.00 (m, 4H), 2.58-2.70 (m, 6H), 2.16 (s, 1H), 2.02 (d, J=14.3 Hz, 6H), 1.87 (s, 2H), 1.72 (s, 2H), 1.54 (s, 2H), 1.24 (m, 4H); [M+H]⁺=825.

### Example 102: (2S,4R)-1-((S)-2-(4-(4-(4-((5-bromo-4-((5-(dimethylphosphoryl)quinoxalin-6-yl)amino)pyrimidin-2-yl)amino)-5-methoxy-2-methylphenyl)piperazin-1-yl)butanamido)-3,3-dimethylbutanoyl)-4-hydroxy-N—((S)-1-(4-(4-methylthiazol-5-yl)phenyl)ethyl)pyrrolidine-2-carboxamide

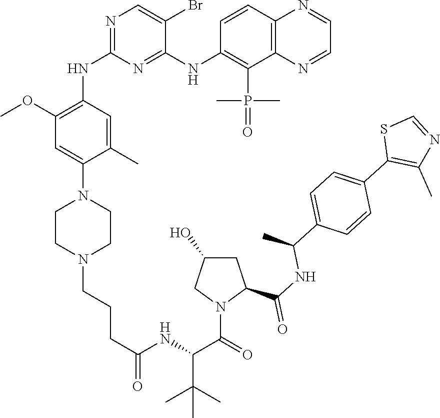 [SMILES: COc1cc(N2CCN(CCCC(=O)N[C@H](C(=O)N3C[C@H](O)C[C@H]3C(=O)N[C@@H](C)c3ccc(-c4scnc4C)cc3)C(C)(C)C)CC2)c(C)cc1Nc1ncc(Br)c(Nc2ccc3nccnc3c2P(C)(C)=O)n1]

The titled compound was synthesized in the procedures similar to Example 103. ¹H NMR (400 MHz, DMSO) δ 12.70 (s, 1H), 8.99 (s, 1H), 8.86 (s, 3H), 8.40 (s, 1H), 8.27 (s, 2H), 7.93 (s, 2H), 7.49-7.27 (m, 5H), 6.78 (s, 1H), 5.12 (s, 1H), 4.92 (s, 1H), 4.53 (s, 1H), 4.43 (s, 1H), 4.29 (s, 1H), 3.79 (s, 3H), 3.62 (s, 2H), 2.92 (s, 5H), 2.62 (d, J=44.6 Hz, 4H), 2.45 (s, 6H), 2.13-1.88 (m, 10H), 1.74 (s, 3H), 1.38 (s, 3H), 0.96 (s, 9H); [M+H]⁺=1109.

### Example 103: (2S,4R)-1-((S)-2-(3-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethoxy)propanamido)-3,3-dimethylbutanoyl)-4-hydroxy-N-(4-(4-methylthiazol-5-yl)benzyl)pyrrolidine-2-carboxamide

### Step 1: tert-butyl 3-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethoxy)propanoate

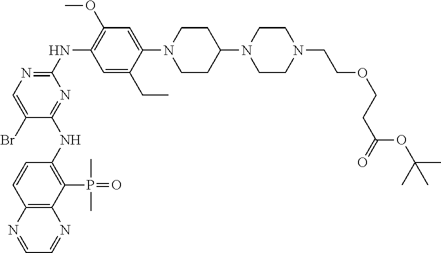 [SMILES: CCc1cc(Nc2ncc(Br)c(Nc3ccc4nccnc4c3P(C)(C)=O)n2)c(OC)cc1N1CCC(N2CCN(CCOCCC(=O)OC(C)(C)C)CC2)CC1]

To a solution of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (90 mg, 0.13 mmol) and tert-butyl 3-(2-bromoethoxy)propanoate (50 mg, 0.20 mmol) in DMF(2 mL) was added K₂CO₃(37 mg, 0.26 mmol). The resulting mixture was heated at 90° C. for 1 hour. The crude solution was purified with flash C18 column chromatography to give the title product (50 mg, 45%). [M+H]⁺=866.

### Step 2: 3-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-1)ethoxy)propanoic acid

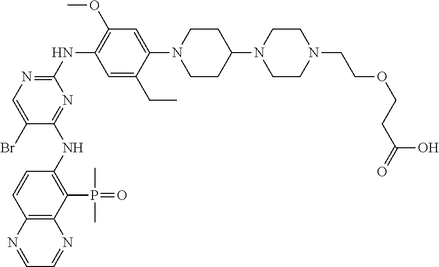 [SMILES: CCc1cc(Nc2ncc(Br)c(Nc3ccc4nccnc4c3P(C)(C)=O)n2)c(OC)cc1N1CCC(N2CCN(CCOCCC(=O)O)CC2)CC1]

A solution of tert-butyl 3-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethoxy)propanoate (50 mg, 0.057 mmol) in HCl/1,4-dioxane (3 mL) was stirred in a round bottom flask at room temperature overnight. The mixture was evaporated in vacuum to afford the crude product (40 mg, 87%), which was used for next step without further purification. [M+H]⁺=810.

### Step 3: (2S,4R)-1-((S)-2-(3-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethoxy)propanamido)-3,3-dimethylbutanoyl)-4-hydroxy-N-(4-(4-methylthiazol-5-yl)benzyl)pyrrolidine-2-carboxamide

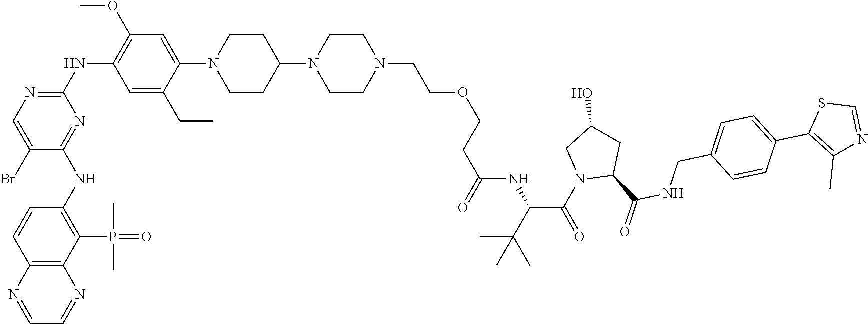 [SMILES: CCc1cc(Nc2ncc(Br)c(Nc3ccc4nccnc4c3P(C)(C)=O)n2)c(OC)cc1N1CCC(N2CCN(CCOCCC(=O)N[C@H](C(=O)N3C[C@H](O)C[C@H]3C(=O)NCc3ccc(-c4scnc4C)cc3)C(C)(C)C)CC2)CC1]

To a solution of 3-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethoxy)propanoic acid (40 mg, 0.049 mmol), HATU (22 mg, 0.05 mmol) and DIEA (24 mg, 0.15 mmol) in DMF(2 mL) was added (2S,4R)-1-((S)-2-amino-3,3-dimethylbutanoyl)-4-hydroxy-N-(4-(4-methylthiazol-5-yl)benzyl)pyrrolidine-2-carboxamide (26 mg, 0.05 mmol). The resulting mixture was stirred at room temperature for 1 h. The reaction was directly purified with pre-HPLC to give the title product (10 mg, 16.6%). ¹H NMR (400 MHz, DMSO) δ 12.65 (s, 1H), 8.98 (s, 1H), 8.86 (d, J=7.7 Hz, 3H), 8.59 (s, 1H), 8.29 (d, J=10.1 Hz, 2H), 7.97 (s, 1H), 7.90 (d, J=9.6 Hz, 1H), 7.40 (d, J=9.5 Hz, 5H), 6.81 (s, 1H), 5.16 (s, 1H), 4.57 (d, J=9.2 Hz, 1H), 4.43 (s, 2H), 4.36 (s, 1H), 4.22 (d, J=10.9 Hz, 1H), 3.77 (s, 3H), 3.64 (m, 7H), 3.02 (m, 4H), 2.71 (m, 4H), 2.50-2.54 (m, 5H), 2.40-2.48 (m, 7H), 2.02 (d, J=14.4 Hz, 8H), 1.91 (s, 3H), 1.63 (s, 2H), 0.95 (m, 13H); [M+H]⁺=1222.

### Example 104: 3-(4-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)ethyl)phenyl)piperidine-2,6-dione

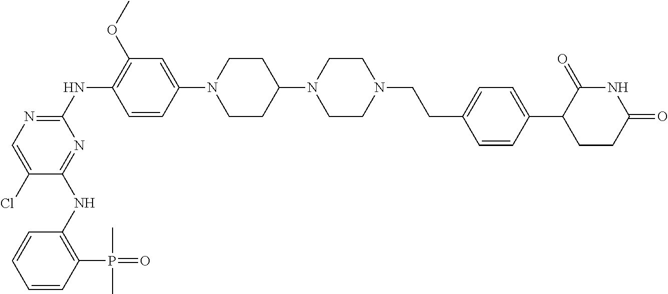 [SMILES: COc1cc(N2CCC(N3CCN(CCc4ccc(C5CCC(=O)NC5=O)cc4)CC3)CC2)ccc1Nc1ncc(Cl)c(Nc2ccccc2P(C)(C)=O)n1]

The titled compound was synthesized in the procedures similar to Example 23. ¹H NMR (400 MHz, DMSO) δ_{H} 11.18 (s, 1H), 10.83 (s, 1H), 8.48 (s, 1H), 8.07 (d, J=5.9 Hz, 2H), 7.53 (dd, J=14.1, 7.5 Hz, 1H), 7.42-7.29 (m, 2H), 7.13 (dt, J=13.5, 7.5 Hz, 5H), 6.63 (s, 1H), 6.47 (d, J=8.5 Hz, 1H), 3.84-3.68 (m, 6H), 3.09-2.89 (m, 1H), 2.75-2.60 (m, 6H), 2.54 (s, 5H), 2.45-2.30 (m, 5H), 2.17 (d, J=8.5 Hz, 1H), 2.03 (s, 1H), 1.86 (d, J=11.9 Hz, 2H), 1.76 (d, J=13.5 Hz, 6H), 1.53 (d, J=10.4 Hz, 2H); [M+H]⁺=785.4.

### Example 105: 3-(4-(4-((4-(4-((5-chloro-4-((2-(dimethylphosphoryl)phenyl)amino)pyrimidin-2-yl)amino)-3-methoxyphenyl)piperazin-1-yl)methyl)piperidin-1-yl)phenyl)piperidine-2,6-dione

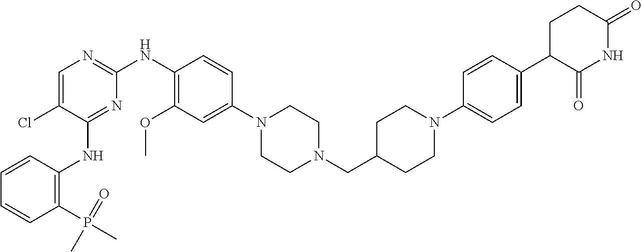 [SMILES: COc1cc(N2CCN(CC3CCN(c4ccc(C5CCC(=O)NC5=O)cc4)CC3)CC2)ccc1Nc1ncc(Cl)c(Nc2ccccc2P(C)(C)=O)n1]

The titled compound was synthesized in the procedures similar to Example 23. ¹H NMR (400 MHz, DMSO) δ 11.19 (s, 1H), 10.79 (s, 1H), 8.49 (s, 1H), 8.08 (d, J=10.7 Hz, 2H), 7.54 (s, 1H), 7.46-7.29 (m, 2H), 7.11 (s, 1H), 7.04 (d, J=8.0 Hz, 2H), 6.89 (d, J=7.8 Hz, 2H), 6.64 (s, 1H), 6.47 (d, J=8.2 Hz, 1H), 3.77 (s, 3H), 3.68 (d, J=14.3 Hz, 3H), 3.15 (s, 4H), 2.72-2.52 (m, 8H), 2.24 (d, J=6.9 Hz, 2H), 2.12 (s, 1H), 2.01 (s, 1H), 1.79 (t, J=18.0 Hz, 9H), 1.23 (d, J=10.9 Hz, 2H); [M+H]⁺=771.3.

### Example 106: 3-(4-(4-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)ethyl)piperidin-1-yl)phenyl)piperidine-2,6-dione

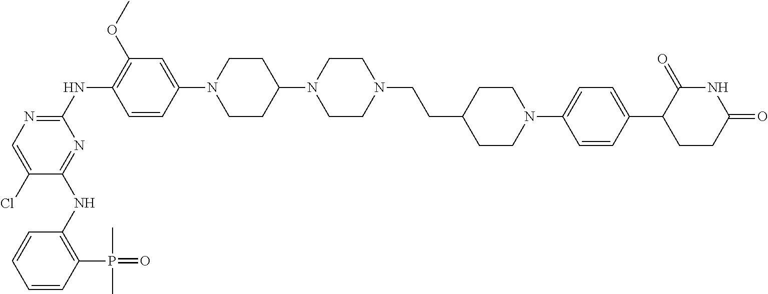 [SMILES: COc1cc(N2CCC(N3CCN(CCC4CCN(c5ccc(C6CCC(=O)NC6=O)cc5)CC4)CC3)CC2)ccc1Nc1ncc(Cl)c(Nc2ccccc2P(C)(C)=O)n1]

The titled compound was synthesized in the procedures similar to Example 23. ¹H NMR (400 MHz, DMSO) δ_{H} 11.19 (s, 1H), 10.78 (s, 1H), 8.48 (s, 1H), 8.07 (d, J=5.1 Hz, 2H), 7.58-7.47 (m, 1H), 7.42-7.28 (m, 2H), 7.10 (d, J=7.1 Hz, 1H), 7.03 (d, J=8.2 Hz, 2H), 6.88 (d, J=8.4 Hz, 2H), 6.63 (s, 1H), 6.47 (d, J=8.6 Hz, 1H), 3.74 (d, J=15.4 Hz, 6H), 3.64 (d, J=11.5 Hz, 2H), 3.12-2.88 (m, 1H), 2.72-2.52 (m, 10H), 2.43 (s, 6H), 2.11 (s, 1H), 2.01 (s, 1H), 1.88 (d, J=11.3 Hz, 2H), 1.76 (d, J=13.3 Hz, 8H), 1.54 (d, J=10.2 Hz, 2H), 1.42 (s, 3H), 1.24 (d, J=7.5 Hz, 2H); [M+H]⁺=868.4.

### Example 107: 3-(4-(4-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-2-oxoethyl)piperidin-1-yl)phenyl)piperidine-2,6-dione

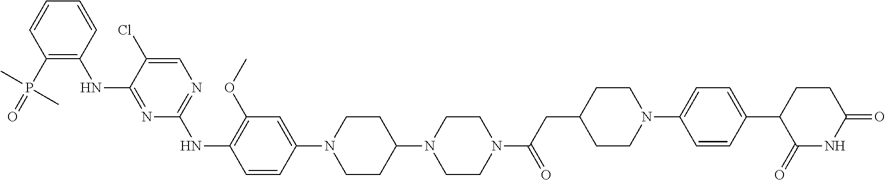 [SMILES: COc1cc(N2CCC(N3CCN(C(=O)CC4CCN(c5ccc(C6CCC(=O)NC6=O)cc5)CC4)CC3)CC2)ccc1Nc1ncc(Cl)c(Nc2ccccc2P(C)(C)=O)n1]

The titled compound was synthesized in the procedures similar to Example 24. ¹H NMR (400 MHz, DMSO) δ_{H} 11.19 (s, 1H), 10.78 (s, 1H), 8.48 (s, 1H), 8.08 (d, J=6.3 Hz, 2H), 7.53 (dd, J=13.8, 7.7 Hz, 1H), 7.43-7.29 (m, 2H), 7.10 (d, J=7.3 Hz, 1H), 7.03 (d, J=8.1 Hz, 2H), 6.88 (d, J=8.2 Hz, 2H), 6.64 (s, 1H), 6.48 (d, J=8.1 Hz, 1H), 3.83-3.60 (m, 9H), 3.47 (s, 3H), 2.70-2.61 (m, 7H), 2.43 (s, 2H), 2.28 (s, 3H), 2.12 (d, J=9.4 Hz, 1H), 2.01 (s, 1H), 1.88 (s, 3H), 1.76 (d, J=13.5 Hz, 9H), 1.59 (s, 2H), 1.34-1.22 (m, 2H); [M+H]⁺=882.4.

### Example 108: 3-((4-(2-(4-(1-(4-((5-chloro-4-((2-(dimethylphosphoryl)phenyl)amino)pyrimidin-2-yl)amino)-3-methoxyphenyl piperidin-4-yl)piperazin-1-yl)ethyl)phenyl)amino)piperidine-2,6-dione

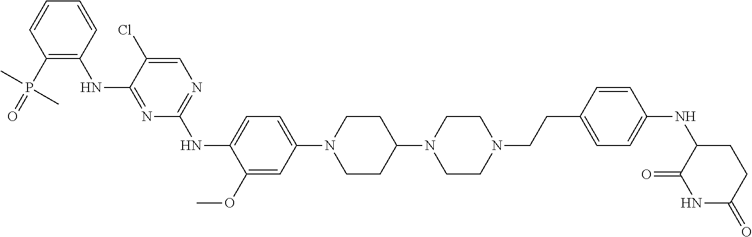 [SMILES: COc1cc(N2CCC(N3CCN(CCc4ccc(NC5CCC(=O)NC5=O)cc4)CC3)CC2)ccc1Nc1ncc(Cl)c(Nc2ccccc2P(C)(C)=O)n1]

The titled compound was synthesized in the procedures similar to Example 48. ¹H NMR (400 MHz, DMSO) δ_{H} 11.18 (s, 1H), 10.78 (s, 1H), 8.48 (s, 1H), 8.07 (s, 2H), 7.53 (dd, J=14.2, 7.5 Hz, 1H), 7.36 (s, 2H), 7.09 (s, 1H), 6.93 (d, J=7.9 Hz, 2H), 6.66-6.55 (m, 3H), 6.47 (d, J=8.1 Hz, 1H), 5.64 (s, 1H), 4.26 (s, 1H), 3.73 (d, J=18.8 Hz, 5H), 3.04-2.94 (m, 1H), 2.68 (d, J=11.5 Hz, 4H), 2.56 (d, J=16.7 Hz, 5H), 2.42 (d, J=6.4 Hz, 5H), 2.33 (s, 2H), 2.09 (s, 1H), 1.86 (d, J=10.8 Hz, 3H), 1.76 (d, J=13.4 Hz, 6H), 1.54 (s, 2H); [M+H]⁺=800.4.

### Example 109: 3-(4-(3-(4-(1-(4-((5-chloro-4-((2-(dimethylphosphoryl)phenyl)amino)pyrimidin-2-yl)amino)-3-methoxyphenyl)piperidin-4-yl)piperazin-1-yl)propoxy)phenoxy)piperidine-2,6-dione

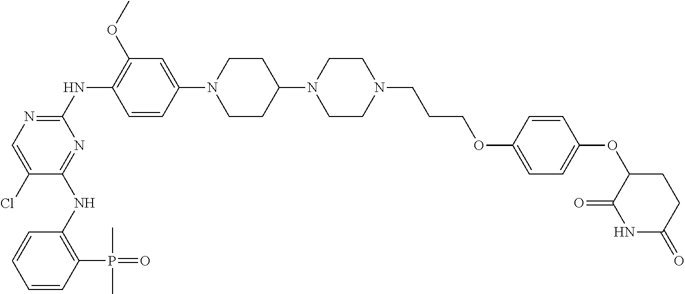 [SMILES: COc1cc(N2CCC(N3CCN(CCCOc4ccc(OC5CCC(=O)NC5=O)cc4)CC3)CC2)ccc1Nc1ncc(Cl)c(Nc2ccccc2P(C)(C)=O)n1]

The titled compound was synthesized in the procedures similar to Example 92. ¹H NMR (400 MHz, DMSO) δ_{H} 11.18 (s, 1H), 10.91 (s, 1H), 8.48 (s, 1H), 8.07 (d, J=7.0 Hz, 2H), 7.53 (dd, J=13.9, 7.6 Hz, 1H), 7.42-7.28 (m, 2H), 7.09 (t, J=7.3 Hz, 1H), 6.94 (d, J=8.9 Hz, 2H), 6.85 (d, J=8.9 Hz, 2H), 6.63 (s, 1H), 6.47 (d, J=8.6 Hz, 1H), 5.07-4.99 (m, 1H), 3.94 (s, 2H), 3.73 (d, J=19.4 Hz, 5H), 2.71-2.60 (m, 4H), 2.52 (s, 4H), 2.45-2.32 (m, 7H), 2.22-2.05 (m, 2H), 1.84 (s, 4H), 1.76 (d, J=13.5 Hz, 6H), 1.53 (d, J=11.0 Hz, 2H); [M+H]⁺=831.4.

### Example 110: 1-(4-(4-((4-(1-(4-((5-chloro-4-((2-(dimethylphosphoryl)phenyl)amino)pyrimidin-2-yl)amino)-3-methoxyphenyl)piperidin-4-yl)piperazin-1-yl)methyl)cyclohexyl)phenyl)dihydropyrimidine-2,4(1H,3H)-dione

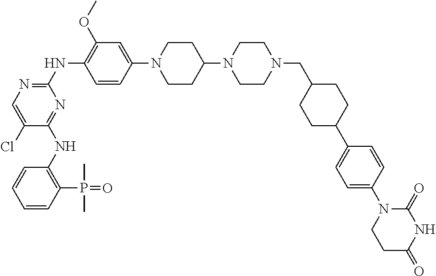 [SMILES: COc1cc(N2CCC(N3CCN(CC4CCC(c5ccc(N6CCC(=O)NC6=O)cc5)CC4)CC3)CC2)ccc1Nc1ncc(Cl)c(Nc2ccccc2P(C)(C)=O)n1]

The titled compound was synthesized in the procedures similar to Example 79. ¹H NMR (400 MHz, DMSO) δ_{H} 11.18 (s, 1H), 10.27 (s, 1H), 8.49 (s, 1H), 8.07 (s, 2H), 7.52 (d, J=13.2 Hz, 1H), 7.43-7.28 (m, 2H), 7.13 (d, J=8.2 Hz, 3H), 6.92 (d, J=7.6 Hz, 2H), 6.63 (s, 1H), 6.47 (d, J=8.1 Hz, 1H), 3.80-3.63 (m, 10H), 2.72-2.54 (m, 9H), 2.37 (s, 5H), 2.15 (s, 2H), 1.86 (d, J=10.5 Hz, 2H), 1.77 (d, J=13.1 Hz, 8H), 1.66 (s, 1H), 1.53 (d, J=11.4 Hz, 2H), 1.19 (d, J=10.7 Hz, 2H); [M+H]⁺=855.4.

### Example 111: 1-(4-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)ethyl)benzyl)dihydropyrimidine-2,4(1H,3H)-dione

### Step 1: 2-(4-(bromomethyl)phenyl)ethan-1-ol

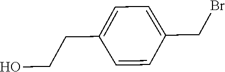 [SMILES: OCCc1ccc(CBr)cc1]

To a solution of 2-(4-(bromomethyl)phenyl)acetic acid (50.0 g, 218 mmol) in THF (250 mL) was added dropwise BH₃⁻THF (1M, 261 mL) at 0° C. The mixture was stirred at 15° C. for 5 hrs. TLC (Petroleum ether: Ethyl acetate=1: 1) showed the reaction was completed. The mixture was added sat. K₂CO₃ (200 mL), the THF of the biphasic mixture was removed in vacuo and the resulting aqueous phase was extracted with EtOAc (250 mL×2). The combined organic phases were washed with brine (100 mL), dried over Na₂SO₄, filtered and concentrated in vacuum to provide the product (46.0 g, 213 mmol, 97.9%). ¹H NMR (400 MHz, CDCl₃) δ_{H} 7.36 (d, J=8.16 Hz, 2H), 7.22 (d, J=8.16 Hz, 2H), 4.50 (s, 2H), 3.87 (q, J=6.18 Hz, 2H), 2.88 (t, J=6.62 Hz, 2H), 1.39 (t, J=5.96 Hz, 1H).

### Step 2: (4-(bromomethyl)phenethoxy)(tert-butyl)dimethylsilane

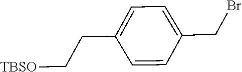 [SMILES: CC(C)(C)[Si](C)(C)OCCc1ccc(CBr)cc1]

To a solution of 2-(4-(bromomethyl)phenyl)ethan-1-ol (46.0 g, 213 mmol) in DCM (230 mL) was added imidazole (15.2 g, 224 mmol) and TBSCl (33.8 g, 224 mmol) at 0° C. The mixture was stirred at 15° C. for 2 hrs. The mixture was filtered and the filtrate was concentrated in vacuum to provide the product (68.0 g, 96.5%). [M+H]⁺=329.

### Step 3: 1-(4-(2-((tert-butyldimethylsilyl)oxy)ethyl)benzyl)pyrimidine-2.4(1H,3H)-dione

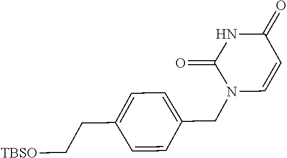 [SMILES: CC(C)(C)[Si](C)(C)OCCc1ccc(Cn2ccc(=O)[nH]c2=O)cc1]

To a solution of (4-(bromomethyl)phenethoxy)(tert-butyl)dimethylsilane (29.0 g, 88.0 mmol) in DMF (150 mL) was added Cs₂CO₃ (43.0 g, 132 mmol) and 1H-pyrimidine-2,4-dione (10.8 g, 96.8 mmol) at 15° C. The mixture was stirred at 15° C. for 12 hrs. The mixture was poured into water (1500 mL) and was added EtOAc (400 mL). Then the mixture was partitioned between EtOAc and water. And the aqueous phase was extracted with EtOAc (300 mL×2). The combined organic phases were washed with brine (300 mL), dried over Na₂SO₄, filtered and concentrated in vacuum. The residue was purified by column chromatography (SiO₂, Petroleum ether: Ethyl acetate=10: 1 to 1: 2) to afford the product (23.0 g, 72.5%). ¹H NMR (400 MHz, CDCl₃) δ_{H} 8.08 (s, 2H), 7.32 (s, 1H), 7.25-7.29 (m, 2H), 7.18 (d, J=7.96 Hz, 1H), 5.72 (dd, J=7.96, 2.20 Hz, 1H), 4.94 (s, 2H), 3.85 (t, J=6.92 Hz, 2H), 2.87 (t, J=6.86 Hz, 2H), 0.91 (s, 9H), 0.02 (s, 6H).

### Step 4: 1-(4-(2-((tert-butyldimethylsilyl)oxy)ethyl)benzyl)dihydropyrimidine-2,4(1H,3H)-dione

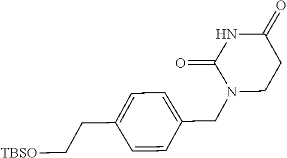 [SMILES: CC(C)(C)[Si](C)(C)OCCc1ccc(CN2CCC(=O)NC2=O)cc1]

To a solution of 1-(4-(2-((tert-butyldimethylsilyl)oxy)ethyl)benzyl)pyrimidine-2,4(1H,3H)-dione (16.0 g, 44.3 mmol) in MeOH (160 mL) was added Pd/C (2.00 g, 10.0% purity) under N₂ atmosphere. The suspension was degassed and purged with H₂ for 3 times. The mixture was stirred under H₂ (50.0 Psi) at 50° C. for 5 hrs. The mixture was filtered, the filtrate was concentrated under reduced pressure to provide the product (13.0 g, 80.8% yield). ¹H NMR (400 MHz, CDCl₃)⁶H 7.43 (br s, 1H), 7.33 (s, 2H), 4.65 (s, 2 H), 3.86 (t, J=6.84 Hz, 2H), 3.37 (t, J=6.84 Hz, 2H), 2.88 (t, J=6.84 Hz, 2H), 2.67 (t, J=6.74 Hz, 2H), 0.03 (s, 6H), 0.92 (s, 9H).

### Step 5: 1-(4-(2-hydroxyethyl)benzyl)dihydropyrimidine-2,4(1H,3H)-dione

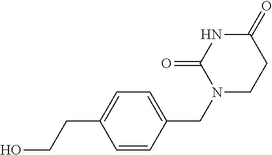 [SMILES: O=C1CCN(Cc2ccc(CCO)cc2)C(=O)N1]

To a solution of 1-(4-(2-((tert-butyldimethylsilyl)oxy)ethyl)benzyl)dihydropyrimidine-2,4(1H,3H)-dione (10.0 g, 27.5 mmol) in MeOH (50.0 mL) was added NH₄F (4.09 g, 110 mmol). The mixture was stirred at 25° C. for 8 hrs. Evaporate the solution on a water bath under reduced pressure using a rotary evaporator. Then was added H₂O (100 mL) and EtOAc (80 mL), the reaction mixture was partitioned between EtOAc and water. And the resulting aqueous phase was extracted with EtOAc (40 mL×2). The combined organic phase was washed with brine (40 mL), dried over Na₂SO₄, filtered and concentrated in vacuum. The crude product was triturated with EtOAc (50 mL) at 15° C. for 2 hrs to afford the product (4.84 g, 70.9% yield). ¹H NMR (400 MHz, CDCl₃) δ_{H} 7.46-7.52 (m, 1H), 7.27 (d, J=2.22 Hz, 1H), 7.25 (s, 3 H), 4.60 (d, J=1.76 Hz, 2H), 3.85-3.92 (m, 2H), 3.34 (td, J=6.78, 2.09 Hz, 2H), 2.86-2.92 (m, 2H), 2.64 (td, J=6.78, 1.87 Hz, 2H), 1.41 (td, J=5.73, 1.76 Hz, 1H); [M+H]⁺=249.

### Step 6: 2-(4-((2,4-dioxotetrahydropyrimidin-1(2H)-yl)methyl)phenyl)acetaldehyde

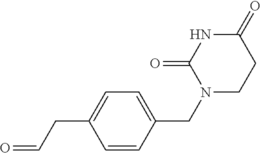 [SMILES: O=CCc1ccc(CN2CCC(=O)NC2=O)cc1]

The titled compound was synthesized in a manner similar to that in Example 79 step 7 from 1-(4-(2-hydroxyethyl)benzyl)dihydropyrimidine-2,4(1H,3H)-dione and IBX. [M+H]⁺=247.1.

### Step 7: 1-(4-(2-(4-(1-(4-((5-chloro-4-((2-(dimethylphosphoryl)phenyl)amino)pyrimidin-2-yl)amino)-3-methoxyphenyl)piperidin-4-yl)piperazin-1-yl)ethyl)benzyl)dihydropyrimidine-2,4(1H,3H)-dione

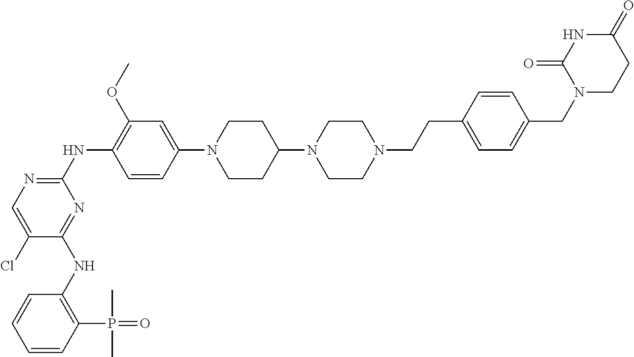 [SMILES: COc1cc(N2CCC(N3CCN(CCc4ccc(CN5CCC(=O)NC5=O)cc4)CC3)CC2)ccc1Nc1ncc(Cl)c(Nc2ccccc2P(C)(C)=O)n1]

The titled compound was synthesized in a manner similar to that in Example 79 step 8 from (2-((5-chloro-2-((2-methoxy-4-(4-(piperazin-1-yl)piperidin-1-yl)phenyl)amino)pyrimidin-4-yl)amino)phenyl)dimethylphosphine oxide (The synthesis of this intermediate is described in WO 2019196812) and 2-(4-((2,4-dioxotetrahydropyrimidin-1(2H)-yl)methyl)phenyl)acetaldehyde. ¹H NMR (400 MHz, DMSO) δ_{H} 11.18 (s, 1H), 10.20 (s, 1H), 8.48 (s, 1H), 8.07 (d, J=7.5 Hz, 2H), 7.53 (dd, J=14.3, 7.8 Hz, 1H), 7.37 (d, J=8.8 Hz, 2H), 7.19 (s, 4H), 7.09 (s, 1H), 6.63 (s, 1H), 6.47 (d, J=8.4 Hz, 1H), 4.47 (s, 2H), 3.73 (d, J=18.3 Hz, 5H), 3.26 (t, J=6.5 Hz, 2H), 2.74-2.62 (m, 4H), 2.54 (s, 5H), 2.48-2.30 (m, 8H), 1.86 (d, J=11.5 Hz, 2H), 1.76 (d, J=13.6 Hz, 6H), 1.52 (d, J=11.5 Hz, 2H); [M+H]⁺=800.4.

### Example 112: 5-(4-(3-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-3-oxopropyl)piperazin-1-yl)-2-(2,6-dioxopiperidin-3-yl)isoindoline-1,3-dione

To a solution of 3-(4-(2-(2,6-dioxopiperidin-3-yl)-1,3-dioxoisoindolin-5-yl)piperazin-1-yl)propanoic acid (17 mg, 0.04 mmol), HATU (15 mg, 0.04 mmol) and DIEA (13 mg, 0.1 mmol) in DMF (2 mL) was added (2-((5-chloro-2-((2-methoxy-4-(4-(piperazin-1-yl)piperidin-1-yl)phenyl)amino)pyrimidin-4-yl)amino)phenyl)dimethylphosphine oxide (20 mg, 0.033 mmol). The resulting mixture was stirred at room temperature for 1 h. The reaction was directly purified with pre-HPLC to give the title product (5 mg, 15%). ¹H NMR (400 MHz, DMSO) δ_{H} 11.18 (s, 1H), 11.09 (s, 1H), 8.48 (s, 1H), 8.07 (d, J=5.5 Hz, 2H), 7.67 (s, 1H), 7.52 (d, J=13.7 Hz, 1H), 7.35 (s, 4H), 7.09 (s, 1H), 6.63 (s, 1H), 6.47 (d, J=8.5 Hz, 1H), 5.07 (d, J=7.3 Hz, 1H), 3.76 (s, 5H), 3.44 (s, 8H), 2.88 (s, 1H), 2.72-2.52 (m, 12H), 2.39 (s, 5H), 2.01 (s, 1H), 1.83 (s, 2H), 1.76 (d, J=13.5 Hz, 6H), 1.54 (d, J=10.9 Hz, 2H); [M+H]⁺=966.4.

### Example 113: 5-(4-((4-(4-(1-(4-((5-chloro-4-((2-(dimethylphosphoryl)phenyl)amino)pyrimidin-2-yl)amino)-3-methoxyphenyl)piperidin-4-yl)piperazine-1-carbonyl)piperidin-1-yl)methyl)piperidin-1-yl)-2-(2,6-dioxopiperidin-3-yl)isoindoline-1,3-dione

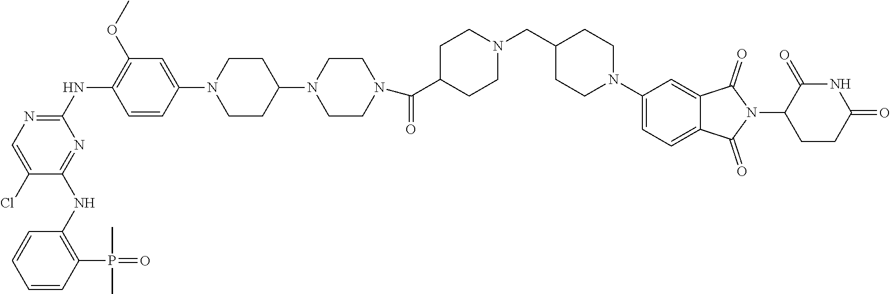 [SMILES: COc1cc(N2CCC(N3CCN(C(=O)C4CCN(CC5CCN(c6ccc7c(c6)C(=O)N(C6CCC(=O)NC6=O)C7=O)CC5)CC4)CC3)CC2)ccc1Nc1ncc(Cl)c(Nc2ccccc2P(C)(C)=O)n1]

The titled compound was synthesized in the procedures similar to Example 63. ¹H NMR (400 MHz, DMSO) δ_{H} 11.18 (s, 1H), 11.09 (s, 1H), 8.48 (s, 1H), 8.30 (s, 1H), 8.07 (d, J=6.9 Hz, 2H), 7.65 (d, J=8.3 Hz, 1H), 7.58-7.48 (m, 1H), 7.33 (d, J=23.7 Hz, 3H), 7.23 (d, J=8.6 Hz, 1H), 7.09 (s, 1H), 6.63 (s, 1H), 6.47 (d, J=8.0 Hz, 1H), 5.07 (d, J=7.6 Hz, 1H), 4.04 (d, J=12.6 Hz, 2H), 3.74 (d, J=13.8 Hz, 5H), 3.46 (s, 4H), 3.02-2.79 (m, 6H), 2.66 (s, 5H), 2.39 (s, 3H), 2.11 (s, 2H), 2.02 (s, 1H), 1.95-1.73 (m, 14H), 1.56 (s, 6H), 1.12 (d, J=11.1 Hz, 2H); [M+H]⁺=1034.5.

### Example 114: 5-(4-(5-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-5-oxopentyl)piperazin-1-yl)-2-(2,6-dioxopiperidin-3-yl)isoindoline-1,3-dione

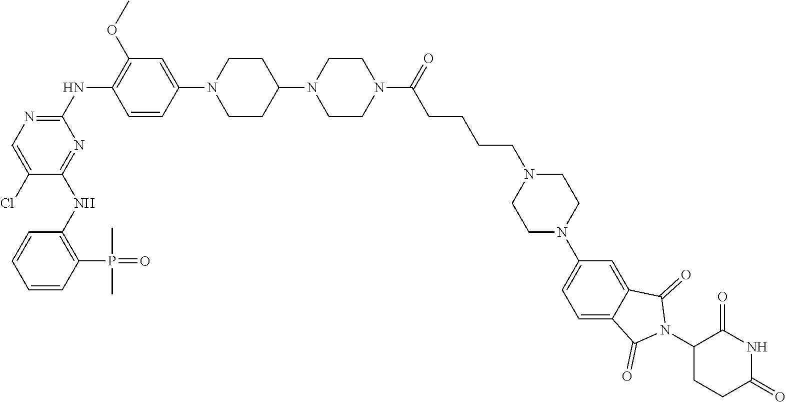 [SMILES: COc1cc(N2CCC(N3CCN(C(=O)CCCCN4CCN(c5ccc6c(c5)C(=O)N(C5CCC(=O)NC5=O)C6=O)CC4)CC3)CC2)ccc1Nc1ncc(Cl)c(Nc2ccccc2P(C)(C)=O)n1]

The titled compound was synthesized in the procedures similar to Example 62. ¹H NMR (400 MHz, DMSO) δ_{H} 11.18 (s, 1H), 11.09 (s, 1H), 8.48 (s, 1H), 8.07 (d, J=6.7 Hz, 2H), 7.68 (d, J=8.5 Hz, 1H), 7.53 (dd, J=13.8, 7.5 Hz, 1H), 7.34 (s, 3H), 7.26 (d, J=8.0 Hz, 1H), 7.09 (s, 1H), 6.62 (s, 1H), 6.46 (d, J=8.8 Hz, 1H), 5.07 (d, J=7.9 Hz, 1H), 3.73 (d, J=17.2 Hz, 5H), 3.44 (s, 8H), 2.86 (d, J=12.0 Hz, 1H), 2.70-2.55 (m, 4H), 2.39 (s, 8H), 2.35 (d, J=20.2 Hz, 5H), 2.01 (s, 1H), 1.83 (d, J=11.6 Hz, 2H), 1.76 (d, J=13.6 Hz, 6H), 1.51 (s, 6H); [M+H]⁺=994.5.

### Example 115: 1-(4-(4-((4-(4-((5-chloro-4-((2-(dimethylphosphoryl)phenyl)amino)pyrimidin-2-yl)amino)-3-methoxyphenyl)piperazin-1-yl)methyl)piperidin-1-yl)phenyl)dihydropyrimidine-2,4(1H,3H)-dione

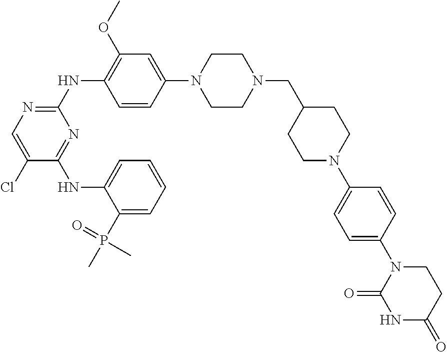 [SMILES: COc1cc(N2CCN(CC3CCN(c4ccc(N5CCC(=O)NC5=O)cc4)CC3)CC2)ccc1Nc1ncc(Cl)c(Nc2ccccc2P(C)(C)=O)n1]

The titled compound was synthesized in the procedures similar to Example 79. ¹H NMR (400 MHz, DMSO) δ_{H} 11.19 (s, 1H), 10.27 (s, 1H), 8.50 (s, 1H), 8.08 (d, J=10.6 Hz, 2H), 7.54 (s, 1H), 7.38 (d, J=20.9 Hz, 2H), 7.14 (d, J=8.5 Hz, 3H), 6.94 (d, J=7.5 Hz, 2H), 6.66 (s, 1H), 6.49 (s, 1H), 3.77 (s, 3H), 3.69 (s, 4H), 3.16 (s, 4H), 2.72-2.53 (m, 9H), 2.33-2.18 (m, 1H), 1.87-1.73 (m, 9H), 1.26 (s, 2H); [M+H]⁺=772.3.

### Example 116: 5-(3-(4-(4-((5-chloro-4-((2-(dimethylphosphoryl)phenyl)amino)pyrimidin-2-yl)amino)-3-methoxyphenyl)piperazin-1-yl)azetidine-1-carbonyl)-N-(4-(2,6-dioxopiperidin-3-yl)phenyl)pyrazine-2-carboxamide

### Step 1: 4-(2,6-bis(benzyloxy)pyridin-3-yl) aniline

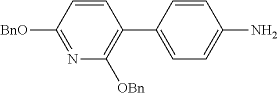 [SMILES: Nc1ccc(-c2ccc(OCc3ccccc3)nc2OCc2ccccc2)cc1]

A mixture of 4-bromoaniline (1.0 g, 5.81 mmol), 2,6-bis(benzyloxy)-3-(4,4,5,5-tetramethyl-1,3,2-dioxaborolan-2-yl)pyridine (2.67 g, 6.39 mmol), Pd(dppf)Cl₂ (425 mg, 0.581 mmol) and Cs₂CO₃ (4.72 g, 14.53 mmol) in dioxane (10 mL) and H₂O (2 mL) was stirred in a flask at 100° C. overnight under N₂. The mixture was evaporated in vacuum to afford the crude product, which was further purified with silica gel column chromatography (PE: EA=100: 0˜ 0: 100 gradient elution) to give the product (1.2 g, 55%). [M+H]⁺=383.4.

### Step 2: 3-(4-aminophenyl)piperidine-2,6-dione

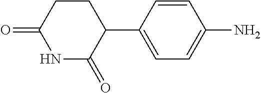 [SMILES: Nc1ccc(C2CCC(=O)NC2=O)cc1]

Under N₂, to a solution of 4-(2,6-bis(benzyloxy)pyridin-3-yl) aniline (1.2 g, 3.14 mmol) in MeOH (20 mL) and DCM (20 mL) was added 10% Pd/C (600 mg) at 25° C. And then the mixture was exchanged with H₂ two times and stirred under H₂ atmosphere at 25° C. for 24 h. Reaction was monitored by LCMS. The mixture was filtered through a pad of Celite and washed with MeOH (20 mL) and DCM (30 mL). The filtrate was concentrated under vacuum to obtain the product, which was further purified with silica gel column chromatography (DCM: MeOH=100: 0˜10: 1 gradient elution) to give the product (350 mg, 55%). [M+H]⁺=205.0.

### Step 3: 5-((4-(2,6-dioxopiperidin-3-yl)phenyl)carbamoyl)pyrazine-2-carboxylic acid

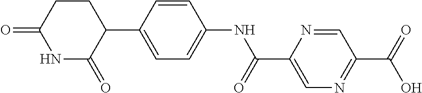 [SMILES: O=C1CCC(c2ccc(NC(=O)c3cnc(C(=O)O)cn3)cc2)C(=O)N1]

To a solution of pyrazine-2,5-dicarboxylic acid (150 mg, 0.892 mmol), HATU (373 mg, 0.981 mmol) and DIEA (345 mg, 2.676 mmol) in DMF (6 mL) was added 3-(4-aminophenyl)piperidine-2,6-dione (182 mg, 0.892 mmol). The resulting mixture was stirred at room temperature for 5 h. The reaction was quenched with water and extracted with DCM (2×40 mL). The combined organic layers were washed with brine, dried over anhydrous Na₂SO₄, and evaporated in vacuum to afford the crude product, which was further purified with silica gel column chromatography (DCM: MeOH=100: 0˜10: 1 gradient elution) to give the product (155 mg, 49%). [M+H]⁺=355.1.

### Step 4: tert-butyl 3-(4-(3-methoxy-4-nitrophenyl)piperazin-1-yl)azetidine-1-carboxylate

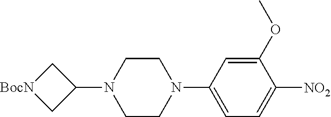 [SMILES: COc1cc(N2CCN(C3CN(C(=O)OC(C)(C)C)C3)CC2)ccc1[N+](=O)[O-]]

A mixture of 4-fluoro-2-methoxy-1-nitrobenzene (500 mg, 2.92 mmol), tert-butyl 3-(piperazin-1-yl)azetidine-1-carboxylate (706 mg, 2.92 mmol) and K₂CO₃ (800 mg, 5.79 mmol) in DMF (10 mL) was stirred in a flask at 80° C. overnight. The reaction was cooled to room temperature, the mixture was poured into water (50 mL) and stirred for 10 mins. The solid was filtered and washed with water (20 mL×2), dried to give the product (1.0 g, 87%). [M+H]⁺=393.3.

### Step 5: tert-butyl 3-(4-(4-amino-3-methoxyphenyl)piperazin-1-yl)azetidine-1-carboxylate

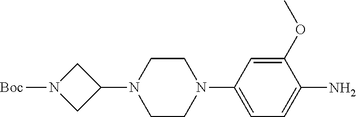 [SMILES: COc1cc(N2CCN(C3CN(C(=O)OC(C)(C)C)C3)CC2)ccc1N]

Under N₂, to a solution of tert-butyl 3-(4-(3-methoxy-4-nitrophenyl)piperazin-1-yl)azetidine-1-carboxylate (1.0 g, 2.55 mmol) in MeOH (20 mL) was added 10% Pd/C (200 mg) at 25° C. And then the mixture was exchanged with H₂ two times and stirred under H₂ atmosphere at 25° C. for 12 h. Reaction was monitored by LCMS. The mixture was filtered through a pad of Celite and washed with MeOH (30 mL). The filtrate was concentrated under vacuum to obtain the product (0.9 g, 98%). [M+H]⁺=363.4.

### Step 6: tert-butyl 3-(4-(4-((5-chloro-4-((2-(dimethylphosphoryl)phenyl)amino)pyrimidin-2-yl)amino)-3-methoxyphenyl)piperazin-1-yl)azetidine-1-carboxylate

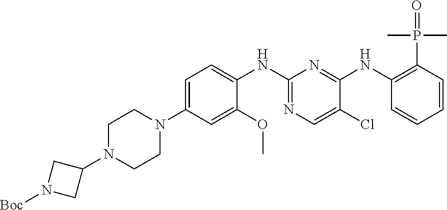 [SMILES: COc1cc(N2CCN(C3CN(C(=O)OC(C)(C)C)C3)CC2)ccc1Nc1ncc(Cl)c(Nc2ccccc2P(C)(C)=O)n1]

A mixture of (2-((2,5-dichloropyrimidin-4-yl)amino)phenyl)dimethylphosphine oxide (200 mg, 0.633 mmol), tert-butyl 3-(4-(4-amino-3-methoxyphenyl)piperazin-1-yl)azetidine-1-carboxylate (230 mg, 0.633 mmol), Pd₂(dba)₃ (60 mg, 0.063 mmol), BINAP (80 mg, 0.126 mmol) and K₃PO₄ (403 mg, 1.90 mmol) in toluene (10 mL) was stirred in a flask at 100° C. overnight under N₂. The mixture was evaporated in vacuum to afford the crude product, which was further purified with silica gel column chromatography (DCM: MeOH=100: 0˜ 10: 1 gradient elution) to give the product (160 mg, 39%). [M+H]⁺=642.6.

### Step 7: (2-((2-((4-(4-(azetidin-3-yl)piperazin-1-yl)-2-methoxyphenyl)amino)-5-chloropyrimidin-4-yl)amino)phenyl)dimethylphosphine oxide hydrochloride

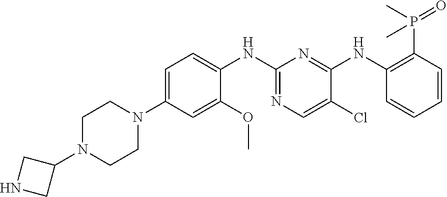 [SMILES: COc1cc(N2CCN(C3CNC3)CC2)ccc1Nc1ncc(Cl)c(Nc2ccccc2P(C)(C)=O)n1]

A solution of tert-butyl 3-(4-(4-((5-chloro-4-((2-(dimethylphosphoryl)phenyl)amino)pyrimidin-2-yl)amino)-3-methoxyphenyl)piperazin-1-yl)azetidine-1-carboxylate (160 mg, 0.249 mmol) in HC/1,4-dioxane (6 mL) was stirred in a flask at room temperature for 2 h. The mixture was evaporated in vacuum to afford the crude product (140 mg, 97%), which was used for next step without further purification. [M+H]⁺=542.5.

### Step 8: 5-(3-(4-(4-((5-chloro-4-((2-(dimethylphosphoryl)phenyl)amino)pyrimidin-2-yl)amino)-3-methoxyphenyl)piperazin-1-yl)azetidine-1-carbonyl)-N-(4-(2,6-dioxopiperidin-3-yl)phenyl)pyrazine-2-carboxamide

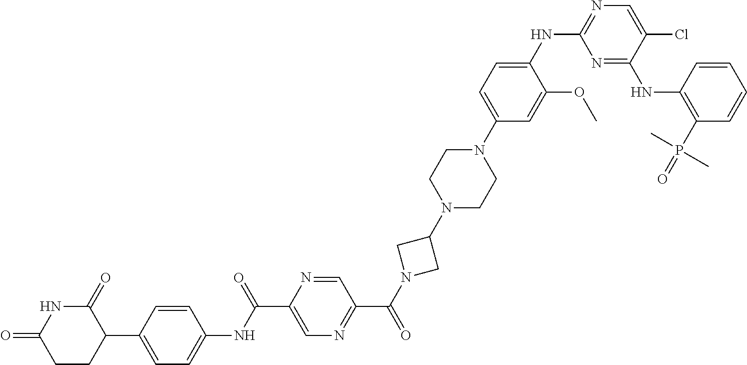 [SMILES: COc1cc(N2CCN(C3CN(C(=O)c4cnc(C(=O)Nc5ccc(C6CCC(=O)NC6=O)cc5)cn4)C3)CC2)ccc1Nc1ncc(Cl)c(Nc2ccccc2P(C)(C)=O)n1]

To a solution of 5-((4-(2,6-dioxopiperidin-3-yl)phenyl)carbamoyl)pyrazine-2-carboxylic acid (30.6 mg, 0.086 mmol), HATU (36 mg, 0.095 mmol) and DIEA (33 mg, 0.258 mmol) in DMF (3 mL) was added (2-((2-((4-(4-(azetidin-3-yl)piperazin-1-yl)-2-methoxyphenyl)amino)-5-chloropyrimidin-4-yl)amino)phenyl)dimethylphosphine oxide hydrochloride (50 mg, 0.086 mmol). The resulting mixture was stirred at room temperature for 5 h. The reaction was directly purified with pre-HPLC to give the title product (6 mg, 8%). ¹H NMR (400 MHz, DMSO) δ 11.19 (s, 1H), 10.92 (s, 1H), 10.86 (s, 1H), 9.26 (d, J=36.5 Hz, 1H), 8.48 (s, 1H), 8.09 (d, J=17.0 Hz, 2H), 7.86 (d, J=7.1 Hz, 2H), 7.52 (d, J=13.7 Hz, 1H), 7.43-7.30 (m, 2H), 7.24 (d, J=7.2 Hz, 2H), 7.10 (s, 1H), 6.66 (s, 1H), 6.49 (d, J=8.0 Hz, 1H), 4.69 (s, 1H), 4.49 (s, 1H), 4.23 (s, 1H), 4.05 (s, 1H), 3.85 (d, J=8.0 Hz, 1H), 3.77 (s, 3H), 3.30-3.25 (m, 2H), 3.19 (s, 4H), 2.68 (s, 1H), 2.54 (s, 4H), 2.21 (s, 1H), 2.06 (s, 1H), 1.76 (d, J=13.5 Hz, 6H); [M+H]⁺=878.5.

### Example 117: (2S,4R)—N—((S)-3-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide

### Step 1: tert-butyl (S)-(3-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-1-(4-(4-methylthiazol-5-yl)phenyl)-3-oxopropyl)carbamate

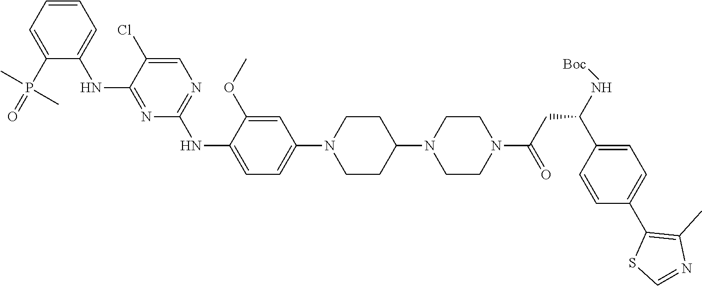

To a solution of (S)-3-((tert-butoxycarbonyl)amino)-3-(4-(4-methylthiazol-5-yl)phenyl)propanoic acid (The synthesis of this intermediate is described in J. Med. Chem., 2019, 62(2), 941-964) (366 mg, 1.05 mmol), HATU (441 mg, 1.16 mmol) and DIEA (406 mg, 3.15 mmol) in DMF (6 mL) was added (2-((5-chloro-2-((2-methoxy-4-(4-(piperazin-1-yl)piperidin-1-yl)phenyl)amino)pyrimidin-4-yl)amino)phenyl)dimethylphosphine oxide (600 mg, 1.05 mmol). The resulting mixture was stirred at room temperature for 3 h. The reaction was quenched with water and extracted with DCM (2×50 mL). The combined organic layers were washed with brine, dried over anhydrous Na₂SO₄, and evaporated in vacuum to afford the crude product, which was further purified with silica gel column chromatography (DCM: MeOH=100: 0˜10: 1 gradient elution) to give the product (340 mg, 36%). [M+H]⁺=914.7.

### Step 2: (S)-3-amino-1-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-3-(4-(4-methylthiazol-5-yl)phenyl)propan-1-one hydrochloride

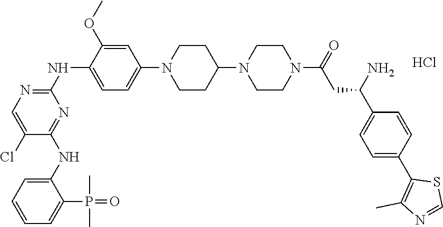 [SMILES: COc1cc(N2CCC(N3CCN(C(=O)C[C@H](N)c4ccc(-c5scnc5C)cc4)CC3)CC2)ccc1Nc1ncc(Cl)c(Nc2ccccc2P(C)(C)=O)n1 ; Cl]

A solution of tert-butyl tert-butyl (S)-(3-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-1-(4-(4-methylthiazol-5-yl)phenyl)-3-oxopropyl)carbamate (340 mg, 0.377 mmol) in HCl/1,4-dioxane (6 mL) was stirred in a flask at room temperature for 2 h. The mixture was evaporated in vacuum to afford the crude product (300 mg, 95%), which was used for next step without further purification. [M+H]⁺=814.7.

### Step 3: (2S,4R)—N—((S)-3-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-1-(4-(4-methylthiazol-5-yl)phenyl)-3-oxopropyl)-4-hydroxy-1-((R)-3-methyl-2-(3-methylisoxazol-5-yl)butanoyl)pyrrolidine-2-carboxamide

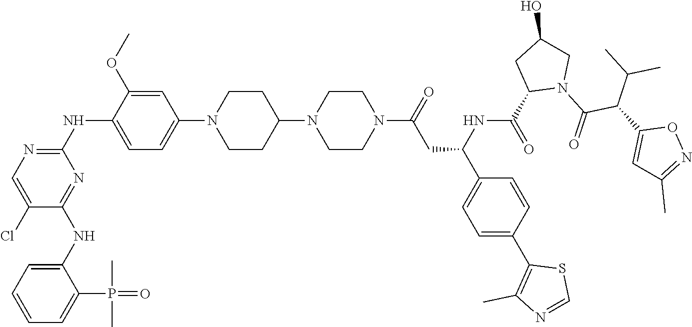 [SMILES: COc1cc(N2CCC(N3CCN(C(=O)C[C@H](NC(=O)[C@@H]4C[C@@H](O)CN4C(=O)[C@@H](c4cc(C)no4)C(C)C)c4ccc(-c5scnc5C)cc4)CC3)CC2)ccc1Nc1ncc(Cl)c(Nc2ccccc2P(C)(C)=O)n1]

To a solution of (2S,4R)-4-hydroxy-1-((R)-3-methyl-2-(3-methylisoxazol-5-yl)butanoyl)pyrrolidine-2-carboxylic acid (This intermediate can be prepared according to way described in WO2020010204) (7.1 mg, 0.024 mmol), HATU (10 mg, 0.026 mmol) and DIEA (10 mg, 0.078 mmol) in DMF (2 mL) was added (S)-3-amino-1-(4-(1-(4-((5-chloro-4-((2-(dimethylphosphoryl)phenyl)amino)pyrimidin-2-yl)amino)-3-methoxyphenyl)piperidin-4-yl)piperazin-1-yl)-3-(4-(4-methylthiazol-5-yl)phenyl)propan-1-one hydrochloride (20 mg, 0.024 mmol). The resulting mixture was stirred at room temperature for 5 h. The reaction was directly purified with pre-HPLC to give the title product (2.7 mg, 10%). ¹H NMR (400 MHz, DMSO) δ 11.16 (s, 1H), 8.98 (d, J=3.2 Hz, 1H), 8.45 (s, 2H), 8.07 (s, 1H), 8.05 (s, 1H), 7.43 (d, J=8.4 Hz, 2H), 7.39-7.33 (m, 4H), 7.07 (s, 1H), 6.57 (s, 1H), 6.42 (s, 1H), 6.20 (s, 1H), 5.19 (s, 1H), 5.08 (d, J=3.9 Hz, 1H), 4.31 (s, 1H), 4.27-4.24 (m, 1H), 3.74 (s, 4H), 3.64-3.60 (m, 1H), 2.83-2.79 (m, 2H), 2.55 (d, J=13.4 Hz, 6H), 2.46 (s, 4H), 2.32-2.30 (m, 1H), 2.26-2.23 (m, 2H), 2.18 (s, 3H), 2.16 (s, 1H), 1.74 (d, J=13.6 Hz, 12H), 1.47-1.39 (m, 3H), 0.95 (d, J=6.3 Hz, 4H), 0.80-0.77 (m, 1H), 0.75 (d, J=6.7 Hz, 3H); [M+H]⁺=1092.7.

### Example 118: 5-(3-(3-(4-(1-(4-((5-chloro-4-((2-(dimethylphosphoryl)phenyl)amino)pyrimidin-2-yl)amino)-3-methoxyphenyl)piperidin-4-yl)piperazin-1-yl)propyl)azetidin-1-yl)-2-(2,6-dioxopiperidin-3-yl)isoindoline-1,3-dione

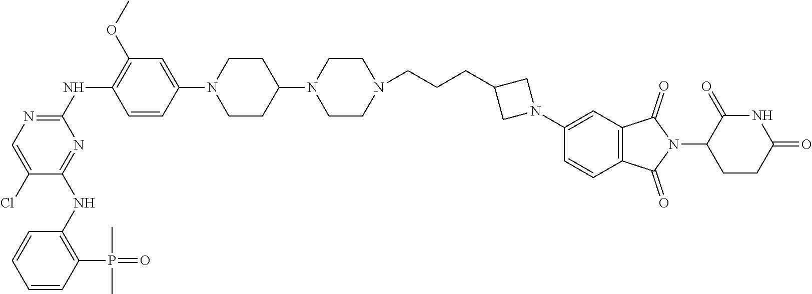 [SMILES: COc1cc(N2CCC(N3CCN(CCCC4CN(c5ccc6c(c5)C(=O)N(C5CCC(=O)NC5=O)C6=O)C4)CC3)CC2)ccc1Nc1ncc(Cl)c(Nc2ccccc2P(C)(C)=O)n1]

The titled compound was synthesized in the procedures similar to Example 56. ¹H NMR (400 MHz, DMSO) δ_{H} 11.18 (s, 1H), 11.07 (s, 1H), 8.48 (s, 1H), 8.07 (s, 2H), 7.63 (s, 1H), 7.57-7.47 (m, 1H), 7.42-7.29 (m, 2H), 7.09 (s, 1H), 6.76 (s, 1H), 6.63 (s, 2H), 6.47 (d, J=8.5 Hz, 1H), 5.05 (d, J=7.4 Hz, 1H), 4.13 (s, 2H), 3.74 (d, J=15.0 Hz, 5H), 3.65 (s, 2H), 2.93-2.52 (m, 9H), 2.31 (d, J=17.5 Hz, 7H), 2.02 (s, 1H), 1.87 (s, 2H), 1.76 (d, J=13.4 Hz, 6H), 1.65-1.42 (m, 7H); [M+H]⁺=923.4.

### Example 120: N-(2-((5-chloro-2-((4-(4-(1-(4-(2,6-dioxopiperidin-3-yl)phenethyl)piperidin-4-yl)piperazin-1-yl)-2-methoxyphenyl)amino)pyrimidin-4-yl)amino)phenyl)-N-methylmethanesulfonamide hydrochloride

### Step 1: tert-butyl 4-(4-(3-methoxy-4-nitrophenyl)piperazin-1-yl)piperidine-1-carboxylate

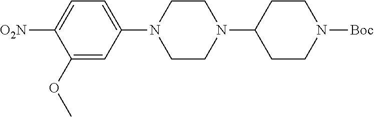 [SMILES: COc1cc(N2CCN(C3CCN(C(=O)OC(C)(C)C)CC3)CC2)ccc1[N+](=O)[O-]]

A mixture of 4-fluoro-2-methoxy-1-nitrobenzene (500 mg, 2.92 mmol), tert-butyl 4-(piperazin-1-yl)piperidine-1-carboxylate (940 mg, 3.49 mmol) and K₂CO₃ (800 mg, 5.79 mmol) in DMF (10 mL) was stirred in a flask at 80° C. overnight. The reaction was cooled to room temperature, the mixture was poured into water (50 mL) and stirred for 10 mins. The solid was filtered and washed with water (20 mL×2), dried to give the product (1.2 g, 98%). [M+H]⁺=421.3.

### Step 2: tert-butyl 4-(4-(4-amino-3-methoxyphenyl)piperazin-1-yl)piperidine-1-carboxylate

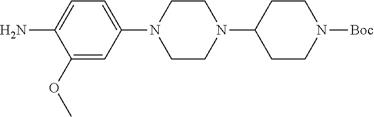 [SMILES: COc1cc(N2CCN(C3CCN(C(=O)OC(C)(C)C)CC3)CC2)ccc1N]

Under N₂, to a solution of tert-butyl 4-(4-(3-methoxy-4-nitrophenyl)piperazin-1-yl)piperidine-1-carboxylate (1.2 g, 2.85 mmol) in MeOH (30 mL) was added 10% Pd/C (200 mg) at 25° C. And then the mixture was exchanged with H₂ two times and stirred under H₂ atmosphere at 25° C. for 2 h. Reaction was monitored by HPLC. The mixture was filtered through a pad of Celite and washed with MeOH (30 mL). The filtrate was concentrated under vacuum to obtain the product (1.1 g, 98%). [M+H]⁺=391.3.

### Step 3: tert-butyl 4-(4-(4-((5-chloro-4-((2-(N-methylmethylsulfonamido)phenyl)amino)pyrimidin-2-yl)amino)-3-methoxyphenyl)piperazin-1-yl)piperidine-1-carboxylate

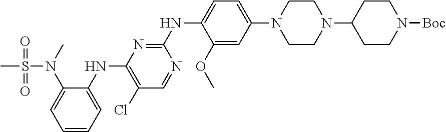 [SMILES: COc1cc(N2CCN(C3CCN(C(=O)OC(C)(C)C)CC3)CC2)ccc1Nc1ncc(Cl)c(Nc2ccccc2N(C)S(C)(=O)=O)n1]

A mixture of N-(2-((2,5-dichloropyrimidin-4-yl)amino)phenyl)-N-methylmethanesulfonamide (150 mg, 0.43 mmol) (The synthesis of this intermediate is described in J. Med. Chem., 2016, 59(16), 7478-7496), tert-butyl 4-(4-(4-amino-3-methoxyphenyl)piperazin-1-yl)piperidine-1-carboxylate (169 mg, 0.43 mmol), Pd₂(dba)₃ (40 mg, 0.043 mmol), BINAP (54 mg, 0.086 mmol) and K₃PO₄ (276 mg, 1.29 mmol) in toluene (10 mL) was stirred in a flask at 100° C. overnight under N₂. The mixture was evaporated in vacuum to afford the crude product, which was further purified with silica gel column chromatography (DCM: MeOH=100: 0˜ 10: 1 gradient elution) to give the product (160 mg, 53%). [M+H]⁺=701.3.

### Step 4: N-(2-((5-chloro-2-((2-methoxy-4-(4-(piperidin-4-yl)piperazin-1-yl)phenyl)amino)pyrimidin-4-yl)amino)phenyl)-N-methylmethanesulfonamide

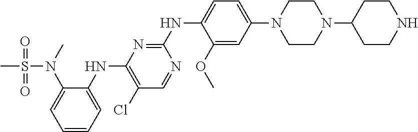 [SMILES: COc1cc(N2CCN(C3CCNCC3)CC2)ccc1Nc1ncc(Cl)c(Nc2ccccc2N(C)S(C)(=O)=O)n1]

A solution of tert-butyl 4-(4-(4-((5-chloro-4-((2-(N-methylmethylsulfonamido)phenyl)amino)pyrimidin-2-yl)amino)-3-methoxyphenyl)piperazin-1-yl)piperidine-1-carboxylate (160 mg, 0.23 mmol) in HCl/1,4-dioxane (10 mL) was stirred in a flask at room temperature for 2 h. The mixture was evaporated in vacuum to afford the crude product (135 mg, 98%), which was used for next step without further purification. [M+H]⁺=601.3.

### Step 5: N-(2-((5-chloro-2-((4-(4-(1-(4-(2,6-dioxopiperidin-3-yl)phenethyl)piperidin-4-yl)piperazin-1-yl)-2-methoxyphenyl)amino)pyrimidin-4-yl)amino)phenyl)-N-methylmethanesulfonamide hydrochloride

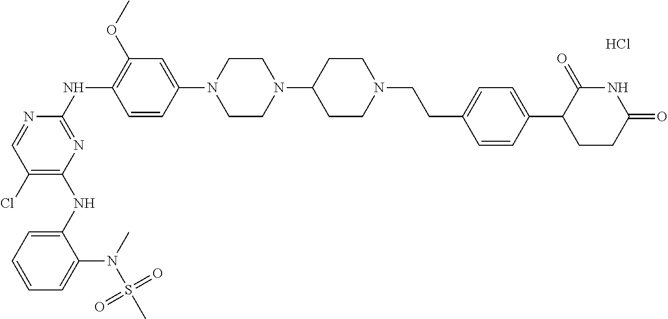 [SMILES: COc1cc(N2CCN(C3CCN(CCc4ccc(C5CCC(=O)NC5=O)cc4)CC3)CC2)ccc1Nc1ncc(Cl)c(Nc2ccccc2N(C)S(C)(=O)=O)n1 ; Cl]

A mixture of N-(2-((5-chloro-2-((2-methoxy-4-(4-(piperidin-4-yl)piperazin-1-yl)phenyl)amino)pyrimidin-4-yl)amino)phenyl)-N-methylmethanesulfonamide (25 mg, 0.04 mmol), 2-(4-(2,6-dioxopiperidin-3-yl)phenyl)acetaldehyde (11 mg, 0.048 mmol) and NaOAc (10 mg, 0.12 mmol) in DCM (3 mL) and MeOH (0.5 mL) was stirred in a flask at room temperature for 1 hour. The mixture was added NaBH₃CN (7 mg, 0.12 mmol) and stirred in a flask at room temperature for 2h. The reaction was quenched with water (10 mL) and extracted with DCM (10 mL×2). The combined organic layers were dried over anhydrous Na₂SO₄, and evaporated in vacuum to afford the crude product, which was further purified with pre-HPLC. Before lyophilization, the solution was treated with 1N HCl (0.2 mL) to get the product as HCl salt (9 mg, 22%). ¹H NMR (400 MHz, DMSO) δ_{H} 11.40 (s, 1H), 10.85 (s, 1H), 10.68 (s, 1H), 8.86-8.66 (m, 1H), 8.21 (s, 1H), 8.10 (s, 1H), 7.63 (d, J=7.1 Hz, 1H), 7.42-7.14 (m, 7H), 6.73 (s, 1H), 6.54 (s, 1H), 3.89 (s, 3H), 3.79 (s, 4H), 3.64 (s, 2H), 3.51 (s, 1H), 3.34-3.16 (m, 9H), 3.10-2.95 (m, 8H), 2.67 (s, 1H), 2.54 (s, 1H), 2.47-2.42 (m, 2H), 2.20 (d, J=12.6 Hz, 3H), 2.03 (s, 1H); [M+H]⁺=816.3.

### Example 119: N-(2-((5-chloro-2-((4-(4-(4-(4-(2,6-dioxopiperidin-3-yl)phenethyl)piperazin-1-yl)piperidin-1-yl)-2-methoxyphenyl)amino)pyrimidin-4-yl)amino)phenyl)-N-methylmethane sulfonamide

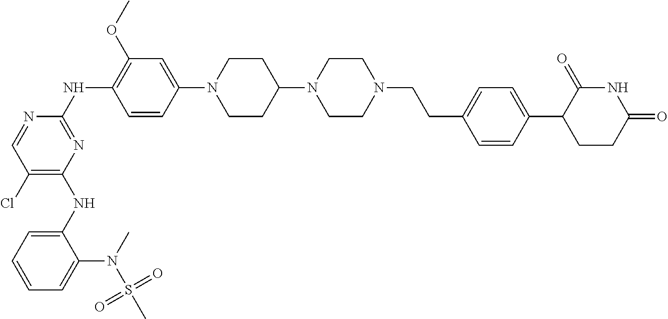 [SMILES: COc1cc(N2CCC(N3CCN(CCc4ccc(C5CCC(=O)NC5=O)cc4)CC3)CC2)ccc1Nc1ncc(Cl)c(Nc2ccccc2N(C)S(C)(=O)=O)n1]

To a solution of N-(2-((5-chloro-2-((2-methoxy-4-(4-(piperazin-1-yl)piperidin-1-yl)phenyl)amino)pyrimidin-4-yl)amino)phenyl)-N-methylmethanesulfonamide (30 mg, 0.05 mmol) and 2-(4-(2,6-dioxopiperidin-3-yl)phenyl)acetaldehyde (23 mg, 0.1 mmol) in DCM (5 mL) was added AcONa (8.2 mg, 0.1 mmol). The resulting mixture was stirred for 30 min at room temperature, then NaBH₃CN (6.2 mg, 0.1 mmol) was added. The resulting mixture was stirred for 2 h at room temperature. The crude solution was purified with Prep-HPLC column chromatography to give the title product (10 mg, 24%). ¹H NMR (400 MHz, DMSO) δ 10.82 (s, 1H), 8.28 (s, 2H), 8.21 (s, 1H), 8.10 (s, 1H), 7.59 (d, J=8.2 Hz, 1H), 7.36 (d, J=8.6 Hz, 1H), 7.25 (s, 1H), 7.15 (dd, J=26.3, 7.7 Hz, 5H), 6.62 (s, 1H), 6.46 (d, J=8.8 Hz, 1H), 3.81 (d, J=7.0 Hz, 1H), 3.75 (s, 3H), 3.71 (s, 1H), 3.18 (s, 3H), 3.10 (s, 3H), 2.76-2.59 (m, 6H), 2.51 (s, 6H), 2.48-2.40 (m, 4H), 2.33 (s, 2H), 2.17 (d, J=11.1 Hz, 1H), 2.03 (s, 1H), 1.86 (d, J=10.2 Hz, 2H), 1.53 (d, J=10.6 Hz, 2H); [M+H]⁺=816.3.

### Example 121: N-(2-((5-chloro-2-((4-(4-(4-(2-(1-(4-(2,6-dioxopiperidin-3-yl)phenyl)piperidin-4-yl)ethyl)piperazin-1-yl)piperidin-1-yl)-2-methoxyphenyl)amino)pyrimidin-4-yl)amino)phenyl)-N-methylmethanesulfonamide

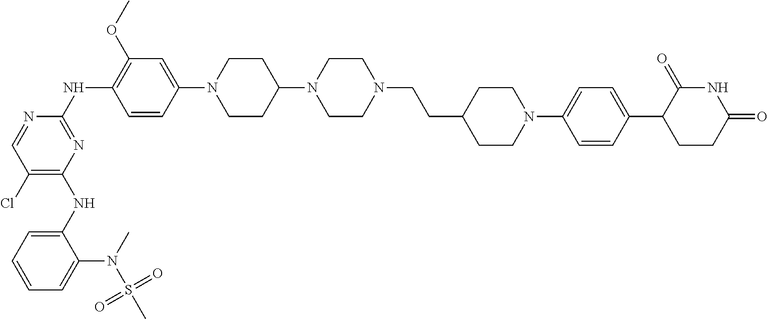 [SMILES: COc1cc(N2CCC(N3CCN(CCC4CCN(c5ccc(C6CCC(=O)NC6=O)cc5)CC4)CC3)CC2)ccc1Nc1ncc(Cl)c(Nc2ccccc2N(C)S(C)(=O)=O)n1]

The titled compound was synthesized in the procedures similar to Example 119. ¹H NMR (400 MHz, DMSO) δ 10.78 (s, 1H), 8.28 (s, 4H), 8.10 (s, 1H), 7.58 (d, J=7.7 Hz, 1H), 7.36 (d, J=8.4 Hz, 1H), 7.25 (t, J=7.7 Hz, 1H), 7.16 (t, J=7.6 Hz, 1H), 7.02 (d, J=8.2 Hz, 2H), 6.87 (d, J=8.2 Hz, 2H), 6.62 (s, 1H), 6.45 (d, J=8.9 Hz, 1H), 3.75 (s, 3H), 3.70 (s, 2H), 3.63 (d, J=11.8 Hz, 2H), 3.18 (s, 3H), 3.10 (s, 3H), 2.61 (dd, J=24.3, 12.4 Hz, 8H), 2.47-2.24 (m, 8H), 2.18-2.07 (m, 1H), 1.99-1.95 (m, 1H), 1.85 (d, J=11.5 Hz, 2H), 1.74 (d, J=11.7 Hz, 2H), 1.52-1.48 (m, 2H), 1.39 (s, 3H), 1.23 (d, J=9.0 Hz, 2H); [M+H]⁺=899.4.

### Example 122: N-(2-((5-chloro-2-((4-(4-(4-(2-(1-(4-(2,6-dioxopiperidin-3-yl)phenyl)piperidin-4-yl)acetyl)piperazin-1-yl)piperidin-1-yl)-2-methoxyphenyl)amino)pyrimidin-4-yl)amino)phenyl)-N-methylmethanesulfonamide

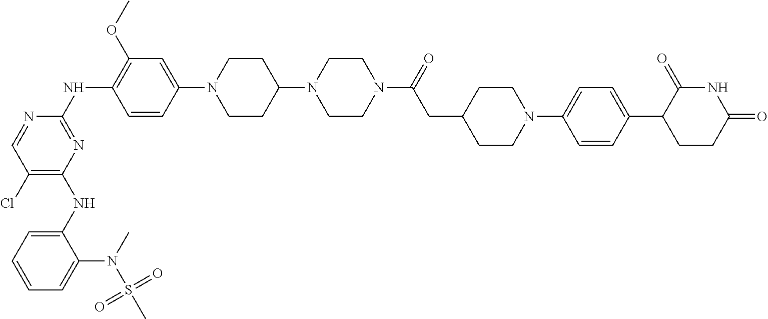 [SMILES: COc1cc(N2CCC(N3CCN(C(=O)CC4CCN(c5ccc(C6CCC(=O)NC6=O)cc5)CC4)CC3)CC2)ccc1Nc1ncc(Cl)c(Nc2ccccc2N(C)S(C)(=O)=O)n1]

The titled compound was synthesized in the procedures similar to Example 24. ¹H NMR (400 MHz, DMSO) δ 10.78 (s, 1H), 8.30 (d, J=13.2 Hz, 3H), 8.11 (d, J=9.5 Hz, 2H), 7.59 (d, J=8.3 Hz, 1H), 7.36 (d, J=8.9 Hz, 1H), 7.25 (s, 1H), 7.16 (t, J=7.8 Hz, 1H), 7.03 (d, J=8.5 Hz, 2H), 6.88 (d, J=8.0 Hz, 2H), 6.62 (s, 1H), 6.46 (d, J=8.6 Hz, 1H), 3.75 (s, 3H), 3.72 (s, 2H), 3.64 (d, J=12.1 Hz, 2H), 3.46 (s, 6H), 3.18 (s, 3H), 3.10 (s, 3H), 2.65-2.62 (m, 5H), 2.41-2.36 (m, 5H), 2.28 (d, J=6.3 Hz, 2H), 2.17-2.07 (m, 1H), 2.01 (dd, J=9.9, 7.0 Hz, 1H), 1.85 (d, J=10.9 Hz, 3H), 1.74 (d, J=11.9 Hz, 2H), 1.55 (d, J=12.6 Hz, 2H), 1.28 (d, J=11.1 Hz, 2H); [M+H]⁺=913.4.

### Example 123: N-(2-((5-chloro-2-((4-(4-(4-(3-(4-(4-(2,6-dioxopiperidin-3-yl)phenyl)piperidin-1-yl)propanoyl)piperazin-1-yl)piperidin-1-yl)-2-methoxyphenyl)amino)pyrimidin-4-yl)amino)phenyl)-N-methylmethanesulfonamide

### Step 1: tert-butyl 4-(4-(2,6-bis(benzyloxy)pyridin-3-yl)phenyl)piperidine-1-carboxylate

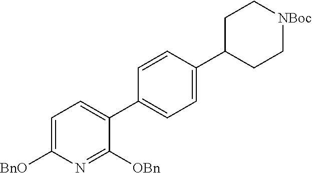 [SMILES: CC(C)(C)OC(=O)N1CCC(c2ccc(-c3ccc(OCc4ccccc4)nc3OCc3ccccc3)cc2)CC1]

Tert-butyl 4-(4-(4,4,5,5-tetramethyl-1,3,2-dioxaborolan-2-yl)phenyl)piperidine-1-carboxylate (17 g, 44 mmol), Pd(dppf)Cl₂ (3.2 g, 4.4 mmol), 2,6-bis(benzyloxy)-3-bromopyridine (16.2 g, 44.0 mmol), Cs₂CO₃ (28.7 g, 88 mmol) were placed in dioxane/water (300 mL, 10:1). The mixture was stirred at 100° C. for overnight until LC-MS indicated all the starting material was consumed. The resulting solution was filtered and the filtrate was concentrated to afford the crude residue which was purified by SiO₂-gel column (eluted with EtOAc/Hexane=1:1) to give the desire product (5 g, 21%). [M+H]⁺=551.3.

### Step 2: tert-butyl 4-(4-(2,6-dioxopiperidin-3-yl)phenyl)piperidine-1-carboxylate

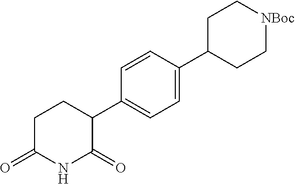 [SMILES: CC(C)(C)OC(=O)N1CCC(c2ccc(C3CCC(=O)NC3=O)cc2)CC1]

Tert-butyl 4-(4-(2,6-bis(benzyloxy)pyridin-3-yl)phenyl)piperidine-1-carboxylate (5 g, 9.1 mmol) was dissolved in MeOH (50 mL), Pd/C (10%, w/w, 0.5 g) was added to the solution in one portion. The resulting mixture was stirred under H₂ atmosphere for overnight until LC-MS indicated all the starting material was consumed. The resulting solution was filtered and the filtrate was concentrated to give the desire product (1.9 g, 56.1%). [M+H]⁺=373.

### Step 3: 3-(4-(piperidin-4-yl)phenyl)piperidine-2,6-dione hydrochloride

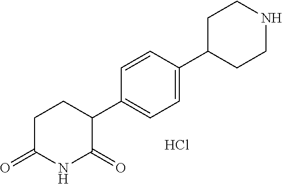 [SMILES: O=C1CCC(c2ccc(C3CCNCC3)cc2)C(=O)N1 ; Cl]

Tert-butyl 4-(4-(2,6-dioxopiperidin-3-yl)phenyl)piperidine-1-carboxylate (1.9 g, 5.1 mmol) was placed in HCl-dixoane (4M, 20 mL), the mixture was stirred at room temperature for 2h until LC-MS indicated all the starting material was consumed. The resulting solution was concentrated to afford the crude residue which was triturated with MTBE (5 mL) to afford desire product (1.38 g, 88%). [M+H]⁺=273.2.

### Step 4: tert-butyl 3-(4-(4-(2,6-dioxopiperidin-3-yl)phenyl)piperidin-1-yl)propanoate

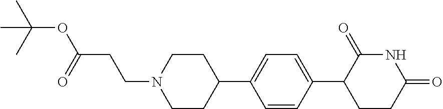 [SMILES: CC(C)(C)OC(=O)CCN1CCC(c2ccc(C3CCC(=O)NC3=O)cc2)CC1]

A mixture of 3-(4-(piperidin-4-yl)phenyl)piperidine-2,6-dione hydrochloride (228 mg, 0.74 mmol), tert-butyl acrylate (189 mg, 1.48 mmol) and DIEA (189 mg, 1.48 mmol) in MeCN (8 mL) was stirred in a flask at 80° C. overnight. The mixture was evaporated in vacuum to afford the crude product, which was further purified with silica gel column chromatography (DCM: MeOH=100: 0˜ 10: 1 gradient elution) to give the product (178 mg, 60%); [M+H]⁺=401.2.

### Step 5: 3-(4-(4-(2,6-dioxopiperidin-3-yl)phenyl)piperidin-1-yl)propanoic acid

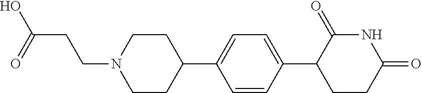 [SMILES: O=C(O)CCN1CCC(c2ccc(C3CCC(=O)NC3=O)cc2)CC1]

A solution of tert-butyl 3-(4-(4-(2,6-dioxopiperidin-3-yl)phenyl)piperidin-1-yl)propanoate (178 mg, 0.45 mmol) in HCl/1,4-dioxane (8 mL) was stirred in a flask at room temperature overnight. The mixture was evaporated in vacuum to afford the crude product (150 mg, 97%), which was used for next step without further purification. [M+H]⁺ 345.4.

### Step 6: N-(2-((5-chloro-2-((4-(4-(4-(3-(4-(4-(2,6-dioxopiperidin-3-yl)phenyl)piperidin-1-yl)propanoyl)piperazin-1-yl)piperidin-1-yl)-2-methoxyphenyl)amino)pyrimidin-4-yl)amino)phenyl)-N-methylmethanesulfonamide

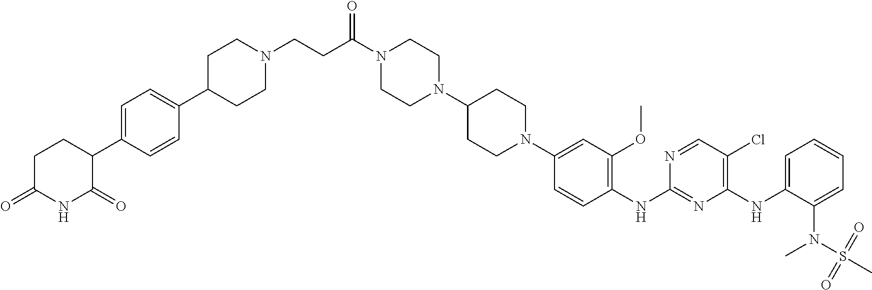 [SMILES: COc1cc(N2CCC(N3CCN(C(=O)CCN4CCC(c5ccc(C6CCC(=O)NC6=O)cc5)CC4)CC3)CC2)ccc1Nc1ncc(Cl)c(Nc2ccccc2N(C)S(C)(=O)=O)n1]

To a solution of 3-(4-(4-(2,6-dioxopiperidin-3-yl)phenyl)piperidin-1-yl)propanoic acid (20 mg, 0.06 mmol), HATU (46 mg, 0.12 mmol) and DIEA (15 mg, 0.12 mmol) in DMF (2 mL) was added N-(2-((5-chloro-2-((2-methoxy-4-(4-(piperazin-1-yl)piperidin-1-yl)phenyl)amino)pyrimidin-4-yl)amino)phenyl)-N-methylmethanesulfonamide (36 mg, 0.06 mmol). The resulting mixture was stirred at room temperature for 1 h. The reaction was directly purified with pre-HPLC to give the title product (10 mg, 18%); ¹H NMR (400 MHz, DMSO) δ 10.83 (s, 1H), 8.28 (s, 2H), 8.11 (d, J=7.9 Hz, 2H), 7.59 (d, J=7.2 Hz, 1H), 7.37 (d, J=8.4 Hz, 1H), 7.24-7.08 (m, 5H), 6.63 (s, 1H), 6.46 (d, J=8.4 Hz, 1H), 3.94-3.68 (m, 6H), 3.46 (s, 6H), 3.18 (s, 3H), 3.10 (s, 3H), 2.98 (d, J=11.0 Hz, 2H), 2.67 (t, J=11.3 Hz, 3H), 2.54 (s, 4H), 2.46 (s, 5H), 2.16 (d, J=11.5 Hz, 1H), 2.03 (t, J=11.1 Hz, 3H), 1.85 (d, J=10.5 Hz, 2H), 1.73 (d, J=11.5 Hz, 2H), 1.59-1.43 (m, 4H); [M+H]⁺=927.4.

### Example 124: N-(2-((5-chloro-2-((4-(4-(4-(4-(2,4-dioxotetrahydropyrimidin-1(2H)-yl) phenethyl)piperazin-1-yl)piperidin-1-yl)-2-methoxyphenyl)amino)pyrimidin-4-yl)amino)phenyl)-N-methylmethanesulfonamide

### Step 1: 4-(2,4-dioxotetrahydropyrimidin-1(2H)-yl)phenethyl acetate

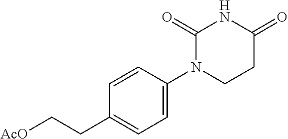 [SMILES: CC(=O)OCCc1ccc(N2CCC(=O)NC2=O)cc1]

2-(4-aminophenyl)ethan-1-ol (13.7 g, 100 mmol), acrylic acid (7.9 g, 110 mmol) were placed in Toluene (150 mL). The mixture was stirred at 90° C. for 4h until LC-MS indicated all the starting material was consumed. Cooling the reaction to room temperature, Urea (30 g, 500 mmol) and AcOH (150 mL) was added to the mixture. The resulting mixture was stirred at 110° C. for 24h until all the intermediate was consumed. The reaction was cooled to room temperature, concentrated under vacuo to remove excess solvents. The resulting mixture was diluted with EtOAc, adjust pH to 7 with saturated NaHCO₃ (aq.) solution, extracted with EtOAc. The combined organic phases were dried over Na₂SO₄, concentrated and purified with SiO₂-gel column (eluted with EtOAc/Hexane=1:1) to give the desired product (5.9 g, 21.4%). [M+H]⁺=277.1

### Step 2: 1-(4-(2-hydroxyethyl)phenyl)dihydropyrimidine-2.4(1H,3H)-dione

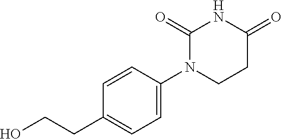 [SMILES: O=C1CCN(c2ccc(CCO)cc2)C(=O)N1]

4-(2,4-dioxotetrahydropyrimidin-1(2H)-yl)phenethyl acetate (5.9 g, 21.4 mmol) was dissolved in HCl (aq., 2 M, 50 mL). The solution was stirred at 100° C. for 1h until LC-MS indicated all the starting material was consumed. Concentrated to remove solvent, diluted with EtOAc, adjust pH to 7 with saturated NaHCO₃ (aq.) solution, extracted with EtOAc, the combined organic phase was washed with Brine, dried over Na₂SO₄, concentrated to give the desire product (3.1 g, 61.9%). [M+H]⁺=235.1.

### Step 3: 2-(4-(2,4-dioxotetrahydropyrimidin-1(2H)-yl)phenyl)acetaldehyde

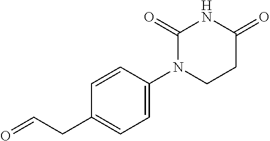 [SMILES: O=CCc1ccc(N2CCC(=O)NC2=O)cc1]

The titled compound was prepared in a manner similar to that in Example 79 step 7. [M+H]⁺=233.

### Step 4: N-(2-((5-chloro-2-((4-(4-(4-(4-(2,4-dioxotetrahydropyrimidin-1(2H)-yl)phenethyl)piperazin-1-yl)piperidin-1-yl)-2-methoxyphenyl)amino)pyrimidin-4-yl)amino)phenyl)-N-methylmethanesulfonamide

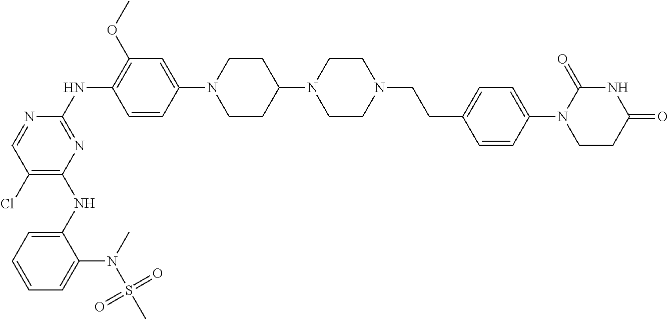 [SMILES: COc1cc(N2CCC(N3CCN(CCc4ccc(N5CCC(=O)NC5=O)cc4)CC3)CC2)ccc1Nc1ncc(Cl)c(Nc2ccccc2N(C)S(C)(=O)=O)n1]

The titled compound was prepared in a manner similar to that in Example 79 step 8. ¹H NMR (400 MHz, DMSO) δ 10.35 (s, 1H), 8.28 (s, 2H), 8.21-8.06 (m, 2H), 7.59 (d, J=7.9 Hz, 1H), 7.37 (d, J=8.0 Hz, 1H), 7.24 (s, 4H), 7.16 (t, J=7.6 Hz, 1H), 6.63 (s, 1H), 6.46 (d, J=9.1 Hz, 1H), 3.76 (s, 7H), 3.18 (s, 3H), 3.10 (s, 3H), 2.65-2.60 (m, 17H), 1.90 (d, J=10.4 Hz, 2H), 1.56 (d, J=11.1 Hz, 2H); [M+H]⁺=817.3.

### Example 125: N-(2-((5-chloro-2-((4-(4-(4-(2-(1-(4-(2,4-dioxotetrahydropyrimidin-1(2H)-yl)-3-fluorophenyl)piperidin-4-yl)ethyl)piperazin-1-yl)piperidin-1-yl)-2-methoxy phenyl)amino)pyrimidin-4-yl)amino)phenyl)-N-methylmethanesulfonamide

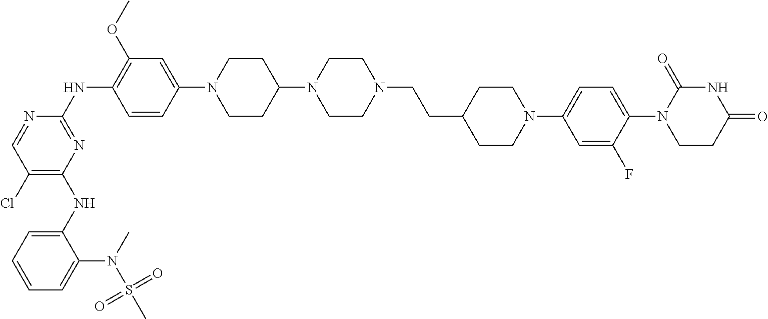 [SMILES: COc1cc(N2CCC(N3CCN(CCC4CCN(c5ccc(N6CCC(=O)NC6=O)c(F)c5)CC4)CC3)CC2)ccc1Nc1ncc(Cl)c(Nc2ccccc2N(C)S(C)(=O)=O)n1]

The titled compound was synthesized in the procedures similar to Example 79. ¹H NMR (400 MHz, DMSO) δ_{H} 10.38 (s, 1H), 8.28 (s, 2H), 8.11 (d, J=7.7 Hz, 2H), 7.58 (d, J=8.3 Hz, 1H), 7.36 (d, J=8.4 Hz, 1H), 7.25 (s, 1H), 7.16 (d, J=7.3 Hz, 2H), 6.77 (dd, J=20.3, 11.8 Hz, 2H), 6.62 (s, 1H), 6.45 (d, J=8.7 Hz, 1H), 3.73 (d, J=17.9 Hz, 7H), 3.61 (s, 2H), 3.18 (s, 3H), 3.10 (s, 3H), 2.72-2.62 (m, 7H), 2.52 (s, 2H), 2.45-2.25 (m, 6H), 1.84 (s, 2H), 1.73 (d, J=13.7 Hz, 2H), 1.60-1.33 (m, 6H), 1.21 (d, J=18.0 Hz, 3H); [M+H]⁺=918.4.

### Example 126: N-(2-((5-chloro-2-((4-(4-(4-(4-((2,6-dioxopiperidin-3-yl)amino)phenethyl)piperazin-1-yl)piperidin-1-yl)-2-methoxyphenyl)amino)pyrimidin-4-yl)amino)phenyl)-N-methylmethanesulfonamide

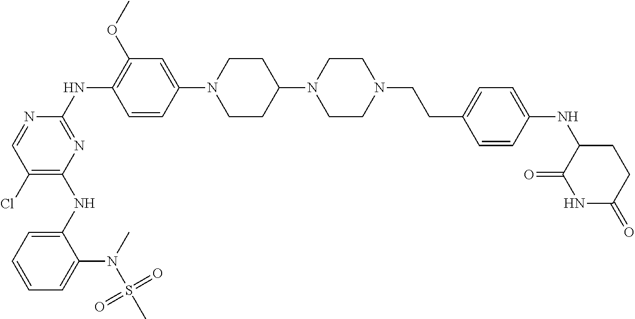 [SMILES: COc1cc(N2CCC(N3CCN(CCc4ccc(NC5CCC(=O)NC5=O)cc4)CC3)CC2)ccc1Nc1ncc(Cl)c(Nc2ccccc2N(C)S(C)(=O)=O)n1]

The titled compound was synthesized in the procedures similar to Example 48. ¹H NMR (400 MHz, DMSO) δ 10.78 (s, 1H), 8.26 (d, J=11.2 Hz, 3H), 8.11 (d, J=7.5 Hz, 2H), 7.59 (d, J=7.9 Hz, 1H), 7.36 (d, J=8.1 Hz, 1H), 7.25 (s, 1H), 7.16 (t, J=7.4 Hz, 1H), 6.93 (d, J=8.0 Hz, 2H), 6.67-6.55 (m, 3H), 6.45 (d, J=8.6 Hz, 1H), 5.65 (d, J=6.9 Hz, 1H), 4.27 (s, 1H), 3.75 (s, 3H), 3.71 (s, 1H), 3.18 (s, 3H), 3.10 (s, 3H), 2.79-2.62 (m, 3H), 2.56 (d, J=18.7 Hz, 7H), 2.46-2.27 (m, 7H), 2.09 (s, 1H), 1.86 (d, J=11.5 Hz, 3H), 1.52 (d, J=11.2 Hz, 2H); [M+H]⁺=831.3.

### Example 127: N-(2-((5-chloro-2-((4-(4-(4-(3-(4-((2,6-dioxopiperidin-3 yl)oxy)phenoxy)propyl) piperazin-1-yl)piperidin-1-yl)-2-methoxyphenyl)amino)pyrimidin-4-yl)amino)phenyl)-N-methylmethanesulfonamide

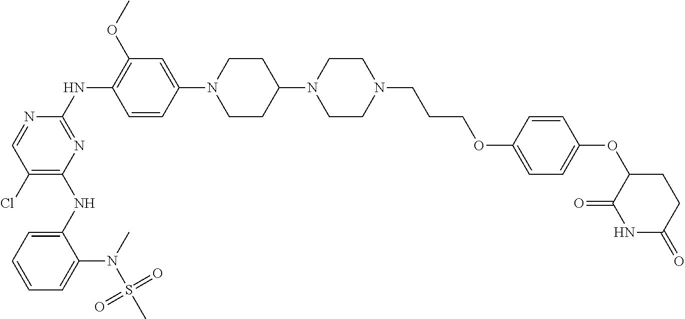 [SMILES: COc1cc(N2CCC(N3CCN(CCCOc4ccc(OC5CCC(=O)NC5=O)cc4)CC3)CC2)ccc1Nc1ncc(Cl)c(Nc2ccccc2N(C)S(C)(=O)=O)n1]

The titled compound was synthesized in the procedures similar to Example 92. ¹H NMR (400 MHz, DMSO) δ 10.91 (s, 1H), 8.28 (s, 2H), 8.11 (d, J=8.3 Hz, 2H), 7.58 (d, J=7.7 Hz, 1H), 7.36 (d, J=8.4 Hz, 1H), 7.25 (s, 1H), 7.16 (s, 1H), 6.94 (d, J=9.0 Hz, 2H), 6.85 (d, J=8.9 Hz, 2H), 6.62 (s, 1H), 6.45 (d, J=8.6 Hz, 1H), 5.04 (dd, J=10.4, 4.8 Hz, 1H), 3.94 (s, 2H), 3.73 (d, J=19.2 Hz, 5H), 3.18 (s, 3H), 3.10 (s, 3H), 2.65-2.57 (m, 8H), 2.36-2.26 (m, 7H), 2.13-2.02 (m, 2H), 1.84 (s, 4H), 1.53 (d, J=11.3 Hz, 2H); [M+H]⁺=862.2.

### Example 128: N-(2-((5-chloro-2-((4-(4-(4-(5-(4-(2-(2,6-dioxopiperidin-3-yl)-1,3-dioxoisoindolin-5-yl)piperazin-1-yl)pentanoyl)piperazin-1-yl)piperidin-1-yl)-2-methoxyphenyl)amino)pyrimidin-4-yl)amino)phenyl)-N-methylmethanesulfonamide

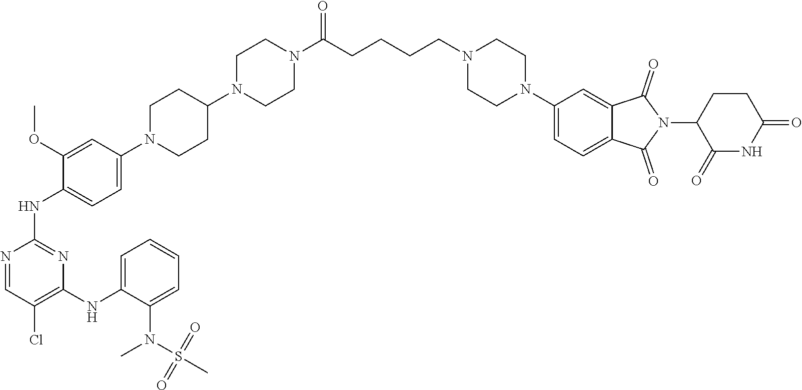 [SMILES: COc1cc(N2CCC(N3CCN(C(=O)CCCCN4CCN(c5ccc6c(c5)C(=O)N(C5CCC(=O)NC5=O)C6=O)CC4)CC3)CC2)ccc1Nc1ncc(Cl)c(Nc2ccccc2N(C)S(C)(=O)=O)n1]

The titled compound was synthesized in the procedures similar to Example 62. ¹H NMR (400 MHz, DMSO) δ 11.09 (s, 1H), 8.28 (s, 2H), 8.15 (s, 1H), 8.11 (d, J=7.4 Hz, 2H), 7.68 (d, J=8.1 Hz, 1H), 7.58 (d, J=7.7 Hz, 1H), 7.36 (d, J=10.3 Hz, 2H), 7.26 (d, J=8.3 Hz, 2H), 7.15 (t, J=7.3 Hz, 1H), 6.62 (s, 1H), 6.45 (d, J=8.8 Hz, 1H), 5.11-4.98 (m, 1H), 3.75 (s, 3H), 3.71 (s, 2H), 3.44 (s, 8H), 3.18 (s, 3H), 3.10 (s, 3H), 2.93-2.81 (m, 1H), 2.70-2.51 (m, 11H), 2.41-2.30 (m, 5H), 2.05-1.96 (m, 1H), 1.84 (d, J=11.3 Hz, 2H), 1.59-1.44 (m, 6H); [M+H]⁺=1025.4.

### Example 129: N-(2-((5-chloro-2-((4-(4-(4-(1-((1-(2-(2,6-dioxopiperidin-3-yl)-1,3-dioxoisoindolin-5-yl)piperidin-4-yl)methyl)piperidine-4-carbonyl)piperazin-1-yl)piperidin-1-yl)-2-methoxyphenyl)amino)pyrimidin-4-yl)amino)phenyl)-N-methylmethanesulfonamide

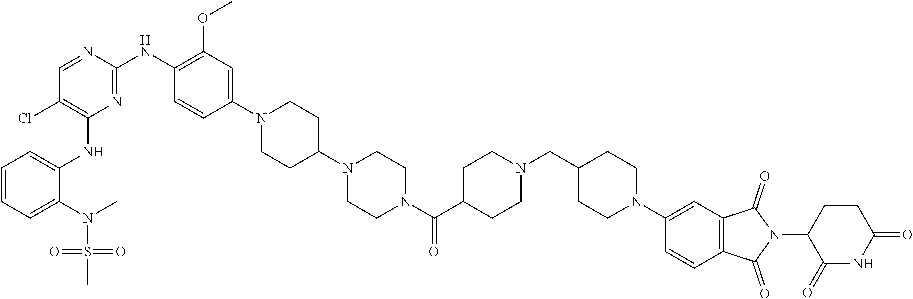 [SMILES: COc1cc(N2CCC(N3CCN(C(=O)C4CCN(CC5CCN(c6ccc7c(c6)C(=O)N(C6CCC(=O)NC6=O)C7=O)CC5)CC4)CC3)CC2)ccc1Nc1ncc(Cl)c(Nc2ccccc2N(C)S(C)(=O)=O)n1]

The titled compound was synthesized in the procedures similar to Example 63. ¹H NMR (400 MHz, DMSO) δ 11.09 (s, 1H), 8.28 (s, 2H), 8.12 (s, 1H), 8.10 (s, 1H), 7.65 (d, J=7.9 Hz, 1H), 7.59 (d, J=7.7 Hz, 1H), 7.36 (d, J=8.8 Hz, 1H), 7.30 (s, 1H), 7.23 (s, 2H), 7.17 (d, J=7.6 Hz, 1H), 6.62 (s, 1H), 6.46 (d, J=8.7 Hz, 1H), 5.06 (dd, J=13.1, 5.5 Hz, 1H), 4.04 (d, J=12.8 Hz, 2H), 3.75 (s, 3H), 3.72 (s, 2H), 3.45 (s, 4H), 3.18 (s, 3H), 3.10 (s, 3H), 3.03-2.92 (m, 3H), 2.85 (s, 3H), 2.74-2.53 (m, 6H), 2.42-2.30 (m, 2H), 2.11 (d, J=4.7 Hz, 2H), 2.02 (s, 1H), 1.84-1.76 (m, 8H), 1.56 (s, 6H), 1.12 (d, J=11.9 Hz, 2H); [M+H]⁺=1065.4.

### Example 130: N-(2-((5-chloro-2-((4-(4-(4-(2-(4-(2-(2,6-dioxopiperidin-3-yl)-3-oxoisoindolin-5-yl)piperazin-1-yl)acetyl)piperazin-1-yl)piperidin-1-yl)-2-methoxyphenyl)amino)pyrimidin-4-yl)amino)phenyl)-N-methylmethanesulfonamide

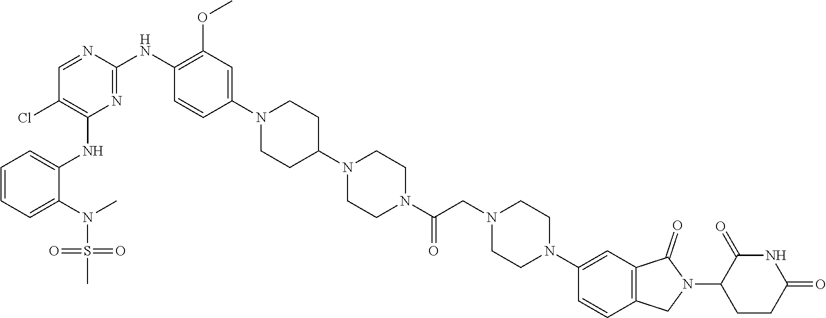 [SMILES: COc1cc(N2CCC(N3CCN(C(=O)CN4CCN(c5ccc6c(c5)C(=O)N(C5CCC(=O)NC5=O)C6)CC4)CC3)CC2)ccc1Nc1ncc(Cl)c(Nc2ccccc2N(C)S(C)(=O)=O)n1]

The titled compound was synthesized in the procedures similar to Example 68. ¹H NMR (400 MHz, DMSO) δ 10.70 (s, 1H), 8.28 (s, 2H), 8.11 (d, J=6.9 Hz, 2H), 7.55-7.50 (m, 2H), 7.36 (d, J=8.2 Hz, 1H), 7.25 (s, 1H), 7.15 (t, J=7.4 Hz, 1H), 7.06 (d, J=8.6 Hz, 1H), 7.00 (s, 1H), 6.61 (s, 1H), 6.45 (d, J=8.2 Hz, 1H), 5.30 (s, 2H), 4.60-4.52 (m, 1H), 3.74 (s, 3H), 3.71 (s, 2H), 3.56 (s, 2H), 3.46 (s, 2H), 3.29 (s, 5H), 3.21 (s, 2H), 3.18 (s, 3H), 3.10 (s, 3H), 2.69-2.54 (m, 10H), 2.43-2.31 (m, 2H), 2.08-1.92 (m, 2H), 1.84 (d, J=11.5 Hz, 2H), 1.55 (d, J=10.7 Hz, 2H); [M+H]⁺=969.4.

### Example 131: N-(2-((5-chloro-2-((4-(4-((5-(4-(2,6-dioxopiperidin-3-yl)phenyl)pentyl)amino)piperidin-1-yl)-2-methoxyphenyl)amino)pyrimidin-4-yl)amino)phenyl)-N-methylmethanesulfonamide

### Step 1: 5-(4-bromophenyl)pent-4-yn-1-ol

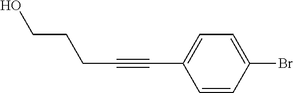 [SMILES: OCCCC#Cc1ccc(Br)cc1]

A mixture of 1-bromo-4-iodobenzene (3.0 g, 10.6 mmol), pent-4-yn-1-ol (0.98 g, 11.7 mmol), CuI (204 mg, 1.06 mmol), Pd(PPh₃)₂Cl₂ (373 mg, 0.53 mmol) and piperidine (1.8 g, 21.2 mmol) in toluene (30 mL) was stirred in a flask at 40° C. overnight under N₂. The mixture was evaporated in vacuum to afford the crude product, which was further purified with silica gel column chromatography (PE: EA=100: 0˜ 2: 1 gradient elution) to give the product (1.5 g, 59%). [M+H]⁺=239.0.

### Step 2: 5-(4-(4,4,5,5-tetramethyl-1,3,2-dioxaborolan-2-yl)phenyl)pent-4-yn-1-ol

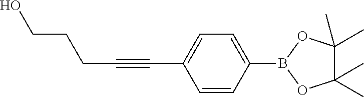 [SMILES: CC1(C)OB(c2ccc(C#CCCCO)cc2)OC1(C)C]

A mixture of 5-(4-bromophenyl)pent-4-yn-1-ol (1.2 g, 5.0 mmol), 4,4,4′,4′,5,5,5′,5′-octamethyl-2,2′-bi(1,3,2-dioxaborolane) (1.5 g, 6.0 mmol), Pd(dppf)Cl₂ (367 mg, 0.5 mmol) and KOAc (1.5 g, 15.0 mmol) in 1,4-dioxane (30 mL) was stirred in a flask at 80° C. for 1 h. The mixture was evaporated in vacuum to afford the crude product, which was further purified with silica gel column chromatography (PE: EA=100: 0˜ 4: 1 gradient elution) to give the title product (1.4 g, 97%). [M+H]⁺=287.2.

### Step 3: 5-(4-(2,6-bis(benzyloxy)pyridin-3-yl)phenyl)pent-4-yn-1-ol

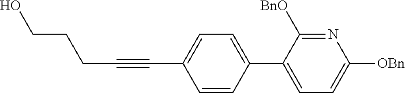 [SMILES: OCCCC#Cc1ccc(-c2ccc(OCc3ccccc3)nc2OCc2ccccc2)cc1]

A mixture of 5-(4-(4,4,5,5-tetramethyl-1,3,2-dioxaborolan-2-yl)phenyl)pent-4-yn-1-ol (1.4 g, 4.9 mmol), 2,6-bis(benzyloxy)-3-bromopyridine (1.8 g, 4.9 mmol), Pd(dppf)Cl₂ (366 mg, 0.5 mmol) and Cs₂CO₃ (2.5 g, 7.5 mmol) in 1,4-dioxane (50 mL) and H₂O (10 mL) was stirred in a flask at 80° C. for 2 h. The mixture was evaporated in vacuum to afford the crude product, which was further purified with silica gel column chromatography (PE: EA=100: 0˜ 2: 1 gradient elution) to give the product (2.0 g, 91%). [M+H]⁺=450.2.

### Step 4: 3-(4-(5-hydroxypentyl)phenyl)piperidine-2,6-dione

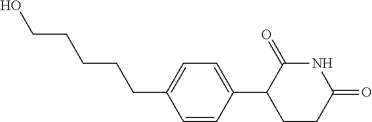 [SMILES: O=C1CCC(c2ccc(CCCCCO)cc2)C(=O)N1]

Under N₂, to a solution of 5-(4-(2,6-bis(benzyloxy)pyridin-3-yl)phenyl)pent-4-yn-1-ol (2.0 g, 4.4 mmol) in MeOH (100 mL) was added 10% Pd/C (400 mg) at 25° C. And then the mixture was exchanged with H₂ two times and stirred under H₂ atmosphere at 25° C. for 48 h. Reaction was monitored by HPLC. The mixture was filtered through a pad of Celite and washed with MeOH (50 mL). The filtrate was concentrated under vacuum to obtain the product (1.2 g, 97%). [M+H]⁺=276.2.

### Step 5: 5-(4-(2,6-dioxopiperidin-3-yl)phenyl)pentanal

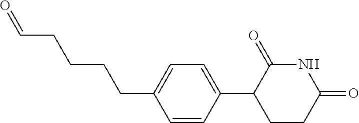 [SMILES: O=CCCCCc1ccc(C2CCC(=O)NC2=O)cc1]

To a solution of 3-(4-(5-hydroxypentyl)phenyl)piperidine-2,6-dione (100 mg, 0.36 mmol) in DMSO (3.0 mL) was added IBX (203 mg, 0.72 mmol) in portions at 25° C. The mixture was stirred at 25° C. for 4 h. Reaction was monitored by HPLC. Water (20.0 mL) was added and the resulting solution was extracted with EtOAc (2×30.0 mL). The combined organic layer was washed with brine (3×20.0 mL), dried over Na₂SO₄ and concentrated under vacuum to afford the title product (80 mg, 81%). [M+H]⁺=274.2.

### Step 6: N-(2-((5-chloro-2-((4-(4-((5-(4-(2,6-dioxopiperidin-3-yl)phenyl)pentyl)amino)piperidin-1-yl)-2-methoxyphenyl)amino)pyrimidin-4-yl)amino)phenyl)-N-methylmethanesulfonamide

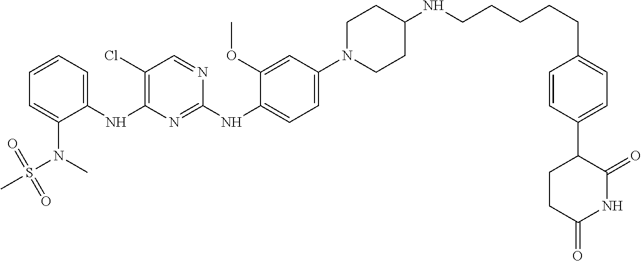 [SMILES: COc1cc(N2CCC(NCCCCCc3ccc(C4CCC(=O)NC4=O)cc3)CC2)ccc1Nc1ncc(Cl)c(Nc2ccccc2N(C)S(C)(=O)=O)n1]

A mixture of N-(2-((2-((4-(4-aminopiperidin-1-yl)-2-methoxyphenyl)amino)-5-chloropyrimidin-4-yl)amino)phenyl)-N-methylmethanesulfonamide (50 mg, 0.09 mmol), 5-(4-(2,6-dioxopiperidin-3-yl)phenyl)pentanal (24 mg, 0.09 mmol) and NaOAc (22 mg, 0.27 mmol) in DCM (5 mL) and MeOH (1 mL) was stirred in a flask at room temperature for 1 hour. The mixture was added NaBH₃CN (16 mg, 0.27 mmol) and stirred in a flask at room temperature for 2 h. The reaction was quenched with water (10 mL) and extracted with DCM (2×20 mL). The combined organic layers were dried over anhydrous Na₂SO₄, and evaporated in vacuum to afford the crude product, which was further purified with pre-HPLC to give the product (12 mg, 16%). ¹H NMR (400 MHz, DMSO) δ_{H} 10.80 (s, 1H), 8.36-8.17 (m, 3H), 8.16-7.95 (m, 2H), 7.56 (d, J=5.8 Hz, 1H), 7.34 (s, 1H), 7.23 (s, 1H), 7.17-7.01 (m, 5H), 6.60 (s, 1H), 6.42 (s, 1H), 3.81-3.57 (m, 6H), 3.15 (d, J=6.0 Hz, 3H), 3.07 (d, J=6.0 Hz, 3H), 2.75-2.51 (m, 8H), 2.44-2.37 (m, 1H), 2.20-1.85 (m, 4H), 1.61-1.24 (m, 8H); [M+H]⁺=789.4.

### Example 132: N-(2-((5-chloro-2-((4-(4-((7-(4-(2,6-dioxopiperidin-3-yl)phenyl)heptyl)amino)piperidin-1-yl)-2-methoxyphenyl)amino)pyrimidin-4-yl)amino)phenyl)-N-methylmethanesulfonamide

### Step 1: tert-butyl (1-(3-methoxy-4-nitrophenyl)piperidin-4-yl)carbamate

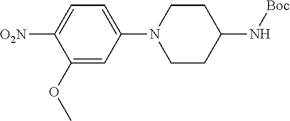 [SMILES: COc1cc(N2CCC(NC(=O)OC(C)(C)C)CC2)ccc1[N+](=O)[O-]]

A mixture of 4-fluoro-2-methoxy-1-nitrobenzene (1.0 g, 5.8 mmol), tert-butyl piperidin-4-ylcarbamate (1.23 g, 6.1 mmol) and K₂CO₃ (1.6 g, 11.6 mmol) in DMF (15 mL) was stirred in a flask at 60° C. overnight. The reaction was cooled to room temperature, the mixture was poured into water (50 mL) and stirred for 10 mins. The solid was filtered and washed with water (20 mL×2), dried to give the product (2.0 g, 98%). [M+H]⁺=352.2.

### Step 2: tert-butyl (1-(4-amino-3-methoxyphenyl)piperidin-4-yl)carbamate

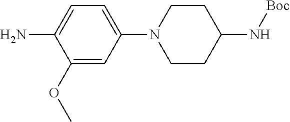 [SMILES: COc1cc(N2CCC(NC(=O)OC(C)(C)C)CC2)ccc1N]

Under N₂, to a solution of tert-butyl (1-(3-methoxy-4-nitrophenyl)piperidin-4-yl)carbamate (2.0 g, 5.7 mmol) in MeOH (50 mL) was added 10% Pd/C (400 mg) at 25° C. And then the mixture was exchanged with H₂ two times and stirred under H₂ atmosphere at 25° C. for 2 h. The mixture was filtered through a pad of Celite and washed with MeOH (50 mL). The filtrate was concentrated under vacuum to obtain the product (1.8 g, 98%). [M+H]⁺=322.2.

### Step 3: tert-butyl(1-(4-((5-chloro-4-((2-(N-methylmethylsulfonamido)phenyl)amino)pyrimidin-2-yl)amino)-3-methoxyphenyl)piperidin-4-yl)carbamate

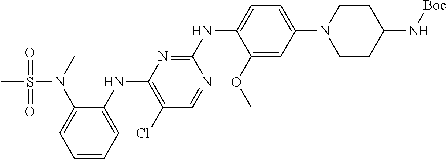 [SMILES: COc1cc(N2CCC(NC(=O)OC(C)(C)C)CC2)ccc1Nc1ncc(Cl)c(Nc2ccccc2N(C)S(C)(=O)=O)n1]

A mixture of N-(2-((2,5-dichloropyrimidin-4-yl)amino)phenyl)-N-methylmethanesulfonamide (410 mg, 1.2 mmol), tert-butyl (1-(4-amino-3-methoxyphenyl)piperidin-4-yl)carbamate (381 mg, 1.2 mmol), Pd₂(dba)₃ (108 mg, 0.12 mmol), BINAP (147 mg, 0.24 mmol) and K₃PO₄ (753 mg, 3.75 mmol) in toluene (20 mL) was stirred in a flask at 100° C. overnight under N₂. The mixture was evaporated in vacuum to afford the crude product, which was further purified with silica gel column chromatography (DCM: MeOH=100: 0˜ 10: 1 gradient elution) to give the product (450 mg, 60%). [M+H]⁺=632.2.

### Step 4: N-(2-((2-((4-(4-aminopiperidin-1-yl)-2-methoxyphenyl)amino)-5-chloropyrimidin-4-yl)amino)phenyl)-N-methylmethanesulfonamide

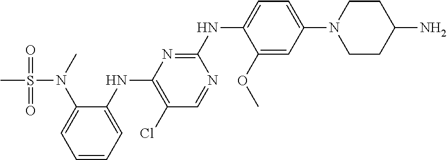 [SMILES: COc1cc(N2CCC(N)CC2)ccc1Nc1ncc(Cl)c(Nc2ccccc2N(C)S(C)(=O)=O)n1]

A solution of tert-butyl (1-(4-((5-chloro-4-((2-(N-methylmethylsulfonamido)phenyl)amino)pyrimidin-2-yl)amino)-3-methoxyphenyl)piperidin-4-yl)carbamate (450 mg, 0.71 mmol) in HCl/1,4-dioxane (15 mL) was stirred in a flask at room temperature for 2 h. The mixture was evaporated in vacuum to afford the crude product (375 mg, 98%), which was used for next step without further purification. [M+H]⁺=532.2.

### Step 5: 7-(4-bromophenyl)hept-6-yn-1-ol

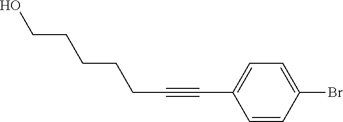 [SMILES: OCCCCCC#Cc1ccc(Br)cc1]

A mixture of 1-bromo-4-iodobenzene (3.0 g, 10.6 mmol), hept-6-yn-1-ol (1.3 g, 11.7 mmol), CuI (204 mg, 1.06 mmol), Pd(PPh₃)₂Cl₂ (373 mg, 0.53 mmol) and piperidine (1.8 g, 21.2 mmol) in toluene (20 mL) was stirred in a flask at 40° C. overnight under N₂. The mixture was evaporated in vacuum to afford the crude product, which was further purified with silica gel column chromatography (PE: EA=100: 0˜ 2: 1 gradient elution) to give the product (2.4 g, 85%). [M+H]⁺=267.1.

### Step 6: 7-(4-(4,4,5,5-tetramethyl-1,3,2-dioxaborolan-2-yl)phenyl)hept-6-yn-1-ol

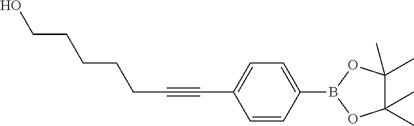 [SMILES: CC1(C)OB(c2ccc(C#CCCCCCO)cc2)OC1(C)C]

A mixture of 7-(4-bromophenyl)hept-6-yn-1-ol (2.4 g, 9.0 mmol), 4,4,4′,4′,5,5,5′,5′-octamethyl-2,2′-bi(1,3,2-dioxaborolane) (2.7 g, 10.8 mmol), Pd(dppf)Cl₂ (650 mg, 0.9 mmol) and KOAc (2.6 g, 27.0 mmol) in 1,4-dioxane (50 mL) was stirred in a flask at 80° C. for 1 h. The mixture was evaporated in vacuum to afford the crude product, which was further purified with silica gel column chromatography (PE: EA=100: 0˜ 4: 1 gradient elution) to give the title product (2.8 g, 99%). [M+H]⁺=315.2.

### Step 7: 7-(4-(2,6-bis(benzyloxy)pyridin-3-yl)phenyl)hept-6-yn-1-ol

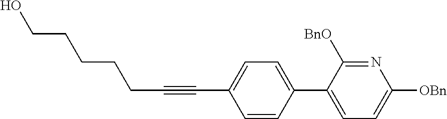 [SMILES: OCCCCCC#Cc1ccc(-c2ccc(OCc3ccccc3)nc2OCc2ccccc2)cc1]

A mixture of 7-(4-(4,4,5,5-tetramethyl-1,3,2-dioxaborolan-2-yl)phenyl)hept-6-yn-1-ol (2.8 g, 8.9 mmol), 2,6-bis(benzyloxy)-3-bromopyridine (3.3 g, 8.9 mmol), Pd(dppf)Cl₂ (658 mg, 0.89 mmol) and Cs₂CO₃ (4.4 g, 13.5 mmol) in 1,4-dioxane (75 mL) and H₂O (15 mL) was stirred in a flask at 80° C. for 2 h. The mixture was evaporated in vacuum to afford the crude product, which was further purified with silica gel column chromatography (PE: EA=100: 0˜ 2: 1 gradient elution) to give the product (3.2 g, 75%). [M+H]⁺=478.3.

### Step 8: 3-(4-(7-hydroxyheptyl)phenyl)piperidine-2,6-dione

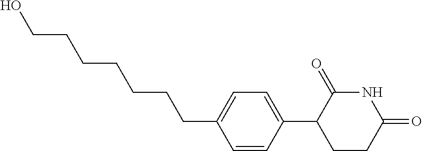 [SMILES: O=C1CCC(c2ccc(CCCCCCCO)cc2)C(=O)N1]

Under N₂, to a solution of 7-(4-(2,6-bis(benzyloxy)pyridin-3-yl)phenyl)hept-6-yn-1-ol (3.2 g, 6.7 mmol) in MeOH (150 mL) was added 10% Pd/C (500 mg) at 25° C. And then the mixture was exchanged with H₂ two times and stirred under H₂ atmosphere at 25° C. for 48 h. Reaction was monitored by HPLC. The mixture was filtered through a pad of Celite and washed with MeOH (100 mL). The filtrate was concentrated under vacuum to obtain the product (2.0 g, 98%). [M+H]⁺=304.2.

### Step 9: 7-(4-(2,6-dioxopiperidin-3-yl)phenyl)heptanal

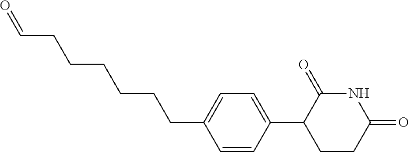 [SMILES: O=CCCCCCCc1ccc(C2CCC(=O)NC2=O)cc1]

To a solution of 3-(4-(7-hydroxyheptyl)phenyl)piperidine-2,6-dione (52 mg, 0.17 mmol) in DMSO (2.0 mL) was added IBX (96 mg, 0.34 mmol) in portions at 25° C. The mixture was stirred at 25° C. for 4 h.

Reaction was monitored by HPLC. Water (20.0 mL) was added to the reaction at 25° C. The resulting solution was extracted with 2×30.0 mL of EtOAc. The combined organic layer was washed with brine (3×20.0 mL), dried over Na₂SO₄ and concentrated under vacuum to afford the title product (50 mg, 97%). [M+H]⁺=302.2.

### Step 10: N-(2-((5-chloro-2-((4-(4-((7-(4-(2,6-dioxopiperidin-3-yl)phenyl)heptyl)amino)piperidin-1-yl)-2-methoxyphenyl)amino)pyrimidin-4-yl)amino)phenyl)-N-methylmethanesulfonamide

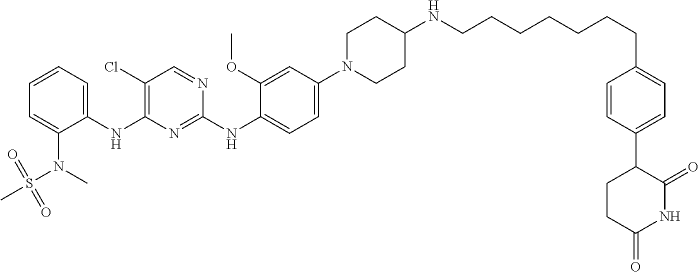 [SMILES: COc1cc(N2CCC(NCCCCCCCc3ccc(C4CCC(=O)NC4=O)cc3)CC2)ccc1Nc1ncc(Cl)c(Nc2ccccc2N(C)S(C)(=O)=O)n1]

A mixture of N-(2-((2-((4-(4-aminopiperidin-1-yl)-2-methoxyphenyl)amino)-5-chloropyrimidin-4-yl)amino)phenyl)-N-methylmethanesulfonamide (47 mg, 0.09 mmol), 7-(4-(2,6-dioxopiperidin-3-yl)phenyl)heptanal (25 mg, 0.08 mmol) and NaOAc (20 mg, 0.24 mmol) in DCM (5 mL) and MeOH (1 mL) was stirred in a flask at room temperature for 1 hour. The mixture was added NaBH₃CN (15 mg, 0.24 mmol) and stirred in a flask at room temperature for 2 h. The reaction was quenched with water (10 mL) and extracted with DCM (20 mL×2). The combined organic layers were dried over anhydrous Na₂SO₄, and evaporated in vacuum to afford the crude product, which was further purified with pre-HPLC to give the product (24 mg, 35%). ¹H NMR (400 MHz, DMSO) δ_{H} 10.84 (s, 1H), 8.36 (s, 1H), 8.29 (s, 2H), 8.12 (d, J=7.2 Hz, 2H), 7.60 (d, J=7.6 Hz, 1H), 7.39 (d, J=8.1 Hz, 1H), 7.27 (s, 1H), 7.14 (dd, J=14.7, 7.7 Hz, 5H), 6.64 (s, 1H), 6.47 (d, J=8.3 Hz, 1H), 3.85-3.64 (m, 6H), 3.19 (s, 3H), 3.11 (s, 3H), 2.85-2.53 (m, 8H), 2.46 (s, 1H), 2.18 (d, J=11.3 Hz, 1H), 2.09-1.91 (m, 3H), 1.54 (d, J=31.4 Hz, 6H), 1.32 (s, 6H); [M+H]⁺=817.4.

### Example 133: N-(2-((5-chloro-2-((4-(4-(4-(2,6-dioxopiperidin-3-yl)phenethyl) piperazin-1-yl)-2-methoxyphenyl)amino)pyrimidin-4-yl)amino)phenyl)-N-methylmethanesulfonamide

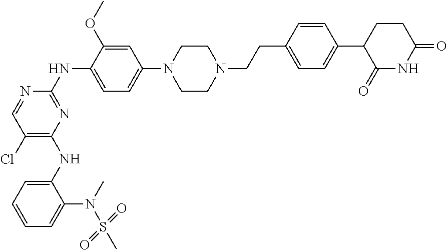 [SMILES: COc1cc(N2CCN(CCc3ccc(C4CCC(=O)NC4=O)cc3)CC2)ccc1Nc1ncc(Cl)c(Nc2ccccc2N(C)S(C)(=O)=O)n1]

The titled compound was synthesized in the procedures similar to Example 119. ¹H NMR (400 MHz, DMSO) δ_{H} 10.83 (s, 1H), 8.28 (s, 2H), 8.12 (d, J=13.0 Hz, 2H), 7.59 (d, J=7.6 Hz, 1H), 7.39 (d, J=7.6 Hz, 1H), 7.28-7.08 (m, 6H), 6.64 (s, 1H), 6.47 (d, J=8.2 Hz, 1H), 3.85-3.74 (m, 4H), 3.18 (s, 7H), 3.10 (s, 3H), 2.79 (s, 2H), 2.70-2.58 (m, 7H), 2.47-2.43 (m, 1H), 2.17 (s, 1H), 2.04 (s, 1H); [M+H]⁺=733.3.

### Example 134: N-(2-((5-chloro-2-((4-(6-(4-(2,6-dioxopiperidin-3-yl)phenethyl)-2,6-diazaspiro[3.3]heptan-2-yl)-3-fluorophenyl)amino)pyrimidin-4-yl)amino)phenyl)-N-methylmethanesulfonamide

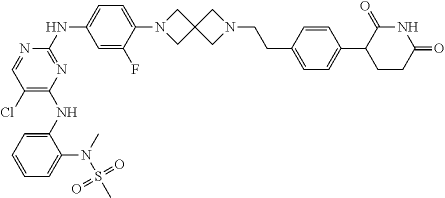 [SMILES: CN(c1ccccc1Nc1nc(Nc2ccc(N3CC4(CN(CCc5ccc(C6CCC(=O)NC6=O)cc5)C4)C3)c(F)c2)ncc1Cl)S(C)(=O)=O]

The titled compound was synthesized in the procedures similar to Example 119. ¹H NMR (400 MHz, DMSO) δ 10.83 (s, 1H), 9.34 (s, 1H), 8.36 (s, 1H), 8.30 (s, 1H), 8.19 (s, 2H), 7.64 (d, J=7.6 Hz, 1H), 7.49 (d, J=15.3 Hz, 1H), 7.39 (t, J=7.3 Hz, 1H), 7.26 (t, J=7.6 Hz, 1H), 7.14-7.08 (m, 5H), 6.45 (t, J=9.3 Hz, 1H), 3.88 (s, 4H), 3.81 (d, J=9.0 Hz, 1H), 3.32 (s, 4H), 3.19 (s, 3H), 3.10 (s, 3H), 2.71-2.52 (m, 6H), 2.24-2.12 (m, 1H), 2.02 (dd, J=14.0, 7.8 Hz, 1H); [M+H]⁺=733.2.

### Example 135: N-(2-((5-chloro-2-((4-(6-(3-(4-((2,6-dioxopiperidin-3-yl)amino)phenoxy)propyl)-2,6-diazaspiro[3.3]heptan-2-yl)-3-fluorophenyl)amino)pyrimidin-4-yl)amino)phenyl)-N-methylmethanesulfonamide

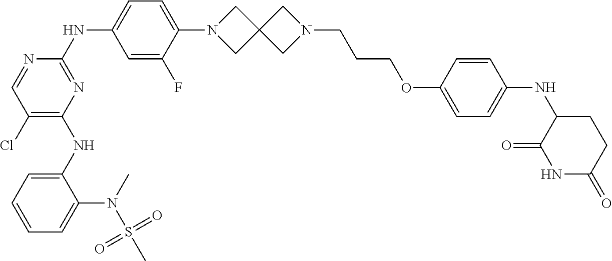 [SMILES: CN(c1ccccc1Nc1nc(Nc2ccc(N3CC4(CN(CCCOc5ccc(NC6CCC(=O)NC6=O)cc5)C4)C3)c(F)c2)ncc1Cl)S(C)(=O)=O]

The titled compound was synthesized in the procedures similar to Example 47. ¹H NMR (400 MHz, DMSO) δ 10.77 (s, 1H), 9.34 (s, 1H), 8.36 (s, 1H), 8.29 (s, 1H), 8.19 (s, 2H), 7.63 (d, J=7.8 Hz, 1H), 7.48 (d, J=16.0 Hz, 1H), 7.39 (t, J=7.6 Hz, 1H), 7.25 (t, J=7.7 Hz, 1H), 7.14 (d, J=8.0 Hz, 1H), 6.71 (d, J=8.5 Hz, 2H), 6.62 (d, J=8.7 Hz, 2H), 6.45 (t, J=9.4 Hz, 1H), 5.43 (s, 1H), 4.20 (s, 1H), 3.87 (d, J=16.3 Hz, 7H), 3.19 (s, 3H), 3.10 (s, 3H), 2.71 (d, J=11.7 Hz, 1H), 2.57-2.49 (m, 5H), 2.09 (s, 1H), 1.84 (d, J=8.6 Hz, 1H), 1.67 (s, 2H); [M+H]⁺=778.3.

### Example 136: N-(2-((5-chloro-2-((4-(6-(4-((2,6-dioxopiperidin-3-yl)amino)phenethyl)-2,6-diazaspiro[3.3]heptan-2-yl)-3-fluorophenyl)amino)pyrimidin-4-yl)amino)phenyl)-N-methylmethanesulfonamide

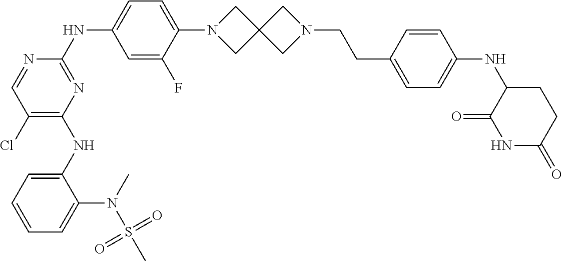 [SMILES: CN(c1ccccc1Nc1nc(Nc2ccc(N3CC4(CN(CCc5ccc(NC6CCC(=O)NC6=O)cc5)C4)C3)c(F)c2)ncc1Cl)S(C)(=O)=O]

The titled compound was synthesized in the procedures similar to Example 48. ¹H NMR (400 MHz, DMSO)¹H NMR (400 MHz, DMSO) δ 10.78 (s, 1H), 9.33 (s, 1H), 8.36 (s, 1H), 8.30 (s, 1H), 8.23 (s, 1H), 8.18 (s, 1H), 7.63 (d, J=7.8 Hz, 1H), 7.48 (d, J=15.2 Hz, 1H), 7.38 (d, J=7.4 Hz, 1H), 7.26 (d, J=7.5 Hz, 1H), 7.15 (s, 1H), 6.91 (d, J=7.8 Hz, 2H), 6.59 (d, J=7.4 Hz, 2H), 6.44 (t, J=9.2 Hz, 1H), 5.66 (d, J=7.6 Hz, 1H), 4.27 (s, 1H), 3.87 (s, 4H), 3.29 (s, 5H), 3.19 (s, 3H), 3.10 (s, 3H), 2.72 (d, J=11.5 Hz, 2H), 2.40 (s, 2H), 2.08 (s, 1H), 1.87 (s, 1H); [M+H]⁺=748.2.

### Example 137: N-(2-((5-chloro-2-((4-(6-(1-((1-(2-(2,6-dioxopiperidin-3-yl)-1,3-dioxoisoindolin-5-yl)piperidin-4-yl)methyl)piperidine-4-carbonyl)-2,6-diazaspiro[3.3]heptan-2-yl)-3-fluorophenyl)amino)pyrimidin-4-yl)amino)phenyl)-N-methylmethanesulfonamide

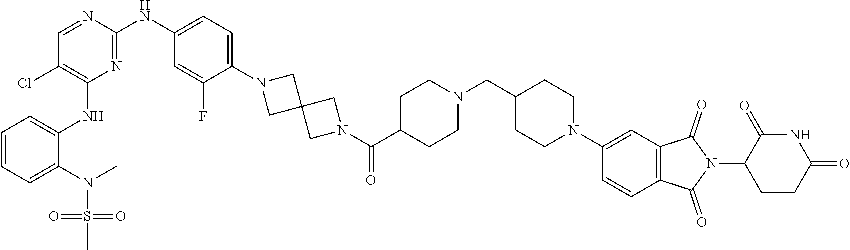 [SMILES: CN(c1ccccc1Nc1nc(Nc2ccc(N3CC4(CN(C(=O)C5CCN(CC6CCN(c7ccc8c(c7)C(=O)N(C7CCC(=O)NC7=O)C8=O)CC6)CC5)C4)C3)c(F)c2)ncc1Cl)S(C)(=O)=O]

The titled compound was synthesized in the procedures similar to Example 63. ¹H NMR (400 MHz, DMSO) δ 11.09 (s, 1H), 9.36 (s, 1H), 8.37 (s, 1H), 8.31 (s, 1H), 8.19 (s, 1H), 7.65 (t, J=8.2 Hz, 2H), 7.53 (s, 1H), 7.40 (d, J=7.9 Hz, 1H), 7.33 (s, 1H), 7.26 (t, J=8.0 Hz, 2H), 7.15 (d, J=7.8 Hz, 1H), 6.47 (t, J=9.4 Hz, 1H), 5.07 (dd, J=12.8, 5.2 Hz, 1H), 4.34 (s, 2H), 4.08-3.94 (m, 8H), 3.19 (s, 3H), 3.10 (s, 3H), 3.02-2.82 (m, 4H), 2.57 (d, J=24.7 Hz, 4H), 2.50-2.23 (m, 4H), 1.99-1.76 (m, 2H), 1.75-1.62 (m, 6H), 1.17 (d, J=10.2 Hz, 2H); [M+H]⁺=982.4.

### Example 138: N-(2-((5-chloro-2-((4-(4-(4-(4-(2,6-dioxopiperidin-3-yl)phenethyl)piperazin-1-yl)piperidin-1-yl)-2-methoxyphenyl)amino)pyrimidin-4-yl)amino)phenyl)methanesulfonamide

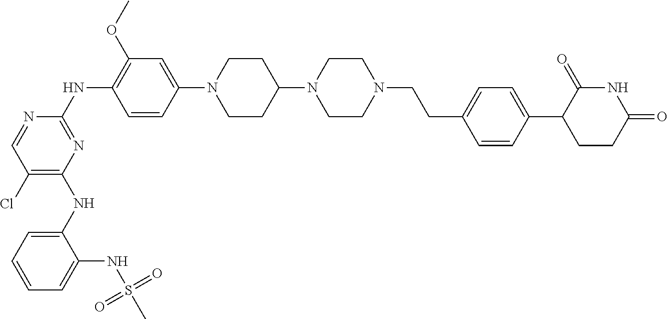 [SMILES: COc1cc(N2CCC(N3CCN(CCc4ccc(C5CCC(=O)NC5=O)cc4)CC3)CC2)ccc1Nc1ncc(Cl)c(Nc2ccccc2NS(C)(=O)=O)n1]

The titled compound was synthesized in the procedures similar to Example 119. ¹H NMR (400 MHz, DMSO) δ 10.83 (s, 1H), 8.51 (s, 1H), 8.08 (s, 1H), 8.00 (d, J=6.1 Hz, 1H), 7.90 (s, 1H), 7.39 (s, 1H), 7.34 (d, J=6.7 Hz, 1H), 7.15-7.10 (m, 7H), 6.59 (s, 1H), 6.37 (d, J=7.9 Hz, 1H), 3.81 (d, J=7.5 Hz, 1H), 3.75 (s, 3H), 3.69 (d, J=10.5 Hz, 2H), 2.93 (s, 3H), 2.66-2.53 (m, 7H), 2.33 (s, 6H), 2.17 (d, J=9.8 Hz, 2H), 2.03 (s, 2H), 1.86 (d, J=10.4 Hz, 3H), 1.52 (d, J=11.0 Hz, 3H); [M+H]⁺=802.4.

### Example 139: N-(2-((5-chloro-2-((4-(4-(4-(2-(1-(4-(2,4-dioxotetrahydropyrimidin-1(2H)-yl)phenyl)piperidin-4-yl)ethyl)piperazin-1-yl)piperidin-1-yl)-2-methoxy phenyl)amino)pyrimidin-4-yl)amino)phenyl) methanesulfonamide

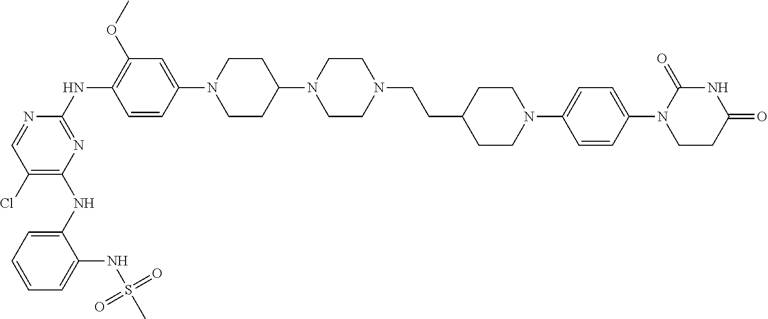 [SMILES: COc1cc(N2CCC(N3CCN(CCC4CCN(c5ccc(N6CCC(=O)NC6=O)cc5)CC4)CC3)CC2)ccc1Nc1ncc(Cl)c(Nc2ccccc2NS(C)(=O)=O)n1]

The titled compound was synthesized in the procedures similar to Example 79. ¹H NMR (400 MHz, DMSO) δ 10.27 (s, 1H), 8.47 (s, 1H), 8.08 (s, 1H), 7.99 (s, 1H), 7.90 (s, 1H), 7.38-7.30 (m, 3H), 7.28-7.01 (m, 5H), 6.92 (d, J=7.9 Hz, 2H), 6.59 (s, 1H), 6.37 (d, J=7.9 Hz, 1H), 3.75 (s, 3H), 3.69 (s, 7H), 2.95 (s, 3H), 2.63-2.50 (m, 15H), 1.88 (d, J=10.0 Hz, 2H), 1.74 (d, J=11.4 Hz, 2H), 1.54 (d, J=10.6 Hz, 2H), 1.44 (s, 3H), 1.25 (d, J=9.0 Hz, 2H); [M+H]⁺=886.5.

### Example 140: N-(2-((5-chloro-2-((4-(4-(4-(4-((2,6-dioxopiperidin-3-yl)amino)phenethyl)piperazin-1-yl)piperidin-1-yl)-2-methoxyphenyl)amino)pyrimidin-4-yl)amino)phenyl)methanesulfonamide

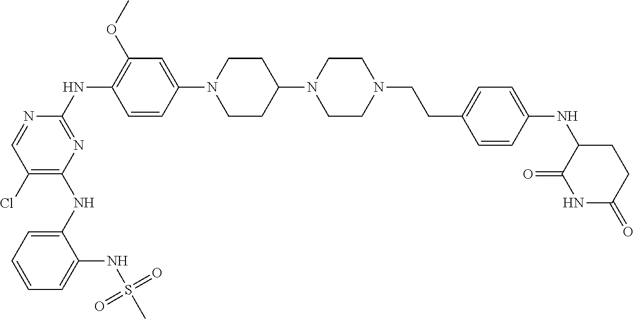 [SMILES: COc1cc(N2CCC(N3CCN(CCc4ccc(NC5CCC(=O)NC5=O)cc4)CC3)CC2)ccc1Nc1ncc(Cl)c(Nc2ccccc2NS(C)(=O)=O)n1]

The titled compound was synthesized in the procedures similar to Example 48. ¹H NMR (400 MHz, DMSO) δ 10.78 (s, 1H), 8.63 (s, 1H), 8.22 (s, 1H), 8.06 (s, 2H), 7.89 (s, 1H), 7.42 (d, J=8.1 Hz, 1H), 7.32 (s, 1H), 7.09 (s, 2H), 6.93 (d, J=7.6 Hz, 2H), 6.60 (s, 3H), 6.39 (d, J=8.5 Hz, 1H), 5.65 (d, J=6.6 Hz, 1H), 4.27 (s, 1H), 3.75 (s, 3H), 3.69 (d, J=10.2 Hz, 2H), 2.89 (s, 3H), 2.66 (d, J=11.0 Hz, 8H), 2.33 (s, 6H), 2.09 (s, 2H), 1.86 (d, J=10.4 Hz, 4H), 1.52 (d, J=10.0 Hz, 3H); [M+H]⁺=817.4.

### Example 141: N-(2-((5-chloro-2-((4-(4-(4-(5-(4-(2-(2,6-dioxopiperidin-3-yl)-1,3-dioxoisoindolin-5-yl)piperazin-1-yl)pentanoyl)piperazin-1-yl)piperidin-1-yl)-2-methoxyphenyl)amino)pyrimidin-4-yl)amino)phenyl)methanesulfonamide

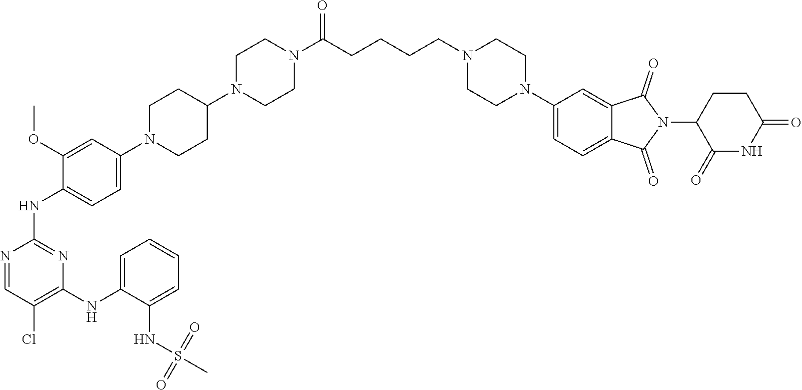 [SMILES: COc1cc(N2CCC(N3CCN(C(=O)CCCCN4CCN(c5ccc6c(c5)C(=O)N(C5CCC(=O)NC5=O)C6=O)CC4)CC3)CC2)ccc1Nc1ncc(Cl)c(Nc2ccccc2NS(C)(=O)=O)n1]

The titled compound was synthesized in the procedures similar to Example 62. ¹H NMR (400 MHz, DMSO) δ 11.06 (s, 1H), 8.75 (s, 1H), 8.22 (s, 2H), 8.02 (s, 1H), 7.86 (s, 1H), 7.65 (d, J=7.7 Hz, 1H), 7.41 (s, 1H), 7.32 (s, 1H), 7.24 (s, 2H), 6.98 (s, 2H), 6.57 (s, 1H), 6.37 (s, 1H), 5.03 (s, 1H), 3.73 (s, 3H), 3.67 (d, J=11.4 Hz, 2H), 3.41 (s, 9H), 2.81 (s, 3H), 2.71-2.53 (m, 6H), 2.30 (s, 10H), 1.99 (s, 2H), 1.79 (s, 2H), 1.49 (s, 6H); [M+H]⁺=1011.6.

### Example 142: N-(2-((5-chloro-2-((4-(4-(4-(1-((1-(2-(2,6-dioxopiperidin-3-yl)-1,3-dioxoisoindolin-5-yl)piperidin-4-yl)methyl)piperidine-4-carbonyl)piperazin-1-yl)piperidin-1-yl)-2-methoxyphenyl)amino)pyrimidin-4-yl)amino)phenyl)methanesulfonamide

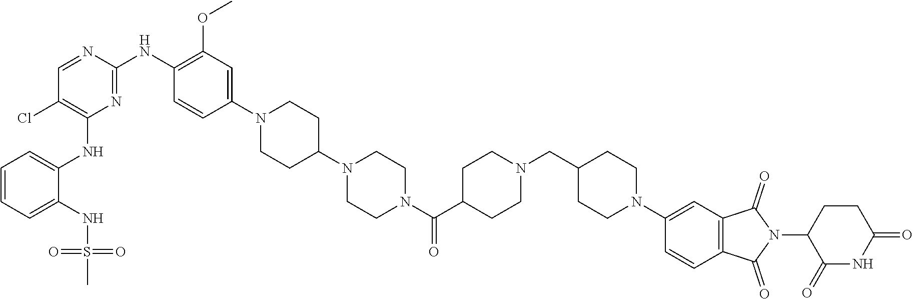 [SMILES: COc1cc(N2CCC(N3CCN(C(=O)C4CCN(CC5CCN(c6ccc7c(c6)C(=O)N(C6CCC(=O)NC6=O)C7=O)CC5)CC4)CC3)CC2)ccc1Nc1ncc(Cl)c(Nc2ccccc2NS(C)(=O)=O)n1]

The titled compound was synthesized in the procedures similar to Example 63. ¹H NMR (400 MHz, DMSO) δ 11.08 (s, 1H), 8.53 (s, 1H), 8.17 (s, 1H), 8.07 (s, 1H), 8.00 (s, 1H), 7.89 (s, 1H), 7.65 (d, J=8.7 Hz, 1H), 7.41 (d, J=9.5 Hz, 1H), 7.32 (d, J=8.6 Hz, 2H), 7.24 (s, 1H), 7.15 (s, 2H), 6.59 (s, 1H), 6.39 (s, 1H), 5.05 (s, 1H), 4.04 (d, J=11.3 Hz, 2H), 3.75 (s, 3H), 3.70 (d, J=11.9 Hz, 2H), 3.46 (s, 4H), 2.91-2.80 (m, 9H), 2.70-2.57 (m, 4H), 2.45-2.33 (m, 3H), 2.13 (s, 2H), 1.93 (s, 4H), 1.78 (d, J=12.9 Hz, 6H), 1.58 (s, 6H), 1.14 (s, 2H); [M+H]⁺=1051.7.

### Example 143: N-(2-((5-chloro-2-((4-(4-(4-(3-(4-((2,6-dioxopiperidin-3-yl)amino)phenoxy)propyl) piperazin-1-yl)piperidin-1-yl)-2-methoxyphenyl)amino)pyrimidin-4-yl)amino)phenyl) methanesulfonamide

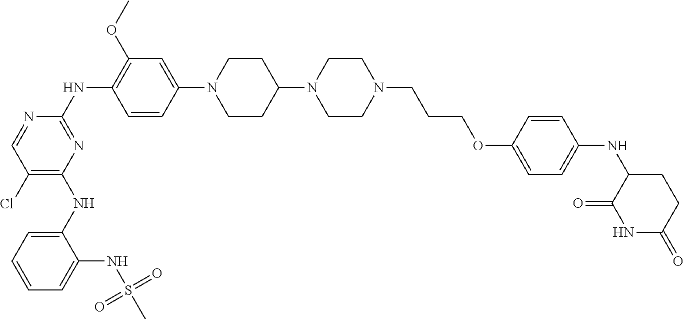 [SMILES: COc1cc(N2CCC(N3CCN(CCCOc4ccc(NC5CCC(=O)NC5=O)cc4)CC3)CC2)ccc1Nc1ncc(Cl)c(Nc2ccccc2NS(C)(=O)=O)n1]

The titled compound was synthesized in the procedures similar to Example 47. ¹H NMR (400 MHz, DMSO) δ 10.78 (s, 1H), 8.57 (s, 1H), 8.19 (s, 1H), 8.08 (s, 1H), 8.02 (s, 1H), 7.90 (s, 1H), 7.41 (s, 1H), 7.33 (s, 1H), 7.14 (s, 2H), 6.71 (s, 2H), 6.63 (s, 3H), 6.39 (s, 1H), 5.43 (s, 1H), 4.21 (s, 1H), 3.88 (s, 2H), 3.76 (s, 3H), 3.68 (s, 2H), 2.92 (s, 3H), 2.63 (d, J=15.6 Hz, 8H), 2.41 (s, 7H), 2.10 (s, 1H), 1.84 (s, 5H), 1.54 (s, 2H); [M+H]⁺=847.4.

### Example 144: 3-(4-(4-(4-((5-chloro-4-((2-(dimethylphosphoryl)phenyl)amino)pyrimidin-2-yl)amino)-3-methoxybenzyl)piperidin-1-yl)phenyl)piperidine-2,6-dione

### Step 1: 2,6-bis(benzyloxy)-3-(4-(4-(3-methoxy-4-nitrobenzylidene)piperidin-1-yl)phenyl)pyridine

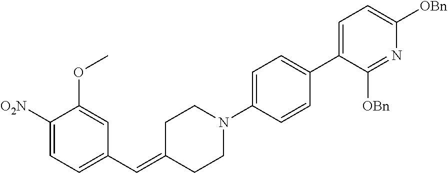 [SMILES: COc1cc(C=C2CCN(c3ccc(-c4ccc(OCc5ccccc5)nc4OCc4ccccc4)cc3)CC2)ccc1[N+](=O)[O-]]

A mixture of 2,6-bis(benzyloxy)-3-(4-bromophenyl)pyridine (171 mg, 0.384 mmol), 4-(3-methoxy-4-nitrobenzylidene)piperidine (100 mg, 0.403 mmol), Pd₂(dba)₃ (35 mg, 0.0384 mmol), 9,9-dimethyl-4,5-bis(diphenylphosphino)xanthene (44 mg, 0.0768 mmol), Cs₂CO₃ (250 mg, 0.768 mmol) in 1,4-dioxane (5.0 mL) was stirred in a microwave vial at 100° C. under atmosphere of N₂ for 16 h. After being cooled to room temperature, the mixture was filtrated through a pad of celite. The filtrate was concentrated under reduced pressure, the residue was purified by prep TLC with PE/EA (1:1) as eluent to obtain crude product. It was further purified by prep TLC with pure DCM as eluent to give the target product (98 mg, 42%). [M+H]⁺=614.3.

### Step 2: 3-(4-(4-(4-amino-3-methoxybenzyl)piperidin-1-yl)phenyl)piperidine-2,6-dione

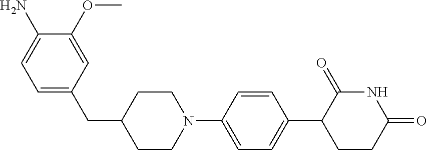 [SMILES: COc1cc(CC2CCN(c3ccc(C4CCC(=O)NC4=O)cc3)CC2)ccc1N]

A mixture 2,6-bis(benzyloxy)-3-(4-(4-(3-methoxy-4-nitrobenzylidene)piperidin-1-yl)phenyl)pyridine (98 mg, 0.160 mmol), 10% Pd/C (98 mg, 100 wt %) in EtOH/DCM/AcOH (2 mL/1 mL/30 μl) was stirred in a round flask at room temperature under hydrogen atmosphere for 16 h. The mixture was filtrated through a pad of celite, the filtrate was concentrated in vacuum to afford the crude product as colorless oil (65 mg, crude). [M+H]⁺=408.3.

### Step 3: 3-(4-(4-(4-((5-chloro-4-((2-(dimethylphosphoryl)phenyl)amino)pyrimidin-2-yl)amino)-3-methoxybenzyl)piperidin-1-yl)phenyl)piperidine-2,6-dione

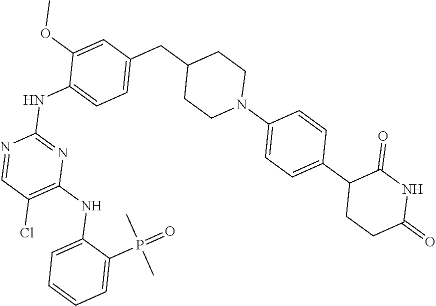 [SMILES: COc1cc(CC2CCN(c3ccc(C4CCC(=O)NC4=O)cc3)CC2)ccc1Nc1ncc(Cl)c(Nc2ccccc2P(C)(C)=O)n1]

A mixture 3-(4-(4-(4-amino-3-methoxybenzyl)piperidin-1-yl)phenyl)piperidine-2,6-dione (65 mg, 0.160 mmol), (2-((2,5-dichloropyrimidin-4-yl)amino)ph-enyl)dimethylphosphine oxide (51 mg, 0.160 mmol), TFA (120 uL, 1.60 mmol) in n-BuOH (2.0 mL) was stirred in a macrowave vial at 80° C. under nitrogen atmosphere for 16 h. After being cooled to room temperature, n-hexane (2.0 mL) was added, the precipitate was collected as crude product. It was further purified by prep HPLC to afford the product (10.5 mg, 9.6%). ¹H NMR (400 MHz, DMSO) δ 11.28 (s, 1H), 10.79 (s, 1H), 8.46 (s, 1H), 8.43 (s, 1H), 8.15 (s, 1H), 7.65-7.52 (m, 2H), 7.40 (t, J=7.8 Hz, 1H), 7.18-7.14 (m, 5H), 6.92 (s, 1H), 6.76 (d, J=8.1 Hz), 3.80 (s, 3H), 3.62 (d, J=11.9 Hz), 2.18-1.97 (m, 7H), 1.78-1.74 (m, 10H), 1.49 (s, 3H); [M+H]⁺=687.3.

### Example 200: 3-(4-(2-(4-(1-(4-((5-bromo-4-((8-(dimethylphosphoryl)-3-methylisoquinolin-7-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: ethyl 2-(3,5-difluoro-4-nitrophenyl)acetate

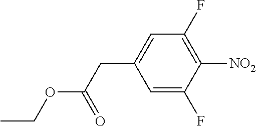 [SMILES: CCOC(=O)Cc1cc(F)c([N+](=O)[O-])c(F)c1]

A solution of 1,3-difluoro-2-nitrobenzene (50.0 g, 314.4 mmol) in NMP (300 mL) were cooled to −20° C. under N₂ atmosphere. Then a mixture of ethyl 2-chloroacetate (65.5 g, 534.7 mmol) and t-BuOK (121.0 g, 1.08 mol) in NMP (50 mL) was added slowly at −10° C. to −20° C. over 2 h. After being stirred for 2 h, the reaction was quenched by pouring into 1M HCl (200 mL) and ice-water. The mixture was extracted with EA (300 mL×3). The combined organic layer was washed by brine, dried with Na₂SO₄. The solution was concentrated in vacuum and the residue was purified by silica gel column chromatography (PE/EA=200/1 to 100/1) to provide product (13.7 g, 18%). ¹H NMR (400 MHz, CDCl₃) δ_{H} 7.06 (d, J=8.4 Hz, 2H), 4.20 (q, J=7.2 Hz, 2H), 3.65 (s, 2H), 1.28 (t, J=7.2 Hz, 3H).

### Step 2: ethyl 2-(4-amino-3,5-difluorophenyl)acetate

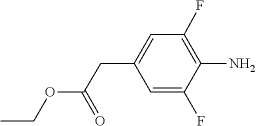

To a solution of ethyl 2-(3,5-difluoro-4-nitrophenyl)acetate (13.7 g, 56 mmol) in MeOH (150 mL) was added 10% Pd/C (1.5 g) at r.t. The mixture was stirred at r.t under H₂ atmosphere for 5 h. Filtrated on vacuum to remove Pd/C and concentrated in vacuum to provide the product (12.2 g), which was used in next step without further purification. ¹H NMR (400 MHz, DMSO_d₆) δ_{H} 6.82 (d, J=8.0 Hz, 2H), 5.69 (s, 2H), 4.06 (q, J=7.2 Hz, 2H), 3.52 (s, 2H), 1.17 (t, J=7.2 Hz, 3H). [M+H]⁺=216.4.

### Step 3: ethyl 2-(3,5-difluoro-4-iodophenyl)acetate

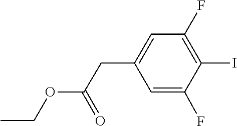

A solution of ethyl 2-(4-amino-3,5-difluorophenyl)acetate (12.2 g, 56 mmol) in MeCN (150 mL) was cooled to 0° C. under N₂ atmosphere and CuI (21.2 g, 112 mmol) was added. After stirring for 10 min, tert-butylnitrite (11.5 g, 112 mmol) was added dropwise over 30 min. Then the mixture was stirred at r.t for overnight. The reaction was quenched by pouring into water and extracted with EA (300 mL×3). All organic layers were combined and washed by brine, dried with Na₂SO₄. The solution was concentrated in vacuum and the residue was purified by silica gel column chromatography (PE/EA=500/1 to 100/1) to provide the product (8.8 g, 48%). [M+H]⁺=326.5.

### Step 4: ethyl 2-(4-(2,6-bis(benzyloxy)pyridin-3-yl)-3,5-difluorophenyl)acetate

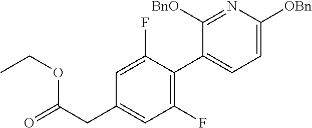 [SMILES: CCOC(=O)Cc1cc(F)c(-c2ccc(OCc3ccccc3)nc2OCc2ccccc2)c(F)c1]

To a solution of ethyl 2-(3,5-difluoro-4-iodophenyl)acetate (8.8 g, 27.0 mmol) in a mixed solvent of 1,4-dioxane/H₂O (100 mL/20 mL) were added K₂CO₃ (9.3 g, 67.4 mmol), 2,6-bis(benzyloxy)-3-(4,4,5,5-tetramethyl-1,3,2-dioxaborolan-2-yl)pyridine (14.6 g, 35.0 mol) and Pd(dppf)Cl₂ (2.9 g, 4.0 mmol) under N₂ atmosphere. The resulting solution was stirred for 6 h at 100° C. The mixture was diluted with water (300 mL) and extracted with EA (300 mL×3). All organic layers were combined and washed with brine (300 mL), dried over Na₂SO₄. The solution was concentrated in vacuum and the residue was purified by silica gel column chromatography (PE/EA=200/1) to give the product (8.2 g, 62%). ¹H NMR (400 MHz, CDCl₃) δ_{H} 7.49 (d, J=8.0 Hz, 1H), 7.40-7.24 (m, 10H), 6.90 (d, J=8.0 Hz, 2H), 6.47 (d, J=8.0 Hz, 1H), 5.38 (s, 2H), 5.33 (s, 2H), 4.19 (q, J=7.2 Hz, 2H), 3.61 (s, 2H), 1.28 (t, J=7.2 Hz, 3H).

### Step 5: 2-(4-(2,6-bis(benzyloxy)pyridin-3-yl)-3-yl)3,5-difluorophenyl)ethanol

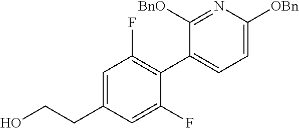 [SMILES: OCCc1cc(F)c(-c2ccc(OCc3ccccc3)nc2OCc2ccccc2)c(F)c1]

A solution of ethyl 2-(4-(2,6-bis(benzyloxy)pyridin-3-yl)-3,5-difluorophenyl)acetate (8.2 g, 16.7 mol) in THF (100 mL) was cooled to 0° C. under N₂ atmosphere and 1.5 M DIBAL-H (45 mL, 67.5 mol) in THF was added dropwise over 30 min. Then the mixture was stirred at r.t for 2 h. The reaction was quenched by pouring into water and extracted with EA (300 mL×3). All organic layers were combined and washed by brine, dried with Na₂SO₄. The solution was concentrated in vacuum and the residue was purified by column chromatography (PE/EA=10/1 to 3/1) to provide the product (6.6 g, 88%). ¹H NMR (400 MHz, CDCl₃) δ_{H}7.49 (d, J=8.0 Hz, 1H), 7.42-7.25 (m, 9H), 6.84 (d, J=8.0 Hz, 2H), 6.47 (d, J=8.0 Hz, 1H), 5.38 (s, 2H), 5.33 (s, 2H), 3.90 (m, 2H), 2.87 (t, J=6.4 Hz, 2H). [M+H]⁺=448.3.

### Step 6: 3-(2,6-difluoro-4-(2-hydroxyethyl)phenyl)piperidine-2,6-dione

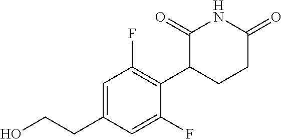 [SMILES: O=C1CCC(c2c(F)cc(CCO)cc2F)C(=O)N1]

To a solution of 2-(4-(2,6-bis(benzyloxy)pyridin-3-yl)-3,5-difluorophenyl)ethanol (6.6 g, 14.7 mmol) in DCM (150 mL) was added TFA (50 mL). After stirred overnight, the mixture was concentrated in vacuo. The residue was dissolved in MeOH (200 mL) and 10% Pd/C (1.0 g) was added. The resulted mixture was stirred for 2 days at r.t under H₂ atmosphere. The mixture was filtered and the filtrate was concentrated to give a residue which was purified by reversed flash C18 chromatography (ACN/water=0% to 30%) to give the title compound (2.1 g, 53%). [M+H]⁺=270.1.

### Step 7: 2-(4-(2,6-dioxopiperidin-3-yl)-3,5-difluorophenyl)acetaldehyde

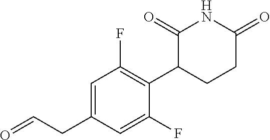 [SMILES: O=CCc1cc(F)c(C2CCC(=O)NC2=O)c(F)c1]

The titled compound (430 mg, 34%) was synthesized in a manner similar to that in Example 5 step 6 from 3-(2,6-difluoro-4-(2-hydroxyethyl)phenyl)piperidine-2,6-dione and IBX. [M+H]⁺=268.1.

### Step 8: N-(3-chloroisoquinolin-7-yl)-1,1-diphenylmethanimine

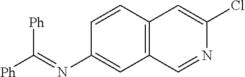 [SMILES: Clc1cc2ccc(N=C(c3ccccc3)c3ccccc3)cc2cn1]

To a solution of 7-bromo-3-chloroisoquinoline (2.5 g, 13.8 mmol) and diphenylmethanimine (3.35 g, 13.8 mmol) in dioxane (100 mL) was added Cs₂CO₃ (9 g, 27.6 mmol) at 20° C. Pd₂(dba)₃ (1.26 g, 1.4 mmol) and BINAP (1.72 g, 2.8 mmol) was added to the mixture at 20° C. The suspension was degassed under vacuum and purged with N₂ three times. Then the mixture was stirred at 100° C. for 14 hrs. The mixture was filtered and concentrated in vacuum. The residue was purified by column chromatography (DCM/MeOH=10/1 to 5/1). N-(3-chloroisoquinolin-7-yl)-1,1-diphenylmethanimine (3.3 g, 69.8%) was obtained. [M+H]⁺=343.1.

### Step 9: N-(3-methylisoquinolin-7-yl)-1,1-diphenylmethanimine

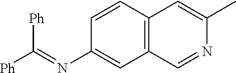 [SMILES: Cc1cc2ccc(N=C(c3ccccc3)c3ccccc3)cc2cn1]

To a solution of N-(3-chloroisoquinolin-7-yl)-1,1-diphenylmethanimine (3 g, 8.7 mmol) and 2,4,6-trimethyl-1,3,5,2,4,6-trioxatriborinane (2.2 g, 17.5 mmol) in dioxane (100 mL) was added K₂CO₃ (2.42 g, 17.5 mmol) at 20° C. Pd(dppf)Cl₂ (642 mg, 0.88 mmol) was added to the mixture at 20° C. The suspension was degassed under vacuum and purged with N, three times. Then the mixture was stirred at 100° C. for 12 hrs. The mixture was filtered and concentrated in vacuum. The residue was purified by column chromatography (PE/EA=10/1 to 1/1). N-(3-methylisoquinolin-7-yl)-1,1-diphenylmethanimine (1.9 g, 67.3%) was obtained. [M+H]⁺=323.2.

### Step 10: 3-methylisoquinolin-7-amine

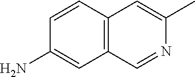

To a solution of N-(3-methylisoquinolin-7-yl)-1,1-diphenylmethanimine (1.9 g, 5.9 mmol) in THF (50 mL) was added HCl (aq. 12 N, 5 mL) at 20° C. Then the mixture was stirred at 20° C. for 1 hrs. Then the mixture was adjusted to pH=8 with NaHCO₃, and extracted with DCM (150 mL). The organic phase was washed with brine (150 mL), dried over Na₂SO₄, filtered and concentrated in vacuum. 3-methylisoquinolin-7-amine (630 mg, 67.8%) was obtained. [M+H]⁺=159.2.

### Step 11: 8-iodo-3-methylisoquinolin-7-amine

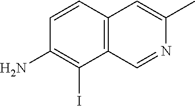

To a solution of 3-methylisoquinolin-7-amine (630 mg, 4 mmol) in HOAc (10 mL) was added ICl (775 mg, 4.8 mmol) at 20° C. Then the mixture was stirred at 20° C. for 1 hrs. Then the mixture was adjusted to pH=8 with sat. aq. NaHCO₃ solution and extracted with DCM (150 mL). The organic phase was washed with brine (150 mL), dried over Na₂SO₄, filtered and concentrated in vacuum. 8-iodo-3-methylisoquinolin-7-amine (900 mg, 79.6%) was obtained. [M+H]⁺=285.3.

### Step 12: (7-amino-3-methylisoquinolin-8-yl)dimethylphosphine oxide

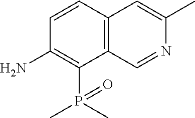 [SMILES: Cc1cc2ccc(N)c(P(C)(C)=O)c2cn1]

To a solution of 8-iodo-3-methylisoquinolin-7-amine (0.9 g, 3.2 mmol) and dimethylphosphine oxide (370 mg, 4.8 mmol) in dioxane (90 mL) was added K₃PO₄ (1.34 g, 6.3 mmol) at 20° C. And then Pd(OAc)₂ (71 mg, 0.3 mmol) and Xantphos (366 mg, 0.6 mmol) were added to the mixture at 20° C. The suspension was degassed under vacuum and purged with N₂ three times. Then the mixture was stirred at 100° C. for 14 hrs. The mixture was filtered and concentrated in vacuum. The residue was purified by column chromatography (DCM/MeOH=10/1 to 5/1). (7-amino-3-methylisoquinolin-8-yl)dimethylphosphine oxide (700 mg, 94%) was obtained. [M+H]⁺=235.2.

### Step 13: (7-((5-bromo-2-chloropyrimidin-4-yl)amino)-3-methylisoquinolin-8-yl)dimethylphosphine oxide

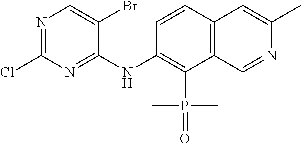 [SMILES: Cc1cc2ccc(Nc3nc(Cl)ncc3Br)c(P(C)(C)=O)c2cn1]

To a solution of (7-amino-3-methylisoquinolin-8-yl)dimethylphosphine oxide (400 mg, 1.7 mmol) in DMF (10 mL) was added 5-bromo-2,4-dichloropyrimidine (158 mg, 2.6 mmol) at 0° C. And then LiHMDS (1 N, 1.88 mL, 314 mmol) was added to the reaction mixture at 0° C. The mixture was stirred at 20° C. for 3 hrs. Water (100 mL) was poured into the mixture, which was further extracted with EtOAc (100 mL). The combined organic phase was washed with brine (100 mL), dried over Na₂SO₄, filtered and concentrated in vacuum. The residue was purified by column chromatography (DCM/MeOH=10/1 to 5/1). (7-((5-bromo-2-chloropyrimidin-4-yl)amino)-3-methylisoquinolin-8-yl)dimethylphosphine oxide (180 mg, 24.8%) was obtained. [M+H]⁺=425.1.

### Step 14: (7-((5-bromo-2-((5-ethyl-2-methoxy-4-(4-(piperazin-1-yl)piperidin-1-yl)phenyl)amino)pyrimidin-4-yl)amino)-3-methylisoquinolin-8-yl)dimethylphosphine oxide

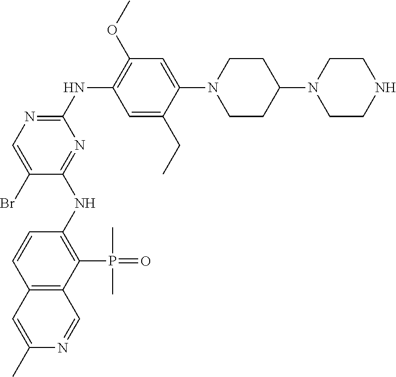 [SMILES: CCc1cc(Nc2ncc(Br)c(Nc3ccc4cc(C)ncc4c3P(C)(C)=O)n2)c(OC)cc1N1CCC(N2CCNCC2)CC1]

To a solution of (7-((5-bromo-2-chloropyrimidin-4-yl)amino)-3-methylisoquinolin-8-yl)dimethylphosphine oxide (180 mg, 0.4 mmol) in n-BuOH (10 mL) was added tert-butyl 4-(1-(4-amino-2-ethyl-5-methoxyphenyl)piperidin-4-yl)piperazine-1-carboxylate (This intermediate was prepared according to the same manner in Example 23 step 3) (177 mg, 0.4 mmol) at 20° C. 4-methylbenzenesulfonic acid (218 mg, 1.3 mmol) was added to the reaction mixture at 20° C. Then the mixture was stirred at 100° C. for 13 hrs. Water (100 mL) was poured into the mixture. Then the mixture was adjusted to pH=8 with sat. aq. NaHCO₃ solution and extracted with DCM (150 mL). The organic phase was washed with brine (150 mL), dried over Na₂SO₄, filtered and concentrated in vacuum. The residue was purified by column chromatography (DCM/MeOH=10/1 to 5/1). (7-((5-bromo-2-((5-ethyl-2-methoxy-4-(4-(piperazin-1-yl)piperidin-1-yl)phenyl)amino)pyrimidin-4-yl)amino)-3-methylisoquinolin-8-yl)dimethylphosphine oxide (120 mg, 40.1%) was obtained. [M+H]⁺=707.2.

### Step 15: 3-(4-(2-(4-(1-(4-((5-bromo-4-((8-(dimethylphosphoryl)-3-methylisoquinolin-7-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

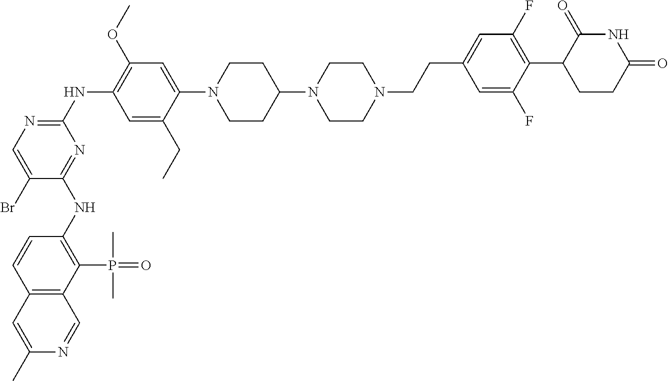 [SMILES: CCc1cc(Nc2ncc(Br)c(Nc3ccc4cc(C)ncc4c3P(C)(C)=O)n2)c(OC)cc1N1CCC(N2CCN(CCc3cc(F)c(C4CCC(=O)NC4=O)c(F)c3)CC2)CC1]

To a solution of (7-((5-bromo-2-((5-ethyl-2-methoxy-4-(4-(piperazin-1-yl)piperidin-1-yl)phenyl)amino)pyrimidin-4-yl)amino)-3-methylisoquinolin-8-yl)dimethylphosphine oxide (30 mg, 0.04 mmol) in DCM (5 mL) was added 2-(4-(2,6-dioxopiperidin-3-yl)-3,5-difluorophenyl)acetaldehyde (11 mg, 0.04 mmol) at 20° C. The mixture was stirred at 20° C. for 1 hrs and STAB (18 mg, 0.08 mmol) was added. Then the mixture was stirred at 20° C. for 12 hrs. Water (10 mL) was poured into the mixture. Then the mixture was extracted with DCM (50 mL). The organic phase was washed with brine (50 mL), dried over Na₂SO₄, filtered and concentrated in vacuum. The residue was purified by prep-HPLC (C-18 column chromatography (0.1% FA in water: acetonitrile=90: 10˜ 60: 40 gradient elution) to afford the product (7.2 mg, 17.6%). ¹H NMR (500 MHz, DMSO) δ 11.77 (s, 1H), 10.96 (s, 1H), 9.51 (s, 1H), 8.26 (d, J=19.8 Hz, 2H), 7.99-7.81 (m, 2H), 7.67 (s, 1H), 7.47 (d, J=11.3 Hz, 1H), 7.09 (dd, J=20.7, 9.1 Hz, 2H), 6.73 (s, 1H), 4.20 (d, J=7.4 Hz, 1H), 3.77 (s, 3H), 3.52 (s, 2H), 3.17-2.89 (m, 7H), 2.79 (s, 3H), 2.72-2.54 (m, 9H), 2.36 (s, 2H), 2.21 (d, J=73.4 Hz, 4H), 2.05 (d, J=13.3 Hz, 7H), 1.79 (s, 2H), 0.74 (s, 3H). [M+H]⁺=958.2.

### Example 201: 3-(4-(2-(4-(1-(4-((5-bromo-4-((5-(dimethylphosphoryl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: 5-iodoquinolin-6-amine

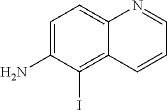 [SMILES: Nc1ccc2ncccc2c1I]

The titled compound (3.1 g, 85%) was prepared in a manner similar to that in Example 200 step 11 from quinolin-6-amine. [M+H]⁺=271.0

### Step 2: (6-aminoquinolin-5-yl)dimethylphosphine oxide

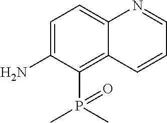

The titled compound (2.4 g, 95%) was prepared in a manner similar to that in Example 200 step 12 from 5-iodoquinolin-6-amine. [M+H]⁺=221.2

### Step 3: (6-((5-bromo-2-chloropyrimidin-4-yl)amino)quinolin-5-yl)dimethylphosphine oxide

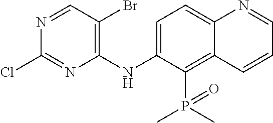 [SMILES: CP(C)(=O)c1c(Nc2nc(Cl)ncc2Br)ccc2ncccc12]

The titled compound (1.2 g, 75%) was prepared in a manner similar to that in Example 208 step 6 from (6-aminoquinolin-5-yl)dimethylphosphine oxide and 5-bromo-2,4-dichloropyrimidine. [M+H]⁺=411.0

### Step 4: (6-((5-bromo-2-((5-ethyl-2-methoxy-4-(4-(piperazin-1-yl)piperidin-1-yl)phenyl)amino)pyrimidin-4-yl)amino)quinolin-5-yl)dimethylphosphine oxide

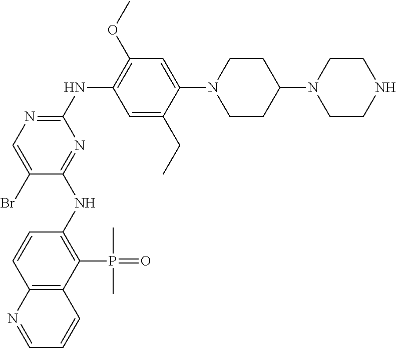 [SMILES: CCc1cc(Nc2ncc(Br)c(Nc3ccc4ncccc4c3P(C)(C)=O)n2)c(OC)cc1N1CCC(N2CCNCC2)CC1]

To a solution of (6-((5-bromo-2-chloropyrimidin-4-yl)amino)quinolin-5-yl)dimethylphosphine oxide (500 mg, 1.22 mmol) and tert-butyl 4-(1-(4-amino-2-ethyl-5-methoxyphenyl)piperidin-4-yl)piperazine-1-carboxylate (510 mg, 1.22 mmol) in t-BuOH (30 mL) was added MsOH (468 mg, 4.88 mmol) at room temperature. The resulting mixture was stirred at 100° C. for 16 hours. The resulting solution was concentrated and basified with sat. aq. NaHCO₃ solution, then extracted with DCM (2×80 mL). The combined organic layers were dried over anhydrous Na₂SO₄, filtered and evaporated in vacuum to afford the crude residue, which was purified by silica gel column chromatography (DCM: MeOH=100: 0˜5: 1 gradient elution) to give the desired product (270 mg, 32%). [M+H]⁺=693.5.

### Step 5: 3-(4-(2-(4-(1-(4-((5-bromo-4-((5-(dimethylphosphoryl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

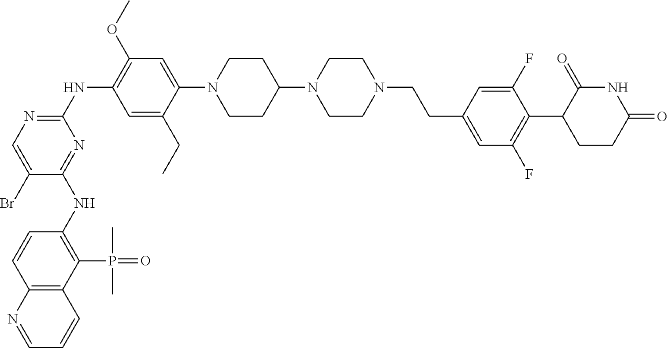 [SMILES: CCc1cc(Nc2ncc(Br)c(Nc3ccc4ncccc4c3P(C)(C)=O)n2)c(OC)cc1N1CCC(N2CCN(CCc3cc(F)c(C4CCC(=O)NC4=O)c(F)c3)CC2)CC1]

To a solution of (6-((5-bromo-2-((5-ethyl-2-methoxy-4-(4-(piperazin-1-yl)piperidin-1-yl)phenyl)amino)pyrimidin-4-yl)amino)quinolin-5-yl)dimethylphosphine oxide (30 mg, 0.04 mmol) in DCM (6 mL) was added 2-(4-(2,6-dioxopiperidin-3-yl)-3,5-difluorophenyl)acetaldehyde (22 mg, 0.08 mmol) at room temperature. After stirring at room temperature for 1h, NaBH(OAc)₃ (26 mg, 0.12 mmol) was added and the resulting mixture was stirred at room temperature overnight. The reaction was quenched with water (10 mL) and extracted with DCM (2×20 mL). The combined organic layers were dried over anhydrous Na₂SO₄, filtered and evaporated in vacuum to afford the crude residue, which was purified by silica gel column chromatography (DCM: MeOH=100: 0˜10: 1 gradient elution) to give an impure product, which was further purified with pre-HPLC with C-18 column chromatography (0.1% FA in water: acetonitrile=90: 10˜ 60: 40 gradient elution) to give the desired product (15 mg, 37%). ¹H NMR (400 MHz, DMSO) δ_{H} 11.97 (s, 1H), 10.95 (s, 1H), 8.86 (d, J=3.1 Hz, 1H), 8.64 (d, J=8.8 Hz, 1H), 8.40 (s, 1H), 8.24 (s, 1H), 8.02-7.92 (m, 2H), 7.55 (dd, J=8.7, 4.1 Hz, 1H), 7.41 (s, 1H), 7.03 (d, J=10.1 Hz, 2H), 6.75 (s, 1H), 4.20 (dd, J=12.8, 4.9 Hz, 1H), 3.77 (s, 3H), 2.95 (d, J=11.0 Hz, 2H), 2.85-2.73 (m, 3H), 2.65 (t, J=10.9 Hz, 2H), 2.54 (d, J=7.5 Hz, 7H), 2.48-2.26 (m, 7H), 2.18-2.08 (m, 1H), 2.02 (d, J=13.3 Hz, 7H), 1.84 (d, J=11.2 Hz, 2H), 1.55 (d, J=8.9 Hz, 2H), 0.76 (s, 3H); [M+H]⁺=944.5.

### Example 202: 3-(4-(4-(2-(4-(1-(4-((5-bromo-4-((5-(dimethylphosphoryl)quinolin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

### Step 1: 2,6-bis(benzyloxy-3-(4,4,5,5-tetramethyl-1,3,2-dioxaborolan-2-yl)pyridine

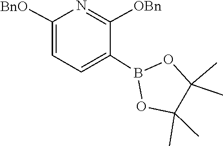 [SMILES: CC1(C)OB(c2ccc(OCc3ccccc3)nc2OCc2ccccc2)OC1(C)C]

A mixture of 2,6-bis(benzyloxy)-3-bromopyridine (15 g, 40.65 mmol) and 4,4,4′,4′,5,5,5′,5′-octamethyl-2,2′-bi(1,3,2-dioxaborolane) (12.6 g, 49.61 mmol), Pd(dppf)Cl₂ (3.32 g, 4.07 mmol), KOAc (12 g, 122.45 mmol) in dioxane (200 mL) was stirred overnight at 100° C. under nitrogen atmosphere. The resulting mixture was filtered, the filter cake was washed with MeOH and DCM. The filtrate was concentrated under reduced pressure. The residue was purified by silica gel column chromatography, eluted with PE/EtOAc (8:1) to afford the product (9.00 g, 53%). m/z[M+H]⁺=418.3.

### Step 2: 2,6-bis(benzyloxy)-3-(4-bromo-2,6-difluorophenyl)pyridine

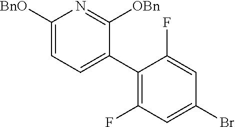 [SMILES: Fc1cc(Br)cc(F)c1-c1ccc(OCc2ccccc2)nc1OCc1ccccc1]

A mixture of 2,6-bis(benzyloxy)-3-(4,4,5,5-tetramethyl-1,3,2-dioxaborolan-2-yl)pyridine (9.00 g, 21.56 mmol) and 5-bromo-1,3-difluoro-2-iodobenzene (6.88 g, 21.57 mmol), K₂CO₃ (10.43 g, 75.48 mmol), Pd(dppf)Cl₂ (789 mg, 1.078 mmol) in dioxane (90 mL) and H₂O (30 mL) was stirred for 16h at 100° C. under nitrogen atmosphere. The resulting mixture was extracted with EtOAc (50 mL×3). The combined organic layers were washed with brine, dried over anhydrous Na₂SO₄. After filtration, the filtrate was concentrated under reduced pressure. The residue was purified by silica gel column chromatography, eluted with PE/EtOAc (5:1) to afford the product (4 g, 38%). [M+H]⁺=482.4.

### Step 3: ethyl 2-(1-(4-(2,6-bis(benzyloxy)pyridin-3-yl)-3,5-difluorophenyl)piperidin-4-yl)acetate

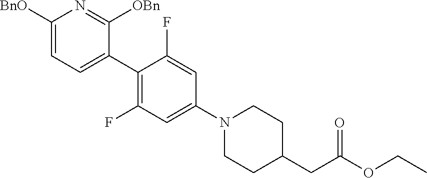 [SMILES: CCOC(=O)CC1CCN(c2cc(F)c(-c3ccc(OCc4ccccc4)nc3OCc3ccccc3)c(F)c2)CC1]

A mixture of 2,6-bis(benzyloxy)-3-(4-bromo-2,6-difluorophenyl)pyridine (4.00 g, 8.29 mmol), ethyl 2-(piperidin-4-yl)acetate (2.13 g, 12.43 mmol), Cs₂CO₃ (8.11 g, 24.89 mmol), DavePhos (652.7 mg, 1.659 mmol), Pd₂(dba)₃ (759.4 mg, 0.829 mmol) in 2-methyl-THF (50 mL) and H₂O (5 mL) was stirred overnight at 100° C. under nitrogen atmosphere. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (1:1) to afford the product (2 g, 42%). [M+1]⁺=573.1.

### Step 4: 2-(1-(4-(2,6-bis(benzyloxy)pyridin-3-yl)-3,5-difluorophenyl)piperidin-4-yl)ethan-1-ol

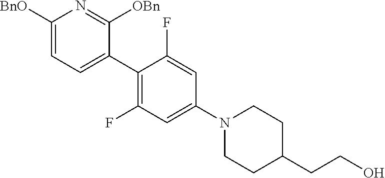 [SMILES: OCCC1CCN(c2cc(F)c(-c3ccc(OCc4ccccc4)nc3OCc3ccccc3)c(F)c2)CC1]

To a stirred mixture of ethyl 2-(1-(4-(2,6-bis(benzyloxy)pyridin-3-yl)-3,5-difluorophenyl)piperidin-4-yl)acetate (2.00 g, 3.49 mmol) in THF (50 mL) was added LiBH₄ (1.52 g, 69.77 mmol) in portions at room temperature overnight. The reaction was quenched with water (20 mL) at room temperature. The resulting mixture was extracted with EtOAc (3×50 mL). The combined organic layers were washed with brine, dried over anhydrous Na₂SO₄. After filtration, the filtrate was concentrated under reduced pressure to afford the product (1.8 g, 97%) [M+1]⁺=531.2.

### Step 5: 3-(2,6-difluoro-4-(4-(2-hydroxyethyl)piperidin-1-yl)phenyl)piperidine-2,6-dione

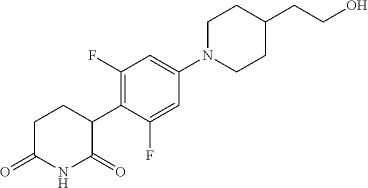 [SMILES: O=C1CCC(c2c(F)cc(N3CCC(CCO)CC3)cc2F)C(=O)N1]

To a stirred mixture of 2-(1-(4-(2,6-bis(benzyloxy)pyridin-3-yl)-3,5-difluorophenyl)piperidin-4-yl)ethan-1-ol (1.80 g, 3.39 mmol) and Pd/C (1 g, 10% wt) in EtOH (20 mL) and DCM (20 mL) were added AcOH (20 mL) at rt and stirred at 40° C. under hydrogen atmosphere overnight. The resulting mixture was filtered, the filter cake was washed with MeOH (20 mL). The filtrate was concentrated under reduced pressure to afford the product (1.2 g, 100%). [M+1]⁺=353.3.

### Step 6: 2-(1-(4-(2,6-dioxopiperidin-3-yl)-3,5-difluorophenyl)piperidin-4-yl)acetaldehyde

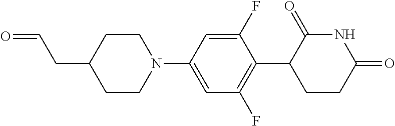 [SMILES: O=CCC1CCN(c2cc(F)c(C3CCC(=O)NC3=O)c(F)c2)CC1]

The titled compound (1.4 g, 92%) was prepared in a manner similar to that in Example 291 step 6 from 3-(2,6-difluoro-4-(4-(2-hydroxyethyl)piperidin-1-yl)phenyl)piperidine-2,6-dione and IBX. [M+H]⁺=351.2.

### Step 7: 3-(4-(4-(2-(4-(1-(4-((5-bromo-4-((5-(dimethylphosphoryl)quinolin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

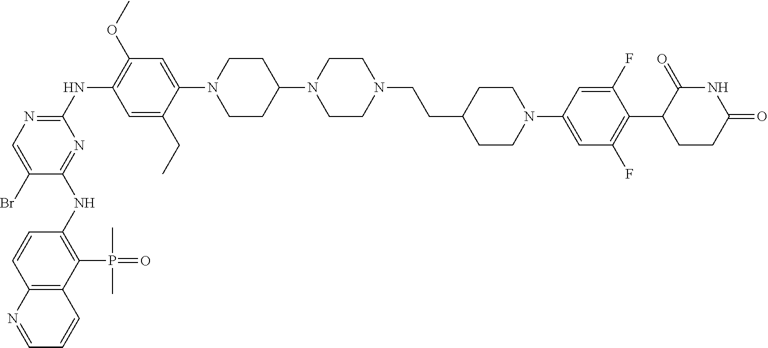 [SMILES: CCc1cc(Nc2ncc(Br)c(Nc3ccc4ncccc4c3P(C)(C)=O)n2)c(OC)cc1N1CCC(N2CCN(CCC3CCN(c4cc(F)c(C5CCC(=O)NC5=O)c(F)c4)CC3)CC2)CC1]

The titled compound was synthesized in the procedures similar to Example 200. ¹H NMR (400 MHz, DMSO)^{6H} 11.96 (s, 1H), 10.86 (s, 1H), 8.85 (d, J=4.0 Hz, 1H), 8.63 (d, J=8.4 Hz, 1H), 8.39 (s, 1H), 8.23 (s, 1H), 8.03-7.90 (m, 2H), 7.54 (dd, J=8.7, 4.0 Hz, 1H), 7.40 (s, 1H), 6.74 (s, 1H), 6.60 (d, J=12.8 Hz, 2H), 4.06-4.02 (m, 1H), 3.76 (s, 5H), 2.94 (d, J=10.4 Hz, 2H), 2.79-2.64 (m, 10H), 2.31 (s, 7H), 2.14-1.98 (m, 10H), 1.83 (d, J=10.3 Hz, 2H), 1.70 (t, J=14.9 Hz, 2H), 1.59-1.43 (m, 3H), 1.38 (d, J=6.8 Hz, 2H), 1.17 (d, J=13.6 Hz, 2H), 0.75 (s, 3H); [M+H]⁺=1027.7.

### Example 203: 3-(4-(4-(2-(3-(4-(4-((5-bromo-4-((5-(dimethylphosphoryl)quinoxalin-6-yl)amino)pyrimidin-2-yl)amino)-2-fluorophenyl)piperazin-1-yl)azetidin-1-yl)ethyl)piperidin-1-yl)-2-fluorophenyl)piperidine-2,6-dione

### Step 1: tert-butyl 3-(4-(2-fluoro-4-nitrophenyl)piperazin-1-yl)azetidine-1-carboxylate

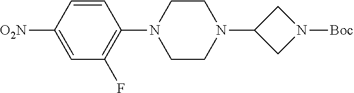 [SMILES: CC(C)(C)OC(=O)N1CC(N2CCN(c3ccc([N+](=O)[O-])cc3F)CC2)C1]

A mixture of 1,2-difluoro-4-nitrobenzene (1.20 g, 5.0 mmol), tert-butyl 3-(piperazin-1-yl)azetidine-1-carboxylate (0.80 g, 5.0 mmol), K₂CO₃ (1.38 g, 10.0 mmol) in ACN (80 mL) was stirred in a round bottom flask at 80° C. for 18 hours. The mixture was evaporated in vacuum to afford the crude product, which was further purified with silica gel column chromatography (PE: EA=100: 1˜ 1: 100 gradient elution) to give the title product (0.86 g, 45%). [M+H]⁺=380.8.

### Step 2: tert-butyl 3-(4-(4-amino-2-fluorophenyl)piperazin-1-yl)azetidine-1-carboxylate

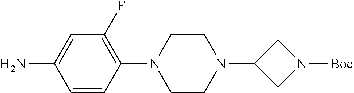 [SMILES: CC(C)(C)OC(=O)N1CC(N2CCN(c3ccc(N)cc3F)CC2)C1]

To a solution of tert-butyl 3-(4-(4-amino-2-fluorophenyl)piperazin-1-yl)azetidine-1-carboxylate (0.86 g, 2.26 mmol) in MeOH (40 mL) was added Pd/C (86 mg) and stirred at 25° C. for 18 hours under one balloon H₂. The mixture was filtered and evaporated in vacuum to afford the crude product (0.60 g, 80%). [M+H]⁺=350.8.

### Step 3: (6-((2-((4-(4-(azetidin-3-yl)piperazin-1-yl)-3-fluorophenyl)amino)-5-bromopyrimidin-4-yl)amino)quinoxalin-5-yl)dimethylphosphine oxide

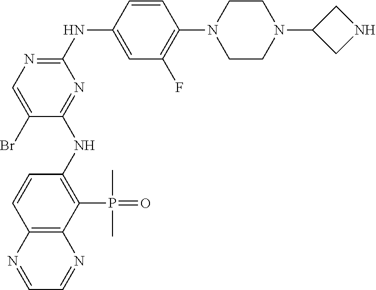 [SMILES: CP(C)(=O)c1c(Nc2nc(Nc3ccc(N4CCN(C5CNC5)CC4)c(F)c3)ncc2Br)ccc2nccnc12]

A mixture of tert-butyl 3-(4-(4-amino-2-fluorophenyl)piperazin-1-yl)azetidine-1-carboxylate (0.62 g, 1.77 mmol) and (6-((5-bromo-2-chloropyrimidin-4-yl)amino)quinoxalin-5-yl)dimethylphosphine oxide (0.44 g, 1.07 mmol) in isopropanol (30 mL) and MsOH (1 mL) was stirred in a round bottom flask at 100° C. for 18 hours. Then the mixture was evaporated in vacuum to afford the crude product, which was purified with C18 column chromatography (0.5% FA in water: ACN=90: 10˜ 55: 45 gradient elution) to give the title product (0.27 g, 40%). [M+H]⁺=625.8.

### Step 4: 3-(4-(4-(2-(3-(4-(4-((5-bromo-4-((5-(dimethylphosphoryl)quinoxalin-6-yl)amino)pyrimidin-2-yl)amino)-2-fluorophenyl)piperazin-1-yl)azetidin-1-yl)ethyl)piperidin-1-yl)-2-fluorophenyl)piperidine-2,6-dione

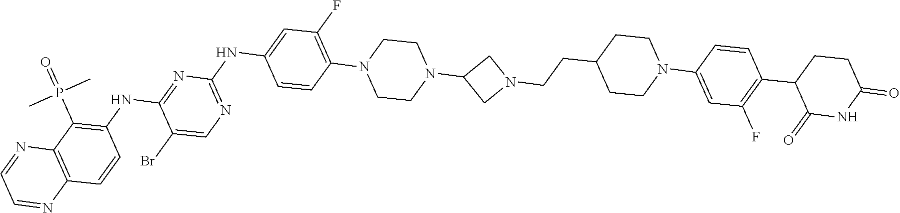 [SMILES: CP(C)(=O)c1c(Nc2nc(Nc3ccc(N4CCN(C5CN(CCC6CCN(c7ccc(C8CCC(=O)NC8=O)c(F)c7)CC6)C5)CC4)c(F)c3)ncc2Br)ccc2nccnc12]

A mixture of (6-((2-((4-(4-(azetidin-3-yl)piperazin-1-yl)-3-fluorophenyl)amino)-5-bromopyrimidin-4-yl)amino)quinoxalin-5-yl)dimethylphosphine oxide (30 mg, 0.05 mmol) in DCM (5 mL) and MeOH (1 mL) was added 2-(1-(4-(2,6-dioxopiperidin-3-yl)-3-fluorophenyl)piperidin-4-yl)acetaldehyde (this material was obtained through the similar method of example 22) (30 mg, 0.10 mmol) then stirred in a round bottom flask at room temperature for 1 hour. The mixture was added NaBH(OAc)₃ (106 mg, 0.50 mmol) and stirred in a round bottom flask at room temperature 18 hours. Then the mixture was evaporated in vacuum to afford the crude product, which was purified with Pre-TLC (DCM: MeOH=10:1) to give the product (12 mg, 25%). ¹H NMR (400 MHz, DMSO) δ 12.72 (s, 1H), 10.81 (s, 1H), 9.58 (s, 1H), 9.02-8.81 (m, 3H), 8.37 (s, 1H), 8.13 (d, J=9.5 Hz, 1H), 7.63 (d, J=15.0 Hz, 1H), 7.29-7.21 (m, 1H), 7.10-7.02 (m, 1H), 7.00-6.93 (m, 1H), 6.71 (d, J=11.0 Hz, 2H), 3.94-3.80 (m, 2H), 3.75-3.67 (m, 2H), 3.04-2.87 (m, 6H), 2.76-2.59 (m, 4H), 2.48-2.42 (m, 4H), 2.24-2.10 (m, 2H), 1.99-1.91 (m, 2H), 1.75-1.67 (m, 2H), 1.57-1.42 (m, 2H), 1.40-1.33 (m, 2H), 1.29-1.11 (m, 7H), 0.92-0.73 (m, 3H). [M+H]⁺=942.2.

### Example 204: 3-(4-(4-(2-(3-(4-(4-((5-bromo-4-((5-(dimethylphosphoryl)quinoxalin-6-yl)amino)pyrimidin-2-yl)amino)-5-methoxy-2-methylphenyl)piperazin-1-yl)azetidin-1-yl)ethyl)piperidin-1-yl)-2,6-difluorophenyl)piperidine-2,6-dione

### Step 1: tert-butyl 4-(5-methoxy-2-methyl-4-nitrophenyl)piperazine-1-carboxylate

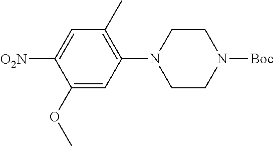 [SMILES: COc1cc(N2CCN(C(=O)OC(C)(C)C)CC2)c(C)cc1[N+](=O)[O-]]

A mixture of 1-fluoro-5-methoxy-2-methyl-4-nitrobenzene (1.00 g, 5.4 mmol), tert-butyl piperazine-1-carboxylate (1.08 g, 5.8 mmol) and DIPEA (1.40 g, 10.8 mmol) in DMSO (20 mL) was stirred in a round bottom flask at 100° C. for 60 hours. The mixture was added brine (100 mL), extracted with EA (100 mL×3). The organics were evaporated in vacuum to afford the crude product, which was further purified with silica gel column chromatography (PE: EA=100: 1˜ 1:1 gradient elution) to give product (1.25 g, 66%). [M+H]⁺=351.9.

### Step 2: 1-(5-methoxy-2-methyl-4-nitrophenyl)piperazine

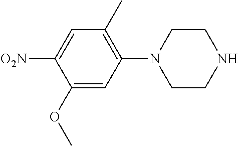 [SMILES: COc1cc(N2CCNCC2)c(C)cc1[N+](=O)[O-]]

To a solution of tert-butyl 4-(5-methoxy-2-methyl-4-nitrophenyl)piperazine-1-carboxylate (1.25 g, 3.6 mmol) in DCM (100 mL) was added 4N HCl in dioxane (10 mL) and stirred at 25° C. for 2 hours. The mixture was filtered, the filter cake was washed with DCM and dried in vacuum to afford the product (1.14 g, 90%). [M+H]⁺=251.8.

### Step 3: tert-butyl 3-(4-(5-methoxy-2-methyl-4-nitrophenyl)piperazin-1-yl)azetidine-1-carboxylate

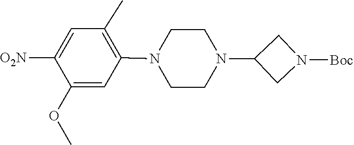 [SMILES: COc1cc(N2CCN(C3CN(C(=O)OC(C)(C)C)C3)CC2)c(C)cc1[N+](=O)[O-]]

A mixture of 1-(5-methoxy-2-methyl-4-nitrophenyl)piperazine (575 mg, 2.0 mmol) and tert-butyl 3-oxoazetidine-1-carboxylate (342 mg, 2 mmol) in dichloromethane (40 mL) and EtOH (5 mL) was added Ti(iPrO)₄ (2.84 g, 10.0 mmol) stirred in a round bottom flask at room temperature for 2 hours. The mixture was added NaBH(OAc)₃ (2.12 g, 10.0 mmol) and stirred at 50° C. for 18 hours. Then the mixture was washed with water (50 mL×3), then evaporated in vacuum to afford the crude product, which was further purified with silica gel column chromatography (PE: EA=100: 1˜ 85: 15 gradient elution) to give product (1.20 g, crude). [M+H]⁺=406.8.

### Step 4: tert-butyl 3-(4-(4-amino-5-methoxy-2-methylphenyl)piperazin-1-yl)azetidine-1-carboxylate

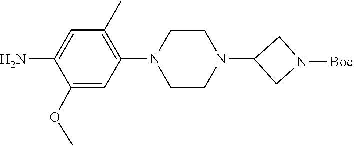 [SMILES: COc1cc(N2CCN(C3CN(C(=O)OC(C)(C)C)C3)CC2)c(C)cc1N]

A mixture of tert-butyl 3-(4-(5-methoxy-2-methyl-4-nitrophenyl)piperazin-1-yl)azetidine-1-carboxylate (1.02 g, 2 mmol) in MeOH (20 mL) was added Pd/C (200 mg) stirred in a round bottom flask at room temperature under one balloon H₂ for 18 hours. The mixture was filtered and washed with MeOH, then evaporated in vacuum to afford the crude product, which was further purified with silica gel column chromatography (PE: EA=100: 1˜ 85: 15 gradient elution) to give product (0.44 g, 58%). [M+H]1=376.8.

### Step 5: (6-((2-((4-(4-(azetidin-3-yl)piperazin-1-yl)-2-methoxy-5-methylphenyl)amino)-5-bromopyrimidin-4-yl)amino)quinoxalin-5-yl)dimethylphosphine oxide

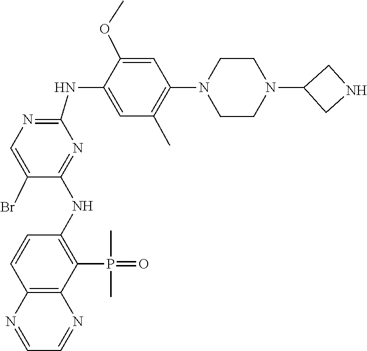 [SMILES: COc1cc(N2CCN(C3CNC3)CC2)c(C)cc1Nc1ncc(Br)c(Nc2ccc3nccnc3c2P(C)(C)=O)n1]

A mixture of tert-butyl 3-(4-(4-amino-5-methoxy-2-methylphenyl)piperazin-1-yl)azetidine-1-carboxylate (0.44 g, 1.17 mmol) and (6-((5-bromo-2-chloropyrimidin-4-yl)amino)quinoxalin-5-yl)dimethylphosphine oxide (0.21 g, 0.50 mmol) in isopropanol (20 mL) and MsOH (0.196 g, 2.0 mmol) was stirred in a round bottom flask at 100° C. for 36 hours. Then the mixture was evaporated in vacuum to afford the crude product, which was purified with C18 column chromatography (0.5% FA in water: ACN=90: 10˜ 55: 45 gradient elution) to give the title product (90 mg, 6%). [M+H]⁺=651.8.

### Step 6: 3-(4-(4-(2-(3-(4-(4-((5-bromo-4-((5-(dimethylphosphoryl)quinoxalin-6-yl)amino)pyrimidin-2-yl)amino)-5-methoxy-2-methylphenyl)piperazin-1-yl)azetidin-1-yl)ethyl)piperidin-1-yl)-2,6-difluorophenyl)piperidine-2,6-dione

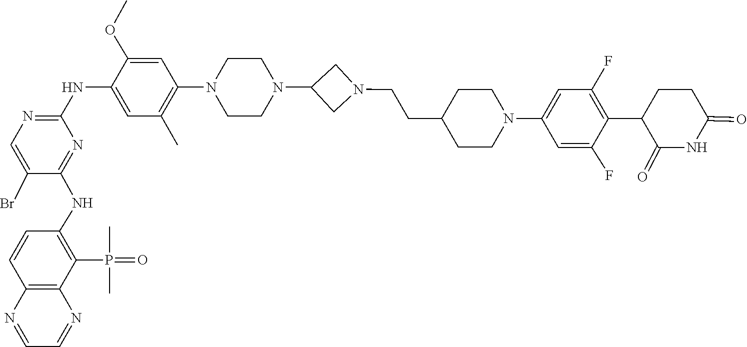 [SMILES: COc1cc(N2CCN(C3CN(CCC4CCN(c5cc(F)c(C6CCC(=O)NC6=O)c(F)c5)CC4)C3)CC2)c(C)cc1Nc1ncc(Br)c(Nc2ccc3nccnc3c2P(C)(C)=O)n1]

A mixture of (6-((2-((4-(4-(azetidin-3-yl)piperazin-1-yl)-2-methoxy-5-methylphenyl)amino)-5-bromopyrimidin-4-yl)amino)quinoxalin-5-yl)dimethylphosphine oxide (50 mg, 0.07 mmol) in dichloromethane (10 mL) and MeOH (1 mL) was added 2-(1-(4-(2,6-dioxopiperidin-3-yl)-3,5-difluorophenyl)piperidin-4-yl)acetaldehyde (35 mg, 0.10 mmol) then stirred in a round bottom flask at room temperature for 1 hour. The mixture was added NaBH(OAc)₃ (106 mg, 0.50 mmol) and stirred in a round bottom flask at room temperature 18 hours. Then the mixture was evaporated in vacuum to afford the crude product, which was purified with Pre-TLC (DCM: MeOH=7:1) to give the product (3.5 mg, 5%). ¹H NMR (400 MHz, DMSO) δ 12.69 (s, 1H), 10.86 (s, 1H), 8.97-8.76 (m, 3H), 8.30-8.25 (m, 2H), 8.24-8.20 (m, 1H), 7.92 (d, J=9.0 Hz, 1H), 7.35 (s, 1H), 6.79 (s, 1H), 6.60 (d, J=13.0 Hz, 2H), 4.04 (dd, J=12.5, 5.0 Hz, 1H), 3.78 (s, 3H), 3.76-3.69 (m, 2H), 3.42 (t, J=6.0 Hz, 3H), 2.97-2.91 (m, 1H), 2.90-2.83 (m, 4H), 2.82-2.74 (m, 3H), 2.74-2.65 (m, 2H), 2.46-2.33 (m, 3H), 2.12-2.06 (m, 4H), 2.04 (s, 3H), 2.01 (s, 3H), 1.99-1.92 (m, 1H), 1.77-1.65 (m, 2H), 1.54-1.41 (m, 1H), 1.29-1.07 (m, 4H). [M+H]⁺=985.8.

### Example 291: 3-(4-(2-((S)-4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)-2-(hydroxymethyl)piperazin-1-yl)ethyl)-2-fluorophenyl)piperidine-2,6-dione

### Step 1: 2-(4-bromo-3-fluorophenyl)ethan-1-ol

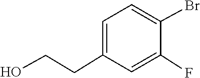 [SMILES: OCCc1ccc(Br)c(F)c1]

To a solution of 2-(4-bromo-3-fluorophenyl)acetic acid (45.0 g, 193 mmol) in THF (270 mL) was added BH₃-THF (1 M, 386 mL) at 0° C. Then the mixture was stirred at 20° C. for 2 hrs. Under cooling with ice, MeOH (250 mL) was dropwise added until there was no foaming in the system and the solvent was distilled off under reduced pressure. To the resulting reside, water (50.0 mL) was added for extraction with EtOAc (1000.0 mL). The combined organic phase was washed with brine (40.0 mL), dried over Na₂SO₄, filtered and concentrated in vacuum. 2-(4-bromo-3-fluorophenyl)ethan-1-ol (38.0 g, 89.8%) was obtained. ¹HNMR (400 MHz, CDCl₃-d) δ ppm 7.45 (t, J=7.72 Hz, 1H), 7.00 (dd, J=9.48, 1.76 Hz, 1H), 6.86-6.92 (m, 1H), 3.82 (t, J=6.50 Hz, 2H), 2.80 (t, J=6.50 Hz, 2H), 2.03 (s, 1H); [M+H]⁺=219.3.

### Step 2: (4-bromo-3-fluorophenethoxy)(tert-butyl)dimethylsilane

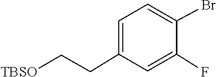 [SMILES: CC(C)(C)[Si](C)(C)OCCc1ccc(Br)c(F)c1]

To a solution of 2-(4-bromo-3-fluorophenyl)ethan-1-ol (38.0 g, 173 mmol) in DCM (210 mL) was added imidazole (17.7 g, 260 mmol) at 20° C. TBSCl (36.6 g, 242 mmol, 29.7 mL) was added to the reaction mixture at 0° C. Then the mixture was stirred at 20° C. for 3 hrs. Then the mixture was adjusted to pH=6 with 5% citric acid (180 mL), and extracted with DCM (150 mL). The organic phase was adjusted to pH=8 with NaHCO₃ and then aqueous phase was extracted with DCM (100 mL). The combined organic phase was washed with brine (150 mL), dried over Na₂SO₄, filtered and concentrated in vacuum. (4-bromo-3-fluorophenethoxy)(tert-butyl)dimethylsilane (52.0 g, 156 mmol) was obtained. ¹HNMR (400 MHz, CDCl₃-d) δ ppm 7.43 (t, J=7.72 Hz, 1H), 7.00 (dd, J=9.56, 1.87 Hz, 1H), 6.89 (dd, J=8.00, 1.87 Hz, 1H), 3.80 (t, J=6.48 Hz, 2H), 2.78 (t, J=6.48 Hz, 2H), 0.84-0.89 (m, 9H),−0.05-0.01 (m, 6H); [M+H]⁺=333.1.

### Step 3: 2,6-bis(benzyloxy)-3-(4-(2-((tert-butyldimethylsilyl)oxy)ethyl)-2-fluorophenyl)pyridine

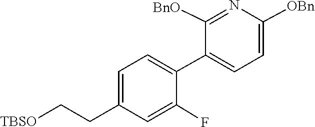 [SMILES: CC(C)(C)[Si](C)(C)OCCc1ccc(-c2ccc(OCc3ccccc3)nc2OCc2ccccc2)c(F)c1]

To a solution of (4-bromo-3-fluorophenethoxy)(tert-butyl)dimethylsilane (52.0 g, 156 mmol) and 2,6-bis(benzyloxy)-3-(4,4,5,5-tetramethyl-1,3,2-dioxaborolan-2-yl)pyridine (65.1 g, 156 mmol) in dioxane (320 mL) was added KOAc (45.9 g, 468 mmol) at 20° C. Pd(dppf)Cl₂ (11.4 g, 15.6 mmol) was added to the mixture at 20° C. The suspension was degassed under vacuum and purged with N₂ three times. Then the mixture was stirred at 90° C. for 16 hrs. Water (160 mL) was poured into the mixture, extrated with EtOAc (100 mL). The combined organic phase was washed with brine (100 mL), dried over Na₂SO₄, filtered and concentrated in vacuum. The residue was purified by column chromatography. 2,6-bis(benzyloxy)-3-(4-(2-((tert-butyldimethylsilyl)oxy)ethyl)-2-fluorophenyl)pyridine (32.0 g, 37.8%) was obtained. ¹HNMR (400 MHz, CDCl₃-d) δ ppm 7.55 (dd, J=8.04, 0.99 Hz, 1H), 7.43-7.47 (m, 2H), 7.33-7.42 (m, 7H), 7.25-7.33 (m, 3H), 6.98-7.05 (m, 2H), 5.40 (d, J=18.4 Hz, 4H), 3.87 (t, J=6.84 Hz, 1H), 3.84-3.89 (m, 1 H), 2.86 (t, J=6.84 Hz, 2H), 0.88-0.92 (m, 9H), 0.01-0.03 (m, 6H); [M+H]⁺=544.2.

### Step 4: 3-(4-(2-((tert-butyldimethylsilyl)oxy)ethyl)-2-fluorophenyl)piperidine-2,6-dione

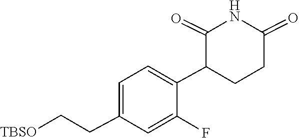 [SMILES: CC(C)(C)[Si](C)(C)OCCc1ccc(C2CCC(=O)NC2=O)c(F)c1]

To a solution of 2,6-bis(benzyloxy)-3-(4-(2-((tert-butyldimethylsilyl)oxy)ethyl)-2-fluorophenyl)pyridine (32.0 g, 58.8 mmol) in THF (50.0 mL) was added Pd/C (0.800 g, 10.0% purity) under Ar at 20° C. The suspension was degassed and purged with H₂ for 3 times. The mixture was stirred under H₂ (50 Psi) at 50° C. for 16 hrs. The suspension was filtered through a pad of celite and the filter cake was washed with THF (200 mL×3). The combined filtrates were concentrated to dryness to give crude product. The crude product was triturated with petroleum ether (50.0 mL) at 20° C. for 1 hrs. 3-(4-(2-((tert-butyldimethylsilyl)oxy)ethyl)-2-fluorophenyl)piperidine-2,6-dione (12.0 g, 55.7%) was obtained. ¹HNMR (400 MHz, CDCl₃-d) δ ppm 7.06-7.12 (m, 1H) 7.93 (br s, 1H), 6.96-7.04 (m, 2H), 3.91 (dd, J=11.2, 5.04 Hz, 1H), 3.81 (t, J=6.80 Hz, 2 H), 2.82 (t, J=6.80 Hz, 2H), 2.58-2.73 (m, 2H), 2.18-2.34 (m, 2H), 0.87 (s, 9H), 0.00 (s, 6H); [M+H]⁺=366.2.

### Step 5: 3-(2-fluoro-4-(2-hydroxyethyl)phenyl)piperidine-2,6-dione

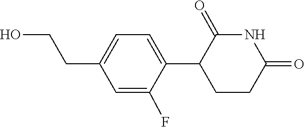 [SMILES: O=C1CCC(c2ccc(CCO)cc2F)C(=O)N1]

To a solution of 3-(4-(2-((tert-butyldimethylsilyl)oxy)ethyl)-2-fluorophenyl)piperidine-2,6-dione (12.0 g, 32.8 mmol) in MeOH (60 mL) was added HCl (12 M, 6 mL) at 20° C. Then the mixture was stirred at 20° C. for 3 hrs. Water (60 mL) was poured into the mixture, extrated with EtOAc (40 mL). The combined organic phase was washed with brine (40 mL), dried over Na₂SO₄, filtered and concentrated in vacuum. The combined crude product was purified by re-crystallization from toluene (32.0 mL) at 100° C. 3-(2-fluoro-4-(2-hydroxyethyl)phenyl)piperidine-2,6-dione (6.50 g, 78.8%) was obtained. ¹HNMR (400 MHz, DMSO-d₆) δ ppm 10.8 (s, 1H), 7.19 (t, J=7.84 Hz, 1H), 6.99-7.08 (m, 2H), 4.67 (t, J=5.18 Hz, 1H), 3.99 (dd, J=12.6, 4.74 Hz, 1H), 3.55-3.66 (m, 2H), 2.68-2.75 (m, 3H), 2.18 (qd, J=12.8, 3.86 Hz, 1H), 1.93-2.03 (m, 1H); [M+H]⁺=252.2.

### Step 6: 2-(4-(2,6-dioxopiperidin-3-yl)-3-fluorophenyl)acetaldehyde

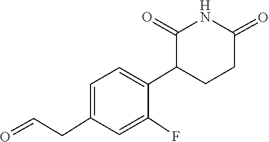 [SMILES: O=CCc1ccc(C2CCC(=O)NC2=O)c(F)c1]

To a solution of 3-(2-fluoro-4-(2-hydroxyethyl)phenyl)piperidine-2,6-dione (200 mg, 0.8 mmol) in DMSO (10 mL) was added IBX (338 mg, 1.2 mmol). The mixture was stirred in a flask at rt overnight. After being determined the reaction to be completed by LCMS, the mixture was extracted with EA (30 mL * 3). The combined organic phase was dried over anhydrous Na₂SO₄, and evaporated in vacuum to afford the crude product (100 mg, crude), which was used for next step without further purification. [M+H]⁺=250.3.

### Step 7: tert-butyl(S)-2-(hydroxymethyl)-4-(1-(5-methoxy-4-nitro-2-vinylphenyl)piperidin-4-yl)piperazine-1-carboxylate

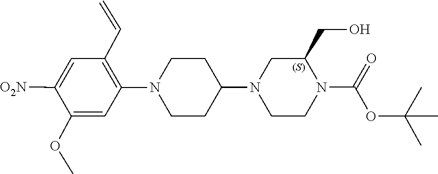

To a solution of 1-(5-methoxy-4-nitro-2-vinylphenyl)piperidin-4-one (600 mg, 2.17 mmol), tert-butyl(S)-2-(hydroxymethyl)piperazine-1-carboxylate (511.7 mg, 2.39 mmol) in DCE (10 mL) was added Ti(iPrO)₄ (27.88 mg 0.216 mmol). Then the mixture was stirred at 60° C. for 12 h and water (10 mL) was poured into the mixture. The reaction mixture was filtered and extracted with EtOAc (10 mL). The combined organic phase was washed with brine (100 mL), dried over Na₂SO₄, filtered, and concentrated in vacuum. The residue was purified by column chromatography to afford product (700 mg, 67.6%). [M+H]⁺=477.2

### Step 8: tert-butyl(S)-4-(1-(4-amino-2-ethyl-5-methoxyphenyl)piperidin-4-yl)-2-(hydroxymethyl) piperazine-1-carboxylate

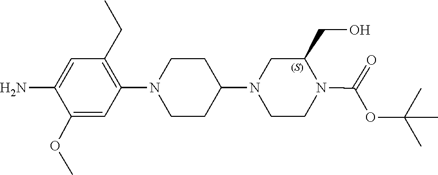

To a solution of tert-butyl(S)-2-(hydroxymethyl)-4-(1-(5-methoxy-4-nitro-2-vinylphenyl)piperidin-4-yl)piperazine-1-carboxylate (600 mg, 1.26 mmol), in DCM (10 mL), MeOH (10 mL) was added Pd—C under N₂ atmosphere. The suspension was degassed under vacuum and purged with H₂ several times. The mixture was stirred under H₂ balloon at rt for 12 h. Then the mixture was stirred at 60° C. for another 12 h. The reaction mixture was filtered and extracted with DCM (3×30 mL). The combined organic phase was washed with brine (100 mL), dried over Na₂SO₄, filtered, and concentrated in vacuum. The residue was purified by column chromatography (DCM/MeOH=10/1 to 5/1) to afford product (500 mg, 88.5%). [M+H]⁺=449.1.

### Step 9: (S)-(6-((5-bromo-2-((5-ethyl-4-(4-(3-(hydroxymethyl)piperazin-1-yl)piperidin-1-yl)-2-methoxyphenyl)amino)pyrimidin-4-yl)amino)quinoxalin-5-yl)dimethylphosphine oxide

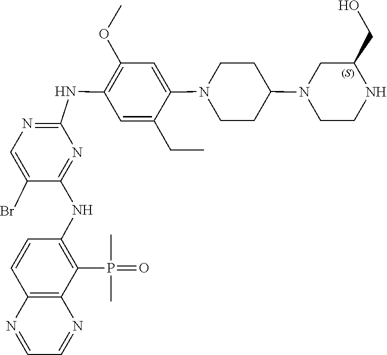

To a solution of tert-butyl(S)-4-(1-(4-amino-2-ethyl-5-methoxyphenyl)piperidin-4-yl)-2-(hydroxymethyl)piperazine-1-carboxylate (300 mg, 0.589 mmol) and (6-((5-bromo-2-chloropyrimidin-4-yl)amino)quinoxalin-5-yl)dimethylphosphine oxide (267.9 mg, 0.648 mmol) in n-BuOH(10 mL) was added TsOH (336.12 mg 1.767 mmol). Then the mixture was stirred at 100° C. for 12 h. Water (30 mL) was poured into the mixture. The reaction mixture was filtered and extracted with DCM (2×50 mL). The combined organic phase was washed with brine (2×100 mL), dried over Na₂SO₄, filtered, and concentrated in vacuum. The residue was purified by column chromatography to afford product (230 mg, 47.5%). [M+H]⁺=724.2.

### Step 10: 3-(4-(2-((S)-4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)-2-(hydroxymethyl)piperazin-1-yl)ethyl)-2-fluorophenyl)piperidine-2,6-dione

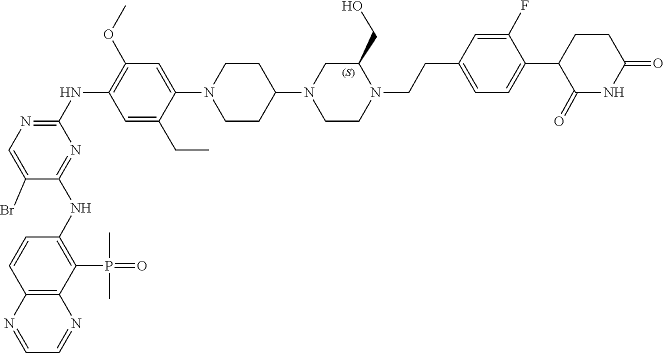

The titled compound (11 mg, 26%) was prepared in a manner similar to that in Example 204 step 6 from (S)-(6-((5-bromo-2-((5-ethyl-4-(4-(3-(hydroxymethyl)piperazin-1-yl)piperidin-1-yl)-2-methoxyphenyl)amino)pyrimidin-4-yl)amino)quinoxalin-5-yl)dimethylphosphine oxide and 2-(4-(2,6-dioxopiperidin-3-yl)-3-fluorophenyl)acetaldehyde. 1H NMR (500 MHz, DMSO) δ 12.65 (s, 1H), 10.86 (s, 1H), 8.85 (t, J=16.5 Hz, 3H), 8.27 (s, 2H), 7.90 (d, J=8.2 Hz, 1H), 7.38 (s, 1H), 7.20 (d, J=7.8 Hz, 1H), 7.06 (d, J=21.3 Hz, 2H), 6.81 (s, 1H), 4.00 (d, J=17.4 Hz, 1H), 3.77 (s, 3H), 3.64 (s, 1H), 3.41 (s, 2H), 3.01 (s, 2H), 2.88 (d, J=6.4 Hz, 3H), 2.63 (s, 8H), 2.38 (d, J=42.8 Hz, 6H), 2.19 (s, 2H), 2.02 (d, J=14.4 Hz, 7H), 1.85 (s, 2H), 1.59 (d, J=11.6 Hz, 2H), 0.94 (s, 3H). [M+H]+=957.6.

### Example 6: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-3-fluorophenyl)piperidine-2,6-dione

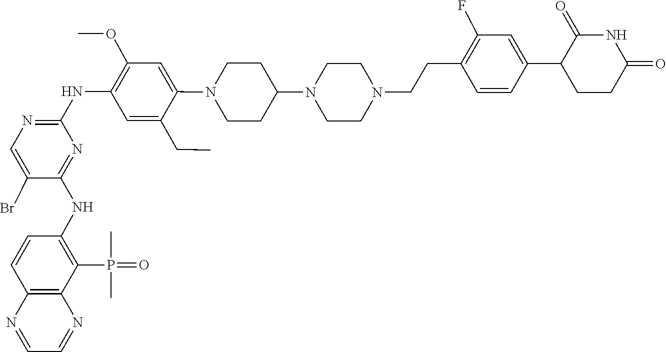 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)cc3F)CC2)CC1]

The titled compound was synthesized in a manner similar to that in Example 200. 1H NMR (400 MHz, DMSO) δ 12.64 (s, 1H), 10.86 (s, 1H), 8.85 (t, J=15.3 Hz, 3H), 8.29 (d, J=13.8 Hz, 2H), 7.91 (s, 1H), 7.37 (s, 1H), 7.29 (t, J=8.0 Hz, 1H), 7.04 (d, J=11.2 Hz, 1H), 6.99 (d, J=8.0 Hz, 1H), 6.81 (s, 1H), 3.86 (dd, J=12.0, 4.8 Hz, 1H), 3.77 (s, 3H), 3.01 (d, J=10.7 Hz, 2H), 2.73-2.68 (m, 7H), 2.56 (s, 3H), 2.52 (s, 1H), 2.48 (s, 5H), 2.6-2.32 (m, 2H), 2.25-2.21 (m, 1H), 2.02 (d, J=14.4 Hz, 8H), 1.87 (d, J=10.0 Hz, 2H), 1.58 (d, J=9.3 Hz, 2H), 0.92 (t, J=7.2 Hz, 3H); [M+H]⁺=927.6.

### Example 10: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2,3-difluorophenyl)piperidine-2,6-dione

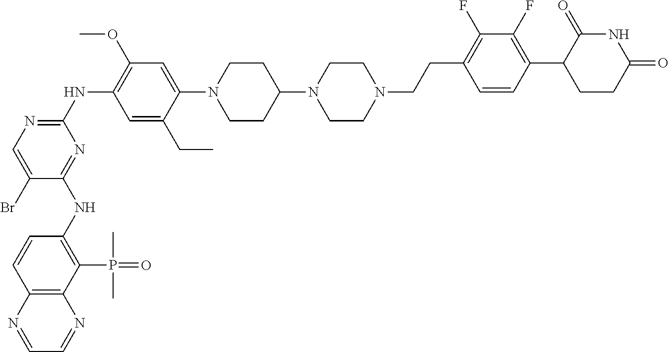 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)c(F)c3F)CC2)CC1]

The titled compound was synthesized in a manner similar to that in Example 200. 1H NMR (400 MHz, DMSO) δ 12.65 (s, 1H), 10.94 (s, 1H), 8.86 (d, J=20.7, 10.3 Hz, 3H), 8.28 (d, J=8.5 Hz, 2H), 7.90 (d, J=8.8 Hz, 1H), 7.37 (s, 1H), 7.14 (t, J=7.1 Hz, 1H), 7.06 (t, J=7.0 Hz, 1H), 6.81 (s, 1H), 4.10 (dd, J=12.7, 4.9 Hz, 1H), 3.77 (s, 3H), 3.01 (d, J=11.1 Hz, 2H), 2.83-2.64 (m, 6H), 2.55 (dd, J=16.4, 5.5 Hz, 6H), 2.47 (d, J=7.2 Hz, 5H), 2.34 (d, J=21.6 Hz, 2H), 2.23 (dd, J=12.9, 4.0 Hz, 1H), 2.02 (d, J=14.4 Hz, 7H), 1.86 (d, J=11.5 Hz, 2H), 1.62-1.54 (m, 2H), 0.93 (t, J=6.6 Hz, 3H); [M+H]⁺=945.6.

### Example 11 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

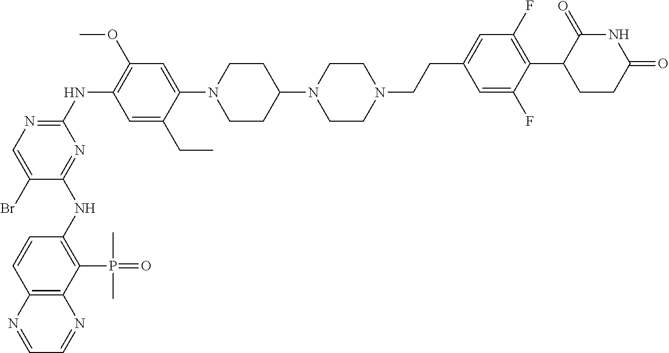 [SMILES: CCc1cc(Nc2ncc(Br)c(Nc3ccc4nccnc4c3P(C)(C)=O)n2)c(OC)cc1N1CCC(N2CCN(CCc3cc(F)c(C4CCC(=O)NC4=O)c(F)c3)CC2)CC1]

The titled compound was synthesized in a manner similar to that in Example 200.

1H NMR (400 MHz, DMSO) δ 12.65 (s, 1H), 10.96 (s, 1H), 8.86 (dt, J=22.8, 11.4 Hz, 3H), 8.28 (d, J=9.1 Hz, 2H), 7.90 (d, J=8.9 Hz, 1H), 7.37 (s, 1H), 7.03 (d, J=10.0 Hz, 2H), 6.81 (s, 1H), 4.20 (dd, J=12.8, 5.0 Hz, 1H), 3.77 (s, 3H), 3.01 (d, J=11.5 Hz, 2H), 2.76 (m, 6H), 2.54 (d, J=1.8 Hz, 6H), 2.48 (s, 5H), 2.36 (s, 2H), 2.13 (d, J=9.6 Hz, 1H), 2.02 (d, J=14.4 Hz, 7H), 1.87 (d, J=11.4 Hz, 2H), 1.58 (d, J=8.8 Hz, 2H), 0.93 (t, J=7.2 Hz, 3H); [M+H]⁺=945.6.

### Example 34: 5-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)piperidin-1-yl)-2-(2,6-dioxopiperidin-3-yl)benzonitrile

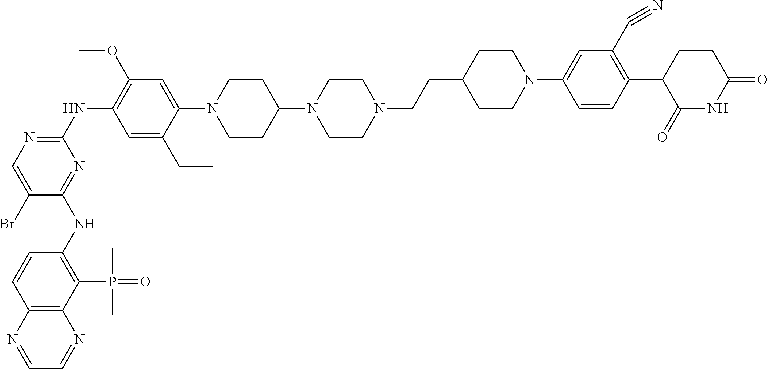 [SMILES: CCc1cc(Nc2ncc(Br)c(Nc3ccc4nccnc4c3P(C)(C)=O)n2)c(OC)cc1N1CCC(N2CCN(CCC3CCN(c4ccc(C5CCC(=O)NC5=O)c(C#N)c4)CC3)CC2)CC1]

The titled compound was synthesized in a manner similar to that in Example 200.

1H NMR (400 MHz, DMSO) δ 12.64 (s, 1H), 10.93 (s, 1H), 8.86 (d, J=4.5 Hz, 2H), 8.84-8.79 (m, 1H), 8.27 (s, 2H), 7.91 (s, 1H), 7.37 (s, 1H), 7.29 (s, 1H), 7.23 (s, 2H), 6.81 (s, 1H), 3.99 (d, J=9.2 Hz, 2H), 3.77 (s, 7H), 3.00 (s, 2H), 2.70 (t, J=11.0 Hz, 5H), 2.52 (s, 5H), 2.48-2.45 (m, 1H), 2.31 (s, 6H), 2.02 (d, J=14.4 Hz, 8H), 1.84 (s, 2H), 1.74 (d, J=12.1 Hz, 2H), 1.58 (s, 2H), 1.40 (s, 2H), 1.23 (s, 2H), 0.93 (t, J=7.2 Hz, 3H); [M+H]⁺=1017.4.

### Example 208: 3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

### Step 1: methyl (R)-1-(4-(2,6-bis(benzyloxy)pyridin-3-yl)-3,5-difluorophenyl)pyrrolidine-3-carboxylate

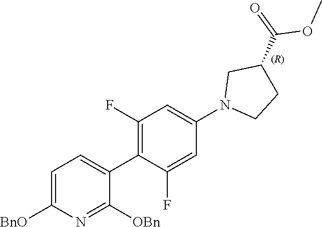

To a solution of 2,6-bis(benzyloxy)-3-(4-bromo-2,6-difluorophenyl)pyridine (1 g, 2.07 mmol), methyl (R)-pyrrolidine-3-carboxylate hydrochloride (495 mg, 3 mmol) and Cs₂CO₃ (1.95 g, 6 mmol) in 10 mL DMSO, Pd₂(dba)₃ (183 mg, 0.2 mmol) and Xantphos (231 mg, 0.4 mmol) was added under N₂ atmosphere. The mixture was stirred at 90° C. for 16 hours under N₂ atmosphere. After LCMS showed the reaction was completed. The mixture was diluted with EtOAc (100 mL) and washed with brine (100 mL×2). The organic phase was dried over Na₂SO₄, filtered and concentrated in vacuum. The residue was purified by silica column chromatography (PE:EA=10:1) to afford product (740 mg, 67.4% yield). [M+H]⁺=530.8.

### Step 2: (R)-1-(4-(2,6-bis(benzyloxy)pyridin-3-yl)-3,5-difluorophenyl)pyrrolidine-3-carboxylic acid

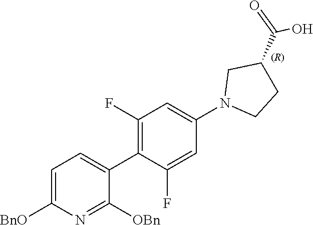

To a solution of methyl (R)-1-(4-(2,6-bis(benzyloxy)pyridin-3-yl)-3,5-difluorophenyl)pyrrolidine-3-carboxylate (740 mg, 1.4 mmol) in 10 mL THF, LiOH H₂O (84 mg, 2 mmol) in 2 mL water was added. The mixture was stirred at 25° C. for 2 hours. After LCMS showed the reaction was completed. The mixture was concentrated in vacuum. The residue was adjust pH<5 with 1 N HCl and extracted with 50 mL EtOAc. The organic phase was dried over Na₂SO₄, filtered and concentrated in vacuum to afford product (700 mg, 96.9% yield). [M+H]⁺=516.8.

### Step 3: (3R)-1-(4-(2,6-dioxopiperidin-3-yl)-3,5-difluorophenyl)pyrrolidine-3-carboxylic acid

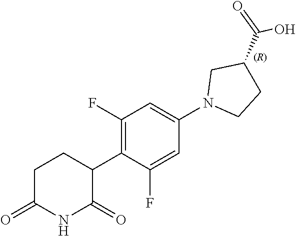

To a solution of (R)-1-(4-(2,6-bis(benzyloxy)pyridin-3-yl)-3,5-difluorophenyl)pyrrolidine-3-carboxylic acid (700 mg, 1.35 mmol) in 5 mL DCM and 30 mL MeOH, 350 mg Pd/C was added. The mixture was stirred at 30° C. for 16 hours under H₂ atmosphere. After LCMS showed the reaction was completed and the mixture was filtered. The organic phase was concentrated in vacuum to afford product (350 mg, 76.7% yield). [M+H]⁺=338.8.

### Step 4: 5-iodo-2-methylquinolin-6-amine

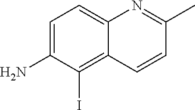

To a solution of 2-methylquinolin-6-amine (5 g, 31.62 mmol) in AcOH(50 mL) was added dropwise a solution of ICl (8.85 g, 37.95 mmol) in AcOH(10 mL) was stirred at 5° C.˜10° C. Then the mixture was stirred at rt for 1h. The reaction solution was concentrated to dryness and the mixture was diluted with water (200 mL), neutralized with solid K₂CO₃. The mixture was extracted with DCM (3×150 mL). The combined organic phase was washed with brine (2×100 mL), dried over Na₂SO₄, filtered, and concentrated in vacuum. The residue was purified by column chromatography (DCM:MeOH=20:1) to afford product (7.1 g, 79.06%). [M+H]⁺=285.2.

### Step 5: (6-amino-2-methylquinolin-5-yl)dimethylphosphine oxide

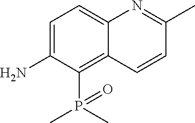 [SMILES: Cc1ccc2c(P(C)(C)=O)c(N)ccc2n1]

To a solution of 5-iodo-2-methylquinolin-6-amine (7.1 g, 24.99 mmol) and dimethylphosphine oxide (2.93 g, 37.49 mmol) in dioxane (100 mL) was added Pd(OAc)₂ (0.55 g, 2.45 mmol), Xantphos (2.89 g, 4.99 mmol), K₃PO₄ (10.61 g, 49.98 mmol) under N₂ atmosphere. The mixture was degassed under vacuum and purged with N₂ several times. The mixture was stirred under N₂ balloon at 100° C. for 6 h. The reaction mixture was extracted with DCM (3×50 mL). The combined organic phase was washed with brine (100 mL), dried over Na₂SO₄ and concentrated in vacuum. The residue was purified by column chromatography (DCM:MeOH=15:1) to afford the product (4.5 g, 76.9%). [M+H]⁺=235.2.

### Step 6: (6-((5-bromo-2-chloropyrimidin-4-yl)amino)-2-methylquinolin-5-yl)dimethylphosphine oxide

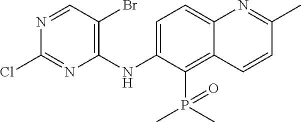 [SMILES: Cc1ccc2c(P(C)(C)=O)c(Nc3nc(Cl)ncc3Br)ccc2n1]

A solution of (6-amino-2-methylquinolin-5-yl)dimethylphosphine oxide (4.5 g, 19.21 mmol), 5-bromo-2,4-dichloropyrimidine (13.13 g, 57.64 mmol) and DIEA (7.45 g, 57.64 mmol) in n-BuOH (100 mL) was stirred at 120° C. for 12 h. The reaction solution was concentrated to dryness, then the crude product was purified by re-crystallization from EA:PE=5:1 (50 mL). The mixture was filtered and the filter cake was washed with DCM (3×50 mL). The combined organic phase was washed with brine (2×100 mL), dried over Na₂SO₄ and concentrated in vacuum to afford product (5.5 g, 67.3%). [M+H]⁺=425.2.

### Step 7

### (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide

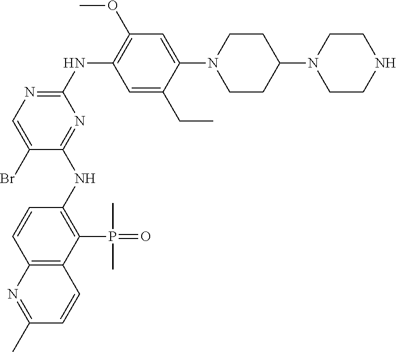 [SMILES: CCc1cc(Nc2ncc(Br)c(Nc3ccc4nc(C)ccc4c3P(C)(C)=O)n2)c(OC)cc1N1CCC(N2CCNCC2)CC1]

A solution of (6-((5-bromo-2-chloropyrimidin-4-yl)amino)-2-methylquinolin-5-yl)dimethylphosphine oxide (5.5 g, 12.91 mmol), TsOH (6.67 g, 38.73 mmol) and tert-butyl 4-(1-(4-amino-2-ethyl-5-methoxyphenyl)piperidin-4-yl)piperazine-1-carboxylate (5.67 g, 13.56 mmol) in n-BuOH (80 mL) was stirred at 100° C. for 12 h. The reaction mixture was adjusted to pH=8 with 1M NaOH, and then extracted with DCM (3×80 mL). The combined organic phase was washed with brine (2×100 mL), dried over Na₂SO₄ and concentrated in vacuum, The residue was purified by column chromatograph (DCM:MeOH=8:1) to afford the product (5.2 g, 57.01%). [M+H]⁺=707.3.

### Step 8: 3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

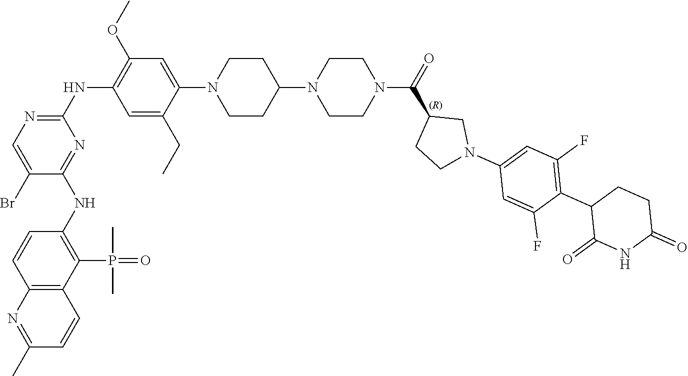

To a solution of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (50 mg, 0.07 mmol), (3R)-1-(4-(2,6-dioxopiperidin-3-yl)-3,5-difluorophenyl)pyrrolidine-3-carboxylic acid (24 mg, 0.07 mmol) and DIEA (26 mg, 0.2 mmol) in 10 mL DCM, 50% w.t. T₃P in EtOAc solution (64 mg, 0.1 mmol) was added. The mixture was stirred at 25° C. for 16 hours. After LCMS showed the reaction was completed. The mixture was quenched with 10 mL water. The organic phase was concentrated in vacuum and purified by prep-HPLC with C-18 column chromatography (0.1% FA in water: acetonitrile=90: 10˜ 60: 40 gradient elution) to afford the desired product (21.69 mg, 30.1% yield). ¹H NMR (500 MHz, DMSO) δ 11.76 (s, 1H), 10.84 (s, 1H), 8.56 (d, J=8.9 Hz, 1H), 8.30 (d, J=7.1 Hz, 1H), 8.21 (s, 1H), 7.98 (s, 1H), 7.87 (d, J=9.2 Hz, 1H), 7.46-7.33 (m, 2H), 6.74 (s, 1H), 6.23 (d, J=12.1 Hz, 2H), 4.02 (dd, J=12.6, 5.1 Hz, 1H), 3.76 (s, 3H), 3.59-3.42 (m, 6H), 3.36-3.22 (m, 4H), 2.95 (d, J=10.7 Hz, 2H), 2.83-2.73 (m, 1H), 2.70-2.62 (m, 5H), 2.56 (d, J=15.8 Hz, 2H), 2.45-2.35 (m, 3H), 2.30 (d, J=7.1 Hz, 2H), 2.21-2.03 (m, 3H), 2.03-1.91 (m, 7H), 1.84 (d, J=10.2 Hz, 2H), 1.65-1.50 (m, 2H), 0.92-0.63 (m, 3H). [M+H]⁺=1027.6.

### Example 64: 5-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)-2-(2,6-dioxopiperidin-3-yl)isoindoline-1,3-dione

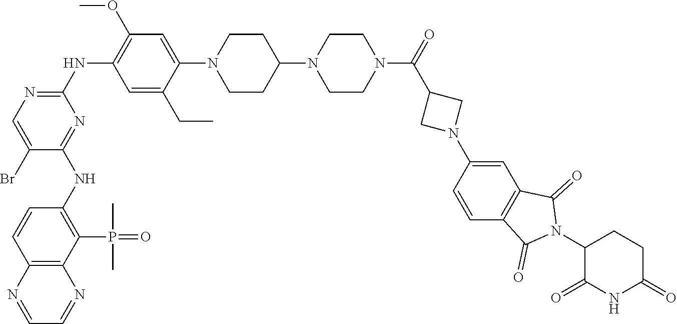 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc5c(c4)C(=O)N(C4CCC(=O)NC4=O)C5=O)C3)CC2)CC1]

The titled compound was prepared in a manner similar to that in Example 57. ¹H NMR (400 MHz, DMSO) δ 12.64 (s, 1H), 11.08 (s, 1H), 8.85 (t, J=9.5 Hz, 3H), 8.28 (d, J=6.1 Hz, 2H), 7.90 (d, J=8.6 Hz, 1H), 7.66 (d, J=8.1 Hz, 1H), 7.38 (s, 1H), 6.83 (d, J=11.6 Hz, 2H), 6.71 (d, J=8.3 Hz, 1H), 5.07 (d, J=12.8 Hz, 1H), 4.23 (d, J=8.4 Hz, 2H), 4.14 (s, 2H), 3.92 (s, 2H), 3.77 (s, 4H), 3.52 (s, 3H), 3.03 (d, J=10.2 Hz, 3H), 2.87 (d, J=13.4 Hz, 2H), 2.72 (t, J=10.5 Hz, 3H), 2.57 (s, 2H), 2.37 (d, J=34.2 Hz, 2H), 2.02 (d, J=14.4 Hz, 8H), 1.85 (s, 2H), 1.61 (d, J=9.8 Hz, 2H), 0.93 (s, 3H). [M+H]⁺=1035.9

### Example 65: 5-((R)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)pyrrolidin-1-yl)-2-(2,6-dioxopiperidin-3-yl)isoindoline-1,3-dione

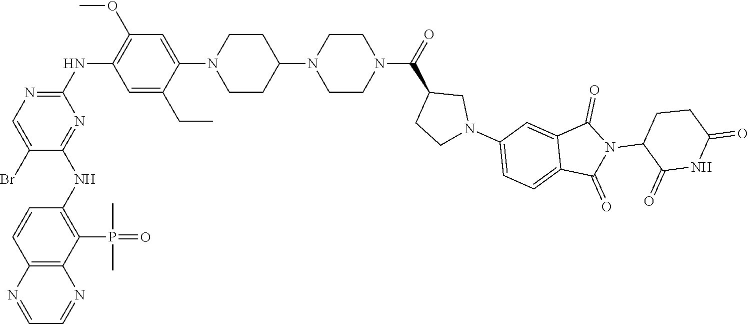 [SMILES: CCc1cc(Nc2ncc(Br)c(Nc3ccc4nccnc4c3P(C)(C)=O)n2)c(OC)cc1N1CCC(N2CCN(C(=O)[C@@H]3CCN(c4ccc5c(c4)C(=O)N(C4CCC(=O)NC4=O)C5=O)C3)CC2)CC1]

The titled compound was prepared in a manner similar to that in Example 57. ¹H NMR (400 MHz, DMSO) δ 12.65 (s, 1H), 11.08 (s, 1H), 8.87 (d, J=4.8 Hz, 3H), 8.27 (t, J=11.6 Hz, 2H), 7.90 (d, J=8.9 Hz, 1H), 7.66 (d, J=8.3 Hz, 1H), 7.38 (s, 1H), 6.98-6.79 (m, 3H), 5.06 (d, J=8.0 Hz, 1H), 3.77 (s, 3H), 3.69-3.48 (m, 10H), 3.02 (s, 3H), 2.96-2.81 (m, 2H), 2.72 (t, J=10.7 Hz, 2H), 2.60 (s, 4H), 2.42 (s, 1H), 2.24 (s, 1H), 2.14 (s, 1H), 2.04 (t, J=14.1 Hz, 8H), 1.86 (s, 2H), 1.62 (d, J=10.0 Hz, 2H), 0.93 (s, 3H). [M+H]⁺=1049.8

### Example 66: 5-((S)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)pyrrolidin-1-yl)-2-(2,6-dioxopiperidin-3-yl)isoindoline-1,3-dione

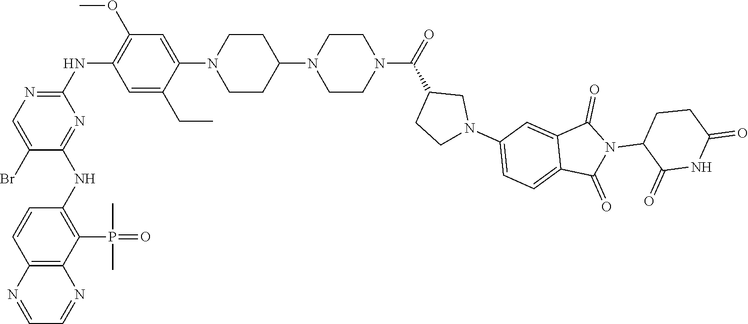

The titled compound was prepared in a manner similar to that in Example 57. ¹H NMR (400 MHz, DMSO) δ 12.65 (s, 1H), 11.08 (s, 1H), 8.87 (d, J=4.4 Hz, 3H), 8.28 (d, J=6.9 Hz, 2H), 7.89 (s, 1H), 7.66 (d, J=8.5 Hz, 1H), 7.38 (s, 1H), 6.94 (s, 1H), 6.82 (s, 2H), 5.07 (s, 1H), 3.77 (s, 3H), 3.55 (d, J=32.6 Hz, 10H), 3.02 (s, 3H), 2.88 (s, 2H), 2.70 (d, J=21.8 Hz, 3H), 2.60 (s, 3H), 2.45-2.37 (m, 1H), 2.23 (s, 1H), 2.14 (s, 1H), 2.02 (d, J=14.2 Hz, 7H), 1.86 (s, 2H), 1.63 (s, 2H), 0.93 (s, 3H). [M+H]⁺=1049.8

### Example 67: 5-(4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)piperidin-1-yl)-2-(2,6-dioxopiperidin-3-yl)isoindoline-1,3-dione

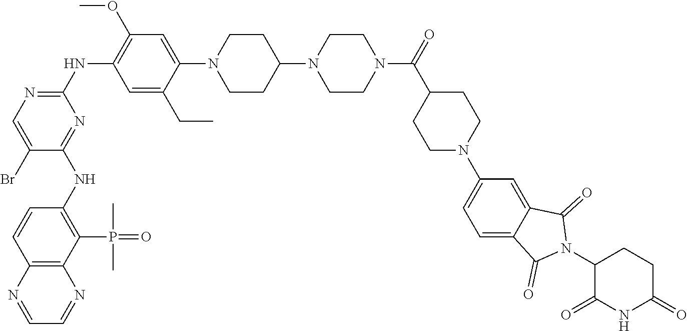 [SMILES: CCc1cc(Nc2ncc(Br)c(Nc3ccc4nccnc4c3P(C)(C)=O)n2)c(OC)cc1N1CCC(N2CCN(C(=O)C3CCN(c4ccc5c(c4)C(=O)N(C4CCC(=O)NC4=O)C5=O)CC3)CC2)CC1]

The titled compound was prepared in a manner similar to that in Example 57. ¹H NMR (400 MHz, DMSO) δ 12.64 (s, 1H), 11.08 (s, 1H), 8.86 (s, 3H), 8.27 (s, 2H), 7.91 (s, 1H), 7.66 (s, 1H), 7.43-7.23 (m, 3H), 6.81 (s, 1H), 5.06 (s, 1H), 4.07 (d, J=10.2 Hz, 2H), 3.77 (s, 3H), 3.56 (s, 2H), 3.46 (s, 4H), 3.12-2.95 (m, 8H), 2.92-2.83 (m, 2H), 2.72 (s, 2H), 2.32 (s, 2H), 2.02 (d, J=14.1 Hz, 8H), 1.85 (s, 2H), 1.66 (d, J=28.9 Hz, 6H), 0.93 (s, 3H). [M+H]⁺=1061.3.

### Example 70: 3-(5-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)piperidin-1-yl)-1-oxoisoindolin-2-yl)piperidine-2,6-dione

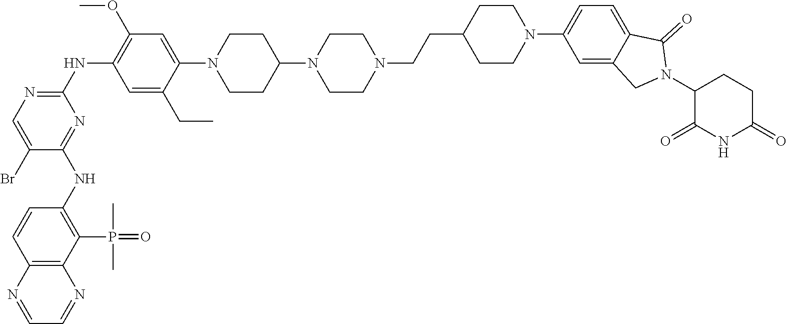 [SMILES: CCc1cc(Nc2ncc(Br)c(Nc3ccc4nccnc4c3P(C)(C)=O)n2)c(OC)cc1N1CCC(N2CCN(CCC3CCN(c4ccc5c(c4)CN(C4CCC(=O)NC4=O)C5=O)CC3)CC2)CC1]

The titled compound was synthesized in the procedures similar to Example 237. ¹H NMR (400 MHz, DMSO) δ 12.55 (s, 1H), 10.95 (s, 1H), 8.84 (t, J=13.9 Hz, 3H), 8.24 (d, J=22.1 Hz, 3H), 7.91 (d, J=8.6 Hz, 1H), 7.52 (d, J=9.0 Hz, 1H), 7.38 (s, 1H), 7.06 (s, 2H), 6.80 (s, 1H), 5.02 (d, J=7.9 Hz, 1H), 4.32 (s, 1H), 4.21 (d, J=17.1 Hz, 1H), 3.86 (d, J=12.6 Hz, 6H), 3.02 (d, J=10.3 Hz, 3H), 2.89 (s, 2H), 2.83 (d, J=12.6 Hz, 2H), 2.69 (d, J=11.9 Hz, 3H), 2.63 (s, 4H), 2.42 (s, 2H), 2.06 (d, J=7.7 Hz, 9H), 2.01 (s, 3H), 1.88 (s, 2H), 1.75 (d, J=12.4 Hz, 2H), 1.58 (d, J=10.9 Hz, 2H), 1.43 (s, 2H), 1.23 (s, 3H), 0.91 (s, 3H). [M+H]⁺=1050.1.

### Example 71: 3-(5-((2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)(methyl)amino)-1-oxoisoindolin-2-yl)piperidine-2,6-dione Step 1: methyl 2-cyano-4-((2-hydroxyethyl)(methyl)amino)benzoate

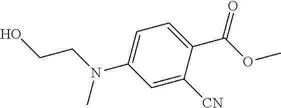 [SMILES: COC(=O)c1ccc(N(C)CCO)cc1C#N]

A mixture of methyl 2-cyano-4-fluorobenzoate (10.00 g, 55.87 mmol) and 2-(methylamino)ethan-1-ol (4.61 g, 61.45 mmol), DIEA (23.78 g, 184.35 mmol) in DMSO (100 mL) was stirred for 3 h at 60° C. The resulting mixture was extracted with EtOAc. The combined organic layers were washed with brine, dried over anhydrous Na₂SO₄. After filtration, the filtrate was concentrated under reduced pressure to afford the product (10 g, 76.51%). m/z[M+H]⁺=235.1.

### Step 2: methyl 2-formyl-4-((2-hydroxyethyl)(methyl)amino)benzoate

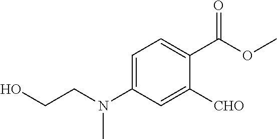 [SMILES: COC(=O)c1ccc(N(C)CCO)cc1C=O]

To a stirred mixture of methyl 2-cyano-4-((2-hydroxyethyl)(methyl)amino)benzoate (10.0 g, 42.74 mmol) and oxophosphinic acid sodium hydride (44.44 g, 427.35 mmol, 60%) in pyridine (100.00 mL), AcOH (40.00 mL) and H₂O (40.00 mL) was added Raney-Ni (10.00 g) in portions and stirred at 70° C. under nitrogen atmosphere for 3 days. The reaction was quenched with 1 M HCl at room temperature. The solid was filtered out. The filtrate was concentrated under vacuum. The resulting mixture was extracted with EtOAc. The combined organic layers were washed with citric acid, dried over anhydrous Na₂SO₄. After filtration, the filtrate was concentrated under reduced pressure. The residue was purified by silica gel column chromatography, eluted with CH₂Cl₂/MeOH (10:1) to afford the product (5 g, 49.4%). [M+H]⁺=238.1.

### Step 3: 3-(5-((2-hydroxyethyl)(methyl)amino)-1-oxoisoindolin-2-yl)piperidine-2,6-dione

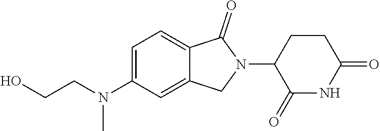 [SMILES: CN(CCO)c1ccc2c(c1)CN(C1CCC(=O)NC1=O)C2=O]

A mixture of 3-aminopiperidine-2,6-dione (5.82 g, 42.19 mmol) and DIEA (13.71 g, 105.49 mmol) in DMF (50.00 mL) was stirred for 5 h at room temperature. The mixture was acidified to pH=6 with AcOH (7.6 g, 126.58 mmol). To the above mixture was added methyl 2-formyl-4-((2-hydroxyethyl)(methyl)amino)benzoate (5.0 g, 21.1 mmol) at room temperature. The resulting mixture was stirred overnight at room temperature. To the above mixture was added NaBH₃CN (5.23 g, 84.39 mmol) in portions at room temperature. The resulting mixture was stirred overnight at room temperature. The reaction was quenched with water at room temperature. The resulting mixture was concentrated under vacuum. The residue was purified by silica gel column chromatography, eluted with CH₂Cl₂/MeOH (8:1) to afford the product (2.61 g, 39.0%). [M+H]⁺=318.3.

### Step 4: 2-((2-(2,6-dioxopiperidin-3-yl)-1-oxoisoindolin-5-yl)(methyl)amino)ethyl methanesulfonate

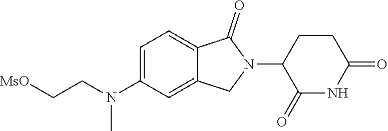 [SMILES: CN(CCOS(C)(=O)=O)c1ccc2c(c1)CN(C1CCC(=O)NC1=O)C2=O]

The titled compound (150 mg, 76%) was prepared in a manner similar to that in Example 207 step 4 from 3-(5-((2-hydroxyethyl)(methyl)amino)-1-oxoisoindolin-2-yl)piperidine-2,6-dione and MsCl. [M+H]⁺=395.1.

### Step 5: 3-(5-((2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)(methyl)amino)-1-oxoisoindolin-2-yl)piperidine-2,6-dione

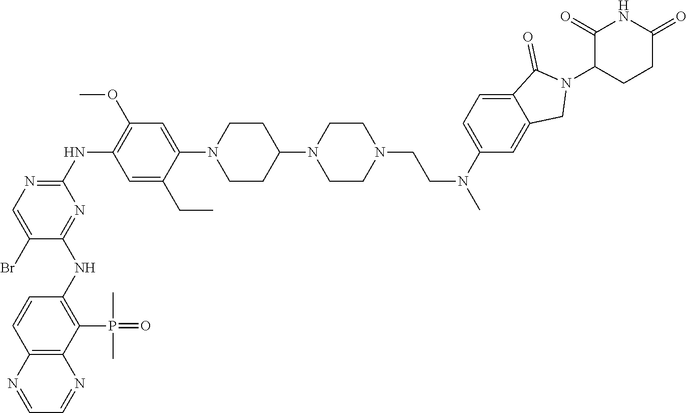 [SMILES: CCc1cc(Nc2ncc(Br)c(Nc3ccc4nccnc4c3P(C)(C)=O)n2)c(OC)cc1N1CCC(N2CCN(CCN(C)c3ccc4c(c3)CN(C3CCC(=O)NC3=O)C4=O)CC2)CC1]

The titled compound (12 mg, 26%) was prepared in a manner similar to that in Example 207 step 5 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 2-((2-(2,6-dioxopiperidin-3-yl)-1-oxoisoindolin-5-yl)(methyl)amino)ethyl methanesulfonate. ¹H NMR (400 MHz, DMSO) δ 12.64 (s, 1H), 10.94 (s, 1H), 8.86 (d, J=4.9 Hz, 3H), 8.28 (s, 1H), 7.91 (s, 1H), 7.49 (d, J=8.8 Hz, 1H), 7.37 (s, 1H), 6.81 (s, 3H), 6.60 (s, 1H), 5.03 (d, J=8.5 Hz, 1H), 4.32 (d, J=16.7 Hz, 1H), 4.19 (d, J=16.8 Hz, 1H), 3.77 (s, 3H), 3.55 (s, 5H), 2.95 (d, J=43.9 Hz, 10H), 2.76-2.66 (m, 3H), 2.60 (s, 1H), 2.33 (s, 4H), 2.02 (d, J=14.4 Hz, 8H), 1.85 (s, 2H), 1.57 (d, J=9.0 Hz, 2H), 1.24 (s, 1H), 0.92 (s, 3H). [M+H]⁺=993.9.

### Example 101: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethoxy)-1-oxoisoindolin-2-yl)piperidine-2,6-dione

### Step 1: methyl 2-formyl-3-hydroxybenzoate

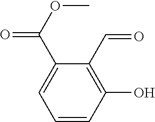

A solution of methyl 3-hydroxybenzoate (40.00 g, 262.90 mmol) in THF (1.00 L) was stirred at room temperature. To the mixture was added hexamethylenetetramine (44.17 g, 315.48 mmol) in portions at room temperature. The resulting mixture was stirred for additional 4 h at 80° C. The resulting mixture was extracted with EtOAc. The combined organic layers were washed with ice water (50 mL), dried over anhydrous Na₂SO₄. After filtration, the filtrate was concentrated under reduced pressure. The residue was purified by silica gel column chromatography, eluted with PE/EtOAc (10:1) to afford the product (20 g, 42%). [M+H]⁺=181.2.

### Step 2: methyl 3-(2-(benzyloxy)ethoxy)-2-formylbenzoate

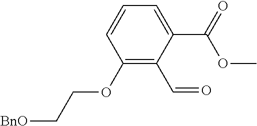 [SMILES: COC(=O)c1cccc(OCCOCc2ccccc2)c1C=O]

A mixture of methyl 2-formyl-3-hydroxybenzoate (20.00 g, 111.01 mmol) and [(2-bromoethoxy)methyl]benzene (71.63 g, 333.03 mmol), K₂CO₃ (30.69 g, 222.02 mmol), KI (9.21 g, 55.50 mmol) in DMF (300 mL) was stirred overnight at 70° C. The resulting mixture was extracted with EtOAc. The combined organic layers were washed with brine, dried over anhydrous Na₂SO₄. After filtration, the filtrate was concentrated under reduced pressure. The residue was purified by silica gel column chromatography, eluted with PE/EtOAc (2:1) to afford the product (10 g, 29%). [M+H]⁺=315.2.

### Step 3: 3-(4-(2-(benzyloxy)ethoxy)-1-oxoisoindolin-2-yl)piperidine-2,6-dione

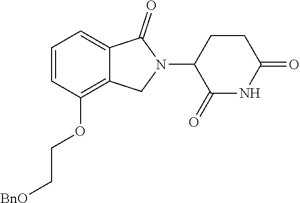 [SMILES: O=C1CCC(N2Cc3c(OCCOCc4ccccc4)cccc3C2=O)C(=O)N1]

A mixture of 3-aminopiperidine-2,6-dione hydrochloride (6.26 g, 38.17 mmol) and DIEA (8.22 g, 63.62 mmol) in DCE (100 mL) and DMF (5 mL) was stirred for 2 h at room temperature. The mixture was acidified to pH=6 with AcOH. To the above mixture was added methyl 3-(2-(benzyloxy)ethoxy)-2-formylbenzoate (10.00 g, 31.81 mmol). The resulting mixture was stirred overnight at room temperature. To the above mixture was added NaBH₃CN (6.00 g, 95.43 mmol) in portions at room temperature. The resulting mixture was stirred for additional 5 h at room temperature. The reaction was quenched by the addition of water. The resulting mixture was extracted with CH₂Cl₂. The combined organic layers were washed with brine, dried over anhydrous Na₂SO₄. After filtration, the filtrate was concentrated under reduced pressure. The residue was purified by silica gel column chromatography, eluted with CH₂Cl₂/MeOH (5:1) to afford crude product. The residue was purified by reverse phase flash chromatography with the following conditions: mobile phase, MeCN/H₂O (0 to 50% in 30 min elution gradient) to afford the product (4.1 g, 33%). [M+H]⁺=395.2.

### Step 4: 3-(4-(2-hydroxyethoxy)-1-oxoisoindolin-2-yl)piperidine-2,6-dione

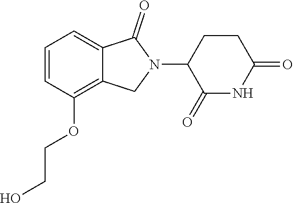 [SMILES: O=C1CCC(N2Cc3c(OCCO)cccc3C2=O)C(=O)N1]

A mixture of 3-(4-(2-(benzyloxy)ethoxy)-1-oxoisoindolin-2-yl)piperidine-2,6-dione (4.10 g, 10.39 mmol), Pd/C (2.00 g, 10% wt), AcOH (4 mL), THF (30 mL) and DCM (30 mL) was stirred for 16 h at 40° C. under hydrogen atmosphere. The resulting mixture was filtered, the filter cake was washed with MeOH and DCM. The filtrate was concentrated under reduced pressure to afford the product (2.13 g, 67%). [M+1]⁺=305.2.

### Step 5: 2-((2-(2,6-dioxopiperidin-3-yl)-1-oxoisoindolin-4-yl)oxy)ethyl methanesulfonate

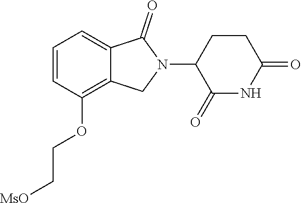 [SMILES: CS(=O)(=O)OCCOc1cccc2c1CN(C1CCC(=O)NC1=O)C2=O]

The titled compound (120 mg, 56%) was prepared in a manner similar to that in Example 207 step 4 from 3-(4-(2-hydroxyethoxy)-1-oxoisoindolin-2-yl)piperidine-2,6-dione and MsCl. [M+H]⁺=383.1.

### Step 6: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethoxy)-1-oxoisoindolin-2-yl)piperidine-2,6-dione

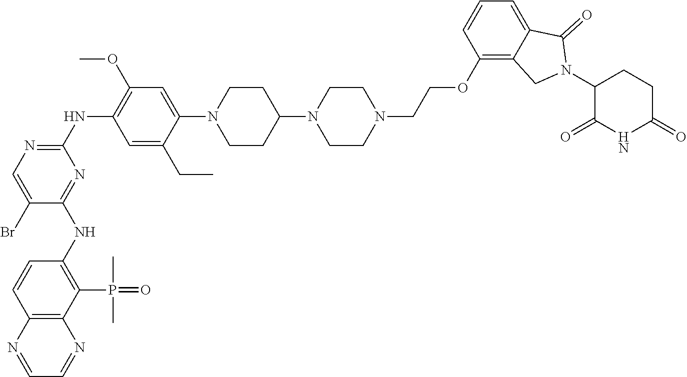 [SMILES: CCc1cc(Nc2ncc(Br)c(Nc3ccc4nccnc4c3P(C)(C)=O)n2)c(OC)cc1N1CCC(N2CCN(CCOc3cccc4c3CN(C3CCC(=O)NC3=O)C4=O)CC2)CC1]

The titled compound (15 mg, 26%) was prepared in a manner similar to that in Example 207 step 5 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 2-((2-(2,6-dioxopiperidin-3-yl)-1-oxoisoindolin-4-yl)oxy)ethyl methanesulfonate. ¹H NMR (400 MHz, DMSO) δ 12.64 (s, 1H), 10.97 (s, 1H), 8.86 (d, J=5.2 Hz, 3H), 8.26 (d, J=5.7 Hz, 2H), 7.90 (d, J=9.9 Hz, 1H), 7.48 (d, J=7.9 Hz, 1H), 7.42-7.23 (m, 3H), 6.80 (s, 1H), 5.11 (d, J=9.3 Hz, 1H), 4.38 (d, J=17.3 Hz, 1H), 4.25 (d, J=7.0 Hz, 3H), 3.77 (s, 3H), 2.95 (d, J=33.8 Hz, 5H), 2.70 (d, J=22.0 Hz, 5H), 2.57 (d, J=27.9 Hz, 8H), 2.31 (s, 2H), 2.02 (d, J=14.3 Hz, 6H), 1.84 (s, 2H), 1.57 (d, J=12.1 Hz, 2H), 0.92 (s, 3H). [M+H]⁺=982.9.

### Example 159: 5-(3-(((2R,6S)-4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)-2,6-dimethylpiperazin-1-yl)methyl)azetidin-1-yl)-2-(2,6-dioxopiperidin-3-yl)isoindoline-1,3-dione

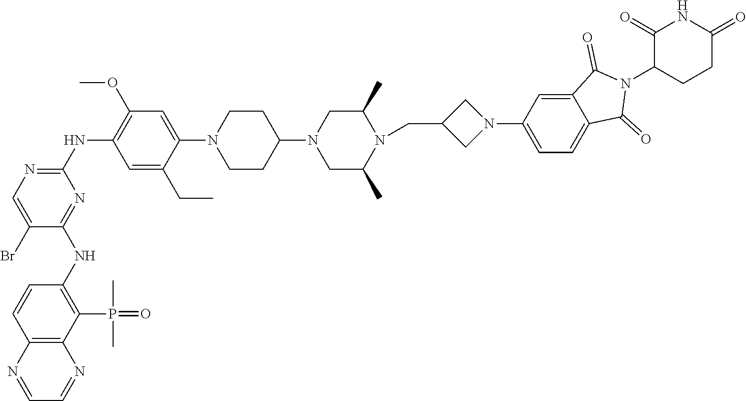 [SMILES: CCc1cc(Nc2ncc(Br)c(Nc3ccc4nccnc4c3P(C)(C)=O)n2)c(OC)cc1N1CCC(N2C[C@@H](C)N(CC3CN(c4ccc5c(c4)C(=O)N(C4CCC(=O)NC4=O)C5=O)C3)[C@@H](C)C2)CC1]

The titled compound was synthesized in the procedures similar to Example 59. ¹H NMR (500 MHz, DMSO) δ 12.65 (s, 1H), 11.09 (s, 1H), 8.85 (t, J=15.8 Hz, 3H), 8.29 (d, J=13.6 Hz, 2H), 7.89 (s, 1H), 7.64 (d, J=8.3 Hz, 1H), 7.37 (s, 1H), 6.73 (dd, J=72.3, 8.7 Hz, 3H), 5.06 (dd, J=12.7, 5.5 Hz, 1H), 4.13 (t, J=8.0 Hz, 2H), 3.77 (s, 3H), 3.72-3.62 (m, 2H), 3.01 (d, J=10.4 Hz, 3H), 2.87 (d, J=12.6 Hz, 4H), 2.77 (d, J=8.3 Hz, 2H), 2.73-2.61 (m, 5H), 2.27 (s, 2H), 2.09-2.00 (m, 10H), 1.83-1.82 (m, 2H), 1.57 (d, J=9.2 Hz, 2H), 1.09-1.00 (m, 6H), 0.95-0.89 (m, 3H). [M+H]⁺=1047.4

### Example 160: 3-(5-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)-1-oxoisoindolin-2-yl)piperidine-2,6-dione

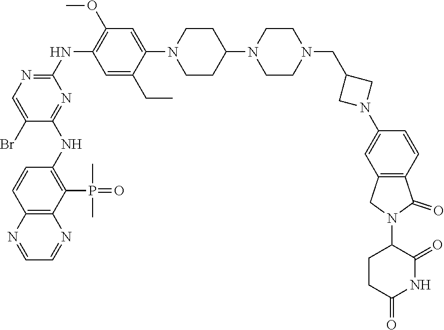 [SMILES: CCc1cc(Nc2ncc(Br)c(Nc3ccc4nccnc4c3P(C)(C)=O)n2)c(OC)cc1N1CCC(N2CCN(CC3CN(c4ccc5c(c4)CN(C4CCC(=O)NC4=O)C5=O)C3)CC2)CC1]

The titled compound was synthesized in the procedures similar to Example 237. ¹H NMR (400 MHz, DMSO) δ 12.65 (s, 1H), 10.94 (s, 1H), 8.87 (d, J=4.5 Hz, 3H), 8.27 (s, 2H), 7.90 (d, J=8.9 Hz, 1H), 7.48 (d, J=8.2 Hz, 1H), 7.38 (s, 1H), 6.81 (s, 1H), 6.50 (d, J=10.6 Hz, 2H), 5.03 (dd, J=12.9, 5.3 Hz, 1H), 4.28-4.38 (m, 1H), 4.19 (s, 1H), 4.02-4.05 (m, 2H), 3.77 (s, 3H), 3.56 (s, 2H), 2.91-3.10 (m, 6H), 2.71-2.80 (m, 3H), 2.58-2.61 (m, 6H), 2.42-2.45 (m, 6H), 2.02 (d, J=14.4 Hz, 6H), 1.97-1.90 (m, 1H), 1.85 (s, 2H), 1.58 (d, J=9.4 Hz, 2H), 0.93 (t, J=7.3 Hz, 3H); [M+H]⁺=1005.5.

### Example 165: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-5-chloro-2-fluorophenyl)piperidine-2,6-dione

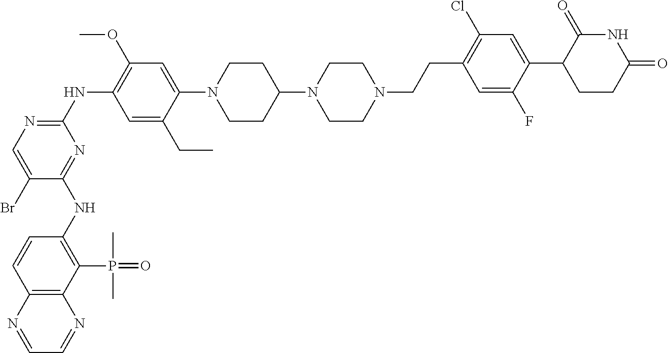 [SMILES: CCc1cc(Nc2ncc(Br)c(Nc3ccc4nccnc4c3P(C)(C)=O)n2)c(OC)cc1N1CCC(N2CCN(CCc3cc(F)c(C4CCC(=O)NC4=O)cc3Cl)CC2)CC1]

The titled compound was synthesized in the procedures similar to Example 200. ¹H NMR (400 MHz, DMSO) δ 12.65 (s, 1H), 10.92 (s, 1H), 8.87 (d, J=6.2 Hz, 2H), 8.85-8.78 (m, 1H), 8.28 (d, J=5.8 Hz, 2H), 7.91 (s, 1H), 7.42 (d, J=6.9 Hz, 1H), 7.38 (s, 1H), 7.30 (d, J=10.9 Hz, 1H), 6.81 (s, 1H), 4.05 (dd, J=12.7, 4.7 Hz, 1H), 3.77 (s, 3H), 3.30 (s, 3H), 3.02 (d, J=10.9 Hz, 2H), 2.85 (s, 2H), 2.72 (d, J=12.0 Hz, 3H), 2.64 (s, 1H), 2.58-2.52 (m, 4H), 2.49-2.45 (m, 5H), 2.36 (s, 1H), 2.24 (d, J=9.5 Hz, 1H), 2.02 (d, J=14.4 Hz, 7H), 1.88 (s, 2H), 1.61 (s, 2H), 0.93 (t, J=7.2 Hz, 3H); [M+H]⁺=961.4.

### Example 166: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2-chloro-5-fluorophenyl)piperidine-2,6-dione

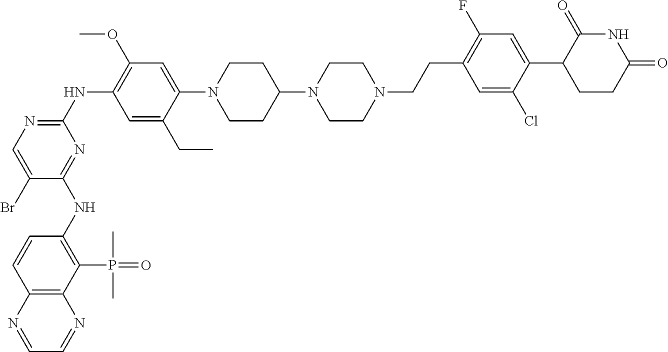 [SMILES: CCc1cc(Nc2ncc(Br)c(Nc3ccc4nccnc4c3P(C)(C)=O)n2)c(OC)cc1N1CCC(N2CCN(CCc3cc(Cl)c(C4CCC(=O)NC4=O)cc3F)CC2)CC1]

The titled compound was synthesized in the procedures similar to Example 200. ¹H NMR (400 MHz, DMSO) δ 12.64 (s, 1H), 10.91 (s, 1H), 8.86 (d, J=5.4 Hz, 3H), 8.27 (s, 2H), 7.90 (d, J=8.9 Hz, 1H), 7.48 (d, J=7.0 Hz, 1H), 7.38 (s, 1H), 7.22 (d, J=10.6 Hz, 1H), 6.81 (s, 1H), 4.18 (dd, J=12.5, 5.0 Hz, 1H), 3.77 (s, 3H), 3.01 (d, J=10.8 Hz, 2H), 2.76-2.71 (m, 6H), 2.56 (s, 7H), 2.48 (s, 5H), 2.35-2.30 (m, 2H), 2.02 (d, J=14.4 Hz, 6H), 1.99-1.94 (m, 1H), 1.86 (d, J=11.1 Hz, 2H), 1.58 (d, J=9.4 Hz, 2H), 0.93 (t, J=6.7 Hz, 3H); [M+H]⁺=961.6.

### Example 168: 3-(4-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)pyrrolidin-1-yl)phenyl)piperidine-2,6-dione

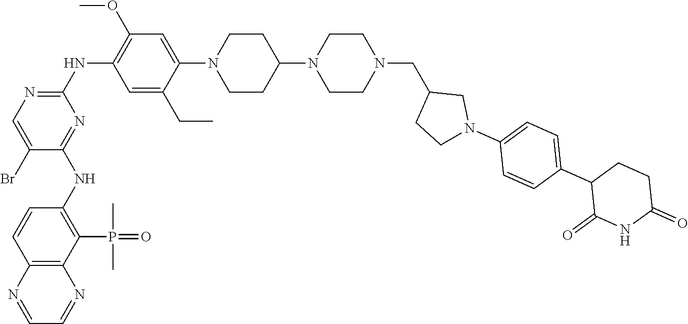 [SMILES: CCc1cc(Nc2ncc(Br)c(Nc3ccc4nccnc4c3P(C)(C)=O)n2)c(OC)cc1N1CCC(N2CCN(CC3CCN(c4ccc(C5CCC(=O)NC5=O)cc4)C3)CC2)CC1]

The titled compound was synthesized in the procedures similar to Example 207. ¹H NMR (400 MHz, DMSO) δ 12.65 (s, 1H), 10.74 (s, 1H), 8.87 (d, J=5.6 Hz, 3H), 8.27 (s, 2H), 7.92 (s, 1H), 7.39 (s, 1H), 6.99 (d, J=8.5 Hz, 2H), 6.81 (s, 1H), 6.49 (s, 2H), 3.77 (s, 3H), 3.67 (s, 1H), 3.34 (s, 3H), 3.24-3.17 (m, 1H), 3.02 (s, 6H), 2.72 (s, 3H), 2.53 (s, 6H), 2.33 (s, 6H), 2.02 (d, J=14.5 Hz, 9H), 1.93-1.82 (m, 1H), 1.79-1.52 (m, 3H), 0.93 (t, J=7.2 Hz, 3H); [M+H]⁺=964.4.

### Example 178: 3-(4-(3-(4-(4-(4-((5-bromo-4-((5-(dimethylphosphoryl)quinoxalin-6-yl)amino)pyrimidin-2-yl)amino)-2-ethyl-5-methoxyphenyl)piperazin-1-yl)piperidin-1-yl)cyclopentyl)phenyl)piperidine-2,6-dione

### Step 1: 3-(4-(2,6-bis(benzyloxy)pyridin-3-yl)phenyl)cyclopentan-1-one

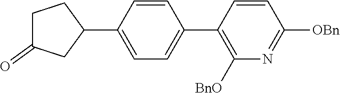 [SMILES: O=C1CCC(c2ccc(-c3ccc(OCc4ccccc4)nc3OCc3ccccc3)cc2)C1]

A mixture of 3-(4-bromophenyl)cyclopentan-1-one (2.39 g, 10.0 mmol), 2,6-bis(benzyloxy)-3-(4,4,5,5-tetramethyl-1,3,2-dioxaborolan-2-yl)pyridine (4.17 g, 10.0 mmol), Cs₂CO₃ (6.50 g, 20.0 mmol) and Pd(DPPF)Cl₂ (0.73 g, 1.0 mmol) in dioxane (80 mL) and water (20 mL) was stirred in a round bottom flask at 95° C. for 18 hours. The mixture was evaporated in vacuum to afford the crude product, which was further purified with silica gel column chromatography (PE: EA=100: 1˜ 50: 50 gradient elution) to give the title product (4.02 g, 81%). [M+H]⁺=449.8.

### Step 2: 3-(4-(3-oxocyclopentyl)phenyl)piperidine-2,6-dione

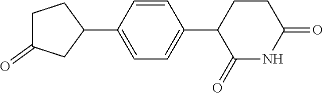 [SMILES: O=C1CCC(c2ccc(C3CCC(=O)NC3=O)cc2)C1]

To a solution of 3-(4-(2,6-bis(benzyloxy)pyridin-3-yl)phenyl)cyclopentan-1-one (4.02 g, 8.95 mmol) in DCM (100 mL) and MeOH (100 mL) was added Pd/C (400 mg) and stirred at 25° C. for 18 hours under one balloon H₂. The mixture was filtered and evaporated in vacuum to afford the crude product (2.20 g, 90%). [M+H]⁺=271.8.

### Step 3: 3-(4-(3-(4-(4-(4-((5-bromo-4-((5-(dimethylphosphoryl)quinoxalin-6-yl)amino)pyrimidin-2-yl)amino)-2-ethyl-5-methoxyphenyl)piperazin-1-yl)piperidin-1-yl)cyclopentyl)phenyl)piperidine-2,6-dione

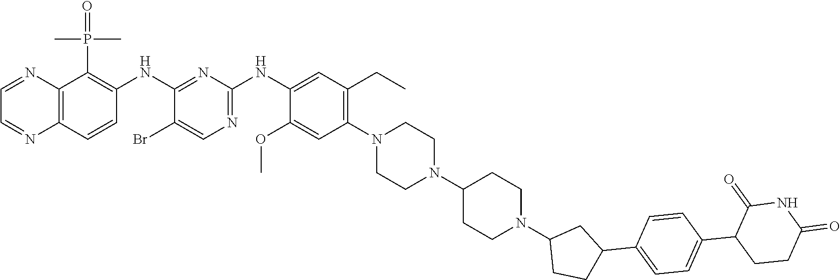 [SMILES: CCc1cc(Nc2ncc(Br)c(Nc3ccc4nccnc4c3P(C)(C)=O)n2)c(OC)cc1N1CCN(C2CCN(C3CCC(c4ccc(C5CCC(=O)NC5=O)cc4)C3)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (36 mg, 0.05 mmol) in DCM (6 mL) and EtOH (1 mL) was added 3-(4-(3-oxocyclopentyl)phenyl)piperidine-2,6-dione (27 mg, 0.10 mmol) then stirred in a round bottom flask at room temperature for 1 hour. The mixture was added NaBH(OAc)₃ (106 mg, 0.50 mmol) and stirred in a round bottom flask at room temperature 18 hours. Then the mixture was evaporated in vacuum to afford the crude product, which was purified with Pre-TLC (DCM: MeOH=7:1) to give the product (5.0 mg, 10%). ¹H NMR (400 MHz, DMSO) δ 12.65 (s, 1H), 10.83 (s, 1H), 9.04-8.69 (m, 2H), 8.39-8.20 (m, 2H), 8.01-7.86 (m, 1H), 7.45-7.35 (m, 1H), 7.30-7.21 (m, 2H), 7.16 (s, 2H), 6.81 (s, 1H), 3.93-3.70 (m, 4H), 3.60-3.43 (m, 1H), 3.31-3.26 (m, 3H), 3.05 (s, 8H), 2.82-2.59 (m, 5H), 2.47-2.36 (m, 3H), 2.26-2.11 (m, 3H), 2.07-1.97 (m, 8H), 1.94-1.76 (m, 3H), 1.71-1.47 (m, 3H), 1.34-1.17 (m, 2H), 0.98-0.83 (m, 3H). [M+H]⁺=949.8.

### Example 199: 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)phenyl)piperidine-2,6-dione

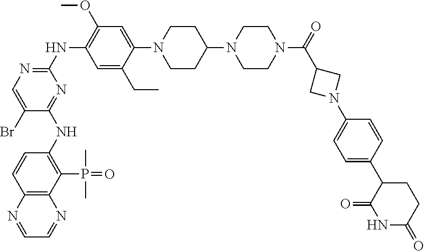 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc(C5CCC(=O)NC5=O)cc4)C3)CC2)CC1]

The titled compound was prepared in a manner similar to that in Example 208. ¹H NMR (400 MHz, DMSO) δ 12.65 (s, 1H), 10.76 (s, 1H), 8.85 (dt, J=18.6, 9.3 Hz, 3H), 8.27 (s, 2H), 7.90 (d, J=9.8 Hz, 1H), 7.38 (s, 1H), 7.02 (d, J=8.5 Hz, 2H), 6.81 (s, 1H), 6.42 (d, J=8.4 Hz, 2H), 4.01 (t, J=5.6 Hz, 2H), 3.89-3.80 (m, 3H), 3.77 (s, 3H), 3.73-3.67 (m, 1H), 3.49 (s, 2H), 3.02 (d, J=11.1 Hz, 2H), 2.77-2.54 (m, 7H), 2.48-2.36 (m, 3H), 2.17-1.94 (m, 12H), 1.85-1.90 (m, 2H), 1.67-1.53 (m, 2H), 0.93-0.98 (m, 3H); [M+H]⁺=964.5.

### Example 205: 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)propyl)-2,6-difluorophenyl)piperidine-2,6-dione

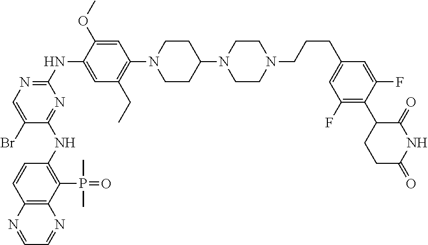 [SMILES: CCc1cc(Nc2ncc(Br)c(Nc3ccc4nccnc4c3P(C)(C)=O)n2)c(OC)cc1N1CCC(N2CCN(CCCc3cc(F)c(C4CCC(=O)NC4=O)c(F)c3)CC2)CC1]

The titled compound was prepared in a manner similar to that in Example 200. ¹H NMR (500 MHz, DMSO) δ 12.64 (s, 1H), 10.94 (s, 1H), 8.89-8.75 (m, 3H), 8.27 (s, 2H), 7.90 (d, J=9.0 Hz, 1H), 7.38 (s, 1H), 7.00 (d, J=10.0 Hz, 2H), 6.81 (s, 1H), 4.19 (dd, J=12.5, 5.0 Hz, 1H), 3.77 (s, 3H), 3.05-2.97 (m, J=11.5 Hz, 2H), 2.85-2.77 (m, 1H), 2.74-2.67 (m, 2H), 2.63-2.59 (m, 2H), 2.59-2.52 (m, 4H), 2.48-2.34 (m, 7H), 2.32-2.25 (m, 3H), 2.16-2.08 (m, 1H), 2.04-1.97 (m, 7H), 1.89-1.83 (m, 2H), 1.78-1.70 (m, 2H), 1.62-1.52 (m, 2H), 0.96-0.87 (m, 3H). [M+H]⁺=959.50.

### Example 206: 3-(4-(2-(4-(1-(4-((5-bromo-4-((4-(dimethylphosphoryl)benzo[d]thiazol-5-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: 4-iodobenzo[d]thiazol-5-amine

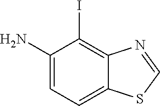 [SMILES: Nc1ccc2scnc2c1I]

To a mixture of benzo[d]thiazol-5-amine (15 g, 100 mmol), NaHCO₃ (16.8 g, 200 mmol) in CH₂Cl₂ 250 mL was added ICl (17.8 g, 110 mmol) portionwise at 5° C. for 30 minutes. The mixture was stirred at 20° C. for 2 hours. The mixture was quenched by saturated brine 100 mL and saturated Na₂S₂O₃ aqueous solution 50 mL, and then extracted with DCM (3×100 mL). The combined organic layer was dried over Na₂SO₄, filtered and concentrated. The residue was purified with silica gel column (MeOH in DCM, 0% to 2%) to afford the product (22.8 g, 82.6%). [M+H]⁺=277.2.

### Step 2: (5-aminobenzo[d]thiazol-4-yl)dimethylphosphine oxide

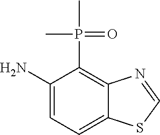

A mixture of 4-iodobenzo[d]thiazol-5-amine (22.8 g, 82.5 mmol), dimethylphosphine oxide (9.67 g, 123.8 mmol), Pd(OAc)₂ (1.85 g, 8.25 mmol), Xantphos (9.55 g, 16.52 mmol), K₃PO₄ (43.8 g, 206 mmol) in DMF (150 mL) was stirred at 120° C. overnight. The solvent was removed by rotary concentration. The residue was diluted with water 100 mL and extracted with EtOAc (3×200 mL). The combined organic layer was dried over Na₂SO₄, filtered and concentrated. The residue was purified with silica gel column (MeOH in DCM, 0% to 6%) to afford the product (15.6 g, 83.5%). [M+H]⁺=227.2.

### Step 3: (5-((5-bromo-2-chloropyrimidin-4-yl)amino)benzo[d]thiazol-4-yl)dimethylphosphine oxide

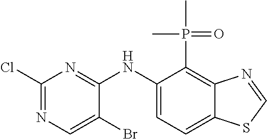 [SMILES: CP(C)(=O)c1c(Nc2nc(Cl)ncc2Br)ccc2scnc12]

A mixture of (5-aminobenzo[d]thiazol-4-yl)dimethylphosphine oxide (1.13 g, 5 mmol), 5-bromo-2,4-dichloropyrimidine (1.37 g, 6 mmol), Et₃N (1.52 g, 15 mmol) in n-BuOH 100 mL was stirred at 80° C. for 24 hours. The solvent was removed by rotary concentration. The residue was purified with silica gel column (MeOH in DCM, 0% to 5%) to afford product (1.8 g, 86.3%). [M+H]⁺=416.9.

### Step 4: tert-butyl 4-(1-(4-((5-bromo-4-((4-(dimethylphosphoryl)benzo[d]thiazol-5-yl)amino)pyrimidin-2-yl)amino)-2-ethyl-5-methoxyphenyl)piperidin-4-yl)piperazine-1-carboxylate

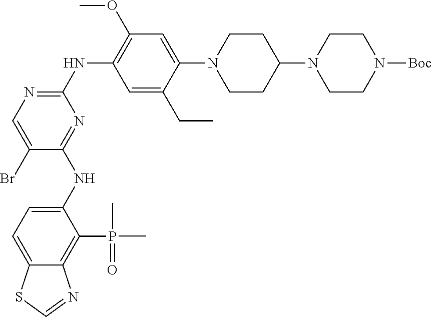 [SMILES: CCc1cc(Nc2ncc(Br)c(Nc3ccc4scnc4c3P(C)(C)=O)n2)c(OC)cc1N1CCC(N2CCN(C(=O)OC(C)(C)C)CC2)CC1]

A mixture of (5-((5-bromo-2-chloropyrimidin-4-yl)amino)benzo[d]thiazol-4-yl)dimethylphosphine oxide (0.48 g, 1.15 mmol), tert-butyl 4-(1-(4-amino-2-ethyl-5-methoxyphenyl)piperidin-4-yl)piperazine-1-carboxylate (0.53 g, 1.26 mmol) and TsOH (0.39 g, 2.3 mmol) in dioxane 100 mL was stirred at 95° C. for 60 hours. The mixture was concentrated and diluted with 1N aqueous NaOH (50 mL), extracted with DCM (3×100 mL). The combined organic layer was dried over Na₂SO₄, filtered and concentrated. The residue was purified with silica gel column (MeOH in DCM, 0% to 5%) to afford product (238 mg, 25.9%). [M+H]⁺=799.4.

### Step 5: (5-((5-bromo-2-((5-ethyl-2-methoxy-4-(4-(piperazin-1-yl)piperidin-1-yl)phenyl)amino)pyrimidin-4-yl)amino)benzo[d]thiazol-4-yl)dimethylphosphine oxide

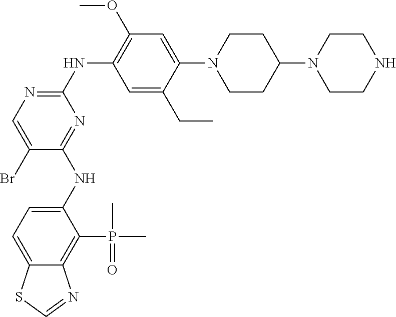 [SMILES: CCc1cc(Nc2ncc(Br)c(Nc3ccc4scnc4c3P(C)(C)=O)n2)c(OC)cc1N1CCC(N2CCNCC2)CC1]

To a solution of tert-butyl 4-(1-(4-((5-bromo-4-((4-(dimethylphosphoryl)benzo[d]thiazol-5-yl)amino)pyrimidin-2-yl)amino)-2-ethyl-5-methoxyphenyl)piperidin-4-yl)piperazine-1-carboxylate in EtOH (30 mL) was added a solution of HCl (4N, 10 mL) in dioxane. The resulting solution was stirred at rt for 2 hours. The mixture was concentrated to dryness and 1N NaOH aqueous (10 mL) was added. The mixture was extracted with DCM (3×50 mL). The combined organic layer was dried over Na₂SO₄, filtered and concentrated to afford product (180 mg, 86.4%). [M+H]⁺=699.4.

### Step 6: 3-(4-(2-(4-(1-(4-((5-bromo-4-((4-(dimethylphosphoryl)benzo[d]thiazol-5-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

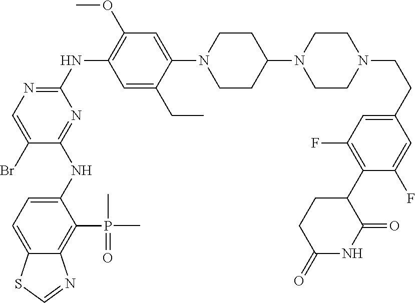 [SMILES: CCc1cc(Nc2ncc(Br)c(Nc3ccc4scnc4c3P(C)(C)=O)n2)c(OC)cc1N1CCC(N2CCN(CCc3cc(F)c(C4CCC(=O)NC4=O)c(F)c3)CC2)CC1]

A solution of (5-((5-bromo-2-((5-ethyl-2-methoxy-4-(4-(piperazin-1-yl)piperidin-1-yl)phenyl)amino)pyrimidin-4-yl)amino)benzo[d]thiazol-4-yl)dimethylphosphine oxide (100 mg, 0.143 mmol), 2-(4-(2,6-dioxopiperidin-3-yl)-3,5-difluorophenyl)acetaldehyde (57 mg, 0.214 mmol) and HOAc (1.7 mg, 0.0286 mmol) in DCM/EtOH (30/30 mL) was stirred at 20° C. for 3 hours. Then NaBH(OAc)₃ (60.6 mg, 0.286 mmol) was added to the mixture at 20° C. The solution was stirred at 20° C. for 9 hrs. The reaction was quenched by aqueous Na₂CO₃ (10 mL), extracted with DCM (3×100 mL). The combined organic phase dried over Na₂SO₄, filtered and concentrated in vacuum. The residue was purified by Prep. HPLC (ACN in water with 0.1% of FA, 0% to 90%). 3-(4-(2-(4-(1-(4-((5-bromo-4-((4-(dimethylphosphoryl)benzo[d]thiazol-5-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione (86.9 mg, 64.0%) was obtained. ¹H NMR (500 MHz, DMSO) δ 11.90 (s, 1H), 10.95 (s, 1H), 9.50 (s, 1H), 8.49 (s, 1H), 8.18 (s, 1H), 8.11 (d, 2H), 7.45 (s, 1H), 7.03 (d, 2H), 6.77 (s, 1H), 4.20 (dd, 1H), 3.77 (s, 3H), 3.01-2.99 (m, 2H), 2.83-2.70 (m, 3H), 2.69-2.64 (m, 2H), 2.60-2.54 (m, 7H), 2.45-2.40 (m, 7H), 2.34-2.30 (m, 1H), 2.14-2.11 (m, 1H), 2.04 (s, 3H), 1.98 (s, 3H), 1.87-1.85 (m, 2H), 1.56 (dd, 2H), 0.92 (t, 3H); [M+H]⁺=950.4.

### Example 207: 5-(3-((4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)methyl)azetidin-1-yl)-2-(2,6-dioxopiperidin-3-yl)benzonitrile

### Step 1: 2-(2,6-bis(benzyloxy)pyridin-3-yl)-5-bromobenzonitrile

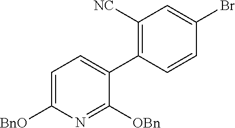 [SMILES: N#Cc1cc(Br)ccc1-c1ccc(OCc2ccccc2)nc1OCc1ccccc1]

To a solution of 2,6-bis(benzyloxy)-3-(4,4,5,5-tetramethyl-1,3,2-dioxaborolan-2-yl)pyridine (8.34 g, 20 mmol), 5-bromo-2-iodobenzonitrile (6.75 g, 22 mmol) and K₂CO₃ (5.52 g, 40 mmol) in 50 mL dioxane and 5 mL water, Pd(dppf)Cl₂ (731 mg, 1 mmol) was added under N₂ protected. The mixture was stirred at 90° C. for 16 hours under N₂ atmosphere. The mixture was concentrated in vacuum. The residue was washed with water (100 mL) and extracted by EtOAc (100 mL×3). The combined organic phase was washed with brine (100 mL), dried over Na₂SO₄, filtered and concentrated in vacuum. The residue was purified by silica solumn chromatography (PE:EA=20:1) to afford product (8.5 g, 90.4% yield). [M+H]⁺=471.6.

### Step 2: 2-(2,6-bis(benzyloxy)pyridin-3-yl)-5-(3-(hydroxymethyl)azetidin-1-yl)benzonitrile

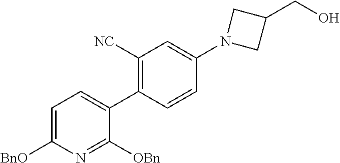 [SMILES: N#Cc1cc(N2CC(CO)C2)ccc1-c1ccc(OCc2ccccc2)nc1OCc1ccccc1]

To a solution of 2-(2,6-bis(benzyloxy)pyridin-3-yl)-5-bromobenzonitrile (3.1 g, 6.85 mmol), azetidin-3-ylmethanol hydrochloride (984 mg, 8 mmol) and K₃PO₄ (3.4 g, 16 mmol) in 20 mL DMSO, CuI (190 mg, 1 mmol) and L-Proline (230 mg, 2 mmol) was added under N₂ protected. The mixture was stirred at 100° C. for 16 hours under N₂ atmosphere. The mixture was diluted with EtOAc (150 mL) and washed with brine (100 mL×2). The organic phase was dried over Na₂SO₄, filtered and concentrated in vacuum. The residue was purified by silica column chromatography (PE:EA=5:1) to afford product (2.1 g, 64.3% yield). [M+H]⁺=477.6.

### Step 3: 2-(2,6-dioxopiperidin-3-yl)-5-(3-(hydroxymethyl)azetidin-1-yl)benzonitrile

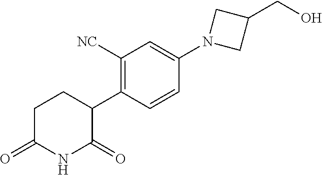 [SMILES: N#Cc1cc(N2CC(CO)C2)ccc1C1CCC(=O)NC1=O]

To a solution of 2-(2,6-bis(benzyloxy)pyridin-3-yl)-5-(3-(hydroxymethyl)azetidin-1-yl)benzonitrile (500 mg, 1.05 mmol) in EtOAc (50 mL), 500 mg Pd/C was added. The mixture was stirred at 50° C. for 2 days under H₂ atmosphere. The mixture was filtered through celite. Pd/C was washed with 50 mL solvent (DCM:MeOH=1:10) twice. The combined organic phase was concentrated in vacuum to afford product (100 mg, 32.2% yield). [M+H]⁺=299.6.

### Step 4: (1-(3-cyano-4-(2,6-dioxopiperidin-3-yl)phenyl)azetidin-3-yl)methyl methanesulfonate

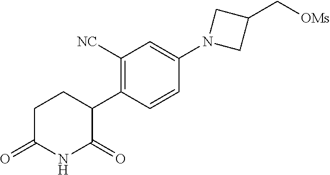 [SMILES: CS(=O)(=O)OCC1CN(c2ccc(C3CCC(=O)NC3=O)c(C#N)c2)C1]

To a solution of 2-(2,6-dioxopiperidin-3-yl)-5-(3-(hydroxymethyl)azetidin-1-yl)benzonitrile (100 mg, 0.33 mmol) and Et₃N (100 mg, 1 mmol) in 10 mL DCM, MsCl (40 mg, 0.35 mmol) was slowly added at 0° C. The mixture was stirred at 25° C. for 2 hours. After LCMS shown the reaction was completed. The mixture was quenched with 10 mL water. The organic phase was separated and concentrated in vacuum. The residue was purified by prep-TLC (DCM:MeOH=20:1) to afford product (44 mg, 35.3% yield). [M+H]⁺=377.6.

### Step 5: 5-(3-((4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)methyl)azetidin-1-yl)-2-(2 ,6-dioxopiperidin-3-yl)benzonitrile

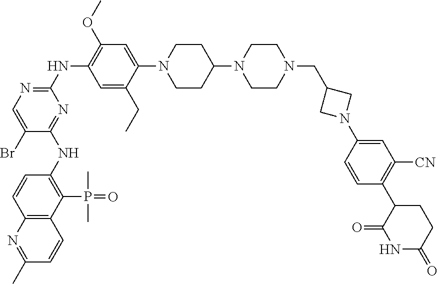 [SMILES: CCc1cc(Nc2ncc(Br)c(Nc3ccc4nc(C)ccc4c3P(C)(C)=O)n2)c(OC)cc1N1CCC(N2CCN(CC3CN(c4ccc(C5CCC(=O)NC5=O)c(C#N)c4)C3)CC2)CC1]

To a solution of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (50 mg, 0.07 mmol), (1-(3-cyano-4-(2,6-dioxopiperidin-3-yl)phenyl)azetidin-3-yl)methyl methanesulfonate (26 mg, 0.07 mmol), KI (33 mg, 0.2 mmol) and DIEA (39 mg, 0.3 mmol) in 5 mL ACN. The mixture was stirred at 80° C. for 16 hours. After LCMS shown the reaction was completed. The mixture was concentrated in vacuum. The residue was dissolved in DCM and filtered. The organic phase was concentrated in vacuum and purified by prep-HPLC with C-18 column chromatography (0.1% FA in water: acetonitrile=90: 10˜ 60: 40 gradient elution) to afford product (7.06 mg, 10.2% yield). ¹H NMR (500 MHz, DMSO) δ 11.76 (s, 1H), 10.90 (s, 1H), 8.56 (d, J=8.9 Hz, 1H), 8.30 (d, J=7.6 Hz, 1H), 8.21 (s, 1H), 7.98 (s, 1H), 7.87 (d, J=9.3 Hz, 1H), 7.42 (d, J=8.9 Hz, 1H), 7.37 (s, 1H), 7.22 (d, J=8.6 Hz, 1H), 6.79 (d, J=2.4 Hz, 1H), 6.74 (s, 1H), 6.69 (dd, J=8.6, 2.4 Hz, 1H), 4.01-3.93 (m, 3H), 3.75 (s, 3H), 3.54-3.46 (m, 2H), 2.94 (d, J=10.9 Hz, 3H), 2.85-2.74 (m, 1H), 2.69-2.61 (m, 5H), 2.59-2.51 (m, 7H), 2.38 (ddd, J=9.0, 5.9, 5.4 Hz, 2H), 2.33-2.22 (m, 4H), 2.04-1.93 (m, 7H), 1.83 (d, J=10.1 Hz, 2H), 1.60-1.48 (m, 2H), 0.82-0.72 (m, 3H). [M+H]⁺=988.6.

### Example 209: 3-(4-(((1r,3r)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)cyclobutyl)amino)-2,6-difluorophenyl)piperidine-2,6-dione

### Step 1: methyl (1r,3r)-3-((4-(2,6-bis(benzyloxy)pyridin-3-yl)-3,5-difluorophenyl)amino)cyclobutane-1-carboxylate

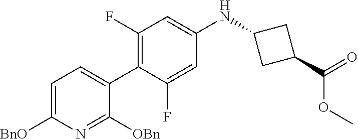 [SMILES: COC(=O)[C@H]1C[C@H](Nc2cc(F)c(-c3ccc(OCc4ccccc4)nc3OCc3ccccc3)c(F)c2)C1]

To a solution of 2,6-bis(benzyloxy)-3-(4-bromo-2,6-difluorophenyl)pyridine (1 g, 2.07 mmol), methyl (1r,3r)-3-aminocyclobutane-1-carboxylate hydrochloride (495 mg, 3 mmol) and Cs₂CO₃ (1.95 g, 6 mmol) in 10 mL DMSO, Pd₂(dba)₃ (183 mg, 0.2 mmol) and Xantphos (231 mg, 0.4 mmol) was added under N₂ protected. The mixture was stirred at 80° C. for 16 hours. The mixture was diluted with EtOAc (100 mL) and washed with brine (100 mL×2). The organic phase was dried over Na₂SO₄, filtered and concentrated in vacuum. The residue was purified by silica column chromatography (PE:EA=20:1) to afford product (351 mg, 32% yield). [M+H]⁺=530.8.

### Step 2: (1r,3r)-3-((4-(2,6-bis(benzyloxy)pyridin-3-yl)-3,5-difluorophenyl)amino)cyclobutane-1-carboxylic acid

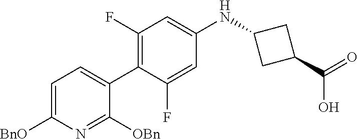 [SMILES: O=C(O)[C@H]1C[C@H](Nc2cc(F)c(-c3ccc(OCc4ccccc4)nc3OCc3ccccc3)c(F)c2)C1]

To a solution of methyl (1r,3r)-3-((4-(2,6-bis(benzyloxy)pyridin-3-yl)-3,5-difluorophenyl)amino)cyclobutane-1-carboxylate (351 mg, 0.66 mmol) in 10 mL THF, LiOH H₂O (84 mg, 2 mmol) in 2 mL water was added. The mixture was stirred at 25° C. for 2 hours. After LCMS showed the reaction was completed. The mixture was concentrated in vacuum. The residue was adjust pH<5 with 1 N HCl and extracted with 50 mL EtOAc. The organic phase was dried over Na₂SO₄, filtered and concentrated in vacuum to afford product (320 mg, 94% yield). [M+H]⁺=516.8.

### Step 3: (1r,3r)-3-((4-(2,6-dioxopiperidin-3-yl)-3,5-difluorophenyl)amino)cyclobutane-1-carboxylic acid

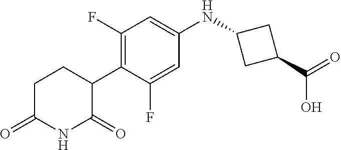 [SMILES: O=C1CCC(c2c(F)cc(N[C@H]3C[C@H](C(=O)O)C3)cc2F)C(=O)N1]

To a solution of (1r,3r)-3-((4-(2,6-bis(benzyloxy)pyridin-3-yl)-3,5-difluorophenyl)amino)cyclobutane-1-carboxylic acid (320 mg, 0.62 mmol) in 5 mL DCM and 30 mL MeOH, 160 mg Pd/C was added. The mixture was stirred at 30° C. for 16 hours under H₂ atmosphere. The mixture was filtered through celite. Pd/C was washed with 50 mL solvent (DCM:MeOH=1:10) twice and filtered. The organic phase was concentrated in vacuum to afford (1r,3r)-3-((4-(2,6-dioxopiperidin-3-yl)-3,5-difluorophenyl)amino)cyclobutane-1-carboxylic acid (153 mg, 73% yield). [M+H]⁺=338.8.

### Step 4: 3-(4-(((1r,3r)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)cyclobutyl)amino)-2,6-difluorophenyl)piperidine-2,6-dione

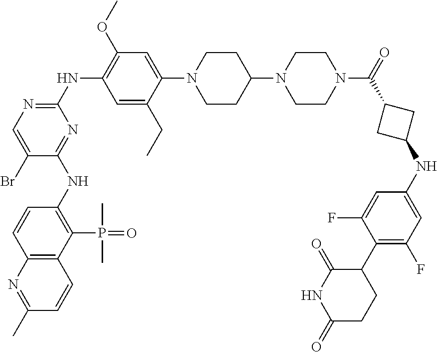 [SMILES: CCc1cc(Nc2ncc(Br)c(Nc3ccc4nc(C)ccc4c3P(C)(C)=O)n2)c(OC)cc1N1CCC(N2CCN(C(=O)[C@H]3C[C@H](Nc4cc(F)c(C5CCC(=O)NC5=O)c(F)c4)C3)CC2)CC1]

To a solution of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (50 mg, 0.07 mmol), (1r,3r)-3-((4-(2,6-dioxopiperidin-3-yl)-3,5-difluorophenyl)amino)cyclobutane-1-carboxylic acid (24 mg, 0.07 mmol) and DIEA (26 mg, 0.2 mmol) in 10 mL DCM, 50% w.t. T₃P in EtOAc solution (64 mg, 0.1 mmol) was added. The mixture was stirred at 25° C. for 16 hours. The mixture was quenched with water (10 mL). The organic phase was concentrated in vacuum and purified by prep-HPLC with C-18 column chromatography (0.1% FA in water: acetonitrile=90: 10˜ 50: 50 gradient elution) to afford product (8.2 mg, 11.4% yield). ¹H NMR (500 MHz, DMSO) δ 11.76 (s, 1H), 10.83 (s, 1H), 8.56 (d, J=8.7 Hz, 1H), 8.30 (d, J=7.0 Hz, 1H), 8.21 (s, 1H), 7.97 (s, 1H), 7.86 (d, J=9.1 Hz, 1H), 7.46-7.30 (m, 2H), 6.73 (s, 1H), 6.58 (dd, J=39.3, 6.3 Hz, 1H), 6.13 (dd, J=25.1, 11.7 Hz, 2H), 3.98 (dd, J=12.5, 4.4 Hz, 1H), 3.81-3.70 (m, 4H), 3.52-3.43 (m, 2H), 3.40 (s, 2H), 3.04 (dt, J=17.7, 8.9 Hz, 1H), 2.94 (d, J=10.2 Hz, 2H), 2.82-2.72 (m, 1H), 2.66 (d, J=16.8 Hz, 5H), 2.60-2.52 (m, 3H), 2.48-2.46 (m, 4H), 2.40-2.26 (m, 3H), 2.12-1.89 (m, 10H), 1.81 (d, J=11.1 Hz, 2H), 1.56 (dd, J=21.3, 11.6 Hz, 2H), 0.90-0.62 (m, 3H). [M+H]⁺=1027.6.

### Example 210: 3-(5-(4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)piperidin-1-yl)-3-methyl-2-oxo-2,3-dihydro-1H-benzo[d]imidazol-1-yl)piperidine-2,6-dione

### Step 1: 6-bromo-1-methyl-3H-1,3-benzodiazol-2-one

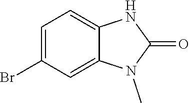

Into a 1000-mL round-bottom flask, were placed 5-bromo-N1-methylbenzene-1,2-diamine (10.0 g, 50.0 mmol), CH₃CN (200 mL), 1,1′-Carbonyldiimidazole (9.4 g, 58.0 mmol). The resulting solution was stirred for 5 h at 70° C. The filtrate was concentrated after filtration. This resulted in 10.0 g (88.6%) of 6-bromo-1-methyl-3H-1,3-benzodiazol-2-one. ¹HNMR (500 MHz, DMSO-d6) δ ppm 10.98 (s, 1H), 7.31 (t, J=1.5 Hz, 1H), 7.11 (ddd, J=8.2 Hz, 1H), 6.91 (d, J=8.2 Hz, 1H), 3.27 (s, 3H). [M+H]⁺=227.2.

### Step 2: 3-(5-bromo-3-methyl-2-oxo-2,3-dihydro-1H-benzo[d]imidazol-1-yl)-1-(4-methoxybenzyl)piperidine-2,6-dione

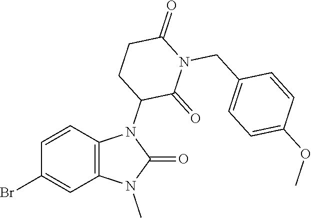 [SMILES: COc1ccc(CN2C(=O)CCC(n3c(=O)n(C)c4cc(Br)ccc43)C2=O)cc1]

Into a 500-mL round-bottom flask purged and maintained with an inert atmosphere of nitrogen, were placed 6-bromo-1-methyl-3H-1,3-benzodiazol-2-one (10.0 g, 44.0 mmol), THF (100 mL), t-BuOK (4.9 g, 44.0 mmol), the resulting solution was stirred for 0.5 h at 0° C. and then was added 3-bromo-1-((4-methoxyphenyl)methyl)piperidine-2,6-dione (13.7 g, 44.0 mmol). The resulting solution was allowed to react, with stirring, for an additional 3 days at rt. The resulting solution was diluted with (200 mL) of EtOAc. The resulting mixture was washed with (3×200 m1) of H₂O. The organic phase was dried over anhydrous sodium sulfate and concentrated. The residue was purified by combi-flash (EtOAc/PE=0-50%) to give the product (2.2 g, 11% yield). ¹H NMR (500 MHz, DMSO-d6) δ ppm 7.48 (d, J=1.9 Hz, 1H), 7.27-7.14 (m, 3H), 7.02 (d, J=8.4 Hz, 1H), 6.97-6.81 (m, 2H), 4.89-4.71 (m, 2H), 3.74 (s, 3H), 3.36 (s, 3H), 3.15-2.97 (m, 1H), 2.89-2.78 (m, 1H), 2.72 (td, J=13.1 Hz, 1H), 2.13-1.97 (m, 1H). [M+H]⁺=458.1.

### Step 3: tert-butyl 1-(1-(1-(4-methoxybenzyl)-2,6-dioxopiperidin-3-yl)-3-methyl-2-oxo-2,3-dihydro-1H-benzo[d]imidazol-5-yl)piperidine-4-carboxylate

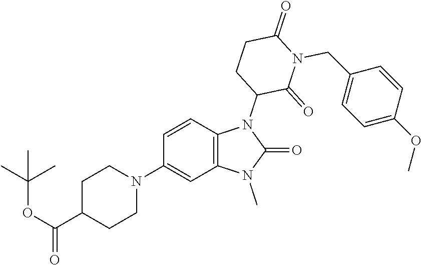 [SMILES: COc1ccc(CN2C(=O)CCC(n3c(=O)n(C)c4cc(N5CCC(C(=O)OC(C)(C)C)CC5)ccc43)C2=O)cc1]

Into a 50-mL vial purged and maintained with an inert atmosphere of nitrogen, were placed 3-(5-bromo-3-methyl-2-oxo-2,3-dihydro-1H-benzo[d]imidazol-1-yl)-1-(4-methoxybenzyl)piperidine-2,6-dione (800.0 mg, 1.7 mmol), dioxane (20 mL), tert-butyl piperidine-4-carboxylate (485.1 mg, 2.6 mmol), Cs₂CO₃ (1.1 g, 3.5 mmol), Ruphos-G3 (225.2 mg, 0.3 mmol). The resulting solution was stirred for 15 h at 100° C. The resulting solution was diluted with EtOAc (50 mL). The resulting mixture was washed with water (3×50 mL). The organic phase was dried over anhydrous sodium sulfate and concentrated. The residue was purified by combi-flash (EtOAc/PE=0-25%) to give the product (400 mg, 41% yield). [M+H]⁺=563.2.

### Step 4: 1-(1-(2,6-dioxopiperidin-3-yl)-3-methyl-2-oxo-2,3-dihydro-1H-benzo[d]imidazol-5-yl)piperidine-4-carboxylic acid

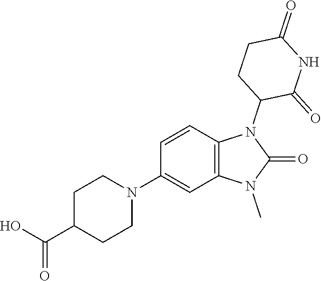 [SMILES: Cn1c(=O)n(C2CCC(=O)NC2=O)c2ccc(N3CCC(C(=O)O)CC3)cc21]

Into a 10-mL vial purged and maintained with an inert atmosphere of nitrogen, were placed tert-butyl 1-(1-(1-(4-methoxybenzyl)-2,6-dioxopiperidin-3-yl)-3-methyl-2-oxo-2,3-dihydro-1H-benzo[d]imidazol-5-yl)piperidine-4-carboxylate (300.0 mg, 0.53 mmol), Toluene (2 mL), CH₃SO₃H (1 mL). The resulting solution was stirred for 2 h at 100° C. The resulting solution was diluted with EtOAc (10 mL). The solids were collected by filtration. The solids were washed with EtOAc (3×20 mL). The solids were dried in vacuum. The product 1-(1-(2,6-dioxopiperidin-3-yl)-3-methyl-2-oxo-2,3-dihydro-1H-benzo[d]imidazol-5-yl)piperidine-4-carboxylic acid (190 mg, 92.22%) was obtained. [M+H]⁺=387.2.

### Step 5: 3-(5-(4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)piperidin-1-yl)-3-methyl-2-oxo-2,3-dihydro-1H-benzo[d]imidazol-1-yl)piperidine-2,6-dione

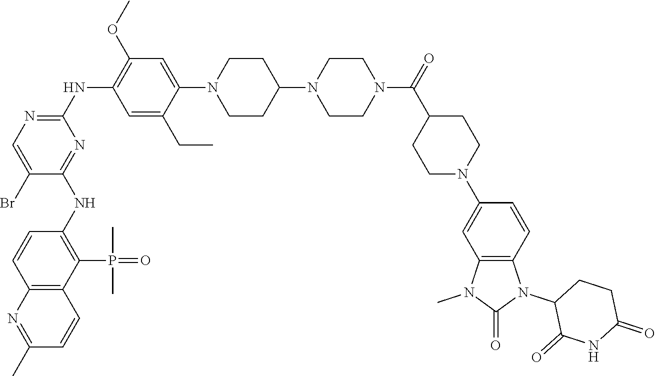 [SMILES: CCc1cc(Nc2ncc(Br)c(Nc3ccc4nc(C)ccc4c3P(C)(C)=O)n2)c(OC)cc1N1CCC(N2CCN(C(=O)C3CCN(c4ccc5c(c4)n(C)c(=O)n5C4CCC(=O)NC4=O)CC3)CC2)CC1]

A mixture of 1-(1-(2,6-dioxopiperidin-3-yl)-3-methyl-2-oxo-2,3-dihydro-1H-benzo[d]imidazol-5-yl)piperidine-4-carboxylic acid (28.5 mg, 0.073 mmol), HATU (23.7 mg, 0.062 mmol) and DIEA (21.9 mg, 0.17 mmol) in DMF (4 mL) was stirred at rt for 10 min. Then, (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (40 mg, 0.056 mmol) was added to the mixture. The resulting mixture was stirred at rt for 16 h. The mixture was diluted with water (50 mL), extracted with EA (3×50 mL). The combined organic layers were dried over Na₂SO₄, filtered and concentrated under vacuum. The residue was purified by Prep-HPLC with C-18 column chromatography (0.1% FA in water: acetonitrile=90: 10˜ 70: 30 gradient elution) to afford the product (9.60 mg, 15.7%). ¹H NMR (500 MHz, DMSO) δ_{H} 11.76 (s, 1H), 11.06 (s, 1H), 8.56 (d, J=10.0 Hz, 1H), 8.34-8.26 (m, 1H), 8.21 (s, 1H), 7.98 (s, 1H), 7.87 (d, J=10.0 Hz, 1H), 7.42 (d, J=10.0 Hz, 1H), 7.38 (s, 1H), 6.92 (d, J=5.0 Hz, 1H), 6.85-6.83 (m, 1H), 6.74 (s, 1H), 6.66-6.62 (m, 1H), 5.32-5.26 (m, 1H), 3.76 (s, 3H), 3.65-3.59 (m, 2H), 3.57-3.51 (m, 2H), 3.50-3.45 (m, 2H), 2.99-2.84 (m, 3H), 2.79-2.52 (m, 16H), 2.44-2.25 (m, 4H), 2.01-1.96 (m, 7H), 1.88-1.78 (m, 2H), 1.75-1.68 (m, 4H), 1.63-1.52 (m, 2H), 0.82-0.71 (m, 3H). [M+H]⁺=1075.4.

### Example 211: 3-(5-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-1H-indazol-1-yl)piperidine-2,6-dione

### Step 1: 3-(5-bromo-1H-indazol-1-yl)-1-(4-methoxybenzyl)piperidine-2,6-dione and 3-(5-bromo-2H-indazol-2-yl)-1-(4-methoxybenzyl)piperidine-2,6-dione

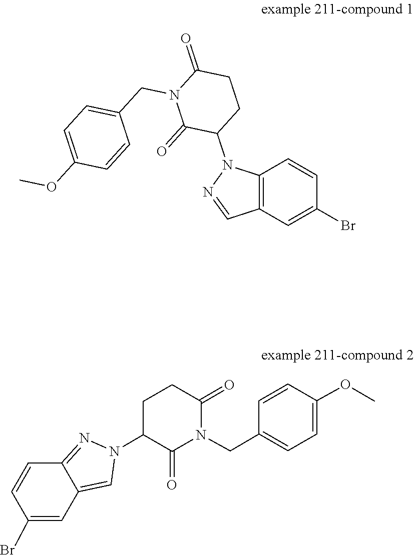 [SMILES: COc1ccc(CN2C(=O)CCC(n3ncc4cc(Br)ccc43)C2=O)cc1 ; COc1ccc(CN2C(=O)CCC(n3cc4cc(Br)ccc4n3)C2=O)cc1]

To a solution of 5-bromo-1H-indazole (2.0 g, 10.2 mmol) and 1-(4-methoxybenzyl)-2,6-dioxopiperidin-3-yl trifluoromethanesulfonate (This intermediate was prepared according to the same manner described in WO2020113233A1) (5.8 g, 15.3 mmol) in THF(50 mL) was added t-BuOK (15 mL, 15.3 mmol) at 0° C.

Then the mixture was stirred at 30° C. for 2 hrs. The reaction was quenched with water and the mixture was extracted with DCM (3×300 mL). The combined organic layers were washed with brine (300 mL), dried over anhydrous Na₂SO₄. After filtration, the filtrate was concentrated under reduced pressure. The residue was purified by silica gel column chromatography, eluted with DCM/MeOH(15: 1) to afford the example 211-compound 1 (1.2 g, 28%). [M+H]⁺=428.2 and example 211-compound 2 (1.3 g, 30%) [M+H]⁺=428.1

### Step 2: 3-(5-bromo-1H-indazol-1-vll)piperidine-2,6-dione

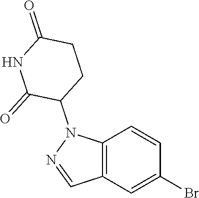 [SMILES: O=C1CCC(n2ncc3cc(Br)ccc32)C(=O)N1]

A mixture of 3-(5-bromo-1H-indazol-1-yl)-1-(4-methoxybenzyl)piperidine-2,6-dione (1.2 g, 2.8 mmol) in toluene (10 mL) and MsOH (5 mL) was stirred in a round bottom flask at 100° C. under nitrogen atmosphere for 3 hours. The reaction mixture was concentrated under reduced pressure. The residue was purified by purified by silica gel column chromatography, eluted with DCM/MeOH (15: 1) to afford the product (480 mg, 56%), [M+H]⁺=308.2.

### Step 3: (E)-3-(5-(2-ethoxyvinyl)-1H-indazol-1-yl)piperidine-2,6-dione

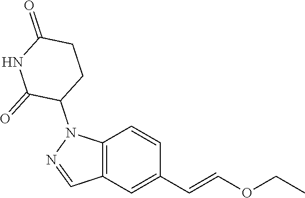 [SMILES: CCO/C=C/c1ccc2c(cnn2C2CCC(=O)NC2=O)c1]

A mixture of 3-(5-bromo-1H-indazol-1-yl)piperidine-2,6-dione (70 mg, 0.23 mmol), (E)-2-(2-ethoxyvinyl)-4,4,5,5-tetramethyl-1,3,2-dioxaborolane (91 mg, 0.46 mmol), Pd(dppf)Cl₂ (15 mg, 0.023 mmol) and CsF (100 mg, 0.69 mmol) in DMF (5 mL) was stirred in a round bottom flask at 90° C. under nitrogen atmosphere for 2 hrs. The reaction mixture was allowed to cool down to room temperature. The resulting mixture was extracted with DCM (3×100 mL). The combined organic layers were washed with brine (100 mL), dried over anhydrous Na₂SO₄. After filtration, the filtrate was concentrated under reduced pressure. The residue was purified by silica gel column chromatography, eluted with DCM/MeOH (15: 1) to afford the product (50 mg, 72%), [M+H]⁺=300.0.

### Step 4: 2-(1-(2,6-dioxopiperidin-3-yl)-1H-indazol-5-yl)acetaldehyde

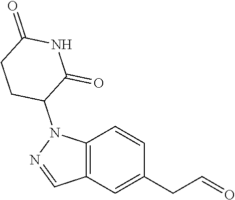 [SMILES: O=CCc1ccc2c(cnn2C2CCC(=O)NC2=O)c1]

A mixture of (E)-2-(2-ethoxyvinyl)-4,4,5,5-tetramethyl-1,3,2-dioxaborolane (50 mg, 0.17 mmol) in HCOOH (2 mL) was stirred in a round bottom flask at 25° C. under nitrogen atmosphere for 30 min. The reaction mixture was concentrated under reduced pressure to afford crude product which was directly used for the next step. [M+H]⁺=272.2.

### Step 5: 3-(5-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-1H-indazol-1-yl)piperidine-2, 6-dione

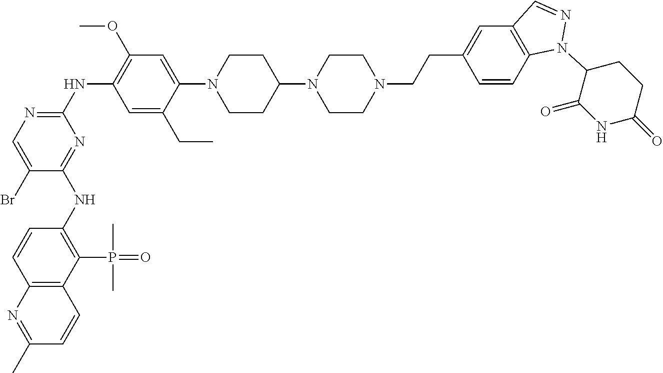 [SMILES: CCc1cc(Nc2ncc(Br)c(Nc3ccc4nc(C)ccc4c3P(C)(C)=O)n2)c(OC)cc1N1CCC(N2CCN(CCc3ccc4c(cnn4C4CCC(=O)NC4=O)c3)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (40 mg, 0.057 mmol) and 2-(1-(2,6-dioxopiperidin-3-yl)-1H-indazol-5-yl)acetaldehyde (20 mg, 0.069 mmol) and NaOAc (14 mg, 0.17 mmol) in DCM (4 mL) and EtOH (0.5 mL) was stirred in a round bottom flask at room temperature for 2 hour. The mixture was added NaBH₃CN (10 mg, 0.17 mmol) and stirred in a round bottom flask at room temperature 2 h. Then the mixture was evaporated in vacuum to afford the crude product, which was purified by HPLC chromatography with C-18 column chromatography (0.1% FA in water: acetonitrile=90: 10˜ 60: 40 gradient elution) to give the product (15 mg, 27%). ¹H NMR (500 MHz, DMSO) δ 11.76 (s, 1H), 11.09 (s, 1H), 8.56 (d, J=8.9 Hz, 1H), 8.30 (s, 1H), 8.21 (s, 1H), 8.14 (s, 1H), 8.05 (s, 1H), 7.98 (s, 1H), 7.87 (d, J=9.1 Hz, 1H), 7.60 (s, 1H), 7.54 (d, J=8.6 Hz, 1H), 7.45-7.36 (m, 2H), 7.31 (d, J=8.7 Hz, 1H), 6.74 (s, 1H), 5.81 (dd, J=11.7, 5.0 Hz, 1H), 3.76 (s, 3H), 2.98-2.90 (m, 2H), 2.89-2.82 (m, 3H), 2.78-2.53 (m, 14H), 2.33-2.40 (m, 5H), 1.98 (d, J=13.3 Hz, 7H), 1.92-1.81 (m, 2H), 1.62-1.50 (m, 2H), 0.81-0.69 (m, 3H); [M+H]⁺=962.5.

### Example 212: 3-(4-(2-(4-(1-(4-((5-bromo-4-((5-(dimethylphosphoryl)quinoxalin-6-yl)amino)pyrimidin-2-yl)amino)-3-methoxyphenyl)piperidin-4-yl)piperazin-1-yl)ethyl)-2-fluorophenyl)piperidine-2,6-dione

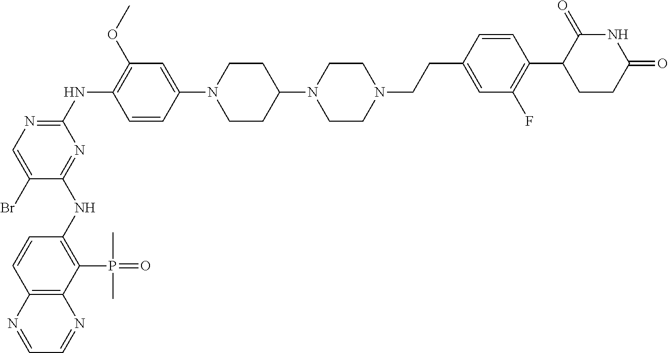 [SMILES: COc1cc(N2CCC(N3CCN(CCc4ccc(C5CCC(=O)NC5=O)c(F)c4)CC3)CC2)ccc1Nc1ncc(Br)c(Nc2ccc3nccnc3c2P(C)(C)=O)n1]

The titled compound was prepared in a manner similar to that in Example 200. ¹H NMR (500 MHz, DMSO) δ 12.65 (d, J=25.5 Hz, 1H), 10.86 (s, 1H), 8.88 (d, J=8.5 Hz, 1H), 8.84 (dd, J=9.7, 1.8 Hz, 2H), 8.27 (d, J=9.5 Hz, 1H), 8.23 (s, 1H), 7.94 (d, J=8.7 Hz, 1H), 7.30 (d, J=8.1 Hz, 1H), 7.20 (t, J=7.9 Hz, 1H), 7.08 (d, J=11.8 Hz, 1H), 7.03 (t, J=8.8 Hz, 1H), 6.67 (d, J=2.0 Hz, 1H), 6.49 (dd, J=8.7, 2.3 Hz, 1H), 4.00 (dd, J=12.5, 4.9 Hz, 1H), 3.83-3.72 (m, 5H), 3.31-3.22 (m, 4H), 2.78-2.67 (m, 5H), 2.54 (m, 4H), 2.36 (m, 4H), 2.24-2.13 (m, 1H), 2.07-1.95 (m, 7H), 1.86 (d, J=10.4 Hz, 2H), 1.52 (m, 2H). [M+H]⁺=899.3

### Example 213: 3-(4-(4-(2-(4-(1-(4-((5-bromo-4-((5-(dimethylphosphoryl)quinoxalin-6-yl)amino)pyrimidin-2-yl)amino)-3-methoxyphenyl)piperidin-4-yl)piperazin-1-yl)ethyl)piperidin-1-yl)-2,6-difluorophenyl)piperidine-2,6-dione

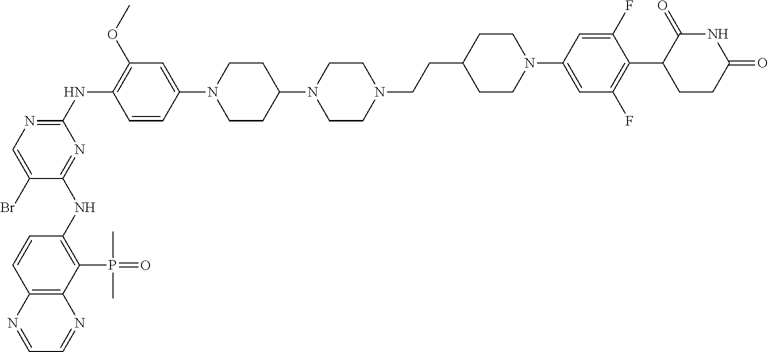 [SMILES: COc1cc(N2CCC(N3CCN(CCC4CCN(c5cc(F)c(C6CCC(=O)NC6=O)c(F)c5)CC4)CC3)CC2)ccc1Nc1ncc(Br)c(Nc2ccc3nccnc3c2P(C)(C)=O)n1]

The titled compound was prepared in a manner similar to that in Example 200. ¹H NMR (500 MHz, DMSO) δ 12.68 (s, 1H), 10.86 (s, 1H), 8.89 (s, 1H), 8.85 (dd, J=9.4, 1.7 Hz, 2H), 8.36 (s, 1H), 8.27 (s, 1H), 7.94 (d, J=9.0 Hz, 1H), 7.30 (d, J=7.8 Hz, 1H), 6.66 (t, J=7.5 Hz, 1H), 6.60 (d, J=12.8 Hz, 2H), 6.48 (dd, J=8.7, 2.2 Hz, 1H), 4.04 (dd, J=12.6, 4.9 Hz, 1H), 3.81-3.70 (m, 7H), 3.26-3.20 (m, 4H), 2.83-2.62 (m, 6H), 2.52 (s, 2H), 2.39-2.25 (m, 6H), 2.13-1.91 (m, 9H), 1.85 (d, J=11.3 Hz, 2H), 1.71 (d, J=11.4 Hz, 2H), 1.50 (t, J=11.2 Hz, 2H), 1.41-1.34 (m, 2H), 1.17 (d, J=12.5 Hz, 1H). [M+H]⁺=1000.3

### Example 214: 3-(4-(2-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)ethyl)-2-fluorophenyl)piperidine-2,6-dione

### Step 1: tert-butyl 4-(1-(5-methoxy-2-methyl-4-nitrophenyl)piperidin-4-yl)piperazine-1-carboxylate

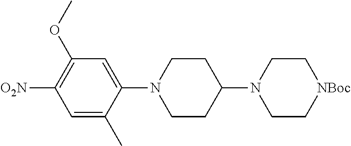 [SMILES: COc1cc(N2CCC(N3CCN(C(=O)OC(C)(C)C)CC3)CC2)c(C)cc1[N+](=O)[O-]]

The titled compound (840 mg, 86%) was prepared in a manner similar to that in Example 216 step 3 from 1-fluoro-5-methoxy-2-methyl-4-nitrobenzene and tert-butyl 4-(piperidin-4-yl)piperazine-1-carboxylate. [M+H]⁺=435.3

### Step 2: tert-butyl 4-(1-(4-amino-5-methoxy-2-methylphenyl)piperidin-4-yl)piperazine-1-carboxylate

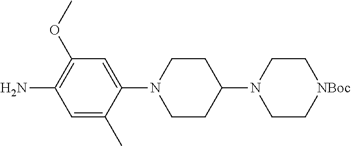 [SMILES: COc1cc(N2CCC(N3CCN(C(=O)OC(C)(C)C)CC3)CC2)c(C)cc1N]

The titled compound (720 mg, 88%) was prepared in a manner similar to that in Example 216 step 4 from tert-butyl 4-(1-(5-methoxy-2-methyl-4-nitrophenyl)piperidin-4-yl)piperazine-1-carboxylate and Pd/C. [M+H]⁺=405.3

### Step 3: (6-((5-bromo-2-((2-methoxy-5-methyl-4-(4-(piperazin-1-yl)piperidin-1-yl)phenyl)amino)pyrimidin-4-yl)amino)quinoxalin-5-yl)dimethylphosphine oxide

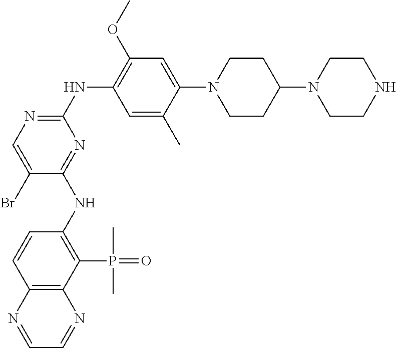 [SMILES: COc1cc(N2CCC(N3CCNCC3)CC2)c(C)cc1Nc1ncc(Br)c(Nc2ccc3nccnc3c2P(C)(C)=O)n1]

The titled compound (420 mg, 67%) was prepared in a manner similar to that in Example 216 step 5 from tert-butyl 4-(1-(4-amino-5-methoxy-2-methylphenyl)piperidin-4-yl)piperazine-1-carboxylate and (6-((5-bromo-2-chloropyrimidin-4-yl)amino)quinoxalin-5-yl)dimethylphosphine oxide. [M+H]⁺=680.2.

### Step 4: 3-(4-(2-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)ethyl)-2-fluorophenyl)piperidine-2,6-dione

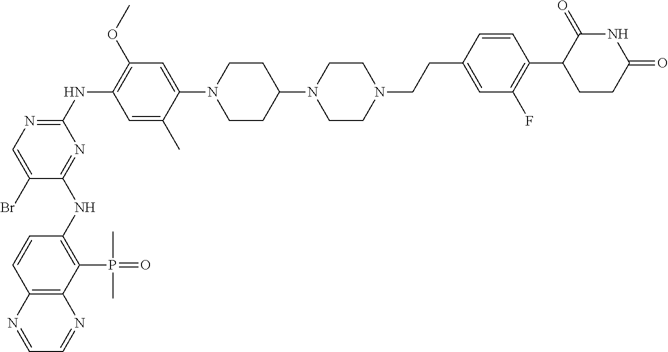 [SMILES: COc1cc(N2CCC(N3CCN(CCc4ccc(C5CCC(=O)NC5=O)c(F)c4)CC3)CC2)c(C)cc1Nc1ncc(Br)c(Nc2ccc3nccnc3c2P(C)(C)=O)n1]

The titled compound (50 mg, 36%) was prepared in a manner similar to that in Example 204 step 6 from (6-((5-bromo-2-((2-methoxy-5-methyl-4-(4-(piperazin-1-yl)piperidin-1-yl)phenyl)amino)pyrimidin-4-yl)amino)quinoxalin-5-yl)dimethylphosphine oxide and 2-(4-(2,6-dioxopiperidin-3-yl)-3-fluorophenyl)acetaldehyde. ¹H NMR (500 MHz, DMSO) δ 12.68 (s, 1H), 10.86 (s, 1H), 8.86 (dd, J=8.3, 1.9 Hz, 3H), 8.26 (d, J=9.0 Hz, 2H), 7.93 (d, J=9.6 Hz, 1H), 7.36 (s, 1H), 7.20 (t, J=7.9 Hz, 1H), 7.08 (d, J=11.7 Hz, 1H), 7.05 (d, J=7.8 Hz, 1H), 6.75 (s, 1H), 4.00 (dd, J=12.5, 4.9 Hz, 1H), 3.77 (s, 3H), 3.10 (d, J=11.2 Hz, 2H), 2.79-2.71 (m, 3H), 2.67 (m, 3H), 2.55 (m, 8H), 2.38-2.32 (m, 1H), 2.19 (dt, J=12.9, 8.7 Hz, 1H), 2.08 (d, J=9.6 Hz, 5H), 2.05-1.97 (m, 7H), 1.88 (d, J=11.0 Hz, 2H), 1.59 (m, 2H). [M+H]⁺=913.3.

### Example 215: 3-(4-(4-(2-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-1-yl)ethyl)piperidin-1-yl)-2,6-difluorophenyl)piperidine-2,6-dione

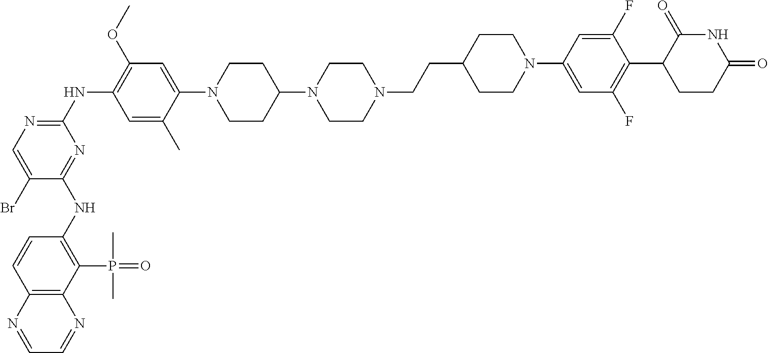 [SMILES: COc1cc(N2CCC(N3CCN(CCC4CCN(c5cc(F)c(C6CCC(=O)NC6=O)c(F)c5)CC4)CC3)CC2)c(C)cc1Nc1ncc(Br)c(Nc2ccc3nccnc3c2P(C)(C)=O)n1]

The titled compound was prepared in a manner similar to that in Example 200. ¹H NMR (500 MHz, DMSO) δ 12.69 (s, 1H), 10.86 (s, 1H), 8.86 (dd, J=8.6, 1.8 Hz, 3H), 8.27 (s, 1H), 8.25 (s, 1H), 7.92 (d, J=9.2 Hz, 1H), 7.36 (s, 1H), 6.75 (s, 1H), 6.62 (s, 1H), 6.59 (s, 1H), 4.04 (dd, J=12.5, 5.0 Hz, 1H), 3.77 (s, 3H), 3.74 (d, J=12.6 Hz, 2H), 3.31-3.29 (m, 4H), 3.09 (d, J=11.0 Hz, 2H), 2.73 (m, 5H), 2.54 (s, 2H), 2.43-2.27 (m, 6H), 2.13-2.06 (m, 4H), 2.02 (s, 6H), 1.99-1.92 (m, 1H), 1.87 (d, J=11.3 Hz, 2H), 1.73 (t, J=11.7 Hz, 2H), 1.58 (m, 2H), 1.48 (s, 1H), 1.42-1.34 (m, 2H), 1.24-1.13 (m, 2H). [M+H]⁺=1014.4

### Example 216: 3-(4-(4-(2-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)-3-oxopiperazin-1-yl)ethyl)piperidin-1-yl)-2,6-difluorophenyl)piperidine-2,6-dione

### Step 1: tert-butyl 4-(1-((benzyloxy)carbonyl)-1,2,3,6-tetrahydropyridin-4-yl)-3-oxopiperazine-1-carboxylate

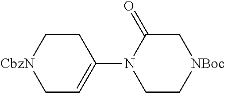 [SMILES: CC(C)(C)OC(=O)N1CCN(C2=CCN(C(=O)OCc3ccccc3)CC2)C(=O)C1]

To a solution of tert-butyl 3-oxopiperazine-1-carboxylate (3.5 g, 17.4 mmol), benzyl 4-(4,4,5,5-tetramethyl-1,3-dioxolan-2-yl)-3,6-dihydropyridine-1(2H)-carboxylate (4.0 g, 11.6 mmol) and CuI (2.2 g, 11.6 mmol) in DMSO (40 mL) was stirred at 60° C. for 16 hrs. The mixture was extracted with EtOAc (100.0 mL). The combined organic phase was washed with brine (100.0 mL×3), dried over Na₂SO₄, filtered and concentrated in vacuum. The residue was purified by column chromatography (PE/EA=10/1 to 1/1) to afford product (4.2 g, 87.3%). [M+H]⁺=416.2

### Step 2: tert-butyl 3-oxo-4-(piperidin-4-yl)piperazine-1-carboxylate

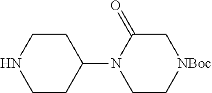 [SMILES: CC(C)(C)OC(=O)N1CCN(C2CCNCC2)C(=O)C1]

To a solution of tert-butyl 4-(1-((benzyloxy)carbonyl)-1,2,3,6-tetrahydropyridin-4-yl)-3-oxopiperazine-1-carboxylate (4.2 g, 10.1 mmol) and Pd/C (2.1 g) in DCM (30 mL) and MeOH (30 mL) was stirred at 35° C. for overnight. The mixture was filtered and concentrated in vacuum to afford product (2.6 g, 92.3%). [M+H]⁺=284.2

### Step 3: tert-butyl 4-(1-(5-methoxy-2-methyl-4-nitrophenyl)piperidin-4-yl)-3-oxopiperazine-1-carboxylate

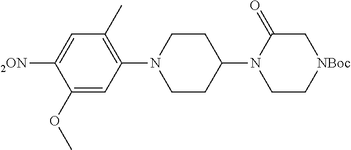

To a solution of 1-fluoro-5-methoxy-2-methyl-4-nitrobenzene (1 g, 5.4 mmol), tert-butyl 3-oxo-4-(piperidin-4-yl)piperazine-1-carboxylate (1.8 g, 6.5 mmol) and K₂CO₃ (1.5 g, 10.8 mmol) in DMF (40 mL) was stirred at 80° C. for overnight. The mixture was extracted with EtOAc (100.0 mL). The combined organic phase was washed with brine (100.0 mL×3), dried over Na₂SO₄, filtered and concentrated in vacuum. The residue was purified by column chromatography (PE/EA=10/1 to 1/1) to afford product (1.3 g, 53.7%). [M+H]⁺=449.2

### Step 4: tert-butyl 4-(1-(4-amino-5-methoxy-2-methylphenyl)piperidin-4-yl)-3-oxopiperazine-1-carboxylate

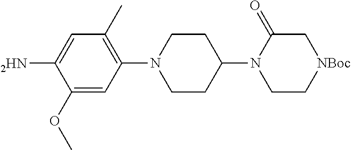

To a solution of tert-butyl 4-(1-(5-methoxy-2-methyl-4-nitrophenyl)piperidin-4-yl)-3-oxopiperazine-1-carboxylate (1.3 g, 2.9 mmol) and Pd/C (400 mg) in DCM (20 mL) and MeOH (20 mL) was stirred at 35° C. for 16 hrs under hydrogen atmosphere. The mixture was filtered and concentrated in vacuum to afford product (1.0 g, 82.3%). [M+H]⁺=419.2.

### Step 5: 1-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-2-one

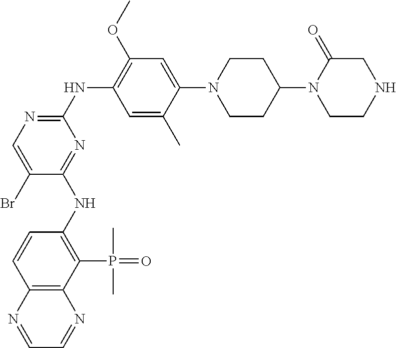 [SMILES: COc1cc(N2CCC(N3CCNCC3=O)CC2)c(C)cc1Nc1ncc(Br)c(Nc2ccc3nccnc3c2P(C)(C)=O)n1]

The titled compound (50 mg, 36%) was prepared in a manner similar to that in Example 203 step 3 from tert-butyl 4-(1-(4-amino-5-methoxy-2-methylphenyl)piperidin-4-yl)-3-oxopiperazine-1-carboxylate and (6-((5-bromo-2-chloropyrimidin-4-yl)amino)quinoxalin-5-yl)dimethylphosphine oxide. [M+H]⁺=694.2

### Step 6: 3-(4-(4-(2-(4-(1-(4-((5-bromo-4-((5-(dimethylphosphoryl)quinoxalin-6-yl)amino)pyrimidin-2-yl)amino)-5-methoxy-2-methylphenyl)piperidin-4-yl)-3-oxopiperazin-1-yl)ethyl)piperidin-1-yl)-2,6-difluorophenyl)piperidine-2,6-dione

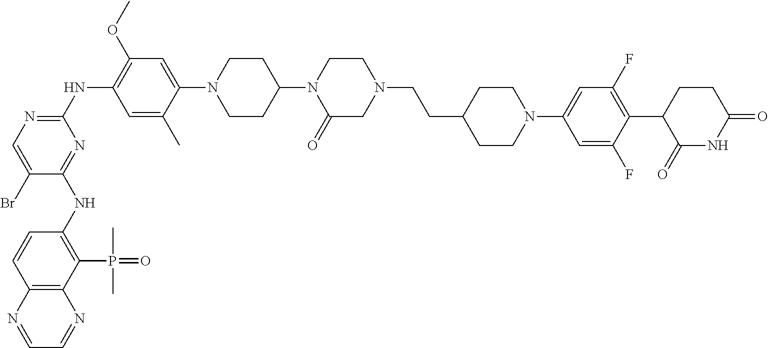 [SMILES: COc1cc(N2CCC(N3CCN(CCC4CCN(c5cc(F)c(C6CCC(=O)NC6=O)c(F)c5)CC4)CC3=O)CC2)c(C)cc1Nc1ncc(Br)c(Nc2ccc3nccnc3c2P(C)(C)=O)n1]

The titled compound (11 mg, 26%) was prepared in a manner similar to that in Example 204 step 6 from 1-(1-(4-((5-bromo-4-((5-(dimethylphosphoryl)quinoxalin-6-yl)amino)pyrimidin-2-yl)amino)-5-methoxy-2-methylphenyl)piperidin-4-yl)piperazin-2-one and 2-(1-(4-(2,6-dioxopiperidin-3-yl)-3,5-difluorophenyl)piperidin-4-yl)acetaldehyde. ¹H NMR (500 MHz, DMSO) δ 12.69 (s, 1H), 10.86 (s, 1H), 8.86 (dd, J=7.1, 1.8 Hz, 3H), 8.27 (s, 2H), 7.94 (d, J=9.6 Hz, 1H), 7.36 (s, 1H), 6.81 (s, 1H), 6.62 (s, 1H), 6.60 (s, 1H), 4.39 (d, J=12.0 Hz, 1H), 4.04 (dd, J=12.4, 4.8 Hz, 1H), 3.79 (s, 3H), 3.74 (d, J=12.6 Hz, 2H), 3.28-3.20 (m, 4H), 3.11 (d, J=10.9 Hz, 2H), 3.01 (s, 2H), 2.75 (m, 4H), 2.63 (d, J=1.6 Hz, 2H), 2.54 (s, 1H), 2.38 (dd, J=13.2, 4.4 Hz, 2H), 2.09 (m, 4H), 2.02 (s, 6H), 1.97-1.83 (m, 3H), 1.73 (d, J=11.6 Hz, 2H), 1.62 (d, J=10.9 Hz, 2H), 1.50 (s, 1H), 1.44-1.36 (m, 2H), 1.24-1.13 (m, 2H). [M+H]⁺=1028.3

### Example 217: 3-(4-(4-(2-(4-(1-(4-((5-bromo-4-((5-(dimethylphosphoryl)-2-methylquinolin-6-yl)amino)pyrimidin-2-yl)amino)-5-methoxy-2-methylphenyl) piperidin-4-yl)piperazin-1-yl)ethyl)piperidin-1-yl)-2,6-difluorophenyl)piperidine-2,6-dione

### Step 1: (6-((5-bromo-2-((2-methoxy-5-methyl-4-(4-(piperazin-1-yl)piperidin-1-yl)phenyl)amino)pyrimidin-4-yl)amino)-2-methylquinolin-5-yl)dimethylphosphine oxide

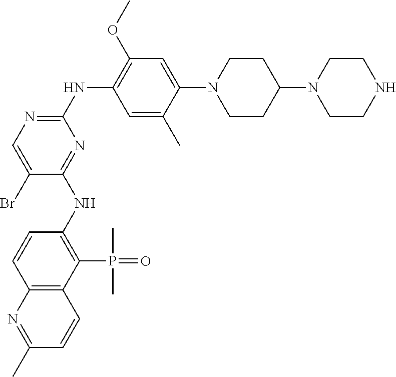 [SMILES: COc1cc(N2CCC(N3CCNCC3)CC2)c(C)cc1Nc1ncc(Br)c(Nc2ccc3nc(C)ccc3c2P(C)(C)=O)n1]

The titled compound (62 mg, 32%) was prepared in a manner similar to that in Example 203 step 3 from tert-butyl 4-(1-(4-amino-5-methoxy-2-methylphenyl)piperidin-4-yl)piperazine-1-carboxylate and (6-((5-bromo-2-chloropyrimidin-4-yl)amino)-2-methylquinolin-5-yl)dimethylphosphine oxide. [M+H]⁺=693.2.

### Step 2: 3-(4-(4-(2-(4-(1-(4-((5-bromo-4-((5-(dimethylphosphoryl)-2-methylquinolin-6-yl)amino)pyrimidin-2-yl)amino)-5-methoxy-2-methylphenyl)piperidin-4-yl)piperazin-1-yl)ethyl)piperidin-1-yl)-2,6-difluorophenyl)piperidine-2,6-dione

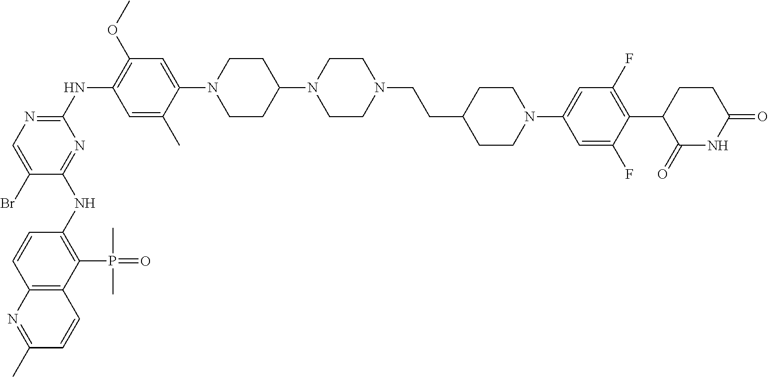 [SMILES: COc1cc(N2CCC(N3CCN(CCC4CCN(c5cc(F)c(C6CCC(=O)NC6=O)c(F)c5)CC4)CC3)CC2)c(C)cc1Nc1ncc(Br)c(Nc2ccc3nc(C)ccc3c2P(C)(C)=O)n1]

The titled compound (11 mg, 22%) was prepared in a manner similar to that in Example 204 step 6 from (6-((5-bromo-2-((2-methoxy-5-methyl-4-(4-(piperazin-1-yl)piperidin-1-yl)phenyl)amino)pyrimidin-4-yl)amino)-2-methylquinolin-5-yl)dimethylphosphine oxide and 2-(1-(4-(2,6-dioxopiperidin-3-yl)-3,5-difluorophenyl)piperidin-4-yl)acetaldehyde. ¹H NMR (500 MHz, DMSO) δ 11.91 (s, 1H), 10.86 (s, 1H), 8.51 (d, J=8.8 Hz, 1H), 8.38 (d, J=5.9 Hz, 1H), 8.20 (s, 1H), 7.96 (s, 1H), 7.88 (d, J=9.3 Hz, 1H), 7.42 (d, J=8.9 Hz, 1H), 7.34 (s, 1H), 6.68 (s, 1H), 6.62 (s, 1H), 6.59 (s, 1H), 4.04 (dd, J=12.5, 5.0 Hz, 1H), 3.74 (m, 5H), 3.21 (m, 4H), 3.02 (d, J=10.9 Hz, 2H), 2.80-2.53 (m, 11H), 2.34 (m, 6H), 2.08 (dt, J=13.6, 9.6 Hz, 1H), 1.99 (s, 6H), 1.92 (s, 3H), 1.84 (d, J=10.8 Hz, 2H), 1.71 (d, J=11.7 Hz, 2H), 1.53 (m, 3H), 1.45-1.34 (m, 2H), 1.25-1.11 (m, 2H). [M+H]⁺=1027.4

### Example 218: 3-(4-(4-(2-(4-(1-(4-((5-bromo-4-((5-(dimethylphosphoryl)-2-methylquinolin-6-yl)amino)pyrimidin-2-yl)amino)-3-methoxyphenyl)piperidin-4-yl)piperazin-1-yl)ethyl)piperidin-1-yl)-2,6-difluorophenyl)piperidine-2, 6-dione

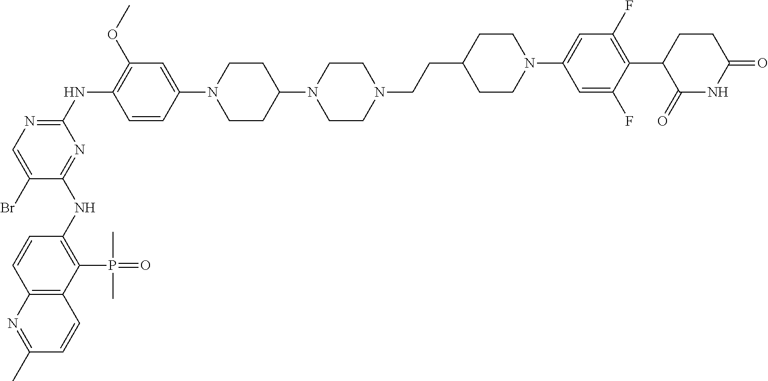 [SMILES: COc1cc(N2CCC(N3CCN(CCC4CCN(c5cc(F)c(C6CCC(=O)NC6=O)c(F)c5)CC4)CC3)CC2)ccc1Nc1ncc(Br)c(Nc2ccc3nc(C)ccc3c2P(C)(C)=O)n1]

The titled compound was prepared in a manner similar to that in Example 217. ¹H NMR (500 MHz, DMSO) δ 11.99 (s, 1H), 10.86 (s, 1H), 8.45 (t, J=11.1 Hz, 2H), 8.17 (s, 1H), 7.99 (s, 1H), 7.90 (d, J=9.2 Hz, 1H), 7.41 (d, J=8.9 Hz, 1H), 7.26 (s, 1H), 6.62 (s, 1H), 6.59 (s, 2H), 6.26 (d, J=8.2 Hz, 1H), 4.04 (dd, J=12.6, 5.0 Hz, 1H), 3.74 (m, 5H), 3.66 (d, J=12.0 Hz, 2H), 3.28-3.25 (m, 2H), 2.82-2.60 (m, 9H), 2.54 (s, 2H), 2.32 (m, 7H), 2.08 (dt, J=13.6, 9.8 Hz, 1H), 1.97 (m, 7H), 1.83 (d, J=11.4 Hz, 2H), 1.71 (d, J=12.2 Hz, 2H), 1.49 (m, 3H), 1.42-1.33 (m, 2H), 1.17 (q, J=11.4 Hz, 2H). [M+H]⁺=1013.4.

### Example 220: 3-(4-((R)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)pyrrolidin-1-yl)phenyl)piperidine-2,6-dione

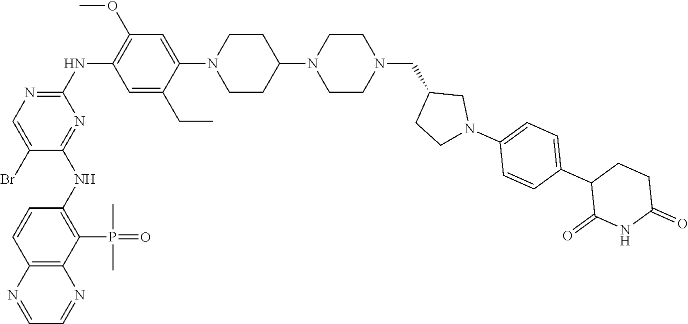 [SMILES: CCc1cc(Nc2ncc(Br)c(Nc3ccc4nccnc4c3P(C)(C)=O)n2)c(OC)cc1N1CCC(N2CCN(C[C@H]3CCN(c4ccc(C5CCC(=O)NC5=O)cc4)C3)CC2)CC1]

The titled compound was prepared in a manner similar to that in Example 207. 1H NMR (400 MHz, DMSO) δ 12.65 (s, 1H), 10.74 (s, 1H), 8.86 (d, J=4.0 Hz, 2H), 8.81 (s, 1H), 8.27 (s, 2H), 7.90 (d, J=9.6 Hz, 1H), 7.38 (s, 1H), 6.99 (d, J=8.5 Hz, 2H), 6.81 (s, 1H), 6.48 (d, J=8.6 Hz, 2H), 3.77 (s, 3H), 3.68 (dd, J=10.4, 4.9 Hz, 1H), 3.35 (m, 1H), 3.28-3.24 (m, 2H), 3.20 (d, J=7.8 Hz, 1H), 3.02 (d, J=10.8 Hz, 2H), 2.98-2.92 (m, 1H), 2.71 (t, J=10.9 Hz, 2H), 2.63 (m, 1H), 2.58 (m, 5H), 2.46 (m, 3H), 2.42 (m, 2H), 2.33 (d, J=6.8 Hz, 3H), 2.07 (d, J=6.7 Hz, 2H), 2.02 (d, J=14.4 Hz, 8H), 1.87 (d, J=11.7 Hz, 2H), 1.70 (s, 1H), 1.59 (d, J=8.8 Hz, 2H), 0.93 (t, J=7.2 Hz, 3H); [M+H]⁺=964.7.

### Example 221: 3-(4-((S)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)pyrrolidin-1-yl)phenyl)piperidine-2,6-dione

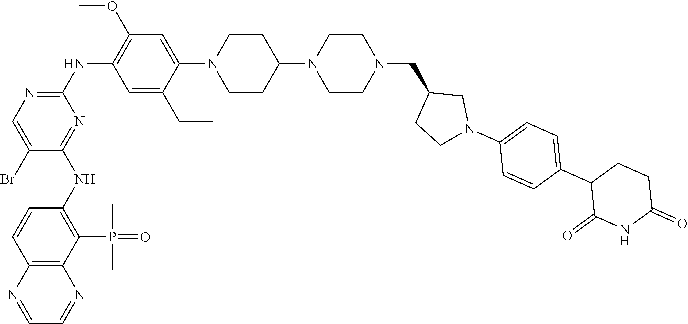 [SMILES: CCc1cc(Nc2ncc(Br)c(Nc3ccc4nccnc4c3P(C)(C)=O)n2)c(OC)cc1N1CCC(N2CCN(C[C@@H]3CCN(c4ccc(C5CCC(=O)NC5=O)cc4)C3)CC2)CC1]

The titled compound was prepared in a manner similar to that in Example 207. 1H NMR (400 MHz, DMSO) δ 12.64 (s, 1H), 10.74 (s, 1H), 8.86 (d, J=4.3 Hz, 2H), 8.82 (s, 1H), 8.27 (s, 2H), 7.90 (d, J=8.9 Hz, 1H), 7.38 (s, 1H), 6.99 (d, J=8.4 Hz, 2H), 6.81 (s, 1H), 6.48 (d, J=8.4 Hz, 2H), 3.77 (s, 3H), 3.68 (dd, J=10.2, 4.9 Hz, 1H), 3.20 (d, J=7.5 Hz, 4H), 3.02 (d, J=10.4 Hz, 3H), 2.96 (d, J=6.8 Hz, 2H), 2.71 (t, J=11.5 Hz, 3H), 2.59 (s, 5H), 2.48-2.42 (m, 4H), 2.35 (s, 2H), 2.08 (s, 2H), 2.02 (d, J=14.3 Hz, 8H), 1.88 (d, J=10.7 Hz, 2H), 1.71 (d, J=7.2 Hz, 1H), 1.59 (d, J=8.9 Hz, 2H), 0.93 (t, J=7.0 Hz, 3H); [M+H]⁺=964.8.

### Example 222: 5-(3-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-2-oxoethyl)azetidin-1-yl)-2-(2,6-dioxopiperidin-3-yl)isoindoline-1,3-dione

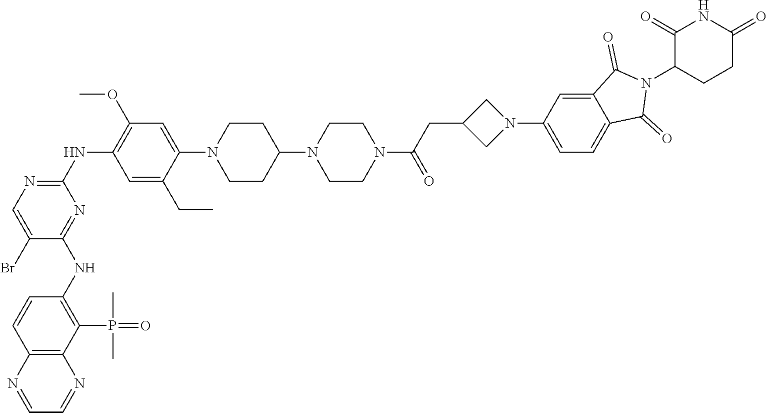 [SMILES: CCc1cc(Nc2ncc(Br)c(Nc3ccc4nccnc4c3P(C)(C)=O)n2)c(OC)cc1N1CCC(N2CCN(C(=O)CC3CN(c4ccc5c(c4)C(=O)N(C4CCC(=O)NC4=O)C5=O)C3)CC2)CC1]

The titled compound was prepared in a manner similar to that in Example 57. 1H NMR (400 MHz, DMSO) δ 12.65 (s, 1H), 11.08 (s, 1H), 8.86 (dd, J=8.9, 1.9 Hz, 3H), 8.28 (d, J=10.0 Hz, 2H), 7.91 (s, 1H), 7.63 (d, J=8.3 Hz, 1H), 7.37 (s, 1H), 6.82-6.74 (m, 2H), 6.63 (dd, J=8.4, 2.0 Hz, 1H), 5.05 (dd, J=12.7, 5.4 Hz, 1H), 4.18 (t, J=8.2 Hz, 2H), 3.77 (s, 3H), 3.71-3.66 (m, 2H), 3.45 (s, 4H), 3.02 (d, J=11.8 Hz, 3H), 2.87 (d, J=11.7 Hz, 1H), 2.79 (d, J=7.7 Hz, 2H), 2.71 (t, J=11.2 Hz, 2H), 2.60 (s, 1H), 2.57-2.52 (m, 3H), 2.48 (s, 3H), 2.36 (s, 2H), 2.02 (d, J=14.4 Hz, 7H), 1.85 (d, J=10.8 Hz, 2H), 1.60 (d, J=8.6 Hz, 2H), 0.92 (t, J=7.2 Hz, 3H); [M+H]⁺=1047.4.

### Example 223: 3-(4-((S)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione

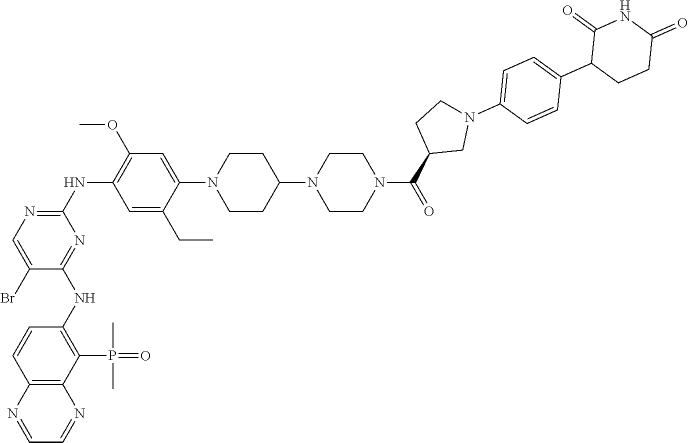 [SMILES: CCc1cc(Nc2ncc(Br)c(Nc3ccc4nccnc4c3P(C)(C)=O)n2)c(OC)cc1N1CCC(N2CCN(C(=O)[C@H]3CCN(c4ccc(C5CCC(=O)NC5=O)cc4)C3)CC2)CC1]

The titled compound was prepared in a manner similar to that in Example 208. 1H NMR (400 MHz, DMSO) δ 12.64 (s, 1H), 10.75 (s, 1H), 8.87 (d, J=4.8 Hz, 3H), 8.28 (d, J=5.9 Hz, 2H), 7.90 (d, J=9.0 Hz, 1H), 7.38 (s, 1H), 7.00 (d, J=8.4 Hz, 2H), 6.82 (s, 1H), 6.51 (d, J=8.5 Hz, 2H), 3.77 (s, 3H), 3.69 (d, J=6.0 Hz, 1H), 3.58 (s, 3H), 3.48 (d, J=22.9 Hz, 7H), 3.29-3.24 (m, 4H), 3.03 (d, J=10.2 Hz, 2H), 2.72 (t, J=11.0 Hz, 2H), 2.58 (s, 3H), 2.43 (s, 2H), 2.12 (d, J=8.2 Hz, 2H), 2.02 (d, J=14.3 Hz, 7H), 1.87 (d, J=10.0 Hz, 2H), 1.61 (d, J=9.3 Hz, 2H), 0.94 (d, J=7.0 Hz, 3H); [M+H]⁺=978.8.

### Example 224: 3-(4-((R)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione

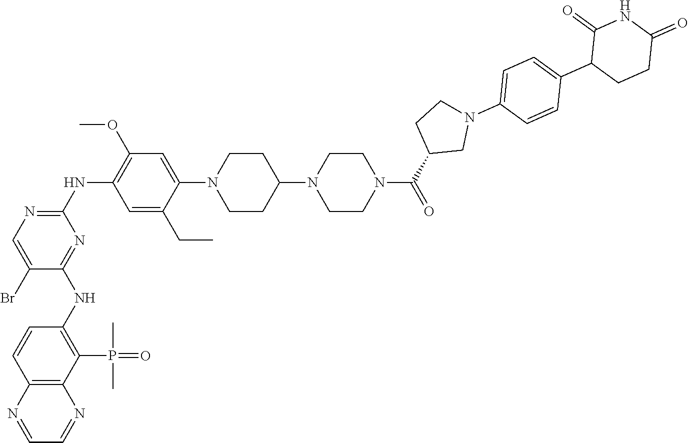 [SMILES: CCc1cc(Nc2ncc(Br)c(Nc3ccc4nccnc4c3P(C)(C)=O)n2)c(OC)cc1N1CCC(N2CCN(C(=O)[C@@H]3CCN(c4ccc(C5CCC(=O)NC5=O)cc4)C3)CC2)CC1]

The titled compound was prepared in a manner similar to that in Example 208. 1H NMR (400 MHz, DMSO) δ 12.64 (s, 1H), 10.75 (s, 1H), 8.87 (d, J=4.6 Hz, 3H), 8.28 (d, J=5.5 Hz, 2H), 7.90 (d, J=9.1 Hz, 1H), 7.38 (s, 1H), 7.00 (d, J=8.3 Hz, 2H), 6.82 (s, 1H), 6.51 (d, J=8.5 Hz, 2H), 3.77 (s, 3H), 3.69 (dd, J=10.8, 5.1 Hz, 1H), 3.58 (s, 2H), 3.46 (dd, J=19.8, 11.6 Hz, 5H), 3.25 (d, J=7.2 Hz, 4H), 3.03 (d, J=9.9 Hz, 2H), 2.72 (t, J=11.1 Hz, 2H), 2.60 (d, J=14.8 Hz, 3H), 2.48 (s, 2H), 2.43 (s, 2H), 2.13 (dd, J=14.9, 6.9 Hz, 2H), 2.02 (d, J=14.4 Hz, 8H), 1.87 (d, J=9.8 Hz, 2H), 1.61 (d, J=8.6 Hz, 2H), 0.93 (t, J=7.2 Hz, 3H); [M+H]⁺=978.8.

### Example 225: 3-(3-((S)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione

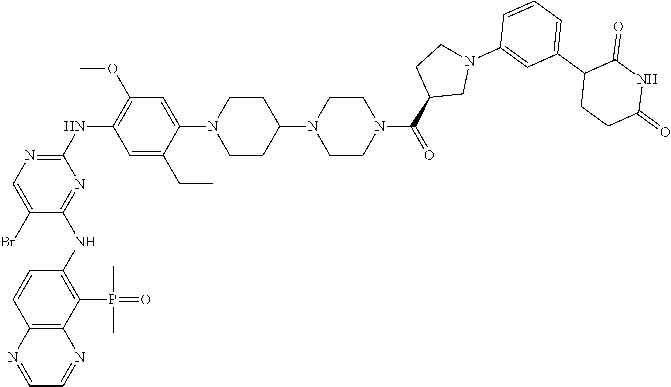 [SMILES: CCc1cc(Nc2ncc(Br)c(Nc3ccc4nccnc4c3P(C)(C)=O)n2)c(OC)cc1N1CCC(N2CCN(C(=O)[C@H]3CCN(c4cccc(C5CCC(=O)NC5=O)c4)C3)CC2)CC1]

The titled compound was prepared in a manner similar to that in Example 208. 1H NMR (400 MHz, DMSO) δ 12.65 (s, 1H), 10.81 (s, 1H), 8.85 (dd, J=16.2, 14.6 Hz, 3H), 8.31 (s, 1H), 8.28 (s, 1H), 7.90 (d, J=8.4 Hz, 1H), 7.37 (s, 1H), 7.11 (t, J=7.8 Hz, 1H), 6.82 (s, 1H), 6.45 (d, J=7.8 Hz, 2H), 6.39 (s, 1H), 3.77 (s, 3H), 3.76-3.73 (m, 1H), 3.59-3.50 (m, 6H), 3.44 (s, 3H), 3.29-3.22 (m, 3H), 3.04 (d, J=10.0 Hz, 2H), 2.73 (t, J=11.0 Hz, 2H), 2.67-2.60 (m, 2H), 2.48 (s, 2H), 2.45 (d, J=4.6 Hz, 1H), 2.19-2.15 (m, 2H), 2.10 (s, 1H), 2.03 (t, J=10.8 Hz, 8H), 1.88 (s, 2H), 1.63 (s, 2H), 0.93 (t, J=7.2 Hz, 3H); [M+H]⁺=978.3.

### Example 226: 3-(3-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)phenyl)piperidine-2,6-dione

### Step 1: (1-(3-(2,6-bis(benzyloxy)pyridin-3-yl)phenyl)azetidin-3-yl)methanol

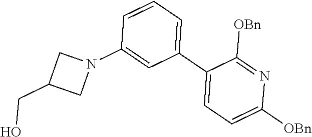 [SMILES: OCC1CN(c2cccc(-c3ccc(OCc4ccccc4)nc3OCc3ccccc3)c2)C1]

A mixture of 2,6-bis(benzyloxy)-3-(3-bromophenyl)pyridine (500 mg, 1.12 mmol), azetidin-3-ylmethanol hydrochloride (206 mg, 1.68 mmol), CuI (21 mg, 0.112 mmol), L-Proline (26 mg, 0.224 mmol) and K₃PO₄ (712 mg, 3.36 mmol) in DMSO (8 mL) was stirred in a round bottom flask at 120° C. under nitrogen atmosphere for 16 hrs. The reaction mixture was allowed to cool down to room temperature. The resulting mixture was extracted with EtOAc (3×40 mL). The combined organic layers were washed with water (3×40 mL) and brine (100 mL), dried over anhydrous Na₂SO₄. After filtration, the filtrate was concentrated under reduced pressure. The residue was purified by silica gel column chromatography, eluted with MeOH/DCM (0˜5%) to afford the product (305 mg, 60.2%). [M+H]⁺=453.1.

### Step 2: 3-(3-(3-(hydroxymethyl)azetidin-1-yl)phenyl)piperidine-2,6-dione

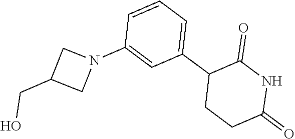 [SMILES: O=C1CCC(c2cccc(N3CC(CO)C3)c2)C(=O)N1]

To a stirred solution of (1-(3-(2,6-bis(benzyloxy)pyridin-3-yl)phenyl)azetidin-3-yl)methanol (300 mg, 0.664 mmol) in MeOH (5 mL) and DCM (5 mL) was added Pd/C (wet, 10%) (150 mg). The resulting mixture was stirred for 16 hrs at room temperature under hydrogen atmosphere. The resulting mixture was filtered, the filter cake was washed with DCM/CH₃OH (10: 1, 20 mL). The filtrate was concentrated under reduced pressure to afford the product (155 mg, 85.6%). [M+H]⁺=275.1.

### Step 3: (1-(3-(2,6-dioxopiperidin-3-yl)phenyl)azetidin-3-yl)methyl methanesulfonate

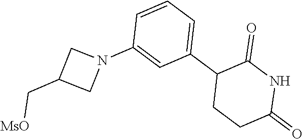 [SMILES: CS(=O)(=O)OCC1CN(c2cccc(C3CCC(=O)NC3=O)c2)C1]

The titled compound (83 mg, 43.2%) was prepared in a manner similar to that in Example 207 step 4 from 3-(3-(3-(hydroxymethyl)azetidin-1-yl)phenyl)piperidine-2,6-dione and methanesulfonyl chloride. [M+H]⁺=353.2

### Step 4: 3-(3-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)phenyl)piperidine-2,6-dione

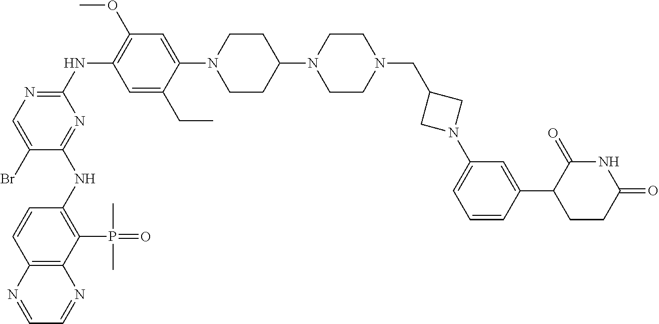 [SMILES: CCc1cc(Nc2ncc(Br)c(Nc3ccc4nccnc4c3P(C)(C)=O)n2)c(OC)cc1N1CCC(N2CCN(CC3CN(c4cccc(C5CCC(=O)NC5=O)c4)C3)CC2)CC1]

The titled compound (9.3 mg, 13.7%) was prepared in a manner similar to that in Example 207 step 5 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and (1-(3-(2,6-dioxopiperidin-3-yl)phenyl)azetidin-3-yl)methyl methanesulfonate. 1H NMR (400 MHz, DMSO) δ 12.65 (s, 1H), 10.80 (s, 1H), 8.87 (d, J=5.9 Hz, 2H), 8.82 (s, 1H), 8.28 (d, J=4.4 Hz, 2H), 7.90 (d, J=9.2 Hz, 1H), 7.38 (s, 1H), 7.10 (t, J=7.8 Hz, 1H), 6.81 (s, 1H), 6.50 (d, J=7.8 Hz, 1H), 6.31 (d, J=8.1 Hz, 1H), 6.25 (s, 1H), 3.90 (d, J=3.2 Hz, 2H), 3.78-3.71 (m, 4H), 3.42 (d, J=5.7 Hz, 2H), 3.01 (d, J=10.7 Hz, 2H), 2.91 (s, 1H), 2.71 (t. J=11.2 Hz, 2H), 2.63-2.58 (m, 2H), 2.56-2.53 (m, 4H), 2.48-2.45 (m, 3H), 2.44-2.41 (m, 4H), 2.32-2.30 (m, 2H), 2.16-2.13 (m, 1H), 2.02 (d, J=14.4 Hz, 7H), 1.86 (d, J=11.6 Hz, 2H), 1.57 (d, J=9.3 Hz, 2H), 0.93 (t, J=7.2 Hz, 3H); [M+H]⁺=950.5.

### Example 227: 3-(6-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)-1-oxoisoindolin-2-yl)piperidine-2,6-dione

### Step 1: 1-(4-cyano-3-(methoxycarbonyl)phenyl)azetidine-3-carboxylic acid

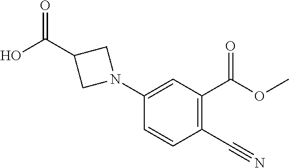 [SMILES: COC(=O)c1cc(N2CC(C(=O)O)C2)ccc1C#N]

To a stirred mixture of methyl 2-cyano-5-fluorobenzoate (10.0 g, 55.82 mmol) and 3-azetidinecarboxylic acid (6.8 g, 66.98 mmol) in DMSO (100.00 mL) was added DIEA (14.4 g, 111.638 mmol) and stirred overnight at 60° C. The resulting mixture was extracted with EtOAc (500 mL). The combined organic layers were washed with citric acid (1000 mL), dried over anhydrous Na₂SO₄. After filtration, the filtrate was concentrated under reduced pressure to afford the product (11.0 g, 75.7%). [M+H]⁺=261.2.

### Step 2: 1-(4-formyl-3-(methoxycarbonyl)phenyl)azetidine-3-carboxylic acid

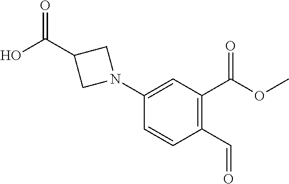 [SMILES: COC(=O)c1cc(N2CC(C(=O)O)C2)ccc1C=O]

To a stirred mixture of 1-(4-cyano-3-(methoxycarbonyl)phenyl)azetidine-3-carboxylic acid (6.0 g, 23.055 mmol) in AcOH (60 mL) and H₂O (30 mL) was added Raney-Ni (6.0 g, 70.032 mmol) in portions and stirred overnight at room temperature under air atmosphere. The resulting mixture was filtered, the filter cake was washed with DCM and MeOH (300 mL). The filtrate was concentrated under reduced pressure. The filtrate was extracted with EtOAc (300 mL). The combined organic layers were washed with citric acid (1M in water, 300 mL), dried over anhydrous Na₂SO₄. After filtration, the filtrate was concentrated under reduced pressure.

The residue was purified by silica gel column chromatography, eluted with CH₂Cl₂/MeOH (9:1) to afford the product (5.0 g, 82.3%) [M+H]⁺=264.3.

### Step 3: 1-(2-(2,6-dioxopiperidin-3-yl)-3-oxoisoindolin-5-yl)azetidine-3-carboxylic acid

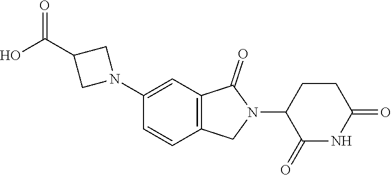 [SMILES: O=C1CCC(N2Cc3ccc(N4CC(C(=O)O)C4)cc3C2=O)C(=O)N1]

A mixture of 3-aminopiperidine-2,6-dione hydrochloride (9.4 g, 56.980 mmol) and DIEA (9.8 g, 75.974 mmol) in DMF (60 mL) was stirred for 5 h at room temperature. The mixture was acidified to pH<7 with AcOH (11.4 g, 189.934 mmol) followed by the addition of 1-(4-formyl-3-(methoxycarbonyl)phenyl)azetidine-3-carboxylic acid (5.0 g, 18.993 mmol) in DMF (10 mL) at room temperature. The resulting mixture was stirred overnight at room temperature. To the mixture was added NaBH₃CN (3.6 g, 56.980 mmol) in portions at room temperature. The resulting mixture was stirred for additional 2 h at room temperature. The reaction was quenched with water (50 mL) at room temperature. The resulting mixture was concentrated under reduced pressure. The residue was purified by silica gel column chromatography, eluted with CH₂Cl₂/MeOH (5:1 v/v, 0.2 v % AcOH) to afford crude product, which was purified by trituration with CH₂Cl₂ (40 mL). 1-(2-(2,6-dioxopiperidin-3-yl)-3-oxoisoindolin-5-yl)azetidine-3-carboxylic acid (2.1 g, 32.7%) was obtained. [M+H]⁺=344.2.

### Step 3: 3-(6-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)-1-oxoisoindolin-2-yl)piperidine-2,6-dione

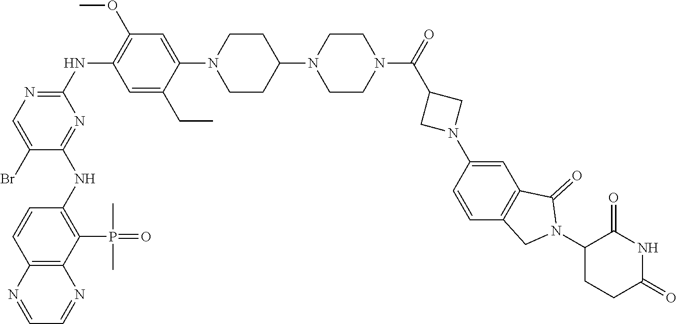 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc5c(c4)C(=O)N(C4CCC(=O)NC4=O)C5)C3)CC2)CC1]

The titled compound (11 mg, 26%) was prepared in a manner similar to that in Example 208 step 8 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 1-(2-(2,6-dioxopiperidin-3-yl)-3-oxoisoindolin-5-yl)azetidine-3-carboxylic acid. 1H NMR (400 MHz, DMSO) δ 12.65 (s, 1H), 10.97 (s, 1H), 8.88-8.85 (m, 2H), 8.82 (s, 1H), 8.28 (d, J=8.8 Hz, 2H), 7.90 (d, J=9.5 Hz, 1H), 7.42-7.36 (m, 2H), 6.82 (s, 1H), 6.73 (d, J=7.6 Hz, 2H), 5.09 (dd, J=13.3, 5.1 Hz, 1H), 4.32 (d, J=16.6 Hz, 1H), 4.20 (d, J=16.5 Hz, 1H), 4.08 (s, 2H), 3.93 (s, 2H), 3.87-3.82 (m, 1H), 3.77 (s, 3H), 3.50-3.45 (m, 2H), 3.36-3.31 (m, 2H), 3.30 (s, 1H), 3.03 (d, J=11.3 Hz, 2H), 2.91-2.87 (m, 1H), 2.72 (t, J=11.3 Hz, 2H), 2.62-2.58 (m, 1H), 2.55-2.52 (m, 3H), 2.48-2.46 (m, 2H), 2.38 (m, 2H), 2.02 (d, J=14.4 Hz, 7H), 1.86-1.82 (m, 2H), 1.61-1.58 (m, 2H), 0.93 (t, J=7.2 Hz, 3H); [M+H]⁺=1019.5.

### Example 228: 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)-2-fluorophenyl)piperidine-2,6-dione

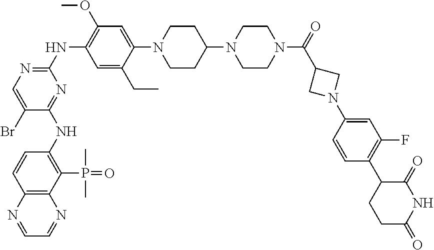 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc(C5CCC(=O)NC5=O)c(F)c4)C3)CC2)CC1]

The titled compound was prepared in a manner similar to that in Example 208. 1H NMR (400 MHz, DMSO) δ 12.65 (s, 1H), 10.80 (s, 1H), 8.87 (dd, J=7.9, 1.9 Hz, 2H), 8.81 (s, 1H), 8.28 (d, J=8.8 Hz, 2H), 7.90 (d, J=7.8 Hz, 1H), 7.37 (s, 1H), 7.06 (t, J=8.3 Hz, 1H), 6.81 (s, 1H), 6.25 (dd, J=15.3, 7.2 Hz, 2H), 4.02 (s, 2H), 3.88 (m, 5H), 3.77 (s, 3H), 3.50 (s, 2H), 3.30 (s, 1H), 3.02 (d, J=10.8 Hz, 2H), 2.72-2.68 (m, 3H), 2.54-2.53 (m, 2H), 2.52-2.50 (m, 1H), 2.48-2.43 (m, 3H), 2.41-2.38 (m, 1H), 2.13-2.09 (m, 1H), 2.02 (d, J=14.4 Hz, 7H), 1.95-1.91 (m, 1H), 1.85 (d, J=9.8 Hz, 2H), 1.61 (d, J=8.8 Hz, 2H), 0.93 (t, J=7.2 Hz, 3H); [M+H]⁺=982.5.

### Example 230: 3-(3-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)phenyl)piperidine-2,6-dione

### Step 1: methyl 1-(3-(2,6-bis(benzyloxy)pyridin-3-yl)phenyl)azetidine-3-carboxylate

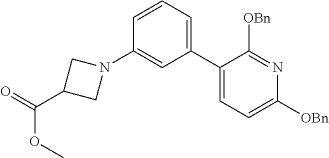 [SMILES: COC(=O)C1CN(c2cccc(-c3ccc(OCc4ccccc4)nc3OCc3ccccc3)c2)C1]

A mixture of 2,6-bis(benzyloxy)-3-(3-bromophenyl)pyridine (500 mg, 1.12 mmol), methyl azetidine-3-carboxylate hydrochloride (254 mg, 1.68 mmol), CuI (21 mg, 0.112 mmol), L-Proline (26 mg, 0.224 mmol) and K₃PO₄ (712 mg, 3.36 mmol) in DMSO (8 mL) was stirred in a round bottom flask at 120° C. under nitrogen atmosphere for 16 hrs. The reaction mixture was allowed to cool down to room temperature. The resulting mixture was extracted with EtOAc (3×40 mL). The combined organic layers were washed with water (3×40 mL) and brine (100 mL), dried over anhydrous Na₂SO₄. After filtration, the filtrate was concentrated under reduced pressure. The residue was purified by silica gel column chromatography, eluted with MeOH/DCM (0˜5%) to afford the product (210 mg, 39.2%). [M+H]⁺=481.1.

### Step 2: 1-(3-(2,6-bis(benzyloxy)pyridin-3-yl)phenyl)azetidine-3-carboxylic acid

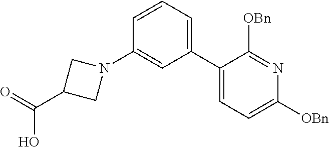 [SMILES: O=C(O)C1CN(c2cccc(-c3ccc(OCc4ccccc4)nc3OCc3ccccc3)c2)C1]

To a solution of methyl 1-(3-(2,6-bis(benzyloxy)pyridin-3-yl)phenyl)azetidine-3-carboxylate (200 mg, 0.417 mmol) in THF (5 mL) and H₂O (1 mL) was added lithium hydroxide hydrate (26 mg, 0.625 mmol) at 25° C. The resulting mixture was stirred at 25° C. for 12 h. The reaction was quenched with HCl (1 N) at 0° C. until PH=5 and extracted with DCM (2×10 mL). The combined organic layers were washed with brine, dried over anhydrous Na₂SO₄, and evaporated in vacuum to afford the crude product (155 mg, 79.9%), which was used for next step without further purification. [M+H]⁺=467.2.

### Step 3: 1-(3-(2,6-dioxopiperidin-3-yl)phenyl)azetidine-3-carboxylic acid

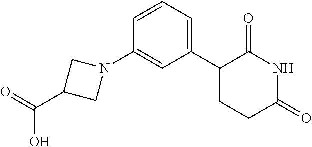 [SMILES: O=C1CCC(c2cccc(N3CC(C(=O)O)C3)c2)C(=O)N1]

To a stirred solution of 1-(3-(2,6-bis(benzyloxy)pyridin-3-yl)phenyl)azetidine-3-carboxylic acid (150 mg, 0.322 mmol) in MeOH (3 mL) and DCM (3 mL) was added Pd/C (wet, 10%) (100 mg). The resulting mixture was stirred for 16 hrs at room temperature under hydrogen atmosphere. The resulting mixture was filtered, the filter cake was washed with DCM/CH₃OH (10: 1, 10 mL). The filtrate was concentrated under reduced pressure to afford the product (68 mg, 73.9%). [M+H]⁺=289.1.

### Step 4: 3-(3-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)phenyl)piperidine-2,6-dione

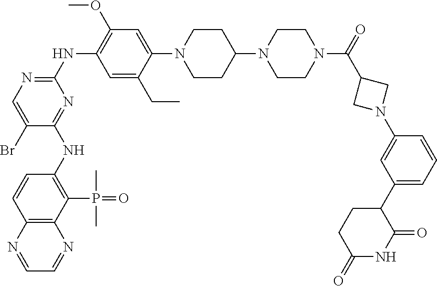 [SMILES: CCc1cc(Nc2ncc(Br)c(Nc3ccc4nccnc4c3P(C)(C)=O)n2)c(OC)cc1N1CCC(N2CCN(C(=O)C3CN(c4cccc(C5CCC(=O)NC5=O)c4)C3)CC2)CC1]

The titled compound (7.4 mg, 27.6%) was prepared in a manner similar to that in Example 208 step 8 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 1-(3-(2,6-dioxopiperidin-3-yl)phenyl)azetidine-3-carboxylic acid. 1H NMR (400 MHz, DMSO) δ 12.65 (s, 1H), 10.80 (s, 1H), 8.87 (dd, J=8.0, 1.8 Hz, 2H), 8.82 (s, 1H), 8.28 (d, J=8.4 Hz, 2H), 7.89 (s, 1H), 7.37 (s, 1H), 7.12 (t, J=7.8 Hz, 1H), 6.81 (s, 1H), 6.54 (d, J=7.6 Hz, 1H), 6.36 (d, J=8.2 Hz, 1H), 6.31 (s, 1H), 4.01 (s, 2H), 3.84 (m, 3H), 3.77 (s, 3H), 3.76-3.73 (m, 1H), 3.49 (s, 1H), 3.31 (s, 3H), 3.02 (d, J=10.3 Hz, 2H), 2.72 (t, J=11.0 Hz, 2H), 2.68-2.59 (m, 2H), 2.54 (s, 2H), 2.48-2.42 (m, 2H), 2.45-2.41 (m, 1H), 2.38-2.35 (m, 1H), 2.18 (d, J=8.9 Hz, 1H), 2.02 (d, J=14.4 Hz, 8H), 1.85 (d, J=10.8 Hz, 2H), 1.60 (d, J=10.8 Hz, 2H), 0.93 (t, J=7.2 Hz, 3H); [M+H]⁺=964.5.

### Example 231: 3-(4-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)phenyl)piperidine-2,6-dione

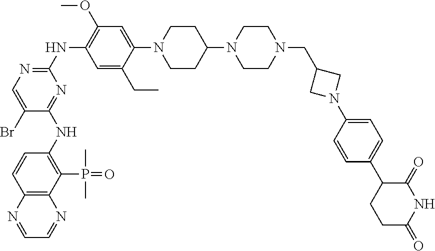 [SMILES: CCc1cc(Nc2ncc(Br)c(Nc3ccc4nccnc4c3P(C)(C)=O)n2)c(OC)cc1N1CCC(N2CCN(CC3CN(c4ccc(C5CCC(=O)NC5=O)cc4)C3)CC2)CC1]

The titled compound was prepared in a manner similar to that in Example 207. 1H NMR (400 MHz, DMSO) δ 13.07 (s, 1H), 11.18 (s, 1H), 9.29 (dd, J=7.3, 1.8 Hz, 2H), 9.24 (s, 1H), 8.70 (d, J=6.4 Hz, 2H), 8.33 (d, J=9.4 Hz, 1H), 7.80 (s, 1H), 7.42 (d, J=8.5 Hz, 2H), 7.24 (s, 1H), 6.80 (d, J=8.5 Hz, 2H), 4.33 (t, J=7.4 Hz, 2H), 4.19 (s, 3H), 4.12 (dd, J=10.9, 4.9 Hz, 1H), 3.87-3.83 (m, 4H), 3.44 (d, J=10.8 Hz, 2H), 3.32-3.31 (m, 1H), 3.13 (t, J=11.0 Hz, 2H), 3.07-3.01 (m, 2H), 2.98 (d, J=7.3 Hz, 4H), 2.88-2.85 (m, 3H), 2.82-2.79 (m, 2H), 2.73 (s, 1H), 2.54-2.51 (m, 1H), 2.43 (t, J=13.4 Hz, 8H), 2.28 (d, J=11.1 Hz, 2H), 1.99 (d, J=9.0 Hz, 2H), 1.35 (t, J=7.0 Hz, 3H); [M+H]⁺=950.7.

### Example 232: 5-((R)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)pyrrolidin-1-yl)-2-(2,6-dioxopiperidin-3-yl)isoindoline-1,3-dione

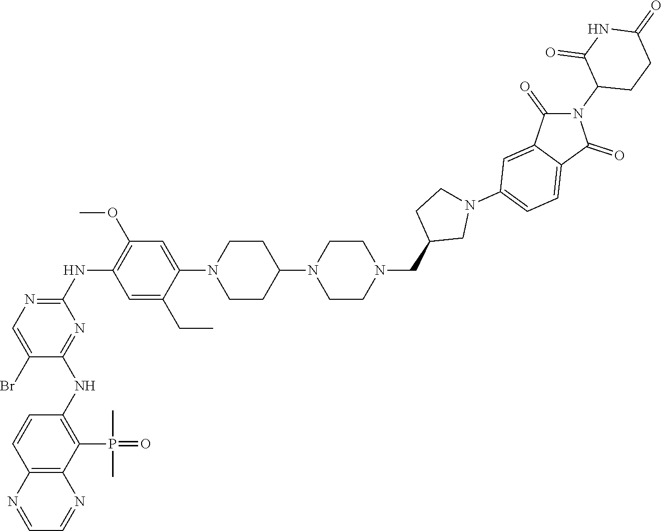 [SMILES: CCc1cc(Nc2ncc(Br)c(Nc3ccc4nccnc4c3P(C)(C)=O)n2)c(OC)cc1N1CCC(N2CCN(C[C@H]3CCN(c4ccc5c(c4)C(=O)N(C4CCC(=O)NC4=O)C5=O)C3)CC2)CC1]

The titled compound was prepared in a manner similar to that in Example 59. 1H NMR (400 MHz, DMSO) δ 12.64 (s, 1H), 11.06 (s, 1H), 8.87 (dd, J=7.4, 1.9 Hz, 2H), 8.82 (s, 1H), 8.27 (s, 2H), 7.90 (d, J=9.1 Hz, 1H), 7.64 (d, J=8.4 Hz, 1H), 7.38 (s, 1H), 6.90 (d, J=1.8 Hz, 1H), 6.83-6.80 (m, 2H), 5.05 (dd, J=12.9, 5.4 Hz, 1H), 3.77 (s, 3H), 3.57-3.53 (m, 1H), 3.50 (s, 1H), 3.40 (dd, J=18.0, 7.8 Hz, 2H), 3.30-3.27 (m, 1H), 3.16-3.12 (m, 1H), 3.02 (d, J=10.7 Hz, 2H), 2.92-2.85-2.81 (m, 1H), 2.71 (t, J=10.9 Hz, 2H), 2.64-2.54 (m, 8H), 2.47 (d, J=7.4 Hz, 2H), 2.35 (d, J=7.2 Hz, 3H), 2.15-2.11 (m, 1H), 2.02 (d, J=14.4 Hz, 8H), 1.87 (d, J=11.3 Hz, 2H), 1.78-1.74 (m, 1H), 1.58 (dd, J=20.3, 11.1 Hz, 2H), 0.93 (t, J=7.1 Hz, 3H); [M+H]⁺=1033.7.

### Example 233: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2,5-difluorophenyl)piperidine-2,6-dione

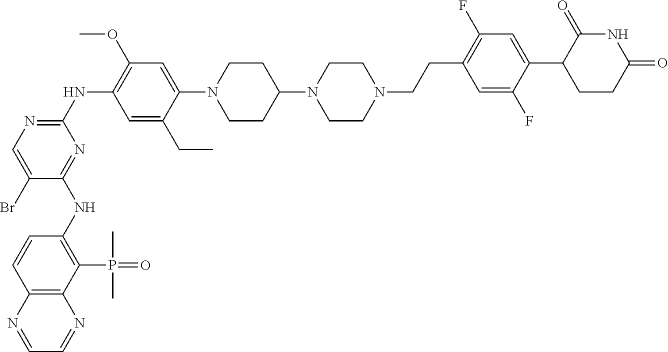 [SMILES: CCc1cc(Nc2ncc(Br)c(Nc3ccc4nccnc4c3P(C)(C)=O)n2)c(OC)cc1N1CCC(N2CCN(CCc3cc(F)c(C4CCC(=O)NC4=O)cc3F)CC2)CC1]

The titled compound was prepared in a manner similar to that in Example 200. 1H NMR (400 MHz, DMSO) δ 12.64 (s, 1H), 10.90 (s, 1H), 8.86 (dd, J=7.2, 1.9 Hz, 2H), 8.82 (s, 1H), 8.27 (s, 2H), 7.90 (d, J=9.6 Hz, 1H), 7.38 (s, 1H), 7.23 (dd, J=10.5, 6.2 Hz, 1H), 7.16 (dd, J=10.1, 6.1 Hz, 1H), 6.81 (s, 1H), 4.03 (dd, J=12.8, 4.7 Hz, 1H), 3.77 (s, 3H), 3.39 (s, 2H), 3.01 (d, J=10.9 Hz, 2H), 2.78-2.68 (m, 6H), 2.58-2.53 (m, 7H), 2.47 (d, J=7.1 Hz, 2H), 2.36-2.32 (m, 1H), 2.25-2.21 (m, 1H), 2.00 (dd, J=17.6, 9.8 Hz, 8H), 1.86 (d, J=11.3 Hz, 2H), 1.58 (dd, J=20.5, 11.0 Hz, 2H), 0.93 (t, J=7.0 Hz, 3H); [M+H]⁺=945.6.

### Example 234: 3-(5-((S)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)pyrrolidin-1-yl)-1-oxoisoindolin-2-yl)piperidine-2,6-dione

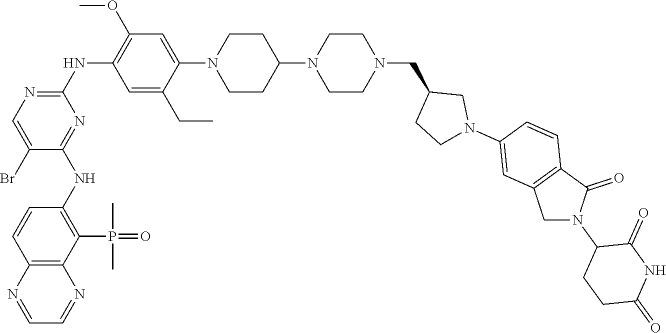 [SMILES: CCc1cc(Nc2ncc(Br)c(Nc3ccc4nccnc4c3P(C)(C)=O)n2)c(OC)cc1N1CCC(N2CCN(C[C@@H]3CCN(c4ccc5c(c4)CN(C4CCC(=O)NC4=O)C5=O)C3)CC2)CC1]

The titled compound was prepared in a manner similar to that in Example 237. 1H NMR (400 MHz, DMSO) δ 12.65 (s, 1H), 10.93 (s, 1H), 8.86 (dd, J=7.5, 1.8 Hz, 2H), 8.82 (s, 1H), 8.27 (s, 2H), 7.90 (d, J=9.2 Hz, 1H), 7.48 (d, J=8.3 Hz, 1H), 7.38 (s, 1H), 6.81 (s, 1H), 6.62 (d, J=10.1 Hz, 2H), 5.03 (dd, J=13.2, 5.1 Hz, 1H), 4.30 (dd, J=16.7, 3.5 Hz, 1H), 4.18 (dd, J=16.7, 2.9 Hz, 1H), 3.77 (s, 3H), 3.46-3.42 (m, 3H), 3.08-3.00 (m, 3H), 2.91-2.87 (m, 1H), 2.71 (t, J=11.0 Hz, 2H), 2.60-2.56 (m, 7H), 2.47 (d, J=7.3 Hz, 2H), 2.41-2.28 (m, 6H), 2.11 (dd, J=11.9, 5.0 Hz, 1H), 2.02 (d, J=14.4 Hz, 7H), 1.98-1.93 (m, 1H), 1.87 (d, J=10.6 Hz, 2H), 1.73 (dd, J=12.1, 7.6 Hz, 1H), 1.58 (dd, J=19.9, 11.2 Hz, 2H), 0.93 (t, J=7.0 Hz, 3H); [M+H]⁺=1019.7.

### Example 235: 5-((S)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)pyrrolidin-1-yl)-2-(2,6-dioxopiperidin-3-yl)isoindoline-1,3-dione

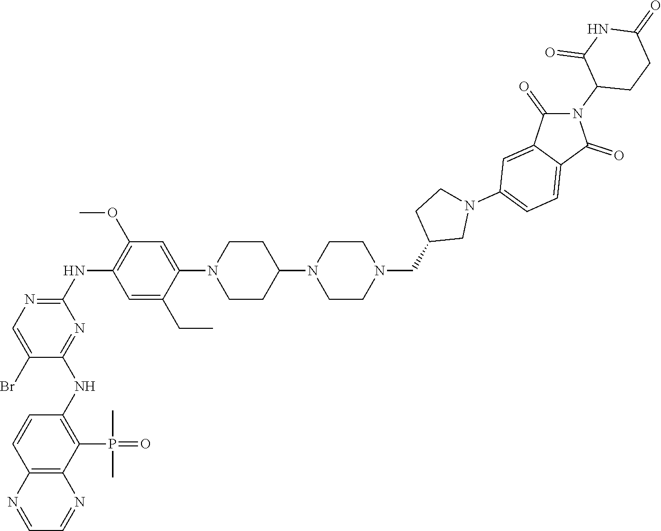 [SMILES: CCc1cc(Nc2ncc(Br)c(Nc3ccc4nccnc4c3P(C)(C)=O)n2)c(OC)cc1N1CCC(N2CCN(C[C@@H]3CCN(c4ccc5c(c4)C(=O)N(C4CCC(=O)NC4=O)C5=O)C3)CC2)CC1]

The titled compound was prepared in a manner similar to that in Example 59. 1H NMR (400 MHz, DMSO) δ 12.64 (s, 1H), 11.06 (s, 1H), 8.87 (dd, J=7.7, 1.8 Hz, 2H), 8.82 (s, 1H), 8.27 (s, 2H), 7.91 (d, J=8.6 Hz, 1H), 7.64 (d, J=8.4 Hz, 1H), 7.38 (s, 1H), 6.90 (s, 1H), 6.82 (d, J=4.1 Hz, 2H), 5.05 (dd, J=12.9, 5.4 Hz, 1H), 3.77 (s, 3H), 3.56-3.53 (m, 3H), 3.40 (d, J=9.7 Hz, 3H), 3.30 (s, 2H), 3.16-3.12 (m, 1H), 3.02 (d, J=10.8 Hz, 2H), 2.88 (t, J=13.1 Hz, 1H), 2.71 (t, J=11.0 Hz, 2H), 2.65-2.56 (m, 6H), 2.54 (d, J=2.9 Hz, 1H), 2.47 (d, J=7.3 Hz, 1H), 2.36 (s, 2H), 2.13 (d, J=6.2 Hz, 1H), 2.02 (d, J=14.4 Hz, 8H), 1.88 (d, J=9.3 Hz, 2H), 1.76 (dd, J=12.1, 7.5 Hz, 1H), 1.59 (d, J=9.8 Hz, 2H), 0.93 (t, J=6.9 Hz, 3H); [M+H]⁺=1033.6.

### Example 236: 5-(((1r,3r)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)cyclobutyl)(methyl)amino)-2-(2,6-dioxopiperidin-3-yl)isoindoline-1,3-dione

### Step 1: tert-butyl (1r,3r)-3-((tert-butoxycarbonyl)amino)cyclobutane-1-carboxylate

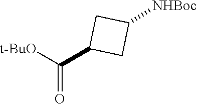 [SMILES: CC(C)(C)OC(=O)N[C@H]1C[C@H](C(=O)OC(C)(C)C)C1]

To a solution of (1r,3r)-tert-butyl 3-aminocyclobutanecarboxylate (45.0 g, 263 mmol) in ACN (270 mL) was added TEA (39.9 g, 394 mmol, 54.9 mL) at 0° C. Boc₂O (63.1 g, 289 mmol, 66.4 mL) was added to the reaction mixture at 0° C. Then the mixture was stirred at 25° C. for 3 hrs. TLC (Petroleum ether/Ethyl aetate=5/1) showed the reaction was completed. The reaction mixture was quenched by addition H₂O (50.0 mL) at 20° C., and then diluted with H₂O (400 mL) and extracted with EA (400 mL×3). The combined organic layers were washed with sat. NaCl aq. (400 mL×2), dried over Na₂SO₄, filtered and concentrated under reduced pressure to give a residue. The residue was purified by column chromatography (SiO₂, Petroleum ether/Ethyl acetate=50/1 to 0/1) to obtain the product (70.0 g, 97%). [M−55]⁺=216.3.

### Step 2: tert-butyl (1r,3r)-3-((tert-butoxycarbonyl)(methyl)amino)cyclobutane-1-carboxylate

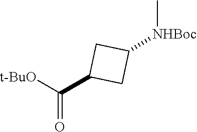

To a solution of NaH (2.65 g, 66.3 mmol, 60%) in DMF (90.0 mL) was added tert-butyl (1r,3r)-3-((tert-butoxycarbonyl)amino)cyclobutane-1-carboxylate (15.0 g, 55.3 mmol) at 0° C. The mixture was stirred at 25° C. for 0.5 hr. Mel (11.8 g, 82.9 mmol, 5.16 mL) was added to the reaction mixture at 0° C. Then the mixture was stirred at 25° C. for 3 hrs. TLC showed (Petroleum ether/Ethyl aetate=5/1) showed the reaction was completed. The reaction mixture was quenched by adding H₂O (20.0 mL) at 0° C., and then diluted with H₂O (100 mL) which was extracted with EA (150 mL×2). The combined organic layers were washed with sat. aq. NaCl solution (100 mL×2), dried over Na₂SO₄, filtered and concentrated under reduced pressure to give a residue. The residue was purified by column chromatography (SiO₂, Petroleum ether/Ethyl acetate=50/1 to 0/1). tert-butyl (1r,3r)-3-((tert-butoxycarbonyl)(methyl)amino)cyclobutane-1-carboxylate (26.0 g, 91.1 mmol, 82.4%) was obtained. [M-55]⁺=230.1.

### Step 3: tert-butyl (1r,3r)-3-(methylamino)cyclobutane-1-carboxylate

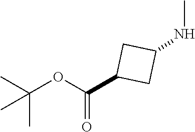

To a solution of tert-butyl (1r,3r)-3-((tert-butoxycarbonyl)(methyl)amino)cyclobutane-1-carboxylate (8.00 g, 28.0 mmol) in DCM (56.0 mL) was added TFA (12.3 g, 108 mmol, 8.00 mL) at 0° C. The mixture was stirred at 25° C. for 3 hrs. TLC showed (Dichloromethane: Methanol=10/1) the reaction was completed. The reaction mixture was quenched by adding sat. aq. NaHCO₃ solution (100 mL) at 0° C., and then diluted with DCM (150 mL×5) which was washed with sat. aq. Na₂CO₃ solution (50.0 mL×3). The combined organic layers were dried over Na₂SO₄, filtered and concentrated under reduced pressure to obtain the product (6 g, 89%). [M−55]⁺=184.2.

### Step 4: 5-fluoroisobenzofuran-1,3-dione

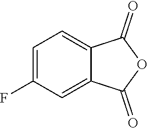 [SMILES: O=C1OC(=O)c2cc(F)ccc21]

To a solution of 4-fluorophthalic acid (10.0 g, 54.3 mmol) in Ac₂O (50 mL) was stirred at 80° C. for 3 hrs. LCMS showed the starting material was consumed completely. The reaction mixture was poured into ice water (40.0 mL), then extracted with EtOAc (30.0 mL×2), the combined organic layer was washed with brine (30.0 mL), dried over Na₂SO₄, filtered and filtrate was concentrated under vacuum. 5-fluoroisobenzofuran-1,3-dione (8.50 g, 94.2% yield) was obtained and used into the next step without further purification. [M+H]⁺=167.2.

### Step 5: 2-(2,6-dioxopiperidin-3-yl)-5-fluoroisoindoline-1,3-dione

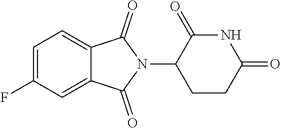 [SMILES: O=C1CCC(N2C(=O)c3ccc(F)cc3C2=O)C(=O)N1]

To a solution of 5-fluoroisobenzofuran-1,3-dione (8.50 g, 51.1 mmol) in HOAc (42.5 mL) was added NaOAc (4.62 g, 56.2 mmol) and 3-aminopiperidine-2,6-dione hydrochloride (8.42 g, 51.1 mmol, 1.00 eq, HCl) at 25° C. The mixture was stirred at 120° C. for 12 hrs. LCMS showed the starting material was consumed completely. The reaction mixture was concentrated under reduced pressure to remove most acetic acid. The residue was poured into water (100 mL) and stirred for 10 minutes. The mixture was filtered, the filtered cake was washed with water (100 mL×2) and dried to give the crude product. 2-(2,6-dioxopiperidin-3-yl)-5-fluoroisoindoline-1,3-dione (13.5 g, 48.8 mmol, 95.5% yield) was obtained. [M+H]⁺=277.

### Step 6: tert-butyl (1r,3r)-3-((2-(2,6-dioxopiperidin-3-yl)-1,3-dioxoisoindolin-5-yl)(methyl)amino)cyclobutane-1-carboxylate

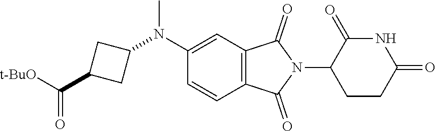

To a solution of tert-butyl (1r,3r)-3-(methylamino)cyclobutane-1-carboxylate (3.50 g, 18.9 mmol) in DMF (30.0 mL) was added 2-(2,6-dioxopiperidin-3-yl)-5-fluoroisoindoline-1,3-dione (2.61 g, 9.45 mmol) and DIPEA (4.88 g, 37.7 mmol, 6.58 mL) at 25° C. The mixture was stirred at 100° C. for 16 hrs. The reaction mixture was quenched by adding H₂O (50.0 mL) at 0° C., and then diluted with EA (100 mL×3) which is extracted with sat. aq. NaCl solution (100 mL×2). The combined organic layers were dried over Na₂SO₄, filtered and concentrated under reduced pressure to give a residue. The residue was purified by prep-HPLC (column: Phenomenex luna C18 250 mm*100 mm*10 um; mobile phase: [water (0.1% TFA)-ACN]; B %: 40%-70%, 25 min). tert-butyl (1r,3r)-3-((2-(2,6-dioxopiperidin-3-yl)-1,3-dioxoisoindolin-5-yl)(methyl)amino)cyclobutane-1-carboxylate (3.00 g, 6.80 mmol) was obtained [M+H]⁺=442.2.

### Step 7: (1r,3r)-3-((2-(2,6-dioxopiperidin-3-yl)-1,3-dioxoisoindolin-5-yl)(methyl)amino)cyclobutene-1-carboxylic acid

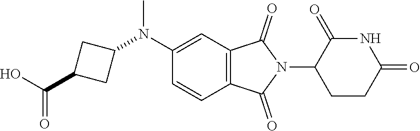

To a solution of (1r,3r)-tert-butyl 3-((2-(2,6-dioxopiperidin-3-yl)-1,3-dioxoisoindolin-5-yl)(methyl)amino)cyclobutanecarboxylate (2.80 g, 6.34 mmol) in HCl/dioxane (4.00 M, 28.0 mL) stirred at 25° C. for 1 hr. The reaction mixture was concentrated under reduced pressure to remove HCl/dioxane. And then the residual dioxane was removed by freeze-drying. (1r,3r)-3-((2-(2,6-dioxopiperidin-3-yl)-1,3-dioxoisoindolin-5-yl)(methyl)amino)cyclobutanecarboxylic acid (2.32 g) was obtained. [M+H]⁺=386.2

### Step 8: 5-(((1r,3r)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)cyclobutyl)(methyl)amino)-2-(2,6-dioxopiperidin-3-yl)isoindoline-1,3-dione

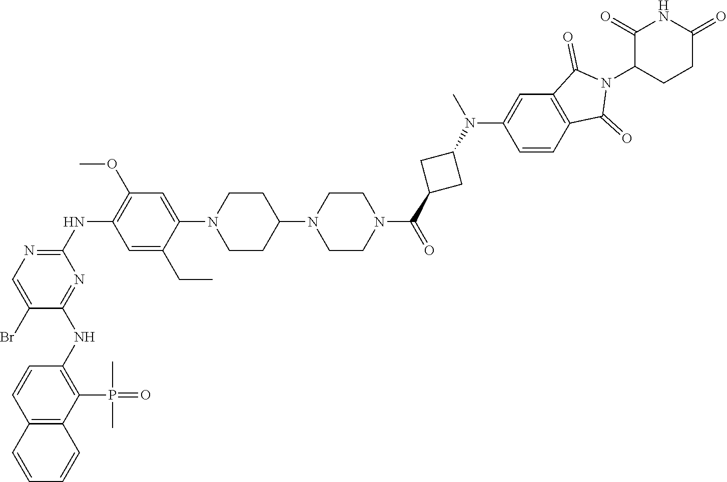 [SMILES: CCc1cc(Nc2ncc(Br)c(Nc3ccc4ccccc4c3P(C)(C)=O)n2)c(OC)cc1N1CCC(N2CCN(C(=O)[C@H]3C[C@H](N(C)c4ccc5c(c4)C(=O)N(C4CCC(=O)NC4=O)C5=O)C3)CC2)CC1]

The titled compound (12 mg, 26%) was prepared in a manner similar to that in Example 208 step 8 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and (1r,3r)-3-((2-(2,6-dioxopiperidin-3-yl)-1,3-dioxoisoindolin-5-yl)(methyl)amino)cyclobutene-1-carboxylic acid. 1H NMR (400 MHz, DMSO) δ 12.64 (s, 1H), 11.07 (s, 1H), 8.86 (dd, J=8.1, 1.4 Hz, 2H), 8.82 (s, 1H), 8.27 (s, 2H), 7.90 (d, J=9.9 Hz, 1H), 7.67 (d, J=8.5 Hz, 1H), 7.38 (s, 1H), 7.14-7.07 (m, 1H), 7.05-7.00 (m, 1H), 6.81 (d, J=2.4 Hz, 1H), 5.06 (dd, J=12.9, 5.4 Hz, 1H), 4.39-4.32 (m, 1H), 3.77 (d, J=1.0 Hz, 3H), 3.55-3.41 (m, 3H), 3.35 (s, 1H), 3.28 (d, J=14.8 Hz, 1H), 3.05-3.01 (m, 6H), 2.93-2.85 (m, 1H), 2.71 (t, J=10.4 Hz, 2H), 2.61-2.57 (m, 7H), 2.48-2.43 (m, 2H), 2.43-2.36 (m, 1H), 2.29 (d, J=9.6 Hz, 1H), 2.02 (d, J=14.4 Hz, 8H), 1.85 (s, 2H), 1.60 (d, J=11.6 Hz, 2H), 0.92 (t, J=6.8 Hz, 3H); [M+H]⁺=1061.6.

### Example 237: 3-(5-((R)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)pyrrolidin-1-yl)-1-oxoisoindolin-2-yl)piperidine-2,6-dione

### Step 1: methyl (S)-2-cyano-4-(3-(hydroxymethyl)pyrrolidin-1-yl)benzoate

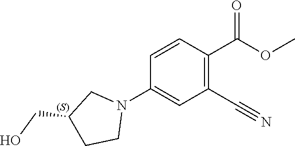

To a stirred mixture of methyl 2-cyano-4-fluorobenzoate (10.0 g, 55.819 mmol) and (S)-pyrrolidin-3-ylmethanol (6.8 g, 66.983 mmol) in DMSO (80 mL) was added DIEA (14.4 g, 111.638 mmol) and stirred overnight at 60° C. The resulting mixture was extracted with EtOAc (500 mL). The combined organic layers were washed with brine (100 mL), dried over anhydrous Na₂SO₄. After filtration, the filtrate was concentrated under reduced pressure. methyl (S)-2-cyano-4-(3-(hydroxymethyl)pyrrolidin-1-yl)benzoate (10.0 g, 98.3%) was obtained. [M+H]⁺=261.

### Step 2: methyl (S)-2-formyl-4-(3-(hydroxymethyl)pyrrolidin-1-yl)benzoate

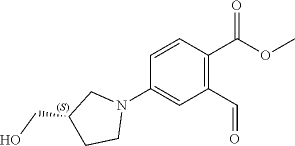

To a stirred mixture of methyl (S)-2-cyano-4-(3-(hydroxymethyl)pyrrolidin-1-yl)benzoate (10.0 g, 38.418 mmol) in AcOH (100 mL) and H₂O (50 mL) was added Raney-Ni (10.0 g, 116.720 mmol) in portions and stirred overnight at 40° C. under hydrogen atmosphere. The resulting mixture was filtered, the filter cake was washed with DCM and MeOH (300 mL). The filtrate was concentrated under reduced pressure. The filtrate was extracted with EtOAc (500 mL). The combined organic layers were washed with brine (500 mL), dried over anhydrous Na₂SO₄. After filtration, the filtrate was concentrated under reduced pressure. The residue was purified by silica gel column chromatography, eluted with CH₂Cl₂/MeOH (9:1) to afford product (5.0 g, 49.4%). [M+H]⁺=264.

### Step 3: 3-(5-((S)-3-(hydroxymethyl)pyrrolidin-1-yl)-1-oxoisoindolin-2-yl)piperidine-2,6-dione

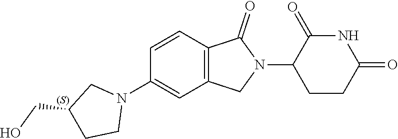

A mixture of 3-aminopiperidine-2,6-dione hydrochloride (4.7 g, 28.485 mmol) and DIEA (4.9 g, 37.980 mmol) in DMF (50 mL) was stirred for 5 h at room temperature. The mixture was acidified to pH<7 with AcOH (5.7 g, 94.950 mmol) followed by the addition of methyl (S)-2-formyl-4-(3-(hydroxymethyl)pyrrolidin-1-yl)benzoate (5.0 g, 18.990 mmol) in DCE (10.00 mL) and DMF (2 mL) at room temperature. The resulting mixture was stirred overnight at room temperature. To the above mixture was added NaBH₃CN (3.6 g, 56.970 mmol) in portions at room temperature. The resulting mixture was stirred for additional 2 h at room temperature. The reaction was quenched with water (50 mL) at room temperature. The resulting mixture was concentrated under reduced pressure. The residue was purified by silica gel column chromatography, eluted with CH₂Cl₂/MeOH (5:1) to afford crude product, which was purified by trituration with DCM (40 mL) to afford the product (2.1 g, 33.2%). [M+H]⁺=344.0.

### Step 3: ((3S)-1-(2-(2,6-dioxopiperidin-3-yl)-1-oxoisoindolin-5-yl)pyrrolidin-3-yl)methyl methanesulfonate

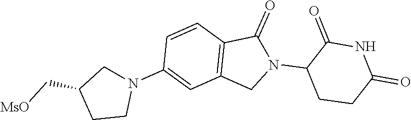 [SMILES: CS(=O)(=O)OC[C@H]1CCN(c2ccc3c(c2)CN(C2CCC(=O)NC2=O)C3=O)C1]

The titled compound (120 mg, 44%) was prepared in a manner similar to that in Example 207 step 4 from 3-(5-((S)-3-(hydroxymethyl)pyrrolidin-1-yl)-1-oxoisoindolin-2-yl)piperidine-2,6-dione and MsCl. [M+H]⁺=422.2.

### Step 4: 3-(5-((R)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)pyrrolidin-1-yl)-1-oxoisoindolin-2-yl)piperidine-2,6-dione

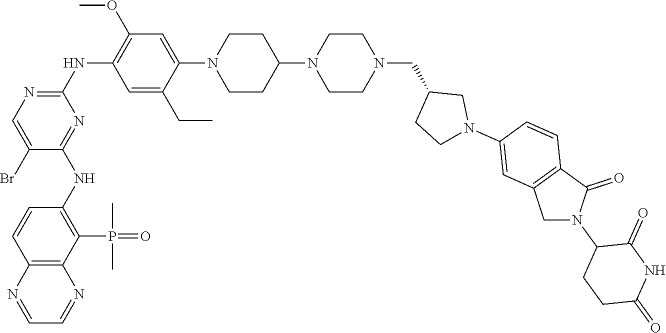 [SMILES: CCc1cc(Nc2ncc(Br)c(Nc3ccc4nccnc4c3P(C)(C)=O)n2)c(OC)cc1N1CCC(N2CCN(C[C@H]3CCN(c4ccc5c(c4)CN(C4CCC(=O)NC4=O)C5=O)C3)CC2)CC1]

The titled compound (11 mg, 24%) was prepared in a manner similar to that in Example 207 step 5 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and ((3S)-1-(2-(2,6-dioxopiperidin-3-yl)-1-oxoisoindolin-5-yl)pyrrolidin-3-yl)methyl methanesulfonate. 1H NMR (400 MHz, DMSO) δ 12.65 (s, 1H), 10.93 (s, 1H), 8.86 (dd, J=7.7, 1.9 Hz, 2H), 8.82 (s, 1H), 8.27 (s, 2H), 7.90 (d, J=9.0 Hz, 1H), 7.48 (d, J=8.3 Hz, 1H), 7.38 (s, 1H), 6.81 (s, 1H), 6.62 (d, J=9.9 Hz, 2H), 5.03 (dd, J=13.3, 5.1 Hz, 1H), 4.30 (dd, J=16.8, 3.6 Hz, 1H), 4.18 (dd, J=16.8, 2.8 Hz, 1H), 3.77 (s, 3H), 3.45 (t, J=8.0 Hz, 1H), 3.39 (s, 2H), 3.30-3.27 (m, 2H), 3.06-3.01 (m, 3H), 2.93-2.86 (m, 1H), 2.71 (t, J=10.9 Hz, 2H), 2.59-2.52 (m, 6H), 2.48 (s, 2H), 2.40-2.31 (m, 5H), 2.11 (d, J=6.6 Hz, 1H), 2.02 (d, J=14.4 Hz, 7H), 1.98-1.92 (m, 1H), 1.87 (d, J=10.4 Hz, 2H), 1.73 (dd, J=12.2, 7.8 Hz, 1H), 1.59 (dd, J=20.1, 11.4 Hz, 2H), 0.93 (t, J=7.2 Hz, 3H); [M+H]⁺=1019.6.

### Example 238: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2-chloro-6-fluorophenyl)piperidine-2,6-dione

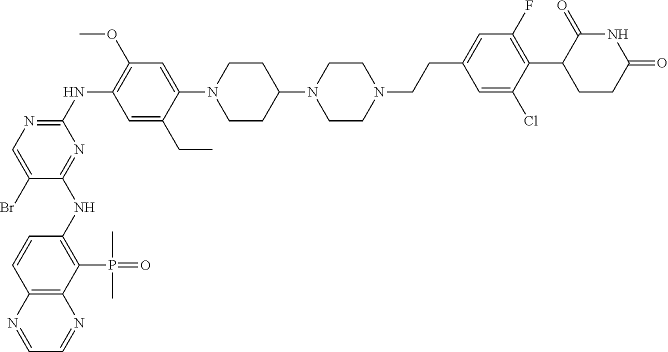 [SMILES: CCc1cc(Nc2ncc(Br)c(Nc3ccc4nccnc4c3P(C)(C)=O)n2)c(OC)cc1N1CCC(N2CCN(CCc3cc(F)c(C4CCC(=O)NC4=O)c(Cl)c3)CC2)CC1]

The titled compound was prepared in a manner similar to that in Example 207. 1H NMR (400 MHz, DMSO) δ 12.57 (s, 1H), 10.88 (s, 1H), 8.80 (dd, J=7.6, 1.8 Hz, 2H), 8.75 (s, 1H), 8.20 (s, 2H), 7.83 (d, J=9.5 Hz, 1H), 7.31 (s, 1H), 7.20 (s, 1H), 7.10 (d, J=11.1 Hz, 1H), 6.74 (s, 1H), 4.27 (dd, J=12.4, 5.4 Hz, 1H), 3.70 (s, 3H), 3.30 (s, 3H), 2.95 (d, J=11.1 Hz, 2H), 2.77-2.60 (m, 6H), 2.50-2.46 (m, 8H), 2.42-2.37 (m, 2H), 2.28 (d, J=17.7 Hz, 1H), 1.95 (d, J=14.4 Hz, 6H), 1.90 (s, 1H), 1.80 (d, J=10.7 Hz, 2H), 1.51 (dd, J=20.1, 11.2 Hz, 2H), 0.86 (t, J=7.0 Hz, 3H); [M+H]⁺=961.5.

### Example 239: 3-(6-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)-1-oxoisoindolin-2-yl)piperidine-2,6-dione

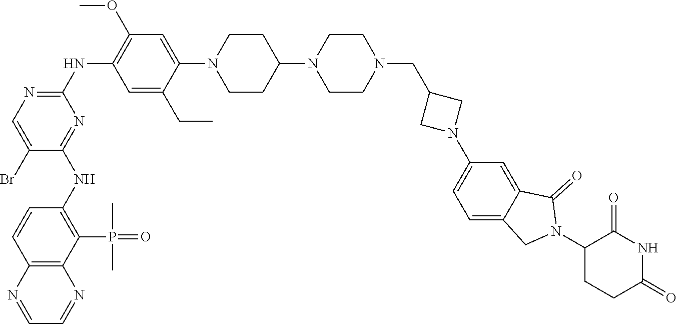 [SMILES: CCc1cc(Nc2ncc(Br)c(Nc3ccc4nccnc4c3P(C)(C)=O)n2)c(OC)cc1N1CCC(N2CCN(CC3CN(c4ccc5c(c4)C(=O)N(C4CCC(=O)NC4=O)C5)C3)CC2)CC1]

The titled compound was prepared in a manner similar to that in Example 237. 1H NMR (400 MHz, DMSO) δ 12.64 (s, 1H), 10.96 (s, 1H), 8.86 (dt, J=20.8, 10.4 Hz, 3H), 8.27 (s, 2H), 7.92 (s, 1H), 7.37 (d, J=7.7 Hz, 2H), 6.81 (s, 1H), 6.70-6.67 (m, 2H), 5.08 (dd, J=13.2, 5.1 Hz, 1H), 4.31 (d, J=16.6 Hz, 1H), 4.19 (d, J=16.5 Hz, 1H), 3.98 (t, J=7.5 Hz, 2H), 3.77 (s, 3H), 3.51-3.48 (m, 3H), 3.01 (d, J=10.5 Hz, 2H), 2.95-2.87 (m, 2H), 2.71 (t, J=11.2 Hz, 2H), 2.62 (d, J=17.2 Hz, 2H), 2.58 (d, J=7.2 Hz, 4H), 2.47 (d, J=7.5 Hz, 2H), 2.43-2.31 (m, 5H), 2.00 (dd, J=21.5, 9.8 Hz, 8H), 1.87 (d, J=11.1 Hz, 2H), 1.58 (d, J=8.9 Hz, 2H), 0.93 (t, J=7.1 Hz, 3H); [M+H]⁺=1005.6.

### Example 240: 3-(5-((S)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)pyrrolidin-1-yl)-1-oxoisoindolin-2-yl)piperidine-2,6-dione

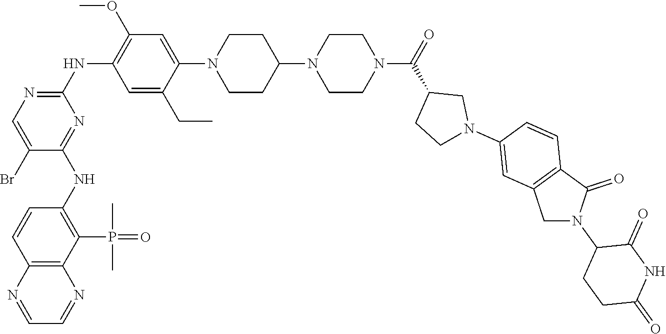 [SMILES: CCc1cc(Nc2ncc(Br)c(Nc3ccc4nccnc4c3P(C)(C)=O)n2)c(OC)cc1N1CCC(N2CCN(C(=O)[C@H]3CCN(c4ccc5c(c4)CN(C4CCC(=O)NC4=O)C5=O)C3)CC2)CC1]

The titled compound was prepared in a manner similar to that in Example 227. 1H NMR (400 MHz, DMSO) δ 12.65 (s, 1H), 10.93 (s, 1H), 8.86 (dt, J=17.8, 8.9 Hz, 3H), 8.28 (d, J=3.3 Hz, 2H), 7.90 (d, J=8.6 Hz, 1H), 7.50 (d, J=8.3 Hz, 1H), 7.38 (s, 1H), 6.82 (s, 1H), 6.65 (d, J=8.9 Hz, 2H), 5.04 (dd, J=13.3, 5.1 Hz, 1H), 4.31 (dd, J=16.8, 3.3 Hz, 1H), 4.19 (dd, J=17.0, 2.6 Hz, 1H), 3.77 (s, 3H), 3.61-3.54 (m, 4H), 3.51 (s, 1H), 3.45-3.37 (m, 4H), 3.04 (d, J=9.2 Hz, 2H), 2.93-2.87 (m, 1H), 2.73 (t, J=11.3 Hz, 2H), 2.60 (t, J=17.0 Hz, 4H), 2.48 (d, J=7.7 Hz, 2H), 2.42-2.31 (m, 2H), 2.22 (d, J=6.9 Hz, 1H), 2.13 (s, 1H), 2.02 (d, J=14.4 Hz, 7H), 1.98-1.93 (m, 1H), 1.88 (s, 2H), 1.63 (s, 2H), 0.93 (t, J=7.1 Hz, 3H); [M+H]⁺=1033.6.

### Example 241: 3-(5-((R)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)pyrrolidin-1-yl)-1-oxoisoindolin-2-yl)piperidine-2,6-dione

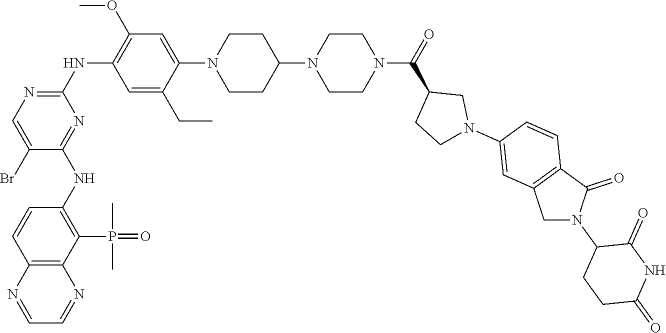 [SMILES: CCc1cc(Nc2ncc(Br)c(Nc3ccc4nccnc4c3P(C)(C)=O)n2)c(OC)cc1N1CCC(N2CCN(C(=O)[C@@H]3CCN(c4ccc5c(c4)CN(C4CCC(=O)NC4=O)C5=O)C3)CC2)CC1]

The titled compound was prepared in a manner similar to that in Example 227. 1H NMR (400 MHz, DMSO) δ 12.65 (s, 1H), 10.93 (s, 1H), 8.85 (t, J=11.9 Hz, 3H), 8.27 (s, 2H), 7.90 (d, J=9.2 Hz, 1H), 7.49 (d, J=8.2 Hz, 1H), 7.38 (s, 1H), 6.82 (s, 1H), 6.65 (d, J=9.1 Hz, 2H), 5.04 (dd, J=13.3, 5.0 Hz, 1H), 4.31 (dd, J=17.0, 3.5 Hz, 1H), 4.20 (d, J=17.0 Hz, 1H), 3.77 (s, 3H), 3.58-3.53 (m, 6H), 3.45 (s, 3H), 3.03 (d, J=9.6 Hz, 2H), 2.90 (t, J=12.8 Hz, 1H), 2.73 (t, J=11.0 Hz, 2H), 2.60 (t, J=17.7 Hz, 4H), 2.54 (s, 1H), 2.47 (s, 1H), 2.40 (s, 1H), 2.38-2.33 (m, 1H), 2.21 (s, 1H), 2.13 (s, 1H), 2.02 (d, J=14.4 Hz, 7H), 1.99-1.93 (m, 1H), 1.86 (s, 2H), 1.63 (s, 2H), 0.92 (t, J=7.6 Hz, 3H); [M+H]⁺=1033.7.

### Example 244: 3-(5-(4-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)piperidin-1-yl)-1-oxoisoindolin-2-yl)piperidine-2,6-dione

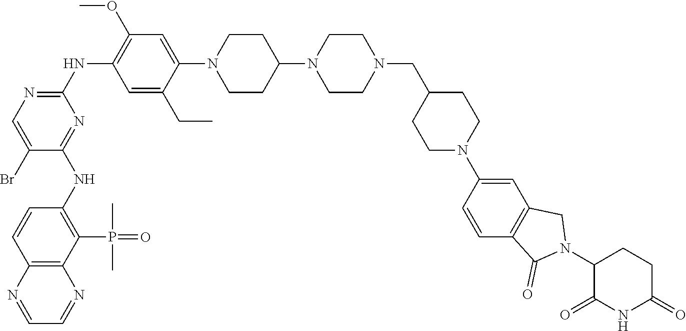 [SMILES: CCc1cc(Nc2ncc(Br)c(Nc3ccc4nccnc4c3P(C)(C)=O)n2)c(OC)cc1N1CCC(N2CCN(CC3CCN(c4ccc5c(c4)CN(C4CCC(=O)NC4=O)C5=O)CC3)CC2)CC1]

The titled compound was prepared in a manner similar to that in Example 237. 1H NMR (400 MHz, DMSO) δ 12.64 (s, 1H), 10.92 (s, 1H), 8.85 (dt, J=19.6, 9.8 Hz, 3H), 8.27 (s, 2H), 7.90 (d, J=9.2 Hz, 1H), 7.48 (d, J=9.0 Hz, 1H), 7.38 (s, 1H), 6.81 (s, 1H), 6.61 (d, J=7.5 Hz, 2H), 5.03 (dd, J=13.3, 5.2 Hz, 1H), 4.30 (d, J=16.4 Hz, 1H), 4.18 (d, J=16.5 Hz, 1H), 3.77 (s, 3H), 3.51 (t, J=7.4 Hz, 1H), 3.40-3.36 (m, 4H), 3.01 (d, J=10.5 Hz, 2H), 2.97-2.93 (m, 1H), 2.88 (d, J=12.3 Hz, 1H), 2.70 (t, J=11.1 Hz, 2H), 2.62-2.54 (m, 5H), 2.47 (d, J=7.5 Hz, 1H), 2.38-2.28 (m, 6H), 2.16 (s, 1H), 2.02 (d, J=14.4 Hz, 7H), 1.97-1.92 (m, 1H), 1.86 (d, J=10.6 Hz, 2H), 1.72-1.49 (m, 6H), 0.92 (t, J=7.1 Hz, 3H); [M+H]⁺=1033.7.

### Example 245: 3-(4-(((1r,3r)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)cyclobutyl)(methyl)amino)phenyl)piperidine-2,6-dione

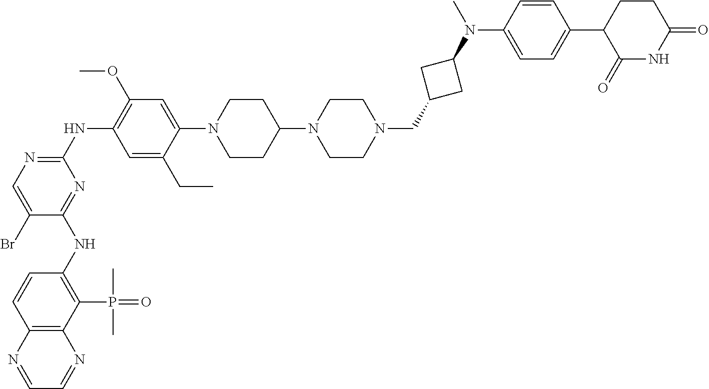 [SMILES: CCc1cc(Nc2ncc(Br)c(Nc3ccc4nccnc4c3P(C)(C)=O)n2)c(OC)cc1N1CCC(N2CCN(C[C@H]3C[C@H](N(C)c4ccc(C5CCC(=O)NC5=O)cc4)C3)CC2)CC1]

The titled compound was prepared in a manner similar to that in Example 207. 1H NMR (400 MHz, DMSO) δ 12.64 (s, 1H), 10.76 (s, 1H), 8.85 (dt, J=22.0, 11.0 Hz, 3H), 8.27 (s, 2H), 7.90 (d, J=9.0 Hz, 1H), 7.38 (s, 1H), 7.00 (d, J=8.6 Hz, 2H), 6.81 (s, 1H), 6.72 (t, J=6.9 Hz, 2H), 3.83-3.79 (m, 1H), 3.77 (s, 3H), 3.70 (dd, J=10.9, 4.9 Hz, 1H), 3.01 (d, J=10.7 Hz, 2H), 2.74 (s, 2H), 2.70 (t, J=11.2 Hz, 2H), 2.64-2.60 (m, 1H), 2.53 (s, 3H), 2.47-2.42 (m, 5H), 2.41-2.34 (m, 6H), 2.31 (s, 1H), 2.15-2.11 (m, 3H), 2.01 (dd, J=16.1, 9.5 Hz, 8H), 1.85 (d, J=10.4 Hz, 2H), 1.65 (d, J=10.3 Hz, 2H), 1.57 (d, J=11.5 Hz, 2H), 0.92 (t, J=7.1 Hz, 3H); [M+H]⁺=978.7.

### Example 246: 3-(5-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)-1-oxoisoindolin-2-yl)piperidine-2,6-dione

### Step 1: 1-(3-cyano-4-(methoxycarbonyl)phenyl)azetidine-3-carboxylic acid

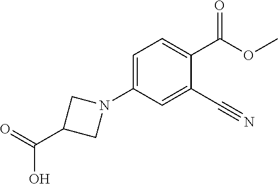 [SMILES: COC(=O)c1ccc(N2CC(C(=O)O)C2)cc1C#N]

To a solution of methyl 2-cyano-4-fluorobenzoate (5.00 g, 27.9 mmol) and azetidine-3-carboxylic acid (4.99 g, 36.3 mmol) in DMSO (35.0 mL). The DIPEA (9.02 g, 69.8 mmol) was added to the reaction mixture. The reaction mixture was warmed to 100° C. for 0.5 hr and then stirred at 100° C. for 3 hrs. The reaction mixture was diluted with ACN (35.0 mL) and stirred for 15 min, then filtered to get filtrate and adjusted the filtrate pH=8 by sat.aq.NaHCO₃ solution (40.0 mL). The mixture was extracted with EtOAc (50.0 mL). Water phase was adjusted pH=3 by 6N HCl (50.0 mL), extracted with EtOAc (50.0 mL×2). The combine organic phase was washed with brine (50.0 mL), dried over anhydrous Na₂SO₄, filtered and concentrated in vacuum to obtain product (6.00 g, crude). [M−H]−=259.1.

### Step 2: 1-(3-formvl-4-(methoxycarbonyl)phenyl)azetidine-3-carboxylic acid

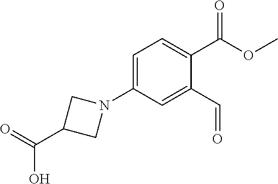 [SMILES: COC(=O)c1ccc(N2CC(C(=O)O)C2)cc1C=O]

To a solution of Raney-Ni (2.00 g, 11.7 mmol) in HCOOH (20.0 mL) at 25° C.,1-(3-cyano-4-(methoxycarbonyl)phenyl)azetidine-3-carboxylic acid in HCOOH (20.0 mL) was added to the reaction mixture at 25° C. Then the mixture was stirred at 25° C. for 15 min. The reaction mixture was diluted with solvent H₂O (20.0 mL) and extracted with EtOAc (40.0 mL×2). The combined organic phase was washed with brine (40.0 mL), dried over anhydrous Na₂SO₄, filtered and concentrated in vacuum to obtain product (5.50 g, crude). [M−H]⁻=262.1.

### Step 3: 1-(2-(2,6-dioxopiperidin-3-yl)-1-oxoisoindolin-5-yl)azetidine-3-carboxylic acid

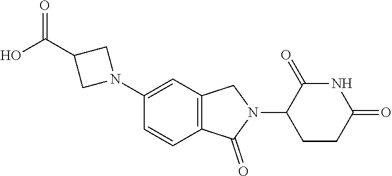 [SMILES: O=C1CCC(N2Cc3cc(N4CC(C(=O)O)C4)ccc3C2=O)C(=O)N1]

To a solution of 1-(3-formyl-4-(methoxycarbonyl)phenyl)azetidine-3-carboxylic acid (5.50 g, 20.9 mmol) and 3-aminopiperidine-2,6-dione hydrochloride (3.44 g, 20.9 mmol) in DMF (35.0 mL) was added DIPEA (8.10 g, 62.7 mmol) at 25° C. The mixture was stirred at 25° C. for 1 hr. Then CH₃COOH (4.39 g, 73.1 mmol) and NaBH₃CN (2.63 g, 41.8 mmol) was added to the reaction mixture at 25° C. The mixture was stirred at 25° C. for 1 hr. The mixture was adjust pH=8 by DIPEA. The mixture was stirred at 25° C. for 1 hr. LCMS showed the reaction was completed. The mixture was concentrated under reduced pressure to give a residue and purified by prep-HPLC ([water (0.225% FA)-ACN]; B %: 3%-35%, 20 min) to obtain the product (1.70 g, 4.95 mmol, 23.7% yield). [M+H]+=344.1.

### Step 4: 3-(5-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)-1-oxoisoindolin-2-yl)piperidine-2,6-dione

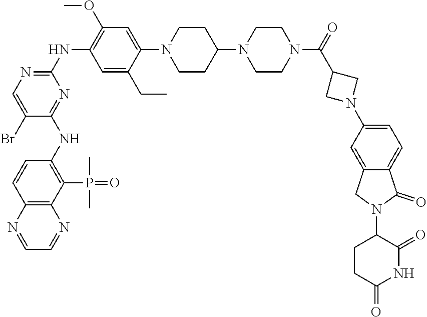 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc5c(c4)CN(C4CCC(=O)NC4=O)C5=O)C3)CC2)CC1]

The titled compound (11 mg, 26%) was prepared in a manner similar to that in Example 208 step 8 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 1-(2-(2,6-dioxopiperidin-3-yl)-1-oxoisoindolin-5-yl)azetidine-3-carboxylic acid. 1H NMR (400 MHz, DMSO) δ 12.58 (s, 1H), 10.87 (s, 1H), 8.79 (dt, J=18.9, 9.5 Hz, 3H), 8.21 (s, 2H), 7.83 (d, J=9.3 Hz, 1H), 7.44 (d, J=8.3 Hz, 1H), 7.31 (s, 1H), 6.75 (s, 1H), 6.50 (s, 1H), 6.45 (dd, J=8.3, 1.7 Hz, 1H), 4.97 (dd, J=13.3, 5.0 Hz, 1H), 4.25 (d, J=16.9 Hz, 1H), 4.12 (d, J=16.9 Hz, 1H), 4.06 (t, J=7.9 Hz, 2H), 3.94 (t, J=6.2 Hz, 2H), 3.85-3.79 (m, 1H), 3.71 (s, 3H), 3.44 (s, 2H), 2.96 (d, J=10.6 Hz, 2H), 2.87-2.79 (m, 1H), 2.65 (t, J=11.0 Hz, 2H), 2.51-2.46 (m, 5H), 2.41 (s, 2H), 2.38-2.25 (m, 3H), 1.98-1.87 (m, 8H), 1.79 (d, J=10.7 Hz, 2H), 1.54 (dd, J=20.3, 11.1 Hz, 2H), 0.86 (t, J=7.1 Hz, 3H); [M+H]⁺=1019.7.

### Example 247: 3-(4-(((1r,3r)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)cyclobutyl)(methyl)amino)phenyl)piperidine-2,6-dione

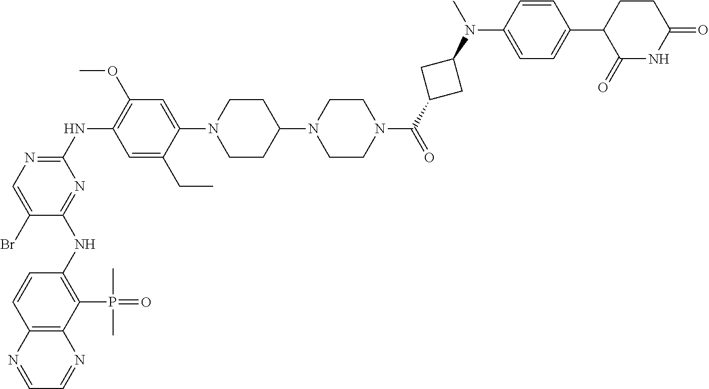 [SMILES: CCc1cc(Nc2ncc(Br)c(Nc3ccc4nccnc4c3P(C)(C)=O)n2)c(OC)cc1N1CCC(N2CCN(C(=O)[C@H]3C[C@H](N(C)c4ccc(C5CCC(=O)NC5=O)cc4)C3)CC2)CC1]

The titled compound was prepared in a manner similar to that in Example 208. 1H NMR (500 MHz, DMSO) δ 12.64 (s, 1H), 10.76 (s, 1H), 8.86 (d, J=6.9 Hz, 3H), 8.27 (s, 2H), 7.90 (d, J=9.3 Hz, 1H), 7.37 (s, 1H), 7.04-7.00 (m, 2H), 6.81 (s, 1H), 6.74 (d, J=8.6 Hz, 2H), 3.98-3.92 (m, 1H), 3.77 (s, 3H), 3.70 (dd, J=10.9, 5.0 Hz, 1H), 3.53-3.39 (m, 4H), 3.02 (d, J=9.6 Hz, 3H), 2.77 (d, J=15.9 Hz, 3H), 2.71 (s, 2H), 2.66-2.59 (m, 2H), 2.53 (d, J=8.6 Hz, 1H), 2.48-2.43 (m, 6H), 2.37 (d, J=9.7 Hz, 1H), 2.31 (d, J=9.2 Hz, 1H), 2.19-2.16 (m, 1H), 2.14-2.11 (m, 1H), 2.02 (d, J=14.4 Hz, 8H), 1.83 (s, 2H), 1.59 (d, J=11.5 Hz, 2H), 0.93 (t, J=7.2 Hz, 3H); [M+H]⁺=992.7.

### Example 248: 3-(4-((2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)amino)-1-oxoisoindolin-2-yl)piperidine-2,6-dione

### Step 1: 3-(4-amino-1-oxoisoindolin-2-yl)piperidine-2,6-dione

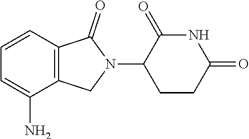 [SMILES: Nc1cccc2c1CN(C1CCC(=O)NC1=O)C2=O]

To a solution of 3-(4-nitro-1-oxoisoindolin-2-yl)piperidine-2,6-dione (1.50 g, 5.19 mmol) in MeOH (40 mL) was added Pd/C (wt % 100 mg) and stirred at 25° C. for 18 hours under H₂ atmosphere. The mixture was filtered and evaporated in vacuum to afford the product (0.90 g, 69%). [M+H]⁺=260.1.

### Step 2: 3-(4-((2-hydroxyethyl)amino)-1-oxoisoindolin-2-yl)piperidine-2,6-dione

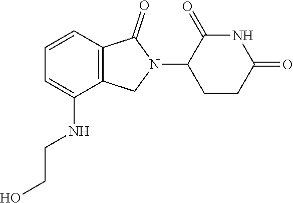 [SMILES: O=C1CCC(N2Cc3c(NCCO)cccc3C2=O)C(=O)N1]

A mixture of 3-(4-amino-1-oxoisoindolin-2-yl)piperidine-2,6-dione (0.65 g, 2.5 mmol), 2-bromoethan-1-ol (0.62 g, 5 mmol) and DIPEA (0.97 g, 7.5 mmol) in DMSO (15 mL) was stirred in a round bottom flask at 120° C. for 48 hours. The mixture was added brine (100 mL), extracted with DCM (100 mL×3). The organics were evaporated in vacuum to afford the crude product, which was further purified with silica gel column chromatography (DCM: MeOH=100: 1˜ 10:1 gradient elution) to give product (0.3 g, 40%). [M+H]⁺=304.1.

### Step 3: 2-((2-(2,6-dioxopiperidin-3-yl)-1-oxoisoindolin-4-yl)amino)ethyl methanesulfonate

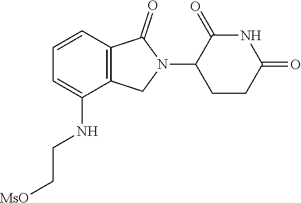 [SMILES: CS(=O)(=O)OCCNc1cccc2c1CN(C1CCC(=O)NC1=O)C2=O]

The titled compound (210 mg, 56%) was synthesized in a manner similar to that in Example 207 step 4 from 3-(4-((2-hydroxyethyl)amino)-1-oxoisoindolin-2-yl)piperidine-2,6-dione and MsCl.

### Step 4: 3-(4-((2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)amino)-1-oxoisoindolin-2-yl)piperidine-2,6-dione

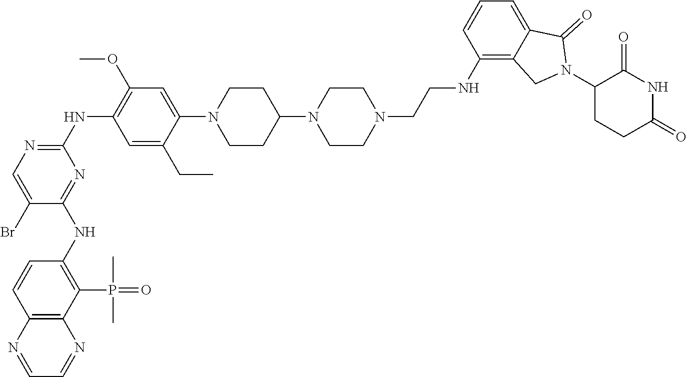 [SMILES: CCc1cc(Nc2ncc(Br)c(Nc3ccc4nccnc4c3P(C)(C)=O)n2)c(OC)cc1N1CCC(N2CCN(CCNc3cccc4c3CN(C3CCC(=O)NC3=O)C4=O)CC2)CC1]

The titled compound (10 mg, 36%) was synthesized in a manner similar to that in Example 207 step 5 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 2-((2-(2,6-dioxopiperidin-3-yl)-1-oxoisoindolin-4-yl)amino)ethyl methanesulfonate. 1H NMR (500 MHz, DMSO) δ 12.65 (s, 1H), 11.01 (s, 1H), 8.88-8.80 (m, 3H), 8.27 (s, 2H), 7.89 (s, 1H), 7.38 (s, 1H), 7.30 (t, J=7.7 Hz, 1H), 6.95 (d, J=7.2 Hz, 1H), 6.80 (d, J=8.2 Hz, 2H), 5.45 (s, 1H), 5.11 (dd, J=13.5, 5.4 Hz, 1H), 4.24 (d, J=17.3 Hz, 1H), 4.14 (d, J=16.8 Hz, 1H), 3.77 (s, 3H), 3.02 (d, J=10.5 Hz, 3H), 2.95-2.90 (m, 1H), 2.71 (t, J=10.7 Hz, 3H), 2.64 (s, 2H), 2.57-2.52 (m, 5H), 2.36-2.31 (m, J=20.9 Hz, 7H), 2.02 (d, J=14.3 Hz, 8H), 1.87-1.85 (m, 2H), 1.58 (d, J=12.0 Hz, 2H), 0.93 (t, J=7.2 Hz, 3H); [M+H]⁺=979.7.

### Example 249: 3-(6-((2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)(methyl)amino)-1-oxoisoindolin-2-yl)piperidine-2,6-dione

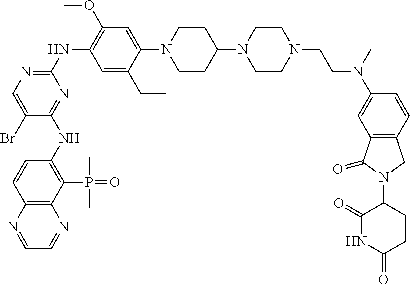 [SMILES: CCc1cc(Nc2ncc(Br)c(Nc3ccc4nccnc4c3P(C)(C)=O)n2)c(OC)cc1N1CCC(N2CCN(CCN(C)c3ccc4c(c3)C(=O)N(C3CCC(=O)NC3=O)C4)CC2)CC1]

The titled compound was prepared in a manner similar to that in Example 71. 1H NMR (400 MHz, DMSO) δ 12.64 (s, 1H), 10.96 (s, 1H), 8.85 (t, J=13.2 Hz, 3H), 8.27 (s, 2H), 7.90 (d, J=9.0 Hz, 1H), 7.38 (d, J=8.2 Hz, 2H), 7.00 (d, J=6.4 Hz, 1H), 6.92 (s, 1H), 6.81 (s, 1H), 5.09 (dd, J=13.4, 5.1 Hz, 1H), 4.31 (d, J=16.4 Hz, 1H), 4.18 (d, J=16.4 Hz, 1H), 3.77 (s, 3H), 3.54-3.50 (m, 3H), 3.01 (d, J=10.5 Hz, 2H), 2.97 (s, 3H), 2.94-2.89 (m, 1H), 2.70 (t, J=11.2 Hz, 2H), 2.64-2.57 (m, 2H), 2.55-2.52 (m, 3H), 2.48-2.41 (m, 6H), 2.38 (d, J=17.0 Hz, 1H), 2.31 (s, 1H), 2.02 (d, J=14.4 Hz, 8H), 1.86 (d, J=10.5 Hz, 2H), 1.57 (d, J=9.3 Hz, 2H), 0.92 (t, J=7.1 Hz, 3H); [M+H]⁺=993.5.

### Example 250: 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)azetidin-1-ylphenyl)piperidine-2,6-dione

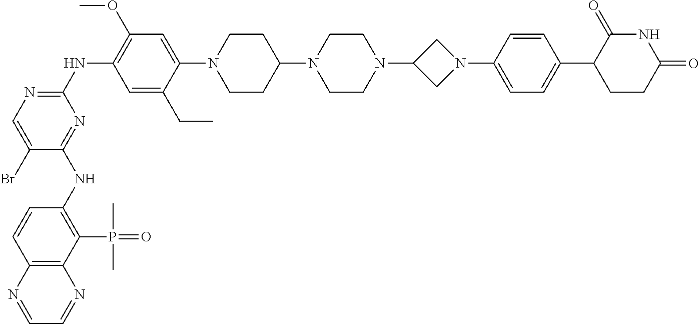 [SMILES: CCc1cc(Nc2ncc(Br)c(Nc3ccc4nccnc4c3P(C)(C)=O)n2)c(OC)cc1N1CCC(N2CCN(C3CN(c4ccc(C5CCC(=O)NC5=O)cc4)C3)CC2)CC1]

The titled compound was prepared in a manner similar to that in Example 200. 1H NMR (500 MHz, DMSO) δ 12.64 (s, 1H), 10.76 (s, 1H), 8.88-8.85 (m, 2H), 8.81 (d, J=7.3 Hz, 1H), 8.27 (s, 1H), 7.90 (d, J=9.8 Hz, 1H), 7.37 (s, 1H), 7.01 (d, J=8.4 Hz, 2H), 6.81 (s, 1H), 6.40 (d, J=8.4 Hz, 2H), 3.90 (t, J=7.0 Hz, 2H), 3.77 (s, 3H), 3.70 (dd, J=11.0, 4.9 Hz, 1H), 3.56 (t, J=6.2 Hz, 2H), 3.30 (s, 2H), 3.27-3.20 (m, 2H), 3.01 (d, J=11.0 Hz, 2H), 2.70 (t, J=11.0 Hz, 2H), 2.66-2.53 (m, 4H), 2.48-2.42 (m, 5H), 2.40-2.29 (m, 2H), 2.10 (dt, J=11.9, 3.9 Hz, 1H), 2.03 (s, 3H), 2.02-1.97 (m, 4H), 1.86 (dd, J=12.8, 1.7 Hz, 2H), 1.59 (dt, J=21.1, 7.1 Hz, 2H), 0.92 (t, J=6.6 Hz, 3H); [M+H]⁺=937.6.

### Example 251: 5-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)-2-(2,6-dioxopiperidin-3-yl)benzonitrile

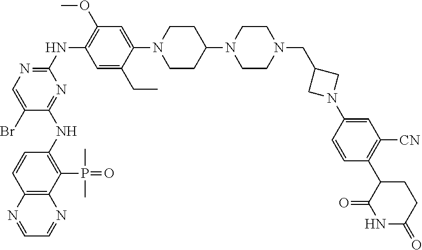 [SMILES: CCc1cc(Nc2ncc(Br)c(Nc3ccc4nccnc4c3P(C)(C)=O)n2)c(OC)cc1N1CCC(N2CCN(CC3CN(c4ccc(C5CCC(=O)NC5=O)c(C#N)c4)C3)CC2)CC1]

The titled compound was prepared in a manner similar to that in Example 200. ¹H NMR (500 MHz, DMSO) δ 12.65 (s, 1H), 10.91 (s, 1H), 8.87 (d, J=5.9 Hz, 2H), 8.82 (s, 1H), 8.28 (s, 1H), 8.27 (s, 1H), 7.90 (d, J=8.9 Hz, 1H), 7.38 (s, 1H), 7.22 (d, J=8.6 Hz, 1H), 6.81 (s, 1H), 6.80 (d, J=2.3 Hz, 1H), 6.70 (dd, J=8.5, 2.3 Hz, 1H), 4.01-3.95 (m, 3H), 3.77 (s, 3H), 3.50 (t, J=6.5 Hz, 2H), 3.01 (d, J=10.6 Hz, 2H), 2.94 (dt, J=14.0, 7.2 Hz, 1H), 2.84-2.76 (m, 1H), 2.71 (t, J=11.3 Hz, 2H), 2.60-2.51 (m, 8H), 2.47-2.23 (m, 7H), 2.06-1.97 (m, 7H), 1.86 (d, J=11.2 Hz, 2H), 1.58 (dt, J=20.9, 10.4 Hz, 2H), 0.93 (t, J=7.0 Hz, 3H); [M+H]⁺=975.6.

### Example 252: 3-(4-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)-2-fluorophenyl)piperidine-2,6-dione

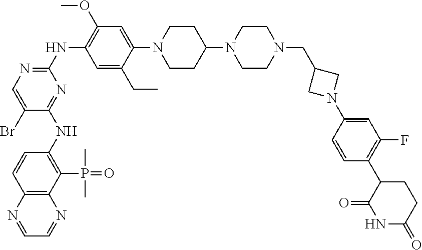 [SMILES: CCc1cc(Nc2ncc(Br)c(Nc3ccc4nccnc4c3P(C)(C)=O)n2)c(OC)cc1N1CCC(N2CCN(CC3CN(c4ccc(C5CCC(=O)NC5=O)c(F)c4)C3)CC2)CC1]

The titled compound was prepared in a manner similar to that in Example 200. ¹H NMR (500 MHz, DMSO) δ 12.64 (s, 1H), 10.78 (s, 1H), 8.85 (t, J=13.7 Hz, 3H), 8.27 (s, 2H), 7.90 (d, J=9.2 Hz, 1H), 7.38 (s, 1H), 7.03 (t, J=8.5 Hz, 1H), 6.81 (s, 1H), 6.26-6.12 (m, 2H), 3.92 (t, J=7.4 Hz, 2H), 3.85 (dd, J=12.2, 4.8 Hz, 1H), 3.77 (s, 3H), 3.45 (t, J=6.4 Hz, 2H), 3.01 (d, J=10.7 Hz, 2H), 2.96-2.87 (m, 1H), 2.71 (t, J=12.1 Hz, 3H), 2.61-2.52 (m, 9H), 2.43 (dd, J=41.1, 16.0 Hz, 4H), 2.32 (s, 1H), 2.12 (dt, J=14.3, 11.5 Hz, 1H), 2.04 (s, 3H), 2.01 (s, 3H), 1.94 (dd, J=9.2, 4.2 Hz, 1H), 1.86 (d, J=12.6 Hz, 2H), 1.57 (dd, J=20.1, 11.4 Hz, 2H), 0.93 (t, J=7.1 Hz, 3H); [M+H]⁺=968.8.

### Example 253: 3-(4-(3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)azetidin-1-yl)-2,6-difluorophenyl)piperidine-2,6-dione

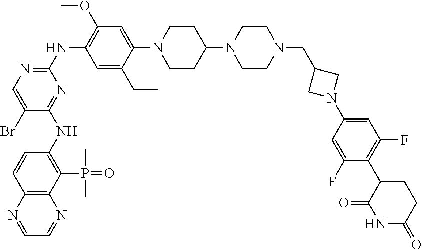 [SMILES: CCc1cc(Nc2ncc(Br)c(Nc3ccc4nccnc4c3P(C)(C)=O)n2)c(OC)cc1N1CCC(N2CCN(CC3CN(c4cc(F)c(C5CCC(=O)NC5=O)c(F)c4)C3)CC2)CC1]

The titled compound was prepared in a manner similar to that in Example 200. ¹H NMR (500 MHz, DMSO) δ 12.65 (s, 1H), 10.85 (s, 1H), 8.89-8.84 (m, 2H), 8.82 (s, 1H), 8.27 (s, 2H), 7.90 (d, J=9.0 Hz, 1H), 7.38 (s, 1H), 6.81 (s, 1H), 6.11 (d, J=11.2 Hz, 2H), 4.02 (dd, J=12.7, 5.1 Hz, 1H), 3.93 (t, J=7.8 Hz, 2H), 3.77 (s, 3H), 3.48 (t, J=6.6 Hz, 2H), 3.29 (s, 6H), 3.01 (d, J=9.8 Hz, 2H), 2.95-2.89 (m, 1H), 2.78 (dd, J=10.6, 5.3 Hz, 1H), 2.71 (t, J=11.6 Hz, 2H), 2.60-2.53 (m, 4H), 2.43-2.40 (m, 2H), 2.07 (dd, J=10.8, 5.8 Hz, 2H), 2.02 (d, J=14.3 Hz, 6H), 1.96-1.91 (m, 2H), 1.90-1.84 (m, 2H), 1.64-1.52 (m, 2H), 0.92 (t, J=6.8 Hz, 3H); [M+H]⁺=986.5.

### Example 254: 3-(4-(4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)piperidin-1-yl)-2,6-difluorophenyl)piperidine-2,6-dione

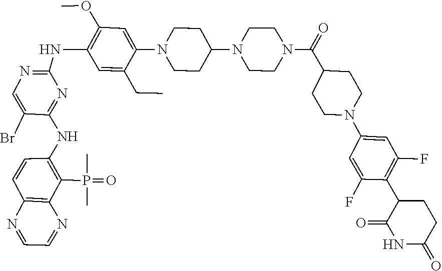 [SMILES: CCc1cc(Nc2ncc(Br)c(Nc3ccc4nccnc4c3P(C)(C)=O)n2)c(OC)cc1N1CCC(N2CCN(C(=O)C3CCN(c4cc(F)c(C5CCC(=O)NC5=O)c(F)c4)CC3)CC2)CC1]

The titled compound was prepared in a manner similar to that in Example 208. ¹H NMR (500 MHz, DMSO) δ 12.65 (s, 1H), 10.87 (s, 1H), 8.85 (dt, J=24.0, 12.0 Hz, 3H), 8.27 (s, 2H), 7.90 (d, J=9.2 Hz, 1H), 7.38 (s, 1H), 6.81 (s, 1H), 6.63 (d, J=12.8 Hz, 2H), 4.05 (dd, J=12.7, 4.9 Hz, 1H), 3.79 (d, J=14.6 Hz, 5H), 3.51 (d, J=38.9 Hz, 4H), 3.32 (s, 1H), 3.02 (d, J=11.1 Hz, 2H), 2.90-2.68 (m, 6H), 2.60-2.51 (m, 3H), 2.38 (dd, J=20.7, 6.6 Hz, 4H), 2.15-1.93 (m, 8H), 1.86 (d, J=10.7 Hz, 2H), 1.63 (dd, J=31.1, 11.9 Hz, 6H), 0.93 (t, J=7.1 Hz, 3H); [M+H]⁺=1030.7.

### Example 255: 3-(4-((R)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

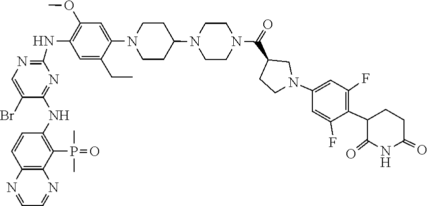 [SMILES: CCc1cc(Nc2ncc(Br)c(Nc3ccc4nccnc4c3P(C)(C)=O)n2)c(OC)cc1N1CCC(N2CCN(C(=O)[C@@H]3CCN(c4cc(F)c(C5CCC(=O)NC5=O)c(F)c4)C3)CC2)CC1]

The titled compound was prepared in a manner similar to that in Example 208. ¹H NMR (500 MHz, DMSO) δ 12.65 (s, 1H), 10.84 (s, 1H), 8.86 (dd, J=8.2, 1.7 Hz, 2H), 8.82 (s, 1H), 8.27 (s, 2H), 7.90 (d, J=9.9 Hz, 1H), 7.38 (s, 1H), 6.81 (s, 1H), 6.23 (d, J=12.2 Hz, 2H), 4.02 (dd, J=12.5, 4.9 Hz, 1H), 3.77 (s, 3H), 3.62-3.42 (m, 6H), 3.26 (dd, J=19.8, 11.2 Hz, 3H), 3.03 (d, J=10.4 Hz, 2H), 2.76 (dt, J=22.5, 8.3 Hz, 3H), 2.56 (d, J=22.9 Hz, 4H), 2.39 (d, J=23.6 Hz, 4H), 2.17 (dd, J=12.4, 5.4 Hz, 1H), 2.09 (dd, J=17.7, 9.0 Hz, 2H), 2.02 (d, J=14.4 Hz, 6H), 1.97-1.91 (m, 1H), 1.87 (d, J=10.4 Hz, 2H), 1.67-1.56 (m, 2H), 0.93 (t, J=7.1 Hz, 3H); [M+H]⁺=1014.6.

### Example 256: 3-(4-((2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)(methyl)amino)-1-oxoisoindolin-2-yl)piperidine-2,6-dione

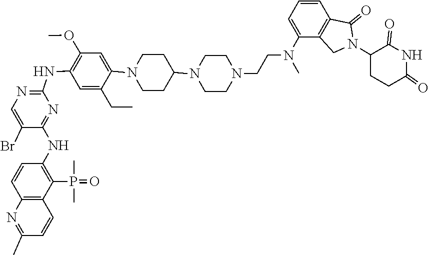 [SMILES: CCc1cc(Nc2ncc(Br)c(Nc3ccc4nc(C)ccc4c3P(C)(C)=O)n2)c(OC)cc1N1CCC(N2CCN(CCN(C)c3cccc4c3CN(C3CCC(=O)NC3=O)C4=O)CC2)CC1]

The titled compound was prepared in a manner similar to that in Example 71. ¹H NMR (500 MHz, DMSO) δ 11.76 (s, 1H), 10.99 (s, 1H), 8.56 (d, J=8.9 Hz, 1H), 8.29 (s, 1H), 8.22 (d, J=9.5 Hz, 2H), 7.97 (s, 1H), 7.87 (d, J=9.3 Hz, 1H), 7.45-7.33 (m, 3H), 7.15 (d, J=7.3 Hz, 1H), 7.00 (d, J=8.1 Hz, 1H), 6.73 (s, 1H), 5.12 (dd, J=13.2, 5.1 Hz, 1H), 4.51 (d, J=16.8 Hz, 1H), 4.38 (d, J=16.8 Hz, 1H), 3.75 (s, 3H), 2.97-2.87 (m, 6H), 2.68-2.57 (m, 8H), 2.47-2.23 (m, 12H), 2.04-1.93 (m, 8H), 1.83-1.76 (m, 2H), 1.51-1.40 (m, 2H), 0.76-0.65 (m, 3H); [M+H]⁺=1006.5.

### Example 257: 3-(5-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-2H-indazol-2-yl)piperidine-2,6-dione

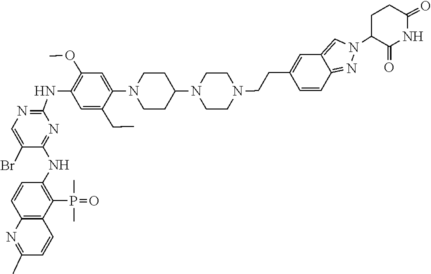 [SMILES: CCc1cc(Nc2ncc(Br)c(Nc3ccc4nc(C)ccc4c3P(C)(C)=O)n2)c(OC)cc1N1CCC(N2CCN(CCc3ccc4nn(C5CCC(=O)NC5=O)cc4c3)CC2)CC1]

The titled compound was prepared in a manner similar to that in Example 309. ¹H NMR (500 MHz, DMSO) δ 11.77 (s, 1H), 11.15 (s, 1H), 8.56 (d, J=9.0 Hz, 1H), 8.32 (d, J=21.4 Hz, 2H), 8.21 (s, 1H), 7.98 (s, 1H), 7.87 (d, J=9.0 Hz, 1H), 7.52 (d, J=8.6 Hz, 2H), 7.42 (d, J=8.9 Hz, 1H), 7.37 (s, 1H), 7.16 (d, J=9.1 Hz, 1H), 6.74 (s, 1H), 5.68 (dd, J=11.3, 5.2 Hz, 1H), 3.75 (s, 3H), 2.98-2.90 (m, 2H), 2.88-2.68 (m, 6H), 2.69-2.52 (m, 13H), 2.39-2.24 (m, 5H), 1.98 (d, J=13.3 Hz, 6H), 1.87-1.80 (m, 2H), 1.55-1.57 (m, 2H), 0.86-0.69 (m, 3H); [M+H]⁺=962.5

### Example 259: 5-(3-((3-(4-(4-((5-bromo-4-((5-(dimethylphosphoryl)quinoxalin-6-yl)amino)pyrimidin-2-yl)amino)-2-fluorophenyl)piperazin-1-yl)azetidin-1-yl)methyl)azetidin-1-yl)-2-(2,6-dioxopiperidin-3-yl)isoindoline-1,3-dione

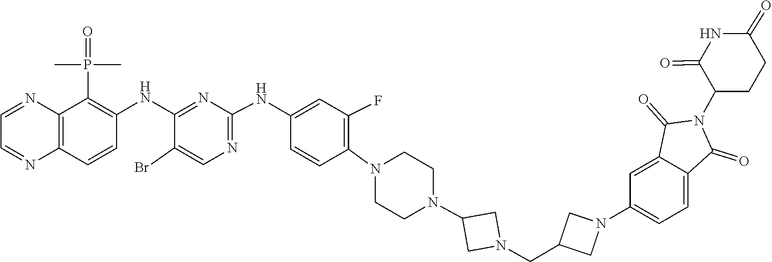 [SMILES: CP(C)(=O)c1c(Nc2nc(Nc3ccc(N4CCN(C5CN(CC6CN(c7ccc8c(c7)C(=O)N(C7CCC(=O)NC7=O)C8=O)C6)C5)CC4)c(F)c3)ncc2Br)ccc2nccnc12]

The titled compound was prepared in a manner similar to that in Example 59. ¹H NMR (400 MHz, dmso) δ 12.71 (s, 1H), 11.08 (s, 1H), 9.58 (s, 1H), 9.05-8.80 (m, J=12.1 Hz, 3H), 8.37 (s, 1H), 8.13 (d, J=9.4 Hz, 1H), 7.64 (d, J=17.0 Hz, 2H), 7.34-7.18 (m, 1H), 7.05-6.89 (m, 1H), 6.78 (s, 1H), 6.64 (d, J=6.7 Hz, 1H), 5.15-5.01 (m, J=7.6 Hz, 1H), 4.18-4.08 (m, 2H), 3.87-3.71 (m, 2H), 3.06-2.82 (m, 8H), 2.69-2.52 (m, J=28.7 Hz, 4H), 2.47-2.28 (m, 4H), 2.12-1.94 (m, J=14.4 Hz, 8H), 1.29-1.14 (m, 2H). [M+H]⁺=951.8.

### Example 260: 5-(3-(3-(4-(4-((5-bromo-4-((5-(dimethylphosphoryl)quinoxalin-6-yl)amino)pyrimidin-2-yl)amino)-2-fluorophenyl)piperazin-1-yl)azetidine-1-carbonyl)azetidin-1-yl)-2-(2,6-dioxopiperidin-3-yl)isoindoline-1,3-dione

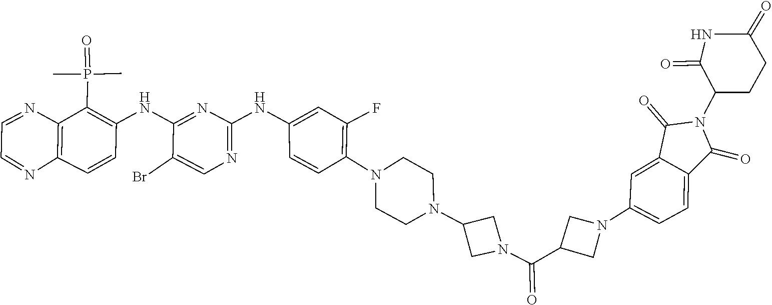 [SMILES: CP(C)(=O)c1c(Nc2nc(Nc3ccc(N4CCN(C5CN(C(=O)C6CN(c7ccc8c(c7)C(=O)N(C7CCC(=O)NC7=O)C8=O)C6)C5)CC4)c(F)c3)ncc2Br)ccc2nccnc12]

The titled compound was prepared in a manner similar to that in Example 57. ¹H NMR (400 MHz, dmso) δ 12.71 (s, 1H), 11.08 (s, 1H), 9.57 (s, 1H), 9.00-8.83 (m, 3H), 8.37 (s, 1H), 8.14 (d, J=9.6 Hz, 1H), 7.70-7.59 (m, 2H), 7.25 (d, J=8.0 Hz, 1H), 6.96 (t, J=9.2 Hz, 1H), 6.85-6.80 (m, 1H), 6.68 (d, J=8.4 Hz, 1H), 5.46-5.27 (m, 1H), 5.06 (dd, J=12.8, 5.4 Hz, 1H), 4.22-4.10 (m, 3H), 4.08-3.98 (m, 3H), 3.97-3.91 (m, 1H), 3.80-3.74 (m, 1H), 3.70-3.63 (m, 1H), 3.27-3.18 (m, 2H), 3.02-2.82 (m, 6H), 2.64-2.54 (m, 2H), 2.07-1.99 (m, 7H). [M+H]⁺=965.7.

### Example 264: 3-(3-(4-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)piperidin-1-yl)phenyl)piperidine-2,6-dione

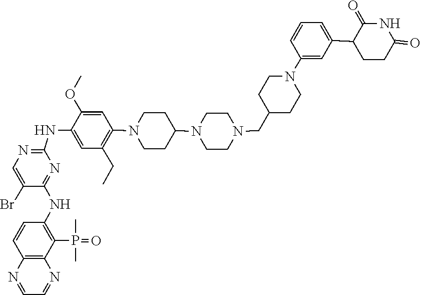 [SMILES: CCc1cc(Nc2ncc(Br)c(Nc3ccc4nccnc4c3P(C)(C)=O)n2)c(OC)cc1N1CCC(N2CCN(CC3CCN(c4cccc(C5CCC(=O)NC5=O)c4)CC3)CC2)CC1]

The titled compound was prepared in a manner similar to that in Example 207. ¹H NMR (500 MHz, DMSO) δ 12.65 (s, 1H), 10.79 (s, 1H), 8.94-8.74 (m, 3H), 8.34-8.22 (m, 2H), 8.17 (s, 1H), 7.96-7.83 (m, 1H), 7.37 (s, 1H), 7.09 (t, J=8.0 Hz, 1H), 6.81 (s, 1H), 6.41 (d, J=7.5 Hz, 2H), 6.35 (s, 1H), 3.78-3.71 (m, 4H), 3.44-3.36 (m, J=8.0 Hz, 2H), 3.29-3.15 (m, 4H), 3.06-2.97 (m, 2H), 2.87-2.80 (m, 1H), 2.74-2.67 (m, 2H), 2.64-2.53 (m, 4H), 2.47-2.41 (m, 4H), 2.38-2.21 (m, 6H), 2.20-2.08 (m, 2H), 2.05-1.98 (m, 5H), 1.89-1.82 (m, 2H), 1.64-1.53 (m, 5H), 0.98-0.87 (m, 3H). [M+H]⁺=978.6.

### Example 266: 3-(4-(4-(2-(3-(1-(4-((5-bromo-4-((5-(dimethylphosphoryl) quinoxaline-6-yl)amino)pyrimidin-2-yl)amino)-5-methoxy-2-methylphenyl)piperidin-4-yl)-3,6-diazabicyclo[3.1.1]heptan-6-yl)ethyl)piperidin-1-yl)-2,6-difluorophenyl)piperidine-2,6-dione

### Step 1: 8-(5-methoxy-2-methyl-4-nitrophenyl)-1,4-dioxa-8-azaspiro[4.5]decane

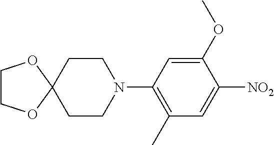 [SMILES: COc1cc(N2CCC3(CC2)OCCO3)c(C)cc1[N+](=O)[O-]]

To a stirred solution of 1-fluoro-5-methoxy-2-methyl-4-nitrobenzene (1.0 g, 5.4 mmol) and K₂CO₃ (1.49 g, 10.8 mmol) in DMSO (20 mL) was added 1,4-dioxa-8-azaspiro[4.5]decane (1.16 g, 8.1 mmol). The resulting mixture was stirred at 100° C. for 16 hour. The reaction was extracted with DCM (2×50.0 mL). The combined organic layer was washed with brine (2×50.0 mL), dried over Na₂SO₄ and concentrated under vacuum to afford the crude residue, which was purified with silica gel column chromatography (PE: EA=2:1˜ 0:100 gradient elution) to give the title product (1.33 g, 80%). [M+H]⁺=309.2.

### Step 2: 1-(5-methoxy-2-methyl-4-nitrophenyl)piperidin-4-one

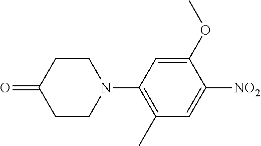 [SMILES: COc1cc(N2CCC(=O)CC2)c(C)cc1[N+](=O)[O-]]

To a stirred solution of 8-(5-methoxy-2-methyl-4-nitrophenyl)-1,4-dioxa-8-azaspiro[4.5]decane (1.33 g, 4.3 mmol) in THF (20 mL) was added HCl (4 mol/L). The resulting mixture was stirred at 60° C. for 4 hour.

The reaction was extracted with DCM (2×50.0 mL). The combined organic layer was washed with brine (2×50.0 mL), dried over Na2SO4 and concentrated under vacuum to afford the crude residue, which was purified with silica gel column chromatography (DCM:MeOH=10:1) to give the title product (800 mg, 70%). [M+H]⁺=265.1.

### Step 3: tert-butyl 3-(1-(5-methoxy-2-methyl-4-nitrophenyl)piperidin-4-yl)-3,6-diazabicyclo[3.1.1]heptane-6-carboxylate

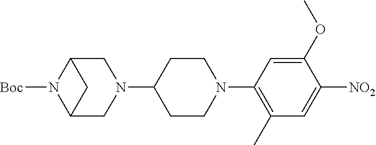 [SMILES: COc1cc(N2CCC(N3CC4CC(C3)N4C(=O)OC(C)(C)C)CC2)c(C)cc1[N+](=O)[O-]]

To a stirred solution of 1-(5-methoxy-2-methyl-4-nitrophenyl)piperidin-4-one (200 mg, 0.76 mmol) and tert-butyl 3,6-diazabicyclo[3.1.1]heptane-6-carboxylate (225 mg, 1.14 mmol) in DCE (10 mL) was added Ti(OiPr)₄ (215 mg, 0.76 mmol). The resulting mixture was stirred at rt for 2 hour. Then Na(OAc)₃BH (322 mg, 1.52 mmol) was added. The resulting mixture was stirred at rt for 16 hour. The reaction was extracted with DCM (2×50.0 mL). The combined organic layer was washed with brine (2×50.0 mL), dried over Na₂SO₄ and concentrated under vacuum to afford the crude residue, which was purified with silica gel column chromatography (DCM:MeOH=10:1) to give the title product (300 mg, 88%). [M+H]⁺=447.2.

### Step 4: tert-butyl 3-(1-(4-amino-5-methoxy-2-methylphenyl)piperidin-4-yl)-3,6-diazabicyclo[3.1.1]heptane-6-carboxylate

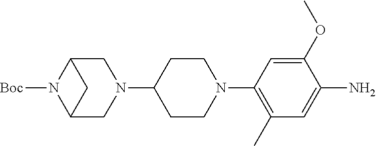 [SMILES: COc1cc(N2CCC(N3CC4CC(C3)N4C(=O)OC(C)(C)C)CC2)c(C)cc1N]

To a stirred solution of tert-butyl 3-(1-(5-methoxy-2-methyl-4-nitrophenyl)piperidin-4-yl)-3,6-diazabicyclo[3.1.1]heptane-6-carboxylate (300 mg, 0.66 mmol) in MeOH (10 mL) was added Pd/C (60 mg) under H₂. The resulting mixture was stirred at rt for 2 hour. The mixture was concentrated under vacuum to afford the title product (220 mg, 80%). [M+H]⁺=417.2.

### Step 5: (6-((2-((4-(4-(3,6-diazabicyclo[3.1.1]heptan-3-yl)piperidin-1-yl)-2-methoxy-5-methylphenyl)amino)-5-bromopyrimidin-4-yl)amino)quinoxalin-5-yl)dimethylphosphine oxide

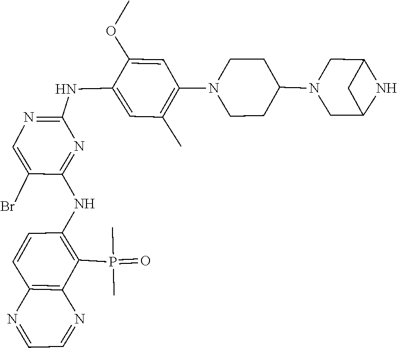 [SMILES: COc1cc(N2CCC(N3CC4CC(C3)N4)CC2)c(C)cc1Nc1ncc(Br)c(Nc2ccc3nccnc3c2P(C)(C)=O)n1]

To a stirred solution of (6-((5-bromo-2-chloropyrimidin-4-yl)amino)quinoxalin-5-yl)dimethylphosphine oxide (100 mg, 0.24 mmol) and tert-butyl 3-(1-(4-amino-5-methoxy-2-methylphenyl)piperidin-4-yl)-3,6-diazabicyclo[3.1.1]heptane-6-carboxylate (120 mg, 0.29 mmol) in t-BuOH (10 mL) was added MsOH (96 mg, 0.96 mmol). The resulting mixture was stirred at 95° C. for 16 hour. The reaction was extracted with DCM (2×50.0 mL). The combined organic layer was washed with brine (2×50.0 mL), dried over Na₂SO₄ and concentrated under vacuum to afford the crude residue, which was purified with silica gel column chromatography (DCM:MeOH=8:1) to give the title product (100 mg, 60%). [M+H]⁺=692.2.

### Step 6: 3-(4-(4-(2-(3-(1-(4-((5-bromo-4-((5-(dimethylphosphoryl)quinoxalin-6-yl)amino)pyrimidin-2-yl)amino)-5-methoxy-2-methylphenyl)piperidin-4-yl)-3,6-diazabicyclo[3.1.1]heptan-6-yl)ethyl)piperidin-1-yl)-2,6-difluorophenyl)piperidine-2,6-dione

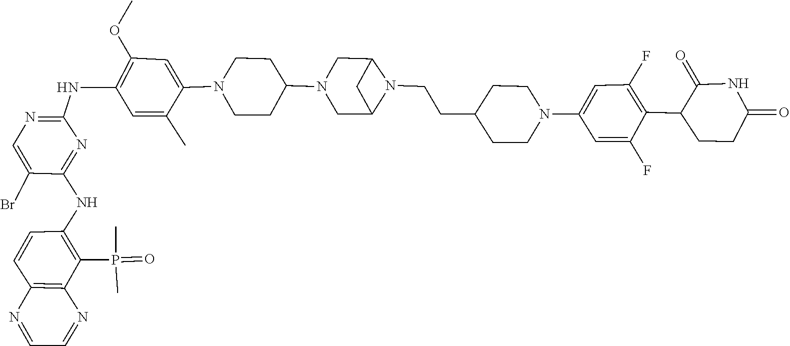 [SMILES: COc1cc(N2CCC(N3CC4CC(C3)N4CCC3CCN(c4cc(F)c(C5CCC(=O)NC5=O)c(F)c4)CC3)CC2)c(C)cc1Nc1ncc(Br)c(Nc2ccc3nccnc3c2P(C)(C)=O)n1]

To a stirred solution of (6-((2-((4-(4-(3,6-diazabicyclo[3.1.1]heptan-3-yl)piperidin-1-yl)-2-methoxy-5-methylphenyl)amino)-5-bromopyrimidin-4-yl)amino)quinoxalin-5-yl)dimethylphosphine oxide(30 mg, 0.043 mmol) and 2-(1-(4-(2,6-dioxopiperidin-3-yl)-3,5-difluorophenyl)piperidin-4-yl)acetaldehyde (22.5 mg, 0.065 mmol) in DCE (10 mL) was added Ti(OiPr)₄ (12 mg, 0.043 mmol). The resulting mixture was stirred at rt for 2 hour. Then Na(OAc)₃BH (13.8 mg, 0.065 mmol). The resulting mixture was stirred at rt for 16 hour. The reaction was extracted with DCM (2×10.0 mL). The combined organic layer was washed with brine (2×10.0 mL), dried over Na₂SO₄ and concentrated under vacuum to afford the crude residue, which was purified by prep-HPLC with C-18 column chromatography (0.1% FA in water: acetonitrile=90: 10˜ 60: 40 gradient elution) to give the title product (10 mg, 23%). ¹H NMR (500 MHz, DMSO) δ 12.62 (s, 1H), 10.79 (s, 1H), 8.78 (d, J=8.7 Hz, 3H), 8.20 (s, 2H), 7.86 (d, J=9.3 Hz, 1H), 7.30 (s, 1H), 6.70 (s, 1H), 6.53 (d, J=12.8 Hz, 2H), 3.96 (dd, J=12.6, 4.9 Hz, 1H), 3.71 (s, 3H), 3.67 (d, J=12.3 Hz, 2H), 3.03 (d, J=11.2 Hz, 2H), 2.95 (d, J=10.7 Hz, 2H), 2.86-2.57 (m, 12H), 2.52 (d, J=10.5 Hz, 2H), 2.06 (d, J=12.8 Hz, 3H), 1.96 (d, J=14.4 Hz, 6H), 1.90 (d, J=8.6 Hz, 3H), 1.81 (s, 1H), 1.65-1.62 (m, 5H), 1.45 (d, J=11.5 Hz, 1H), 1.25-1.06 (m, 4H); [M+H]⁺=1026.3.

### Example 267: 3-(4-(4-(2-((1R,4R)-5-(1-(4-((5-bromo-4-((5-(dimethylphosphoryl)quinoxalin-6-yl)amino)pyrimidin-2-yl)amino)-5-methoxy-2-methylphenyl)piperidin-4-yl)-2,5-diazabicyclo[2.2.1]heptan-2-yl)ethyl)piperidin-1-yl)-2,6-difluorophenyl)piperidine-2,6-dione

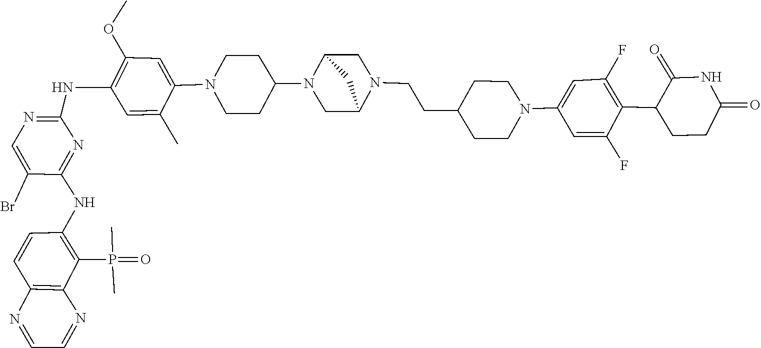 [SMILES: COc1cc(N2CCC(N3C[C@H]4C[C@@H]3CN4CCC3CCN(c4cc(F)c(C5CCC(=O)NC5=O)c(F)c4)CC3)CC2)c(C)cc1Nc1ncc(Br)c(Nc2ccc3nccnc3c2P(C)(C)=O)n1]

The titled compound was prepared in a manner similar to that in Example 266. ¹H NMR (500 MHz, DMSO) δ 12.69 (s, 1H), 10.86 (s, 1H), 8.86 (dd, J=9.7, 1.8 Hz, 3H), 8.31-8.21 (m, 2H), 7.92 (d, J=9.5 Hz, 1H), 7.36 (s, 1H), 6.76 (s, 1H), 6.61 (d, J=12.8 Hz, 2H), 4.04 (dd, J=12.6, 5.0 Hz, 1H), 3.78 (s, 3H), 3.74 (d, J=13.2 Hz, 2H), 3.55 (s, 1H), 3.02 (s, 2H), 2.86-2.51 (m, 14H), 2.09 (s, 3H), 2.02 (d, J=14.4 Hz, 6H), 1.94 (dd, J=16.7, 9.1 Hz, 2H), 1.82 (d, J=9.8 Hz, 1H), 1.72 (d, J=11.7 Hz, 2H), 1.65 (d, J=8.5 Hz, 1H), 1.58 (d, J=8.4 Hz, 2H), 1.49 (d, J=12.2 Hz, 3H), 1.35 (d, J=7.0 Hz, 2H), 1.25-1.13 (m, 2H). [M+H]⁺=1026.3.

### Example 268: 3-(4-(4-(2-((1S,4S)-5-(1-(4-((5-bromo-4-((5-(dimethylphosphoryl)quinoxalin-6-yl)amino)pyrimidin-2-yl)amino)-5-methoxy-2-methylphenyl)piperidin-4-yl)-2,5-diazabicyclo[2.2.1]heptan-2-yl)ethyl)piperidin-1-yl)-2,6-difluorophenyl)piperidine-2,6-dione

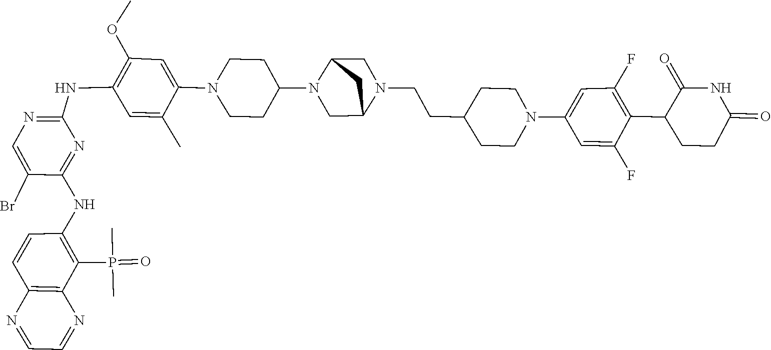 [SMILES: COc1cc(N2CCC(N3C[C@@H]4C[C@H]3CN4CCC3CCN(c4cc(F)c(C5CCC(=O)NC5=O)c(F)c4)CC3)CC2)c(C)cc1Nc1ncc(Br)c(Nc2ccc3nccnc3c2P(C)(C)=O)n1]

The titled compound was prepared in a manner similar to that in Example 266. ¹H NMR (500 MHz, DMSO) δ 12.69 (s, 1H), 10.86 (s, 1H), 8.86 (dd, J=9.8, 1.8 Hz, 3H), 8.30-8.25 (m, 2H), 7.92 (d, J=9.6 Hz, 1H), 7.36 (s, 1H), 6.76 (s, 1H), 6.61 (d, J=12.8 Hz, 2H), 4.04 (dd, J=12.4, 5.0 Hz, 1H), 3.78 (s, 3H), 3.74 (d, J=13.2 Hz, 2H), 3.57 (s, 1H), 3.03 (d, J=7.0 Hz, 2H), 2.85-2.56 (m, 14H), 2.09 (s, 3H), 2.02 (d, J=14.4 Hz, 6H), 1.94 (dd, J=15.0, 9.7 Hz, 2H), 1.82 (d, J=10.8 Hz, 1H), 1.72 (d, J=11.9 Hz, 3H), 1.60 (d, J=9.0 Hz, 2H), 1.50 (s, 3H), 1.36 (d, J=6.9 Hz, 2H), 1.23-1.14 (m, 2H). [M+H]⁺=1026.3.

### Example 269: 3-(4-(4-(2-(4-(1-(4-((5-chloro-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

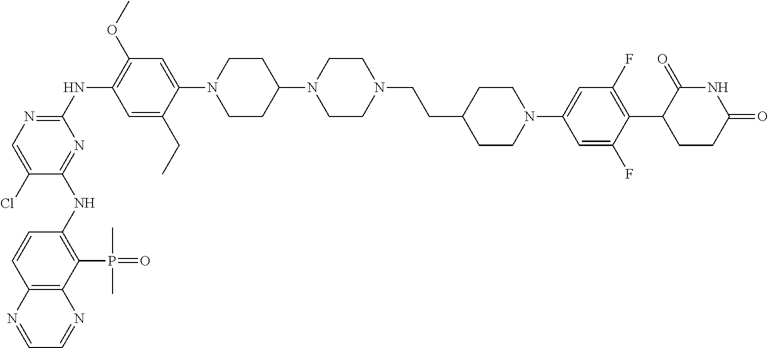 [SMILES: CCc1cc(Nc2ncc(Cl)c(Nc3ccc4nccnc4c3P(C)(C)=O)n2)c(OC)cc1N1CCC(N2CCN(CCC3CCN(c4cc(F)c(C5CCC(=O)NC5=O)c(F)c4)CC3)CC2)CC1]

The titled compound was prepared in a manner similar to that in Example 200. ¹H NMR (500 MHz, DMSO) δ 12.91 (s, 1H), 10.80 (s, 1H), 8.92 (s, 1H), 8.81 (d, J=17.1 Hz, 2H), 8.45 (s, 1H), 8.16 (s, 1H), 7.84 (d, J=8.7 Hz, 1H), 7.35 (s, 1H), 6.77 (s, 1H), 6.57 (d, J=12.9 Hz, 2H), 3.98 (dd, J=12.5, 4.9 Hz, 2H), 3.72 (s, 6H), 3.07 (d, J=10.6 Hz, 6H), 2.78-2.62 (m, 6H), 2.51-2.44 (m, 4H), 2.08-1.86 (m, 12H), 1.72-1.68 (m 4H), 1.60-1.37 (m, 4H), 1.20-1.10 (m, 2H), 0.93 (t, J=7.3 Hz, 3H). [M+H]⁺=984.4.

### Example 270: 3-(4-(4-(2-(4-(1-(4-((5-bromo-4-((5-(dimethylphosphoryl)-2-methyl-1-oxo-1,2-dihydrophthalazin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

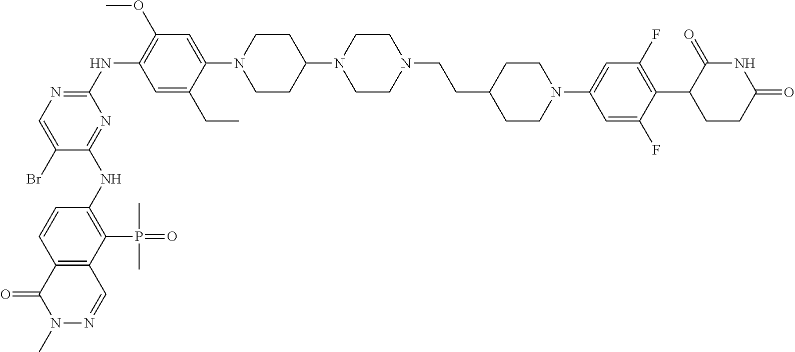 [SMILES: CCc1cc(Nc2ncc(Br)c(Nc3ccc4c(=O)n(C)ncc4c3P(C)(C)=O)n2)c(OC)cc1N1CCC(N2CCN(CCC3CCN(c4cc(F)c(C5CCC(=O)NC5=O)c(F)c4)CC3)CC2)CC1]

The titled compound was prepared in a manner similar to that in Example 200. 1H NMR (500 MHz, DMSO) δ 12.20 (s, 1H), 10.86 (s, 1H), 8.57 (s, 2H), 8.26-8.14 (m, 3H), 7.36 (s, 1H), 6.75 (s, 1H), 6.60 (d, J=13.0 Hz, 2H), 4.04 (dd, J=12.1, 4.8 Hz, 1H), 3.75 (s, 3H), 3.72 (s, 3H), 2.98 (d, J=9.3 Hz, 3H), 2.80-2.59 (m, 8H), 2.41 (d, J=6.9 Hz, 6H), 2.31 (d, J=7.1 Hz, 4H), 2.12-1.93 (m, 9H), 1.84 (d, J=10.8 Hz, 2H), 1.71 (d, J=12.0 Hz, 2H), 1.56 (d, J=9.7 Hz, 4H), 1.38 (d, J=6.8 Hz, 2H), 1.17 (d, J=11.3 Hz, 2H), 0.91 (s, 3H); [M+H]⁺=1058.4.

### Example 272: 5-(4-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)piperidin-1-yl)-2-(2,6-dioxopiperidin-3-yl)isoindoline-1,3-dione

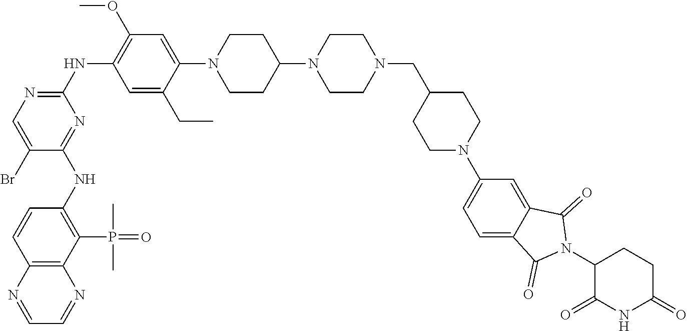 [SMILES: CCc1cc(Nc2ncc(Br)c(Nc3ccc4nccnc4c3P(C)(C)=O)n2)c(OC)cc1N1CCC(N2CCN(CC3CCN(c4ccc5c(c4)C(=O)N(C4CCC(=O)NC4=O)C5=O)CC3)CC2)CC1]

The titled compound was prepared in a manner similar to that in Example 59. ¹H NMR (400 MHz, DMSO) δ 12.64 (s, 1H), 11.06 (s, 1H), 8.86 (d, J=4.8 Hz, 3H), 8.27 (s, 2H), 7.89 (s, 1H), 7.64 (d, J=8.7 Hz, 1H), 7.37 (s, 1H), 6.91 (s, 1H), 6.81 (s, 2H), 5.04 (s, 1H), 3.77 (s, 3H), 3.61 (m, 4H), 3.03 (s, 6H), 2.88 (s, 2H), 2.69 (d, J=11.4 Hz, 3H), 2.36 (m, 9H), 2.17 (s, 2H), 2.02 (d, J=14.4 Hz, 8H), 1.85 (s, 2H), 1.60 (s, 6H), 0.92 (s, 3H). [M+H]⁺=1049.9

### Example 274: 3-(4-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-2-oxoethyl)piperidin-1-yl)-2,6-difluorophenyl)piperidine-2,6-dione

### Step 1: ethyl 2-(1-(4-(2,6-bis(benzyloxy)pyridin-3-yl)-3,5-difluorophenyl)piperidin-4-yl)acetate

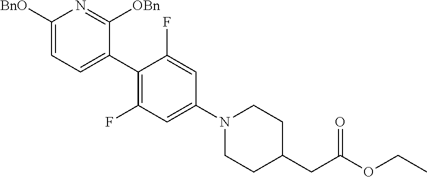 [SMILES: CCOC(=O)CC1CCN(c2cc(F)c(-c3ccc(OCc4ccccc4)nc3OCc3ccccc3)c(F)c2)CC1]

A mixture of 2,6-bis(benzyloxy)-3-(4-bromo-2,6-difluorophenyl)pyridine (2.0 g, 4.158 mmol) and ethyl 2-(piperidin-4-yl)acetate (711.0 mg, 4.158 mmol), Cs₂CO₃ (2.0 g, 6.237 mmol), XPhos(396.7 mg, 0.832 mmol), Pd₂(dba)₃ (380.5 mg, 0.416 mmol) in dioxane (20 mL) was stirred overnight at 110° C. under nitrogen atmosphere. The resulting mixture was concentrated under reduced pressure. The residue was purified by silica gel column chromatography, eluted with PE/EtOAc (1:1) to afford the product (1.0 g, 42.1%) [M+1]⁺=573.3.

### Step 2: 2-(1-(4-(2,6-bis(benzyloxy)pyridin-3-yl)-3,5-difluorophenyl)piperidin-4-yl)acetic acid

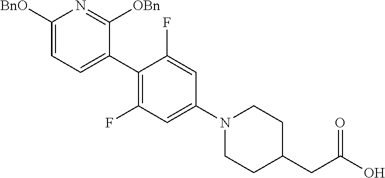 [SMILES: O=C(O)CC1CCN(c2cc(F)c(-c3ccc(OCc4ccccc4)nc3OCc3ccccc3)c(F)c2)CC1]

Into a 100-mL round-bottom flask, was placed ethyl 2-(1-(4-(2,6-bis(benzyloxy)pyridin-3-yl)-3,5-difluorophenyl)piperidin-4-yl)acetate (1.0 g, 1.748 mmol), EtOH (20 mL), THF (8 mL), H₂O (4 mL), NaOH (280.0 mg, 7.000 mmol). The resulting solution was stirred overnight at room temperature. The pH value of the solution was adjusted to <7 with 1N HCl (aq). The resulting mixture was concentrated under vacuum. The resulting mixture was extracted with EtOAc (200 mL) and the organic layers combined and dried over anhydrous sodium sulfate and concentrated under vacuum. The residue was purified by silica gel column chromatography, eluted with PE/EtOAc (1:1) to afford the product (501 mg, 52%). [M+1]⁺=545.3.

### Step 3: 2-(1-(4-(2,6-dioxopiperidin-3-yl)-3 ,5-difluorophenyl)piperidin-4-yl)acetic acid

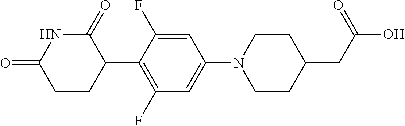 [SMILES: O=C(O)CC1CCN(c2cc(F)c(C3CCC(=O)NC3=O)c(F)c2)CC1]

A mixture of (1-(4-(2,6-bis(benzyloxy)pyridin-3-yl)-3,5-difluorophenyl)piperidin-4-yl)acetic acid (500.0 mg, 0.919 mmol) and Pd/C (300.0 mg) in EtOH (10 mL) and DCM (2 mL) was stirred overnight at 45° C. under hydrogen atmosphere. The resulting mixture was filtered, the filter cake was washed with MeOH (50 mL) and DCM (50 mL). The filtrate was concentrated under reduced pressure to afford the product (282.2 mg, 84.0%) was obtained. [M+1]⁺=367.0.

### Step 4: 3-(4-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-2-oxoethyl)piperidin-1-yl)-2,6-difluorophenyl)piperidine-2,6-dione

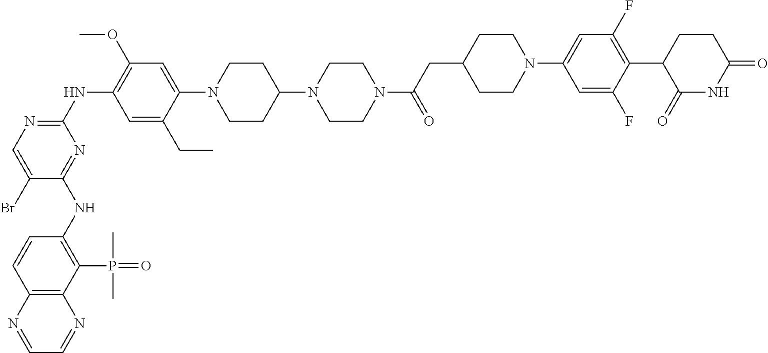 [SMILES: CCc1cc(Nc2ncc(Br)c(Nc3ccc4nccnc4c3P(C)(C)=O)n2)c(OC)cc1N1CCC(N2CCN(C(=O)CC3CCN(c4cc(F)c(C5CCC(=O)NC5=O)c(F)c4)CC3)CC2)CC1]

The titled compound (14 mg, 26%) was prepared in a manner similar to that in Example 208 step 8 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 2-(1-(4-(2,6-dioxopiperidin-3-yl)-3,5-difluorophenyl)piperidin-4-yl)acetic acid. ¹H NMR (400 MHz DMSO) δ 12.64 (s, 1H), 10.87 (s, 1H), 8.86 (d, J=5.2 Hz, 3H), 8.27 (s, 2H), 7.91 (s, 1H), 7.37 (s, 1H), 6.81 (s, 1H), 6.61 (d, J=13.3 Hz, 2H), 4.05 (s, 1H), 3.77 (s, 6H), 3.47 (s, 4H), 3.01 (s, 7H), 2.73 (d, J=12.1 Hz, 4H), 2.50 (s, 3H), 2.27 (s, 2H), 2.02 (d, J=14.2 Hz, 4H), 2.02 (d, J=14.2 Hz, 2H), 1.84 (s, 3H), 1.71 (s, 2H), 1.61 (s, 2H), 1.23 (s, 4H), 0.92 (s, 3H). [M+H]⁺=1042.1.

### Example 280: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)phenyl)-3-methylpiperidine-2,6-dione

### Step 1: ethyl 4-(4-bromophenyl)-4-cyanopentanoate

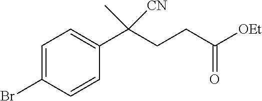 [SMILES: CCOC(=O)CCC(C)(C#N)c1ccc(Br)cc1]

To a solution of 2-(4-bromophenyl)propanenitrile (4 g, 19 mmol) in THF(50 mL) was added LDA (2M, 10.5 mL) dropwise in 10 min at −65° C., the reaction solution was stirred for 30 min at this temperature, then to this was added ethyl 3-bromopropanoate (3.8 g, 21 mmol) in THF(10 mL) dropwise in 10 min. The resulting solution was stirred for 30 min at −65° C., then allowed the temperature rise to room temperature naturally. The reaction was quenched by the addition of sat.aq. NH₄Cl solution, extracted with EtOAc (50 mL×3), the combined organic layer and washed with brine, dried over anhydrous Na₂SO₄, after filtration, the filtrate was concentrated under reduced pressure to afford product (5.1 g, 86.4%). [M+H]⁺=310.1.

### Step 2: 4-(4-bromophenyl)-4-cyanopentanoic acid

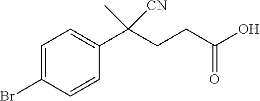 [SMILES: CC(C#N)(CCC(=O)O)c1ccc(Br)cc1]

To a solution of ethyl 4-(4-bromophenyl)-4-cyanopentanoate (3.1 g, 10 mmol) in THF/H₂O (30 mL/10 mL) was added LiOH (720 mg, 30 mmol). The reaction mixture was stirred for 3 h at room temperature. The resulting mixture was diluted with water, extracted with EtOAc (20 mL×2). The pH value of water phase was adjusted to 4-5, extracted with EtOAc (30 mL×2), The combined organic layers were washed with brine (50 mL), dried over anhydrous Na₂SO₄. After filtration, the filtrate was concentrated under reduced pressure to afford product (2.6 g, 92.2%). [M+H]⁺=282.0.

### Step 3: 3-(4-bromophenyl)-3-methylpiperidine-2,6-dione

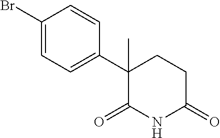 [SMILES: CC1(c2ccc(Br)cc2)CCC(=O)NC1=O]

To a solution of 4-(4-bromophenyl)-4-cyanopentanoic acid (2.6 g, 9.2 mmol) in toluene (20 mL) was added conc. H₂SO₄(0.6 mL, 10.1 mmol). The resulting solution was stirred at 100° C. for 3 h. The reaction mixture was concentrated under vacuum, then the mixture was poured into water, the pH value was adjusted to 7-8 with sat. aq. NaHCO₃ solution, extracted with EtOAc (30 mL×3). The organic layer was washed with water and brine, dried over anhydrous Na₂SO₄. The solvent was evaporated to dryness to afford product (2.1 g, 80.8%). [M+H]⁺=282.0.

### Step 4: (E)-3-(4-(2-ethoxyvinyl)phenyl)-3-methylpiperidine-2,6-dione

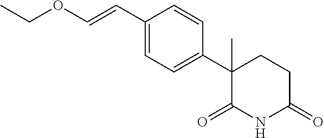 [SMILES: CCO/C=C/c1ccc(C2(C)CCC(=O)NC2=O)cc1]

To a stirred solution of 3-(4-bromophenyl)-3-methylpiperidine-2,6-dione (200 mg, 0.71 mmol) and (E)-2-(2-ethoxyvinyl)-4,4,5,5-tetramethyl-1,3,2-dioxaborolane (169 mg, 0.85 mmol) in DMF/H₂O (8 mL/2 mL) were added Pd(dtbpf)Cl₂ (46 mg, 0.071 mmol) and CsF (216 mg, 1.4 mmol). The resulting mixture was stirred for 2 h at 80° C. under nitrogen atmosphere. The reaction solution was diluted with water, extracted with EtOAc (10 mL×3). The organic layer was washed with water and brine, dried over anhydrous Na₂SO₄. The solvent was evaporated to dryness and the crude residue was purified by a silica gel column, eluted with PE/EtOAc=1:1 to afford product. (190 mg, 97.9%). [M+H]⁺=274.1.

### Step 5: 2-(4-(3-methyl-2,6-dioxopiperidin-3-yl)phenyl)acetaldehyde

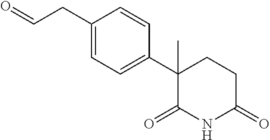 [SMILES: CC1(c2ccc(CC=O)cc2)CCC(=O)NC1=O]

(E)-3-(4-(2-ethoxyvinyl)phenyl)-3-methylpiperidine-2,6-dione (190 mg, 0.7 mmol) was dissolved in HCOOH (3 mL). The resulting solution was stirred for 2 h at room temperature. The reaction solution was evaporated to dryness to afford the product (160 mg, 92.9%) which was used directly in the next step without further purification. m/z[M+H]⁺=246.1.

### Step 6: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)phenyl)-3-methylpiperidine-2,6-dione

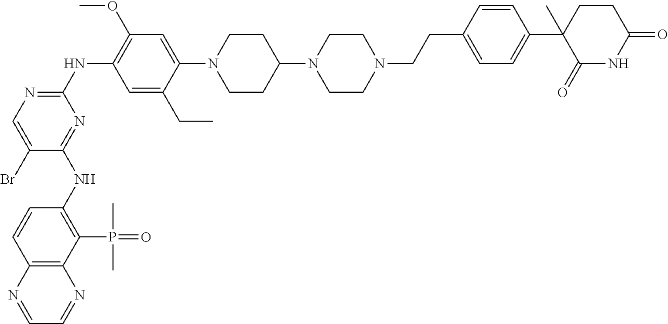 [SMILES: CCc1cc(Nc2ncc(Br)c(Nc3ccc4nccnc4c3P(C)(C)=O)n2)c(OC)cc1N1CCC(N2CCN(CCc3ccc(C4(C)CCC(=O)NC4=O)cc3)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (40 mg, 0.058 mmol), 2-(4-(3-methyl-2,6-dioxopiperidin-3-yl)phenyl)acetaldehyde (21 mg, 0.086 mmol) and NaBH(OAc)₃ (11 mg, 0.17 mmol) in DCE (3 mL) was stirred in a round bottom flask at room temperature for 2 hours. The reaction was diluted with DCM, washed with water(5 mL) and brine (5 mL), dried over Na₂SO₄ and concentrated under vacuum to afford the crude product, which was purified with pre-HPLC (0.1% FA in water: acetonitrile=90:1˜50:50 gradient elution) to give the title product (18.5 mg, 34.6%). ¹H NMR (500 MHz, DMSO-d₆) δ 12.64 (s, 1H), 10.90 (s, 1H), 8.82-8.87 (m, 3H), 8.23-8.27 (m, 2H), 7.90 (d, J=8.6 Hz, 1H), 7.38 (s, 1H), 7.18-7.23 (m, 4H), 6.81 (s, 1H), 3.77 (s, 3H), 2.97-3.04 (m, 2H), 2.66-2.75 (m, 4H), 2.54-2.61 (m, 6H), 2.39-2.49 (m, 6H), 2.27-2.38 (m, 3H), 1.97-2.13 (m, 8H), 1.82-1.90 (m, 2H), 1.53-1.63 (m, 2H), 1.42 (s, 3H), 0.93 (t, J=6.9 Hz, 3H). [M+H]⁺=923.3.

### Example 281: 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)pyrrolidin-1-yl)phenyl)piperidine-2,6-dione

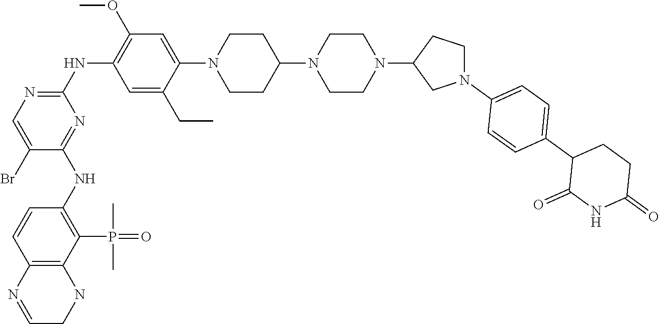

The titled compound was prepared in a manner similar to that in Example 200. ¹H NMR (500 MHz, DMSO-d₆)⁶H 12.58 (s, 1H), 10.67 (s, 1H), 8.76-8.80 (m, 3H), 8.20 (s, 2H), 7.84 (d, J=8.6 Hz, 1H), 7.31 (s, 1H), 6.93 (d, J=8.2 Hz, 2H), 6.75 (s, 1H), 6.43 (d, J=8.3 Hz, 2H), 3.58-3.72 (m, 5H), 3.35-3.40 (m, 2H), 3.11-3.17 (m, 2H), 2.77-2.98 (m, 5H), 2.47-2.69 (m, 8H), 2.20-2.30 (m, 3H), 2.01-2.15 (m, 2H), 1.95 (d, J=14.3 Hz, 8H), 1.70-1.84 (m, 3H), 1.46-1.57 (m, 2H), 0.86 (s, 3H).[M+H]⁺=950.4.

### Example 282: 3-(4-(4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)butyl)-2,6-difluorophenyl)piperidine-2,6-dione

### Step 1: 4-(4-bromo-3,5-difluorophenyl)but-3-yn-1-ol

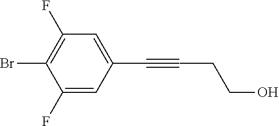 [SMILES: OCCC#Cc1cc(F)c(Br)c(F)c1]

A mixture of 2-bromo-1,3-difluoro-5-iodobenzene (12 g, 37.6 mmol), but-3-yn-1-ol (3.95 g, 56.4 mmol), Pd(PPh₃)₂Cl₂ (2.6 g, 3.76 mmol) and CuI (715 mg, 3.76 mmol) in Et₃N/DMF (40 mL/20 mL) was stirred in a flask at 80° C. overnight. The reaction mixture was allowed to cool down to room temperature.

The resulting mixture was diluted with water and extracted with EtOAc (3×80 mL). The combined organic layers were washed with brine (100 mL), dried over anhydrous Na₂SO₄. After filtration, the filtrate was concentrated under reduced pressure. The residue was purified by silica gel column chromatography, eluted with PE/EtOAc (3: 1) to afford the product (7 g, 71.3%). [M+H]⁺=261.0.

### Step 2: 4-(4-(2,6-bis(benzyloxy)pyridin-3-yl)-3,5-difluorophenyl)but-3-yn-1-ol

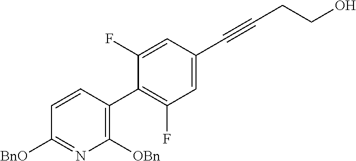 [SMILES: OCCC#Cc1cc(F)c(-c2ccc(OCc3ccccc3)nc2OCc2ccccc2)c(F)c1]

A mixture of 4-(4-bromo-3,5-difluorophenyl)but-3-yn-1-ol (7 g, 26.8 mmol), 2,6-bis(benzyloxy)-3-(4,4,5,5-tetramethyl-1,3,2-dioxaborolan-2-yl)pyridine (11.2 g, 26.8 mmol), Pd(dtbpf)Cl₂ (877 mg, 1.34 mmol) and CsF (8.2 g, 53.6 mmol) in DMF (80 mL) and water (20 mL) was stirred in a flask at 90° C. under nitrogen atmosphere for 16 hrs. The reaction mixture was allowed to cool down to room temperature. The resulting mixture was extracted with EtOAc (3×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EtOAc (2: 1) to afford the product (7 g, 55.3%). [M+H]⁺=472.2.

### Step 3: 3-(2,6-difluoro-4-(4-hydroxybutyl)phenyl)piperidine-2,6-dione

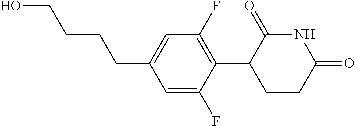 [SMILES: O=C1CCC(c2c(F)cc(CCCCO)cc2F)C(=O)N1]

To a stirred solution of 4-(4-(2,6-bis(benzyloxy)pyridin-3-yl)-3,5-difluorophenyl)but-3-yn-1-ol (7 g, 14.8 mmol) in MeOH (100 mL) and DCM (20.00 mL) was added Pd/C (wet, 10%) (1 g) under nitrogen atmosphere. The resulting mixture was stirred for 16 hrs at room temperature under hydrogen atmosphere. The resulting mixture was filtered, the filter cake was washed with DCM/CH₃OH (10: 1, 50 mL). The filtrate was concentrated under reduced pressure to afford the product (3.9 g, 88.6%). [M+H]⁺=298.1.

### Step 4: 4-(4-(2,6-dioxopiperidin-3-yl)-3,5-difluorophenyl)butanal

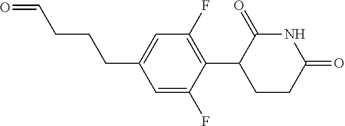 [SMILES: O=CCCCc1cc(F)c(C2CCC(=O)NC2=O)c(F)c1]

To a solution of 3-(2,6-difluoro-4-(4-hydroxybutyl)phenyl)piperidine-2,6-dione (200 mg, 0.67 mmol) in DMSO (3 mL) was added IBX (564 mg, 2 mmol). The resulting solution was stirred at 30° C. under nitrogen atmosphere for 3 hrs. The reaction mixture was quenched with water, extracted with DCM (3×10 mL). The combined organic layers were washed with brine (10 mL), dried over anhydrous Na₂SO₄. After filtration, the filtrate was concentrated under reduced pressure to afford the product (120 mg, 60.6%), [M+H]⁺=296.1.

### Step 5: 3-(4-(4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)butyl)-2,6-difluorophenyl)piperidine-2,6-dione

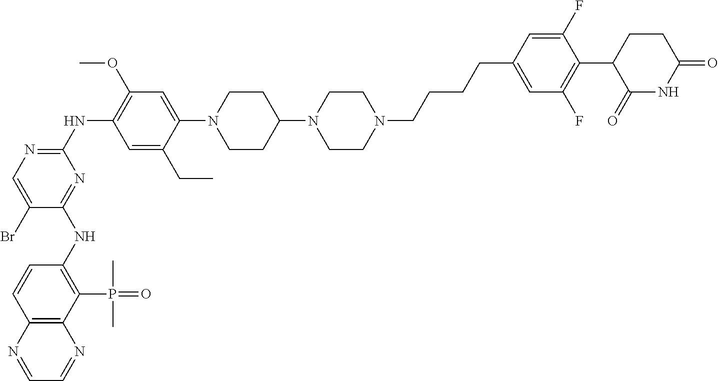 [SMILES: CCc1cc(Nc2ncc(Br)c(Nc3ccc4nccnc4c3P(C)(C)=O)n2)c(OC)cc1N1CCC(N2CCN(CCCCc3cc(F)c(C4CCC(=O)NC4=O)c(F)c3)CC2)CC1]

The titled compound (12 mg, 27%) was prepared in a manner similar to that in Example 204 step 6 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 4-(4-(2,6-dioxopiperidin-3-yl)-3,5-difluorophenyl)butanal. ¹H NMR (500 MHz, DMSO-d₆)⁶H 12.64 (s, 1H), 10.94 (s, 1H), 8.81-8.87 (m, 3H), 8.29 (s, 2H), 7.92 (t, J=15.5 Hz, 1H), 7.38 (s, 1H), 6.98 (d, J=10.0 Hz, 2H), 6.81 (s, 1H), 4.20 (dd, J=12.7, 5.0 Hz, 1H), 3.77 (s, 3H), 3.01 (d, J=11.1 Hz, 2H), 2.87-2.75 (m, 1H), 2.71 (dd, J=19.8, 8.4 Hz, 2H), 2.53-2.62 (m, 6H), 2.26-2.47 (m, 10H), 2.13 (dd, J=26.0, 13.1, 3.8 Hz, 1H), 2.01 (t, J=11.5 Hz, 7H), 1.85 (d, J=10.9 Hz, 2H), 1.53-1.61 (m, 4H), 1.40-1.46 (m, 2H), 0.92 (t, J=7.1 Hz, 3H). [M+H]⁺=973.3.

### Example 284: 3-((4-((1r,3r)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)cyclobutyl)phenyl)amino)piperidine-2,6-dione

### Step 1: 3-((benzyloxy)methyl)cyclobutan-1-ol

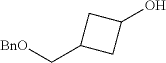 [SMILES: OC1CC(COCc2ccccc2)C1]

Into a 250 mL round-bottom flask was added 3-((benzyloxy)methyl)cyclobutan-1-one (5.0 g, 26.28 mmol) in MeOH (80 mL) at room temperature. NaBH₄ (1.49 g, 39.42 mmol) was added in portions at 0° C. The resulting mixture was stirred for 2 h at room temperature. The resulting mixture was extracted with EtOAc (400 mL). The combined organic layers were washed with brine (80 mL), dried over anhydrous Na₂SO₄. After filtration, the filtrate was concentrated under reduced pressure. The residue was purified by silica gel column chromatography, eluted with PE/EtOAc=1:1 to afford the product (4.9 g, 96.97%). [M+H]⁺=193.1.

### Step 2: 3-((benzyloxy)methyl)cyclobutyl 4-methylbenzenesulfonate

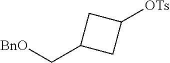 [SMILES: Cc1ccc(S(=O)(=O)OC2CC(COCc3ccccc3)C2)cc1]

Into a 250 mL 3-necked round-bottom flask were added 3-((benzyloxy)methyl)cyclobutan-1-ol (4.9 g, 25.48 mmol), DMAP (0.62 g, 5.07 mmol), TEA (3.09 g, 30.59 mmol), DCM (50 mL) at room temperature. To a stirred solution was added p-toluenesulfonyl chloride (5.34 g, 27.95 mmol) in portions at 0° C. under nitrogen atmosphere. The resulting mixture was stirred for 2 h at room temperature under nitrogen atmosphere. The reaction was quenched with water at 0° C. The mixture was acidified to pH=4 with HCl (1 N). The aqueous layer was extracted with DCM (500 mL), dried over anhydrous Na₂SO₄. After filtration, the filtrate was concentrated under reduced pressure. The residue was purified by silica gel column chromatography, eluted with PE/EtOAc (9%) to afford the product (6.5 g, 73.62%). [M+H]⁺=347.4.

### Step 3: (((3-iodocyclobutyl)methoxy)methyl)benzene

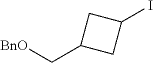 [SMILES: IC1CC(COCc2ccccc2)C1]

To a stirred solution of 3-((benzyloxy)methyl)cyclobutyl 4-methylbenzenesulfonate (6.30 g, 18.18 mmol) and NaI (8.18 g, 54.5 mmol) in DMSO (120 mL) at room temperature under air atmosphere. The resulting mixture was stirred overnight at 120° C. under nitrogen atmosphere. The resulting mixture was extracted with EtOAc (500 mL). The combined organic layers were washed with brine (300 mL), dried over anhydrous Na₂SO₄. After filtration, the filtrate was concentrated under reduced pressure. The residue was purified by silica gel column chromatography, eluted with PE/EtOAc (1:1) to afford the product (4.0 g, 72.8%). 1H NMR (400 MHz, DMSO-d6) δ ppm 7.40-7.24 (m, 5H), 4.53 (J=9.3, 7.5 Hz, 2H), 4.48 (s, 1H), 4.47 (s, 1H), 3.51-3.41 (m, 2H), 2.84-2.71 (m, 1H), 2.75-2.68 (m, 1H), 2.68-2.55 (m, 1H), 2.59-2.52 (m, 1H).

### Step 4: tert-butyl (4-(3-((benzyloxy)methyl)cyclobutyl)phenyl)carbamate

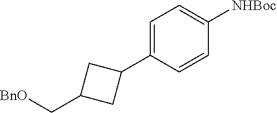 [SMILES: CC(C)(C)OC(=O)Nc1ccc(C2CC(COCc3ccccc3)C2)cc1]

Into a 250 mL 3-necked round-bottom flask were added tert-butyl N-(4-iodophenyl)carbamate (4.0 g, 12.534 mmol), (((3-iodocyclobutyl)methoxy)methyl)benzene (5.68 g, 18.75 mmol), NaI (0.47 g, 3.13 mmol), Zn (1.64 g, 25.23 mmol), NiCl₂(DME) (0.28 g, 1.27 mmol), 1H-Imidazole-4-carbonitrile (0.12 g, 1.29 mmol), DMA (50 mL) and TFA (0.14 g, 1.22 mmol) at room temperature. The resulting mixture was stirred overnight at 60° C. under nitrogen atmosphere. The resulting mixture was extracted with EtOAc (300 mL). The combined organic layers were washed with brine (200 mL), dried over anhydrous Na₂SO₄. After filtration, the filtrate was concentrated under reduced pressure. The residue was purified by silica gel column chromatography, eluted with PE/EtOAc (3:1) to afford the product (1.2 g, 26.05%). [M+H]⁺=368.3.

### Step 5: 4-(3-((benzyloxy)methyl)cyclobutyl) aniline

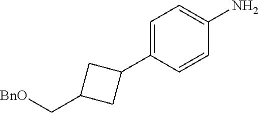 [SMILES: Nc1ccc(C2CC(COCc3ccccc3)C2)cc1]

Into a 100 mL round-bottom flask were added tert-butyl (4-(3-((benzyloxy)methyl)cyclobutyl)phenyl)carbamate (1.20 g, 3.26 mmol) and 4 M HCl in 1,4-dioxane (15 mL) at room temperature. The resulting mixture was stirred for 3 h at room temperature under air atmosphere. The resulting mixture was concentrated under vacuum. The residue was purified by reverse flash chromatography with the following conditions: column, C18 silica gel; mobile phase, ACN in water, 10% to 50% gradient in 10 min; detector, UV 254 nm to obtain the product (700 mg, 80.18%). [M+H]⁺=268.2.

### Step 6: 3-((4-(3-((benzyloxy)methyl)cyclobutyl)phenyl)amino)piperidine-2,6-dione

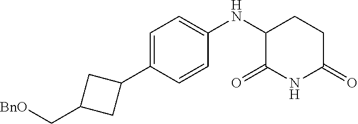 [SMILES: O=C1CCC(Nc2ccc(C3CC(COCc4ccccc4)C3)cc2)C(=O)N1]

To a stirred solution/mixture of 4-(3-((benzyloxy)methyl)cyclobutyl) aniline (700.0 mg, 2.61 mmol) and 3-bromopiperidine-2,6-dione (502.7 mg, 2.61 mmol) in DMF (7 mL) was added DIEA (1015 mg, 7.85 mmol) in portions at room temperature under air atmosphere. The resulting mixture was stirred overnight at 80° C. under nitrogen atmosphere. The resulting mixture was extracted with EtOAc (200 mL). The combined organic layers were washed with brine (100 mL), dried over anhydrous Na₂SO₄. After filtration, the filtrate was concentrated under reduced pressure. The residue was purified by Prep-TLC with PE/EtOAc (1:1) to afford the product (650 mg, 65.60%). [M+H]⁺=379.2.

### Step 5: 3-((4-((1r,3r)-3-(hydroxymethyl)cyclobutyl)phenyl)amino)piperidine-2,6-dione

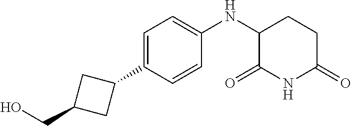 [SMILES: O=C1CCC(Nc2ccc([C@H]3C[C@H](CO)C3)cc2)C(=O)N1]

Into a 50 mL round-bottom flask were added 3-((4-(3-((benzyloxy)methyl)cyclobutyl)phenyl)amino)piperidine-2,6-dione (650.0 mg, 1.71 mmol) and Pd/C (650.0 mg, 10% wt) in a mixture of EtOH (5 mL) and THF (5 mL) at room temperature. The resulting mixture was stirred overnight at room temperature under hydrogen atmosphere. The resulting mixture was diluted with MeOH/DCM (100 mL). The resulting mixture was concentrated under vacuum. The crude product was purified by Prep-HPLC with the following conditions (Column: XBridge Prep OBD C18 Column, 19*250 mm, 5 μm; Mobile Phase A: Water(0.1% FA), Mobile Phase B: MeOH-HPLC; Flow rate: 20 mL/min; Gradient: 50% B to 80% B in 8 min, 80% B; Wave Length: 254 nm to afford the product (400 mg, 80.77%). [M+H]⁺=289.2.

### Step 6: ((1r,3r)-3-(4-((2,6-dioxopiperidin-3-yl)amino)phenyl)cyclobutyl)methyl methanesulfonate

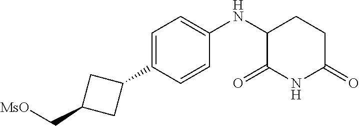 [SMILES: CS(=O)(=O)OC[C@H]1C[C@H](c2ccc(NC3CCC(=O)NC3=O)cc2)C1]

The titled compound (120 mg, 37%) was prepared in a manner similar to that in Example 207 step 4 from 3-((4-((1r,3r)-3-(hydroxymethyl)cyclobutyl)phenyl)amino)piperidine-2,6-dione and MsCl.

### Step 7: 3-((4-((1r,3r)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)cyclobutyl)phenyl)amino)piperidine-2,6-dione

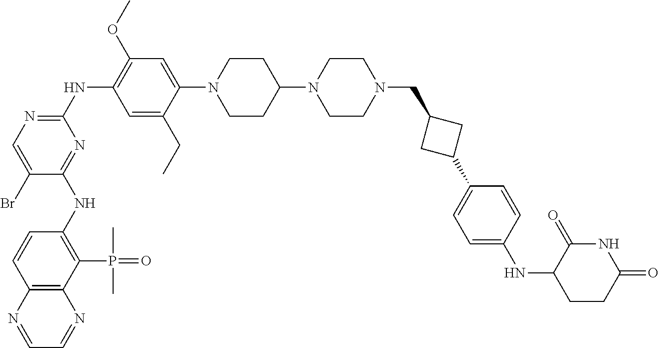

The titled compound (11 mg, 27%) was prepared in a manner similar to that in Example 207 step 5 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and ((1r,3r)-3-(4-((2,6-dioxopiperidin-3-yl)amino)phenyl)cyclobutyl)methyl methanesulfonate. ¹H NMR (500 MHz, DMSO) δ 12.64 (s, 1H), 10.76 (s, 1H), 8.85 (dt, J=19.5, 9.7 Hz, 3H), 8.27 (s, 2H), 7.90 (d, J=9.7 Hz, 1H), 7.37 (s, 1H), 6.94 (d, J=8.5 Hz, 2H), 6.81 (s, 1H), 6.61 (d, J=8.5 Hz, 2H), 5.65 (d, J=7.6 Hz, 1H), 4.31-4.23 (m, 1H), 3.77 (s, 3H), 3.23-3.14 (m, 1H), 3.01 (dd, J=10.0, 1.5 Hz, 2H), 2.80-2.65 (m, 3H), 2.65-2.52 (m, 5H), 2.47 (d, J=7.6 Hz, 2H), 2.43-2.24 (m, 11H), 2.13-2.06 (m, 1H), 2.02 (d, J=14.4 Hz, 6H), 1.91-1.79 (m, 3H), 1.68-1.51 (m, 4H), 0.92 (t, J=7.2 Hz, 3H); [M+H]⁺=964.5.

### Example 286: 3-(4-(2-((R)-4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)-3-(methoxymethyl)piperazin-1-yl)ethyl)-2-fluorophenyl)piperidine-2,6-dione

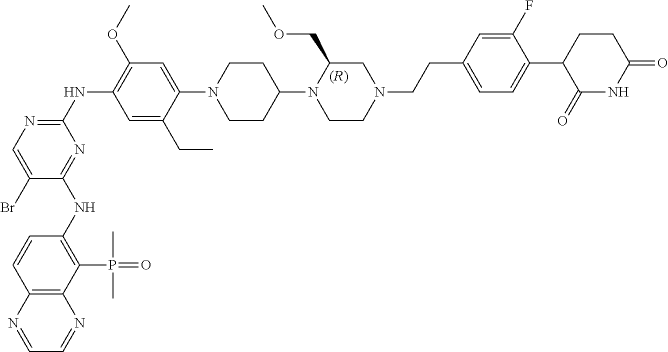

The titled compound was prepared in a manner similar to that in Example 291. 1H NMR (500 MHz, DMSO) δ 12.57 (s, 1H), 10.80 (s, 1H), 8.78 (dt, J=17.1, 8.5 Hz, 3H), 8.21 (s, 2H), 7.84 (d, J=9.0 Hz, 1H), 7.31 (s, 1H), 7.14 (t, J=7.9 Hz, 1H), 7.01 (dd, J=22.3, 9.8 Hz, 2H), 6.75 (s, 1H), 3.94 (dd, J=12.5, 4.9 Hz, 1H), 3.71 (s, 3H), 3.42 (d, J=37.3 Hz, 2H), 3.22 (s, 3H), 2.95 (d, J=9.8 Hz, 2H), 2.76 (s, 2H), 2.66 (dd, J=21.3, 8.4 Hz, 7H), 2.57-2.46 (m, 4H), 2.42-2.20 (m, 5H), 2.15-2.05 (m, 1H), 1.95 (d, J=14.4 Hz, 7H), 1.66 (dd, J=87.6, 76.5 Hz, 4H), 0.86 (t, J=6.9 Hz, 3H). [M+H]⁺=971.7

### Example 287: 3-(4-(2-((R)-4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)-2-(hydroxymethyl)piperazin-1-yl)ethyl)-2-fluorophenyl)piperidine-2,6-dione

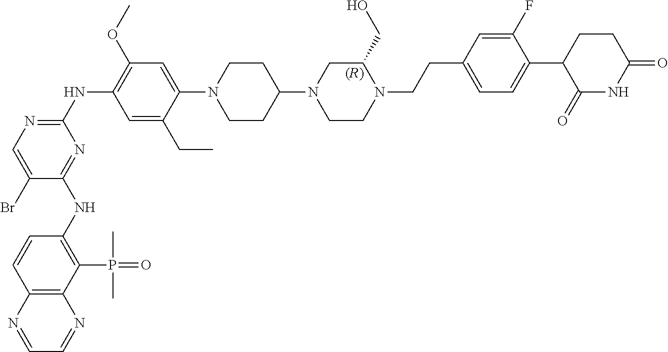

The titled compound was prepared in a manner similar to that in Example 291. 1H NMR (500 MHz, DMSO) δ 12.58 (s, 1H), 10.79 (s, 1H), 8.83-8.74 (m, 2H), 8.74-8.37 (m, 1H), 8.20 (s, 2H), 7.82 (s, 1H), 7.31 (s, 1H), 7.13 (t, J=7.9 Hz, 1H), 6.98 (dd, J=19.3, 9.8 Hz, 2H), 6.74 (s, 1H), 3.93 (dd, J=12.4, 4.9 Hz, 1H), 3.70 (s, 5H), 3.56 (dd, J=10.8, 3.8 Hz, 1H), 2.95 (d, J=10.9 Hz, 2H), 2.80 (dd, J=22.8, 10.3 Hz, 4H), 2.72-2.50 (m, 8H), 2.47 (s, 1H), 2.38 (d, J=11.6 Hz, 2H), 2.31-2.19 (m, 3H), 2.12 (d, J=9.4 Hz, 2H), 1.93 (dd, J=19.8, 9.6 Hz, 7H), 1.79 (d, J=11.0 Hz, 2H), 1.52 (d, J=11.1 Hz, 2H), 0.86 (t, J=7.0 Hz, 3H). [M+H]⁺=957.4

### Example 288: 3-(4-(2-((S)-4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)-3-(methoxymethyl)piperazin-1-yl)ethyl)-2-fluorophenyl)piperidine-2,6-dione

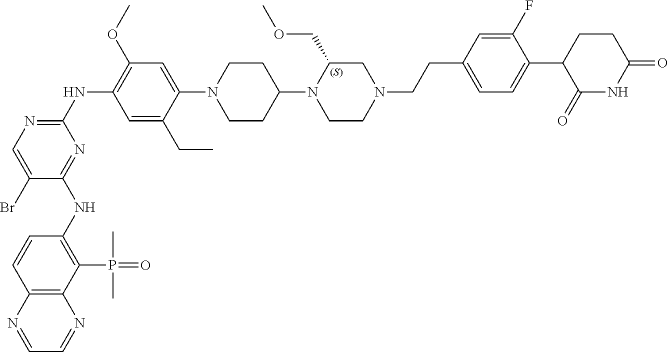

The titled compound was prepared in a manner similar to that in Example 291. 1H NMR (500 MHz, DMSO) δ 12.64 (s, 1H), 10.86 (s, 1H), 8.84 (t, J=16.7 Hz, 3H), 8.27 (s, 2H), 7.91 (d, J=8.8 Hz, 1H), 7.37 (s, 1H), 7.20 (t, J=7.9 Hz, 1H), 7.14-7.03 (m, 2H), 7.12-7.00 (m, 2H), 6.82 (s, 1H), 4.00 (dd, J=12.4, 4.8 Hz, 2H), 3.77 (s, 3H), 3.51 (d, J=4.0 Hz, 5H), 3.01 (d, J=8.5 Hz, 2H), 3.00 (s, 1H), 2.89 (s, 2H), 2.73 (s, 9H), 2.54 (s, 4H), 2.29 (d, J=7.6 Hz, 3H), 2.24 (dd, J=51.5, 12.0 Hz, 4H), 2.19 (d, J=16.3 Hz, 1H), 2.02 (d, J=14.4 Hz, 7H), 1.87 (d, J=10.0 Hz, 1H), 1.71 (s, 2H), 1.95-1.54 (m, 5H), 1.53 (dd, J=22.8, 11.0 Hz, 1H), 1.51 (d, J=8.1 Hz, 1H), 0.92 (t, J=6.6 Hz, 3H), 0.92 (t, J=6.6 Hz, 4H). [M+H]⁺=971.6.

### Example 292: 3-(4-(2-((S)-4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)-2-(methoxymethyl)piperazin-1-yl)ethyl)-2-fluorophenyl)piperidine-2,6-dione

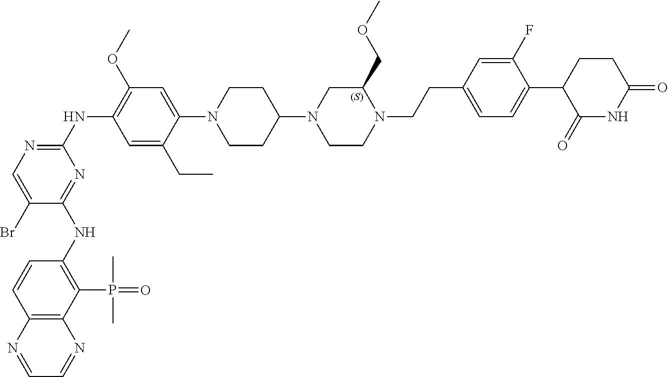

The titled compound was prepared in a manner similar to that in Example 291. 1H NMR (500 MHz, DMSO) δ 12.58 (s, 1H), 10.80 (s, 1H), 8.80 (d, J=6.9 Hz, 3H), 8.20 (s, 2H), 7.83 (d, J=9.0 Hz, 1H), 7.31 (s, 1H), 7.15 (s, 1H), 7.05-6.92 (m, 2H), 6.74 (s, 1H), 3.94 (d, J=12.5 Hz, 1H), 3.70 (s, 3H), 3.45 (d, J=4.5 Hz, 2H), 3.18 (s, 3H), 2.94 (d, J=10.6 Hz, 2H), 2.76 (d, J=22.4 Hz, 2H), 2.71-2.58 (m, 7H), 2.54 (s, 2H), 2.47 (s, 2H), 2.37 (s, 2H), 2.30 (s, 1H), 2.23 (s, 1H), 2.11 (dd, J=27.7, 14.4 Hz, 2H), 1.95 (d, J=14.4 Hz, 7H), 1.77 (d, J=10.2 Hz, 2H), 1.50 (d, J=3.1 Hz, 2H), 0.86 (t, J=7.0 Hz, 3H). [M+H]⁺=971.7.

### Example 293: 3-(4-(2-((R)-4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)-2-(methoxymethyl)piperazin-1-yl)ethyl)-2-fluorophenyl)piperidine-2,6-dione

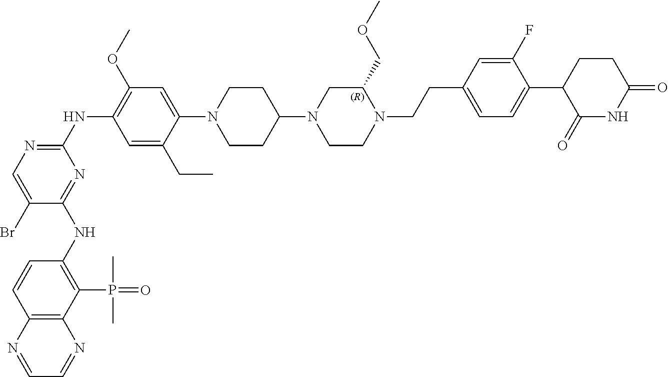

The titled compound was prepared in a manner similar to that in Example 291. 1H NMR (500 MHz, DMSO) δ 12.58 (s, 1H), 10.79 (s, 1H), 8.76 (d, J=22.3 Hz, 3H), 8.20 (s, 2H), 7.83 (s, 1H), 7.31 (s, 1H), 7.14 (d, J=15.7 Hz, 1H), 6.99 (d, J=19.4 Hz, 2H), 6.74 (s, 1H), 3.93 (d, J=17.4 Hz, 1H), 3.70 (s, 3H), 3.45 (d, J=14.3 Hz, 2H), 3.18 (s, 3H), 2.95 (d, J=10.6 Hz, 3H), 2.77 (d, J=16.6 Hz, 2H), 2.65 (dd, J=25.7, 10.1 Hz, 7H), 2.59-2.50 (m, 3H), 2.48 (s, 1H), 2.33 (d, J=41.4 Hz, 2H), 2.17-2.06 (m, 2H), 1.95 (d, J=14.4 Hz, 8H), 1.78 (d, J=11.5 Hz, 2H), 1.50 (d, J=12.1 Hz, 2H), 0.86 (t, J=6.9 Hz, 3H). [M+H]⁺=971.7.

### Example 298: 3-(4-(((1r,3r)-3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)cyclobutyl)amino)phenyl)piperidine-2,6-dione

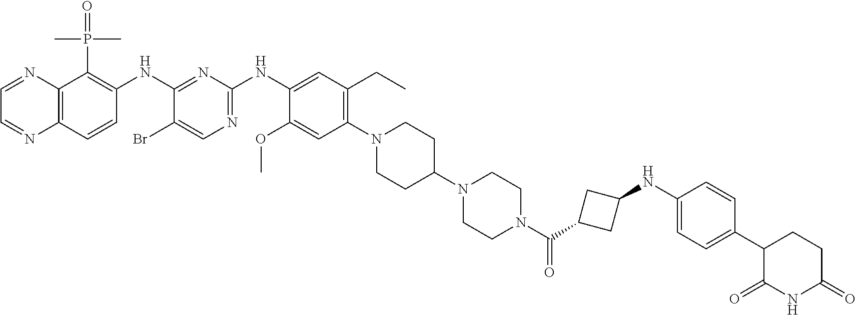 [SMILES: CCc1cc(Nc2ncc(Br)c(Nc3ccc4nccnc4c3P(C)(C)=O)n2)c(OC)cc1N1CCC(N2CCN(C(=O)[C@H]3C[C@H](Nc4ccc(C5CCC(=O)NC5=O)cc4)C3)CC2)CC1]

The titled compound was prepared in a manner similar to that in Example 208. 1H NMR (500 MHz, DMSO) δ 12.65 (s, 1H), 10.74 (s, 1H), 8.89-8.79 (m, 3H), 8.30-8.24 (m, 2H), 7.94-7.87 (m, 1H), 7.41-7.35 (m, 1H), 6.91 (d, J=8.5 Hz, 2H), 6.81 (s, 1H), 6.42 (d, J=8.6 Hz, 2H), 5.91 (d, J=5.9 Hz, 1H), 3.81-3.73 (m, 4H), 3.67-3.60 (m, 1H), 3.53-3.42 (m, 2H), 3.31-3.27 (m, 6H), 3.05-2.99 (m, 2H), 2.75-2.67 (m, 2H), 2.65-2.52 (m, 4H), 2.46-2.41 (m, 3H), 2.37-2.35 (m, 1H), 2.13-1.96 (m, 11H), 1.88-1.82 (m, 2H), 1.67-1.55 (m, 2H), 0.97-0.88 (m, 3H). [M+H]⁺=978.5.

### Example 299: 3-(4-(4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)piperidin-1-yl)phenyl)piperidine-2,6-dione

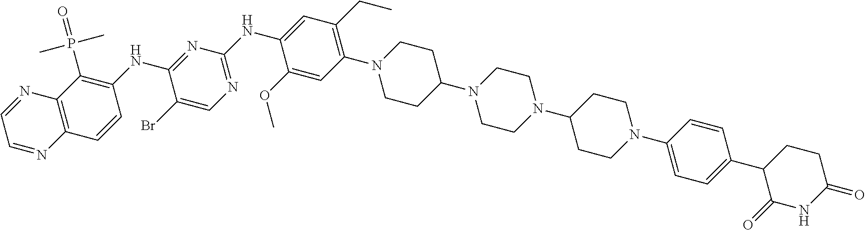 [SMILES: CCc1cc(Nc2ncc(Br)c(Nc3ccc4nccnc4c3P(C)(C)=O)n2)c(OC)cc1N1CCC(N2CCN(C3CCN(c4ccc(C5CCC(=O)NC5=O)cc4)CC3)CC2)CC1]

The titled compound was prepared in a manner similar to that in Example 200. 1H NMR (500 MHz, DMSO) δ 12.64 (s, 1H), 10.78 (s, 1H), 8.85 (t, J=14.2 Hz, 3H), 8.26 (t, J=14.3 Hz, 3H), 7.90 (d, J=8.0 Hz, 1H), 7.37 (s, 1H), 7.04 (d, J=8.6 Hz, 2H), 6.89 (d, J=8.6 Hz, 2H), 6.81 (s, 1H), 3.77 (s, 3H), 3.74-3.68 (m, 3H), 3.51 (s, 1H), 3.01 (d, J=11.7 Hz, 3H), 2.67 (dt, J=23.0,11.3 Hz, 7H), 2.54 (s, 3H), 2.46 (d, J=16.7 Hz, 3H), 2.29 (s, 2H), 2.13 (d, J=8.3 Hz, 2H), 2.02 (d, J=14.4 Hz, 6H), 1.85 (s, 4H), 1.60-1.47 (m, 4H), 1.24 (s, 1H), 0.93 (s, 3H). [M+H]⁺=964.4.

### Example 302: 3-(4-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)azetidin-1-yl)-2,6-difluorophenyl)piperidine-2,6-dione

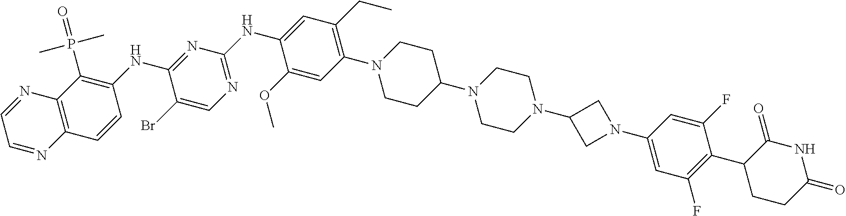 [SMILES: CCc1cc(Nc2ncc(Br)c(Nc3ccc4nccnc4c3P(C)(C)=O)n2)c(OC)cc1N1CCC(N2CCN(C3CN(c4cc(F)c(C5CCC(=O)NC5=O)c(F)c4)C3)CC2)CC1]

The titled compound was prepared in a manner similar to that in Example 200. ¹H NMR (500 MHz, DMSO) δ 12.64 (s, 1H), 10.86 (s, 1H), 8.86 (dd, J=8.1, 1.8 Hz, 2H), 8.84-8.78 (m, 1H), 8.27 (s, 2H), 8.13 (s, 1H), 7.90 (d, J=9.0 Hz, 1H), 7.38 (s, 1H), 6.81 (s, 1H), 6.13 (d, J=11.0 Hz, 2H), 4.03 (dd, J=12.3, 4.8 Hz, 1H), 3.91 (t, J=7.4 Hz, 2H), 3.77 (s, 3H), 3.67-3.60 (m, 2H), 3.30-3.23 (m, 2H), 3.05-2.98 (m, 2H), 2.84-2.66 (m, 4H), 2.66-2.54 (m, 5H), 2.39 (ddd, J=15.1, 12.2, 7.0 Hz, 7H), 2.11-1.98 (m, 8H), 1.97-1.84 (m, 3H), 1.65-1.54 (m, 2H), 0.97-0.90 (m, 3H). [M+H]⁺=972.5.

### Example 303: 3-(4-(4-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)piperidin-1-yl)-2,6-difluorophenyl)piperidine-2,6-dione

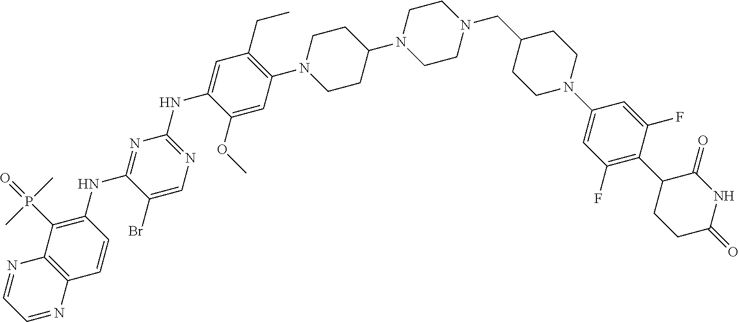 [SMILES: CCc1cc(Nc2ncc(Br)c(Nc3ccc4nccnc4c3P(C)(C)=O)n2)c(OC)cc1N1CCC(N2CCN(CC3CCN(c4cc(F)c(C5CCC(=O)NC5=O)c(F)c4)CC3)CC2)CC1]

The titled compound was prepared in a manner similar to that in Example 200. ¹H NMR (500 MHz, DMSO) δ 12.64 (s, 1H), 10.86 (s, 1H), 8.86 (dt, J=18.7, 9.3 Hz, 3H), 8.27 (s, 2H), 7.90 (d, J=8.5 Hz, 1H), 7.38 (s, 1H), 6.81 (s, 1H), 6.61 (d, J=12.9 Hz, 2H), 4.04 (dd, J=12.6, 5.0 Hz, 1H), 3.75 (d, J=17.2 Hz, 5H), 3.29 (s, 2H), 3.01 (d, J=10.9 Hz, 2H), 2.83-2.62 (m, 6H), 2.59-2.52 (m, 3H), 2.39 (m, 6H), 2.14 (d, J=6.8 Hz, 3H), 2.02 (d, J=14.4 Hz, 6H), 1.98-1.83 (m, 3H), 1.79-1.67 (m, 3H), 1.58 (dt, J=18.5, 4.8 Hz, 2H), 1.18-1.07 (m, 2H), 0.92 (t, J=7.1 Hz, 3H). [M/2+H]⁺=507.9.

### Example 306: 5-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)-2-(2,6-dioxopiperidin-3-yl)benzonitrile

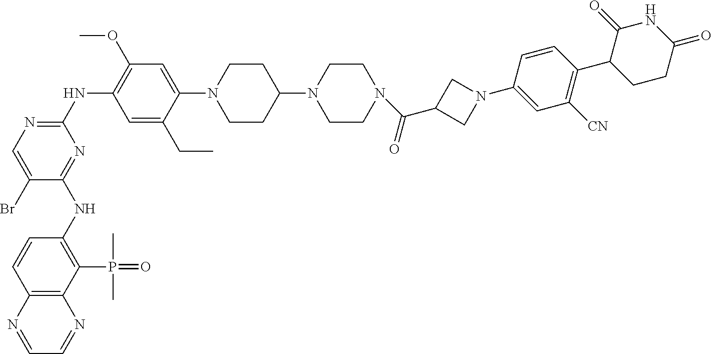 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc(C5CCC(=O)NC5=O)c(C#N)c4)C3)CC2)CC1]

The titled compound was prepared in a manner similar to that in Example 208. ¹H NMR (500 MHz, DMSO) δ 12.65 (s, 1H), 10.92 (s, 1H), 8.85 (dd, J=13.1, 11.5 Hz, 3H), 8.28 (d, J=8.2 Hz, 2H), 7.90 (d, J=8.6 Hz, 1H), 7.37 (s, 1H), 7.25 (d, J=8.6 Hz, 1H), 6.88 (t, J=7.4 Hz, 1H), 6.81 (s, 1H), 6.75 (dd, J=8.5, 2.4 Hz, 1H), 4.08 (t, J=7.8 Hz, 2H), 4.03-3.90 (m, 3H), 3.90-3.80 (m, 2H), 3.77 (s, 3H), 3.50 (s, 3H), 3.03 (d, J=10.8 Hz, 2H), 2.79 (dd, J=21.2, 9.2 Hz, 1H), 2.73-2.71 (m, 2H), 2.56 (d, J=3.9 Hz, 3H), 2.48-2.35 (m, 3H), 2.31-2.28 (m, 1H), 2.04-2.00 (m, 8H), 1.85 (d, J=10.3 Hz, 2H), 1.64-1.59 (m, 2H), 0.96-0.90 (m, 3H). [M+H]⁺=989.3.

### Example 307: 3-(3-(((1r,3r)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)cyclobutyl)(methyl)amino)phenyl)piperidine-2,6-dione

### Step 1: methyl (1r,3r)-3-((tert-butoxycarbonyl)amino)cyclobutane-1-carboxylate

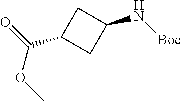 [SMILES: COC(=O)[C@H]1C[C@H](NC(=O)OC(C)(C)C)C1]

To a solution of methyl (1r,3r)-3-aminocyclobutane-1-carboxylate hydrochloride (1.5 g, 9.09 mmol) in dioxane (30 mL) and water (15 mL) was added Na₂CO₃ (2.89 g, 27.3 mmol) and Boc₂O (2.97 g, 13.6 mmol). The mixture was stirred at 20° C. for 13 hrs, the resulting mixture was extracted with EtOAc (70 mL×3). The combined organic phase was washed with brine (60 mL), dried over Na₂SO₄, filtered and concentrated in vacuum. The residue was purified by silica gel column (PE:EA=1:1). Methyl (1r,3r)-3-((tert-butoxycarbonyl)amino)cyclobutane-1-carboxylate (1.7 g, 81.7%) was obtained. [M+H]⁺=230.1.

### Step 2: methyl (1r,3r)-3-((tert-butoxycarbonyl)(methyl)amino)cyclobutane-1-carboxylate

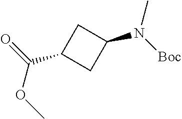 [SMILES: COC(=O)[C@H]1C[C@H](N(C)C(=O)OC(C)(C)C)C1]

To a solution of methyl (1r,3r)-3-((tert-butoxycarbonyl)amino)cyclobutane-1-carboxylate (1.5 g, 6.52 mmol) in DMF (20 mL) was added NaH (522 mg, 13.04 mmol, 60%) at 0° C. The mixture was stirred at 20° C. for 1 hr, then Mel (1.39 g, 9.78 mmol) was added, the reaction was stirred at r.t for 3 hrs and then quenched by sat. NH₄Cl solution (20 mL). The resulting mixture was extracted with EtOAc (40 mL×3). The combined organic phase was washed with brine (30 mL), dried over Na₂SO₄, filtered and concentrated in vacuum. The residue was purified by silica gel column (PE:EA=1:1). Methyl (1r,3r)-3-((tert-butoxycarbonyl)(methyl)amino)cyclobutane-1-carboxylate (1.4 g, 88%) was obtained. [M+H]⁺=244.2.

### Step 3: methyl (1r,3r)-3-(methylamino)cyclobutane-1-carboxylate hydrochloride

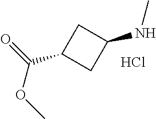

To a mixture of methyl (1r,3r)-3-((tert-butoxycarbonyl)(methyl)amino)cyclobutane-1-carboxylate (1.4 g, 5.76 mmol) and HCl in dioxane (20 mL, 6N) was stirred at 25° C. for 5 hr, then the resulting mixture was concentrated in vacuum. Methyl (1r,3r)-3-(methylamino)cyclobutane-1-carboxylate hydrochloride (700 mg, 84.4%) was obtained. [M+H]⁺=144.2.

### Step 4: methyl (1r,3r)-3-((3-(2,6-bis(benzyloxy)pyridin-3-yl)phenyl)(methyl)amino)cyclobutane-1-carboxylate

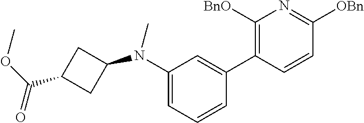 [SMILES: COC(=O)[C@H]1C[C@H](N(C)c2cccc(-c3ccc(OCc4ccccc4)nc3OCc3ccccc3)c2)C1]

Under the atmosphere of nitrogen, 2,6-bis(benzyloxy)-3-(3-bromophenyl)pyridine (1 g, 2.25 mmol), methyl (1r,3r)-3-(methylamino)cyclobutane-1-carboxylate hydrochloride (603 mg, 3.37 mmol) and Cs₂CO₃ (2.93 g, 9 mmol) were added to 1,4-dioxane (50 mL). After pumping nitrogen three times, Pd₂(dba)₃ (211 mg, 0.23 mmol) and Xantphos (266 mg, 0.46 mmol) were added to the mixture, and then nitrogen was pumped for three times again, then temperature was raised to reflux. After 15h, the reaction was cooled to room temperature, water (30 mL) was added, extracted with DCM (3×50 mL). The combined organic phases were washed with water and brine, dried and concentrated. The residue was purified with silica gel column (petroleum ether: ethyl acetate=3: 1) to obtain the product (500 mg, 43.7%). [M+H]⁺=509.2.

### Step 5: (1r,3r)-3-((3-(2,6-bis(benzyloxy)pyridin-3-yl)phenyl)(methyl)amino)cyclobutane-1-carboxylic acid

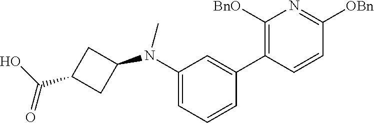 [SMILES: CN(c1cccc(-c2ccc(OCc3ccccc3)nc2OCc2ccccc2)c1)[C@H]1C[C@H](C(=O)O)C1]

To a solution of methyl (1r,3r)-3-((3-(2,6-bis(benzyloxy)pyridin-3-yl)phenyl)(methyl)amino)cyclobutane-1-carboxylate (500 mg, 0.98 mmol) in THF (10 mL) and H₂O (2 mL) was added lithium hydroxide hydrate (118 mg, 4.91 mmol) at 25° C. The resulting mixture was stirred at 25° C. for 12 h. The reaction was quenched with HCl (1 N) at 0° C. until pH=5 and extracted with DCM (2×40 mL). The combined organic layers were washed with brine, dried over anhydrous Na₂SO₄, and evaporated under vacuum to afford the crude product (480 mg, 99%), which was used for next step without further purification. [M+H]⁺=495.2.

### Step 6: ((1r,3r)-3-((3-(2,6-bis(benzyloxy)pyridin-3-yl)phenyl)(methyl)amino)cyclobutyl)methanol

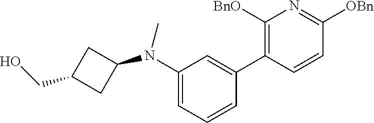 [SMILES: CN(c1cccc(-c2ccc(OCc3ccccc3)nc2OCc2ccccc2)c1)[C@H]1C[C@H](CO)C1]

To a solution of (1r,3r)-3-((3-(2,6-bis(benzyloxy)pyridin-3-yl)phenyl)(methyl)amino)cyclobutane-1-carboxylic acid (370 mg, 0.74 mmol) in THF (10 mL) was added BH3 in THF (1M) (1.48 mL, 1.48 mmol), the resulting mixture was stirred at 25° C. for 16 h. The reaction was quenched by addition CH₃OH (5 mL), the resulting solution was concentrated in vacuo. The residue was purified with silica gel column (petroleum ether: ethyl acetate=1: 1) to obtain the product (300 mg, 84.2%). [M+H]⁺=481.1.

### Step 7: 3-(3-(((1r,3r)-3-(hydroxymethyl)cyclobutyl)(methyl)amino)phenyl)piperidine-2,6-dione

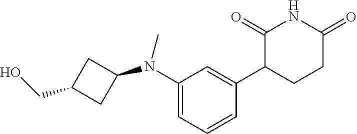 [SMILES: CN(c1cccc(C2CCC(=O)NC2=O)c1)[C@H]1C[C@H](CO)C1]

To a solution of ((1r,3r)-3-((3-(2,6-bis(benzyloxy)pyridin-3-yl)phenyl)(methyl)amino)cyclobutyl)methanol (300 mg, 0.62 mmol) in DCM (10 mL) and CH₃OH (3 mL) was added Pd/C (150 mg, 10%), the resulting mixture was stirred at 25° C. for 16 h under H₂ atmosphere. The catalyst was filtered off, the filtrate was concentrated in vacuo to obtain the product (160 mg, 85.1%). [M+H]⁺=303.2.

### Step 8: (1r,3r)-3-((3-(2,6-dioxopiperidin-3-yl)phenyl)(methyl)amino)cyclobutane-1-carbaldehyde

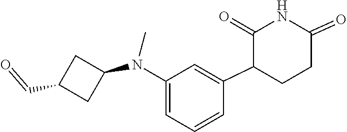 [SMILES: CN(c1cccc(C2CCC(=O)NC2=O)c1)[C@H]1C[C@H](C=O)C1]

To a solution of 3-(3-(((1r,3r)-3-(hydroxymethyl)cyclobutyl)(methyl)amino)phenyl)piperidine-2,6-dione (100 mg, 0.33 mmol) in DMSO (3 mL) was added IBX (139 mg, 0.5 mmol), water (5 mL) was added, extracted with DCM (3×20 mL). The combined organic phases were washed with sat. NaHCO₃ solution (10 mL×3) and brine (15 mL), dried and concentrated. The residue was purified with prep-TLC (pure ethylacetate) to obtain the product (30 mg, 30.3%). [M+H]⁺=301.2.

### Step 7: 3-(3-(((1r,3r)-3-((4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)methyl)cyclobutyl)(methyl)amino)phenyl)piperidine-2,6-dione

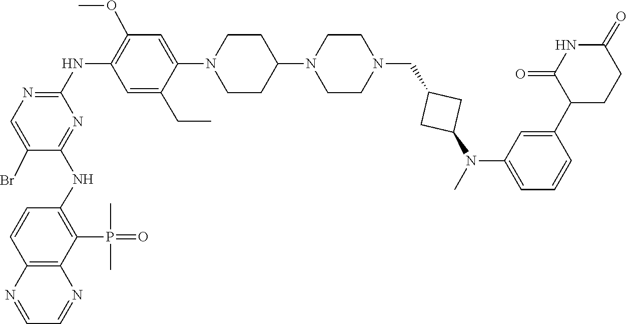 [SMILES: CCc1cc(Nc2ncc(Br)c(Nc3ccc4nccnc4c3P(C)(C)=O)n2)c(OC)cc1N1CCC(N2CCN(C[C@H]3C[C@H](N(C)c4cccc(C5CCC(=O)NC5=O)c4)C3)CC2)CC1]

The titled compound was prepared in a manner similar to that in Example 204 Step 6 from (1r,3r)-3-((3-(2,6-dioxopiperidin-3-yl)phenyl)(methyl)amino)cyclobutane-1-carbaldehyde and (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide. ¹H NMR (500 MHz, DMSO) δ 12.64 (s, 1H), 10.80 (s, 1H), 8.85 (dt, J=23.4, 11.7 Hz, 3H), 8.34-8.19 (m, 3H), 7.90 (d, J=9.2 Hz, 1H), 7.37 (s, 1H), 7.11 (t, J=7.9 Hz, 1H), 6.81 (s, 1H), 6.65 (t, J=7.9 Hz, 1H), 6.60 (s, 1H), 6.52 (t, J=6.1 Hz, 1H), 3.91-3.68 (m, 5H), 3.01 (d, J=10.7 Hz, 3H), 2.78 (s, 1H), 2.75 (s, 2H), 2.72-2.69 (m, 2H), 2.65-2.59 (m, 2H), 2.54 (s, 1H), 2.45 (d, J=4.8 Hz, 2H), 2.41-2.23 (m, 8H), 2.19-2.09 (m, 3H), 2.08-1.95 (m, 8H), 1.85 (d, J=11.0 Hz, 2H), 1.64-1.60 (m, 4H), 0.95-0.90 (m, 3H). [M+H]⁺=978.4.

### Example 308: 3-(5-(4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)piperidin-1-yl)-2H-indazol-2-yl)piperidine-2,6-dione

### Step 1: 3-(5-bromo-2H-indazol-2-yl)-1-(4-methoxybenzyl)piperidine-2,6-dione

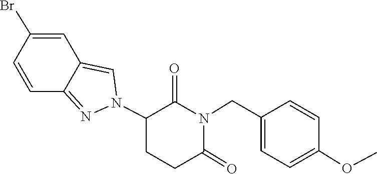 [SMILES: COc1ccc(CN2C(=O)CCC(n3cc4cc(Br)ccc4n3)C2=O)cc1]

To a solution of 1-(4-methoxybenzyl)-2,6-dioxopiperidin-3-yl trifluoromethanesulfonate (12.5 g, 32.9 mmol) and 5-bromo-2H-indazole (4.3 g, 21.9 mmol) in THF (100 mL) was added KOtBu in THF (1 M, 32.9 mL) at 0° C. Then the mixture was stirred at 20° C. for 48 hrs. Upon cooling with ice, the reaction was quenched by water (50.0 mL) and the resulting mixture was extracted with EtOAc (1000.0 mL). The combined organic phase was washed with brine (40.0 mL), dried over Na₂SO₄, filtered and concentrated in vacuum. The residue was purified by silica gel column (PE:EA=2:1). 3-(5-bromo-2H-indazol-2-yl)-1-(4-methoxybenzyl)piperidine-2,6-dione (2 g, 21.3%) was obtained. [M+H]⁺=428.2.

### Step 2: Methyl 1-(2-(1-(4-methoxybenzyl)-2,6-dioxopiperidin-3-yl)-2H-indazol-5-yl)piperidine-4-carboxylate

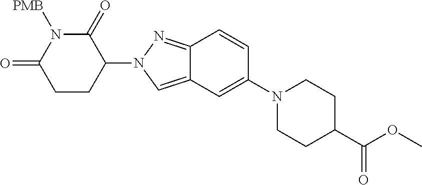

To a mixture of 3-(5-bromo-2H-indazol-2-yl)-1-(4-methoxybenzyl)piperidine-2,6-dione (1 g, 2.34 mmol), methyl piperidine-4-carboxylate (502 mg, 3.51 mmol), Ruphos Pd G3 (198 mg, 0.23 mmol), Cs2CO3 (1.53 g, 4.68 mmol) in toluene (15 mL) was stirred at 100° C. for 15 hrs. After cooling to r.t, the solid was filtered off, the filtrate was concentrated in vacuo. The residue was purified by silica gel column (PE:EA=1:1) to afford product (520 mg, 45.3%). [M+H]⁺=491.3.

### Step 3: 1-(2-(1-(4-methoxybenzyl)-2,6-dioxopiperidin-3-yl)-2H-indazol-5-yl)piperidine-4-carboxylic acid

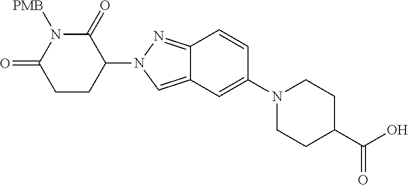

To a solution of methyl 1-(2-(1-(4-methoxybenzyl)-2,6-dioxopiperidin-3-yl)-2H-indazol-5-yl)piperidine-4-carboxylate (520 mg, 1.06 mmol) in THF (12 mL) and water (6 mL) was added LiOH (127 mg, 5.30 mmol). Then the mixture was stirred at 20° C. for 16 hrs. HCl (1N) was added to PH=5-6, the resulting mixture was extracted with EtOAc (20 mL×3). The combined organic phase was washed with brine (10 mL), dried over Na₂SO₄, filtered and concentrated in vacuum. 1-(2-(1-(4-methoxybenzyl)-2,6-dioxopiperidin-3-yl)-2H-indazol-5-yl)piperidine-4-carboxylic acid (490 mg, 97%) was obtained. [M+H]⁺=477.2.

### Step 4: 1-(2-(2,6-dioxopiperidin-3-yl)-2H-indazol-5-yl)piperidine-4-carboxylic acid

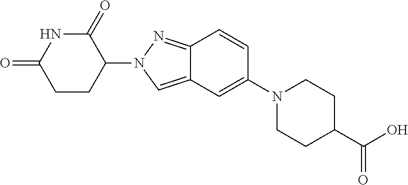 [SMILES: O=C1CCC(n2cc3cc(N4CCC(C(=O)O)CC4)ccc3n2)C(=O)N1]

To a mixture of 1-(2-(1-(4-methoxybenzyl)-2,6-dioxopiperidin-3-yl)-2H-indazol-5-yl)piperidine-4-carboxylic acid (490 mg, 1.03 mmol) in toluene (6 mL) was added MsOH (2 mL) was stirred at 80° C. for 15 hrs. After cooling to r.t, the reaction mixture was concentrated in vacuo. The residue was purified by reversed phase column (CH3CN:H2O (FA)=30:100) to afford the desired product (100 mg, 27.3%). [M+H]⁺=357.1.

### Step 5: 3-(5-(4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)piperidin-1-yl)-2H-indazol-2-yl)piperidine-2,6-dione

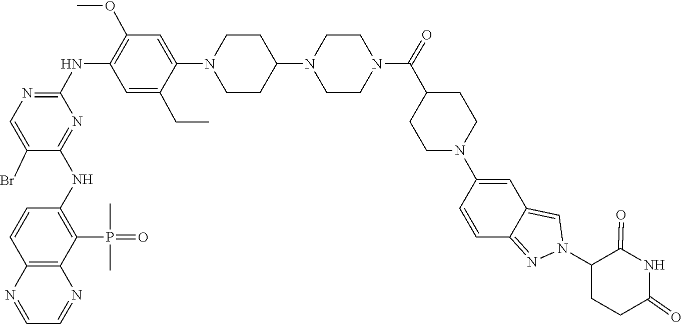 [SMILES: CCc1cc(Nc2ncc(Br)c(Nc3ccc4nccnc4c3P(C)(C)=O)n2)c(OC)cc1N1CCC(N2CCN(C(=O)C3CCN(c4ccc5nn(C6CCC(=O)NC6=O)cc5c4)CC3)CC2)CC1]

The titled compound (5 mg, 16%) was prepared in a manner similar to that in Example 208 step 8 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and 1-(2-(2,6-dioxopiperidin-3-yl)-2H-indazol-5-yl)piperidine-4-carboxylic acid. ¹H NMR (500 MHz, DMSO) δ 12.58 (s, 1H), 11.06 (s, 1H), 8.78 (t, J=16.0 Hz, 3H), 8.21 (s, 2H), 8.12 (s, 1H), 7.84 (d, J=8.7 Hz, 1H), 7.40 (d, J=9.4 Hz, 1H), 7.31 (s, 1H), 7.09 (d, J=9.7 Hz, 1H), 6.85 (s, 1H), 6.75 (s, 1H), 5.59-5.44 (m, 1H), 3.73 (d, J=27.0 Hz, 3H), 3.58-3.38 (m, 7H), 3.10 (d, J=4.9 Hz, 1H), 2.96 (d, J=10.3 Hz, 3H), 2.84-2.54 (m, 9H), 2.51 (s, 2H), 2.31-2.27 (m, 2H), 1.96-1.92 (m, 6H), 1.80 (d, J=10.9 Hz, 2H), 1.68 (s, 4H), 1.59-1.43 (m, 2H), 0.90-0.84 (m, 3H). [M+H]⁺=1032.4.

### Example 309: 3-(5-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2H-indazol-2-yl)piperidine-2,6-dione

### Step 1: 3-(5-bromo-2H-indazol-2-yl)-1-(4-methoxybenzyl)piperidine-2,6-dione

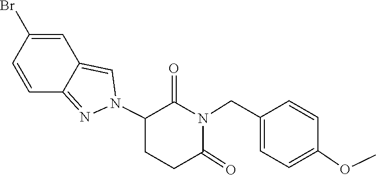 [SMILES: COc1ccc(CN2C(=O)CCC(n3cc4cc(Br)ccc4n3)C2=O)cc1]

To a solution of 1-(4-methoxybenzyl)-2,6-dioxopiperidin-3-yl trifluoromethanesulfonate (12.5 g, 32.9 mmol) and 5-bromo-2H-indazole (4.3 g, 21.9 mmol) in THF (100 mL) was added KOtBu in THF (1 M, 32.9 mL) at 0° C. Then the mixture was stirred at 20° C. for 48 hrs. Upon cooling with ice, the reaction was quenched by water (50.0 mL) and the resulting mixture was extracted with EtOAc (100.0 mL). The combined organic phase was washed with brine (40.0 mL), dried over Na₂SO₄, filtered and concentrated in vacuum. The residue was purified by silica gel column (PE:EA=2:1). 3-(5-bromo-2H-indazol-2-yl)-1-(4-methoxybenzyl)piperidine-2,6-dione (2 g, 21.3%) was obtained. [M+H]⁺=428.2.

### Step 2: 3-(5-bromo-2H-indazol-2-yl)piperidine-2,6-dione

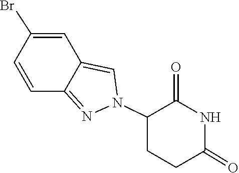 [SMILES: O=C1CCC(n2cc3cc(Br)ccc3n2)C(=O)N1]

To a mixture of 3-(5-bromo-2H-indazol-2-yl)-1-(4-methoxybenzyl)piperidine-2,6-dione (1 g, 2.33 mmol) in toluene (5 mL) was added MsOH (5 mL). Then the mixture was stirred at 100° C. for 16 hrs. Upon cooling with ice, the reaction was concentrated in vacuo. The residue was purified by silica gel column (PE:EA=1:2). 3-(5-bromo-2H-indazol-2-yl)piperidine-2,6-dione (520 mg, 72.5%) was obtained. [M+H]⁺=308.

### Step 3: (E)-3-(5-(2-ethoxyvinyl)-2H-indazol-2-yl)piperidine-2,6-dione

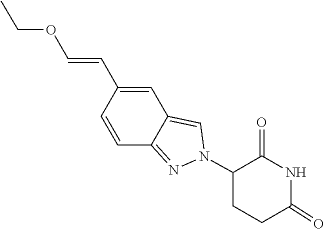 [SMILES: CCO/C=C/c1ccc2nn(C3CCC(=O)NC3=O)cc2c1]

To a mixture of 3-(5-bromo-2H-indazol-2-yl)piperidine-2,6-dione (520 mg, 1.69 mmol), (E)-2-(2-ethoxyvinyl)-4,4,5,5-tetramethyl-1,3,2-dioxaborolane (436 mg, 2.2 mmol), Pd(dppf)Cl₂ (124 mg, 0.17 mmol), CsF (514 mg, 3.38 mmol) in DMF (10 mL) and H₂O (2 mL) was stirred at 100° C. under N2 atmosphere for 1 hr. After cooling to r.t, the reaction mixture was extracted with EtOAc (20.0 mL×3). The combined organic phase was washed with brine (30.0 mL), dried over Na₂SO₄, filtered and concentrated in vacuum. The residue was purified by silica gel column (PE:EA=1:1). (E)-3-(5-(2-ethoxyvinyl)-2H-indazol-2-yl)piperidine-2,6-dione (460 mg, 91%) was obtained. [M+H]⁺=300.1.

### Step 4: 2-(2-(2,6-dioxopiperidin-3-yl)-2H-indazol-5-yl)acetaldehyde

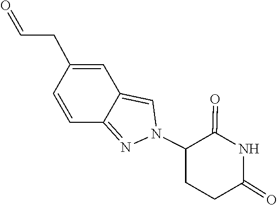 [SMILES: O=CCc1ccc2nn(C3CCC(=O)NC3=O)cc2c1]

To a solution of (E)-3-(5-(2-ethoxyvinyl)-2H-indazol-2-yl)piperidine-2,6-dione (460 mg, 1.54 mmol) in FA (5 mL) was stirred at 30° C. for 2 hrs. The reaction solution was concentrated in vacuum. 2-(2-(2,6-dioxopiperidin-3-yl)-2H-indazol-5-yl)acetaldehyde (400 mg, 96%) was obtained. [M+H]⁺=272.1.

### Step 5: 3-(5-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2H-indazol-2-yl)piperidine-2,6-dione

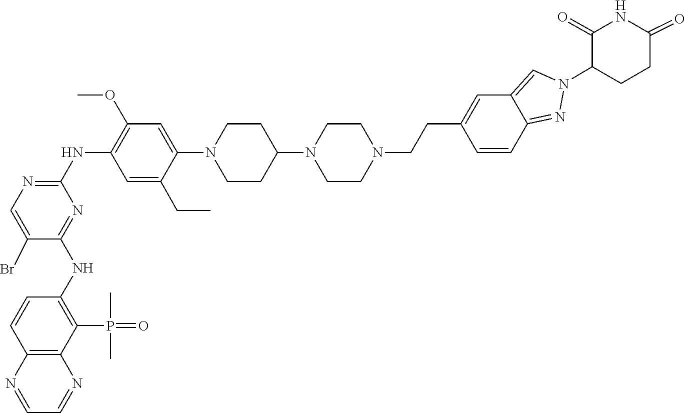 [SMILES: CCc1cc(Nc2ncc(Br)c(Nc3ccc4nccnc4c3P(C)(C)=O)n2)c(OC)cc1N1CCC(N2CCN(CCc3ccc4nn(C5CCC(=O)NC5=O)cc4c3)CC2)CC1]

The titled compound (12 mg, 23%) was prepared in a manner similar to that in Example 204 step 6 from 2-(2-(2,6-dioxopiperidin-3-yl)-2H-indazol-5-yl)acetaldehyde and (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide. ¹H NMR (500 MHz, DMSO) δ =12.68 (d, J=34.8, 1H), 11.16 (s, 1H), 8.86 (dt, J=18.9, 9.4, 3H), 8.33 (d, J=14.0, 1H), 8.27 (s, 2H), 7.90 (d, J=8.8, 1H), 7.61-7.43 (m, 2H), 7.38 (s, 1H), 7.16 (d, J=10.1, 1H), 6.96-6.65 (m, 1H), 5.68 (dd, J=11.4, 5.1, 1H), 3.84-3.69 (m, 3H), 3.01 (d, J=11.1, 2H), 2.92-2.63 (m, 8H), 2.63-2.52 (m, 7H), 2.47 (d, J=7.3, 3H), 2.35-2.30 (m, 3H), 2.02 (d, J=14.4, 6H), 1.85-1.81 (m, 2H), 1.69-1.43 (m, 2H), 0.93-0.87 (m, 3H). [M+H]⁺=949.3.

### Example 311: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)-7-azaspiro[3.5]nonan-7-yl)phenyl)piperidine-2,6-dione

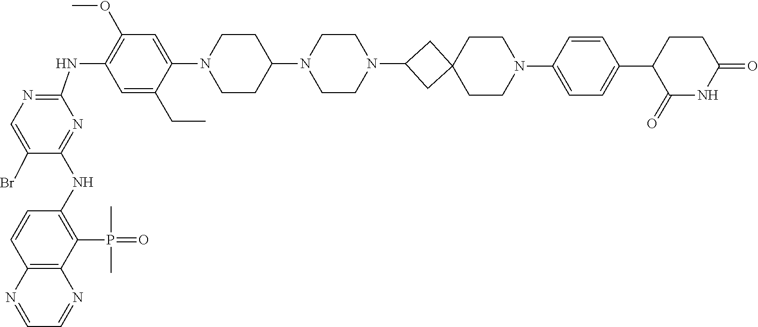 [SMILES: CCc1cc(Nc2ncc(Br)c(Nc3ccc4nccnc4c3P(C)(C)=O)n2)c(OC)cc1N1CCC(N2CCN(C3CC4(CCN(c5ccc(C6CCC(=O)NC6=O)cc5)CC4)C3)CC2)CC1]

The titled compound was prepared in a manner similar to that in Example 200. ¹H NMR (500 MHz, DMSO) δ 12.64 (s, 1H), 10.76 (s, 1H), 8.85 (dt, J=20.1, 10.0 Hz, 3H), 8.27 (s, 2H), 7.90 (d, J=9.3 Hz, 1H), 7.38 (s, 1H), 7.02 (d, J=8.6 Hz, 2H), 6.88 (d, J=8.7 Hz, 2H), 6.81 (s, 1H), 3.77 (s, 3H), 3.71 (dd, J=10.9, 4.9 Hz, 1H), 3.41 (s, 2H), 3.10 (s, 2H), 3.01 (d, J=6.5 Hz, 4H), 2.78-2.57 (m, 5H), 2.54-2.52 (m, 1H), 2.49-2.41 (m, 3H), 2.32 (t, J=18.1 Hz, 4H), 2.21-2.07 (m, 2H), 2.06 (s, 3H), 2.04-2.00 (m, 1H), 1.99 (s, 3H), 1.96-1.86 (m, 2H), 1.85 (d, J=10.9 Hz, 2H), 1.67-1.46 (m, 8H), 0.92 (t, J=7.0 Hz, 3H). [M+H]⁺=1004.4.

### Example 312: 3-(4-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

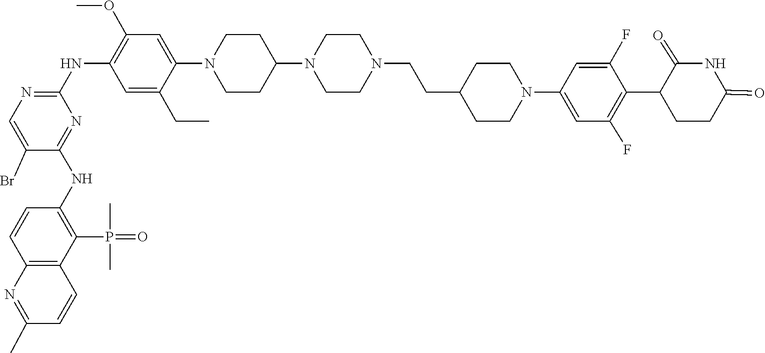 [SMILES: CCc1cc(Nc2ncc(Br)c(Nc3ccc4nc(C)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1]

The titled compound was prepared in a manner similar to that in Example 200. ¹H NMR (500 MHz, DMSO) δ 11.76 (s, 1H), 10.86 (s, 1H), 8.56 (d, J=9.0 Hz, 1H), 8.30 (s, 1H), 8.20 (d, J=14.2 Hz, 1H), 7.97 (s, 1H), 7.87 (d, J=9.1 Hz, 1H), 7.48-7.28 (m, 2H), 6.73 (s, 1H), 6.60 (d, J=12.8 Hz, 2H), 4.04 (dd, J=12.6, 5.1 Hz, 1H), 3.76-3.72 (m, 6H), 2.93 (d, J=11.7 Hz, 2H), 2.83-2.68 (m, 3H), 2.66 (d, J=15.0 Hz, 6H), 2.54-2.51 (m, 2H), 2.42-2.19 (m, 9H), 2.07 (t, J=12.7 Hz, 1H), 1.98 (d, J=13.3 Hz, 8H), 1.83 (d, J=10.8 Hz, 2H), 1.71 (d, J=11.0 Hz, 2H), 1.61-1.44 (m, 3H), 1.38 (d, J=6.7 Hz, 2H), 1.23-1.19 (m, 2H), 0.80-0.76 (m, 3H). [M+H]⁺=1041.4.

### Example 313: 3-(4-(4-(2-(4-(1-(4-((5-bromo-4-((5-(dimethylphosphoryl)-2-isopropylquinolin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

### Step 1: 2-chloroquinolin-6-amine

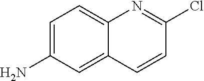 [SMILES: Nc1ccc2nc(Cl)ccc2c1]

To a solution of 2-chloro-6-nitroquinoline (5 g, 24.03 mmol) in EtOH (60 mL) and water (25 mL) was added Fe (powder, 6.73 g, 120 mmol) and NH4Cl (6.36 g, 120 mmol). The mixture was stirred at 70° C. for 3 hrs. After cooling to r.t, the solid was filtered off, the filtrate was extracted with DCM (70 mL×3). The combined organic phase was washed with brine (60 mL), dried over Na₂SO₄, filtered and concentrated in vacuum. 2-chloroquinolin-6-amine (4 g, 93%) was obtained. [M+H]⁺=179.2.

### Step 2: 2-chloro-5-iodoquinolin-6-amine

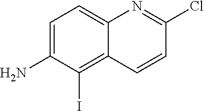 [SMILES: Nc1ccc2nc(Cl)ccc2c1I]

To a solution of 2-chloroquinolin-6-amine (4 g, 22.3 mmol) in HOAc (20 mL) was added ICl (4.24 g, 33.45 mmol). The mixture was stirred at 20° C. for 3 hrs and then sat. Na2CO3 solution was added to PH=8-9, the resulting mixture was extracted with EtOAc (70 mL×3). The combined organic phase was washed with brine (60 mL), dried over Na₂SO₄, filtered and concentrated in vacuum. 5-iodo-2-(trifluoromethyl)quinolin-6-amine (6.7 g, 88.6%) was obtained. [M+H]⁺=304.9.

### Step 3: (6-amino-2-chloroquinolin-5-yl)dimethylphosphine oxide

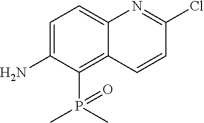 [SMILES: CP(C)(=O)c1c(N)ccc2nc(Cl)ccc12]

To a mixture of 2-chloro-5-iodoquinolin-6-amine (6.5 g, 21.3 mmol), dimethyl phosphine oxide (2.43 g, 32 mmol), Pd(OAc)2 (477 mg, 2.13 mmol), Xantphos (2.46 g, 4.26 mmol), K3PO4 (9.03 g, 42.6 mmol) in dioxane (110 mL) was stirred at 100° C. under N2 atmosphere for 18 hrs. After cooling to r.t, the reaction mixture was filtered, the filtrate was concentrated in vacuo. The residue was purified by silica gel column (DCM:CH3OH=15:1). (6-amino-2-chloroquinolin-5-yl)dimethylphosphine oxide (5.1 g, 94%) was obtained. [M+H]⁺=255.

### Step 4: (6-amino-2-(prop-1-en-2-yl)quinolin-5-yl)dimethylphosphine oxide

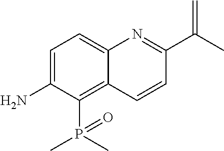 [SMILES: C=C(C)c1ccc2c(P(C)(C)=O)c(N)ccc2n1]

To a mixture of (6-amino-2-chloroquinolin-5-yl)dimethylphosphine oxide (1.1 g, 4.31 mmol), 4,4,5,5-tetramethyl-2-(prop-1-en-2-yl)-1,3,2-dioxaborolane (1.09 g, 6.47 mmol), Pd(dppf)Cl₂ (314 mg, 0.43 mmol), Na2CO3 (914 mg, 8.62 mmol) in dioxane (10 mL) and H₂O (2 mL) was stirred at 100° C. under N2 atmosphere for 3 hrs. After cooling to r.t, the reaction mixture was extracted with EtOAc (20.0 mL×3). The combined organic phase was washed with brine (30.0 mL), dried over Na₂SO₄, filtered and concentrated in vacuum. The residue was purified by silica gel column (DCM:CH3OH=15:1). (6-amino-2-(prop-1-en-2-yl)quinolin-5-yl)dimethylphosphine oxide (850 mg, 75.9%) was obtained. [M+H]⁺=261.1.

### Step 5: (6-amino-2-isopropylquinolin-5-yl)dimethylphosphine oxide

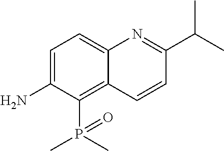 [SMILES: CC(C)c1ccc2c(P(C)(C)=O)c(N)ccc2n1]

To a solution of (6-amino-2-(prop-1-en-2-yl)quinolin-5-yl)dimethylphosphine oxide (850 mg, 3.27 mmol) in CH3OH (10 mL) was added Pd/C (300 mg, 10%) under H2 atmosphere. The reaction mixture was concentrated in vacuum. (6-amino-2-isopropylquinolin-5-yl)dimethylphosphine oxide (800 mg, 93%) was obtained. [M+H]⁺=263.1.

### Step 6: (6-((5-bromo-2-chloropyrimidin-4-yl)amino)-2-isopropylquinolin-5-yl)dimethylphosphine oxide

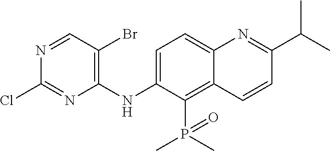 [SMILES: CC(C)c1ccc2c(P(C)(C)=O)c(Nc3nc(Cl)ncc3Br)ccc2n1]

The titled compound (610 mg, 51%) was prepared in a manner similar to that in Example 208 Step 6 from (6-amino-2-isopropylquinolin-5-yl)dimethylphosphine oxide and 5-bromo-2,4-dichloropyrimidine. [M+H]⁺=453.2.

### Step 7: (6-((5-bromo-2-((5-ethyl-2-methoxy-4-(4-(piperazin-1-yl)piperidin-1-yl)phenyl)amino)pyrimidin-4-yl)amino)-2-isopropylquinolin-5-yl)dimethylphosphine oxide

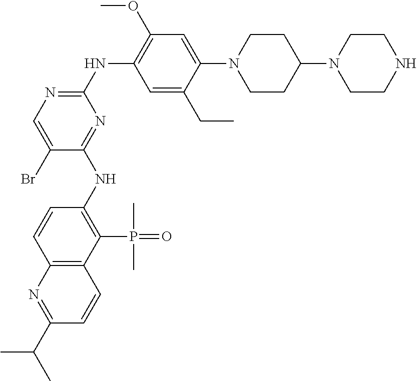 [SMILES: CCc1cc(Nc2ncc(Br)c(Nc3ccc4nc(C(C)C)ccc4c3P(C)(C)=O)n2)c(OC)cc1N1CCC(N2CCNCC2)CC1]

The titled compound (210 mg, 34%) was prepared in a manner similar to that in Example 208 Step 7 from (6-((5-bromo-2-chloropyrimidin-4-yl)amino)-2-isopropylquinolin-5-yl)dimethylphosphine oxide and tert-butyl 4-(1-(4-amino-2-ethyl-5-methoxyphenyl)piperidin-4-yl)piperazine-1-carboxylate. [M+H]⁺=735.3.

### Step 8: 3-(4-(4-(2-(4-(1-(4-((5-bromo-4-((5-(dimethylphosphoryl)-2-isopropylquinolin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

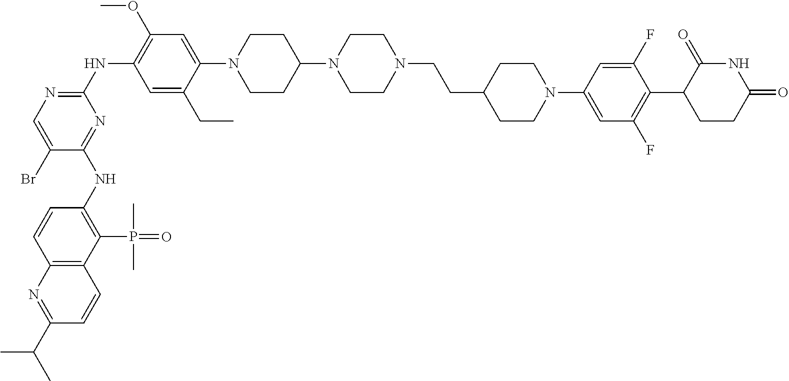 [SMILES: CCc1cc(Nc2ncc(Br)c(Nc3ccc4nc(C(C)C)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1]

The titled compound (12 mg, 23%) was prepared in a manner similar to that in Example 204 Step 6 from (6-((5-bromo-2-((5-ethyl-2-methoxy-4-(4-(piperazin-1-yl)piperidin-1-yl)phenyl)amino)pyrimidin-4-yl)amino)-2-isopropylquinolin-5-yl)dimethylphosphine oxide and 2-(1-(4-(2,6-dioxopiperidin-3-yl)-3,5-difluorophenyl)piperidin-4-yl)acetaldehyde. ¹H NMR (500 MHz, DMSO) δ 11.82 (s, 1H), 10.86 (s, 1H), 8.54 (d, J=8.8 Hz, 1H), 8.23 (d, J=26.8 Hz, 3H), 8.03 (s, 1H), 7.86 (d, J=9.4 Hz, 1H), 7.46 (d, J=9.0 Hz, 1H), 7.28 (s, 1H), 6.76 (s, 1H), 6.60 (d, J=12.9 Hz, 3H), 4.04 (dd, J=12.6, 4.9 Hz, 1H), 3.85-3.66 (m, 6H), 3.23-3.18 (m, 2H), 2.95 (d, J=10.7 Hz, 3H), 2.84-2.63 (m, 6H), 2.32-2.27 (m, 8H), 2.12-2.06 (m, 1H), 1.98 (s, 3H), 1.97 (s, 3H), 1.84 (d, J=11.7 Hz, 2H), 1.78-1.66 (m, 2H), 1.55-1.51 (m, 3H), 1.39 (s, 2H), 1.32 (s, 3H), 1.30 (s, 3H), 1.23-1.05 (m, 3H), 0.82-0.75 (m, 3H). [M+H]⁺=1069.4.

### Example 314: 3-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

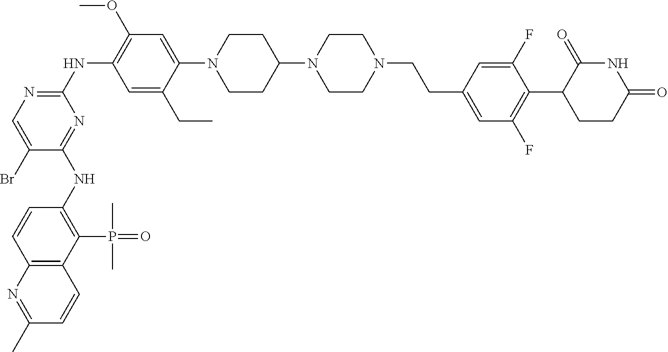 [SMILES: CCc1cc(Nc2ncc(Br)c(Nc3ccc4nc(C)ccc4c3P(C)(C)=O)n2)c(OC)cc1N1CCC(N2CCN(CCc3cc(F)c(C4CCC(=O)NC4=O)c(F)c3)CC2)CC1]

The titled compound was prepared in a manner similar to that in Example 200. ¹H NMR (500 MHz, DMSO) δ 11.76 (s, 1H), 10.95 (s, 1H), 8.56 (d, J=8.9 Hz, 1H), 8.29 (s, 1H), 8.20 (d, J=10.9 Hz, 1H), 7.97 (s, 1H), 7.87 (d, J=9.2 Hz, 1H), 7.48-7.25 (m, 2H), 7.03 (d, J=10.1 Hz, 2H), 6.73 (s, 1H), 4.20 (dd, J=12.7, 5.0 Hz, 1H), 3.75 (s, 3H), 2.93 (d, J=10.7 Hz, 2H), 2.87-2.70 (m, 3H), 2.67-2.60 (m, 6H), 2.56-2.52 (m, 6H), 2.46-2.39 (m, 4H), 2.29 (s, 4H), 2.16-2.10 (m, 1H), 1.99, (s, 3H), 1.97 (s, 3H), 1.86-1.81 (m, 2H), 1.57-1.52 (m, 2H), 0.82-0.75 (m, 3H). [M+H]⁺=958.3.

### Example 315: 3-(5-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)-2H-indazol-2-yl)piperidine-2,6-dione

### Step 1: tert-butyl 1-(2-(1-(4-methoxybenzyl)-2,6-dioxopiperidin-3-yl)-2H-indazol-5-yl)azetidine-3-carboxylate

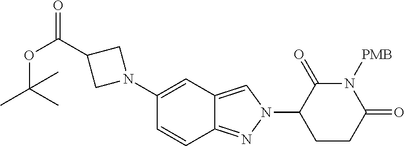

The titled compound (310 mg, 45%) was prepared in a manner similar to that in Example 308 Step 2 from 3-(5-bromo-2H-indazol-2-yl)-1-(4-methoxybenzyl)piperidine-2,6-dione and HCl salt of tert-butyl azetidine-3-carboxylate. [M+H]⁺=505.3.

### Step 2: 1-(2-(2,6-dioxopiperidin-3-yl)-2H-indazol-5-yl)azetidine-3-carboxylic acid

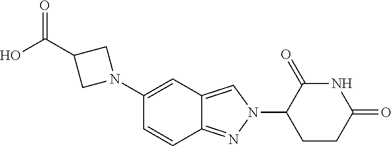 [SMILES: O=C1CCC(n2cc3cc(N4CC(C(=O)O)C4)ccc3n2)C(=O)N1]

To a mixture of tert-butyl 1-(2-(1-(4-methoxybenzyl)-2,6-dioxopiperidin-3-yl)-2H-indazol-5-yl)azetidine-3-carboxylate (410 mg, 0.81 mmol) in toluene (6 mL) was added MsOH (2 mL) was stirred at 80° C. for 15 hrs. After cooling to r.t, the reaction mixture was concentrated in vacuo. The residue was purified by reversed phase column (CH₃CN:H₂O(FA)=30:100). 1-(2-(2,6-dioxopiperidin-3-yl)-2H-indazol-5-yl)azetidine-3-carboxylic acid (90 mg, 34%) was obtained. [M+H]⁺=329.2.

### Step 3: 3-(5-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)azetidin-1-yl)-2H-indazol-2-yl)piperidine-2,6-dione

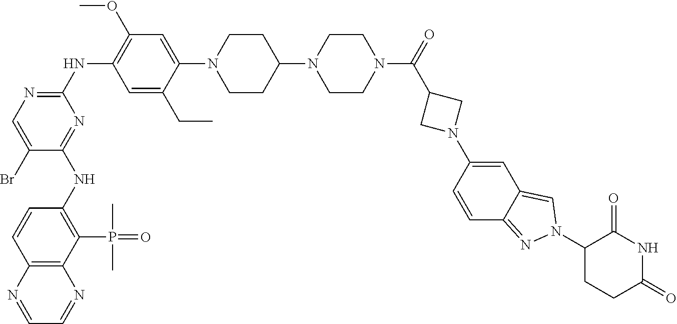 [SMILES: CCc1cc(Nc2ncc(Br)c(Nc3ccc4nccnc4c3P(C)(C)=O)n2)c(OC)cc1N1CCC(N2CCN(C(=O)C3CN(c4ccc5nn(C6CCC(=O)NC6=O)cc5c4)C3)CC2)CC1]

The titled compound (12 mg, 14%) was prepared in a manner similar to that in Example 208 Step 8 from 1-(2-(2,6-dioxopiperidin-3-yl)-2H-indazol-5-yl)azetidine-3-carboxylic acid and (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide. ¹H NMR (500 MHz, DMSO) δ 12.58 (s, 1H), 11.06 (s, 1H), 8.79 (dt, J=20.8, 10.4 Hz, 3H), 8.19 (d, J=14.1 Hz, 2H), 8.05 (s, 1H), 7.84 (d, J=9.0 Hz, 1H), 7.42 (d, J=9.2 Hz, 1H), 7.31 (s, 1H), 6.75 (s, 1H), 6.65 (dd, J=9.2, 2.0 Hz, 1H), 6.44 (d, J=35.7 Hz, 1H), 5.54 (dd, J=11.5, 5.2 Hz, 1H), 3.96 (t, J=7.3 Hz, 2H), 3.78 (dt, J=14.3, 6.7 Hz, 3H), 3.71 (s, 3H), 3.43 (s, 2H), 3.29 (s, 2H), 2.96 (d, J=11.1 Hz, 3H), 2.82-2.74 (m, 1H), 2.70-2.54 (m, 5H), 2.52-2.46 (m, 2H), 2.37-2.15 (m, 4H), 1.97 (s, 3H), 1.94 (s, 3H), 1.79 (d, J=12.0 Hz, 2H), 1.53-1.49 (m, 2H), 0.92-0.83 (m, 3H). [M+H]⁺=1004.3.

### Example 316: 3-(4-(2-(4-(1-(4-((5-bromo-4-((5-(dimethylphosphoryl)-2-isopropylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

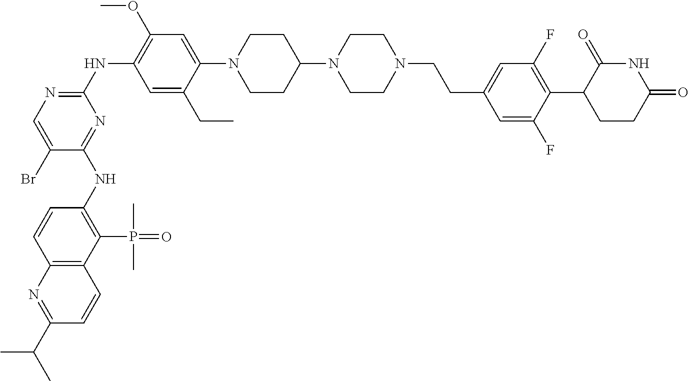 [SMILES: CCc1cc(Nc2ncc(Br)c(Nc3ccc4nc(C(C)C)ccc4c3P(C)(C)=O)n2)c(OC)cc1N1CCC(N2CCN(CCc3cc(F)c(C4CCC(=O)NC4=O)c(F)c3)CC2)CC1]

The titled compound was prepared in a manner similar to that in Example 208. ¹H NMR (500 MHz, DMSO) δ 11.81 (s, 1H), 10.95 (s, 1H), 8.55 (d, J=9.0 Hz, 1H), 8.22 (d, J=16.9 Hz, 2H), 8.03 (s, 1H), 7.86 (d, J=9.2 Hz, 1H), 7.46 (d, J=9.0 Hz, 1H), 7.29 (s, 1H), 7.03 (d, J=10.1 Hz, 2H), 6.76 (s, 1H), 4.38-3.91 (m, 1H), 3.76 (s, 3H), 3.29-3.27 (m, 2H), 3.20 (dt, J=13.7, 6.9 Hz, 2H), 2.98-2.93 (m, 2H), 2.87-2.73 (m, 3H), 2.72-2.63 (m, 3H), 2.58-2.52 (m, 5H), 2.47-2.40 (m, 2H), 2.28 (d, J=7.3 Hz, 3H), 2.17-2.12 (m, 1H), 2.08-2.02 (m, 1H), 2.00 (s, 3H), 1.98 (s, 3H), 1.86 (s, 2H), 1.64-1.48 (m, 2H), 1.33 (d, J=6.9 Hz, 6H), 0.82-0.74 (m, 3H). [M+H]⁺=986.4.

### Example 318: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-3-methyl-2-oxo-2,3-dihydro-1H-benzo[d]imidazol-1-yl)piperidine-2,6-dione

### Step 1: 7-bromo-1-methyl-1,3-dihydro-2H-benzo[d]imidazol-2-one

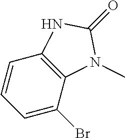

To a solution of 6-bromo-N1-methylbenzene-1,2-diamine (4 g, 19.9 mmol) in CH₃CN (50 mL) was added CDI (6.4 g, 39.8 mmol). The resulting solution was stirred for 6 h at 90° C. under nitrogen atmosphere.

The solid was collected by filtration. This was resulted in 7-bromo-1-methyl-1,3-dihydro-2H-benzo[d]imidazol-2-one (4.1 g, 90.7%). [M+H]⁺=227.0.

### Step 2: 3-(4-bromo-3-methyl-2-oxo-2,3-dihydro-1H-benzo[d]imidazol-1-yl)-1-(4-methoxybenzyl)piperidine-2,6-dione

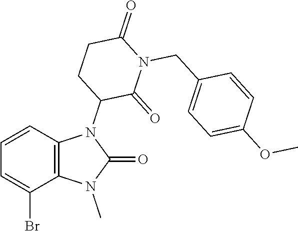 [SMILES: COc1ccc(CN2C(=O)CCC(n3c(=O)n(C)c4c(Br)cccc43)C2=O)cc1]

To a solution of 7-bromo-1-methyl-1,3-dihydro-2H-benzo[d]imidazol-2-one (600 mg, 2.6 mmol) in THF (10 mL) was added t-BuOK (1M in THF, 3.2 mL, 3.1 mmol) dropwise in 10 min at 0° C., the reaction solution was stirred for 30 min at this temperature, then to this was added 1-(4-methoxybenzyl)-2,6-dioxopiperidin-3-yl trifluoromethanesulfonate (1.1 g, 2.9 mmol) in THF (5 mL) dropwise in 10 min. The resulting solution was stirred for 2 h at 0-10 degrees C. The reaction was quenched by the addition of sat.aq. NH4Cl solution, extracted with EtOAc (10 mL×3), combined the organic layer, and washed with brine, dried over anhydrous Na2SO₄, after filtration, the filtrate was concentrated under reduced pressure. The residue was purified by a silica gel column, eluted with PE/EtOAc to afford product (910 mg, 75.2%). [M+H]⁺=458.1.

### Step 3: 3-(4-bromo-3-methyl-2-oxo-2,3-dihydro-1H-benzo[d]imidazol-1-yl)piperidine-2,6-dione

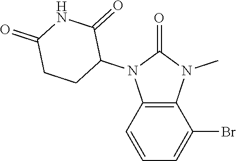 [SMILES: Cn1c(=O)n(C2CCC(=O)NC2=O)c2cccc(Br)c21]

3-(4-bromo-3-methyl-2-oxo-2,3-dihydro-1H-benzo[d]imidazol-1-yl)-1-(4-methoxybenzyl)piperidine-2,6-dione (800 mg, 1.75 mmol) was dissolved in MeSO2H/toluene(2 mL/6 mL). The resulting mixture was stirred for 3 h at 100° C. Solvent was removed and the residue was poured into ice/water. The solid was collected by filtration. 3-(4-bromo-3-methyl-2-oxo-2,3-dihydro-1H-benzo[d]imidazol-1-yl)piperidine-2,6-dione was obtained (510 mg, 86.4%). [M+H]⁺=338.1.

### Step 4: (E)-3-(4-(2-ethoxyvinyl)-3-methyl-2-oxo-2,3-dihydro-1H-benzo[d]imidazol-1-yl)piperidine-2,6-dione

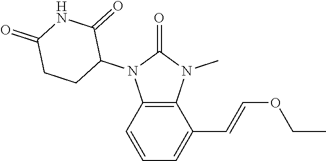 [SMILES: CCO/C=C/c1cccc2c1n(C)c(=O)n2C1CCC(=O)NC1=O]

To a stirred solution of 3-(4-bromo-3-methyl-2-oxo-2,3-dihydro-1H-benzo[d]imidazol-1-yl)piperidine-2,6-dione (250 mg, 0.74 mmol) and (E)-2-(2-ethoxyvinyl)-4,4,5,5-tetramethyl-1,3,2-dioxaborolane (176 mg, 0.89 mmol) in DMF/H₂O (8 mL/2 mL) were added Pd(dtbpf)Cl₂ (48 mg, 0.074 mmol) and CsF (225 mg, 1.48 mmol). The resulting mixture was stirred for 2 h at 80° C. under nitrogen atmosphere. The reaction solution was diluted with water, extracted with EtOAc (10 mL×3). The organic layer was washed with water and brine, dried over anhydrous Na₂SO₄ which was evaporated to dryness. The residue was purified by a silica gel column, eluted with PE/EtOAc=1:1 to afford the product. (180 mg, 73.8%). m/z[M+H]⁺=330.2.

### Step 5: 2-(1-(2,6-dioxopiperidin-3-yl)-3-methyl-2-oxo-2,3-dihydro-1H-benzo[d]imidazol-4-yl)acetaldehyde

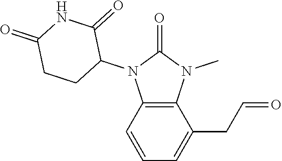 [SMILES: Cn1c(=O)n(C2CCC(=O)NC2=O)c2cccc(CC=O)c21]

(E)-3-(4-(2-ethoxyvinyl)-3-methyl-2-oxo-2,3-dihydro-1H-benzo[d]imidazol-1-yl)piperidine-2,6-dione(180 mg, 0.55 mmol) was dissolved in HCOOH(2 mL). The resulting solution was stirred for 2 h at room temperature. The reaction solution was evaporated to dryness to afford product (125 mg, 75.3%) which was used directly in the next step. m/z[M+H]⁺=302.1.

### Step 6: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-3-methyl-2-oxo-2,3-dihydro-1H-benzo[d]imidazol-1-yl)piperidine-2,6-dione

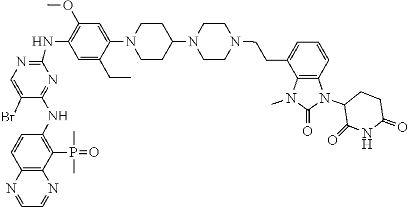 [SMILES: CCc1cc(Nc2ncc(Br)c(Nc3ccc4nccnc4c3P(C)(C)=O)n2)c(OC)cc1N1CCC(N2CCN(CCc3cccc4c3n(C)c(=O)n4C3CCC(=O)NC3=O)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (50 mg, 0.056 mmol), 2-(1-(2,6-dioxopiperidin-3-yl)-3-methyl-2-oxo-2,3-dihydro-1H-benzo[d]imidazol-4-yl)acetaldehyde (17 mg, 0.070 mmol) and NaBH(OAc)₃ (24 mg, 0.11 mmol) in DCE (3 mL) was stirred in a round bottom flask at room temperature for 12 hours. The reaction was diluted with DCM, washed with brine (2×5 mL), dried over Na₂SO₄ and concentrated under vacuum to afford the crude product, which was purified with pre-HPLC (0.1% FA in water: acetonitrile=90:1˜50:50 gradient elution) to give the title product (12 mg, 28%). ¹H NMR (500 MHz, DMSO-d₆) δ 12.58 (s, 1H), 11.03 (s, 1H), 8.78 (d, J=23.1 Hz, 3H), 8.20 (s, 2H), 7.85 (s, 1H), 7.32 (s, 1H), 6.68-7.07 (m, 4H), 5.32 (s, 1H), 3.70 (s, 4H), 3.52 (s, 5H), 2.78-3.11 (m, 8H), 2.60 (d, J=47.5 Hz, 8H), 2.25 (s, 2H), 1.76-2.09 (m, 10H), 1.53 (s, 2H), 0.86 (s, 3H). [M+H]⁺=979.4.

### Example 336: 3-(4-(2-(4-(1-(4-((5-bromo-4-((5-(dimethylphosphoryl)-3-fluoroquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: N-(3-fluoroquinolin-6-yl)-1,1-diphenylmethanimine

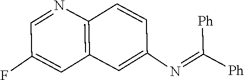 [SMILES: Fc1cnc2ccc(N=C(c3ccccc3)c3ccccc3)cc2c1]

The titled compound (3.8 g, 43%) was synthesized in a manner similar to that in Example 434 step 1 from 6-bromo-3-fluoroquinoline. [M+H]⁺=327.1.

### Step 2: 3-fluoroquinolin-6-amine

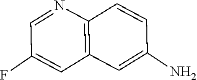 [SMILES: Nc1ccc2ncc(F)cc2c1]

The titled compound (1.8 g, 63%) was synthesized in a manner similar to that in Example 434 step 2 from N-(3-fluoroquinolin-6-yl)-1,1-diphenylmethanimine. [M+H]⁺=163.2.

### Step 3: 3-fluoro-5-iodoquinolin-6-amine

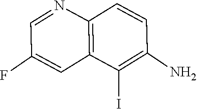 [SMILES: Nc1ccc2ncc(F)cc2c1I]

The titled compound (710 mg, 56%) was synthesized in a manner similar to that in Example 434 step 3 from 3-fluoroquinolin-6-amine. [M+H]⁺=289.3.

### Step 4: (6-amino-3-fluoroquinolin-5-yl)dimethylphosphine oxide

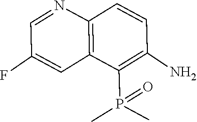 [SMILES: CP(C)(=O)c1c(N)ccc2ncc(F)cc12]

The titled compound (310 mg, 45%) was synthesized in a manner similar to that in Example 434 step 4 from 3-fluoro-5-iodoquinolin-6-amine. [M+H]1=239.1.

### Step 5: (6-((5-bromo-2-chloropyrimidin-4-yl)amino)-3-fluoroquinolin-5-yl)dimethylphosphine oxide

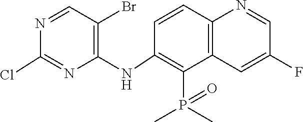 [SMILES: CP(C)(=O)c1c(Nc2nc(Cl)ncc2Br)ccc2ncc(F)cc12]

The titled compound (210 mg, 56%) was synthesized in a manner similar to that in Example 434 step 5 from (6-amino-3-fluoroquinolin-5-yl)dimethylphosphine oxide and 5-bromo-2,4-dichloropyrimidine. [M+H]⁺=429.2.

### Step 6: (6-((5-bromo-2-((5-ethyl-2-methoxy-4-(4-(piperazin-1-yl)piperidin-1-yl)phenyl)amino)pyrimidin-4-yl)amino)-3-fluoroquinolin-5-yl)dimethylphosphine oxide

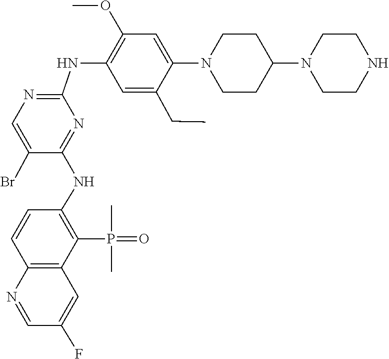 [SMILES: CCc1cc(Nc2ncc(Br)c(Nc3ccc4ncc(F)cc4c3P(C)(C)=O)n2)c(OC)cc1N1CCC(N2CCNCC2)CC1]

The titled compound (130 mg, 45%) was synthesized in a manner similar to that in Example 434 step 6 from (6-((5-bromo-2-chloropyrimidin-4-yl)amino)-3-fluoroquinolin-5-yl)dimethylphosphine oxide and tert-butyl 4-(1-(4-amino-2-ethyl-5-methoxyphenyl)piperidin-4-yl)piperazine-1-carboxylate. [M+H]⁺=711.3.

### Step 7: 3-(4-(2-(4-(1-(4-((5-bromo-4-((5-(dimethylphosphoryl)-3-fluoroquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

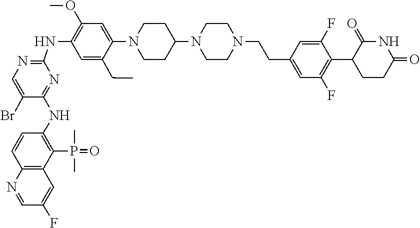 [SMILES: CCc1cc(Nc2ncc(Br)c(Nc3ccc4ncc(F)cc4c3P(C)(C)=O)n2)c(OC)cc1N1CCC(N2CCN(CCc3cc(F)c(C4CCC(=O)NC4=O)c(F)c3)CC2)CC1]

The titled compound (11 mg, 34%) was prepared in a manner similar to that in Example 204 Step 6 from 2-(4-(2,6-dioxopiperidin-3-yl)-3,5-difluorophenyl)acetaldehyde and (6-((5-bromo-2-((5-ethyl-2-methoxy-4-(4-(piperazin-1-yl)piperidin-1-yl)phenyl)amino)pyrimidin-4-yl)amino)-3-fluoroquinolin-5-yl)dimethylphosphine oxide. ¹H NMR (500 MHz, DMSO) δ 11.54 (s, 1H), 10.95 (s, 1H), 8.93 (s, 1H), 8.64 (s, 1H), 8.28 (s, 1H), 8.24 (s, 1H), 8.02 (d, J=9.2 Hz, 1H), 7.96 (s, 1H), 7.36 (s, 1H), 7.03 (d, J=10.0 Hz, 2H), 6.72 (s, 1H), 4.20 (dd, J=12.5, 5.1 Hz, 1H), 3.78 (d, J=20.1 Hz, 3H), 2.91 (d, J=10.6 Hz, 2H), 2.86-2.80 (m, 1H), 2.76 (dd, J=14.9, 7.3 Hz, 3H), 2.63 (t, J=11.0 Hz, 3H), 2.53 (d, J=7.5 Hz, 5H), 2.36 (s, 3H), 2.27 (d, J=11.2 Hz, 1H), 2.20 (s, 2H), 2.16-2.07 (m, 1H), 1.98 (d, J=13.4 Hz, 8H), 1.82 (d, J=10.7 Hz, 2H), 1.52 (dd, J=20.5, 11.4 Hz, 2H), 0.70 (s, 3H). [M+H]⁺=962.3.

### Example 329: 3-(4-(2-(4-(1-(4-((5-bromo-4-((5-(dimethylphosphoryl)-2-(hydroxymethyl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: 6-((5-bromo-2-((4-(4-(4-(4-(2,6-dioxopiperidin-3-yl)-3,5-difluorophenethyl)piperazin-1-yl)piperidin-1-yl)-5-ethyl-2-methoxyphenyl)amino)pyrimidin-4-yl)amino)-5-(dimethylphosphoryl)quinoline-2-carbaldehyde

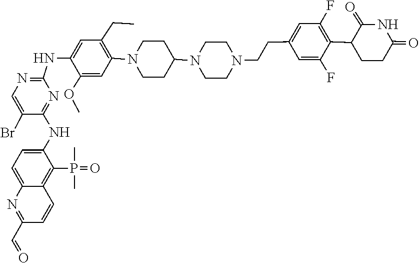 [SMILES: CCc1cc(Nc2ncc(Br)c(Nc3ccc4nc(C=O)ccc4c3P(C)(C)=O)n2)c(OC)cc1N1CCC(N2CCN(CCc3cc(F)c(C4CCC(=O)NC4=O)c(F)c3)CC2)CC1]

To a solution of 3-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione(30 mg, 0.313 mmol) and SeO₂(5.22 mg, 0.047 mmol) in dioxane(2 mL), the mixture was stirred at 100° C. for 2 h. And then water (30 mL) was poured into the mixture. The reaction mixture was filtered and extracted with DCM (2×20 mL). The combined organic phase was washed with brine (2×20 mL), dried over Na₂SO₄, filtered, and concentrated in vacuum. The residue was purified by Prep-TLC (10% MeOH in DCM) to afford product (15 mg, 49.2%). [M+H]⁺=971.3.

### Step 2: 3-(4-(2-(4-(1-(4-((5-bromo-4-((5-(dimethylphosphoryl)-2-(hydroxymethyl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

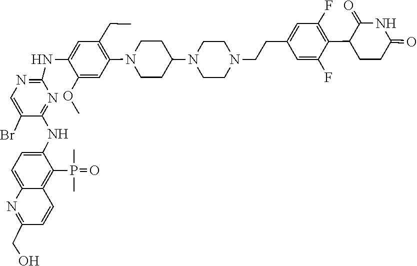 [SMILES: CCc1cc(Nc2ncc(Br)c(Nc3ccc4nc(CO)ccc4c3P(C)(C)=O)n2)c(OC)cc1N1CCC(N2CCN(CCc3cc(F)c(C4CCC(=O)NC4=O)c(F)c3)CC2)CC1]

A mixture of 6-((5-bromo-2-((4-(4-(4-(4-(2,6-dioxopiperidin-3-yl)-3,5-difluorophenethyl)piperazin-1-yl)piperidin-1-yl)-5-ethyl-2-methoxyphenyl)amino)pyrimidin-4-yl)amino)-5-(dimethylphosphoryl)quinoline-2-carbaldehyde (30 mg, 0.313 mmol) and NaOH(25.2 mg, 0.626 mmol) in dioxane(2 mL) was stirred at 100° C. for 2 h. Water (30 mL) was poured into the mixture. The reaction mixture was filtered and extracted with DCM (2×20 mL). The combined organic phase was washed with brine (2×20 mL), dried over Na₂SO₄, filtered, and concentrated in vacuum. The residue was purified by Prep-HPLC with C-18 column chromatography (0.1% FA in water: acetonitrile=90: 10˜ 60: 40 gradient elution) to afford product (3.2 mg, 31.25%). 1H NMR (500 MHz, DMSO) δ 11.82 (s, 1H), 10.88 (s, 1H), 8.58 (d, J=8.9 Hz, 1H), 8.28 (s, 1H), 8.15 (s, 1H), 7.93 (s, 1H), 7.82 (d, J=9.2 Hz, 1H), 7.60 (d, J=8.9 Hz, 1H), 7.31 (s, 1H), 6.96 (d, J=10.0 Hz, 2H), 6.67 (s, 1H), 5.52 (s, 1H), 4.64 (s, 2H), 4.13 (dd, J=12.6, 5.0 Hz, 1H), 3.69 (d, J=5.7 Hz, 4H), 2.87 (d, J=10.9 Hz, 3H), 2.80-2.64 (m, 4H), 2.58 (dd, J=14.7, 7.4 Hz, 3H), 2.54-2.46 (m, 6H), 2.30-2.19 (m, 4H), 2.11-2.00 (m, 2H), 1.93 (t, J=12.6 Hz, 8H), 1.77 (d, J=10.6 Hz, 2H), 1.47 (dd, J=20.2, 11.5 Hz, 2H), 0.71 (s, 3H). [M+H]⁺=974.2.

### Example 409: 3-(5-(3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)azetidin-1-yl)-3,3-dimethyl-2-oxoindolin-1-yl)piperidine-2,6-dione

### Step 1: N-(2,6-bis(benzyloxy)pyridin-3-yl)-1,1-diphenylmethanimine

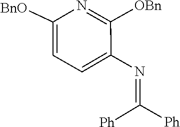 [SMILES: c1ccc(COc2ccc(N=C(c3ccccc3)c3ccccc3)c(OCc3ccccc3)n2)cc1]

To a stirred solution of 2,6-bis(benzyloxy)-3-bromopyridine (30.0 g, 81.0 mmol) and diphenylmethanimine (17.6 g, 97.2 mmol) in dry 1,4-dioxane (400 mL), was added Cs₂CO₃ (52.8 g, 162 mmol) at room temperature. BINAP (5.0 g, 8.1 mmol) and Pd₂(dba)₃ (7.4 g, 8.1 mmol) were added to the mixture at room temperature. The suspension was degassed under vacuum and purged with N₂ three times. Then the mixture was stirred at 100° C. overnight. After cooling to room temperature, the mixture was filtered through a pad of celite, the filtrate was collected and concentrated to afford the residue which was purified by column chromatography (0-5% EA in PE). N-(2,6-bis(benzyloxy)pyridin-3-yl)-1,1-diphenylmethanimine (25.0 g, 65.6%) was obtained. [M+H]⁺=471.2.

### Step 2: 2,6-bis(benzyloxy)pyridin-3-aminium chloride

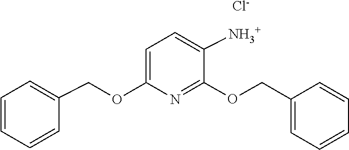 [SMILES: [Cl-] ; [NH3+]c1ccc(OCc2ccccc2)nc1OCc1ccccc1]

To a stirred solution of N-(2,6-bis(benzyloxy)pyridin-3-yl)-1,1-diphenylmethanimine (25.0 g, 53.1 mmol) in THF (200 mL), was added 1N HCl (200 mL) at room temperature. The suspension was stirred at room temperature for 16h, following by rotary evaporation in vacuum to remove the organic solvent. The solid precipitated out during evaporation was collected by filtration, then triturated with PE/EA (200 mL, 10/1) twice, and dried under vacuum to afford product (14.0 g, 76.9%). [M+H]⁺=307.2.

### Step 3: N-(2,6-bis(benzyloxy)pyridin-3-yl)-2-(2,5-dibromophenyl)-2-methylpropanamide

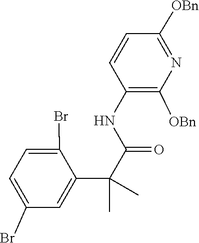 [SMILES: CC(C)(C(=O)Nc1ccc(OCc2ccccc2)nc1OCc1ccccc1)c1cc(Br)ccc1Br]

Into a stirred mixture of 2,6-bis(benzyloxy)pyridin-3-aminium chloride (14.0 g, 40.8 mmol) and 2-(2,5-dibromophenyl)-2-methylpropanoic acid (13.1 g, 40.8 mmol) in dry MeCN (200 mL) under N₂ atmosphere, was added 1-methyl-1H-imidazole (16.8 g, 204 mmol) via a syringe. The mixture was stirred at room temperature for 10 minutes, then a solution of TCFH (13.8 g, 49.1 mmol) in dry MeCN (50 mL) was added into the mixture via a syringe. The mixture was stirred at 50° C. under N₂ atmosphere for 4 hrs. After cooling to room temperature, the reaction was quenched by addition of water (2 mL), then the mixture was concentrated in vacuum by an evaporator to afford the residue which was purified by column chromatography (5% EA in PE) to afford product (15.0 g, 60.2%). [M+H]⁺=611.2.

### Step 4: 1-(2,6-bis(benzyloxy)pyridin-3-yl)-5-bromo-3,3-dimethylindolin-2-one

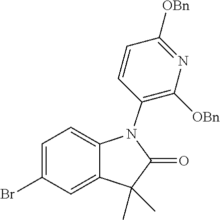 [SMILES: CC1(C)C(=O)N(c2ccc(OCc3ccccc3)nc2OCc2ccccc2)c2ccc(Br)cc21]

To a stirred mixture of N-(2,6-bis(benzyloxy)pyridin-3-yl)-2-(2,5-dibromophenyl)-2-methylpropanamide (10.0 g, 16.4 mmol) and K₂CO₃ (11.3 g, 81.9 mmol) in NMP (150 mL), was added CuCl (1.62 g, 16.4 mmol). The suspension was degassed under vacuum and purged with N₂ three times. Pentane-2,4-dione (3.28 g, 32.8 mmol) was added into the mixture via syringe. The suspension was stirred at 85° C. under N₂ atmosphere for 2 hrs. After cooling to room temperature, the mixture was poured into EA (500 mL), and washed with brine (200 mL). The organic layer was then dried over anhydrous Na₂SO₄ and concentrated in vacuum. The residue was purified by column chromatography (7% EA in PE) to afford product (6.0 g, 69.2%). [M+H]⁺=529.2.

### Step 5: ethyl 1-(1-(2,6-bis(benzyloxy)pyridin-3-yl)-3,3-dimethyl-2-oxoindolin-5-yl)azetidine-3-carboxylate

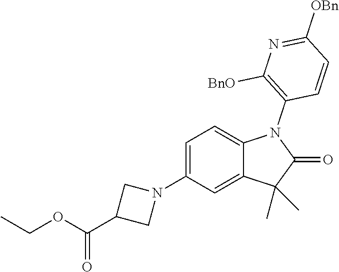 [SMILES: CCOC(=O)C1CN(c2ccc3c(c2)C(C)(C)C(=O)N3c2ccc(OCc3ccccc3)nc2OCc2ccccc2)C1]

A mixture of 1-(2,6-bis(benzyloxy)pyridin-3-yl)-5-bromo-3,3-dimethylindolin-2-one (1.0 g, 1.889 mmol) and ethyl azetidine-3-carboxylate hydrochloride (375.4 mg, 2.267 mmol), Cs₂CO₃ (2.5 g, 7.556 mmol), RuPhos (88.1 mg, 0.189 mmol), Pd₂(dba)₃ (173.0 mg, 0.189 mmol) in dioxane (20 mL) was stirred overnight at 100° C. under nitrogen atmosphere. The resulting mixture was filtered, the filter cake was washed with EtOAc (150 mL). The filtrate was concentrated under reduced pressure. The residue was purified by Prep-TLC (PE/EtOAc 1:1). Ethyl 1-(1-(2,6-bis(benzyloxy)pyridin-3-yl)-3,3-dimethyl-2-oxoindolin-5-yl)azetidine-3-carboxylate (290.0 mg, 26.5%) was obtained. [M+H]⁺=578.3.

### Step 6: 1-(1-(2,6-bis(benzyloxy)pyridin-3-yl)-3,3-dimethyl-2-oxoindolin-5-yl)azetidine-3-carboxylic acid

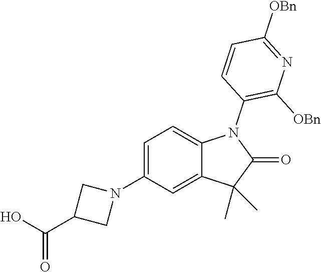 [SMILES: CC1(C)C(=O)N(c2ccc(OCc3ccccc3)nc2OCc2ccccc2)c2ccc(N3CC(C(=O)O)C3)cc21]

A mixture of ethyl 1-(1-(2,6-bis(benzyloxy)pyridin-3-yl)-3,3-dimethyl-2-oxoindolin-5-yl)azetidine-3-carboxylate (290.0 mg, 0.502 mmol) and NaOH (180.7 mg, 4.518 mmol) in MeOH (40 mL), THF (8 mL) and H₂O (8 mL) was stirred overnight at room temperature. The mixture was acidified to pH<7 with 2N HCl (aq.). The resulting mixture was extracted with EtOAc (300 mL). The combined organic layers were washed with brine, dried over anhydrous Na₂SO₄. After filtration, the filtrate was concentrated under reduced pressure. 1-(1-(2,6-bis(benzyloxy)pyridin-3-yl)-3,3-dimethyl-2-oxoindolin-5-yl)azetidine-3-carboxylic acid (270.0 mg, 97.8%) was obtained. [M+H]⁺=550.2.

### Step 7: 1-(1-(2,6-dioxopiperidin-3-yl)-3,3-dimethyl-2-oxoindolin-5-yl)azetidine-3-carboxylic acid

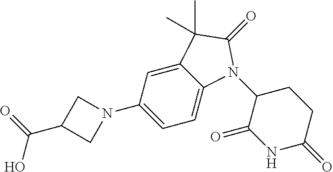 [SMILES: CC1(C)C(=O)N(C2CCC(=O)NC2=O)c2ccc(N3CC(C(=O)O)C3)cc21]

A mixture of 1-(1-(2,6-bis(benzyloxy)pyridin-3-yl)-3,3-dimethyl-2-oxoindolin-5-yl)azetidine-3-carboxylic acid (270.0 mg, 0.491 mmol) and Pd/C (200.0 mg, 1.879 mmol) in EtOH (10 mL) and DCM (2 mL) was stirred overnight at 50° C. under hydrogen atmosphere. The resulting mixture was filtered, the filter cake was washed with EtOH (150 mL). The filtrate was concentrated under reduced pressure. The residue was purified by Prep-TLC (pure EtOAc with 0.5% TFA). 1-[1-(2,6-dioxopiperidin-3-yl)-3,3-dimethyl-2-oxoindol-5-yl]azetidine-3-carboxylic acid (84.4 mg, 46.2%) was obtained. [M+H]⁺=372.2.

### Step 8: 3-(5-(3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)azetidin-1-yl)-3,3-dimethyl-2-oxoindolin-1-yl)piperidine-2,6-dione

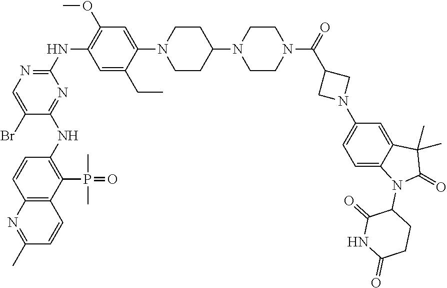 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4ccc5c(c4)C(C)(C)C(=O)N5C4CCC(=O)NC4=O)C3)CC2)CC1]

To a solution of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide(50 mg, 0.0708 mmol), HATU (41 mg, 0.106 mmol) and 1-(1-(2,6-dioxopiperidin-3-yl)-3,3-dimethyl-2-oxoindolin-5-yl)azetidine-3-carboxylic acid (32 mg, 0.085 mmol) in DMF (2 mL) was added DIEA (18.2 mg 0.142 mmol) at 0° C. Then the mixture was stirred at RT for 5 h. Water (10 mL) was poured into the mixture. The reaction mixture was extracted with DCM (2×20 mL). The combined organic phase was washed with brine (2×20 mL), dried over Na₂SO₄, filtered, and concentrated in vacuum. The residue was purified by Prep-HPLC with C-18 column chromatography (0.1% FA in water: acetonitrile=90: 10˜ 60: 40 gradient elution) to afford the product (21 mg, 28.03%). ¹H NMR (500 MHz, DMSO) δ 11.70 (s, 1H), 10.96 (s, 1H), 8.50 (d, J=8.8 Hz, 1H), 8.23 (s, 1H), 8.13 (d, J=11.5 Hz, 1H), 7.91 (s, 1H), 7.80 (d, J=9.2 Hz, 1H), 7.36 (d, J=8.9 Hz, 1H), 6.77-6.66 (m, 2H), 6.53 (d, J=2.2 Hz, 1H), 6.22 (dd, J=8.4, 2.1 Hz, 1H), 5.07 (s, 1H), 3.93 (dd, J=17.5, 10.2 Hz, 2H), 3.82-3.72 (m, 3H), 3.70 (d, J=9.5 Hz, 3H), 3.42 (s, 3H), 2.88 (d, J=10.6 Hz, 2H), 2.83-2.70 (m, 1H), 2.68-2.56 (m, 6H), 2.55-2.47 (m, 5H), 2.31 (dd, J=15.5, 6.5 Hz, 1H), 2.23 (d, J=7.0 Hz, 2H), 1.93 (t, J=12.4 Hz, 6H), 1.85 (d, J=5.0 Hz, 1H), 1.76 (d, J=10.7 Hz, 2H), 1.50 (dd, J=20.3, 11.6 Hz, 2H), 1.20 (d, J=3.3 Hz, 6H), 0.75 (m, 3H).[M+H]⁺=1060.1.

### Example 219: 3-(5-(4-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazine-1-carbonyl)piperidin-1-yl)-3,3-dimethyl-2-oxoindolin-1-yl)piperidine-2,6-dione

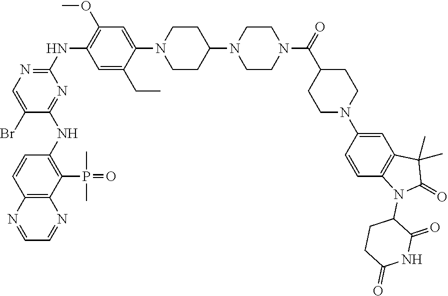 [SMILES: CCc1cc(Nc2ncc(Br)c(Nc3ccc4nccnc4c3P(C)(C)=O)n2)c(OC)cc1N1CCC(N2CCN(C(=O)C3CCN(c4ccc5c(c4)C(C)(C)C(=O)N5C4CCC(=O)NC4=O)CC3)CC2)CC1]

The titled compound was prepared in a manner similar to that in Example 409. 1H NMR (500 MHz, DMSO) δ 12.65 (s, 1H), 11.04 (s, 1H), 8.86 (dd, J=8.9, 1.8 Hz, 2H), 8.82 (s, 1H), 8.27 (s, 2H), 7.90 (d, J=9.1 Hz, 1H), 7.38 (s, 1H), 7.10 (d, J=1.9 Hz, 1H), 6.78 (dd, J=21.0, 10.4 Hz, 3H), 5.16 (s, 1H), 3.77 (s, 3H), 3.60-3.53 (m, 4H), 3.49 (d, J=11.4 Hz, 2H), 3.30 (s, 2H), 3.03 (d, J=9.6 Hz, 2H), 2.85 (d, J=12.8 Hz, 1H), 2.74-2.68 (m, 5H), 2.62-2.57 (m, 3H), 2.48-2.46 (m, 3H), 2.02 (d, J=14.4 Hz, 7H), 1.93-1.90 (m, 1H), 1.85 (s, 2H), 1.71 (s, 4H), 1.64-1.59 (m, 2H), 1.28 (d, J=3.1 Hz, 6H), 0.93 (t, J=6.9 Hz, 3H); [M+H]⁺=1075.7.

### Example 317: 3-(5-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)cyclobutyl)-3,3-dimethyl-2-oxoindolin-1-yl)piperidine-2,6-dione

### Step 1: 5-(3-(benzyloxy)cyclobutyl)-1-(2,6-bis(benzyloxy)pyridin-3-yl)-3 ,3-dimethylindolin-2-one

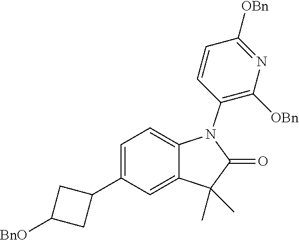 [SMILES: CC1(C)C(=O)N(c2ccc(OCc3ccccc3)nc2OCc2ccccc2)c2ccc(C3CC(OCc4ccccc4)C3)cc21]

A 50 mL three-neck round bottom flask equipped with a magnetic stirrer were charged with 1-(2,6-bis(benzyloxy)pyridin-3-yl)-5-bromo-3,3-dimethylindolin-2-one (1.00 g, 1.89 mmol), ((3-iodocyclobutoxy)methyl)benzene (815 mg, 2.83 mmol), NiI₂ (118 mg, 0.38 mmol), picolinimidamide (46 mg, 0.38 mmol), NaI (142 mg, 0.95 mmol), Mn (325 mesh, 312 mg, 5.67 mmol) and DMA (20 mL). The resulting mixture was degassed under vacuum and purged with N₂ three times. Into this stirred mixture, a solution of TFA (70 μL, 0.95 mmol) in DMA (1 mL) was added dropwise by a syringe. The suspension was stirred at 100° C. for 3 hrs. After cooled to room temperature, the suspension was poured into EA (200 mL), then washed with brine (100 mL), water (3×100 mL) and brine (100 mL) in turn, dried over anhydrous Na₂SO₄, concentrated in vacuum. The residue was purified by prep TLC (PE/EA=1:1) to afford the product (450 mg, 39.0%). [M+H]⁺=611.5.

### Step 2: 3-(5-(3-hydroxycyclobutyl)-3,3-dimethyl-2-oxoindolin-1-yl)piperidine-2,6-dione

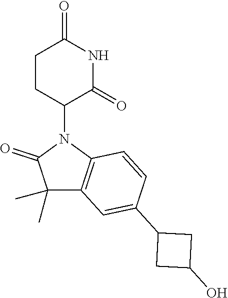 [SMILES: CC1(C)C(=O)N(C2CCC(=O)NC2=O)c2ccc(C3CC(O)C3)cc21]

A 50 mL round bottom flask equipped with a magnetic stirrer were charged with 5-(3-(benzyloxy)cyclobutyl)-1-(2,6-bis(benzyloxy)pyridin-3-yl)-3,3-dimethylindolin-2-one (430 mg, 0.70 mmol), Pd/C (10 wt %, 400 mg), EtOH (20 mL) and AcOH (200 μL). The suspension was degassed under vacuum and purged with H₂ five times by a H₂ balloon. The mixture was stirred at 50° C. for 2 days. After cooling to room temperature, the mixture was poured into a mixture of DCM (30 mL) and EtOH (10 mL), and filtered through a pad of celite. The filtrate was collected and concentrated in vacuum, the residue was dissolved in DCM (20 mL), re-concentrated and dried in vacuum to afford the product (270 mg, 112%), which is used directly for next step without further purification. [M+H]⁺=343.1.

### Step 3: 3-(3,3-dimethyl-2-oxo-5-(3-oxocyclobutyl) indolin-1-yl)piperidine-2,6-dione

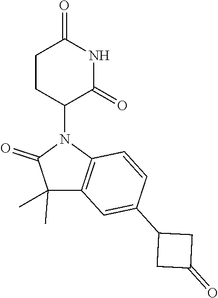 [SMILES: CC1(C)C(=O)N(C2CCC(=O)NC2=O)c2ccc(C3CC(=O)C3)cc21]

A25 mL round bottom flask equipped with a magnetic stirrer was charged with 3-(5-(3-hydroxycyclobutyl)-3,3-dimethyl-2-oxoindolin-1-yl)piperidine-2,6-dione (260 mg, 0.76 mmol), DCM (4.5 mL), dry THF (1.5 mL) and NaHCO₃ (638 mg,). To this suspension, Dess-martin periodinane (419 mg, 099 mmol) was added portionwise. The mixture was stirred at room temperature for 2 hrs and then poured into a mixture of DCM (50 mL) and dry THF (50 mL). The mixture was filtered through a pad of celite, the filtrate was collected and concentrated in vacuum. The residue was purified by prep TLC (pure EA) to give the crude product, which is further purified by trituration with PE. 3-(3,3-dimethyl-2-oxo-5-(3-oxocyclobutyl) indolin-1-yl)piperidine-2,6-dione was obtained (140 mg, 54.1%). [M+H]⁺=341.2.

### Step 4: 3-(5-(3-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)cyclobutyl)-3,3-dimethyl-2-oxoindolin-1-yl)piperidine-2,6-dione

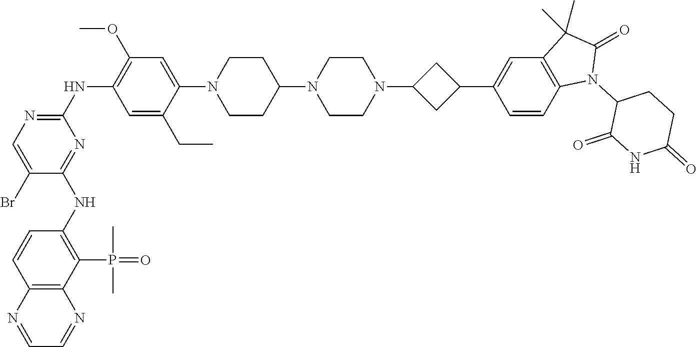 [SMILES: CCc1cc(Nc2ncc(Br)c(Nc3ccc4nccnc4c3P(C)(C)=O)n2)c(OC)cc1N1CCC(N2CCN(C3CC(c4ccc5c(c4)C(C)(C)C(=O)N5C4CCC(=O)NC4=O)C3)CC2)CC1]

To a solution of (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide (40 mg, 0.058 mmol), 3-(3,3-dimethyl-2-oxo-5-(3-oxocyclobutyl) indolin-1-yl)piperidine-2,6-dione(17 mg, 0.070 mmol) and Ti(iPrO)₄ (2 drops) in DCE(3 mL) was added NaBH(OAc)₃ (11 mg, 0.17 mmol). The resulting solution was stirred at room temperature for 12 hours. The reaction was diluted with DCM, washed with brine (2×5 mL), dried over Na2SO₄ and concentrated under vacuum to afford the crude product, which was purified with pre-HPLC (0.1% FA in water: acetonitrile=90:1˜50:50 gradient elution) to give the title product (12 mg, 28%). ¹H NMR (500 MHz, DMSO-d₆) δ 12.58 (s, 1H), 10.99 (s, 1H), 8.77 (d, J=22.4 Hz, 3H), 8.27 (d, J=69.5 Hz, 3H), 7.83 (s, 1H), 7.25 (d, J=56.7 Hz, 2H), 7.04 (d, J=43.6 Hz, 1H), 6.75 (t, J=32.0 Hz, 2H), 5.13 (s, 1H), 3.65 (s, 3H), 2.90-3.06 (m, 7H), 2.48-2.64 (m, 7H), 2.18-2.35 (m, 6H), 1.69-1.97 (m, 12H), 1.46-1.57 (m, 2H), 1.23 (s, 6H), 0.86 (s, 3H). [M+H]⁺=1018.4.

### Example 413: 3-(6-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-3,3-dimethyl-2-oxoindolin-1-yl)piperidine-2,6-dione

### Step 1: 1-(tert-butyl) 3-methyl 2-(4-bromo-2-nitrophenyl)malonate

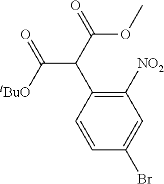 [SMILES: COC(=O)C(C(=O)OC(C)(C)C)c1ccc(Br)cc1[N+](=O)[O-]]

To a stirred solution of 1-tert-butyl 3-methyl propanedioate (30.88 g, 177.275 mmol) and 4-bromo-1-fluoro-2-nitrobenzene (32.50 g, 147.729 mmol) in DMF (200.00 mL) was added Cs₂CO₃ (96.27 g, 295.459 mmol) dropwise at 0° C. under nitrogen atmosphere. The resulting mixture was stirred for 16 h at 70° C. The resulting mixture was filtered, and the filter cake was washed with EtOAc (3×500 mL). The solution was acidified to pH 7-8 with HCl (aq., 1 M). The resulting mixture was diluted with water (1.5 L). The resulting solution was extracted with EA (3×1000 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (40:1) to afford product (47.1 g, 85.21%).[M+H]⁺=373.9

### Step 2: methyl 2-(4-bromo-2-nitrophenyl)acetate

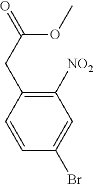

To a stirred solution of 1-(tert-butyl) 3-methyl 2-(4-bromo-2-nitrophenyl)malonate (46.10 g, 123.200 mmol) in toluene (200.00 mL) was added TsOH·H₂O (11.72 g, 61.600 mmol) at 110° C. under nitrogen atmosphere for 16 h. The resulting mixture was concentrated under vacuum. The resulting mixture was extracted with EtOAc (3×200 mL). The combined organic layers were washed with aqueous NaHCO₃ (3×100 mL), dried over anhydrous Na₂SO₄. After filtration, the filtrate was concentrated under reduced pressure. The residue was purified by silica gel column chromatography, eluted with 1˜ 30% EtOAc in PE to afford product (26.5 g, 78.48%). [M+H]⁺=273.9

### Step 3: methyl 2-(4-bromo-2-nitrophenyl)-2-methylpropanoate

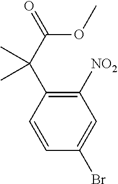 [SMILES: COC(=O)C(C)(C)c1ccc(Br)cc1[N+](=O)[O-]]

To a stirred solution of methyl 2-(4-bromo-2-nitrophenyl)acetate (12.00 g, 43.784 mmol) in ACN (240.00 mL) was added Cs₂CO₃ (28.53 g, 87.569 mmol) at 0° C. under nitrogen atmosphere at ambient temperature. To the mixture was added CH₃I (31.07 g, 218.922 mmol) dropwise over 20 min. The resulting mixture was stirred for 16 h at 80° C. After cooling down to ambient temperature, the resulting mixture was filtered, and the filtrate was washed with EtOAc (3×200 mL). The combined organic layers were concentrated under reduced pressure. The residue was purified by silica gel column chromatography, eluted with 1˜ 40% EtOAc in PE to afford product (5.6 g, 42.33%). [M+H]⁺=301.9.

### Step 4: 6-bromo-3,3-dimethylindolin-2-one

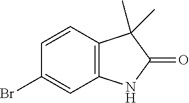

To a stirred solution of methyl 2-(4-bromo-2-nitrophenyl)-2-methylpropanoate (5.50 g, 10.923 mmol) in AcOH (50.00 mL) was added Fe (2.44 g, 43.692 mmol) at room temperature. The resulting mixture was stirred for 2 h at ambient temperature. The resulting mixture was concentrated under reduced pressure. The residue was purified by silica gel column chromatography, eluted with 10˜ 50% EtOAc in PE to afford product (1.95 g, 74.36%). [M+H]⁺=239.9.

### Step 5: 3-(6-bromo-3,3-dimethyl-2-oxoindolin-1-yl)-1-(4-methoxybenzyl)piperidine-2,6-dione

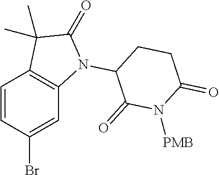

To a stirred mixture of 6-bromo-3,3-dimethylindolin-2-one (1.80 g, 7.497 mmol) in THF (160.00 mL) was added t-BuOK (0.93 g, 8.247 mmol) in portions at 0° C. under nitrogen atmosphere. The resulting mixture was stirred for 1 h at room temperature under nitrogen atmosphere. To the mixture was added 1-(4-methoxybenzyl)-2,6-dioxopiperidin-3-yl trifluoromethanesulfonate (This intermediate was prepared according the same manner described in WO 2020113233A1) (3.14 g, 8.247 mmol) in THF (20 mL) at 0° C. The resulting mixture was stirred for additional 2 h at room temperature. The reaction was quenched with sat. aq. NH₄Cl (30 mL) at 0° C. and diluted with water (1 L). The resulting mixture was extracted with EtOAc (3×500 mL). The combined organic layers were washed with brine (2×200 mL), dried over anhydrous Na₂SO₄. After filtration, the filtrate was concentrated under reduced pressure. The residue was purified by silica gel column chromatography, eluted with PE/EtOAc (1:1) to afford product (2.6 g, 73.58%). [M+H]⁺=471.1.

### Step 6: 3-(6-bromo-3,3-dimethyl-2-oxoindolin-1-yl)piperidine-2,6-dione

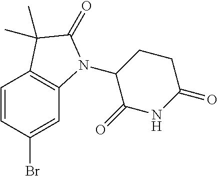 [SMILES: CC1(C)C(=O)N(C2CCC(=O)NC2=O)c2cc(Br)ccc21]

A solution of 3-(6-bromo-3,3-dimethyl-2-oxoindolin-1-yl)-1-(4-methoxybenzyl)piperidine-2,6-dione (2.60 g, 5.516 mmol) and CH₃SO₃H (10.60 g, 110.321 mmol) in Toluene (260.00 mL) was stirred for 2 h at 110° C. under nitrogen atmosphere. The resulting mixture was concentrated under reduced pressure. The resulting mixture was diluted with EtOAc (400 mL). The combined organic layers were washed with brine (3×200 mL), dried over anhydrous Na₂SO₄. After filtration, the filtrate was concentrated under reduced pressure. The residue was purified by silica gel column chromatography, eluted with 1˜ 50% EtOAc in PE to afford 3-(6-bromo-3,3-dimethyl-2-oxoindolin-1-yl)piperidine-2,6-dione (1.4 g, 72.27%). [M+H]⁺=351.05.

### Step 7: 3-(6-allyl-3,3-dimethyl-2-oxoindolin-1-yl)piperidine-2,6-dione

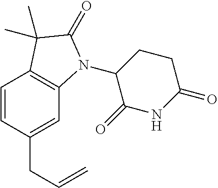 [SMILES: C=CCc1ccc2c(c1)N(C1CCC(=O)NC1=O)C(=O)C2(C)C]

To a stirred solution of 3-(6-bromo-3,3-dimethyl-2-oxoindolin-1-yl)piperidine-2,6-dione (800.00 mg, 2.278 mmol) and tributyl(prop-2-en-1-yl)stannane (1131.39 mg, 3.417 mmol) in DMF (50.00 mL) was added Pd(PPh₃)₂Cl₂ (239.83 mg, 0.342 mmol) at ambient temperature under nitrogen atmosphere. The resulting mixture was stirred for 2h at 105° C. under nitrogen atmosphere. The resulting mixture was diluted with water (200 mL). The resulting mixture was extracted with EtOAc (3×100 mL). The combined organic layers were washed with brine (3×100 mL), dried over anhydrous Na₂SO₄. After filtration, the filtrate was concentrated under reduced pressure. The residue was purified by silica gel column chromatography, eluted with 1˜ 40% EtOAc in petroleum ether to afford product (500 mg, 70.27%). [M+H]⁺=313.20

### Step 8: 2-(1-(2,6-dioxopiperidin-3-yl)-3,3-dimethyl-2-oxoindolin-6-yl)acetaldehyde

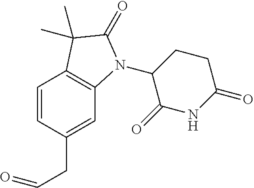 [SMILES: CC1(C)C(=O)N(C2CCC(=O)NC2=O)c2cc(CC=O)ccc21]

To a stirred solution of 3-(6-allyl-3,3-dimethyl-2-oxoindolin-1-yl)piperidine-2,6-dione (500.00 mg, 1.601 mmol) in dioxane (60.00 mL) and water (12.00 mL) were added K₂OsO₄·2H₂O (88.47 mg, 0.240 mmol) and NaIO₄ (1369.48 mg, 6.403 mmol) at 0° C. under nitrogen atmosphere. The resulting mixture was stirred for 12 h at room temperature under nitrogen atmosphere. The resulting mixture was diluted with water (20 mL). The resulting mixture was extracted with EtOAc (3×200 mL). The resulting mixture was concentrated under reduced pressure. The residue was purified by reverse flash chromatography with the following conditions: column, C18; mobile phase, ACN in water (0.1% FA), 10% to 50% gradient in 10 min; Detector, UV 220 nm; to afford product (300.1 mg, 59.64%). [M+H]⁺=315.20.

### Step 9: 3-(6-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-3,3-dimethyl-2-oxoindolin-1-yl)piperidine-2,6-dione

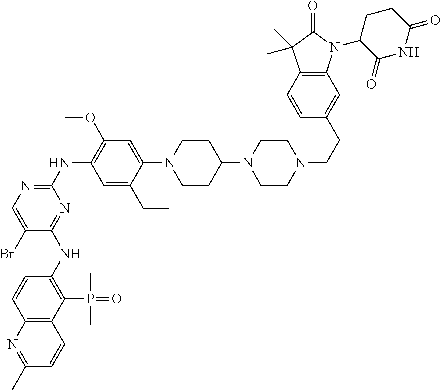 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)N(C3CCC(=O)NC3=O)C(=O)C4(C)C)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide(50.0 mg, 0.071 mmol), 2-(1-(2,6-dioxopiperidin-3-yl)-3,3-dimethyl-2-oxoindolin-6-yl)acetaldehyde (22.2 mg, 0.071 mmol) and AcOH (0.1 mL) in a mixture of DCM (4 mL) and MeOH (4 mL) was stirred at rt for 2 h. Then NaBH(OAc)₃ (111 mg, 0.425 mmol) was added to the mixture. The resulting mixture was stirred at rt for 2 h. The mixture was diluted with water (50 mL), extracted with DCM (3×50 mL). The combined organic layers were dried over Na2SO₄, filtered and concentrated under vacuum. The residue was purified by Prep-HPLC with the following conditions: Anal Column: SunFire PrepC18 OBD, 5 μm, 19×150 mm, Column Temp: Room temperature, Mobile Phase A: H₂O (0.1% FA), Mobile Phase B: ACN, Gradient Table: Mobile Phase B (15-35%), Time (min):0-17 min to afford the product (10.8 mg, 15.2%). ¹H NMR (500 MHz, DMSO) δ 11.77 (s, 1H), 11.06 (s, 1H), 8.56 (d, J=8.9 Hz, 1H), 8.30 (m, 1H), 8.20 (d, J=13.1 Hz, 3H), 7.98 (s, 1H), 7.87 (d, J=9.2 Hz, 1H), 7.42 (d, J=8.9 Hz, 1H), 7.37 (s, 1H), 7.18 (t, J=7.9 Hz, 1H), 6.91 (d, J=7.7 Hz, 1H), 6.80-6.87 (m, 1H), 6.74 (s, 1H), 5.22 (s, 1H), 3.77 (d, J=12.0 Hz, 3H), 3.00-2.75 (m, 6H), 2.72-2.52 (m, 15H), 2.30-2.60 (m, 3H), 1.98 (d, J=13.3 Hz, 8H), 1.84 (d, J=11.2 Hz, 2H), 1.45-1.59 (m, 2H), 1.38 (d, J=4.2 Hz, 7H), 0.78 (s, 3H). [M+H]⁺=1004.4.

### Example 426: 3-(5-(3-((4-(1-(4-((5-bromo-4-((2-cyclopropyl-5-(dimethylphosphoryl)quinolin-6-yl)amino)pyrimidin-2-yl)amino)-2-ethyl-5-methoxyphenyl)piperidin-4-yl)piperazin-1-yl)methyl)azetidin-1-yl)-1-oxoisoindolin-2-yl)piperidine-2,6-dione

### Step 1: 2-chloroquinolin-6-amine

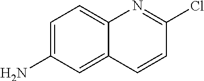 [SMILES: Nc1ccc2nc(Cl)ccc2c1]

To a solution of 2-chloro-6-nitroquinoline (5 g, 24.03 mmol) in EtOH (60 mL) and water (25 mL) was added iron powder (6.73 g, 120 mmol) and NH4Cl (6.36 g, 120 mmol). The mixture was stirred at 70° C. for 3 hrs. After cooling to r.t, the solid was filtered off, the filtrate was extracted with DCM (70 mL×3). The combined organic phase was washed with brine (60 mL), dried over Na₂SO₄, filtered and concentrated in vacuum. 2-chloroquinolin-6-amine (4 g, 93%) was obtained. [M+H]⁺=179.1.

### Step 2: 2-chloro-5-iodoquinolin-6-amine

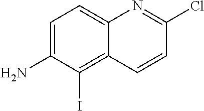 [SMILES: Nc1ccc2nc(Cl)ccc2c1I]

To a solution of 2-chloroquinolin-6-amine (4 g, 22.3 mmol) in HOAc (20 mL) was added ICl (4.24 g, 33.45 mmol). The mixture was stirred at 20° C. for 3 hrs and then sat. aq. Na2CO₃ solution was added to PH=8-9, the resulting mixture was extracted with EtOAc (70 mL×3). The combined organic phase was washed with brine (60 mL), dried over Na₂SO₄, filtered and concentrated in vacuum. 5-iodo-2-(trifluoromethyl)quinolin-6-amine (6.7 g, 88.6%) was obtained. [M+H]⁺=304.9.

### Step 3: (6-amino-2-chloroquinolin-5-yl)dimethylphosphine oxide

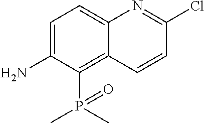 [SMILES: CP(C)(=O)c1c(N)ccc2nc(Cl)ccc12]

To a mixture of 2-chloro-5-iodoquinolin-6-amine (6.5 g, 21.3 mmol), dimethylphosphine oxide (2.43 g, 32 mmol), Pd(OAc)2 (477 mg, 2.13 mmol), Xantphos (2.46 g, 4.26 mmol), K3PO4 (9.03 g, 42.6 mmol) in dioxane (110 mL) was stirred at 100° C. under N2 atmosphere for 18 hrs. After cooling to r.t, the reaction mixture was filtered, the filtrate was concentrated in vacuo. The residue was purified by silica gel column (DCM:CH3OH=15:1). (6-amino-2-chloroquinolin-5-yl)dimethylphosphine oxide (5.1 g, 94%) was obtained. [M+H]⁺=255.

### Step 4: (6-amino-2-cyclopropylquinolin-5-yl)dimethylphosphine oxide

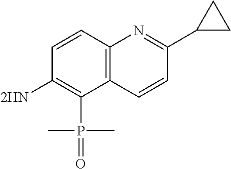

To a solution of (6-amino-2-chloroquinolin-5-yl)dimethylphosphine oxide (2.0 g, 7.9 mmol), cyclopropylboronic acid (2.04 g, 23.7 mmol), Pd(dppf)Cl₂ (0.58 g, 0.79 mmol) and Na2CO₃ (1.7 g, 15.8 mmol) in a mixture of dioxane (20 mL) and H₂O (4 mL) was stirred at 100° C. for overnight. The mixture was concentrated in vacuum. The residue was purified by column chromatography to afford product (380 mg, 19.0%). [M+H]⁺=261.1.

### Step 5: (6-((5-bromo-2-chloropyrimidin-4-yl)amino)-2-cyclopropylquinolin-5-yl)dimethylphosphine oxide

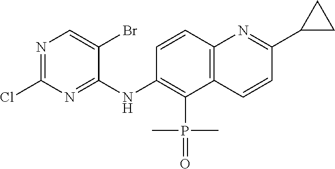 [SMILES: CP(C)(=O)c1c(Nc2nc(Cl)ncc2Br)ccc2nc(C3CC3)ccc12]

The titled compound (550 mg, 86%) was prepared in a manner similar to that in Example 200 step 13 from (6-amino-2-cyclopropylquinolin-5-yl)dimethylphosphine oxid and 5-bromo-2,4-dichloropyrimidine. [M+H]⁺=451.0.

### Step 6: (6-((5-bromo-2-((5-ethyl-2-methoxy-4-(4-(piperazin-1-yl)piperidin-1-yl)phenyl)amino)pyrimidin-4-yl)amino)-2-cyclopropylquinolin-5-yl)dimethylphosphine oxide

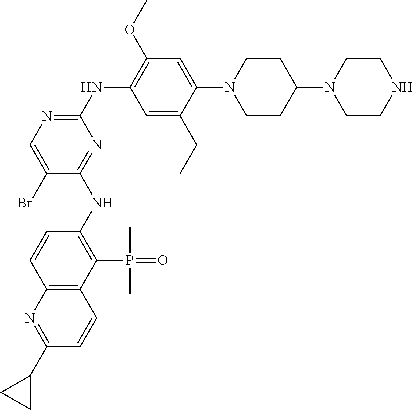 [SMILES: CCc1cc(Nc2ncc(Br)c(Nc3ccc4nc(C5CC5)ccc4c3P(C)(C)=O)n2)c(OC)cc1N1CCC(N2CCNCC2)CC1]

The titled compound (550 mg, 86%) was prepared in a manner similar to that in Example 200 step 14 from (6-((5-bromo-2-chloropyrimidin-4-yl)amino)-2-cyclopropylquinolin-5-yl)dimethylphosphine oxide and tert-butyl 4-(1-(4-amino-2-ethyl-5-methoxyphenyl)piperidin-4-yl)piperazine-1-carboxylate. [M+H]⁺=733.3.

### Step 7: 3-(5-(3-((4-(1-(4-((5-bromo-4-((2-cyclopropyl-5-(dimethylphosphoryl)quinolin-6-yl)amino)pyrimidin-2-yl)amino)-2-ethyl-5-methoxyphenyl)piperidin-4-yl)piperazin-1-yl)methyl)azetidin-1-yl)-1-oxoisoindolin-2-yl)piperidine-2,6-dione

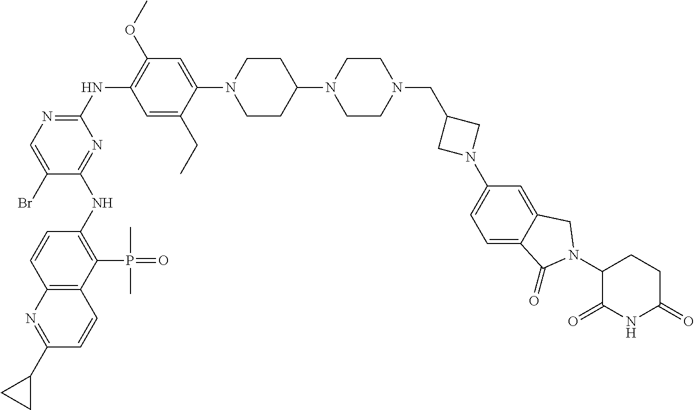 [SMILES: CCc1cc(Nc2ncc(Br)c(Nc3ccc4nc(C5CC5)ccc4c3P(C)(C)=O)n2)c(OC)cc1N1CCC(N2CCN(CC3CN(c4ccc5c(c4)CN(C4CCC(=O)NC4=O)C5=O)C3)CC2)CC1]

The titled compound was prepared in a manner similar to that in Example 207 step 5 from (6-((5-bromo-2-((5-ethyl-2-methoxy-4-(4-(piperazin-1-yl)piperidin-1-yl)phenyl)amino)pyrimidin-4-yl)amino)-2-cyclopropylquinolin-5-yl)dimethylphosphine oxide and (1-(2-(2,6-dioxopiperidin-3-yl)-1-oxoisoindolin-5-yl)azetidin-3-yl)methyl methanesulfonate (this material was obtained through the similar method of example 237). ¹H NMR (500 MHz, DMSO) δ 11.75 (s, 1H), 10.93 (s, 1H), 8.49 (d, J=8.8 Hz, 1H), 8.19 (s, 2H), 8.03 (s, 1H), 7.74 (d, J=9.2 Hz, 1H), 7.48 (d, J=8.3 Hz, 1H), 7.44 (d, J=8.9 Hz, 1H), 7.28 (s, 1H), 6.76 (s, 1H), 6.51 (s, 1H), 6.47 (d, J=8.2 Hz, 1H), 5.03 (dd, J=13.2, 5.1 Hz, 1H), 4.30 (d, J=16.9 Hz, 1H), 4.17 (d, J=16.9 Hz, 1H), 4.02 (t, J=7.0 Hz, 2H), 3.75 (s, 3H), 3.55 (s, 2H), 3.27-3.20 (m, 2H), 3.01-2.84 (m, 4H), 2.67 (t, J=11.0 Hz, 2H), 2.62-2.52 (m, 6H), 2.44-2.24 (m, 8H), 1.96 (t, J=11.1 Hz, 7H), 1.86 (d, J=10.9 Hz, 2H), 1.59-1.48 (m, 2H), 1.07 (d, J=6.3 Hz, 4H), 0.78 (s, 3H). [M+H]⁺=1044.4

### Example 427: 3-(4-(2-(4-(1-(4-((5-bromo-4-((2-cyclopropyl-5-(dimethylphosphoryl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

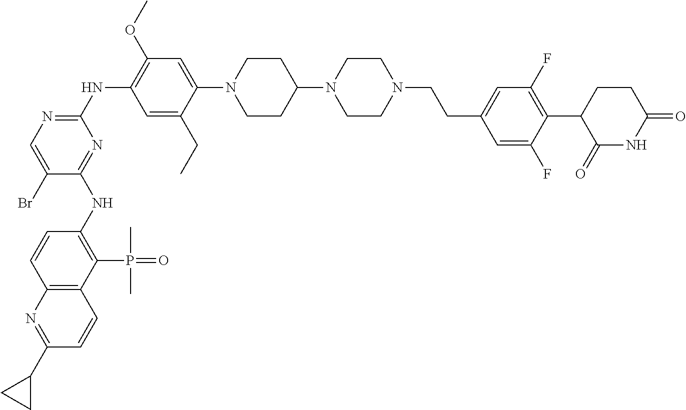 [SMILES: CCc1cc(Nc2ncc(Br)c(Nc3ccc4nc(C5CC5)ccc4c3P(C)(C)=O)n2)c(OC)cc1N1CCC(N2CCN(CCc3cc(F)c(C4CCC(=O)NC4=O)c(F)c3)CC2)CC1]

The titled compound was prepared in a manner similar to that in Example 426. ¹H NMR (500 MHz, DMSO) δ 11.75 (s, 1H), 10.95 (s, 1H), 8.49 (d, J=8.9 Hz, 1H), 8.22 (d, J=24.3 Hz, 2H), 8.03 (s, 1H), 7.74 (d, J=9.2 Hz, 1H), 7.44 (d, J=8.9 Hz, 1H), 7.28 (s, 1H), 7.03 (d, J=10.0 Hz, 2H), 6.75 (s, 1H), 4.20 (dd, J=12.6, 5.1 Hz, 1H), 3.75 (s, 3H), 3.28-3.21 (m, 2H), 2.95 (d, J=11.0 Hz, 2H), 2.86-2.73 (m, 3H), 2.67 (t, J=11.0 Hz, 2H), 2.53 (d, J=6.6 Hz, 5H), 2.49-2.39 (m, 4H), 2.28 (dd, J=12.7, 6.3 Hz, 4H), 2.18-2.07 (m, 1H), 2.04-1.92 (m, 7H), 1.86 (d, J=11.0 Hz, 2H), 1.55 (dd, J=20.2, 11.3 Hz, 2H), 1.06 (d, J=6.4 Hz, 4H), 0.78 (s, 3H). [M+H]⁺=984.3.

### Example 429: 3-(4-(2-(4-(1-(4-((5-bromo-4-((5-(dimethylphosphoryl)-2-methyl-1-oxo-1,2-dihydroisoquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: 6-bromo-2-methylisoquinolin-1(2H)-one

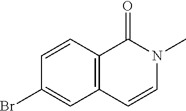

To a stirred mixture of 6-bromoisoquinolin-1(2H)-one (5.00 g, 22.3 mmol), tetrabutylammonium bromide (1.08 g, 3.35 mmol) and iodomethane (1.67 mL, 26.8 mmol) in toluene (300 mL), was added aqueous NaOH solution (50 wt %, 50 mL). The resulting mixture was stirred vigorously at ambient temperature. After 14 hrs, the mixture was diluted with ethyl ether (500 mL) and washed with water, saturated NaHCO₃, and brine in turn. The organic phase was dried over anhydrous Na₂SO₄ and concentrated in vacuum to give product (4.20 g, 79.1%). [M+H]⁺=238.1.

### Step 2: 6-amino-2-methylisoquinolin-1(2H)-one

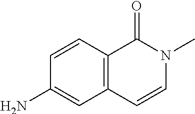

A high-pressure reactor equipped with a magnetic stirrer, was charged with 6-bromo-2-methylisoquinolin-1(2H)-one (4.10 g, 17.2 mmol), copper (powder, 1.31 g, 20.7 mmol), ammonium hydroxide (10 mL) and isopropanol (10 mL). The resulting suspension was stirred at 100° C. for 21 hrs. After cooling to room temperature, the mixture was concentrated in vacuum and purified by column chromatography (0-5% MeOH in DCM) to afford product (2.50 g, 83.4%). [M+H]⁺=175.2.

### Step 3: 6-amino-5-iodo-2-methylisoquinolin-1(2H)-one

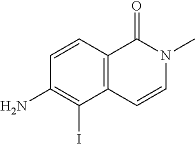

To a stirred solution of 6-amino-2-methylisoquinolin-1(2H)-one (2.40 g, 13.8 mmol) in AcOH (40 mL), was added a solution of ICl (2.70 g, 16.6 mmol) in AcOH (10 mL) dropwise at room temperature. After being stirred 2 hrs, the reaction mixture was concentrated in vacuum to afford the residue which was purified by column chromatography (DCM/MeOH) to afford product (3.00 g, 72.4%). [M+H]⁺=301.2.

### Step 4: 6-amino-5-(dimethylphosphoryl)-2-methylisoquinolin-1(2H)-one

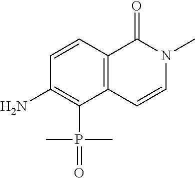 [SMILES: Cn1ccc2c(P(C)(C)=O)c(N)ccc2c1=O]

A mixture of 6-amino-5-iodo-2-methylisoquinolin-1(2H)-one (2.20 g, 7.33 mmol), dimethylphosphine oxide (859 mg, 11.00 mmol), K₃PO₄ (3.11 g, 14.7 mmol), XANTPHOS (424 mg, 0.73 mmol), Pd(OAc)₂ (165 mg, 0.73 mmol), DMF (25 mL) and water (5 mL) was stirred at room temperature. The suspension was degassed under vacuum and purged with N₂ three times, then stirred at 120° C. for 1 hr. After cooling to room temperature, the mixture was concentrated in vacuum, the residue was purified by column chromatography (0-10% MeOH in DCM) to afford the product (1.45 g, 79.1%). [M+H]⁺=251.2.

### Step 5: 6-((5-bromo-2-chloropyrimidin-4-yl)amino)-5-(dimethylphosphoryl)-2-methylisoquinolin-1(2H)-one

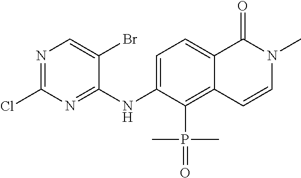 [SMILES: Cn1ccc2c(P(C)(C)=O)c(Nc3nc(Cl)ncc3Br)ccc2c1=O]

To a stirred solution of 6-amino-5-(dimethylphosphoryl)-2-methylisoquinolin-1(2H)-one (300 mg, 1.2 mmol) and 5-bromo-2,4-dichloropyrimidine (542 mg, 2.4 mmol) in DMF (10 mL) was added NaH (96 mg, 2.4 mmol) at 0° C. The resulting mixture was stirred at rt for 2 hours. The reaction was extracted with DCM (2×10.0 mL). The combined organic layer was washed with brine (2×10.0 mL), dried over Na2SO4 and concentrated under vacuum to afford the crude residue, which was purified with silica gel column chromatography (DCM:MeOH=10:1) to give the title product (300 mg, 57%). [M+H]⁺=441.6.

### Step 6: 6-((5-bromo-2-((5-ethyl-2-methoxy-4-(4-(piperazin-1-yl)piperidin-1-yl)phenyl)amino)pyrimidin-4-yl)amino)-5-(dimethylphosphoryl)-2-methylisoquinolin-1(2H)-one

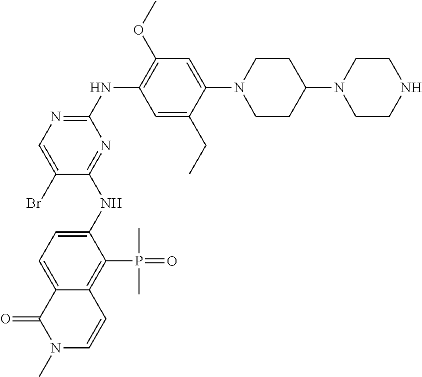 [SMILES: CCc1cc(Nc2ncc(Br)c(Nc3ccc4c(=O)n(C)ccc4c3P(C)(C)=O)n2)c(OC)cc1N1CCC(N2CCNCC2)CC1]

To a stirred solution of 6-((5-bromo-2-chloropyrimidin-4-yl)amino)-5-(dimethylphosphoryl)-2-methylisoquinolin-1(2H)-one (300 mg 0.57 mmol) and tert-butyl 4-(1-(4-amino-2-ethyl-5-methoxyphenyl)piperidin-4-yl)piperazine-1-carboxylate (286 mg, 0.68 mmol) in t-BuOH (10 mL) was added MsOH (218 mg, 2.28 mmol). The resulting mixture was stirred at 95° C. for 16 hours. The reaction was extracted with DCM (2×20.0 mL). The combined organic layer was washed with brine (2×20.0 mL), dried over Na₂SO₄ and concentrated under vacuum to afford the crude residue, which was purified with silica gel column chromatography (DCM: MeOH=8:1) to give the title product (300 mg, 73%). [M+H]⁺=723.6.

### Step 7: 3-(4-(2-(4-(1-(4-((5-bromo-4-((5-(dimethylphosphoryl)-2-methyl-1-oxo-1,2-dihydroisoquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

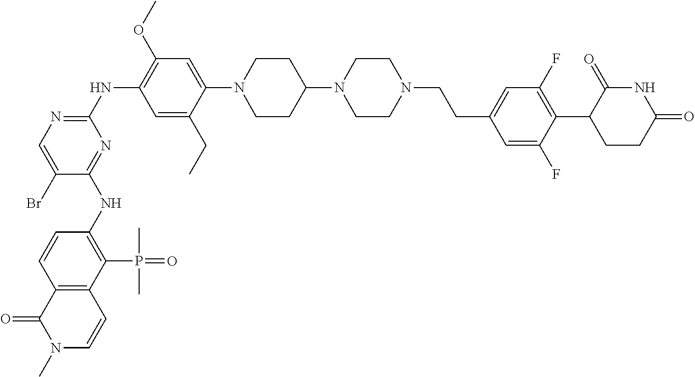 [SMILES: CCc1cc(Nc2ncc(Br)c(Nc3ccc4c(=O)n(C)ccc4c3P(C)(C)=O)n2)c(OC)cc1N1CCC(N2CCN(CCc3cc(F)c(C4CCC(=O)NC4=O)c(F)c3)CC2)CC1]

The titled compound was synthesized in the procedures similar to Example 200; ¹H NMR (500 MHz, DMSO) δ 12.43 (s, 1H), 10.95 (s, 1H), 8.32 (s, 1H), 8.22 (d, J=4.4 Hz, 1H), 8.16 (dd, J=14.2, 8.9 Hz, 2H), 7.46 (d, J=7.9 Hz, 1H), 7.38 (s, 1H), 7.03 (d, J=10.0 Hz, 2H), 6.76 (s, 1H), 6.63 (d, J=7.8 Hz, 1H), 4.20 (dd, J=12.6, 5.0 Hz, 1H), 3.75 (s, 3H), 3.50 (s, 3H), 3.01 (d, J=10.9 Hz, 2H), 2.83-2.65 (m, 6H), 2.54 (t, J=7.5 Hz, 7H), 2.49-2.43 (m, 4H), 2.38-2.30 (m, 1H), 2.14-2.10 (m, 1H), 1.97 (dd, J=13.2, 7.9 Hz, 8H), 1.86 (d, J=10.7 Hz, 2H), 1.63-1.52 (m, 2H), 0.96 (t, J=7.3 Hz, 3H); [M+H]⁺=974.4.

### Example 431: 3-(4-(2-(4-(1-(4-((5-bromo-4-((5-(dimethylphosphoryl)-2-methyl-1-oxo-1,2-dihydrophthalazin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: 6-bromo-2-methylphthalazin-1(2H)-one

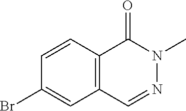

To a stirred mixture of 6-bromophthalazin-1(2H)-one (5 g, 22.2 mmol) and NaH (1.1 g, 60% wt, 27.5 mmol) in DMF (50 mL), was added methyl iodide (2.1 mL, 33.3 mmol) at 0° C. under N₂ atmosphere. The resulting mixture was stirred for 3 days at room temperature under nitrogen atmosphere. The reaction was quenched with sat. aq. NH₄Cl solution and extracted with EA. The combined organic layers were washed with brine, dried over anhydrous Na₂SO₄. After filtration, the filtrate was concentrated under reduced pressure. The residue was purified by column chromatography (DCM/MeOH, 0-5%) to afford product (4.6 g, 86.6%). [M+H]⁺=239.1.

### Step 2: 6-amino-2-methylphthalazin-1(2H)-one

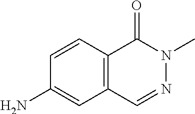

A high pressure reactor equipped with a magnetic stirrer, was charged with 6-bromo-2-methylphthalazin-1(2H)-one (4.50 g, 18.8 mmol), copper (powder, 1.20 g, 18.8 mmol) and NH₄OH (11.0 m1, 312.5 mmol) in isopropanol (20 mL). The resulting suspension was stirred overnight at 100° C. After cooling to room temperature, the mixture was concentrated in vacuum and purified by column chromatography (DCM/MeOH, 0-5%) to afford the product (2.3 g, 69.7%). [M+H]⁺=176.2.

### Step 3: 6-amino-5-iodo-2-methylphthalazin-1(2H)-one

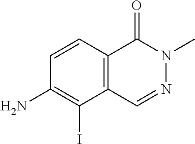

To a stirred solution of 6-amino-2-methylphthalazin-1(2H)-one (2.20 g, 12.6 mmol) in AcOH (40 mL), was added a solution of iodine monochloride (2.45 g, 15.1 mmol) in AcOH (10 mL) dropwise at room temperature.

The reaction mixture was stirred at rt for 2 h and concentrated in vacuum to afford the residue which was purified by column chromatography (DCM/MeOH, 0-5%) to provide product (3.00 g, 72.4%) [M+H]⁺=302.2.

### Step 4: 6-amino-5-(dimethylphosphoryl)-2-methylphthalazin-1(2H)-one

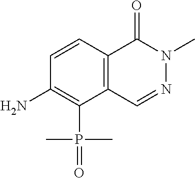 [SMILES: Cn1ncc2c(P(C)(C)=O)c(N)ccc2c1=O]

A mixture of 6-amino-5-iodo-2-methylphthalazin-1(2H)-one (2.0 g, 6.6 mmol), dimethylphosphineoxide (622.1 mg, 7.9 mmol), K₃PO₄ (2.1 g, 9.9 mmol), Pd(OAc)₂ (149.1 mg, 0.6 mmol) and XantPhos (384.3 mg, 0.6 mmol) in DMF (20 mL) and H₂O (4 mL) was stirred for 2 h at 120° C. under nitrogen atmosphere. After cooling to room temperature, the mixture was concentrated in vacuum, the residue was purified by column chromatography (DCM/MeOH, 0-10%) to afford the product (1.07 g, 64.12%). [M+H]⁺=252.2.

### Step 4: 6-((5-bromo-2-chloropyrimidin-4-yl)amino)-5-(dimethylphosphoryl)-2-methylphthalazin-1(2H)-one

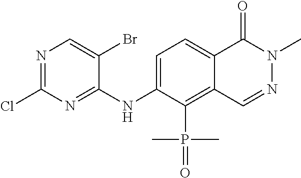 [SMILES: Cn1ncc2c(P(C)(C)=O)c(Nc3nc(Cl)ncc3Br)ccc2c1=O]

To a stirred solution of 6-amino-5-(dimethylphosphoryl)-2-methylphthalazin-1(2H)-one (500 mg, 2.0 mmol) and 5-bromo-2,4-dichloropyrimidine (912 mg, 4.0 mmol) in DMF (10 mL) was added NaH (96 mg, 2.4 mmol) at 0° C. The resulting mixture was stirred at rt for 2 hours. The reaction was extracted with DCM (2×10.0 mL). The combined organic layer was washed with brine (2×10.0 mL), dried over Na2SO4 and concentrated under vacuum to afford the crude residue, which was purified with silica gel column chromatography (DCM: MeOH=10:1) to give the product (600 mg, 68%). [M+H]⁺=442.6.

### Step 5: 6-((5-bromo-2-((5-ethyl-2-methoxy-4-(4-(piperazin-1-yl)piperidin-1-yl)phenyl)amino)pyrimidin-4-yl)amino)-5-(dimethylphosphoryl)-2-methylphthalazin-1(2H)-one

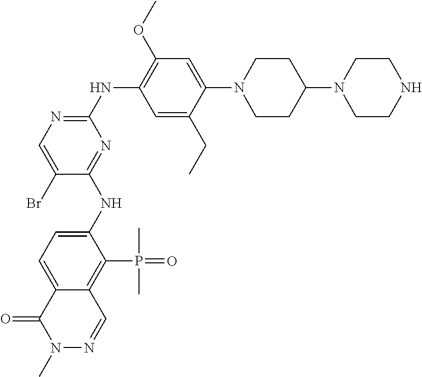 [SMILES: CCc1cc(Nc2ncc(Br)c(Nc3ccc4c(=O)n(C)ncc4c3P(C)(C)=O)n2)c(OC)cc1N1CCC(N2CCNCC2)CC1]

To a stirred solution of 6-((5-bromo-2-chloropyrimidin-4-yl)amino)-5-(dimethylphosphoryl)-2-methylphthalazin-1(2H)-one (100 mg, 0.23 mmol) and tert-butyl 4-(1-(4-amino-2-ethyl-5-methoxyphenyl)piperidin-4-yl)piperazine-1-carboxylate (147 mg, 0.35 mmol) in t-BuOH (10 mL) was added MsOH (88 mg, 0.92 mmol). The resulting mixture was stirred at 95° C. for 16 hours. Water (20 mL) was poured into the mixture. Then the mixture was adjusted to pH=8 with sat. aq. NaHCO₃ solution and extracted with DCM (2×20.0 mL). The combined organic layer was washed with brine (2×20.0 mL), dried over Na₂SO₄ and concentrated under vacuum to afford the crude residue, which was purified with silica gel column chromatography (DCM: MeOH=8:1) to give the title product (100 mg, 63%). [M+H]⁺=724.6.

### Step 6: 3-(4-(2-(4-(1-(4-((5-bromo-4-((5-(dimethylphosphoryl)-2-methyl-1-oxo-1,2-dihydrophthalazin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

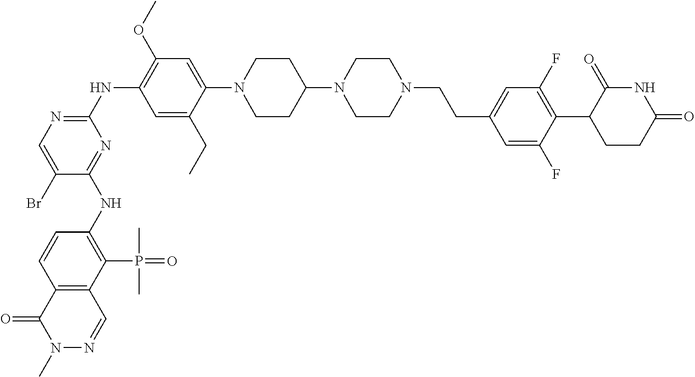 [SMILES: CCc1cc(Nc2ncc(Br)c(Nc3ccc4c(=O)n(C)ncc4c3P(C)(C)=O)n2)c(OC)cc1N1CCC(N2CCN(CCc3cc(F)c(C4CCC(=O)NC4=O)c(F)c3)CC2)CC1]

The titled compound (8 mg, 30%) was prepared in a manner similar to that in Example 204 step 6 from 6-((5-bromo-2-((5-ethyl-2-methoxy-4-(4-(piperazin-1-yl)piperidin-1-yl)phenyl)amino)pyrimidin-4-yl)amino)-5-(dimethylphosphoryl)-2-methylphthalazin-1(2H)-one and 2-(4-(2,6-dioxopiperidin-3-yl)-3,5-difluorophenyl)acetaldehyde. ¹H NMR (500 MHz, DMSO) δ 12.21 (s, 1H), 10.94 (s, 1H), 8.57 (s, 1H), 8.20 (t, J=18.3 Hz, 3H), 7.31 (s, 1H), 7.03 (d, J=10.0 Hz, 2H), 6.75 (s, 1H), 4.23-4.17 (m, 1H), 3.73 (d, J=16.1 Hz, 6H), 2.98 (s, 2H), 2.85-2.62 (m, 7H), 2.54 (d, J=7.3 Hz, 8H), 2.33 (d, J=27.0 Hz, 4H), 2.02 (d, J=12.2 Hz, 9H), 1.85 (d, J=11.0 Hz, 2H), 1.56 (d, J=10.0 Hz, 2H), 0.92 (s, 3H); [M+H]⁺=975.4.

### Example 428: 3-(4-(2-(4-(1-(4-((5-bromo-4-((5-(dimethylphosphoryl)-3-methyl-4-oxo-3,4-dihydroquinazolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: 3-methyl-6-nitroquinazolin-4-one

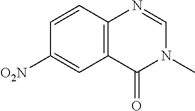

Into a 250 mL round-bottom flask were added 6-nitro-3H-quinazolin-4-one (10.0 g, 52.31 mmol) and Cs₂CO₃ (25.57 g, 78.44 mmol) in dimethylformamide (150 mL) at room temperature. CH₃I (11.14 g, 78.45 mmol) was added in portions at 0° C. under nitrogen atmosphere. The resulting mixture was stirred for 3 h at room temperature under nitrogen atmosphere. The resulting mixture was extracted with EtOAc (350 mL). The combined organic layers were washed with brine (100 mL), dried over anhydrous Na₂SO₄. After filtration, the filtrate was concentrated under reduced pressure. The residue was purified by silica gel column chromatography, eluted with PE/EtOAc (1:1) to afford the product (9 g, 83.85%). [M+H]⁺=206.2.

### Step 2: 6-amino-3-methylquinazolin-4-one

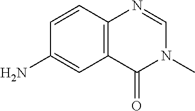

Into a 250 mL round-bottom flask were added 3-methyl-6-nitroquinazolin-4-one (5.0 g, 24.37 mmol) and Pd/C (3.0 g, 10% wt) in MeOH (150 mL) at room temperature. The resulting mixture was stirred for 2 h at room temperature under hydrogen atmosphere. The resulting mixture was diluted with MeOH (100 mL). The resulting mixture was concentrated under vacuum. The residue was purified by silica gel column chromatography, eluted with PE/EtOAc (1:1) to afford the product (3.2 g, 74.95%). [M+H]⁺=176.2.

### Step 3: 6-amino-5-iodo-3-methylquinazolin-4-one

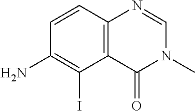

Into a 250 mL round-bottom flask were added 6-amino-3-methylquinazolin-4-one (3.0 g, 17.12 mmol) and AcOH (50 mL) at room temperature. ICl (3.06 g, 18.88 mmol) was added in portions at 0° C. The resulting mixture was stirred for 2 h at room temperature under air atmosphere. The resulting mixture was extracted with EtOAc (250 mL). The combined organic layers were washed with brine (120 mL), dried over anhydrous Na₂SO₄. After filtration, the filtrate was concentrated under reduced pressure. The residue was purified by silica gel column chromatography, eluted with PE/EtOAc (1:1) to afford the product (1.5 g, 29.09%). [M+H]⁺=302.2.

### Step 4: 6-amino-5-(dimethylphosphoryl)-3-methylquinazolin-4-one

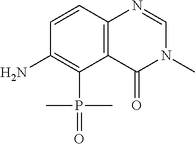 [SMILES: Cn1cnc2ccc(N)c(P(C)(C)=O)c2c1=O]

A mixture of 6-amino-5-iodo-3-methylquinazolin-4-one (1.5 g, 4.98 mmol), dimethylphosphine oxide (0.58 g, 7.44 mmol), Pd(OAc)₂ (0.11 g, 0.49 mmol), XantPhos (0.29 g, 0.50 mmol), K₃PO₄ (1.59 g, 7.54 mmol) in DMF (15 mL) and H₂O (3.0 mL) was stirred at room temperature under air atmosphere. The resulting mixture was stirred for 3 h at 120° C. under nitrogen atmosphere. The resulting mixture was extracted with EtOAc (200 mL). The combined organic layers were washed with brine (100 mL), dried over anhydrous Na₂SO₄. After filtration, the filtrate was concentrated under reduced pressure. The residue was purified by Prep-TLC PE/EtOAc (1:1) to afford the product (659.8 mg, 52.72%). [M+H]⁺=252.2.

### Step 5: 6-((5-bromo-2-chloropyrimidin-4-yl)amino)-5-(dimethylphosphoryl)-3-methylquinazolin-4(3H)-one

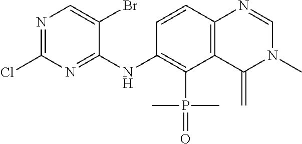 [SMILES: C=C1c2c(ccc(Nc3nc(Cl)ncc3Br)c2P(C)(C)=O)N=CN1C]

The titled compound (450 mg, 89%) was prepared in a manner similar to that in Example 431 step 4 from 6-amino-5-(dimethylphosphoryl)-3-methylquinazolin-4-one and 5-bromo-2,4-dichloropyrimidine. [M+H]+=442.0.

### Step 6: 6-((5-bromo-2-((5-ethyl-2-methoxy-4-(4-(piperazin-1-yl)piperidin-1-yl)phenyl)amino)pyrimidin-4-yl)amino)-5-(dimethylphosphoryl)-3-methylquinazolin-4(3H)-one

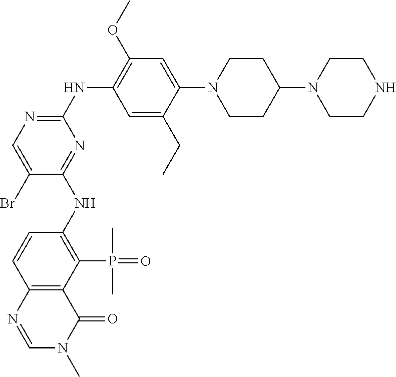 [SMILES: CCc1cc(Nc2ncc(Br)c(Nc3ccc4ncn(C)c(=O)c4c3P(C)(C)=O)n2)c(OC)cc1N1CCC(N2CCNCC2)CC1]

The titled compound (230 mg, 45%) was prepared in a manner similar to that in Example 431 step 5 from 6-((5-bromo-2-chloropyrimidin-4-yl)amino)-5-(dimethylphosphoryl)-3-methylquinazolin-4(3H)-one and tert-butyl 4-(1-(4-amino-2-ethyl-5-methoxyphenyl)piperidin-4-yl)piperazine-1-carboxylate. [M+H]⁺=724.2

### Step 7: 3-(4-(2-(4-(1-(4-((5-bromo-4-((5-(dimethylphosphoryl)-3-methyl-4-oxo-3,4-dihydroquinazolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

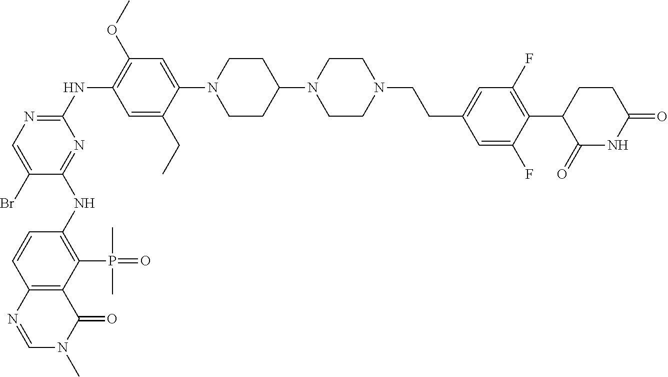 [SMILES: CCc1cc(Nc2ncc(Br)c(Nc3ccc4ncn(C)c(=O)c4c3P(C)(C)=O)n2)c(OC)cc1N1CCC(N2CCN(CCc3cc(F)c(C4CCC(=O)NC4=O)c(F)c3)CC2)CC1]

The titled compound (11 mg, 16%) was prepared in a manner similar to that in Example 204 step 6 from 6-((5-bromo-2-((5-ethyl-2-methoxy-4-(4-(piperazin-1-yl)piperidin-1-yl)phenyl)amino)pyrimidin-4-yl)amino)-5-(dimethylphosphoryl)-3-methylquinazolin-4(3H)-one and 2-(4-(2,6-dioxopiperidin-3-yl)-3,5-difluorophenyl)acetaldehyde. ¹H NMR (500 MHz, DMSO) δ 13.04 (s, 1H), 10.95 (s, 1H), 8.46 (d, J=6.7 Hz, 1H), 8.39 (s, 1H), 8.20 (d, J=1.1 Hz, 1H), 7.96 (s, 1H), 7.62 (d, J=9.0 Hz, 1H), 7.43 (s, 1H), 7.03 (d, J=10.3 Hz, 2H), 6.75 (s, 1H), 4.20 (dd, J=12.6, 4.8 Hz, 1H), 3.76 (s, 3H), 3.49 (s, 3H), 3.29 (s, 2H), 2.97 (d, J=11.3 Hz, 2H), 2.87-2.74 (m, 3H), 2.64 (dd, J=41.0, 29.5 Hz, 8H), 2.41 (dd, J=24.2, 16.9 Hz, 6H), 2.13 (dt, J=22.6, 11.2 Hz, 1H), 2.00 (d, J=14.5 Hz, 7H), 1.86 (d, J=9.7 Hz, 2H), 1.56 (d, J=10.1 Hz, 2H), 0.87 (s, 3H). [M+H]⁺=975.3.

### Example 430: 3-(4-(2-(4-(1-(4-((5-bromo-4-((5-(dimethylphosphoryl)quinoxalin-6-yl)amino)pyrimidin-2-yl)amino)-2-(1-hydroxyethyl)-5-methoxyphenyl)piperidin-4-yl)piperazin-1-yl)ethyl)-2,6-difluorophenyl)piperidine-2,6-dione

### Step 1: tert-butyl 4-(1-(2-acetyl-5-methoxy-4-nitrophenyl)piperidin-4-yl)piperazine-1-carboxylate

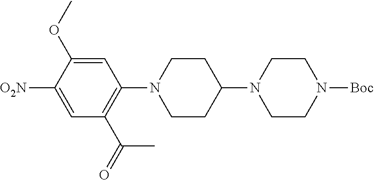 [SMILES: COc1cc(N2CCC(N3CCN(C(=O)OC(C)(C)C)CC3)CC2)c(C(C)=O)cc1[N+](=O)[O-]]

To a stirred solution of 1-(2-fluoro-4-methoxy-5-nitrophenyl)ethan-1-one (500 mg, 2.34 mmol) and tert-butyl 4-(piperidin-4-yl)piperazine-1-carboxylate (944 mg, 3.51 mmol) in DMF (15 mL) was added K₂CO₃ (646 mg, 4.68 mmol). The resulting mixture was stirred at 100° C. for 16 hours. The reaction was concentrated under vacuum to afford the crude residue, which was purified with silica gel column chromatography (DCM: MeOH=15:1) to give the title product (800 mg, 74%). [M+H]⁺=463.3.

### Step 2: tert-butyl 4-(1-(2-acetyl-4-amino-5-methoxyphenyl)piperidin-4-yl)piperazine-1-carboxylate

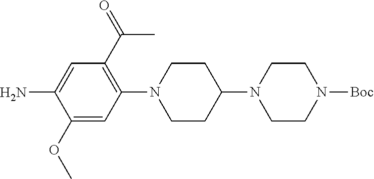 [SMILES: COc1cc(N2CCC(N3CCN(C(=O)OC(C)(C)C)CC3)CC2)c(C(C)=O)cc1N]

To a stirred solution of tert-butyl 4-(1-(2-acetyl-5-methoxy-4-nitrophenyl)piperidin-4-yl)piperazine-1-carboxylate (800 mg, 1.73 mmol) in THF (15 mL) and H₂O (15 mL) was added NH₄Cl (366 mg, 6.92 mmol). The resulting mixture was stirred at rt for 2 hours. The reaction was extracted with DCM (2×20.0 mL). The combined organic layer was washed with brine (2×20.0 mL), dried over Na₂SO₄ and concentrated under vacuum to afford the crude residue, which was purified with silica gel column chromatography (DCM: MeOH=10:1) to give the title product (600 mg, 80%). [M+H]⁺=433.3.

### Step 3: 1-(5-((5-bromo-4-((5-(dimethylphosphoryl)quinoxalin-6-yl)amino)pyrimidin-2-yl)amino)-4-methoxy-2-(4-(piperazin-1-yl)piperidin-1-yl)phenyl)ethan-1-one

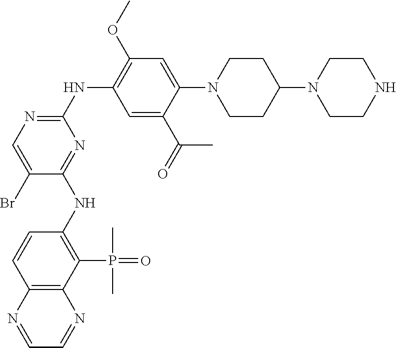 [SMILES: COc1cc(N2CCC(N3CCNCC3)CC2)c(C(C)=O)cc1Nc1ncc(Br)c(Nc2ccc3nccnc3c2P(C)(C)=O)n1]

The titled compound (510 mg, 60%) was prepared in a manner similar to that in Example 200 step 14 from (6-((5-bromo-2-chloropyrimidin-4-yl)amino)quinoxalin-5-yl)dimethylphosphine oxide and tert-butyl 4-(1-(2-acetyl-4-amino-5-methoxyphenyl)piperidin-4-yl)piperazine-1-carboxylate. [M+H]⁺=708.4.

### Step 4: (6-((5-bromo-2-((5-(1-hydroxyethyl)-2-methoxy-4-(4-(piperazin-1-yl)piperidin-1-yl)phenyl)amino)pyrimidin-4-yl)amino)luinoxalin-5-yl)dimethylphosphine oxide

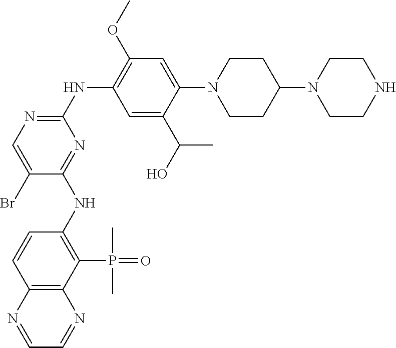 [SMILES: COc1cc(N2CCC(N3CCNCC3)CC2)c(C(C)O)cc1Nc1ncc(Br)c(Nc2ccc3nccnc3c2P(C)(C)=O)n1]

To a stirred solution of 1-(5-((5-bromo-4-((5-(dimethylphosphoryl)quinoxalin-6-yl)amino)pyrimidin-2-yl)amino)-4-methoxy-2-(4-(piperazin-1-yl)piperidin-1-yl)phenyl)ethan-1-one (500 mg, 0.7 mmol) in MeOH (15 mL) was added NaBH₄ (53 mg, 1.4 mmol). The resulting mixture was stirred at rt for 16 hours. The reaction was extracted with DCM (2×20.0 mL). The combined organic layer was washed with brine (2×20.0 mL), dried over Na₂SO₄ and concentrated under vacuum to afford the title product (400 mg, 80%). [M+H]⁺=710.4.

### Step 5: 3-(4-(2-(4-(1-(4-((5-bromo-4-((5-(dimethylphosphoryl)quinoxalin-6-yl)amino)pyrimidin-2-yl)amino)-2-(1-hydroxyethyl)-5-methoxyphenyl)piperidin-4-yl)piperazin-1-yl)ethyl)-2,6-difluorophenyl)piperidine-2,6-dione

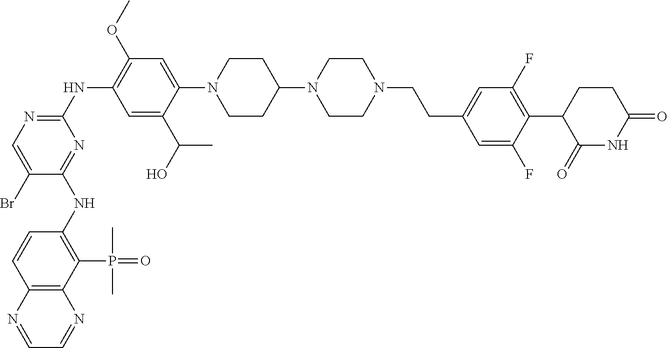 [SMILES: COc1cc(N2CCC(N3CCN(CCc4cc(F)c(C5CCC(=O)NC5=O)c(F)c4)CC3)CC2)c(C(C)O)cc1Nc1ncc(Br)c(Nc2ccc3nccnc3c2P(C)(C)=O)n1]

The titled compound (5 mg, 12%) was prepared in a manner similar to that in Example 204 step 6 from (6-((5-bromo-2-((5-(1-hydroxyethyl)-2-methoxy-4-(4-(piperazin-1-yl)piperidin-1-yl)phenyl)amino)pyrimidin-4-yl)amino)quinoxalin-5-yl)dimethylphosphine oxide and 2-(4-(2,6-dioxopiperidin-3-yl)-3,5-difluorophenyl)acetaldehyde. ¹H NMR (500 MHz, DMSO) δ 12.68 (s, 1H), 10.95 (s, 1H), 8.84 (dd, J=7.7, 1.8 Hz, 3H), 8.47 (s, 1H), 8.26 (d, J=9.1 Hz, 1H), 7.93 (d, J=9.2 Hz, 1H), 7.44 (s, 1H), 7.03 (d, J=10.0 Hz, 2H), 6.87 (s, 1H), 5.03 (dt, J=18.3, 9.0 Hz, 2H), 4.20 (dd, J=12.7, 5.0 Hz, 1H), 3.76 (s, 3H), 3.14 (d, J=10.6 Hz, 1H), 2.96 (d, J=10.2 Hz, 1H), 2.86-2.63 (m, 6H), 2.59-2.51 (m, 8H), 2.48-2.42 (m, 2H), 2.33 (t, J=11.2 Hz, 1H), 2.13 (dd, J=13.1, 3.8 Hz, 1H), 2.01 (d, J=14.1 Hz, 7H), 1.87 (t, J=12.5 Hz, 2H), 1.57 (dd, J=7.5, 4.1 Hz, 2H), 1.21 (d, J=6.2 Hz, 3H); [M+H]⁺=961.4.

### Example 432: 3-(4-((2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)-2-oxoethyl)amino)-2,6-difluorophenyl)piperidine-2,6-dione

### Step 1: methyl 2-((4-(2,6-bis(benzyloxy)pyridin-3-yl)-3,5-difluorophenyl)amino)acetate

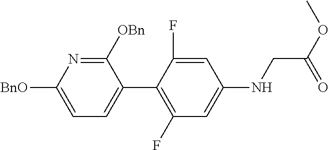 [SMILES: COC(=O)CNc1cc(F)c(-c2ccc(OCc3ccccc3)nc2OCc2ccccc2)c(F)c1]

To a stirred solution of 2,6-bis(benzyloxy)-3-(4-bromo-2,6-difluorophenyl)pyridine (500 mg, 1.03 mmol) and methyl 2-aminoacetate (138 mg, 1.6 mmol) in toluene (20 mL) was added Ruphos G3 (85 mg, 0.1 mmol) and Cs₂CO₃ (669 mg, 2.06 mmol). The resulting mixture was stirred at 100° C. for 16 hours under N₂ atmosphere. The reaction was concentrated under vacuum to afford the crude product, which was purified with silica gel column chromatography (PE:EA=3:1) to give the title product (400 mg, 79%). [M+H]⁺=491.3.

### Step 2: 2-((4-(2,6-bis(benzyloxy)pyridin-3-yl)-3,5-difluorophenyl)amino)acetic acid

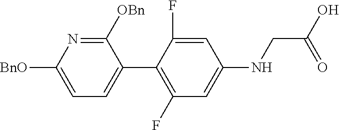 [SMILES: O=C(O)CNc1cc(F)c(-c2ccc(OCc3ccccc3)nc2OCc2ccccc2)c(F)c1]

To a stirred solution of methyl 2-((4-(2,6-bis(benzyloxy)pyridin-3-yl)-3,5-difluorophenyl)amino)acetate (400 mg, 0.82 mmol) in THF (5 mL), MeOH (5 mL) and H₂O (5 mL) was added LiOH (43 mg, 1.64 mmol).

The resulting mixture was stirred at rt for 1 hour. The reaction was extracted with EA (2×20.0 mL). The combined organic layer was washed with brine (2×20.0 mL), dried over Na₂SO₄ and concentrated under vacuum to afford the title product (300 mg, 76%). [M+H]⁺=477.4.

### Step 3: 2-((4-(2,6-dioxopiperidin-3-yl)-3,5-difluorophenyl)amino)acetic acid

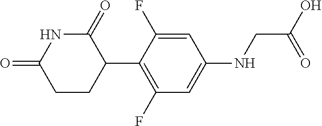 [SMILES: O=C(O)CNc1cc(F)c(C2CCC(=O)NC2=O)c(F)c1]

To a stirred solution of 2-((4-(2,6-bis(benzyloxy)pyridin-3-yl)-3,5-difluorophenyl)amino)acetic acid (300 mg, 0.63 mmol) in MeOH (15 mL) was added Pd/C (60 mg). The resulting mixture was stirred at rt for 16 hour under H₂ atmosphere. The reaction was filtrated, the filtration was concentrated under vacuum to afford the title product (150 mg, 80%). [M+H]⁺=299.2.

### Step 4: 3-(4-((2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)-2-oxoethyl)amino)-2,6-difluorophenyl)piperidine-2,6-dione

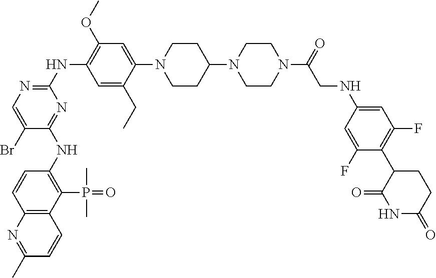 [SMILES: CCc1cc(Nc2ncc(Br)c(Nc3ccc4nc(C)ccc4c3P(C)(C)=O)n2)c(OC)cc1N1CCC(N2CCN(C(=O)CNc3cc(F)c(C4CCC(=O)NC4=O)c(F)c3)CC2)CC1]

The titled compound (12 mg, 24%) was prepared in a manner similar to that in Example 208 step 8 from (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide and 2-((4-(2,6-dioxopiperidin-3-yl)-3,5-difluorophenyl)amino)acetic acid. ¹H NMR (500 MHz, DMSO) δ 11.76 (s, 1H), 10.83 (s, 1H), 8.56 (d, J=8.9 Hz, 1H), 8.30 (s, 1H), 8.21 (s, 1H), 7.98 (s, 1H), 7.87 (d, J=9.2 Hz, 1H), 7.42 (d, J=8.9 Hz, 1H), 7.38 (s, 1H), 6.74 (s, 1H), 6.41 (d, J=12.0 Hz, 2H), 6.23 (d, J=4.5 Hz, 1H), 3.99 (dd, J=12.6, 5.0 Hz, 1H), 3.93 (d, J=4.7 Hz, 2H), 3.76 (s, 3H), 3.49 (s, 3H), 2.95 (d, J=11.0 Hz, 2H), 2.82-2.73 (m, 1H), 2.69 (s, 6H), 2.57 (s, 2H), 2.45-2.35 (m, 2H), 2.30 (d, J=6.7 Hz, 2H), 2.12-2.02 (m, 1H), 1.98 (d, J=13.3 Hz, 7H), 1.83 (d, J=10.0 Hz, 2H), 1.59-1.56 (m, 2H), 0.77 (s, 3H); [M+H]⁺=987.7.

### Example 433: 3-(4-((2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)(methyl)amino)-2,6-difluorophenyl)piperidine-2,6-dione

### Step 1: 2-((4-(2,6-bis(benzyloxy)pyridin-3-yl)-3 ,5-difluorophenyl)(methyl)amino)ethanol

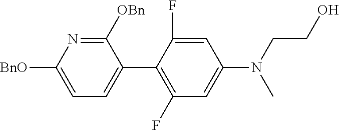 [SMILES: CN(CCO)c1cc(F)c(-c2ccc(OCc3ccccc3)nc2OCc2ccccc2)c(F)c1]

The titled compound (500 mg, 80%) was prepared in a manner similar to that in Example 432 step 1 from 2,6-bis(benzyloxy)-3-(4-bromo-2,6-difluorophenyl)pyridineand 2-(methylamino)ethan-1-ol. [M+H]⁺=477.7.

### Step 2: 3-(2,6-difluoro-4-((2-hydroxyethyl)(methyl)amino)phenyl)piperidine-2,6-dione

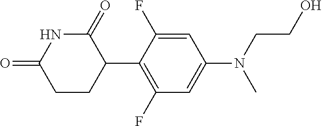 [SMILES: CN(CCO)c1cc(F)c(C2CCC(=O)NC2=O)c(F)c1]

The titled compound (250 mg, 80%) was prepared in a manner similar to that in Example 432 step 3 from 2-((4-(2,6-bis(benzyloxy)pyridin-3-yl)-3,5-difluorophenyl)(methyl)amino)ethanol and Pd/C. [M+H]⁺=299.3.

### Step 3: 2-((4-(2,6-dioxopiperidin-3-yl)-3 ,5-difluorophenyl)(methyl)amino)ethyl methanesulfonate

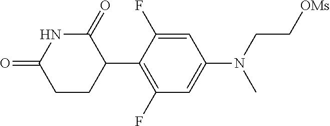 [SMILES: CN(CCOS(C)(=O)=O)c1cc(F)c(C2CCC(=O)NC2=O)c(F)c1]

The titled compound (200 mg, 75%) was prepared in a manner similar to that in Example 207 step 4 from 3-(2,6-difluoro-4-((2-hydroxyethyl)(methyl)amino)phenyl)piperidine-2,6-dione and MsCl. [M+H]⁺=377.1.

### Step 3: 3-(4-((2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)(methyl)amino)-2,6-difluorophenyl)piperidine-2,6-dione

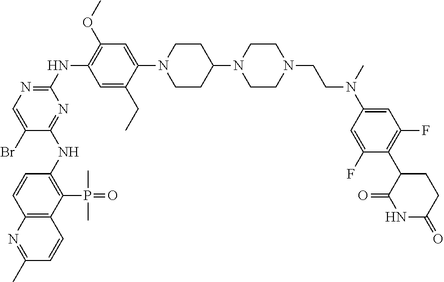 [SMILES: CCc1cc(Nc2ncc(Br)c(Nc3ccc4nc(C)ccc4c3P(C)(C)=O)n2)c(OC)cc1N1CCC(N2CCN(CCN(C)c3cc(F)c(C4CCC(=O)NC4=O)c(F)c3)CC2)CC1]

The titled compound (12 mg, 23%) was prepared in a manner similar to that in Example 207 step 5 from (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide and 2-((4-(2,6-dioxopiperidin-3-yl)-3,5-difluorophenyl)(methyl)amino)ethyl methanesulfonate. ¹H NMR (500 MHz, DMSO) δ 11.76 (s, 1H), 10.85 (s, 1H), 8.56 (d, J=8.9 Hz, 1H), 8.29 (s, 1H), 8.21 (s, 1H), 7.98 (s, 1H), 7.87 (d, J=9.2 Hz, 1H), 7.42 (d, J=8.9 Hz, 1H), 7.37 (s, 1H), 6.74 (s, 1H), 6.35 (d, J=12.8 Hz, 2H), 4.02 (dd, J=12.5, 5.0 Hz, 1H), 3.76 (d, J=5.1 Hz, 3H), 3.44 (t, J=6.6 Hz, 2H), 2.97-2.88 (m, 5H), 2.78 (s, 1H), 2.66 (d, J=14.7 Hz, 6H), 2.54 (s, 4H), 2.43 (s, 6H), 2.29 (d, J=7.0 Hz, 3H), 2.07 (s, 1H), 1.98-1.95 (m, 7H), 1.84 (d, J=9.4 Hz, 2H), 1.55 (d, J=9.9 Hz, 2H), 0.77 (s, 3H); [M+H]⁺=987.7.

### Example 434: 3-(5-(3-(4-(1-(4-((5-bromo-4-((5-(dimethylphosphoryl)-2-(trifluoromethyl)quinolin-6-yl)amino)pyrimidin-2-yl)amino)-2-ethyl-5-methoxyphenyl)piperidin-4-yl)piperazine-1-carbonyl)azetidin-1-yl)-1-oxoisoindolin-2-yl)piperidine-2,6-dione

### Step 1: 1,1-diphenyl-N-(2-(trifluoromethyl)luinolin-6-yl)methanimine

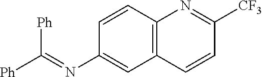 [SMILES: FC(F)(F)c1ccc2cc(N=C(c3ccccc3)c3ccccc3)ccc2n1]

To a mixture of 6-bromo-2-(trifluoromethyl)quinoline (2 g, 7.27 mmol), diphenylmethanimine (1.97 g, 10.9 mmol), Pd2(dba)₃ (668 mg, 0.73 mmol), BINAP (910 mg, 1.46 mmol), Cs2CO₃ (4.74 g, 14.5 mmol) in dioxane (60 mL) was stirred at 100° C. under N2 atmosphere for 18 hrs. After cooling to r.t, the reaction mixture was filtered, the filtrate was concentrated in vacuo. The residue was purified by silica gel column (PE: EA=4:1). 1,1-diphenyl-N-(2-(trifluoromethyl)quinolin-6-yl)methanimine (2.6 g, 95%) was obtained. [M+H]⁺=377.2.

### Step 2: 2-(trifluoromethyl)quinolin-6-amine

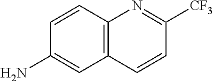 [SMILES: Nc1ccc2nc(C(F)(F)F)ccc2c1]

To a solution of 1,1-diphenyl-N-(2-(trifluoromethyl)quinolin-6-yl)methanimine (2.6 g, 6.9 mmol) in THF (30 mL) was added HCl (6 mL, 2N). The mixture was stirred at 20° C. for 30 mins and then sat. aq. Na2CO₃ solution was added to PH=8-9, the resulting mixture was extracted with EtOAc (50 mL×3). The combined organic phase was washed with brine (50 mL), dried over Na₂SO₄, filtered and concentrated in vacuum. The residue was purified by silica gel column (PE: EA=1:1). 2-(trifluoromethyl)quinolin-6-amine (1.2 g, 82%) was obtained. [M+H]⁺=213.2.

### Step 3: 5-iodo-2-(trifluoromethyl)quinolin-6-amine

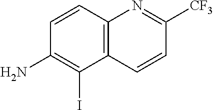 [SMILES: Nc1ccc2nc(C(F)(F)F)ccc2c1I]

To a solution of 2-(trifluoromethyl)quinolin-6-amine (1.2 g, 5.63 mmol) in HOAc (10 mL) was added ICl (1.06 g, 8.45 mmol). The mixture was stirred at 20° C. for 3 hrs and then sat. aq. Na2CO3 solution was added to PH=8-9, the resulting mixture was extracted with EtOAc (70 mL×3). The combined organic phase was washed with brine (60 mL), dried over Na₂SO₄, filtered and concentrated in vacuum. 5-iodo-2-(trifluoromethyl)quinolin-6-amine (1.7 g, 88%) was obtained. [M+H]⁺=339.1.

### Step 4: (6-amino-2-(trifluoromethyl)quinolin-5-yl)dimethylphosphine oxide

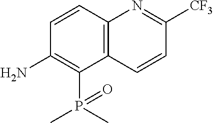 [SMILES: CP(C)(=O)c1c(N)ccc2nc(C(F)(F)F)ccc12]

To a mixture of 5-iodo-2-(trifluoromethyl)quinolin-6-amine (1.7 g, 5.01 mmol), dimethylphosphine oxide (496 mg, 6.52 mmol), Pd(OAc)2 (112 mg, 0.5 mmol), Xantphos (578 mg, 1 mmol), K3PO4 (2.12 g, 10.02 mmol) in dioxane (40 mL) was stirred at 100° C. under N2 atmosphere for 8 hrs. After cooling to r.t, the reaction mixture was filtered, the filtrate was concentrated in vacuo. The residue was purified by silica gel column (DCM:CH3OH=15:1). (6-amino-2-(trifluoromethyl)quinolin-5-yl)dimethylphosphine oxide (1.2 g, 83%) was obtained. [M+H]⁺=289.3.

### Step 5: (6-((5-bromo-2-chloropyrimidin-4-yl)amino)-2-(trifluoromethyl)quinolin-5-yl)dimethylphosphine oxide

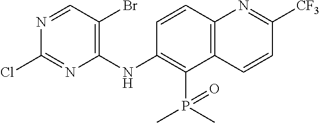 [SMILES: CP(C)(=O)c1c(Nc2nc(Cl)ncc2Br)ccc2nc(C(F)(F)F)ccc12]

To a solution of (6-amino-2-(trifluoromethyl)quinolin-5-yl)dimethylphosphine oxide (1 g, 3.46 mmol) in DMF (16 mL) was added NaH (346 mg, 8.65 mmol, 60%) at 0° C. The mixture was stirred at 0° C. for 20 min, then 5-bromo-2,4-dichloropyrimidine (1.56 g, 6.92 mmol) was added, the resulting solution was stirred at r.t for 2 hrs. The reaction was quenched by sat. aq. NH4Cl solution and the resulting mixture was extracted with EtOAc (30 mL×3). The combined organic phase was washed with brine (30 mL), dried over Na₂SO₄, filtered and concentrated in vacuum. The residue was purified by silica gel column (DCM:CH3OH=15:1). (6-((5-bromo-2-chloropyrimidin-4-yl)amino)-2-(trifluoromethyl)quinolin-5-yl)dimethylphosphine oxide (600 mg, 36.3%) was obtained. [M+H]⁺=479.2.

### Step 6: (6-((5-bromo-2-((5-ethyl-2-methoxy-4-(4-(piperazin-1-yl)piperidin-1-yl)phenyl)amino)pyrimidin-4-yl)amino)-2-(trifluoromethyl)quinolin-5-yl)dimethylphosphine oxide

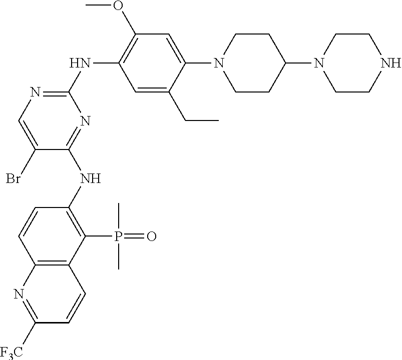 [SMILES: CCc1cc(Nc2ncc(Br)c(Nc3ccc4nc(C(F)(F)F)ccc4c3P(C)(C)=O)n2)c(OC)cc1N1CCC(N2CCNCC2)CC1]

To a mixture of (6-((5-bromo-2-chloropyrimidin-4-yl)amino)-2-(trifluoromethyl)quinolin-5-yl)dimethylphosphine oxide (400 mg, 0.84 mmol), tert-butyl 4-(1-(4-amino-2-ethyl-5-methoxyphenyl)piperidin-4-yl)piperazine-1-carboxylate (349 mg, 0.84 mmol), TsOH·H2O (479 mg, 2.52 mmol) in n-BuOH (8 mL) was stirred at 100° C. for 14 hrs. After cooling to r.t, the reaction mixture was concentrated in vacuo. The residue was diluted with DCM, washed with NaOH solution (20 mL, 1N) and brine, dried over Na2SO4 and then concentrated in vacuo. The residue was purified by silica gel column (DCM: CH3OH (0.05 NH3H2O)=2:1). (6-((5-bromo-2-((5-ethyl-2-methoxy-4-(4-(piperazin-1-yl)piperidin-1-yl)phenyl)amino)pyrimidin-4-yl)amino)-2-(trifluoromethyl)quinolin-5-yl)dimethylphosphine oxide (370 mg, 58%) was obtained. [M+H]⁺=761.3.

### Step 7: 3-(5-(3-(4-(1-(4-((5-bromo-4-((5-(dimethylphosphoryl)-2-(trifluoromethyl)quinolin-6-yl)amino)pyrimidin-2-yl)amino)-2-ethyl-5-methoxyphenyl)piperidin-4-yl)piperazine-1-carbonyl)azetidin-1-yl)-1-oxoisoindolin-2-yl)piperidine-2,6-dione

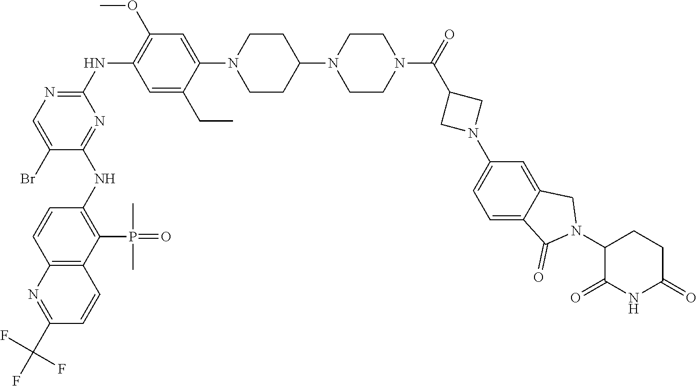 [SMILES: CCc1cc(Nc2ncc(Br)c(Nc3ccc4nc(C(F)(F)F)ccc4c3P(C)(C)=O)n2)c(OC)cc1N1CCC(N2CCN(C(=O)C3CN(c4ccc5c(c4)CN(C4CCC(=O)NC4=O)C5=O)C3)CC2)CC1]

The titled compound (6 mg, 16%) was prepared in a manner similar to that in Example 208 step 8 from (6-((5-bromo-2-((5-ethyl-2-methoxy-4-(4-(piperazin-1-yl)piperidin-1-yl)phenyl)amino)pyrimidin-4-yl)amino)-2-(trifluoromethyl)quinolin-5-yl)dimethylphosphine oxide and 1-(2-(2,6-dioxopiperidin-3-yl)-1-oxoisoindolin-5-yl)azetidine-3-carboxylic acid. ¹H NMR (500 MHz, DMSO) δ 12.16 (s, 1H), 10.94 (s, 1H), 8.89 (s, 1H), 8.49 (s, 1H), 8.34-8.10 (m, 2H), 8.03 (d, J=9.0 Hz, 1H), 7.89 (d, J=8.9 Hz, 1H), 7.50 (d, J=8.3 Hz, 1H), 7.29 (s, 1H), 6.78 (s, 1H), 6.57 (s, 1H), 6.52 (dd, J=8.3, 1.8 Hz, 1H), 5.04 (dd, J=13.3, 5.1 Hz, 1H), 4.31 (d, J=16.9 Hz, 1H), 4.19 (d, J=17.0 Hz, 1H), 4.13 (t, J=7.8 Hz, 2H), 4.00 (t, J=6.2 Hz, 2H), 3.92-3.87 (m, 1H), 3.50 (s, 2H), 3.35 (s, 3H), 2.95 (d, J=11.4 Hz, 2H), 2.91-2.83 (m, 1H), 2.68 (s, 2H), 2.57 (d, J=28.9 Hz, 4H), 2.42-2.25 (m, 5H), 2.05 (t, J=13.2 Hz, 6H), 1.99-1.89 (m, 2H), 1.82 (d, J=10.3 Hz, 2H), 1.65-1.45 (m, 2H), 0.79 (s, 3H). [M+H]⁺=1086.3.

### Example 435: 3-(4-(2-(4-(1-(4-((5-bromo-4-((5-(dimethylphosphoryl)-2-(trifluoromethyl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

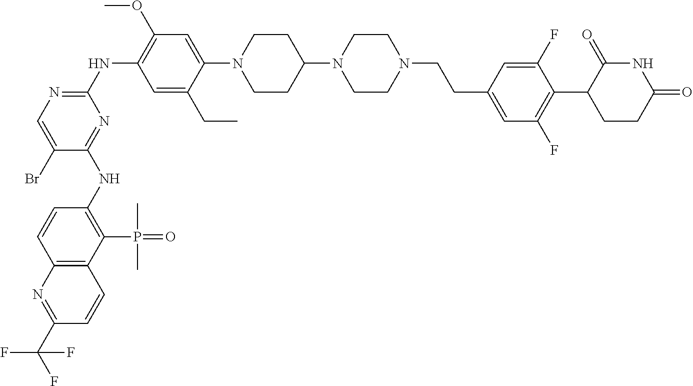 [SMILES: CCc1cc(Nc2ncc(Br)c(Nc3ccc4nc(C(F)(F)F)ccc4c3P(C)(C)=O)n2)c(OC)cc1N1CCC(N2CCN(CCc3cc(F)c(C4CCC(=O)NC4=O)c(F)c3)CC2)CC1]

The titled compound was prepared in a manner similar to that in Example 200. ¹H NMR (500 MHz, DMSO) δ 8.89 (d, J=9.3 Hz, 1H), 8.50 (s, 1H), 8.26 (s, 1H), 8.05 (d, J=9.7 Hz, 1H), 7.89 (d, J=9.1 Hz, 1H), 7.29 (s, 1H), 7.03 (d, J=10.1 Hz, 2H), 6.78 (s, 1H), 4.20 (dd, J=12.8, 4.7 Hz, 1H), 3.76 (s, 3H), 3.31 (d, J=11.8 Hz, 2H), 3.12 (s, 1H), 2.94 (d, J=9.6 Hz, 3H), 2.87-2.73 (m, 4H), 2.70-2.60 (m, 5H), 2.54 (s, 4H), 2.32-2.24 (m, 4H), 2.13 (d, J=9.4 Hz, 1H), 2.03 (t, J=13.6 Hz, 8H), 1.83 (s, 2H), 1.53 (s, 2H), 0.79 (s, 3H). [M+H]⁺=1012.3.

### Example 436: 3-(5-(4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)piperidin-1-yl)-3,3-dimethyl-2-oxoindolin-1-yl)piperidine-2,6-dione Step 1: tert-butyl 1-(1-(2,6-bis(benzyloxy)pyridin-3-yl)-3,3-dimethyl-2-oxoindolin-5-yl)piperidine-4-carboxylate

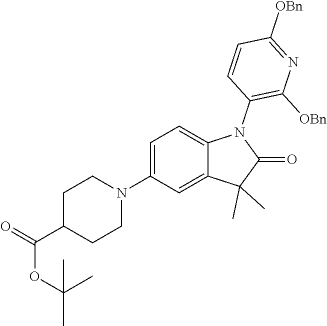 [SMILES: CC(C)(C)OC(=O)C1CCN(c2ccc3c(c2)C(C)(C)C(=O)N3c2ccc(OCc3ccccc3)nc2OCc2ccccc2)CC1]

A mixture of 1-(2,6-bis(benzyloxy)pyridin-3-yl)-5-bromo-3,3-dimethylindol-2-one (the material was obtained through the similar method of example 409) (1.0 g, 1.889 mmol) and tert-butyl piperidine-4-carboxylate (419.9 mg, 2.267 mmol), Cs₂CO₃ (923.1 g, 2.833 mmol), XPhos (180.1 mg, 0.378 mmol), Pd₂(dba)₃ (173.0 mg, 0.189 mmol) in dioxane (20 mL) was stirred overnight at 110° C. under nitrogen atmosphere. The resulting mixture was filtered, the filter cake was washed with EtOAc (150 mL). The filtrate was concentrated under reduced pressure. The residue was purified by Prep-TLC (PE/EtOAc 1:1) to afford product (620.0 mg, 51.7%). [M+H]⁺=634.1.

### Step 2: tert-butyl 1-(1-(2,6-dioxopiperidin-3-yl)-3,3-dimethyl-2-oxoindolin-5-yl)piperidine-4-carboxylate

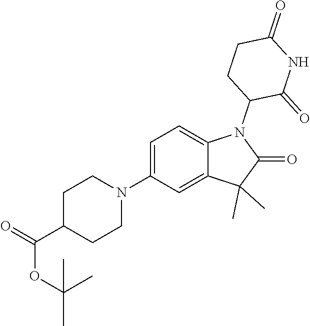 [SMILES: CC(C)(C)OC(=O)C1CCN(c2ccc3c(c2)C(C)(C)C(=O)N3C2CCC(=O)NC2=O)CC1]

To a stirred mixture of tert-butyl 1-(1-(2,6-bis(benzyloxy)pyridin-3-yl)-3,3-dimethyl-2-oxoindolin-5-yl)piperidine-4-carboxylate (620.0 mg, 0.978 mmol) and Pd/C (300.0 mg, 10% wt) in EtOH (10 mL) was added AcOH (50 mL, 872 mmol) dropwise and stirred overnight at 50° C. under hydrogen atmosphere. The resulting mixture was filtered, the filter cake was washed with MeOH (150 mL). The filtrate was concentrated under reduced pressure to afford the product (300.0 mg, 67.3%). [M+H]⁺=456.2.

### Step 3: 1-(1-(2,6-dioxopiperidin-3-yl)-3 ,3-dimethyl-2-oxoindolin-5-yl)piperidine-4-carboxylic acid

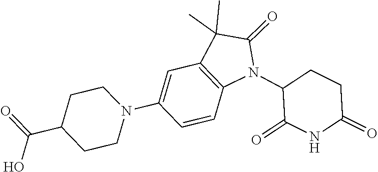 [SMILES: CC1(C)C(=O)N(C2CCC(=O)NC2=O)c2ccc(N3CCC(C(=O)O)CC3)cc21]

A mixture of tert-butyl 1-(1-(2,6-dioxopiperidin-3-yl)-3,3-dimethyl-2-oxoindolin-5-yl)piperidine-4-carboxylate (300.0 mg, 0.659 mmol) in TFA (1.5 mL) and DCM (3 mL) was stirred for 4 h at room temperature. The resulting mixture was concentrated under reduced pressure. The residue was purified by trituration with EtOAc (10 mL). 1-(1-(2,6-dioxopiperidin-3-yl)-3,3-dimethyl-2-oxoindolin-5-yl)piperidine-4-carboxylic acid (158.8 mg, 60.3%) was obtained. [M+H]⁺=400.2.

### Step 4: 3-(5-(4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)piperidin-1-yl)-3,3-dimethyl-2-oxoindolin-1-yl)piperidine-2,6-dione

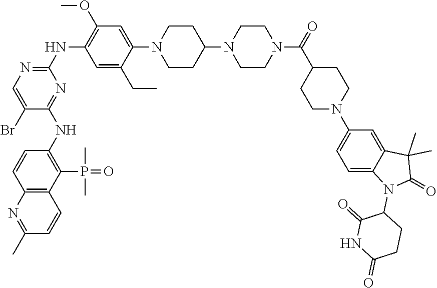 [SMILES: CCc1cc(Nc2ncc(Br)c(Nc3ccc4nc(C)ccc4c3P(C)(C)=O)n2)c(OC)cc1N1CCC(N2CCN(C(=O)C3CCN(c4ccc5c(c4)C(C)(C)C(=O)N5C4CCC(=O)NC4=O)CC3)CC2)CC1]

The titled compound (12 mg, 17%) was prepared in a manner similar to that in Example 208 step 8 from (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide and 1-(1-(2,6-dioxopiperidin-3-yl)-3,3-dimethyl-2-oxoindolin-5-yl)piperidine-4-carboxylic acid. ¹H NMR (500 MHz, DMSO) δ 11.76 (s, 1H), 11.04 (s, 1H), 8.56 (d, J=8.8 Hz, 1H), 8.30 (s, 1H), 8.23 (d, J=16.5 Hz, 1H), 7.98 (s, 1H), 7.87 (d, J=9.2 Hz, 1H), 7.48-7.28 (m, 2H), 7.10 (d, J=1.9 Hz, 1H), 6.77 (dd, J=21.1, 10.8 Hz, 3H), 5.15 (s, 1H), 3.76 (s, 3H), 3.64-3.51 (m, 4H), 3.47 (s, 2H), 2.95 (d, J=10.6 Hz, 2H), 2.87 (t, J=13.2 Hz, 1H), 2.72 (d, J=7.6 Hz, 1H), 2.71-2.66 (m, 3H), 2.65 (s, 4H), 2.62-2.53 (m, 4H), 2.49-2.45 (m, 2H), 2.37 (t, J=11.1 Hz, 1H), 2.30 (d, J=6.9 Hz, 2H), 1.98 (d, J=13.3 Hz, 6H), 1.93 (dd, J=10.5, 5.2 Hz, 1H), 1.83 (d, J=10.2 Hz, 2H), 1.77-1.65 (m, 4H), 1.56 (dt, J=30.8, 15.3 Hz, 2H), 1.26 (t, J=11.3 Hz, 6H), 0.77 (s, 3H). [M+H]⁺=1088.4.

### Example 437: 3-(4-(4-(2-(4-(1-(4-((5-bromo-4-((5-(dimethylphosphoryl)-2-(trifluoromethyl)quinolin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperdine-2,6-dione

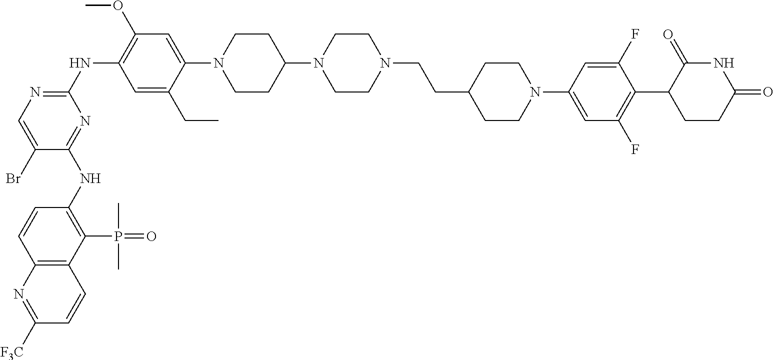 [SMILES: CCc1cc(Nc2ncc(Br)c(Nc3ccc4nc(C(F)(F)F)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1]

The titled compound was prepared in a manner similar to that in Example 204 Step 6 from 2-(1-(4-(2,6-dioxopiperidin-3-yl)-3,5-difluorophenyl)piperidin-4-yl)acetaldehyde and (6-((5-bromo-2-((5-ethyl-2-methoxy-4-(4-(piperazin-1-yl)piperidin-1-yl)phenyl)amino)pyrimidin-4-yl)amino)-2-(trifluoromethyl)quinolin-5-yl)dimethylphosphine oxide. ¹H NMR (500 MHz, DMSO) δ 12.16 (s, 1H), 10.86 (s, 1H), 8.89 (s, 1H), 8.48 (s, 1H), 8.25 (s, 1H), 8.17 (d, J=13.9 Hz, 1H), 8.03 (d, J=9.2 Hz, 1H), 7.88 (d, J=9.0 Hz, 1H), 7.28 (s, 1H), 6.78 (s, 1H), 6.60 (d, J=12.8 Hz, 2H), 4.04 (dd, J=12.5, 4.9 Hz, 1H), 3.74 (d, J=18.3 Hz, 5H), 2.93 (d, J=10.5 Hz, 2H), 2.84-2.72 (m, 2H), 2.68 (dd, J=19.8, 9.0 Hz, 4H), 2.53 (d, J=11.1 Hz, 3H), 2.48-2.45 (m, 1H), 2.39 (s, 3H), 2.35-2.21 (m, 6H), 2.10 (dd, J=12.9, 9.1 Hz, 1H), 2.04 (d, J=13.3 Hz, 6H), 1.98-1.90 (m, 1H), 1.83 (d, J=10.9 Hz, 2H), 1.76-1.64 (m, 2H), 1.53 (dd, J=20.2, 10.9 Hz, 3H), 1.42-1.31 (m, 2H), 1.26-1.08 (m, 2H), 0.79 (s, 3H). [M+H]⁺=1095.4.

### Example 484: (R)-3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: ethyl 4-(4-bromo-6-difluorophenyl)-4-cyanobutanoate

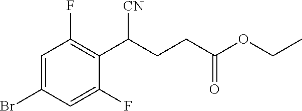 [SMILES: CCOC(=O)CCC(C#N)c1c(F)cc(Br)cc1F]

To a solution of 2-(4-bromo-2,6-difluorophenyl)acetonitrile (10 g, 43.1 mmol) in THF (150 mL) was added LDA (2 M in THF, 24 mL, 48 mmol) dropwise in 20 min at −65° C., the reaction solution was stirred for 1 hour at this temperature, then to this was added ethyl 3-bromopropanoate (9.4 g, 51.7 mmol) in THF (30 mL) dropwise in 10 min. The resulting solution was stirred for 30 min at −65° C., and then allowed to warm to room temperature naturally. The reaction was quenched by the addition of sat. aq. NH₄Cl (50 mL), and extracted with EtOAc (100 mL×3). The combined organic layers were washed with brine, dried over anhydrous Na₂SO₄. After filtration, the filtrate was concentrated under reduced pressure to afford the product (13.8 g, 96.5%). [M+H]⁺=332.0.

### Step 2: 4-(4-bromo-2,6-difluorophenyl)-4-cyanobutanoic acid

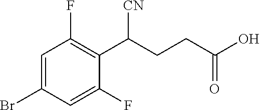 [SMILES: N#CC(CCC(=O)O)c1c(F)cc(Br)cc1F]

To a solution of ethyl 4-(4-bromo-2,6-difluorophenyl)-4-cyanobutanoate (13.5 g, 40.7 mmol) in THF/H₂O (90 mL/30 mL) was added LiOH (2.9 g, 0.122 mol). The reaction mixture was stirred for 12 h at room temperature. The resulting mixture was diluted with water, and extracted with EtOAc (50 mL×2). The pH value of water phase was adjusted to 4-5 with 1 N HCl (10 mL), and extracted with EtOAc (50 mL×3). The combined organic layers were washed with brine (50 mL×3), and dried over anhydrous Na₂SO₄. After filtration, the filtrate was concentrated under reduced pressure to afford the product (10.2 g, 82.5%). [M+H]⁺=304.2.

### Step 3: 3-(4-bromo-2,6-difluorophenyl)piperidine-2,6-dione

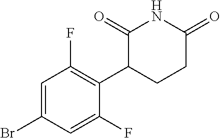 [SMILES: O=C1CCC(c2c(F)cc(Br)cc2F)C(=O)N1]

To a stirred solution of 4-(4-bromo-2,6-difluorophenyl)-4-cyanobutanoic acid (10.2 g, 33.5 mmol) in toluene (100 mL) was added conc. H₂SO₄ (2 mL, 36.9 mmol). The resulting solution was stirred at 100° C. for 3 h. The reaction mixture was concentrated under vacuum, then the mixture was poured into water. The pH value was adjusted to 7-8 with sat. aq. NaHCO₃(40 mL), and resulting solution was extracted with EtOAc (50 mL×3). The combined organic layers were washed with water and brine, dried over anhydrous Na₂SO₄, filtered and concentrated to afford the product (8.2 g, 80.4%). [M+H]⁺=304.3.

### Step 4: (R,E)-3-(4-(2-ethoxyvinyl)-2,6-difluorophenyl)piperidine-2,6-dione

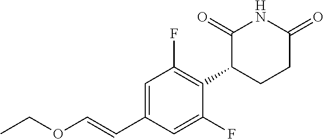 [SMILES: CCO/C=C/c1cc(F)c([C@H]2CCC(=O)NC2=O)c(F)c1]

To a stirred solution of 3-(4-bromo-2,6-difluorophenyl)piperidine-2,6-dione (8.2 g, 27.0 mmol) and (E)-2-(2-ethoxyvinyl)-4,4,5,5-tetramethyl-1,3,2-dioxaborolane (6.4 g, 32.4 mmol) in DMF/H₂O (100 mL/20 mL) were added Pd(dtbpf)Cl₂ (883 mg, 1.35 mmol) and CsF (8.2 g, 54.0 mmol). The resulting mixture was stirred for 2 h at 80° C. under nitrogen atmosphere. The reaction solution was diluted with water, extracted with EtOAc (100 mL×2). The combined organic layers were washed with water and brine, dried over anhydrous Na₂SO₄, filtered and concentrated under reduced pressure. The residue was purified by SFC (IH (3*25 cm, 5 um), 13% EtOH/87% CO2, 100 bar, 100 ml/min) and the title compound corresponded to peak A @ 1.679 min/254 nm. (3.1 g, 39.0%). [M+H]⁺=296.1.

### Step 5: (R)-2-(4-(2,6-dioxopiperidin-3-yl)-3,5-difluorophenyl)acetaldehyde

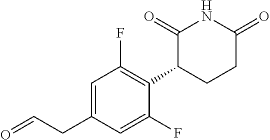 [SMILES: O=CCc1cc(F)c([C@H]2CCC(=O)NC2=O)c(F)c1]

(R,E)-3-(4-(2-ethoxyvinyl)-2,6-difluorophenyl)piperidine-2,6-dione (3.1 g, 10.4 mmol) was dissolved in FA(50 mL). The resulting solution was stirred for 2 h at room temperature. The reaction solution was evaporated to dryness to afford the product (2.6 g, 91.8%). [M+H]⁺=268.1.

### Step 6: 6-nitroquinolin-2-ol

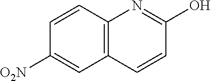 [SMILES: O=[N+]([O-])c1ccc2nc(O)ccc2c1]

To a stirred solution of quinolin-2-ol (6 g, 41.3 mmol) in conc. H₂SO₄ (98%, 50 mL) was added dropwise a solution of conc. HNO₃ (65%, 3.12 g, 49.6 mmol) at 0° C. Then the mixture was stirred at rt for 1h. The mixture was diluted with water (200 mL) at 0° C. The resulting mixture was filtered and the filter cake was washed with H₂O (500 ml), and dried in vacuum to afford 6-nitroquinolin-2-ol (5.5 g 69.9%) [M+H]⁺=191.1.

### Step 7: 2-chloro-6-nitroquinoline

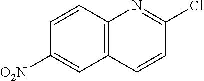 [SMILES: O=[N+]([O-])c1ccc2nc(Cl)ccc2c1]

A solution of 6-nitroquinolin-2-ol (5.5 g, 28.78 mmol) in POCl₃ (50 mL) was stirred at 100° C. for 2 hrs. Then the mixture was cooled to rt, and concentrated in vacuo. The residue was purified by Combi-Flash (silica column, 40 g, DCM:MeOH=15:1) to give 2-chloro-6-nitroquinoline(5 g, 82.9%) [M+H]⁺=209.1.

### Step 8: 6-nitro-2-vinylquinoline

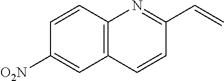

To a suspension of 2-chloro-6-nitroquinoline (5 g, 23.9 mmol) and 4,4,5,5-tetramethyl-2-vinyl-1,3,2-dioxaborolane (7.37 g, 47.8 mmol) in dioxane (40 mL) and water (10 mL) was added K₂CO3 (9.91 g, 71.8 mmol) and Pd(dppf)Cl₂ (1.74 g, 2.39 mmol) under nitrogen atmosphere. The mixture was warmed to 100° C. and stirred for 16 hrs. Then the mixture was cooled to rt and filtered. The filtrate was concentrated in vacuo. The residue was purified by Combi-Flash (silica column, 40 g, DCM:MeOH=15:1) to give 6-nitro-2-vinylquinoline (4.5 g, 93.9%). [M+H]⁺=201.1.

### Step 9: 2-ethylquinolin-6-amine

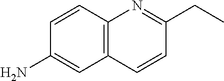

To a suspension of 6-nitro-2-vinylquinoline (4.5 g, 22.38 mmol) in MeOH (20 mL) was added Pd/C (10 wt. %, wet, 1.5 g). The mixture was stirred at rt for 16 hrs under hydrogen atmosphere. Then the mixture was filtered and the solid was washed with MeOH. The filtrate was concentrated in vacuo to afford 2-ethylquinolin-6-amine (3.84 g, 99.2%). [M+H]⁺=173.1.

### Step 11: 2-ethyl-5-iodoquinolin-6-amine

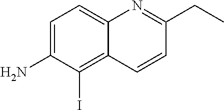

The title compound (4.5 g, 75.3%) was prepared in a manner similar to that in Example 486 step 4 from 2-ethylquinolin-6-amine and ICl [M+H]⁺=299.1.

### Step 12: (6-amino-2-ethylquinolin-5-yl)dimethylphosphine oxide

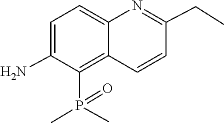 [SMILES: CCc1ccc2c(P(C)(C)=O)c(N)ccc2n1]

The title compound (3.5 g, 93.5%). was prepared in a manner similar to that in Example 486 step 5 from 2-ethyl-5-iodoquinolin-6-amine and dimethylphosphineoxide. [M+H]⁺=249.1.

### Step 13: (6-((5-bromo-2-chloropyrimidin-4-yl)amino)-2-ethylquinolin-5-yl)dimethylphosphine oxide

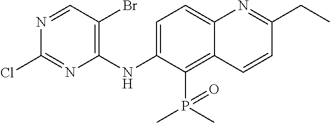 [SMILES: CCc1ccc2c(P(C)(C)=O)c(Nc3nc(Cl)ncc3Br)ccc2n1]

The title compound (2.5 g, 40.4%). was prepared in a manner similar to that in Example 486 step 6 from (6-amino-2-ethylquinolin-5-yl)dimethylphosphine oxide and 5-bromo-2,4-dichloropyrimidine. [M+H]⁺=439.6.

### Step 14: (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide

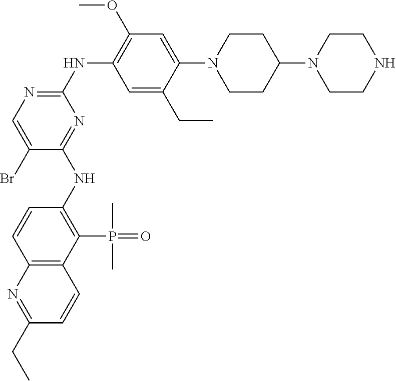 [SMILES: CCc1ccc2c(P(C)(C)=O)c(Nc3nc(Nc4cc(CC)c(N5CCC(N6CCNCC6)CC5)cc4OC)ncc3Br)ccc2n1]

The title compound (2.0 g, 48.8%). was prepared in a manner similar to that in Example 486 step 7 from (6-((5-bromo-2-chloropyrimidin-4-yl)amino)-2-ethylquinolin-5-yl)dimethylphosphine oxide and tert-butyl 4-(1-(4-amino-5-ethoxy-2-ethylphenyl)piperidin-4-yl)piperazine-1-carboxylate. [M+H]⁺=721.5.

### Step 15: (R)-3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

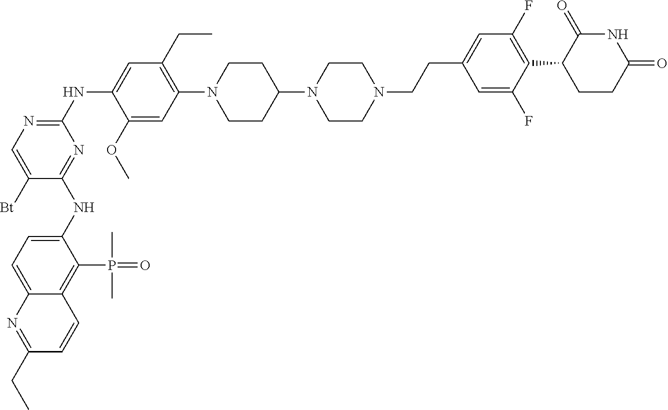

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (500 mg, 0.694 mmol) and (R)-2-(4-(2,6-dioxopiperidin-3-yl)-3,5-difluorophenyl)acetaldehyde (222.49 mg, 0.832 mmol) in DCM (8 mL) was stirred in a flask at room temperature for 2 hour. To the mixture was added sodium triacetoxyborohydride (146.34 mg, 0.694 mmol) and the reaction was stirred at room temperature for another 2 h. The resulting mixture was diluted with H₂O (60 mL) and the layers were separated. The aqueous layer was extracted with DCM (3×30 mL). The combined organic layers were washed with brine (50 mL×3), dried over anhydrous Na₂SO₄, filtered and concentrated under reduced pressure. The residue was purified by silica gel column chromatography to afford the crude product (600 mg), which was purified with prep-HPLC chromatography (0.1% FA in water: acetonitrile=90:10˜ 50:50 gradient elution) to give the product (480 mg, 71.2%). ¹H NMR (500 MHz, DMSO) δ 11.73 (s, 1H), 10.88 (s, 1H), 8.49 (d, J=8.8 Hz, 1H), 8.20 (s, 1H), 8.15 (d, J=12.4 Hz, 1H), 7.94 (s, 1H), 7.80 (d, J=9.4 Hz, 1H), 7.37 (d, J=8.9 Hz, 1H), 7.26 (s, 1H), 6.96 (d, J=10.0 Hz, 2H), 6.68 (s, 1H), 4.13 (dd, J=12.6, 5.0 Hz, 1H), 3.69 (s, 3H), 2.86 (dd, J=15.2, 7.6 Hz, 4H), 2.79-2.65 (m, 4H), 2.59 (t, J=11.3 Hz, 3H), 2.47 (s, 4H), 2.41-2.31 (m, 4H), 2.22 (d, J=4.7 Hz, 3H), 2.05 (s, 2H), 1.92 (d, J=13.3 Hz, 7H), 1.77 (d, J=10.2 Hz, 2H), 1.47 (d, J=8.8 Hz, 2H), 1.25 (t, J=7.6 Hz, 3H), 0.70 (s, 3H). [M+H]⁺=972.7

### Example 486: (R)-3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

### Step 1: 6-bromo-2-ethyl-8-fluoroquinoline

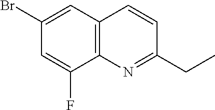

To a stirred mixture of 4-bromo-2-fluoroaniline (10 g, 52.6 mmol) and BTEAC (1 g) in aqueous HCl (6M) was added (E)-pent-2-enal (8.84 g, 105.3 mmol) in toluene (50 mL) dropwise at 100° C. The resulting mixture was stirred for 15 h at 100° C. under nitrogen atmosphere. The reaction was extracted with EA. The aqueous layer was concentrated under reduced pressure. The residue was dissolved in water, and the pH was adjusted to 8-9 with aqueous NaOH (3M). The resulting mixture was extracted with DCM (100 mL×3). The combined organic layers were washed with brine (100 mL×3), dried over anhydrous Na₂SO₄, filtered and concentrated under reduced pressure. The crude product was purified by column chromatography (0-15% EA in PE) to afford product (3.5 g, 26.2%). [M+H]⁺=254.1.

### Step 2: tert-butyl (2-ethyl-8-fluoroquinolin-6-yl)carbamate

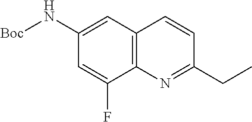 [SMILES: CCc1ccc2cc(NC(=O)OC(C)(C)C)cc(F)c2n1]

A mixture of 6-bromo-2-ethyl-8-fluoroquinoline (3.5 g, 13.8 mmol), BocNH₂ (1.93 g, 16.5 mmol), Pd₂(dba)₃ (631 mg, 0.69 mmol), XantPhos (799 mg, 1.38 mmol), and Cs₂CO₃ (11.2 g, 34.5 mmol) in dioxane (80 mL) was stirred at 100° C. under nitrogen atmosphere for 15 hrs. After cooling to r.t, the reaction mixture was filtered and concentrated in vacuo. The residue was purified by silica gel column (PE: EA=4:1) to afford product (2.8 g, 70%). [M+H]⁺=291.1.

### Step 3: 2-ethyl-8-fluoroquinolin-6-amine

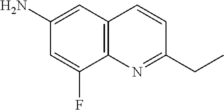

To a stirred solution of tert-butyl (2-ethyl-8-fluoroquinolin-6-yl)carbamate (2.8 g, 9.7 mmol) in DCM (20 mL) was added TFA (10 mL). The reaction mixture was stirred at rt for 2 h and concentrated in vacuum to afford the product (1.8 g, 98.5%) [M+H]⁺=191.1.

### Step 4: 2-ethyl-8-fluoro-5-iodoquinolin-6-amine

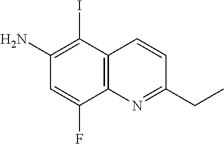

To a solution of 2-ethyl-8-fluoroquinolin-6-amine (1.8 g, 9.5 mmol) in HOAc (20 mL) was added ICl (1.84 g, 11.4 mmol). The mixture was stirred at 20° C. for 3 hrs and then sat. aq. Na₂CO₃ was added to adjust the PH to 8-9. The resulting mixture was extracted with EtOAc (50 mL×3). The combined organic layers were washed with brine (60 mL×3), dried over Na₂SO₄, filtered and concentrated under reduced pressure. 2-Ethyl-8-fluoro-5-iodoquinolin-6-amine (1.7 g, 56.7%) was obtained. [M+H]⁺=317.0.

### Step 5: (6-amino-2-ethyl-8-fluoroquinolin-5-yl)dimethylphosphine oxide

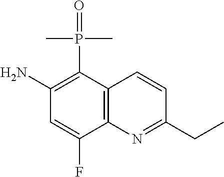 [SMILES: CCc1ccc2c(P(C)(C)=O)c(N)cc(F)c2n1]

A mixture of 2-ethyl-8-fluoro-5-iodoquinolin-6-amine (1.7 g, 5.4 mmol), dimethylphosphineoxide (627 mg, 8 mmol), K₃PO₄ (2.8 g, 13.5 mmol), Pd(OAc)2 (120 mg, 0.54 mmol) and XantPhos (310 mg, 0.54 mmol) in dioxane (30 mL) was stirred for 3 h at 100° C. under nitrogen atmosphere. After cooling to room temperature, the mixture was concentrated under vacuum, and the residue was purified by column chromatography (0-10% MeOH in DCM) to afford the product (1.2 g, 83.3%). [M+H]⁺=267.1.

### Step 6: (6-((5-bromo-2-chloropyrimidin-4-yl)amino)-2-ethyl-8-fluoroquinolin-5-yl)dimethylphosphine oxide

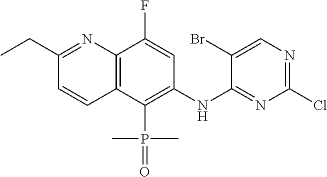 [SMILES: CCc1ccc2c(P(C)(C)=O)c(Nc3nc(Cl)ncc3Br)cc(F)c2n1]

To a solution of (6-amino-2-ethyl-8-fluoroquinolin-5-yl)dimethylphosphine oxide (1.2 g, 4.5 mmol) in THF (30 mL) was added 5-bromo-2,4-dichloropyrimidine (2.6 g, 11.2 mmol). LiHMDS (1 M in THF, 9 mL, 9 mmol) was added to the reaction mixture at 0° C. The mixture was stirred at 20° C. for 3 hrs. The mixture was diluted with water (20 mL), and the layers were separated. The aqueous layer was extracted with DCM (20 mL×3). The combined organic layers were washed with brine (20 mL×3), dried over Na₂SO₄, filtered and concentrated under reduced pressure. The residue was purified by column chromatography (DCM/MeOH=20/1 to 10/1) to afford prodcut (1.3 g, 63.4%). [M+H]⁺=457.0.

### Step 7: (6-((5-bromo-2-((2-ethoxy-5-ethyl-4-(4-(piperazin-1-yl)piperidin-1-yl)phenyl)amino)pyrimidin-4-yl)amino)-2-ethyl-8-fluoroquinolin-5-yl)dimethylphosphine oxide

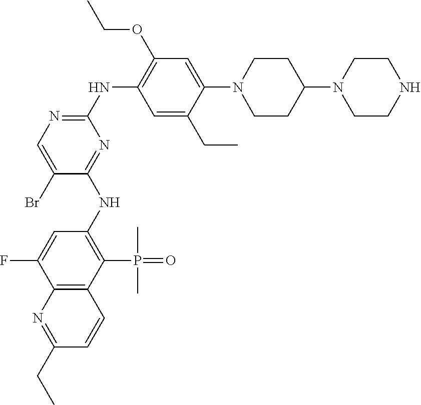 [SMILES: CCOc1cc(N2CCC(N3CCNCC3)CC2)c(CC)cc1Nc1ncc(Br)c(Nc2cc(F)c3nc(CC)ccc3c2P(C)(C)=O)n1]

To a stirred solution of (6-((5-bromo-2-chloropyrimidin-4-yl)amino)-2-ethyl-8-fluoroquinolin-5-yl)dimethylphosphine oxide (800 mg, 1.7 mmol) and tert-butyl 4-(1-(4-amino-5-ethoxy-2-ethylphenyl)piperidin-4-yl)piperazine-1-carboxylate (755 mg, 1.7 mmol) in n-BuOH (40 mL) was added TsOH (877 mg, 5.1 mmol). The resulting mixture was stirred at 95° C. for 16 hours. The reaction mixture was concentrated under vacuum before aqueous NaOH (1M, 10 mL) was added into the mixture. Then the mixture was extracted with DCM (3×20 mL). The combined organic layers were washed with brine (3×20 mL), dried over Na₂SO₄, filtered and concentrated under reduced pressure to afford the crude residue, which was purified with silica gel column chromatography (DCM:MeOH=6:1) to give the title product (790 mg, 60.8%). [M+H]⁺=753.3.

### Step 8: (R)-3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

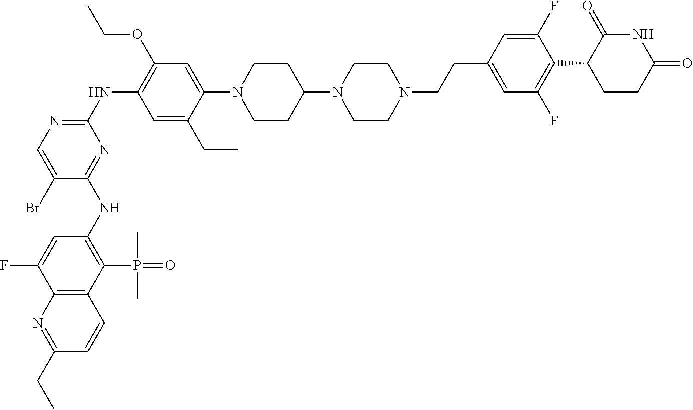 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2cc(F)c3nc(CC)ccc3c2P(C)(C)=O)n1]

To a solution of (6-((5-bromo-2-((2-ethoxy-5-ethyl-4-(4-(piperazin-1-yl)piperidin-1-yl)phenyl)amino)pyrimidin-4-yl)amino)-2-ethyl-8-fluoroquinolin-5-yl)dimethylphosphine oxide (50 mg, 0.07 mmol) and (R)-2-(4-(2,6-dioxopiperidin-3-yl)-3,5-difluorophenyl)acetaldehyde (40 mg, 0.15 mmol) in DCM (3 mL) was added STAB (32 mg, 0.15 mmol) at 20° C. The mixture was stirred at 20° C. for 1 hr. The mixture was diluted with water (20 mL), and the layers were separated. The aqueous layer was extracted with DCM (20 mL×3). The combined organic layers were washed with brine (20 mL×3), dried over Na₂SO₄, filtered and concentrated under reduced pressure. The residue was purified by prep-HPLC (C-18 column chromatography (0.1% FA in water: acetonitrile=90: 10˜ 60: 40 gradient elution) to afford the product (24.5 mg, 19.6%). ¹H NMR (500 MHz, DMSO) δ 12.04 (s, 1H), 10.88 (s, 1H), 8.50 (d, J=8.5 Hz, 1H), 8.31-8.11 (m, 2H), 7.97 (s, 1H), 7.47 (d, J=9.0 Hz, 1H), 7.33 (s, 1H), 6.95 (d, J=10.1 Hz, 2H), 6.64 (s, 1H), 4.13 (dd, J=12.6, 4.9 Hz, 1H), 3.94 (q, J=6.9 Hz, 2H), 2.86-2.91 (m, 5H), 2.67-2.78 (m, 3H), 2.45-2.60 (m, 9H), 2.15-2.42 (m, 7H), 2.00-2.10 (m, 1H), 1.93 (d, J=13.3 Hz, 6H), 1.74-1.77 (m, 2H), 1.36-1.46 (m, 2H), 1.25 (t, J=7.6 Hz, 3H), 1.19 (t, J=6.9 Hz, 3H), 0.68 (s, 3H). [M+H]⁺=1004.7.

### Example 488: (R)-3-(4-((R)-3-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

### Step 1: 3-fluoroquinolin-2(1H)-one

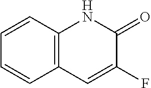 [SMILES: O=c1[nH]c2ccccc2cc1F]

To a solution of quinolin-2(1H)-one (25.0 g, 0.17 mol) in MeCN (30 mL) was added Selectfluor (64.0 g, 0.18 mol) at room temperature. The resulting mixture was stirred at 80° C. overnight. The reaction was concentrated to give the crude residue, which was purified by silica gel column chromatography (DCM: MeOH=100: 0˜20: 1 gradient elution) to give a mixture (10.4 g, 37%) containing the desired product. [M+H]⁺=164.2.

### Step 2: 3-fluoro-6-nitroquinolin-2(1H)-one

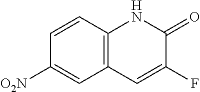 [SMILES: O=c1[nH]c2ccc([N+](=O)[O-])cc2cc1F]

To the mixture from above step (10.4 g, 63.4 mmol) in conc. H₂SO₄ (98%, 80 mL) was added conc. HNO₃ (65%, 7.4 g, 76.1 mmol) at 0° C. The resulting mixture was stirred at 0° C. for 1 hour. The reaction was poured into ice water (200 mL) and stirred for 10 mins. The mixture was filtered and the solid was washed with water (50 mL×2), dried under reduced pressure to give the crude product (7.5 g, 56.8%).

¹H NMR (500 MHz, DMSO) δ 12.84 (s, 1H), 8.68 (d, J=2.5 Hz, 1H), 8.31 (dd, J=9.1, 2.5 Hz, 1H), 8.09 (d, J=10.7 Hz, 1H), 7.47 (d, J=9.1 Hz, 1H). [M+H]⁺=209.2.

### Step 3: 2-chloro-3-fluoro-6-nitroquinoline

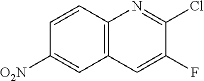 [SMILES: O=[N+]([O-])c1ccc2nc(Cl)c(F)cc2c1]

A solution of 3-fluoro-6-nitroquinolin-2(1H)-one (7.5 g, 35.9 mmol) in POCl₃ (60 mL) was heated at 100° C. overnight. The reaction was concentrated, basified with sat. aq. NaHCO₃, and then extracted with DCM (3×100 mL). The combined organic layers were dried over anhydrous Na₂SO₄, filtered and evaporated under reduced pressure to afford the crude residue, which was purified by silica gel column chromatography (PE: EA=100: 0˜10: 1 gradient elution) to give the desired product (3.7 g, 32%). [M+H]⁺=227.0.

### Step 4: 3-fluoro-2-methyl-6-nitroquinoline

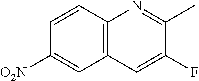

A mixture of 2-chloro-3-fluoro-6-nitroquinoline (3.7 g, 16.3 mmol), 2,4,6-trimethyl-1,3,5,2,4,6-trioxatriborinane (14.0 mL, 48.9 mmol), Pd(dppf)Cl₂ (1.2 g, 1.63 mmol) and K₃PO₄ (6.9 g, 32.6 mmol) in DME (100 mL) and H₂O (20 mL) was stirred in a round bottom flask at 80° C. overnight under nitrogen atmosphere. The mixture was evaporated under reduced pressure to afford the crude product, which was purified with silica gel column chromatography (PE: EA=100: 0˜20: 1 gradient elution) to give the title product (2.6 g, 77%). [M+H]⁺=207.1.

### Step 5: 3-fluoro-2-methylquinolin-6-amine

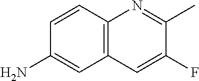

To a solution of 3-fluoro-2-methyl-6-nitroquinoline (2.6 g, 12.5 mmol) in MeOH (50 mL)/DCM (25 mL) was added Pd/C (10 wt. %, wet, 800 mg) at 25° C. under nitrogen atmosphere. The flask was evacuated and backfilled with hydrogen three times. After stirring under hydrogen atmosphere at 25° C. for 5 hours, the mixture was filtered through a pad of Celite and the solid was washed with MeOH (50 mL). The filtrate was concentrated under reduced pressure to afford the desired product (2.2 g, 99%). [M+H]⁺=177.1.

### Step 6: 3-fluoro-5-iodo-2-methylquinolin-6-amine

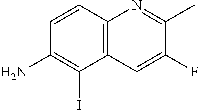

To a solution of 3-fluoro-2-methylquinolin-6-amine (2.2 g, 12.4 mmol) in AcOH (60 mL) was added ICl (16.1 mL, 16.1 mmol) at 20° C. Then the mixture was stirred at 20° C. for 1 hour. Then the mixture was adjusted to pH=8 with sat. aq. NaHCO₃. The resulting mixture was extracted with DCM (3×100 mL). The combined organic layers were washed with brine (3×80 mL), dried over Na₂SO₄, filtered and concentrated under reduced pressure to afford the desired product (3.3 g, 88%). [M+H]⁺=303.0.

### Step 7: (6-amino-3-fluoro-2-methylquinolin-5-yl)dimethylphosphine oxide

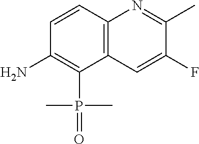 [SMILES: Cc1nc2ccc(N)c(P(C)(C)=O)c2cc1F]

To a solution of 3-fluoro-5-iodo-2-methylquinolin-6-amine (3.3 g, 10.9 mmol) and dimethylphosphine oxide (1.3 g, 16.3 mmol) in dioxane (120 mL) was added K₃PO₄ (6.9 g, 32.7 mmol) at 20° C. Pd(OAc)2 (244 mg, 1.09 mmol) and Xantphos (1.2 g, 2.18 mmol) were then added to the mixture at the same temperature. The flask was evacuated and backfilled with nitrogen three times, before the mixture was stirred at 100° C. overnight. The mixture was filtered and concentrated under reduced pressure. The residue was purified by silica gel column chromatography (DCM: MeOH=100: 0˜10: 1 gradient elution) to give the desired product (2.5 g, 91%). [M+H]⁺=253.1.

### Step 8: (6-((5-bromo-2-chloropyrimidin-4-yl)amino)-3-fluoro-2-methylquinolin-5-yl)dimethylphosphine oxide

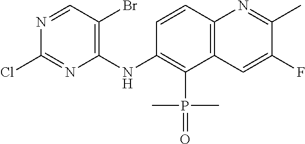 [SMILES: Cc1nc2ccc(Nc3nc(Cl)ncc3Br)c(P(C)(C)=O)c2cc1F]

To a solution of (6-amino-3-fluoro-2-methylquinolin-5-yl)dimethylphosphine oxide (2.5 g, 9.9 mmol) and 5-bromo-2,4-dichloropyrimidine (6.7 g, 29.7 mmol) in n-BuOH (100 mL) was added DIEA(3.8 g, 29.7 mmol) at room temperature. The resulting mixture was stirred at 120° C. overnight. The reaction was concentrated to afford the crude residue, which was purified by silica gel column chromatography (DCM: MeOH=100: 0˜15: 1 gradient elution) to give the desired product (2.9 g, 66%). [M+H]⁺=443.1.

### Step 9: (6-((5-bromo-2-((5-ethyl-2-methoxy-4-(4-(piperazin-1-yl)piperidin-1-yl)phenyl)amino)pyrimidin-4-yl)amino)-3-fluoro-2-methylquinolin-5-yl)dimethylphosphine oxide

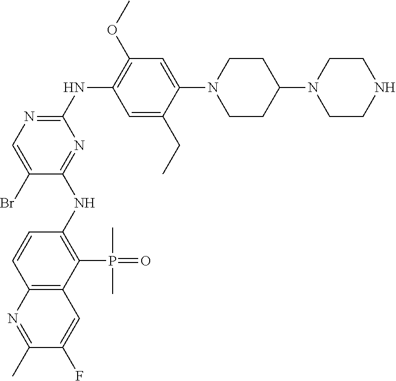 [SMILES: CCc1cc(Nc2ncc(Br)c(Nc3ccc4nc(C)c(F)cc4c3P(C)(C)=O)n2)c(OC)cc1N1CCC(N2CCNCC2)CC1]

To a solution of (6-((5-bromo-2-chloropyrimidin-4-yl)amino)-3-fluoro-2-methylquinolin-5-yl)dimethylphosphine oxide (1.7 g, 3.8 mmol) and tert-butyl 4-(1-(4-amino-2-ethyl-5-methoxyphenyl)piperidin-4-yl)piperazine-1-carboxylate (1.6 g, 3.8 mmol) in n-BuOH (100 mL) was added Ts-OH (2.0 g, 11.4 mmol) at room temperature. The resulting mixture was stirred at 100° C. overnight. The reaction was concentrated and basified with 0.5N NaOH (50 mL). The resulting mixture was extracted with DCM (3×80 mL). The combined organic layers were dried over anhydrous Na₂SO₄, filtered and evaporated in vacuum to afford the crude residue, which was purified by silica gel column chromatography (DCM: MeOH=100: 0˜5: 1 gradient elution) to give the desired product (960 mg, 35%). [M+H]⁺=725.2.

### Step 10: methyl (R)-1-(4-bromo-3,5-difluorophenyl)pyrrolidine-3-carboxylate

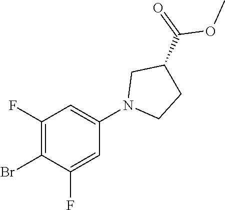 [SMILES: COC(=O)[C@@H]1CCN(c2cc(F)c(Br)c(F)c2)C1]

To the solution of 2-bromo-1,3-difluoro-5-iodobenzene (15 g, 47 mmol), methyl (R)-pyrrolidine-3-carboxylate hydrochloride (8.56 g, 51.7 mmol) and K₃PO₄ (20 g, 94 mmol) in 250 mL DMSO, were added CuI (893 mg, 4.7 mmol) and L-proline (1 g, 9.4 mmol). The mixture was stirred at 80° C. for 16 hours. After LCMS showed the reaction was completed, the mixture was diluted with water and extracted by EtOAc. The organic layer was washed with brine, dried over anhydrous Na₂SO₄, filtered and concentrated under reduced pressure. The resulting mixture was purified by silica column chromatography (PE:EA=50:1-30:1) to afford the product (4.9 g, 32.5%). [M+H]⁺=320.1.

### Step 11: methyl (R)-1-(4-(2,6-bis(benzyloxy)pyridin-3-yl)-3,5-difluorophenyl)pyrrolidine-3-carboxylate

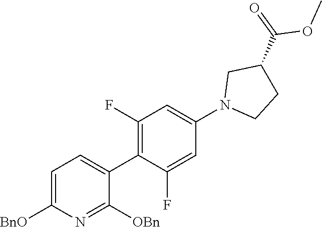 [SMILES: COC(=O)[C@@H]1CCN(c2cc(F)c(-c3ccc(OCc4ccccc4)nc3OCc3ccccc3)c(F)c2)C1]

To the solution of methyl (R)-1-(4-bromo-3,5-difluorophenyl)pyrrolidine-3-carboxylate (4.9 g, 15.3 mmol), 2,6-bis(benzyloxy)-3-(4,4,5,5-tetramethyl-1,3,2-dioxaborolan-2-yl)pyridine (6.7 g, 16 mmol) and CsF (4.6 g, 30.6 mmol) in 150 mL DMF and 15 mL water, was added Pd(dtbpf)Cl₂ (498 mg, 0.8 mmol). The mixture was stirred at 80° C. for 4 hours. After LCMS showed the reaction was completed, the mixture was diluted with water and extracted with EtOAc. The organic layer was washed with brine, dried over anhydrous Na₂SO₄, filtered and concentrated under reduced pressure.

The resulting mixture was purified by combi-flash (EA:PE=0-12%) to afford the product (7.9 g, 97.4%). [M+H]⁺=531.30.

### Step 12: (R)-1-(4-(2,6-bis(benzyloxy)pyridin-3-yl)-3,5-difluorophenyl)pyrrolidine-3-carboxylic acid

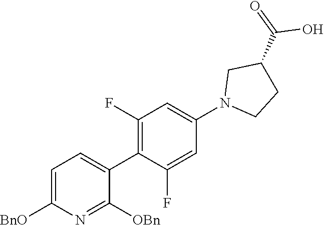 [SMILES: O=C(O)[C@@H]1CCN(c2cc(F)c(-c3ccc(OCc4ccccc4)nc3OCc3ccccc3)c(F)c2)C1]

To the solution of methyl (R)-1-(4-(2,6-bis(benzyloxy)pyridin-3-yl)-3,5-difluorophenyl)pyrrolidine-3-carboxylate (7.9 g, 14.9 mmol) in 100 mL THF and 20 mL water, LiOH (394 mg, 16.4 mmol) in 10 mL water was added dropwise at room temperature. The mixture was stirred at room temperature for 15 minutes. After TLC showed the reaction was completed, the mixture was concentrated in vacuum at room temperature. The residue was diluted with water and adjust pH<5 with 1 N HCl. The liquid was extracted with EtOAc. The organic phase was dried over anhydrous Na₂SO₄, filtered and concentrated in vacuum to afford the product (7.4 g, 96.0%). [M+H]⁺=517.1.

### Step 13: (R)-1-(4-((R)-2,6-dioxopiperidin-3-yl)-3,5-difluorophenyl)pyrrolidine-3-carboxylic acid

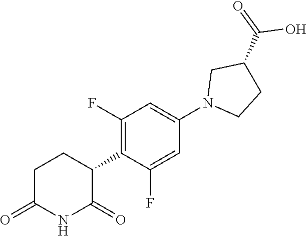 [SMILES: O=C1CC[C@H](c2c(F)cc(N3CC[C@@H](C(=O)O)C3)cc2F)C(=O)N1]

To the solution of (R)-1-(4-(2,6-bis(benzyloxy)pyridin-3-yl)-3,5-difluorophenyl)pyrrolidine-3-carboxylic acid (7.4 g, 14.3 mmol) in 50 mL DCM and 250 mL iPrOH, Pd/C (7.4 g, 10 wt. %, wet) was added. The mixture was stirred at 40° C. for 16 hours under hydrogen atmosphere (balloon). After LCMS showed the reaction was completed, the mixture was cooled to room temperature and filtered by celite directly. The filtrate was concentrated in vacuum to afford the crude product which was purified by SFC (IH (3*25 cm, 5 um), 13% EtOH/87% CO₂, 100 bar, 100 ml/min) and the title compound corresponded to peak A @ 0.655 min/254 nm (1.6 g, 34%). [M+H]⁺=339.2.

### Step 14: (R)-3-(4-((R)-3-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

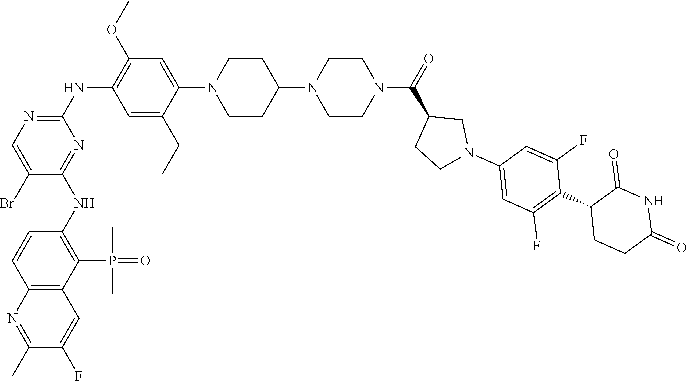 [SMILES: CCc1cc(Nc2ncc(Br)c(Nc3ccc4nc(C)c(F)cc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

To a solution of (6-((5-bromo-2-((5-ethyl-2-methoxy-4-(4-(piperazin-1-yl)piperidin-1-yl)phenyl)amino)pyrimidin-4-yl)amino)-3-fluoro-2-methylquinolin-5-yl)dimethylphosphine oxide (450 mg, 0.62 mmol), (R)-1-(4-((R)-2,6-dioxopiperidin-3-yl)-3,5-difluorophenyl)pyrrolidine-3-carboxylic acid (230 mg, 0.68 mmol) and DIEA (162 mg, 1.24 mmol) in DCM (30 mL) was added T3P (788 mg, 1.24 mmol) dropwise at room temperature. The resulting mixture was stirred at room temperature for 1 hour. The reaction was quenched with water (50 mL) and extracted with DCM (2×60 mL). The combined organic layers were dried over anhydrous Na₂SO₄, filtered and evaporated in vacuum to afford the crude residue, which was purified by silica gel column chromatography (DCM: MeOH=100: 0˜10: 1 gradient elution) to give an impure product, which was further purified with prep-HPLC (C-18 column chromatography (0.1% FA in water: acetonitrile=90: 10˜ 60: 40 gradient elution) to give the desired product (302 mg, 47%). ¹H NMR (500 MHz, DMSO) δ 11.30 (s, 1H), 10.84 (s, 1H), 8.63 (d, J=12.6 Hz, 1H), 8.23 (s, 2H), 7.95 (d, J=9.2 Hz, 2H), 7.33 (s, 1H), 6.72 (s, 1H), 6.23 (d, J=12.1 Hz, 2H), 4.02 (dd, J=12.7, 5.0 Hz, 1H), 3.75 (s, 3H), 3.60-3.42 (m, 6H), 3.35 (s, 2H), 3.26 (s, 3H), 2.92 (d, J=10.7 Hz, 2H), 2.83-2.73 (m, 1H), 2.64 (d, J=2.4 Hz, 5H), 2.56 (s, 3H), 2.37 (d, J=3.6 Hz, 1H), 2.25-2.05 (m, 5H), 1.95 (d, J=13.4 Hz, 7H), 1.82 (d, J=11.0 Hz, 2H), 1.62-1.51 (m, 2H), 0.71 (s, 3H); [M+H]⁺=1045.5.

### Example 489: (R)-3-(4-((R)-3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl) piperazine-1-carbonyl)pyrrolidin-1-yl)-2,6-difluorophenyl)piperidine-2,6-dione

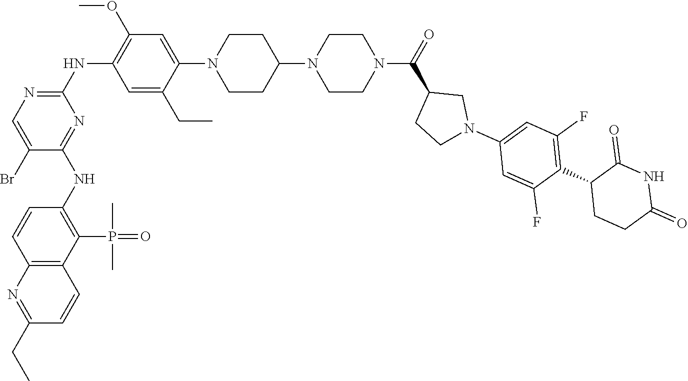 [SMILES: CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1]

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (1.15 g, 1.60 mmol), (R)-1-(4-((R)-2,6-dioxopiperidin-3-yl)-3,5-difluorophenyl)pyrrolidine-3-carboxylic acid (595 mg, 1.76 mmol), DIEA (411 mg, 3.19 mmol) and T3P (763 mg, 2.4 mmol) in DCM (8 mL) was stirred in a flask at room temperature for 0.5 h. Then the mixture was evaporated in vacuum to afford the crude product, which was purified with prep-HPLC chromatography (0.1% FA in water: acetonitrile=90:10˜ 50:50 gradient elution) to give the product (800 mg, 48.3%).

¹H NMR (500 MHz, DMSO) δ 11.80 (s, 1H), 10.84 (s, 1H), 8.56 (d, J=8.9 Hz, 1H), 8.27 (s, 1H), 8.21 (s, 1H), 8.00 (s, 1H), 7.88 (d, J=9.1 Hz, 1H), 7.44 (d, J=8.9 Hz, 1H), 7.34 (s, 1H), 6.75 (s, 1H), 6.23 (d, J=12.2 Hz, 2H), 4.02 (dd, J=12.3, 4.7 Hz, 1H), 3.76 (s, 3H), 3.65-3.41 (m, 7H), 3.31-3.23 (m, 4H), 3.01-2.88 (m, 4H), 2.84-2.74 (m, 1H), 2.68 (t, J=11.1 Hz, 2H), 2.59-2.55 (m, 3H), 2.39-2.35 (m, 1H), 2.31-2.28 (m, 2H), 2.19-2.06 (m, 3H), 1.98 (d, J=13.3 Hz, 6H), 1.96-1.91 (m, 1H), 1.84 (d, J=10.1 Hz, 2H), 1.57 (d, J=9.9 Hz, 2H), 1.32 (t, J=7.5 Hz, 3H), 0.77 (s, 3H). [M+H]⁺=1041.7

### Example 441: 3-(7-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-26-dione

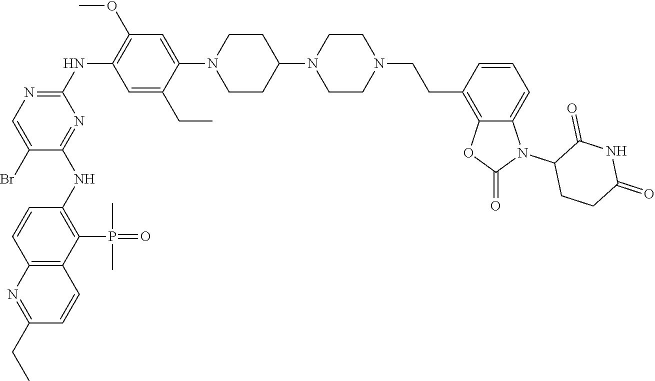 [SMILES: CCc1ccc2c(P(C)(C)=O)c(Nc3nc(Nc4cc(CC)c(N5CCC(N6CCN(CCc7cccc8c7oc(=O)n8C7CCC(=O)NC7=O)CC6)CC5)cc4OC)ncc3Br)ccc2n1]

The title compound (19 mg, 20%) was prepared in a manner similar to that in Example 492. ¹H NMR (500 MHz, DMSO) δ 11.78 (s, 1H), 11.22 (s, 1H), 8.57 (d, J=8.9 Hz, 1H), 8.27 (s, 1H), 8.21 (s, 1H), 8.00 (s, 1H), 7.88 (d, J=9.4 Hz, 1H), 7.44 (d, J=8.9 Hz, 1H), 7.35 (s, 1H), 7.19-7.02 (m, 3H), 6.75 (s, 1H), 3.76 (s, 3H), 3.01-2.82 (m, 9H), 2.79-2.53 (m, 14H), 2.29 (d, J=6.7 Hz, 2H), 2.20-2.10 (m, 1H), 1.98 (d, J=13.3 Hz, 6H), 1.90 (d, J=9.9 Hz, 2H), 1.59 (d, J=9.4 Hz, 2H), 1.32 (t, J=7.6 Hz, 3H), 0.83-0.71 (s, 3H); [M+H]⁺=993.5.

### Example 442: 3-(7-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-5-methyl-2-oxobenzo[d]oxazol-3(2H)-yl)piperidine-2,6-dione

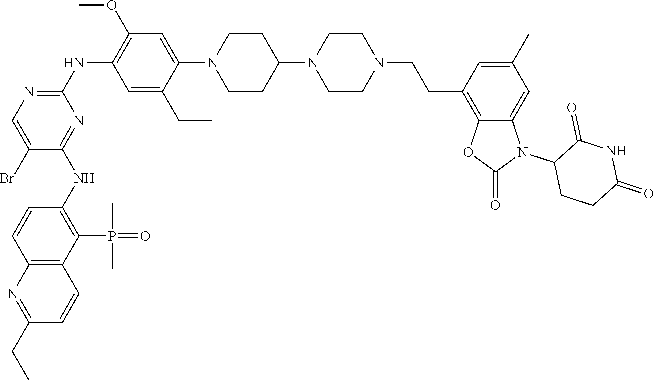 [SMILES: CCc1ccc2c(P(C)(C)=O)c(Nc3nc(Nc4cc(CC)c(N5CCC(N6CCN(CCc7cc(C)cc8c7oc(=O)n8C7CCC(=O)NC7=O)CC6)CC5)cc4OC)ncc3Br)ccc2n1]

The title compound (35 mg, 42%) was prepared in a manner similar to that in Example 492. ¹H NMR (500 MHz, DMSO) δ 11.79 (s, 1H), 11.19 (s, 1H), 8.56 (d, J=9.0 Hz, 1H), 8.27 (s, 1H), 8.21 (s, 1H), 8.01 (s, 1H), 7.87 (d, J=9.3 Hz, 1H), 7.44 (d, J=8.9 Hz, 1H), 7.33 (s, 1H), 6.95 (s, 1H), 6.87 (s, 1H), 6.75 (s, 1H), 5.33 (dd, J=13.1, 5.3 Hz, 1H), 3.76 (s, 3H), 2.92 (dt, J=16.1, 8.0 Hz, 5H), 2.85-2.78 (m, 2H), 2.73-2.60 (m, 5H), 2.55 (dd, J=10.1, 5.0 Hz, 5H), 2.49-2.40 (m, 4H), 2.31 (s, 6H), 2.17-2.07 (m, 1H), 1.98 (d, J=13.3 Hz, 6H), 1.84 (d, J=10.4 Hz, 2H), 1.60-1.47 (m, 2H), 1.32 (t, J=7.6 Hz, 3H), 0.77 (s, 3H). [M+H]⁺=1007.4.

### Example 443: 3-(7-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-5-fluoro-2-oxobenzo[d]oxazol-3(2H)-yl)piperidine-2,6-dione

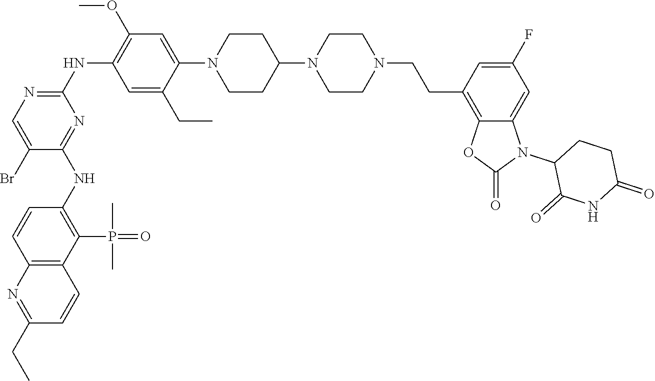 [SMILES: CCc1ccc2c(P(C)(C)=O)c(Nc3nc(Nc4cc(CC)c(N5CCC(N6CCN(CCc7cc(F)cc8c7oc(=O)n8C7CCC(=O)NC7=O)CC6)CC5)cc4OC)ncc3Br)ccc2n1]

The title compound (23.52 mg, 38%) was prepared in a manner similar to that in Example 492.

¹H NMR (500 MHz, DMSO) δ 11.80 (s, 1H), 11.21 (s, 1H), 8.55 (d, J=9.0 Hz, 1H), 8.26 (d, J=13.4 Hz, 1H), 8.21 (s, 1H), 8.01 (s, 1H), 7.87 (d, J=9.0 Hz, 1H), 7.44 (d, J=8.9 Hz, 1H), 7.32 (s, 1H), 7.17 (dd, J=8.5, 2.5 Hz, 1H), 6.97 (dd, J=10.7, 2.4 Hz, 1H), 6.75 (s, 1H), 5.37-5.34 (m, 1H), 3.76 (s, 3H), 2.94-2.92 (m, 4H), 2.88-2.84 (m, 3H), 2.75-2.64 (m, 4H), 2.61-2.53 (m, 5H), 2.32 (m, 4H), 2.18-2.08 (m, 1H), 1.99-1.94 (m, 7H), 1.86-1.82 (m, 2H), 1.64-1.41 (m, 2H), 1.41-1.20 (m, 3H), 0.77 (s, 3H). [M+H]⁺=1011.6.

### Example 444: 3-(7-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-6-fluoro-2-oxobenzo[d]oxazol-3(2H)-yl)piperidine-2,6-dione

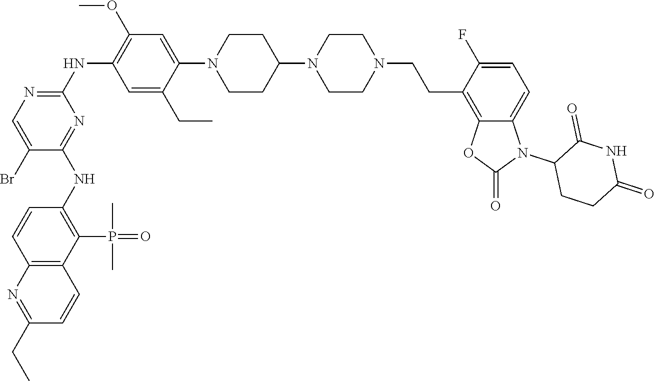 [SMILES: CCc1ccc2c(P(C)(C)=O)c(Nc3nc(Nc4cc(CC)c(N5CCC(N6CCN(CCc7c(F)ccc8c7oc(=O)n8C7CCC(=O)NC7=O)CC6)CC5)cc4OC)ncc3Br)ccc2n1]

The title compound (15 mg, 16%) was prepared in a manner similar to that in Example 492.

¹H NMR (500 MHz, DMSO) δ 11.80 (s, 1H), 11.22 (s, 1H), 8.55 (d, J=9.0 Hz, 1H), 8.26 (s, 1H), 8.21 (s, 1H), 8.00 (s, 1H), 7.87 (d, J=9.4 Hz, 1H), 7.44 (d, J=8.9 Hz, 1H), 7.32 (s, 1H), 7.16-7.12 (m, 1H), 7.07 (d, J=8.6 Hz, 1H), 6.75 (s, 1H), 5.37 (dd, J=13.1, 5.3 Hz, 1H), 3.76 (s, 3H), 2.98-2.80 (m, 8H), 2.73-2.62 (m, 5H), 2.58-2.51 (m, 8H), 2.33-2.23 (m, 3H), 2.20-2.09 (m, 1H), 1.98 (d, J=13.3 Hz, 6H), 1.89-1.78 (m, 2H), 1.60-1.44 (m, 2H), 1.32 (t, J=7.6 Hz, 3H), 0.83-0.71 (m, 3H); [M+H]⁺=1011.5.

### Example 445: 3-(7-((R)-3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazine-1-carbonyl)pyrrolidin-1-yl)-2-oxobenzo[d]oxazol-3(2H)-yl)piperidine-2,6-dione

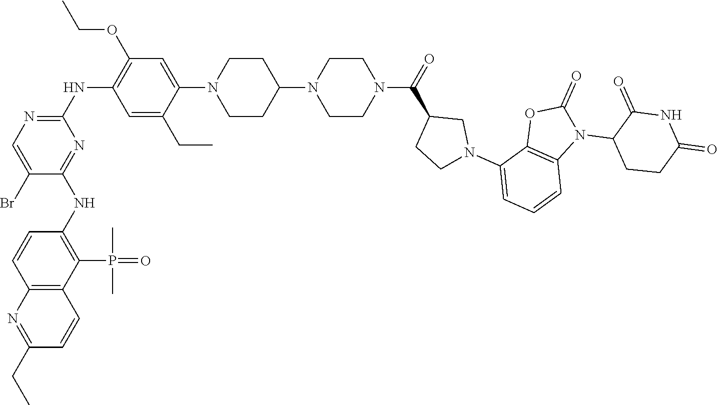 [SMILES: CCOc1cc(N2CCC(N3CCN(C(=O)[C@@H]4CCN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound (20 mg, 60.5%) was prepared in a manner similar to that in Example 447. ¹H NMR (500 MHz, DMSO) δ 11.72 (s, 1H), 11.19 (s, 1H), 8.60 (d, J=8.5 Hz, 1H), 8.22 (s, 2H), 7.96-7.81 (m, 2H), 7.48-7.36 (m, 2H), 7.01 (d, J=8.1 Hz, 1H), 6.71 (s, 1H), 6.53 (d, J=7.8 Hz, 1H), 6.40 (d, J=8.5 Hz, 1H), 5.30 (dd, J=12.9, 5.4 Hz, 1H), 4.01 (t, J=6.9 Hz, 2H), 3.67 (s, 1H), 3.60 (dd, J=16.4, 6.7 Hz, 3H), 3.54-3.45 (m, 6H), 2.93 (dd, J=15.0, 7.4 Hz, 5H), 2.65 (dd, J=20.2, 7.2 Hz, 4H), 2.55 (d, J=11.2 Hz, 2H), 2.35 (d, J=11.9 Hz, 1H), 2.25 (d, J=6.7 Hz, 2H), 2.20-2.03 (m, 4H), 1.98 (d, J=13.3 Hz, 6H), 1.83 (d, J=10.1 Hz, 2H), 1.57 (s, 2H), 1.32 (t, J=7.6 Hz, 3H), 1.26 (t, J=6.9 Hz, 3H), 0.71 (s, 3H). [M+H]⁺=1076.4.

### Example 446: 3-(7-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)azetidin-1-yl)-2-oxobenzo[d]oxazol-3(2H)-yl)piperidine-2,6-dione

### Step 1: 3-(2,6-bis(benzyloxy)pyridin-3-yl)-7-bromobenzo[d]oxazol-2(3H)-one

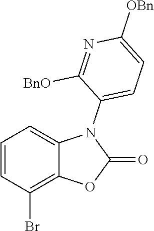 [SMILES: O=c1oc2c(Br)cccc2n1-c1ccc(OCc2ccccc2)nc1OCc1ccccc1]

A mixture of 7-bromo-3H-1,3-benzoxazol-2-one (6 g, 28.03 mmol), Cu(OAc)₂ (5.09 g, 28.03 mmol), pyridine (6.65 mL, 84.1 mmol), 2,6-bis(benzyloxy)-3-(4,4,5,5-tetramethyl-1,3,2-dioxaborolan-2-yl)pyridine (11.7 g, 28.03 mmol) and 4A MS (6 g) in 1,4-dioxane (120 mL) was stirred overnight at 80° C. under oxygen atmosphere. The mixture was allowed to cool down to room temperature. The resulting mixture was filtered, and the solid was washed with EtOAc (500 mL). The combined organic filtrates were washed with brine (500 mL×3), dried over anhydrous Na₂SO₄, filtered and concentrated under reduced pressure. Purification by reverse flash chromatography (DCM in PE, 10% to 50% gradient in 30 min) afforded the product (9.6 g, 68.03%). [M+H]⁺=503.2.

### Step 2: benzyl 1-(3-(2,6-bis(benzyloxy)pyridin-3-yl)-2-oxo-2,3-dihydrobenzo[d]oxazol-7-yl)azetidine-3-carboxylate

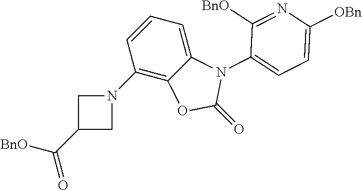 [SMILES: O=C(OCc1ccccc1)C1CN(c2cccc3c2oc(=O)n3-c2ccc(OCc3ccccc3)nc2OCc2ccccc2)C1]

A mixture of 3-(2,6-bis(benzyloxy)pyridin-3-yl)-7-bromobenzo[d]oxazol-2(3H)-one (1.1 g, 2.18 mmol), benzyl 1-(2,2,2-trifluoroacetyl)azetidine-3-carboxylate (0.94 g, 3.277 mmol), Cs₂CO₃ (2.14 g, 6.55 mmol), Pd₂(dba)₃ (0.20 g, 0.22 mmol) and RuPhos (0.10 g, 0.22 mmol) in 1,4-dioxane (20 mL) was stirred for 2 h at 100° C. under nitrogen atmosphere. The mixture was allowed to cool down to room temperature and diluted with EtOAc (200 mL), which was washed with brine (200 mL×3), dried over anhydrous Na₂SO₄, filtered, and concentrated under reduced pressure. Purification by silica gel column chromatography (PE/EA 1:1) afforded the product (620 mg, 46.23%). [M+H]⁺=614.3.

### Step 3: 1-(3-(2,6-dioxopiperidin-3-yl)-2-oxo-2,3-dihydrobenzo[d]oxazol-7-yl)azetidine-3-carboxylic acid

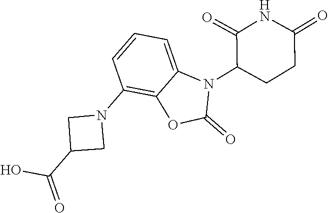 [SMILES: O=C1CCC(n2c(=O)oc3c(N4CC(C(=O)O)C4)cccc32)C(=O)N1]

A mixture of_benzyl 1-(3-(2,6-bis(benzyloxy)pyridin-3-yl)-2-oxo-2,3-dihydrobenzo[d]oxazol-7-yl)azetidine-3-carboxylate (600 mg, 0.98 mmol) and Pd/C (10 wt. %, wet, 600 mg) in THF (10 mL) was stirred for 5 h at 50° C. under hydrogen atmosphere. The mixture was allowed to cool down to room temperature. The resulting mixture was filtered, and the solid was washed with THF. The filtrate was concentrated under reduced pressure to afford the product (241 mg, 71.38%), which was used without further purification. [M+H]⁺=346.2.

### Step 4: 3-(7-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)azetidin-1-yl)-2-oxobenzo[d]oxazol-3(2H)-yl)piperidine-2,6-dione

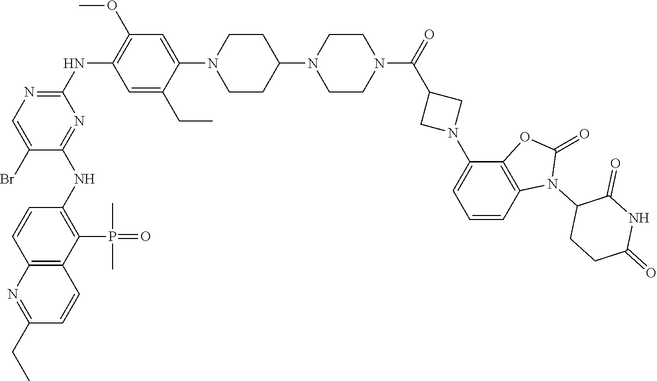 [SMILES: CCc1ccc2c(P(C)(C)=O)c(Nc3nc(Nc4cc(CC)c(N5CCC(N6CCN(C(=O)C7CN(c8cccc9c8oc(=O)n9C8CCC(=O)NC8=O)C7)CC6)CC5)cc4OC)ncc3Br)ccc2n1]

The title compound (45 mg, 65.2%) was prepared in a manner similar to that in Example 447 step 4 from (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide and 1-(3-(2,6-dioxopiperidin-3-yl)-2-oxo-2,3-dihydrobenzo[d]oxazol-7-yl)azetidine-3-carboxylic acid.

¹H NMR (500 MHz, DMSO) δ 11.79 (s, 1H), 11.19 (s, 1H), 8.56 (d, J=9.1 Hz, 1H), 8.29 (s, 1H), 8.21 (s, 1H), 8.00 (s, 1H), 7.87 (d, J=9.2 Hz, 1H), 7.44 (d, J=8.9 Hz, 1H), 7.33 (s, 1H), 7.03 (t, J=8.0 Hz, 1H), 6.75 (s, 1H), 6.62 (d, J=8.0 Hz, 1H), 6.31 (d, J=8.3 Hz, 1H), 5.30 (dd, J=12.9, 5.2 Hz, 1H), 4.22 (t, J=7.7 Hz, 2H), 4.08 (t, J=6.8 Hz, 2H), 3.93-3.83 (m, 1H), 3.76 (s, 3H), 3.49 (s, 3H), 2.93 (s, 6H), 2.61-2.55 (m, 6H), 2.58 (s, 3H), 2.43-2.25 (m, 6H), 2.13 (d, J=5.1 Hz, 1H), 1.98 (d, J=13.3 Hz, 6H), 1.83 (d, J=10.9 Hz, 2H), 1.57 (d, J=9.2 Hz, 2H), 1.32 (t, J=7.6 Hz, 3H), 0.77 (s, 3H). [M+H]⁺=1048.3

### Example 447: 3-(7-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)azetidin-1-yl)-5-fluoro-2-oxobenzo[d]oxazol-3(2H)-yl)piperidine-2,6-dione

### Step 1: methyl 1-(3-(2,6-bis(benzyloxy)pyridin-3-yl)-5-fluoro-2-oxo-2,3-dihydrobenzo[d]oxazol-7-yl)azetidine-3-carboxylate

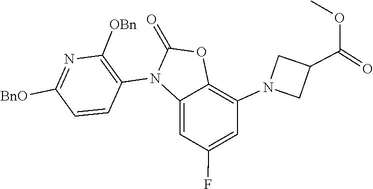 [SMILES: COC(=O)C1CN(c2cc(F)cc3c2oc(=O)n3-c2ccc(OCc3ccccc3)nc2OCc2ccccc2)C1]

A mixture of 3-(2,6-bis(benzyloxy)pyridin-3-yl)-7-bromo-5-fluorobenzo[d]oxazol-2(3H)-one (1 g, 1.92 mmol, obtained by the similar way to example 446), methyl azetidine-3-carboxylate hydrochloride (435 mg, 2.88 mmol), Pd₂(dba)₃ (176 mg, 0.192 mmol), Xantphos (222 mg, 0.384 mmol), and Cs₂CO₃ (1.25 g, 3.84 mmol) in dioxane (30 mL) was stirred at 100° C. for 18 hrs under nitrogen atmosphere. After cooling to rt, the reaction was diluted with EA (60 mL) and then washed with brine (20 mL×3), dried over Na₂SO₄, filtered and concentrated under vacuum. The residue was purified by silica gel column (PE:EA=1:1) to afford methyl 1-(3-(2,6-bis(benzyloxy)pyridin-3-yl)-5-fluoro-2-oxo-2,3-dihydrobenzo[d]oxazol-7-yl)azetidine-3-carboxylate (560 mg, 52.4%). [M+H]⁺=556.3.

### Step 2: 1-(3-(2,6-bis(benzyloxy)pyridin-3-yl)-5-fluoro-2-oxo-2,3-dihydrobenzo[d]oxazol-7-yl)azetidine-3-carboxylic acid

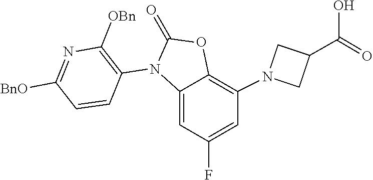 [SMILES: O=C(O)C1CN(c2cc(F)cc3c2oc(=O)n3-c2ccc(OCc3ccccc3)nc2OCc2ccccc2)C1]

To a solution of methyl 1-(3-(2,6-bis(benzyloxy)pyridin-3-yl)-5-fluoro-2-oxo-2,3-dihydrobenzo[d]oxazol-7-yl)azetidine-3-carboxylate (500 mg, 0.99 mmol) in THF (30 mL) and H₂O (8 mL) was added LiOH H₂O (210 mg, 5 mmol). The reaction was stirred at rt for 3 hr, before HCl (1 N) was added to PH=5-6. The layers were separated and the aqueous layer was extracted with EA (30 mL×3). The combined organic layers were washed with brine (20 mL), dried over Na₂SO₄, filtered and concentrated under vacuum to afford 1-(3-(2,6-bis(benzyloxy)pyridin-3-yl)-5-fluoro-2-oxo-2,3-dihydrobenzo[d]oxazol-7-yl)azetidine-3-carboxylic acid (495 mg, 92.3%). [M+H]⁺=542.3.

### Step 3: 1-(3-(2,6-dioxopiperidin-3-yl)-5-fluoro-2-oxo-2,3-dihydrobenzo[d]oxazol-7-yl)azetidine-3-carboxylic acid

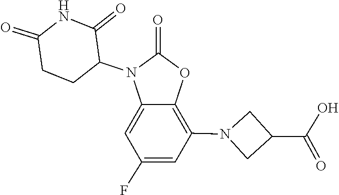 [SMILES: O=C1CCC(n2c(=O)oc3c(N4CC(C(=O)O)C4)cc(F)cc32)C(=O)N1]

The title compound (260 mg, 89%) was prepared in a manner similar to that in Example 458 step 9 from 1-(3-(2,6-bis(benzyloxy)pyridin-3-yl)-5-fluoro-2-oxo-2,3-dihydrobenzo[d]oxazol-7-yl)azetidine-3-carboxylic acid. [M+H]⁺=364.3.

### Step 4: 3-(7-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)azetidin-1-yl)-5-fluoro-2-oxobenzo[d]oxazol-3(2H)-yl)piperidine-2,6-dione

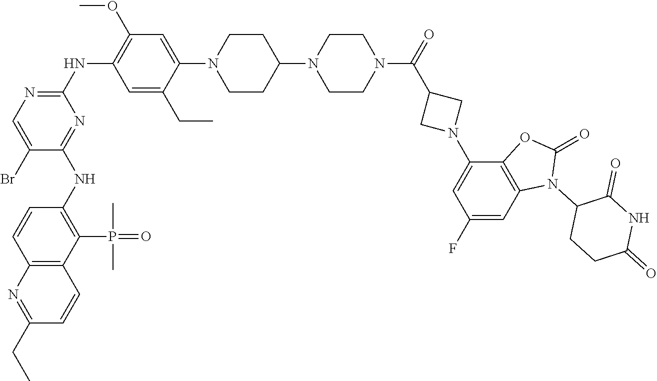 [SMILES: CCc1ccc2c(P(C)(C)=O)c(Nc3nc(Nc4cc(CC)c(N5CCC(N6CCN(C(=O)C7CN(c8cc(F)cc9c8oc(=O)n9C8CCC(=O)NC8=O)C7)CC6)CC5)cc4OC)ncc3Br)ccc2n1]

To a solution of (6-((5-bromo-2-((2-methoxy-5-ethyl-4-(4-(piperazin-1-yl)piperidin-1-yl)phenyl)amino)pyrimidin-4-yl)amino)-2-ethylquinolin-5-yl)dimethylphosphine oxide (50 mg, 0.069 mmol), 1-(3-(2,6-dioxopiperidin-3-yl)-5-fluoro-2-oxo-2,3-dihydrobenzo[d]oxazol-7-yl)azetidine-3-carboxylic acid (30 mg, 0.083 mmol) in DMF (3 mL) was added HATU (31.5 mg, 0.083 mmol) and DIEA (22 mg, 0.166 mmol). After stirring at r. t. for 30 min, the reaction was quenched with water (6 mL), and the resulting mixture was extracted with DCM (10 mL×3). The combined organic layers were washed with brine (30 mL×3), dried over Na₂SO₄, filtered and concentrated in vacuum. The residue was purified with prep-HPLC chromatography (0.1% FA in water: acetonitrile=90:10˜ 50:50 gradient elution) to give the product (12.15 mg, 23%).

¹H NMR (500 MHz, DMSO) δ 11.79 (s, 1H), 11.19 (s, 1H), 8.56 (d, J=8.9 Hz, 1H), 8.27 (s, 1H), 8.21 (s, 1H), 8.00 (s, 1H), 7.88 (d, J=9.1 Hz, 1H), 7.44 (d, J=8.9 Hz, 1H), 7.34 (s, 1H), 6.75 (s, 1H), 6.61 (dd, J=8.5, 2.3 Hz, 1H), 6.17 (dd, J=11.9, 2.3 Hz, 1H), 5.29 (dd, J=12.8, 5.1 Hz, 1H), 4.24 (t, J=8.0 Hz, 2H), 4.11 (t, J=6.9 Hz, 2H), 3.93-3.83 (m, 1H), 3.76 (s, 3H), 3.49 (s, 2H), 3.32-3.31 (m, 3H), 3.03-2.87 (m, 4H), 2.87-2.82 (m, 1H), 2.75-2.60 (m, 5H), 2.54 (s, 2H), 2.37 (d, J=10.9 Hz, 1H), 2.29 (d, J=7.1 Hz, 2H), 2.19-2.06 (m, 1H), 2.00 (s, 3H), 1.98 (s, 3H), 1.84 (d, J=9.3 Hz, 2H), 1.57 (d, J=9.6 Hz, 2H), 1.33-1.31 (m, 3H), 0.77 (s, 3H). [M+H]⁺=1066.7.

### Example 448: 3-(7-(3-((4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)methyl)azetidin-1-yl)-2-oxobenzo[d]oxazol-3(2H)-yl)piperidine-2,6-dione

### Step 1: 7-(3-((benzyloxy)methyl)azetidin-1-yl)-3-(2,6-bis(benzyloxy)pyridin-3-yl)benzo[d]oxazol-2(3H)-one

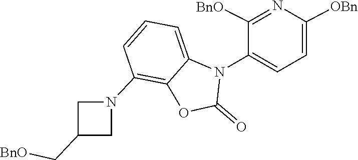 [SMILES: O=c1oc2c(N3CC(COCc4ccccc4)C3)cccc2n1-c1ccc(OCc2ccccc2)nc1OCc1ccccc1]

To a solution of 3-(2,6-bis(benzyloxy)pyridin-3-yl)-7-bromobenzo[d]oxazol-2(3H)-one (3 g, 5.01 mmol), 3-((benzyloxy)methyl)azetidine (2 g, 6.90 mmol) in dry 1,4-dioxane (50 mL), were added tris(dibenzylideneacetone)dipalladium (600 mg, 0.66 mmol), Ruphos (600 mg, 1.29 mmol), and cesium carbonate (6.00 g, 18.4 mmol). The mixture was stirred overnight at 100° C. under nitrogen atmosphere. After cooled to room temperature, the mixture was diluted with ethyl acetate (300 mL). The combined organic layers were washed with brine (300 mL×3), dried over anhydrous Na₂SO₄. After filtration, the filtrate was concentrated under reduced pressure. The residue was purified by column chromatography (petroleum ether/ethyl acetate 15%˜20%) to afford the target product (1.42 g, 46.7%). [M+H]⁺=600.1.

### Step 2: 3-(7-(3-(hydroxymethyl)azetidin-1-yl)-2-oxobenzo[d]oxazol-3(2H)-yl)piperidine-2,6-dione

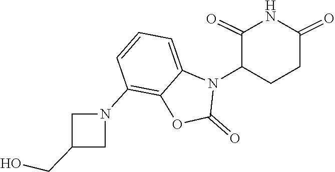 [SMILES: O=C1CCC(n2c(=O)oc3c(N4CC(CO)C4)cccc32)C(=O)N1]

To a solution of 7-(3-((benzyloxy)methyl)azetidin-1-yl)-3-(2,6-bis(benzyloxy)pyridin-3-yl)benzo[d]oxazol-2(3H)-one (1.4 g, 2.34 mmol) in tetrahydrofuran (40 mL), was added Pd/C (10 wt. %, wet, 1.5 g). The flask was evacuated and backfilled with hydrogen gas for 5 times, and stirred overnight under hydrogen atmosphere at 50° C. After cooled to rt, the resulting mixture was filtered, and the solid was washed with THF. The filtrate was concentrated under reduced pressure. The crude solid was washed by petroleum ether and dried under vacuum to afford the target product (650 mg, 83.9%). [M+H]⁺=332.1.

### Step 3: 1-(3-(2,6-dioxopiperidin-3-yl)-2-oxo-2,3-dihydrobenzo[d]oxazol-7-yl)azetidine-3-carbaldehyde

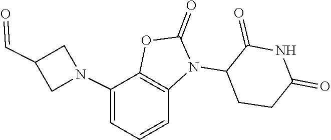 [SMILES: O=CC1CN(c2cccc3c2oc(=O)n3C2CCC(=O)NC2=O)C1]

A mixture of 3-(7-(3-(hydroxymethyl)azetidin-1-yl)-2-oxobenzo[d]oxazol-3(2H)-yl)piperidine-2,6-dione (100 mg, 0.30 mmol) and IBX (132 mg, 0.47 mmol) in DMSO (10 mL) was stirred in a flask at room temperature overnight. The reaction was quenched with water and the mixture was extracted with EtOAc (20 mL×3). The combined organic layers were washed with sat. aq. NaCl (30 mL×3), sat. aq. NaHCO₃(30 mL×2), dried over anhydrous Na₂SO₄, filtered and concentrated under vacuum to afford the product (70 mg, 70.1%). [M+H]⁺=330.1.

### Step 4: 3-(7-(3-((4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)methyl)azetidin-1-yl)-2-oxobenzo[d]oxazol-3(2H)-yl)piperidine-2,6-dione

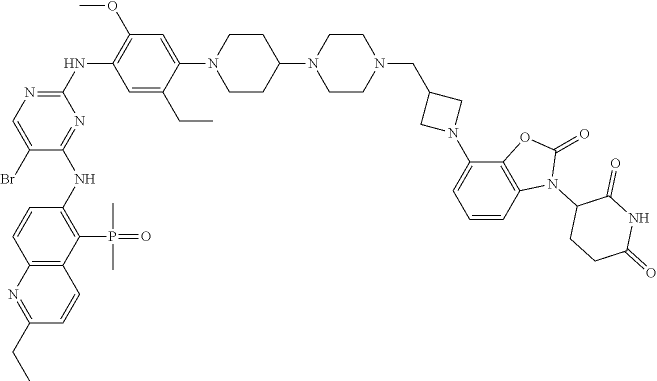 [SMILES: CCc1ccc2c(P(C)(C)=O)c(Nc3nc(Nc4cc(CC)c(N5CCC(N6CCN(CC7CN(c8cccc9c8oc(=O)n9C8CCC(=O)NC8=O)C7)CC6)CC5)cc4OC)ncc3Br)ccc2n1]

The title compound (15 mg, 26.3%) was prepared in a manner similar to that in Example 492 step 6 from (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide and 1-(3-(2,6-dioxopiperidin-3-yl)-2-oxo-2,3-dihydrobenzo[d]oxazol-7-yl)azetidine-3-carbaldehyde.

¹H NMR (500 MHz, DMSO) δ 11.80 (s, 1H), 11.20 (s, 1H), 8.56 (d, J=8.8 Hz, 1H), 8.27 (s, 1H), 8.21 (s, 1H), 8.03 (s, 1H), 7.88 (d, J=9.2 Hz, 1H), 7.44 (d, J=8.9 Hz, 1H), 7.32 (s, 1H), 7.01 (t, J=8.0 Hz, 1H), 6.76 (s, 1H), 6.58 (d, J=7.8 Hz, 1H), 6.25 (d, J=8.2 Hz, 1H), 5.30 (dd, J=12.9, 5.5 Hz, 1H), 4.11 (t, J=7.4 Hz, 2H), 3.76 (s, 3H), 3.65 (t, J=6.6 Hz, 2H), 3.28-3.23 (m, 2H), 2.93-2.90 (m, 6H), 2.68-2.64 (m, 4H), 2.60-2.51 (m, 5H), 2.41-2.37 (m, 3H), 2.31-2.27 (m, 3H), 2.15-2.11 (m, 1H), 1.98 (d, J=13.3 Hz, 6H), 1.84 (d, J=10.5 Hz, 2H), 1.58-1.52 (m, 2H), 1.32 (t, J=7.6 Hz, 3H), 0.77 (s, 3H). [M+H]⁺=1034.7

### Example 449: 3-(7-(3-((4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)methyl)azetidin-1-yl)-5-fluoro-2-oxobenzo[d]oxazol-3(2H)-yl)piperidine-2,6-dione

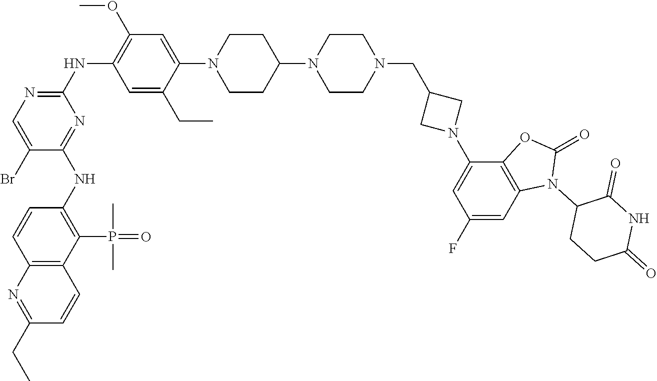 [SMILES: CCc1ccc2c(P(C)(C)=O)c(Nc3nc(Nc4cc(CC)c(N5CCC(N6CCN(CC7CN(c8cc(F)cc9c8oc(=O)n9C8CCC(=O)NC8=O)C7)CC6)CC5)cc4OC)ncc3Br)ccc2n1]

The title compound (20.25 mg, 41%) was prepared in a manner similar to that in Example 492 step 6 from (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide and 1-(3-(2,6-dioxopiperidin-3-yl)-5-fluoro-2-oxo-2,3-dihydrobenzo[d]oxazol-7-yl)azetidine-3-carbaldehyde.

¹H NMR (500 MHz, DMSO) δ 11.80 (s, 1H), 11.18 (s, 1H), 8.55 (d, J=9.0 Hz, 1H), 8.27 (s, 1H), 8.21 (s, 1H), 8.00 (s, 1H), 7.87 (d, J=9.0 Hz, 1H), 7.44 (d, J=8.9 Hz, 1H), 7.33 (s, 1H), 6.75 (s, 1H), 6.56 (dd, J=8.5, 2.3 Hz, 1H), 6.10 (dd, J=11.9, 2.3 Hz, 1H), 5.28 (dd, J=12.8, 5.2 Hz, 1H), 4.13 (t, J=7.6 Hz, 2H), 3.76 (s, 3H), 3.68 (t, J=6.7 Hz, 2H), 3.32 (s, 2H), 2.95-2.92 (m, 5H), 2.85-2.81 (m, 1H), 2.68-2.64 (m, 4H), 2.57-2.53 (m, 1H), 2.41-2.37 (m, 4H), 2.28 (s, 3H), 2.16-2.06 (m, 1H), 2.02 (s, 3H), 1.98 (s, 6H), 1.84 (d, J=11.0 Hz, 2H), 1.55-1.51 (m, 2H), 1.32 (t, J=7.6 Hz, 3H), 0.77 (s, 3H). [M+H]⁺=1052.6.

### Example 450: 3-(7-(4-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)piperidin-1-yl)-2-oxobenzo[d]oxazol-3(2H)-yl)piperidine-2,6-dione

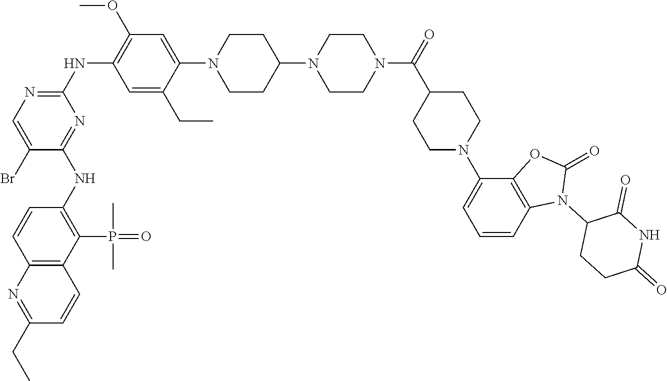 [SMILES: CCc1ccc2c(P(C)(C)=O)c(Nc3nc(Nc4cc(CC)c(N5CCC(N6CCN(C(=O)C7CCN(c8cccc9c8oc(=O)n9C8CCC(=O)NC8=O)CC7)CC6)CC5)cc4OC)ncc3Br)ccc2n1]

The title compound (50 mg, 55%) was prepared in a manner similar to that in Example 447.

¹H NMR (500 MHz, DMSO) δ 11.79 (s, 1H), 11.20 (s, 1H), 8.65-8.52 (m, 1H), 8.29 (s, 1H), 8.21 (s, 1H), 8.00 (s, 1H), 7.87 (s, 1H), 7.44 (d, J=8.9 Hz, 1H), 7.33 (s, 1H), 7.09 (t, J=8.2 Hz, 1H), 6.81-6.69 (m, 3H), 5.33 (d, J=18.5 Hz, 1H), 3.76 (s, 3H), 3.71 (d, J=11.9 Hz, 2H), 3.55 (s, 2H), 3.47 (s, 2H), 3.01-2.78 (m, 9H), 2.70-2.62 (m, 4H), 2.55 (d, J=10.0 Hz, 2H), 2.36 (d, J=1.9 Hz, 1H), 2.29 (d, J=7.8 Hz, 2H), 2.14 (t, J=5.2 Hz, 1H), 1.98 (d, J=13.3 Hz, 7H), 1.84 (s, 2H), 1.73 (s, 4H), 1.58 (d, J=11.9 Hz, 2H), 1.31 (d, J=7.6 Hz, 3H), 0.77 (s, 3H). [M+H]⁺=1076.3.

### Example 451: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-3-methyl-2-oxo-2,3-dihydro-1H-benzo[d]imidazol-1-yl)piperidine-2,6-dione

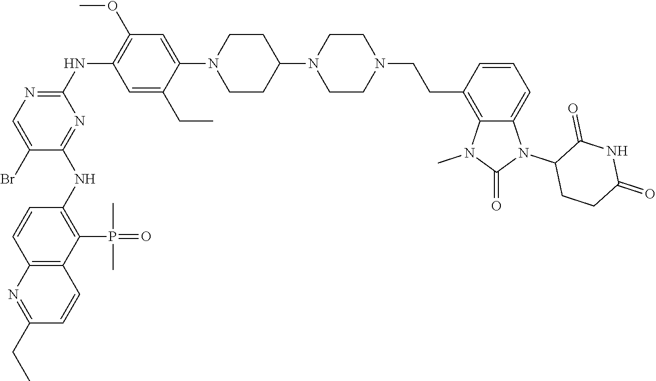 [SMILES: CCc1ccc2c(P(C)(C)=O)c(Nc3nc(Nc4cc(CC)c(N5CCC(N6CCN(CCc7cccc8c7n(C)c(=O)n8C7CCC(=O)NC7=O)CC6)CC5)cc4OC)ncc3Br)ccc2n1]

The title compound (50 mg, 55.2%) was prepared in a manner similar to that in Example 318. ¹H NMR (500 MHz, DMSO) δ 11.80 (s, 1H), 11.09 (s, 1H), 8.56 (d, J=8.9 Hz, 1H), 8.30 (s, J=8.9 Hz, 1H), 8.21 (s, 1H), 8.01 (s, 1H), 7.87 (d, J=9.3 Hz, 1H), 7.44 (d, J=8.9 Hz, 1H), 7.33 (s, 1H), 7.03-6.94 (m, 2H), 6.91 (dd, J=6.3, 2.8 Hz, 1H), 6.76 (s, 1H), 5.37 (dd, J=12.7, 5.4 Hz, 1H), 3.76 (s, 3H), 3.59 (s, 3H), 3.09-3.04 (m, 2H), 2.94 (t, J=9.7 Hz, 4H), 2.90 (d, J=7.0 Hz, 1H), 2.86 (d, J=5.5 Hz, 1H), 2.73 (dd, J=13.0, 4.5 Hz, 1H), 2.71-2.66 (m, 2H), 2.66-2.63 (m, 1H), 2.61 (d, J=3.6 Hz, 1H), 2.55 (t, J=8.6 Hz, 7H), 2.30 (s, 4H), 1.98 (d, J=13.3 Hz, 7H), 1.85 (d, J=12.0 Hz, 2H), 1.61-1.48 (m, 2H), 1.32 (t, J=7.6 Hz, 3H), 0.78 (s, 3H). [M+H]⁺=1006.2

### Example 452: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-5-fluoro-3-methyl-2-oxo-2,3-dihydro-1H-benzo[d]imidazol-1-yl)piperidine-2,6-dione

### Step 1: 7-bromo-6-fluoro-1-methyl-1,3-dihydro-2H-benzo[d]imidazol-2-one

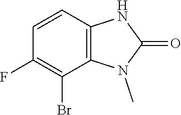

A mixture of 6-bromo-5-fluoro-N1-methylbenzene-1,2-diamine (2.8 g, 12.8 mmol) and CDI (2.5 g, 15.4 mmol) in THF (40 mL) was stirred in a flask at 70° C. for 4 hrs. The reaction was diluted with H₂O (160 mL), and the layers were separated. The aqueous layer was extracted with EtOAc (100 mL×3). The combined organic layers were washed with brine (100 mL×3), dried over Na₂SO₄, filtered and concentrated under vacuum. The residue was purified by column chromatography to afford 7-bromo-6-fluoro-1-methyl-1,3-dihydro-2H-benzo[d]imidazol-2-one (2.8 g, 89.3%). [M+H]⁺=245.0.

### Step 2: 3-(4-bromo-5-fluoro-3-methyl-2-oxo-2,3-dihydro-1H-benzo[d]imidazol-1-yl)-1-(4-methoxybenzyl)piperidine-2,6-dione

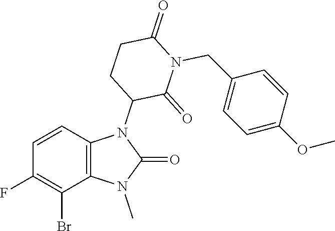 [SMILES: COc1ccc(CN2C(=O)CCC(n3c(=O)n(C)c4c(Br)c(F)ccc43)C2=O)cc1]

To a solution of 7-bromo-6-fluoro-1-methyl-1,3-dihydro-2H-benzo[d]imidazol-2-one (2.6 g, 10.7 mmol) was added sodium tert-butoxide (1 M in THF, 21.4 mmol, 21.4 mL) at 0° C. After stirring at 20° C. for 2 hrs, a solution of 1-(4-methoxybenzyl)-2,6-dioxopiperidin-3-yl trifluoromethanesulfonate (6.1 g, 16.0 mmol) in THF (50 mL) was added at 0° C. The mixture was stirred at 20° C. for another 2 hrs. The reaction was diluted with sat. aq. sodium bicarbonate (160 mL), and the layers were separated. The aqueous layer was extracted with DCM (200 mL×3). The combined organic layers were washed with brine (150 mL×3), dried over Na₂SO₄, filtered and concentrated under vacuum to afford 3-(4-bromo-5-fluoro-3-methyl-2-oxo-2,3-dihydro-1H-benzo[d]imidazol-1-yl)-1-(4-methoxybenzyl)piperidine-2,6-dione (2.8 g, 55%). [M+H]⁺=476.1.

### Step 3: 3-(4-bromo-5-fluoro-3-methyl-2-oxo-2,3-dihydro-1H-benzo[d]imidazol-1-yl)piperidine-2,6-dione

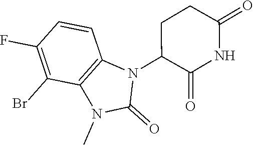 [SMILES: Cn1c(=O)n(C2CCC(=O)NC2=O)c2ccc(F)c(Br)c21]

A mixture of 3-(4-bromo-5-fluoro-3-methyl-2-oxo-2,3-dihydro-1H-benzo[d]imidazol-1-yl)-1-(4-methoxybenzyl)piperidine-2,6-dione (1.0 g, 2.1 mmol) and MsOH (2.0 g, 21 mmol) in toluene (10 mL) was stirred in a flask at 100° C. for 4 hrs. The reaction was diluted with H₂O (80 mL), and the layers were separated. The aqueous layer was extracted with DCM (60 mL×3). The combined organic layers were washed with brine (100 mL×3), dried over Na₂SO₄, filtered and concentrated under vacuum. The residue was purified by column chromatography to afford 3-(4-bromo-5-fluoro-3-methyl-2-oxo-2,3-dihydro-1H-benzo[d]imidazol-1-yl)piperidine-2,6-dione (670 mg, 90%). [M+H]⁺=356.0.

### Step 4: (E)-3-(4-(2-ethoxyvinyl)-5-fluoro-3-methyl-2-oxo-2,3-dihydro-1H-benzo[d]imidazol-1-yl)piperidine-2,6-dione

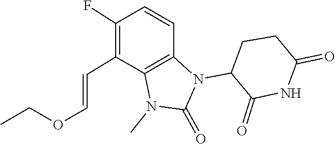 [SMILES: CCO/C=C/c1c(F)ccc2c1n(C)c(=O)n2C1CCC(=O)NC1=O]

A mixture of 3-(4-bromo-5-fluoro-3-methyl-2-oxo-2,3-dihydro-H-benzo[d]imidazol-1-yl)piperidine-2,6-dione (670 mg, 1.9 mmol), Pd(dtbpf)Cl₂ (130 mg, 0.2 mmol), (E)-2-(2-ethoxyvinyl)-4,4,5,5-tetramethyl-1,3,2-dioxaborolane (560 mg, 2.8 mmol), CsF (574 mg, 3.8 mmol), DMF (20 mL) and H₂O (3 mL) was stirred in a flask at 100° C. for 2 hrs under nitrogen atmosphere. The reaction was diluted with H₂O (160 mL) and EtOAc, and the layers were separated. The aqueous layer was extracted with DCM (100 mL×3). The combined organic layers were washed with brine (100 mL×3), dried over Na₂SO₄, filtered and concentrated under vacuum. The residue was purified by column chromatography to afford (E)-3-(4-(2-ethoxyvinyl)-5-fluoro-3-methyl-2-oxo-2,3-dihydro-TH-benzo[d]imidazol-1-yl)piperidine-2,6-dione (510 mg, 77.9%). [M+H]⁺=348.1.

### Step 5: 2-(1-(2,6-dioxopiperidin-3-yl)-5-fluoro-3-methyl-2-oxo-2,3-dihydro-1H-benzo[d]imidazol-4-yl)acetaldehyde

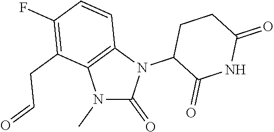 [SMILES: Cn1c(=O)n(C2CCC(=O)NC2=O)c2ccc(F)c(CC=O)c21]

(E)-3-(4-(2-ethoxyvinyl)-5-fluoro-3-methyl-2-oxo-2,3-dihydro-1H-benzo[d]imidazol-1-yl)piperidine-2,6-dione (510 mg, 1.5 mmol) was stirred in FA (40 mL) at RT for 2 hrs. The mixture was concentrated under vacuum to afford 2-(1-(2,6-dioxopiperidin-3-yl)-5-fluoro-3-methyl-2-oxo-2,3-dihydro-TH-benzo[d]imidazol-4-yl)acetaldehyde (460 mg, 97.9%), which was used without further purification. [M+H]⁺=320.1.

### Step 6: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-5-fluoro-3-methyl-2-oxo-2,3-dihydro-TH-benzo[d]imidazol-1-yl)piperidine-2,6-dione

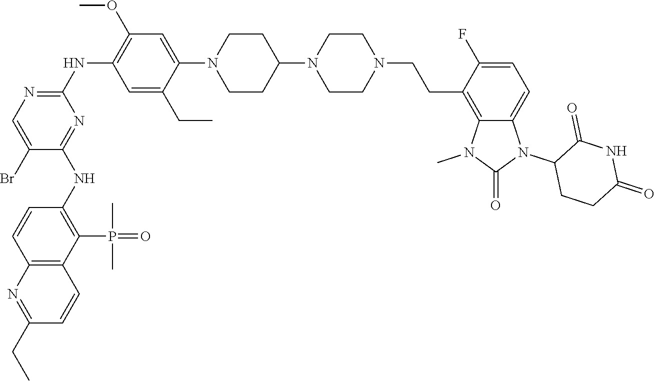 [SMILES: CCc1ccc2c(P(C)(C)=O)c(Nc3nc(Nc4cc(CC)c(N5CCC(N6CCN(CCc7c(F)ccc8c7n(C)c(=O)n8C7CCC(=O)NC7=O)CC6)CC5)cc4OC)ncc3Br)ccc2n1]

The title compound (14 mg, 26.2%) was prepared in a manner similar to that in Example 492 step 6 from (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide and 2-(1-(2,6-dioxopiperidin-3-yl)-5-fluoro-3-methyl-2-oxo-2,3-dihydro-1H-benzo[d]imidazol-4-yl)acetaldehyde.

¹H NMR (500 MHz, DMSO) δ 11.80 (s, 1H), 11.11 (s, 1H), 8.56 (d, J=8.8 Hz, 1H), 8.25 (m, 1H), 8.21 (s, 1H), 8.01 (s, 1H), 7.87 (d, J=9.3 Hz, 1H), 7.44 (d, J=8.9 Hz, 1H), 7.33 (s, 1H), 7.00 (dd, J=8.6, 4.3 Hz, 1H), 6.89 (dd, J=10.5, 8.7 Hz, 1H), 6.76 (s, 1H), 5.37 (dd, J=12.7, 5.4 Hz, 1H), 3.76 (s, 3H), 3.59 (s, 3H), 3.22 (m, 2H), 3.11-3.04 (m, 2H), 2.98-2.82 (m, 5H), 2.75-2.62 (m, 4H), 2.62-2.51 (m, 8H), 2.31-2.27 (m, 3H), 2.01-1.96 (m, 7H), 1.84 (d, J=10.5 Hz, 2H), 1.54 (dd, J=20.2, 11.4 Hz, 2H), 1.32 (t, J=7.6 Hz, 3H), 0.77 (s, 3H). [M+H]⁺=1024.7

### Example 453: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-3-ethyl-2-oxo-2,3-dihydro-TH-benzo[d]imidazol-1-yl)piperidine-2,6-dione

### Step 1: 2,6-bis(benzyloxy)-N-(3-bromo-2-nitrophenyl)pyridin-3-amine

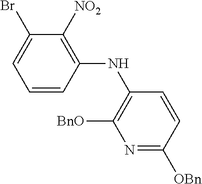 [SMILES: O=[N+]([O-])c1c(Br)cccc1Nc1ccc(OCc2ccccc2)nc1OCc1ccccc1]

To a flask was added of 2,6-bis(benzyloxy)pyridin-3-amine (8 g, 26.14 mmol), LiHMDS (1 M in THF, 31 mL, 31 mmol) and 1-bromo-3-fluoro-2-nitrobenzene(5.7 g, 26.14 mmol) in THF at −78° C. After stirring for 1 h at −78° C., the reaction mixture was warmed to room temperature and stirred for another 3 h under nitrogen atmosphere. The resulting mixture was concentrated under vacuum. Purification by silica gel column (PE:EA 1:1) afforded 2,6-bis(benzyloxy)-N-(3-bromo-2-nitrophenyl)pyridin-3-amine (8.1 g, 60.44%). [M+H]⁺=506.2.

### Step 2: NT-(2,6-bis(benzyloxy)pyridin-3-yl)-3-bromobenzene-1,2-diamine

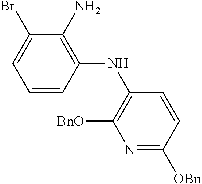 [SMILES: Nc1c(Br)cccc1Nc1ccc(OCc2ccccc2)nc1OCc1ccccc1]

A mixture of 2,6-bis(benzyloxy)-N-(3-bromo-2-nitrophenyl)pyridin-3-amine (8 g, 15.84 mmol), Fe (5.3 g, 94.64 mmol), NH4Cl (21.1 g, 39.60 mmol), MeOH (240 m1) and H₂O (120 m1) was stirred at 70° C. overnight under nitrogen atmosphere. The resulting mixture was filtered, and the solid was washed with EA. The filtrate was diluted with H₂O (300 mL) and extracted with EA (200 mL×3). The combined organic layers were washed with brine (200 mL×3), dried over anhydrous Na₂SO₄, filtered and concentrated under reduced pressure. Purification by silica gel column (DCM:MeOH 20:1) afforded N1-(2,6-bis(benzyloxy)pyridin-3-yl)-3-bromobenzene-1,2-diamine (5.9 g, 77.63%). [M+H]⁺=476.3.

### Step 3: 1-(2,6-bis(benzyloxy)pyridin-3-yl)-4-bromo-1,3-dihydro-2H-benzo[d]imidazol-2-one

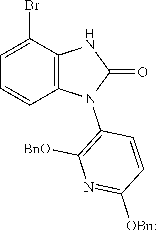

To a 250-mL round-bottom flask was added N1-(2,6-bis(benzyloxy)pyridin-3-yl)-3-bromobenzene-1,2-diamine (5.8 g, 12.21 mmol), ACN (60 mL), and CDI (3.6 g, 12.16 mmol). The resulting solution was stirred for 1.5 hr at 25° C. The resulting mixture was diluted with H₂O (150 mL) and extracted with EA (80 mL×3). The combined organic layers were washed with brine (100 mL×3), dried over anhydrous Na₂SO₄, filtered and concentrated under reduced pressure. Purification by silica gel column with (PE:EA 5:1) afforded 4.7 g (77.04%) of 1-(2,6-bis(benzyloxy)pyridin-3-yl)-4-bromo-1,3-dihydro-2H-benzo[d]imidazol-2-one, [M+H]⁺=502.3.

### Step 4: 1-(2,6-bis(benzyloxy)pyridin-3-yl)-4-bromo-3-ethyl-1,3-dihydro-2H-benzo[d]imidazol-2-one

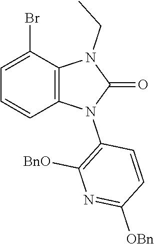 [SMILES: CCn1c(=O)n(-c2ccc(OCc3ccccc3)nc2OCc2ccccc2)c2cccc(Br)c21]

Into a 100-mL round-bottom flask were placed 1-(2,6-bis(benzyloxy)pyridin-3-yl)-4-bromo-1,3-dihydro-2H-benzo[d]imidazol-2-one (1.00 g, 1.99 mmol), DMF (10 mL), Cs₂CO₃ (1.9 g, 5.84 mmol), and EtI (479 mL, 5.98 mmol). The resulting solution was stirred overnight at room temperature. The residue was applied onto a silica gel column with petroleum ether/ethyl acetate (100:0˜ 70:30). The collected fractions were combined and concentrated under vacuum. This resulted in 1.00 g (94.70%) of 1-(2,6-bis(benzyloxy)pyridin-3-yl)-4-bromo-3-ethyl-1,3-dihydro-2H-benzo[d]imidazol-2-one, [M+H]⁺=530.1.

### Step 5: 4-(2-(benzyloxy)ethyl)-1-(2,6-bis(benzyloxy)pyridin-3-yl)-3-ethyl-1,3-dihydro-2H-benzo[d]imidazol-2-one

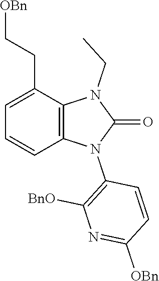 [SMILES: CCn1c(=O)n(-c2ccc(OCc3ccccc3)nc2OCc2ccccc2)c2cccc(CCOCc3ccccc3)c21]

To a solution of 1-(2,6-bis(benzyloxy)pyridin-3-yl)-4-bromo-3-ethyl-1,3-dihydro-2H-benzo[d]imidazol-2-one (1 g, 1.89 mmol), [(2-bromoethoxy)methyl]benzene (490 mg, 2.29 mmol) and benzamidine (60 mg, 0.38 mmol) in DMA (10 mL) were added Mn (310 mg, 5.64 mmol), nickel(II) iodide (120 mg, 0.38 mmol), NaI (140 mg, 0.93 mmol) and TFA (110 mg, 0.97 mmol) at 0° C. The mixture was stirred for 15 min at room temperature and then 2 h at 100° C. under nitrogen atmosphere. The reaction was quenched with H₂O (300 mL) at room temperature. The resulting mixture was filtered, and the solid was washed with EA (100 mL). The filtrate was extracted with EA (100 mL×3). The combined organic layers were washed with brine (100 mL×3), dried over anhydrous Na₂SO₄, filtered and concentrated under reduced pressure. The residue was applied onto a silica gel column with PE:EA (100:0-75:25). This resulted in 4-(2-(benzyloxy)ethyl)-1-(2,6-bis(benzyloxy)pyridin-3-yl)-3-ethyl-1,3-dihydro-2H-benzo[d]imidazol-2-one (110 mg, 10.4%), [M+H]⁺=586.3.

### Step 6: 3-(3-ethyl-4-(2-hydroxyethyl)-2-oxo-2,3-dihydro-1H-benzo[d]imidazol-1-yl)piperidine-2,6-dione

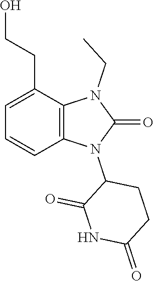 [SMILES: CCn1c(=O)n(C2CCC(=O)NC2=O)c2cccc(CCO)c21]

Into a 50-mL round-bottom flask were placed 4-(2-(benzyloxy)ethyl)-1-(2,6-bis(benzyloxy)pyridin-3-yl)-3-ethyl-1,3-dihydro-2H-benzo[d]imidazol-2-one (110 mg, 0.34 mmol), THF (4 mL), and Pd/C (110 mg, 10 wt. %, wet). The resulting solution was stirred overnight at 25° C. under hydrogen atmosphere. The resulting mixture was filtered and concentrated under vacuum, which afforded 42.5 mg (71.3%) of 3-(3-ethyl-4-(2-hydroxyethyl)-2-oxo-2,3-dihydro-1H-benzo[d]imidazol-1-yl)piperidine-2,6-dione, which was used without further purification. [M+H]⁺=318.2

¹H NMR (500 MHz, DMSO) δ 11.10 (d, J=3.2 Hz, 1H), 7.31-7.15 (m, 1H), 7.07-6.86 (m, 3H), 5.40-5.35 (m, 1H), 4.86 (t, J=5.2 Hz, 2H), 4.08-4.04 (m, 2H), 3.86-3.58 (m, 2H), 3.04-2.57 (m, 4H), 1.30-1.12 (m, 3H).

### Step 7: 2-(1-(2,6-dioxopiperidin-3-yl)-3-ethyl-2-oxo-2,3-dihydro-1H-benzo[d]imidazol-4-yl)acetaldehyde

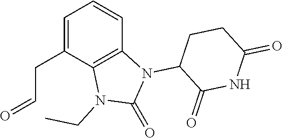 [SMILES: CCn1c(=O)n(C2CCC(=O)NC2=O)c2cccc(CC=O)c21]

The title compound (130 mg, 89.2%) was prepared in a manner similar to that in Example 486 step 8 from 3-(3-ethyl-4-(2-hydroxyethyl)-2-oxo-2,3-dihydro-1H-benzo[d]imidazol-1-yl)piperidine-2,6-dione and DMP. [M+H]⁺=316.1.

### Step 8: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-3-ethyl-2-oxo-2,3-dihydro-1H-benzo[d]imidazol-1-yl)piperidine-2,6-dione

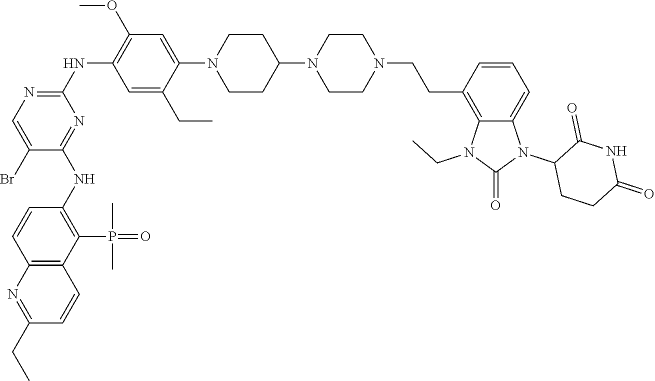 [SMILES: CCc1ccc2c(P(C)(C)=O)c(Nc3nc(Nc4cc(CC)c(N5CCC(N6CCN(CCc7cccc8c7n(CC)c(=O)n8C7CCC(=O)NC7=O)CC6)CC5)cc4OC)ncc3Br)ccc2n1]

The title compound (26 mg, 23.5%) was prepared in a manner similar to that in Example 492 step 6 from (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide and 2-(1-(2,6-dioxopiperidin-3-yl)-3-ethyl-2-oxo-2,3-dihydro-1H-benzo[d]imidazol-4-yl)acetaldehyde.

¹H NMR (500 MHz, DMSO) δ 11.80 (s, 1H), 11.11 (s, 1H), 8.56 (d, J=8.9 Hz, 1H), 8.27 (s, 1H), 8.21 (s, 1H), 8.03 (s, 1H), 7.88 (d, J=9.1 Hz, 1H), 7.44 (d, J=8.9 Hz, 1H), 7.32 (s, 1H), 6.98 (t, J=6.5 Hz, 2H), 6.96-6.90 (m, 1H), 6.76 (s, 1H), 5.37 (dd, J=12.5, 5.2 Hz, 1H), 4.01 (q, J=6.9 Hz, 2H), 3.76 (s, 3H), 3.28-3.22 (m, 2H), 2.94 (m, 7H), 2.77-2.52 (m, 12H), 2.32-2.26 (m, 3H), 2.05-2.01 (m, 1H), 1.99 (d, J=13.3 Hz, 6H), 1.85 (d, J=10.9 Hz, 2H), 1.58-1.52 (m, 2H), 1.32 (t, J=7.6 Hz, 3H), 1.24 (t, J=7.0 Hz, 3H), 0.77 (s, 3H). [M+H]⁺=1020.7

### Example 454: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-3-cyclopropyl-2-oxo-2,3-dihydro-1H-benzo[d]imidazol-1-yl)piperidine-2,6-dione

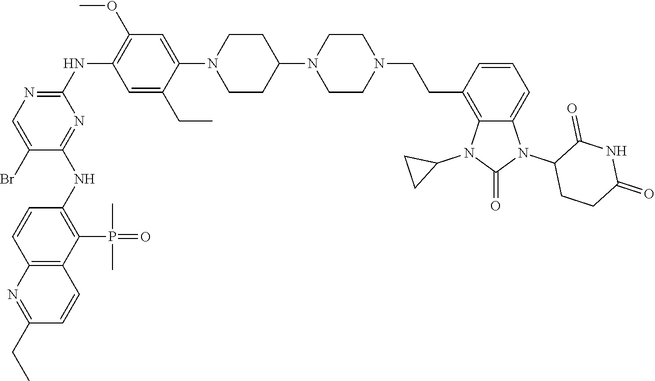 [SMILES: CCc1ccc2c(P(C)(C)=O)c(Nc3nc(Nc4cc(CC)c(N5CCC(N6CCN(CCc7cccc8c7n(C7CC7)c(=O)n8C7CCC(=O)NC7=O)CC6)CC5)cc4OC)ncc3Br)ccc2n1]

The title compound (33 mg, 28.9%) was prepared in a manner similar to that in Example 492.

¹H NMR (500 MHz, DMSO) δ 11.80 (s, 1H), 11.10 (s, 1H), 8.56 (d, J=8.9 Hz, 1H), 8.27 (s, 1H), 8.21 (s, 1H), 8.03 (s, 1H), 7.88 (d, J=9.3 Hz, 1H), 7.44 (d, J=8.9 Hz, 1H), 7.32 (s, 1H), 7.02-6.96 (m, 1H), 6.94 (d, J=6.7 Hz, 2H), 6.76 (s, 1H), 5.31 (dd, J=12.4, 5.2 Hz, 1H), 3.76 (s, 3H), 3.32-3.21 (m, 6H), 3.17-3.12 (m, 1H), 2.94-2.88 (m, 5H), 2.71-2.53 (m, 10H), 2.33-2.27 (m, 3H), 1.98 (d, J=13.3 Hz, 6H), 1.96-1.92 (m, 1H), 1.85 (d, J=10.7 Hz, 2H), 1.58-1.53 (m, 2H), 1.32 (t, J=7.6 Hz, 3H), 1.15-1.02 (m, 4H), 0.77 (s, 3H). [M+H]⁺=1032.7

### Example 455: 3-(7-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-y)piperazin-1-yl)ethyl)-2-oxobenzo[d]oxazol-3(2H)-yl)piperidine-2,6-dione

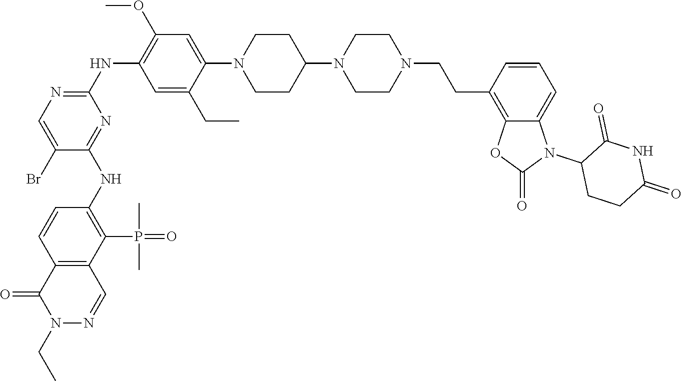 [SMILES: CCc1cc(Nc2ncc(Br)c(Nc3ccc4c(=O)n(CC)ncc4c3P(C)(C)=O)n2)c(OC)cc1N1CCC(N2CCN(CCc3cccc4c3oc(=O)n4C3CCC(=O)NC3=O)CC2)CC1]

The title compound (15 mg, 15%) was prepared in a manner similar to that in Example 492.

¹H NMR (500 MHz, DMSO) δ 12.11 (s, 1H), 11.21 (s, 1H), 8.60-8.53 (m, 2H), 8.32-7.98 (m, 3H), 7.33 (s, 1H), 7.18-6.98 (m, 3H), 6.72 (s, 1H), 5.36 (dd, J=12.9, 5.3 Hz, 1H), 4.16 (q, J=7.2 Hz, 2H), 4.00 (q, J=6.9 Hz, 2H), 3.00-2.91 (m, 2H), 2.90-2.81 (m, 3H), 2.74-2.60 (m, 5H), 2.58-2.54 (m, 9H), 2.40-2.36 (m, 2H), 2.29 (dd, J=12.0, 5.1 Hz, 1H), 2.19-2.10 (m, 1H), 2.08-1.93 (m, 6H), 1.88-1.76 (m, 2H), 1.65-1.51 (m, 2H), 1.31 (t, J=7.1 Hz, 3H), 1.25 (t, J=6.9 Hz, 3H), 0.92-0.77 (m, 3H); [M+H]⁺=1024.6.

### Example 456: 3-(6-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)propyl)-2-oxobenzo[dl oxazol-3(2H)-yl)piperidine-2,6-dione

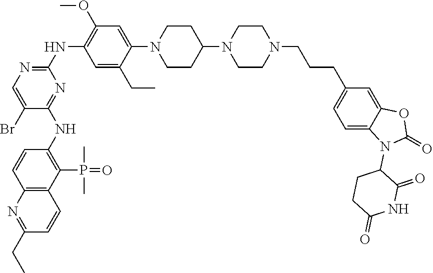 [SMILES: CCc1ccc2c(P(C)(C)=O)c(Nc3nc(Nc4cc(CC)c(N5CCC(N6CCN(CCCc7ccc8c(c7)oc(=O)n8C7CCC(=O)NC7=O)CC6)CC5)cc4OC)ncc3Br)ccc2n1]

The title compound (22.98 mg, 43.7%) was prepared in a manner similar to that in Example 492.

¹H NMR (500 MHz, DMSO) δ 11.80 (s, 1H), 11.20 (s, 1H), 8.55 (d, J=9.0 Hz, 1H), 8.27 (s, 1H), 8.17 (s, 1H), 8.00 (s, 1H), 7.87 (d, J=9.2 Hz, 1H), 7.44 (d, J=8.9 Hz, 1H), 7.32 (s, 1H), 7.27 (s, 1H), 7.16 (d, J=8.1 Hz, 1H), 7.05 (d, J=7.2 Hz, 1H), 6.75 (s, 1H), 5.35 (dd, J=12.9, 5.3 Hz, 1H), 3.75 (s, 3H), 2.93 (t, J=7.5 Hz, 6H), 2.75-2.58 (m, 8H), 2.56-2.52 (m, 2H), 2.36 (s, 3H), 2.27 (d, J=7.2 Hz, 5H), 2.18-2.08 (m, 1H), 2.00 (s, 3H), 1.98 (s, 3H), 1.84 (d, J=11.0 Hz, 2H), 1.77-1.64 (m, 2H), 1.63-1.43 (m, 2H), 1.32 (t, J=7.6 Hz, 3H), 0.77 (s, 3H). [M+H]⁺=1007.5.

### Example 457: 3-(6-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-5-fluoro-2-oxobenzo[d]oxazol-3(2H)-yl)piperidine-2,6-dione

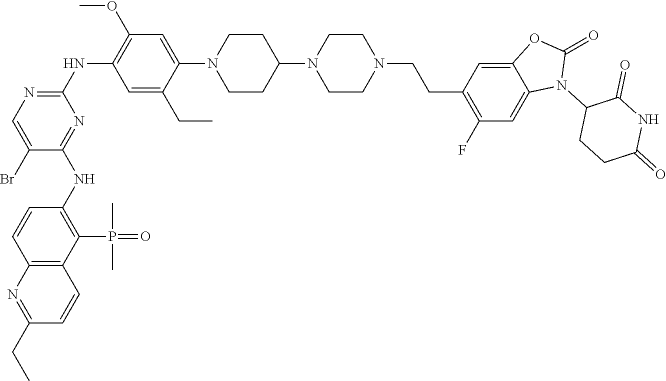 [SMILES: CCc1ccc2c(P(C)(C)=O)c(Nc3nc(Nc4cc(CC)c(N5CCC(N6CCN(CCc7cc8oc(=O)n(C9CCC(=O)NC9=O)c8cc7F)CC6)CC5)cc4OC)ncc3Br)ccc2n1]

The title compound (24.65 mg, 31%) was prepared in a manner similar to that in Example 492.

¹H NMR (500 MHz, DMSO) δ 11.80 (s, 1H), 11.20 (s, 1H), 8.55 (d, J=8.9 Hz, 1H), 8.27 (s, 1H), 8.18 (s, 1H), 8.00 (s, 1H), 7.87 (d, J=9.2 Hz, 1H), 7.42 (dd, J=14.1, 7.5 Hz, 2H), 7.36-7.21 (m, 2H), 6.75 (s, 1H), 5.34 (d, J=13.1 Hz, 1H), 3.76 (s, 3H), 2.94-2.92 (m, 6H), 2.78-2.76 (m, 3H), 2.72-2.60 (m, 4H), 2.53-2.51 (m, 4H), 2.49-2.46 (m, 3H), 2.29 (d, J=4.3 Hz, 4H), 2.15-2.12 (m, 1H), 1.99 (s, 3H), 1.97 (s, 3H), 1.84 (d, J=10.8 Hz, 2H), 1.55-1.53 (m, 2H), 1.33-1.31 (m, 3H), 0.77 (s, 3H). [M+H]⁺=1011.6.

### Example 458: 3-(6-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-7-fluoro-2-oxobenzo[d]oxazol-3(2H)-yl)piperidine-2,6-dione

### Step 1: 2-amino-6-fluorophenol

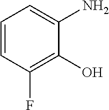

To a 100-mL round-bottom flask was added 2-fluoro-6-nitrophenol (10 g, 63.69 mmol), THF (100 mL), and Pd/C (10 g, 10 wt. %, wet). The mixture was stirred overnight at 25° C. under hydrogen atmosphere. The resulting mixture was then filtered and concentrated under vacuum to afford 7 g (86.53%) of 2-amino-6-fluorophenol, which was used without further purification. [M+H]⁺=128.4.

### Step 2: 7-fluorobenzo[d]oxazol-2(3H)-one

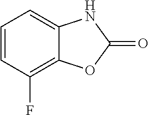 [SMILES: O=c1[nH]c2cccc(F)c2o1]

Into a 500-mL round-bottom flask were placed 2-amino-6-fluorophenol (7 g, 55.11 mmol), ACN (210 mL), and CDI (11 g, 67.90 mmol). The resulting solution was stirred for 1.5 hr at 25° C. The resulting mixture was diluted with H₂O (400 mL) and extracted with EA (150 mL×3). The combined organic layers were washed with brine (200 mL×3) and concentrated under reduced pressure. The residue was applied onto a silica gel column with PE:EA (5:1) This resulted in 5 g (59.3%) of 7-fluorobenzo[d]oxazol-2(3H)-one, [M+H]⁺=154.2.

### Step 3: 6-bromo-7-fluorobenzo[d]oxazol-2(3H)-one

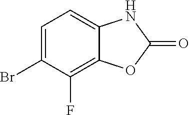 [SMILES: O=c1[nH]c2ccc(Br)c(F)c2o1]

Into a 100-mL round-bottom flask were placed 7-fluorobenzo[d]oxazol-2(3H)-one (5 g, 32.67 mmol), DMF (40 mL), and NBS(6.3 g, 35.94 mmol). The reaction was stirred overnight at room temperature. The resulting mixture was diluted with H₂O (500 mL) and extracted with EA (150 mL×3). The combined organic layers were washed with brine (200 mL×3), dried over anhydrous sodium sulfate, filtered and concentrated under vacuum. The residue was applied onto a silica gel column with PE/EA (5:1). This resulted in 4 g (54.7%) of 6-bromo-7-fluorobenzo[d]oxazol-2(3H)-one, [M+H]⁺=232.1.

### Step 4: 3-(2,6-bis(benzyloxy)pyridin-3-yl)-6-bromo-7-fluorobenzo[d]oxazol-2(3H)-one

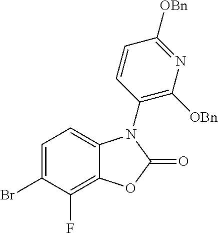 [SMILES: O=c1oc2c(F)c(Br)ccc2n1-c1ccc(OCc2ccccc2)nc1OCc1ccccc1]

A mixture of 6-bromo-7-fluorobenzo[d]oxazol-2(3H)-one (4 g, 17.31 mmol), 2,6-bis(benzyloxy)-3-(4,4,5,5-tetramethyl-1,3,2-dioxaborolan-2-yl)pyridine (7.3 g, 17.60 mmol), Cu(OAc)₂ (3.1 g, 17.30 mmol), pyridine (4.1 g, 51.90 mmol) and 4A MS (2.5 g) in dioxane (40 mL) was stirred at 80° C. overnight under oxygen atmosphere. The resulting mixture was concentrated under vacuum. The residue was applied onto a silica gel column with PE:EA (1:1). This resulted in 3-(2,6-bis(benzyloxy)pyridin-3-yl)-6-bromo-7-fluorobenzo[d]oxazol-2(3H)-one (4 g, 44.44%), [M+H]⁺=521.2.

### Step 5: ethyl 2-(3-(2,6-bis(benzyloxy)pyridin-3-yl)-7-fluoro-2-oxo-2,3-dihydrobenzo[d]oxazol-6-yl)acetate

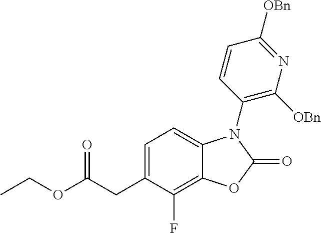 [SMILES: CCOC(=O)Cc1ccc2c(oc(=O)n2-c2ccc(OCc3ccccc3)nc2OCc2ccccc2)c1F]

Into a 30-mL microwave vial #1, purged and maintained with nitrogen atmosphere, was placed Zn (1.1 g, 16.82 mmol), THF (8 mL), and TMSCI (86 μl, 73.1 mmol). The resulting solution was stirred for 10 min at room temperature. To this was added ethyl bromoacetate (1124 mg, 6.73 mmol). The resulting solution was stirred for 1.5 hrs at 60° C. Into a 100-mL 3-necked round-bottom flask, purged and maintained with nitrogen atmosphere, was placed 3-(2,6-bis(benzyloxy)pyridin-3-yl)-6-bromo-7-fluorobenzo[d]oxazol-2(3H)-one (1 g, 1.923 mmol), THF (10 mL), XPhos (184 mg, 3.85 mmol), and Pd₂(dba)₃ (176 mg, 0.192 mmol). To the above mixture was added the solution in vial #1 by a syringe through a millipore filter. The resulting solution was stirred for another 3 hrs at 60° C. The resulting solution was diluted with EA (50 mL). The pH value of the solution was adjusted to <7 with 2N HCl, and the layers were separated. The aqueous layer was extracted with ethyl acetate (50 mL×3). The combined organic layers were dried over anhydrous sodium sulfate, filtered and concentrated under vacuum. The residue was applied onto a silica gel column with EA/PE (1:1). This resulted in 600 mg (59.1%) of ethyl 2-(3-(2,6-bis(benzyloxy)pyridin-3-yl)-7-fluoro-2-oxo-2,3-dihydrobenzo[d]oxazol-6-yl)acetate, [M+H]⁺=529.3.

### Step 6: 6-((2,6-bis(benzyloxy)pyridin-3-yl)amino)-2-fluoro-3-(2-hydroxyethyl)phenol

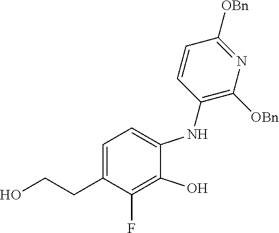 [SMILES: OCCc1ccc(Nc2ccc(OCc3ccccc3)nc2OCc2ccccc2)c(O)c1F]

Into a 50-mL round-bottom flask were placed ethyl 2-(3-(2,6-bis(benzyloxy)pyridin-3-yl)-7-fluoro-2-oxo-2,3-dihydrobenzo[d]oxazol-6-yl)acetate (500 mg, 0.946 mmol), and THF (5 mL). LiBH₄ (103 mg, 4.7 mmol) was then added at room temperature. The reaction was stirred overnight at room temperature. The reaction was then quenched by the addition of H₂O (60 mL). The resulting mixture was extracted with EA (20 mL×3). The combined organic layers were washed with brine (40 mL×3), dried over Na₂SO₄, filtered and concentrated under reduced pressure. Purification by silica gel column with DCM/MeOH (4:1) resulted in 290 mg (66.5%) of 6-((2,6-bis(benzyloxy)pyridin-3-yl)amino)-2-fluoro-3-(2-hydroxyethyl)phenol, [M+H]⁺=461.4.

### Step 7: 6-((2,6-bis(benzyloxy)pyridin-3-yl)amino)-3-(2-((tert-butyldimethylsilyl)oxy)ethyl)-2-fluorophenol

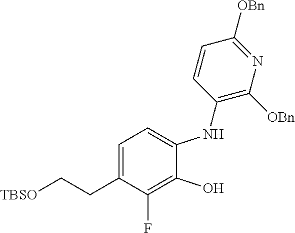 [SMILES: CC(C)(C)[Si](C)(C)OCCc1ccc(Nc2ccc(OCc3ccccc3)nc2OCc2ccccc2)c(O)c1F]

Into a 100-mL round-bottom flask were placed 6-((2,6-bis(benzyloxy)pyridin-3-yl)amino)-2-fluoro-3-(2-hydroxyethyl)phenol (270 mg, 0.58 mmol), DCM (5 mL), TBSCI (97 mg, 0.61 mml), and imidazole (64 mg, 0.94 mmol). The resulting solution was stirred for 1 hr at 25° C. The resulting mixture was diluted with H₂O (30 mL), and the layers were separated. The aqueous layer was extracted with EA (20 mL×3). The combined organic layers were washed with brine (50 mL×3), dried over Na₂SO₄, filtered and concentrated under reduced pressure. Purification by silica gel column with DCM/MeOH (10:1) resulted in 230 mg (68.2%) of 6-((2,6-bis(benzyloxy)pyridin-3-yl)amino)-3-(2-((tert-butyldimethylsilyl)oxy)ethyl)-2-fluorophenol. [M+H]⁺=575.2.

### Step 8: 3-(2,6-bis(benzyloxy)pyridin-3-yl)-6-(2-((tert-butyldimethylsilyl)oxy)ethyl)-7-fluorobenzo[d]oxazol-2(3H)-one

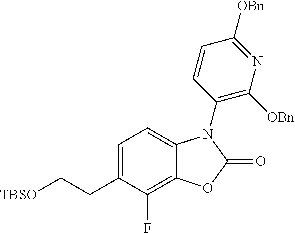 [SMILES: CC(C)(C)[Si](C)(C)OCCc1ccc2c(oc(=O)n2-c2ccc(OCc3ccccc3)nc2OCc2ccccc2)c1F]

Into a 25-mL round-bottom flask were placed 6-((2,6-bis(benzyloxy)pyridin-3-yl)amino)-3-(2-((tert-butyldimethylsilyl)oxy)ethyl)-2-fluorophenol (220 mg, 0.38 mmol), ACN (5 mL), and CDI (81 mg, 0.50 mmol). The resulting solution was stirred for 1.5 hr at 25° C. The resulting mixture was diluted with H₂O (30 mL), and the layers were separated. The aqueous layer was extracted with EA (20 mL×3). The combined organic layers were washed with brine (50 mL×3), dried over Na₂SO₄, filtered and concentrated under reduced pressure. The residue was applied onto a silica gel column with PE:EA (5:1). This resulted in 170 mg (74.2%) of 3-(2,6-bis(benzyloxy)pyridin-3-yl)-6-(2-((tert-butyldimethylsilyl)oxy)ethyl)-7-fluorobenzo[d]oxazol-2(3H)-one, [M+H]⁺=601.4.

### Step 9: 3-(6-(2-((tert-butyldimethylsilyl)oxy)ethyl)-7-fluoro-2-oxobenzo[d]oxazol-3(2H)-yl)piperidine-2,6-dione

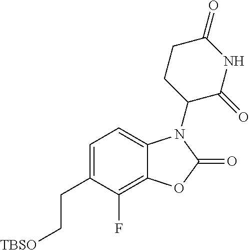 [SMILES: CC(C)(C)[Si](C)(C)OCCc1ccc2c(oc(=O)n2C2CCC(=O)NC2=O)c1F]

Into a 25-mL round-bottom flask was placed 3-(2,6-bis(benzyloxy)pyridin-3-yl)-6-(2-((tert-butyldimethylsilyl)oxy)ethyl)-7-fluorobenzo[d]oxazol-2(3H)-one (170 mg, 0.28 mmol), THF (3 mL), and Pd/C (170 mg, 10 wt. %, wet). The resulting solution was stirred overnight at 25° C. under hydrogen atmosphere. The resulting mixture was filtered and concentrated under vacuum. This resulted in 110 mg (92.4%) of 3-(6-(2-((tert-butyldimethylsilyl)oxy)ethyl)-7-fluoro-2-oxobenzo[d]oxazol-3(2H)-yl)piperidine-2,6-dione, which was used without further purification ([M+H]⁺=423.2).

### Step 10: 3-(7-fluoro-6-(2-hydroxyethyl)-2-oxobenzo[d]oxazol-3(2H)-yl)piperidine-2,6-dione

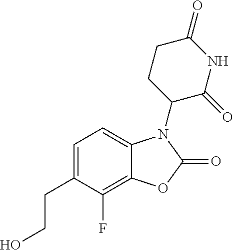 [SMILES: O=C1CCC(n2c(=O)oc3c(F)c(CCO)ccc32)C(=O)N1]

Into a 25-ml round-bottom flask were placed 3-(6-(2-((tert-butyldimethylsilyl)oxy)ethyl)-7-fluoro-2-oxobenzo[d]oxazol-3(2H)-yl)piperidine-2,6-dione (110 mg, 0.26 mmol), DCM (4 mL), and TFA (2 mL). The resulting solution was stirred for 2 h at 25° C. The resulting mixture was concentrated under vacuum. This resulted in 79.4 mg (99%) of 3-(7-fluoro-6-(2-hydroxyethyl)-2-oxobenzo[d]oxazol-3(2H)-yl)piperidine-2,6-dione, [M+H]⁺=308.2.

¹H NMR (500 MHz, DMSO) δ 11.24 (s, 1H), 7.21-7.02 (m, 2H), 5.42-5.34 (m, 1H), 4.58-4.44 (m, 1H), 3.77-3.46 (m, 2H), 2.95-2.78 (m, 2H), 2.71-2.62 (m, 2H), 2.22-2.11 (m, 1H), 1.25 (d, J=4.0 Hz, 1H).

### Step 11: 2-(3-(2,6-dioxopiperidin-3-yl)-7-fluoro-2-oxo-2,3-dihydrobenzo[d]oxazol-6-yl)acetaldehyde

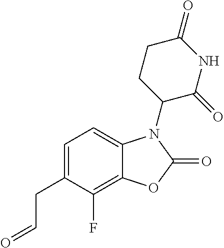 [SMILES: O=CCc1ccc2c(oc(=O)n2C2CCC(=O)NC2=O)c1F]

The title compound (60 mg, 80%) was prepared in a manner similar to that in Example 448 step 3 from 3-(7-fluoro-6-(2-hydroxyethyl)-2-oxobenzo[d]oxazol-3(2H)-yl)piperidine-2,6-dione and DMP.

### Step 12: 3-(6-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-7-fluoro-2-oxobenzo[d]oxazol-3(2H)-yl)piperidine-2,6-dione

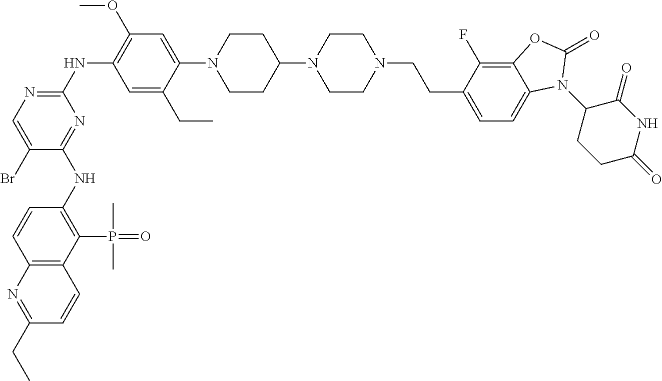 [SMILES: CCc1ccc2c(P(C)(C)=O)c(Nc3nc(Nc4cc(CC)c(N5CCC(N6CCN(CCc7ccc8c(oc(=O)n8C8CCC(=O)NC8=O)c7F)CC6)CC5)cc4OC)ncc3Br)ccc2n1]

The title compound (30 mg, 40.5%) was prepared in a manner similar to that in Example 492 step 6 from (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide and 2-(3-(2,6-dioxopiperidin-3-yl)-7-fluoro-2-oxo-2,3-dihydrobenzo[d]oxazol-6-yl)acetaldehyde.

¹H NMR (500 MHz, DMSO) δ 11.79 (s, 1H), 11.23 (s, 1H), 8.56 (d, J=8.9 Hz, 1H), 8.29 (d, J=17.1 Hz, 1H), 8.21 (s, 1H), 8.00 (s, 1H), 7.87 (d, J=9.3 Hz, 1H), 7.44 (d, J=8.9 Hz, 1H), 7.33 (s, 1H), 7.21-7.15 (m, 1H), 7.06 (d, J=8.2 Hz, 1H), 6.75 (s, 1H), 5.38 (dd, J=13.0, 5.3 Hz, 1H), 3.76 (s, 3H), 2.93-2.88 (m, 9H), 2.67 (dd, J=14.2, 8.4 Hz, 5H), 2.49-2.38 (m, 5H), 2.28 (s, 4H), 2.22-2.14 (m, 1H), 2.02-1.95 (m, 7H), 1.82 (s, 2H), 1.53 (dd, J=20.1, 11.4 Hz, 2H), 1.32 (t, J=7.6 Hz, 3H), 0.77 (s, 3H). [M+H]⁺=1011.3.

### Example 459: (R)-3-(4-(2-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)ethyl)-2,6-difluorophenyl)piperidine-2,6-dione Step 1:1-bromo-2-chloro-4-cyclopropoxy-5-nitrobenzene

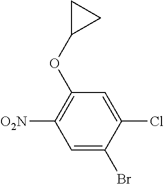 [SMILES: O=[N+]([O-])c1cc(Br)c(Cl)cc1OC1CC1]

To a solution of 1-bromo-2-chloro-4-fluoro-5-nitrobenzene (4 g, 15.7 mmol) in DMSO (50 mL) was added cyclopropanol (912 mg, 15.7 mmol) and K₂CO₃ (4.34 g, 31.4 mmol) at 20° C. Then the mixture was warmed to 70° C. and stirred for 16 hrs. Then the mixture was diluted with EA (200 mL), washed with water (100 mL×2) and brine (100 mL×2). Then the organic layer was dried over anhydrous sodium sulfate, filtered and concentrated in vacuo. The residue was purified by Combi-Flash (silica column, 80 g, PE:EA=10:1) to give the product (3.5 g, 76.2%).

### Step 2: tert-butyl 4-(1-(2-bromo-5-cyclopropoxy-4-nitrophenyl)piperidin-4-yl)piperazine-1-carboxylate

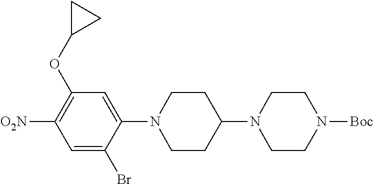 [SMILES: CC(C)(C)OC(=O)N1CCN(C2CCN(c3cc(OC4CC4)c([N+](=O)[O-])cc3Br)CC2)CC1]

To a solution of 1-bromo-2-chloro-4-cyclopropoxy-5-nitrobenzene (3.5 g, 12.0 mmol) in MeCN (50 mL) was added tert-butyl 4-(piperidin-4-yl)piperazine-1-carboxylate (3.56 g, 13.2 mmol) and K₂CO₃ (3.31 g, 24.0 mmol) at 25° C. Then the mixture was stirred at 80° C. for 16 hrs.

Then the mixture was cooled to rt and filtered. The solid was washed with EA. Then the filtrate was concentrated in vacuo. The residue was purified by Combi-Flash (silica column, 80 g, DCM: MeOH=30:1) to give tert-butyl 4-(1-(2-bromo-5-cyclopropoxy-4-nitrophenyl)piperidin-4-yl)piperazine-1-carboxylate (4 g, 63.3%). [M+H]⁺=525.3.

### Step 3: tert-butyl 4-(1-(5-cyclopropoxy-4-nitro-2-vinylphenyl)piperidin-4-yl)piperazine-1-carboxylate

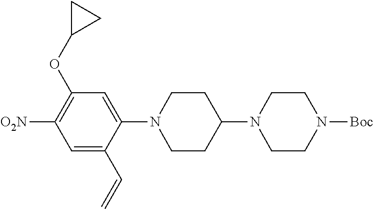 [SMILES: C=Cc1cc([N+](=O)[O-])c(OC2CC2)cc1N1CCC(N2CCN(C(=O)OC(C)(C)C)CC2)CC1]

To a suspension of tert-butyl 4-(1-(2-bromo-5-cyclopropoxy-4-nitrophenyl)piperidin-4-yl)piperazine-1-carboxylate (2 g, 3.8 mmol) and 4,4,5,5-tetramethyl-2-vinyl-1,3,2-dioxaborolane (879 mg, 5.7 mmol) in dioxane (16 mL) and water (4 mL) was added K₂CO₃ (1.57 g, 11.4 mmol) and Pd(dppf)Cl₂ (139 mg, 0.19 mmol). The mixture was warmed to 100° C. and stirred for 16 hrs under nitrogen atmosphere. Then the mixture was cooled to rt and filtered. The filtrate was concentrated in vacuo. The residue was purified by Combi-Flash (silica column, 40 g, DCM: MeOH=15: 1) to give tert-butyl 4-(1-(5-cyclopropoxy-4-nitro-2-vinylphenyl)piperidin-4-yl)piperazine-1-carboxylate (1.4 g, 77.9%). [M+H]⁺=473.3.

### Step 4: tert-butyl 4-(1-(4-amino-5-cyclopropoxy-2-ethylphenyl)piperidin-4-yl)piperazine-1-carboxylate

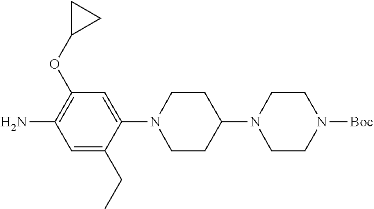 [SMILES: CCc1cc(N)c(OC2CC2)cc1N1CCC(N2CCN(C(=O)OC(C)(C)C)CC2)CC1]

To a suspension of tert-butyl 4-(1-(5-cyclopropoxy-4-nitro-2-vinylphenyl)piperidin-4-yl)piperazine-1-carboxylate (1.4 g, 3.0 mmol) in MeOH (20 mL) was added Pd/C (1 g, 10 wt. %, wet). The mixture was stirred at rt for 16 hrs under hydrogen atmosphere. Then the mixture was filtered and the solid was washed with MeOH. The filtrate was concentrated in vacuo to afford tert-butyl 4-(1-(4-amino-5-cyclopropoxy-2-ethylphenyl)piperidin-4-yl)piperazine-1-carboxylate (1.2 g, 90.0%). [M+H]⁺=445.3.

### Step 5: (6-((5-bromo-2-((2-cyclopropoxy-5-ethyl-4-(4-(piperazin-1-yl)piperidin-1-yl)phenyl)amino)pyrimidin-4-yl)amino)-2-methylquinolin-5-yl)dimethylphosphine oxide

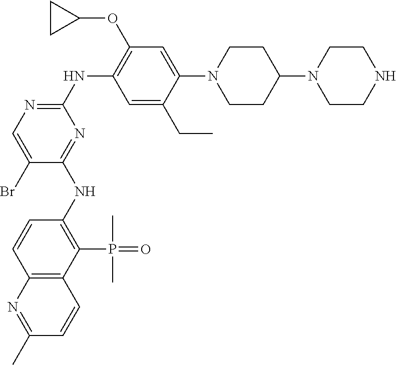 [SMILES: CCc1cc(Nc2ncc(Br)c(Nc3ccc4nc(C)ccc4c3P(C)(C)=O)n2)c(OC2CC2)cc1N1CCC(N2CCNCC2)CC1]

To a solution of (6-((5-bromo-2-chloropyrimidin-4-yl)amino)-2-methylquinolin-5-yl)dimethylphosphine oxide (553 mg, 1.3 mmol) in n-BuOH (10 mL) was added tert-butyl 4-(1-(4-amino-5-cyclopropoxy-2-ethylphenyl)piperidin-4-yl)piperazine-1-carboxylate (600 mg, 1.3 mmol) at 20° C. 4-Methylbenzenesulfonic acid (783 mg, 4.6 mmol) was added to the reaction mixture at 20° C. Then the mixture was stirred at 100° C. for 13 hrs. The mixture was diluted with water (100 mL), adjusted to pH=8 with 5N NaOH solution and then extracted with DCM (150 mL×3). The combined organic layers were washed with brine (150 mL×3), dried over Na₂SO₄, filtered and concentrated in vacuum. The residue was purified by column chromatography (DCM/MeOH (0.5% NH₄OH)=10/1 to 5/1). (6-((5-bromo-2-((2-cyclopropoxy-5-ethyl-4-(4-(piperazin-1-yl)piperidin-1-yl)phenyl)amino)pyrimidin-4-yl)amino)-2-methylquinolin-5-yl)dimethylphosphine oxide (500 mg, 52%) was obtained. [M+H]⁺=733.2.

### Step 6: (R)-3-(4-(2-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)ethyl)-2,6-difluorophenyl)piperidine-2,6-dione

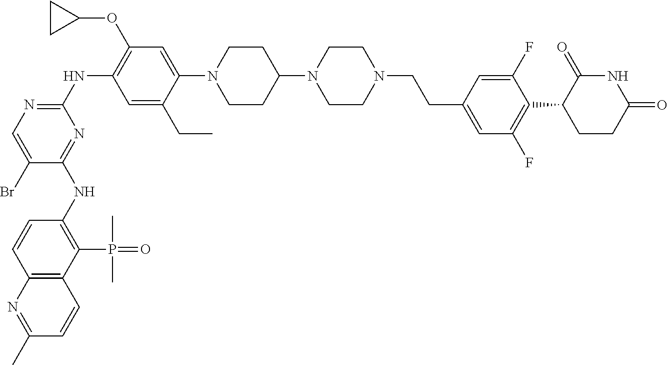 [SMILES: CCc1cc(Nc2ncc(Br)c(Nc3ccc4nc(C)ccc4c3P(C)(C)=O)n2)c(OC2CC2)cc1N1CCC(N2CCN(CCc3cc(F)c([C@H]4CCC(=O)NC4=O)c(F)c3)CC2)CC1]

The title compound (32 mg, 48%) was prepared in a manner similar to that in Example 484 step 15 from (6-((5-bromo-2-((2-cyclopropoxy-5-ethyl-4-(4-(piperazin-1-yl)piperidin-1-yl)phenyl)amino)pyrimidin-4-yl)amino)-2-methylquinolin-5-yl)dimethylphosphine oxide and (R)-2-(4-(2,6-dioxopiperidin-3-yl)-3,5-difluorophenyl)acetaldehyde. ¹H NMR (500 MHz, DMSO) δ 11.74 (s, 1H), 10.95 (s, 1H), 8.58 (d, J=8.5 Hz, 1H), 8.27 (d, J=7.5 Hz, 1H), 8.21 (s, 1H), 7.85-7.84 (m, 2H), 7.43 (d, J=8.5 Hz, 1H), 7.39 (s, 1H), 7.04 (d, J=10.0 Hz, 2H), 6.98 (s, 1H), 4.20 (dd, J=12.5, 5.0 Hz, 1H), 3.81 (dq, J=9.0, 3.0 Hz, 1H), 2.98 (d, J=10.5 Hz, 2H), 2.85-2.76 (m, 4H), 2.75-2.51 (m, 14H), 2.19-2.15 (m, 5H), 1.99-1.95 (m, 9H), 1.69-1.53 (m, 2H), 0.75 (t, J=7.5, 3H), 0.74-0.69 (m, 2H), 0.61-0.56 (m, 2H). [M+H]⁺=984.3.

### Example 460: (R)-3-(4-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

### Step 1: 2-ethyl-6-nitroquinazoline

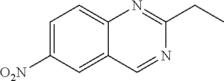

To a solution of 2-fluoro-5-nitrobenzaldehyde (10.0 g, 59.17 mmol) in MeCN (150 mL) was added propionimidamide hydrochloride (9.59 g, 88.75 mmol) and K₂CO₃ (20.4 g, 147.93 mmol) at room temperature. The resulting mixture was stirred at 80° C. overnight. The reaction was concentrated to give the crude residue, which was purified by silica gel column chromatography (DCM: MeOH=100: 0-98: 2 gradient elution) to give a mixture (3.6 g, 30%) containing the desired product. [M+H]⁺=204.2.

### Step 2: 2-ethylquinazolin-6-amine

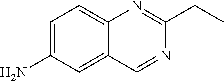

To a solution of 2-ethyl-6-nitroquinazoline (3.6 g, 17.73 mmol) in THF (100 mL)/H₂O (20 mL) was added Fe (4.96 g, 88.67 mmol) and NH₄Cl (4.7 g, 88.67 mmol) at 25° C. Then the mixture was stirred at 25° C. overnight. The mixture was filtered through a pad of Celite and washed with EA (150 mL) and H₂O (60 mL). The filtrate was separated and the organic layer was concentrated to give the crude residue, which was purified by silica gel column chromatography (DCM: MeOH=100: 0-20: 1 gradient elution) to give a mixture (1.8 g, 58%) containing the desired product. [M+H]+=174.2.

### Step 3: 2-ethyl-5-iodoquinazolin-6-amine

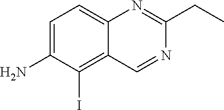

To a solution of 2-ethylquinazolin-6-amine (1.8 g, 10.4 mmol) in AcOH (30 mL) was added ICI (15.6 mL, 15.6 mmol) at 20° C. Then the mixture was stirred at 20° C. for 3 hours. Then the mixture was adjusted to pH=8 with sat. aq. NaHCO₃ and extracted with DCM (2×100 mL). The organic phase was washed with brine (80 mL), dried over Na₂SO₄, filtered and concentrated in vacuum to afford the desired product (2.6 g, 84%). [M+H]+=300.1.

### Step 4: (6-amino-2-ethylquinazolin-5-yl)dimethylphosphine oxide

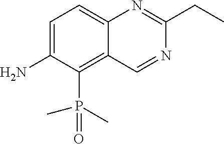 [SMILES: CCc1ncc2c(P(C)(C)=O)c(N)ccc2n1]

To a solution of 2-ethyl-5-iodoquinazolin-6-amine (2.6 g, 8.69 mmol) and dimethylphosphine oxide (1.36 g, 17.39 mmol) in dioxane (100 mL) was added K₃PO₄ (4.6 g, 21.73 mmol) at 20° C. Pd(OAc)₂ (390 mg, 1.74 mmol) and Xantphos (1.0 g, 1.74 mmol) were added to the mixture at 20° C. The suspension was degassed under vacuum and purged with N2 three times. Then the mixture was stirred at 100° C. overnight. The mixture was filtered and concentrated in vacuum. The residue was purified by silica gel column chromatography (DCM: MeOH=100: 0-10: 1 gradient elution) to give the desired product (2.1 g, 97%). [M+H]+=250.1.

### Step 5: (6-((5-bromo-2-chloropyrimidin-4-yl)amino)-2-ethylquinazolin-5-yl)dimethylphosphine oxide

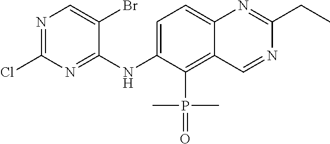 [SMILES: CCc1ncc2c(P(C)(C)=O)c(Nc3nc(Cl)ncc3Br)ccc2n1]

To a solution of (6-amino-2-ethylquinazolin-5-yl)dimethylphosphine oxide (2.1 g, 8.4 mmol) and 5-bromo-2,4-dichloropyrimidine (5.7 g, 25.2 mmol) in n-BuOH (90 mL) was added DIEA(3.3 g, 25.2 mmol) at room temperature. The resulting mixture was stirred at 120° C. overnight. The reaction was concentrated to afford the crude residue, which was purified by silica gel column chromatography (DCM: MeOH=100: 0-15: 1 gradient elution) to give the desired product (2.3 g, 62%).

¹H NMR (500 MHz, DMSO) δ 12.44 (s, 1H), 9.83 (s, 1H), 8.59 (s, 1H), 8.56 (dd, J=9.3, 3.8 Hz, 1H), 8.12 (d, J=9.3 Hz, 1H), 3.07 (q, J=7.6 Hz, 2H), 2.12-2.08 (m, 6H), 1.38 (t, J=7.6 Hz, 3H). [M+H]⁺=440.1.

### Step 6: 4-ethoxy-1-ethyl-2-fluorobenzene

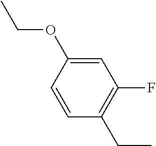

To a solution of 4-ethyl-3-fluorophenol (35 g, 0.25 mol) in DMF (200 mL) was added K₂CO₃ (69 g, 0.5 mol), and EtI (50.7 g, 0.32 mol). The mixture was stirred at 20-30° C. for 18 hours. The reaction was quenched by H₂O (200 mL) and extracted with EA (150 mL×2). The combined organic phases were washed with brine (300 mL×3), dried over anhydrous Na₂SO₄, filtered and concentrated in vacuum. The residue was purified by column chromatography with pure PE to give product. (35 g, 83.3%). [M+H]⁺=168.2.

### Step 7: 1-ethoxy-4-ethyl-5-fluoro-2-nitrobenzene

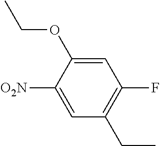

To a solution of 4-ethoxy-1-ethyl-2-fluorobenzene (35 g, 0.2 mol) in Ac₂O (100 mL) was added HNO₃ (23.4 g, 0.26 mol) dropwise at 0° C. The mixture was stirred at r.t. for 2hs. The reaction was then quenched with Na₂CO₃ solution (500 mL). The layers were separated and the organic layer was concentrated to afford the product (25 g, 58.7%) which was used in the next step without further purification. ¹H NMR (500 MHz, DMSO) δ 7.90 (d, J=8.0 Hz, 1H), 7.26 (d, J=12.0 Hz, 1H), 4.2 (q, J=7.0 Hz, 2H), 2.60 (q, J=7.5 Hz, 2H), 1.33 (t, J=7.0 Hz, 3H), 1.15 (t, J=7.5 Hz, 3H).

### Step 8: tert-butyl 4-(1-(5-ethoxy-2-ethyl-4-nitrophenyl)piperidin-4-yl)piperazine-1-carboxylate

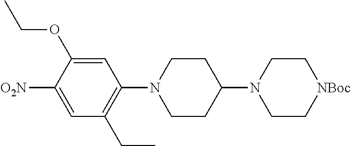 [SMILES: CCOc1cc(N2CCC(N3CCN(C(=O)OC(C)(C)C)CC3)CC2)c(CC)cc1[N+](=O)[O-]]

To a solution of 1-ethoxy-4-ethyl-5-fluoro-2-nitrobenzene (20 g, 94 mmol) in DMF (300 mL) was added tert-butyl 4-(piperidin-4-yl)piperazine-1-carboxylate (30 g, 112 mmol), and K₂CO₃ (32 g, 235 mmol). The mixture was stirred at 120° C. for 28 hours. The mixture was poured into ice water. The product (20 g, 46.1%) was isolated by filtration, which was used in the next step without further purification. ¹H NMR (500 MHz, DMSO) δ 7.74 (s, 1H), 6.73 (s, 1H), 4.19 (q, J=7.0 Hz, 2H), 3.30 (m, 4H), 3.23 (d, J=11.0 Hz, 2H), 2.71 (t, J=11.5 Hz, 2H), 2.57 (q, J=7.5 Hz, 2H), 2.47 (br s, 4H), 2.39 (t, J=11.0 Hz, 1H), 1.84 (d, J=11.5 Hz, 2H), 1.58 (q, J=10.5 Hz, 2H), 1.39 (s, 9H), 1.34 (t, J=7.5 Hz, 3H), 1.19 (t, J=7.5 Hz, 3H).

### Step 9: tert-butyl 4-(1-(4-amino-5-ethoxy-2-ethylphenyl)piperidin-4-yl)piperazine-1-carboxylate

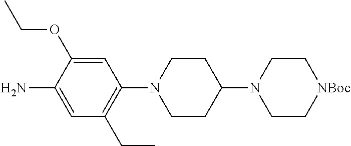 [SMILES: CCOc1cc(N2CCC(N3CCN(C(=O)OC(C)(C)C)CC3)CC2)c(CC)cc1N]

To a solution of tert-butyl 4-(1-(5-ethoxy-2-ethyl-4-nitrophenyl)piperidin-4-yl)piperazine-1-carboxylate (20 g, 94 mmol) in THF (150 mL) was added Pd/C (2 g, 10 wt. %, wet). The mixture was stirred at r.t. under hydrogen atmosphere (1 atm) for 48 h. The solid was filtered off. The filtrate was concentrated for next step directly without further purification. [M+H]⁺=433.4.

### Step 10: (6-((5-bromo-2-((2-ethoxy-5-ethyl-4-(4-(piperazin-1-yl)piperidin-1-yl)phenyl)amino)pyrimidin-4-yl)amino)-2-ethylquinazolin-5-yl)dimethylphosphine oxide

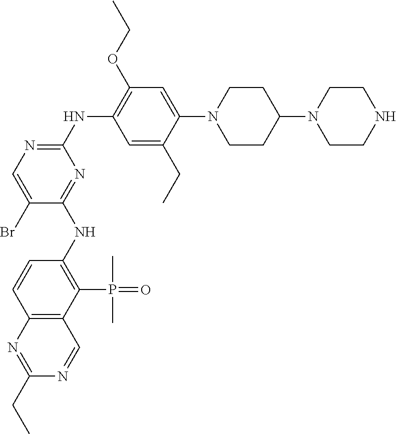 [SMILES: CCOc1cc(N2CCC(N3CCNCC3)CC2)c(CC)cc1Nc1ncc(Br)c(Nc2ccc3nc(CC)ncc3c2P(C)(C)=O)n1]

To a solution of (6-((5-bromo-2-chloropyrimidin-4-yl)amino)-2-ethylquinazolin-5-yl)dimethylphosphine oxide (1.0 g, 2.27 mmol) and tert-butyl 4-(1-(4-amino-5-ethoxy-2-ethylphenyl)piperidin-4-yl)piperazine-1-carboxylate (1.18 g, 2.73 mmol) in n-BuOH (30 mL) was added Ts-OH (1.17 g, 6.81 mmol) at room temperature. The resulting mixture was stirred at 100° C. overnight. The reaction was concentrated and basified with 0.5N NaOH (40 mL), then extracted with DCM (3×80 mL). The combined organic layers were dried over anhydrous Na₂SO₄, filtered and evaporated in vacuum to afford the crude residue, which was purified by silica gel column chromatography (DCM: MeOH=100: 0-5: 1 gradient elution) to give the desired product (720 mg, 43%). [M+H]+=736.2.

### Step 11: (R)-3-(4-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-1)ethyl)-2,6-difluorophenyl)piperidine-2,6-dione

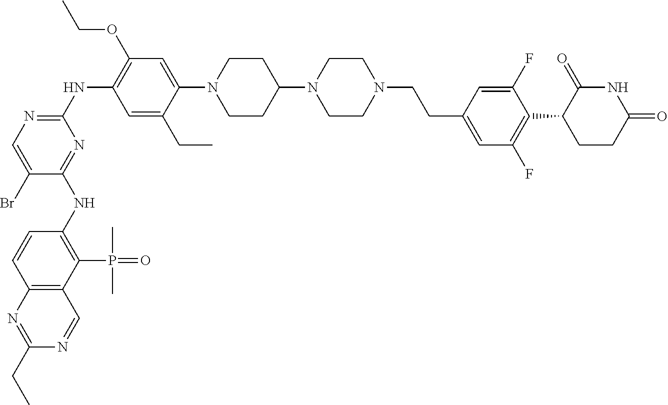 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)ncc3c2P(C)(C)=O)n1]

To a solution of (6-((5-bromo-2-((2-ethoxy-5-ethyl-4-(4-(piperazin-1-yl)piperidin-1-yl)phenyl)amino)pyrimidin-4-yl)amino)-2-ethylquinazolin-5-yl)dimethylphosphine oxide (60 mg, 0.082 mmol) and (R)-2-(4-(2,6-dioxopiperidin-3-yl)-3,5-difluorophenyl)acetaldehyde (33 mg, 0.123 mmol) in DCM (5 mL) was added sodium triacetoxyborohydride (60 mg, 0.28 mmol) at room temperature. The resulting mixture was stirred at room temperature for 1 hour. The reaction was quenched with water (10 mL) and extracted with DCM (2×10 mL). The combined organic layers were dried over anhydrous Na₂SO₄, filtered and evaporated in vacuum to afford the crude residue, which was purified by silica gel column chromatography (DCM: MeOH=100: 0-10: 1 gradient elution) to give an impure product, which was further purified with prep-HPLC to give the desired product (13.54 mg, 16%).

¹H NMR (500 MHz, DMSO) δ 11.52 (s, 1H), 10.95 (s, 1H), 9.91 (s, 1H), 8.42 (s, 1H), 8.25 (d, J=19.9 Hz, 1H), 7.94 (s, 1H), 7.85 (d, J=9.3 Hz, 1H), 7.33 (s, 1H), 7.03 (d, J=10.2 Hz, 2H), 6.70 (s, 1H), 4.20 (dd, J=12.9, 5.2 Hz, 1H), 3.99 (q, J=6.9 Hz, 2H), 3.05 (q, J=7.5 Hz, 2H), 2.91 (d, J=10.3 Hz, 2H), 2.81-2.74 (m, 3H), 2.65-2.57 (m, 3H), 2.54-2.49 (m, 7H), 2.46 (s, 3H), 2.27-2.21 (m, 3H), 2.13 (d, J=9.6 Hz, 1H), 2.02 (d, J=13.4 Hz, 7H), 1.82 (d, J=10.9 Hz, 2H), 1.52 (d, J=9.1 Hz, 2H), 1.38 (t, J=7.6 Hz, 3H), 1.25 (t, J=6.9 Hz, 3H), 0.68 (s, 3H); [M+H]⁺=987.7.

### Example 461: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3,5-dimethyl-2-oxo-2,3-dihydro-1H-benzo[d]imidazol-1-yl)piperidine-2,6-dione

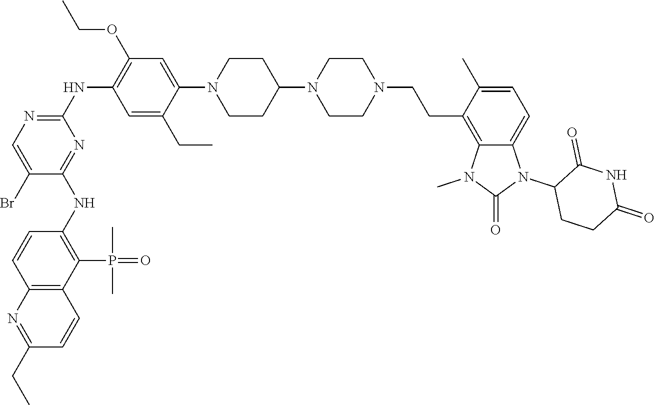 [SMILES: CCOc1cc(N2CCC(N3CCN(CCc4c(C)ccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound (32 mg, 45%) was prepared in a manner similar to that in Example 318. ¹H NMR (500 MHz, DMSO) δ 11.66 (s, 1H), 11.01 (s, 1H), 8.53 (d, J=8.6 Hz, 1H), 8.15 (s, 2H), 7.88-7.77 (m, 2H), 7.37 (s, 1H), 7.32 (s, 1H), 6.81 (s, 2H), 6.65 (s, 1H), 5.27 (dd, J=12.6, 5.3 Hz, 1H), 3.94 (d, J=7.0 Hz, 2H), 3.54 (s, 3H), 3.03-2.95 (m, 2H), 2.92-2.77 (m, 6H), 2.57-2.48 (m, 12H), 2.25 (s, 7H), 2.02-1.88 (m, 7H), 1.77 (d, J=10.6 Hz, 2H), 1.47 (dd, J=20.1, 11.0 Hz, 2H), 1.26 (t, J=7.6 Hz, 3H), 1.19 (t, J=6.9 Hz, 3H), 0.64 (s, 3H). [M+H]⁺=1034.4

### Example 462: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-6-fluoro-3-methyl-2-oxo-2,3-dihydro-1H-benzo[d]imidazol-1-yl)piperidine-2,6-dione

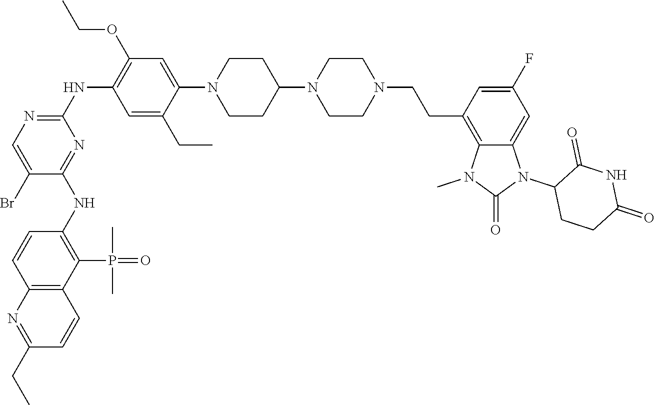 [SMILES: CCOc1cc(N2CCC(N3CCN(CCc4cc(F)cc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound (46 mg, 43%) was prepared in a manner similar to that in Example 318. ¹H NMR (500 MHz, DMSO) δ 11.73 (s, 1H), 11.10 (s, 1H), 8.60 (d, J=9.1 Hz, 1H), 8.27 (s, 1H), 8.22 (s, 1H), 8.01-7.82 (m, 2H), 7.45 (d, J=8.9 Hz, 1H), 7.38 (s, 1H), 7.01 (d, J=7.2 Hz, 1H), 6.80 (dd, J=11.0, 2.3 Hz, 1H), 6.71 (s, 1H), 5.35 (dd, J=12.4, 5.3 Hz, 1H), 4.00 (d, J=7.0 Hz, 2H), 3.57 (s, 3H), 3.11-3.03 (m, 2H), 2.93 (d, J=7.5 Hz, 4H), 2.84 (d, J=12.4 Hz, 1H), 2.73 (dt, J=17.1, 10.7 Hz, 2H), 2.62-2.55 (m, 12H), 2.26 (s, 3H), 2.02-1.92 (m, 7H), 1.83 (d, J=10.2 Hz, 2H), 1.61-1.41 (m, 2H), 1.32 (t, J=7.6 Hz, 3H), 1.25 (t, J=6.9 Hz, 3H), 0.71 (s, 3H). [M+H]⁺=1038.4

### Example 463: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

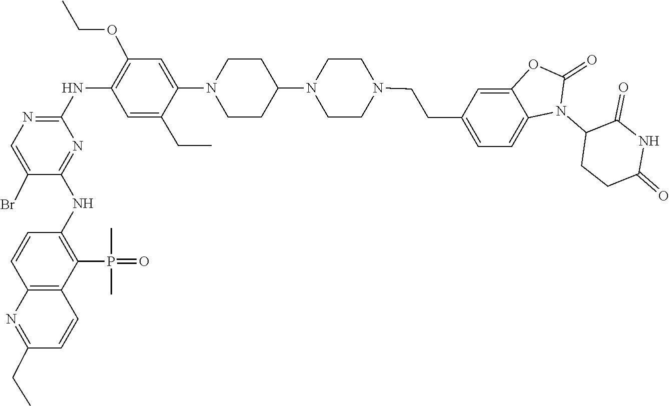 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2ccc3nc(CC)ccc3c2P(C)(C)=O)n1]

A mixture of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (the compound was obtained similar to example 484) (75 mg, 0.10 mmol) and 2-(3-(2,6-dioxopiperidin-3-yl)-2-oxo-2,3-dihydrobenzo[d]oxazol-6-yl)acetaldehyde (35 mg, 0.12 mmol) in DCM (8 mL) was stirred in a flask at room temperature for 10 min. To the mixture was added sodium triacetoxyborohydride (65 mg, 0.31 mmol), and the reaction was stirred at room temperature for another 0.5 h. Then the mixture was concentrated under vacuum to afford the crude product, which was purified with prep-HPLC chromatography (0.1% FA in water: acetonitrile=90:10˜ 50:50 gradient elution) to give the product (34 mg, 33.2%).

¹H NMR (500 MHz, DMSO) δ 11.73 (s, 1H), 11.20 (s, 1H), 8.60 (d, J=8.8 Hz, 1H), 8.24 (s, 1H), 8.22 (s, 1H), 7.91 (s, 1H), 7.87 (d, J=9.3 Hz, 1H), 7.45 (d, J=8.9 Hz, 1H), 7.38 (s, 1H), 7.30 (s, 1H), 7.16 (d, J=8.0 Hz, 1H), 7.07 (d, J=8.0 Hz, 1H), 6.70 (s, 1H), 5.35 (dd, J=12.9, 5.2 Hz, 1H), 4.00 (q, J=6.9 Hz, 2H), 3.32-3.19 (m, 6H), 2.99-2.86 (m, 5H), 2.80-2.57 (m, 7H), 2.48-2.39 (m, 3H), 2.29-2.24 (m, 3H), 2.16 (dd, J=10.7, 5.2 Hz, 1H), 1.98 (d, J=13.3 Hz, 6H), 1.82 (d, J=11.9 Hz, 2H), 1.52 (dd, J=19.9, 11.3 Hz, 2H), 1.32 (t, J=7.6 Hz, 3H), 1.25 (t, J=6.9 Hz, 3H), 0.71 (s, 3H). [M+H]⁺=1007.7

### Example 465: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-methyl-2-oxobenzo[d]oxazol-3(2H)-yl)piperidine-2,6-dione

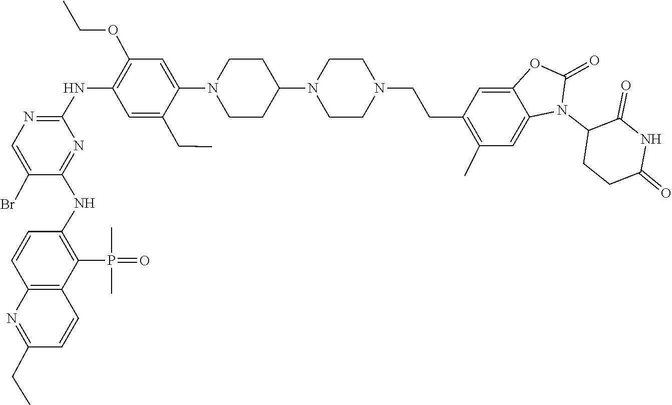 [SMILES: CCOc1cc(N2CCC(N3CCN(CCc4cc5oc(=O)n(C6CCC(=O)NC6=O)c5cc4C)CC3)CC2)c(CC)cc1Nc1ncc(Br)c(Nc2ccc3nc(CC)ccc3c2P(C)(C)=O)n1]

The title compound (42 mg, 36.8%) was prepared in a manner similar to that in Example 492. ¹H NMR (500 MHz, DMSO) δ 11.73 (s, 1H), 11.18 (s, 1H), 8.60 (d, J=8.9 Hz, 1H), 8.22 (s, 2H), 7.96-7.83 (m, 2H), 7.45 (d, J=8.9 Hz, 1H), 7.38 (s, 1H), 7.24-7.15 (m, 1H), 7.08 (s, 1H), 6.71 (s, 1H), 5.32 (dd, J=13.0, 5.3 Hz, 1H), 4.00 (q, J=7.0 Hz, 2H), 3.01-2.81 (m, 5H), 2.79-2.68 (m, 3H), 2.64 (dd, J=19.2, 8.3 Hz, 4H), 2.55 (d, J=7.8 Hz, 3H), 2.46 (d, J=7.8 Hz, 4H), 2.38-2.34 (m, 1H), 2.30 (s, 6H), 2.17-2.07 (m, 1H), 1.98 (d, J=13.3 Hz, 6H), 1.84 (d, J=11.3 Hz, 2H), 1.54 (dd, J=20.4, 11.2 Hz, 3H), 1.32 (s, 3H), 1.25 (s, 3H), 0.71 (s, 3H). [M+H]⁺=1021.4

### Example 466: 3-(4-(3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazine-1-carbonyl)azetidin-1-yl)-6-fluoro-3-methyl-2-oxo-2,3-dihydro-1H-benzo[d]imidazol-1-yl)piperidine-2,6-dione

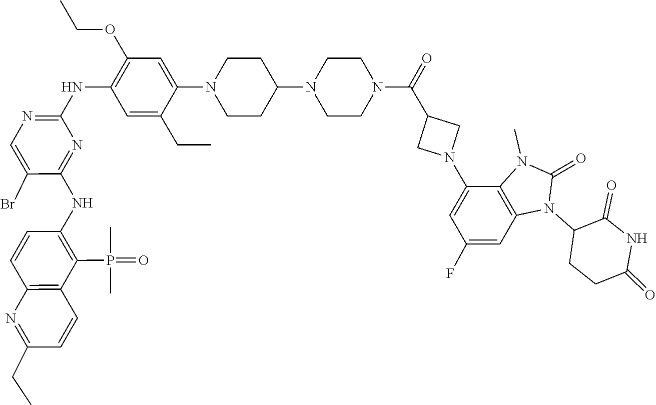 [SMILES: CCOc1cc(N2CCC(N3CCN(C(=O)C4CN(c5cc(F)cc6c5n(C)c(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound (45 mg, 54.3%) was prepared in a manner similar to that in Example 447. ¹H NMR (500 MHz, DMSO) δ 11.72 (s, 1H), 11.09 (s, 1H), 8.60 (d, J=8.9 Hz, 1H), 8.22 (s, 2H), 7.99-7.81 (m, 2H), 7.46 (t, J=10.0 Hz, 1H), 7.39 (s, 1H), 6.82-6.67 (m, 2H), 6.61-6.46 (m, 1H), 5.31 (dd, J=12.6, 5.1 Hz, 1H), 4.07 (t, J=6.4 Hz, 2H), 4.04-3.93 (m, 4H), 3.82-3.72 (m, 1H), 3.54 (s, 3H), 3.47-3.43 (m, 2H), 2.93 (q, J=7.4 Hz, 4H), 2.89-2.80 (m, 1H), 2.78-2.68 (m, 1H), 2.63 (t, J=16.1 Hz, 4H), 2.51 (s, 4H), 2.36 (s, 1H), 2.30-2.19 (m, 4H), 1.98 (d, J=13.3 Hz, 7H), 1.81 (d, J=10.7 Hz, 2H), 1.55 (dd, J=20.2, 11.2 Hz, 2H), 1.32 (s, 3H), 1.26 (s, 3H), 0.89-0.55 (m, 3H). [M+H]⁺=1093.3.

### Example 467: 3-(4-(3-((4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)methyl)azetidin-1-yl)-6-fluoro-3-methyl-2-oxo-2,3-dihydro-1H-benzo[d]imidazol-1-yl)piperidine-2,6-dione

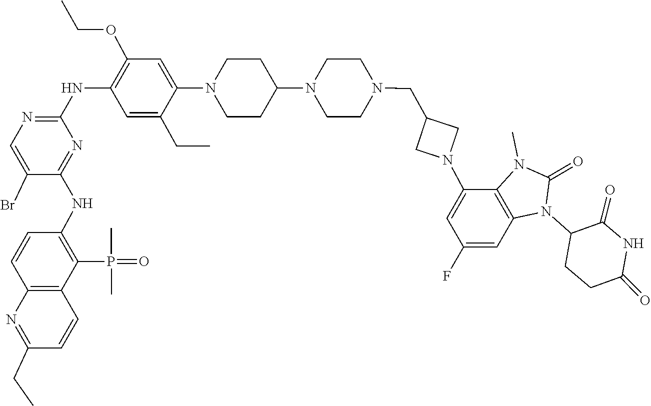 [SMILES: CCOc1cc(N2CCC(N3CCN(CC4CN(c5cc(F)cc6c5n(C)c(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound (25 mg, 35%) was prepared in a manner similar to that in Example 492. ¹H NMR (500 MHz, DMSO) δ 11.72 (s, 1H), 11.08 (s, 1H), 8.60 (d, J=8.4 Hz, 1H), 8.22 (s, 2H), 7.99-7.72 (m, 2H), 7.53-7.31 (m, 2H), 6.71 (s, 2H), 6.45 (dd, J=12.3, 2.0 Hz, 1H), 5.30 (dd, J=12.6, 5.1 Hz, 1H), 3.99 (s, 4H), 3.52 (s, 6H), 2.93 (d, J=7.4 Hz, 6H), 2.56 (d, J=7.1 Hz, 9H), 2.38 (d, J=20.0 Hz, 4H), 2.25 (d, J=7.1 Hz, 3H), 1.98 (d, J=13.3 Hz, 7H), 1.82 (d, J=11.9 Hz, 2H), 1.52 (d, J=9.0 Hz, 2H), 1.32 (t, J=7.6 Hz, 3H), 1.25 (s, 3H), 0.70 (s, 3H). [M+H]⁺=1079.4

### Example 468: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3-cyclopropyl-2-oxo-2,3-dihydro-1H-benzo[d]imidazol-1-yl)piperidine-2,6-dione

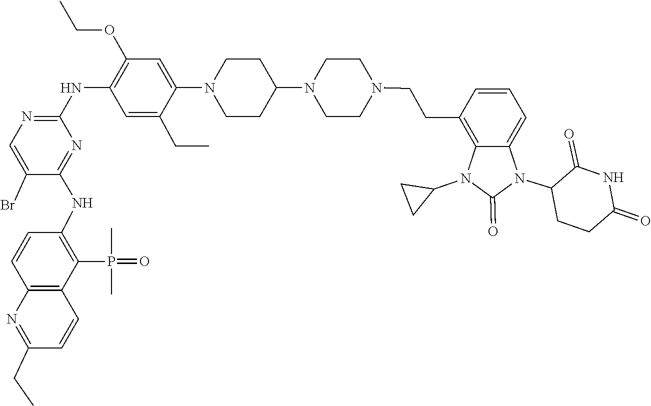 [SMILES: CCOc1cc(N2CCC(N3CCN(CCc4cccc5c4n(C4CC4)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound (25 mg, 27.3%) was prepared in a manner similar to that in Example 318. ¹H NMR (500 MHz, DMSO) δ 11.73 (s, 1H), 11.09 (s, 1H), 8.60 (d, J=8.8 Hz, 1H), 8.25 (s, 1H), 8.22 (s, 1H), 7.93 (s, 1H), 7.88 (d, J=9.2 Hz, 1H), 7.45 (d, J=8.9 Hz, 1H), 7.38 (s, 1H), 7.02-6.96 (m, 1H), 6.96-6.89 (m, 2H), 6.71 (s, 1H), 5.38-5.25 (m, 1H), 4.00 (q, J=6.9 Hz, 2H), 3.32-3.26 (m, 6H), 3.17-3.13 (m, 1H), 2.95-2.92 (m, 5H), 2.70-2.54 (m, 10H), 2.28-2.24 (m, 3H), 1.98 (d, J=13.3 Hz, 6H), 1.96-1.93 (m, 1H), 1.83 (d, J=10.2 Hz, 2H), 1.53 (m, 2H), 1.32 (t, J=7.6 Hz, 3H), 1.25 (t, J=6.9 Hz, 3H), 1.09 (m, 4H), 0.71 (s, 3H). [M+H]⁺=1046.7

### Example 469: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-methyl-2-oxobenzo[d]oxazol-3(2H)-yl)piperidine-2,6-dione

### Step 1: 7-(2-(benzyloxy)ethyl)-3-(2,6-bis(benzyloxy)pyridin-3-yl)-5-methylbenzo[d]oxazol-2(3H)-one

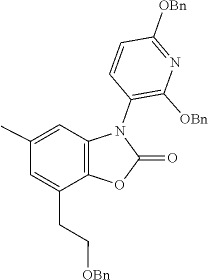 [SMILES: Cc1cc(CCOCc2ccccc2)c2oc(=O)n(-c3ccc(OCc4ccccc4)nc3OCc3ccccc3)c2c1]

To a solution of 3-(2,6-bis(benzyloxy)pyridin-3-yl)-7-bromo-5-methylbenzo[d]oxazol-2(3H)-one (1 g, 1.933 mmol, the compound was obtained by the way similar to example 505), [(2-bromoethoxy)methyl]benzene (0.54 g, 2.51 mmol) and benzamidine (0.05 g, 0.387 mmol) in DMA(10 mL) were added Mn (0.37 g, 6.77 mmol), nickel(II) iodide (0.18 g, 0.58 mmol), NaI (0.06 g, 0.39 mmol) and TFA (0.11 g, 0.97 mmol) at 0° C. The mixture was stirred for 15 min at room temperature and then 2 h at 100° C. under nitrogen atmosphere. The reaction was quenched with H₂O (150 mL) at room temperature. The resulting mixture was filtered, and the solid was washed with EA (50 mL). The filtrate was extracted with EA (50 mL×3). The combined organic layers were washed with brine (100 mL×3), dried over Na₂SO₄, filtered and concentrated under reduced pressure. The residue was applied onto a silica gel column with PE: EA (100: 0˜ 70: 30). This resulted in 7-(2-(benzyloxy)ethyl)-3-(2,6-bis(benzyloxy)pyridin-3-yl)-5-methylbenzo[d]oxazol-2(3H)-one (500 mg, 45.2%), [M+H]⁺=573.2.

### Step 2: 3-(7-(2-hydroxyethyl)-5-methyl-2-oxobenzo[d]oxazol-3(2H)-yl)piperidine-2,6-dione

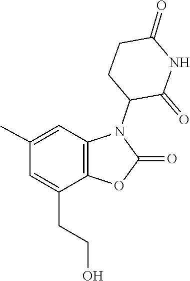 [SMILES: Cc1cc(CCO)c2oc(=O)n(C3CCC(=O)NC3=O)c2c1]

Into a 100-mL round-bottom flask was placed 7-(2-(benzyloxy)ethyl)-3-(2,6-bis(benzyloxy)pyridin-3-yl)-5-methylbenzo[d]oxazol-2(3H)-one (497 mg, 0.87 mmol), THF (15 mL) and Pd/C (500 mg, 10 wt. %, wet). The resulting solution was stirred overnight at room temperature under hydrogen atmosphere. The solids were filtered out. The resulting mixture was concentrated under vacuum. This resulted in 196 mg (74.0%) of 3-(7-(2-hydroxyethyl)-5-methyl-2-oxobenzo[d]oxazol-3(2H)-yl)piperidine-2,6-dione[M+H]⁺=305.2.

¹H NMR (500 MHz, DMSO) δ 11.18 (s, 1H), 6.95 (s, 1H), 6.85 (s, 1H), 5.35-5.29 (m, 1H), 4.75 (t, J=5.4 Hz, 1H), 3.69-3.63 (m, 2H), 2.99-2.57 (m, 5H), 2.30 (s, 3H), 2.21-2.02 (m, 1H).

### Step 3: 2-(3-(2,6-dioxopiperidin-3-yl)-5-methyl-2-oxo-2,3-dihydrobenzo[d]oxazol-7-yl)acetaldehyde

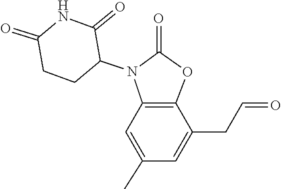

The title compound (48 mg, 89%) was prepared in a manner similar to that in Example 448 step 3 from 3-(7-(2-hydroxyethyl)-5-methyl-2-oxobenzo[d]oxazol-3(2H)-yl)piperidine-2,6-dione and DMP.

### Step 4: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-methyl-2-oxobenzo[d]oxazol-3(2H)-yl)piperidine-2,6-dione

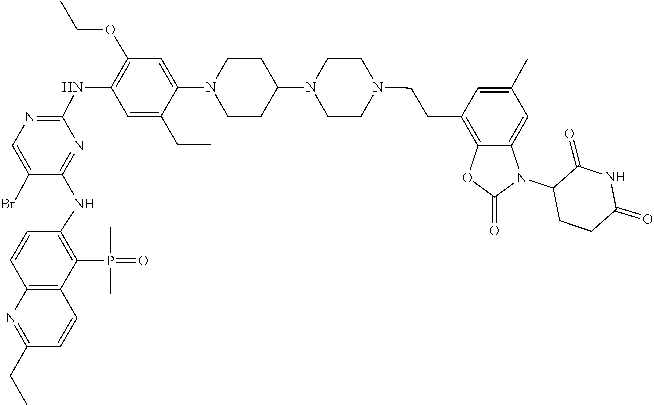 [SMILES: CCOc1cc(N2CCC(N3CCN(CCc4cc(C)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound (30 mg, 46.8%) was prepared in a manner similar to that in Example 492 step 6 from (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide and 2-(3-(2,6-dioxopiperidin-3-yl)-5-methyl-2-oxo-2,3-dihydrobenzo[d]oxazol-7-yl)acetaldehyde. ¹H NMR (500 MHz, DMSO) δ 11.73 (s, 1H), 11.19 (s, 1H), 8.60 (d, J=9.0 Hz, 1H), 8.22 (s, 2H), 7.91 (s, 2H), 7.45 (d, J=8.9 Hz, 1H), 7.38 (s, 1H), 6.95 (s, 1H), 6.86 (s, 1H), 6.71 (s, 1H), 5.33 (dd, J=13.1, 5.3 Hz, 1H), 4.00 (d, J=7.0 Hz, 2H), 2.99-2.88 (m, 6H), 2.81 (s, 2H), 2.77-2.60 (m, 5H), 2.59-2.51 (m, 2H), 2.49-2.40 (m, 5H), 2.31-2.25 (m, 7H), 2.19-2.09 (m, 1H), 1.98 (d, J=13.3 Hz, 6H), 1.83 (d, J=10.8 Hz, 2H), 1.53 (dd, J=20.1, 11.1 Hz, 2H), 1.32 (t, J=7.6 Hz, 3H), 1.25 (t, J=6.9 Hz, 3H), 0.71 (s, 3H). [M+H]⁺=1021.4

### Example 470: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-4-methyl-2-oxobenzo[d]oxazol-3(2H)-yl)piperidine-2,6-dione

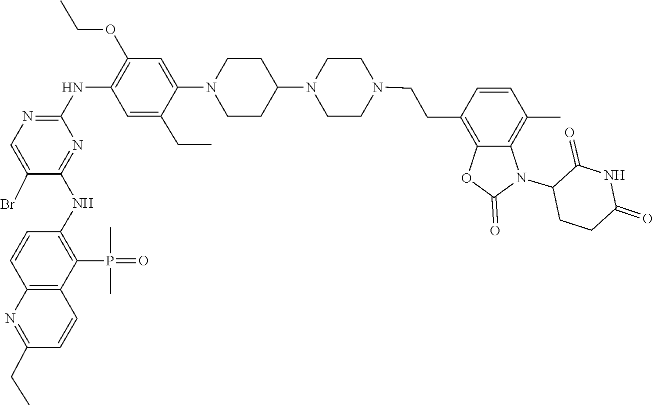 [SMILES: CCOc1cc(N2CCC(N3CCN(CCc4ccc(C)c5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound (28 mg, 37%) was prepared in a manner similar to that in Example 492. ¹H NMR (500 MHz, DMSO) δ 11.65 (s, 1H), 11.10 (s, 1H), 8.53 (d, J=8.9 Hz, 1H), 8.17 (s, 1H), 8.15 (s, 1H), 7.95-7.69 (m, 2H), 7.38 (d, J=8.9 Hz, 1H), 7.32 (s, 1H), 6.88 (q, J=8.1 Hz, 2H), 6.64 (s, 1H), 5.31 (dd, J=12.0, 5.5 Hz, 1H), 3.93 (q, J=6.9 Hz, 2H), 2.86 (dd, J=15.0, 7.5 Hz, 6H), 2.80-2.69 (m, 3H), 2.56 (t, J=12.9 Hz, 6H), 2.41 (s, 6H), 2.30-2.12 (m, 6H), 1.91 (d, J=13.3 Hz, 7H), 1.75 (d, J=10.9 Hz, 2H), 1.45 (d, J=9.1 Hz, 2H), 1.25 (t, J=7.6 Hz, 3H), 1.19 (t, J=6.9 Hz, 3H), 0.64 (s, 3H). [M+H]⁺=1021.4

### Example 471: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-6-fluoro-2-oxobenzo[d]oxazol-3(2H)-yl)piperidine-2_6-dione

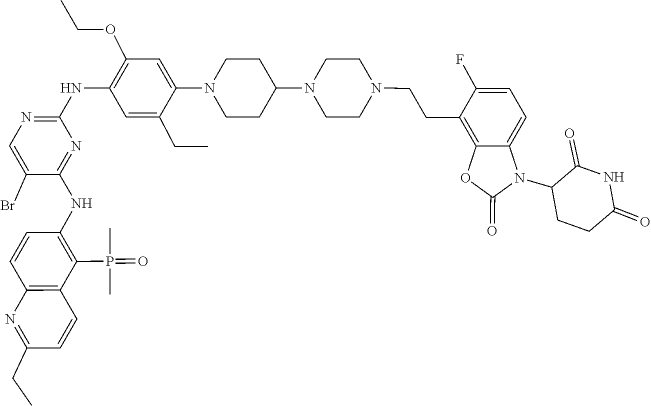 [SMILES: CCOc1cc(N2CCC(N3CCN(CCc4c(F)ccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound (12 mg, 26%) was prepared in a manner similar to that in Example 492. ¹H NMR (500 MHz, DMSO) δ 11.73 (s, 1H), 11.22 (s, 1H), 8.59 (d, J=8.8 Hz, 1H), 8.22 (d, J=4.8 Hz, 2H), 7.91 (s, 1H), 7.87 (d, J=9.3 Hz, 1H), 7.45 (d, J=8.9 Hz, 1H), 7.38 (s, 1H), 7.14 (dd, J=8.7, 4.4 Hz, 1H), 7.07 (d, J=8.7 Hz, 1H), 6.71 (s, 1H), 5.40-5.32 (m, 1H), 4.00 (q, J=7.0 Hz, 2H), 2.97-2.83 (m, 8H), 2.73-2.57 (m, 7H), 2.57-2.51 (m, 6H), 2.30-2.20 (m, 3H), 2.16 (dd, J=10.2, 5.0 Hz, 1H), 1.98 (d, J=13.3 Hz, 6H), 1.82 (d, J=11.6 Hz, 2H), 1.52 (d, J=8.9 Hz, 2H), 1.32 (t, J=7.6 Hz, 3H), 1.25 (t, J=6.9 Hz, 3H), 0.71 (s, 3H); [M+H]⁺=1025.5.

### Example 472: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-fluoro-2-oxobenzo[d]oxazol-3(2H)-yl)piperidine-2,6-dione

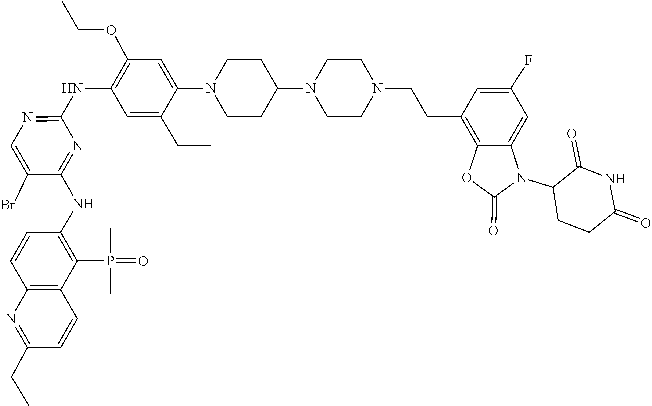 [SMILES: CCOc1cc(N2CCC(N3CCN(CCc4cc(F)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound (17.41 mg, 36%) was prepared in a manner similar to that in Example 492. ¹H NMR (500 MHz, DMSO) δ 11.73 (s, 1H), 11.21 (s, 1H), 8.59 (d, J=8.8 Hz, 1H), 8.24 (d, J=16.3 Hz, 2H), 7.96-7.78 (m, 2H), 7.45 (d, J=8.9 Hz, 1H), 7.38 (s, 1H), 7.16 (dd, J=8.5, 2.5 Hz, 1H), 6.97 (dd, J=10.7, 2.5 Hz, 1H), 6.71 (s, 1H), 5.35 (dd, J=13.0, 5.4 Hz, 1H), 4.00 (q, J=7.0 Hz, 2H), 2.93 (dd, J=15.0, 7.5 Hz, 4H), 2.86 (t, J=7.3 Hz, 3H), 2.76-2.68 (m, 1H), 2.68-2.61 (m, 3H), 2.58 (dd, J=14.3, 6.6 Hz, 3H), 2.53-2.51 (m, 1H), 2.51-2.50 (m, 3H), 2.49-2.46 (m, 3H), 2.32-2.20 (m, 3H), 2.20-2.09 (m, 1H), 1.99 (s, 3H), 1.97 (s, 3H), 1.82 (d, J=11.9 Hz, 2H), 1.56-1.51 (m, 2H), 1.32 (t, J=7.6 Hz, 3H), 1.25 (t, J=6.9 Hz, 3H), 0.71 (s, 3H). [M+H]⁺=1025.6.

### Example 473: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

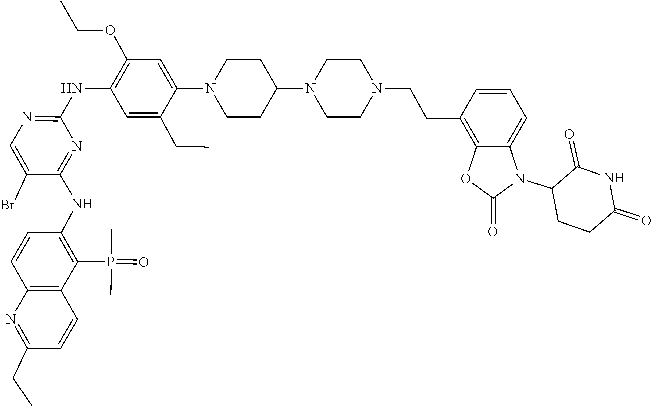 [SMILES: CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

A mixture of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (75 mg, 0.10 mmol) and 2-(3-(2,6-dioxopiperidin-3-yl)-2-oxo-2,3-dihydrobenzo[d]oxazol-7-yl)acetaldehyde (35 mg, 0.12 mmol) in DCM (8 mL) was stirred in a flask at room temperature for 10 min. To the mixture was added sodium triacetoxyborohydride (65 mg, 0.31 mmol) and the reaction was stirred at room temperature for another 0.5 h. Then the mixture was concentrated under vacuum to afford the crude product, which was purified with prep-HPLC chromatography (0.1% FA in water: acetonitrile=90:10˜50:50 gradient elution) to give the product (31 mg, 30.0%). ¹H NMR (500 MHz, DMSO) δ 11.72 (s, 1H), 11.21 (s, 1H), 8.59 (d, J=9.0 Hz, 1H), 8.24 (s, 1H), 8.22 (s, 1H), 7.91 (s, 1H), 7.88 (d, J=9.3 Hz, 1H), 7.45 (d, J=8.9 Hz, 1H), 7.38 (s, 1H), 7.13-7.09 (m, 3H), 6.71 (s, 1H), 5.36 (dd, J=12.9, 5.3 Hz, 1H), 4.00 (q, J=7.0 Hz, 2H), 3.29-3.21 (m, 4H), 2.93-2.87 (m, 7H), 2.74-2.52 (m, 10H), 2.28-2.25 (m, 3H), 2.19-2.12 (m, 1H), 1.98 (d, J=13.3 Hz, 6H), 1.83 (d, J=11.7 Hz, 2H), 1.53 (dd, J=20.3, 11.3 Hz, 2H), 1.32 (t, J=7.6 Hz, 3H), 1.25 (t, J=6.9 Hz, 3H), 0.71 (s, 3H). [M+H]⁺=1007.7

### Example 474: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-4,5-difluoro-2-oxobenzo[d]oxazol-3(2H)-yl)piperidine-2,6-dione

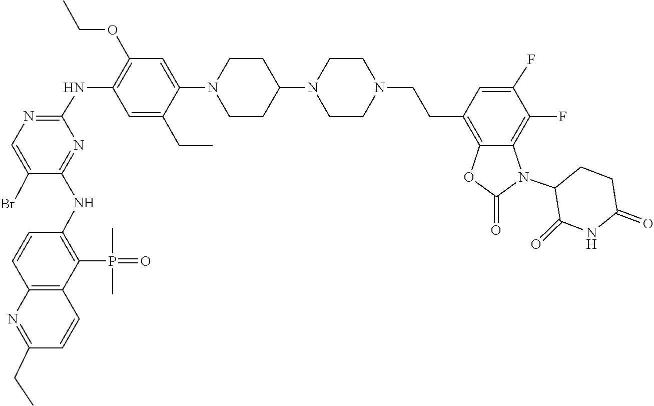 [SMILES: CCOc1cc(N2CCC(N3CCN(CCc4cc(F)c(F)c5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound (16.4 mg, 32.1%) was prepared in a manner similar to that in Example 492. ¹H NMR (500 MHz, DMSO) δ 11.73 (s, 1H), 11.28 (s, 1H), 8.60 (d, J=9.0 Hz, 1H), 8.23 (d, J=11.1 Hz, 2H), 7.96-7.74 (m, 2H), 7.45 (d, J=8.9 Hz, 1H), 7.38 (s, 1H), 7.25 (dd, J=12.5, 7.5 Hz, 1H), 6.71 (s, 1H), 4.02-3.98 (m, 2H), 3.29 (s, 4H), 2.95-2.91 (m, 5H), 2.84-2.82 (m, 2H), 2.75-2.55 (m, 9H), 2.30-2.26 (m, 6H), 1.99 (s, 3H), 1.97 (s, 3H), 1.82 (s, 2H), 1.55-1.52 (m, 2H), 1.33-1.31 (m, 3H), 1.28-1.24 (m, 3H), 0.71 (s, 3H). [M+H]⁺=1043.8.

### Example 476: 3-(7-((S)-3-((4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)methyl)pyrrolidin-1-yl)-5-fluoro-2-oxobenzo[d]oxazol-3(2H)-yl)piperidine-2,6-dione

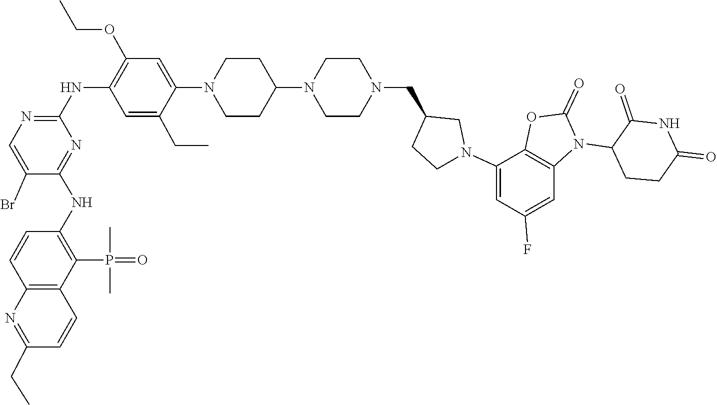 [SMILES: CCOc1cc(N2CCC(N3CCN(C[C@@H]4CCN(c5cc(F)cc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound (15.23 mg, 30%) was prepared in a manner similar to that in Example 492. ¹H NMR (500 MHz, DMSO) δ 11.73 (s, 1H), 11.19 (s, 1H), 8.60 (d, J=8.7 Hz, 1H), 8.38 (s, 1H), 8.22 (s, 1H), 8.01-7.85 (m, 2H), 7.44 (d, J=8.9 Hz, 1H), 7.39 (s, 1H), 6.71 (s, 1H), 6.48 (dd, J=8.3, 2.3 Hz, 1H), 6.17 (dd, J=13.0, 2.3 Hz, 1H), 5.28 (dd, J=12.9, 5.3 Hz, 1H), 4.04-3.98 (m, 2H), 3.62-3.58 (m, 2H), 3.51-3.49 (m, 2H), 3.42-3.39 (m, 2H), 3.23-3.12 (m, 2H), 2.93 (dd, J=14.9, 7.4 Hz, 4H), 2.84 (s, 1H), 2.71-2.60 (m, 4H), 2.56-2.52 (m, 4H), 2.42-2.34 (m, 6H), 2.12 (s, 2H), 1.99 (s, 3H), 1.97 (s, 3H), 1.85-1.82 (m, 2H), 1.69-1.66 (m, 1H), 1.55-1.52 (m, 2H), 1.34-1.31 (m, 3H), 1.27-1.24 (m, 3H), 0.71 (s, 3H). [M+H]⁺=1080.9.

### Example 477: 3-(7-((R)-3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazine-1-carbonyl)pyrrolidin-1-yl)-5-fluoro-2-oxobenzo[d]oxazol-3(2H)-yl)piperidine-2,6-dione

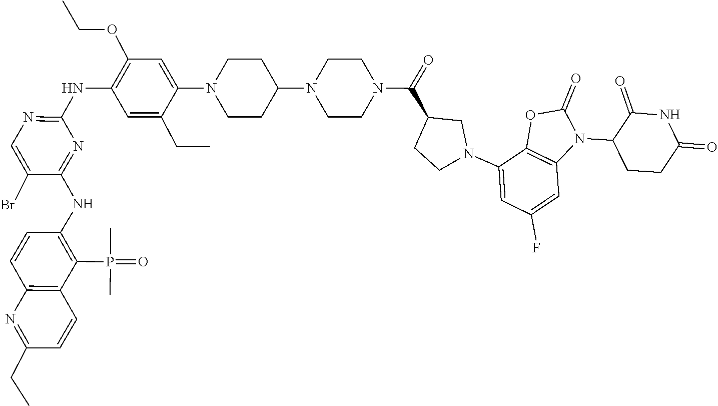 [SMILES: CCOc1cc(N2CCC(N3CCN(C(=O)[C@@H]4CCN(c5cc(F)cc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound (15.63 mg, 31%) was prepared in a manner similar to that in Example 447. ¹HNMR (500 MHz, DMSO) δ 11.73 (s, 1H), 11.19 (s, 1H), 8.60 (d, J=9.0 Hz, 1H), 8.23 (d, J=10.7 Hz, 2H), 7.99-7.78 (m, 2H), 7.54-7.28 (m, 2H), 6.71 (s, 1H), 6.52 (dd, J=8.3, 2.2 Hz, 1H), 6.22 (dd, J=12.9, 2.2 Hz, 1H), 5.28 (dd, J=12.9, 5.2 Hz, 1H), 4.02-3.98 (m, 2H), 3.76-3.48 (m, 9H), 3.29-3.27 (m, 2H), 2.97-2.91 (m, 4H), 2.87-2.81 (m, 1H), 2.68-2.63 (m, 4H), 2.56-2.53 (m, 2H), 2.36 (s, 1H), 2.25 (d, J=7.0 Hz, 2H), 2.22-2.01 (m, 3H), 2.00 (s, 3H), 1.98 (s, 3H), 1.83 (d, J=10.5 Hz, 2H), 1.57-1.55 (m, 2H), 1.32-1.28 (m, 6H), 0.71 (s, 3H). [M+H]⁺=1094.7.

### Example 478: 3-(7-((R)-3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazine-1-carbonyl)pyrrolidin-1-yl)-2-oxobenzo[d]oxazol-3(2H)-yl)piperidine-2,6-dione

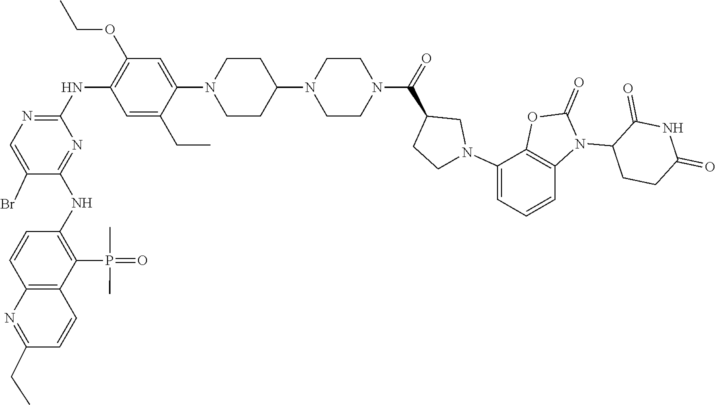 [SMILES: CCOc1cc(N2CCC(N3CCN(C(=O)[C@@H]4CCN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound (30 mg, 45%) was prepared in a manner similar to that in Example 447. ¹H NMR (500 MHz, DMSO) δ 11.79 (s, 1H), 11.19 (s, 1H), 8.56 (d, J=8.9 Hz, 1H), 8.27 (s, 1H), 8.21 (s, 1H), 8.00 (s, 1H), 7.88 (d, J=9.4 Hz, 1H), 7.44 (d, J=8.9 Hz, 1H), 7.33 (s, 1H), 7.02 (t, J=7.9 Hz, 1H), 6.76 (s, 1H), 6.54 (d, J=7.4 Hz, 1H), 6.40 (d, J=8.2 Hz, 1H), 5.30 (d, J=7.9 Hz, 1H), 3.76 (s, 3H), 3.71-3.44 (m, 11H), 2.94-2.90 (m, 6H), 2.67 (dd, J=22.2, 12.8 Hz, 5H), 2.57 (s, 2H), 2.35-2.31 (m, 3H), 2.16-2.11 (m, 3H), 1.98 (d, J=13.2 Hz, 6H), 1.84 (d, J=10.9 Hz, 2H), 1.57 (d, J=10.6 Hz, 2H), 1.32 (dd, J=8.6, 6.5 Hz, 3H), 0.77 (s, 3H). [M+H]⁺=1076.4

### Example 479: 3-(7-(3-((4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)methyl)azetidin-1-yl)-5-fluoro-2-oxobenzo[d]oxazol-3(2H)-yl)piperidine-2,6-dione

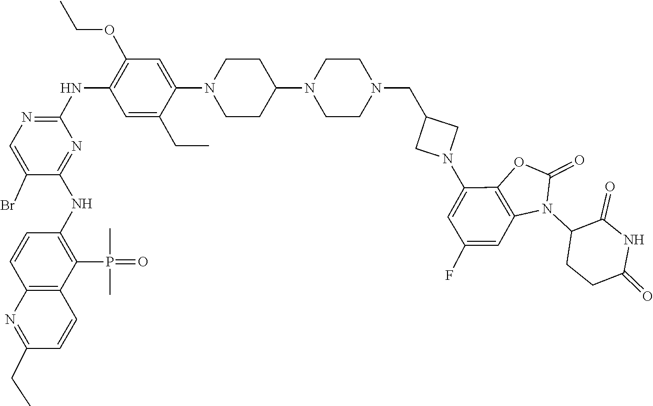 [SMILES: CCOc1cc(N2CCC(N3CCN(CC4CN(c5cc(F)cc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound (14.2 mg, 27.6%) was prepared in a manner similar to that in Example 492. ¹H NMR (500 MHz, DMSO) δ 11.73 (s, 1H), 11.18 (s, 1H), 8.60 (d, J=8.9 Hz, 1H), 8.23 (d, J=10.9 Hz, 2H), 8.00-7.64 (m, 2H), 7.53-7.21 (m, 2H), 6.71 (s, 1H), 6.56 (dd, J=8.5, 2.2 Hz, 1H), 6.10 (dd, J=11.9, 2.2 Hz, 1H), 5.28 (dd, J=12.9, 5.3 Hz, 1H), 4.13-4.11 (m, 2H), 4.01-3.99 (m, 2H), 3.69-3.67 (m, 2H), 2.95-2.92 (m, 5H), 2.86-2.82 (m, 1H), 2.68-2.65 (m, 5H), 2.57 (d, J=7.3 Hz, 2H), 2.54-2.51 (m, 3H), 2.40 (s, 4H), 2.31-2.19 (m, 3H), 2.11-2.09 (m, 1H), 1.98 (d, J=13.3 Hz, 6H), 1.82 (d, J=10.9 Hz, 2H), 1.53-1.51 (m, 2H), 1.33-1.31 (m, 3H), 1.27-1.24 (m, 3H), 0.71 (s, 3H). [M+H]⁺=1066.6.

### Example 480: 3-(7-((R)-4-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazine-1-carbonyl)-3,3-dimethylpyrrolidin-1-yl)-5-fluoro-2-oxobenzo[d]oxazol-3(2H)-yl)piperidine-2,6-dione

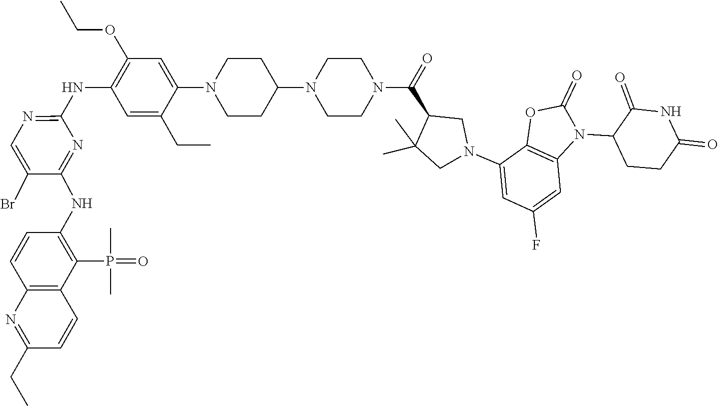 [SMILES: CCOc1cc(N2CCC(N3CCN(C(=O)[C@H]4CN(c5cc(F)cc6c5oc(=O)n6C5CCC(=O)NC5=O)CC4(C)C)CC3)CC2)c(CC)cc1Nc1ncc(Br)c(Nc2ccc3nc(CC)ccc3c2P(C)(C)=O)n1]

The title compound (22.05 mg, 42.1%) was prepared in a manner similar to that in Example 447. ¹H NMR (500 MHz, DMSO) δ 11.72 (s, 1H), 11.18 (s, 1H), 8.60 (d, J=8.9 Hz, 1H), 8.23 (d, J=11.0 Hz, 2H), 7.98-7.80 (m, 2H), 7.45 (d, J=8.9 Hz, 1H), 7.38 (s, 1H), 6.71 (s, 1H), 6.49-6.47 (m, 1H), 6.14 (dd, J=13.0, 2.1 Hz, 1H), 5.28-5.26 (m, 1H), 4.01-3.99 (m, 2H), 3.70 (d, J=5.7 Hz, 2H), 3.60 (s, 2H), 3.52 (s, 2H), 3.36 (s, 3H), 3.30-3.22 (m, 2H), 2.92 (s, 4H), 2.86-2.82 (m, 1H), 2.69-2.66 (m, 4H), 2.56-2.52 (m, 2H), 2.34 (d, J=11.8 Hz, 1H), 2.25 (d, J=6.7 Hz, 2H), 2.19-2.06 (m, 1H), 1.99 (s, 3H), 1.97 (s, 3H), 1.84-1.82 (m, 2H), 1.57-1.54 (m, 2H), 1.32 (t, J=7.6 Hz, 3H), 1.25 (s, 3H), 1.21 (s, 3H), 1.01 (s, 3H), 0.71 (s, 3H). [M+H]⁺=1121.9.

### Example 481: 3-(7-(4-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazine-1-carbonyl)piperidin-1-yl)-2-oxobenzo[d]oxazol-3(2H)-yl)piperidine-2,6-dione

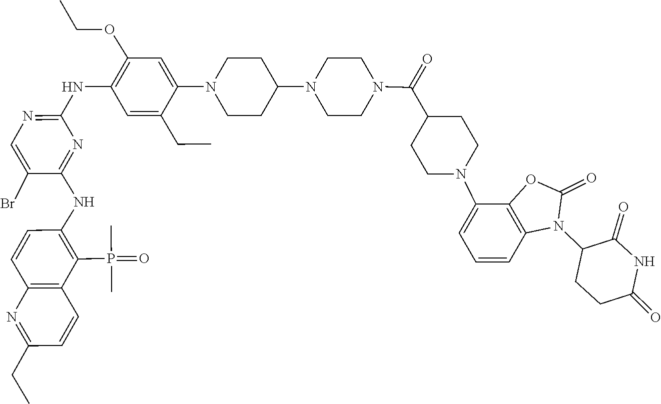 [SMILES: CCOc1cc(N2CCC(N3CCN(C(=O)C4CCN(c5cccc6c5oc(=O)n6C5CCC(=O)NC5=O)CC4)CC3)CC2)c(CC)cc1Nc1ncc(Br)c(Nc2ccc3nc(CC)ccc3c2P(C)(C)=O)n1]

The title compound (50 mg, 65%) was prepared in a manner similar to that in Example 447. ¹H NMR (500 MHz, DMSO) δ 11.73 (s, 1H), 11.20 (s, 1H), 8.60 (d, J=9.2 Hz, 1H), 8.35-8.18 (m, 2H), 7.91 (s, 1H), 7.88 (d, J=9.3 Hz, 1H), 7.45 (d, J=8.9 Hz, 1H), 7.39 (s, 1H), 7.09 (t, J=8.1 Hz, 1H), 6.77 (d, J=7.8 Hz, 1H), 6.72 (d, J=7.4 Hz, 2H), 5.33 (dd, J=12.9, 5.3 Hz, 1H), 4.00 (q, J=6.9 Hz, 2H), 3.71 (d, J=11.9 Hz, 2H), 3.55 (s, 2H), 3.48 (s, 2H), 2.97-2.82 (m, 8H), 2.69-2.65 (m, 5H), 2.54 (s, 2H), 2.36 (s, 1H), 2.26 (s, 2H), 2.15 (d, J=5.4 Hz, 1H), 1.98 (d, J=13.3 Hz, 7H), 1.82 (d, J=11.4 Hz, 2H), 1.73 (s, 4H), 1.56 (d, J=8.8 Hz, 2H), 1.32 (s, 3H), 1.26 (s, 3H), 0.71 (s, 3H). [M+H]⁺=1090.4.

### Example 482: 3-(4-(2-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

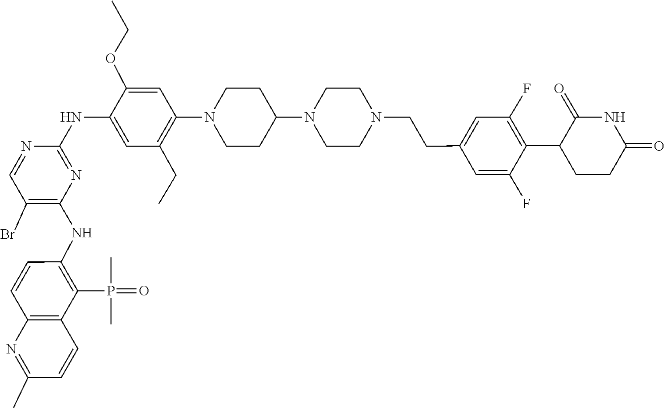 [SMILES: CCOc1cc(N2CCC(N3CCN(CCc4cc(F)c(C5CCC(=O)NC5=O)c(F)c4)CC3)CC2)c(CC)cc1Nc1ncc(Br)c(Nc2ccc3nc(C)ccc3c2P(C)(C)=O)n1]

The title compound (23.56 mg, 17.3%) was prepared in a manner similar to that in Example 484. ¹H NMR (500 MHz, DMSO) δ 11.70 (s, 1H), 10.95 (s, 1H), 8.59 (d, J=9.0 Hz, 1H), 8.27 (br s, 1H), 8.22 (s, 1H), 7.95-7.81 (m, 2H), 7.48-7.38 (m, 2H), 7.03 (d, J=10.0 Hz, 2H), 6.70 (s, 1H), 4.20 (dd, J=13.0, 5.0 Hz, 1H), 4.00 (q, J=7.0 Hz, 2H), 2.91 (d, J=11.0 Hz, 2H), 2.86-2.71 (m, 4H), 2.69-2.50 (m, 14H), 2.30-2.19 (m, 3H), 2.13-2.11 (m, 1H), 2.03-1.90 (m, 8H), 1.82 (d, J=11.0 Hz, 2H), 1.56-1.50 (m, 2H), 1.27-1.20 (m, 6H). [M+H]⁺=972.5.

### Example 483: (R)-3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

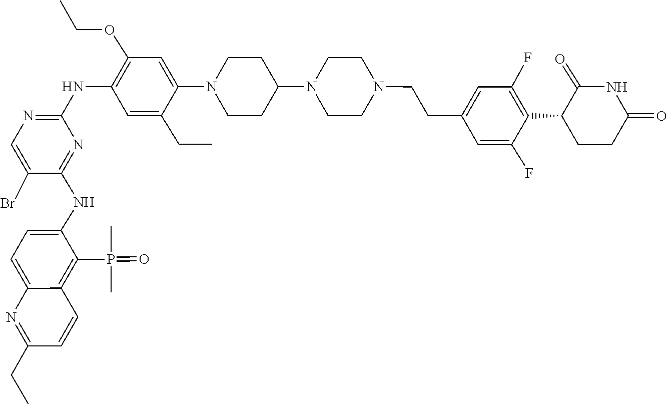 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)ccc3c2P(C)(C)=O)n1]

A mixture of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (75 mg, 0.10 mmol) and (R)-2-(4-(2,6-dioxopiperidin-3-yl)-3,5-difluorophenyl)acetaldehyde (32 mg, 0.12 mmol) in DCM (8 mL) was stirred in a flask at room temperature for 10 min. To the mixture was added sodium triacetoxyborohydride (65 mg, 0.31 mmol) and the reaction was stirred at room temperature for another 0.5 h. Then the mixture was concentrated under vacuum to afford the crude product, which was purified with prep-HPLC chromatography (0.1% FA in water: acetonitrile=90:10˜ 50:50 gradient elution) to give the product (29 mg, 29.2%). ¹H NMR (500 MHz, DMSO) δ 11.74 (s, 1H), 10.97 (s, 1H), 8.60 (d, J=8.9 Hz, 1H), 8.23 (d, J=6.2 Hz, 2H), 7.93 (s, 1H), 7.87 (d, J=9.3 Hz, 1H), 7.45 (d, J=8.9 Hz, 1H), 7.38 (s, 1H), 7.03 (d, J=10.1 Hz, 2H), 6.70 (s, 1H), 4.20 (dd, J=12.7, 5.0 Hz, 1H), 4.00 (q, J=6.9 Hz, 2H), 3.33-3.25 (m, 2H), 2.95-2.91 (m, 4H), 2.86-2.73 (m, 3H), 2.66-2.63 (m, 7H), 2.48-2.44 (m, 4H), 2.24 (d, J=7.2 Hz, 3H), 2.15-2.12 (m, 1H), 2.03-1.96 (m, 7H), 1.82 (d, J=11.1 Hz, 2H), 1.60-1.44 (m, 2H), 1.32 (t, J=7.6 Hz, 3H), 1.25 (t, J=6.9 Hz, 3H), 0.71 (s, 3H).[M+H]⁺=986.7.

### Example 485: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

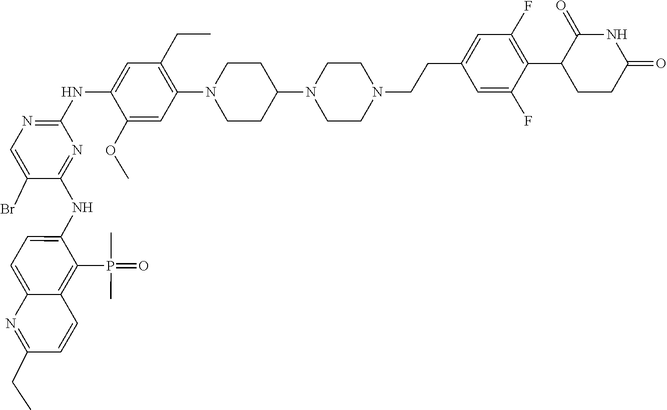 [SMILES: CCc1ccc2c(P(C)(C)=O)c(Nc3nc(Nc4cc(CC)c(N5CCC(N6CCN(CCc7cc(F)c(C8CCC(=O)NC8=O)c(F)c7)CC6)CC5)cc4OC)ncc3Br)ccc2n1]

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (500 mg, 0.694 mmol) and 2-(4-(2,6-dioxopiperidin-3-yl)-3,5-difluorophenyl)acetaldehyde (222.49 mg, 0.832 mmol) in DCM (8 mL) was stirred in a flask at room temperature for 2 hour. To the mixture was added sodium triacetoxyborohydride (146.34 mg, 0.694 mmol) and the reaction was stirred at room temperature for another 2 h. The resulting mixture was diluted with H₂O (60 mL) and the layers were separated. The aqueous layer was extracted with DCM (3×30 mL). The combined organic layers were washed with brine (50 mL×3), dried over anhydrous Na₂SO₄, filtered and concentrated under reduced pressure. The residue was purified by silica gel column chromatography to afford the crude product (600 mg), which was purified with prep-HPLC chromatography (0.1% FA in water: acetonitrile=90:10˜ 50:50 gradient elution) to give the product (450 mg, 66.7%). ¹H NMR (500 MHz, DMSO) δ 11.73 (s, 1H), 10.88 (s, 1H), 8.49 (d, J=8.8 Hz, 1H), 8.20 (s, 1H), 8.15 (d, J=12.4 Hz, 1H), 7.94 (s, 1H), 7.80 (d, J=9.4 Hz, 1H), 7.37 (d, J=8.9 Hz, 1H), 7.26 (s, 1H), 6.96 (d, J=10.0 Hz, 2H), 6.68 (s, 1H), 4.13 (dd, J=12.6, 5.0 Hz, 1H), 3.69 (s, 3H), 2.86 (dd, J=15.2, 7.6 Hz, 4H), 2.79-2.65 (m, 4H), 2.59 (t, J=11.3 Hz, 3H), 2.47 (s, 4H), 2.41-2.31 (m, 4H), 2.22 (d, J=4.7 Hz, 3H), 2.05 (s, 2H), 1.92 (d, J=13.3 Hz, 7H), 1.77 (d, J=10.2 Hz, 2H), 1.47 (d, J=8.8 Hz, 2H), 1.25 (t, J=7.6 Hz, 3H), 0.70 (s, 3H). [M+H]⁺=972.7.

### Example 487: (R)-3-(4-((R)-4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

### Step 1: methyl (R)-4,4-dimethylpyrrolidine-3-carboxylate

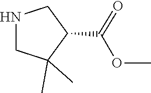

To a stirred solution of (R)-4,4-dimethylpyrrolidine-3-carboxylic acid (2 g, 13.96 mmol) in MeOH (30 mL) was added SOCl₂ (1.66 g, 13.96 mmol) dropwise at 0° C. under nitrogen atmosphere. The resulting mixture was stirred for 2 hrs at 60° C. temperature. The resulting mixture was concentrated under reduced pressure to afford the product (2.1 g, 95.8%) which was used for next step without further purification. [M+H]⁺=158.1

### Step 2: methyl (R)-1-(4-(2,6-bis(benzyloxy)pyridin-3-yl)-3,5-difluorophenyl)-4,4-dimethylpyrrolidine-3-carboxylate

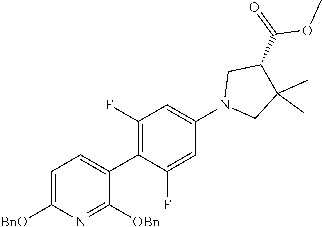 [SMILES: COC(=O)[C@H]1CN(c2cc(F)c(-c3ccc(OCc4ccccc4)nc3OCc3ccccc3)c(F)c2)CC1(C)C]

To a stirred solution of 2,6-bis(benzyloxy)-3-(4-bromo-2,6-difluorophenyl)pyridine (9.25 g, 20.25 mmol) and_methyl (R)-4,4-dimethylpyrrolidine-3-carboxylate (2.1 g, 13.35 mmol) in dioxane (50 mL) were added Cs₂CO₃ (10.95 g, 33.37 mmol), Xantphos (1.54 g, 2.67 mmol) and Pd₂(dba)₃ (1.22 g, 1.35 mmol) at room temperature under nitrogen atmosphere. The resulting mixture was stirred for 16 h at 100° C. under nitrogen atmosphere. The mixture was allowed to cool down to room temperature. The resulting mixture was concentrated under reduced pressure. The residue was diluted with EtOAc (500 mL), washed with water (3×200 mL) and brine (200 mL). The organic layer was dried over anhydrous Na₂SO₄, filtered and concentrated under reduced pressure. The residue was purified by silica gel column chromatography, eluted with PE/EtOAc (5: 1) to afford the product (4.5 g, 60.8%); [M+H]⁺=559.6

### Step 3: (R)-1-(4-(2,6-bis(benzyloxy)pyridin-3-yl)-3,5-difluorophenyl)-4,4-dimethylpyrrolidine-3-carboxylic acid

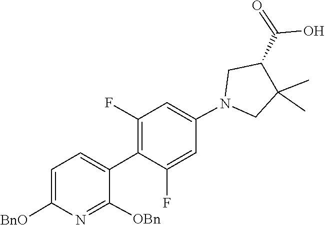 [SMILES: CC1(C)CN(c2cc(F)c(-c3ccc(OCc4ccccc4)nc3OCc3ccccc3)c(F)c2)C[C@@H]1C(=O)O]

To a solution of methyl (R)-1-(4-(2,6-bis(benzyloxy)pyridin-3-yl)-3,5-difluorophenyl)-4,4-dimethylpyrrolidine-3-carboxylate (4.5 g, 8.05 mmol) in THF (40 mL) and H₂O (10 mL) was added lithium hydroxide hydrate (337.9 mg, 8.05 mmol) at 25° C. The resulting mixture was stirred at 25° C. for 5 h. The reaction was quenched with HCl (1 N) at 0° C. until pH=6 and the resulting mixture was extracted with EA (2×40 mL). The combined organic layers were washed with brine, dried over anhydrous Na₂SO₄, filtered and concentrated under vacuum to afford the crude product (4.05 g, 92.46%), which was used for next step without further purification. [M+H]⁺=545.6

### Step 4: (R)-1-(4-((R)-2,6-dioxopiperidin-3-yl)-3,5-difluorophenyl)-4,4-dimethylpyrrolidine-3-carboxylic acid

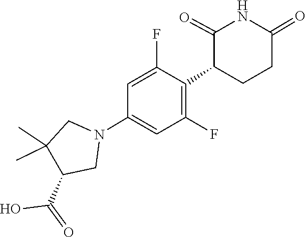 [SMILES: CC1(C)CN(c2cc(F)c([C@H]3CCC(=O)NC3=O)c(F)c2)C[C@@H]1C(=O)O]

(R)-1-(4-(2,6-bis(benzyloxy)pyridin-3-yl)-3,5-difluorophenyl)-4,4-dimethylpyrrolidine-3-carboxylic acid (4.5 g, 8.25 mmol) was dissolved in DCM (30 mL) and iPr-OH (30 mL). Pd/C (1 g, 10 wt. %, wet) was added to the solution in one portion. The resulting mixture was stirred under hydrogen atmosphere (1 atm) at room temperature overnight. The solid was filtered off and the filtrate was concentrated to give the crude product. The crude was triturated with MTBE to give the desired product which was purified by HPLC (IF (2*25 cm, 5 um), 60% MtBE/40% MeOH:DCM=1:1, 80 bar, 20 ml/min) and corresponded to peak A @ 1.216 min/254 nm (1.13 g, 25%). [M+H]⁺=367.4.

### Step 5: (R)-3-(4-((R)-4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

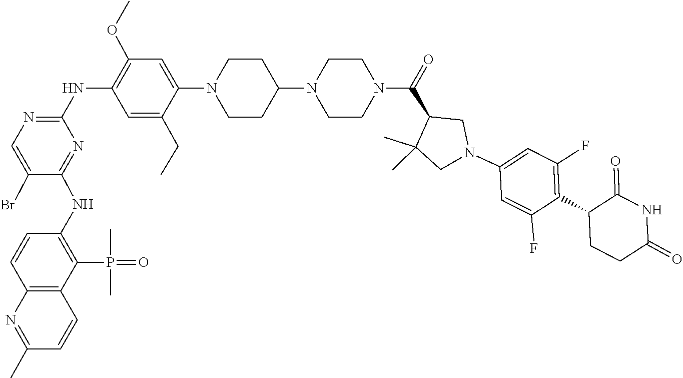 [SMILES: CCc1cc(Nc2ncc(Br)c(Nc3ccc4nc(C)ccc4c3P(C)(C)=O)n2)c(OC)cc1N1CCC(N2CCN(C(=O)[C@H]3CN(c4cc(F)c([C@H]5CCC(=O)NC5=O)c(F)c4)CC3(C)C)CC2)CC1]

A mixture of(6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (50 mg, 0.0708 mmol), (R)-1-(4-((R)-2,6-dioxopiperidin-3-yl)-3,5-difluorophenyl)-4,4-dimethylpyrrolidine-3-carboxylic acid (28.5 mg, 0.0779 mmol), T3P (67.45 mg, 0.212 mmol) and DIEA (27.39 mg, 0.212 mmol) in DCM (4 mL) was stirred in a flask at room temperature for 1 hours. The mixture was diluted with water (20 mL), and the layers were separated. The aqueous layer was extracted with DCM (20 mL×3). The combined organic layers were washed with brine (20 mL×3), dried over Na₂SO₄, filtered and concentrated under reduced pressure. The crude (30 mg) product was purified with prep-HPLC chromatography (0.1% FA in water: acetonitrile=90:10˜ 50:50 gradient elution) to give the product (30 mg, 40.21%). ¹H NMR (500 MHz, DMSO) δ 11.76 (s, 1H), 10.84 (s, 1H), 8.56 (d, J=8.9 Hz, 1H), 8.30 (s, 1H), 8.21 (s, 1H), 7.98 (s, 1H), 7.87 (d, J=9.3 Hz, 1H), 7.42 (d, J=8.9 Hz, 1H), 7.38 (s, 1H), 6.74 (s, 1H), 6.15 (d, J=12.2 Hz, 2H), 4.01 (dd, J=12.4, 5.0 Hz, 1H), 3.76 (s, 3H), 3.46-3.41 (m, 8H), 3.09 (dd, J=18.7, 9.1 Hz, 2H), 2.95 (d, J=10.7 Hz, 2H), 2.84-2.72 (m, 1H), 2.65 (s, 5H), 2.54 (s, 2H), 2.41-2.24 (m, 3H), 2.08 (dd, J=23.4, 13.7 Hz, 1H), 1.98 (d, J=13.3 Hz, 8H), 1.83 (d, J=11.2 Hz, 2H), 1.57 (d, J=11.0 Hz, 2H), 1.23 (s, 1H), 1.18 (s, 3H), 0.99 (s, 3H), 0.77 (s, 3H). [M+H]⁺=1055.4.

### Example 490: 3-(6-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

### Step 1: 2-(3-(2,6-dioxopiperidin-3-yl)-2-oxo-2,3-dihydrobenzo[d]oxazol-6-yl)acetaldehyde

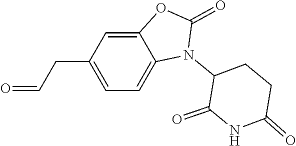 [SMILES: O=CCc1ccc2c(c1)oc(=O)n2C1CCC(=O)NC1=O]

A mixture of 3-(6-(2-hydroxyethyl)-2-oxobenzo[d]oxazol-3(2H)-yl)piperidine-2,6-dione (4 g, 13.37 mmol) and DMP (8.76 g, 20.68 mmol) in DCM (50 mL) and THF (50 mL) was stirred in a flask at room temperature for 4 h. The reaction was quenched with water (200 mL) and the layers were separated. The aqueous layer was extracted with EtOAc (50 mL×3). The combined organic layers were washed with saturated aqueous NaCl (50 mL×3) and saturated aqueous NaHCO₃(50 mL×2), dried over anhydrous Na₂SO₄, filtered and concentrated in vacuum to afford the product (2.0 g, 51.8%). [M+H]⁺=289.1.

### Step 2: 3-(6-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

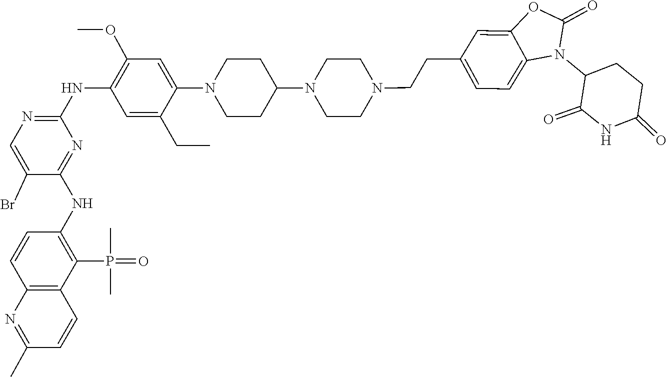 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)oc(=O)n4C3CCC(=O)NC3=O)CC2)CC1]

A mixture of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (50 mg, 0.0708 mmol) and 2-(3-(2,6-dioxopiperidin-3-yl)-2-oxo-2,3-dihydrobenzo[d]oxazol-6-yl)acetaldehyde (24.48 mg, 0.0849 mmol) in DCM (8 mL) was stirred in a flask at room temperature for 2 hour. To the mixture was added NaBH₃CN (14.9 mg, 0.0708 mmol) and the reaction was stirred at room temperature for another 2 h. The resulting mixture was diluted with H₂O (30 mL), and the layers were separated. The aqueous layer was extracted with DCM (3×15 mL). The combined organic layers were washed with brine (20 mL×3), dried over anhydrous Na₂SO₄, filtered and concentrated under reduced pressure. The residue was purified by silica gel column chromatography to afford the crude product (30 mg), The crude (30 mg) product was purified with prep-HPLC chromatography (0.1% FA in water: acetonitrile=90:10˜ 50:50 gradient elution) to give the product (24 mg, 34.6%). ¹H NMR (500 MHz, DMSO) δ 11.69 (s, 1H), 11.14 (s, 1H), 8.50 (d, J=8.8 Hz, 1H), 8.23 (s, 1H), 8.15 (s, 1H), 7.91 (s, 1H), 7.80 (d, J=9.5 Hz, 1H), 7.38-7.29 (m, 2H), 7.24 (s, 1H), 7.10 (d, J=8.0 Hz, 1H), 7.02 (d, J=8.2 Hz, 1H), 6.67 (s, 1H), 5.28 (dd, J=12.9, 5.3 Hz, 1H), 3.69 (s, 3H), 2.88 (d, J=11.2 Hz, 4H), 2.72 (s, 3H), 2.68-2.60 (m, 4H), 2.58 (s, 7H), 2.51 (s, 4H), 2.23 (d, J=7.0 Hz, 2H), 2.12-2.05 (m, 1H), 1.91 (d, J=13.3 Hz, 7H), 1.80 (s, 2H), 1.51 (d, J=8.9 Hz, 2H), 0.70 (s, 3H). [M+H]⁺=979.3.

### Example 491: 3-(6-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

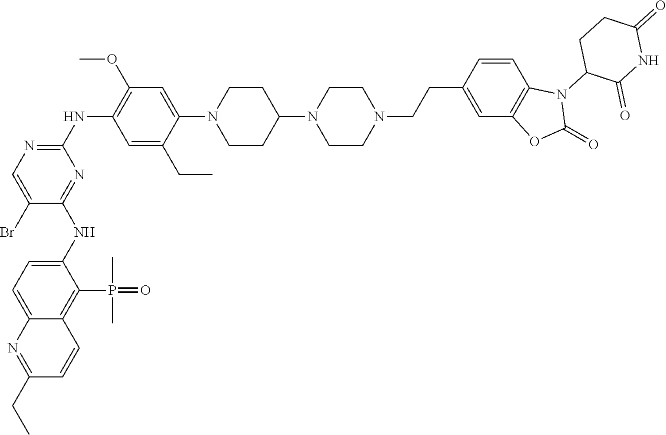 [SMILES: CCc1ccc2c(P(C)(C)=O)c(Nc3nc(Nc4cc(CC)c(N5CCC(N6CCN(CCc7ccc8c(c7)oc(=O)n8C7CCC(=O)NC7=O)CC6)CC5)cc4OC)ncc3Br)ccc2n1]

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (72 mg, 0.10 mmol) and 2-(3-(2,6-dioxopiperidin-3-yl)-2-oxo-2,3-dihydrobenzo[d]oxazol-6-yl)acetaldehyde (35 mg, 0.12 mmol) in DCM (8 mL) was stirred in a flask at room temperature for 10 min. To the mixture was added NaBH₃CN (65 mg, 0.31 mmol) and the reaction was stirred at room temperature for another 0.5 h. Then the mixture was evaporated in vacuum to afford the crude product, which was purified with prep-HPLC chromatography (0.1% FA in water: acetonitrile=90:10˜ 50:50 gradient elution) to give the product (23 mg, 23.3%). ¹H NMR (500 MHz, DMSO) δ 11.77 (s, 1H), 11.21 (s, 1H), 8.58 (d, J=8.9 Hz, 1H), 8.27 (s, 1H), 8.21 (s, 1H), 8.00 (s, 1H), 7.88 (d, J=9.2 Hz, 1H), 7.45 (d, J=8.9 Hz, 1H), 7.36 (s, 1H), 7.33 (s, 1H), 7.18 (d, J=8.1 Hz, 1H), 7.10 (d, J=7.9 Hz, 1H), 6.75 (s, 1H), 5.35 (dd, J=12.9, 5.3 Hz, 1H), 3.76 (s, 3H), 3.31-3.20 (m, 6H), 2.93-2.88 (m, 9H), 2.76-2.56 (m, 8H), 2.31-2.28 (m, 2H), 2.19-2.11 (m, 1H), 1.98-1.95 (m, 7H), 1.61 (s, 2H), 1.32 (t, J=7.6 Hz, 3H), 0.76 (s, 3H). [M+H]⁺=993.7.

### Example 492: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-fluoro-2-oxobenzo[d]oxazol-3(2H)-yl)piperidine-2,6-dione

### Step 1: 6-bromo-5-fluorobenzo[d]oxazol-2(3H)-one

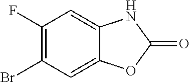 [SMILES: O=c1[nH]c2cc(F)c(Br)cc2o1]

A mixture of 2-amino-5-bromo-4-fluorophenol (3 g, 14.63 mmol), CDI (2.84 g, 17.6 mmol) in THF (50 mL) was stirred at 80° C. for 3 hrs. After cooling to rt, the reaction mixture was concentrated in vacuo. The residue was dissolved in EA (60 mL), which was washed with water (30 mL) and brine (30 mL), dried over Na₂SO₄, filtered and concentrated in vacuum to afford 6-bromo-5-fluorobenzo[d]oxazol-2(3H)-one (3.2 g, 94.1%). [M+H]⁺=231.9.

### Step 2: 3-(2,6-bis(benzyloxy)pyridin-3-yl)-6-bromo-5-fluorobenzo[d]oxazol-2(3H)-one

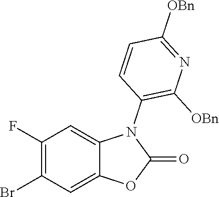 [SMILES: O=c1oc2cc(Br)c(F)cc2n1-c1ccc(OCc2ccccc2)nc1OCc1ccccc1]

To a mixture of 6-bromo-5-fluorobenzo[d]oxazol-2(3H)-one (3 g, 12.9 mmol), 2,6-bis(benzyloxy)-3-(4,4,5,5-tetramethyl-1,3,2-dioxaborolan-2-yl)pyridine (6.47 g, 15.5 mmol) in dioxane (30 mL) was added pyridine (10.2 g, 129 mmol), Cu(OAc)₂ (2.58 g, 12.9 mmol) and 4A molecular sieve (2.5 g). The mixture was stirred at 80° C. under oxygen atmosphere for 3ds. After cooling to r.t, the mixture was filtered and concentrated in vacuo. The residue was purified by silica gel column (PE:EA=8:1) to afford the product (3.3 g, 49.1%). [M+H]⁺=521.1.

### Step 3: 6-(2-(benzyloxy)ethyl)-3-(2,6-bis(benzyloxy)pyridin-3-yl)-5-fluorobenzo[d]oxazol-2(3H)-one

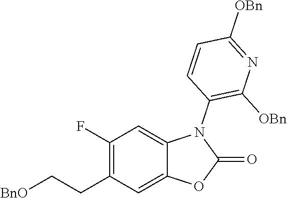 [SMILES: O=c1oc2cc(CCOCc3ccccc3)c(F)cc2n1-c1ccc(OCc2ccccc2)nc1OCc1ccccc1]

To a mixture of 3-(2,6-bis(benzyloxy)pyridin-3-yl)-6-bromo-5-fluorobenzo[d]oxazol-2(3H)-one (1 g, 1.92 mmol), NiI₂ (120 mg, 0.38 mmol), ((2-bromoethoxy)methyl)benzene (619 mg, 2.88 mmol), picolinimidamide hydrochloride (60 mg, 0.38 mmol), NaI (144 mg, 0.96 mmol), and Mn (317 mg, 5.76 mmol) in DMA (20 mL), was added TFA (66 mg, 0.58 mmol) under nitrogen atmosphere. The resulting mixture was stirred at 100° C. for 3 hrs. After cooling to r.t, the reaction was diluted with EA (60 mL) and then washed with brine (20 mL×3), dried over Na₂SO₄, filtered and concentrated in vacuum. The residue was purified by silica gel column (PE:EA=10:1) to afford 6-(2-(benzyloxy)ethyl)-3-(2,6-bis(benzyloxy)pyridin-3-yl)-5-fluorobenzo[d]oxazol-2(3H)-one (330 mg, 29.8%). [M+H]⁺=577.2.

### Step 4: 3-(5-fluoro-6-(2-hydroxyethyl)-2-oxobenzo[d]oxazol-3(2H)-yl)piperidine-2,6-dione

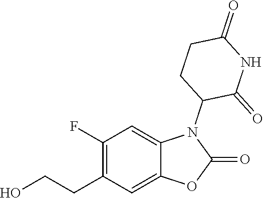 [SMILES: O=C1CCC(n2c(=O)oc3cc(CCO)c(F)cc32)C(=O)N1]

To a mixture of 6-(2-(benzyloxy)ethyl)-3-(2,6-bis(benzyloxy)pyridin-3-yl)-5-fluorobenzo[d]oxazol-2(3H)-one (330 mg, 0.57 mmol) in DMF (8 mL) and EtOH (2 mL) was added Pd/C (150 mg, 10 wt. %, wet). The resulting mixture was stirred at r.t under hydrogen atmosphere for 16 hrs. The reaction mixture was filtered and concentrated in vacuo to afford 3-(5-fluoro-6-(2-hydroxyethyl)-2-oxobenzo[d]oxazol-3(2H)-yl)piperidine-2,6-dione (160 mg, 91%). [M+H]⁺=309.1.

### Step 5: 2-(3-(2,6-dioxopiperidin-3-yl)-5-fluoro-2-oxo-2,3-dihydrobenzo[d]oxazol-6-yl)acetaldehyde

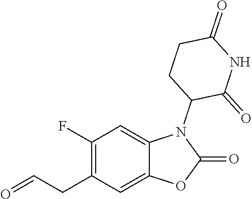 [SMILES: O=CCc1cc2oc(=O)n(C3CCC(=O)NC3=O)c2cc1F]

To a solution of 3-(5-fluoro-6-(2-hydroxyethyl)-2-oxobenzo[d]oxazol-3(2H)-yl)piperidine-2,6-dione (160 mg, 0.52 mmol) in THF (3 mL) and DCM (3 mL) was added DMP (329 mg, 0.78 mmol). After stirring at r. t. for 2hs, the reaction was diluted with water (6 mL), and the layers were separated. The aqueous layer was extracted with DCM (10 mL×3). The combined organic layers were washed with brine (30 mL×3), dried over Na₂SO₄, filtered and concentrated in vacuum. The residue was purified by silica gel column (DCM:CH3OH=15:1) to afford 2-(3-(2,6-dioxopiperidin-3-yl)-5-fluoro-2-oxo-2,3-dihydrobenzo[d]oxazol-6-yl)acetaldehyde (100 mg, 62.5%). [M+H]⁺=307.1.

### Step 6: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-fluoro-2-oxobenzo[d]oxazol-3(2H)-yl)piperidine-2,6-dione

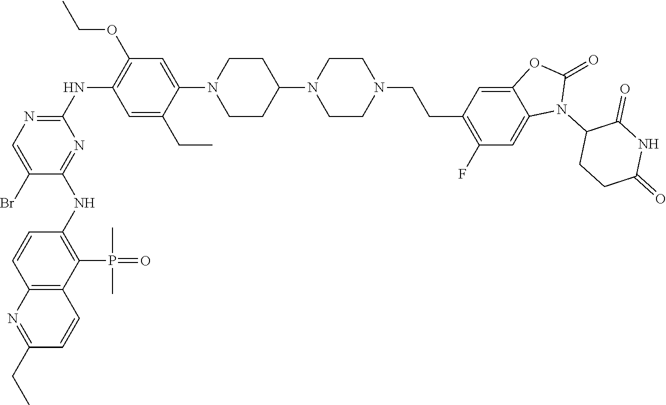 [SMILES: CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2ccc3nc(CC)ccc3c2P(C)(C)=O)n1]

To a solution of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (50 mg, 0.068 mmol, the compound was obtained similar to example 484), 2-(3-(2,6-dioxopiperidin-3-yl)-5-fluoro-2-oxo-2,3-dihydrobenzo[d]oxazol-6-yl)acetaldehyde (25 mg, 0.082 mmol) in DCM (4 mL) was added STAB (28.8 mg, 0.136 mmol). After stirring at r. t. for 2 h, the reaction was diluted with water (6 mL), and the layers were separated. The aqueous layer was extracted with DCM (10 mL×3). The combined organic layers were washed with brine (30 mL×3), dried over Na₂SO₄, filtered and concentrated in vacuum. The residue was purified by prep-HPLC (C-18 column chromatography (0.1% FA in water: acetonitrile=90: 10˜ 60: 40 gradient elution) to afford the product (16.7 mg, 24%). ¹H NMR (500 MHz, DMSO) δ 11.73 (s, 1H), 11.21 (s, 1H), 8.59 (d, J=8.9 Hz, 1H), 8.26-8.24 (m, 1H), 8.23 (s, 1H), 8.00-7.77 (m, 2H), 7.53-7.36 (m, 3H), 7.29-7.27 (m, 1H), 6.65 (s, 1H), 5.36-5.31 (m, 1H), 4.06-3.92 (m, 2H), 2.96-2.91 (m, 4H), 2.89-2.81 (m, 1H), 2.81-2.74 (m, 2H), 2.73-2.70 (m, 1H), 2.65-2.61 (m, 4H), 2.53-2.51 (m, 3H), 2.49-2.42 (m, 4H), 2.27-2.23 (m, 4H), 2.19-2.10 (m, 1H), 2.00 (s, 3H), 1.97 (s, 3H), 1.82 (d, J=12.3 Hz, 2H), 1.54-1.51 (m, 2H), 1.33-1.30 (m, 3H), 1.27-1.24 (m, 4H), 0.75-0.69 (m, 3H). [M+H]⁺=1025.9.

### Example 493: 3-(7-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2-oxobenzo[d]oxazol-3 (2H)-yl)piperidine-2,6-dione

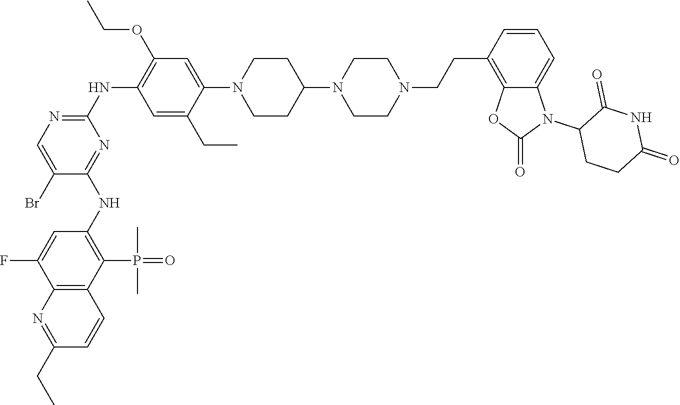 [SMILES: CCOc1cc(N2CCC(N3CCN(CCc4cccc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1]

The title compound (31 mg, 56%) was prepared in a manner similar to that in Example 492. ¹H NMR (500 MHz, DMSO) δ 12.10 (s, 1H), 11.21 (s, 1H), 8.56 (d, J=8.8 Hz, 1H), 8.39-8.10 (m, 2H), 8.04 (s, 1H), 7.54 (d, J=9.0 Hz, 1H), 7.39 (s, 1H), 7.17-7.01 (m, 3H), 6.71 (s, 1H), 5.36 (dd, J=12.8, 5.2 Hz, 1H), 4.01 (q, J=6.9 Hz, 2H), 3.01-2.81 (m, 7H), 2.77-2.52 (m, 13H), 2.38-2.08 (m, 5H), 2.00 (d, J=13.3 Hz, 6H), 1.85-1.83 (m, 2H), 1.57-1.50 (m, 2H), 1.32 (t, J=7.6 Hz, 3H), 1.26 (t, J=6.9 Hz, 3H), 0.75 (s, 3H). [M+H]⁺=1025.8.

### Example 496: 3-(7-(3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)propyl)-5-fluoro-2-oxobenzo[d]oxazol-3(2H)-yl)piperidine-2,6-dione

### Step 1: 7-(3-(benzyloxy)propyl)-3-(2,6-bis(benzyloxy)pyridin-3-yl)-5-fluorobenzo[d]oxazol-2(3H)-one

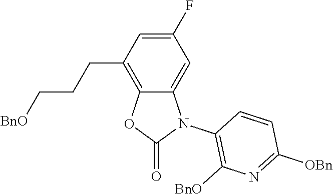 [SMILES: O=c1oc2c(CCCOCc3ccccc3)cc(F)cc2n1-c1ccc(OCc2ccccc2)nc1OCc1ccccc1]

The title compound (180 mg, 32.6%) was prepared in a manner similar to that in Example 492 step 3 from 3-(2,6-bis(benzyloxy)pyridin-3-yl)-7-bromo-5-fluorobenzo[d]oxazol-2(3H)-one and ((3-bromopropoxy)methyl)benzene. [M+H]⁺=591.2.

### Step 2: 3-(5-fluoro-7-(3-hydroxypropyl)-2-oxobenzo[d]oxazol-3(2H)-yl)piperidine-2,6-dione

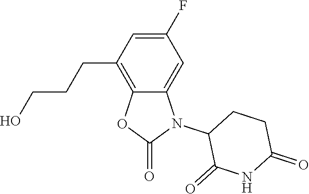 [SMILES: O=C1CCC(n2c(=O)oc3c(CCCO)cc(F)cc32)C(=O)N1]

The title compound (90 mg, 78.9%) was prepared in a manner similar to that in Example 492 step 4 from 7-(3-(benzyloxy)propyl)-3-(2,6-bis(benzyloxy)pyridin-3-yl)-5-fluorobenzo[d]oxazol-2(3H)-one and Pd/C (10 wt. %, wet). [M+H]⁺=323.1.

### Step 3: 3-(3-(2,6-dioxopiperidin-3-yl)-5-fluoro-2-oxo-2,3-dihydrobenzo[d]oxazol-7-yl)propanal

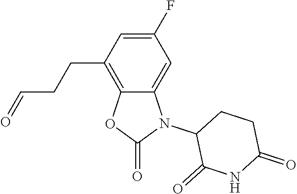 [SMILES: O=CCCc1cc(F)cc2c1oc(=O)n2C1CCC(=O)NC1=O]

The title compound (60 mg, 87.6%) was prepared in a manner similar to that in Example 492 step 5 from 3-(5-fluoro-7-(3-hydroxypropyl)-2-oxobenzo[d]oxazol-3(2H)-yl)piperidine-2,6-dione and HCOOH. [M+H]⁺=321.1.

### Step 4: 3-(7-(3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)propyl)-5-fluoro-2-oxobenzo[d]oxazol-3(2H)-yl)piperidine-2,6-dione

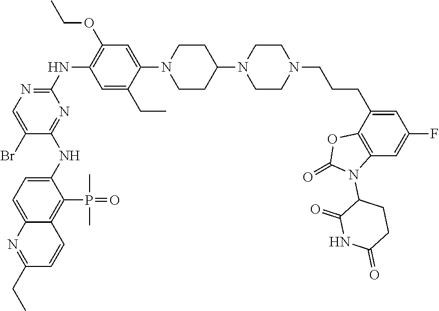 [SMILES: CCOc1cc(N2CCC(N3CCN(CCCc4cc(F)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

A mixture of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (75 mg, 0.10 mmol) and 3-(3-(2,6-dioxopiperidin-3-yl)-5-fluoro-2-oxo-2,3-dihydrobenzo[d]oxazol-7-yl)propanal (38 mg, 0.12 mmol) in DCM (8 mL) was stirred in a flask at room temperature for 10 min. To the mixture was added sodium triacetoxyborohydride (65 mg, 0.31 mmol) and the reaction was stirred at room temperature for another 0.5 h. Then the mixture was evaporated in vacuum to afford the crude product, which was purified with prep-HPLC chromatography (0.1% FA in water: acetonitrile=90:10˜50:50 gradient elution) to give the product (30 mg, 28.4%). ¹H NMR (500 MHz, DMSO) δ 11.73 (s, 1H), 11.21 (s, 1H), 8.59 (d, J=8.8 Hz, 1H), 8.25 (s, 1H), 8.22 (s, 1H), 7.91 (s, 1H), 7.87 (d, J=9.6 Hz, 1H), 7.45 (d, J=8.9 Hz, 1H), 7.38 (s, 1H), 7.16 (d, J=6.2 Hz, 1H), 6.92 (d, J=8.3 Hz, 1H), 6.71 (s, 1H), 5.35 (dd, J=13.0, 5.5 Hz, 1H), 4.00 (q, J=7.0 Hz, 2H), 3.29 (s, 1H), 2.97-2.82 (m, 5H), 2.76-2.54 (m, 10H), 2.42-2.23 (m, 8H), 2.18-2.13 (m, 1H), 1.98 (d, J=13.3 Hz, 6H), 1.82-1.77 (m, 4H), 1.54-1.51 (m, 2H), 1.32 (s, 3H), 1.25 (t, J=6.9 Hz, 3H), 0.71 (s, 3H). [M+H]⁺=1039.7

### Example 497: 3-(7-(3-((4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)methyl)azetidin-1-yl)-2-oxobenzo[d]oxazol-3 (2H)-ylpiperidine-2,6-dione

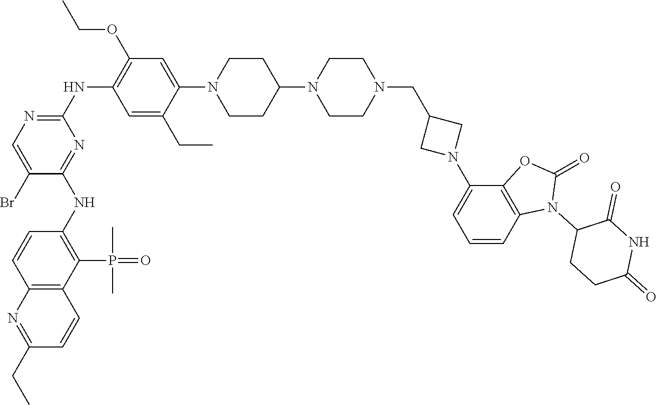 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

A mixture of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (75 mg, 0.10 mmol) and 1-(3-(2,6-dioxopiperidin-3-yl)-2-oxo-2,3-dihydrobenzo[d]oxazol-7-yl)azetidine-3-carbaldehyde (40 mg, 0.12 mmol) in DCM (8 mL) was stirred in a flask at room temperature for 10 min. To the mixture was added sodium triacetoxyborohydride (65 mg, 0.31 mmol) and the reaction was stirred at room temperature for another 0.5 h. Then the mixture was evaporated in vacuum to afford the crude product, which was purified with prep-HPLC chromatography (0.1% FA in water: acetonitrile=90:10˜ 50:50 gradient elution) to give the product (33 mg, 31.2%). ¹H NMR (500 MHz, DMSO) δ 11.72 (s, 1H), 11.19 (s, 1H), 8.60 (d, J=8.9 Hz, 1H), 8.25 (s, 1H), 8.22 (d, J=4.7 Hz, 1H), 7.91 (s, 1H), 7.88 (d, J=9.3 Hz, 1H), 7.45 (d, J=8.9 Hz, 1H), 7.39 (s, 1H), 7.01 (t, J=8.1 Hz, 1H), 6.71 (s, 1H), 6.57 (d, J=7.9 Hz, 1H), 6.25 (d, J=8.2 Hz, 1H), 5.29 (dd, J=12.9, 5.4 Hz, 1H), 4.11 (t, J=7.5 Hz, 2H), 4.00 (q, J=7.0 Hz, 2H), 3.65 (t, J=6.6 Hz, 2H), 3.26-3.20 (m, 2H), 2.98-2.83 (m, 6H), 2.70-2.52 (m, 9H), 2.44-2.38 (m, 3H), 2.32-2.20 (m, 3H), 2.16-2.08 (m, 1H), 1.98 (d, J=13.3 Hz, 6H), 1.83 (d, J=11.5 Hz, 2H), 1.54-1.51 (m, 2H), 1.32 (t, J=7.6 Hz, 3H), 1.25 (t, J=6.9 Hz, 3H), 0.71 (s, 3H). [M+H]⁺=1048.7

### Example 498: 3-(6-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-5-fluoro-2-oxobenzo[d]oxazol-3(2H)-yl)piperidine-2,6-dione

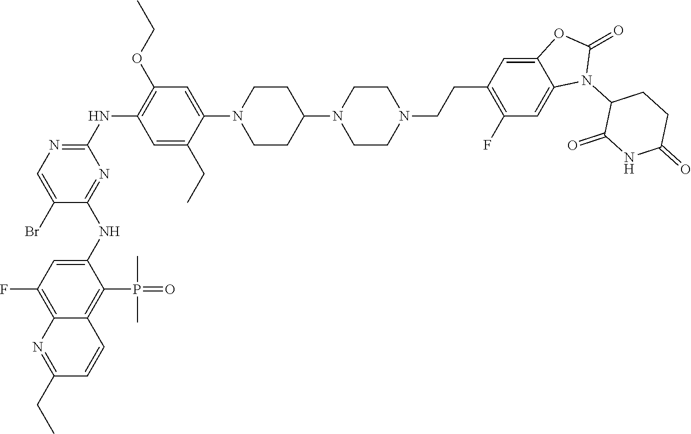 [SMILES: CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2cc(F)c3nc(CC)ccc3c2P(C)(C)=O)n1]

The title compound was prepared in a manner similar to that in Example 492.

¹H NMR (500 MHz, DMSO) δ 12.04 (s, 1H), 11.14 (s, 1H), 8.50 (d, J=8.5 Hz, 1H), 8.09-8.34 (m, 2H), 7.97 (s, 1H), 7.47 (d, J=9.0 Hz, 1H), 7.34 (d, J=6.1 Hz, 2H), 7.22 (d, J=9.5 Hz, 1H), 6.64 (s, 1H), 5.28 (dd, J=13.0, 5.3 Hz, 1H), 3.94 (q, J=6.9 Hz, 2H), 2.75-2.94 (m, 5H), 2.46-2.74 (m, 11H), 2.15-2.40 (m, 8H), 2.05-2.10 (m, 1H), 1.93 (d, J=13.3 Hz, 6H), 1.74-1.76 (m, 2H), 1.38-1.56 (m, 2H), 1.25 (t, J=7.6 Hz, 3H), 1.19 (t, J=6.9 Hz, 3H), 0.68 (s, 3H). [M+H]⁺=1043.7.

### Example 499: 3-(6-(2-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

### Step 1: (6-((5-bromo-2-((5-ethyl-2-methoxy-4-(4-(piperazin-1-yl)piperidin-1-yl)phenyl)amino)pyrimidin-4-yl)amino)-2-ethylquinazolin-5-yl)dimethylphosphine oxide

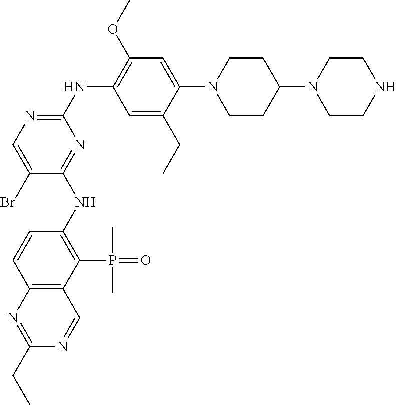 [SMILES: CCc1ncc2c(P(C)(C)=O)c(Nc3nc(Nc4cc(CC)c(N5CCC(N6CCNCC6)CC5)cc4OC)ncc3Br)ccc2n1]

To a solution of (6-((5-bromo-2-chloropyrimidin-4-yl)amino)-2-ethylquinazolin-5-yl)dimethylphosphine oxide (300 mg, 0.68 mmol) and tert-butyl 4-(1-(4-amino-2-ethyl-5-methoxyphenyl)piperidin-4-yl)piperazine-1-carboxylate (342 mg, 0.82 mmol) in n-BuOH (10 mL) was added Ts-OH (350 mg, 2.04 mmol) at room temperature. The resulting mixture was stirred at 100° C. overnight. The reaction was concentrated and basified with 0.5N NaOH (10 mL), then extracted with DCM (3×10 mL). The combined organic layers were dried over anhydrous Na₂SO₄, filtered and evaporated in vacuum to afford the crude residue, which was purified by silica gel column chromatography (DCM: MeOH=100: 0-5: 1 gradient elution) to give the desired product (230 mg, 46%). [M+H]⁺=722.2.

### Step 2: 3-(6-(2-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

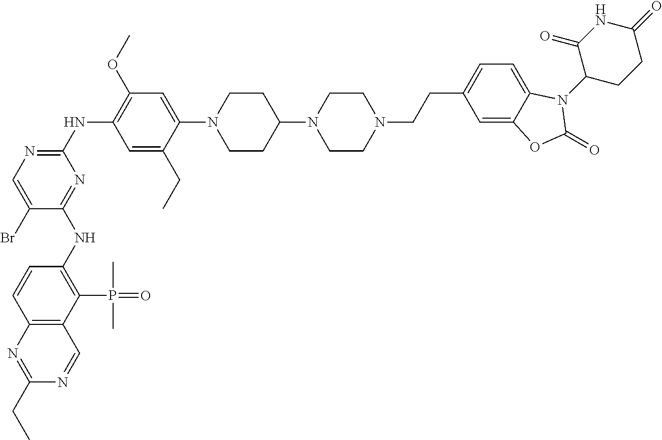 [SMILES: CCc1ncc2c(P(C)(C)=O)c(Nc3nc(Nc4cc(CC)c(N5CCC(N6CCN(CCc7ccc8c(c7)oc(=O)n8C7CCC(=O)NC7=O)CC6)CC5)cc4OC)ncc3Br)ccc2n1]

To a solution of (6-((5-bromo-2-((5-ethyl-2-methoxy-4-(4-(piperazin-1-yl)piperidin-1-yl)phenyl)amino)pyrimidin-4-yl)amino)-2-ethylquinazolin-5-yl)dimethylphosphine oxide (60 mg, 0.083 mmol) and 2-(3-(2,6-dioxopiperidin-3-yl)-2-oxo-2,3-dihydrobenzo[d]oxazol-6-yl)acetaldehyde (36 mg, 0.125 mmol) in DCM (5 mL) was added sodium triacetoxyborohydride (60 mg, 0.28 mmol) at room temperature. The resulting mixture was stirred at room temperature for 1 hour. The reaction was quenched with water (10 mL) and extracted with DCM (2×10 mL). The combined organic layers were dried over anhydrous Na₂SO₄, filtered and evaporated in vacuum to afford the crude residue, which was purified by silica gel column chromatography (DCM: MeOH=100: 0-10: 1 gradient elution) to give an impure product, which was further purified with prep-HPLC to give the desired product (14.62 mg, 17.8%). ¹H NMR (500 MHz, DMSO) δ 11.60 (s, 1H), 11.20 (s, 1H), 9.86 (s, 1H), 8.45 (s, 1H), 8.23 (s, 1H), 8.05 (s, 1H), 7.85 (d, J=9.2 Hz, 1H), 7.29 (d, J=13.2 Hz, 2H), 7.16 (d, J=8.0 Hz, 1H), 7.08 (d, J=8.1 Hz, 1H), 6.74 (s, 1H), 5.35 (dd, J=12.6, 5.1 Hz, 1H), 3.75 (s, 3H), 3.05 (q, J=7.5 Hz, 2H), 2.96-2.86 (m, 3H), 2.77 (t, J=7.3 Hz, 2H), 2.71-2.63 (m, 5H), 2.57-2.51 (s, 6H), 2.47-2.43 (m, 2H), 2.26 (s, 3H), 2.17-2.13 (m, 1H), 2.03 (d, J=13.4 Hz, 7H), 1.84 (d, J=11.5 Hz, 2H), 1.54 (d, J=10.2 Hz, 2H), 1.38 (t, J=7.5 Hz, 3H), 0.75 (s, 3H); [M+H]⁺=994.7.

### Example 500: 3-(6-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

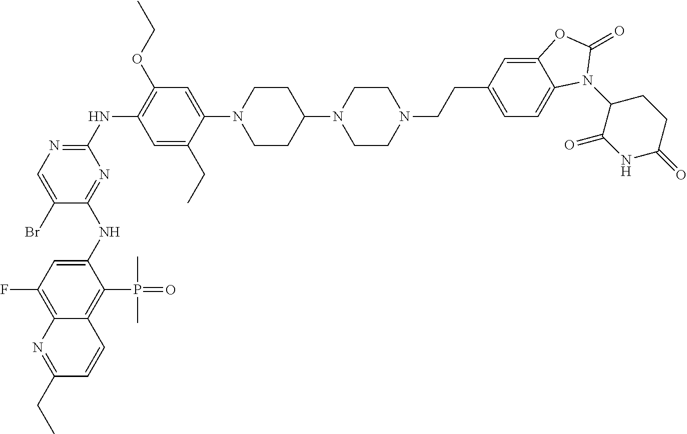 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2cc(F)c3nc(CC)ccc3c2P(C)(C)=O)n1]

The title compound was prepared in a procedure similar to that in Example 492. ¹H NMR (500 MHz, DMSO) δ 12.09 (s, 1H), 11.21 (s, 1H), 8.57 (d, J=8.3 Hz, 1H), 8.17-8.38 (m, 2H), 8.04 (s, 1H), 7.54 (d, J=9.0 Hz, 1H), 7.39 (s, 1H), 7.30 (s, 1H), 7.16 (d, J=8.1 Hz, 1H), 7.08 (d, J=8.0 Hz, 1H), 6.71 (s, 1H), 5.35 (dd, J=12.9, 5.2 Hz, 1H), 4.01 (q, J=6.9 Hz, 2H), 2.88-2.98 (m, 6H), 2.51-2.80 (m, 11H), 2.21-2.48 (m, 7H), 2.09-2.19 (m, 1H), 2.00 (d, J=13.3 Hz, 6H), 1.82-1.84 (m, 2H), 1.49-1.56 (m, 2H), 1.32 (t, J=7.6 Hz, 3H), 1.26 (t, J=6.9 Hz, 3H), 0.74 (s, 3H). [M+H]⁺=1025.6.

### Example 501: 3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-3,3-dimethyl-2-oxoindolin-1-yl)piperidine-2,6-dione

### Step 1: 6-bromo-2-ethylphthalazin-1(2H)-one

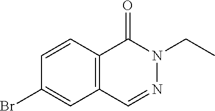

To a stirred solution of 6-bromophthalazin-1(2H)-one (10 g, 44.6 mmol) and Cs₂CO₃ (29.1 g, 89.2 mmol) in DMF (200 mL) was added iodoethane (10.4 g, 66.9 mmol). The resulting mixture was stirred at rt for 16 hours. The mixture was filtrated and the filtrate was concentrated under vacuum to afford the crude product, which was purified with silica gel column chromatography (DCM: MeOH=100:0˜ 100:10 gradient elution) to give the title product (10 g, 88.5%). [M+H]⁺=253.2.

### Step 2: 6-((diphenylmethylene)amino)-2-ethylphthalazin-1(2H)-one

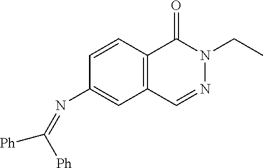 [SMILES: CCn1ncc2cc(N=C(c3ccccc3)c3ccccc3)ccc2c1=O]

To a stirred solution of 6-bromo-2-ethylphthalazin-1(2H)-one (10 g, 39.7 mmol), Cs₂CO₃ (25.9 g, 79.4 mmol), Pd₂(dba)₃ (3.6 g, 4.0 mmol), and BINAP(4.9 g, 7.9 mmol) in dioxane (200 mL) was added diphenylmethanimine (14.4 g, 79.4 mmol). The resulting mixture was stirred at 100° C. for 16 hours under nitrogen atmosphere. The mixture was filtrated and the filtrate was concentrated under vacuum to afford the crude product, which was purified with silica gel column chromatography (PE: EA=100:0˜ 1:1 gradient elution) to give the title product (12 g, 85.6%). [M+H]⁺=354.4.

### Step 3: 6-amino-2-ethylphthalazin-1(2H)-one

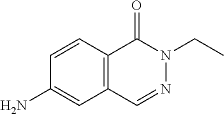

A solution of 6-((diphenylmethylene)amino)-2-ethylphthalazin-1(2H)-one (12 g, 34 mmol) in 4N HCl in 1,4-dioxane (150 mL) was stirred in a round bottom flask at room temperature overnight. The mixture was concentrated under reduced pressure to afford the crude product (6 g, 93%), which was used for next step without further purification. [M+H]⁺=190.0.

### Step 4: 6-amino-2-ethyl-5-iodophthalazin-1(2H)-one

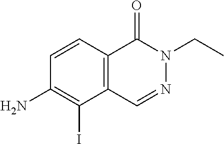

To a stirred solution of 6-amino-2-ethylphthalazin-1(2H)-one (6 g, 31.6 mmol) in AcOH (100 mL) was added ICl (7.7 g, 47.7 mmol) at 20° C. Then the mixture was stirred at 20° C. for 1 hour.

Then the mixture was adjusted to pH=8 with sat. aq. NaHCO₃ and extracted with DCM (3×100 mL). The combined organic layers were washed with brine (3×80 mL), dried over Na₂SO₄, filtered and concentrated in vacuum to afford the crude product, which was purified with silica gel column chromatography (DCM: MeOH=100:0˜ 10:1 gradient elution) to give the title product (8 g, 80%). [M+H]⁺=316.0.

### Step 5: 6-amino-5-(dimethylphosphoryl)-2-ethylphthalazin-1(2H)-one

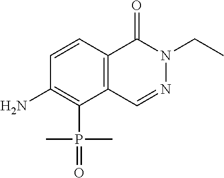 [SMILES: CCn1ncc2c(P(C)(C)=O)c(N)ccc2c1=O]

To a solution of 6-amino-2-ethyl-5-iodophthalazin-1(2H)-one (8 g, 25.4 mmol) and dimethylphosphine oxide (3.9 g, 50.8 mmol) in dioxane (120 mL) was added K₃PO₄ (10.8 g, 50.8 mmol) at 20° C. Pd(OAc)₂ (569 mg, 2.54 mmol) and Xantphos (2.8 g, 5.1 mmol) were added to the mixture at 20° C. The flask was evacuated and backfilled with nitrogen three times. Then the mixture was stirred at 100° C. overnight. The mixture was filtered and concentrated in vacuum. The residue was purified by silica gel column chromatography (DCM: MeOH=100: 0˜10: 1 gradient elution) to give the desired product (6 g, 89%). [M+H]⁺=266.0.

### Step 6: 6-((5-bromo-2-chloropyrimidin-4-yl)amino)-5-(dimethylphosphoryl)-2-ethylphthalazin-1(2H)-one

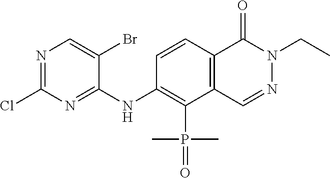 [SMILES: CCn1ncc2c(P(C)(C)=O)c(Nc3nc(Cl)ncc3Br)ccc2c1=O]

To a solution of 6-amino-5-(dimethylphosphoryl)-2-ethylphthalazin-1(2H)-one (6 g, 22.6 mmol) and 5-bromo-2,4-dichloropyrimidine (10.2 g, 45.2 mmol) in THF (100 mL) was added LiHMDS (1 M in THF, 45.2 mL, 45.2 mmol) at room temperature. The resulting mixture was stirred at rt for 1 hour. The reaction was concentrated to afford the crude residue, which was purified by silica gel column chromatography (DCM: MeOH=100: 0˜15: 1 gradient elution) to give the desired product (8 g, 78%). [M+H]⁺=456.2.

### Step 7: 6-((5-bromo-2-((2-ethoxy-5-ethyl-4-(4-(piperazin-1-yl)piperidin-1-yl)phenyl)amino)pyrimidin-4-yl)amino)-5-(dimethylphosphoryl)-2-ethylphthalazin-1(2H)-one

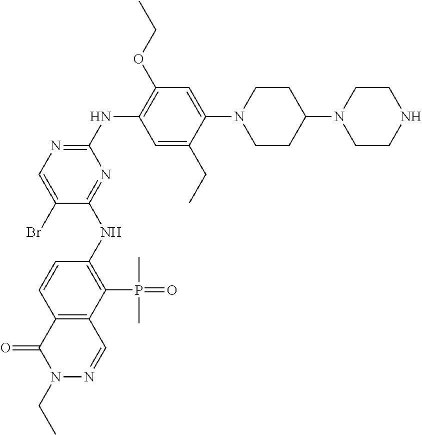 [SMILES: CCOc1cc(N2CCC(N3CCNCC3)CC2)c(CC)cc1Nc1ncc(Br)c(Nc2ccc3c(=O)n(CC)ncc3c2P(C)(C)=O)n1]

To a solution of 6-((5-bromo-2-chloropyrimidin-4-yl)amino)-5-(dimethylphosphoryl)-2-ethylphthalazin-1(2H)-one (3 g, 6.6 mmol) and tert-butyl 4-(1-(4-amino-5-ethoxy-2-ethylphenyl)piperidin-4-yl)piperazine-1-carboxylate (2.9 g, 6.6 mmol) in n-BuOH (100 mL) was added Ts-OH (3.5 g, 19.8 mmol) at room temperature. The resulting mixture was stirred at 100° C. overnight. The reaction was concentrated and basified with 0.5N NaOH (50 mL), then extracted with DCM (3×80 mL). The combined organic layers were dried over anhydrous Na₂SO₄, filtered and evaporated in vacuum to afford the crude residue, which was purified by silica gel column chromatography (DCM: MeOH=100: 0˜5: 1 gradient elution) to give the desired product (3 g, 61%). [M+H]⁺=752.2.

### Step 8: 3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-3,3-dimethyl-2-oxoindolin-1-yl)piperidine-2,6-dione

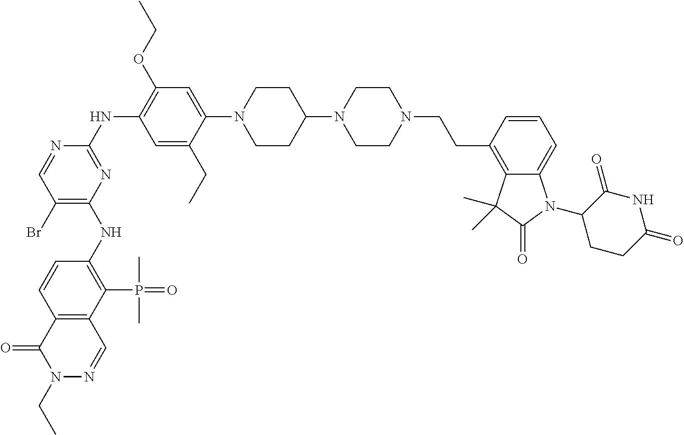 [SMILES: CCOc1cc(N2CCC(N3CCN(CCc4cccc5c4C(C)(C)C(=O)N5C4CCC(=O)NC4=O)CC3)CC2)c(CC)cc1Nc1ncc(Br)c(Nc2ccc3c(=O)n(CC)ncc3c2P(C)(C)=O)n1]

To a solution of 6-((5-bromo-2-((2-ethoxy-5-ethyl-4-(4-(piperazin-1-yl)piperidin-1-yl)phenyl)amino)pyrimidin-4-yl)amino)-5-(dimethylphosphoryl)-2-ethylphthalazin-1(2H)-one (50 mg, 0.06 mmol) and 2-(1-(2,6-dioxopiperidin-3-yl)-3,3-dimethyl-2-oxoindolin-4-yl)acetaldehyde (23 mg, 0.07 mmol, the compound was obtained by the way similar to example 413) in DCM (10 mL) was added NaBH(OAc)₃ (30 mg, 0.14 mmol) at room temperature. The resulting mixture was stirred at room temperature for 1 hour. The reaction was quenched with water (10 mL) and the layers were separated. The aqueous layer was extracted with DCM (3×10 mL). The combined organic layers were dried over anhydrous Na₂SO₄, filtered and evaporated in vacuum to afford the crude residue, which was purified by silica gel column chromatography (DCM: MeOH=100: 0˜10: 1 gradient elution) to give the product (20 mg, 32%). ¹H NMR (500 MHz, DMSO) δ 12.11 (s, 1H), 11.06 (s, 1H), 8.64 (s, 1H), 8.53 (s, 1H), 8.27 (s, 1H), 8.19 (d, J=9.3 Hz, 1H), 8.10 (s, 1H), 7.33 (s, 1H), 7.18 (t, J=7.8 Hz, 1H), 6.92 (d, J=7.7 Hz, 1H), 6.83 (s, 1H), 6.74 (s, 1H), 5.21 (s, 1H), 4.17 (q, J=7.2 Hz, 2H), 4.00 (q, J=6.9 Hz, 2H), 2.98 (d, J=10.5 Hz, 2H), 2.84 (s, 4H), 2.72-2.53 (m, 11H), 2.40 (d, J=7.4 Hz, 4H), 2.04 (d, J=13.4 Hz, 6H), 1.95-1.90 (m, 4H), 1.58 (s, 2H), 1.38 (d, J=4.1 Hz, 6H), 1.32 (t, J=7.2 Hz, 3H), 1.25 (t, J=6.8 Hz, 3H), 0.89 (s, 3H). [M+H]⁺=1050.4.

### Example 502: (R)-3-(4-(2-(4-(1-(4-((5-bromo-4-((5-(dimethylphosphoryl)-2-methyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

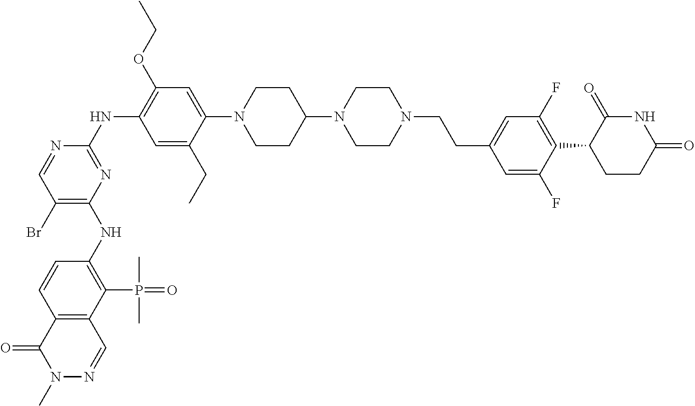 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3c(=O)n(C)ncc3c2P(C)(C)=O)n1]

The title compound (497 mg, 60%) was prepared in a manner similar to that in Example 484. ¹H NMR (500 MHz, DMSO) δ 12.11 (s, 1H), 10.85 (s, 1H), 8.64 (s, 1H), 8.52 (s, 1H), 8.26 (s, 1H), 8.18 (d, J=8.9 Hz, 1H), 8.09 (s, 1H), 7.34 (s, 1H), 6.73 (s, 1H), 6.10 (d, J=11.1 Hz, 2H), 4.17 (d, J=7.1 Hz, 2H), 4.00 (d, J=6.9 Hz, 3H), 3.93 (s, 2H), 3.47 (s, 3H), 3.00-2.87 (m, 3H), 2.81-2.73 (m, 1H), 2.64 (t, J=11.1 Hz, 3H), 2.55 (d, J=6.8 Hz, 4H), 2.37-2.32 (m, 6H), 2.03-2.01 (m, 7H), 1.97-1.91 (m, 1H), 1.83 (d, J=11.0 Hz, 2H), 1.55 (dd, J=20.3, 11.3 Hz, 2H), 1.31 (t, J=7.1 Hz, 3H), 1.25 (t, J=6.9 Hz, 3H). [M+H]⁺=989.4.

### Example 503: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-fluoro-3-methyl-2-oxo-2,3-dihydro-1H-benzo[d]imidazol-1-yl)piperidine-2,6-dione

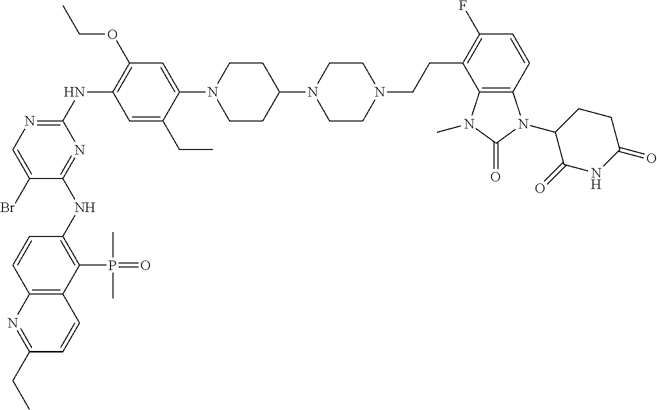 [SMILES: CCOc1cc(N2CCC(N3CCN(CCc4c(F)ccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound (40 mg, 32.2%) was prepared in a manner similar to that in Example 318. ¹H NMR (500 MHz, DMSO) δ 11.69 (s, 1H), 11.11 (s, 1H), 8.63 (d, J=8.7 Hz, 1H), 8.26-8.21 (m, 2H), 7.89 (d, J=12.0 Hz, 2H), 7.46 (d, J=8.9 Hz, 1H), 7.42 (s, 1H), 7.02 (s, 1H), 6.91 (t, J=9.5 Hz, 1H), 6.72 (s, 1H), 5.38 (dd, J=12.7, 5.5 Hz, 1H), 4.01 (d, J=7.0 Hz, 2H), 3.60 (s, 3H), 3.32-3.24 (m, 7H), 3.10 (s, 3H), 2.98-2.83 (m, 6H), 2.67-2.64 (m, 7H), 2.27-2.24 (m, 2H), 2.01-1.97 (m, 8H), 1.60-1.57 (m, 2H), 1.33 (t, J=7.6 Hz, 3H), 1.27 (t, J=6.9 Hz, 3H), 0.70 (s, 3H). [M+H]⁺=1038.7.

### Example 504: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3-ethyl-2-oxo-2,3-dihydro-1H-benzo[d]imidazol-1-yl)piperidine-2,6-dione

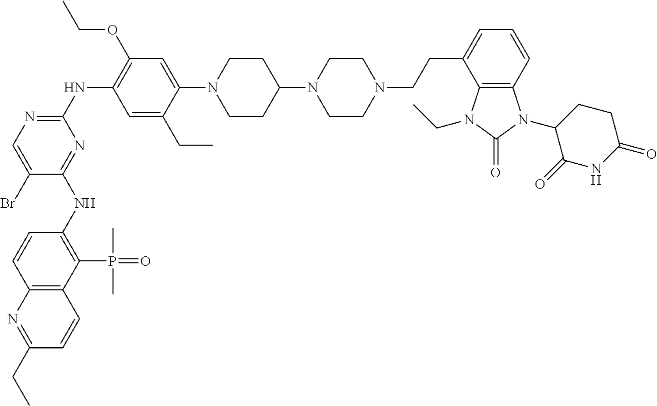 [SMILES: CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound (24 mg, 28.1%) was prepared in a manner similar to that in Example 318. ¹H NMR (500 MHz, DMSO) δ 11.73 (s, 1H), 11.11 (s, 1H), 8.60 (d, J=9.0 Hz, 1H), 8.24 (s, 1H), 8.22 (s, 1H), 7.93 (s, 1H), 7.88 (d, J=9.3 Hz, 1H), 7.45 (d, J=8.9 Hz, 1H), 7.38 (s, 1H), 6.99 (d, J=5.3 Hz, 2H), 6.95-6.88 (m, 1H), 6.71 (s, 1H), 5.37 (dd, J=12.9, 5.2 Hz, 1H), 4.03-4.00 (m, 4H), 3.02-2.87 (m, 8H), 2.59-2.55 (m, 13H), 2.29-2.26 (m, 3H), 2.03-2.00 (m, 1H), 1.98 (d, J=13.3 Hz, 6H), 1.83 (d, J=10.8 Hz, 2H), 1.55-1.51 (m, 2H), 1.32 (t, J=7.6 Hz, 3H), 1.27-1.23 (m, 6H), 0.71 (s, 3H). [M+H]⁺=1034.7

### Example 505: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-4-fluoro-2-oxobenzo[d]oxazol-3(2H)-yl)piperidine-2,6-dione

### Step 1: 2-amino-6-bromo-3-fluorophenol

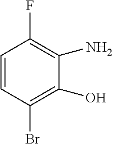

To a solution of 6-bromo-3-fluoro-2-nitrophenol (5 g, 21.3 mmol) in MeOH (100 mL) was added Raney Ni (5 g) at room temperature under hydrogen atmosphere. The resulting mixture was stirred at rt for 2 hours. The reaction was filtered and concentrated under reduced pressure to afford the desired product (4 g, 92%). [M+H]⁺=206.2.

### Step 2: 7-bromo-4-fluorobenzo[d]oxazol-2(3H)-one

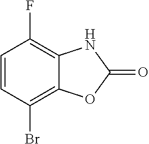 [SMILES: O=c1[nH]c2c(F)ccc(Br)c2o1]

To a solution of 2-amino-6-bromo-3-fluorophenol (4 g, 19.5 mmol) in THF (100 mL) was added CDI (4 g, 23.4 mmol) at room temperature. The resulting mixture was stirred at rt for 2 hours. The reaction was diluted with water (100 mL) and extracted with EA (3×30 mL). The combined organic layers were dried over anhydrous Na₂SO₄, filtered and evaporated in vacuum to afford the crude residue, which was purified by silica gel column chromatography(PE/EA=100:0˜1:1 gradient elution) to give the desired product (3.5 g, 77%). [M+H]⁺=232.3.

### Step 3: 3-(7-bromo-4-fluoro-2-oxobenzo[d]oxazol-3(2H)-yl)piperidine-2,6-dione

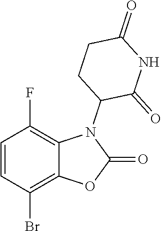 [SMILES: O=C1CCC(n2c(=O)oc3c(Br)ccc(F)c32)C(=O)N1]

To a solution of 7-bromo-4-fluorobenzo[d]oxazol-2(3H)-one (3.5 g, 15.2 mmol) and 3-bromopiperidine-2,6-dione (5.8 g, 30.4 mmol) in DMF (50 mL) was added Cs₂CO₃ (9.9 g, 30.4 mmol) at room temperature. The resulting mixture was stirred at 60° C. overnight. The mixture was filtered, concentrated under reduced pressure and diluted with water (100 mL). The aqueous layer was extracted with DCM (3×30 mL). The combined organic layers were dried over anhydrous Na₂SO₄, filtered and evaporated in vacuum to afford the crude residue, which was mashed with MeOH to give the desired product (1.5 g, 29%). [M+H]⁺=343.2.

### Step 4: (E)-3-(7-(2-ethoxyvinyl)-4-fluoro-2-oxobenzo[d]oxazol-3(2H)-yl)piperidine-2,6-dione

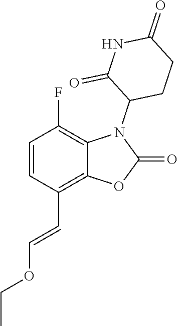 [SMILES: CCO/C=C/c1ccc(F)c2c1oc(=O)n2C1CCC(=O)NC1=O]

To a solution of 3-(7-bromo-4-fluoro-2-oxobenzo[d]oxazol-3(2H)-yl)piperidine-2,6-dione (1.5 g, 4.4 mmol) and (E)-2-(2-ethoxyvinyl)-4,4,5,5-tetramethyl-1,3,2-dioxaborolane (1.7 g, 8.8 mmol) in DMF (30 mL) was added Pd(dtbpf)Cl₂ (286 mg, 0.44 mmol) and CsF (1.3 g, 8.8 mmol) at 20° C. The flask was evacuated and backfilled with nitrogen three times. Then the mixture was stirred at 100° C. overnight. The mixture was filtered and concentrated in vacuum. The residue was purified by silica gel column chromatography (PE:EA=100: 0-0: 100 gradient elution) to give the desired product (1.0 g, 68%). [M+H]⁺=335.4.

### Step 5: 2-(3-(2,6-dioxopiperidin-3-yl)-4-fluoro-2-oxo-2,3-dihydrobenzo[d]oxazol-7-yl)acetaldehyde

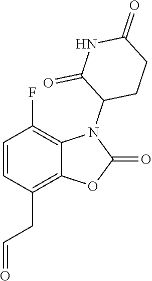 [SMILES: O=CCc1ccc(F)c2c1oc(=O)n2C1CCC(=O)NC1=O]

A solution of (E)-3-(7-(2-ethoxyvinyl)-4-fluoro-2-oxobenzo[d]oxazol-3(2H)-yl)piperidine-2,6-dione (1 g, 3.0 mmol) in FA (20 mL) was stirred at rt for 4 hours. The mixture was concentrated under reduced pressure to afford the desired product (600 mg, 65%). [M+H]⁺=307.1.

### Step 6: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-4-fluoro-2-oxobenzo[d]oxazol-3(2H)-yl)piperidine-2,6-dione

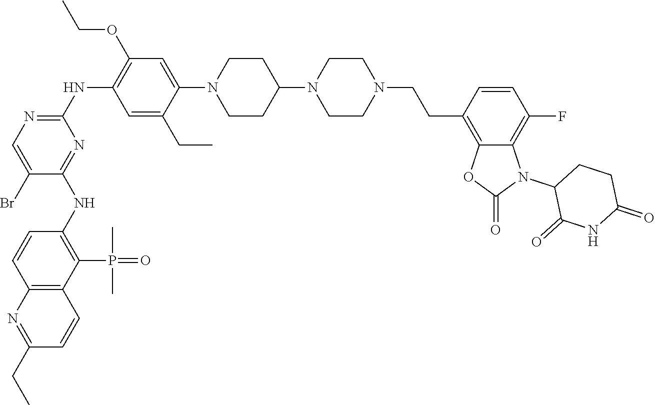 [SMILES: CCOc1cc(N2CCC(N3CCN(CCc4ccc(F)c5c4oc(=O)n5C4CCC(=O)NC4=O)CC3)CC2)c(CC)cc1Nc1ncc(Br)c(Nc2ccc3nc(CC)ccc3c2P(C)(C)=O)n1]

To a solution of (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide (50 mg, 0.07 mmol) and 2-(3-(2,6-dioxopiperidin-3-yl)-4-fluoro-2-oxo-2,3-dihydrobenzo[d]oxazol-7-yl)acetaldehyde (26 mg, 0.08 mmol) in DCM (10 mL) was added NaBH(OAc)₃ (30 mg, 0.14 mmol) at room temperature. The resulting mixture was stirred at room temperature for 1 hour. The reaction was quenched with water (10 mL) and the layers were separated. The aqueous layer was extracted with DCM (3×10 mL). The combined organic layers were dried over anhydrous Na₂SO₄, filtered and evaporated in vacuum to afford the crude residue, which was purified by silica gel column chromatography (DCM: MeOH=100: 0-10: 1 gradient elution) to give an impure product, which was further purified with prep-HPLC to give the desired product (12 mg, 17%). ¹H NMR (500 MHz, DMSO) δ 11.73 (s, 1H), 11.24 (s, 1H), 8.60 (d, J=8.8 Hz, 1H), 8.23 (d, J=9.9 Hz, 2H), 7.95-7.83 (m, 2H), 7.45 (d, J=8.9 Hz, 1H), 7.38 (s, 1H), 7.15-7.07 (m, 2H), 6.71 (s, 1H), 5.44 (s, 1H), 3.99 (t, J=7.0 Hz, 2H), 2.93 (dd, J=15.0, 7.5 Hz, 6H), 2.84 (t, J=7.5 Hz, 2H), 2.68-2.53 (m, 11H), 2.32-2.14 (m, 6H), 1.98 (d, J=13.3 Hz, 6H), 1.82 (d, J=11.1 Hz, 2H), 1.52 (d, J=8.7 Hz, 2H), 1.32 (t, J=7.6 Hz, 3H), 1.25 (t, J=6.9 Hz, 3H), 0.71 (s, 3H). [M+H]⁺=1025.4.

### Example 506: 3-(7-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

### Step 1: 3-(7-bromo-2-oxobenzo[d]oxazol-3(2H)-yl)piperidine-2,6-dione

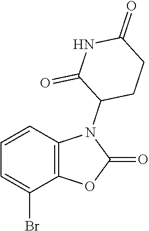 [SMILES: O=C1CCC(n2c(=O)oc3c(Br)cccc32)C(=O)N1]

A mixture of 7-bromobenzo[d]oxazol-2(3H)-one (2.13 g, 10 mmol), 3-bromopiperidine-2,6-dione (3.8 g, 20 mmol) and Cs₂CO₃ (6.5 g, 20 mmol) in DMF (50 mL) was stirred in a round bottom flask at 50° C. for 16 hours. The reaction was quenched with water (300 mL) and the mixture was extracted with DCM (100 mL×3). The combined organic layers were washed with brine (60 mL×3), dried over anhydrous Na₂SO₄, filtered and concentrated under reduced pressure. The residue was purified by re-crystallization from MeOH to afford the product (2 g, 62%), [M+H]⁺=325.1.

### Step 2: (E)-3-(7-(2-ethoxyvinyl)-2-oxobenzo[d]oxazol-3(2H)-yl)piperidine-2,6-dione

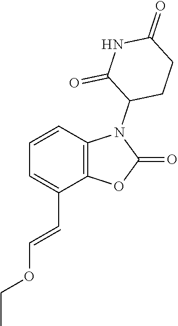 [SMILES: CCO/C=C/c1cccc2c1oc(=O)n2C1CCC(=O)NC1=O]

A mixture of 3-(7-bromo-2-oxobenzo[d]oxazol-3(2H)-yl)piperidine-2,6-dione (3.2 g, 10 mmol), (E)-2-(2-ethoxyvinyl)-4,4,5,5-tetramethyl-1,3,2-dioxaborolane (3 g, 15 mmol), Pd(Dtbpf)Cl2 (0.6 g, 1 mmol) and CsF (4.5 g, 30 mmol) in DMF (40 mL) was stirred in a round bottom flask at 100° C. overnight under nitrogen atmosphere. The reaction was quenched with water (300 mL) and the mixture was extracted with DCM (100 mL×3). The combined organic layers were washed with brine (60 mL×3), dried over anhydrous Na₂SO₄, filtered and concentrated under reduced pressure. The residue was purified by silica gel column chromatography (DCM: MeOH=100: 0˜ 10: 1 gradient elution) to afford the product (2.1 g, 68%), [M+H]⁺=317.1.

### Step 3: 2-(3-(2,6-dioxopiperidin-3-yl)-2-oxo-2,3-dihydrobenzo[d]oxazol-7-yl)acetaldehyde

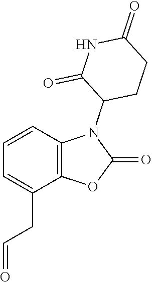 [SMILES: O=CCc1cccc2c1oc(=O)n2C1CCC(=O)NC1=O]

(E)-3-(7-(2-ethoxyvinyl)-2-oxobenzo[d]oxazol-3(2H)-yl)piperidine-2,6-dione (1.6 g, 5 mmol) in HCOOH (20 mL) was stirred in a round bottom flask at 25° C. for 2 hours. After concentration, the crude product was used directly for the next step without purification. [M+H]⁺=289.1.

### Step 4: 3-(7-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

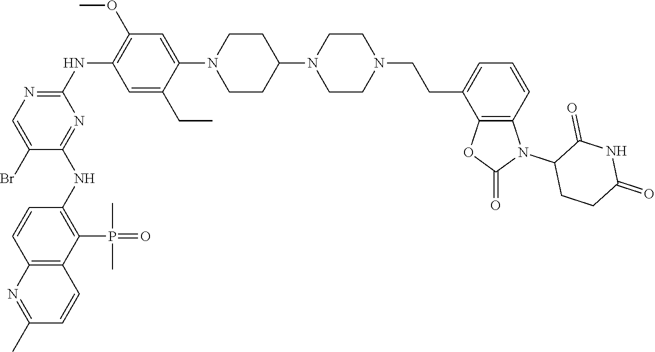 [SMILES: CCc1cc(Nc2ncc(Br)c(Nc3ccc4nc(C)ccc4c3P(C)(C)=O)n2)c(OC)cc1N1CCC(N2CCN(CCc3cccc4c3oc(=O)n4C3CCC(=O)NC3=O)CC2)CC1]

The title compound (15 mg, 30%) was prepared in a manner similar to that in Example 492 step 6 from (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide and 2-(3-(2,6-dioxopiperidin-3-yl)-2-oxo-2,3-dihydrobenzo[d]oxazol-7-yl)acetaldehyde. ¹H NMR (500 MHz, DMSO) δ 11.76 (s, 1H), 11.21 (s, 1H), 8.56 (d, J=8.7 Hz, 1H), 8.30 (s, 1H), 8.21 (s, 1H), 7.98 (s, 1H), 7.87 (d, J=9.3 Hz, 1H), 7.42 (d, J=8.8 Hz, 1H), 7.37 (s, 1H), 7.13 (dt, J=14.8, 7.5 Hz, 2H), 7.07 (d, J=7.8 Hz, 1H), 6.74 (s, 1H), 5.36 (dd, J=12.8, 5.1 Hz, 1H), 3.75 (s, 3H), 2.96-2.93 (m, 2H), 2.90-2.87 (m, 3H), 2.70-2.67 (m, 3H), 2.63-2.61 (m, 6H), 2.58-2.56 (m, 7H), 2.31-2.29 (m, 3H), 2.20-2.11 (m, 1H), 1.98 (d, J=13.3 Hz, 7H), 1.88-1.79 (m, 2H), 1.62-1.49 (m, 2H), 0.77 (s, 3H); [M+H]⁺=979.5.

### Example 507: 3-(4-(3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbon 1 azetidin-1-yl)-3,3-dimethyl-2-oxoindolin-1-yl)piperidine-2,6-dione

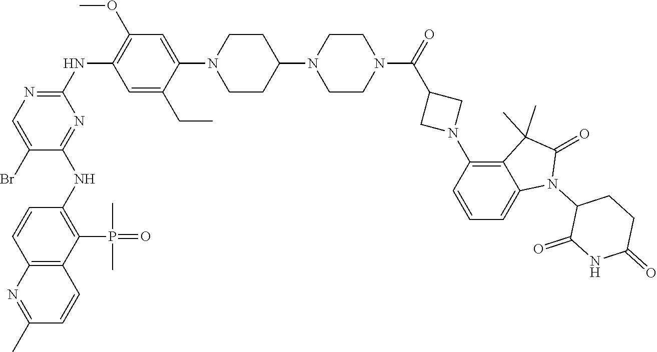 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4C(C)(C)C(=O)N5C4CCC(=O)NC4=O)C3)CC2)CC1]

The title compound (20.8 mg, 35%) was prepared in a manner similar to that in Example 447. ¹H NMR (500 MHz, DMSO) δ 11.76 (s, 1H), 11.03 (s, 1H), 8.56 (d, J=9.0 Hz, 1H), 8.30 (s, 1H), 8.21 (s, 1H), 7.98 (s, 1H), 7.87 (d, J=9.2 Hz, 1H), 7.42 (d, J=8.9 Hz, 1H), 7.38 (s, 1H), 7.10 (s, 1H), 6.74 (s, 1H), 6.47 (s, 1H), 6.28 (d, J=8.3 Hz, 1H), 5.15 (s, 1H), 4.13 (d, J=5.6 Hz, 2H), 4.08-3.95 (m, 2H), 3.77 (d, J=12.3 Hz, 4H), 3.51 (s, 2H), 3.39 (s, 2H), 2.95 (d, J=11.0 Hz, 3H), 2.72-2.62 (m, 6H), 2.56 (dd, J=13.0, 8.2 Hz, 4H), 2.43-2.34 (m, 1H), 2.30 (d, J=7.1 Hz, 2H), 2.07 (s, 1H), 1.98 (d, J=13.3 Hz, 6H), 1.93 (s, 1H), 1.83 (d, J=10.2 Hz, 2H), 1.57 (d, J=8.9 Hz, 2H), 1.35 (d, J=4.8 Hz, 6H), 0.77 (s, 3H). [M+H]⁺=1060.4.

### Example 508: 3-(4-(3-((4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)methyl)azetidin-1-yl)-2,6-difluorophenyl)piperidine-2,6-dione

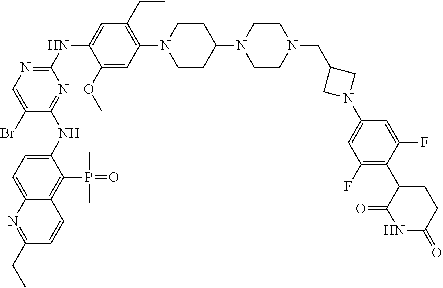 [SMILES: CCc1ccc2c(P(C)(C)=O)c(Nc3nc(Nc4cc(CC)c(N5CCC(N6CCN(CC7CN(c8cc(F)c(C9CCC(=O)NC9=O)c(F)c8)C7)CC6)CC5)cc4OC)ncc3Br)ccc2n1]

The title compound (21 mg, 31.5%) was prepared in a manner similar to that in Example 484. ¹H NMR (500 MHz, DMSO) δ 11.73 (s, 1H), 10.77 (d, J=13.3 Hz, 1H), 8.49 (d, J=8.7 Hz, 1H), 8.23 (d, J=25.9 Hz, 1H), 8.14 (s, 1H), 7.93 (s, 1H), 7.80 (d, J=9.3 Hz, 1H), 7.37 (d, J=8.8 Hz, 1H), 7.26 (s, 1H), 6.68 (s, 1H), 6.04 (d, J=11.2 Hz, 2H), 3.97-3.92 (m, 1H), 3.86 (t, J=7.7 Hz, 2H), 3.69 (s, 3H), 3.47-3.38 (m, 3H), 2.86 (dd, J=15.0, 7.6 Hz, 5H), 2.70 (dd, J=21.2, 9.1 Hz, 2H), 2.60 (t, J=11.2 Hz, 2H), 2.47 (d, J=7.4 Hz, 4H), 2.33 (s, 3H), 2.22 (d, J=6.8 Hz, 4H), 2.00 (d, J=9.0 Hz, 1H), 1.91 (d, J=13.3 Hz, 8H), 1.76 (d, J=8.9 Hz, 2H), 1.46 (d, J=8.7 Hz, 2H), 1.29-1.20 (m, 3H), 0.70 (s, 3H). [M+H]⁺=1013.4

### Example 509: 3-(7-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

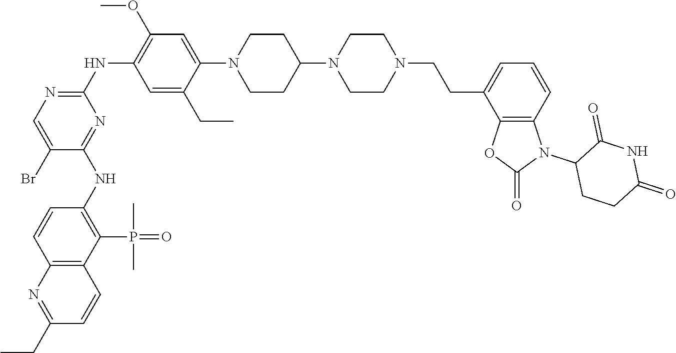 [SMILES: CCc1ccc2c(P(C)(C)=O)c(Nc3nc(Nc4cc(CC)c(N5CCC(N6CCN(CCc7cccc8c7oc(=O)n8C7CCC(=O)NC7=O)CC6)CC5)cc4OC)ncc3Br)ccc2n1]

The title compound (12 mg, 26%) was prepared in a manner similar to that in Example 492. ¹H NMR (500 MHz, DMSO) δ 11.78 (s, 1H), 11.22 (s, 1H), 8.57 (d, J=8.9 Hz, 1H), 8.27 (s, 1H), 8.21 (s, 1H), 8.00 (s, 1H), 7.88 (d, J=9.4 Hz, 1H), 7.44 (d, J=8.9 Hz, 1H), 7.35 (s, 1H), 7.19-7.02 (m, 3H), 6.75 (s, 1H), 3.76 (s, 3H), 3.01-2.82 (m, 9H), 2.79-2.53 (m, 14H), 2.29 (d, J=6.7 Hz, 2H), 2.20-2.10 (m, 1H), 1.98 (d, J=13.3 Hz, 6H), 1.90 (d, J=9.9 Hz, 2H), 1.59 (d, J=9.4 Hz, 2H), 1.32 (t, J=7.6 Hz, 3H), 0.77 (s, 3H); [M+H]⁺=993.5.

### Example 510: (R)-3-(4-(2-(4-(1-(4-((5-bromo-4-((5-(dimethylphosphoryl)-8-fluoro-2-methylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: 6-bromo-8-fluoro-2-methylquinoline

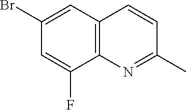

To a stirred mixture of 4-bromo-2-fluoroaniline (10 g, 52.6 mmol) in aqueous HCl (6M) (5 mL) was added (E)-but-2-enal (7.4 g, 105.3 mmol) in toluene (50 mL) dropwise at 100° C. The resulting mixture was stirred for 15 h at 100° C. under nitrogen atmosphere. The mixture was diluted with EA (50 mL) and the layers were separated. The aqueous layer was concentrated under reduced pressure.

The residue was dissolved in water (100 mL), and the pH was adjusted to 8-9 with aqueous NaOH (3M). The aqueous layer was extracted with DCM (50 mL×3). The combined organic layers were washed with brine (50 mL×3), dried over anhydrous Na₂SO₄, filtered and concentrated under reduced pressure. The crude product was purified by column chromatography (PE/EA, 0-15%) to afford product (9 g, 71.3%). [M+H]⁺=240.0.

### Step 2: N-(8-fluoro-2-methylquinolin-6-yl)-1,1-diphenylmethanimine

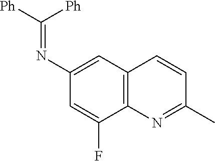 [SMILES: Cc1ccc2cc(N=C(c3ccccc3)c3ccccc3)cc(F)c2n1]

A mixture of 6-bromo-8-fluoro-2-methylquinoline (9 g, 37.5 mmol), diphenylmethanimine (7.5 g, 41.3 mmol), Pd₂(dba)₃ (1.7 g, 1.9 mmol), XantPhos (2.17 g, 3.8 mmol), and Cs₂CO₃ (30.6 g, 93.8 mmol) in dioxane (120 mL) was stirred at 100° C. under nitrogen atmosphere for 15 hrs. After cooling to r.t, the reaction mixture was filtered and concentrated in vacuo. The residue was purified by silica gel column (PE: EA=4:1) to afford product (9.5 g, 74.2%). [M+H]⁺=341.1.

### Step 3: 8-fluoro-2-methylquinolin-6-amine

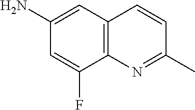

To a solution of N-(8-fluoro-2-methylquinolin-6-yl)-1,1-diphenylmethanimine (7.7 g, 22.5 mmol) in THF (50 mL) was added HCl (30 mL, 2N). The mixture was stirred at 20° C. for 30 mins and then sat. aq. Na₂CO₃ was added to PH=8-9. The resulting mixture was extracted with EtOAc (50 mL×3). The combined organic phase was washed with brine (50 mL), dried over Na₂SO₄, filtered and concentrated in vacuum. The residue was purified by silica gel column (PE: EA=1:1). 8-fluoro-2-methylquinolin-6-amine (3.2 g, 80%) was obtained. [M+H]⁺=177.1.

### Step 4: 8-fluoro-5-iodo-2-methylquinolin-6-amine

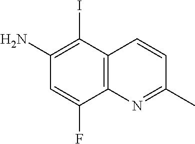

To a solution of 8-fluoro-2-methylquinolin-6-amine (1.6 g, 9.1 mmol) in HOAc (15 mL) was added ICl (1.8 g, 10.9 mmol). The mixture was stirred at 20° C. for 3 hrs and then sat. aq. Na₂CO₃ was added to PH=8-9. The resulting mixture was extracted with EtOAc (50 mL×3). The combined organic phase was washed with brine (60 mL), dried over Na₂SO₄, filtered and concentrated in vacuum. 8-fluoro-5-iodo-2-methylquinolin-6-amine (2.1 g, 76.3%) was obtained. [M+H]⁺=303.0.

### Step 5: (6-amino-8fluoro-2-methylquinolin-5-yl)dimethylphosphine oxide

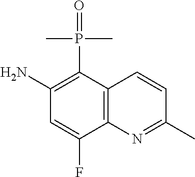 [SMILES: Cc1ccc2c(P(C)(C)=O)c(N)cc(F)c2n1]

A mixture of 8-fluoro-5-iodo-2-methylquinolin-6-amine (2.1 g, 7 mmol), dimethylphosphineoxide (819 mg, 10.5 mmol), K₃PO₄ (3.7 g, 17.5 mmol), Pd(OAc)₂ (160 mg, 0.7 mmol) and XantPhos (420 mg, 0.7 mmol) in dioxane (30 mL) was stirred for 3 h at 100° C. under nitrogen atmosphere. After cooling to room temperature, the mixture was concentrated in vacuum, the residue was purified by column chromatography (0-10% MeOH in DCM) to afford the product (1.2 g, 68.2%). [M+H]⁺=253.1.

### Step 6: (6-((5-bromo-2-chloropyrimidin-4-yl)amino)-8-fluoro-2-methylquinolin-5-yl)dimethylphosphine oxide

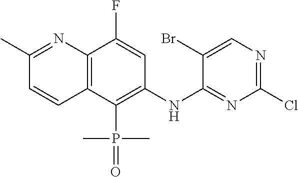 [SMILES: Cc1ccc2c(P(C)(C)=O)c(Nc3nc(Cl)ncc3Br)cc(F)c2n1]

To a solution of (6-amino-8-fluoro-2-methylquinolin-5-yl)dimethylphosphine oxide (1.2 g, 4.8 mmol) in THF (30 mL) was added 5-bromo-2,4-dichloropyrimidine (2.8 g, 11.9 mmol). LiHMDS (1 M in THF, 11.9 mL, 11.9 mmol) was added to the reaction mixture at 0° C. The mixture was stirred at 20° C. for 3 hrs. The mixture was diluted with water (20 mL) and the layers were separated. The aqueous layer was extracted with DCM (20 mL×3). The combined organic layers were washed with brine (20 mL×3), dried over Na₂SO₄, filtered and concentrated under reduced pressure. The residue was purified by column chromatography (DCM/MeOH=20/1 to 10/1) to afford product (820 mg, 39.1%). [M+H]⁺=443.0.

### Step 7: (6-((5-bromo-2-((5-ethyl-2-methoxy-4-(4-(piperazin-1-yl)piperidin-1-yl)phenyl)amino)pyrimidin-4-yl)amino)-8-fluoro-2-methylquinolin-5-yl)dimethylphosphine oxide

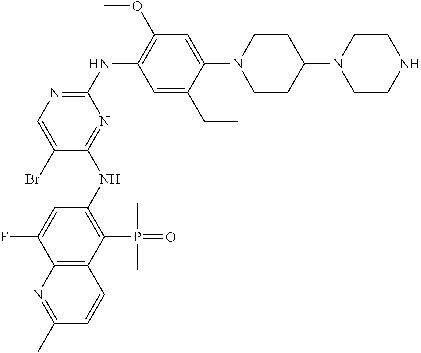 [SMILES: CCc1cc(Nc2ncc(Br)c(Nc3cc(F)c4nc(C)ccc4c3P(C)(C)=O)n2)c(OC)cc1N1CCC(N2CCNCC2)CC1]

To a stirred solution of (6-((5-bromo-2-chloropyrimidin-4-yl)amino)-8-fluoro-2-methylquinolin-5-yl)dimethylphosphine oxide (820 mg, 1.85 mmol) and tert-butyl 4-(1-(4-amino-2-ethyl-5-methoxyphenyl)piperidin-4-yl)piperazine-1-carboxylate (775 mg, 1.85 mmol) in n-BuOH (40 mL) was added TsOH (890 mg, 5.6 mmol). The resulting mixture was stirred at 95° C. for 16 hours. The reaction mixture was concentrated under reduced pressure and the residue was dissolved in aqueous NaOH (1M, 10 mL). Then the aqueous layer was extracted with DCM (2×20 mL). The combined organic layer was washed with brine (2×20 mL), dried over Na₂SO₄, filtered and concentrated under vacuum to afford the crude residue, which was purified with silica gel column chromatography (DCM: MeOH=6:1) to give the title product (710 mg, 58.5%). [M+H]⁺=725.2.

### Step 8: (R)-3-(4-(2-(4-(1-(4-((5-bromo-4-((5-(dimethylphosphoryl)-8-fluoro-2-methylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2, 6-dione

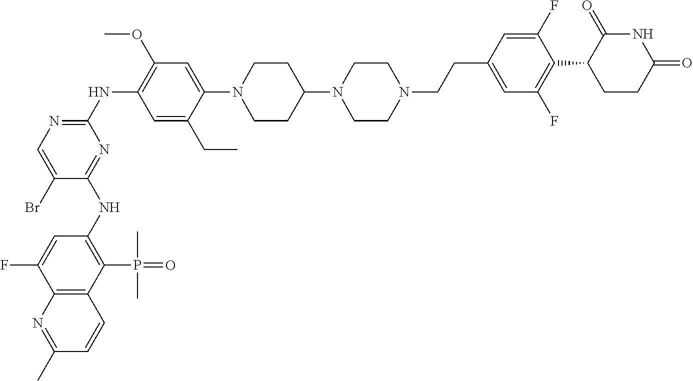 [SMILES: CCc1cc(Nc2ncc(Br)c(Nc3cc(F)c4nc(C)ccc4c3P(C)(C)=O)n2)c(OC)cc1N1CCC(N2CCN(CCc3cc(F)c([C@H]4CCC(=O)NC4=O)c(F)c3)CC2)CC1]

The title compound (15 mg, 24%) was prepared in a manner similar to that in Example 484 step 15 from (6-((5-bromo-2-((5-ethyl-2-methoxy-4-(4-(piperazin-1-yl)piperidin-1-yl)phenyl)amino)pyrimidin-4-yl)amino)-8-fluoro-2-methylquinolin-5-yl)dimethylphosphine oxide and (R)-2-(4-(2,6-dioxopiperidin-3-yl)-3,5-difluorophenyl)acetaldehyde. ¹H NMR (500 MHz, DMSO) δ 12.16 (s, 1H), 10.95 (s, 1H), 8.53 (d, J=8.5 Hz, 1H), 8.38 (s, 1H), 8.23 (d, J=2.7 Hz, 1H), 8.15 (s, 1H), 7.51 (d, J=9.0 Hz, 1H), 7.34 (s, 1H), 7.03 (d, J=12.1 Hz, 2H), 6.74 (s, 1H), 4.20 (d, J=13.3 Hz, 1H), 3.77 (d, J=2.8 Hz, 3H), 2.97-2.95 (m, 2H), 2.81-2.76 (m, 3H), 2.70-2.53 (m, 13H), 2.50-2.35 (m, 6H), 2.17-2.09 (m, 1H), 2.05-1.94 (m, 7H), 1.86-1.84 (m, 2H), 1.55 (d, J=11.7 Hz, 2H), 0.82 (s, 3H). [M+H]⁺=976.4

### Example 511: (R)-3-(4-((R)-3-(4-(1-(4-((5-bromo-4-((5-(dimethylphosphoryl)-8-fluoro-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

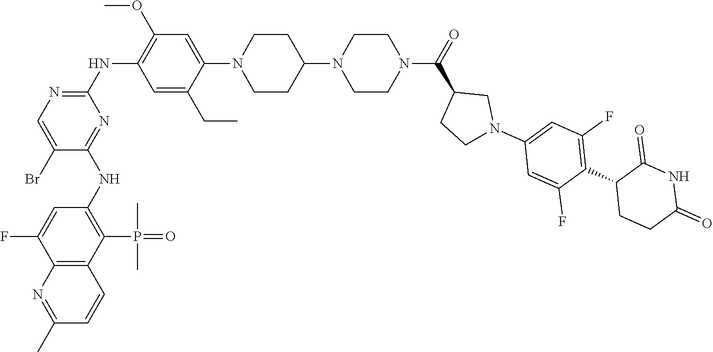 [SMILES: CCc1cc(Nc2ncc(Br)c(Nc3cc(F)c4nc(C)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound was prepared in a manner similar to that in Example 488. ¹H NMR (500 MHz, DMSO) δ 12.16 (s, 1H), 10.84 (s, 1H), 8.52 (d, J=8.8 Hz, 1H), 8.40 (d, J=11.3 Hz, 1H), 8.23 (s, 1H), 8.15 (d, J=8.2 Hz, 1H), 7.51 (d, J=9.0 Hz, 1H), 7.35 (s, 1H), 6.75 (s, 1H), 6.23 (d, J=12.1 Hz, 2H), 4.05-3.96 (m, 1H), 3.77 (s, 3H), 3.62-3.40 (m, 7H), 3.35-3.33 (m, 2H), 3.30-3.17 (m, 2H), 2.99-2.97 (m, 2H), 2.84-2.72 (m, 1H), 2.71-2.54 (m, 7H), 2.50-2.35 (m, 4H), 2.23-1.90 (m, 10H), 1.85-1.83 (m, 2H), 1.66-1.49 (m, 2H), 0.82 (s, 3H). [M+H]⁺=1045.6.

### Example 512: 3-(4-(4-(4-(1-(4-((5-bromo-4-((5-(dimethylphosphoryl)-8-fluoro-2-methylquinolin-6-yl)amino)pyrimidin-2-yl)amino)-2-ethyl-5-methoxyphenyl)piperidin-4-yl)piperazine-1-carbonyl)piperidin-1-yl)-2,6-difluorophenyl)piperidine-2,6-dione

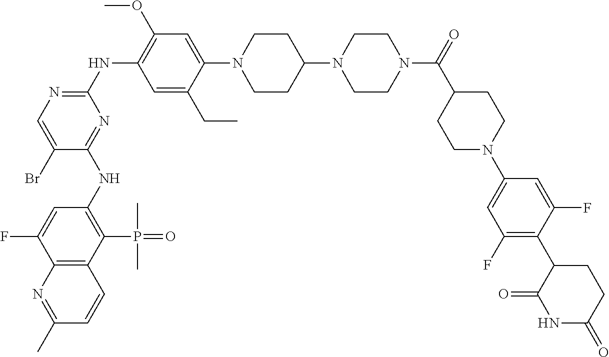 [SMILES: CCc1cc(Nc2ncc(Br)c(Nc3cc(F)c4nc(C)ccc4c3P(C)(C)=O)n2)c(OC)cc1N1CCC(N2CCN(C(=O)C3CCN(c4cc(F)c(C5CCC(=O)NC5=O)c(F)c4)CC3)CC2)CC1]

The title compound was prepared in a manner similar to that in Example 488.

¹H NMR (500 MHz, DMSO) δ 12.16 (s, 1H), 10.87 (s, 1H), 8.52 (d, J=8.8 Hz, 1H), 8.39 (d, J=10.6 Hz, 1H), 8.25-8.11 (m, 2H), 7.51 (d, J=8.9 Hz, 1H), 7.34 (s, 1H), 6.74 (s, 1H), 6.63 (d, J=12.8 Hz, 2H), 4.05 (dd, J=12.6, 4.9 Hz, 1H), 3.80-3.76 (m, 5H), 3.54-3.46 (m, 5H), 2.98-2.96 (m, 2H), 2.91-2.72 (m, 4H), 2.72-2.51 (m, 8H), 2.50-2.29 (m, 4H), 2.16-1.91 (m, 8H), 1.84-1.82 (m, 2H), 1.71-1.49 (m, 6H), 0.81 (s, 3H). [M+H]⁺=1059.6.

### Example 513: 3-(4-((S)-3-(4-(1-(4-((5-bromo-4-((5-(dimethylphosphoryl)-2-methyl-1-oxo-1,2-dihydrophthalazin-6-yl)amino)pyrimidin-2-yl)amino)-5-methoxy-2-methylphenyl)piperidin-4-yl)piperazine-1-carbonyl)pyrrolidin-1-yl)-2,6-difluorophenyl)piperidine-2,6-dione

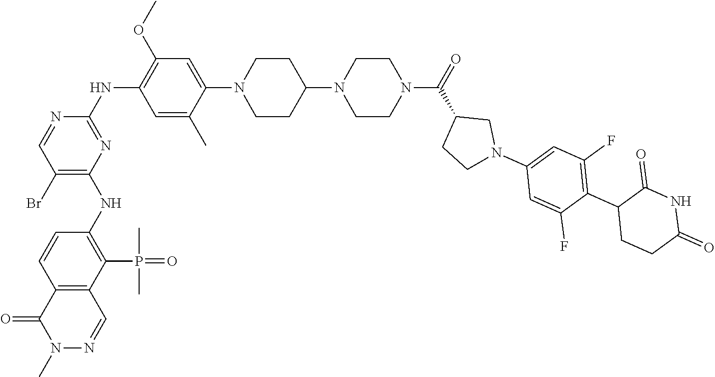 [SMILES: COc1cc(N2CCC(N3CCN(C(=O)[C@H]4CCN(c5cc(F)c(C6CCC(=O)NC6=O)c(F)c5)C4)CC3)CC2)c(C)cc1Nc1ncc(Br)c(Nc2ccc3c(=O)n(C)ncc3c2P(C)(C)=O)n1]

The title compound (16 mg, 35%) was prepared in a manner similar to that in Example 488. ¹H NMR (500 MHz, DMSO) δ 12.34 (s, 1H), 10.84 (s, 1H), 8.62 (s, 1H), 8.54 (s, 1H), 8.24 (s, 1H), 8.20-8.15 (m, 2H), 7.31 (s, 1H), 6.71 (s, 1H), 6.23 (d, J=12.1 Hz, 2H), 4.02 (dd, J=12.5, 5.0 Hz, 1H), 3.75 (s, 3H), 3.72 (s, 3H), 3.53-3.43 (m, 9H), 3.28-3.18 (m, 4H), 3.09 (d, J=10.8 Hz, 2H), 2.82 -2.73 (m, 1H), 2.65 (t, J=11.1 Hz, 2H), 2.59 (s, 2H), 2.41 (t, J=11.1 Hz, 1H), 2.21-2.02 (m, 11H), 1.98-1.92 (m, 1H), 1.86 (d, J=10.5 Hz, 2H), 1.65-1.58 (m, 2H). [M+H]⁺=1030.4.

### Example 514: 3-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-3,3-dimethyl-2-oxoindolin-1-yl)piperidine-2,6-dione

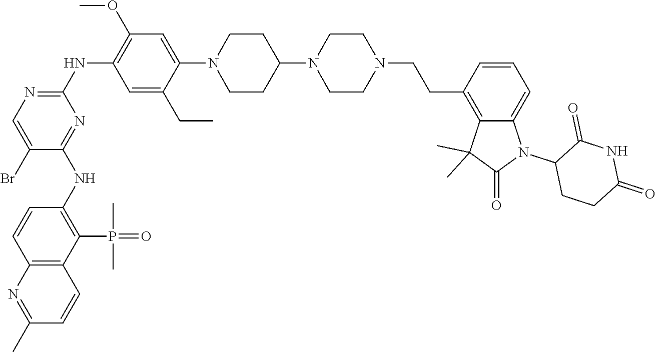 [SMILES: CCc1cc(Nc2ncc(Br)c(Nc3ccc4nc(C)ccc4c3P(C)(C)=O)n2)c(OC)cc1N1CCC(N2CCN(CCc3cccc4c3C(C)(C)C(=O)N4C3CCC(=O)NC3=O)CC2)CC1]

The title compound (20 mg, 29%) was prepared in a manner similar to that in Example 413. ¹H NMR (500 MHz, DMSO) δ 11.70 (s, 1H), 11.00 (s, 1H), 8.49 (d, J=8.9 Hz, 1H), 8.24 (s, 1H), 8.11 (s, 1H), 7.91 (s, 1H), 7.80 (d, J=9.3 Hz, 1H), 7.42-7.25 (m, 2H), 7.11 (t, J=7.8 Hz, 1H), 6.85 (d, J=7.7 Hz, 1H), 6.76 (s, 1H), 6.68 (s, 1H), 5.14 (s, 1H), 3.70 (d, J=11.9 Hz, 4H), 2.87 (d, J=10.4 Hz, 2H), 2.77 (dd, J=18.7, 11.2 Hz, 4H), 2.60 (d, J=16.3 Hz, 6H), 2.55 (s, 1H), 2.53-2.45 (m, 7H), 2.23 (d, J=8.3 Hz, 3H), 1.92 (d, J=13.3 Hz, 7H), 1.78 (d, J=10.9 Hz, 2H), 1.49 (dd, J=20.3, 11.0 Hz, 2H), 1.33 (t, J=11.0 Hz, 7H), 0.71 (s, 3H). [M+H]⁺=1005.4

### Example 515: 3-(4-((R)-3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

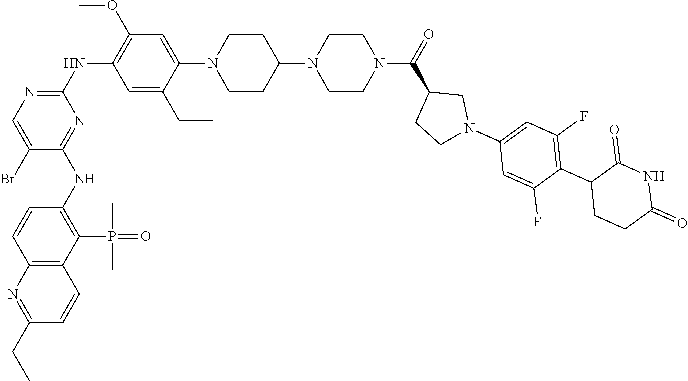 [SMILES: CCc1ccc2c(P(C)(C)=O)c(Nc3nc(Nc4cc(CC)c(N5CCC(N6CCN(C(=O)[C@@H]7CCN(c8cc(F)c(C9CCC(=O)NC9=O)c(F)c8)C7)CC6)CC5)cc4OC)ncc3Br)ccc2n1]

The title compound (42.5 mg, 55%) was prepared in a manner similar to that in Example 488. ¹H NMR (500 MHz, DMSO) δ 11.73 (s, 1H), 10.78 (s, 1H), 8.49 (d, J=8.9 Hz, 1H), 8.19-8.16 (m, 2H), 7.94 (s, 1H), 7.81 (d, J=9.1 Hz, 1H), 7.37 (d, J=8.8 Hz, 1H), 7.27 (s, 1H), 6.69 (s, 1H), 6.16 (d, J=12.3 Hz, 2H), 3.95 (dd, J=12.4, 4.5 Hz, 1H), 3.69 (s, 3H), 3.55-3.33 (m, 7H), 3.18 (d, J=7.8 Hz, 4H), 2.94-2.82 (m, 4H), 2.75-2.67 (m, 1H), 2.61 (t, J=11.0 Hz, 2H), 2.49 (d, J=12.2 Hz, 4H), 2.36-2.18 (m, 2H), 2.15-1.98 (m, 3H), 1.92 (d, J=13.3 Hz, 7H), 1.77 (d, J=10.7 Hz, 2H), 1.51 (d, J=9.9 Hz, 2H), 1.25 (t, J=7.5 Hz, 3H), 0.71 (s, 3H). [M+H]⁺=1041.4.

### Example 516: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-3-methyl-2-oxo-2,3-dihydro-1H-benzo[d]imidazol-1-yl)piperidine-2,6-dione

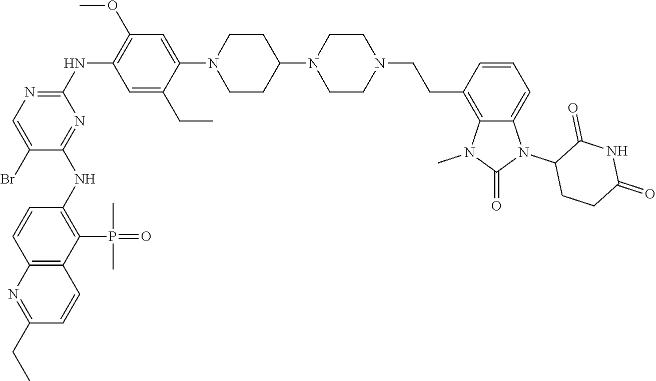 [SMILES: CCc1ccc2c(P(C)(C)=O)c(Nc3nc(Nc4cc(CC)c(N5CCC(N6CCN(CCc7cccc8c7n(C)c(=O)n8C7CCC(=O)NC7=O)CC6)CC5)cc4OC)ncc3Br)ccc2n1]

The title compound (50 mg, 65%) was prepared in a manner similar to that in Example 318. ¹H NMR (500 MHz, DMSO) δ 11.80 (s, 1H), 11.09 (s, 1H), 8.56 (d, J=8.8 Hz, 1H), 8.30 (s, 1H), 8.21 (s, 1H), 8.01 (s, 1H), 7.87 (d, J=9.3 Hz, 1H), 7.44 (d, J=8.9 Hz, 1H), 7.33 (s, 1H), 7.02-6.94 (m, 2H), 6.91 (dd, J=6.2, 2.6 Hz, 1H), 6.76 (s, 1H), 5.37 (dd, J=12.6, 5.3 Hz, 1H), 3.76 (s, 3H), 3.59 (s, 3H), 3.11-3.04 (m, 2H), 2.96-2.91 (m, 6H), 2.77-2.63 (m, 5H), 2.57 (dd, J=20.5, 12.4 Hz, 8H), 2.29 (d, J=4.6 Hz, 3H), 1.98 (d, J=13.3 Hz, 7H), 1.85 (d, J=10.9 Hz, 2H), 1.55 (d, J=9.0 Hz, 2H), 1.32 (t, J=7.6 Hz, 3H), 0.78 (s, 3H). [M+H]⁺=1006.4

### Example 517: 3-(4-(4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)piperidin-1-yl)-2,6-difluorophenyl)piperidine-2,6-dione

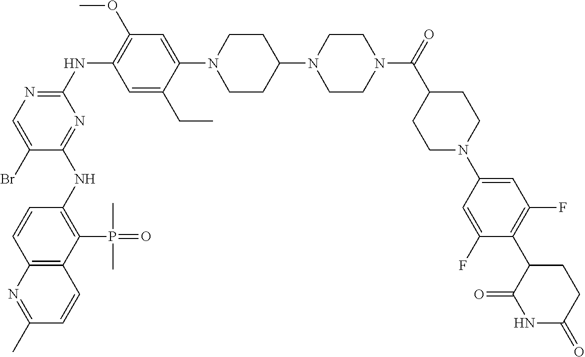 [SMILES: CCc1cc(Nc2ncc(Br)c(Nc3ccc4nc(C)ccc4c3P(C)(C)=O)n2)c(OC)cc1N1CCC(N2CCN(C(=O)C3CCN(c4cc(F)c(C5CCC(=O)NC5=O)c(F)c4)CC3)CC2)CC1]

The title compound (14.71 mg, 14%) was prepared in a manner similar to that in Example 488. ¹H NMR (500 MHz, DMSO) δ 11.76 (s, 1H), 10.87 (s, 1H), 8.56 (d, J=9.0 Hz, 1H), 8.30 (d, J=9.7 Hz, 1H), 8.21 (s, 1H), 7.97 (s, 1H), 7.87 (d, J=9.3 Hz, 1H), 7.42 (d, J=8.9 Hz, 1H), 7.38 (s, 1H), 6.74 (s, 1H), 6.63 (d, J=12.8 Hz, 2H), 4.05 (dd, J=12.5, 5.0 Hz, 1H), 3.84-3.70 (m, 5H), 3.54 (s, 2H), 3.46 (s, 2H), 2.95 (d, J=10.9 Hz, 2H), 2.90-2.73 (m, 4H), 2.71-2.61 (m, 5H), 2.55 (d, J=4.7 Hz, 3H), 2.45 (s, 2H), 2.41-2.33 (m, 1H), 2.30 (dd, J=13.3, 6.0 Hz, 2H), 2.15-2.03 (m, 1H), 2.03-1.91 (m, 7H), 1.83 (d, J=10.2 Hz, 2H), 1.72-1.49 (m, 6H), 0.93-0.60 (m, 3H). [M+H]⁺=1041.5.

### Example 518: 3-(4-(4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)piperidin-1-yl)-2,6-difluorophenyl)piperidine-2,6-dione

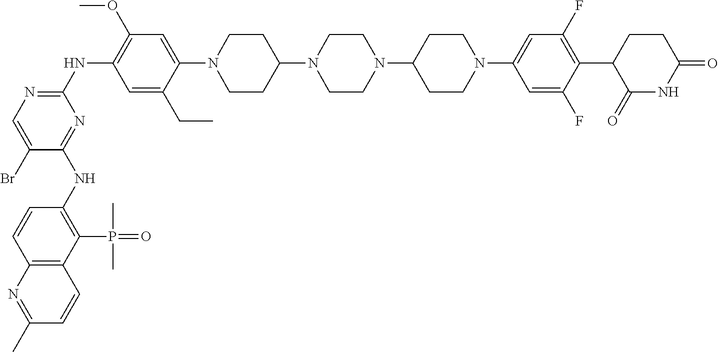 [SMILES: CCc1cc(Nc2ncc(Br)c(Nc3ccc4nc(C)ccc4c3P(C)(C)=O)n2)c(OC)cc1N1CCC(N2CCN(C3CCN(c4cc(F)c(C5CCC(=O)NC5=O)c(F)c4)CC3)CC2)CC1]

The title compound (7.36 mg, 7%) was prepared in a manner similar to that in Example 484. ¹H NMR (500 MHz, DMSO) δ 11.76 (s, 1H), 10.87 (s, 1H), 8.56 (d, J=9.0 Hz, 1H), 8.30 (d, J=5.0 Hz, 1H), 8.21 (s, 1H), 7.97 (s, 1H), 7.87 (d, J=9.1 Hz, 1H), 7.42 (d, J=8.9 Hz, 1H), 7.37 (s, 1H), 6.74 (s, 1H), 6.62 (d, J=12.8 Hz, 2H), 4.04 (dd, J=12.4, 5.0 Hz, 1H), 3.82-3.70 (m, 5H), 3.28-3.23 (m, 2H), 2.93 (d, J=10.6 Hz, 2H), 2.83-2.70 (m, 3H), 2.69-2.59 (m, 6H), 2.59-2.52 (m, 6H), 2.35-2.21 (m, 4H), 2.14-2.03 (m, 1H), 2.02-1.91 (m, 7H), 1.88-1.75 (m, 4H), 1.53 (dd, J=20.6, 10.4 Hz, 2H), 1.43 (dd, J=21.0, 10.8 Hz, 2H), 0.88-0.65 (m, 3H). [M+H]⁺=1013.7.

### Example 519: 3-(7-(3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)azetidin-1-yl)-2-oxobenzo[d]oxazol-3(2H)-yl)piperidine-2,6-dione

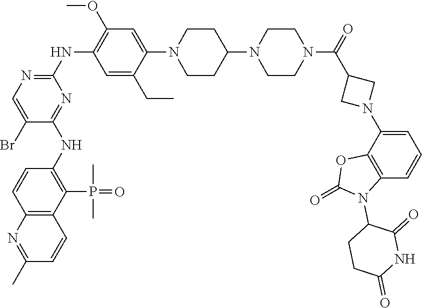 [SMILES: CCc1cc(Nc2ncc(Br)c(Nc3ccc4nc(C)ccc4c3P(C)(C)=O)n2)c(OC)cc1N1CCC(N2CCN(C(=O)C3CN(c4cccc5c4oc(=O)n5C4CCC(=O)NC4=O)C3)CC2)CC1]

The title compound (13 mg, 26%) was prepared in a manner similar to that in Example 447. ¹H NMR (500 MHz, DMSO) δ 11.76 (s, 1H), 11.19 (s, 1H), 8.56 (d, J=8.9 Hz, 1H), 8.30 (s, 1H), 8.22 (d, J=6.6 Hz, 1H), 7.98 (s, 1H), 7.87 (d, J=9.3 Hz, 1H), 7.44-7.33 (m, 2H), 7.03 (t, J=8.1 Hz, 1H), 6.74 (s, 1H), 6.61 (d, J=7.9 Hz, 1H), 6.31 (d, J=8.2 Hz, 1H), 5.30 (dd, J=13.0, 5.5 Hz, 1H), 4.21 (t, J=7.8 Hz, 2H), 4.08 (t, J=6.8 Hz, 2H), 3.88 (dd, J=14.9, 7.6 Hz, 1H), 3.74 (d, J=15.2 Hz, 3H), 3.51 (d, J=13.9 Hz, 2H), 3.01-2.79 (m, 3H), 2.74-2.65 (m, 4H), 2.58-2.51 (m, 5H), 2.48-2.43 (m, 2H), 2.42-2.23 (m, 4H), 2.19-2.09 (m, 1H), 1.97 (t, J=14.3 Hz, 7H), 1.83 (d, J=10.3 Hz, 2H), 1.57 (d, J=8.7 Hz, 2H), 0.77 (s, 3H); [M+H]⁺=1034.5.

### Example 520: 3-(4-(3-(4-(1-(4-((5-bromo-4-((5-(dimethylphosphoryl)-2-methyl-1-oxo-1,2-dihydrophthalazin-6-yl)amino)pyrimidin-2-yl)amino)-2-ethyl-5-methoxyphenyl)piperidin-4-yl)piperazine-1-carbonyl)azetidin-1-yl)-2,6-difluorophenyl)piperidine-2,6-dione

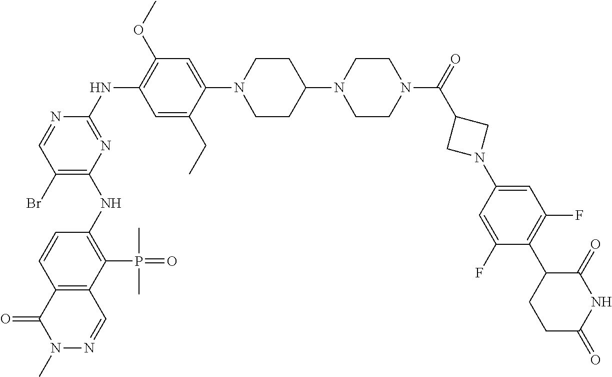 [SMILES: CCc1cc(Nc2ncc(Br)c(Nc3ccc4c(=O)n(C)ncc4c3P(C)(C)=O)n2)c(OC)cc1N1CCC(N2CCN(C(=O)C3CN(c4cc(F)c(C5CCC(=O)NC5=O)c(F)c4)C3)CC2)CC1]

The title compound (8 mg, 20%) was prepared in a manner similar to that in Example 488. ¹H NMR (500 MHz, DMSO) δ 12.20 (s, 1H), 10.86 (s, 1H), 8.57 (s, 2H), 8.27-8.09 (m, 3H), 7.34 (s, 1H), 6.76 (s, 1H), 6.17 (d, J=11.1 Hz, 2H), 4.04 (s, 2H), 3.94-3.90 (m, 2H), 3.86-3.81 (m, 2H), 3.75 (s, 3H), 3.72 (s, 3H), 3.49 (s, 4H), 2.99 (d, J=9.3 Hz, 2H), 2.83-2.64 (m, 6H), 2.42 (s, 3H), 2.12-1.91 (m, 10H), 1.84 (d, J=10.4 Hz, 2H), 1.65-1.54 (m, 2H), 0.92 (s, 3H). [M+H]⁺=1030.4.

### Example 521: 3-(4-(2-(4-(1-(4-((5-bromo-4-((2-cyclopropyl-5-(dimethylphosphoryl)-1-oxo-1,2-dihydrophthalazin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

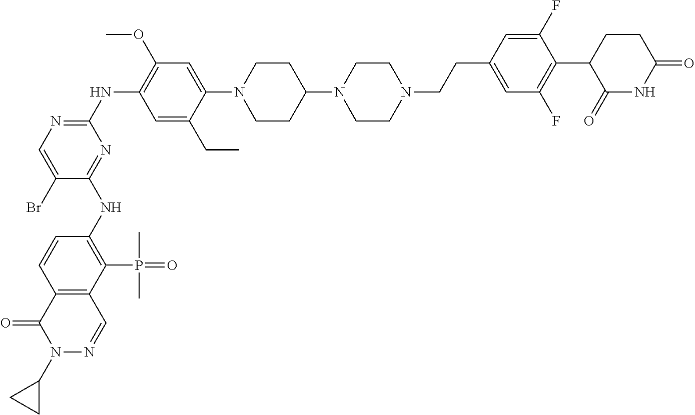 [SMILES: CCc1cc(Nc2ncc(Br)c(Nc3ccc4c(=O)n(C5CC5)ncc4c3P(C)(C)=O)n2)c(OC)cc1N1CCC(N2CCN(CCc3cc(F)c(C4CCC(=O)NC4=O)c(F)c3)CC2)CC1]

The title compound (12 mg, 20%) was prepared in a manner similar to that in Example 484. ¹H NMR (500 MHz, DMSO) δ 12.22 (s, 1H), 10.95 (s, 1H), 8.56 (s, 2H), 8.20-8.17 (m, 3H), 7.33 (s, 1H), 7.03 (d, J=10.1 Hz, 2H), 6.76 (s, 1H), 4.20 (dd, J=12.6, 4.9 Hz, 1H), 4.05-4.03 (m, 1H), 3.74-3.72 (m, 3H), 2.99 (d, J=10.0 Hz, 2H), 2.85-2.72 (m, 3H), 2.68 (t, J=11.0 Hz, 2H), 2.58-2.53 (m, 7H), 2.49-2.37 (m, 6H), 2.36-2.26 (m, 1H), 2.19-2.07 (m, 1H), 2.01 (d, J=13.2 Hz, 7H), 1.85 (d, J=11.4 Hz, 2H), 1.57 (dd, J=20.1, 11.2 Hz, 2H), 1.03 (d, J=12.0 Hz, 2H), 1.00-0.95 (m, 2H), 0.92 (s, 3H); [M+H]⁺=1001.5.

### Example 522: 3-(6-(2-(4-(1-(4-((5-bromo-4-((2-cyclopropyl-5-(dimethylphosphoryl)-1-oxo-1,2-dihydrophthalazin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

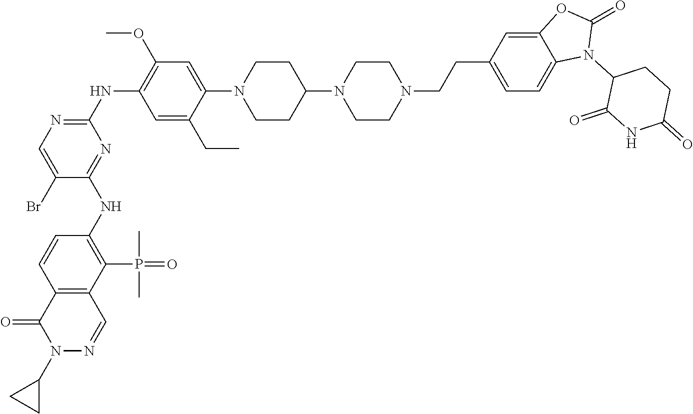 [SMILES: CCc1cc(Nc2ncc(Br)c(Nc3ccc4c(=O)n(C5CC5)ncc4c3P(C)(C)=O)n2)c(OC)cc1N1CCC(N2CCN(CCc3ccc4c(c3)oc(=O)n4C3CCC(=O)NC3=O)CC2)CC1]

The title compound (16 mg, 22%) was prepared in a manner similar to that in Example 492. ¹H NMR (500 MHz, DMSO) δ 12.23 (s, 1H), 11.20 (s, 1H), 8.55 (s, 2H), 8.25 (s, 1H), 8.17 (d, J=8.6 Hz, 2H), 7.30 (s, 2H), 7.16 (d, J=8.1 Hz, 1H), 7.09-7.02 (m, 1H), 6.76 (s, 1H), 5.35 (dd, J=12.9, 5.2 Hz, 1H), 4.06-4.03 (m, 1H), 3.78-3.75 (m, 3H), 2.99-2.97 (m, 2H), 2.91-2.87 (m, 1H), 2.78-2.76 (m, 2H), 2.73-2.61 (m, 5H), 2.55-2.53 (m, 5H), 2.48-2.38 (m, 6H), 2.32-2.28 (m, 1H), 2.16-2.14 (m, 1H), 2.06-2.03 (m, 6H), 1.88-1.85 (m, 2H), 1.58-1.55 (m, 2H), 1.06-0.82 (m, 7H); [M+H]⁺=1022.5.

### Example 523: 3-(6-(2-(4-(1-(4-((5-bromo-4-((5-(dimethylphosphoryl)-2-methyl-1-oxo-1,2-dihydrophthalazin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

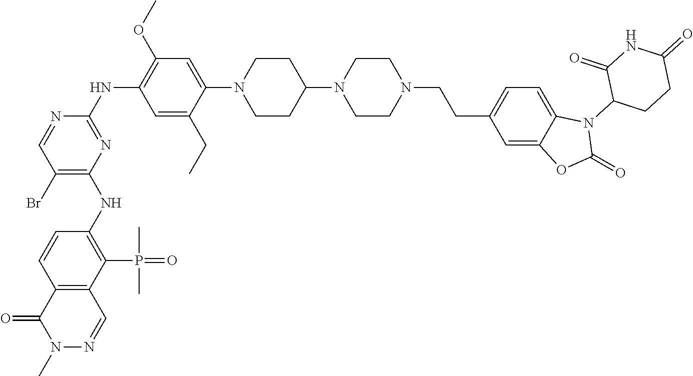 [SMILES: CCc1cc(Nc2ncc(Br)c(Nc3ccc4c(=O)n(C)ncc4c3P(C)(C)=O)n2)c(OC)cc1N1CCC(N2CCN(CCc3ccc4c(c3)oc(=O)n4C3CCC(=O)NC3=O)CC2)CC1]

The title compound (10 mg, 30%) was prepared in a manner similar to that in Example 492. ¹H NMR (500 MHz, DMSO) δ 12.14 (s, 1H), 11.13 (s, 1H), 8.50 (s, 2H), 8.22-8.06 (m, 3H), 7.23 (s, 2H), 7.09 (d, J=8.1 Hz, 1H), 7.01 (d, J=8.1 Hz, 1H), 6.69 (s, 1H), 5.29 (s, 1H), 3.68 (s, 3H), 3.65 (s, 3H), 2.92 (d, J=9.4 Hz, 2H), 2.86-2.79 (m, 1H), 2.71 (d, J=7.2 Hz, 2H), 2.65-2.55 (m, 5H), 2.46 (d, J=8.6 Hz, 5H), 2.36 (s, 7H), 2.09 (d, J=10.6, 1H), 1.96 (d, J=13.2 Hz, 6H), 1.78 (d, J=10.6 Hz, 2H), 1.50 (d, J=8.8 Hz, 2H), 0.85 (s, 3H). [M+H]⁺=996.4.

### Example 525: 3-(6-((R)-3-((4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)methyl)pyrrolidin-1-yl)-2-oxobenzo[d]oxazol-3(2H)-yl)piperidine-2,6-dione

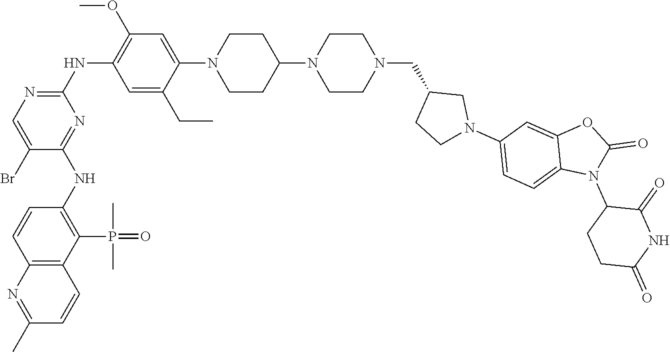 [SMILES: CCc1cc(Nc2ncc(Br)c(Nc3ccc4nc(C)ccc4c3P(C)(C)=O)n2)c(OC)cc1N1CCC(N2CCN(C[C@H]3CCN(c4ccc5c(c4)oc(=O)n5C4CCC(=O)NC4=O)C3)CC2)CC1]

The title compound (9.05 mg, 12.7%) was prepared in a manner similar to that in Example 492. ¹H NMR (500 MHz, DMSO) δ 11.86-11.58 (m, 1H), 11.16 (s, 1H), 8.56 (d, J=8.8 Hz, 1H), 8.29 (s, 1H), 8.22 (d, J=11.8 Hz, 1H), 7.97 (s, 1H), 7.87 (d, J=9.2 Hz, 1H), 7.48-7.27 (m, 2H), 7.03 (d, J=8.6 Hz, 1H), 6.74 (s, 1H), 6.60-6.58 (m, 1H), 6.33 (dd, J=8.7, 2.1 Hz, 1H), 5.26 (dd, J=13.1, 5.3 Hz, 1H), 4.34 (d, J=4.6 Hz, 2H), 3.76 (s, 3H), 3.52-3.40 (m, 4H), 3.03-2.76 (m, 5H), 2.69-2.63 (m, 7H), 2.57-2.54 (m, 3H), 2.40-2.21 (m, 7H), 2.18-2.03 (m, 2H), 2.00 (s, 3H), 1.98 (s, 3H), 1.86-1.82 (m, 2H), 1.75-1.66 (m, 1H), 1.57-1.53 (m, 2H), 0.77 (s, 3H). [M+H]⁺=1034.5.

### Example 526: (R)-3-(4-((R)-3-(4-(1-(4-((5-bromo-4-((2-cyclopropyl-5-(dimethylphosphoryl)-1-oxo-1,2-dihydrophthalazin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

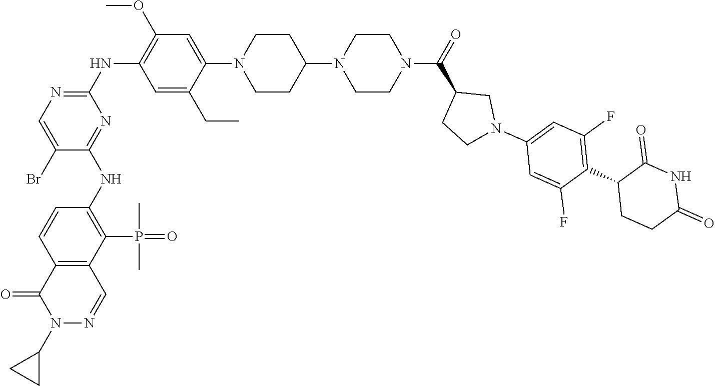 [SMILES: CCc1cc(Nc2ncc(Br)c(Nc3ccc4c(=O)n(C5CC5)ncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound (20 mg, 26%) was prepared in a manner similar to that in Example 488. ¹H NMR (500 MHz, DMSO) δ 12.23 (s, 1H), 10.84 (s, 1H), 8.55 (s, 2H), 8.25 (s, 1H), 8.19 (d, J=16.1 Hz, 2H), 7.30 (s, 1H), 6.77 (s, 1H), 6.23 (d, J=12.1 Hz, 2H), 4.07-3.97 (m, 2H), 3.75 (s, 3H), 3.60-3.40 (m, 7H), 3.34 (d, J=9.8 Hz, 1H), 3.30-3.21 (m, 4H), 3.06-2.93 (m, 2H), 2.77 (t, J=7.1 Hz, 1H), 2.69-2.67 (m, 2H), 2.59-2.58 (m, 2H), 2.42-2.39 (m, 4H), 2.15-2.10 (m, 1H), 2.09-2.07 (m, 2H), 2.04-2.02 (m, 5H), 1.96-1.91 (m, 1H), 1.89-1.81 (m, 2H), 1.62-1.60 (m, 2H), 1.02 (s, 2H), 1.00-0.85 (m, 5H); [M+H]⁺=1070.5.

### Example 527: (R)-3-(4-((R)-3-(4-(1-(4-((5-bromo-4-((5-(dimethylphosphoryl)-2-methyl-1-oxo-1,2-dihydrophthalazin-6-yl)amino)pyrimidin-2-yl)amino)-5-methoxy-2-methylphenyl)piperidin-4-yl)piperazine-1-carbonyl)pyrrolidin-1-yl)-2,6-difluorophenyl)piperidine-2,6-dione

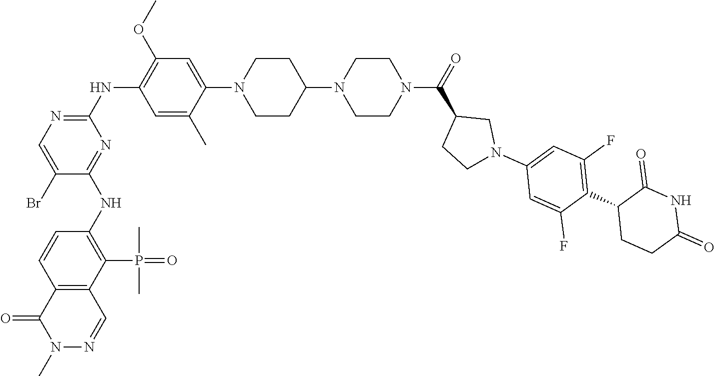 [SMILES: COc1cc(N2CCC(N3CCN(C(=O)[C@@H]4CCN(c5cc(F)c([C@H]6CCC(=O)NC6=O)c(F)c5)C4)CC3)CC2)c(C)cc1Nc1ncc(Br)c(Nc2ccc3c(=O)n(C)ncc3c2P(C)(C)=O)n1]

The title compound (22 mg, 27%) was prepared in a manner similar to that in Example 488. ¹H NMR (500 MHz, DMSO) δ 12.33 (s, 1H), 10.84 (s, 1H), 8.64 (s, 1H), 8.52 (s, 1H), 8.24 (s, 1H), 8.18 (s, 2H), 7.30 (s, 1H), 6.71 (s, 1H), 6.23 (d, J=12.2 Hz, 2H), 4.03-4.01 (m, 1H), 3.76-3.73 (m, 6H), 3.61-3.41 (m, 7H), 3.29-3.19 (m, 4H), 3.09-3.07 (m, 2H), 2.87-2.71 (m, 1H), 2.62-2.59 (m, 5H), 2.43-2.41 (m, 1H), 2.18-2.15 (m, 1H), 2.12-2.01 (m, 10H), 2.01-1.90 (m, 2H), 1.88-1.85 (d, 2H), 1.68-1.52 (m, 2H); [M+H]⁺=1030.5.

### Example 528: (R)-3-(4-((R)-3-(4-(1-(4-((5-bromo-4-((5-(dimethylphosphoryl)-2-methyl-1-oxo-1,2-dihydrophthalazin-6-yl)amino)pyrimidin-2-yl)amino)-2-ethyl-5-methoxyphenyl)piperidin-1)piperazine-1-carbonyl)pyrrolidin-1-yl)_{2,6}-difluorophenyl)piperidine-2,6-dione

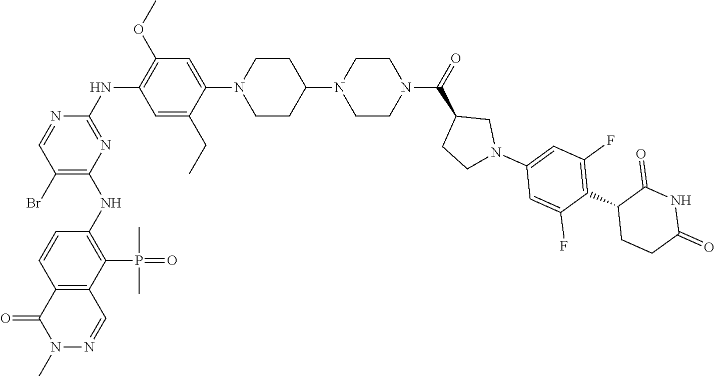 [SMILES: CCc1cc(Nc2ncc(Br)c(Nc3ccc4c(=O)n(C)ncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound (15 mg, 29%) was prepared in a manner similar to that in Example 488. ¹H NMR (500 MHz, DMSO) δ 12.21 (s, 1H), 10.84 (s, 1H), 8.56 (s, 2H), 8.25 (s, 1H), 8.21-8.14 (m, 2H), 7.30 (s, 1H), 6.77 (s, 1H), 6.24 (s, 1H), 6.22 (s, 1H), 4.02 (dd, J=12.6, 4.9 Hz, 1H), 3.75 (s, 3H), 3.72 (s, 3H), 3.60-3.41 (m, 8H), 3.01 (d, J=9.6 Hz, 2H), 2.82-2.68 (m, 4H), 2.58 (s, 2H), 2.45-2.36 (m, 4H), 2.04 (d, J=13.3 Hz, 11H), 1.97-1.92 (m, 1H), 1.85 (d, J=11.3 Hz, 2H), 1.61 (d, J=10.2 Hz, 2H), 0.94 (s, 3H). [M+H]⁺=1044.7

### Example 529: (R)-3-(4-((R)-3-(4-(1-(4-((5-bromo-4-((5-(dimethylphosphoryl)-2-methyl-1-oxo-1,2-dihydrophthalazin-6-yl)amino)pyrimidin-2-yl)amino)-5-ethoxy-2-methylphenyl)piperidin-4-yl)piperazine-1-carbonyl)pyrrolidin-1-yl)-2,6-difluorophenyl)piperidine-2,6-dione

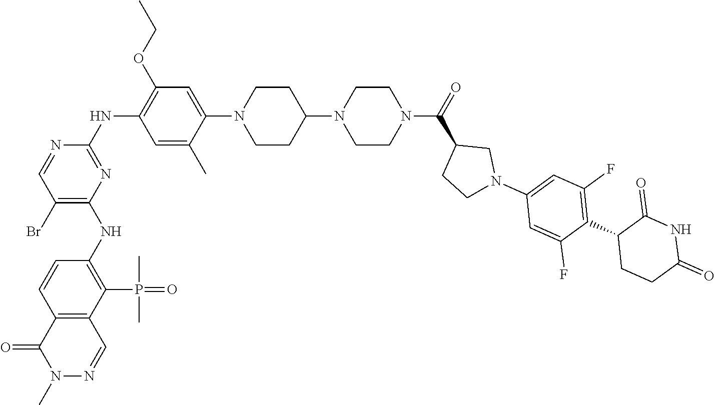 [SMILES: CCOc1cc(N2CCC(N3CCN(C(=O)[C@@H]4CCN(c5cc(F)c([C@H]6CCC(=O)NC6=O)c(F)c5)C4)CC3)CC2)c(C)cc1Nc1ncc(Br)c(Nc2ccc3c(=O)n(C)ncc3c2P(C)(C)=O)n1]

The title compound (20 mg, 40%) was prepared in a manner similar to that in Example 488. ¹H NMR (500 MHz, DMSO) δ 12.23 (s, 1H), 10.77 (s, 1H), 8.50 (s, 2H), 8.19 (s, 1H), 8.12 (s, 1H), 8.02 (s, 1H), 7.28 (s, 1H), 6.62 (s, 1H), 6.17 (s, 1H), 6.15 (s, 1H), 3.94 (d, J=6.9 Hz, 3H), 3.65 (s, 3H), 3.54-3.34 (m, 7H), 3.21-3.16 (m, 4H), 3.00 (d, J=10.0 Hz, 2H), 2.76-2.66 (m, 1H), 2.57-2.51 (m, 4H), 2.33 (t, J=11.3 Hz, 2H), 2.14-1.84 (m, 13H), 1.78 (d, J=11.1 Hz, 2H), 1.54 (d, J=10.5 Hz, 2H), 1.19 (s, 3H). [M+H]⁺=1044.7

### Example 530: (R)-3-(4-((R)-3-(4-(1-(4-((5-bromo-4-((5-(dimethylphosphoryl)-2-methyl-1-oxo-1,2-dihydrophthalazin-6-yl)amino)pyrimidin-2-yl)amino)-5-ethoxy-2-ethylphenyl)piperidin-4-yl)piperazine-1-carbonyl)pyrrolidin-1-yl)-2,6-difluorophenyl)piperidine-2,6-dione

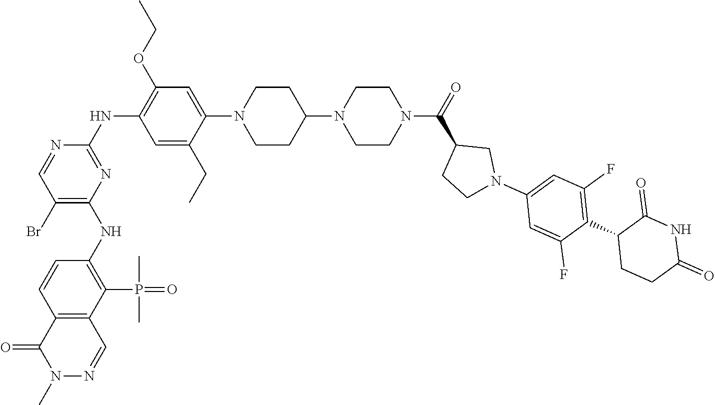 [SMILES: CCOc1cc(N2CCC(N3CCN(C(=O)[C@@H]4CCN(c5cc(F)c([C@H]6CCC(=O)NC6=O)c(F)c5)C4)CC3)CC2)c(CC)cc1Nc1ncc(Br)c(Nc2ccc3c(=O)n(C)ncc3c2P(C)(C)=O)n1]

The title compound (25 mg, 38%) was prepared in a manner similar to that in Example 488. ¹H NMR (500 MHz, DMSO) δ 12.08 (s, 1H), 10.78 (s, 1H), 8.50 (s, 2H), 8.15-8.06 (m, 2H), 8.03 (s, 1H), 7.30 (s, 1H), 6.67 (s, 1H), 6.17 (s, 1H), 6.15 (s, 1H), 4.04-3.85 (m, 4H), 3.55-3.33 (m, 9H), 3.20-3.15 (m, 2H), 2.91 (d, J=10.1 Hz, 2H), 2.70 (d, J=12.6 Hz, 1H), 2.59 (t, J=11.1 Hz, 2H), 2.51 (s, 2H), 2.33 (d, J=7.4 Hz, 4H), 2.15-1.84 (m, 12H), 1.77 (d, J=10.6 Hz, 2H), 1.53 (d, J=10.1 Hz, 2H), 1.18 (t, J=6.9 Hz, 3H), 0.82 (s, 3H). [M+H]⁺=1058.4.

### Example 531: (R)-3-(4-((R)-3-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazine-1-carbonyl)pyrrolidin-1-yl)-2,6-difluorophenyl)piperidine-2,6-dione

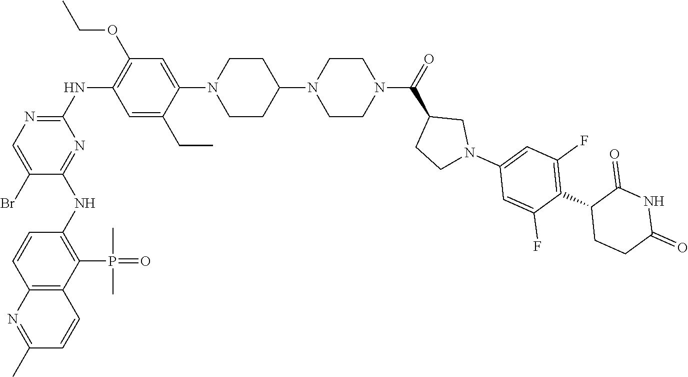 [SMILES: CCOc1cc(N2CCC(N3CCN(C(=O)[C@@H]4CCN(c5cc(F)c([C@H]6CCC(=O)NC6=O)c(F)c5)C4)CC3)CC2)c(CC)cc1Nc1ncc(Br)c(Nc2ccc3nc(C)ccc3c2P(C)(C)=O)n1]

The title compound (40 mg, 55%) was prepared in a manner similar to that in Example 488. ¹H NMR (500 MHz, DMSO) δ 11.70 (s, 1H), 10.84 (s, 1H), 8.60 (d, J=8.9 Hz, 1H), 8.27 (s, 1H), 8.23 (s, 1H), 7.89 (d, J=3.5 Hz, 2H), 7.43 (d, J=8.8 Hz, 2H), 6.70 (s, 1H), 6.23 (d, J=12.1 Hz, 2H), 4.00 (d, J=7.0 Hz, 3H), 3.61-3.42 (m, 7H), 3.25 (dd, J=16.2, 8.7 Hz, 3H), 2.93 (d, J=11.0 Hz, 2H), 2.84-2.70 (m, 1H), 2.64 (d, J=10.3 Hz, 8H), 2.37 (s, 1H), 2.26 (d, J=7.0 Hz, 2H), 2.20-2.04 (m, 4H), 1.98 (d, J=13.3 Hz, 7H), 1.82 (d, J=11.2 Hz, 2H), 1.62-1.49 (m, 2H), 1.26 (t, J=6.9 Hz, 3H), 0.71 (s, 3H). [M+H]⁺=1041.3.

### Example 532: 3-(6-(2-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H-yl)piperidine-2,6-dione

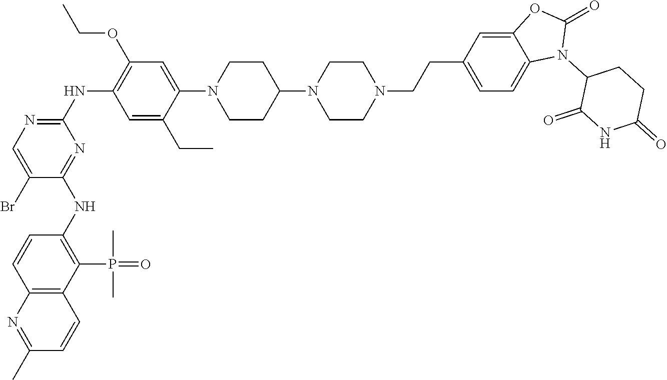 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2ccc3nc(C)ccc3c2P(C)(C)=O)n1]

The title compound (50 mg, 50%) was prepared in a manner similar to that in Example 492. [M+H]⁺=993.3. ¹H NMR (500 MHz, DMSO) δ 11.70 (s, 1H), 11.20 (s, 1H), 8.60 (d, J=8.8 Hz, 1H), 8.22 (s, 2H), 7.93-7.81 (m, 2H), 7.48-7.39 (m, 2H), 7.30 (s, 1H), 7.16 (d, J=8.1 Hz, 1H), 7.07 (d, J=8.5 Hz, 1H), 6.70 (s, 1H), 5.35 (s, 1H), 4.00 (q, J=7.0 Hz, 2H), 3.45 (s, 1H), 2.91 (d, J=11.2 Hz, 3H), 2.77 (t, J=7.5 Hz, 2H), 2.72-2.57 (m, 8H), 2.53 (d, J=9.7 Hz, 4H), 2.36 (s, 4H), 2.27 (d, J=9.1 Hz, 3H), 2.16 (dd, J=10.8, 5.2 Hz, 1H), 1.98 (d, J=13.3 Hz, 6H), 1.82 (d, J=11.9 Hz, 2H), 1.53 (d, J=8.8 Hz, 2H), 1.26 (t, J=6.9 Hz, 3H), 0.71 (s, 3H).

### Example 533: (R)-3-(4-((R)-3-(4-(1-(4-((5-bromo-4-((5-(dimethylphosphoryl)-2-ethyl-8-fluoroquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

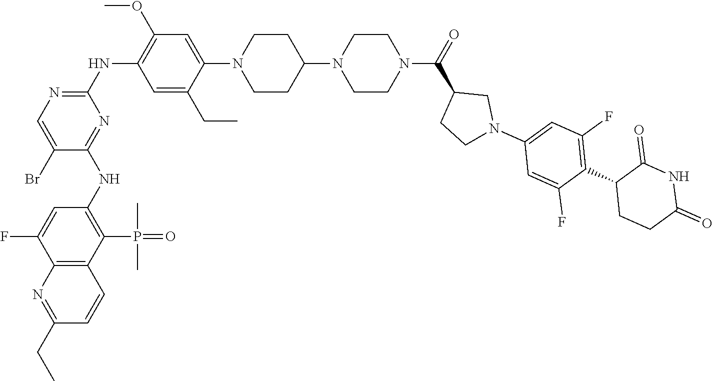 [SMILES: CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)cc(F)c2n1]

The title compound was prepared in the same procedure as Example 488. ¹H NMR (500 MHz, DMSO) δ 12.14 (s, 1H), 10.84 (s, 1H), 8.54 (d, J=9.0 Hz, 1H), 8.32-8.16 (m, 3H), 7.53 (d, J=9.0 Hz, 1H), 7.32 (s, 1H), 6.75 (s, 1H), 6.23 (d, J=12.2 Hz, 2H), 4.02 (dd, J=12.5, 4.9 Hz, 1H), 3.77 (s, 3H), 3.62-3.43 (m, 6H), 3.35-3.34 (m, 3H), 3.28-3.23 (m, 2H), 3.02-2.90 (m, 4H), 2.84-2.70 (m, 1H), 2.70-2.52 (m, 5H), 2.40-2.33 (m, 3H), 2.22-1.90 (m, 10H), 1.85-1.83 (m, 2H), 1.60-1.54 (m, 2H), 1.32 (t, J=7.6 Hz, 3H), 0.80 (s, 3H). [M+H]⁺=1059.6.

### Example 534: (R)-3-(4-(2-(4-(1-(4-((5-bromo-4-((5-(dimethylphosphoryl)-8-fluoro-2-methylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

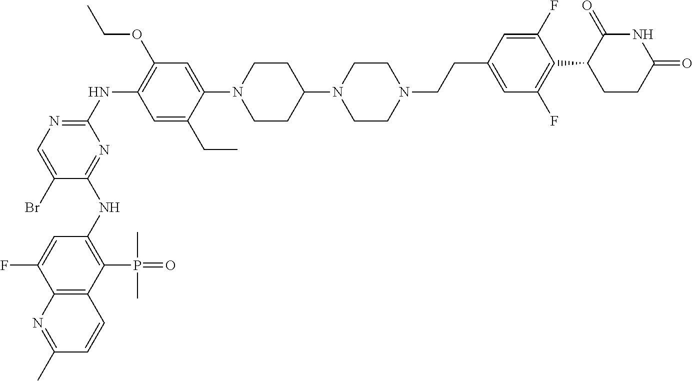 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2cc(F)c3nc(C)ccc3c2P(C)(C)=O)n1]

The title compound was prepared in a manner similar to that in Example 484. ¹H NMR (500 MHz, DMSO) δ 12.12 (s, 1H), 10.94 (s, 1H), 8.56-8.54 (m, 1H), 8.39-8.36 (m, 1H), 8.24-8.18 (m, 1H), 8.03 (s, 1H), 7.51 (d, J=9.0 Hz, 1H), 7.42 (s, 1H), 7.02 (d, J=10.1 Hz, 2H), 6.71 (s, 1H), 4.20 (dd, J=12.7, 5.0 Hz, 1H), 4.01 (q, J=6.9 Hz, 2H), 2.95-2.93 (m, 2H), 2.86-2.72 (m, 3H), 2.70-2.52 (m, 12H), 2.48-2.22 (m, 7H), 2.13-2.09 (m, 1H), 2.04-1.95 (m, 7H), 1.84-1.82 (m, 2H), 1.56-1.50 (m, 2H), 1.26 (t, J=6.9 Hz, 3H), 0.76 (s, 3H). [M+H]⁺=990.6.

### Example 535: 3-(4-(3-((4-(1-(4-((5-bromo-4-((5-(dimethylphosphoryl)-2-methyl-1-oxo-1,2-dihydrophthalazin-6-yl)amino)pyrimidin-2-yl)amino)-5-ethoxy-2-ethylphenyl)piperidin-4-yl)piperazin-1-yl)methyl)azetidin-1-yl)-2,6-difluorophenyl)piperidine-2,6-dione

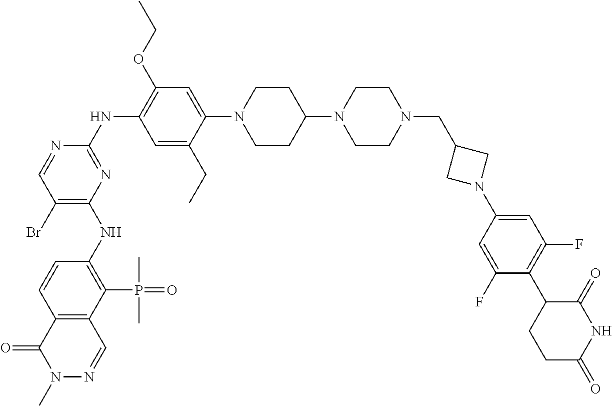 [SMILES: CCOc1cc(N2CCC(N3CCN(CC4CN(c5cc(F)c(C6CCC(=O)NC6=O)c(F)c5)C4)CC3)CC2)c(CC)cc1Nc1ncc(Br)c(Nc2ccc3c(=O)n(C)ncc3c2P(C)(C)=O)n1]

The title compound (10 mg, 32%) was prepared in a manner similar to that in Example 484. ¹H NMR (500 MHz, DMSO) δ 12.08 (s, 1H), 10.78 (s, 1H), 8.50 (s, 2H), 8.18 (s, 2H), 8.11 (s, 1H), 8.09 (s, 1H), 8.02 (s, 1H), 7.29 (s, 1H), 6.66 (s, 1H), 4.00-3.90 (m, 4H), 3.87 (d, J=7.5 Hz, 3H), 3.66 (s, 3H), 3.40 (t, J=6.0 Hz, 3H), 2.95-2.80 (m, 4H), 2.76-2.65 (m, 2H), 2.58 (t, J=11.0 Hz, 3H), 2.33 (s, 5H), 2.22 (t, J=11.0 Hz, 2H), 2.06-1.83 (m, 10H), 1.77 (d, J=10.7 Hz, 2H), 1.48 (d, J=11.1 Hz, 2H), 1.18 (t, J=6.9 Hz, 3H), 0.82 (s, 3H). [M+H]⁺=1030.4

### Example 536: 3-(6-(2-(4-(1-(4-((5-bromo-4-((5-(dimethylphosphoryl)-8-fluoro-2-methylquinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

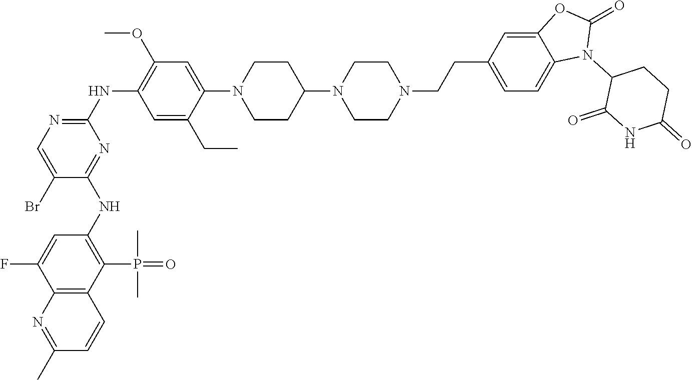 [SMILES: CCc1cc(Nc2ncc(Br)c(Nc3cc(F)c4nc(C)ccc4c3P(C)(C)=O)n2)c(OC)cc1N1CCC(N2CCN(CCc3ccc4c(c3)oc(=O)n4C3CCC(=O)NC3=O)CC2)CC1]

The title compound was prepared in a manner similar to that in Example 492. ¹H NMR (500 MHz, DMSO) δ 12.16 (s, 1H), 11.20 (s, 1H), 8.53 (d, J=6.7 Hz, 1H), 8.40-8.37 (m, 1H), 8.22 (s, 1H), 8.14 (s, 1H), 7.51 (d, J=9.0 Hz, 1H), 7.35 (s, 1H), 7.30 (s, 1H), 7.16 (d, J=8.1 Hz, 1H), 7.08 (d, J=8.1 Hz, 1H), 6.74 (s, 1H), 5.35 (dd, J=13.0, 5.3 Hz, 1H), 3.76 (s, 3H), 3.01-2.83 (m, 4H), 2.80-2.52 (m, 14H), 2.47-2.22 (m, 6H), 2.19-2.09 (m, 1H), 2.00 (d, J=13.3 Hz, 7H), 1.88-1.83 (m, 2H), 1.57-1.51 (m, 2H), 0.81 (s, 3H). [M+H]⁺=997.5.

### Example 537: 3-(6-(3-((4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)methyl)azetidin-1-yl)-2-oxobenzo[d]oxazol-3(2H)-yl)piperidine-2,6-dione

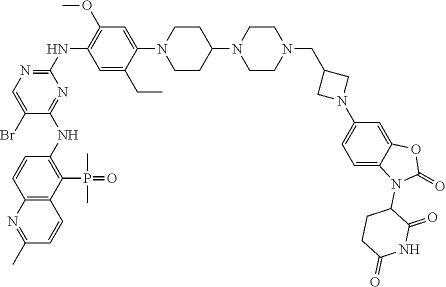 [SMILES: CCc1cc(Nc2ncc(Br)c(Nc3ccc4nc(C)ccc4c3P(C)(C)=O)n2)c(OC)cc1N1CCC(N2CCN(CC3CN(c4ccc5c(c4)oc(=O)n5C4CCC(=O)NC4=O)C3)CC2)CC1]

The title compound (4.91 mg, 7.56%) was prepared in a manner similar to that in Example 492. ¹H NMR (500 MHz, DMSO) δ 11.78 (s, 1H), 11.18 (s, 1H), 8.56 (d, J=8.8 Hz, 1H), 8.29 (s, 1H), 8.21 (s, 1H), 8.00 (s, 1H), 7.87 (d, J=9.3 Hz, 1H), 7.42 (d, J=8.9 Hz, 1H), 7.37 (s, 1H), 7.05 (d, J=8.4 Hz, 1H), 6.74 (s, 1H), 6.53 (s, 1H), 6.23 (d, J=8.7 Hz, 1H), 5.30-5.25 (m, 1H), 3.92-3.89 (m, 2H), 3.76 (s, 3H), 3.47-3.39 (m, 4H), 2.98-2.82 (m, 5H), 2.73-2.63 (m, 7H), 2.56-2.53 (m, 4H), 2.42-2.39 (m, 3H), 2.30-2.28 (m, 3H), 2.13-2.10 (m, 1H), 2.00 (s, 3H), 1.97 (s, 3H), 1.83 (d, J=11.3 Hz, 2H), 1.56-1.52 (m, 2H), 0.77 (s, 3H). [M+H]⁺=1020.5.

### Example 538: (R)-3-(4-((R)-3-(4-(1-(4-((5-bromo-4-((5-(dimethylphosphoryl)-2-ethylquinoxalin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

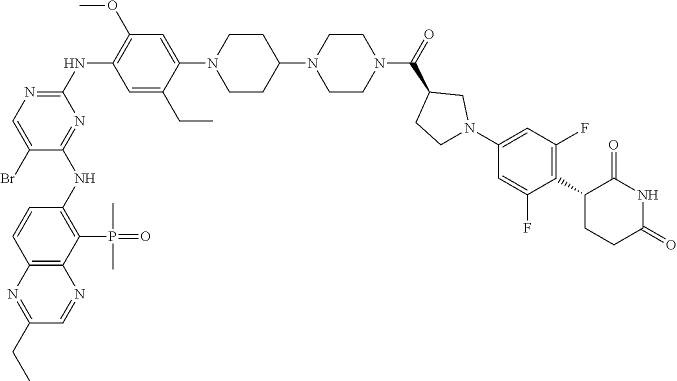 [SMILES: CCc1cnc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1]

To a solution of (6-((5-bromo-2-((5-ethyl-2-methoxy-4-(4-(piperazin-1-yl)piperidin-1-yl)phenyl)amino)pyrimidin-4-yl)amino)-2-ethylquinoxalin-5-yl)dimethylphosphine oxide (50 mg, 0.069 mmol, the compound was obtained by the way similar to example 484), (R)-1-(4-((R)-2,6-dioxopiperidin-3-yl)-3,5-difluorophenyl)pyrrolidine-3-carboxylic acid (35 mg, 0.10 mmol) and DIEA (45 mg, 0.35 mmol) in DMF (2 mL) was added HATU (38 mg, 0.10 mmol) at room temperature. The resulting mixture was stirred at room temperature for 1 h. The reaction was purified with prep-HPLC chromatography (0.1% FA in water: acetonitrile=90:10˜ 50:50 gradient elution) to give the product (9.8 mg, 13%).

¹H NMR (500 MHz, DMSO) δ 12.68 (s, 1H), 10.85 (s, 1H), 8.85 (s, 1H), 8.73 (s, 1H), 8.48 (s, 1H), 8.30 (d, J=5.7 Hz, 1H), 7.88 (d, J=8.6 Hz, 1H), 7.42 (s, 1H), 6.83 (s, 1H), 6.25 (d, J=12.1 Hz, 2H), 4.54 (d, J=13.3 Hz, 1H), 4.26 (d, J=13.3 Hz, 2H), 4.04-3.99 (m, 3H), 3.79 (s, 3H), 3.59 (d, J=6.9 Hz, 4H), 3.49 (s, 1H), 3.39-3.27 (m, 4H), 3.12 (d, J=8.6 Hz, 3H), 3.00 (d, J=7.6 Hz, 3H), 2.83-2.74 (m, 3H), 2.19 (s, 5H), 2.02 (d, J=14.4 Hz, 7H), 1.98-1.85 (m, 3H), 1.35 (t, J=7.6 Hz, 3H), 0.94 (s, 3H); [M+H]⁺=1042.6.

### Example 539: (R)-3-(4-((R)-3-(4-(1-(4-((5-bromo-4-((5-(dimethylphosphoryl)-2-ethylquinolin-6-yl)amino)pyrimidin-2-yl)amino)-5-methoxy-2-methylphenyl)piperidin-4-yl)piperazine-1-carbonyl)pyrrolidin-1-yl)-2, 6-difluorophenyl)piperidine-2,6-dione

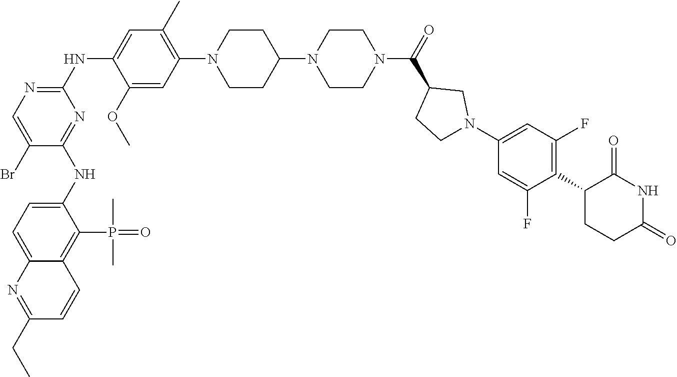 [SMILES: CCc1ccc2c(P(C)(C)=O)c(Nc3nc(Nc4cc(C)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1]

The title compound (20 mg, 35.0%) was prepared in a manner similar to that in Example 488. ¹H NMR (500 MHz, DMSO) δ 11.89 (s, 1H), 10.84 (s, 1H), 8.54 (d, J=8.5 Hz, 1H), 8.34 (s, 1H), 8.20 (s, 1H), 8.00 (s, 1H), 7.89 (d, J=9.4 Hz, 1H), 7.44 (d, J=8.9 Hz, 1H), 7.29 (s, 1H), 6.70 (s, 1H), 6.23 (d, J=12.2 Hz, 2H), 4.02 (dd, J=12.4, 4.7 Hz, 1H), 3.76 (s, 3H), 3.58-3.43 (m, 8H), 3.28-3.20 (m, 2H), 3.04 (d, J=10.1 Hz, 2H), 2.92 (q, J=7.5 Hz, 2H), 2.83-2.73 (m, 1H), 2.64 (t, J=11.0 Hz, 2H), 2.58-2.55 (m, 4H), 2.38-2.35 (m, 1H), 2.23-2.03 (m, 4H), 1.99 (d, J=13.3 Hz, 6H), 1.91 (s, 3H), 1.85 (d, J=10.5 Hz, 2H), 1.59 (d, J=10.8 Hz, 2H), 1.32 (t, J=7.6 Hz, 3H). [M+H]⁺=1027.7

### Example 541: (R)-3-(4-(3-((4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)methyl)azetidin-1-yl)2,6-difluorophenyl)piperidine-2,6-dione

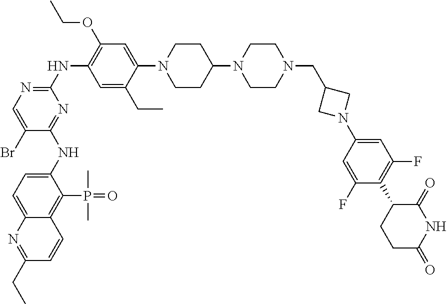 [SMILES: CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3nc(CC)ccc3c2P(C)(C)=O)n1]

The title compound (34 mg, 27.5%) was prepared in a manner similar to that in Example 484. ¹H NMR (500 MHz, DMSO) δ 11.73 (s, 1H), 10.85 (s, 1H), 8.60 (d, J=8.8 Hz, 1H), 8.25 (s, 1H), 8.22 (s, 1H), 7.91 (s, 1H), 7.87 (d, J=9.4 Hz, 1H), 7.45 (d, J=8.9 Hz, 1H), 7.38 (s, 1H), 6.71 (s, 1H), 6.10 (d, J=11.1 Hz, 2H), 4.06-3.97 (m, 3H), 3.93 (t, J=7.5 Hz, 2H), 3.47 (t, J=5.9 Hz, 2H), 3.30-3.28 (m, 2H), 2.96-2.93 (m, 5H), 2.82-2.73 (m, 1H), 2.63 (t, J=11.2 Hz, 2H), 2.56-2.51 (m, 6H), 2.41-2.38 (m, 3H), 2.29-2.25 (m, 3H), 2.07 (dt, J=12.7, 9.2 Hz, 1H), 1.98 (d, J=13.3 Hz, 6H), 1.96-1.89 (m, 1H), 1.82 (d, J=11.8 Hz, 2H), 1.52 (dd, J=20.4, 11.3 Hz, 2H), 1.32 (t, J=7.6 Hz, 3H), 1.25 (t, J=6.9 Hz, 3H), 0.71 (s, 3H). [M+H]⁺=1027.7

### Example 542: 3-(4-((5-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecan-3-yl)pentyl)oxy)-1-oxoisoindolin-2-yl)piperidine-2,6-dione

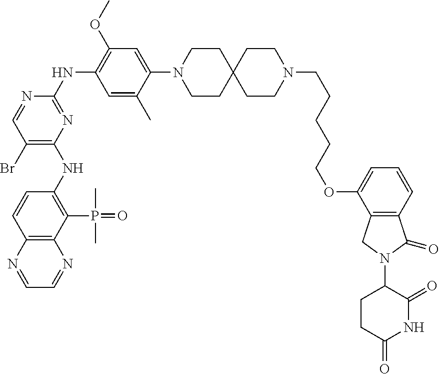 [SMILES: COc1cc(N2CCC3(CCN(CCCCCOc4cccc5c4CN(C4CCC(=O)NC4=O)C5=O)CC3)CC2)c(C)cc1Nc1ncc(Br)c(Nc2ccc3nccnc3c2P(C)(C)=O)n1]

The title compound (55 mg, 54%) was prepared according to the procedure shown in WO2021036922A. ¹H NMR (500 MHz, DMSO) δ 12.68 (s, 1H), 10.98 (s, 1H), 8.87 (d, J=1.8 Hz, 1H), 8.84 (d, J=1.8 Hz, 1H), 8.26 (s, 2H), 8.24 (s, 1H), 7.92 (d, J=9.1 Hz, 1H), 7.48 (t, J=7.8 Hz, 1H), 7.35-7.29 (m, 2H), 7.25 (d, J=8.1 Hz, 1H), 6.81 (s, 1H), 5.12 (dd, J=13.3, 5.1 Hz, 1H), 4.38 (d, J=17.3 Hz, 1H), 4.23 (d, J=17.3 Hz, 1H), 4.13 (t, J=6.3 Hz, 2H), 3.78 (s, 3H), 2.97-2.87 (m, 1H), 2.81 (s, 4H), 2.59 (d, J=17.8 Hz, 3H), 2.44 (dd, J=13.1, 4.5 Hz, 4H), 2.08 (s, 3H), 2.06-2.01 (m, 8H), 1.83-1.71 (m, 2H), 1.59-1.56 (m, 10H), 1.49-1.41 (m, 2H). [M+H]⁺=993.4.

### Example 544: (R)-3-(4-((R)-3-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazine-1-carbonyl)pyrrolidin-1-yl)-2,6-difluorophenyl)piperidine-2,6-dione

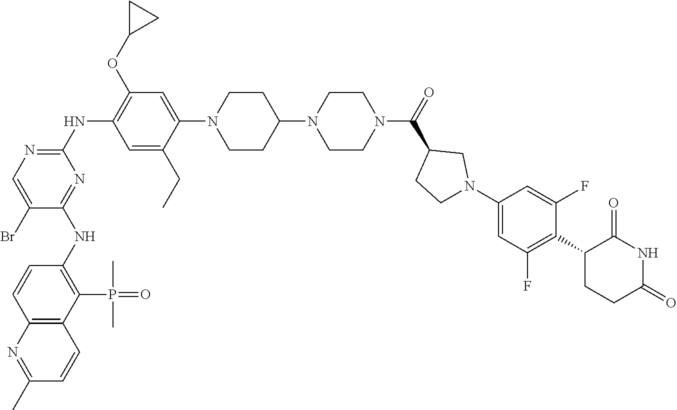 [SMILES: CCc1cc(Nc2ncc(Br)c(Nc3ccc4nc(C)ccc4c3P(C)(C)=O)n2)c(OC2CC2)cc1N1CCC(N2CCN(C(=O)[C@@H]3CCN(c4cc(F)c([C@H]5CCC(=O)NC5=O)c(F)c4)C3)CC2)CC1]

The title compound (7 mg, 10%) was prepared in a manner similar to that in Example 488. ¹H NMR (500 MHz, DMSO) δ 11.77 (s, 1H), 10.84 (s, 1H), 8.58 (d, J=8.5 Hz, 1H), 8.25 (s, 1H), 8.20 (s, 1H), 7.94-7.79 (m, 2H), 7.45 (d, J=8.8 Hz, 1H), 7.35 (s, 1H), 6.99 (s, 1H), 6.23 (d, J=12.2 Hz, 2H), 4.02 (dd, J=12.6, 4.8 Hz, 1H), 3.83-3.79 (m, 1H), 3.61-3.43 (m, 8H), 3.28-3.22 (m, 3H), 3.01-2.94 (m, 4H), 2.83-2.74 (m, 1H), 2.70-2.53 (m, 5H), 2.43-2.55 (m, 3H), 2.21-1.92 (m, 8H), 1.87-1.83 (m, 2H), 1.58 (dd, J=20.4, 10.7 Hz, 2H), 1.32 (t, J=7.6 Hz, 3H), 0.83-0.67 (m, 5H), 0.61-0.55 (m, 2H). [M+H]⁺=1053.3.

### Example 547: (R)-3-(4-(2-(4-(1-(4-((5-bromo-4-((5-(dimethylphosphoryl)-2-ethylquinolin-6-yl)amino)pyrimidin-2-yl)amino)-5-cyclopropoxy-2-ethylphenyl)piperidin-4-yl)piperazin-1-yl)ethyl)-2,6-difluorophenyl)piperidine-2,6-dione

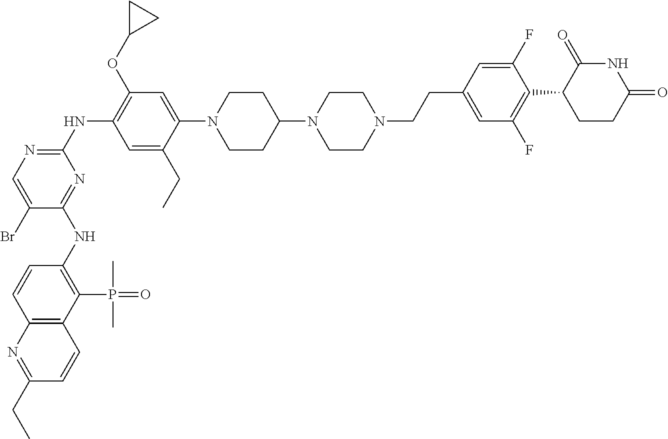 [SMILES: CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC4CC4)ncc3Br)ccc2n1]

The title compound (7 mg, 10%) was prepared in a manner similar to that in Example 484. ¹H NMR (500 MHz, DMSO) δ 11.76 (s, 1H), 10.95 (s, 1H), 8.58 (d, J=8.9 Hz, 1H), 8.25 (d, J=5.0 Hz, 1H), 8.20 (s, 1H), 7.90-7.81 (m, 2H), 7.45 (d, J=8.9 Hz, 1H), 7.34 (s, 1H), 7.03 (d, J=10.0 Hz, 2H), 6.99 (s, 1H), 4.20 (dd, J=12.6, 5.0 Hz, 1H), 3.81 (ddd, J=8.9, 5.9, 2.9 Hz, 1H), 2.98-2.90 (m, 4H), 2.85-2.74 (m, 3H), 2.69-2.52 (m, 11H), 2.47-2.21 (m, 5H), 2.13 (qd, J=13.0, 3.8 Hz, 1H), 2.01-1.97 (m, 7H), 1.86 (d, J=10.8 Hz, 2H), 1.61-1.52 (m, 2H), 1.32 (t, J=7.6 Hz, 3H), 0.83-0.67 (m, 5H), 0.61-0.55 (m, 2H). [M+H]⁺=998.7.

### Example 548: 3-(6-(2-(4-(1-(4-((5-bromo-4-((5-(dimethylphosphoryl)-2-ethylquinoxalin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

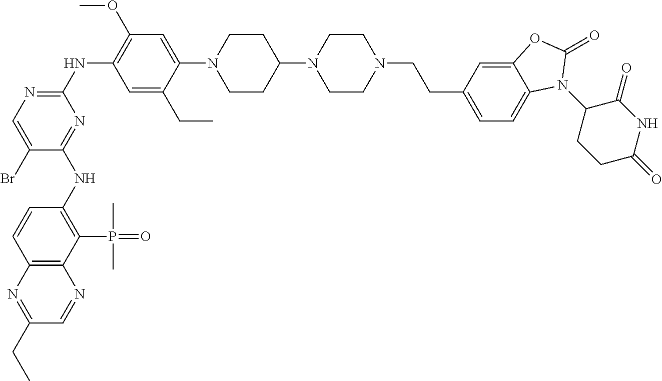 [SMILES: CCc1cnc2c(P(C)(C)=O)c(Nc3nc(Nc4cc(CC)c(N5CCC(N6CCN(CCc7ccc8c(c7)oc(=O)n8C7CCC(=O)NC7=O)CC6)CC5)cc4OC)ncc3Br)ccc2n1]

The title compound was synthesized in a manner similar to that in Example 492. ¹H NMR (500 MHz, DMSO) δ 12.44 (s, 1H), 11.14 (s, 1H), 8.75 (s, 1H), 8.63 (s, 1H), 8.17 (t, J=16.1 Hz, 2H), 7.77 (d, J=9.7 Hz, 1H), 7.27 (s, 1H), 7.23 (s, 1H), 7.09 (d, J=7.8 Hz, 1H), 7.01 (d, J=7.9 Hz, 1H), 6.74 (s, 1H), 5.33-5.22 (m, 1H), 3.69 (s, 3H), 2.98-2.88 (m, 4H), 2.82 (s, 1H), 2.74-2.54 (m, 7H), 2.47 (s, 6H), 2.41 (s, 5H), 2.25 (s, 1H), 2.09 (d, J=7.4 Hz, 1H), 1.94 (d, J=14.3 Hz, 6H), 1.79 (d, J=10.1 Hz, 2H), 1.52 (d, J=10.3 Hz, 2H), 1.27 (t, J=7.5 Hz, 3H), 0.87 (s, 3H); [M+H]⁺=994.6.

### Example 549: (R)-3-(4-((R)-3-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

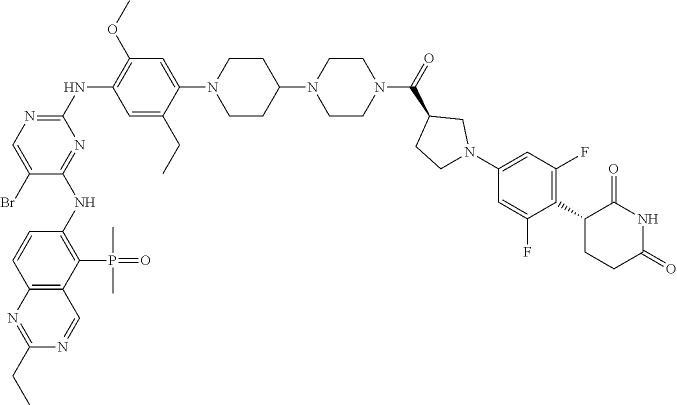 [SMILES: CCc1ncc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1]

The title compound (26 mg, 56%) was prepared in a manner similar to that in Example 488. ¹H NMR (500 MHz, DMSO) δ 11.60 (s, 1H), 10.84 (s, 1H), 9.86 (s, 1H), 8.46 (s, 1H), 8.24 (s, 1H), 8.05 (s, 1H), 7.86 (d, J=9.1 Hz, 1H), 7.28 (s, 1H), 6.75 (s, 1H), 6.23 (d, J=12.2 Hz, 2H), 4.02 (dd, J=12.4, 4.9 Hz, 1H), 3.75 (s, 3H), 3.62-3.55 (m, 4H), 3.53-3.48 (m, 3H), 3.33-3.29 (m, 2H), 3.27-3.22 (m, 1H), 3.08-3.03 (m, 2H), 2.95 (d, J=10.0 Hz, 2H), 2.77 (d, J=12.3 Hz, 1H), 2.68 (d, J=11.3 Hz, 2H), 2.57 (s, 3H), 2.38 (s, 1H), 2.27 (s, 2H), 2.17 (d, J=9.0 Hz, 1H), 2.08 (d, J=8.5 Hz, 2H), 2.03 (d, J=13.4 Hz, 7H), 1.97-1.92 (m, 1H), 1.84 (d, J=10.1 Hz, 2H), 1.57 (d, J=9.8 Hz, 2H), 1.38 (t, J=7.5 Hz, 3H), 0.75 (s, 3H); [M+H]⁺=1042.7.

### Example 550: (R)-3-(4-(2-(4-(1-(4-((5-bromo-4-((5-(dimethylphosphoryl)-2-ethylquinolin-6-yl)amino)pyrimidin-2-yl)amino)-2-cyclopropyl-5-methoxyphenyl)piperidin-4-yl)piperazin-1-yl)ethyl)-2,6-difluorophenyl)piperidine-2,6-dione

### Step 1: 1-cyclopropyl-2-fluoro-4-methoxy-5-nitrobenzene

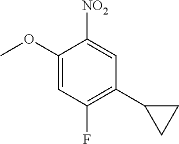

To a solution of 1-bromo-2-fluoro-4-methoxy-5-nitrobenzene (1 g, 4.0 mmol) and cyclopropylboronic acid (688 mg, 8.0 mmol) in dioxane/H₂O (20/5 mL) was added K₂CO₃ (1.1 g, 8.0 mmol) at 20° C. Pd(dppf)Cl₂ (303 mg, 0.4 mmol) was added to the mixture at 20° C. The flask was evacuated and backfilled with nitrogen three times. Then the mixture was stirred at 100° C. overnight. The mixture was filtered and concentrated in vacuum. The residue was purified by silica gel column chromatography (PE: EA=100: 0-1: 1 gradient elution) to give the desired product (600 mg, 71%). [M+H]⁺=212.1.

### Step 2: tert-butyl 4-(1-(2-cyclopropyl-5-methoxy-4-nitrophenyl)piperidin-4-yl)piperazine-1-carboxylate

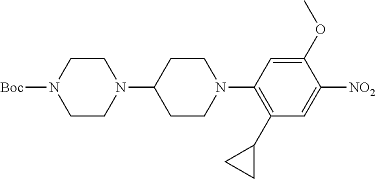 [SMILES: COc1cc(N2CCC(N3CCN(C(=O)OC(C)(C)C)CC3)CC2)c(C2CC2)cc1[N+](=O)[O-]]

To a solution of 1-cyclopropyl-2-fluoro-4-methoxy-5-nitrobenzene (600 mg, 2.8 mmol) and tert-butyl 4-(piperidin-4-yl)piperazine-1-carboxylate (915 mg, 3.4 mmol) in DMSO (20 mL) was added K₂CO₃ (773 mg, 5.6 mmol) at room temperature. The resulting mixture was stirred at 100° C. overnight. The reaction was diluted with water (50 mL) and extracted with DCM (3×20 mL). The combined organic layers were washed with brine (30 mL×3), dried over anhydrous Na₂SO₄, filtered and evaporated in vacuum to afford the crude residue, which was purified by silica gel column chromatography (DCM: MeOH=100: 0-10: 1 gradient elution) to give the desired product (800 mg, 66%). [M+H]⁺=461.2.

### Step 3: tert-butyl 4-(1-(4-amino-2-cyclopropyl-5-methoxyphenyl)piperidin-4-yl)piperazine-1-carboxylate

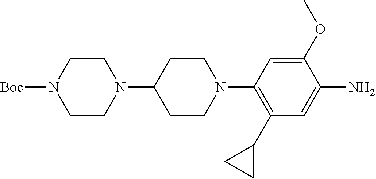 [SMILES: COc1cc(N2CCC(N3CCN(C(=O)OC(C)(C)C)CC3)CC2)c(C2CC2)cc1N]

To a solution of tert-butyl 4-(1-(2-cyclopropyl-5-methoxy-4-nitrophenyl)piperidin-4-yl)piperazine-1-carboxylate (800 mg, 1.9 mmol) in MeOH (20 mL) was added Pd/C (wet, 10 wt. %, 160 mg) at room temperature under hydrogen atmosphere. The resulting mixture was stirred at rt for 2 hours. The mixture was filtered and concentrated under reduced pressure to afford the desired product (700 mg, 90%). [M+H]⁺=431.2.

### Step 4: (6-((5-bromo-2-((5-cyclopropyl-2-methoxy-4-(4-(piperazin-1-yl)piperidin-1-yl)phenyl)amino)pyrimidin-4-yl)amino)-2-ethylquinolin-5-yl)dimethylphosphine oxide

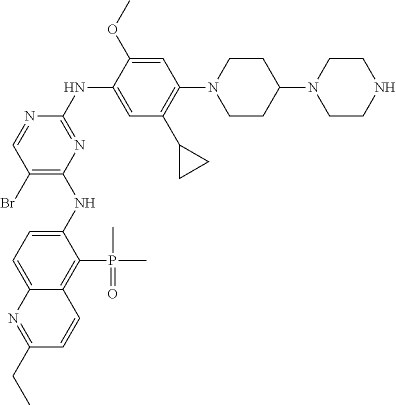 [SMILES: CCc1ccc2c(P(C)(C)=O)c(Nc3nc(Nc4cc(C5CC5)c(N5CCC(N6CCNCC6)CC5)cc4OC)ncc3Br)ccc2n1]

To a solution of (6-((5-bromo-2-chloropyrimidin-4-yl)amino)-2-ethylquinolin-5-yl)dimethylphosphine oxide (500 mg, 1.1 mmol) and tert-butyl 4-(1-(4-amino-2-cyclopropyl-5-methoxyphenyl)piperidin-4-yl)piperazine-1-carboxylate (473 mg, 1.1 mmol) in n-BuOH (20 mL) was added Ts-OH (580 g, 3.3 mmol) at room temperature. The resulting mixture was stirred at 100° C. overnight. The reaction was concentrated and basified with 0.5N NaOH (10 mL), then extracted with DCM (3×20 mL). The combined organic layers were washed with brine (30 mL×3), dried over anhydrous Na₂SO₄, filtered and evaporated in vacuum to afford the crude residue, which was purified by silica gel column chromatography (DCM: MeOH=100: 0-5: 1 gradient elution) to give the desired product (500 mg, 62%). [M+H]⁺=733.4.

### Step 5: (R)-3-(4-(2-(4-(1-(4-((5-bromo-4-((5-(dimethylphosphoryl)-2-ethylquinolin-6-yl)amino)pyrimidin-2-yl)amino)-2-cyclopropyl-5-methoxyphenyl)piperidin-4-yl)piperazin-1-yl)ethyl)-2,6-difluorophenyl)piperidine-2,6-dione

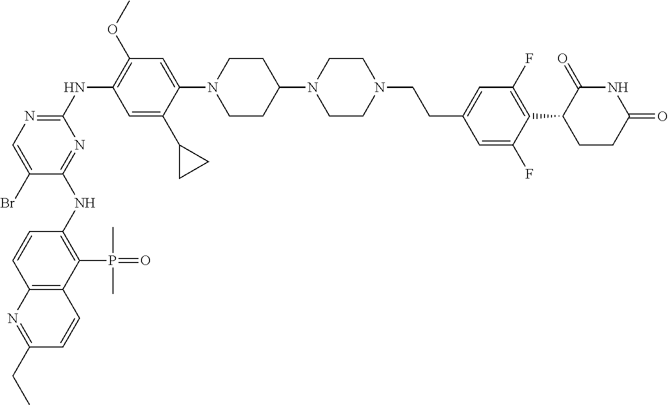 [SMILES: CCc1ccc2c(P(C)(C)=O)c(Nc3nc(Nc4cc(C5CC5)c(N5CCC(N6CCN(CCc7cc(F)c([C@H]8CCC(=O)NC8=O)c(F)c7)CC6)CC5)cc4OC)ncc3Br)ccc2n1]

To a solution of (6-((5-bromo-2-((5-cyclopropyl-2-methoxy-4-(4-(piperazin-1-yl)piperidin-1-yl)phenyl)amino)pyrimidin-4-yl)amino)-2-ethylquinolin-5-yl)dimethylphosphine oxide (50 mg, 0.07 mmol) and (R)-2-(4-(2,6-dioxopiperidin-3-yl)-3,5-difluorophenyl)acetaldehyde (22 mg, 0.08 mmol) in DCM (10 mL) was added NaBH(OAc)₃ (30 mg, 0.14 mmol) at room temperature. The resulting mixture was stirred at room temperature for 1 hour. The resulting mixture was diluted with H₂O (30 mL), and the layers were separated. The aqueous layer was extracted with DCM (3×15 mL). The combined organic layers were washed with brine (20 mL×3), dried over anhydrous Na₂SO₄, filtered and concentrated under reduced pressure, which was purified by silica gel column chromatography (DCM: MeOH=100: 0-10: 1 gradient elution) to give an impure product, which was further purified with prep-HPLC to give the desired product (12 mg, 17%). ¹H NMR (500 MHz, DMSO) δ 12.07 (s, 1H), 10.95 (s, 1H), 8.42 (d, J=8.7 Hz, 1H), 8.32 (s, 1H), 8.18 (s, 1H), 8.09 (s, 1H), 7.81 (d, J=9.4 Hz, 1H), 7.42 (d, J=8.9 Hz, 1H), 7.04 (s, 1H), 7.02 (s, 1H), 6.81 (s, 1H), 6.71 (s, 1H), 4.20 (dd, J=12.6, 4.9 Hz, 1H), 3.74 (s, 3H), 3.26 (d, J=10.8 Hz, 2H), 2.91 (d, J=7.6 Hz, 2H), 2.81-2.74 (m, 3H), 2.71-2.66 (m, 4H), 2.57-2.53 (m, 9H), 2.47-2.41 (m, 2H), 2.37-2.28 (m, 2H), 2.01-1.99 (m, 10H), 1.87 (d, J=11.1 Hz, 2H), 1.60 (d, J=11.3 Hz, 2H), 1.31 (t, J=7.6 Hz, 3H). [M+H]⁺=984.4.

### Example 551: (R)-3-(4-(3-((4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)methyl)azetidin-1-yl)-2,6-difluorophenyl)piperidine-2,6-dione

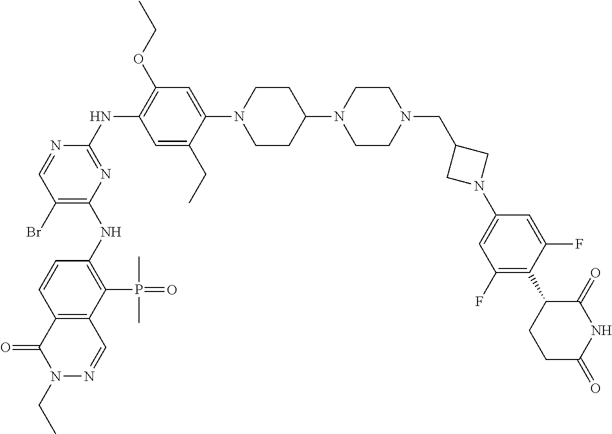 [SMILES: CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3c(=O)n(CC)ncc3c2P(C)(C)=O)n1]

The title compound (30 mg, 41%) was prepared in a manner similar to that in Example 484. ¹H NMR (500 MHz, DMSO) δ 12.11 (s, 1H), 10.85 (s, 1H), 8.64 (s, 1H), 8.52 (s, 1H), 8.26 (s, 1H), 8.18 (d, J=8.9 Hz, 1H), 8.09 (s, 1H), 7.34 (s, 1H), 6.73 (s, 1H), 6.12 (s, 1H), 6.09 (s, 1H), 4.17 (d, J=7.1 Hz, 2H), 4.00 (d, J=6.9 Hz, 3H), 3.93 (s, 2H), 3.47 (s, 3H), 3.00-2.86 (m, 3H), 2.82-2.73 (m, 1H), 2.64 (t, J=11.1 Hz, 2H), 2.55 (d, J=6.8 Hz, 6H), 2.39-2.34 (m, 8H), 2.03 (d, J=13.4 Hz, 6H), 1.96-1.90 (m, 2H), 1.83 (d, J=11.0 Hz, 2H), 1.55 (d, J=11.3 Hz, 2H), 1.31 (t, J=7.1 Hz, 3H), 1.25 (t, J=6.9 Hz, 3H), 0.87 (s, 3H). [M+H]⁺=1044.4.

### Example 552: 3-(6-(2-(4-(1-(4-((5-bromo-4-((5-(dimethylphosphoryl)-2-ethylquinolin-6-yl)amino)pyrimidin-2-yl)amino)-5-ethoxy-2-methylphenyl)piperidin-4-yl)piperazin-1-yl)ethyl)-2-oxobenzo[d]oxazol-3 (2H)-yl)piperidine-2,6-dione

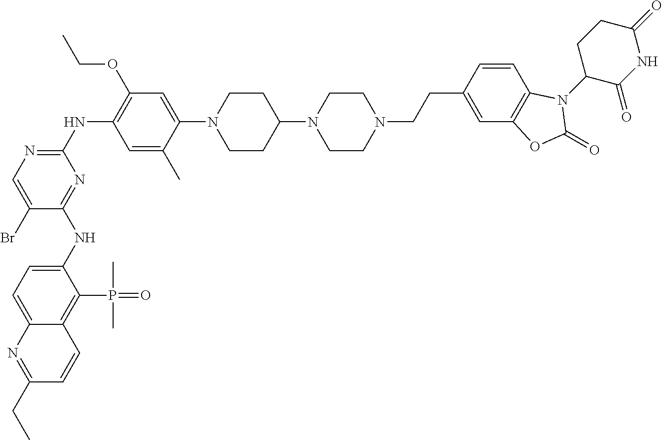 [SMILES: CCOc1cc(N2CCC(N3CCN(CCc4ccc5c(c4)oc(=O)n5C4CCC(=O)NC4=O)CC3)CC2)c(C)cc1Nc1ncc(Br)c(Nc2ccc3nc(CC)ccc3c2P(C)(C)=O)n1]

The title compound (14 mg, 24.4%) was prepared in a manner similar to that in Example 492. ¹H NMR (500 MHz, DMSO) δ 11.84 (s, 1H), 11.20 (s, 1H), 8.57 (d, J=9.1 Hz, 1H), 8.33 (s, 1H), 8.21 (s, 1H), 7.89 (d, J=9.4 Hz, 2H), 7.45 (d, J=9.0 Hz, 1H), 7.34 (s, 1H), 7.31 (s, 1H), 7.16 (d, J=8.0 Hz, 1H), 7.08 (d, J=7.6 Hz, 1H), 6.66 (s, 1H), 5.35 (dd, J=13.0, 5.4 Hz, 1H), 4.00 (q, J=6.9 Hz, 2H), 3.32 (m, 2H), 3.29 (s, 1H), 3.01 (d, J=10.9 Hz, 2H), 2.97-2.85 (m, 3H), 2.79-2.74 (m, 2H), 2.66-2.61 (m, 12H), 2.19-2.14 (m, 1H), 1.99 (d, J=13.3 Hz, 6H), 1.89-1.84 (m, 5H), 1.56 (s, 2H), 1.32 (t, J=7.6 Hz, 3H), 1.26 (t, J=6.9 Hz, 3H). [M+H]⁺=993.7

### Example 553: 3-(6-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-5-methyl-2-oxobenzo[d]oxazol-3(2H)-yl)piperidine-2,6-dione

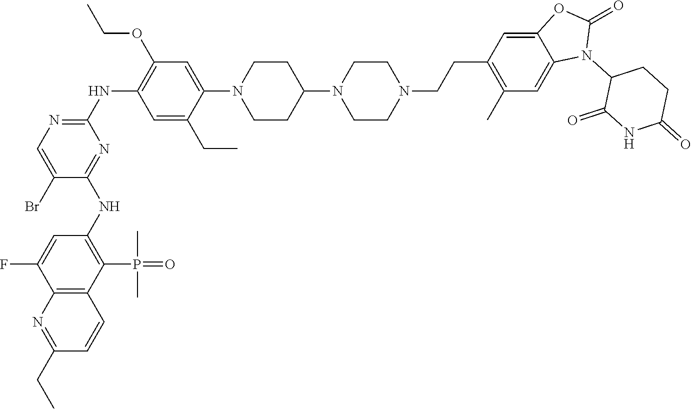 [SMILES: CCOc1cc(N2CCC(N3CCN(CCc4cc5oc(=O)n(C6CCC(=O)NC6=O)c5cc4C)CC3)CC2)c(CC)cc1Nc1ncc(Br)c(Nc2cc(F)c3nc(CC)ccc3c2P(C)(C)=O)n1]

The title compound was prepared in a manner similar to that in Example 492.

¹H NMR (500 MHz, DMSO) δ 12.03 (s, 1H), 11.12 (s, 1H), 8.51-8.50 (m, 1H), 8.24-8.17 (m, 2H), 7.97 (s, 1H), 7.47 (d, J=9.0 Hz, 1H), 7.33 (s, 1H), 7.15 (s, 1H), 7.02 (s, 1H), 6.64 (s, 1H), 5.25 (dd, J=13.0, 5.3 Hz, 1H), 3.94 (q, J=7.0 Hz, 2H), 2.95-2.76 (m, 6H), 2.72-2.45 (m, 10H), 2.41-2.13 (m, 11H), 2.10-2.01 (m, 1H), 1.93 (d, J=13.3 Hz, 6H), 1.77-1.75 (m, 2H), 1.49-1.43 (m, 2H), 1.25 (t, J=7.6 Hz, 3H), 1.19 (t, J=6.9 Hz, 3H), 0.68 (s, 3H). [M+H]⁺=1039.4.

### Example 554: 3-(6-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-5-methyl-2-oxobenzo[d]oxazol-3(2H)-yl)piperidine-2,6-dione

### Step 1: 5-methylbenzo[d]oxazol-2(3H)-one

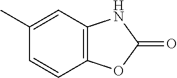

To a stirred solution of 2-amino-4-methylphenol (10.0 g, 81.2 mmol) in THF (100 mL) was added CDI (15.8 g, 97.4 mmol) at room temperature, then it was refluxed for 2 hrs. After cooled to rt, the reaction mixture was diluted with EA (200 mL). The organic layer was then washed with water (100 mL), brine (100 mL), dried over anhydrous Na₂SO₄, filtered and concentrated under reduced pressure to afford product (9.0 g, 74.3%), which was used without further purification. [M−H]⁻=148.0.

### Step 2: 6-bromo-5-methylbenzo[d]oxazol-2(3H)-one

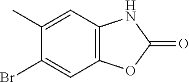

To a stirred solution of 5-methylbenzo[d]oxazol-2(3H)-one (9.0 g, 60.3 mmol) in AcOH (50 mL), was added NBS (12.9 g, 72.4 mmol) portionwise. The resulting mixture was stirred at rt overnight. The solvent was removed under reduced pressure, the residue was dissolved in EA (800 mL), and washed with water (200 mL), brine (200 mL), dried over Na₂SO₄, filtered, then concentrated. The residue was purified by column chromatography (DCM/MeOH, 20:1) to afford product (11.0 g, 80.0%). [M−H]⁻=226.0.

### Step 3: 3-(6-bromo-5-methyl-2-oxobenzo[d]oxazol-3(2H₁)-yl)piperidine-2,6-dione

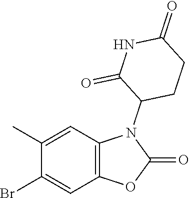 [SMILES: Cc1cc2c(cc1Br)oc(=O)n2C1CCC(=O)NC1=O]

A 50 mL three-neck round bottle flask was charged with 6-bromo-5-methylbenzo[d]oxazol-2(3H)-one (2.0 g, 8.8 mmol), dry DMF (10 mL), and Cs₂CO₃ (5.7 g, 17.5 mmol) at rt. The RBF was evacuated and backfilled with nitrogen three times. The resulting mixture was stirred at 70° C. for 1 hr. Then a solution of 3-bromopiperidine-2,6-dione (6.7 g, 35.1 mmol) in dry DMF (10 mL) was injected into the RBF by a syringe pump during a period of 2 hrs at 70° C. The resulting mixture was stirred overnight at 70° C. After cooled to rt, the mixture was diluted with EA (500 mL), washed with 1 N HCl (200 mL), water (3×200 mL), and brine (200 mL), then dried over Na₂SO₄, filtered, and concentrated. The residue was purified by column chromatography (PE/EA, 60%-30%) to afford product (1.2 g, 40.2%). [M−H]⁻=336.9.

### Step 4: (E)-3-(6)-2-ethoxyvinyl)-5-methyl-2-oxobenzo[d]oxazol-3(2H)-yl)piperidine-2,6-dione

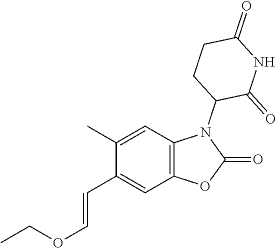 [SMILES: CCO/C=C/c1cc2oc(=O)n(C3CCC(=O)NC3=O)c2cc1C]

A 20 mL microwave vial were charged with 3-(6-bromo-5-methyl-2-oxobenzo[d]oxazol-3(2H)-yl)piperidine-2,6-dione (1.2 g, 3.5 mmol), CsF (797 mg, 5.2 mmol), Pd(dtbpf)Cl₂ (228 mg, 0.35 mmol), (E)-2-(2-ethoxyvinyl)-4,4,5,5-tetramethyl-1,3,2-dioxaborolane (1.0 g, 5.2 mmol), and DMF/H₂O (12 mL/4 mL). The vial was evacuated and backfilled with nitrogen three times. The resulting mixture was stirred at 50° C. for 1 hr. After cooled to rt, the mixture was diluted with EA (200 mL), washed with water (2×100 mL), brine (100 mL), dried over Na₂SO₄, filtered, and concentrated. The residue was purified by column chromatography (PE/EA, 60%-30%) to afford crude product, which is triturated with Et₂O to afford the target product (775 mg, 65.7%). [M+H]⁺=331.1.

### Step 5: 2-(3-(2,6-dioxopiperidin-3-yl)-5-methyl-2-oxo-2,3-dihydrobenzo[d]oxazol-6-yl)acetaldehyde

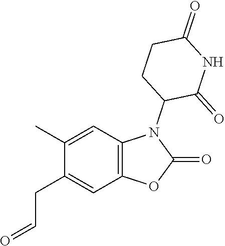 [SMILES: Cc1cc2c(cc1CC=O)oc(=O)n2C1CCC(=O)NC1=O]

The title compound (210 mg, 55%) was prepared in a manner similar to that in Example 506 step 3 from (E)-3-(6-(2-ethoxyvinyl)-5-methyl-2-oxobenzo[d]oxazol-3(2H)-yl)piperidine-2,6-dione and HCOOH.

### Step 6: 3-(6-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-1)piperazin-1-yl)ethyl)-5-methyl-2-oxobenzo[d]oxazol-3(2H)-yl)piperidine-2,6-dione

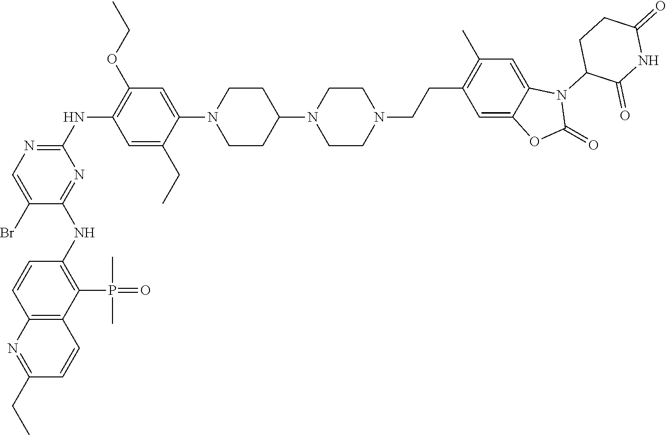 [SMILES: CCOc1cc(N2CCC(N3CCN(CCc4cc5oc(=O)n(C6CCC(=O)NC6=O)c5cc4C)CC3)CC2)c(CC)cc1Nc1ncc(Br)c(Nc2ccc3nc(CC)ccc3c2P(C)(C)=O)n1]

The title compound (40 mg, 45%) was prepared in a manner similar to that in Example 492 step 6 from 6-((5-bromo-2-((2-ethoxy-5-ethyl-4-(4-(piperazin-1-yl)piperidin-1-yl)phenyl)amino)pyrimidin-4-yl)amino)-5-(dimethylphosphoryl)-2-ethylphthalazin-1(2H)-one and 2-(3-(2,6-dioxopiperidin-3-yl)-5-methyl-2-oxo-2,3-dihydrobenzo[d]oxazol-6-yl)acetaldehyde. ¹H NMR (500 MHz, DMSO) δ 12.11 (s, 1H), 11.18 (s, 1H), 8.66 (s, 2H), 8.50 (s, 1H), 8.25 (s, 1H), 8.18 (d, J=9.1 Hz, 1H), 8.08 (s, 1H), 7.35 (s, 1H), 7.22 (s, 1H), 7.08 (s, 1H), 6.73 (s, 1H), 5.32 (dd, J=13.0, 5.3 Hz, 1H), 4.17 (q, J=7.1 Hz, 2H), 4.00 (q, J=7.0 Hz, 2H), 3.02-2.90 (m, 4H), 2.77-2.69 (m, 3H), 2.68-2.61 (m, 3H), 2.59-2.51 (m, 3H), 2.49-2.43 (m, 4H), 2.42-2.35 (m, 2H), 2.31 (d, J=8.7 Hz, 5H), 2.17-2.09 (m, 1H), 2.03 (d, J=13.4 Hz, 6H), 1.85 (d, J=10.9 Hz, 2H), 1.56 (d, J=8.9 Hz, 2H), 1.32 (t, J=7.2 Hz, 3H), 1.25 (t, J=6.9 Hz, 3H), 0.87 (s, 3H). [M+H]⁺=1038.4.

### Example 555: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-7-fluoro-2-oxobenzo[d]oxazol-3(2H)-yl)piperidine-2,6-dione

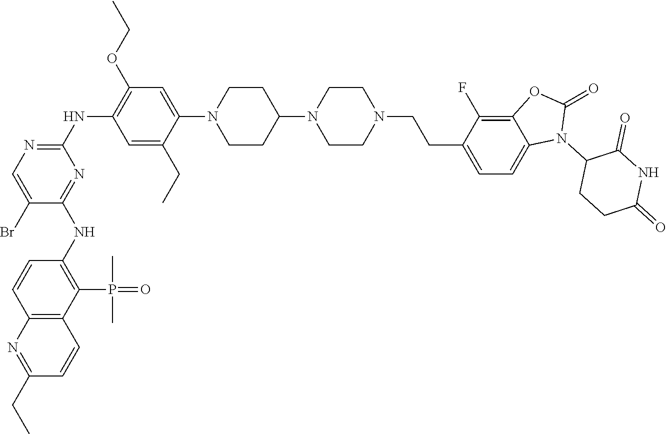 [SMILES: CCOc1cc(N2CCC(N3CCN(CCc4ccc5c(oc(=O)n5C5CCC(=O)NC5=O)c4F)CC3)CC2)c(CC)cc1Nc1ncc(Br)c(Nc2ccc3nc(CC)ccc3c2P(C)(C)=O)n1]

The title compound (29 mg, 41%) was prepared in a manner similar to that in Example 492. ¹H NMR (500 MHz, DMSO) δ 12.11 (s, 1H), 11.18 (s, 1H), 8.66 (s, 1H), 8.50 (s, 1H), 8.25 (s, 1H), 8.18 (d, J=9.1 Hz, 1H), 8.08 (s, 1H), 7.35 (s, 1H), 7.22 (s, 1H), 7.08 (s, 1H), 6.73 (s, 1H), 5.32 (dd, J=13.0, 5.3 Hz, 1H), 4.17 (q, J=7.1 Hz, 2H), 4.00 (q, J=7.0 Hz, 2H), 2.98-2.86 (m, 4H), 2.77-2.69 (m, 3H), 2.68-2.61 (m, 4H), 2.59-2.54 (m, 3H), 2.49-2.43 (m, 4H), 2.42-2.35 (m, 2H), 2.34-2.31 (m, 5H), 2.17-2.09 (m, 1H), 2.03 (d, J=13.4 Hz, 6H), 1.85 (d, J=10.9 Hz, 2H), 1.56 (d, J=8.9 Hz, 2H), 1.32 (t, J=7.2 Hz, 3H), 1.25 (t, J=6.9 Hz, 3H), 0.87 (s, 3H). [M+H]⁺=1038.4.

### Example 556: 3-(7-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)piperidin-1-yl)-5-fluoro-2-oxobenzo[d]oxazol-3(2H)-yl)piperidine-2,6-dione

### Step 1: 2-amino-6-bromo-4-fluorophenol

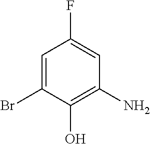

To a solution of 2-bromo-4-fluoro-6-nitrophenol (5 g, 21.3 mmol) in CH₃OH (50 mL) was added Raney-Ni (3 g). The resulting mixture was stirred at rt under hydrogen atmosphere for 3 hrs. The mixture was filtered and concentrated in vacuum to afford 2-amino-6-bromo-4-fluorophenol (3.8 g, 86.6%). [M+H]⁺=206.1.

### Step 2: 7-bromo-5-fluorobenzo[d]oxazol-2(3H)-one

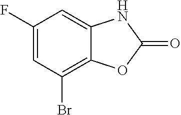 [SMILES: O=c1[nH]c2cc(F)cc(Br)c2o1]

The title compound (4.1 g, 93%) was prepared in a manner similar to that in Example 492 step 1 from 2-amino-6-bromo-4-fluorophenol and CDI. [M+H]⁺=231.9.

### Step 3: 3-2, 6-bis benzyloxy)pyridin-3-yl)-7-bromo-5-fluorobenzo[d]oxazol-2(3H)-one

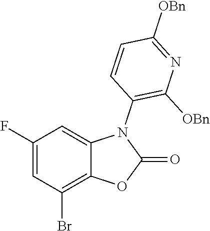 [SMILES: O=c1oc2c(Br)cc(F)cc2n1-c1ccc(OCc2ccccc2)nc1OCc1ccccc1]

The title compound (4.2 g, 93%) was prepared in a manner similar to that in Example 492 step 2 from 7-bromo-5-fluorobenzo[d]oxazol-2(3H)-one and 2,6-bis(benzyloxy)-3-(4,4,5,5-tetramethyl-1,3,2-dioxaborolan-2-yl)pyridine. [M+H]⁺=521.1.

### Step 4: 3-(2,6-bis(benzyloxy)pyridin-3-yl)-5-fluoro-7-(4-(2-hydroxyethyl)piperidin-1-yl)benzo[d]oxazol-2(3H)-one

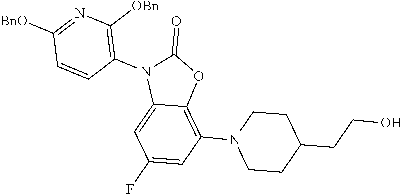 [SMILES: O=c1oc2c(N3CCC(CCO)CC3)cc(F)cc2n1-c1ccc(OCc2ccccc2)nc1OCc1ccccc1]

A mixture of 3-(2,6-bis(benzyloxy)pyridin-3-yl)-7-bromo-5-fluorobenzo[d]oxazol-2(3H)-one (1 g, 1.92 mmol), 2-(piperidin-4-yl)ethan-1-ol (371.5 mg, 2.88 mmol), CuI (73 mg, 0.384 mmol), L-proline (44.1 mg, 0.384 mmol), and K₃PO₄ (814 mg, 3.84 mmol) in DMSO (15 mL) was heated to 100° C. for 18 hrs under nitrogen atmosphere. After cooling to r.t, the reaction was diluted with EA (60 mL) and then washed with brine (20 mL×3), dried over Na₂SO₄, filtered and concentrated in vacuum. The residue was purified by silica gel column (PE:EA=1:1) to afford the product (450 mg, 41.1%). [M+H]⁺=570.3.

### Step 5: 3-(5-fluoro-7-(4-(2-hydroxyethyl)piperidin-1-yl)-2-oxobenzo[d]oxazol-3(2H)-yl)piperidine-2,6-dione

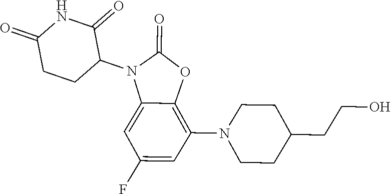 [SMILES: O=C1CCC(n2c(=O)oc3c(N4CCC(CCO)CC4)cc(F)cc32)C(=O)N1]

The title compound (220 mg, 95%) was prepared in a manner similar to that in Example 492 step 4 from 3-(2,6-bis(benzyloxy)pyridin-3-yl)-5-fluoro-7-(4-(2-hydroxyethyl)piperidin-1-yl)benzo[d]oxazol-2(3H)-one. [M+H]⁺=392.3.

### Step 6: 2-(1-(3-(2,6-dioxopiperidin-3-yl)-5-fluoro-2-oxo-2,3-dihydrobenzo[d]oxazol-7-yl)piperidin-4-yl)acetaldehyde

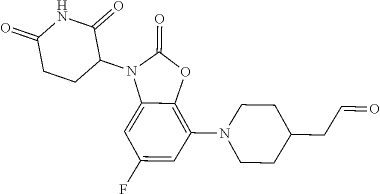 [SMILES: O=CCC1CCN(c2cc(F)cc3c2oc(=O)n3C2CCC(=O)NC2=O)CC1]

The title compound (100 mg, 80%) was prepared in a manner similar to that in Example 492 step 5 from 3-(5-fluoro-7-(4-(2-hydroxyethyl)piperidin-1-yl)-2-oxobenzo[d]oxazol-3(2H)-yl)piperidine-2,6-dione. [M+H]⁺=390.1.

### Step 7: 3-(7-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)piperidin-1-yl)-5-fluoro-2-oxobenzo[d]oxazol-3(2H)-yl)piperidine-2,6-dione

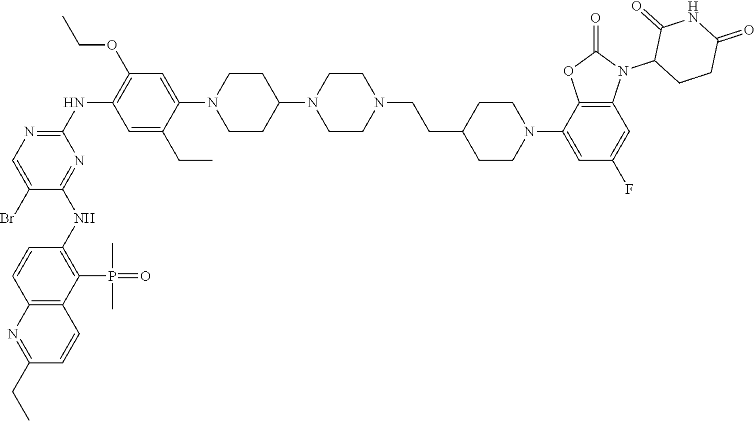 [SMILES: CCOc1cc(N2CCC(N3CCN(CCC4CCN(c5cc(F)cc6c5oc(=O)n6C5CCC(=O)NC5=O)CC4)CC3)CC2)c(CC)cc1Nc1ncc(Br)c(Nc2ccc3nc(CC)ccc3c2P(C)(C)=O)n1]

The title compound (21.45 mg, 43%) was prepared in a manner similar to that in Example 492 step 6 from (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide and 2-(1-(3-(2,6-dioxopiperidin-3-yl)-5-fluoro-2-oxo-2,3-dihydrobenzo [d]oxazol-7-yl)piperidin-4-yl)acetaldehyde. ¹H NMR (500 MHz, DMSO) δ 11.74 (s, 1H), 11.20 (s, 1H), 8.72 (d, J=8.8 Hz, 1H), 8.28 (s, 2H), 8.13 (s, 1H), 7.93 (d, J=9.2 Hz, 1H), 7.51 (d, J=8.9 Hz, 1H), 7.42 (s, 1H), 6.83-6.67 (m, 2H), 6.56 (dd, J=12.9, 2.3 Hz, 1H), 5.33-5.30 (m, 1H), 4.03-4.01 (m, 4H), 3.75-3.73 (m, 3H), 2.99-2.94 (m, 7H), 2.89-2.76 (m, 4H), 2.74-2.60 (m, 5H), 2.54 (s, 1H), 2.24-2.21 (m, 2H), 2.18-2.09 (m, 1H), 2.01 (s, 5H), 1.98 (s, 3H), 1.81-1.78 (m, 2H), 1.75-1.45 (m, 6H), 1.35-1.31 (m, 5H), 1.29-1.24 (m, 4H), 0.70 (s, 3H). [M+H]⁺=1107.9.

### Example 557: 3-(6-(2-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)ethyl)-2-oxobenzo[d]oxazol-3(2H)-yl)piperidine-2,6-dione

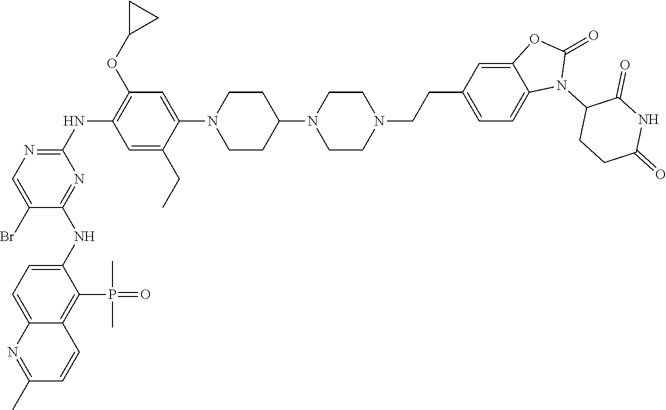 [SMILES: CCc1cc(Nc2ncc(Br)c(Nc3ccc4nc(C)ccc4c3P(C)(C)=O)n2)c(OC2CC2)cc1N1CCC(N2CCN(CCc3ccc4c(c3)oc(=O)n4C3CCC(=O)NC3=O)CC2)CC1]

The title compound (12 mg, 18%) was prepared in a manner similar to that in Example 492. ¹H NMR (500 MHz, DMSO) δ 11.87 (s, 1H), 11.22 (s, 1H), 8.71 (d, J=8.9, 1H), 8.32 (s, 1H), 8.26 (s, 1H), 8.11 (s, 1H), 7.91 (d, J=8.5, 1H), 7.50 (d, J=8.6 Hz, 1H), 7.40 (s, 1H), 7.38 (s, 1H), 7.23 (d, J=8.0 Hz, 1H), 7.14 (d, J=8.0 Hz, 1H), 6.99 (s, 1H), 5.37 (dd, J=12.9, 5.2 Hz, 1H), 3.86-3.80 (m, 1H), 3.75-3.26 (m, 5H), 3.25-2.85 (m, 6H), 2.99-2.83 (m, 4H), 2.76-2.60 (m, 8H), 2.40-2.25 (m, 2H), 2.19-2.13 (m, 1H), 2.15-2.00 (m, 2H), 2.00 (d, J=13.3 Hz, 6H), 1.82-1.67 (m, 2H), 0.86-0.68 (m, 5H), 0.63-0.57 (m, 2H). [M+H]⁺=1005.7.

### Example 558: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3-ethyl-5-fluoro-2-oxo-2,3-dihydro-1H-benzo[d]imidazol-1-yl)piperidine-2,6-dione

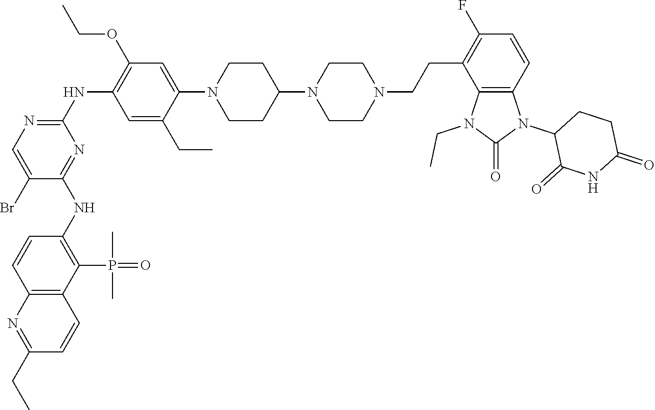 [SMILES: CCOc1cc(N2CCC(N3CCN(CCc4c(F)ccc5c4n(CC)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound (10.53 mg, 10%) was prepared in a manner similar to that in Example 318. ¹H NMR (500 MHz, DMSO) δ 12.11 (s, 1H), 11.11 (s, 1H), 8.73 (d, J=5.9 Hz, 1H), 8.29 (s, 1H), 7.97 (s, 1H), 7.52 (d, J=8.9 Hz, 1H), 7.43 (s, 1H), 7.10 (d, J=9.2 Hz, 2H), 6.96 (d, J=8.6 Hz, 2H), 6.73 (s, 1H), 5.39 (dd, J=12.5, 4.9 Hz, 1H), 4.12-3.91 (m, 4H), 3.73-3.47 (m, 2H), 3.32 (m, 4H), 3.19-2.95 (m, 7H), 2.94-2.83 (m, 1H), 2.72-2.68 (m, 6H), 2.55 (d, J=8.9 Hz, 3H), 2.33-2.21 (m, 2H), 2.22-2.18 (m, 1H), 2.03 (t, J=10.8 Hz, 7H), 1.92-1.77 (m, 2H), 1.32 (t, J=7.2 Hz, 3H), 1.30-1.20 (m, 6H), 0.70 (t, J=6.7 Hz, 3H). [M+H]⁺=1052.3.

### Example 559: 3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-3-ethyl-5-fluoro-2-oxo-2,3-dihydro-1H-benzo[d]imidazol-1-yl)piperidine-2,6-dione

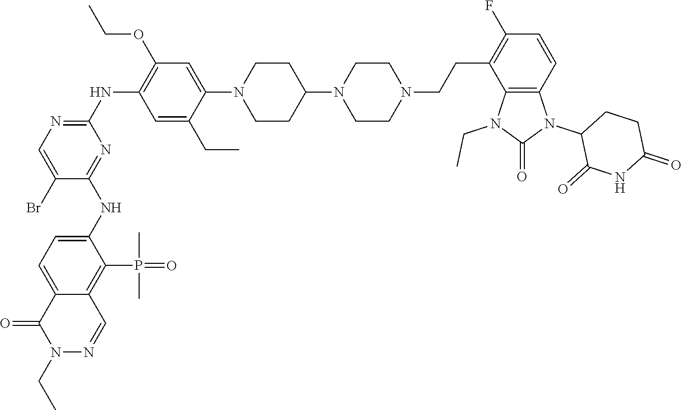 [SMILES: CCOc1cc(N2CCC(N3CCN(CCc4c(F)ccc5c4n(CC)c(=O)n5C4CCC(=O)NC4=O)CC3)CC2)c(CC)cc1Nc1ncc(Br)c(Nc2ccc3c(=O)n(CC)ncc3c2P(C)(C)=O)n1]

The title compound (6.73 mg, 6%) was prepared in a manner similar to that in Example 492. ¹H NMR (500 MHz, DMSO) δ 12.11 (s, 1H), 11.11 (s, 1H), 8.66 (s, 1H), 8.53 (d, J=5.9 Hz, 1H), 8.28 (s, 1H), 8.19 (d, J=8.9 Hz, 1H), 8.12 (s, 1H), 7.37 (s, 1H), 7.04 (s, 1H), 6.93 (t, J=9.6 Hz, 1H), 6.75 (s, 1H), 5.39 (dd, J=12.5, 4.9 Hz, 1H), 4.18 (q, J=7.0 Hz, 2H), 4.12-3.91 (m, 4H), 3.73-3.47 (m, 2H), 3.32 (s, 3H), 3.19-2.95 (m, 7H), 2.94-2.83 (m, 1H), 2.71-2.67 (m, 6H), 2.55 (d, J=10.0 Hz, 2H), 2.46-2.32 (m, 2H), 2.19 (dd, J=6.9, 0.8 Hz, 1H), 2.03 (t, J=10.8 Hz, 7H), 1.92-1.77 (m, 2H), 1.32 (t, J=7.2 Hz, 3H), 1.30-1.20 (m, 6H), 0.89 (t, J=6.5 Hz, 3H). [M+H]⁺=1070.9.

### Example 560: 3-(7-((R)-3-((4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)methyl)pyrrolidin-1-yl)-2-oxobenzo[d]oxazol-3(2H)-yl)piperidine-2,6-dione

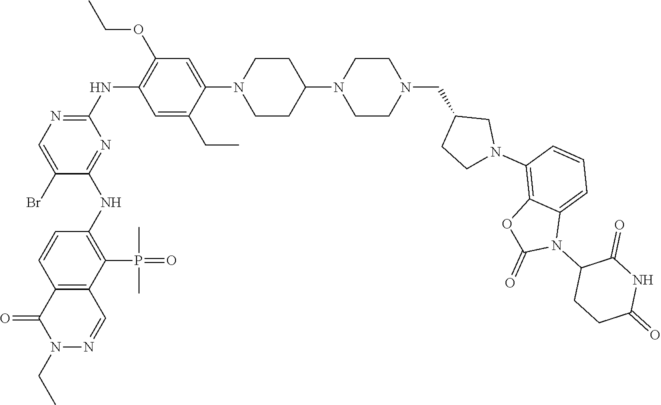 [SMILES: CCOc1cc(N2CCC(N3CCN(C[C@H]4CCN(c5cccc6c5oc(=O)n6C5CCC(=O)NC5=O)C4)CC3)CC2)c(CC)cc1Nc1ncc(Br)c(Nc2ccc3c(=O)n(CC)ncc3c2P(C)(C)=O)n1]

The title compound (32 mg, 45%) was prepared in a manner similar to that in Example 492. ¹H NMR (500 MHz, DMSO) δ 12.12 (s, 1H), 11.18 (s, 1H), 8.61-8.58 (m, 2H), 8.20 (dd, J=21.8, 12.2 Hz, 3H), 7.36 (s, 1H), 7.00 (t, J=8.1 Hz, 1H), 6.73 (s, 1H), 6.50 (d, J=7.8 Hz, 1H), 6.36 (d, J=8.4 Hz, 1H), 5.29 (dd, J=12.9, 5.4 Hz, 1H), 4.17 (q, J=7.0 Hz, 2H), 4.01 (q, J=6.9 Hz, 2H), 3.65-3.41 (m, 5H), 3.27-3.17 (m, 3H), 2.99-2.80 (m, 3H), 2.71-2.53 (m, 8H), 2.36-2.32 (m, 6H), 2.16-1.97 (m, 8H), 1.84 (d, J=10.8 Hz, 2H), 1.67 (dd, J=12.1, 7.7 Hz, 1H), 1.60-1.46 (m, 2H), 1.31 (t, J=7.1 Hz, 3H), 1.25 (t, J=6.9 Hz, 3H), 0.87 (s, 3H). [M+H]⁺=1079.4.

### Example 561: 3-(7-((R)-3-((4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)methyl)pyrrolidin-1-yl)-2-oxobenzo[d]oxazol-3(2H)-yl)piperidine-2,6-dione

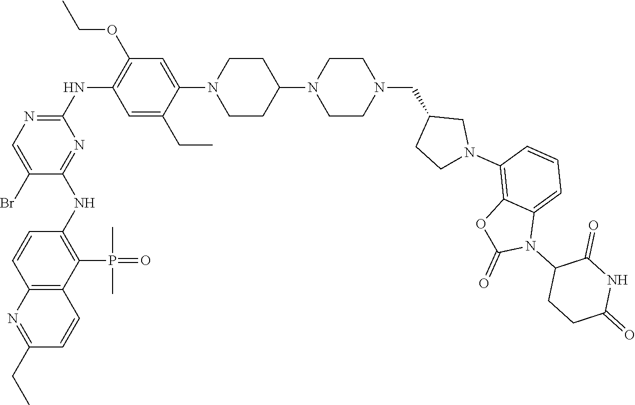 [SMILES: CCOc1cc(N2CCC(N3CCN(C[C@H]4CCN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound (20 mg, 31%) was prepared in a manner similar to that in Example 492. ¹H NMR (500 MHz, DMSO) δ 11.66 (s, 1H), 11.12 (s, 1H), 8.53 (d, J=8.8 Hz, 1H), 8.17 (d, J=13.9 Hz, 2H), 7.89-7.70 (m, 2H), 7.38 (d, J=8.9 Hz, 1H), 7.32 (s, 1H), 6.93 (t, J=8.1 Hz, 1H), 6.64 (s, 1H), 6.43 (d, J=7.8 Hz, 1H), 6.29 (d, J=8.4 Hz, 1H), 5.22 (dd, J=12.9, 5.3 Hz, 1H), 3.93 (q, J=6.9 Hz, 2H), 3.58-3.34 (m, 5H), 3.17-3.06 (m, 2H), 2.95-2.73 (m, 6H), 2.61-2.55 (m, 5H), 2.35-2.11 (m, 8H), 2.10-1.86 (m, 8H), 1.76 (d, J=10.8 Hz, 2H), 1.60 (dd, J=11.8, 7.7 Hz, 1H), 1.52-1.38 (m, 2H), 1.22 (dt, J=13.8, 7.2 Hz, 7H), 0.64 (s, 3H). [M+H]⁺=1064.2.

### Example 562: 3-(7-(3-((4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)methyl)azetidin-1-yl)-2-oxobenzo[d]oxazol-3(2H)-yl)piperidine-2,6-dione

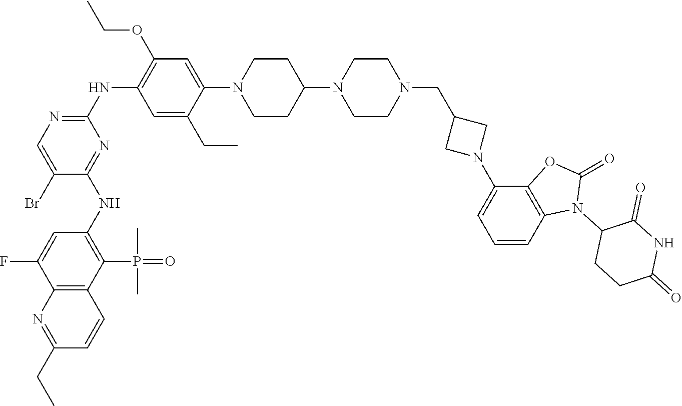 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2cc(F)c3nc(CC)ccc3c2P(C)(C)=O)n1]

The title compound was prepared according to the same procedure as Example 492. ¹H NMR (500 MHz, DMSO) δ 12.10 (s, 1H), 11.19 (s, 1H), 8.56 (d, J=8.8 Hz, 1H), 8.14-8.38 (m, 2H), 8.04 (s, 1H), 7.54 (d, J=9.0 Hz, 1H), 7.39 (s, 1H), 7.01 (t, J=8.0 Hz, 1H), 6.71 (s, 1H), 6.57 (d, J=7.9 Hz, 1H), 6.25 (d, J=8.2 Hz, 1H), 5.29 (dd, J=12.9, 5.3 Hz, 1H), 4.11 (t, J=7.4 Hz, 2H), 4.01 (q, J=6.9 Hz, 2H), 3.65 (t, J=6.5 Hz, 2H), 3.01-2.80 (m, 6H), 2.74-2.52 (m, 10H), 2.46-2.21 (m, 7H), 2.14-2.11 (m, 1H), 2.00 (d, J=13.3 Hz, 6H), 1.84-1.82 (m, 2H), 1.56-1.49 (m, 2H), 1.32 (t, J=7.6 Hz, 3H), 1.26 (t, J=6.9 Hz, 3H), 0.75 (s, 3H). [M+H]⁺=1066.8.

### Example 563: 3-(7-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

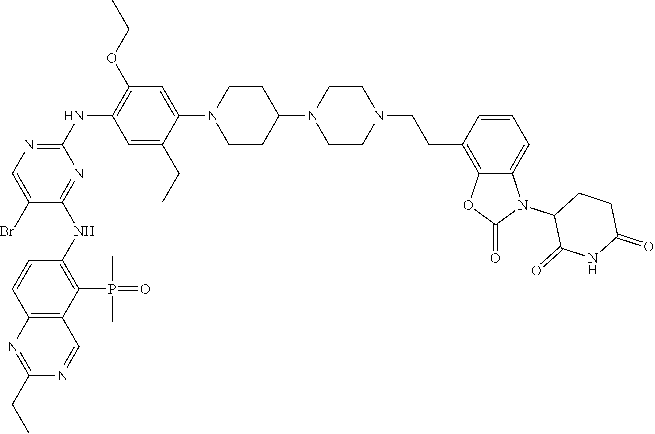 [SMILES: CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3nc(CC)ncc3c2P(C)(C)=O)n1]

The title compound (26 mg, 56%) was prepared in a manner similar to that in Example 492. ¹H NMR (500 MHz, DMSO) δ 11.54 (s, 1H), 11.21 (s, 1H), 9.90 (s, 1H), 8.43 (s, 1H), 8.25 (s, 1H), 7.96 (s, 1H), 7.85 (d, J=9.3 Hz, 1H), 7.32 (s, 1H), 7.15-7.10 (m, 2H), 7.07 (d, J=7.3 Hz, 1H), 6.71 (s, 1H), 5.36 (dd, J=12.7, 5.2 Hz, 1H), 4.00 (t, J=6.9 Hz, 2H), 3.29 (s, 1H), 3.08-3.03 (m, 2H), 2.95-2.82 (m, 5H), 2.67 (s, 1H), 2.63 (s, 3H), 2.61-2.54 (m, 5H), 2.36 (s, 1H), 2.24-2.19 (m, 2H), 2.17-2.13 (m, 1H), 2.03 (d, J=13.4 Hz, 7H), 1.82 (s, 2H), 1.54 (s, 2H), 1.38 (t, J=7.6 Hz, 3H), 1.25 (t, J=6.9 Hz, 6H), 0.70 (s, 3H); [M+H]⁺=1008.7.

### Example 564: 3-(7-(3-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)propyl)-5-fluoro-2-oxobenzo[d]oxazol-3(2H)-yl)piperidine-2,6-dione

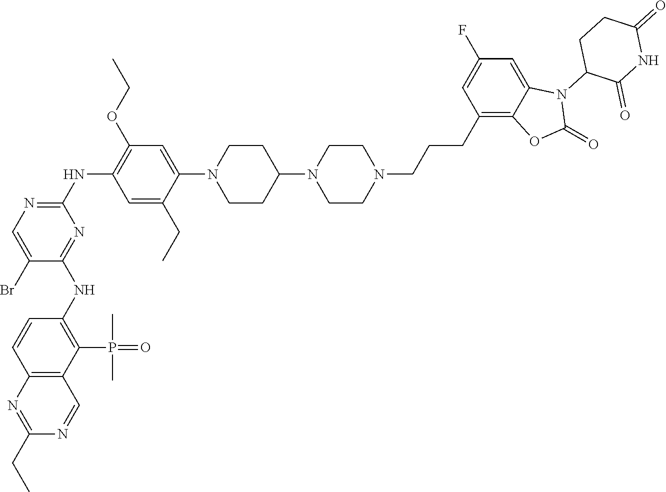 [SMILES: CCOc1cc(N2CCC(N3CCN(CCCc4cc(F)cc5c4oc(=O)n5C4CCC(=O)NC4=O)CC3)CC2)c(CC)cc1Nc1ncc(Br)c(Nc2ccc3nc(CC)ncc3c2P(C)(C)=O)n1]

The title compound (26 mg, 56%) was prepared in a manner similar to that in Example 492. ¹H NMR (500 MHz, DMSO) δ 11.53 (s, 1H), 11.21 (s, 1H), 9.90 (s, 1H), 8.42 (s, 1H), 8.25 (s, 1H), 7.96 (s, 1H), 7.85 (d, J=9.2 Hz, 1H), 7.31 (s, 1H), 7.16 (dd, J=8.4, 2.5 Hz, 1H), 6.92 (dd, J=10.7, 2.4 Hz, 1H), 6.70 (s, 1H), 5.35 (dd, J=13.0, 5.2 Hz, 1H), 3.99 (q, J=7.0 Hz, 2H), 3.30 (s, 2H), 3.05 (q, J=7.5 Hz, 2H), 2.91 (d, J=10.9 Hz, 2H), 2.87-2.82 (m, 1H), 2.75-2.70 (m, 2H), 2.69 (d, J=5.0 Hz, 1H), 2.67 (s, 1H), 2.64-2.62 (m, 2H), 2.60 (s, 1H), 2.54 (s, 2H), 2.37-2.35 (m, 1H), 2.31 (s, 2H), 2.20 (s, 2H), 2.17-2.13 (m, 1H), 2.03 (d, J=13.4 Hz, 7H), 1.83-1.77 (m, 4H), 1.53 (d, J=9.4 Hz, 2H), 1.38 (t, J=7.6 Hz, 3H), 1.25 (t, J=6.9 Hz, 5H), 0.69 (s, 3H); [M+H]⁺=1040.7.

### Example 565: 3-(6-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-5-fluoro-2-oxobenzo[d]oxazol-3(2H)-yl)piperidine-2,6-dione

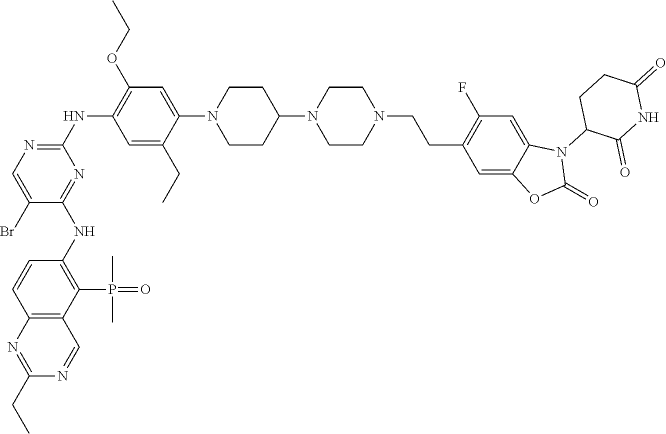 [SMILES: CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2ccc3nc(CC)ncc3c2P(C)(C)=O)n1]

The title compound (26 mg, 56%) was prepared in a manner similar to that in Example 492. ¹H NMR (500 MHz, DMSO) δ 11.54 (s, 1H), 11.20 (s, 1H), 9.89 (s, 1H), 8.43 (s, 1H), 8.25 (s, 1H), 7.96 (s, 1H), 7.85 (d, J=9.1 Hz, 1H), 7.41 (d, J=6.0 Hz, 1H), 7.31 (s, 1H), 7.28 (d, J=9.6 Hz, 1H), 6.70 (s, 1H), 5.34 (dd, J=13.0, 5.2 Hz, 1H), 4.03-3.97 (m, 2H), 3.29 (s, 1H), 3.05 (q, J=7.6 Hz, 2H), 2.91 (d, J=10.3 Hz, 2H), 2.87-2.83 (m, 1H), 2.76 (d, J=7.5 Hz, 2H), 2.70 (d, J=12.6 Hz, 1H), 2.66-2.61 (m, 5H), 2.54 (s, 2H), 2.48-2.43 (m, 3H), 2.36 (s, 1H), 2.28-2.19 (m, 3H), 2.15 (d, J=5.7 Hz, 1H), 2.03 (d, J=13.4 Hz, 7H), 1.82 (d, J=10.2 Hz, 2H), 1.52 (d, J=8.8 Hz, 2H), 1.38 (t, J=7.6 Hz, 3H), 1.25 (t, J=7.0 Hz, 3H), 0.69 (s, 3H); [M+H]⁺=1026.7.

### Example 566: 3-(7-(3-((4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)methyl)azetidin-1-yl)-2-oxobenzo[d]oxazol-3(2H)-yl)piperidine-2,6-dione

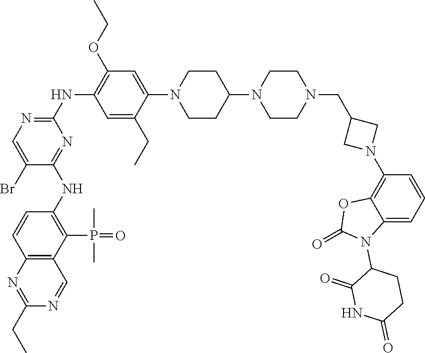 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3nc(CC)ncc3c2P(C)(C)=O)n1]

The title compound (26 mg, 56%) was prepared in a manner similar to that in Example 492. ¹H NMR (500 MHz, DMSO) δ 11.53 (s, 1H), 11.18 (s, 1H), 9.90 (s, 1H), 8.43 (s, 1H), 8.25 (s, 1H), 7.96 (s, 1H), 7.86 (d, J=9.2 Hz, 1H), 7.32 (s, 1H), 7.01 (t, J=8.1 Hz, 1H), 6.71 (s, 1H), 6.57 (d, J=7.8 Hz, 1H), 6.25 (d, J=8.1 Hz, 1H), 5.29 (dd, J=12.9, 5.2 Hz, 1H), 4.11 (t, J=7.4 Hz, 2H), 4.01-3.97 (m, 2H), 3.66 (s, 2H), 3.29 (s, 2H), 3.08-3.04 (m, 2H), 2.91 (d, J=10.8 Hz, 3H), 2.89-2.83 (m, 1H), 2.68-2.57 (m, 7H), 2.54 (s, 1H), 2.40 (s, 3H), 2.36 (s, 1H), 2.20 (s, 2H), 2.14-2.10 (m, 1H), 2.03 (d, J=13.4 Hz, 7H), 1.81 (s, 2H), 1.54 (s, 2H), 1.38 (t, J=7.6 Hz, 3H), 1.25 (t, J=6.9 Hz, 3H), 0.69 (s, 3H); [M+H]⁺=1049.7.

### Example 567: (R)-3-(4-(3-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)propyl)-2,6-difluorophenyl)piperidine-2,6-dione

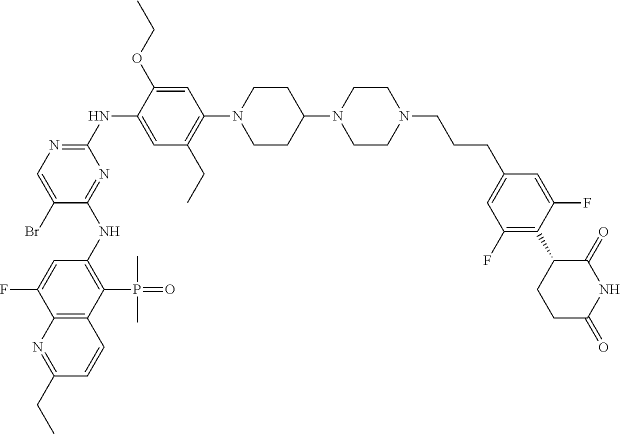 [SMILES: CCOc1cc(N2CCC(N3CCN(CCCc4cc(F)c([C@H]5CCC(=O)NC5=O)c(F)c4)CC3)CC2)c(CC)cc1Nc1ncc(Br)c(Nc2cc(F)c3nc(CC)ccc3c2P(C)(C)=O)n1]

The title compound (26 mg, 56%) was prepared in a manner similar to that in Example 484. ¹H NMR (500 MHz, DMSO) δ 12.10 (s, 1H), 10.95 (s, 1H), 8.56 (d, J=8.6 Hz, 1H), 8.33 (d, J=10.1 Hz, 1H), 8.24 (s, 1H), 8.04 (s, 1H), 7.54 (d, J=9.0 Hz, 1H), 7.39 (s, 1H), 6.99 (d, J=10.1 Hz, 2H), 6.71 (s, 1H), 4.20 (dd, J=12.6, 4.8 Hz, 1H), 4.01 (q, J=6.9 Hz, 2H), 2.95 (dd, J=15.1, 7.7 Hz, 4H), 2.85-2.76 (m, 1H), 2.65-2.53 (m, 8H), 2.49-2.20 (m, 10H), 2.18-2.07 (m, 1H), 2.00 (d, J=13.3 Hz, 7H), 1.82 (d, J=10.7 Hz, 2H), 1.78-1.68 (m, 2H), 1.52 (d, J=9.9 Hz, 2H), 1.32 (t, J=7.6 Hz, 3H), 1.26 (t, J=6.9 Hz, 3H), 0.74 (s, 3H). [M+H]⁺=1018.6.

### Example 568: 3-(6-(2-(4-(1-(4-((5-bromo-4-((5-(dimethylphosphoryl)-2-ethylquinolin-6-yl)amino)pyrimidin-2-yl)amino)-5-cyclopropoxy-2-methylphenyl)piperidin-4-yl)piperazin-1-yl)ethyl)-2-oxobenzo[d]oxazol-3(2H)-yl)piperidine-2,6-dione

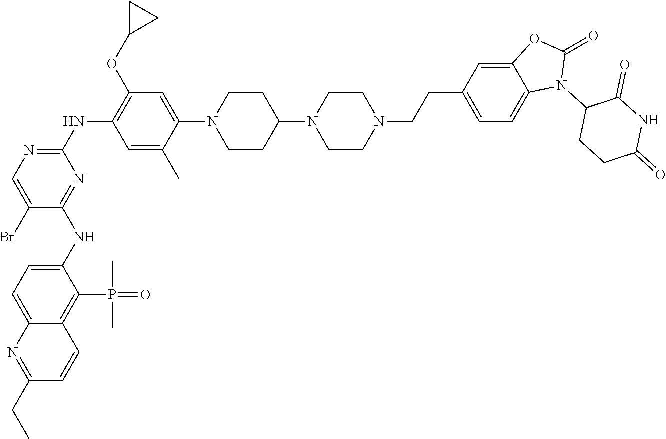 [SMILES: CCc1ccc2c(P(C)(C)=O)c(Nc3nc(Nc4cc(C)c(N5CCC(N6CCN(CCc7ccc8c(c7)oc(=O)n8C7CCC(=O)NC7=O)CC6)CC5)cc4OC4CC4)ncc3Br)ccc2n1]

The title compound (12 mg, 18%) was prepared in a manner similar to that in Example 492 step 6 from (6-((5-bromo-2-((2-cyclopropoxy-5-methyl-4-(4-(piperazin-1-yl)piperidin-1-yl)phenyl)amino)pyrimidin-4-yl)amino)-2-ethylquinolin-5-yl)dimethylphosphine oxide and 2-(3-(2,6-dioxopiperidin-3-yl)-2-oxo-2,3-dihydrobenzo[d]oxazol-6-yl)acetaldehyde. ¹H NMR (500 MHz, DMSO) δ 11.96 (s, 1H), 11.22 (s, 1H), 8.66 (d, J=8.5 Hz, 1H), 8.36 (d, J=8.5 Hz, 1H), 8.25 (s, 1H), 8.08 (s, 1H), 7.93 (d, J=8.6 Hz, 1H), 7.50 (d, J=8.9 Hz, 1H), 7.38 (s, 1H), 7.35 (s, 1H), 7.23 (d, J=7.7 Hz, 1H), 7.14 (d, J=8.1 Hz, 1H), 6.96 (s, 1H), 5.37 (dd, J=12.9, 5.2 Hz, 1H), 3.84 (ddd, J=8.9, 5.8, 2.9 Hz, 1H), 3.60-3.39 (m, 3H), 3.19-3.08 (m, 4H), 3.00-2.85 (m, 7H), 2.75-2.58 (m, 8H), 2.22-2.04 (m, 3H), 2.00 (d, J=13.3 Hz, 6H), 1.90 (s, 3H), 1.80-1.69 (m, 2H), 1.33 (t, J=7.6 Hz, 3H), 0.75-0.69 (m, 2H), 0.64-0.59 (m, 2H).[M+H]⁺=1005.7.

### Example 569: (R)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione

### Step 1: 2,6-bis(benzyloxy)-3-(4-bromophenyl)pyridine

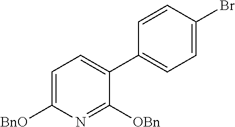 [SMILES: Brc1ccc(-c2ccc(OCc3ccccc3)nc2OCc2ccccc2)cc1]

To a stirred mixture of 2,6-bis(benzyloxy)-3-(4,4,5,5-tetramethyl-1,3,2-dioxaborolan-2-yl)pyridine (The intermediate can be prepared according to the way described in WO2017197046) (8.3 g, 20 mmol) and 4-bromoiodobenzene (5.6 g, 20 mmol) in dioxane (100 mL) and H₂O (10 mL) were added K₂CO₃ (5.5 g, 40 mmol) and Pd(dppf)Cl₂ (1.4 g, 2 mmol) at room temperature under nitrogen atmosphere. The resulting mixture was stirred for 16 h at 80° C. under nitrogen atmosphere. The reaction mixture was allowed to cool down to room temperature. The resulting mixture was extracted with EtOAc (3×500 mL). The combined organic layers were washed with brine (500 mL), dried over anhydrous Na₂SO₄, filtered and concentrated under reduced pressure. The residue was purified by silica gel column chromatography (EA:PE=0-8%) to afford the product (4.5 g, 50%). [M+H]⁺=446.2.

### Step 2: methyl (R)-1-(4-(2,6-bis(benzyloxy)pyridin-3-yl)phenyl)pyrrolidine-3-carboxylate

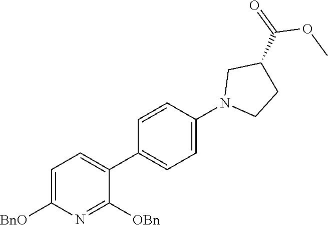 [SMILES: COC(=O)[C@@H]1CCN(c2ccc(-c3ccc(OCc4ccccc4)nc3OCc3ccccc3)cc2)C1]

To the solution of 2,6-bis(benzyloxy)-3-(4-bromophenyl)pyridine (4.5 g, 10 mmol), methyl (R)-pyrrolidine-3-carboxylate hydrochloride (2.5 g, 15 mmol) and Cs₂CO₃ (15 g, 45 mmol) in 50 mL dioxane, was added Pd₂(dba)₃ (915 mg, 1 mmol) and Xantphos (1.5 g, 2 mmol). The mixture was stirred at 80° C. for 16 hours under nitrogen atmosphere. Once the reaction has completed determined by LCMS, the mixture was concentrated under reduced pressure and purified by silica column chromatography (EA:PE=0-12%) to afford the product (2.1 g, 42.5%). [M+H]⁺=494.9.

### Step 3: (R)-1-(4-(2,6-bis(benzyloxy)pyridin-3-yl)phenyl)pyrrolidine-3-carboxylic acid

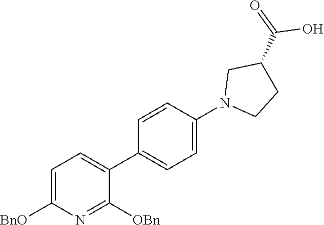 [SMILES: O=C(O)[C@@H]1CCN(c2ccc(-c3ccc(OCc4ccccc4)nc3OCc3ccccc3)cc2)C1]

To the solution of methyl (R)-1-(4-(2,6-bis(benzyloxy)pyridin-3-yl)phenyl)pyrrolidine-3-carboxylate (2.1 g, 4.25 mmol) in 30 mL THF and 10 mL water, was added LiOH H₂O (178 mg, 4.25 mmol) in 2 mL water dropwise at room temperature. The mixture was stirred at room temperature for 15 minutes. Once the reaction has completed determined by TLC, the mixture was concentrated under reduced pressure. The residue was diluted with water and adjust to pH<5 with 1 N HCl aqueous. The liquid was extracted with EtOAc (100 mL×3). The combined organic layers were washed with brine (60 mL×3), dried over anhydrous Na₂SO₄, filtered and concentrated in vacuum to afford the product (2 g, 98%). [M+H]⁺=481.6.

### Step 4: (3R)-1-(4-(2,6-dioxopiperidin-3-yl)phenyl)pyrrolidine-3-carboxylic acid

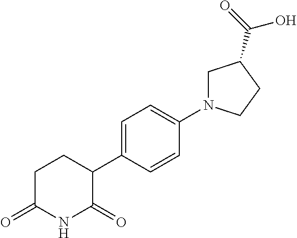 [SMILES: O=C1CCC(c2ccc(N3CC[C@@H](C(=O)O)C3)cc2)C(=O)N1]

To the solution of (R)-1-(4-(2,6-bis(benzyloxy)pyridin-3-yl)phenyl)pyrrolidine-3-carboxylic acid (2 g, 4.17 mmol) in 5 mL DCM and 100 mL iPrOH, was added Pd/C (2 g, 10 wt. %, wet). The mixture was stirred at 45° C. for 16 hours under hydrogen atmosphere (balloon). Once the reaction has completed determined by LCMS, the mixture was cooled to room temperature and filtered through celite directly. The solid was dispensed in DCM (5 mL) and MeOH (50 mL), which was sonicated for 5 min. The mixture was then filtered through celite and the combined filtrate was concentrated in vacuum to afford the product (1.2 g, 95% yield). [M+H]⁺=303.6.

### Step 5: (R)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione

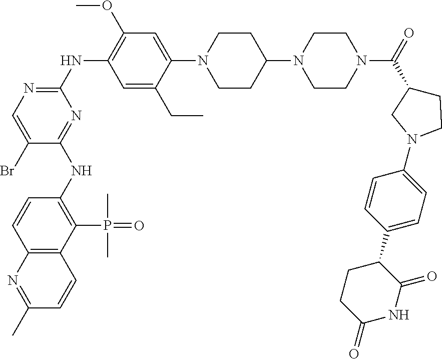 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4ccc([C@H]5CCC(=O)NC5=O)cc4)C3)CC2)CC1]

To the solution of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (140 mg, 0.2 mmol), (3R)-1-(4-(2,6-dioxopiperidin-3-yl)phenyl)pyrrolidine-3-carboxylic acid (60 mg, 0.2 mmol) and DIEA (51 mg, 0.4 mmol) in 5 mL anhydrous DCM, T3P (190 mg, 0.3 mmol, 50% w.t. EtOAc solution) were added. The mixture was stirred at room temperature for 30 minutes. Once the reaction has completed determined by LCMS, the mixture was diluted with 10 mL water. The mixture was extracted with DCM (10 mL×3). The combined organic layers were washed with brine (10 mL×3), dried over anhydrous Na₂SO₄, filtered and concentrated in vacuum. Purification by prep-TLC (DCM:MeOH=15:1) afforded the mixture of two diastereomers, which could be separated by chiral-HPLC (IF (2*25 cm, 5 um), 60% MtBE/40% MeOH:DCM=1:1/0.1% DEA, 80 bar, 20 ml/min) and the title compound corresponded to peak A @ 1.192 min/254 nm) (2.7 mg, 1%). ¹H NMR (500 MHz, DMSO) δ 11.76 (s, 1H), 10.84 (s, 1H), 8.56 (d, J=8.9 Hz, 1H), 8.31 (d, J=5.4 Hz, 1H), 8.21 (s, 1H), 7.98 (s, 1H), 7.87 (d, J=9.2 Hz, 1H), 7.46-7.31 (m, 2H), 7.01 (d, J=9.8 Hz, 2H), 6.74 (s, 1H), 6.23 (d, J=12.2 Hz, 2H), 4.02 (dd, J=12.5, 4.9 Hz, 1H), 3.76 (s, 3H), 3.52-3.48 (m, 6H), 3.38-3.33 (m, 1H), 3.31-3.22 (m, 2H), 2.95 (d, J=10.6 Hz, 2H), 2.83-2.73 (m, 1H), 2.72-2.62 (m, 5H), 2.57 (s, 2H), 2.51 (s, 1H), 2.49-2.45 (m, 2H), 2.37 (dd, J=20.5, 9.9 Hz, 1H), 2.34-2.26 (m, 2H), 2.21-2.04 (m, 3H), 2.02-1.91 (m, 7H), 1.84 (d, J=10.2 Hz, 2H), 1.58 (dd, J=20.3, 10.6 Hz, 2H), 0.87-0.67 (m, 3H). [M+H]⁺=991.3.

### Example 570: 3-(4-(2-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione-3,5,5-d3

### Step 1: (E)-3-(4-(2-ethoxyvinyl)-2,6-difluorophenyl)piperidine-2,6-dione-3,5,5-d3

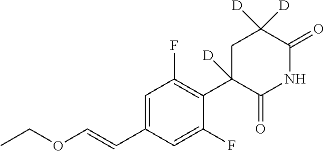 [SMILES: [2H]C1([2H])CC([2H])(c2c(F)cc(/C=C/OCC)cc2F)C(=O)NC1=O]

To a mixture of (E)-3-(4-(2-ethoxyvinyl)-2,6-difluorophenyl)piperidine-2,6-dione (0.4 g, 1.356 mmol, 1 eq) and Et₃N (2.2 g, 21.67 mmol, 16 eq) in 10 mL of ACN was added TMS-Cl (1.18 g, 10.85 mmol, 8 eq) dropwise. The mixture was stirred at 80° C. in a sealed tube for 12 hours. After cooled to 0° C., 5 mL of D2O was added dropwise. The resulting mixture was stirred at room temperature for 2 hours. The mixture was extracted with DCM (3×50 mL) and concentrated to dryness. The residue was purified with silica gel column, eluting with MeOH in DCM (0%-5%) to afford the target compound (0.305 g, 75.5%). ¹H NMR (500 MHz, DMSO) δ 7.98 (s, 1H), 7.00 (d, J=12.9 Hz, 1H), 6.76 (d, J=10.1 Hz, 2H), 5.72 (d, J=12.9 Hz, 1H), 3.91 (q, J=7.0 Hz, 2H), 2.32 (d, J=13.4 Hz, 1H), 2.20-2.08 (m, 1H), 1.35 (t, J=7.0 Hz, 3H); [M+H]⁺=299.1.

### Step 2: 2-(4-(2,6-dioxopiperidin-3-yl-3,5,5-d3)-3,5-difluorophenyl)acetaldehyde

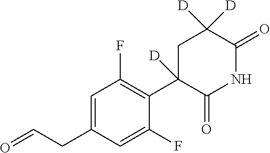 [SMILES: [2H]C1([2H])CC([2H])(c2c(F)cc(CC=O)cc2F)C(=O)NC1=O]

A solution of (E)-3-(4-(2-ethoxyvinyl)-2,6-difluorophenyl)piperidine-2,6-dione-3,5,5-d3 (60 mg, 0.2 mmol) in FA (3 mL) was stirred for 2 hrs at room temperature. The mixture was concentrated in vacuum, and 2-(4-(2,6-dioxopiperidin-3-yl-3,5,5-d3)-3,5-difluorophenyl)acetaldehyde (70 mg, crude) was obtained, which was used in the next step without further purification. [M+H]⁺=271.2.

### Step 3: 3-(4-(2-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione-3,5,5-d3

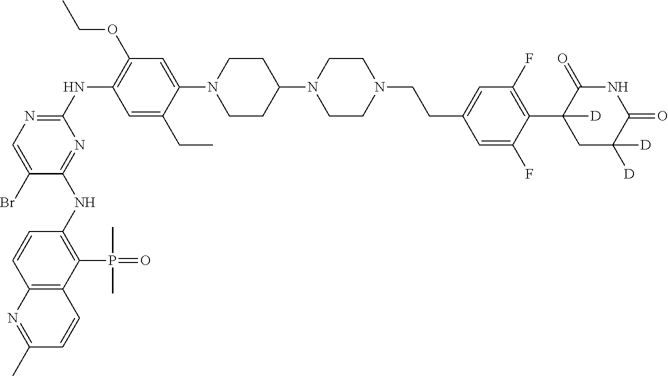

To a solution of 2-(4-(2,6-dioxopiperidin-3-yl-3,5,5-d3)-3,5-difluorophenyl)acetaldehyde (70 mg crude, 0.2 mmol) in DCM (8 mL) was added (6-((5-bromo-2-((2-ethoxy-5-ethyl-4-(4-(piperazin-1-yl)piperidin-1-yl)phenyl)amino)pyrimidin-4-yl)amino)-2-methylquinolin-5-yl)dimethylphosphine oxide (72 mg, 0.1 mmol, the compound was obtained by the way similar to example 314) at room temperature. After 1 h, NaBH(OAc)₃ (42.4 mg, 0.2 mmol) was added to the mixture. The resulting mixture was stirred overnight at room temperature. The mixture was diluted with water (10 mL) and extraction with DCM (3×50 mL). The combined organic phase was washed with brine (20 mL), dried over Na₂SO₄, filtered and concentrated in vacuum. The residue was purified by prep-HPLC (ACN in water with 0.1% of FA, 0% to 90%) to afford the product (7.2 mg, 7.4%). ¹H NMR (500 MHz, DMSO) δ 11.71 (s, 1H), 10.97 (s, 1H), 8.60 (d, J=10.3 Hz, 1H), 8.30-8.24 (m, 1H), 8.22 (s, 1H), 7.91-7.85 (m, 2H), 7.43 (d, J=10.2 Hz, 2H), 7.03 (d, J=10.2 Hz, 2H), 6.07 (s, 1H), 4.00 (dd, J=25, 10.4 Hz, 2H), 2.91 (d, J=10.4 Hz, 2H), 2.75-2.66 (m, 2H), 2.64-2.51 (m, 9H), 2.49-2.08 (m, 9H), 2.00 (s, 3H), 1.96 (s, 3H), 1.83-1.79 (m, 2H), 1.58-1.49 (m, 2H), 1.26 (t, J=10.2 Hz, 3H), 0.71 (t, J=10.1 Hz, 3H); [M+H]⁺=975.2.

### Example 571: 3-(4′-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-2′-oxospiro[cyclopropane-1,3′-indolin]-1′-yl)piperidine-2,6-dione

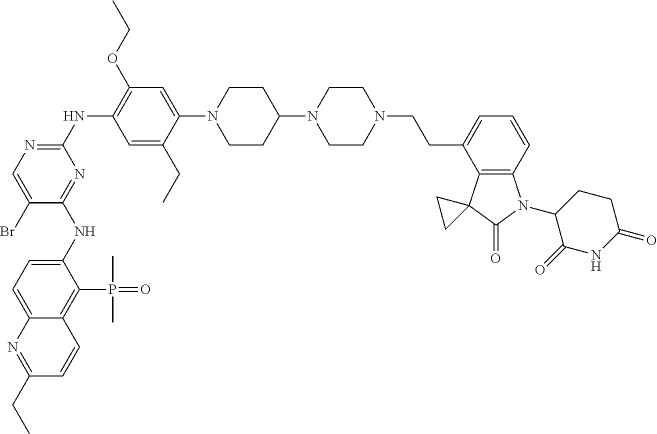 [SMILES: CCOc1cc(N2CCC(N3CCN(CCc4cccc5c4C4(CC4)C(=O)N5C4CCC(=O)NC4=O)CC3)CC2)c(CC)cc1Nc1ncc(Br)c(Nc2ccc3nc(CC)ccc3c2P(C)(C)=O)n1]

The title compound (14 mg, 27%) was prepared in a manner similar to that in Example 492 step 6 from (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide and 2-(1′-(2,6-dioxopiperidin-3-yl)-2′-oxospiro[cyclopropane-1,3′-indolin]-4′-yl)acetaldehyde. ¹H NMR (500 MHz, DMSO) δ 11.67 (s, 1H), 11.09 (s, 1H), 8.64 (s, 1H), 8.24 (s, 2H), 7.90 (s, 2H), 7.46 (d, J=8.9 Hz, 2H), 7.18 (s, 1H), 6.91 (d, J=7.8 Hz, 2H), 6.72 (s, 1H), 5.36-5.31 (m, 1H), 4.02 (q, J=6.9 Hz, 2H), 3.58-3.53 (m, 2H), 3.33-3.29 (m, 4H), 3.20-2.83 (m, 9H), 2.76-2.51 (m, 8H), 2.36 (s, 1H), 2.23 (s, 2H), 2.13 (s, 1H), 1.99-1.95 (m, 9H), 1.75 (s, 1H), 1.56-1.51 (m, 2H), 1.33 (t, J=7.6 Hz, 3H), 1.27 (t, J=6.9 Hz, 3H), 0.69 (s, 3H). [M+H]⁺=1031.7.

### Example 572: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3,3-dimethyl-2-oxoindolin-1-yl)piperidine-2,6-dione

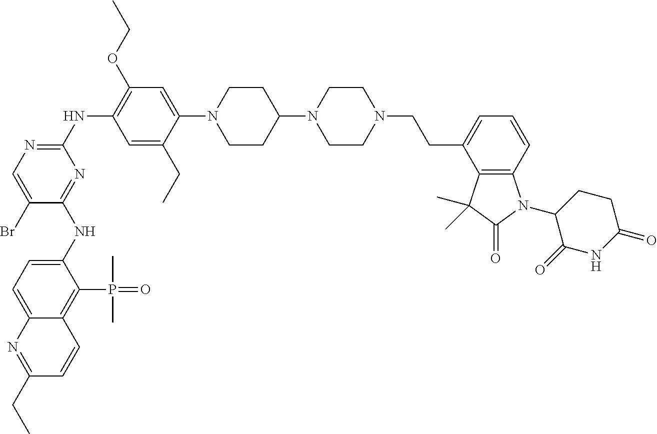 [SMILES: CCOc1cc(N2CCC(N3CCN(CCc4cccc5c4C(C)(C)C(=O)N5C4CCC(=O)NC4=O)CC3)CC2)c(CC)cc1Nc1ncc(Br)c(Nc2ccc3nc(CC)ccc3c2P(C)(C)=O)n1]

The title compound (15 mg, 25%) was prepared in a manner similar to that in Example 492 step 6 from (6-((5-bromo-2-((2-ethoxy-5-ethyl-4-(4-(piperazin-1-yl)piperidin-1-yl)phenyl)amino)pyrimidin-4-yl)amino)-2-ethylquinolin-5-yl)dimethylphosphine oxide and 2-(1-(2,6-dioxopiperidin-3-yl)-3,3-dimethyl-2-oxoindolin-4-yl)acetaldehyde.

¹H NMR (500 MHz, DMSO) δ 11.72 (s, 1H), 11.07 (s, 1H), 8.60 (d, J=9.0 Hz, 1H), 8.25 (s, 1H), 8.22 (s, 1H), 7.92 (s, 1H), 7.88 (d, J=9.2 Hz, 1H), 7.45 (d, J=8.9 Hz, 1H), 7.38 (s, 1H), 7.18 (t, J=7.8 Hz, 1H), 6.91 (d, J=7.8 Hz, 1H), 6.83 (s, 1H), 6.71 (s, 1H), 5.21 (s, 1H), 4.00 (q, J=6.9 Hz, 2H), 3.26-3.16 (m, 2H), 2.90-2.87 (m, 8H), 2.64-2.60 (m, 11H), 2.32-2.18 (m, 3H), 2.01-1.96 (m, 7H), 1.83 (d, J=10.7 Hz, 2H), 1.57-1.51 (m, 2H), 1.38 (s, 6H), 1.32 (t, J=7.6 Hz, 3H), 1.26 (t, J=6.9 Hz, 3H), 0.71 (s, 3H). [M+H]⁺=1033.7.

### Example 573: (R)-3-(4-(3-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)-2-oxoethyl)azetidin-1-yl)-2,6-difluorophenyl)piperidine-2,6-dione

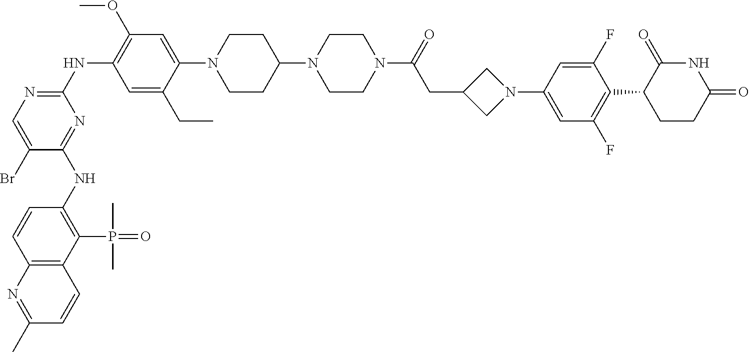 [SMILES: CCc1cc(Nc2ncc(Br)c(Nc3ccc4nc(C)ccc4c3P(C)(C)=O)n2)c(OC)cc1N1CCC(N2CCN(C(=O)CC3CN(c4cc(F)c([C@H]5CCC(=O)NC5=O)c(F)c4)C3)CC2)CC1]

The title compound (10.39 mg, 10%) was prepared in a manner similar to that in Example 488 step 14 from (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide and 2-(1-(4-(2,6-dioxopiperidin-3-yl)-3,5-difluorophenyl)azetidin-3-yl)acetic acid. The product was purified by HPLC (IF (2*25 cm, 5 um), 60% MtBE/40% MeOH:DCM=1:1, 80 bar, 20 ml/min) and the title compound corresponded to peak A @ 1.634 min/254 nm. ¹H NMR (500 MHz, DMSO) δ 11.77 (s, 1H), 10.87 (s, 1H), 8.56 (d, J=8.9 Hz, 1H), 8.30 (dd, J=10.0, 3.9 Hz, 1H), 8.21 (s, 1H), 7.99 (s, 1H), 7.87 (d, J=9.4 Hz, 1H), 7.42 (d, J=8.9 Hz, 1H), 7.37 (s, 1H), 6.74 (s, 1H), 6.10 (d, J=11.2 Hz, 2H), 4.06-3.92 (m, 3H), 3.75 (s, 3H), 3.52-3.39 (m, 6H), 3.02-2.90 (m, 4H), 2.83-2.71 (m, 3H), 2.71-2.61 (m, 5H), 2.49-2.45 (m, 4H), 2.33-2.29 (m, 3H), 2.14-1.90 (m, 8H), 1.82 (d, J=10.8 Hz, 2H), 1.58-1.53 (m, 2H), 0.82-0.71 (m, 3H). [M+H]⁺=1028.4.

### Example 576: (R)-3-(4-((R)-4-(4-(1-(4-((5-bromo-4-((5-(dimethylphosphoryl)-3-fluoro-2-methylquinolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

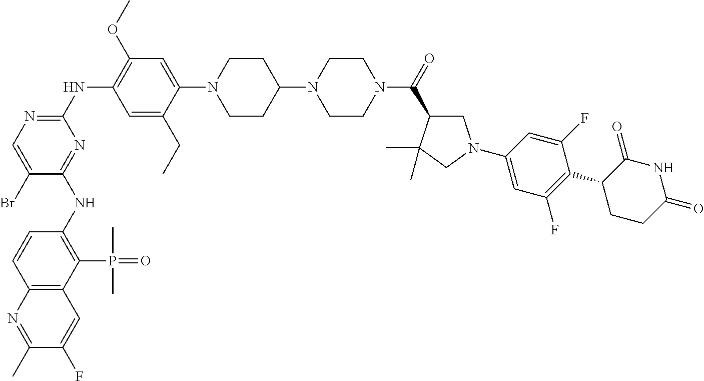 [SMILES: CCc1cc(Nc2ncc(Br)c(Nc3ccc4nc(C)c(F)cc4c3P(C)(C)=O)n2)c(OC)cc1N1CCC(N2CCN(C(=O)[C@H]3CN(c4cc(F)c([C@H]5CCC(=O)NC5=O)c(F)c4)CC3(C)C)CC2)CC1]

The title compound (21 mg, 39%) was prepared in a manner similar to that in Example 488 step 14 from (6-((5-bromo-2-((5-ethyl-2-methoxy-4-(4-(piperazin-1-yl)piperidin-1-yl)phenyl)amino)pyrimidin-4-yl)amino)-3-fluoro-2-methylquinolin-5-yl)dimethylphosphine oxide and (R)-1-(4-((R)-2,6-dioxopiperidin-3-yl)-3,5-difluorophenyl)-4,4-dimethylpyrrolidine-3-carboxylic acid. ¹H NMR (500 MHz, DMSO) δ 11.28 (s, 1H), 10.84 (s, 1H), 8.64 (d, J=12.3 Hz, 1H), 8.22 (s, 1H), 8.18 (s, 1H), 7.95 (d, J=9.2 Hz, 2H), 7.33 (s, 1H), 6.71 (s, 1H), 6.15 (d, J=12.2 Hz, 2H), 4.01 (dd, J=12.4, 5.0 Hz, 1H), 3.75 (s, 3H), 3.66-3.39 (m, TOH), 3.12-3.05 (m, 2H), 2.91 (d, J=11.0 Hz, 2H), 2.83-2.73 (m, 1H), 2.68-2.54 (m, 7H), 2.35 (d, J=11.9 Hz, 1H), 2.19 (s, 2H), 2.07 (t, J=11.0 Hz, 1H), 1.98-1.92 (m, 7H), 1.82 (d, J=11.3 Hz, 2H), 1.55 (d, J=11.1 Hz, 2H), 1.18 (s, 3H), 0.99 (s, 3H), 0.71 (s, 3H). [M+H]⁺=1073.6.

### Example 577: (R)-3-(4-((R)-3-(4-(1-(4-((5-bromo-4-((5-(dimethylphosphoryl)-3-fluoro-2-methylquinolin-6-yl)amino)pyrimidin-2-yl)amino)-5-ethoxy-2-ethylphenyl)piperidin-4-yl)piperazine-1-carbonyl)pyrrolidin-1-yl)-2,6-difluorophenyl)piperidine-2,6-dione

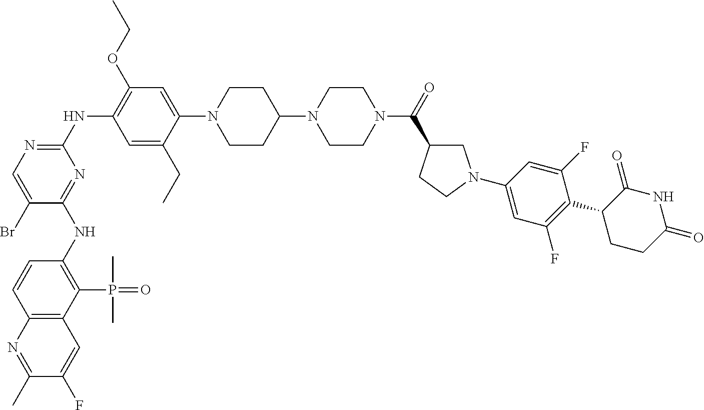 [SMILES: CCOc1cc(N2CCC(N3CCN(C(=O)[C@@H]4CCN(c5cc(F)c([C@H]6CCC(=O)NC6=O)c(F)c5)C4)CC3)CC2)c(CC)cc1Nc1ncc(Br)c(Nc2ccc3nc(C)c(F)cc3c2P(C)(C)=O)n1]

The title compound (27 mg, 55%) was prepared in a manner similar to that in Example 488 step 14 from (6-((5-bromo-2-((2-ethoxy-5-ethyl-4-(4-(piperazin-1-yl)piperidin-1-yl)phenyl)amino)pyrimidin-4-yl)amino)-3-fluoro-2-methylquinolin-5-yl)dimethylphosphine oxide and (R)-1-(4-((R)-2,6-dioxopiperidin-3-yl)-3,5-difluorophenyl)pyrrolidine-3-carboxylic acid. ¹H NMR (500 MHz, DMSO) δ 11.20 (s, 1H), 10.84 (s, 1H), 8.70 (s, 1H), 8.23 (s, 1H), 8.15 (s, 1H), 7.95 (d, J=9.1 Hz, 1H), 7.85 (s, 1H), 7.38 (s, 1H), 6.68 (s, 1H), 6.23 (d, J=12.2 Hz, 2H), 4.00 (d, J=6.9 Hz, 3H), 3.58-3.43 (m, 8H), 3.29-3.23 (m, 3H), 2.89 (d, J=10.4 Hz, 2H), 2.82-2.74 (m, 1H), 2.65-2.63 (m, 8H), 2.34 (d, J=11.2 Hz, 1H), 2.21-2.03 (m, 5H), 1.98-1.91 (m, 7H), 1.81 (d, J=11.2 Hz, 2H), 1.60-1.49 (m, 2H), 1.27 (t, J=6.9 Hz, 3H), 0.64 (s, 3H). [M+H]⁺=1059.6.

### Example 578: (R)-3-(4-(4-(2-(4-(1-(4-((5-bromo-4-((2-cyclopropyl-5-(dimethylphosphoryl)-1-oxo-1,2-dihydrophthalazin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

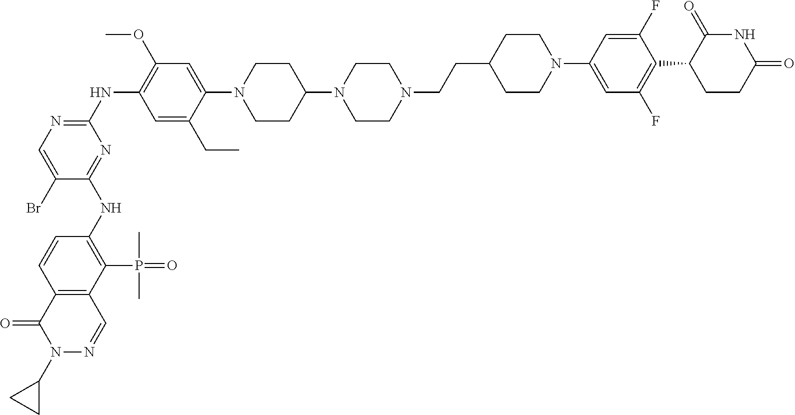 [SMILES: CCc1cc(Nc2ncc(Br)c(Nc3ccc4c(=O)n(C5CC5)ncc4c3P(C)(C)=O)n2)c(OC)cc1N1CCC(N2CCN(CCC3CCN(c4cc(F)c([C@H]5CCC(=O)NC5=O)c(F)c4)CC3)CC2)CC1]

The title compound (19 mg, 20%) was prepared in a manner similar to that in Example 484. ¹H NMR (500 MHz, DMSO) δ 12.23 (s, 1H), 10.86 (s, 1H), 8.58 (s, 1H), 8.54 (s, 1H), 8.25 (s, 1H), 8.23-8.13 (m, 2H), 7.29 (s, 1H), 6.77 (s, 1H), 6.61 (d, J=12.8 Hz, 2H), 4.06-4.03 (m, 2H), 3.74-3.72 (m, 5H), 3.01-2.99 (m, 2H), 2.80-2.61 (m, 6H), 2.48-2.18 (m, 8H), 2.09-2.07 (m, 1H), 2.05-2.02 (m, 7H), 1.95 (s, 1H), 1.84 (s, 2H), 1.73-1.70 (m, 2H), 1.57-1.54 (m, 2H), 1.52-1.42 (m, 2H), 1.39-1.36 (m, 2H), 1.29-1.10 (m, 4H), 1.06-0.88 (m, 8H); [M+H]⁺=1084.5.

### Example 581: 3-(7-(4-((4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)methyl)piperidin-1-yl)-2-oxobenzo[d]oxazol-3(2H)-yl)piperidine-2,6-dione

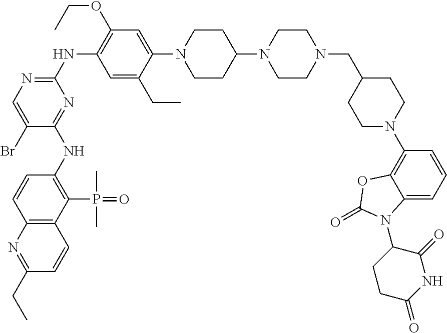 [SMILES: CCOc1cc(N2CCC(N3CCN(CC4CCN(c5cccc6c5oc(=O)n6C5CCC(=O)NC5=O)CC4)CC3)CC2)c(CC)cc1Nc1ncc(Br)c(Nc2ccc3nc(CC)ccc3c2P(C)(C)=O)n1]

The title compound (25.5 mg, 35%) was prepared in a manner similar to that in Example 492. ¹H NMR (500 MHz, DMSO) δ 11.88 (s, 1H), 11.21 (s, 1H), 8.76 (d, J=8.6 Hz, 1H), 8.29 (d, J=11.8 Hz, 3H), 7.95 (d, J=9.2 Hz, 1H), 7.54 (d, J=8.9 Hz, 1H), 7.42 (s, 1H), 7.10 (t, J=8.1 Hz, 1H), 6.79 (d, J=7.9 Hz, 1H), 6.76-6.70 (m, 2H), 5.35-5.31 (m, 1H), 4.03 (d, 2H), 3.70 (d, 3H), 3.57 (s, 3H), 3.47-3.44 (m, 2H), 3.23 (s, 3H), 3.01-2.97 (m, 5H), 2.87-2.84 (m, 5H), 2.75-2.62 (m, 4H), 2.29-2.25 (m, 2H), 2.09 (d, 2H), 2.00 (d, 6H), 1.87 (d, 3H), 1.75 (d, J=9.7 Hz, 2H), 1.38-1.33 (m, 5H), 1.28 (t, 3H), 0.70 (s, 3H). [M+H]⁺=1076.4

### Example 582: 3-(7-(4-((4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)methyl)piperidin-1-yl)-2-oxobenzo[d]oxazol-3(2H)-yl)piperidine-2,6-dione

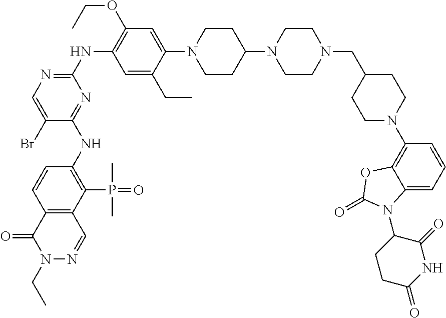 [SMILES: CCOc1cc(N2CCC(N3CCN(CC4CCN(c5cccc6c5oc(=O)n6C5CCC(=O)NC5=O)CC4)CC3)CC2)c(CC)cc1Nc1ncc(Br)c(Nc2ccc3c(=O)n(CC)ncc3c2P(C)(C)=O)n1]

The title compound (27.5 mg, 31.5%) was prepared in a manner similar to that in Example 492. ¹H NMR (500 MHz, DMSO) 12.12 (s, 1H), 11.20 (s, 1H), 8.67 (s, 1H), 8.50 (s, 1H), 8.33-8.15 (m, 2H), 8.06 (s, 1H), 7.36 (s, 1H), 7.08 (t, 1H), 6.83-6.65 (m, 3H), 5.35-5.31 (m, 1H), 4.19-4.15 (m, 2H), 4.05-4.00 (m, 2H), 3.66 (d, 2H), 2.96 (d, 2H), 2.88 (t, 1H), 2.75 (t, 2H), 2.65 (d, 4H), 2.54 (s, 4H), 2.38 (d, 5H), 2.31 (d, 2H), 2.21-2.10 (m, 3H), 2.03 (d, 6H), 1.83 (t, J=13.7 Hz, 4H), 1.69 (s, 1H), 1.55 (d, J=10.5 Hz, 2H), 1.31 (t, J=7.1 Hz, 3H), 1.25 (t, J=6.8 Hz, 4H), 1.06 (t, J=7.0 Hz, 1H), 0.87 (s, 3H). [M+H]⁺=1093.4.

### Example 583: 3-(4-(2-(1′-(4-((5-bromo-4-((5-(dimethylphosphoryl)-2-ethylquinolin-6-yl)amino)pyrimidin-2-yl)amino)-2-ethyl-5-methoxyphenyl)-[1,4′-bipiperidin]-4-yl)ethyl)-2,6-difluorophenyl)piperidine-2,6-dione

### Step 1: ((1-(tert-butoxycarbonyl)piperidin-4-yl)methyl)triphenylphosphonium iodide

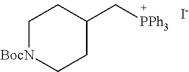

To a solution of tert-butyl 4-(iodomethyl)piperidine-1-carboxylate (1.5 g, 4.62 mmol) in DMF (20 mL) was added triphenylphosphane (1.33 g, 5.08 mmol) at room temperature. The resulting mixture was stirred at 100° C. overnight. The reaction was concentrated and 20 mL EA was added. Then the mixture was filtered to give the desired product (2 g, 71%). [M+H]⁺=460.2.

### Step 2: tert-butyl(E)-4-(4-(2,6-bis(benzyloxy)pyridin-3-yl)-3,5-difluorostyryl)piperidine-1-carboxylate

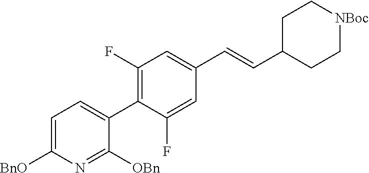 [SMILES: CC(C)(C)OC(=O)N1CCC(/C=C/c2cc(F)c(-c3ccc(OCc4ccccc4)nc3OCc3ccccc3)c(F)c2)CC1]

To a solution of ((1-(tert-butoxycarbonyl)piperidin-4-yl)methyl)triphenylphosphonium iodide (2 g, 3.41 mmol) in THF (10 mL) was added LiHMDS (1.0 M in THF, 3.5 mL, 3.5 mmol) at −40° C. The mixture was stirred at −40° C. for 0.5 h. Then 4-(2,6-bis(benzyloxy)pyridin-3-yl)-3,5-difluorobenzaldehyde (1.5 g, 3.5 mmol) in 5 mL THF was added. The reaction was stirred at −40° C. for 3 h and quenched with sat. NH₄Cl (30 mL). The resulting mixture was extracted with EA (2×100 mL) and the combined organic layers were dried over anhydrous Na₂SO₄, filtered and evaporated in vacuum to afford the crude residue, which was purified by silica gel column chromatography (PE: EA=100: 0-10: 1 gradient elution) to give the desired product (0.9 g, 43%). [M+H]⁺=613.3.

### Step 3: tert-butyl 4-(4-(2,6-dioxopiperidin-3-yl)-3,5-difluorophenethyl)piperidine-1-carboxylate

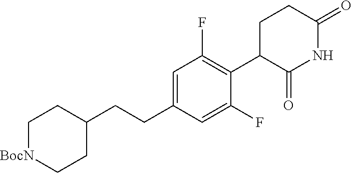 [SMILES: CC(C)(C)OC(=O)N1CCC(CCc2cc(F)c(C3CCC(=O)NC3=O)c(F)c2)CC1]

To a suspension of tert-butyl(E)-4-(4-(2,6-bis(benzyloxy)pyridin-3-yl)-3,5-difluorostyryl)piperidine-1-carboxylate (0.45 g, 0.74 mmol) in MeOH (10 mL) was added Pd/C (0.5 g, 10 wt. %, wet). The mixture was stirred at rt for 16 hrs under hydrogen atmosphere. Then the mixture was filtered and washed with MeOH. The filtrate was concentrated in vacuo to give the desired product (230 mg, 72%). [M+H]⁺=437.2.

### Step 4: 3-(2,6-difluoro-4-(2-(piperidin-4-yl)ethyl)phenyl)piperidine-2,6-dione

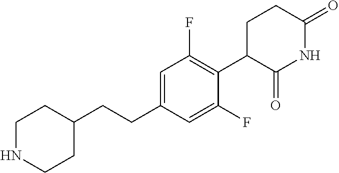 [SMILES: O=C1CCC(c2c(F)cc(CCC3CCNCC3)cc2F)C(=O)N1]

A solution of tert-butyl 4-(4-(2,6-dioxopiperidin-3-yl)-3,5-difluorophenethyl)piperidine-1-carboxylate (230 mg, 0.53 mmol) in TFA (5 mL) was stirred at r.t. for 2 h. The reaction was concentrated to near dryness and basified with sat. aq. NaHCO₃, then extracted with DCM (2×100 mL). The combined organic layers were dried over anhydrous Na₂SO₄, filtered and evaporated in vacuum to afford the crude residue, which was purified by silica gel column chromatography (DCM: MeOH=100: 0-20: 1 gradient elution) to give the desired product (160 mg, 90%). [M+H]⁺=337.2.

### Step 5: 3-(4-(2-(1′-(2-ethyl-5-methoxy-4-nitrophenyl)-[1,4′-bipiperidin]-4-yl)ethyl)-2,6-difluorophenyl)piperidine-2,6-dione

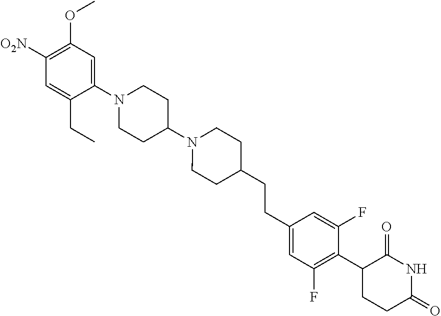 [SMILES: CCc1cc([N+](=O)[O-])c(OC)cc1N1CCC(N2CCC(CCc3cc(F)c(C4CCC(=O)NC4=O)c(F)c3)CC2)CC1]

A solution of 3-(2,6-difluoro-4-(2-(piperidin-4-yl)ethyl)phenyl)piperidine-2,6-dione (160 mg, 0.48 mmol), 1-(2-ethyl-5-methoxy-4-nitrophenyl)piperidin-4-one (132 mg, 0.48 mmol) and titanium(IV) isopropoxide (270 mg, 0.96 mmol) in DCE (10 mL) was stirred at 50° C. overnight. The reaction was cooled to r.t. and STAB (201 mg, 0.96 mmol) was added. The reaction was stirred at r.t. for 2 h and 100 mL EA was added. The mixture was washed with sat. aq. NaCl (2×40 mL). The organic layer was dried over anhydrous Na₂SO₄, filtered and evaporated in vacuum to afford the crude residue, which was purified by silica gel column chromatography (PE: EA=100: 0-50: 1 gradient elution) to give the desired product (130 mg, 46%). [M+H]⁺=599.3.

### Step 6: 3-(4-(2-(1′-(4-amino-2-ethyl-5-methoxyphenyl)-[1,4′-bipiperidin]-4-yl)ethyl)-2,6-difluorophenyl)piperidine-2,6-dione

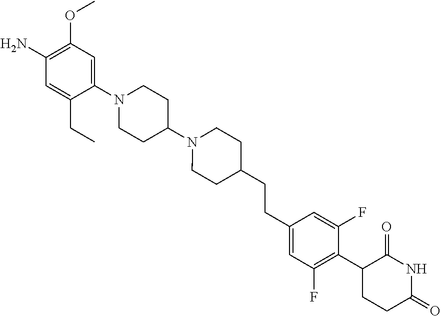 [SMILES: CCc1cc(N)c(OC)cc1N1CCC(N2CCC(CCc3cc(F)c(C4CCC(=O)NC4=O)c(F)c3)CC2)CC1]

A solution of 3-(4-(2-(1′-(2-ethyl-5-methoxy-4-nitrophenyl)-[1,4′-bipiperidin]-4-yl)ethyl)-2,6-difluorophenyl)piperidine-2,6-dione (130 mg, 0.22 mmol) and Zn (139 mg, 2.2 mmol) in AcOH (5 mL) was stirred at 60° C. for 3 h. Then the mixture was adjusted to pH=8 with sat. aq. NaHCO₃ and extracted with DCM (2×100 mL). The organic phase was washed with brine (1×80 mL), dried over Na₂SO₄, filtered and concentrated in vacuum to afford the desired product (90 mg, 73%). [M+H]⁺=569.3.

### Step 7: 3-(4-(2-(1′-(4-((5-bromo-4-((5-(dimethylphosphoryl)-2-ethylquinolin-6-yl)amino)pyrimidin-2-yl)amino)-2-ethyl-5-methoxyphenyl)-[1,4′-bipiperidin]-4-yl)ethyl)-2,6-difluorophenyl)piperidine-2,6-dione

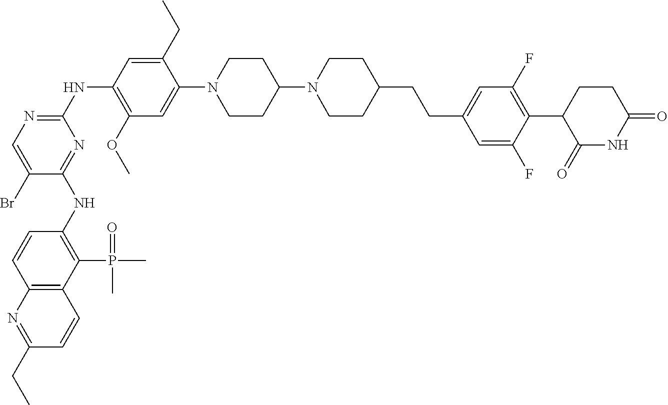 [SMILES: CCc1ccc2c(P(C)(C)=O)c(Nc3nc(Nc4cc(CC)c(N5CCC(N6CCC(CCc7cc(F)c(C8CCC(=O)NC8=O)c(F)c7)CC6)CC5)cc4OC)ncc3Br)ccc2n1]

A solution of 3-(4-(2-(1′-(4-amino-2-ethyl-5-methoxyphenyl)-[1,4′-bipiperidin]-4-yl)ethyl)-2,6-difluorophenyl)piperidine-2,6-dione (40 mg, 0.07 mmol), (6-((5-bromo-2-chloropyrimidin-4-yl)amino)-2-ethylquinolin-5-yl)dimethylphosphine oxide (31 mg, 0.07 mmol) and TsOH (36 mg, 0.21 mmol) in 2-methyl-2-butano (5 mL) was stirred at 100° C. for 4 h. Then the mixture was adjusted to pH=8 with sat. aq. NaHCO₃ and extracted with DCM (2×100 mL). The organic phase was washed with brine (1×80 mL), dried over Na₂SO₄, filtered and evaporated in vacuum to afford the crude residue, which was purified by silica gel column chromatography (DCM: MeOH=100: 0-10: 1 gradient elution) to give an impure product, which was further purified with prep-HPLC to give the desired product (10.38 mg, 19%). ¹H NMR (500 MHz, DMSO) δ 11.80 (s, 1H), 10.94 (s, 1H), 8.55 (d, J=8.9 Hz, 1H), 8.27 (s, 1H), 8.21 (s, 1H), 8.00 (s, 1H), 7.87 (d, J=9.3 Hz, 1H), 7.44 (d, J=8.9 Hz, 1H), 7.32 (s, 1H), 6.98 (d, J=10.0 Hz, 2H), 6.74 (s, 1H), 4.19 (dd, J=12.6, 5.1 Hz, 1H), 3.75 (s, 3H), 3.31-3.28 (m, 1H), 2.92 (dt, J=16.0, 8.0 Hz, 6H), 2.82 (dd, J=21.8, 8.9 Hz, 1H), 2.71-2.59 (m, 4H), 2.40-2.27 (m, 3H), 2.20-2.08 (m, 3H), 1.98 (d, J=13.3 Hz, 7H), 1.80 (d, J=11.9 Hz, 2H), 1.72 (d, J=10.7 Hz, 2H), 1.63-1.47 (m, 4H), 1.32 (t, J=7.6 Hz, 3H), 1.25-1.11 (m, 3H), 0.77 (s, 3H); [M+H]⁺=971.7.

### Example 584: 3-(4-(2-(4-(1-(4-((5-bromo-4-((5-(dimethylphosphoryl)-2-ethylquinolin-6-yl)amino)pyrimidin-2-yl)amino)-5-cyclopropoxy-2-methylphenyl)piperidin-4-yl)piperazin-1-yl)ethyl)-3-methyl-2-oxo-2,3-dihydro-1H-benzo[d]imidazol-1-yl)piperidine-2,6-dione

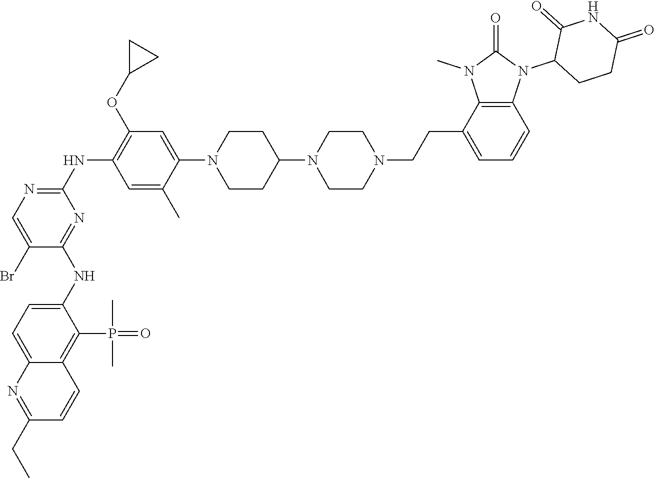 [SMILES: CCc1ccc2c(P(C)(C)=O)c(Nc3nc(Nc4cc(C)c(N5CCC(N6CCN(CCc7cccc8c7n(C)c(=O)n8C7CCC(=O)NC7=O)CC6)CC5)cc4OC4CC4)ncc3Br)ccc2n1]

The title compound (32 mg, 46%) was prepared in a manner similar to that in Example 318. ¹H NMR (500 MHz, DMSO) δ 11.88 (s, 1H), 11.09 (s, 1H), 8.55 (d, J=8.8 Hz, 1H), 8.34 (dd, J=5.1 Hz, 1H), 8.19 (s, 1H), 7.91-7.84 (m, 2H), 7.44 (d, J=8.9 Hz, 1H), 7.30 (s, 1H), 7.01-6.94 (m, 3H), 6.91 (dd, J=6.2, 2.6 Hz, 1H), 5.37 (dd, J=12.8, 5.4 Hz, 1H), 3.81 (tt, J=5.9, 2.9 Hz, 1H), 3.59 (s, 3H), 3.12-3.01 (m, 4H), 2.97-2.84 (m, 3H), 2.78-2.52 (m, 13H), 2.31 (t, J=11.2 Hz, 1H), 2.07-1.91 (m, 8H), 1.94-1.81 (m, 5H), 1.57 (dd, J=20.2, 11.2 Hz, 2H), 1.32 (t, J=7.6 Hz, 3H), 0.70 (q, J=6.0 Hz, 2H), 0.61-0.55 (m, 2H). [M+H]⁺=1018.3

### Example 585: 3-(4-(2-(4-(1-(4-((5-bromo-4-((5-(dimethylphosphoryl)-2-ethylquinolin-6-yl)amino)pyrimidin-2-yl)amino)-5-cyclopropoxy-2-ethylphenyl)piperidin-4-yl)piperazin-1-yl)ethyl)-3-methyl-2-oxo-2,3-dihydro-1H-benzo[d]imidazol-1-yl)piperidine-2,6-dione

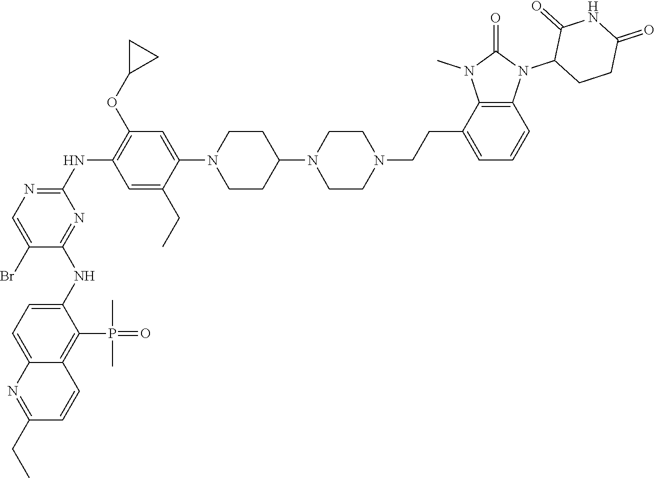 [SMILES: CCc1ccc2c(P(C)(C)=O)c(Nc3nc(Nc4cc(CC)c(N5CCC(N6CCN(CCc7cccc8c7n(C)c(=O)n8C7CCC(=O)NC7=O)CC6)CC5)cc4OC4CC4)ncc3Br)ccc2n1]

The title compound (29 mg, 40%) was prepared in a manner similar to that in Example 318. ¹H NMR (500 MHz, DMSO) δ 11.78 (s, 1H), 11.09 (s, 1H), 8.57 (d, J=8.8 Hz, 1H), 8.25 (s, 1H), 8.20 (s, 1H), 7.91-7.82 (m, 2H), 7.44 (d, J=8.9 Hz, 1H), 7.34 (s, 1H), 7.03-6.95 (m, 3H), 6.90 (dd, J=6.2, 2.6 Hz, 1H), 5.37 (dd, J=12.8, 5.4 Hz, 1H), 3.81 (tt, J=6.0, 3.0 Hz, 1H), 3.59 (s, 3H), 3.10-3.02 (m, 2H), 2.99-2.86 (m, 5H), 2.78-2.51 (m, 13H), 2.30 (t, J=9.1 Hz, 3H), 1.98 (d, J=13.3 Hz, 8H), 1.85 (d, J=10.9 Hz, 2H), 1.55 (dd, J=20.2, 11.4 Hz, 2H), 1.32 (t, J=7.6 Hz, 3H), 0.82-0.67 (m, 5H), 0.61-0.55 (m, 2H). [M+H]⁺=1032.8.

### Example 586: (R)-3-(4-((R)-3-(4-(1-(4-((5-bromo-4-((8-(dimethylphosphoryl)-3-methylisoquinolin-7-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

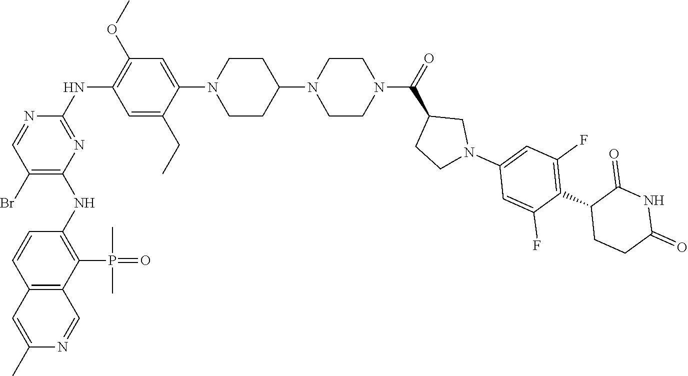 [SMILES: CCc1cc(Nc2ncc(Br)c(Nc3ccc4cc(C)ncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound (10 mg, 30%) was prepared in a manner similar to that in Example 488 step 14 from (7-((5-bromo-2-((5-ethyl-2-methoxy-4-(4-(piperazin-1-yl)piperidin-1-yl)phenyl)amino)pyrimidin-4-yl)amino)-3-methylisoquinolin-8-yl)dimethylphosphine oxide and (R)-1-(4-((R)-2,6-dioxopiperidin-3-yl)-3,5-difluorophenyl)pyrrolidine-3-carboxylic acid. ¹H NMR (500 MHz, DMSO) δ 11.84 (s, 1H), 10.84 (s, 1H), 9.47 (s, 1H), 8.28 (s, 1H), 8.23 (s, 1H), 7.95 (s, 1H), 7.86 (d, J=9.0 Hz, 1H), 7.66 (s, 1H), 7.42 (s, 1H), 6.73 (s, 1H), 6.24 (s, 1H), 6.22 (s, 1H), 4.02 (dd, J=12.7, 4.9 Hz, 1H), 3.76 (s, 3H), 3.56-3.43 (m, 7H), 3.34 (d, J=6.5 Hz, 2H), 3.31-3.22 (m, 4H), 2.94 (d, J=10.8 Hz, 2H), 2.68-2.63 (m, 2H), 2.62 (s, 3H), 2.55 (d, J=14.8 Hz, 2H), 2.30 (d, J=7.0 Hz, 2H), 2.22-2.12 (m, 2H), 2.10 (d, J=6.7 Hz, 2H), 2.05 (d, J=13.3 Hz, 6H), 1.96-1.93 (m, 1H), 1.83 (d, J=11.2 Hz, 2H), 1.64-1.53 (m, 2H), 0.75 (s, 3H). [M+H]⁺=1027.9.

### Example 587: N-(6-((5-bromo-2-((4-(4-(4-((R)-1-(4-((R)-2,6-dioxopiperidin-3-yl)-3,5-difluorophenyl)pyrrolidine-3-carbonyl)piperazin-1-yl)piperidin-1-yl)-5-ethyl-2-methoxyphenyl)amino)pyrimidin-4-yl)amino)-5-(dimethylphosphoryl)quinolin-2-yl)cyclopropanecarboxamide

### Step 1: 2-chloroquinolin-6-amine

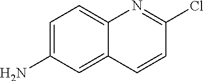 [SMILES: Nc1ccc2nc(Cl)ccc2c1]

A mixture of 2-chloro-6-nitroquinoline (12 g, 57.7 mmol) and Fe (16 g, 0.29 mol) in EtOH (50 mL) and sat. NH₄Cl (200 mL) was stirred at 80° C. for 4 hrs. After cooling to r.t, the reaction mixture was filtered, and the filtrate was extracted with EA (3×200 mL). The combined organic layers were washed with brine (2×20 mL), dried over Na₂SO₄, filtered and concentrated under vacuum to afford the crude residue, which was purified with silica gel column chromatography (PE: EA=2:1) to give the product (9 g, 87%). [M+H]⁺=179.1.

### Step 2: 2-chloro-5-iodoquinolin-6-amine

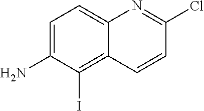 [SMILES: Nc1ccc2nc(Cl)ccc2c1I]

The title compound (9 g, 59%) was prepared in a manner similar to that in Example 486 step 4 from 2-chloroquinolin-6-amine and ICL. [M+H]⁺=305.0.

### Step 3: (6-amino-2-chloroquinolin-5-yl)dimethylphosphine oxide

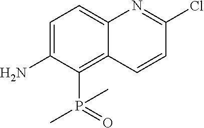 [SMILES: CP(C)(=O)c1c(N)ccc2nc(Cl)ccc12]

The title compound (6 g, 80%). was prepared in a manner similar to that in Example 486 step 5 from 2-chloro-5-iodoquinolin-6-amine and dimethylphosphineoxide. [M+H]⁺=255.1.

### Step 4: N-(6-amino-5-(dimethylphosphoryl)quinolin-2-yl)cyclopropanecarboxamide

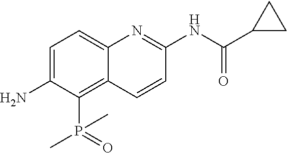 [SMILES: CP(C)(=O)c1c(N)ccc2nc(NC(=O)C3CC3)ccc12]

A mixture of (6-amino-2-chloroquinolin-5-yl)dimethylphosphine oxide (3.5 g, 13.8 mmol), cyclopropanecarboxamide (2.5 g, 69 mmol), Pd(OAc)₂ (132 mg, 0.59 mmol), XantPhos (799 mg, 1.38 mmol), and Cs₂CO₃ (3.83 g, 11.8 mmol) in dioxane (60 mL) was stirred at 100° C. under nitrogen atmosphere for 15 hrs. After cooling to r.t, the reaction mixture was filtered, the filtrate was concentrated in vacuo. The residue was purified by silica gel column (DCM: MeOH=10:1) to afford product (1 g, 56%). [M+H]⁺=304.1.

### Step 5: N-(6-((5-bromo-2-chloropyrimidin-4-yl)amino)-5-(dimethylphosphoryl)quinolin-2-yl)cyclopropanecarboxamide

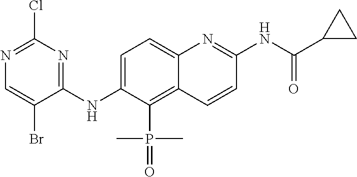 [SMILES: CP(C)(=O)c1c(Nc2nc(Cl)ncc2Br)ccc2nc(NC(=O)C3CC3)ccc12]

To a solution of N-(6-amino-5-(dimethylphosphoryl)quinolin-2-yl)cyclopropanecarboxamide (500 mg, 1.7 mmol) in i-PrOH (50 mL) was added 5-bromo-2,4-dichloropyrimidine (743 mg, 3.4 mmol). DIEA (638 mg, 5.1 mmol) was added to the reaction mixture and the mixture was stirred at 100° C. overnight. The reaction was concentrated in vacuum and the residue was purified by column chromatography (DCM/MeOH=20/1) to afford the product (490 mg, 60%). [M+H]⁺=494.1.

### Step 6: N-(6-((5-bromo-2-((5-ethyl-2-methoxy-4-(4-(piperazin-1-yl)piperidin-1-yl)phenyl)amino)pyrimidin-4-yl)amino)-5-(dimethylphosphoryl)quinolin-2-yl)cyclopropanecarboxamide

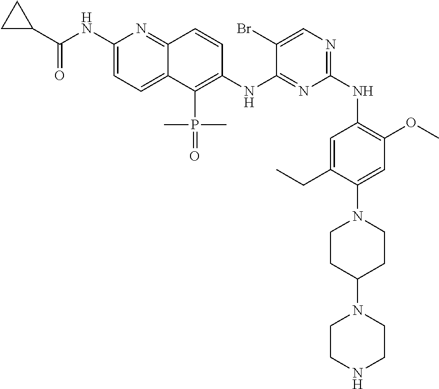 [SMILES: CCc1cc(Nc2ncc(Br)c(Nc3ccc4nc(NC(=O)C5CC5)ccc4c3P(C)(C)=O)n2)c(OC)cc1N1CCC(N2CCNCC2)CC1]

To a stirred solution of N-(6-((5-bromo-2-chloropyrimidin-4-yl)amino)-5-(dimethylphosphoryl)quinolin-2-yl)cyclopropanecarboxamide (50 mg, 0.1 mmol) and tert-butyl 4-(1-(4-amino-2-ethyl-5-methoxyphenyl)piperidin-4-yl)piperazine-1-carboxylate (42 mg, 0.1 mmol) in n-BuOH (40 mL) was added TsOH (52 mg, 0.3 mmol). The resulting mixture was stirred at 100° C. for 16 hours. The reaction mixture was concentrated to dryness, and aq. Na₂CO₃ (0.1M, 10 mL) was poured into the mixture. Then the mixture was extracted with DCM (2×20 mL). The combined organic layer was washed with brine (2×20 mL), dried over Na₂SO₄, filtered and concentrated under vacuum to afford the crude residue, which was purified with silica gel column chromatography (DCM: MeOH=6:1) to give the title product (40 mg, 51%). [M+H]⁺=776.3.

### Step 7: N-(6-((5-bromo-2-((4-(4-(4-((R)-1-(4-((R)-2,6-dioxopiperidin-3-yl)-3,5-difluorophenyl)pyrrolidine-3-carbonyl)piperazin-1-yl)piperidin-1-yl)-5-ethyl-2-methoxyphenyl)amino)pyrimidin-4-yl)amino)-5-(dimethylphosphoryl)uinolin-2-yl)cyclopropanecarboxamide

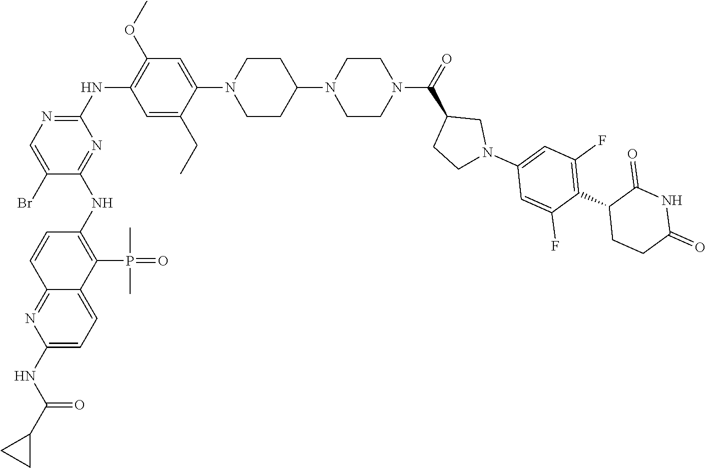 [SMILES: CCc1cc(Nc2ncc(Br)c(Nc3ccc4nc(NC(=O)C5CC5)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

A solution of N-(6-((5-bromo-2-((5-ethyl-2-methoxy-4-(4-(piperazin-1-yl)piperidin-1-yl)phenyl)amino)pyrimidin-4-yl)amino)-5-(dimethylphosphoryl)quinolin-2-yl)cyclopropanecarboxamide (40 mg, 0.05 mmol), (R)-1-(4-((R)-2,6-dioxopiperidin-3-yl)-3,5-difluorophenyl)pyrrolidine-3-carboxylic acid (18 mg, 0.05 mmol), T3P (66 mg, 0.1 mol, 50% in EA) and DIEA (20 mg, 0.15 mmol) in DCM (5 mL) was stirred at r.t. for 1 h. Then the mixture was washed with brine (1×10 mL), dried over Na₂SO₄, filtered and evaporated in vacuum to afford the crude residue, which was purified by silica gel column chromatography (DCM: MeOH=100: 0-10: 1 gradient elution) to give an impure product, which was further purified with prep-HPLC to give the desired product (9.86 mg, 17%). ¹H NMR (500 MHz, DMSO) δ 11.54 (s, 1H), 11.15 (s, 1H), 10.84 (s, 1H), 8.74 (d, J=9.6 Hz, 1H), 8.34 (d, J=9.4 Hz, 1H), 8.23 (d, J=16.9 Hz, 2H), 7.88 (s, 1H), 7.79 (d, J=9.3 Hz, 1H), 7.45 (s, 1H), 6.69 (s, 1H), 6.23 (d, J=12.1 Hz, 2H), 4.02 (dd, J=12.3, 4.8 Hz, 1H), 3.76 (s, 3H), 3.62-3.43 (m, 6H), 3.35 (s, 1H), 3.31-3.22 (m, 3H), 2.90 (d, J=13.8 Hz, 2H), 2.85-2.72 (m, 1H), 2.67-2.51 (m, 6H), 2.37 (s, 1H), 2.30-2.05 (m, 6H), 1.95 (dd, J=17.4, 9.4 Hz, 7H), 1.83 (d, J=10.0 Hz, 2H), 1.56 (d, J=9.3 Hz, 2H), 0.86 (dd, J=10.9, 6.0 Hz, 4H), 0.71 (s, 3H); [M+H]⁺=1096.7.

### Example 588: (R)-3-(4-(2-(4-(1-(4-((5-bromo-4-((5-(dimethylphosphoryl)-2-ethyl-3-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

### Step 1: 3-fluoro-6-nitro-2-vinylquinoline

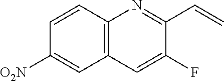 [SMILES: C=Cc1nc2ccc([N+](=O)[O-])cc2cc1F]

The title compound (320 mg, 65%) was prepared in a manner similar to that in Example 488 step 4 from 2-chloro-3-fluoro-6-nitroquinoline and 4,4,5,5-tetramethyl-2-vinyl-1,3,2-dioxaborolane. [M+H]⁺=219.1.

### Step 2: 2-ethyl-3-fluoroquinolin-6-amine

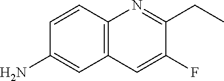

To a solution of 3-fluoro-6-nitro-2-vinylquinoline (320 mg, 1.46 mmol) in MeOH (20 mL)/DCM (10 mL) was added 10% Pd/C (100 mg, 10 wt. %, wet) at 25° C. The flask was evacuated and backfilled with hydrogen three times. The mixture was stirred under hydrogen atmosphere at 25° C. for 2 hours. The mixture was filtered through a pad of celite and washed with MeOH (20 mL). The filtrate was concentrated under vacuum to afford the desired product (255 mg, 91%). [M+H]⁺=191.1.

### Step 3: 2-ethyl-3-fluoro-5-iodoquinolin-6-amine

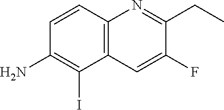

The title compound (400 mg, 95%) was prepared in a manner similar to that in Example 488 step 6 from 2-ethyl-3-fluoroquinolin-6-amine and ICl [M+H]⁺=317.0.

### Step 4: (6-amino-2-ethyl-3-fluoroquinolin-5-yl)dimethylphosphine oxide

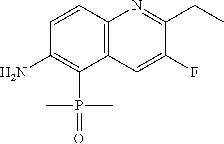 [SMILES: CCc1nc2ccc(N)c(P(C)(C)=O)c2cc1F]

The title compound (320 mg, 95%) was prepared in a manner similar to that in Example 488 step 7 from 2-ethyl-3-fluoro-5-iodoquinolin-6-amine and dimethylphosphine oxide. [M+H]⁺=267.1.

### Step 5: (6-((5-bromo-2-chloropyrimidin-4-yl)amino)-2-ethyl-3-fluoroquinolin-5-yl)dimethylphosphine oxide

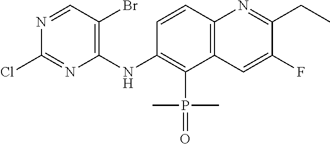 [SMILES: CCc1nc2ccc(Nc3nc(Cl)ncc3Br)c(P(C)(C)=O)c2cc1F]

The title compound (280 mg, 51%) was prepared in a manner similar to that in Example 488 step 8 from (6-amino-2-ethyl-3-fluoroquinolin-5-yl)dimethylphosphine oxide and 5-bromo-2,4-dichloropyrimidine. [M+H]⁺=457.1.

### Step 6: (6-((5-bromo-2-((2-ethoxy-5-ethyl-4-(4-(piperazin-1-yl)piperidin-1-yl)phenyl)amino)pyrimidin-4-yl)amino)-2-ethyl-3-fluoroquinolin-5-yl)dimethylphosphine oxide

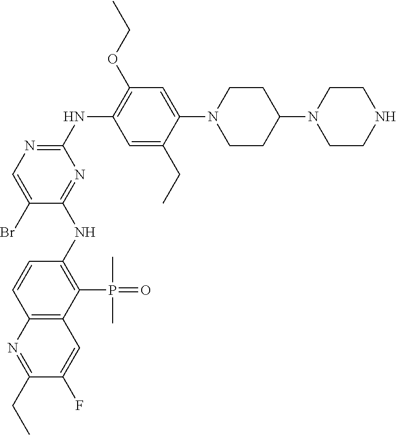 [SMILES: CCOc1cc(N2CCC(N3CCNCC3)CC2)c(CC)cc1Nc1ncc(Br)c(Nc2ccc3nc(CC)c(F)cc3c2P(C)(C)=O)n1]

The title compound (120 mg, 43%) was prepared in a manner similar to that in Example 488 step 9 from (6-((5-bromo-2-chloropyrimidin-4-yl)amino)-2-ethyl-3-fluoroquinolin-5-yl)dimethylphosphine oxide and tert-butyl 4-(1-(4-amino-5-ethoxy-2-ethylphenyl)piperidin-4-yl)piperazine-1-carboxylate. [M+H]⁺=753.2.

### Step 7: (R)-3-(4-(2-(4-(1-(4-((5-bromo-4-((5-(dimethylphosphoryl)-2-ethyl-3-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

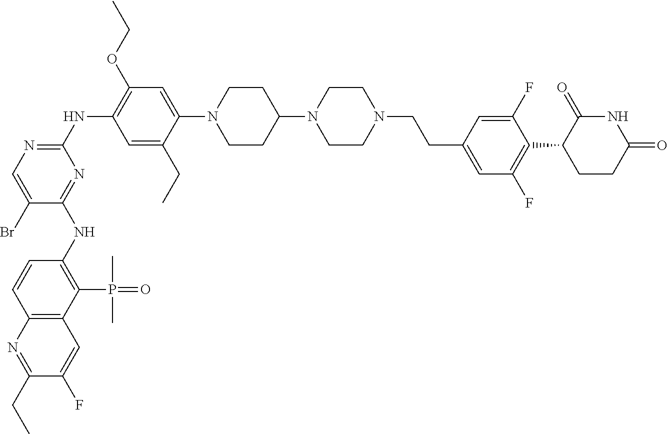 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)c(F)cc3c2P(C)(C)=O)n1]

The title compound (16 mg, 38%) was prepared in a manner similar to that in Example 484 step 15 from (6-((5-bromo-2-((2-ethoxy-5-ethyl-4-(4-(piperazin-1-yl)piperidin-1-yl)phenyl)amino)pyrimidin-4-yl)amino)-2-ethyl-3-fluoroquinolin-5-yl)dimethylphosphine oxide and (R)-2-(4-(2,6-dioxopiperidin-3-yl)-3,5-difluorophenyl)acetaldehyde. ¹H NMR (500 MHz, DMSO) δ 11.27 (s, 1H), 10.95 (s, 1H), 8.64 (d, J=12.4 Hz, 1H), 8.23 (s, 1H), 8.14 (s, 1H), 7.95 (d, J=9.2 Hz, 1H), 7.89 (s, 1H), 7.31 (s, 1H), 7.02 (d, J=10.0 Hz, 2H), 6.69 (s, 1H), 4.20 (dd, J=12.7, 5.2 Hz, 1H), 3.99 (q, J=6.9 Hz, 2H), 2.99 (q, J=7.1 Hz, 2H), 2.93-2.71 (m, 6H), 2.65-2.52 (m, 8H), 2.36 (s, 3H), 2.26 (s, 1H), 2.16-2.10 (m, 3H), 2.02-1.92 (m, 8H), 1.82 (d, J=11.0 Hz, 2H), 1.51 (d, J=9.0 Hz, 2H), 1.33 (t, J=7.5 Hz, 3H), 1.25 (t, J=6.9 Hz, 3H), 0.66 (s, 3H); [M+H]⁺=1004.5.

### Example 589: (R)-3-(4-(2-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

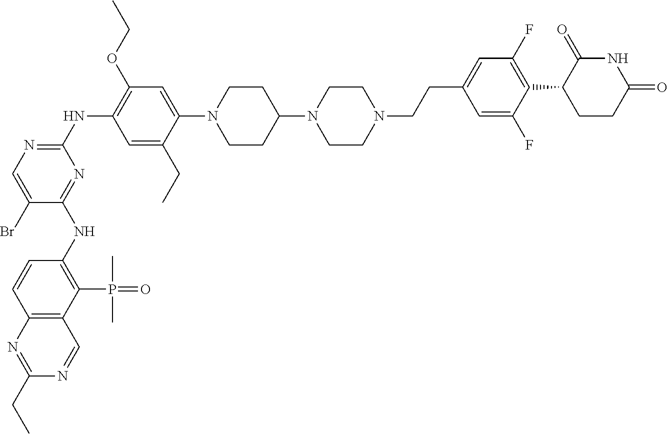 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(CC)ncc3c2P(C)(C)=O)n1]

The title compound was prepared in a procedure similar to that in Example 486. 1H NMR (400 MHz, DMSO) δ 11.53 (s, 1H), 10.88 (s, 1H), 9.79 (s, 1H), 8.38 (s, 1H), 8.16 (s, 1H), 7.98 (s, 1H), 7.78 (d, J=8.8 Hz, 1H), 7.21 (s, 1H), 6.96 (d, J=10.6 Hz, 2H), 6.67 (s, 1H), 4.15-4.11 (m, 1H), 3.68 (s, 3H), 3.00-2.96 (m, 2H), 2.86 (d, J=9.2 Hz, 2H), 2.77-2.74 (m, 1H), 2.70-2.67 (m, 2H), 2.61-2.56 (m, 3H), 2.48-2.45 (m, 7H), 2.40 (s, 2H), 2.22-2.16 (m, 3H), 2.10-2.03 (m, 2H), 1.96 (d, J=13.3 Hz, 7H), 1.77 (d, J=11.4 Hz, 2H), 1.47 (d, J=11.0 Hz, 2H), 1.31 (t, J=7.4 Hz, 3H), 0.68 (s, 3H); [M+H]⁺=973.1.

### Example 590: (R)-3-(4-(3-((4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)methyl)azetidin-1-yl)-2,6-difluorophenyl)piperidine-2,6-dione

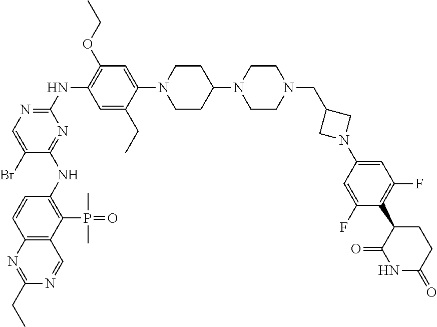 [SMILES: CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3nc(CC)ncc3c2P(C)(C)=O)n1]

The title compound was prepared in a procedure similar to that in Example 486. 1H NMR (400 MHz, DMSO) δ 11.52 (s, 1H), 10.85 (s, 1H), 9.91 (s, 1H), 8.43 (s, 1H), 8.25 (s, 1H), 7.95 (s, 1H), 7.86 (d, J=8.6 Hz, 1H), 7.32 (s, 1H), 6.70 (s, 1H), 6.11 (d, J=11.4 Hz, 2H), 4.03-3.98 (m, 3H), 3.94-3.92 (m, 2H), 3.48 (s, 2H), 3.08-3.03 (m, 2H), 2.91 (s, 3H), 2.81-2.74 (m, 1H), 2.66-2.62 (m, 3H), 2.57-2.52 (m, 5H), 2.36 (s, 2H), 2.19 (s, 2H), 2.03 (d, J=13.4 Hz, 7H), 1.96-1.91 (m, 2H), 1.82 (s, 2H), 1.53 (s, 2H), 1.38 (t, J=7.6 Hz, 3H), 1.29-1.20 (m, 6H), 0.69 (s, 3H); [M+H]⁺=1028.2.

### Example 591: (R)-3-(4-(2-(4-(1-(4-((4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

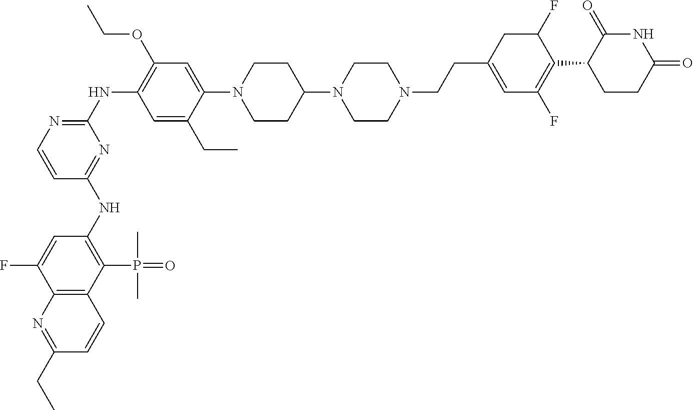 [SMILES: CCOc1cc(N2CCC(N3CCN(CCC4=CC(F)=C([C@H]5CCC(=O)NC5=O)C(F)C4)CC3)CC2)c(CC)cc1Nc1nccc(Nc2cc(F)c3nc(CC)ccc3c2P(C)(C)=O)n1]

A mixture of (R)-3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione (40 mg, 0.04 mmol) and Pd/C(10 mg, 10 wt. %, wet) in MeOH/DCM (10 mL) was stirred in a round bottom flask at 25° C. for 1 hour under hydrogen atmosphere. After filtration, the filtrate was concentrated under reduced pressure. The residue was purified with prep-HPLC (0.1% FA in water: acetonitrile=90:1-50:50 gradient elution) to give the title product (15 mg, 40%). ¹H NMR (500 MHz, DMSO) δ 12.15 (s, 1H), 10.95 (s, 1H), 8.73 (d, J=13.3 Hz, 1H), 8.61 (d, J=9.2 Hz, 1H), 8.06 (d, J=5.6 Hz, 1H), 7.88 (s, 1H), 7.57 (s, 1H), 7.53 (d, J=9.0 Hz, 1H), 7.03 (d, J=10.0 Hz, 2H), 6.75 (s, 1H), 6.08 (d, J=5.6 Hz, 1H), 4.20 (dd, J=12.9, 5.1 Hz, 1H), 4.04 (q, J=7.0 Hz, 2H), 3.02-2.90 (m, 4H), 2.85-2.82 (m, 1H), 2.78-2.71 (m, 2H), 2.69-2.62 (m, 2H), 2.55-2.53 (m, 7H), 2.45-2.42 (m, 6H), 2.32-2.29 (m, 1H), 2.19-2.07 (m, 1H), 2.02-1.98 (d, 7H), 1.86-1.84 (m, 2H), 1.61-1.49 (m, 2H), 1.34-1.24 (m, 6H), 0.91 (t, J=7.2 Hz, 3H); [M+H]⁺=926.5.

### Example 592: 3-(7-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-4-fluoro-2-oxobenzo[d]oxazol-3(2H)-yl)piperidine-2,6-dione

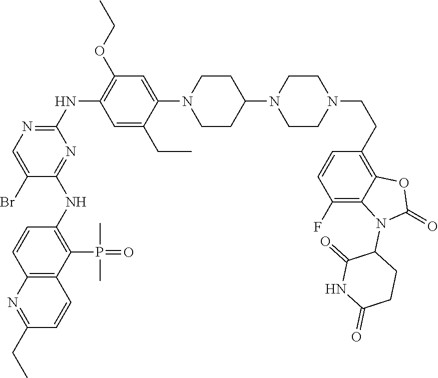 [SMILES: CCOc1cc(N2CCC(N3CCN(CCc4ccc(F)c5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1043.4.

### Example 593: 3-(7-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-5-fluoro-6-methyl-2-oxobenzo[d]oxazol-3(2H)-yl)piperidine-2,6-dione

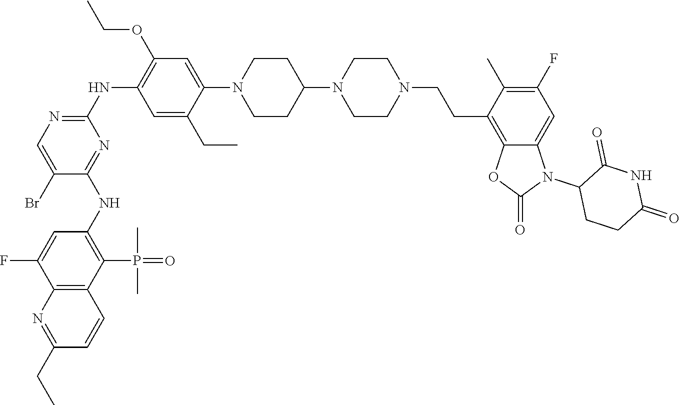 [SMILES: CCOc1cc(N2CCC(N3CCN(CCc4c(C)c(F)cc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1]

The title compound was prepared in a procedure similar to that in Example 492. [M+H]⁺=1057.2.

### Example 594: 3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-3-ethyl-7-fluoro-2-oxo-2,3-dihydro-1H-benzo[d]imidazol-1-yl)piperidine-2,6-dione

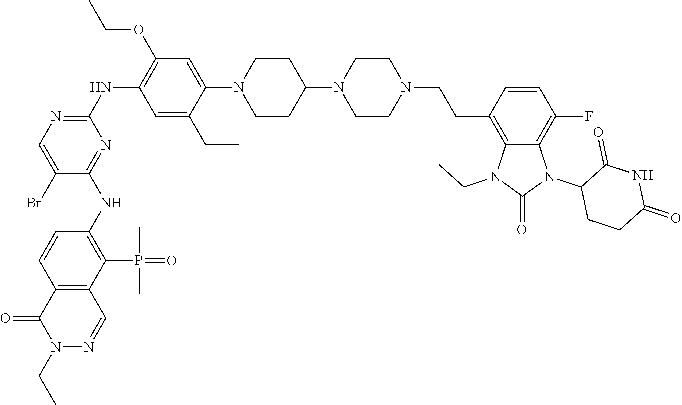 [SMILES: CCOc1cc(N2CCC(N3CCN(CCc4ccc(F)c5c4n(CC)c(=O)n5C4CCC(=O)NC4=O)CC3)CC2)c(CC)cc1Nc1ncc(Br)c(Nc2ccc3c(=O)n(CC)ncc3c2P(C)(C)=O)n1]

The title compound was prepared in a procedure similar to that in Example 318. [M+H]⁺=1069.2.

### Example 595: 3-(7-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-6-fluoro-2-oxobenzo[d]oxazol-3(2H)-yl)piperidine-2,6-dione

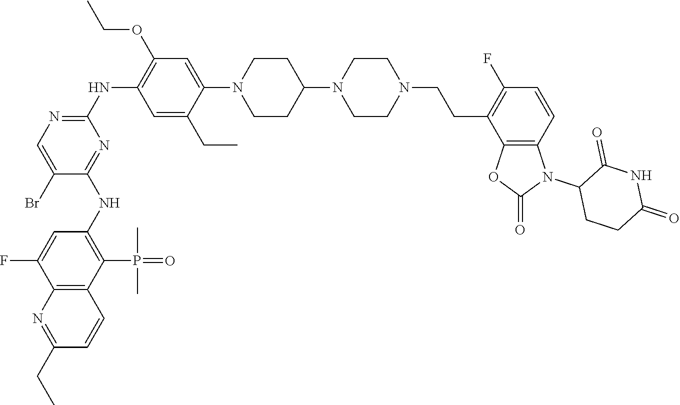 [SMILES: CCOc1cc(N2CCC(N3CCN(CCc4c(F)ccc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1]

The title compound was prepared in a procedure similar to that in Example 492. [M+H]⁺=1043.5.

### Example 596: 3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-3-ethyl-5-fluoro-2-oxo-2,3-dihydro-1H-benzo[d]imidazol-1-yl)piperidine-2,6-dione

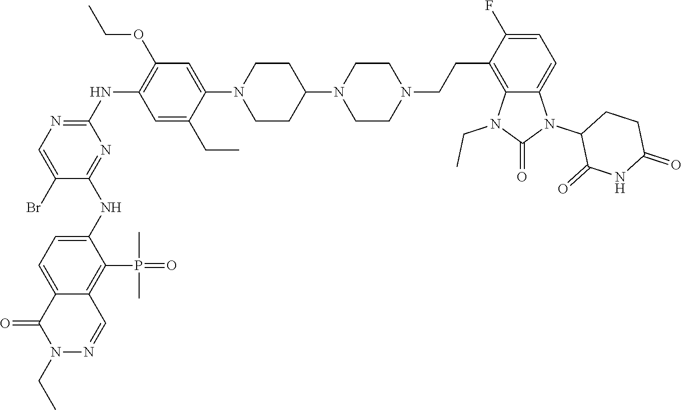 [SMILES: CCOc1cc(N2CCC(N3CCN(CCc4c(F)ccc5c4n(CC)c(=O)n5C4CCC(=O)NC4=O)CC3)CC2)c(CC)cc1Nc1ncc(Br)c(Nc2ccc3c(=O)n(CC)ncc3c2P(C)(C)=O)n1]

The title compound was prepared in a procedure similar to that in Example 318. [M+H]⁺=1070.1.

### Example 597: 3-(7-(3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)propyl)-5-methoxy-2-oxobenzo[d]oxazol-3(2H)-yl)piperidine-2,6-dione

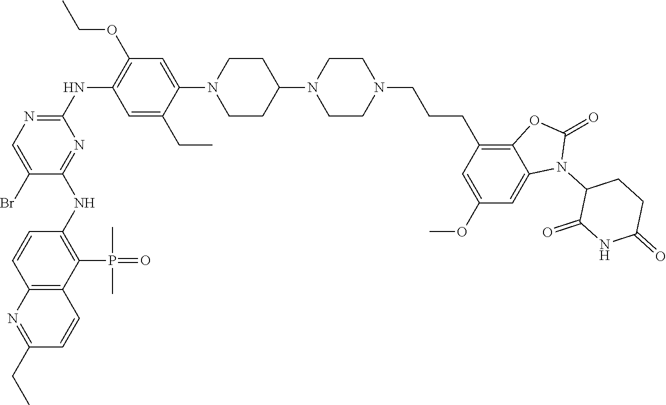 [SMILES: CCOc1cc(N2CCC(N3CCN(CCCc4cc(OC)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1052.1

### Example 598: 3-(7-(3-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)propyl)-5-methoxy-2-oxobenzo[d]oxazol-3(2H)-yl)piperidine-2,6-dione

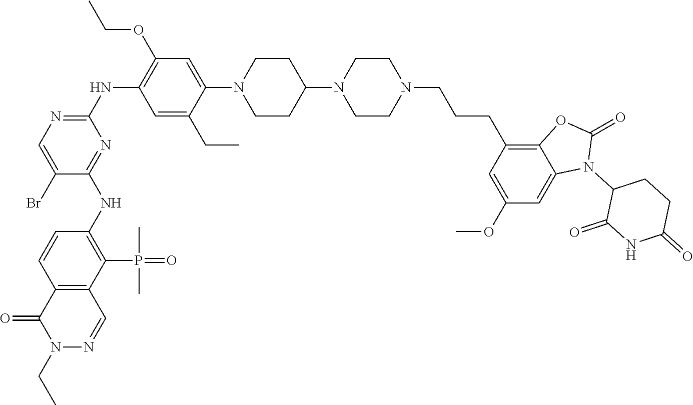 [SMILES: CCOc1cc(N2CCC(N3CCN(CCCc4cc(OC)cc5c4oc(=O)n5C4CCC(=O)NC4=O)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1067.4.

### Example 599: 3-(7-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-5-chloro-2-oxobenzo[d]oxazol-3(2H)-yl)piperidine-2,6-dione

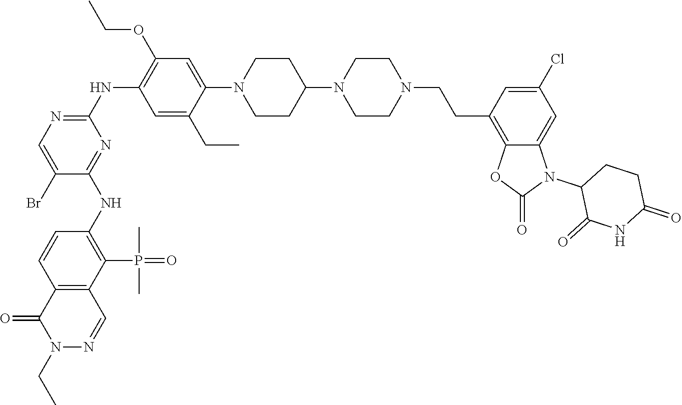 [SMILES: CCOc1cc(N2CCC(N3CCN(CCc4cc(Cl)cc5c4oc(=O)n5C4CCC(=O)NC4=O)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1058.3.

### Example 600: 3-(7-(3-((4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)methyl)azetidin-1-yl)-2-oxobenzo[d]oxazol-3(2H)-yl)piperidine-2,6-dione

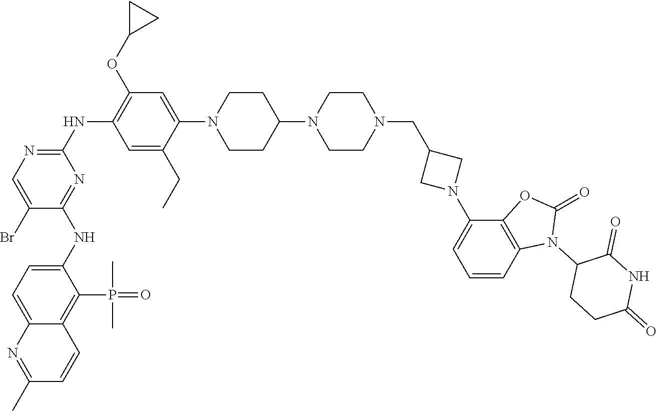 [SMILES: CCc1cc(Nc2ncc(Br)c(Nc3ccc4nc(C)ccc4c3P(C)(C)=O)n2)c(OC2CC2)cc1N1CCC(N2CCN(CC3CN(c4cccc5c4oc(=O)n5C4CCC(=O)NC4=O)C3)CC2)CC1]

The title compound was prepared in a procedure similar to that in Example 492. [M+H]⁺=1046.2.

### Example 601: 3-(6-(2-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)ethyl)-5-fluoro-2-oxobenzo[d]oxazol-3(2H)-yl)piperidine-2,6-dione

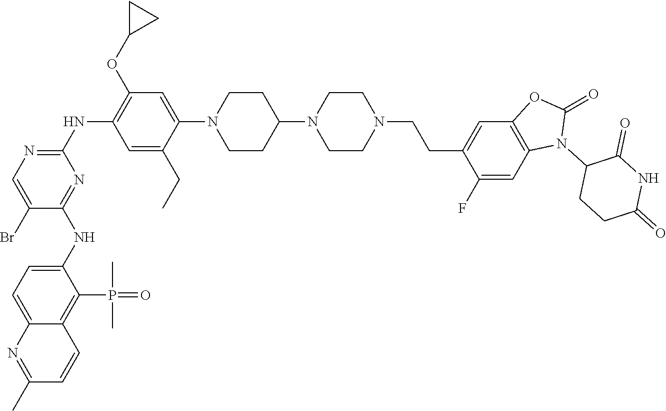 [SMILES: CCc1cc(Nc2ncc(Br)c(Nc3ccc4nc(C)ccc4c3P(C)(C)=O)n2)c(OC2CC2)cc1N1CCC(N2CCN(CCc3cc4oc(=O)n(C5CCC(=O)NC5=O)c4cc3F)CC2)CC1]

The title compound was prepared in a procedure similar to that in Example 492. [M+H]⁺=1023.1.

### Example 602: 3-(4-(2-(4-(1-(4-((5-bromo-4-((5-(dimethylphosphoryl)-2-methylquinolin-6-yl)amino)pyrimidin-2-yl)amino)-5-cyclopropoxy-2-methylphenyl)piperidin-4-yl)piperazin-1-yl)ethyl)-3-methyl-2-oxo-2,3-dihydro-1H-benzo[d]imidazol-1-yl)piperidine-2,6-dione

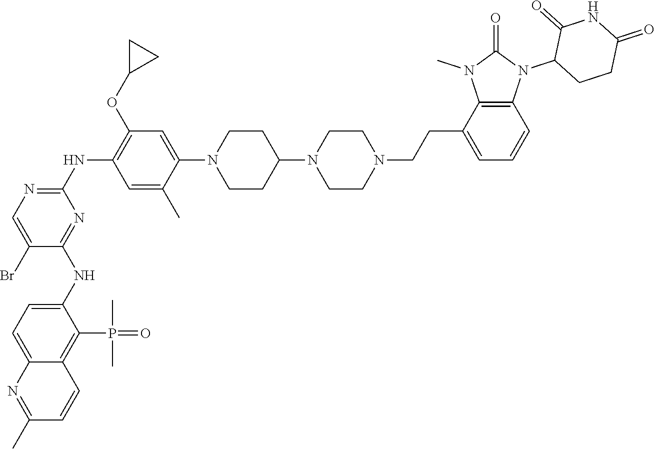 [SMILES: Cc1ccc2c(P(C)(C)=O)c(Nc3nc(Nc4cc(C)c(N5CCC(N6CCN(CCc7cccc8c7n(C)c(=O)n8C7CCC(=O)NC7=O)CC6)CC5)cc4OC4CC4)ncc3Br)ccc2n1]

The title compound was prepared in a procedure similar to that in Example 318. [M+H]⁺=1004.8.

### Example 603: 3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-3-ethyl-7-fluoro-2-oxo-2,3-dihydro-1H-benzo[d]imidazol-1-yl)piperidine-2,6-dione

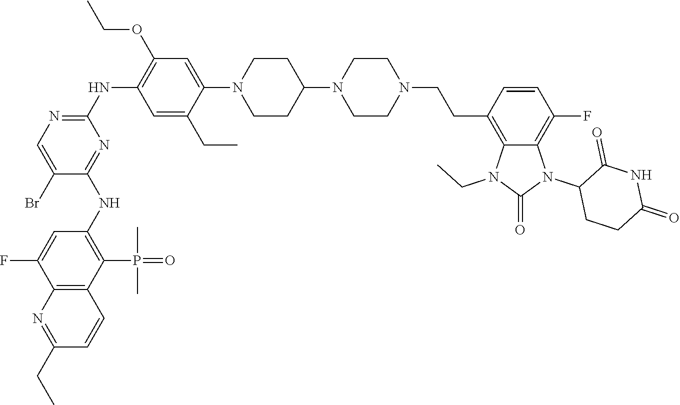 [SMILES: CCOc1cc(N2CCC(N3CCN(CCc4ccc(F)c5c4n(CC)c(=O)n5C4CCC(=O)NC4=O)CC3)CC2)c(CC)cc1Nc1ncc(Br)c(Nc2cc(F)c3nc(CC)ccc3c2P(C)(C)=O)n1]

The title compound was prepared in a procedure similar to that in Example 318. [M+H]⁺=1071.2.

### Example 604: 3-(7-((2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)(methyl)amino)-2-oxobenzo[d]oxazol-3(2H)-yl)piperidine-2,6-dione

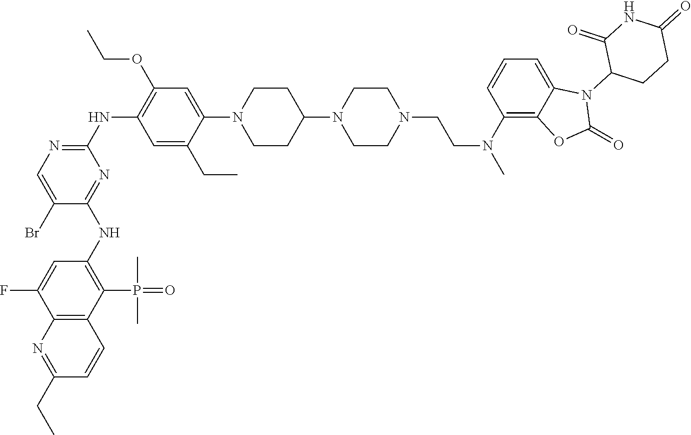 [SMILES: CCOc1cc(N2CCC(N3CCN(CCN(C)c4cccc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1]

The title compound was prepared in a procedure similar to that in Example 492. [M+H]⁺=1053.9.

### Example 605: (R)-3-(4-(3-((4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)methyl)azetidin-1-yl)-2,6-difluorophenyl)piperidine-2,6-dione

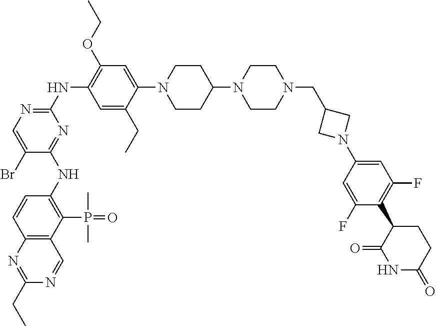 [SMILES: CCOc1cc(N2CCC(N3CCN(CC4CN(c5cc(F)c([C@H]6CCC(=O)NC6=O)c(F)c5)C4)CC3)CC2)c(CC)cc1Nc1ncc(Br)c(Nc2ccc3nc(CC)ncc3c2P(C)(C)=O)n1]

The title compound was prepared in a procedure similar to that in Example 484. [M+H]⁺=1028.2.

### Example 607: 3-(7-(3-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)propyl)-5-fluoro-2-oxobenzo[d]oxazol-3(2H)-yl)piperidine-2,6-dione

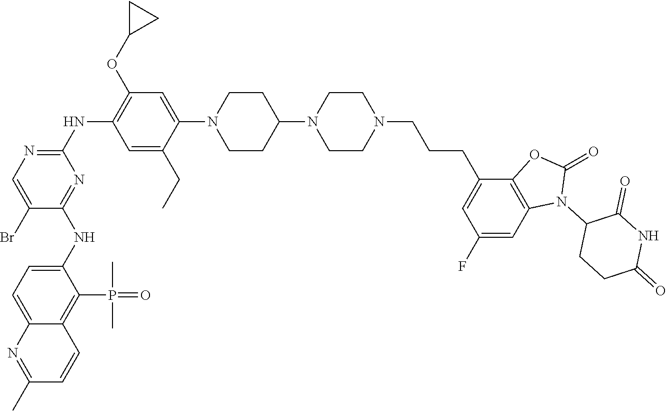 [SMILES: CCc1cc(Nc2ncc(Br)c(Nc3ccc4nc(C)ccc4c3P(C)(C)=O)n2)c(OC2CC2)cc1N1CCC(N2CCN(CCCc3cc(F)cc4c3oc(=O)n4C3CCC(=O)NC3=O)CC2)CC1]

The title compound was prepared in a procedure similar to that in Example 492. [M+H]⁺=1037.2.

### Example 608: 3-(7-(3-((4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)methyl)azetidin-1-yl)-2-oxobenzo[d]oxazol-3(2H)-yl)piperidine-2,6-dione

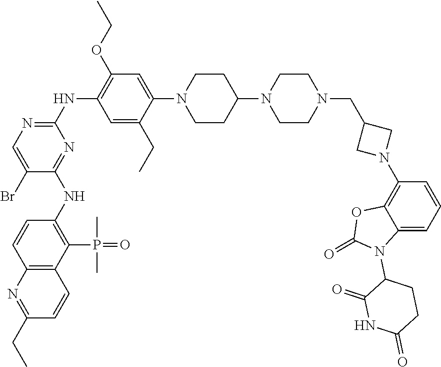 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1049.2.

### Example 609: 3-(6-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-5-fluoro-2-oxobenzo[d]oxazol-3(2H)-yl)piperidine-2,6-dione

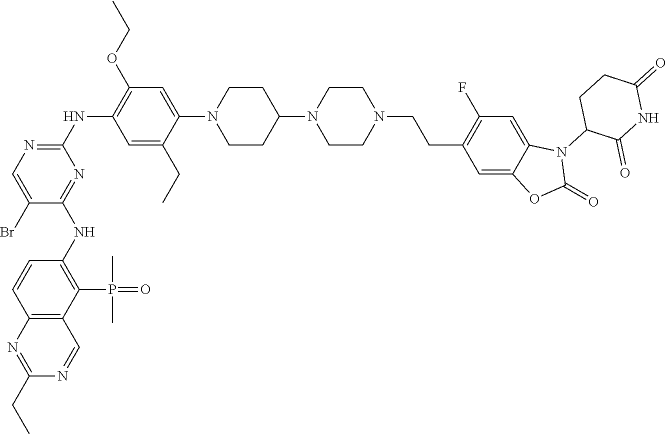 [SMILES: CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2ccc3nc(CC)ncc3c2P(C)(C)=O)n1]

The title compound was prepared in a procedure similar to that in Example 492. [M+H]⁺=1026.2.

### Example 610: 3-(7-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

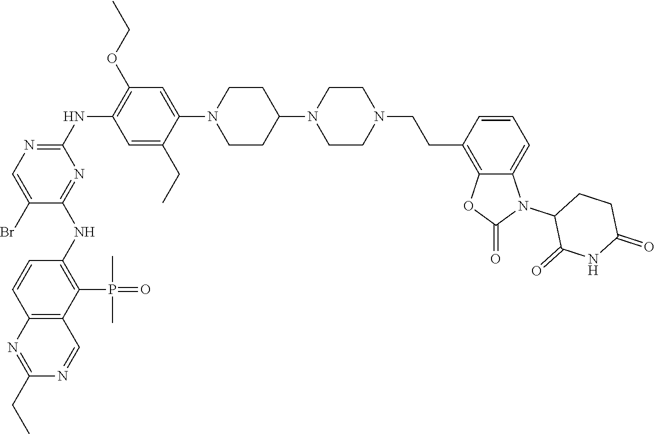 [SMILES: CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3nc(CC)ncc3c2P(C)(C)=O)n1]

The title compound was prepared in a procedure similar to that in Example 492. [M+H]⁺=1008.2.

### Example 611: 3-(7-((R)-3-((4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)methyl)pyrrolidin-1-yl)-2-oxobenzo[d]oxazol-3(2H)-yl)piperidine-2,6-dione

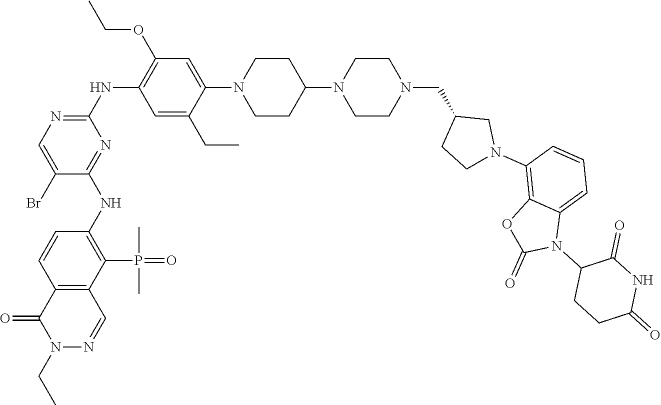 [SMILES: CCOc1cc(N2CCC(N3CCN(C[C@H]4CCN(c5cccc6c5oc(=O)n6C5CCC(=O)NC5=O)C4)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1079.4.

### Example 612: 3-(6-(4-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)piperidin-1-yl)-7-fluoro-2-oxobenzo[d]oxazol-3(2H)-yl)piperidine-2,6-dione

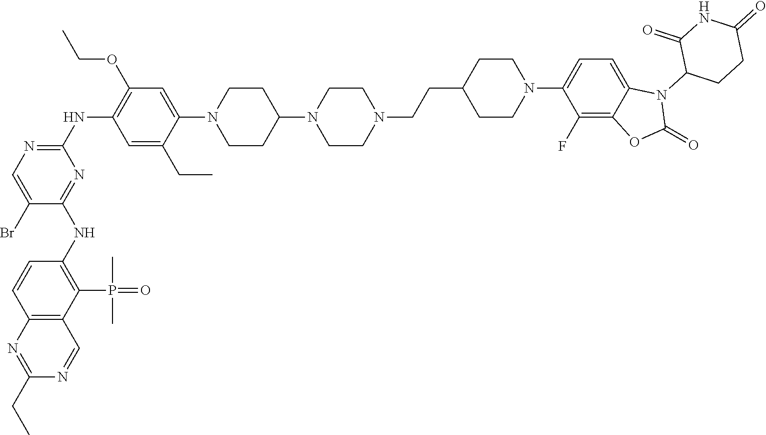 [SMILES: CCOc1cc(N2CCC(N3CCN(CCC4CCN(c5ccc6c(oc(=O)n6C6CCC(=O)NC6=O)c5F)CC4)CC3)CC2)c(CC)cc1Nc1ncc(Br)c(Nc2ccc3nc(CC)ncc3c2P(C)(C)=O)n1]

The title compound was prepared in a procedure similar to that in Example 492. [M+H]⁺=1108.9.

### Example 613: 3-(6-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-5-fluoro-7-methyl-2-oxobenzo[d]oxazol-3(2H)-yl)piperidine-2,6-dione

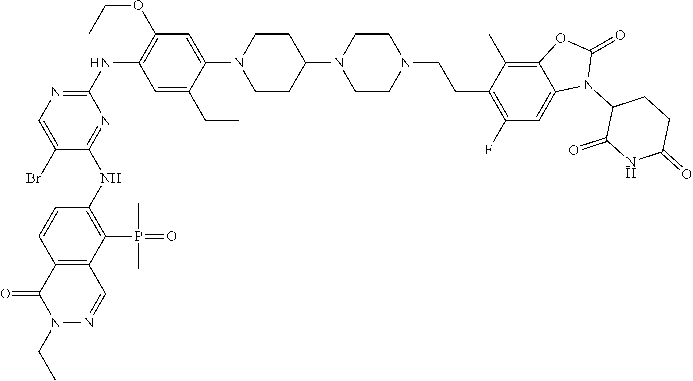 [SMILES: CCOc1cc(N2CCC(N3CCN(CCc4c(F)cc5c(oc(=O)n5C5CCC(=O)NC5=O)c4C)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1055.9.

### Example 614: 3-(7-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-6-fluoro-2-oxobenzo[d]oxazol-3(2H)-yl)piperidine-2,6-dione

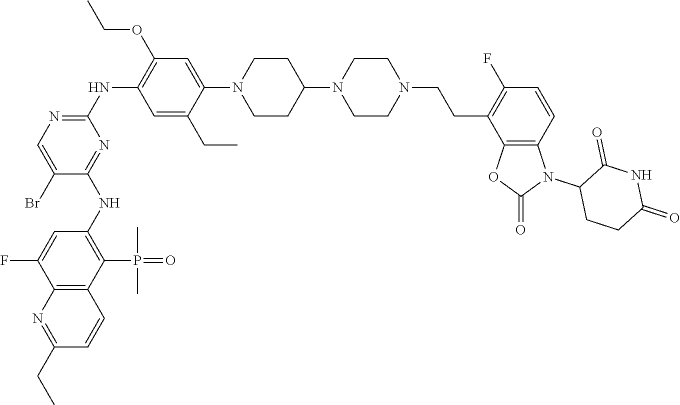 [SMILES: CCOc1cc(N2CCC(N3CCN(CCc4c(F)ccc5c4oc(=O)n5C4CCC(=O)NC4=O)CC3)CC2)c(CC)cc1Nc1ncc(Br)c(Nc2cc(F)c3nc(CC)ccc3c2P(C)(C)=O)n1]

The title compound was prepared in a procedure similar to that in Example 492. [M+H]⁺=1043.5.

### Example 615: 3-(7-(3-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)propyl)-2-oxobenzo[d]oxazol-3(2H)-yl)piperidine-2,6-dione

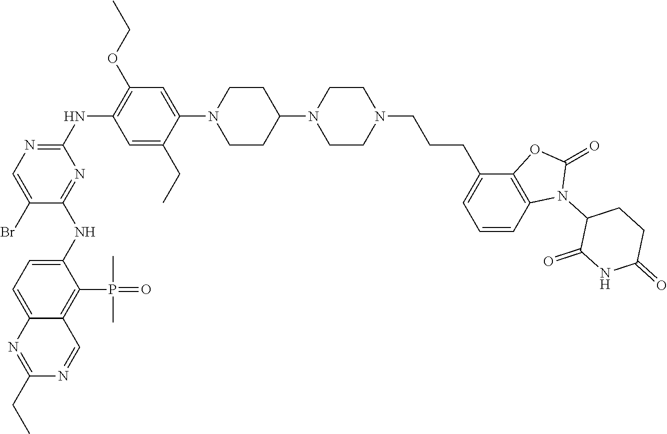 [SMILES: CCOc1cc(N2CCC(N3CCN(CCCc4cccc5c4oc(=O)n5C4CCC(=O)NC4=O)CC3)CC2)c(CC)cc1Nc1ncc(Br)c(Nc2ccc3nc(CC)ncc3c2P(C)(C)=O)n1]

The title compound was prepared in a procedure similar to that in Example 492. [M+H]⁺=1021.9.

### Example 616: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-2-oxo-5-(trifluoromethyl)benzo[d]oxazol-3(2H)-yl)piperidine-2,6-dione

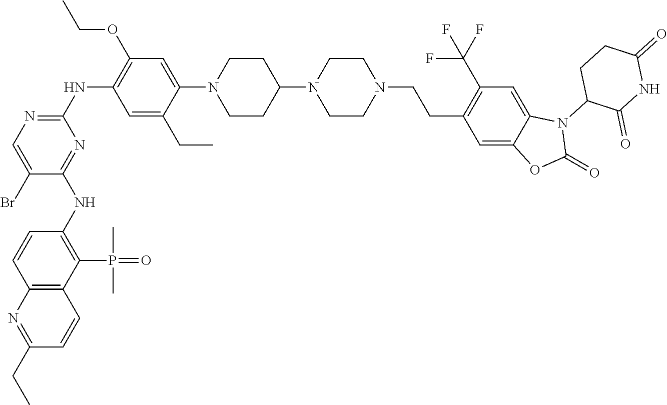 [SMILES: CCOc1cc(N2CCC(N3CCN(CCc4cc5oc(=O)n(C6CCC(=O)NC6=O)c5cc4C(F)(F)F)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1075.2

### Example 617: 3-(7-(3-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)propyl)-5-fluoro-2-oxobenzo[d]oxazol-3(2H)-yl)piperidine-2,6-dione

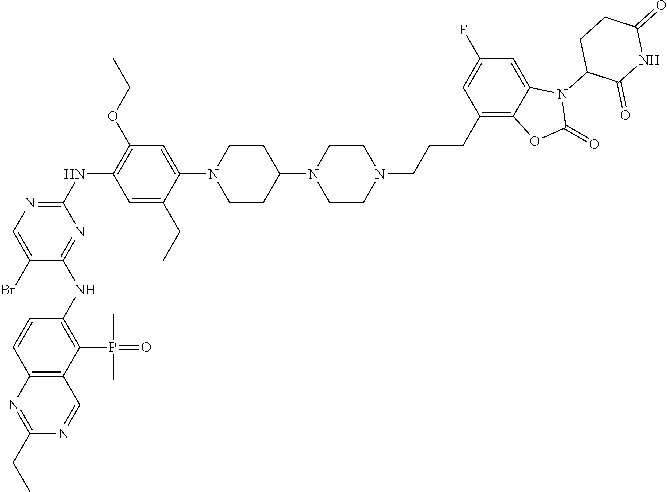 [SMILES: CCOc1cc(N2CCC(N3CCN(CCCc4cc(F)cc5c4oc(=O)n5C4CCC(=O)NC4=O)CC3)CC2)c(CC)cc1Nc1ncc(Br)c(Nc2ccc3nc(CC)ncc3c2P(C)(C)=O)n1]

The title compound was prepared in a procedure similar to that in Example 492. [M+H]⁺=1040.2.

### Example 618: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-methoxy-2-oxobenzo[d]oxazol-3(2H)-yl)piperidine-2,6-dione

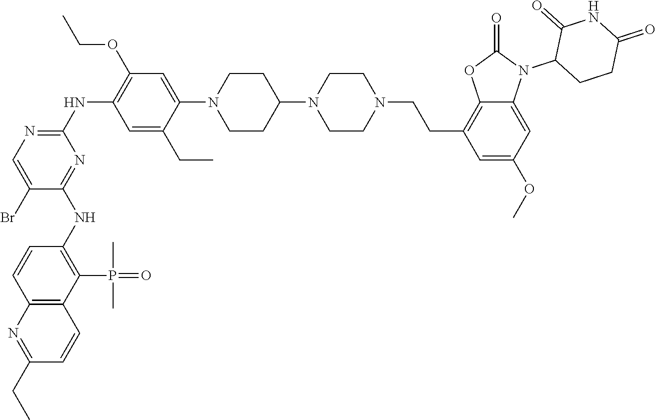 [SMILES: CCOc1cc(N2CCC(N3CCN(CCc4cc(OC)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1037.4.

### Example 619: 3-(7-(2-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethoxy)ethyl)-2-oxobenzod[d]oxazol-3(2H)-yl)piperidine-2,6-dione

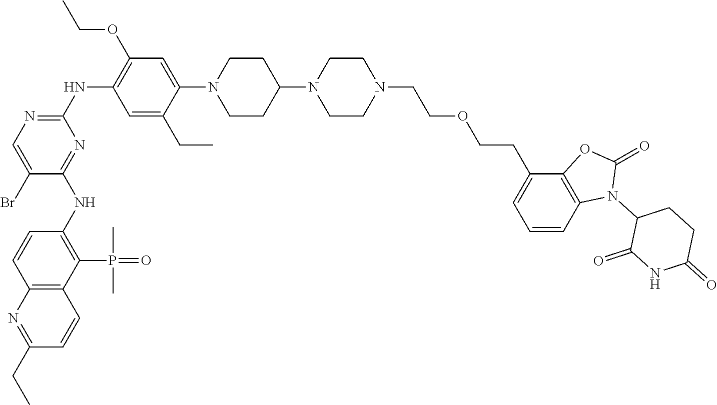 [SMILES: CCOc1cc(N2CCC(N3CCN(CCOCCc4cccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1051.5.

### Example 620: 3-(7-(3-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)propyl)-5-fluoro-2-oxobenzo[d]oxazol-3(2H)-yl)piperidine-2,6-dione

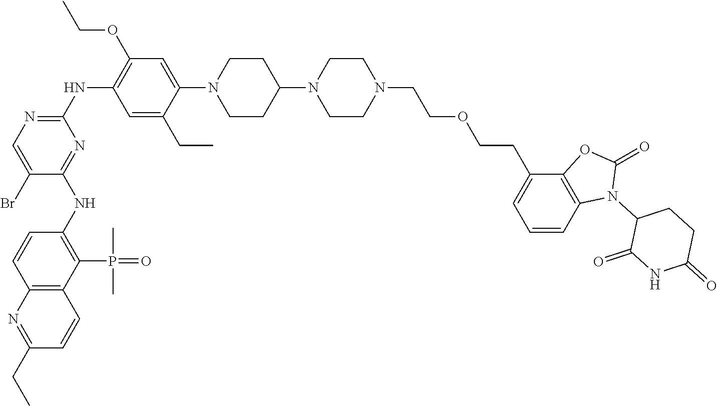 [SMILES: CCOc1cc(N2CCC(N3CCN(CCOCCc4cccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1057.8.

### Example 621: 3-(7-(4-((4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)methyl)piperidin-1-yl)-5-fluoro-2-oxobenzo[d]oxazol-3(2H)-yl)piperidine-2,6-dione

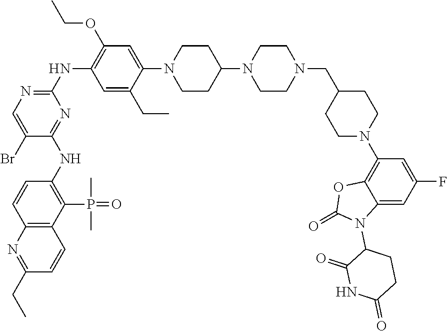 [SMILES: CCOc1cc(N2CCC(N3CCN(CC4CCN(c5cc(F)cc6c5oc(=O)n6C5CCC(=O)NC5=O)CC4)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1095.2.

### Example 622: 3-(4-(3-((4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)methyl)azetidin-1-yl)-3-methyl-2-oxo-2,3-dihydro-1H-benzo[d]imidazol-1-yl)piperidine-2,6-dione

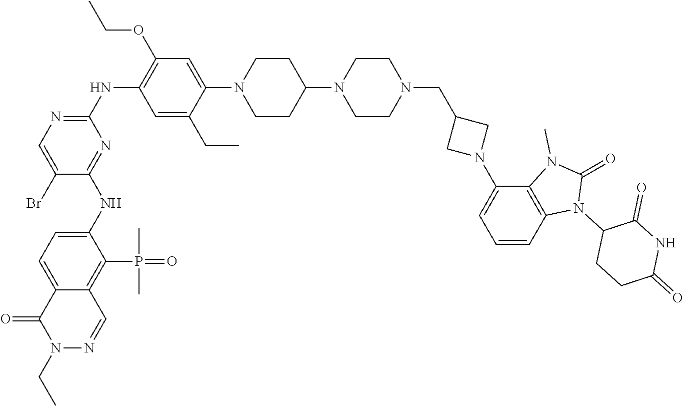 [SMILES: CCOc1cc(N2CCC(N3CCN(CC4CN(c5cccc6c5n(C)c(=O)n6C5CCC(=O)NC5=O)C4)CC3)CC2)c(CC)cc1Nc1ncc(Br)c(Nc2ccc3c(=O)n(CC)ncc3c2P(C)(C)=O)n1]

The title compound was prepared in a procedure similar to that in Example 318. [M+H]⁺=1079.1.

### Example 623: 3-(7-((S)-3-((4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)methyl)pyrrolidin-1-yl)-2-oxobenzo[d]oxazol-3(2H)-yl)piperidine-2,6-dione

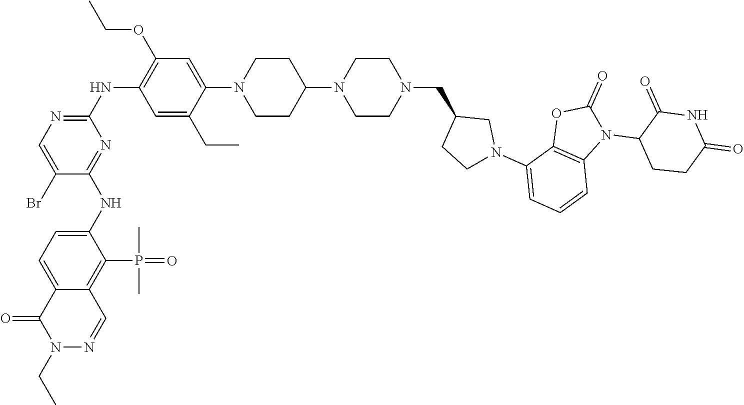 [SMILES: CCOc1cc(N2CCC(N3CCN(C[C@@H]4CCN(c5cccc6c5oc(=O)n6C5CCC(=O)NC5=O)C4)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1080.2.

### Example 624: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-7-chloro-2-oxobenzo[d]oxazol-3(2H)-yl)piperidine-2,6-dione

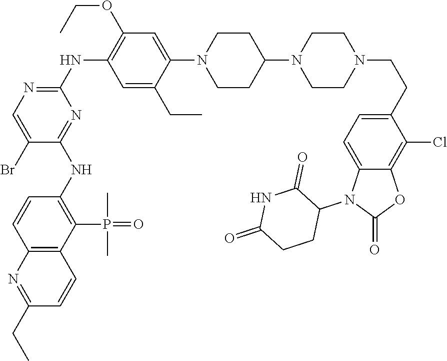 [SMILES: CCOc1cc(N2CCC(N3CCN(CCc4ccc5c(oc(=O)n5C5CCC(=O)NC5=O)c4Cl)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1041.6.

### Example 625: 3-(7-(3-(3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)-3-oxopropyl)azetidin-1-yl)-2-oxobenzo[d]oxazol-3(2H)-yl)piperidine-2,6-dione

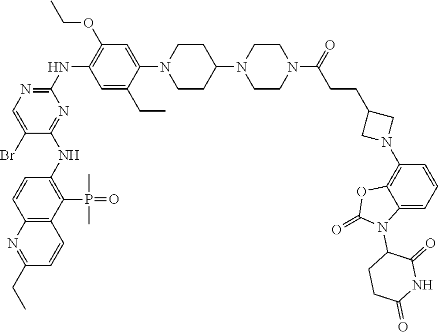 [SMILES: CCOc1cc(N2CCC(N3CCN(C(=O)CCC4CN(c5cccc6c5oc(=O)n6C5CCC(=O)NC5=O)C4)CC3)CC2)c(CC)cc1Nc1ncc(Br)c(Nc2ccc3nc(CC)ccc3c2P(C)(C)=O)n1]

The title compound was prepared in a procedure similar to that in Example 447. [M+H]⁺=1090.7.

### Example 626: 3-(6-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-5-chloro-2-oxobenzo[d]oxazol-3(2H)-yl)piperidine-2,6-dione

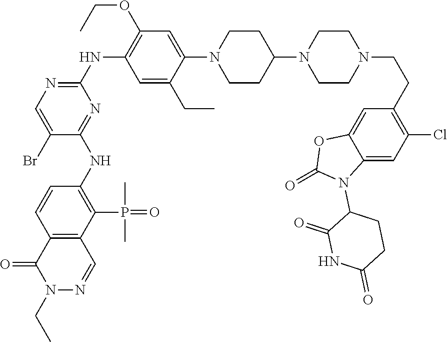 [SMILES: CCOc1cc(N2CCC(N3CCN(CCc4cc5oc(=O)n(C6CCC(=O)NC6=O)c5cc4Cl)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1058.9.

### Example 627: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-chloro-2-oxobenzo[d]oxazol-3(2H)-yl)piperidine-2,6-dione

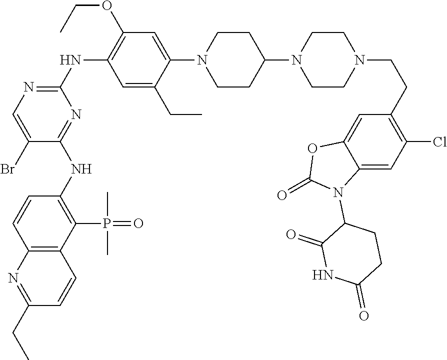 [SMILES: CCOc1cc(N2CCC(N3CCN(CCc4cc5oc(=O)n(C6CCC(=O)NC6=O)c5cc4Cl)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1040.9.

### Example 628: 3-(6-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-5-fluoro-2-oxobenzo[d]oxazol-3(2H)-yl)piperidine-2,6-dione

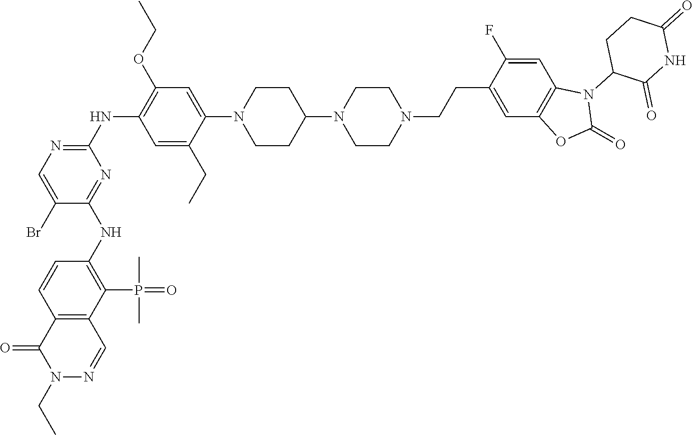 [SMILES: CCOc1cc(N2CCC(N3CCN(CCc4cc5oc(=O)n(C6CCC(=O)NC6=O)c5cc4F)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1042.7.

### Example 629: 3-(6-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-5,7-difluoro-2-oxobenzo[d]oxazol-3(2H)-yl)piperidine-2,6-dione

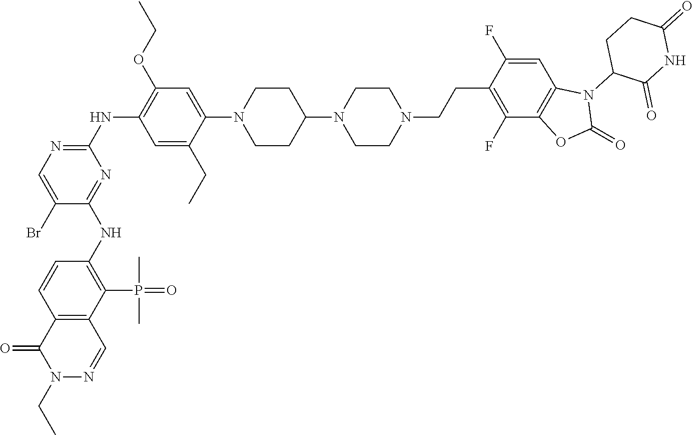 [SMILES: CCOc1cc(N2CCC(N3CCN(CCc4c(F)cc5c(oc(=O)n5C5CCC(=O)NC5=O)c4F)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1060.7.

### Example 630: 3-(7-(3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)propyl)-2-oxobenzo[d]oxazol-3(2H)-yl)piperidine-2,6-dione

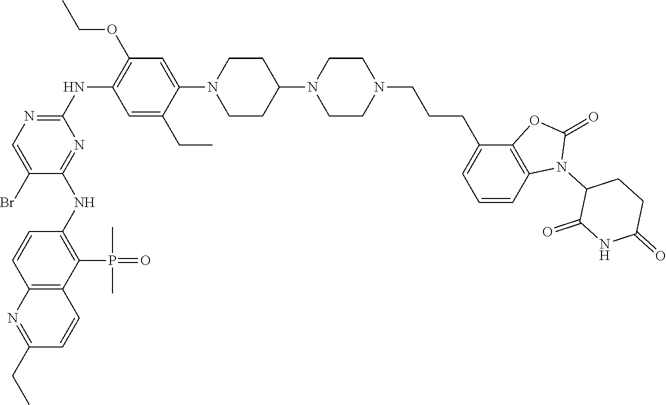 [SMILES: CCOc1cc(N2CCC(N3CCN(CCCc4cccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1021.5.

### Example 631: 3-(7-(2-(4-(1-(4-((5-bromo-4-((5-(dimethylphosphoryl)-2-ethylquinolin-6-yl)amino)pyrimidin-2-yl)amino)-2-cyclopropyl-5-methoxyphenyl)piperidin-4-yl)piperazin-1-yl)ethyl)-2-oxobenzo[d]oxazol-3(2H)-yl)piperidine-2,6-dione

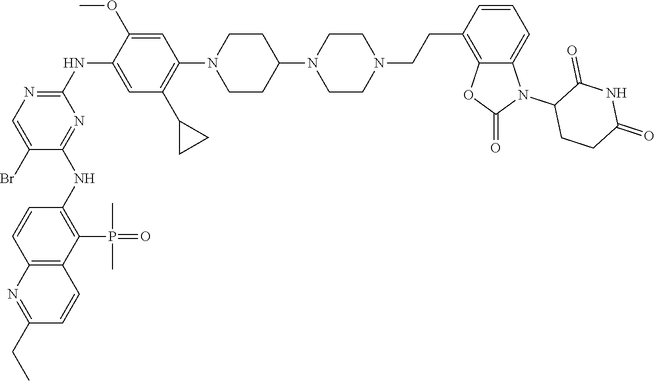 [SMILES: CCc1ccc2c(P(C)(C)=O)c(Nc3nc(Nc4cc(C5CC5)c(N5CCC(N6CCN(CCc7cccc8c7oc(=O)n8C7CCC(=O)NC7=O)CC6)CC5)cc4OC)ncc3Br)ccc2n1]

The title compound was prepared in a procedure similar to that in Example 492. [M+H]⁺=1005.7.

### Example 632: 3-(7-(4-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)butyl)-5-fluoro-2-oxobenzo[d]oxazol-3(2H)-yl)piperidine-2,6-dione

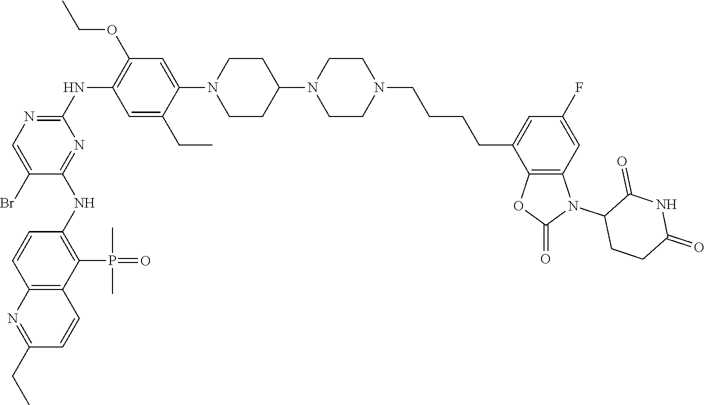 [SMILES: CCOc1cc(N2CCC(N3CCN(CCCCc4cc(F)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1053.4.

### Example 633: 3-(6-(2-(4-(1-(4-((5-bromo-4-((2-(cyclopropylamino)-5-(dimethylphosphoryl)quinolin-6-yl)amino)pyrimidin-2-yl)amino)-2-ethyl-5-methoxyphenyl)piperidin-4-yl)piperazin-1-yl)ethyl)-2-oxobenzo[d]oxazol-3(2H)-yl)piperidine-2,6-dione

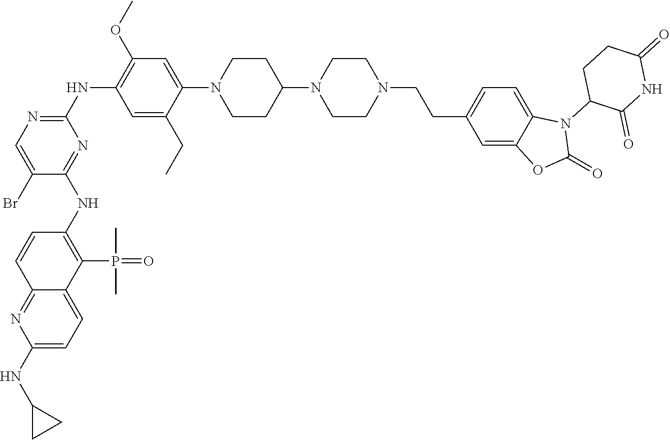 [SMILES: CCc1cc(Nc2ncc(Br)c(Nc3ccc4nc(NC5CC5)ccc4c3P(C)(C)=O)n2)c(OC)cc1N1CCC(N2CCN(CCc3ccc4c(c3)oc(=O)n4C3CCC(=O)NC3=O)CC2)CC1]

The title compound was prepared in a procedure similar to that in Example 492. [M+H]⁺=1020.4.

### Example 634: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-6-methyl-2-oxobenzo[d]oxazol-3(2H)-yl)piperidine-2,6-dione

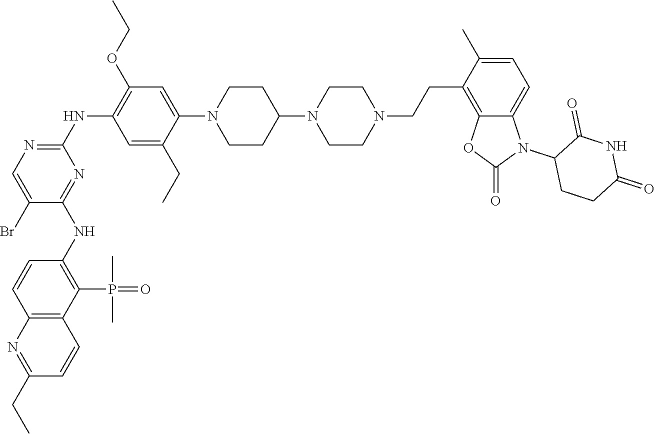 [SMILES: CCOc1cc(N2CCC(N3CCN(CCc4c(C)ccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1021.5.

### Example 635: 3-(6-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5,7-difluoro-2-oxobenzo[d]oxazol-3(2H)-yl)piperidine-2,6-dione

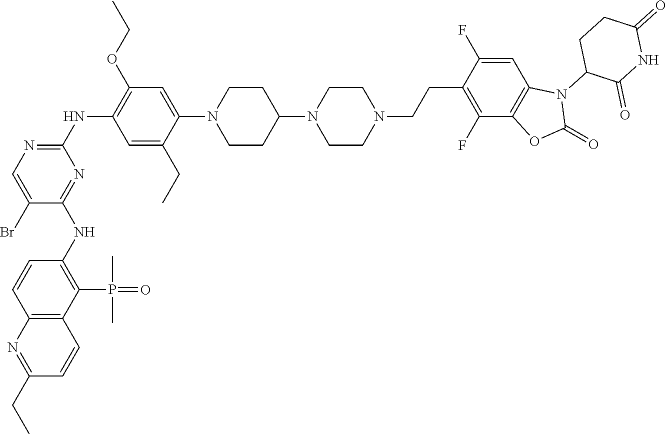 [SMILES: CCOc1cc(N2CCC(N3CCN(CCc4c(F)cc5c(oc(=O)n5C5CCC(=O)NC5=O)c4F)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1043.7.

### Example 636: 3-(7-(3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)propyl)-5-methyl-2-oxobenzo[d]oxazol-3(2H)-yl)piperidine-2,6-dione

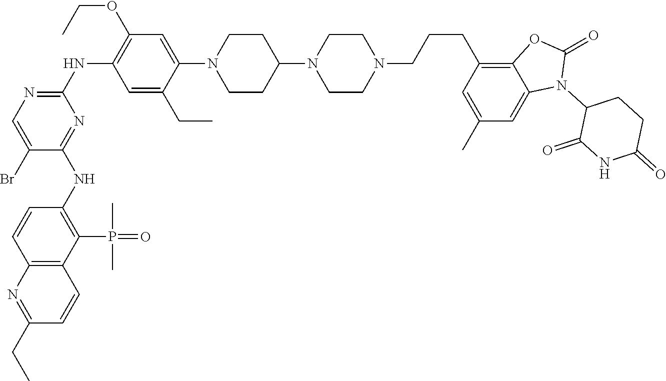 [SMILES: CCOc1cc(N2CCC(N3CCN(CCCc4cc(C)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1034.9.

### Example 637: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)-2-oxoethoxy)-3-methyl-2-oxo-2,3-dihydro-1H-benzo[d]imidazol-1-yl)piperidine-2,6-dione

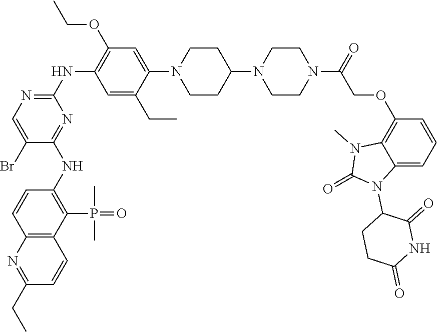 [SMILES: CCOc1cc(N2CCC(N3CCN(C(=O)COc4cccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 447. [M+H]⁺=1049.9.

### Example 639: (R)-3-(4-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)-2-oxoethyl)piperazin-1-yl)-2,6-difluorophenyl)piperidine-2,6-dione

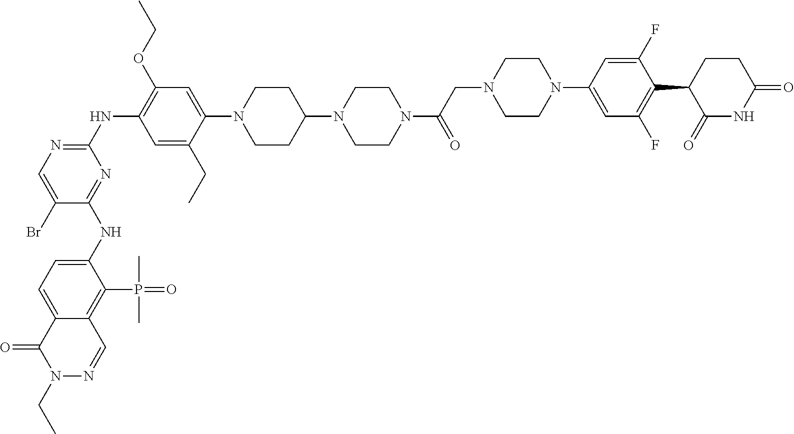 [SMILES: CCOc1cc(N2CCC(N3CCN(C(=O)CN4CCN(c5cc(F)c([C@H]6CCC(=O)NC6=O)c(F)c5)CC4)CC3)CC2)c(CC)cc1Nc1ncc(Br)c(Nc2ccc3c(=O)n(CC)ncc3c2P(C)(C)=O)n1]

The title compound was prepared in a procedure similar to that in Example 488. [M+H]⁺=1102.1.

### Example 640: 3-(5-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)piperidin-1-yl)-3-ethyl-7-fluoro-2-oxo-2,3-dihydro-1H-benzo[d]imidazol-1-yl)piperidine-2, 6-dione

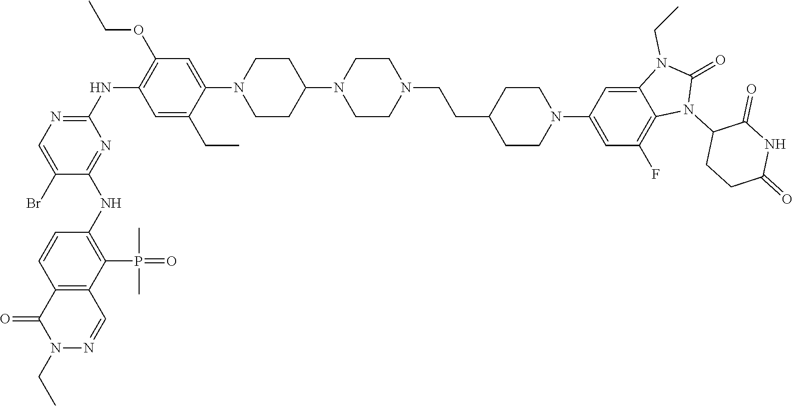 [SMILES: CCOc1cc(N2CCC(N3CCN(CCC4CCN(c5cc(F)c6c(c5)n(CC)c(=O)n6C5CCC(=O)NC5=O)CC4)CC3)CC2)c(CC)cc1Nc1ncc(Br)c(Nc2ccc3c(=O)n(CC)ncc3c2P(C)(C)=O)n1]

The title compound was prepared in a procedure similar to that in Example 318. [M+H]⁺=1151.9.

### Example 641: 3-(4-(2-((S)-4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)-2-(hydroxymethyl)piperazin-1-yl)ethyl)-3,3-dimethyl-2-oxoindolin-1-yl)piperidine-2,6-dione

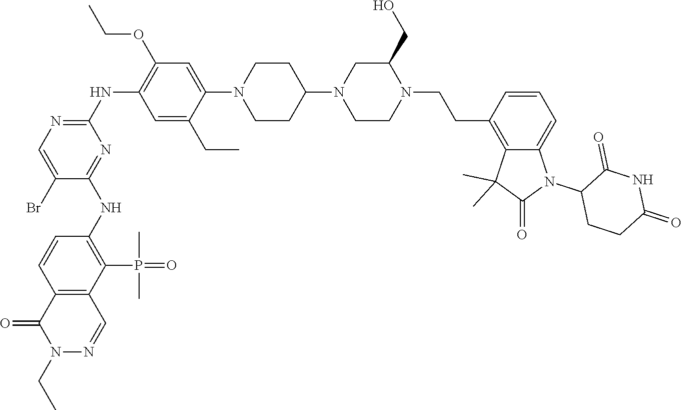 [SMILES: CCOc1cc(N2CCC(N3CCN(CCc4cccc5c4C(C)(C)C(=O)N5C4CCC(=O)NC4=O)[C@H](CO)C3)CC2)c(CC)cc1Nc1ncc(Br)c(Nc2ccc3c(=O)n(CC)ncc3c2P(C)(C)=O)n1]

The title compound was prepared in a procedure similar to that in Example 413. [M+H]⁺=1080.7.

### Example 642: 3-(4-(2-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)ethyl)-3-methyl-2-oxo-2,3-dihydro-1H-benzo[d]imidazol-1-yl)piperidine-2,6-dione

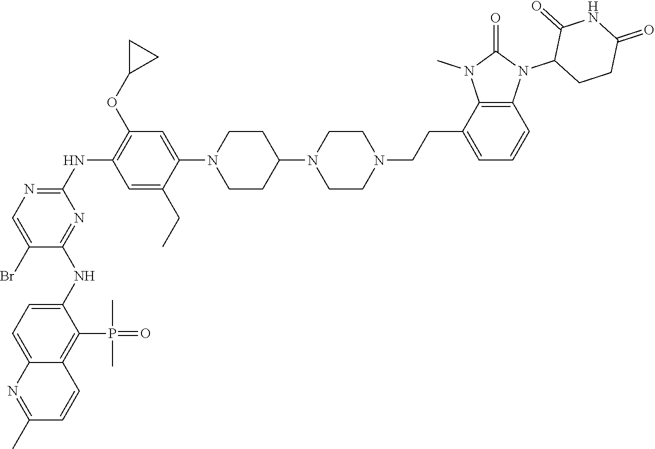 [SMILES: CCc1cc(Nc2ncc(Br)c(Nc3ccc4nc(C)ccc4c3P(C)(C)=O)n2)c(OC2CC2)cc1N1CCC(N2CCN(CCc3cccc4c3n(C)c(=O)n4C3CCC(=O)NC3=O)CC2)CC1]

The title compound was prepared in a procedure similar to that in Example 318. [M+H]⁺=1017.9.

### Example 643: 3-(4-(2-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-3-ethyl-2-oxo-2,3-dihydro-1H-benzo[d]imidazol-1-yl)piperidine-2,6-dione

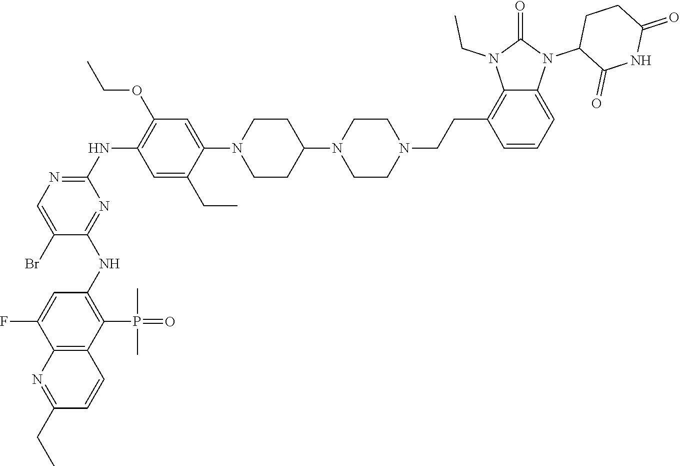 [SMILES: CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2cc(F)c3nc(CC)ccc3c2P(C)(C)=O)n1]

The title compound was prepared in a procedure similar to that in Example 318. ¹H NMR (500 MHz, DMSO) δ 12.03 (s, 1H), 11.03 (s, 1H), 8.50 (d, J=8.4 Hz, 1H), 8.25 (d, J=8.7 Hz, 1H), 8.16-8.14 (m, 1H), 7.97 (s, 1H), 7.47 (d, J=9.0 Hz, 1H), 7.33 (s, 1H), 6.96-6.82 (m, 3H), 6.65 (s, 1H), 5.32-5.28 (m, 1H), 3.95-3.93 (m, 4H), 2.96-2.77 (m, 8H), 2.70-2.46 (m, 13H), 2.30-2.20 (m, 3H), 1.93 (d, J=13.3 Hz, 7H), 1.78-1.75 (m, 2H), 1.50-1.43 (m, 2H), 1.28-1.13 (m, 9H), 0.68 (s, 3H). [M+H]⁺=1052.7.

### Example 644: 3-(7-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

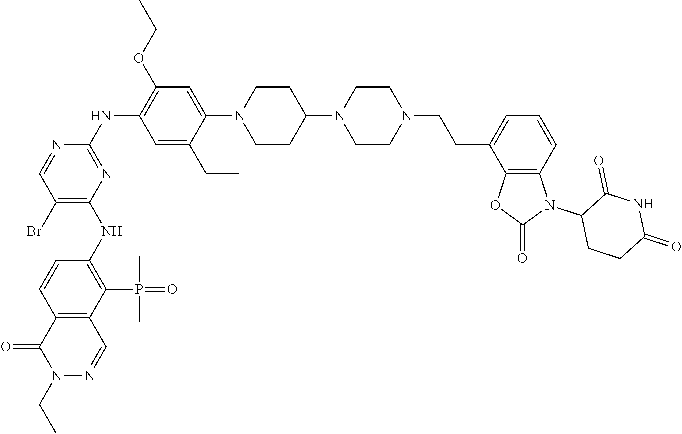 [SMILES: CCOc1cc(N2CCC(N3CCN(CCc4cccc5c4oc(=O)n5C4CCC(=O)NC4=O)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1024.5.

### Example 645: 3-(7-(3-((4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)methyl)azetidin-1-yl)-2-oxobenzo[d]oxazol-3(2H)-yl)piperidine-2,6-dione

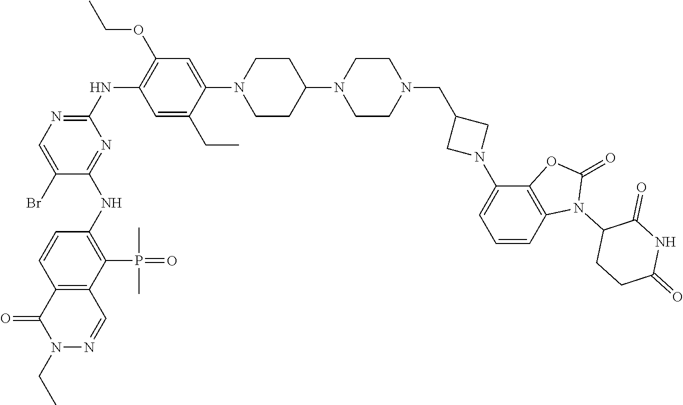 [SMILES: CCOc1cc(N2CCC(N3CCN(CC4CN(c5cccc6c5oc(=O)n6C5CCC(=O)NC5=O)C4)CC3)CC2)c(CC)cc1Nc1ncc(Br)c(Nc2ccc3c(=O)n(CC)ncc3c2P(C)(C)=O)n1]

The title compound was prepared in a procedure similar to that in Example 492. [M+H]⁺=1065.7.

### Example 646: 3-(6-(2-(4-(1-(4-((5-bromo-4-((5-(dimethylphosphoryl)-2-ethylquinazolin-6-yl)amino)pyrimidin-2-yl)amino)-2-ethyl-5-methoxyphenyl)piperidin-4-yl)piperazin-1-yl)-2-oxoethyl)-2-oxobenzo[d]oxazol-3(2H)-yl)piperidine-2,6-dione

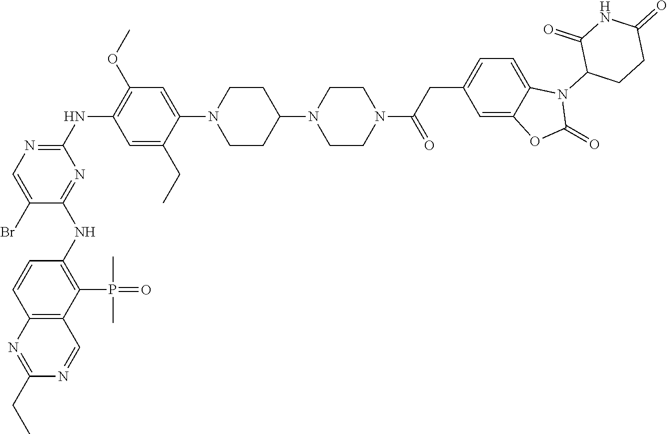 [SMILES: CCc1ncc2c(P(C)(C)=O)c(Nc3nc(Nc4cc(CC)c(N5CCC(N6CCN(C(=O)Cc7ccc8c(c7)oc(=O)n8C7CCC(=O)NC7=O)CC6)CC5)cc4OC)ncc3Br)ccc2n1]

The title compound was prepared in a procedure similar to that in Example 447. [M+H]⁺=1008.7.

### Example 647: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-fluoro-3,3-dimethyl-2-oxoindolin-1-yl)piperidine-2,6-dione

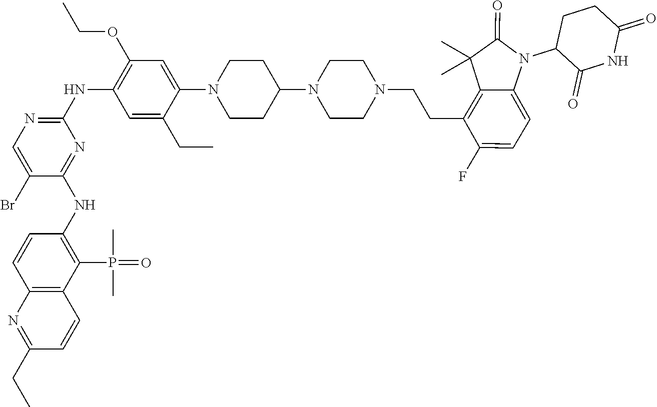 [SMILES: CCOc1cc(N2CCC(N3CCN(CCc4c(F)ccc5c4C(C)(C)C(=O)N5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 413. [M+H]⁺=1052.1.

### Example 648: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-5-fluoro-3-methyl-2-oxo-2,3-dihydro-1H-benzo[d]imidazol-1-yl)piperidine-2,6-dione

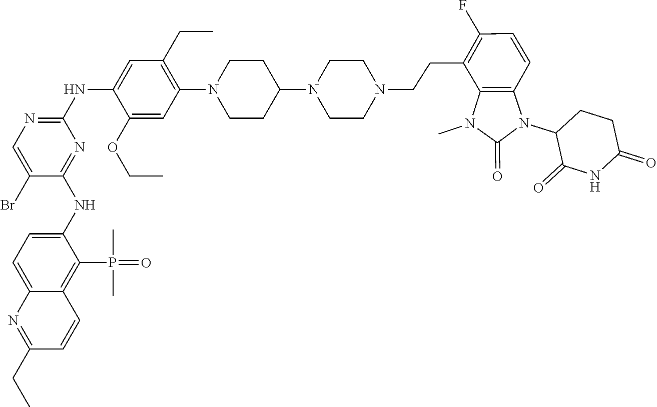 [SMILES: CCOc1cc(N2CCC(N3CCN(CCc4c(F)ccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 318. [M+H]⁺=1038.4.

### Example 649: (R)-3-(4-((R)-3-(4-(1-(4-((5-bromo-4-((5-(dimethylphosphoryl)-2-ethylquinolin-6-yl)amino)pyrimidin-2-yl)amino)-2-ethyl-5-(trifluoromethoxy)phenyl)piperidin-4-yl)piperazine-1-carbonyl)pyrrolidin-1-yl)-2,6-difluorophenyl)piperidine-2,6-dione

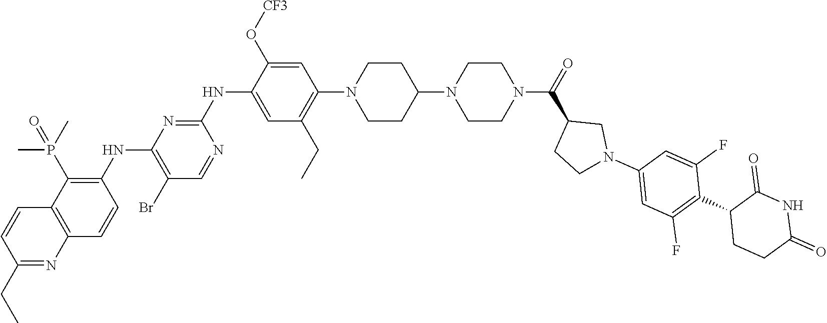 [SMILES: CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC(F)(F)F)ncc3Br)ccc2n1]

The title compound was prepared in a procedure similar to that in Example 488. [M+H]⁺=1095.8.

### Example 650: 3-(4-((S)-3-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)pyrrolidin-1-yl)-2,6-difluorophenyl)piperidine-2,6-dione

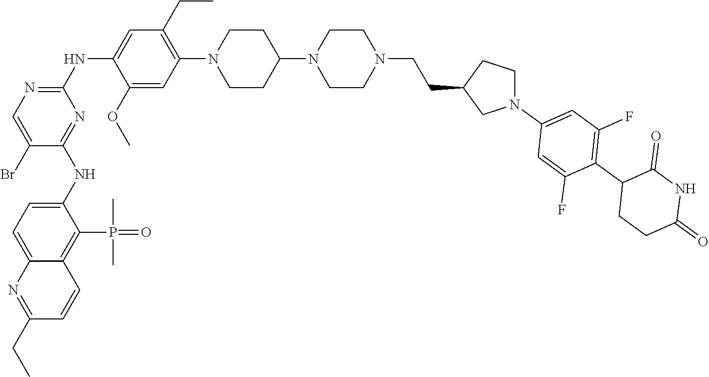 [SMILES: CCc1ccc2c(P(C)(C)=O)c(Nc3nc(Nc4cc(CC)c(N5CCC(N6CCN(CC[C@H]7CCN(c8cc(F)c(C9CCC(=O)NC9=O)c(F)c8)C7)CC6)CC5)cc4OC)ncc3Br)ccc2n1]

The title compound was prepared in a procedure similar to that in Example 484. [M+H]⁺=1041.4.

### Example 651: 3-(6-(2-(4-(1-(4-((5-bromo-4-((5-(dimethylphosphoryl)-3-fluoro-2-methylquinolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-dione

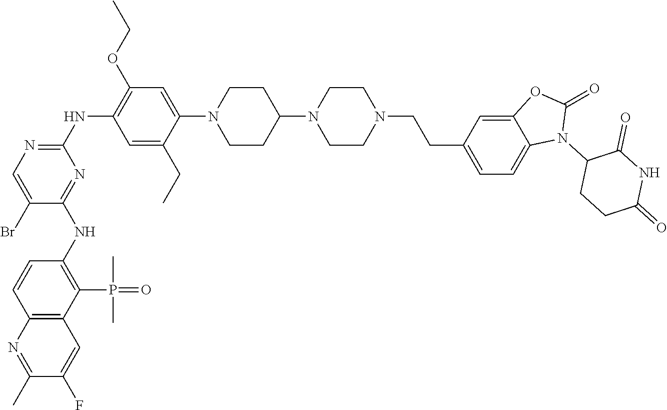 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2ccc3nc(C)c(F)cc3c2P(C)(C)=O)n1]

The title compound was prepared in a procedure similar to that in Example 492. [M+H]⁺=1011.7.

### Example 652: (R)-3-(4-((R)-3-(4-(1-(4-((5-bromo-4-((2-(cyclopropylamino)-5-(dimethylphosphoryl)quinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

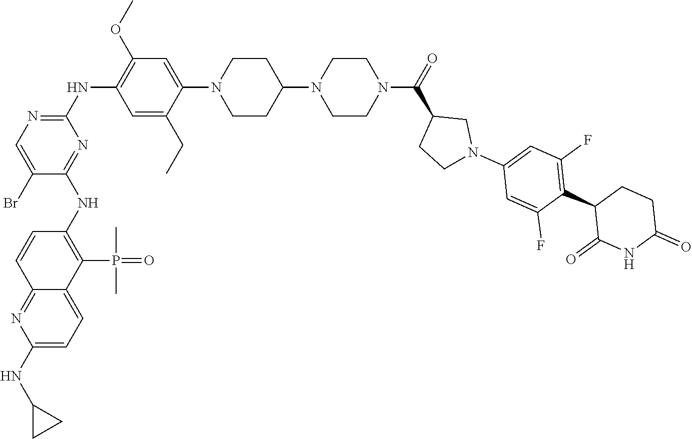 [SMILES: CCc1cc(Nc2ncc(Br)c(Nc3ccc4nc(NC5CC5)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound was prepared in a procedure similar to that in Example 488. [M+H]⁺=1068.4.

### Example 653: (R)-3-(4-(2-(4-(1-(4-((5-bromo-4-((2-(cyclopropylamino)-5-(dimethylphosphoryl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

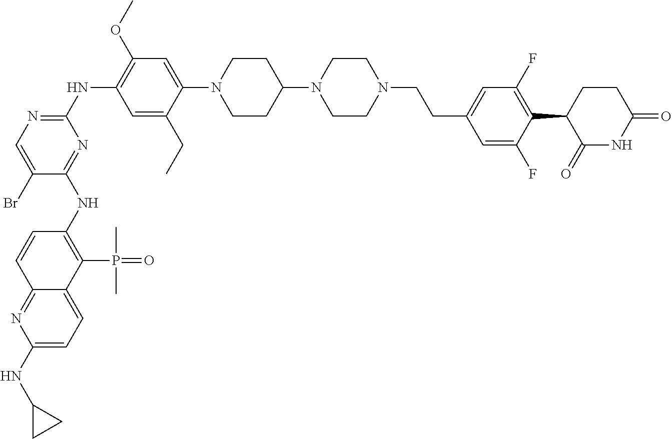 [SMILES: CCc1cc(Nc2ncc(Br)c(Nc3ccc4nc(NC5CC5)ccc4c3P(C)(C)=O)n2)c(OC)cc1N1CCC(N2CCN(CCc3cc(F)c([C@H]4CCC(=O)NC4=O)c(F)c3)CC2)CC1]

The title compound was prepared in a procedure similar to that in Example 484. [M+H]⁺=998.35.

### Example 654: (R)-3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)phenyl)piperidine-2,6-dione

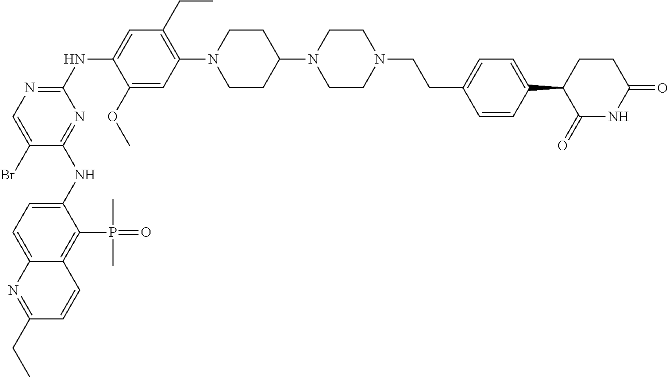 [SMILES: CCc1ccc2c(P(C)(C)=O)c(Nc3nc(Nc4cc(CC)c(N5CCC(N6CCN(CCc7ccc([C@H]8CCC(=O)NC8=O)cc7)CC6)CC5)cc4OC)ncc3Br)ccc2n1]

The title compound was prepared in a procedure similar to that in Example 492. [M+H]⁺=936.4.

### Example 655: (R)-3-(4-((R)-3-(4-(1-(4-((5-bromo-4-((5-(dimethylphosphoryl)-2-ethylquinolin-6-yl)amino)pyrimidin-2-yl)amino)-5-cyclopropoxy-2-ethylphenyl)piperidin-4-yl)piperazine-1-carbonyl)pyrrolidin-1-yl)-2,6-difluorophenyl)piperidine-2,6-dione

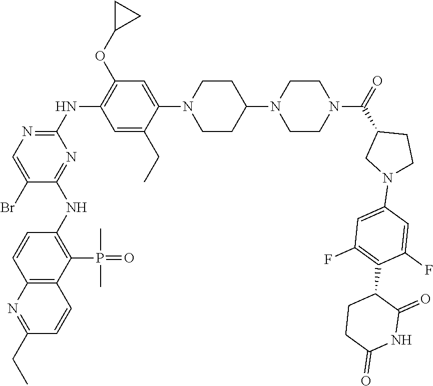 [SMILES: CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC4CC4)ncc3Br)ccc2n1]

The title compound was prepared in a procedure similar to that in Example 488. [M+H]⁺=1067.8.

### Example 656: (R)-3-(4-((R)-3-(4-(1-(4-((5-bromo-4-((8-(dimethylphosphoryl)cinnolin-7-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

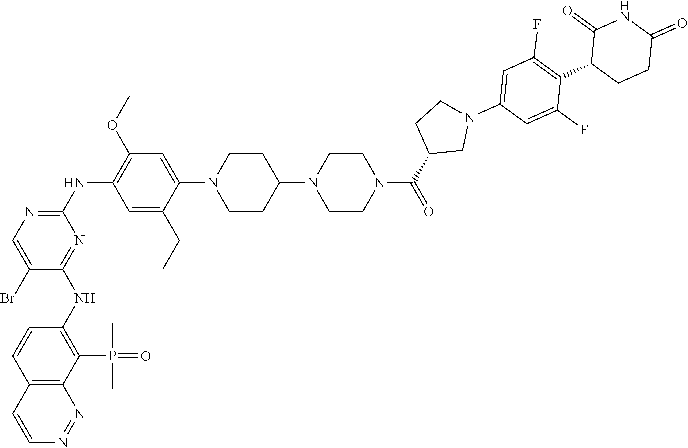 [SMILES: CCc1cc(Nc2ncc(Br)c(Nc3ccc4ccnnc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound was prepared in a procedure similar to that in Example 488. [M+H]⁺=1014.7.

### Example 657: (R)-3-(4-(2-((S)-4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)-2-(hydroxymethyl)piperazin-1-yl)ethyl)-2,6-difluorophenyl)piperidine-2,6-dione

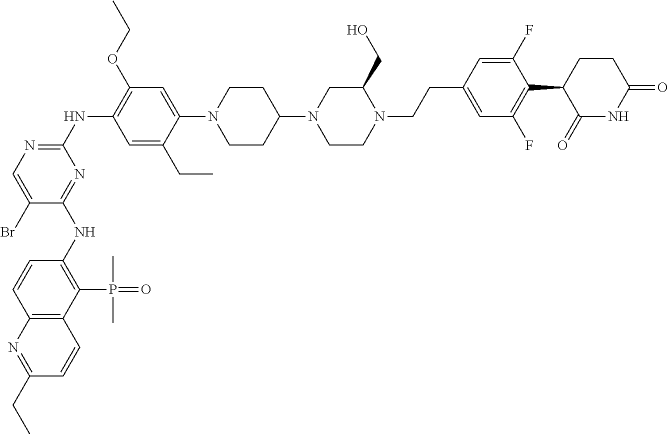 [SMILES: CCOc1cc(N2CCC(N3CCN(CCc4cc(F)c([C@H]5CCC(=O)NC5=O)c(F)c4)[C@H](CO)C3)CC2)c(CC)cc1Nc1ncc(Br)c(Nc2ccc3nc(CC)ccc3c2P(C)(C)=O)n1]

The title compound was prepared in a procedure similar to that in Example 484. [M+H]⁺=1017.2.

### Example 658: 3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)-2-oxoethyl)-3,3,5-trimethyl-2-oxoindolin-1-yl)piperidine-2,6-dione

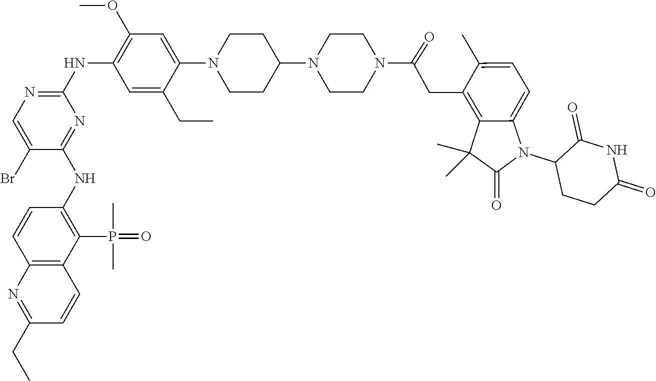 [SMILES: CCc1ccc2c(P(C)(C)=O)c(Nc3nc(Nc4cc(CC)c(N5CCC(N6CCN(C(=O)Cc7c(C)ccc8c7C(C)(C)C(=O)N8C7CCC(=O)NC7=O)CC6)CC5)cc4OC)ncc3Br)ccc2n1]

The title compound was prepared in a procedure similar to that in Example 447. [M+H]⁺=1047.1.

### Example 659: 3-(5′-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-7′-fluoro-2′-oxospiro[cyclopropane-1,3′-indolin]-1′-yl)piperidine-2,6-dione

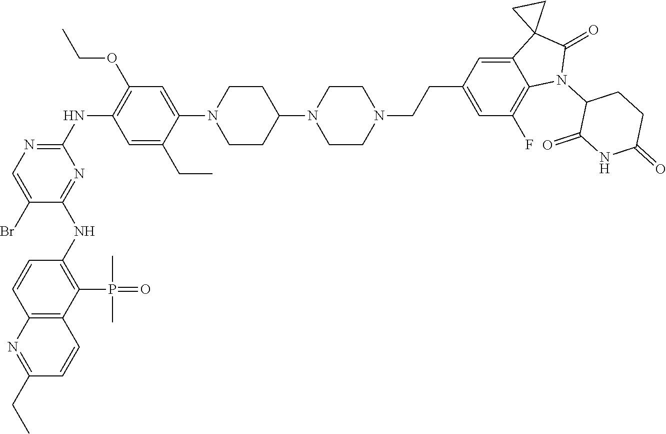 [SMILES: CCOc1cc(N2CCC(N3CCN(CCc4cc(F)c5c(c4)C4(CC4)C(=O)N5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 413. [M+H]⁺=1049.8.

### Example 660: 3-(4-((R)-3-(4-(1-(4-((5-bromo-4-((5-(dimethylphosphoryl)-3-methylisoquinolin-6-yl)amino)pyrimidin-2-yl)amino)-5-ethoxy-2-ethylphenyl)piperidin-4-yl)piperazine-1-carbonyl)pyrrolidin-1-yl)-2,6-difluorophenyl)piperidine-2,6-dione

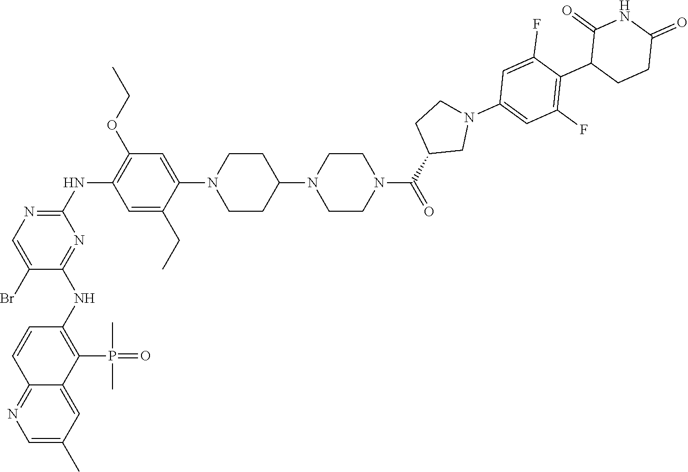 [SMILES: CCOc1cc(N2CCC(N3CCN(C(=O)[C@@H]4CCN(c5cc(F)c(C6CCC(=O)NC6=O)c(F)c5)C4)CC3)CC2)c(CC)cc1Nc1ncc(Br)c(Nc2ccc3ncc(C)cc3c2P(C)(C)=O)n1]

The title compound was prepared in a procedure similar to that in Example 488. [M+H]⁺=1041.4.

### Example 661: (R)-3-(4-(2-(4-(1-(4-((5-bromo-4-((7-(dimethylphosphoryl)-2-methylbenzo[d]thiazol-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

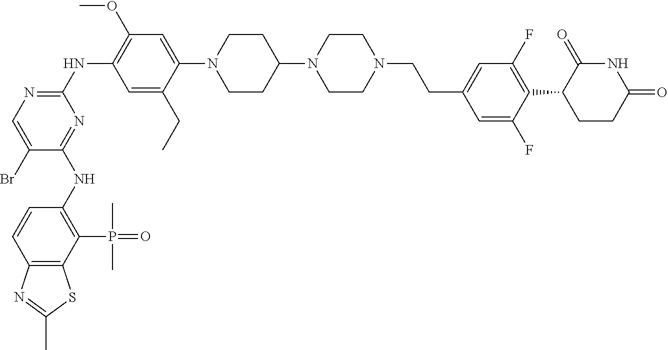 [SMILES: CCc1cc(Nc2ncc(Br)c(Nc3ccc4nc(C)sc4c3P(C)(C)=O)n2)c(OC)cc1N1CCC(N2CCN(CCc3cc(F)c([C@H]4CCC(=O)NC4=O)c(F)c3)CC2)CC1]

The title compound was prepared in a procedure similar to that in Example 484. [M+H]⁺=965.2.

### Example 662: (R)-3-(4-((S)-4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

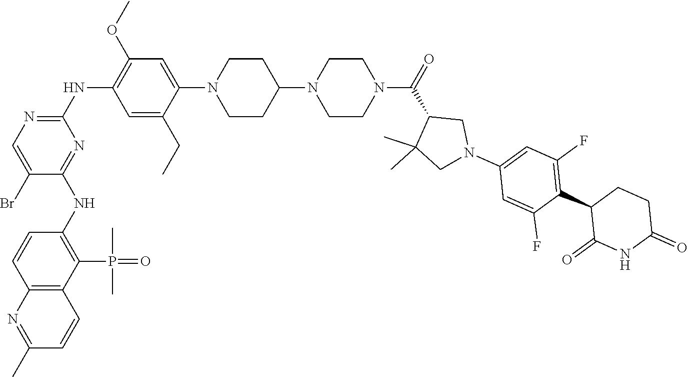 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CN(c4cc(F)c([C@H]5CCC(=O)NC5=O)c(F)c4)CC3(C)C)CC2)CC1]

The title compound was prepared in a procedure similar to that in Example 488. [M+H]⁺=1055.2.

### Example 663: (R)-3-(4-((4-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)bicyclo[2.2.1]heptan-1-yl)amino)-2,6-difluorophenyl)piperidine-2,6-dione

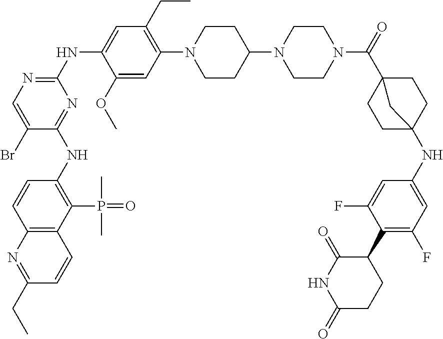 [SMILES: CCc1ccc2c(P(C)(C)=O)c(Nc3nc(Nc4cc(CC)c(N5CCC(N6CCN(C(=O)C78CCC(Nc9cc(F)c([C@H]%10CCC(=O)NC%10=O)c(F)c9)(CC7)C8)CC6)CC5)cc4OC)ncc3Br)ccc2n1]

The title compound was prepared in a procedure similar to that in Example 488. [M+H]⁺=1081.4.

### Example 664: (R)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione

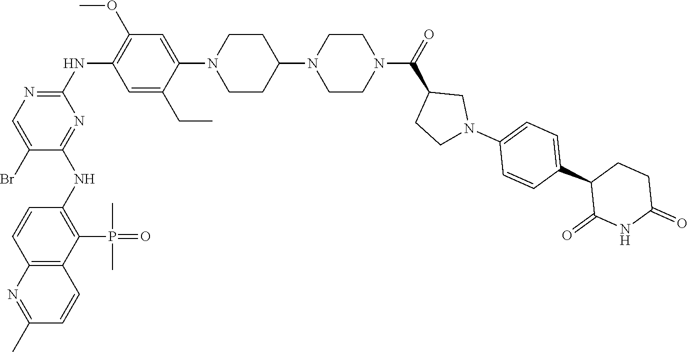 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4ccc([C@H]5CCC(=O)NC5=O)cc4)C3)CC2)CC1]

The title compound was prepared in a procedure similar to that in Example 488. [M+H]⁺=992.2.

### Example 665: (R)-3-(4-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)-2-oxoethyl)piperazin-1-yl)-2,6-difluorophenyl)piperidine-2,6-dione

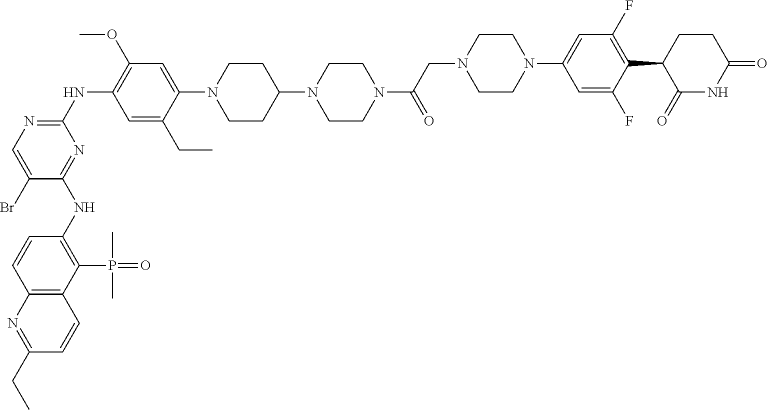 [SMILES: CCc1ccc2c(P(C)(C)=O)c(Nc3nc(Nc4cc(CC)c(N5CCC(N6CCN(C(=O)CN7CCN(c8cc(F)c([C@H]9CCC(=O)NC9=O)c(F)c8)CC7)CC6)CC5)cc4OC)ncc3Br)ccc2n1]

The title compound was prepared in a procedure similar to that in Example 488. [M+H]⁺=1071.1.

### Example 666: 3-(4-(2-(4-(1-(4-((5-bromo-4-((7-(dimethylphosphoryl)-2-methylbenzo[d]thiazol-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

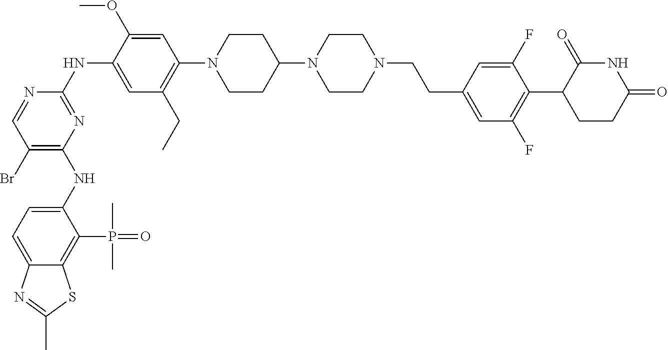 [SMILES: CCc1cc(Nc2ncc(Br)c(Nc3ccc4nc(C)sc4c3P(C)(C)=O)n2)c(OC)cc1N1CCC(N2CCN(CCc3cc(F)c(C4CCC(=O)NC4=O)c(F)c3)CC2)CC1]

The title compound was prepared in a procedure similar to that in Example 484. [M+H]⁺=964.6.

### Example 667: (R)-3-(4-((R)-3-(9-(4-((5-bromo-4-((5-(dimethylphosphoryl)quinoxalin-6-yl)amino)pyrimidin-2-yl)amino)-5-methoxy-2-methylphenyl)-3,9-diazaspiro[5.5]undecane-3-carbonyl)pyrrolidin-1-yl)-2,6-difluorophenyl)piperidine-2,6-dione

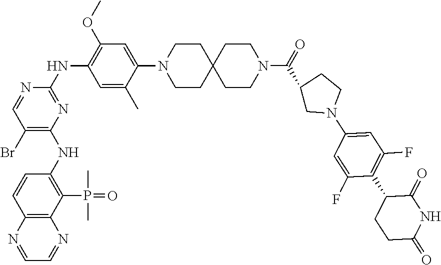 [SMILES: COc1cc(N2CCC3(CCN(C(=O)[C@@H]4CCN(c5cc(F)c([C@H]6CCC(=O)NC6=O)c(F)c5)C4)CC3)CC2)c(C)cc1Nc1ncc(Br)c(Nc2ccc3nccnc3c2P(C)(C)=O)n1]

The title compound was prepared in a procedure similar to that in Example 488. [M+H]⁺=985.4.

### Example 668: (R)-3-(4-((R)-3-(4-(1-(4-((5-bromo-4-((5-(dimethylphosphoryl)quinazolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

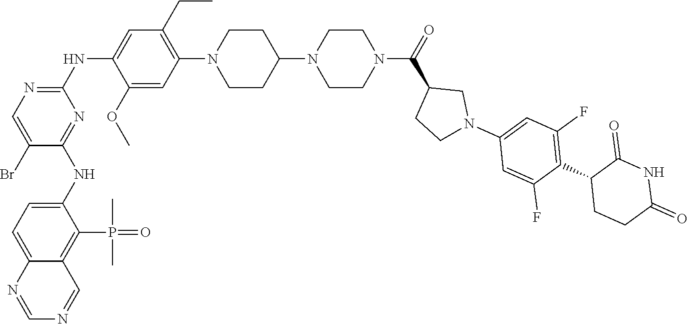 [SMILES: CCc1cc(Nc2ncc(Br)c(Nc3ccc4ncncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound was prepared in a procedure similar to that in Example 488. [M+H]⁺=1014.3.

### Example 669: 3-(6-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)-2-oxoethyl)-2-oxobenzo[d]oxazol-3(2H)-yl)piperidine-2,6-dione

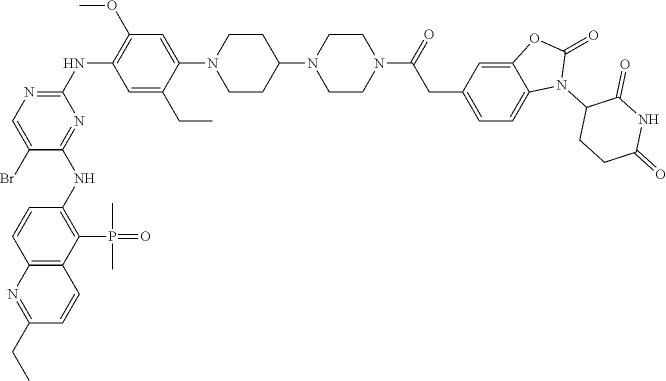 [SMILES: CCc1ccc2c(P(C)(C)=O)c(Nc3nc(Nc4cc(CC)c(N5CCC(N6CCN(C(=O)Cc7ccc8c(c7)oc(=O)n8C7CCC(=O)NC7=O)CC6)CC5)cc4OC)ncc3Br)ccc2n1]

The title compound was prepared in a procedure similar to that in Example 447. [M+H]⁺=1008.5.

### Example 670: (R)-3-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)-2-oxoethyl)-2,6-difluorophenyl)piperidine-2,6-dione

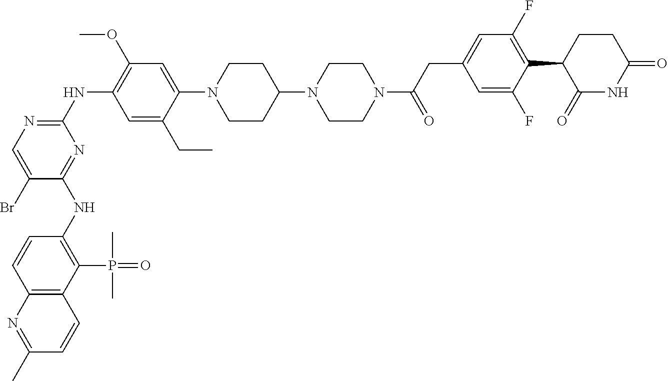 [SMILES: CCc1cc(Nc2ncc(Br)c(Nc3ccc4nc(C)ccc4c3P(C)(C)=O)n2)c(OC)cc1N1CCC(N2CCN(C(=O)Cc3cc(F)c([C@H]4CCC(=O)NC4=O)c(F)c3)CC2)CC1]

The title compound was prepared in a procedure similar to that in Example 488. [M+H]⁺=973.2.

### Example 671: 3-(4-(2-(4-(1-(4-((5-bromo-4-((5-(dimethylphosphoryl)-2-ethyl-8-fluoroquinolin-6-yl)amino)pyrimidin-2-yl)amino)-2-ethyl-5-methoxyphenyl)piperidin-4-yl)piperazin-1-yl)-2-oxoethyl)-3-methyl-2-oxo-2,3-dihydro-1H-benzo[d]imidazol-1-yl)piperidine-2,6-dione

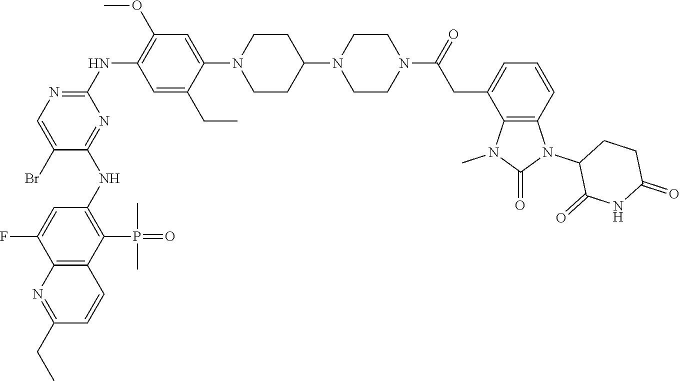 [SMILES: CCc1ccc2c(P(C)(C)=O)c(Nc3nc(Nc4cc(CC)c(N5CCC(N6CCN(C(=O)Cc7cccc8c7n(C)c(=O)n8C7CCC(=O)NC7=O)CC6)CC5)cc4OC)ncc3Br)cc(F)c2n1]

The title compound was prepared in a procedure similar to that in Example 447. [M+H]⁺=1038.3.

### Example 672: (R)-3-(4-(2-(4-(1-(4-((5-bromo-4-((5-(dimethylphosphoryl)-8-fluoro-3-methylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

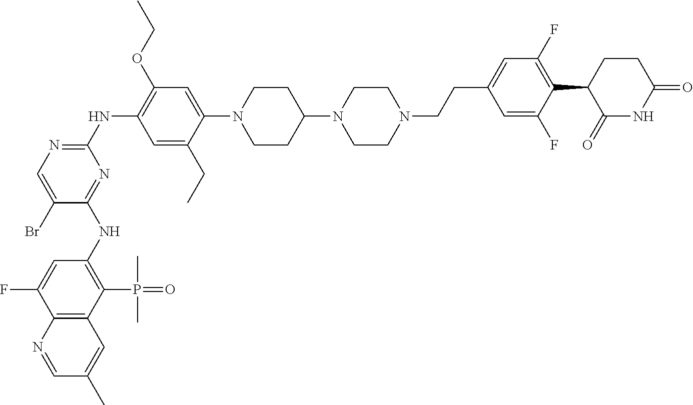 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2cc(F)c3ncc(C)cc3c2P(C)(C)=O)n1]

The title compound was prepared in a procedure similar to that in Example 484. [M+H]⁺=990.2.

### Example 673: (R)-3-(4-((R)-3-(4-(1-(4-((5-bromo-4-((5-(dimethylphosphoryl)-2-ethyl-1-oxo-1,2-dihydrophthalazin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

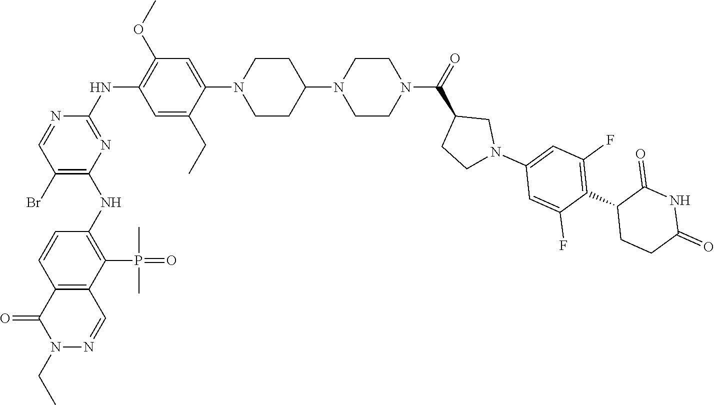 [SMILES: CCc1cc(Nc2ncc(Br)c(Nc3ccc4c(=O)n(CC)ncc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound was prepared in a procedure similar to that in Example 488. [M+H]⁺=1058.4.

### Example 674: 6-((5-bromo-2-((4-(4-(4-((R)-1-(4-((R)-2,6-dioxopiperidin-3-yl)-3,5-difluorophenyl)pyrrolidine-3-carbonyl)piperazin-1-yl)piperidin-1-yl)-5-ethyl-2-methoxyphenyl)amino)pyrimidin-4-yl)amino)-5-(dimethylphosphoryl)quinoline-2-carbonitrile

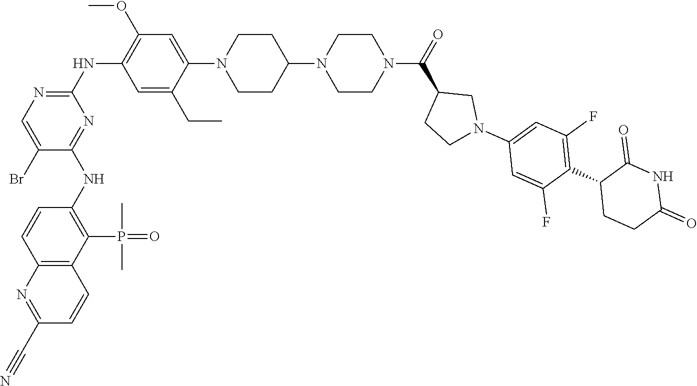 [SMILES: CCc1cc(Nc2ncc(Br)c(Nc3ccc4nc(C#N)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound was prepared in a procedure similar to that in Example 488. [M+H]⁺=1038.1.

### Example 675: (R)-3-(4-((R)-3-((S)-4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)-2-(hydroxymethyl)piperazine-1-carbonyl)pyrrolidin-1-yl)-2,6-difluorophenyl)piperidine-2,6-dione

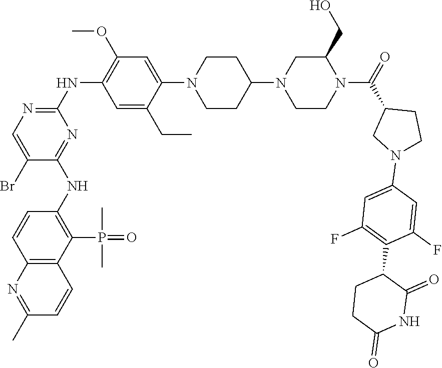 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)[C@H](CO)C2)CC1]

The title compound was prepared in a procedure similar to that in Example 488. [M+H]⁺=1057.2.

### Example 676: 3-(6-(2-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazin-1-yl)ethyl)-2-oxobenzo[d]oxazol-3(2H)-yl)piperidine-2,6-diQne

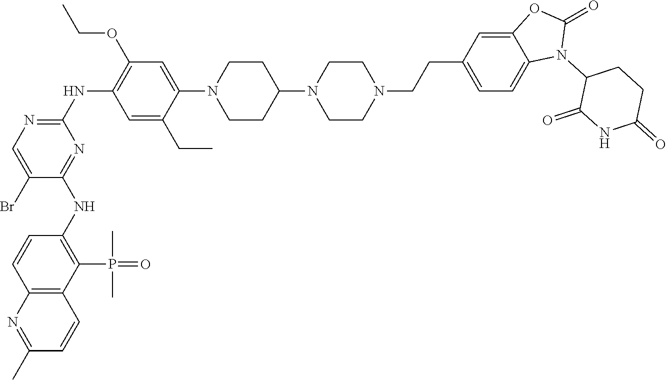 [SMILES: CCOc1cc(N2CCC(N3CCN(CCc4ccc5c(c4)oc(=O)n5C4CCC(=O)NC4=O)CC3)CC2)c(CC)cc1Nc1ncc(Br)c(Nc2ccc3nc(C)ccc3c2P(C)(C)=O)n1]

The title compound was prepared in a procedure similar to that in Example 492. [M+H]⁺=993.2.

### Example 677: (R)-3-(4-((R)-3-(4-(1-(4-((5-bromo-4-((5-(dimethylphosphoryl)-2-ethoxyquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

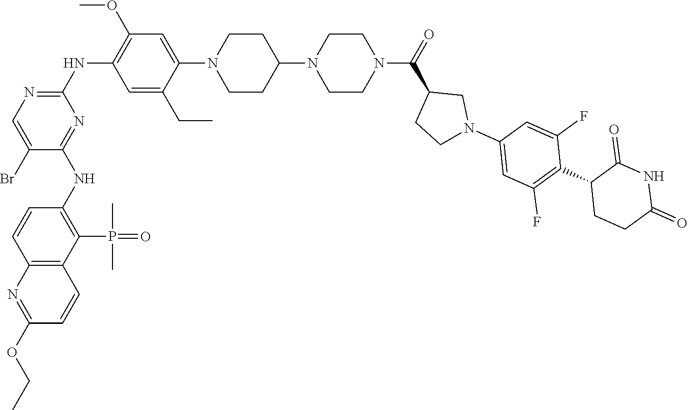 [SMILES: CCOc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1]

The title compound was prepared in a procedure similar to that in Example 488. [M+H]⁺=1057.5.

### Example 678: (R)-3-(4-((R)-3-(4-(1-(4-((5-bromo-4-((5-(dimethylphosphoryl)-8-fluoroquinolin-6-yl)amino)pyrimidin-2-yl)amino)-5-ethoxy-2-ethylphenyl)piperidin-4-yl)piperazine-1-carbonyl)pyrrolidin-1-yl)-2,6-difluorophenyl)piperidine-2,6-dione

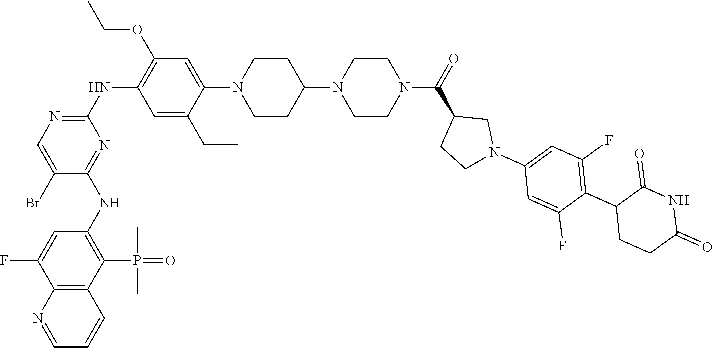 [SMILES: CCOc1cc(N2CCC(N3CCN(C(=O)[C@@H]4CCN(c5cc(F)c(C6CCC(=O)NC6=O)c(F)c5)C4)CC3)CC2)c(CC)cc1Nc1ncc(Br)c(Nc2cc(F)c3ncccc3c2P(C)(C)=O)n1]

The title compound was prepared in a procedure similar to that in Example 488. [M+H]⁺=1045.2.

### Example 679: (R)-3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

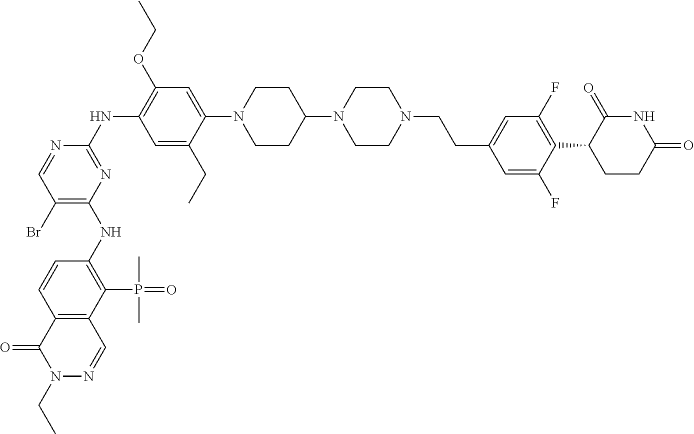 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3c(=O)n(CC)ncc3c2P(C)(C)=O)n1]

The title compound was prepared in a procedure similar to that in Example 484. [M+H]⁺=1003.7.

### Example 680: 3-(4-((R)-3-(4-(1-(4-((5-bromo-4-((5-(dimethylphosphoryl)-2-(2-hydroxypropan-2-yl)quinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

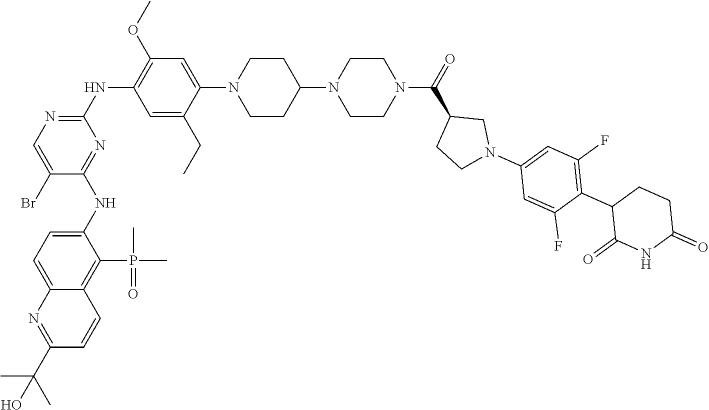 [SMILES: CCc1cc(Nc2ncc(Br)c(Nc3ccc4nc(C(C)(C)O)ccc4c3P(C)(C)=O)n2)c(OC)cc1N1CCC(N2CCN(C(=O)[C@@H]3CCN(c4cc(F)c(C5CCC(=O)NC5=O)c(F)c4)C3)CC2)CC1]

The title compound was prepared in a procedure similar to that in Example 488. [M+H]⁺=1071.4.

### Example 681: 3-(4-(((1s,4s)-4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)cyclohexyl)oxy)-2,6-difluorophenyl)piperidine-2,6-dione

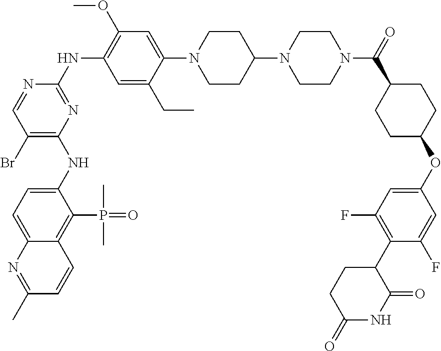 [SMILES: CCc1cc(Nc2ncc(Br)c(Nc3ccc4nc(C)ccc4c3P(C)(C)=O)n2)c(OC)cc1N1CCC(N2CCN(C(=O)[C@H]3CC[C@@H](Oc4cc(F)c(C5CCC(=O)NC5=O)c(F)c4)CC3)CC2)CC1]

The title compound was prepared in a procedure similar to that in Example 488. [M+H]⁺=1056.7.

### Example 682: (R)-6-((5-bromo-2-((4-(4-(4-(4-(2,6-dioxopiperidin-3-yl)-3,5-difluorophenethyl)piperazin-1-yl)piperidin-1-yl)-5-ethyl-2-methoxyphenyl)amino)pyrimidin-4-yl)amino)-5-(dimethylphosphoryl)quinoline-2-carbonitrile

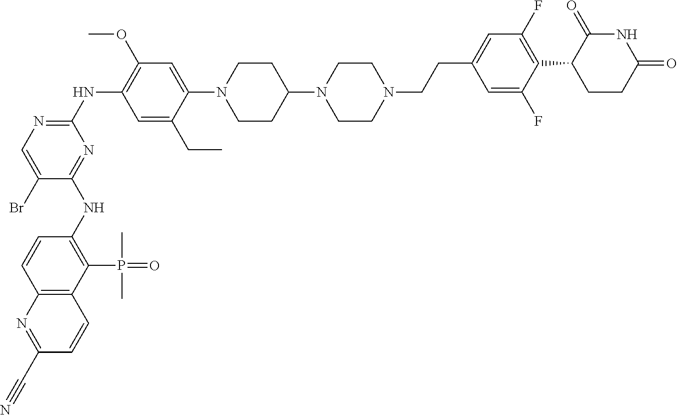 [SMILES: CCc1cc(Nc2ncc(Br)c(Nc3ccc4nc(C#N)ccc4c3P(C)(C)=O)n2)c(OC)cc1N1CCC(N2CCN(CCc3cc(F)c([C@H]4CCC(=O)NC4=O)c(F)c3)CC2)CC1]

The title compound was prepared in a procedure similar to that in Example 484. [M+H]⁺=969.9.

### Example 683: 3-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethoxy)-3-methyl-2-oxo-2,3-dihydro-1H-benzo[d]imidazol-1-yl)piperidine-2,6-dione

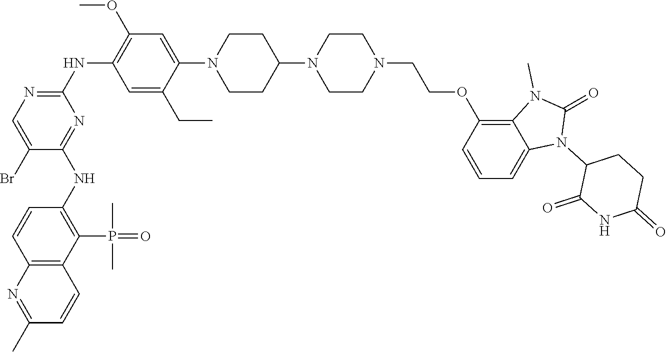 [SMILES: CCc1cc(Nc2ncc(Br)c(Nc3ccc4nc(C)ccc4c3P(C)(C)=O)n2)c(OC)cc1N1CCC(N2CCN(CCOc3cccc4c3n(C)c(=O)n4C3CCC(=O)NC3=O)CC2)CC1]

The title compound was prepared in a procedure similar to that in Example 318. [M+H]⁺=1008.8.

### Example 684: (R)-3-(4-(2-(4-(1-(4-((5-bromo-4-((5-(dimethylphosphoryl)-1-methylisoquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

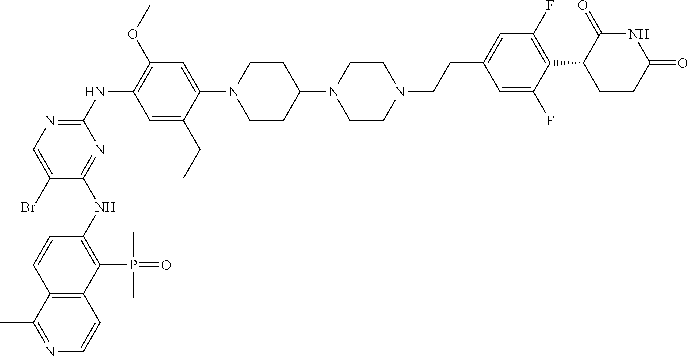 [SMILES: CCc1cc(Nc2ncc(Br)c(Nc3ccc4c(C)nccc4c3P(C)(C)=O)n2)c(OC)cc1N1CCC(N2CCN(CCc3cc(F)c([C@H]4CCC(=O)NC4=O)c(F)c3)CC2)CC1]

The title compound was prepared in a procedure similar to that in Example 484. [M+H]⁺=958.7.

### Example 685: (R)-3-(4-((R)-3-(4-(1-(4-((5-bromo-4-((7-(dimethylphosphoryl)-2-methylbenzo[d]thiazol-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

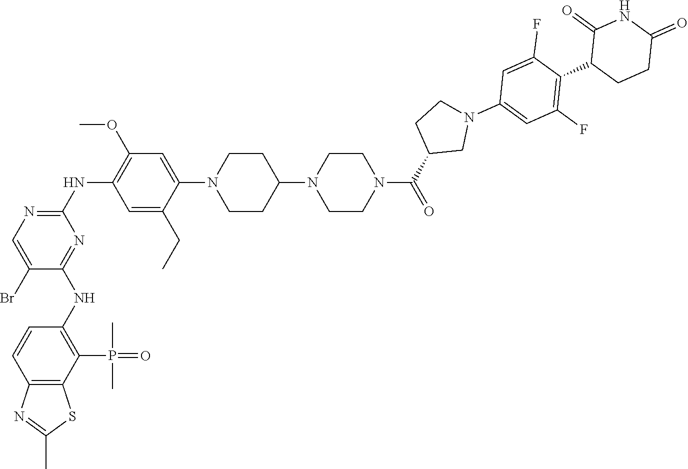

The title compound was prepared in a procedure similar to that in Example 488. [M+H]⁺=1033.7.

### Example 686: (R)-3-(4-((R)-3-(4-(1-(4-((5-bromo-4-((5-(dimethylphosphoryl)-2,3-dihydrobenzo[b][1,4]dioxin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

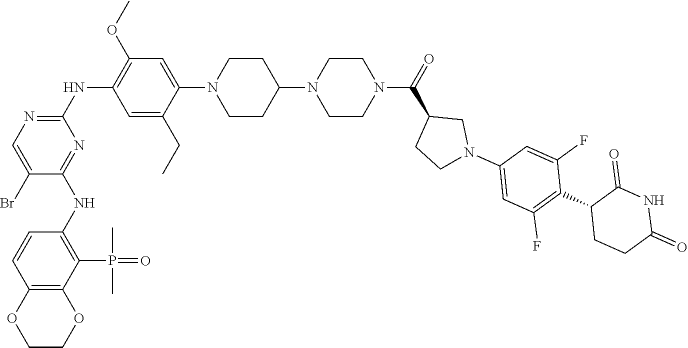 [SMILES: CCc1cc(Nc2ncc(Br)c(Nc3ccc4c(c3P(C)(C)=O)OCCO4)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound was prepared in a procedure similar to that in Example 488. [M+H]⁺=1019.6.

### Example 687: 3-(4-(1-(1-(1-(4-((5-bromo-4-((5-(dimethylphosphoryl)-2-methylquinolin-6-yl)amino)pyrimidin-2-yl)amino)-5-methoxy-2-methylphenyl)piperidin-4-yl)azetidine-3-carbonyl)piperidin-4-yl)-2,6-difluorophenyl)piperidine-2,6-dione

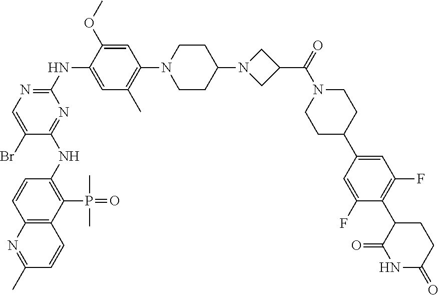 [SMILES: COc1cc(N2CCC(N3CC(C(=O)N4CCC(c5cc(F)c(C6CCC(=O)NC6=O)c(F)c5)CC4)C3)CC2)c(C)cc1Nc1ncc(Br)c(Nc2ccc3nc(C)ccc3c2P(C)(C)=O)n1]

The title compound was prepared in a procedure similar to that in Example 488. [M+H]⁺=998.7.

### Example 689: 3-(4-(2-(4-(1-(4-((4-((5-(dimethylphosphoryl)-2-methylquinolin-6-yl)amino)-5-(trifluoromethyl)pyrimidin-2-yl)amino)-2-ethyl-5-methoxyphenyl)piperidin-4-yl)piperazin-1-yl)ethyl)-2,6-difluorophenyl)piperidine-2,6-dione

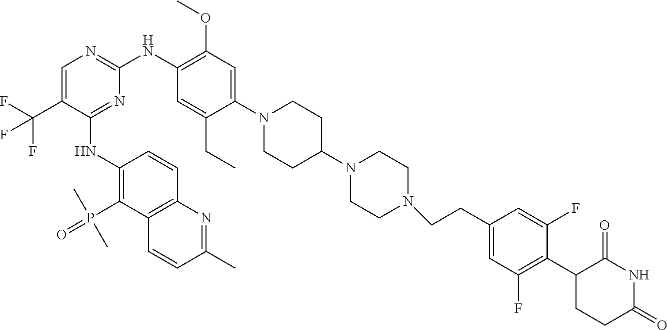 [SMILES: CCc1cc(Nc2ncc(C(F)(F)F)c(Nc3ccc4nc(C)ccc4c3P(C)(C)=O)n2)c(OC)cc1N1CCC(N2CCN(CCc3cc(F)c(C4CCC(=O)NC4=O)c(F)c3)CC2)CC1]

The title compound was prepared in a procedure similar to that in Example 484. [M+H]⁺=948.5.

### Example 690: 3-(4-(2-(4-(1-(4-((5-bromo-4-((5-(dimethylphosphoryl)-2-(methoxymethyl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

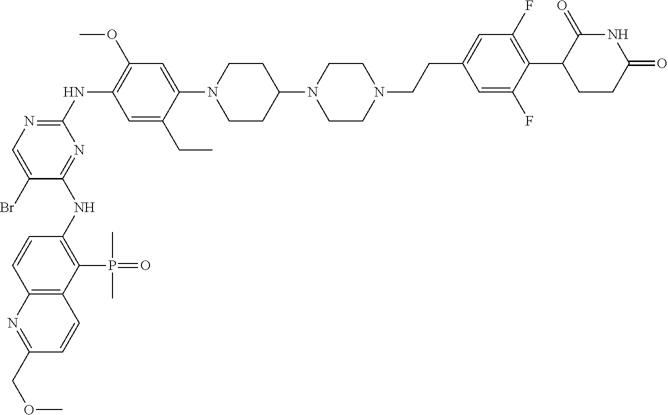 [SMILES: CCc1cc(Nc2ncc(Br)c(Nc3ccc4nc(COC)ccc4c3P(C)(C)=O)n2)c(OC)cc1N1CCC(N2CCN(CCc3cc(F)c(C4CCC(=O)NC4=O)c(F)c3)CC2)CC1]

The title compound was prepared in a procedure similar to that in Example 484. [M+H]⁺=988.6.

### Example 691: 3-(4-(((1r,3r)-3-((4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)methyl)cyclobutyl)(methyl)amino)-2,6-difluorophenyl)piperidine-2,6-dione

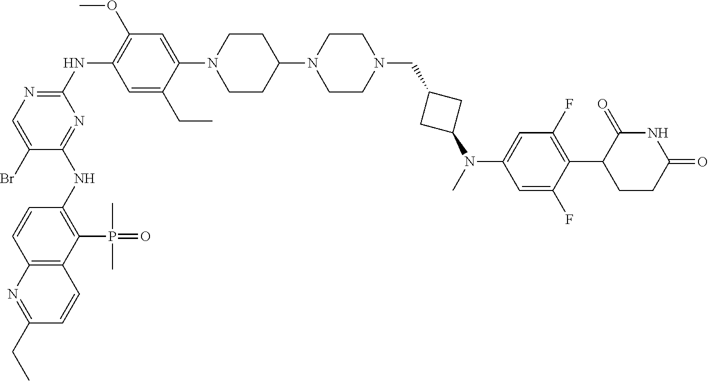 [SMILES: CCc1ccc2c(P(C)(C)=O)c(Nc3nc(Nc4cc(CC)c(N5CCC(N6CCN(C[C@H]7C[C@H](N(C)c8cc(F)c(C9CCC(=O)NC9=O)c(F)c8)C7)CC6)CC5)cc4OC)ncc3Br)ccc2n1]

The title compound was prepared in a procedure similar to that in Example 484. [M+H]⁺=1041.2.

### Example 692: 3-(4-(3-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)azetidin-1-yl)-1-oxoisoindolin-2-yl)piperidine-2,6-dione

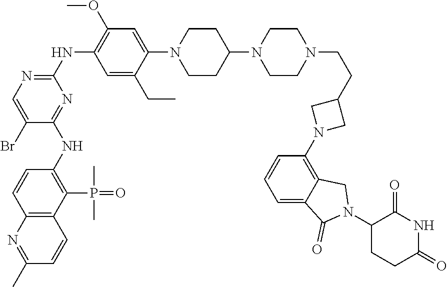 [SMILES: CCc1cc(Nc2ncc(Br)c(Nc3ccc4nc(C)ccc4c3P(C)(C)=O)n2)c(OC)cc1N1CCC(N2CCN(CCC3CN(c4cccc5c4CN(C4CCC(=O)NC4=O)C5=O)C3)CC2)CC1]

The title compound was prepared in a procedure similar to that in Example 492. [M+H]⁺=1032.1.

### Example 693: 3-(4-(2-((S)-4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)-2-methylpiperazin-1-yl)ethyl)-2,6-difluorophenyl)piperidine-2,6-dione

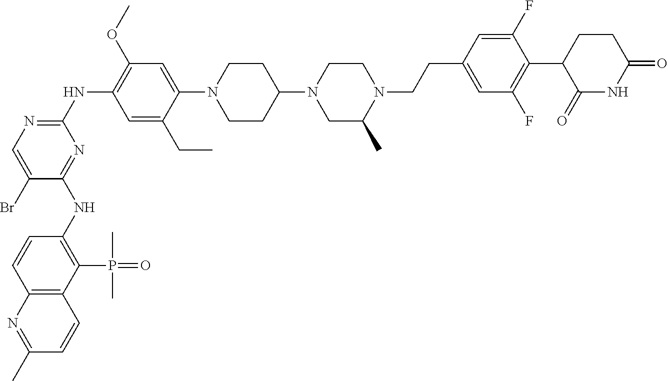 [SMILES: CCc1cc(Nc2ncc(Br)c(Nc3ccc4nc(C)ccc4c3P(C)(C)=O)n2)c(OC)cc1N1CCC(N2CCN(CCc3cc(F)c(C4CCC(=O)NC4=O)c(F)c3)[C@@H](C)C2)CC1]

The title compound was prepared in a procedure similar to that in Example 484. [M+H]⁺=972.1.

### Example 694: 3-(4-(4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)piperidin-1-yl)-2,6-difluorophenyl)piperidine-2,6-dione

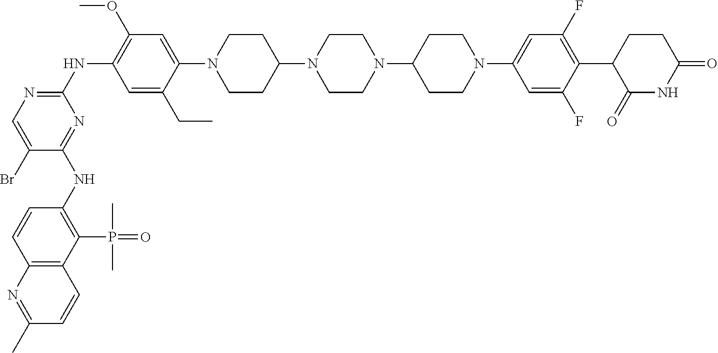 [SMILES: CCc1cc(Nc2ncc(Br)c(Nc3ccc4nc(C)ccc4c3P(C)(C)=O)n2)c(OC)cc1N1CCC(N2CCN(C3CCN(c4cc(F)c(C5CCC(=O)NC5=O)c(F)c4)CC3)CC2)CC1]

The title compound was prepared in a procedure similar to that in Example 484. [M+H]⁺=1013.9.

### Example 695: 3-(4-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)-2-oxopiperazin-1-yl)ethyl)piperidin-1-yl)-2,6-difluorophenyl)piperidine-2,6-dione

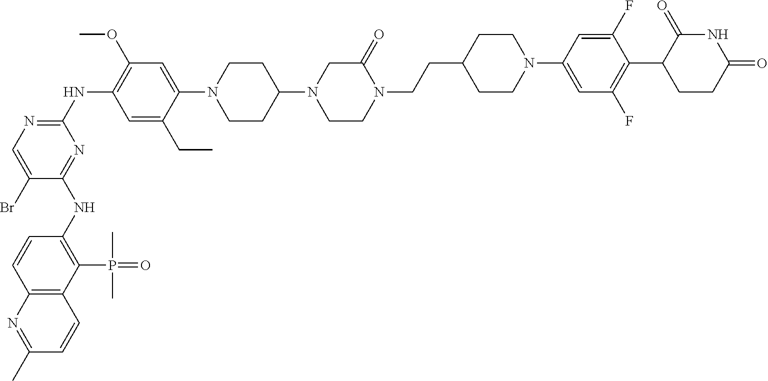 [SMILES: CCc1cc(Nc2ncc(Br)c(Nc3ccc4nc(C)ccc4c3P(C)(C)=O)n2)c(OC)cc1N1CCC(N2CCN(CCC3CCN(c4cc(F)c(C5CCC(=O)NC5=O)c(F)c4)CC3)C(=O)C2)CC1]

The title compound was prepared in a procedure similar to that in Example 484. [M+H]⁺=1055.4.

### Example 696: 3-(4-((R)-3-(4-(1-(4-((5-bromo-4-((5-(dimethylphosphoryl)-2-(hydroxymethyl)quinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

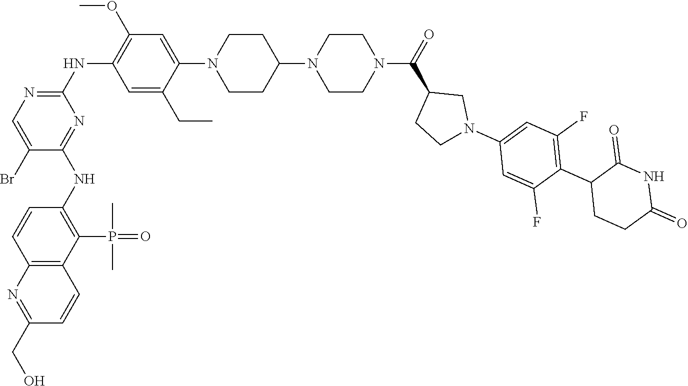 [SMILES: CCc1cc(Nc2ncc(Br)c(Nc3ccc4nc(CO)ccc4c3P(C)(C)=O)n2)c(OC)cc1N1CCC(N2CCN(C(=O)[C@@H]3CCN(c4cc(F)c(C5CCC(=O)NC5=O)c(F)c4)C3)CC2)CC1]

The title compound was prepared in a procedure similar to that in Example 488. [M+H]⁺=1043.3.

### Example 697: 3-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)-2-oxoethyl)-3-methyl-2-oxo-2,3-dihydro-1H-benzo[d]imidazol-1-yl)piperidine-2,6-dione

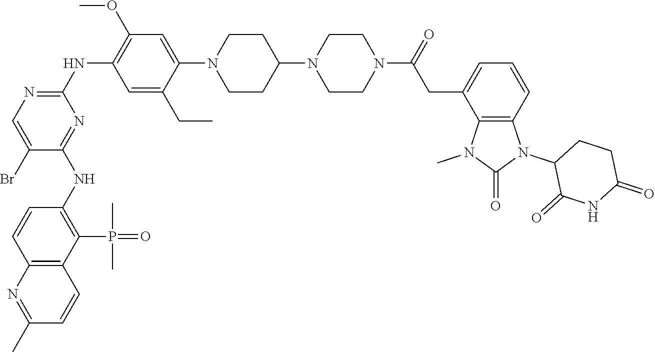 [SMILES: CCc1cc(Nc2ncc(Br)c(Nc3ccc4nc(C)ccc4c3P(C)(C)=O)n2)c(OC)cc1N1CCC(N2CCN(C(=O)Cc3cccc4c3n(C)c(=O)n4C3CCC(=O)NC3=O)CC2)CC1]

The title compound was prepared in a procedure similar to that in Example 447. [M+H]⁺=1006.3.

### Example 698: 3-(4-(2-(4-(1-(4-((5-bromo-4-((5-(dimethylphosphoryl)-3-methyl-4-oxo-3,4-dihydroquinazolin-6-yl)amino)pyrimidin-2-yl)amino)-2-ethyl-5-methoxyphenyl)piperidin-4-yl)piperazin-1-yl)ethyl)-3-methyl-2-oxo-2,3-dihydro-1H-benzo[d]imidazol-1-yl)piperidine-2,6-dione

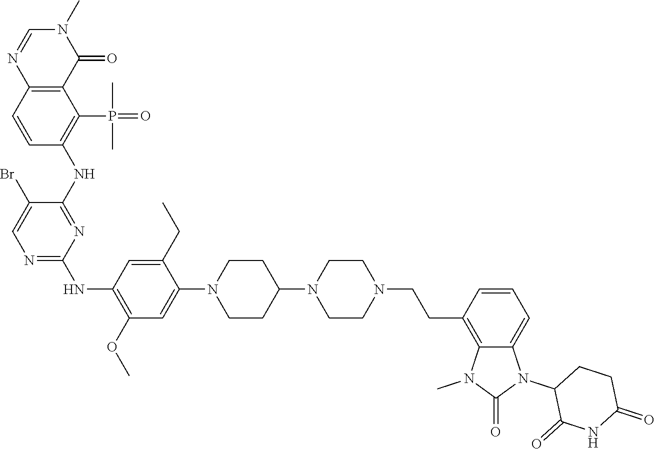 [SMILES: CCc1cc(Nc2ncc(Br)c(Nc3ccc4ncn(C)c(=O)c4c3P(C)(C)=O)n2)c(OC)cc1N1CCC(N2CCN(CCc3cccc4c3n(C)c(=O)n4C3CCC(=O)NC3=O)CC2)CC1]

The title compound was prepared in a procedure similar to that in Example 318. [M+H]⁺=1009.4.

### Example 699: 3-(4-((4-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)bicyclo[2.2.1]heptan-1-yl)amino)-2,6-difluorophenyl)piperidine-2,6-dione

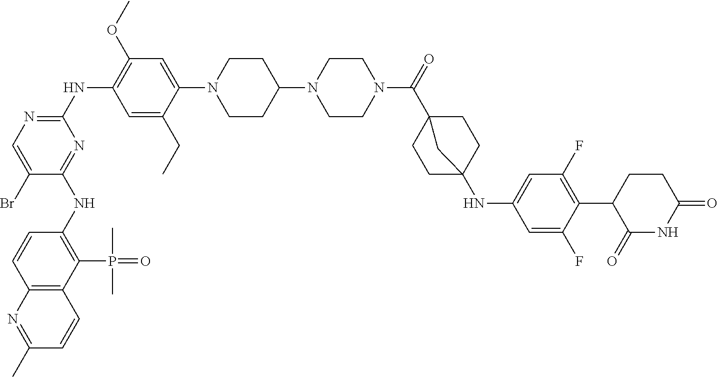 [SMILES: CCc1cc(Nc2ncc(Br)c(Nc3ccc4nc(C)ccc4c3P(C)(C)=O)n2)c(OC)cc1N1CCC(N2CCN(C(=O)C34CCC(Nc5cc(F)c(C6CCC(=O)NC6=O)c(F)c5)(CC3)C4)CC2)CC1]

The title compound was prepared in a procedure similar to that in Example 488. [M+H]⁺=1067.1.

### Example 700: 3-(4-(1-(1′-(4-((5-bromo-4-((5-(dimethylphosphoryl)-2-methylquinolin-6-yl)amino)pyrimidin-2-yl)amino)-2-ethyl-5-methoxyphenyl)-[1,4′-bipiperidine]-4-carbonyl)piperidin-4-yl)-2,6-difluorophenyl)piperidine-2,6-dione

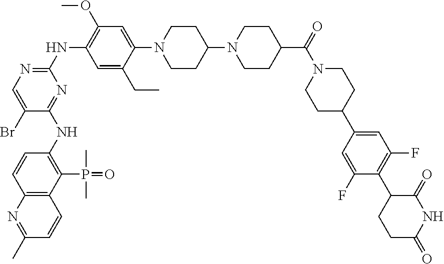 [SMILES: CCc1cc(Nc2ncc(Br)c(Nc3ccc4nc(C)ccc4c3P(C)(C)=O)n2)c(OC)cc1N1CCC(N2CCC(C(=O)N3CCC(c4cc(F)c(C5CCC(=O)NC5=O)c(F)c4)CC3)CC2)CC1]

The title compound was prepared in a procedure similar to that in Example 488. [M+H]⁺=1040.2.

### Example 701: 3-(4-(3-(4-(1-(4-((5-bromo-4-((5-(dimethylphosphoryl)-3-methyl-4-oxo-3,4-dihydroquinazolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

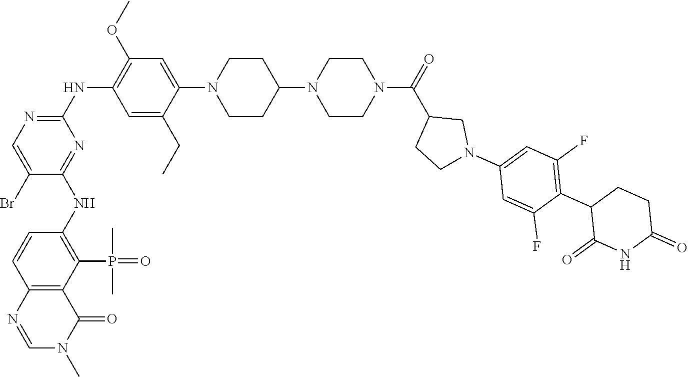 [SMILES: CCc1cc(Nc2ncc(Br)c(Nc3ccc4ncn(C)c(=O)c4c3P(C)(C)=O)n2)c(OC)cc1N1CCC(N2CCN(C(=O)C3CCN(c4cc(F)c(C5CCC(=O)NC5=O)c(F)c4)C3)CC2)CC1]

The title compound was prepared in a procedure similar to that in Example 488. [M+H]⁺=1044.3.

### Example 703: 3-(4-(4-(2-(4-(1-(4-((5-acetyl-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

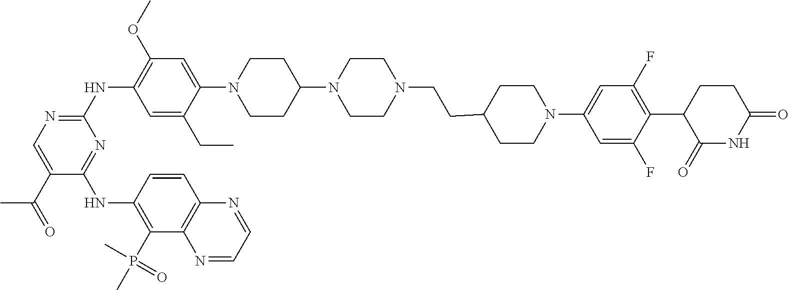 [SMILES: CCc1cc(Nc2ncc(C(C)=O)c(Nc3ccc4nccnc4c3P(C)(C)=O)n2)c(OC)cc1N1CCC(N2CCN(CCC3CCN(c4cc(F)c(C5CCC(=O)NC5=O)c(F)c4)CC3)CC2)CC1]

The title compound was prepared in a procedure similar to that in Example 484. [M+H]⁺=992.8.

### Example 704: 3-(4-(3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)propoxy)-2,6-difluorophenyl)piperidine-2,6-dione

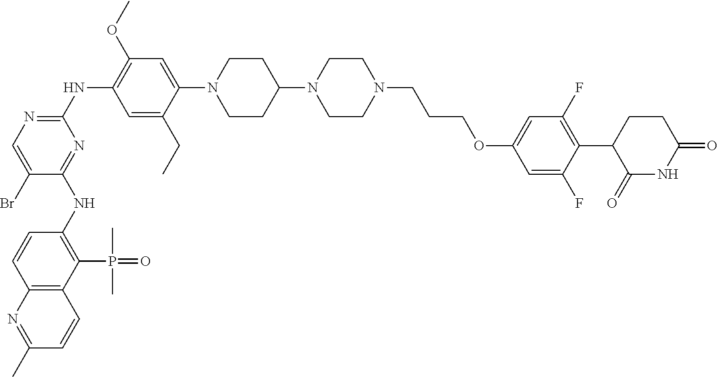 [SMILES: CCc1cc(Nc2ncc(Br)c(Nc3ccc4nc(C)ccc4c3P(C)(C)=O)n2)c(OC)cc1N1CCC(N2CCN(CCCOc3cc(F)c(C4CCC(=O)NC4=O)c(F)c3)CC2)CC1]

The title compound was prepared in a procedure similar to that in Example 484. [M+H]⁺=988.6.

### Example 705: 3-(4-(2-(4-(1-(4-((5-bromo-4-((5-(dimethylphosphoryl)-2-methyl-1-oxo-1,2-dihydroisoquinolin-6-yl)amino)pyrimidin-2-yl)amino)-2-ethyl-5-methoxyphenyl)piperidin-4-yl)piperazin-1-yl)ethyl)-3-methyl-2-oxo-2,3-dihydro-1H-benzo[d]imidazol-1-yl)piperidine-2,6-dione

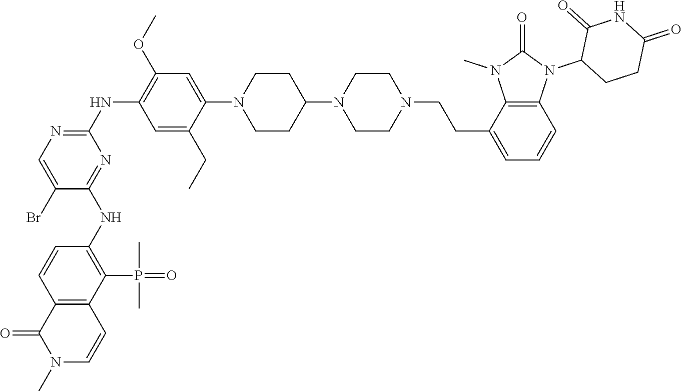 [SMILES: CCc1cc(Nc2ncc(Br)c(Nc3ccc4c(=O)n(C)ccc4c3P(C)(C)=O)n2)c(OC)cc1N1CCC(N2CCN(CCc3cccc4c3n(C)c(=O)n4C3CCC(=O)NC3=O)CC2)CC1]

The title compound was prepared in a procedure similar to that in Example 318. [M+H]⁺=1008.5.

### Example 706: 3-(4-((3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)-2,2-dimethyl-3-oxopropyl)amino)-2,6-difluorophenyl)piperidine-2,6-dione

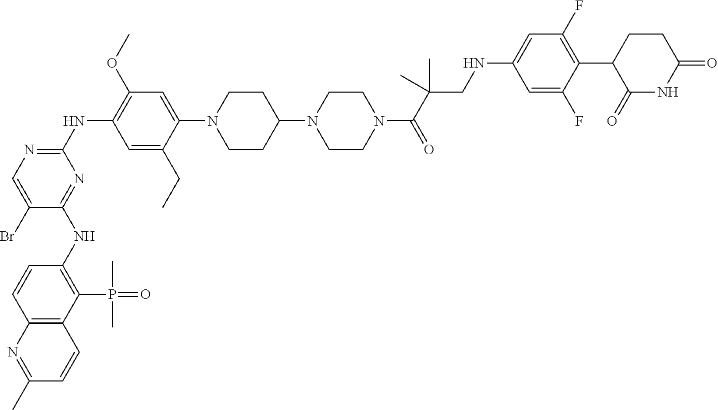 [SMILES: CCc1cc(Nc2ncc(Br)c(Nc3ccc4nc(C)ccc4c3P(C)(C)=O)n2)c(OC)cc1N1CCC(N2CCN(C(=O)C(C)(C)CNc3cc(F)c(C4CCC(=O)NC4=O)c(F)c3)CC2)CC1]

The title compound was prepared in a procedure similar to that in Example 488. [M+H]⁺=1029.4.

### Example 707: 3-(4-(2-(4-(1-(4-((5-bromo-4-((5-(dimethylphosphoryl)-2,3-dimethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

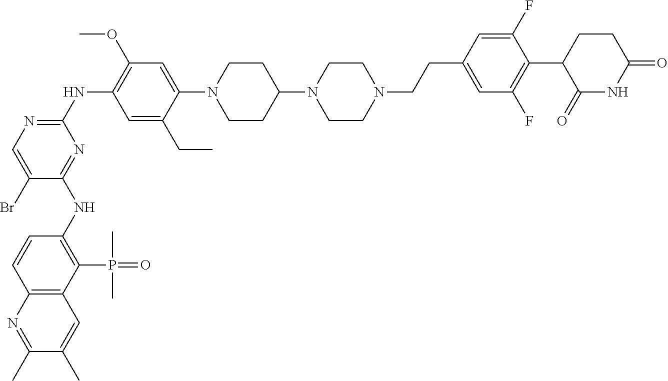 [SMILES: CCc1cc(Nc2ncc(Br)c(Nc3ccc4nc(C)c(C)cc4c3P(C)(C)=O)n2)c(OC)cc1N1CCC(N2CCN(CCc3cc(F)c(C4CCC(=O)NC4=O)c(F)c3)CC2)CC1]

The title compound was prepared in a procedure similar to that in Example 484. [M+H]⁺=972.34.

### Example 708: 3-(4-(2-(3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)azetidin-1-yl)ethyl)-2,6-difluorophenyl)piperidine-2,6-dione

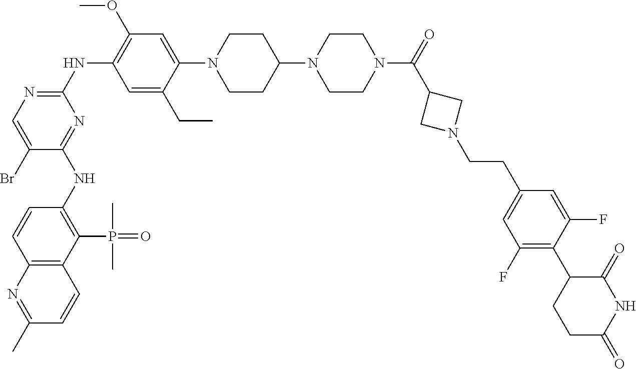 [SMILES: CCc1cc(Nc2ncc(Br)c(Nc3ccc4nc(C)ccc4c3P(C)(C)=O)n2)c(OC)cc1N1CCC(N2CCN(C(=O)C3CN(CCc4cc(F)c(C5CCC(=O)NC5=O)c(F)c4)C3)CC2)CC1]

The title compound was prepared in a procedure similar to that in Example 488. [M+H]⁺=1041.4.

### Example 709: 3-((4-(3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)cyclobutyl)phenyl)amino)piperidine-2,6-dione

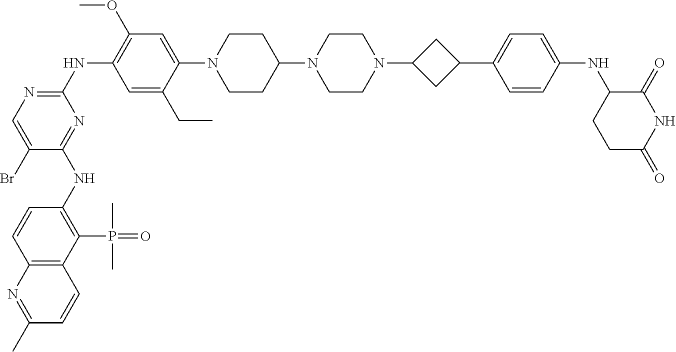 [SMILES: CCc1cc(Nc2ncc(Br)c(Nc3ccc4nc(C)ccc4c3P(C)(C)=O)n2)c(OC)cc1N1CCC(N2CCN(C3CC(c4ccc(NC5CCC(=O)NC5=O)cc4)C3)CC2)CC1]

The title compound was prepared in a procedure similar to that in Example 492. [M+H]⁺=965.8.

### Example 710: 3-(5′-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-2′-oxospiro[cyclopropane-1,3′-indolin]-1′-yl)piperidine-2,6-dione

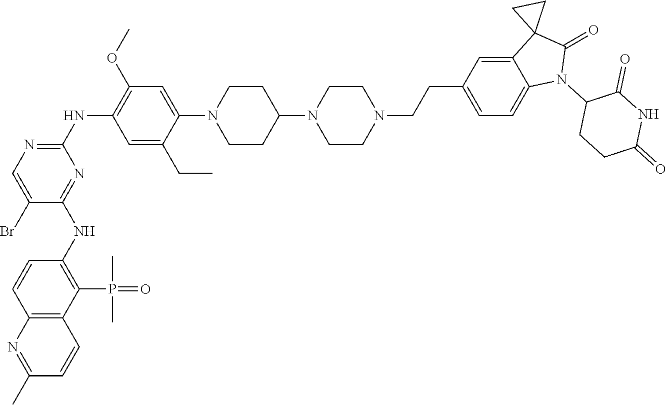 [SMILES: CCc1cc(Nc2ncc(Br)c(Nc3ccc4nc(C)ccc4c3P(C)(C)=O)n2)c(OC)cc1N1CCC(N2CCN(CCc3ccc4c(c3)C3(CC3)C(=O)N4C3CCC(=O)NC3=O)CC2)CC1]

The title compound was prepared in a procedure similar to that in Example 413. [M+H]⁺=1003.4.

### Example 711: 3-(5-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)-3,3-dimethyl-2-oxoindolin-1-yl)piperidine-2,6-dione

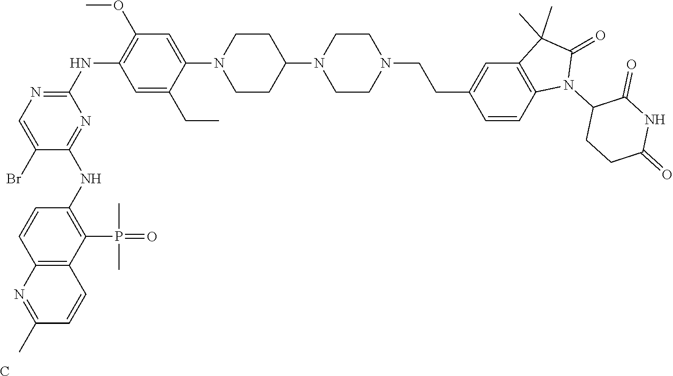

The title compound was prepared in a procedure similar to that in Example 413. ¹H NMR (500 MHz, DMSO) δ 11.70 (s, 1H), 10.99 (s, 1H), 8.49 (d, J=8.7 Hz, 1H), 8.21 (s, 1H), 8.14 (s, 1H), 7.91 (s, 1H), 7.80 (d, J=9.3 Hz, 1H), 7.36 (d, J=8.9 Hz, 1H), 7.31 (s, 1H), 7.18 (s, 1H), 7.00 (d, J=7.4 Hz, 1H), 6.80 (d, J=8.0 Hz, 1H), 6.67 (s, 1H), 5.12 (s, 1H), 3.69 (s, 3H), 2.87 (d, J=10.9 Hz, 4H), 2.68-2.61 (m, 3H), 2.60-2.54 (m, 7H), 2.49 (d, J=18.1 Hz, 4H), 2.40 (s, 4H), 2.23 (d, J=7.3 Hz, 3H), 1.91 (d, J=13.3 Hz, 7H), 1.77 (d, J=11.0 Hz, 2H), 1.48 (d, J=8.7 Hz, 2H), 1.21 (d, J=2.9 Hz, 6H), 0.70 (s, 3H). [M+H]⁺=1006.4

### Example 712: 3-(5-(3-(4-(1-(4-((5-bromo-4-((5-(dimethylphosphoryl)-3-methyl-4-oxo-3,4-dihydroquinazolin-6-yl)amino)pyrimidin-2-yl)amino)-2-ethyl-5-methoxyphenyl)piperidin-4-yl)piperazine-1-carbonyl)azetidin-1-yl)-1-oxoisoindolin-2-yl)piperidine-2,6-dione

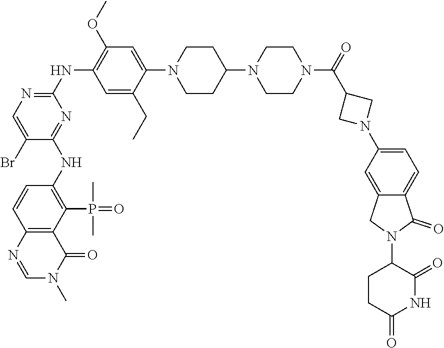 [SMILES: CCc1cc(Nc2ncc(Br)c(Nc3ccc4ncn(C)c(=O)c4c3P(C)(C)=O)n2)c(OC)cc1N1CCC(N2CCN(C(=O)C3CN(c4ccc5c(c4)CN(C4CCC(=O)NC4=O)C5=O)C3)CC2)CC1]

The title compound was prepared in a procedure similar to that in Example 447. [M+H]⁺=1049.3.

### Example 713: 3-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)-1,4-diazepan-1-yl)ethyl)-2,6-difluorophenyl)piperidine-2,6-dione

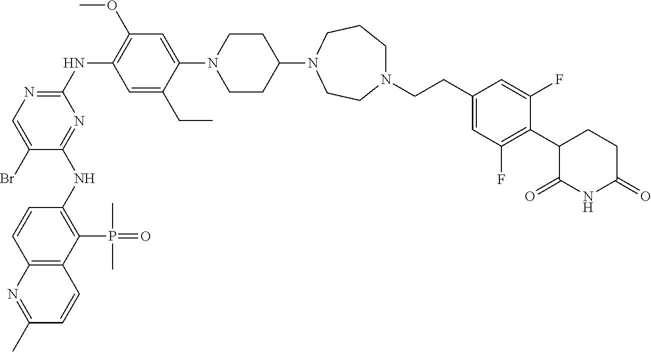 [SMILES: CCc1cc(Nc2ncc(Br)c(Nc3ccc4nc(C)ccc4c3P(C)(C)=O)n2)c(OC)cc1N1CCC(N2CCCN(CCc3cc(F)c(C4CCC(=O)NC4=O)c(F)c3)CC2)CC1]

The title compound was prepared in a procedure similar to that in Example 484. [M+H]⁺=972.3.

### Example 714: 3-(4-(2-(4-(1-(4-((5-bromo-4-((7-(dimethylphosphoryl)benzo[d]thiazol-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

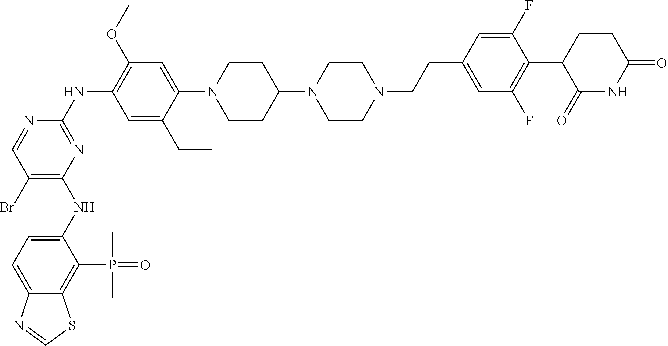 [SMILES: CCc1cc(Nc2ncc(Br)c(Nc3ccc4ncsc4c3P(C)(C)=O)n2)c(OC)cc1N1CCC(N2CCN(CCc3cc(F)c(C4CCC(=O)NC4=O)c(F)c3)CC2)CC1]

The title compound was prepared in a procedure similar to that in Example 484. [M+H]⁺=950.4.

### Example 715: 3-(4-(2-((S)-4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)-3-methylpiperazin-1-yl)ethyl)-2,6-difluorophenyl)piperidine-2,6-dione

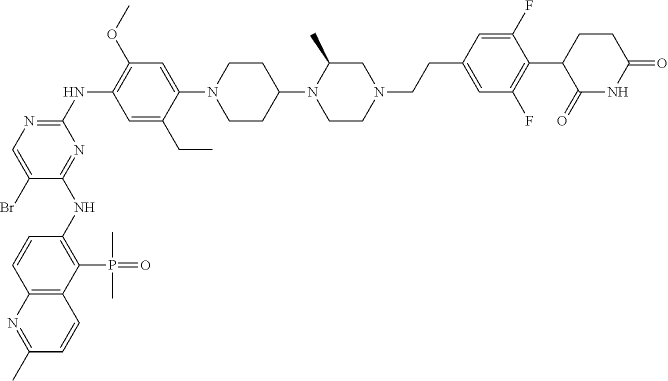 [SMILES: CCc1cc(Nc2ncc(Br)c(Nc3ccc4nc(C)ccc4c3P(C)(C)=O)n2)c(OC)cc1N1CCC(N2CCN(CCc3cc(F)c(C4CCC(=O)NC4=O)c(F)c3)C[C@@H]2C)CC1]

The title compound was prepared in a procedure similar to that in Example 484. [M+H]⁺=972.7.

### Example 716: 3-(4-(2-((2S,5R)-4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)-2,5-dimethylpiperazin-1-yl)ethyl)-2,6-difluorophenyl)piperidine-2,6-dione

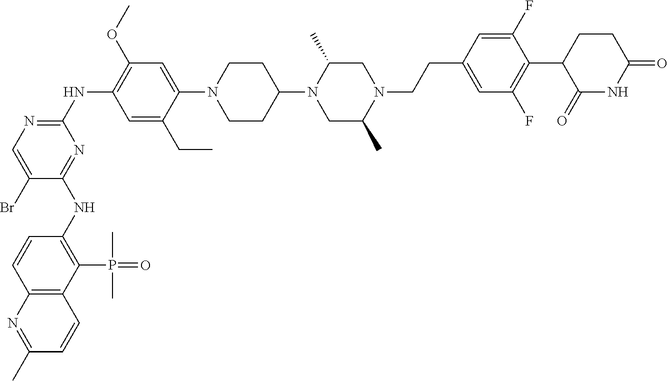 [SMILES: CCc1cc(Nc2ncc(Br)c(Nc3ccc4nc(C)ccc4c3P(C)(C)=O)n2)c(OC)cc1N1CCC(N2C[C@H](C)N(CCc3cc(F)c(C4CCC(=O)NC4=O)c(F)c3)C[C@H]2C)CC1]

The title compound was prepared in a procedure similar to that in Example 484. [M+H]⁺=986.5.

### Example 717: 3-(4-((1s,3s)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)cyclobutyl)-2,6-difluorophenyl)piperidine-2,6-dione

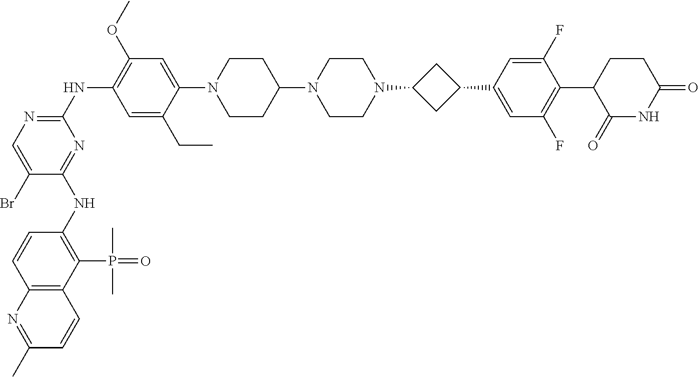 [SMILES: CCc1cc(Nc2ncc(Br)c(Nc3ccc4nc(C)ccc4c3P(C)(C)=O)n2)c(OC)cc1N1CCC(N2CCN([C@H]3C[C@@H](c4cc(F)c(C5CCC(=O)NC5=O)c(F)c4)C3)CC2)CC1]

The title compound was prepared in a procedure similar to that in Example 484. [M+H]⁺=984.5.

### Example 718: 3-((4-(4-((4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)methyl)piperidin-1-yl)-2,6-difluorophenyl)amino)piperidine-2,6-dione

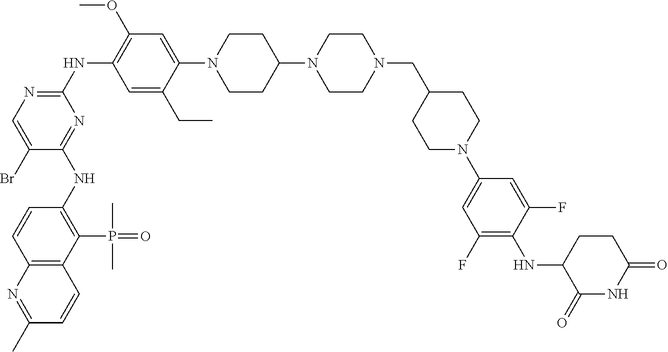 [SMILES: CCc1cc(Nc2ncc(Br)c(Nc3ccc4nc(C)ccc4c3P(C)(C)=O)n2)c(OC)cc1N1CCC(N2CCN(CC3CCN(c4cc(F)c(NC5CCC(=O)NC5=O)c(F)c4)CC3)CC2)CC1]

The title compound was prepared in a procedure similar to that in Example 484. [M+H]⁺=1042.1.

### Example 719: 3-(5-(4-(2-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazin-1-yl)ethyl)piperidin-1-yl)-2H-indazol-2-yl)piperidine-2,6-dione

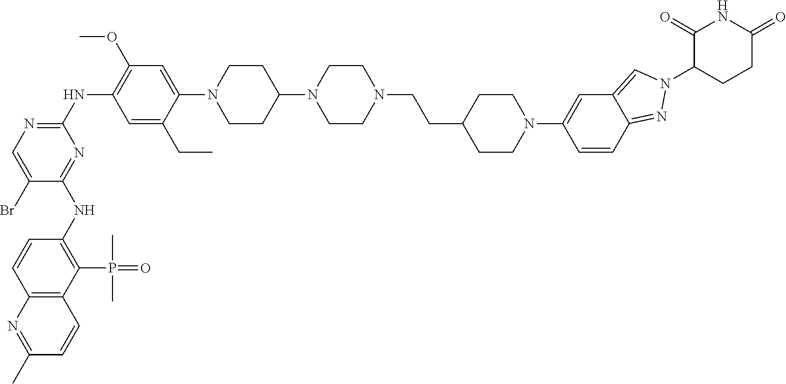 [SMILES: CCc1cc(Nc2ncc(Br)c(Nc3ccc4nc(C)ccc4c3P(C)(C)=O)n2)c(OC)cc1N1CCC(N2CCN(CCC3CCN(c4ccc5nn(C6CCC(=O)NC6=O)cc5c4)CC3)CC2)CC1]

The title compound was prepared in a procedure similar to that in Example 492. [M+H]⁺=1045.4.

### Example 720: 3-(4-(4-(2-(4-(1-(4-((5-bromo-4-((5-(dimethylphosphoryl)-2-methyl-1-oxo-1,2-dihydroisoquinolin-6-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

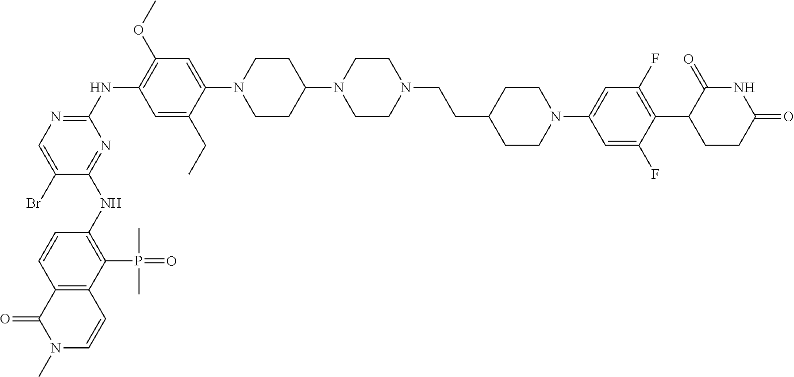 [SMILES: CCc1cc(Nc2ncc(Br)c(Nc3ccc4c(=O)n(C)ccc4c3P(C)(C)=O)n2)c(OC)cc1N1CCC(N2CCN(CCC3CCN(c4cc(F)c(C5CCC(=O)NC5=O)c(F)c4)CC3)CC2)CC1]

The title compound was prepared in a procedure similar to that in Example 484. [M+H]⁺=1057.7.

### Example 721: 3-(4-(4-(2-(4-(1-(4-((5-bromo-4-((8-(dimethylphosphoryl)-3-methyl-4-oxo-3,4-dihydroquinazolin-7-yl)amino)pyrimidin-2-yl)amino)-2-ethyl-5-methoxyphenyl)piperidin-4-yl)piperazin-1-yl)ethyl)piperidin-1-yl)-2,6-difluorophenyl)piperidine-2,6-dione

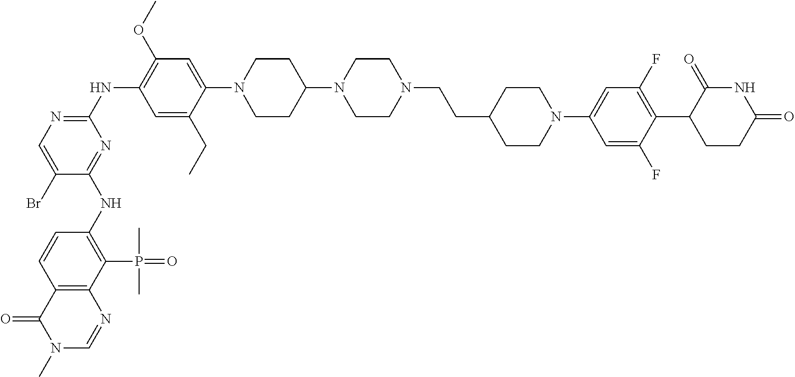 [SMILES: CCc1cc(Nc2ncc(Br)c(Nc3ccc4c(=O)n(C)cnc4c3P(C)(C)=O)n2)c(OC)cc1N1CCC(N2CCN(CCC3CCN(c4cc(F)c(C5CCC(=O)NC5=O)c(F)c4)CC3)CC2)CC1]

The title compound was prepared in a procedure similar to that in Example 484. [M+H]⁺=1058.7.

### Example 723: 5-(3-(4-(4-((5-bromo-4-((5-(dimethylphosphoryl)quinoxalin-6-yl)amino)pyrimidin-2-yl)amino)-5-methoxy-2-methylphenyl)piperazin-1-yl)azetidine-1-carbonyl)-N-(4-(2,6-dioxopiperidin-3-yl)phenyl)pyrazine-2-carboxamide

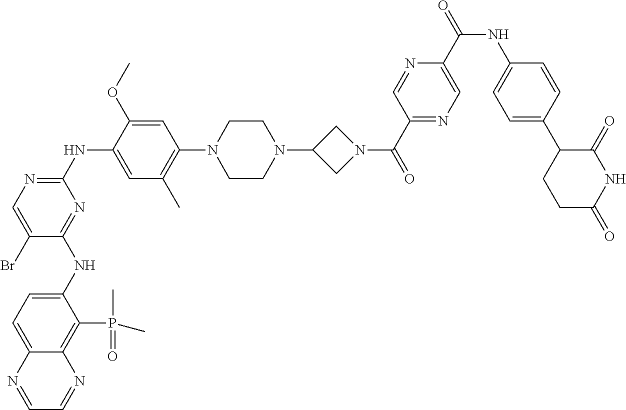 [SMILES: COc1cc(N2CCN(C3CN(C(=O)c4cnc(C(=O)Nc5ccc(C6CCC(=O)NC6=O)cc5)cn4)C3)CC2)c(C)cc1Nc1ncc(Br)c(Nc2ccc3nccnc3c2P(C)(C)=O)n1]

The title compound was prepared in a procedure similar to that in Example 488. [M+H]⁺=988.5.

### Example 724: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-2-oxo-6-(trifluoromethyl)benzo[d]oxazol-3(2H)-yl)piperidine-2,6-dione

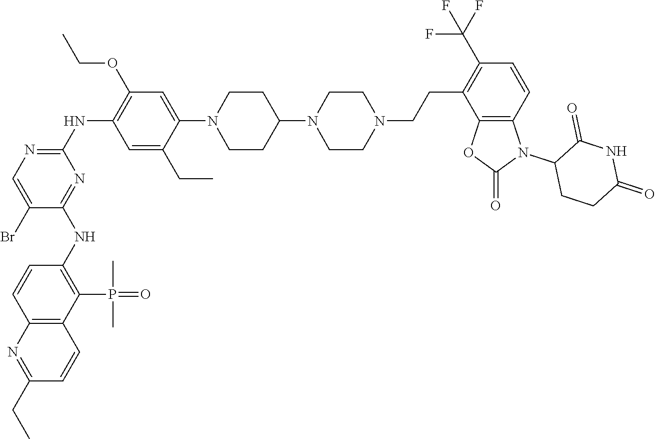 [SMILES: CCOc1cc(N2CCC(N3CCN(CCc4c(C(F)(F)F)ccc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1074.2.

### Example 726: 3-(7-(3-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)propyl)-5-chloro-2-oxobenzo[d]oxazol-3(2H)-yl)piperidine-2,6-dione

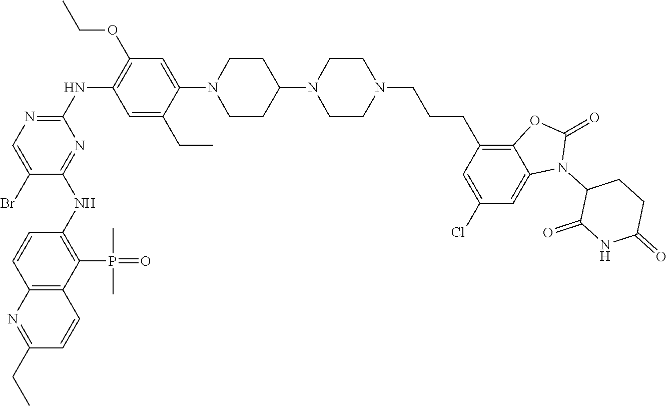 [SMILES: CCOc1cc(N2CCC(N3CCN(CCCc4cc(Cl)cc5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. [M+H]⁺=1055.0.

### Example 727: (R)-3-(4-((R)-4-(4-(1-(4-((5-bromo-4-((5-(dimethylphosphoryl)-2-methylquinolin-6-yl)amino)pyrimidin-2-yl)amino)-5-ethoxy-2-methylphenyl)piperidin-4-yl)piperazine-1-carbonyl)-3,3-dimethylpyrrolidin-1-yl)-2,6-difluorophenyl)piperidine-2,6-dione

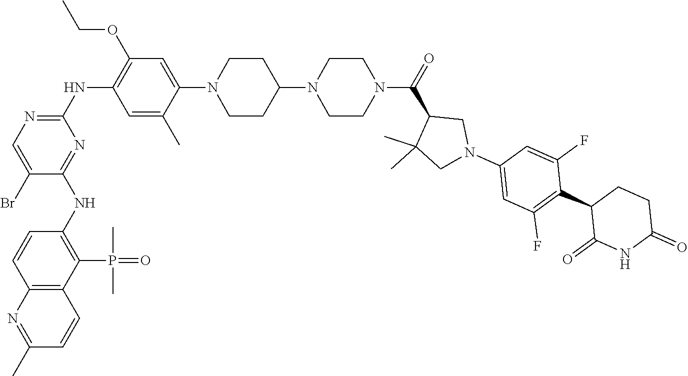 [SMILES: CCOc1cc(N2CCC(N3CCN(C(=O)[C@H]4CN(c5cc(F)c([C@H]6CCC(=O)NC6=O)c(F)c5)CC4(C)C)CC3)CC2)c(C)cc1Nc1ncc(Br)c(Nc2ccc3nc(C)ccc3c2P(C)(C)=O)n1]

The title compound was prepared in a procedure similar to that in Example 488. ¹H NMR (500 MHz, DMSO) δ 11.87 (s, 1H), 10.84 (s, 1H), 8.55 (d, J=8.9 Hz, 1H), 8.37 (d, J=5.5 Hz, 1H), 8.22 (s, 1H), 7.93-7.84 (m, 2H), 7.49-7.37 (m, 2H), 6.65 (s, 1H), 6.15 (d, J=12.2 Hz, 2H), 4.01 (q, J=7.0 Hz, 3H), 3.64-3.38 (m, 7H), 3.12-2.98 (m, 4H), 2.74-2.82 (m, 1H), 2.65 (s, 3H), 2.54-2.61 (m, 7H), 2.36 (s, 1H), 2.14-2.03 (m, 1H), 2.03-1.92 (m, 7H), 1.92-1.78 (m, 5H), 1.58 (q, J=12.4 Hz, 2H), 1.26 (t, J=6.9 Hz, 3H), 1.18 (s, 3H), 0.99 (s, 3H). [M+H]⁺=1055.0.

### Example 728: (R)-3-(4-(2-(4-(1-(4-((5-bromo-4-((5-(dimethylphosphoryl)-3-fluoro-2-methylquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

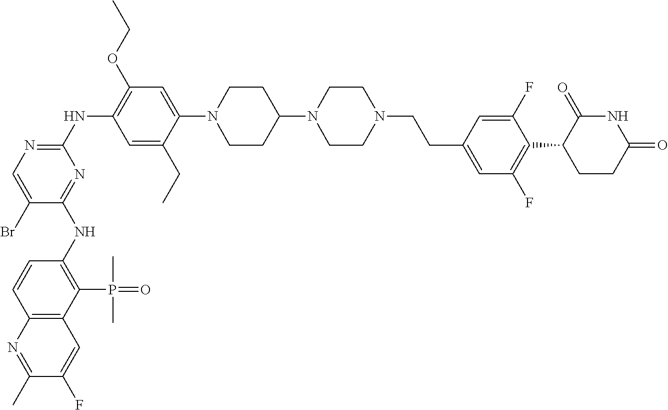 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(C)c(F)cc3c2P(C)(C)=O)n1]

The title compound was prepared in a procedure similar to that in Example 484. ¹H NMR (500 MHz, DMSO) δ 11.19 (s, 1H), 10.95 (s, 1H), 8.69 (d, J=12.0 Hz, 1H), 8.23 (s, 1H), 8.15 (s, 1H), 7.95 (d, J=9.1 Hz, 1H), 7.85 (s, 1H), 7.37 (s, 1H), 7.02 (d, J=10.1 Hz, 2H), 6.68 (s, 1H), 4.20 (dd, J=12.7, 4.9 Hz, 1H), 3.99 (d, J=6.9 Hz, 2H), 2.90-2.73 (m, 6H), 2.66-2.53 (m, 11H), 2.48-2.40 (m, 3H), 2.26 (s, 1H), 2.17-2.08 (m, 3H), 2.02-1.92 (m, 8H), 1.81 (d, J=10.9 Hz, 2H), 1.56-1.47 (m, 2H), 1.26 (t, J=6.9 Hz, 3H), 0.64 (s, 3H). [M+H]⁺=990.5.

### Example 729: (R)-3-(4-((R)-4-(4-(1-(4-((5-bromo-4-((5-(dimethylphosphoryl)-3-fluoro-2-methylquinolin-6-yl)amino)pyrimidin-2-yl)amino)-5-methoxy-2-methylphenyl)piperidin-4-yl)piperazine-1-carbonyl)-3,3-dimethylpyrrolidin-1-yl)-2,6-difluorophenyl)piperidine-2,6-dione

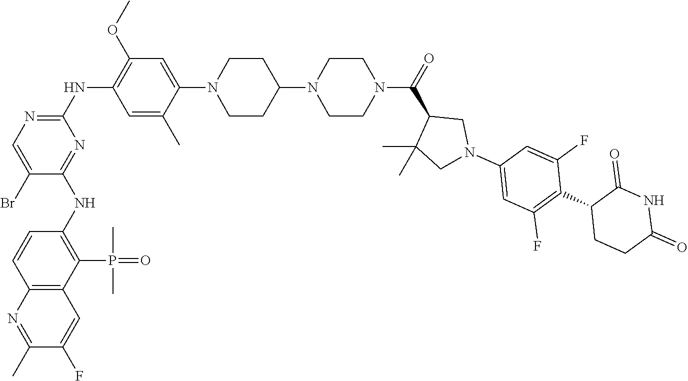 [SMILES: COc1cc(N2CCC(N3CCN(C(=O)[C@H]4CN(c5cc(F)c([C@H]6CCC(=O)NC6=O)c(F)c5)CC4(C)C)CC3)CC2)c(C)cc1Nc1ncc(Br)c(Nc2ccc3nc(C)c(F)cc3c2P(C)(C)=O)n1]

The title compound was prepared in a procedure similar to that in Example 488. ¹H NMR (500 MHz, DMSO) δ 11.51 (s, 1H), 10.84 (s, 1H), 8.53 (d, J=12.0 Hz, 1H), 8.26 (s, 1H), 8.22 (s, 1H), 7.95 (d, J=9.0 Hz, 2H), 7.30 (s, 1H), 6.66 (s, 1H), 6.15 (d, J=12.2 Hz, 2H), 4.01 (dd, J=12.7, 4.8 Hz, 1H), 3.75 (s, 3H), 3.60 (s, 2H), 3.53-3.38 (m, 6H), 3.09 (dd, J=18.4, 9.1 Hz, 2H), 3.00 (d, J=10.7 Hz, 2H), 2.83-2.74 (m, 1H), 2.66-2.54 (m, 8H), 2.36 (s, 1H), 2.07 (t, J=12.7 Hz, 1H), 1.99-1.93 (m, 8H), 1.86-1.80 (m, 5H), 1.57 (d, J=11.4 Hz, 2H), 1.18 (s, 3H), 0.99 (s, 3H). [M+H]⁺=1059.6.

### Example 730: (R)-3-(4-(2-(4-(1-(4-((5-chloro-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

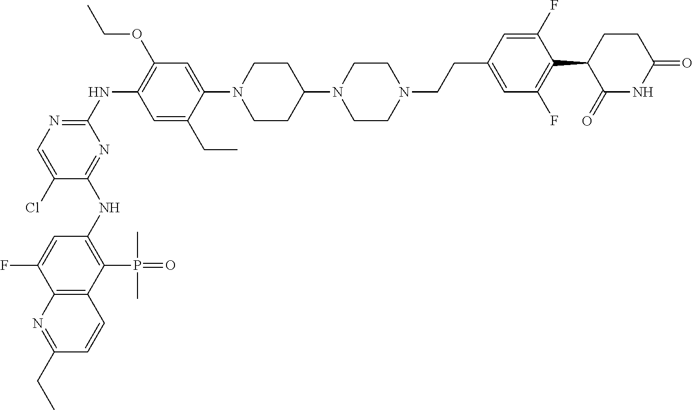 [SMILES: CCOc1cc(N2CCC(N3CCN(CCc4cc(F)c([C@H]5CCC(=O)NC5=O)c(F)c4)CC3)CC2)c(CC)cc1Nc1ncc(Cl)c(Nc2cc(F)c3nc(CC)ccc3c2P(C)(C)=O)n1]

The title compound was prepared in a procedure similar to that in Example 484. ¹H NMR (500 MHz, DMSO) δ 12.43 (s, 1H), 10.95 (s, 1H), 8.53 (d, J=8.8 Hz, 2H), 8.17 (s, 1H), 8.10 (s, 1H), 7.54 (d, J=9.0 Hz, 1H), 7.39 (s, 1H), 7.03 (d, J=10.0 Hz, 2H), 6.73 (s, 1H), 4.20 (dd, J=12.8, 5.3 Hz, 1H), 4.02 (q, J=6.9 Hz, 2H), 3.01-2.91 (m, 4H), 2.85-2.71 (m, 4H), 2.66-2.64 (m, 5H), 2.56-2.53 (m, 6H), 2.40-2.33 (m, 2H), 2.30-2.28 (m, 1H), 2.18-2.07 (m, 2H), 2.01-1.99 (m, 7H), 1.88-1.81 (m, 2H), 1.60-1.49 (m, 2H), 1.32 (t, J=7.6 Hz, 3H), 1.25 (t, J=6.9 Hz, 3H), 0.85-0.82 (m, 3H). [M+H]⁺=960.3.

### Example 731: 3-(7-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-4,6-difluoro-2-oxobenzo[d]oxazol-3(2H)-yl)piperidine-2,6-dione

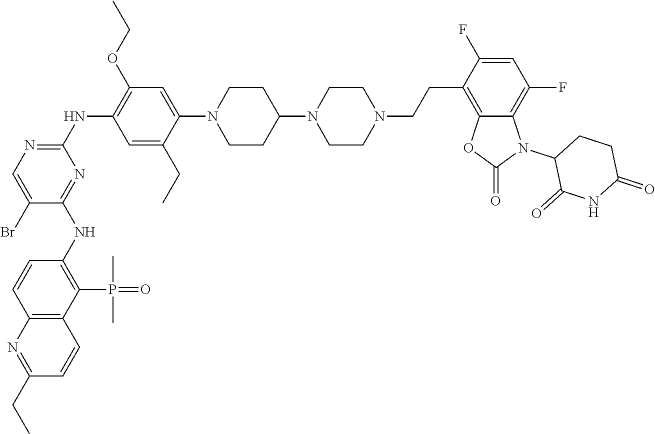 [SMILES: CCOc1cc(N2CCC(N3CCN(CCc4c(F)cc(F)c5c4oc(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 492. ¹H NMR (500 MHz, DMSO) δ 11.72 (s, 1H), 11.26 (s, 1H), 8.60 (d, J=8.8 Hz, 1H), 8.22 (s, 2H), 7.91 (s, 1H), 7.87 (d, J=9.3 Hz, 1H), 7.45 (d, J=8.9 Hz, 1H), 7.38 (s, 1H), 7.27 (t, J=10.9 Hz, 1H), 6.70 (s, 1H), 5.43 (s, 1H), 4.00 (d, J=7.0 Hz, 2H), 2.98-2.83 (m, 8H), 2.70-2.52 (m, 8H), 2.38-2.34 (m, 4H), 2.32-2.16 (m, 5H), 1.98 (d, J=13.3 Hz, 6H), 1.82 (d, J=10.7 Hz, 2H), 1.56-1.48 (m, 2H), 1.32 (t, J=7.6 Hz, 3H), 1.25 (t, J=6.9 Hz, 3H), 0.71 (s, 3H). [M+H]⁺=1043.5.

### Example 732: (R)-3-(4-((R)-3-(4-(1-(4-((5-bromo-4-((5-(dimethylphosphoryl)-3-fluoro-2-methylquinolin-6-yl)amino)pyrimidin-2-yl)amino)-5-methoxy-2-methylphenyl)piperidin-4-yl)piperazine-1-carbonyl)pyrrolidin-1-yl)-2,6-difluorophenyl)piperidine-2,6-dione

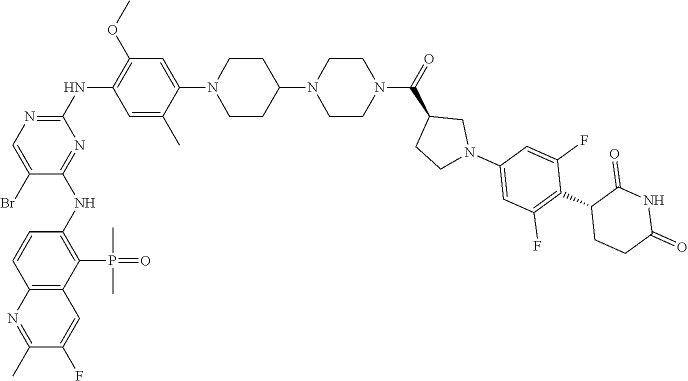 [SMILES: COc1cc(N2CCC(N3CCN(C(=O)[C@@H]4CCN(c5cc(F)c([C@H]6CCC(=O)NC6=O)c(F)c5)C4)CC3)CC2)c(C)cc1Nc1ncc(Br)c(Nc2ccc3nc(C)c(F)cc3c2P(C)(C)=O)n1]

The title compound was prepared in a procedure similar to that in Example 488. ¹H NMR (500 MHz, DMSO) δ 11.50 (s, 1H), 10.84 (s, 1H), 8.53 (d, J=12.4 Hz, 1H), 8.27 (s, 1H), 8.22 (s, 1H), 7.95 (d, J=11.9 Hz, 2H), 7.30 (s, 1H), 6.67 (s, 1H), 6.23 (d, J=12.1 Hz, 2H), 4.02 (dd, J=12.4, 5.0 Hz, 1H), 3.75 (s, 3H), 3.60-3.41 (m, 8H), 3.29-3.22 (m, 3H), 3.01 (d, J=10.7 Hz, 2H), 2.82-2.73 (m, 1H), 2.66-2.54 (m, 8H), 2.36 (s, 1H), 2.19-2.04 (m, 3H), 1.99-1.93 (m, 7H), 1.87-1.80 (m, 5H), 1.58 (d, J=9.8 Hz, 2H). [M+H]⁺=1031.5.

### Example 733: (R)-3-(4-(2-(4-(1-(4-((4-((5-(dimethylphosphoryl)-2-ethyl-8-fluoroquinolin-6-yl)amino)-5-methylpyrimidin-2-yl)amino)-5-ethoxy-2-ethylphenyl)piperidin-4-yl)piperazin-1-yl)ethyl)-2,6-difluorophenyl)piperidine-2,6-dione

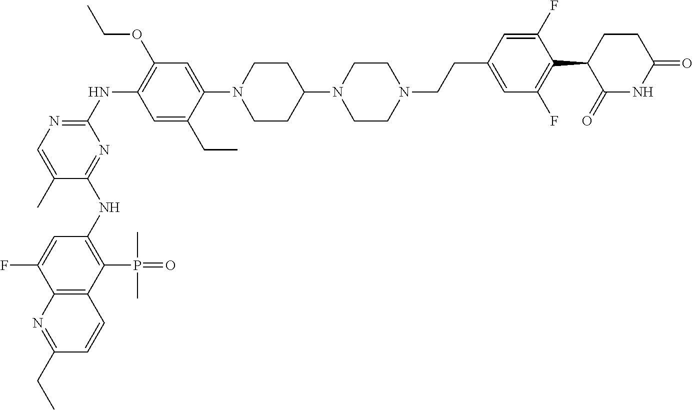 [SMILES: CCOc1cc(N2CCC(N3CCN(CCc4cc(F)c([C@H]5CCC(=O)NC5=O)c(F)c4)CC3)CC2)c(CC)cc1Nc1ncc(C)c(Nc2cc(F)c3nc(CC)ccc3c2P(C)(C)=O)n1]

The title compound was prepared in a procedure similar to that in Example 486. ¹H NMR (500 MHz, DMSO) δ 12.17 (s, 1H), 10.95 (s, 1H), 8.79 (d, J=14.1 Hz, 1H), 8.46 (d, J=9.5 Hz, 1H), 7.97 (s, 1H), 7.67 (s, 1H), 7.62 (s, 1H), 7.53 (d, J=9.0 Hz, 1H), 7.03 (d, J=10.0 Hz, 2H), 6.74 (s, 1H), 4.20 (dd, J=12.6, 5.0 Hz, 1H), 4.05 (q, J=7.0 Hz, 2H), 2.99-2.91 (m, 4H), 2.86-2.71 (m, 3H), 2.65-2.62 (m, 3H), 2.58-2.55 (m, 7H), 2.48-2.35 (m, 5H), 2.29-2.26 (m, 1H), 2.19-2.08 (m, 4H), 2.03-2.01 (m, 7H), 1.86-1.83 (m, 2H), 1.56-1.54 (m, 2H), 1.34-1.27 (m, 6H), 0.85 (t, J=7.4 Hz, 3H). [M+H]⁺=940.3.

### Example 734: (R)-3-(4-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)propoxy)-2,6-difluorophenyl)piperidine-2,6-dione

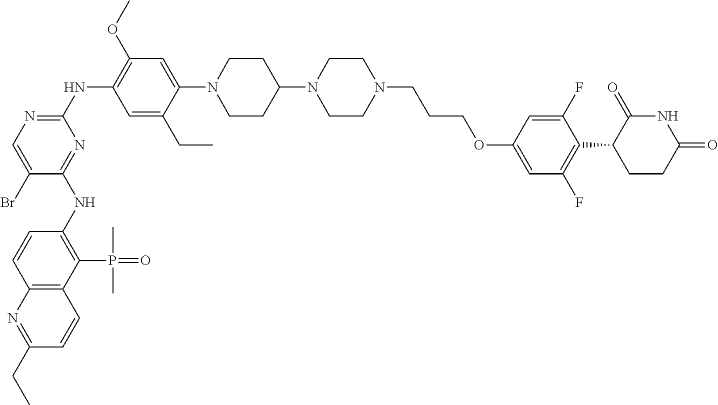 [SMILES: CCc1ccc2c(P(C)(C)=O)c(Nc3nc(Nc4cc(CC)c(N5CCC(N6CCN(CCCOc7cc(F)c([C@H]8CCC(=O)NC8=O)c(F)c7)CC6)CC5)cc4OC)ncc3Br)ccc2n1]

The title compound was prepared in a procedure similar to that in Example 486. [M+H]⁺=1002.2.

### Example 735: (R)-3-(4-(3-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)propoxy)-2,6-difluorophenyl)piperidine-2,6-dione

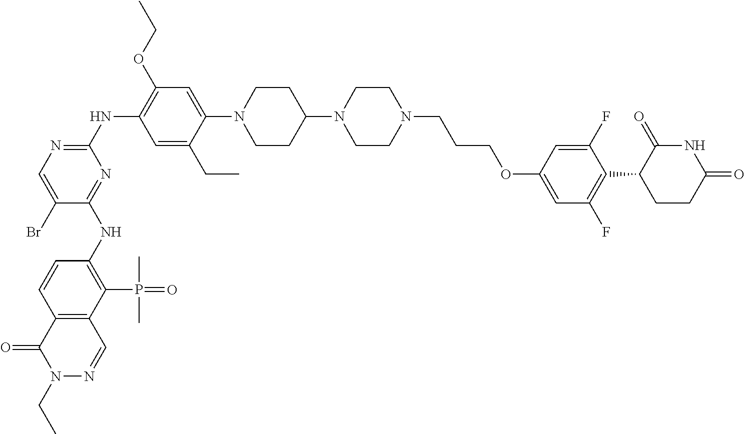 [SMILES: CCOc1cc(N2CCC(N3CCN(CCCOc4cc(F)c([C@H]5CCC(=O)NC5=O)c(F)c4)CC3)CC2)c(CC)cc1Nc1ncc(Br)c(Nc2ccc3c(=O)n(CC)ncc3c2P(C)(C)=O)n1]

The title compound was prepared in a procedure similar to that in Example 486. [M+H]⁺=1033.4.

### Example 736: 3-(4-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-5-fluoro-3-methyl-2-oxo-2,3-dihydro-1H-benzol[d]imidazol-1-yl)piperidine-2,6-dione

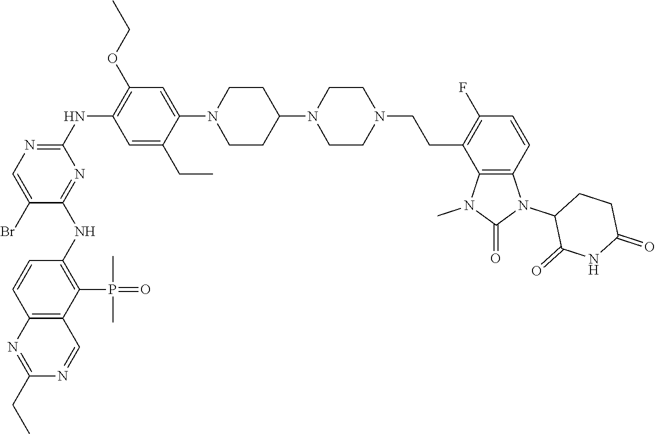 [SMILES: CCOc1cc(N2CCC(N3CCN(CCc4c(F)ccc5c4n(C)c(=O)n5C4CCC(=O)NC4=O)CC3)CC2)c(CC)cc1Nc1ncc(Br)c(Nc2ccc3nc(CC)ncc3c2P(C)(C)=O)n1]

The title compound was prepared in a procedure similar to that in Example 318. ¹H NMR (500 MHz, DMSO) δ 11.46 (s, 1H), 11.11 (s, 1H), 9.94 (s, 1H), 8.42 (s, 1H), 8.26 (s, 1H), 7.94 (s, 1H), 7.87 (d, J=8.8 Hz, 1H), 7.35 (s, 1H), 7.02 (s, 1H), 6.91 (t, J=9.9 Hz, 1H), 6.71 (s, 1H), 5.38 (dd, J=12.7, 5.0 Hz, 1H), 4.01 (q, J=6.9 Hz, 2H), 3.60 (s, 3H), 3.31-3.28 (m, 2H), 3.08-3.04 (m, 5H), 2.89-2.86 (m, 4H), 2.68-2.62 (m, 8H), 2.49-2.45 (m, 5H), 2.20 (s, 2H), 2.04-2.01 (m, 8H), 1.62-1.61 (m, 1H), 1.39 (t, J=7.6 Hz, 3H), 1.25 (dd, J=13.4, 6.5 Hz, 3H), 0.68 (s, 3H). [M+H]⁺=1039.4.

### Example 737: 3-(4-(2-(4-(1-(4-((5-bromo-4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-3-ethyl-2-oxo-2,3-dihydro-1H-benzo[d]imidazol-1-yl)piperidine-2,6-dione

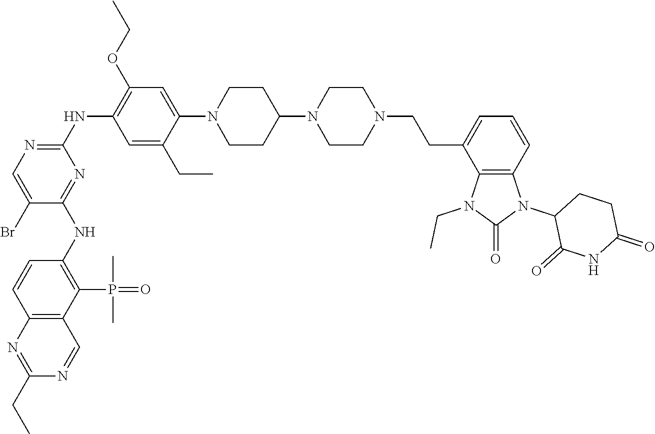 [SMILES: CCOc1cc(N2CCC(N3CCN(CCc4cccc5c4n(CC)c(=O)n5C4CCC(=O)NC4=O)CC3)CC2)c(CC)cc1Nc1ncc(Br)c(Nc2ccc3nc(CC)ncc3c2P(C)(C)=O)n1]

The title compound was prepared in a procedure similar to that in Example 318. ¹H NMR (500 MHz, DMSO) δ 11.46 (s, 1H), 11.10 (s, 1H), 9.94 (s, 1H), 8.42 (s, 1H), 8.26 (s, 1H), 7.95 (s, 1H), 7.87 (d, J=9.4 Hz, 1H), 7.34 (s, 1H), 7.01 (s, 2H), 6.94 (dd, J=5.9, 3.0 Hz, 1H), 6.71 (s, 1H), 5.37 (dd, J=12.8, 5.3 Hz, 1H), 4.01-3.96 (m, 4H), 3.30-3.26 (m, 2H), 3.13-2.84 (m, 10H), 2.79-2.57 (m, 8H), 2.50-2.47 (m, 2H), 2.19-2.16 (m, 2H), 1.99-1.85 (m, 9H), 1.62-1.58 (m, 2H), 1.39 (t, J=7.6 Hz, 3H), 1.25-1.13 (m, 6H), 0.68 (s, 3H). [M+H]⁺=1035.4.

### Example 738: (R)-3-(4-(2-(4-(1-(4-((5-bromo-4-((5-(dimethylphosphoryl)-2-methylquinazolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

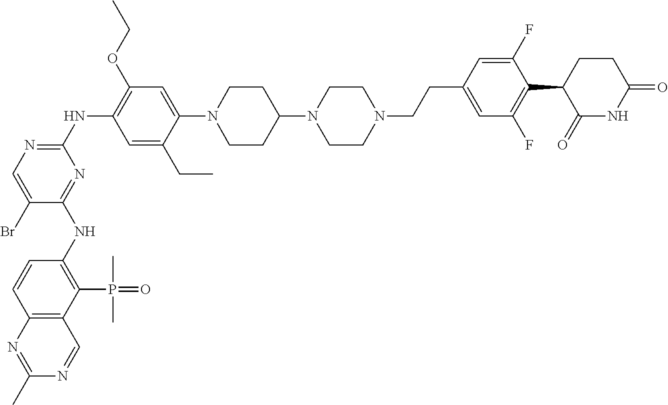 [SMILES: CCOc1cc(N2CCC(N3CCN(CCc4cc(F)c([C@H]5CCC(=O)NC5=O)c(F)c4)CC3)CC2)c(CC)cc1Nc1ncc(Br)c(Nc2ccc3nc(C)ncc3c2P(C)(C)=O)n1]

The title compound was prepared in a procedure similar to that in Example 460. ¹H NMR (500 MHz, DMSO) δ 11.44 (s, 1H), 10.95 (s, 1H), 9.91 (s, 1H), 8.43 (s, 1H), 8.25 (s, 1H), 7.93 (s, 1H), 7.86 (d, J=9.2 Hz, 1H), 7.36 (s, 1H), 7.04 (d, J=10.1 Hz, 2H), 6.69 (s, 1H), 4.20 (dd, J=12.7, 5.0 Hz, 1H), 4.00 (q, J=6.9 Hz, 2H), 3.31-3.28 (m, 2H), 2.91 (d, J=9.5 Hz, 2H), 2.86-2.70 (m, 7H), 2.68-2.51 (m, 9H), 2.36 (s, 2H), 2.15-2.11 (m, 3H), 2.01-1.92 (m, 7H), 1.86 (s, 2H), 1.56-1.51 (m, 2H), 1.25 (t, J=6.9 Hz, 3H), 0.68 (s, 3H). [M+H]⁺=973.3.

### Example 739: (R)-3-(4-(2-(4-(1-(4-((5-bromo-4-((5-(dimethylphosphoryl)-2-methylquinazolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

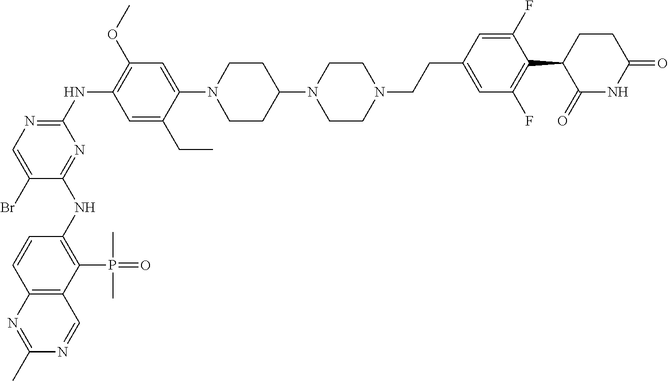 [SMILES: CCc1cc(Nc2ncc(Br)c(Nc3ccc4nc(C)ncc4c3P(C)(C)=O)n2)c(OC)cc1N1CCC(N2CCN(CCc3cc(F)c([C@H]4CCC(=O)NC4=O)c(F)c3)CC2)CC1]

The title compound was prepared in a procedure similar to that in Example 460. ¹H NMR (500 MHz, DMSO) δ 11.46 (s, 1H), 10.88 (s, 1H), 9.79 (s, 1H), 8.40 (s, 1H), 8.17 (s, 1H), 7.95 (s, 1H), 7.78 (d, J=9.2 Hz, 1H), 7.24 (s, 1H), 6.96 (d, J=10.1 Hz, 2H), 6.66 (s, 1H), 4.13 (dd, J=12.7, 4.9 Hz, 1H), 3.68 (s, 3H), 3.25-3.09 (m, 2H), 2.85 (d, J=10.6 Hz, 2H), 2.79-2.65 (m, 6H), 2.57 (t, J=11.1 Hz, 2H), 2.47-2.32 (m, 6H), 2.39-2.36 (m, 3H), 2.19 (s, 3H), 2.06 (dt, J=13.3, 9.7 Hz, 1H), 1.94-1.86 (m, 7H), 1.76 (d, J=10.9 Hz, 2H), 1.47-1.42 (m, 2H), 0.68 (s, 3H). [M+H]⁺=959.3.

### Example 740: (R)-3-(4-((R)-4-(4-(1-(4-((5-bromo-4-((5-(dimethylphosphoryl)-2-methylquinazolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

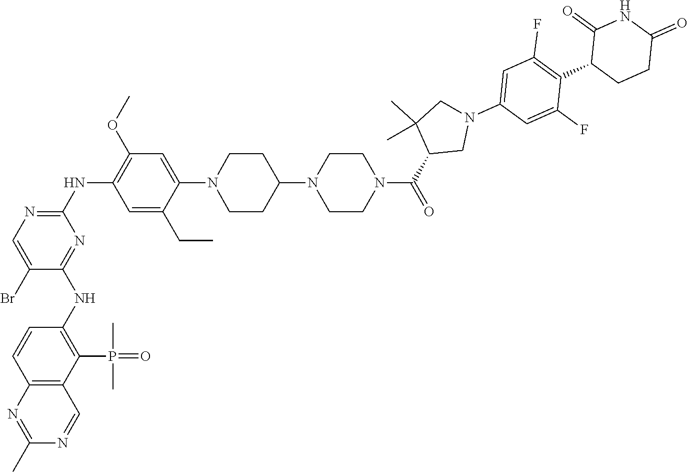 [SMILES: CCc1cc(Nc2ncc(Br)c(Nc3ccc4nc(C)ncc4c3P(C)(C)=O)n2)c(OC)cc1N1CCC(N2CCN(C(=O)[C@H]3CN(c4cc(F)c([C@H]5CCC(=O)NC5=O)c(F)c4)CC3(C)C)CC2)CC1]

The title compound was prepared in a procedure similar to that in Example 488. ¹H NMR (500 MHz, DMSO) δ 11.53 (s, 1H), 10.84 (s, 1H), 9.86 (s, 1H), 8.47 (s, 1H), 8.24 (s, 1H), 8.02 (s, 1H), 7.85 (d, J=8.2 Hz, 1H), 7.32 (s, 1H), 6.73 (s, 1H), 6.15 (d, J=12.2 Hz, 2H), 4.01 (dd, J=12.5, 4.9 Hz, 1H), 3.75 (s, 3H), 3.47-3.21 (m, 9H), 3.09-3.02 (m, 2H), 2.93 (d, J=10.3 Hz, 2H), 2.84-2.73 (m, 4H), 2.65 (t, J=10.8 Hz, 2H), 2.54-2.49 (m, 3H), 2.36-2.31 (m, 1H), 2.26-2.21 (m, 2H), 2.14-1.92 (m, 8H), 1.83 (d, J=10.4 Hz, 2H), 1.56 (d, J=10.6 Hz, 2H), 1.18 (s, 3H), 0.99 (s, 3H), 0.75 (s, 3H). [M+H]⁺=1056.4.

### Example 741: (R)-3-(4-(2-(4-(1-(4-((5-bromo-4-((2-cyclopropyl-5-(dimethylphosphoryl)quinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

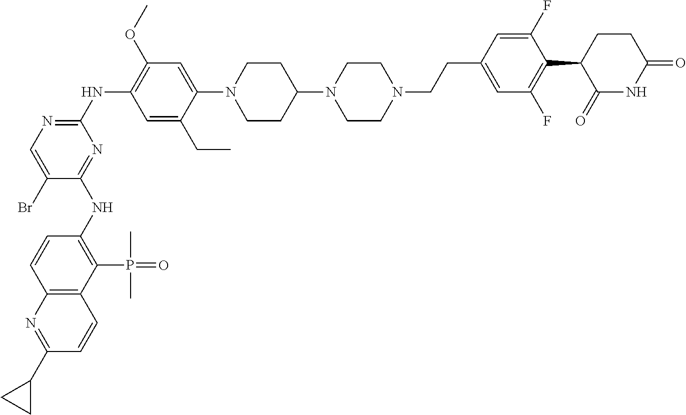 [SMILES: CCc1cc(Nc2ncc(Br)c(Nc3ccc4nc(C5CC5)ccc4c3P(C)(C)=O)n2)c(OC)cc1N1CCC(N2CCN(CCc3cc(F)c([C@H]4CCC(=O)NC4=O)c(F)c3)CC2)CC1]

The title compound was prepared in a procedure similar to that in Example 486. ¹H NMR (500 MHz, DMSO) δ 11.75 (s, 1H), 10.95 (s, 1H), 8.49 (d, J=8.9 Hz, 1H), 8.21-8.17 (m, 2H), 8.03 (s, 1H), 7.74 (d, J=9.4 Hz, 1H), 7.44 (d, J=9.0 Hz, 1H), 7.27 (s, 1H), 7.03 (d, J=10.0 Hz, 2H), 6.76 (s, 1H), 4.20 (dd, J=12.6, 5.0 Hz, 1H), 3.76 (s, 3H), 3.29-3.24 (m, 2H), 2.95 (d, J=10.9 Hz, 2H), 2.86-2.72 (m, 3H), 2.67 (t, J=11.2 Hz, 2H), 2.54-2.38 (m, 7H), 2.48-2.39 (m, 2H), 2.34-2.23 (m, 4H), 2.13 (dt, J=12.9, 9.0 Hz, 1H), 1.99-1.89 (m, 7H), 1.86 (d, J=11.8 Hz, 2H), 1.55-1.51 (m, 2H), 1.06 (d, J=6.4 Hz, 4H), 0.78 (s, 3H). [M+H]⁺=984.3.

### Example 742: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3-ethyl-6,7-difluoro-2-oxo-2,3-dihydro-1H-benzo[d]imidazol-1-yl)piperidine-2,6-dione

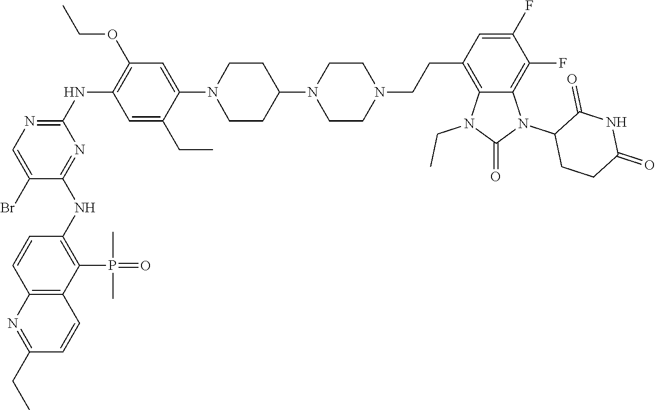 [SMILES: CCOc1cc(N2CCC(N3CCN(CCc4cc(F)c(F)c5c4n(CC)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 318. ¹H NMR (500 MHz, DMSO) δ 11.72 (s, 1H), 11.16 (s, 1H), 8.60 (d, J=8.8 Hz, 1H), 8.22 (s, 2H), 7.91 (s, 1H), 7.88 (d, J=9.2 Hz, 1H), 7.45 (d, J=8.9 Hz, 1H), 7.38 (s, 1H), 7.09 (dd, J=12.5, 7.8 Hz, 1H), 6.71 (s, 1H), 5.57 (dd, J=12.9, 5.2 Hz, 1H), 4.08-3.94 (m, 4H), 2.94 (dt, J=14.4, 7.1 Hz, 8H), 2.71-2.60 (m, 4H), 2.59-2.52 (m, 8H), 2.25 (s, 4H), 2.13 (s, 1H), 1.98 (d, J=13.3 Hz, 6H), 1.83 (d, J=11.2 Hz, 2H), 1.53-1.47 (m, 2H), 1.32 (t, J=7.6 Hz, 3H), 1.26 (t, J=6.9 Hz, 6H), 0.71 (s, 3H). [M+H]⁺=1070.4

### Example 743: 3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-5-ethoxy-2-ethylphenyl)piperidin-4-yl)piperazin-1-yl)ethyl)-3-ethyl-6,7-difluoro-2-oxo-2,3-dihydro-1H-benzo[d]imidazol-1-yl)piperidine-2,6-dione

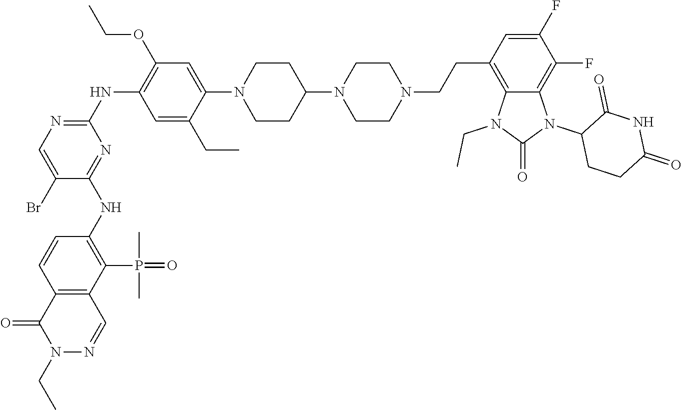 [SMILES: CCOc1cc(N2CCC(N3CCN(CCc4cc(F)c(F)c5c4n(CC)c(=O)n5C4CCC(=O)NC4=O)CC3)CC2)c(CC)cc1Nc1ncc(Br)c(Nc2ccc3c(=O)n(CC)ncc3c2P(C)(C)=O)n1]

The title compound was prepared in a procedure similar to that in Example 318. ¹H NMR (500 MHz, DMSO) δ 12.11 (s, 1H), 11.16 (s, 1H), 8.64 (s, 1H), 8.52 (s, 1H), 8.26 (s, 1H), 8.21-8.15 (m, 1H), 8.10 (s, 1H), 7.33 (s, 1H), 7.09 (dd, J=12.6, 7.9 Hz, 1H), 6.73 (s, 1H), 5.57 (dd, J=12.5, 5.2 Hz, 1H), 4.17 (q, J=7.1 Hz, 3H), 4.04-4.00 (m, 5H), 2.97 (d, J=7.9 Hz, 6H), 2.73-2.54 (m, 4H), 2.29 (d, J=7.8 Hz, 6H), 2.13 (s, 1H), 2.03 (d, J=13.3 Hz, 7H), 1.85 (d, J=10.9 Hz, 2H), 1.63-1.49 (m, 3H), 1.32 (s, 4H), 1.25 (s, 7H), 0.88 (s, 3H). [M+H]⁺=1087.4

### Example 744: (R)-3-(4-(2-(4-(1-(4-((5-chloro-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

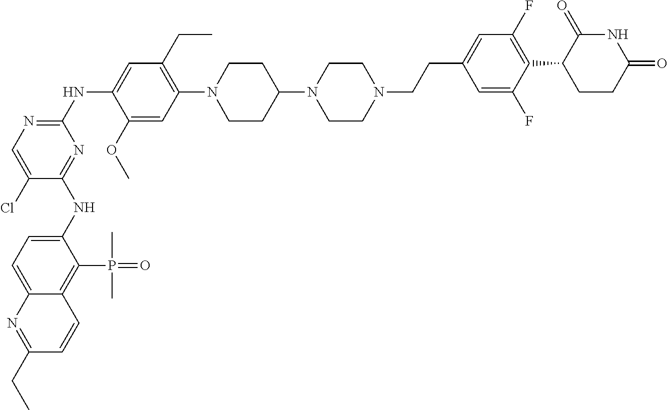 [SMILES: CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC)ncc3Cl)ccc2n1]

The title compound was prepared in a procedure similar to that in Example 486. ¹H NMR (500 MHz, DMSO) δ 12.04 (s, 1H), 10.96 (s, 1H), 8.56 (s, 1H), 8.45 (s, 1H), 8.14 (d, J=6.9 Hz, 1H), 8.03 (s, 1H), 7.89 (d, J=9.0 Hz, 1H), 7.45 (d, J=9.0 Hz, 2H), 7.06 (d, J=10.1 Hz, 2H), 6.76 (s, 1H), 4.21 (d, J=8.5 Hz, 1H), 3.77 (s, 3H), 3.54 (s, 1H), 3.01 (s, 4H), 2.93 (q, J=7.5 Hz, 2H), 2.86-2.76 (m, 2H), 2.74-2.67 (m, 4H), 2.55 (s, 2H), 2.52 (s, 6H), 2.45-2.30 (m, 3H), 2.21-2.10 (m, 2H), 2.00 (d, J=13.3 Hz, 7H), 1.81 (s, 1H), 1.58 (s, 1H), 1.32 (t, J=7.6 Hz, 3H), 0.82 (s, 3H). [M+H]⁺=928.4.

### Example 745: (R)-3-(4-(2-(1′-(4-((5-bromo-4-((5-(dimethylphosphoryl)-2-ethylquinolin-6-yl)amino)pyrimidin-2-yl)amino)-2-ethyl-5-methoxyphenyl)-[4,4′-bipiperidin]-1-yl)ethyl)-2,6-difluorophenyl)piperidine-2,6-dione

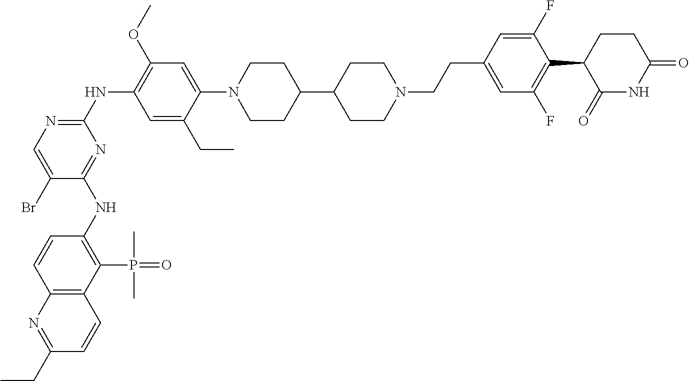 [SMILES: CCc1ccc2c(P(C)(C)=O)c(Nc3nc(Nc4cc(CC)c(N5CCC(C6CCN(CCc7cc(F)c([C@H]8CCC(=O)NC8=O)c(F)c7)CC6)CC5)cc4OC)ncc3Br)ccc2n1]

The title compound was prepared in a procedure similar to that in Example 486. ¹H NMR (500 MHz, DMSO) δ 11.79 (s, 1H), 10.96 (s, 1H), 8.56 (d, J=8.9 Hz, 1H), 8.27 (s, 1H), 8.21 (s, 1H), 8.01 (s, 1H), 7.87 (d, J=9.3 Hz, 1H), 7.44 (d, J=8.9 Hz, 1H), 7.33 (s, 1H), 7.07 (d, J=10.0 Hz, 2H), 6.74 (s, 1H), 4.22 (dd, J=12.6, 4.9 Hz, 1H), 3.76 (s, 3H), 2.93 (s, 7H), 2.80 (dd, J=13.2, 5.0 Hz, 1H), 2.63 (t, J=10.9 Hz, 3H), 2.54-2.50 (m, 6H), 2.29 (d, J=6.7 Hz, 2H), 2.13-2.09 (m, 1H), 1.98 (d, J=13.3 Hz, 7H), 1.87-1.73 (m, 4H), 1.32 (t, J=7.6 Hz, 7H), 1.23 (s, 1H), 0.77 (s, 3H). [M+H]⁺=971.3.

### Example 746: (R)-3-(4-((R)-4-(4-(1-(4-((5-bromo-4-((5-(dimethylphosphoryl)-2-ethyl-8-fluoroquinolin-6-yl)amino)pyrimidin-2-yl)amino)-5-ethoxy-2-ethylphenyl)piperidin-4-yl)piperazine-1-carbonyl)-3,3-dimethylpyrrolidin-1-yl)-2,6-difluorophenyl)piperidine-2,6-dione

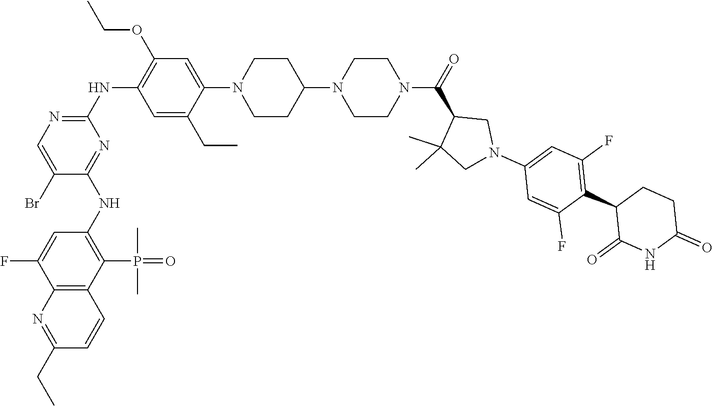 [SMILES: CCOc1cc(N2CCC(N3CCN(C(=O)[C@H]4CN(c5cc(F)c([C@H]6CCC(=O)NC6=O)c(F)c5)CC4(C)C)CC3)CC2)c(CC)cc1Nc1ncc(Br)c(Nc2cc(F)c3nc(CC)ccc3c2P(C)(C)=O)n1]

The title compound was prepared in a procedure similar to that in Example 488. ¹H NMR (500 MHz, DMSO) δ 12.10 (s, 1H), 10.84 (s, 1H), 8.57 (d, J=7.0 Hz, 1H), 8.32-8.27 (m, 2H), 8.05 (s, 1H), 7.54 (d, J=9.0 Hz, 1H), 7.39 (s, 1H), 6.71 (s, 1H), 6.15 (d, J=12.2 Hz, 2H), 4.01 (d, J=7.0 Hz, 3H), 3.70-3.57 (m, 8H), 3.15-3.05 (m, 3H), 2.95 (d, J=7.6 Hz, 4H), 2.84-2.73 (m, 2H), 2.68-2.59 (m, 4H), 2.40-2.23 (m, 3H), 2.00 (d, J=13.3 Hz, 8H), 1.83 (d, J=10.6 Hz, 2H), 1.55 (d, J=10.8 Hz, 2H), 1.32 (t, J=7.6 Hz, 3H), 1.27 (d, J=6.9 Hz, 3H), 1.18 (s, 3H), 0.99 (s, 3H), 0.74 (s, 3H). [M+H]⁺=1101.4.

### Example 747: (R)-3-(4-((R)-4-(4-(1-(4-((5-bromo-4-((5-(dimethylphosphoryl)-2-methylquinolin-6-yl)amino)pyrimidin-2-yl)amino)-5-ethoxy-2-ethylphenyl)piperidin-4-yl)piperazine-1-carbonyl)-3,3-dimethylpyrrolidin-1-yl)-2,6-difluorophenyl)piperidine-2,6-dione

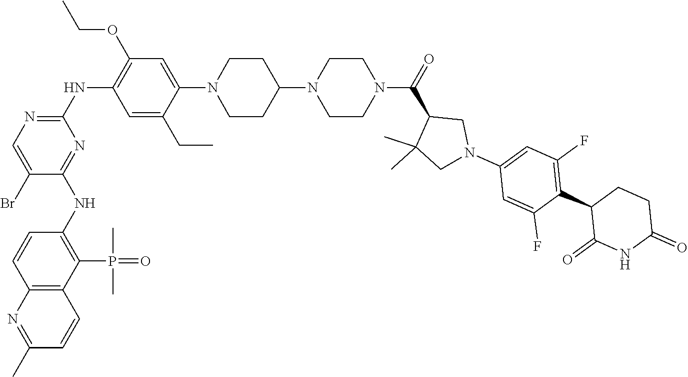 [SMILES: CCOc1cc(N2CCC(N3CCN(C(=O)[C@H]4CN(c5cc(F)c([C@H]6CCC(=O)NC6=O)c(F)c5)CC4(C)C)CC3)CC2)c(CC)cc1Nc1ncc(Br)c(Nc2ccc3nc(C)ccc3c2P(C)(C)=O)n1]

The title compound was prepared in a procedure similar to that in Example 488. ¹H NMR (500 MHz, DMSO) δ 11.62 (s, 1H), 10.77 (s, 1H), 8.53 (d, J=8.9 Hz, 1H), 8.18 (d, J=21.2 Hz, 2H), 7.95-7.77 (m, 2H), 7.36 (d, J=8.7 Hz, 2H), 6.63 (s, 1H), 6.09 (d, J=12.2 Hz, 2H), 3.93 (q, J=6.9 Hz, 3H), 3.60-3.45 (m, 8H), 3.02 (dd, J=18.7, 9.2 Hz, 2H), 2.86 (d, J=10.7 Hz, 2H), 2.76-2.67 (m, 2H), 2.58 (s, 6H), 2.29 (s, 1H), 2.19 (d, J=5.8 Hz, 2H), 2.06-1.85 (m, 9H), 1.75 (d, J=10.6 Hz, 2H), 1.49 (d, J=10.7 Hz, 2H), 1.19 (t, J=6.9 Hz, 4H), 1.10 (d, J=10.9 Hz, 3H), 0.92 (s, 3H), 0.64 (s, 3H). [M+H]⁺=1069.4.

### Example 748: (R)-3-(4-(2-(4-(1-(4-((5-bromo-4-((5-(dimethylphosphoryl)-2-ethylquinazolin-6-yl)amino)pyrimidin-2-yl)amino)-5-methoxy-2-methylphenyl)piperidin-4-yl)piperazin-1-yl)ethyl)-2,6-difluorophenyl)piperidine-2,6-dione

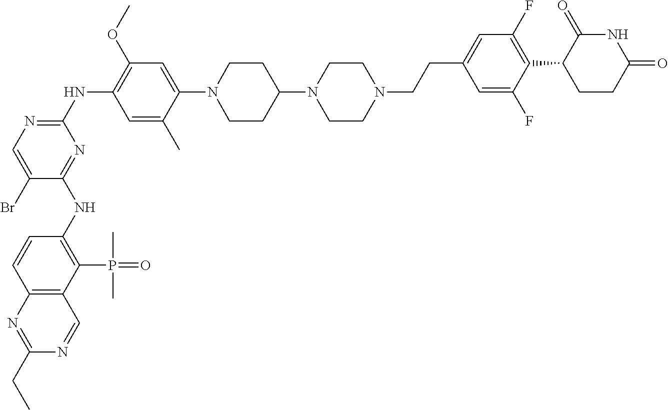 [SMILES: CCc1ncc2c(P(C)(C)=O)c(Nc3nc(Nc4cc(C)c(N5CCC(N6CCN(CCc7cc(F)c([C@H]8CCC(=O)NC8=O)c(F)c7)CC6)CC5)cc4OC)ncc3Br)ccc2n1]

The title compound was prepared in a procedure similar to that in Example 460. ¹H NMR (400 MHz, DMSO) δ 11.74 (s, 1H), 10.95 (s, 1H), 9.82 (s, 1H), 8.48 (s, 1H), 8.22 (s, 1H), 8.05 (s, 1H), 7.84 (s, 1H), 7.23 (s, 1H), 7.02 (d, J=10.0 Hz, 2H), 6.67 (s, 1H), 4.20 (dd, J=12.6, 5.0 Hz, 1H), 3.75 (s, 3H), 3.01 (s, 4H), 2.85-2.82 (m, 1H), 2.77-2.74 (m, 3H), 2.62-2.57 (m, 3H), 2.55-2.53 (m, 6H), 2.48-2.45 (m, 2H), 2.27 (s, 1H), 2.19-2.10 (m, 2H), 2.03-1.98 (m, 6H), 1.89-1.82 (m, 6H), 1.54 (d, J=9.7 Hz, 2H), 1.36 (d, J=6.8 Hz, 3H). [M+H]⁺=959.0.

### Example 749: (R)-3-(4-(2-(4-(1-(4-((4-((5-(dimethylphosphoryl)-2-ethylquinazolin-6-yl)amino)pyrimidin-2-yl)amino)-5-ethoxy-2-ethylphenyl)piperidin-4-yl)piperazin-1-yl)ethyl)-2,6-difluorophenyl)piperidine-2,6-dione

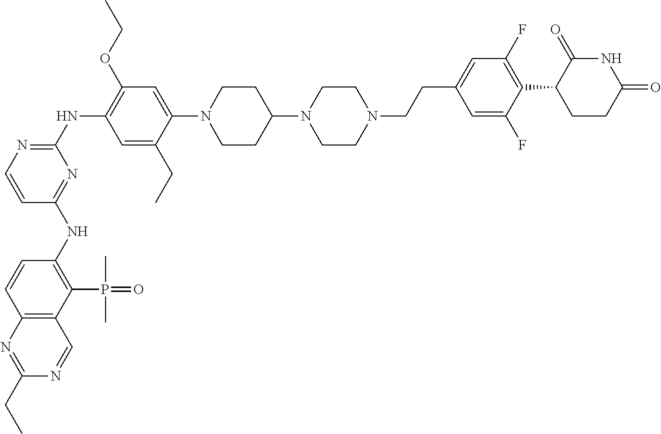 [SMILES: CCOc1cc(N2CCC(N3CCN(CCc4cc(F)c([C@H]5CCC(=O)NC5=O)c(F)c4)CC3)CC2)c(CC)cc1Nc1nccc(Nc2ccc3nc(CC)ncc3c2P(C)(C)=O)n1]

The title compound was prepared in a procedure similar to that in Example 460. ¹H NMR (400 MHz, DMSO) δ 11.01 (s, 1H), 10.95 (s, 1H), 10.18 (s, 1H), 8.57 (s, 1H), 8.07 (d, J=5.6 Hz, 1H), 7.95 (d, J=9.1 Hz, 1H), 7.69 (s, 1H), 7.57 (s, 1H), 7.04 (d, J=10.1 Hz, 2H), 6.71 (s, 1H), 6.18 (d, J=5.6 Hz, 1H), 4.20 (dd, J=12.6, 4.9 Hz, 1H), 4.05-4.01 (m, 2H), 3.07-3.03 (m, 2H), 2.92 (d, J=10.2 Hz, 2H), 2.83-2.79 (m, 1H), 2.78-2.75 (m, 3H), 2.66-2.59 (m, 5H), 2.55-2.52 (m, 7H), 2.24-2.21 (m, 2H), 2.13 (dd, J=13.4, 3.7 Hz, 1H), 1.99 (d, J=13.4 Hz, 8H), 1.89 (s, 2H), 1.57 (s, 2H), 1.37 (t, J=7.6 Hz, 3H), 1.30 (t, J=6.9 Hz, 3H), 0.72 (s, 3H). [M+H]⁺=909.2.

### Example 750: (R)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

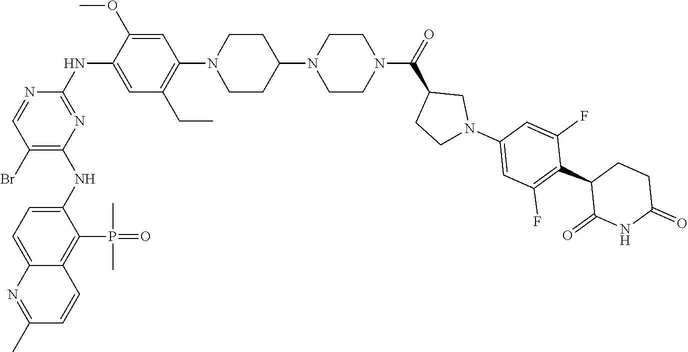 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

The title compound was prepared in a procedure similar to that in Example 488. ¹H NMR (500 MHz, DMSO) δ 11.76 (s, 1H), 10.84 (s, 1H), 8.56 (d, J=8.9 Hz, 1H), 8.31 (d, J=5.4 Hz, 1H), 8.21 (s, 1H), 7.98 (s, 1H), 7.87 (d, J=9.2 Hz, 1H), 7.42 (d, J=8.9 Hz, 1H), 7.38 (s, 1H), 6.74 (s, 1H), 6.23 (d, J=12.2 Hz, 2H), 4.02 (dd, J=12.5, 4.9 Hz, 1H), 3.76 (s, 3H), 3.59-3.42 (m, 6H), 3.36-3.32 (m, 1H), 3.31-3.22 (m, 2H), 2.95 (d, J=10.6 Hz, 2H), 2.83-2.73 (m, 1H), 2.71-2.62 (m, 5H), 2.61-2.51 (m, 3H), 2.48-2.44 (m, 2H), 2.42-2.35 (m, 1H), 2.35-2.25 (m, 2H), 2.20-2.04 (m, 3H), 2.02-1.91 (m, 7H), 1.84 (d, J=10.2 Hz, 2H), 1.65-1.51 (m, 2H), 0.77 (s, 3H). [M+H]⁺=1027.7.

### Example 751: (R)-3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

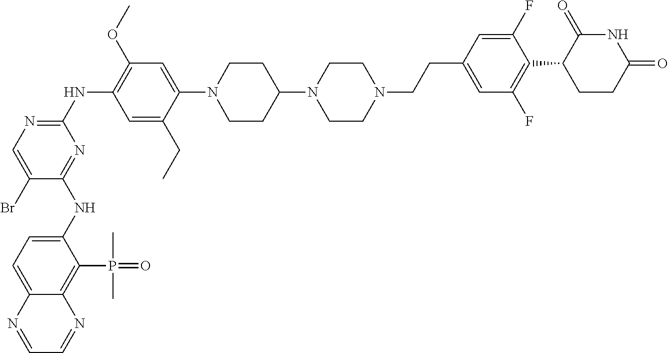 [SMILES: CCc1cc(Nc2ncc(Br)c(Nc3ccc4nccnc4c3P(C)(C)=O)n2)c(OC)cc1N1CCC(N2CCN(CCc3cc(F)c([C@H]4CCC(=O)NC4=O)c(F)c3)CC2)CC1]

The title compound was prepared in a procedure similar to that in Example 486.

¹H NMR (500 MHz, DMSO) δ 12.65 (s, 1H), 10.96 (s, 1H), 8.86 (dt, J=22.8, 11.4 Hz, 3H), 8.28 (d, J=9.1 Hz, 2H), 7.90 (d, J=8.9 Hz, 1H), 7.37 (s, 1H), 7.03 (d, J=10.0 Hz, 2H), 6.81 (s, 1H), 4.20 (dd, J=12.8, 5.0 Hz, 1H), 3.77 (s, 3H), 3.01 (d, J=11.5 Hz, 2H), 2.76 (m, 6H), 2.54 (d, J=1.8 Hz, 6H), 2.48 (s, 5H), 2.36 (s, 2H), 2.13 (d, J=9.6 Hz, 1H), 2.02 (d, J=14.4 Hz, 7H), 1.87 (d, J=11.4 Hz, 2H), 1.58 (d, J=8.8 Hz, 2H), 0.93 (t, J=7.2 Hz, 3H); [M+H]⁺=945.4.

### Example 752: 3-(4-(2-(4-(1-(4-((5-bromo-4-((5-(dimethylphosphoryl)-2-ethylquinolin-6-yl)amino)pyrimidin-2-yl)amino)-5-ethoxy-2-ethylphenyl)piperidin-4-yl)piperazin-1-yl)ethyl)-3-methyl-2-oxo-2,3-dihydro-1H-benzo[d]imidazol-1-yl)piperidine-2,6-dione

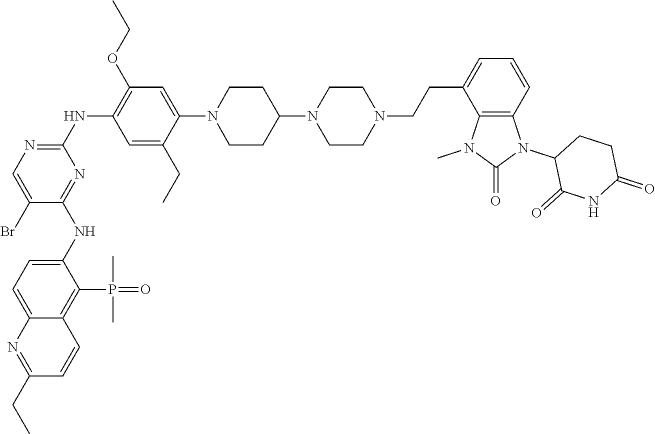 [SMILES: CCOc1cc(N2CCC(N3CCN(CCc4cccc5c4n(C)c(=O)n5C4CCC(=O)NC4=O)CC3)CC2)c(CC)cc1Nc1ncc(Br)c(Nc2ccc3nc(CC)ccc3c2P(C)(C)=O)n1]

The title compound was prepared in a procedure similar to that in Example 318. ¹H NMR (500 MHz, DMSO) δ 11.73 (s, 1H), 11.10 (s, 1H), 8.60 (d, J=8.9 Hz, 1H), 8.29-8.17 (m, 2H), 7.92 (s, 1H), 7.88 (d, J=9.3 Hz, 1H), 7.45 (d, J=8.9 Hz, 1H), 7.39 (s, 1H), 7.05-6.95 (m, 2H), 6.90 (dd, J=6.3, 2.5 Hz, 1H), 6.71 (s, 1H), 5.37 (dd, J=12.7, 5.3 Hz, 1H), 4.00 (q, J=7.0 Hz, 2H), 3.58 (s, 3H), 3.22-3.20 (m, 2H), 3.09-3.03 (m, 2H), 2.96-2.85 (m, 5H), 2.76-2.51 (m, 12H), 2.27-2.23 (m, 3H), 2.02-1.93 (m, 7H), 1.83 (d, J=10.7 Hz, 2H), 1.53-1.49 (m, 2H), 1.32 (t, J=7.6 Hz, 3H), 1.26 (t, J=6.9 Hz, 3H), 0.71 (s, 3H). [M+H]⁺=1020.5.

### Example 753: (R)-3-(4-(2-(4-(1-(4-((5-bromo-4-((5-(dimethylphosphoryl)-2-ethyl-1-oxo-1,2-dihydrophthalazin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

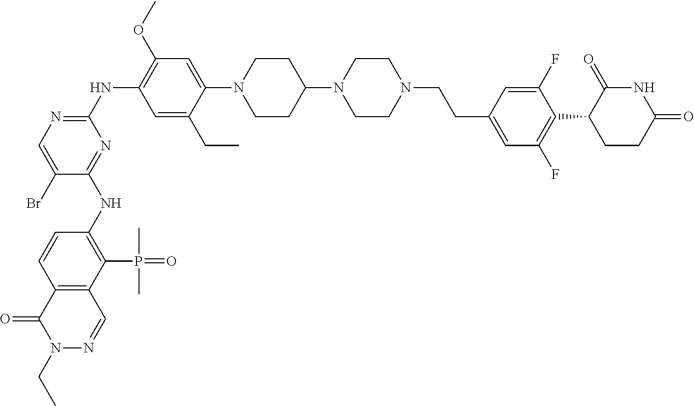 [SMILES: CCc1cc(Nc2ncc(Br)c(Nc3ccc4c(=O)n(CC)ncc4c3P(C)(C)=O)n2)c(OC)cc1N1CCC(N2CCN(CCc3cc(F)c([C@H]4CCC(=O)NC4=O)c(F)c3)CC2)CC1]

The title compound was prepared in a procedure similar to that in Example 486. ¹H NMR (500 MHz, DMSO) δ 12.17 (s, 1H), 10.95 (s, 1H), 8.62 (s, 1H), 8.54 (s, 1H), 8.24 (s, 1H), 8.18 (d, J=6.0 Hz, 2H), 7.31 (s, 1H), 7.04 (s, 1H), 7.02 (s, 1H), 6.76 (s, 1H), 4.22-4.15 (m, 3H), 3.75 (s, 3H), 2.98 (d, J=10.6 Hz, 2H), 2.85-2.74 (m, 4H), 2.67 (t, J=11.1 Hz, 3H), 2.55-2.53 (m, 7H), 2.48-2.38 (m, 5H), 2.32 (t, J=11.1 Hz, 1H), 2.17-2.10 (m, 1H), 2.03 (d, J=13.4 Hz, 6H), 1.85 (d, J=10.9 Hz, 2H), 1.56 (d, J=11.4 Hz, 2H), 1.31 (t, J=7.2 Hz, 3H), 0.91 (s, 3H). [M+H]⁺=989.7.

### Example 754: (R)-3-(4-((R)-4-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)-3,3-dimethylpyrrolidin-1-yl)-2,6-difluorophenyl)piperidine-2,6-dione

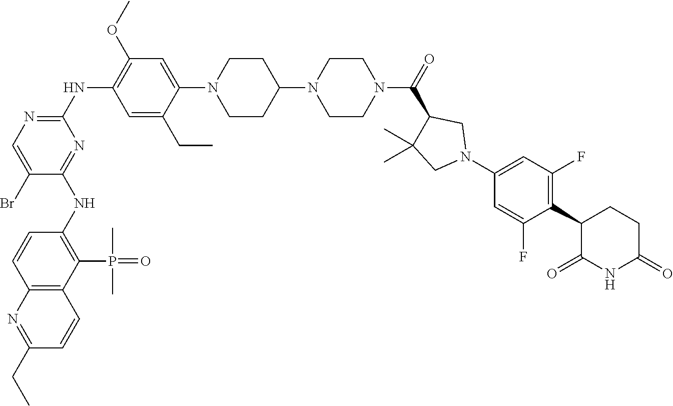 [SMILES: CCc1ccc2c(P(C)(C)=O)c(Nc3nc(Nc4cc(CC)c(N5CCC(N6CCN(C(=O)[C@H]7CN(c8cc(F)c([C@H]9CCC(=O)NC9=O)c(F)c8)CC7(C)C)CC6)CC5)cc4OC)ncc3Br)ccc2n1]

The title compound was prepared in a procedure similar to that in Example 486. ¹H NMR (500 MHz, DMSO) δ 11.79 (s, 1H), 10.84 (s, 1H), 8.56 (d, J=8.7 Hz, 1H), 8.27 (s, 1H), 8.21 (s, 1H), 8.01 (s, 1H), 7.87 (d, J=9.4 Hz, 1H), 7.44 (d, J=8.9 Hz, 1H), 7.33 (s, 1H), 6.75 (s, 1H), 6.15 (d, J=12.2 Hz, 2H), 4.01 (dd, J=12.4, 4.9 Hz, 1H), 3.76 (s, 3H), 3.60 (s, 2H), 3.52 (d, J=13.0 Hz, 2H), 3.45 (d, J=5.3 Hz, 2H), 3.42-3.35 (m, 4H), 3.05-3.15 (m, 2H), 3.00-2.89 (m, 4H), 2.83-2.73 (m, 1H), 2.68 (t, J=11.3 Hz, 2H), 2.50-2.55 (m, 2H), 2.37 (s, 1H), 2.29 (d, J=6.6 Hz, 2H), 2.08-2.12 (m, 1H), 1.98 (d, J=13.3 Hz, 7H), 1.84 (d, J=10.5 Hz, 2H), 1.57 (d, J=11.2 Hz, 2H), 1.32 (s, 3H), 1.19 (s, 3H), 0.99 (s, 3H), 0.77 (s, 3H). [M+H]⁺=1069.4.

### Example 755: (R)-3-(4-(3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)azetidin-1-yl)-2,6-difluorophenyl)piperidine-2,6-dione

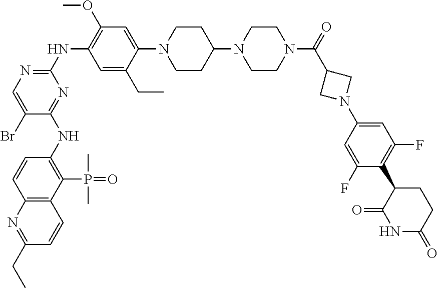 [SMILES: CCc1ccc2c(P(C)(C)=O)c(Nc3nc(Nc4cc(CC)c(N5CCC(N6CCN(C(=O)C7CN(c8cc(F)c([C@H]9CCC(=O)NC9=O)c(F)c8)C7)CC6)CC5)cc4OC)ncc3Br)ccc2n1]

The title compound was prepared in a procedure similar to that in Example 488. ¹H NMR (500 MHz, DMSO) δ 11.80 (s, 1H), 10.86 (s, 1H), 8.56 (d, J=8.9 Hz, 1H), 8.28 (d, J=7.3 Hz, 1H), 8.21 (s, 1H), 8.00 (s, 1H), 7.87 (d, J=9.2 Hz, 1H), 7.44 (d, J=8.9 Hz, 1H), 7.34 (s, 1H), 6.75 (s, 1H), 6.17 (d, J=11.1 Hz, 2H), 4.07-4.00 (m, 3H), 3.92 (t, J=6.0 Hz, 2H), 3.87-3.79 (m, 1H), 3.76 (s, 3H), 3.49 (s, 2H), 3.32 (s, 3H), 3.00-2.88 (m, 4H), 2.84-2.73 (m, 1H), 2.72-2.63 (m, 2H), 2.51-2.44 (m, 4H), 2.42-2.34 (m, 1H), 2.34-2.25 (m, 2H), 2.13-2.03 (m, 1H), 2.02-1.92 (m, 7H), 1.82 (d, J=10.5 Hz, 2H), 1.63-1.51 (m, 2H), 1.32 (t, J=7.6 Hz, 3H), 0.77 (s, 3H). [M+H]⁺=1027.7.

### Example 756: 3-(4′-(2-(4-(1-(4-((5-bromo-4-((5-(dimethylphosphoryl)-2-methyl-1-oxo-1,2-dihydrophthalazin-6-yl)amino)pyrimidin-2-yl)amino)-2-ethyl-5-methoxyphenyl)piperidin-4-yl)piperazin-1-yl)ethyl)-2′-oxospiro[cyclopropane-1,3′-indolin]-1′-yl)piperidine-2,6-dione

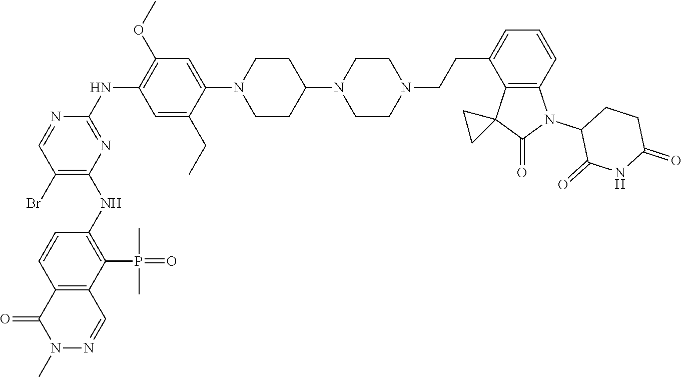 [SMILES: CCc1cc(Nc2ncc(Br)c(Nc3ccc4c(=O)n(C)ncc4c3P(C)(C)=O)n2)c(OC)cc1N1CCC(N2CCN(CCc3cccc4c3C3(CC3)C(=O)N4C3CCC(=O)NC3=O)CC2)CC1]

The title compound was prepared in a procedure similar to that in Example 486. ¹H NMR (500 MHz, DMSO) δ 12.21 (s, 1H), 11.08 (s, 1H), 8.63-8.51 (m, 2H), 8.23-8.16 (m, 3H), 7.34 (s, 1H), 7.15 (t, J=7.8 Hz, 1H), 6.88 (d, J=7.7 Hz, 2H), 6.76 (s, 1H), 5.31 (s, 1H), 3.75 (s, 3H), 3.72 (s, 3H), 2.99 (d, J=10.3 Hz, 2H), 2.88 (t, J=12.5 Hz, 1H), 2.71-2.60 (m, 4H), 2.57-2.54 (m, 6H), 2.49-2.37 (m, 9H), 2.32 (t, J=11.0 Hz, 1H), 2.03 (d, J=13.4 Hz, 6H), 1.95 (d, J=3.4 Hz, 2H), 1.85 (d, J=10.8 Hz, 2H), 1.57 (d, J=11.4 Hz, 2H), 1.48 (d, J=3.7 Hz, 2H), 0.93 (s, 3H). [M+H]⁺=1020.7.

### tert-butyl-4-(1-(4-amino-5-ethoxy-2-methylphenyl)piperidin-4-yl)piperazine-1-carboxylate

### Step 1: tert-butyl 4-(1-(5-ethoxy-2-methyl-4-nitrophenyl)piperidin-4-yl)piperazine-1-carboxylate

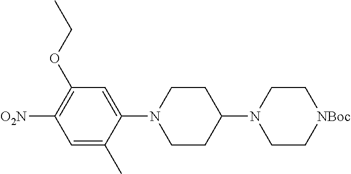 [SMILES: CCOc1cc(N2CCC(N3CCN(C(=O)OC(C)(C)C)CC3)CC2)c(C)cc1[N+](=O)[O-]]

The titled compound (740 mg, 88%) was prepared in a manner similar to that in Example 460 step 8 from 1-ethoxy-5-fluoro-4-methyl-2-nitrobenzene and tert-butyl 4-(piperidin-4-yl)piperazine-1-carboxylate. [M+H]⁺=449.3.

### Step 2: tert-butyl 4-(1-(4-amino-5-ethoxy-2-methylphenyl)piperidin-4-yl)piperazine-1-carboxylate

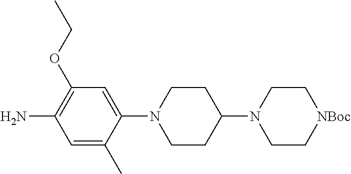 [SMILES: CCOc1cc(N2CCC(N3CCN(C(=O)OC(C)(C)C)CC3)CC2)c(C)cc1N]

The titled compound (520 mg, 78%) was prepared in a manner similar to that in Example 460 step 9 from tert-butyl 4-(1-(5-ethoxy-2-methyl-4-nitrophenyl)piperidin-4-yl)piperazine-1-carboxylate and Pd/C. [M+H]⁺=419.3.

Cell Degradation

Cell Line Generation

H1975-clone #36(L858R/T790M). H1975 cell heterozygously (from ATCC) contains alleles of WT and L858R/T790M EGFR. H1975-clone #36(L858R/T790M) was generated by gene knockout, in which the EGFR targeting sgRNA was designed to specifically target WT EGFR but preserve the L858R/T790M copy. Followed by the gene knockout, the edited H1975 cells were seeded in 96 well plates at the concentration of 1 cell/well, cultured for about 2 weeks to allow single clones formation. The formed clones were screened by DNA sequencing for the desired edition. H1975-clone #36 was confirmed as a homozygous L858R/T790M EGFR clone.

H1975-clone #28(Del19/T790M/C797S) and H1975-clone #23(Del19/C797S). EGFR-Del19/T790M/C797S and EGFR-Del19/C797S were stably expressed in H1975 cell lines by lentivirus-mediated over-expression, respectively. The EGFR over-expressed cells then underwent gene knockout, in which the EGFR targeting sgRNA was designed to only target the endogenous EGFR copies and preserve the exogenous EGFR copies. Followed by the gene knockout, the edited H1975 cells were seeded in 96 well plates at the concentration of 1 cell/well, cultured for about 2 weeks to allow single clones formation. The formed clones were screened by DNA sequencing and whole exon sequencing analysis for the desired edition. H1975-clone #28 and H1975-clone #23 were finally confirmed as homozygous Del19/T790M/C797S EGFR and Del19/C797S EGFR clones, respectively.

Cell Treatment

1a). BaF3 WT, BaF3-LTC(L858R/T790M/C797S), BaF3-DTC(Del19/T790M/C797S) and BaF3 LC(L858R/C797S) cells are seeded at 50000 cells/well (WT) or 20000 cells/well (LTC, DTC&LC) in cell culture medium [RPMI1640(Gibco, phenol red free, Cat #11835-030), 10% heat-inactive FBS, 1% PS(Gibco, Cat #10378)] in Corning 96 well plate (Cat #3799).

1b). On day 1, HCC827, H1975-clone #36(L858R/T790M, homozygous), H1975-clone #28(Del19/T790M/C797S) and H1975-clone #23(Del19/C797S) cells are seeded at 20000 cells/well, 30000cells/well, 10000 cells/well or 5000 cells/well correspondingly in cell culture medium [RPMI1640(Gibco, Cat #72400-047), 10% heat-inactive FBS, 1% PS(Gibco, Cat #10378)] in Corning 96 well plate (Cat #3599).

BaF3 WT(wild type), BaF3-LTC(L858R/T790M/C797S) and BaF3-DTC(Del19/T790M/C797S) cells are treated with compounds diluted in 0.2% DMSO cell culture medium and incubate for 16h, 37° C., 5% CO₂, H1975-#36, H1975-#28, H1975-#23 and HCC827 cells are treated with compounds diluted in 0.2% DMSO cell culture medium on day 2, incubate for 16h, 37° C., 5% CO₂.the final concentriation of compounds in all assay is start with 10 uM, 5-fold dilution, total 8 doses were included.

HTRF Assay

After 16h treatment, add HTRF lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation: Inhibition percentage of Compound=100-100×(Signal-low control)/(High control-low control), wherein signal=each test compound group

Low control=only lysis buffer without cells, indicating that EGFR is completely degraded;

High control=Cell group with added DMSO and without compound, indicating microplate readings without EGFR degradation;

Dmax is the maximum percentage of inhibition (degradation).

The IC₅₀ (DC₅₀) value of a compound can be obtained by fitting the following equation

Y=Bottom+(TOP−Bottom)/(1+((IC₅₀/X)\hat{ }hillslope))

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation (BaF3) result for Example 1 to Example 723

| | WT | | DTC | | LTC | |
|---|---|---|---|---|---|---|
| Example | DC₅₀(nM) | Dmax (%) | DC₅₀(nM) | Dmax (%) | DC₅₀(nM) | Dmax (%) |
| 1 | >10000.0 | 49.21 | 6.87 | 92.16 | 18.23 | 89.29 |
| 5 | >10000.0 | 47.9 | 4.96 | 96.75 | 13.08 | 90.42 |
| 6 | | | 9.2 | 81.2 | 102.1 | 82.23 |
| 7 | >10000.0 | 41.89 | 18.25 | 95.15 | 36.41 | 90.49 |
| 10 | | | 15.45 | 85.51 | 83.3 | 88.69 |
| 11 | | | 3.74 | 95.55 | 8.82 | 91.88 |
| 23 | >10000.0 | 14.64 | 13.33 | 95.73 | 25.62 | 88.97 |
| 34 | >10000.0 | 26.24 | 15.07 | 82.04 | 21.57 | 81.25 |
| 48 | 2916.2 | 52.61 | 7.6 | 92.6 | 24.32 | 90.42 |
| 56 | >10000.0 | 16.87 | 9.52 | 82.17 | 31.44 | 79.46 |
| 60 | 8860.1 | 51.6 | 42.22 | 94.93 | 89.02 | 91.19 |
| 61 | >10000.0 | 22.24 | 6.05 | 96.02 | 20.11 | 90.41 |
| 63 | >10000.0 | 3.27 | 281.4 | 52.42 | >2000.0 | 38.45 |
| 64 | | | 4.85 | 90.07 | 1.72 | 85.39 |
| 65 | >10000.0 | 15.44 | 8.05 | 80.45 | 14.19 | 75.48 |
| 66 | | | 4.19 | 92.43 | 10.95 | 86.27 |
| 67 | | | 4.71 | 94.22 | 13.29 | 87.89 |
| 70 | >10000.0 | 26.11 | 8.49 | 91.36 | 12.18 | 95.03 |
| 71 | >10000.0 | 31.43 | 7.52 | 81.19 | 11.48 | 80.04 |
| 101 | >10000.0 | 30.08 | 8.86 | 96.43 | 9.53 | 94.88 |
| 113 | >10000.0 | 29.05 | 15.14 | 99.16 | 77.21 | 90.97 |
| 123 | >10000.0 | 21.57 | 272.8 | 83.94 | >10000.0 | 21.2 |
| 128 | >10000.0 | 15.32 | 53.69 | 99.67 | 167.6 | 86.15 |
| 159 | | | 9.34 | 87.25 | 16.12 | 88.35 |
| 160 | | | 6.95 | 94.14 | 18.12 | 92.93 |
| 165 | | | 18.12 | 82.21 | 44.03 | 84.24 |
| 166 | | | 13.49 | 85.99 | 49.27 | 80.08 |
| 168 | | | 22.36 | 88.5 | 47.05 | 86.39 |
| 178 | | | 44.36 | 81.18 | 71.84 | 83.53 |
| 199 | | | 9.03 | 84.49 | 22.11 | 86.6 |
| 200 | | | 2.8 | 93.3 | 11.05 | 88.58 |
| 201 | | | 1.72 | 95.68 | 6.8 | 91.95 |
| 202 | | | 7.6 | 93.39 | 18.5 | 87.75 |
| 203 | >10000.0 | 35.28 | 13.5 | 92.4 | 17.01 | 82.14 |
| 204 | | | 15.55 | 91.45 | 30.95 | 85.71 |
| 205 | | | 6.63 | 90.39 | 32.52 | 87.69 |
| 206 | | | 9.26 | 94.22 | 20.89 | 92.1 |
| 207 | | | 6.6 | 90.41 | 13.23 | 91.28 |
| 208 | | | 1.47 | 97.62 | 4.13 | 93.88 |
| 209 | | | 2.1 | 96.2 | 3.92 | 95.45 |
| 210 | | | 0.781 | 92.69 | 1.24 | 90.47 |
| 211 | | | 2.72 | 94.88 | 4.75 | 91.44 |
| 212 | | | 16.59 | 94.28 | 81.22 | 78.76 |
| 213 | | | 4.71 | 94.05 | 21.1 | 88.64 |
| 214 | | | 13.69 | 85.16 | 54.86 | 75.01 |
| 215 | | | 4.91 | 96.67 | 20.48 | 91.42 |
| 216 | | | 5.56 | 93.59 | 23.29 | 87.7 |
| 217 | | | 29.7 | 90.75 | 86.76 | 89.63 |
| 218 | | | 2.9 | 97.83 | 9.19 | 87.27 |
| 219 | | | 1.94 | 93 | 4.8 | 87.51 |
| 220 | | | 18.18 | 86 | 43.15 | 88.79 |
| 221 | | | 15.68 | 87.37 | 36.14 | 84.23 |
| 222 | | | 3.41 | 83.72 | 21.32 | 75.57 |
| 223 | | | 8.56 | 86.61 | 31.32 | 82.87 |
| 224 | | | 8.41 | 87.47 | 37.17 | 81.46 |
| 225 | | | 6.98 | 89.48 | 48.87 | 79.15 |
| 226 | | | 26.55 | 77.91 | 31.43 | 66.94 |
| 227 | | | 4.87 | 90.5 | 10.27 | 91.85 |
| 228 | | | 3.49 | 79.14 | 19.84 | 81.77 |
| 230 | | | 9.11 | 73.42 | 19.77 | 72.02 |
| 231 | | | 8.28 | 76.04 | 25.26 | 77.59 |
| 232 | | | 23.78 | 86.38 | 43.03 | 86.87 |
| 233 | | | 13.08 | 86.57 | 35.27 | 80.6 |
| 234 | | | 4.19 | 90.55 | 15.77 | 87.79 |
| 235 | | | 17.54 | 81.09 | 30.1 | 77.96 |
| 236 | | | 6.3 | 90.89 | 33.5 | 83.02 |
| 237 | | | 6.15 | 92.67 | 9.47 | 92.52 |
| 238 | | | 9.2 | 93.21 | 39.33 | 88.26 |
| 239 | | | 3.34 | 88.8 | 12.27 | 87.02 |
| 240 | | | 3.83 | 89.2 | 5.95 | 87.71 |
| 241 | | | 3.26 | 89.74 | 4.6 | 87.62 |
| 244 | | | 5.6 | 88.74 | 12.97 | 89.33 |
| 245 | | | 11.87 | 87.52 | 30.1 | 81.41 |
| 246 | | | 1.51 | 93.85 | 4.47 | 91.05 |
| 247 | | | 6.37 | 86.93 | 18.64 | 83.14 |
| 248 | | | 4.41 | 89.62 | 9.39 | 89.37 |
| 249 | | | 3.01 | 90.4 | 28.56 | 86.69 |
| 250 | | | 11.12 | 75.19 | 24.78 | 73.81 |
| 251 | | | 7.72 | 82.01 | 2.93 | 87.02 |
| 252 | | | 17.06 | 85.86 | 34.76 | 80.83 |
| 253 | | | 7.09 | 94.79 | 19.75 | 86.34 |
| 254 | | | 4.98 | 92.55 | 15.9 | 84.73 |
| 255 | | | 5.62 | 96.33 | 17.3 | 83.4 |
| 256 | | | 1.78 | 94.38 | 7.84 | 91.78 |
| 257 | | | 3.94 | 93.75 | 17.37 | 90.69 |
| 259 | >10000 | 11.68 | 3.72 | 95.97 | 7.04 | 92.12 |
| 264 | | | 17.15 | 74.58 | 61.02 | 86.24 |
| 266 | | | 50.27 | 83.88 | 81.89 | 73.56 |
| 267 | | | 17.92 | 93.96 | 47.71 | 90.27 |
| 268 | | | 22.02 | 93.75 | 33.46 | 90.47 |
| 269 | | | 21.53 | 95.39 | 43.71 | 88.84 |
| 270 | | | 6.97 | 91.36 | 24.04 | 84.18 |
| 272 | >10000 | 12.14 | 21.88 | 91.58 | 35.6 | 90.87 |
| 274 | | | 15.92 | 89.85 | 24.27 | 95.96 |
| 280 | | | 151.9 | 68.21 | 247.7 | 76.04 |
| 281 | | | 43.3 | 67.1 | 63.31 | 74.57 |
| 282 | | | 15.15 | 93.51 | 21.49 | 84.98 |
| 284 | | | 61.24 | 77.49 | 81.4 | 75.47 |
| 286 | | | 23.69 | 75.85 | 59.27 | 81.47 |
| 287 | | | 5.2 | 88.55 | 22.86 | 84.77 |
| 288 | | | 21.28 | 69.76 | 332.9 | 84.12 |
| 291 | | | 7.9 | 83.17 | 23.59 | 87.16 |
| 292 | | | 15.09 | 87.11 | 32.34 | 84.23 |
| 293 | | | 17.35 | 83.85 | 43.68 | 85.31 |
| 298 | | | 4.38 | 86.58 | 16.75 | 80.79 |
| 299 | | | 4.88 | 80.72 | 36.37 | 85.01 |
| 302 | | | 6.02 | 88.36 | 17 | 83.84 |
| 303 | | | 10.56 | 90.93 | 41.2 | 86.7 |
| 306 | | | 9.57 | 73.14 | 17.09 | 85.78 |
| 307 | | | 43.32 | 73.68 | 77.08 | 87.36 |
| 308 | | | 5.01 | 93.79 | 15.6 | 91.77 |
| 309 | | | 5.89 | 92.99 | 32.12 | 87.24 |
| 311 | | | 31.77 | 77.19 | 78.18 | 76.32 |
| 312 | | | 8.17 | 88.59 | 16.1 | 86.06 |
| 313 | | | 28.55 | 90.8 | 53.74 | 87.47 |
| 314 | | | 2.48 | 91.87 | 6.5 | 87.84 |
| 315 | | | 4.27 | 89.47 | 12.9 | 81.96 |
| 316 | | | 9.59 | 89.57 | 48.72 | 78.97 |
| 317 | | | 5.17 | 90.74 | 9.47 | 94.14 |
| 318 | | | 3.1 | 91.99 | 4.4 | 93.31 |
| 326 | | | 14.95 | 83.88 | 28.02 | 91.21 |
| 329 | | | 1.72 | 96.43 | 2.9 | 92.4 |
| 336 | | | 0.984 | 94.98 | 9.91 | 88.5 |
| 409 | | | 1.23 | 95.3 | 1.46 | 96.06 |
| 413 | | | 1.91 | 95.66 | 6.11 | 93.22 |
| 426 | | | 4.34 | 91.88 | 6.67 | 88.99 |
| 427 | | | 7.1 | 93.94 | 8.61 | 88.3 |
| 428 | | | 1.13 | 95.35 | 3.67 | 93.55 |
| 429 | | | 2.33 | 94.9 | 5.15 | 91.32 |
| 430 | | | 2.64 | 97.63 | 4.5 | 94.5 |
| 431 | | | 2.7 | 97.96 | 12.39 | 91.04 |
| 432 | | | 1.19 | 94.91 | 4.24 | 93 |
| 433 | | | 4.29 | 93.56 | 9.35 | 90.76 |
| 434 | | | 4.43 | 85.05 | 9.32 | 88.24 |
| 435 | | | 7.48 | 92.59 | 23.89 | 89.88 |
| 436 | | | 2.38 | 89.14 | 3.2 | 92.48 |
| 437 | | | 14.95 | 83.88 | 28.02 | 91.21 |
| 698 | | | 0.601 | 94.26 | 4.28 | 93.58 |
| 699 | | | 3.04 | 85.08 | 6.06 | 89.24 |
| 700 | | | 6.49 | 83.8 | 13.09 | 92.4 |
| 701 | | | 2.23 | 90.24 | 9.87 | 92.7 |
| 703 | | | 6.81 | 93.35 | 19.96 | 87.81 |
| 704 | | | 3.98 | 82.31 | 21.92 | 90.39 |
| 705 | | | 1.53 | 88.58 | 4.56 | 95.35 |
| 706 | | | 2.17 | 88.51 | 5.64 | 90.85 |
| 707 | | | 3.29 | 82.86 | 5.79 | 92.13 |
| 708 | | | 15.17 | 81.62 | 22.67 | 91.32 |
| 709 | | | 19.77 | 84.65 | 21.62 | 87.91 |
| 710 | | | 2.69 | 87.65 | 13.58 | 90.91 |
| 711 | | | 0.505 | 91.94 | 2.88 | 90.85 |
| 712 | | | 2.06 | 91.27 | 7.34 | 88.62 |
| 713 | | | 7.43 | 86.33 | 50.77 | 90.4 |
| 714 | | | 5.34 | 96.7 | 18.87 | 90.08 |
| 715 | | | 4.3 | 92.57 | 7.08 | 89.82 |
| 716 | | | 3.06 | 91.65 | 8.2 | 89.99 |
| 717 | | | 5.98 | 94.13 | 17.08 | 91.36 |
| 718 | | | 25.63 | 79.24 | 121.2 | 89.64 |
| 719 | | | 30.64 | 84.38 | 72.94 | 90.36 |
| 720 | | | 10.41 | 86.63 | 20.34 | 91.68 |
| 721 | | | 7.28 | 88.98 | 49.36 | 91.88 |
| 723 | | | 5.67 | 76.49 | 12.08 | 81.65 |

TABLE 2
Degradation (HCC827 and H1975 #36) result for Example 1 to
Example 318

| | H1975 #36 | | HCC827 | |
|---|---|---|---|---|
| Example | DC₅₀(nM) | Dmax (%) | DC₅₀(nM) | Dmax (%) |
| 1 | 7.22 | 60.84 | 3.6 | 86.93 |
| 2 | 3719 | 53.57 | 18.05 | 78.46 |
| 3 | >10000.0 | 45.13 | 14.34 | 72.27 |
| 4 | 7.44 | 75.37 | 11.07 | 83.62 |
| 5 | 4.66 | 84.13 | 2.56 | 83.9 |
| 7 | >156.0 | 49.01 | 3.95 | 74.27 |
| 10 | 15.89 | 82.64 | 12.35 | 83.3 |
| 11 | 5.41 | 97.42 | 2.84 | 86.4 |
| 13 | >10000.0 | 42.78 | 9.48 | 78.91 |
| 17 | >10000.0 | 6.77 | 25.42 | 73.32 |
| 18 | — | — | 15.39 | 77.49 |
| 22 | >10000.0 | 41.66 | 6.26 | 67.53 |
| 23 | 14.14 | 65.54 | 2.05 | 93.43 |
| 24 | 5.72 | 65.54 | 6.59 | 74.71 |
| 34 | 8.6 | 71.05 | 6.2 | 80.79 |
| 48 | 9.23 | 64.47 | 4.42 | 76.64 |
| 55 | >10000.0 | 18.68 | 312.8 | 56.18 |
| 56 | 6.5 | 88.75 | 2.63 | 93.43 |
| 57 | 5.31 | 77.37 | 4.8 | 76.07 |
| 58 | 3.9 | 88.48 | 5.45 | 78.53 |
| 59 | 1.62 | 82.52 | 4.68 | 85.22 |
| 60 | 16.54 | 73.43 | 4.8 | 86.8 |
| 61 | 9.34 | 77.16 | 2.84 | 89.99 |
| 62 | — | — | 6.24 | 83.72 |
| 63 | — | — | 1.74 | 86.34 |
| 64 | 3.12 | 85.77 | 2.06 | 75.73 |
| 65 | 2.31 | 85.6 | 2.53 | 73.9 |
| 67 | 9.74 | 74.83 | 6.2 | 76.71 |
| 68 | — | — | 13.12 | 64.26 |
| 74 | — | — | 57.19 | 85.23 |
| 75 | — | — | 50.6 | 72.14 |
| 76 | — | — | 30.51 | 77.06 |
| 77 | 2.77 | 63.03 | 9.18 | 70.87 |
| 78 | — | — | 15.25 | 70.06 |
| 79 | >10000 | 18.65 | 17.49 | 72.12 |
| 80 | — | — | 35.94 | 74.44 |
| 81 | >10000.0 | 5.13 | 25.8 | 68.77 |
| 82 | >10000.0 | 12.15 | 14.68 | 74.04 |
| 83 | >10000.0 | 14.65 | 20.27 | 74.18 |
| 84 | >10000.0 | 10.7 | 29.11 | 65.89 |
| 85 | >10000.0 | 20.85 | 73.33 | 75.04 |
| 86 | — | — | 66.73 | 80.99 |
| 87 | — | — | 57.51 | 86.18 |
| 88 | 3.54 | 54.52 | 21.91 | 68.66 |
| 89 | 6.23 | 84.74 | 59.62 | 91.76 |
| 90 | 6.53 | 77.46 | 12.8 | 81.42 |
| 91 | — | — | 90.43 | 73.49 |
| 92 | — | — | 306.3 | 76.83 |
| 93 | — | — | 129.6 | 72.38 |
| 94 | — | — | 32.17 | 58.51 |
| 95 | 9026.5 | 61.62 | 146.7 | 71.32 |
| 96 | >10000.0 | 7.3 | 151.3 | 67 |
| 97 | >10000.0 | 11.2 | 117.8 | 62.76 |
| 98 | >10000.0 | 8.3 | 166.2 | 62.28 |
| 99 | >10000.0 | 35.57 | 60.09 | 78.55 |
| 100 | 8.7 | 62.64 | 6.34 | 76.86 |
| 101 | — | — | 77.34 | 78.03 |
| 102 | 81.66 | 65.07 | 70.08 | 75.91 |
| 103 | 43.11 | 79.64 | 28.81 | 84.22 |
| 104 | 5.89 | 74.73 | 17.72 | 83.44 |
| 105 | 12.18 | 83.7 | 15.98 | 67.24 |
| 106 | 8.32 | 63.0 | 7.06 | 63.25 |
| 107 | — | — | 17.24 | 79.27 |
| 108 | — | — | 16.55 | 82.24 |
| 109 | — | — | 188.8 | 79.61 |
| 110 | — | — | 49.56 | 81.25 |
| 111 | — | — | 112.0 | 81.1 |
| 113 | 5.3 | 97.32 | 3.34 | 89.23 |
| 114 | — | — | 8.23 | 82.44 |
| 115 | 640.7 | 74.73 | 103.2 | 83.44 |
| 116 | 6.63 | 62.02 | 17.93 | 75.42 |
| 117 | 67.36 | 86.65 | 16.97 | 76.2 |
| 118 | 24.69 | 82.36 | 22.73 | 75.96 |
| 119 | 9.02 | 83.93 | 12.85 | 85.34 |
| 120 | 5.22 | 91.57 | 14.23 | 86.37 |
| 121 | — | — | 19.01 | 80.81 |
| 122 | — | — | 6.37 | 69.18 |
| 123 | 12.44 | 79.65 | 15.94 | 88.84 |
| 124 | — | — | 81.61 | 80.76 |
| 125 | >10000.0 | 27.24 | 103.1 | 73.45 |
| 126 | 2.61 | 83.93 | 19.08 | 84.03 |
| 127 | 11.94 | 64.74 | 35.94 | 71.37 |
| 128 | 11.53 | 85.01 | 3.2 | 84.02 |
| 129 | 9.29 | 85.41 | 11.07 | 87.25 |
| 130 | — | — | 45.81 | 80.81 |
| 131 | — | — | 68.3 | 80.14 |
| 132 | — | — | 34.05 | 79.99 |
| 133 | — | — | 39.08 | 78.18 |
| 134 | — | — | 38.03 | 77.6 |
| 136 | — | — | 68.46 | 79.51 |
| 137 | — | — | 9.41 | 92.55 |
| 138 | 3.58 | 87.02 | 15.08 | 82.15 |
| 139 | — | — | 53.61 | 75.69 |
| 140 | 5.24 | 73.27 | 8.56 | 88.88 |
| 141 | — | — | 19.21 | 82.05 |
| 142 | — | — | 6.22 | 89.4 |
| 143 | 5.43 | 56.24 | 12.07 | 72.97 |
| 160 | 3.07 | 76.78 | 2.69 | 81.67 |
| 168 | 1.61 | 62.4 | 4.85 | 81.25 |
| 203 | 7.24 | 95.8 | 9.06 | 89.89 |
| 219 | 0.65 | 69.6 | 3.21 | 65.91 |
| 220 | 8.8 | 85.58 | 5.1 | 77.74 |
| 221 | 6.46 | 61.03 | 5.15 | 75.31 |
| 222 | 7.03 | 68.95 | 3.71 | 78.5 |
| 247 | 5.1 | 72.83 | 4.59 | 67.02 |
| 254 | 3.61 | 75.45 | 3.14 | 77.65 |
| 255 | 3.27 | 90.17 | 2.87 | 78.55 |
| 259 | 4.69 | 99.92 | 5.33 | 80.95 |
| 270 | 17.08 | 84.64 | 5.43 | 88.97 |
| 272 | 10.54 | 74.97 | 5.35 | 86.21 |
| 317 | >80.0 | 46.99 | 0.779 | 51.62 |
| 318 | 2.14 | 88.82 | 0.601 | 85.16 |

TABLE 3
Degradation (H1975 # 28 DTC and BaF3-LTC) result for Example 441
to Example 756

| | H1975 # 28 DTC | | BaF3-LTC | |
|---|---|---|---|---|
| Example | DC₅₀(nM) | Dmax (%) | DC₅₀(nM) | Dmax (%) |
| 441 | 0.887 | 79.34 | 7.93 | 92.5 |
| 442 | 1.05 | 81.11 | 6.53 | 86.95 |
| 443 | 0.894 | 82.38 | 14.57 | 81.38 |
| 444 | 1.19 | 79.58 | 6.28 | 79.39 |
| 445 | 0.877 | 79.2 | 5.24 | 87.69 |
| 446 | 0.726 | 80.48 | 3.89 | 96.37 |
| 447 | 0.526 | 77.96 | 2.27 | 83.22 |
| 448 | 0.962 | 83.19 | 6.92 | 89.53 |
| 449 | 0.937 | 85.59 | 2.99 | 89.93 |
| 450 | 0.616 | 77 | 2.68 | 87.69 |
| 451 | 0.612 | 70.68 | 3.64 | 95.36 |
| 452 | 0.863 | 78.26 | 3.96 | 89.74 |
| 453 | 0.704 | 77.04 | 5.7 | 93.14 |
| 454 | 0.735 | 78.41 | 5.13 | 92.34 |
| 455 | 0.47 | 79.19 | 3.83 | 87.13 |
| 456 | 0.621 | 71.36 | 3.56 | 89.92 |
| 457 | 0.655 | 74.18 | 3.25 | 92.54 |
| 458 | 0.505 | 83.62 | 2.09 | 88.26 |
| 459 | 0.851 | 86.08 | 7.54 | 86.94 |
| 460 | 2.43 | 86 | 16.4 | 82.54 |
| 461 | 38.77 | 75.83 | 132.2 | 90.04 |
| 462 | 1.12 | 78.37 | 19.19 | 84.53 |
| 463 | 0.26 | 82.73 | 3.34 | 92.59 |
| 464 | 0.693 | 73.89 | 9.8 | 78.23 |
| 465 | 1.92 | 70.68 | 2.46 | 84.48 |
| 466 | 2.03 | 72.67 | 15.09 | 90.84 |
| 467 | 1.83 | 76.06 | 12.49 | 91.3 |
| 468 | 0.998 | 74.46 | 12.24 | 90.18 |
| 469 | 2.6 | 81.32 | 11.6 | 89.97 |
| 470 | >10000 | 39.87 | 417.7 | 80.47 |
| 471 | 1.98 | 73.66 | 35.95 | 83.91 |
| 472 | 0.945 | 80.01 | 19.6 | 86.87 |
| 473 | 0.616 | 83.08 | 9.51 | 94.64 |
| 474 | 1.15 | 73.43 | 11.98 | 79.25 |
| 476 | 7.95 | 74.62 | 26.47 | 88.04 |
| 477 | 4.24 | 73.35 | 7.16 | 82.65 |
| 478 | 0.645 | 80.49 | 3.05 | 88.22 |
| 479 | 2.81 | 95.6 | 7.8 | 88.77 |
| 480 | 7.82 | 69.54 | 18.92 | 89.38 |
| 481 | 2.14 | 77.65 | 5.27 | 86.47 |
| 482 | 1.86 | 69.65 | 12.86 | 88.73 |
| 483 | 2.02 | 81.31 | 26.62 | 86.92 |
| 484 | 0.781 | 89.6 | 6.77 | 95.43 |
| 485 | 2.78 | 70.65 | 26.89 | 88.53 |
| 486 | 0.802 | 85.73 | 5.83 | 87.82 |
| 487 | 2.22 | 85.45 | 5.25 | 98.02 |
| 488 | 0.97 | 84.91 | 3.52 | 96.59 |
| 489 | 1.37 | 82.72 | 8.99 | 98.64 |
| 490 | 0.2 | 80.5 | 1.3 | 92.8 |
| 491 | 0.647 | 78.48 | 2.72 | 92.69 |
| 492 | 0.605 | 70.88 | 3.39 | 95.55 |
| 493 | 0.588 | 70.43 | 2.71 | 95.18 |
| 494 | 0.637 | 79.15 | 1.73 | 92.07 |
| 495 | 1.15 | 83.29 | 7.31 | 89.24 |
| 496 | 0.92 | 75.32 | 2.3 | 85.49 |
| 497 | 1.01 | 80.94 | 6.27 | 86.62 |
| 498 | 2.1 | 86.84 | 7.62 | 91.41 |
| 499 | 0.185 | 77.41 | 1.95 | 87.82 |
| 500 | 0.413 | 71.9 | 2.42 | 84.81 |
| 501 | 0.759 | 85.93 | 3.34 | 83.49 |
| 502 | 0.404 | 88.96 | 3.02 | 96.68 |
| 503 | 1.34 | 79.81 | 7 | 90.24 |
| 504 | 0.962 | 75.99 | 6.56 | 89.03 |
| 505 | 0.925 | 77.6 | 9.43 | 75.06 |
| 506 | 0.96 | 84.41 | 6.1 | 92.96 |
| 507 | 0.5 | 68.8 | 4.3 | 89.8 |
| 508 | 1.0 | 72.7 | 14.7 | 89.3 |
| 509 | 0.887 | 79.34 | 7.93 | 92.5 |
| 510 | 0.763 | 67.2 | 3.89 | 93.08 |
| 511 | 1.04 | 65 | 10.62 | 92.53 |
| 512 | 2.36 | 60.54 | 6.46 | 91.95 |
| 513 | 1.37 | 78.24 | 10.21 | 85.15 |
| 514 | 0.771 | 66.48 | 3.51 | 94.34 |
| 515 | 3.22 | 66.35 | 19.71 | 90.36 |
| 516 | 0.612 | 70.68 | 3.64 | 95.36 |
| 517 | 0.968 | 58.8 | 7 | 91.31 |
| 518 | 0.986 | 68.67 | 16.06 | 92.17 |
| 519 | 0.378 | 74.38 | 1.19 | 92.07 |
| 520 | 0.769 | 73.99 | 5.66 | 90.12 |
| 521 | 1.01 | 80.24 | 16.96 | 87.44 |
| 522 | 0.611 | 75.25 | 1.43 | 95.31 |
| 523 | 0.571 | 72.16 | 1.97 | 95.94 |
| 524 | 0.717 | 76.34 | 3.64 | 96.98 |
| 525 | 0.691 | 50.86 | 2.6 | 94.29 |
| 526 | 0.574 | 83.99 | 3.77 | 96.35 |
| 527 | 0.583 | 85.27 | 1.77 | 93.4 |
| 528 | 0.632 | 86.11 | 3.63 | 97.27 |
| 529 | 1.03 | 85.06 | 5.8 | 91.93 |
| 530 | 1.87 | 82.54 | 5.05 | 94.61 |
| 531 | 1.55 | 74.06 | 7.03 | 96.42 |
| 532 | 0.642 | 70.91 | 2.96 | 95.58 |
| 533 | 1.18 | 83.78 | 5.05 | 97.23 |
| 534 | 0.749 | 83.48 | 4.2 | 93.68 |
| 535 | 1.13 | 82.79 | 9.87 | 93.85 |
| 536 | 0.582 | 73.22 | 0.867 | 96.27 |
| 537 | 0.223 | 65.01 | 0.934 | 94.02 |
| 538 | 2.39 | 80.57 | 16.16 | 89.57 |
| 539 | 1.09 | 84.42 | 6.44 | 94.1 |
| 541 | 2.08 | 88.77 | 17.78 | 95.63 |
| 542 | 0.75 | 45.37 | 11.24 | 95.6 |
| 543 | 0.851 | 86.08 | 7.54 | 86.94 |
| 544 | 1.48 | 81.13 | 12.5 | 90.31 |
| 545 | 0.666 | 75.11 | 8.37 | 91 |
| 546 | 0.65 | 69.09 | 12.07 | 97.68 |
| 547 | 0.647 | 87.96 | 4.45 | 89.32 |
| 548 | 0.696 | 74.38 | 3.63 | 91.75 |
| 549 | 0.601 | 88.85 | 3.1 | 90.37 |
| 550 | 0.707 | 87.71 | 8.12 | 85.9 |
| 551 | 1.62 | 90.57 | 6.19 | 89.72 |
| 552 | 0.367 | 71.85 | 4.35 | 85.68 |
| 553 | 0.712 | 61.54 | 2.49 | 90.71 |
| 554 | 0.662 | 67.4 | 2.49 | 91.94 |
| 555 | 1.89 | 77.45 | 7.36 | 94.47 |
| 556 | 4.03 | 98.64 | 14.83 | 92.23 |
| 557 | 0.773 | 92.3 | 6.8 | 89.14 |
| 558 | 2.59 | 85.18 | 13.09 | 95.88 |
| 559 | 0.364 | 80.93 | 12.03 | 92.42 |
| 560 | 1.36 | 80.68 | 14.09 | 92.29 |
| 561 | 1.68 | 77.5 | 10.34 | 89.47 |
| 562 | 1.54 | 77.73 | 7.47 | 90.16 |
| 563 | 0.732 | 81.88 | 7.69 | 92.58 |
| 564 | 1.04 | 81.87 | 7.34 | 93.91 |
| 565 | 0.626 | 74.32 | 3.92 | 93.15 |
| 566 | 0.616 | 84.02 | 5.25 | 93.94 |
| 567 | 3.01 | 56.23 | 13.18 | 91.96 |
| 568 | 2.38 | 91.62 | 10.37 | 87.01 |
| 569 | 0.595 | 62.25 | 7.8 | 86.46 |
| 570 | 3.03 | 76.54 | 20.47 | 91.68 |
| 571 | 0.946 | 77.08 | 3.49 | 93.17 |
| 572 | 1.81 | 83.86 | 3.21 | 90.33 |
| 573 | 0.751 | 81.91 | 3.36 | 97.7 |
| 574 | 1.62 | 90.57 | 6.19 | 89.72 |
| 575 | 2.08 | 88.77 | 17.78 | 95.63 |
| 576 | 2.8 | 81.93 | 12.14 | 92.21 |
| 577 | 1.08 | 87.91 | 8.4 | 88.6 |
| 578 | 3.11 | 87.5 | 12.22 | 96.55 |
| 581 | 1.36 | 72.5 | 6.9 | 88.48 |
| 582 | 1.06 | 79.64 | 8.54 | 93.07 |
| 583 | 2.7 | 76.91 | 17.01 | 92.92 |
| 584 | 0.634 | 79.5 | 2.72 | 95.57 |
| 585 | 0.695 | 79.08 | 2.5 | 96.24 |
| 586 | 0.958 | 84.73 | 3.85 | 94.09 |
| 587 | 0.765 | 87.02 | 2.19 | 89.2 |
| 588 | 1.94 | 84.46 | 18.42 | 87.49 |
| 589 | 0.844 | 86.23 | 4.34 | 85.94 |
| 590 | 0.697 | 87.82 | 11.82 | 91.71 |
| 591 | 1.57 | 75.79 | 72.75 | 74.97 |
| 592 | 1.18 | 82.2 | 12.5 | 83.74 |
| 593 | 1.26 | 76.9 | 13.21 | 82.64 |
| 594 | 0.818 | 85.75 | 16.99 | 85.45 |
| 595 | 1.02 | 72.7 | 16.13 | 81.98 |
| 596 | 0.937 | 84.17 | 6.56 | 91.42 |
| 597 | 1.59 | 75.93 | 28.24 | 90.09 |
| 598 | 1.64 | 81.26 | 18.78 | 88.42 |
| 599 | 1.91 | 80.53 | 25.01 | 73.91 |
| 600 | 1.18 | 83.32 | 4.11 | 87.43 |
| 601 | 0.632 | 77.35 | 8.64 | 89.49 |
| 602 | 0.913 | 77.98 | 3.66 | 92.97 |
| 603 | 1.57 | 81.2 | 7.62 | 91.19 |
| 604 | 0.764 | 69.1 | 18.5 | 86.14 |
| 607 | 1.07 | 78.76 | 14.25 | 86.84 |
| 609 | 3.07 | 74.7 | 11.48 | 89.57 |
| 611 | 5.69 | 70.37 | 83.37 | 86.27 |
| 612 | 0.656 | 72.14 | 7.68 | 93.22 |
| 613 | 3.55 | 71.47 | 17.34 | 88.61 |
| 614 | 2.61 | 74 | 23.82 | 80.74 |
| 615 | 0.921 | 72.36 | 7.91 | 90.99 |
| 616 | 5.75 | 70.93 | 43.14 | 83.84 |
| 617 | 1.15 | 74.31 | 5.77 | 91.31 |
| 618 | 2.14 | 74.88 | 53.18 | 74.15 |
| 619 | 0.91 | 62.88 | 11.17 | 92.11 |
| 620 | 2.04 | 78.78 | 8.28 | 93.76 |
| 621 | 1.61 | 77.44 | 14.98 | 93.93 |
| 622 | 4.78 | 75.46 | 37.31 | 94.03 |
| 623 | 1.31 | 75.76 | 17.11 | 92.03 |
| 624 | 1.05 | 73.81 | 21.92 | 95.44 |
| 625 | 1.86 | 80.1 | 10.96 | 88.79 |
| 626 | 0.711 | 76.73 | 5.43 | 91.04 |
| 627 | 1.23 | 76.26 | 9.13 | 94.47 |
| 628 | 0.715 | 76.12 | 3.24 | 92.34 |
| 629 | 0.965 | 71.58 | 7.66 | 89.55 |
| 630 | 1.14 | 72.34 | 8.65 | 91.04 |
| 631 | 1.04 | 82.5 | 9.33 | 86.8 |
| 632 | 2.2 | 81.77 | 14.22 | 95.09 |
| 633 | 0.737 | 70.09 | 5.12 | 91.73 |
| 634 | 0.985 | 81.71 | 4.47 | 87.97 |
| 635 | 1.37 | 77.57 | 11.03 | 90.66 |
| 636 | 1.52 | 79.04 | 10.95 | 92.34 |
| 637 | 0.354 | 69.46 | 5.39 | 92.88 |
| 639 | 0.71 | 86.93 | 4.06 | 94.32 |
| 640 | 3.09 | 85.38 | 17.9 | 87.21 |
| 641 | 0.666 | 76.33 | 7.79 | 96.06 |
| 642 | 0.617 | 76.78 | 1.42 | 93.67 |
| 643 | 2.48 | 65.15 | 3.86 | 91.19 |
| 644 | 0.829 | 86.29 | 5.12 | 92.61 |
| 645 | 6.2 | 84.26 | 22.74 | 95.76 |
| 646 | 0.627 | 74.64 | 6.2 | 94.5 |
| 647 | 2 | 66.66 | 13.17 | 93.2 |
| 648 | 4.01 | 81.9 | 15.28 | 95.82 |
| 649 | 6.94 | 75.95 | 31.83 | 78.62 |
| 650 | 3.39 | 59.93 | 16.05 | 75.02 |
| 651 | 1.17 | 79.33 | 2.13 | 87.74 |
| 652 | 1.35 | 86.65 | 5.23 | 81.06 |
| 653 | 0.7 | 87.89 | 7.19 | 87.71 |
| 654 | 2.08 | 66.53 | 23.24 | 74.81 |
| 655 | 4.21 | 81.86 | 16.39 | 90.16 |
| 656 | 1.66 | 83.91 | 5.22 | 83.58 |
| 657 | 0.981 | 86.35 | 3.3 | 87.67 |
| 658 | 0.59 | 71.03 7 | 4.46 | 84.15 |
| 659 | 2.8 | 77.5 | 17.8 | 73.34 |
| 660 | 2.27 | 77.55 | 15.89 | 72.22 |
| 661 | 0.609 | 87.94 | 8.68 | 85.78 |
| 662 | 2.23 | 89.14 | 5.4 | 96.96 |
| 663 | 1.81 | 74.11 | 3.98 | 95.8 |
| 665 | 0.543 | 85.47 | 1.49 | 97.86 |
| 666 | 2.4 | 86.11 | 11.15 | 93.23 |
| 667 | 3.69 | 77.43 | 13.26 | 84.01 |
| 668 | 0.579 | 86.5 | 2.32 | 91.55 |
| 669 | 0.59 | 73.26 | 4.06 | 91.14 |
| 670 | 0.634 | 83.95 | 3.37 | 88.3 |
| 671 | 0.219 | 75.9 | 4.85 | 92.72 |
| 672 | 0.723 | 87.05 | 6.6 | 86.9 |
| 673 | 0.677 | 85.2 | 5.79 | 93.27 |
| 674 | 2.19 | 69.95 | 9.62 | 94.61 |
| 675 | 1.01 | 82.47 | 4.3 | 96.47 |
| 676 | 0.64 | 88.89 | 3.14 | 96.39 |
| 677 | 3.83 | 77.05 | 38.04 | 94.34 |
| 678 | 1.39 | 81.53 | 5.8 | 94.94 |
| 679 | 1.75 | 83.46 | 22.55 | 93.28 |
| 680 | 0.763 | 85.2 | 4.96 | 95.13 |
| 681 | 1.37 | 77.05 | 4.65 | 96.04 |
| 682 | 0.91 | 87.08 | 6.79 | 94.97 |
| 683 | 0.595 | 75.35 | 1.11 | 95.9 |
| 684 | 1.06 | 88.35 | 12.38 | 91.43 |
| 685 | 0.787 | 83.23 | 5.29 | 95.01 |
| 686 | 1.76 | 88.73 | 4.89 | 96.64 |
| 687 | 1.38 | 68.75 | 14.82 | 93.28 |
| 689 | 3.03 | 72.37 | 15.37 | 85.11 |
| 690 | 0.689 | 77.81 | 6.66 | 90.58 |
| 691 | 3.3 | 71.66 | 24.58 | 93.4 |
| 692 | 0.593 | 73 | 6.68 | 91.36 |
| 693 | 0.812 | 66.27 | 13.77 | 84.52 |
| 694 | 0.986 | 68.67 | 16.06 | 92.17 |
| 695 | 3.06 | 68.81 | 12.73 | 93.46 |
| 696 | 0.647 | 75.49 | 4.53 | 92.34 |
| 697 | 1.19 | 71.39 | 9.65 | 94.11 |
| 724 | 2.86 | 75.94 | 55.08 | 85.56 |
| 726 | 1.46 | 74.13 | 7.11 | 96.72 |
| 727 | 4.29 | 77.39 | 8.61 | 97.51 |
| 728 | 0.803 | 87.57 | 9.14 | 95.35 |
| 729 | 1.13 | 88.94 | 5.59 | 97.07 |
| 730 | 8.9 | 81.41 | 81.14 | 83.73 |
| 731 | 4.55 | 93.44 | 42.51 | 80.31 |
| 732 | 0.624 | 86.49 | 4.07 | 95.45 |
| 733 | 7.25 | 84.52 | 89.87 | 89.45 |
| 734 | 0.9 | 76.3 | 7.79 | 92.15 |
| 735 | 0.641 | 80.09 | 18.04 | 87.38 |
| 736 | 0.564 | 81.93 | 4.01 | 97.03 |
| 737 | 0.944 | 78.21 | 3.72 | 97.71 |
| 738 | 0.64 | 84.3 | 6.73 | 95.67 |
| 739 | 0.614 | 85.55 | 2.89 | 95.69 |
| 740 | 0.717 | 87.27 | 4.11 | 97.19 |
| 741 | 0.761 | 80.67 | 11.19 | 96.56 |
| 742 | 3.52 | 68.91 | 36.52 | 78.08 |
| 743 | 2.42 | 75.96 | 55.91 | 79.73 |
| 744 | 0.653 | 84.24 | 5.87 | 92.31 |
| 745 | 2.25 | 75.21 | 40.27 | 88.73 |
| 746 | 11.04 | 77.61 | 30.85 | 95.54 |
| 747 | 2.91 | 76.51 | 19.75 | 96.2 |
| 748 | 0.455 | 91.78 | 5.75 | 93.6 |
| 749 | 14.77 | 66.03 | 293.7 | 35.87 |
| 750 | 1.17 | 84.8 | 5.23 | 95.42 |
| 751 | 0.759 | 92.19 | 9.43 | 94.61 |
| 752 | 1.0 | 77.12 | 6.34 | 94.69 |
| 753 | 6.28 | 87.49 | 83.65 | 93.22 |
| 754 | 3.22 | 86.62 | 13.41 | 96.29 |
| 755 | 0.57 | 84.62 | 4.13 | 98.58 |
| 756 | 0.608 | 80.03 | 2.31 | 97.05 |

TABLE 4
Degradation (H1975 # 23 DC and BaF3-LC) result for Example 441
to Example 756

| | H1975 #23 DC | | BaF3 LC | |
|---|---|---|---|---|
| Example | DC₅₀(nM) | Dmax (%) | DC₅₀(nM) | Dmax (%) |
| 441 | 1.94 | 77.77 | 15.49 | 80.36 |
| 446 | 0.616 | 59.25 | 5.87 | 81.68 |
| 451 | 1.11 | 78.66 | 1.83 | 94.78 |
| 455 | 2.31 | 77.73 | 121.3 | 77.76 |
| 458 | 3.26 | 59.65 | 3.29 | 91.55 |
| 459 | 6.01 | 91.45 | 20.66 | 76.61 |
| 460 | 3.11 | 82.75 | 37.27 | 88.23 |
| 462 | 3.19 | 77.67 | 67.71 | 89.89 |
| 463 | 0.951 | 56.75 | 6.88 | 85.53 |
| 465 | >16.0 | 48.97 | 1.94 | 92.69 |
| 468 | 11.95 | 79.65 | 66.13 | 82.85 |
| 469 | 1.48 | 73.64 | 21.66 | 89.77 |
| 470 | 9538.1 | 64.89 | 300.4 | 86.64 |
| 473 | 12.76 | 82.45 | 73.39 | 85.48 |
| 474 | 2.65 | 68.97 | 30.36 | 81.06 |
| 479 | 4.91 | 76.53 | 20.31 | 82.67 |
| 481 | 1.06 | 64.14 | 0.871 | 91.9 |
| 482 | 7.2 | 70.91 | 50.83 | 73.44 |
| 483 | 7.19 | 70.71 | 68.97 | 83.21 |
| 484 | 3.54 | 82.99 | 11.92 | 91.56 |
| 485 | 10.4 | 82.7 | 35.1 | 88.3 |
| 486 | 4.73 | 80.02 | 16.59 | 87.83 |
| 487 | 4.02 | 74.41 | 10.12 | 95.61 |
| 488 | 2.23 | 67.73 | 17.86 | 92.82 |
| 489 | 4.72 | 59.94 | 19.73 | 86.62 |
| 490 | 0.974 | 64.67 | 2.41 | 95.65 |
| 491 | 0.85 | 58.85 | 2.34 | 94.72 |
| 492 | 3.07 | 51.44 | 4.28 | 79.45 |
| 493 | 11.15 | 63.11 | 24.32 | 87.08 |
| 494 | 0.706 | 64.02 | 5.41 | 92.91 |
| 495 | 1.78 | 79.88 | 9.64 | 91.42 |
| 496 | 0.787 | 95.59 | 2.23 | 91.34 |
| 497 | 3.32 | 74.87 | 5.19 | 93.35 |
| 498 | 0.377 | 66.76 | 0.718 | 92.92 |
| 499 | 0.646 | 61.96 | 0.955 | 90.95 |
| 500 | 1.11 | 55.74 | 1.11 | 91.95 |
| 501 | 3.29 | 65.52 | 1.49 | 93.48 |
| 502 | 5.49 | 75.93 | 57.17 | 87.59 |
| 503 | 1.57 | 81.03 | 7.53 | 88.86 |
| 504 | 2.95 | 78.25 | 12.45 | 86.05 |
| 505 | 3.17 | 68.68 | 8.88 | 83.25 |
| 506 | 2.04 | 80.87 | 22.47 | 91.68 |
| 507 | 2.41 | 52.12 | 8.97 | 94.02 |
| 509 | 1.94 | 77.77 | 15.49 | 80.36 |
| 511 | 3.3 | 67.01 | 9.67 | 88.86 |
| 514 | 1.83 | 76.66 | 3.51 | 94 |
| 516 | 1.11 | 78.66 | 1.83 | 94.78 |
| 521 | 5.9 | 75.56 | 51.62 | 74.59 |
| 524 | 1.23 | 67.29 | 5.62 | 95.66 |
| 526 | 5.87 | 52.26 | 40.33 | 74.56 |
| 528 | 1.58 | 54.87 | 14.91 | 83.93 |
| 531 | 9.42 | 53.36 | 51.82 | 83.5 |
| 532 | 0.591 | 52.49 | 3.84 | 88.09 |
| 533 | 7.47 | 73.47 | 28.77 | 90.76 |
| 534 | 2.16 | 80.71 | 10.23 | 90.25 |
| 543 | 6.01 | 91.45 | 20.66 | 76.61 |
| 545 | 2.07 | 72.65 | 4.6 | 89.57 |
| 549 | 2.96 | 63.66 | 1.59 | 87.15 |
| 551 | 5.64 | 84.14 | 46.47 | 86.8 |
| 553 | 0.828 | 53.56 | 1.23 | 93.29 |
| 559 | 5.38 | 73.67 | 26.91 | 90.43 |
| 571 | 3.05 | 75.83 | 13.19 | 87.65 |
| 572 | 4.74 | 69.16 | 3.8 | 91.75 |
| 574 | 5.64 | 84.14 | 46.47 | 86.8 |
| 576 | 3.59 | 77.19 | 15.82 | 95.93 |
| 577 | 7.94 | 64.13 | 53.78 | 89.1 |
| 584 | 2.7 | 70.66 | 4.9 | 92.75 |
| 587 | 6.14 | 87.43 | 14.57 | 87.8 |
| 589 | 2.2 | 84.51 | 20.27 | 92.36 |
| 643 | 1.45 | 73.67 | 3.97 | 94.1 |
| 750 | 6.52 | 74.02 | 26.13 | 94.35 |
| 752 | 1.76 | 78.99 | 2.23 | 93.97 |
| 753 | 2.43 | 78.45 | 20.07 | 88.89 |
| 754 | 5.6 | 71.73 | 36.07 | 87.35 |
| 755 | 7.68 | 69.09 | 16.38 | 91.74 |
| 756 | 1.69 | 73.8 | 10.06 | 94.5 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

What is claimed is:

1. A compound of Formula (I): 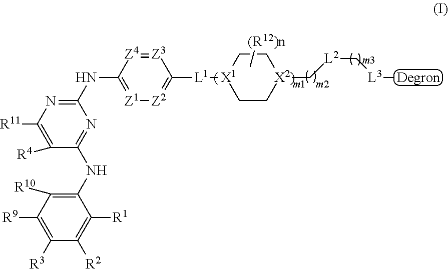
or a N-oxide thereof, or a pharmaceutically acceptable salt thereof, or a stereoisomer thereof, or a deuterated analog thereof, or a prodrug thereof
wherein:
R¹ is selected from —P(O)R^{1a}R^{1b}, —SO₂R^{1a}, —SO₂—NR^{1a}R^{1b} or —N(R^{1a})—SO₂R^{1b};
R^{1a} and R^{1b} are each independently selected from hydrogen, halogen, —C₁-C₈alkyl or C₃-C₈cycloalkyl, said —C₁-C₈alkyl or C₃-C₈cycloalkyl is optionally substituted with at least one halogen;
R² and R³ are each independently selected from hydrogen, halogen, —C₁-C₈alkyl, —C₃-C₈cycloalkyl, 3- to 8-membered heterocyclyl, C₆-C₁₂aryl, 5- to 12-membered heteroaryl, —CN, —OR^{2a}, —SO₂R^{2a}, —SO₂NR^{2a}R^{2b}, —C(O)R^{2a}, —CO₂R^{2a}, —C(O)NR^{2a}R^{2b}, —NR^{2a}R^{2b}, —NR^{2a}C(O)R^{2b}, —NR^{2a}CO₂R^{2b}, or —NR^{2a}SO₂R^{2b}; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{2d}, or
R² and R³ together with the carbon atoms to which they are attached, form a 5 or 6-membered unsaturated or saturated ring, said ring comprising 0-3 heteroatoms independently selected from nitrogen, oxygen or sulfur; said ring is optionally substituted with at least one substituent R^{2e};
R^{2e}, at each occurrence, is independently hydrogen, halogen, —C₁-C₈alkyl, —C₁-C₈alkoxy, —C₃-C₈cycloalkyl, 3- to 8-membered heterocyclyl, C₆-C₁₂aryl, 5- to 12-membered heteroaryl, oxo (═O), —OR^{2a}, thioxo (═S), —SR^{2a}, —CN, —SO₂R^{2a}, —SO₂NR^{2a}R^{2b}, —C(O)R^{2a}, —CO₂R^{2a}, —C(O)NR^{2a}R^{2b}, —NR^{2a}R^{2b}, —NR^{2a}COR^{2b}, —NR^{2a}CO₂R^{2b} or —NR^{2a}SO₂R^{2b}; each of —C₁-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent R^{2d};
R^{2a} and R^{2b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;
R^{2d}, at each occurrence, is independently halogen, —OH, —CN, oxo (═O), —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R⁴ is selected from hydrogen, halogen, —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl, 5- to 12-membered heteroaryl, —CN, —SO₂R^{4a}, —SO₂NR^{4a}R^{4b}, —C(O)R^{4a}, —CO₂R^{4a}, —C(O)NR^{4a}R^{4b}, —NR^{4a}R^{4b}, —NR^{4a}COR^{4b}, —NR^{4a}CO₂R^{4b} or —NR^{4a}SO₂R^{4b}; each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo (═O), —CN, —OR^{4c}, —SO₂R^{4c}, —SO₂NR^{4c}R⁴—, —C(O)R^{4c}, —CO₂R^{4c}, —C(O)NR^{4c}R^{4d}, —NR^{4c}R^{4d}, —NR^{4c}COR^{4d}, —NR^{4c}CO₂R^{4d} or —NR^{4c}SO₂R^{4d};
R^{4a}, R^{4b}, R^{4c} and R^{4d} are each independently hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁹, R¹⁰, R¹¹ and R¹² are each independently selected from hydrogen, halogen, —C₁-C₈alkyl, —NR^{9a}R^{9b}, —OR^{9a}, —C₃-C₈cycloalkyl, 3- to 8-membered heterocyclyl, —C₆-C₁₂aryl, 5- to 12-membered heteroaryl, oxo (═O) or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{9c}; or
two R¹² together with the carbon atoms to which they are attached, form a 3- to 12-membered ring, said ring comprising 0-3 heteroatoms independently selected from nitrogen, oxygen or sulfur; said ring is optionally substituted with at least one substituent R^{9c};
R^{9a} and R^{9b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5 to 12-membered heteroaryl is optionally substituted with at least one substituent R^{9d}; or
R^{9c} and R^{9d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl;
Z¹, Z², Z³ and Z⁴ are each independently selected from —CR^{Z}, or N;
R^{Z}, at each occurrence, is independently selected from hydrogen, halogen, —C₁-C₈alkyl, —NR^{Za}R^{Zb}, —OR^{Za}, —SR^{Za}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{Zd};
R^{Zc} and R^{Zd} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5 to 12-membered heteroaryl is optionally substituted with at least one substituent R^{Zd};
R^{Zc} and R^{Zd} are each independently selected from halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
L¹ is selected from a single bond, —O—, —SO₂—, —C(O)—, —NR^{L1a}—, —C₃-C₈cycloalkylene-, *^{L1}—O—C C₈alkylene-**^{L1}, *^{L1}—C₁-C₈alkylene-O—**^{L1}, *^{L1}—SO₂—C₁-C₈alkylene-**^{L}” *^{L1}—C₁-C₈alkylene-SO₂—**^{L1}, *^{L1}—C(O)—C₁-C₈alkylene-**^{L1}, *^{L1}—C₁-C₈alkylene-C(O)—**^{L1}, *^{L1}—NR^{L1a}—C₁-C₈alkylene-**^{L1}, *^{L1}—C₁-C₈alkylene- NR^{L1a}-**^{L1}, *^{L1}—NR^{L1a} C(O)—**^{L1}, *^{L1}—C(O)NR^{L1a}—**^{L1}, —C₁-C₈alkylene-, —C₂-C₈alkenylene-, —C₂-C₈alkynylene-, —[O(CR^{L1a}R^{L1b})ₘ₄]ₘ₅ 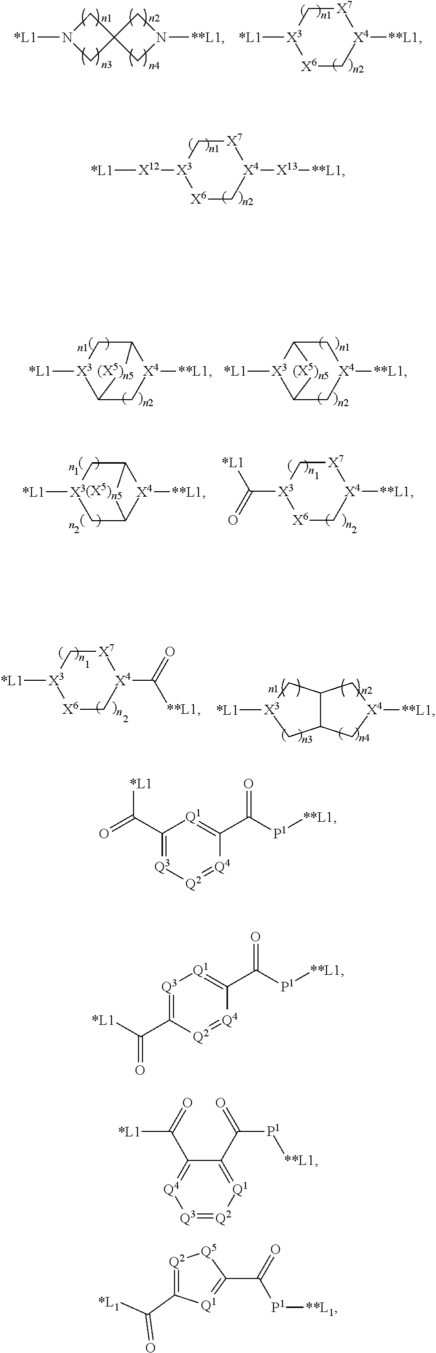 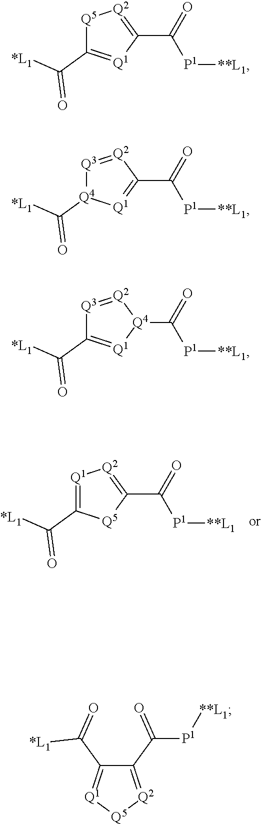
wherein each of said —C₃-C₈cycloalkylene-, *^{L1}—OC₁-C₈alkylene-**^{L1},*^{L1}—C₁-C₈alkylene-O—**^{L1}, *^{L1}—SO₂—C₁-C₈alkylene-**^{L1}, *^{L1}—C₁-C₈alkylene-SO₂— **^{L1}, *^{L1}—C(O)—C₁-C₈alkylene-**^{L1}, *^{L1}—C₁-C₈alkylene-C(O)—**^{L1}, *^{L1}—NR^{Lla}—C₁-C₈alkylene-**^{L1}, *^{L1}—C₁-C₈alkylene-NR^{L1a}—**^{L1}—C₁-C₈alkylene-, —C₂-C₈alkenylene-, —C₂-C₈alkynylene-, 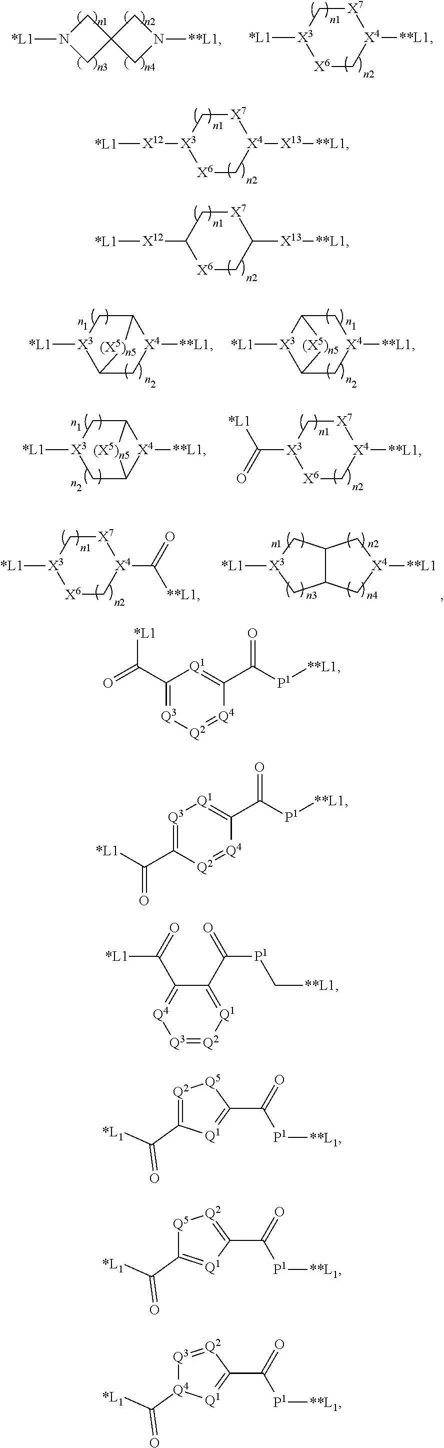 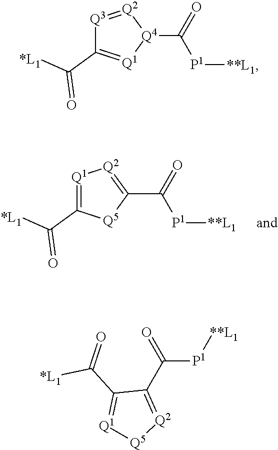
is optionally substituted with at least one R^{L1c};
wherein *^{L1} refers to the position attached to the 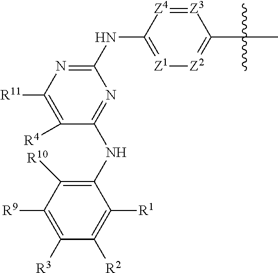
moiety, and **^{L1} refers to the position attached to the 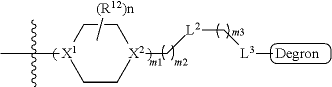
moiety;
R^{L1a} and R^{L1b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L1d};
each of said R^{L1c} and R^{L1d} are independently oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
L² is selected from a single bond, —O—, —SO₂—, —C(O)—, —NR^{L2a}—, —C₃-C₈cycloalkylene-, *^{L1}—O—C₁-C₈alkylene-**^{L2}, *^{L2}—C₁-C₈alkylene-O—**^{L2}, *^{L2}—SO₂—C₁-C₈alkylene-**^{L2}, *^{L2}—C₁-C₈alkylene-SO₂—**^{L2}, *L C(O)—C₁-C₈alkylene-**^{L2}, *^{L2}—C₁-C₈alkylene-C(O)—**^{L2}, *^{L2}NR^{L2a}—C₁-C₈alkylene-**^{L2}, *^{L2}—C₁-C₈alkylene-NR^{L2a}—**^{L2}, *^{L2}NR^{L2a}C(O)—**^{L2}, *^{L2}—C(O)NR^{L2a}—**^{L2}, C₁-C₈alkylene-, —C₂-C₈alkenylene-, —C₂-C₈alkynylene-, —[O(CR^{L2a}R^{L2b})ₘ₄]ₘ₅—, 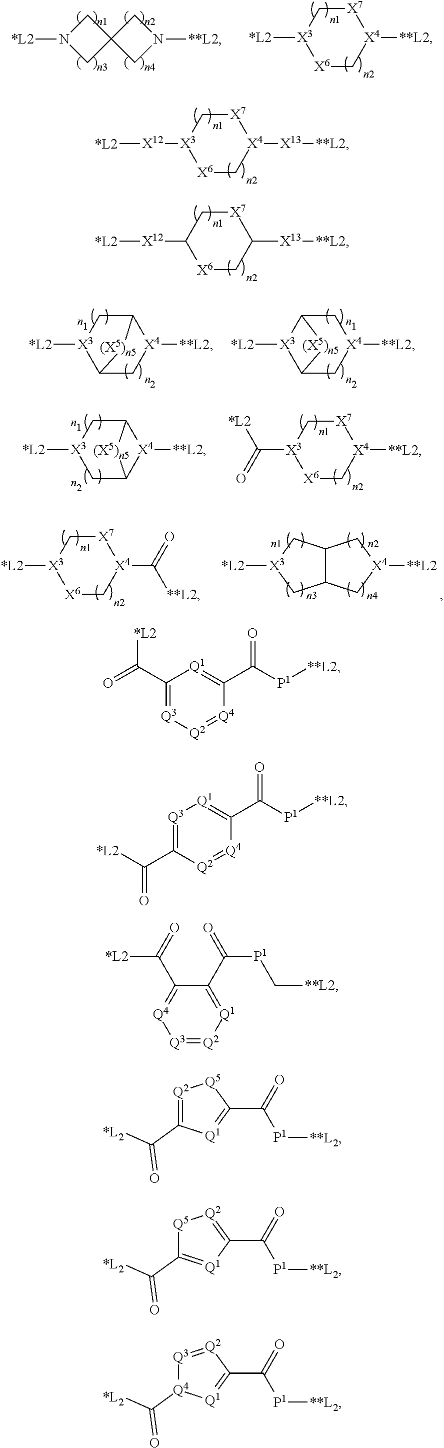 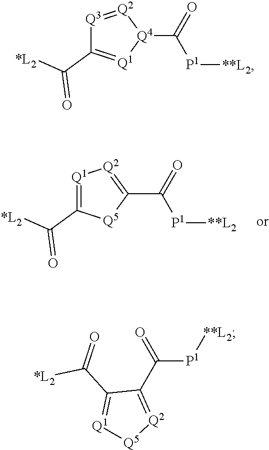
wherein each of said —C₃-C₈cycloalkylene-, *^{L2}—O—C₁-C₈alkylene-**^{L2}, *^{L2}—C₁-C₈alkylene-O—**^{L2}, *^{L2}SO₂—C₁-C₈alkylene-**^{L2}, *^{L2}—C₁-C₈alkylene-SO₂—**^{L2}, *^{L2}—C(O)—C₁-C₈alkylene-**^{L2}, *^{L2}—C₁-C₈alkylene- C(O)—**^{L2}, *^{L2}NR^{L2a}—C₁-C₈alkylene-**^{L2}, *^{L2}—C₁-C₈alkylene-NR^{L2a}—**^{L2}, —C₁-C₈alkylene-, —C₂-C₈alkenylene-, —C₂-C₈alkynylene-, 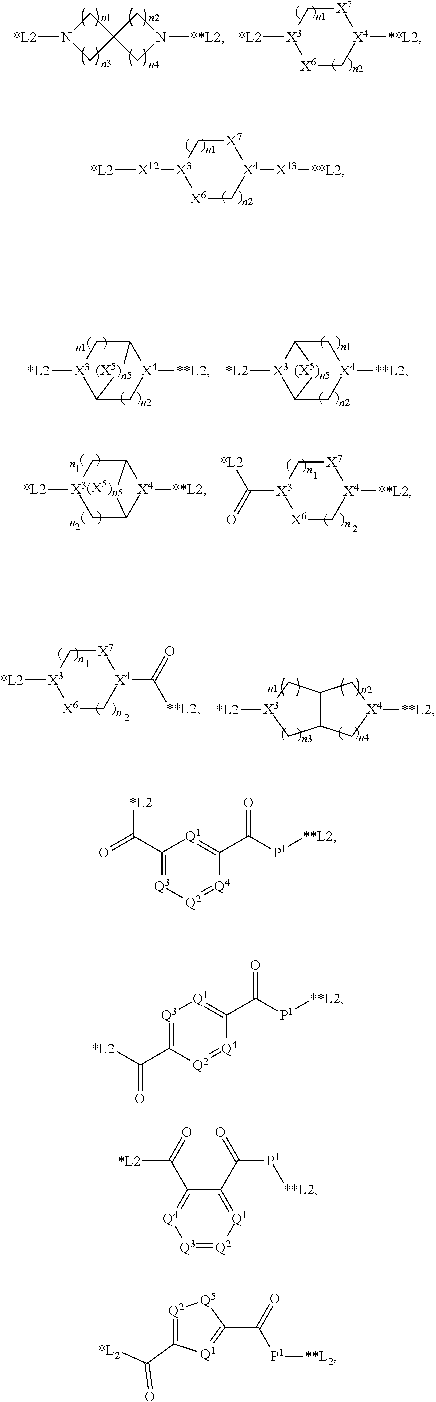 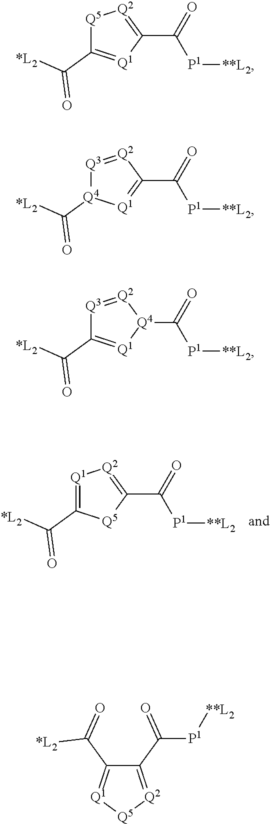
is optionally substituted with at least one substituent R^{L2c};
wherein *^{L2} refers to the position attached to 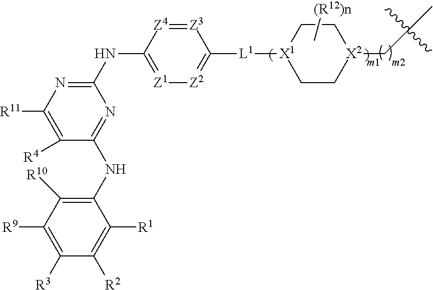
moiety, and **^{L2} refers to the position attached to the 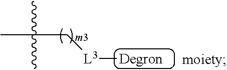
R^{L2a} and R^{L2b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L2d};
each of said R^{L2c} and R^{L2d} is independently selected from oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl;
L³ is selected from a single bond, —O—, —SO₂—, —C(O)—, —NR^{L3a}—, —C₃-C₈cycloalkylene-, *^{L3}—O—C₁-C₈alkylene-**^{L3}, *^{L3}—C₁-C₈alkylene-O—**^{L3}, *^{L3}—SO₂—C₁-C₈alkylene-**^{L3}, *^{L3}—C₁-C₈alkylene-SO₂—**^{L3}, *^{L3}—C(O)—C₁-C₈alkylene-**^{L3}, *^{L3}—C₁-C₈alkylene-C(O)—**^{L3}, *^{L3}—NR^{L3a}—C₁-C₈alkylene_**^{L3}, *^{L3}—C₁-C₈alkylene- NR^{L3a}—**^{L3}, *^{L3}—NR^{L3a}C(O)—**^{L3}, *^{L3}—C(O)NR^{L3a}—**^{L3}, C₁-C₈alkylene-, —C₂-C₈alkenylene-, —C₂-C₈alkynylene-, —[O(CR^{L3a}R^{L3b})ₘ₄]ₘ₅—, 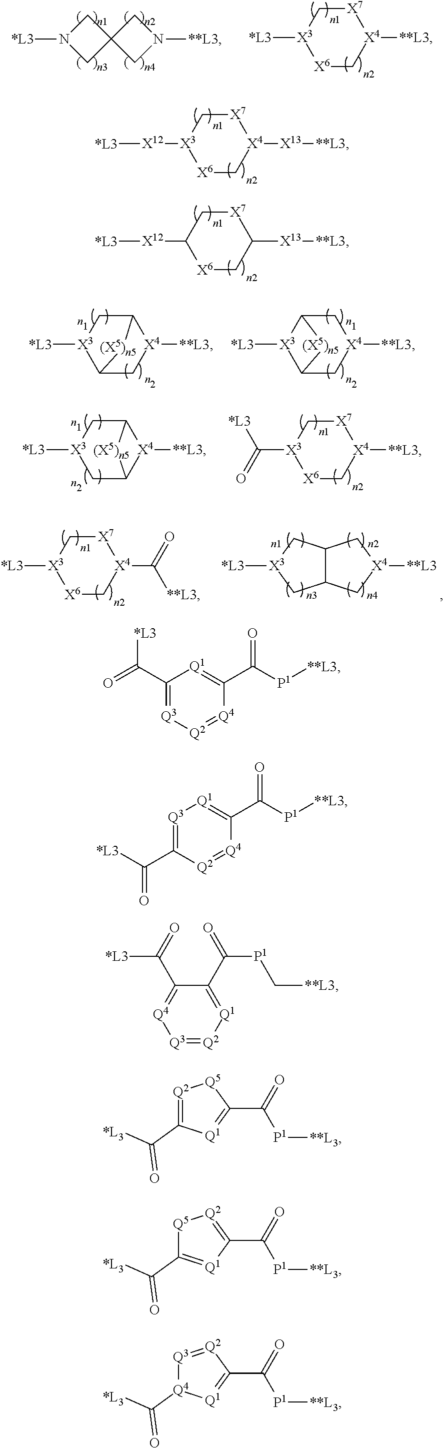 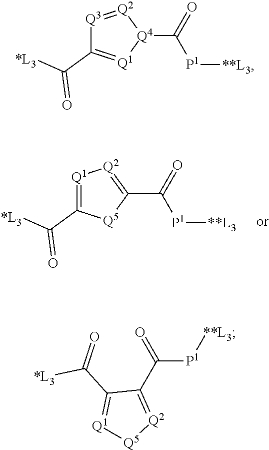
wherein each of said —C₃-C₈cycloalkylene-, *^{L3}—O—C₁-C₈alkylene-**^{L3}, *^{L3}—C₁-C₈alkylene-O—**^{L3}, *^{L3}—SO₂—C₁-C₈alkylene-**^{L3}, *^{L3}—C₁-C₈alkylene-SO₂**^{L3}, *^{L3}—C(O)—C₁-C₈alkylene**^{L3},*^{L3}—C₁-C₈alkylene- C(O)—**^{L3}, *^{L3}—NR^{L3a}—C₁-C₈alkylene-**^{L3}, *_{L3}—C₁-C₈alkylene-NR^{L3a_}**^{L3}, —C₁-C₈alkylene-, —C₂-C₈alkenylene-, —C₂-C₈alkynylene-, 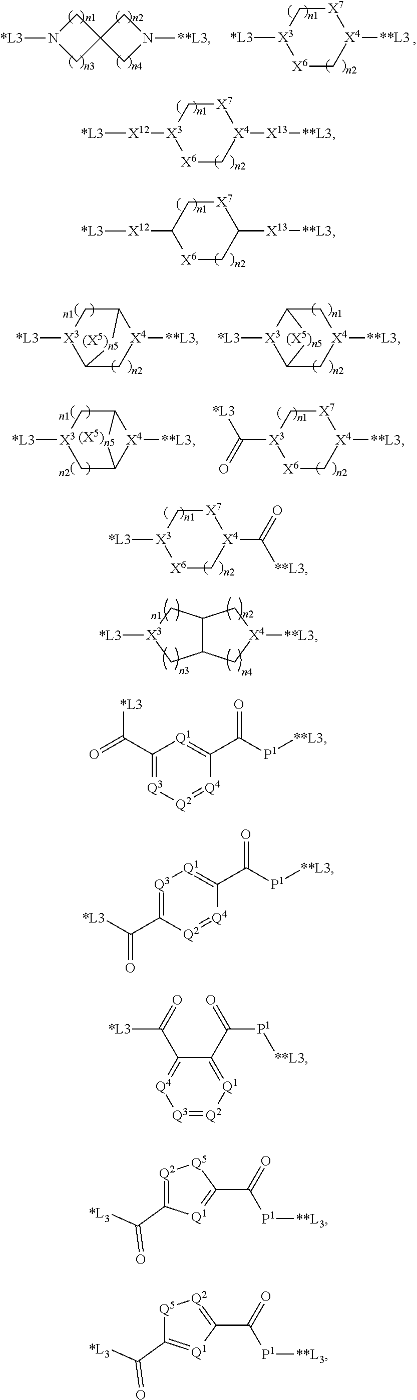 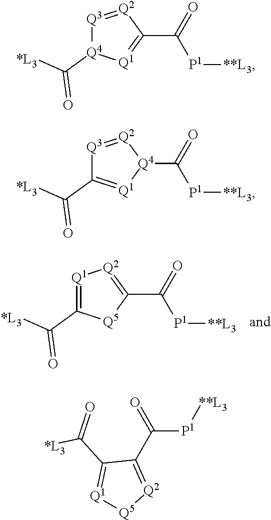
is optionally substituted with at least one substituent R^{L3c};
wherein *^{L3} refers to the position attached to 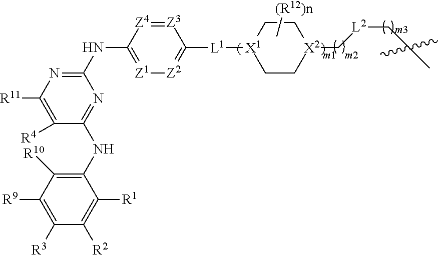
moiety, and **^{L3} refers to the position attached to the 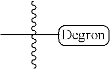
moiety;
R^{L3a} and R^{L3b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{L3d};
each of said R^{L3c} and R^{L3d} is independently selected from oxo (═O), halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl; or
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁-C₈alkyl; 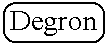
is selected from 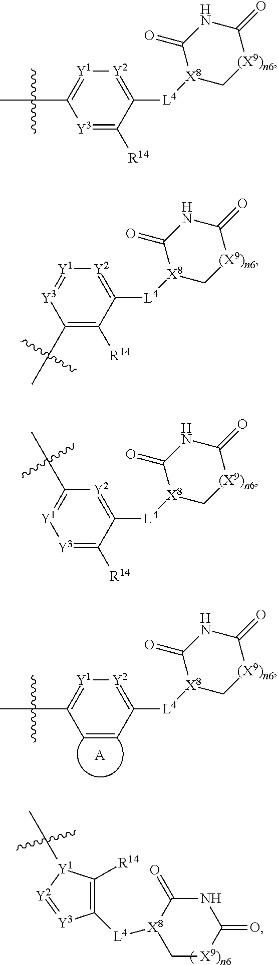 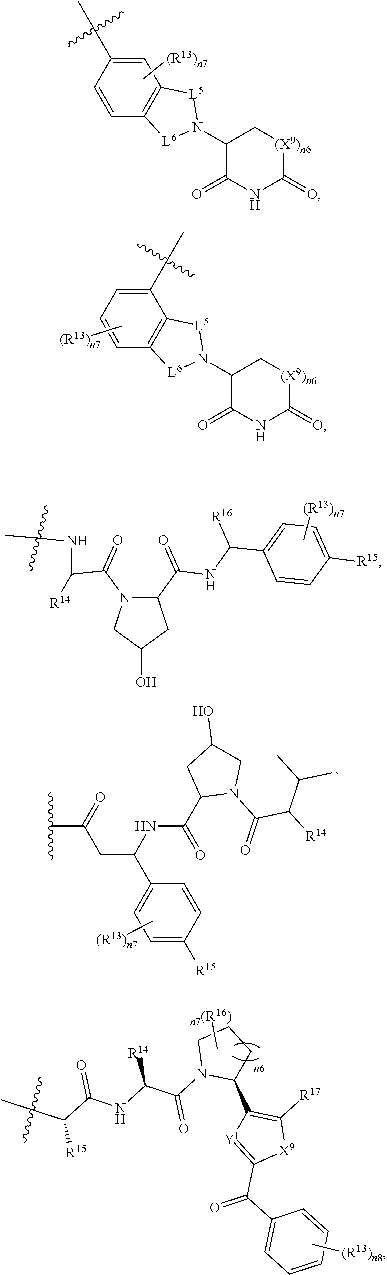 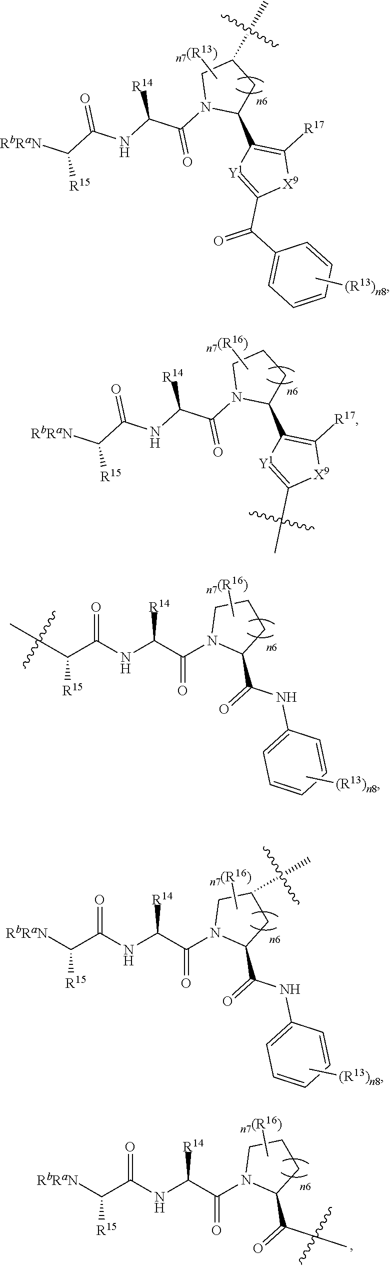 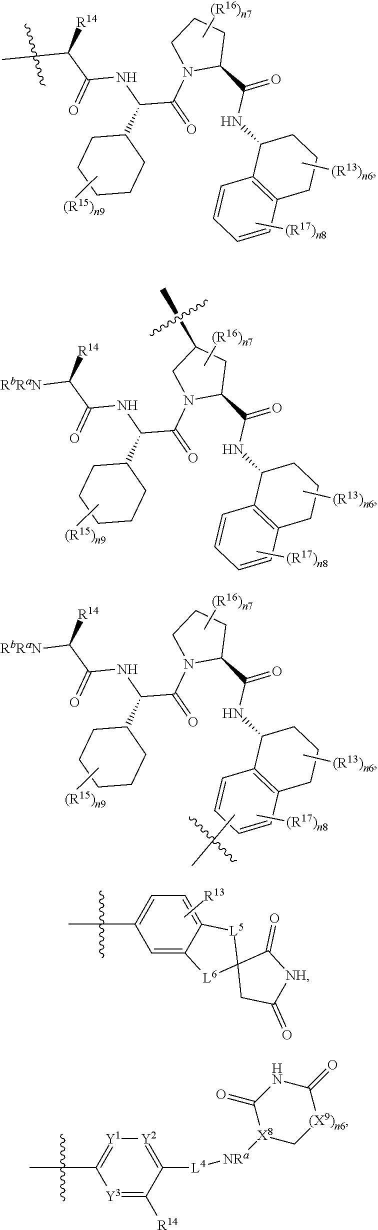 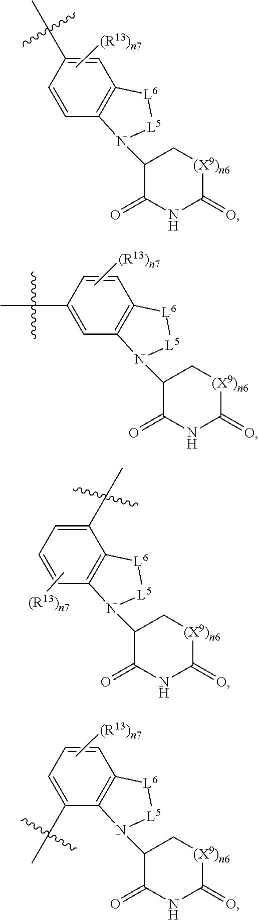 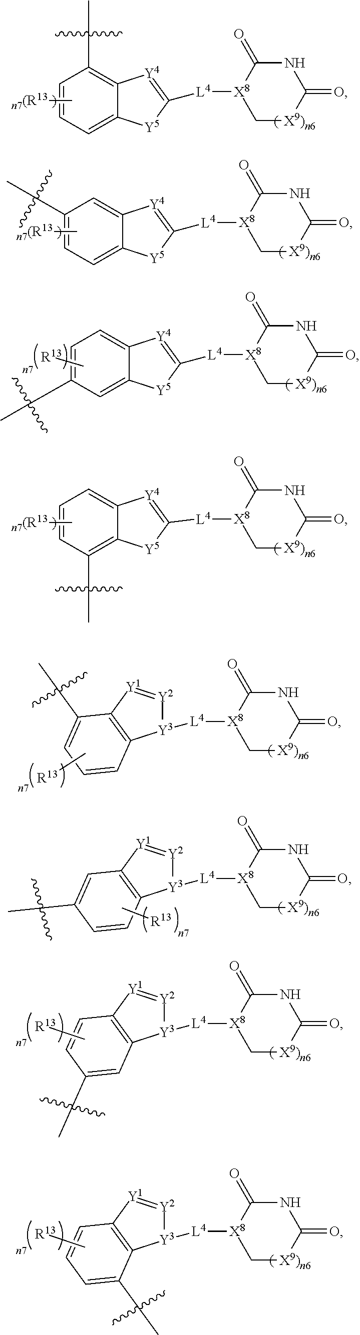 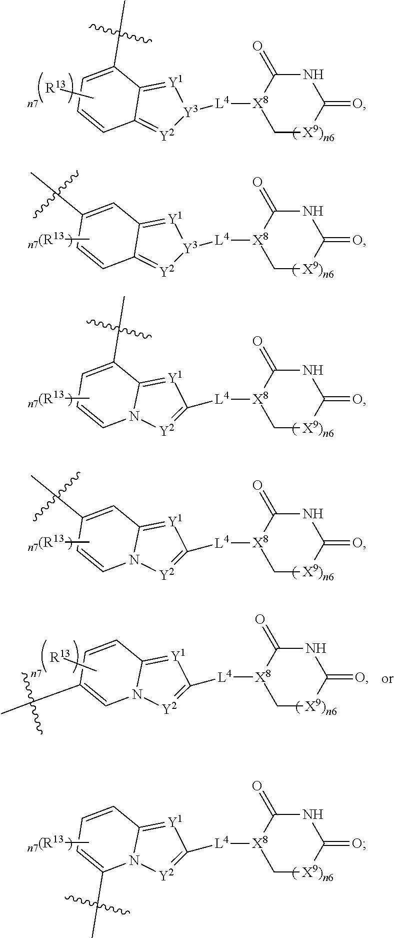
Ring A is selected from 3- to 12-membered cycloalkyl, 3- to 12-membered heterocyclyl, aryl, or heteroaryl;
R¹³, R¹⁴, R¹⁵, R¹⁶ and R¹⁷ are each independently selected from hydrogen, halogen, CN, —C₁-C₈alkyl, —C₁-C₈alkoxy, C₃-C₈cycloalkyl, 3- to 8-membered heterocyclyl, C₆-C₁₂aryl or 5- to 12-membered heteroaryl; said each —C₁-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent halogen, —C₁-C₈alkyl, C₁-C₈alkoxy-C₁-C₈alkyl-, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
X¹, X², X³, X⁴ and X⁸ are each independently selected from —CR^{a}, or N;
X⁵, X⁶, X⁷ and X⁹ are each independently selected from —NR^{a}—, —O—, —S— and —CR^{a}R^{b}—;
X¹² and X¹³ are each independently selected from —C(O)—, —NR^{a}— and —O—;
L⁴, L⁵ and L⁶ are each independently selected from a single bond, —O—, —NR^{a}—, —(CR^{a}R^{b})n₈-, —O(CR^{a}R^{b})n₈-, —NR^{a}(CR^{a}R^{b})n₈- or —C(O)—;
Q¹, Q², Q³, Q⁴, Y¹, Y², Y³ and Y⁴ are each independently selected from CR^{a} or N;
Y⁵ is selected from NR^{a}, 0 or S;
Q⁵ is each independently selected from —O—, —NR^{a}—, —CR^{a}R^{b}—, —S— or —C(O)—;
P¹ is a single bond, —O—, —NR^{a}—, —CR^{a}R^{b}—, —S—, —SO— or —SO₂—;
at each occurrence, R^{a} and R^{b} are each independently selected from hydrogen, hydroxy, halogen, CN, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl, each of said —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁-C₈alkyl, —C₂-C₈alkenyl, —C₂-C₈alkynyl, —C₁-C₈alkoxy, —C₂-C₈alkenyl, —C₂-C₈alkynyl, C₃-C₈cycloalkyl, 3- to 8-membered heterocyclyl, C₆-C₁₂aryl or 5- to 12-membered heteroaryl;
m₁ is 0, 1 or 2;
m₂ and m₃ are each independently 0, 1, 2, 3, 4, 5, 6, 7 or 8;
m₄ and m₅ are each independently 0, 1, 2 or 3;
n, n₁, n₂, n₃, n₄ and n₅ are each independently 0, 1, 2 or 3; and
n₆, n₇, n₈ and n₉ are each independently 0, 1, 2, 3 or 4.

2. The compound of claim 1, wherein the compound is selected from formula (II), (III), (IV), (V), (VI) or (VII), 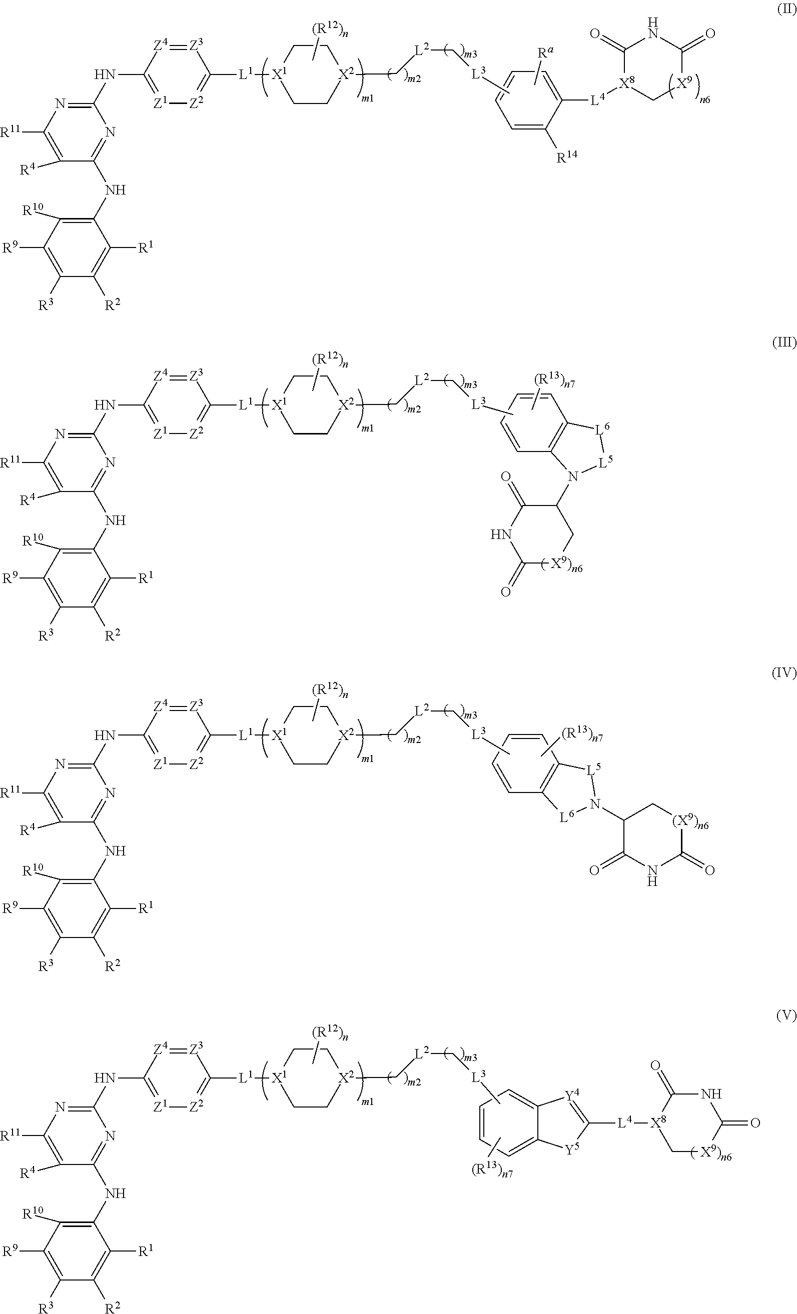 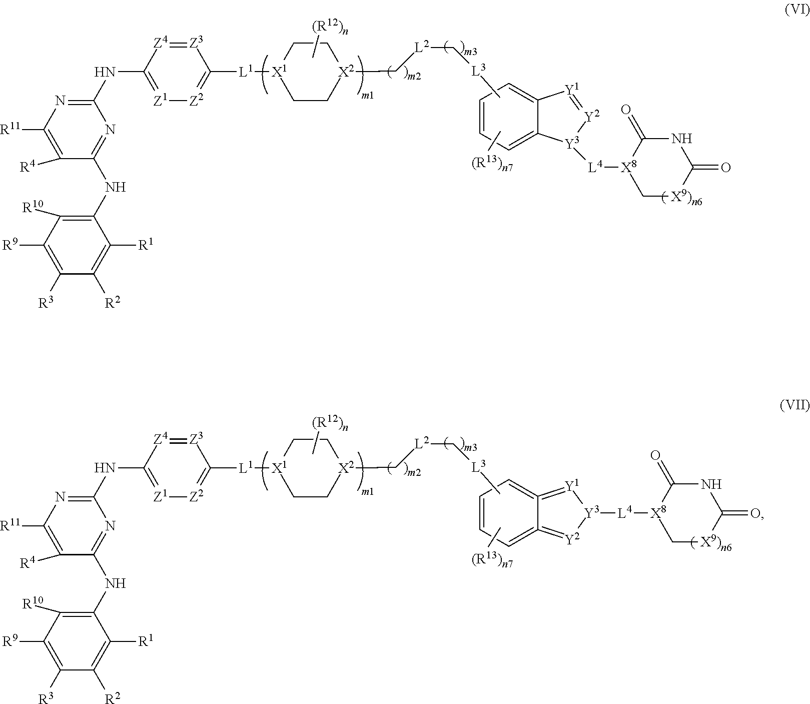
R¹, R², R³, R⁴, R⁹, R¹⁰, R¹¹, R¹², R¹³, R¹⁴, R^{a}, Z¹, Z², Z³, Z⁴, L¹, L², L³, L⁴, L⁵, L⁶, X¹, X², X⁸, X⁹, Y¹, Y², Y³, Y⁴, Y⁵, n, n6, n7, m1, m2 and m3 are each independently defined as claim 1.

3. The compound of claim 1-2, wherein R¹ is selected from —P(O)R^{1a}R^{1b} or —N(R^{1a})—SO₂R^{1b}, wherein R^{1a} and R^{1b} are each independently selected from hydrogen, halogen, —C₁-C₈alkyl (preferably —CH₃, —C₂H₅, —C₃H₇, —C₄H₉ or —C₅H₁₁; more preferably —CH₃, —CH₂CH₃, —CH₂CH₂CH₃,-iso-C₃H₇, —CH₂CH₂CH₂CH₃,-iso-C₄H₉, -sec-C₄H₉ or -tert-C₄H₉) or C₃-C₈cycloalkyl (preferably cyclopropyl, cyclobutyl or cyclopentyl).

4. The compound of any one of claims 1-3, wherein R₁ is selected from —P(O)(CH₃)₂, —NH—SO₂CH₃ or —N(CH₃)—SO₂CH₃.

5. The compound of any one of claims 1-4, wherein R₁ is —P(O)(CH₃)₂.

6. The compound of any one of claims 1-5, wherein R² and R³ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, —CN, —OR^{2a}, —SO₂R^{2a}, —SO₂NR^{2a}R^{2b}, —C(O)R^{2a}, —CO₂R^{2a}, —C(O)NR^{2a}R^{2b}, —NR^{2a}R^{2b}, —NR^{2a}COR^{2b}, —NR^{2a}CO₂R^{2b}, or —NR^{2a}SO₂R^{2b}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl is optionally substituted with at least one substituent R^{2a}, or
R² and R³ together with the carbon atoms to which they are attached, form a 5 or 6-membered unsaturated or saturated ring, said ring comprising 0, 1, 2 or 3 heteroatoms independently selected from nitrogen, oxygen or sulfur; said ring is optionally substituted with at least one substituent R^{2e};
R^{2e}, at each occurrence, is independently —H, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl, oxo (═O), —CN, CF₃, CHF₂, CH₂F, thioxo (═S), —SCF₃, —SCHF₂, —SCH₂F, —SCH₂CF₃, —SCF₂CH₃, —SCF₂CF₃, —SO₂R^{2a}, —SO₂NR^{2a}R^{2b}, —C(O)R^{2a}, —CO₂R^{2a}, —C(O)NR^{2a}R^{2b}, —NR^{2a}R^{2b}, —NR^{2a}COR^{2b}, —NR^{2a}CO₂R^{2b} or —NR^{2a}SO₂R^{2b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl is optionally substituted with at least one substituent R^{2a};
R^{2a} and R^{2b} are each independently selected from hydrogen, methyl, ethyl, propyl, butyl, pentyl, hexyl, heptyl, octyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{2d}, at each occurrence, is independently selected from halogen, —OH, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

7. The compound of any one of claims 1-6, wherein R² and R³ together with the carbon atoms to which they are attached, form a 5 or 6-membered unsaturated (preferred aromatic) or saturated ring, said ring comprising 1 or 2 nitrogen heteroatoms; said ring is optionally substituted with at least one substituent —H, —F, —Cl, —Br, —I, methyl, ethyl, propyl (n- or iso-), butyl (n-, iso- or t-), pentyl, methoxy, ethoxy, propoxy, butoxy, pentoxy, cyclopropyl, cyclobutyl, cyclopentyl, cyclohexyl, cycloheptyl, cyclooctyl, —CH₂OH, —SCH₃, —SC₂H₅, oxo (═O), thioxo (═S), —CF₃, —CHF₂, —CH₂F, —SCF₃, —OMe, —OC₂H₅, —CN, —C(O)CH₃,

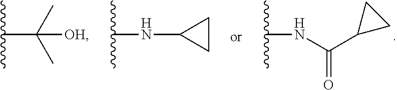

8. The compound of any one of claims 1-7, wherein R² and R³ together with the carbon atoms to which they are attached, form a 6-membered unsaturated (preferred aromatic) ring, said ring comprising 1 or 2 nitrogen heteroatoms; said ring is optionally substituted with one substituent —H, —F, —Cl, —Br, —I, methyl, ethyl or cyclopropyl.

9. The compound of any one of claims 1-8, wherein R⁴ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl, —C₂-C₈alkynyl or —C₁-C₈alkoxy; each of methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl or —C₂-C₈alkynyl is optionally substituted with —F, —Cl, —Br, —I, oxo (═O), or —CN.

10. The compound of any one of claims 1-9, wherein R⁴ is hydrogen, —F, —Cl, —Br, —I, —CH₃, —CF₃, —CH₂F, —CHF₂, —C(O)OMe, —C(O)OEt, —C(O)OⁱPr or —C(O)O^{t}Bu.

11. The compound of any one of claims 1-10, wherein R⁴ is hydrogen, —F, —Cl, —Br or —I.

12. The compound of any one of claims 1-11, wherein R⁹, R¹⁰, R¹¹ and R¹² are each independently selected from hydrogen, —F, —Cl, —Br, —I, methyl, ethyl, propyl, butyl, pentyl, hexyl, heptyl, octyl, —NR^{9a}R^{9b}, —OR^{9a}, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo (═O), or —CN; each of -methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9c};
R^{9a} and R^{9b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9d};
R^{9c} and R^{9d} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

13. The compound of any one of claims 1-12, wherein R⁹, R¹¹, R¹¹ and R¹² are each independently selected from hydrogen, —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, cyclohexyl, —CH₂OH, —CH₂OMe, oxo (═O), or —CN.

14. The compound of any one of claims 1-13, wherein R⁹, R¹⁰, R¹¹ and R¹² are each independently selected from hydrogen, —CH₃, —F, —Cl, —Br or —I.

15. The compound of any one of claims 1-11, wherein two R¹² together with the carbon atoms to which they are attached, form a 3, 4, 5, 6, 7 or 8-membered ring, said ring comprising 0, 1, 2 or 3 heteroatoms independently selected from nitrogen, oxygen or sulfur; said ring is optionally substituted with at least one substituent R^{9C};
R^{9c} is independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

16. The compound of any one of claims 1-11, wherein two R¹² together with the carbon atoms to which they are attached, form a 3, 4, 5, 6, 7 or 8-membered ring, preferably form a 3, 4, 5 or 6-membered ring, said ring comprising 0, 1, 2 or 3 heteroatoms independently selected from nitrogen, oxygen or sulfur; said ring is optionally substituted with at least one substituent —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, or cyclohexyl.

17. The compound of any one of claims 1-5, wherein the 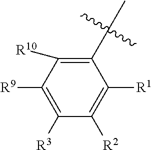
moiety is 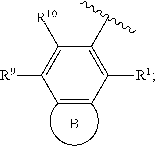
Ring B is a 5 or 6-membered unsaturated or saturated ring, said ring comprising 0, 1, 2 or 3 heteroatoms; said heteroatoms are independently selected from N, NR^{2e}, O or S;
said ring is optionally substituted with at least one substituent R^{2e}.

18. The compound of any one of claims 1-17, wherein the 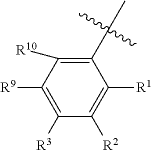
moiety is selected from 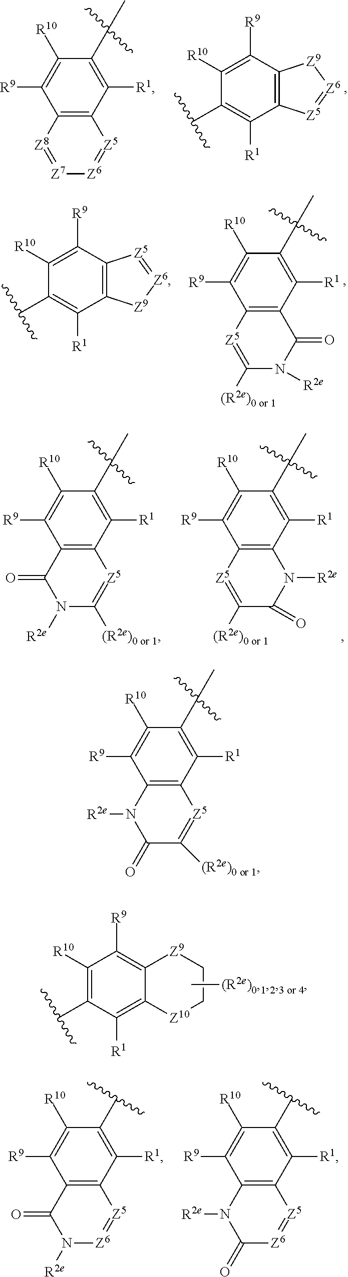 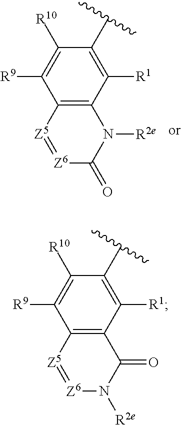
Z⁵, Z⁶, Z⁷ and Z⁸ are each independently selected from N, CH or CR^{2e};
Z⁹ and Z¹⁰ are each independently selected from O, S, NH or NR^{2e}.

19. The compound of any one of claims 1-18, wherein the 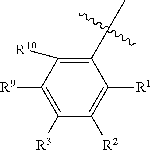
moiety is selected from

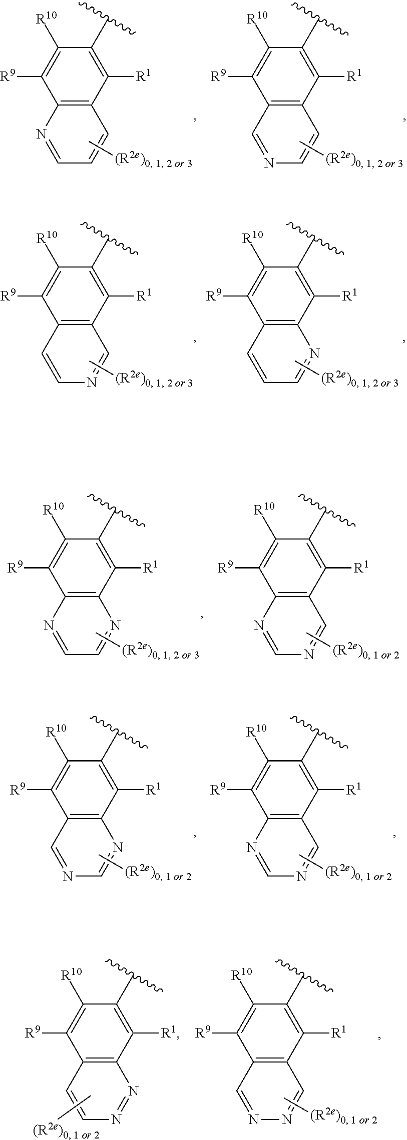

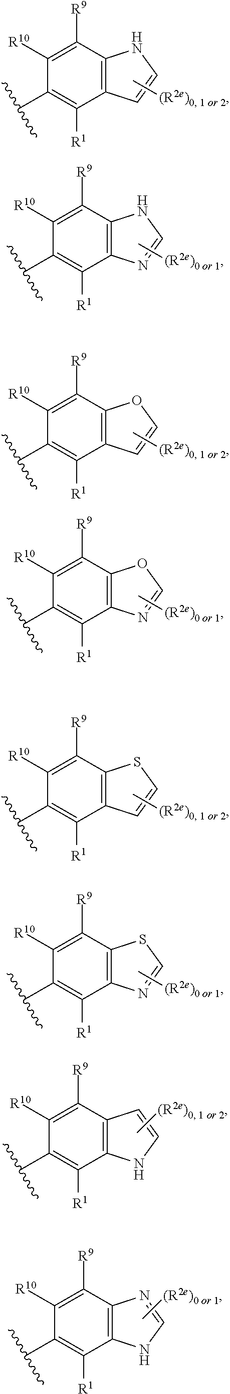

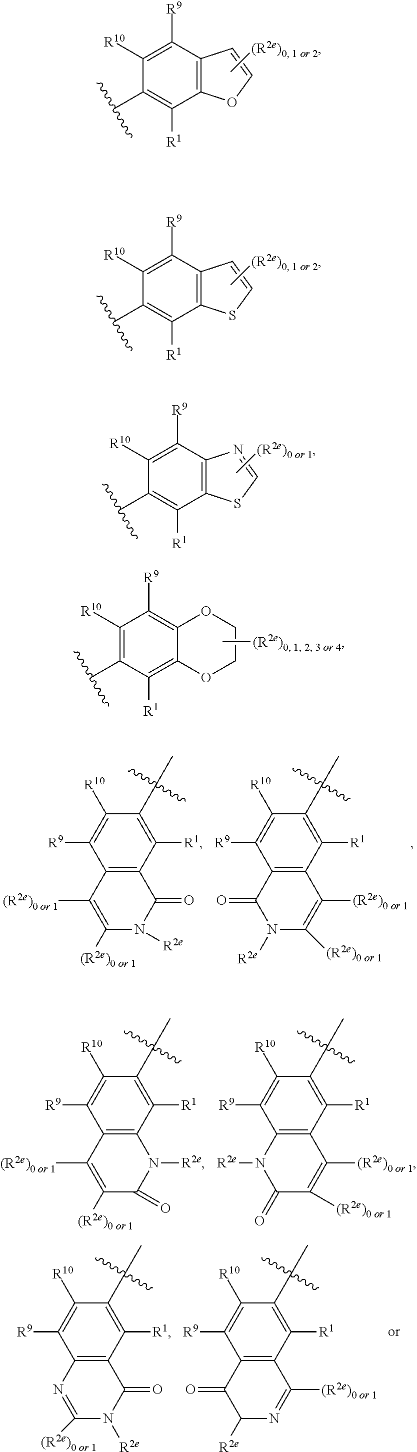

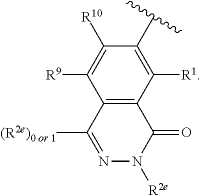

20. The compound of any one of claims 1-19, wherein the 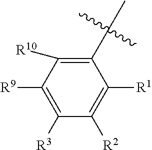
moiety is selected from

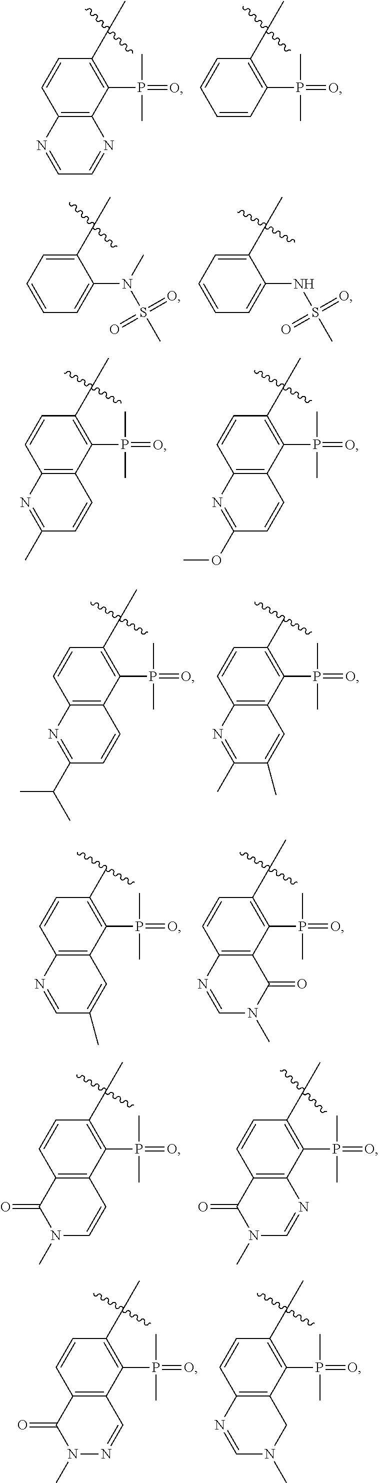

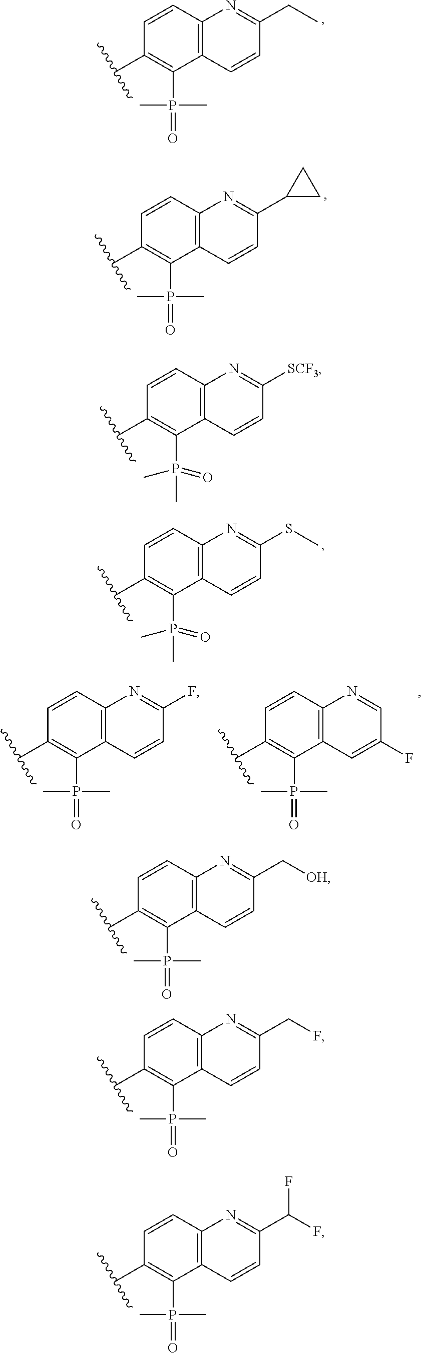

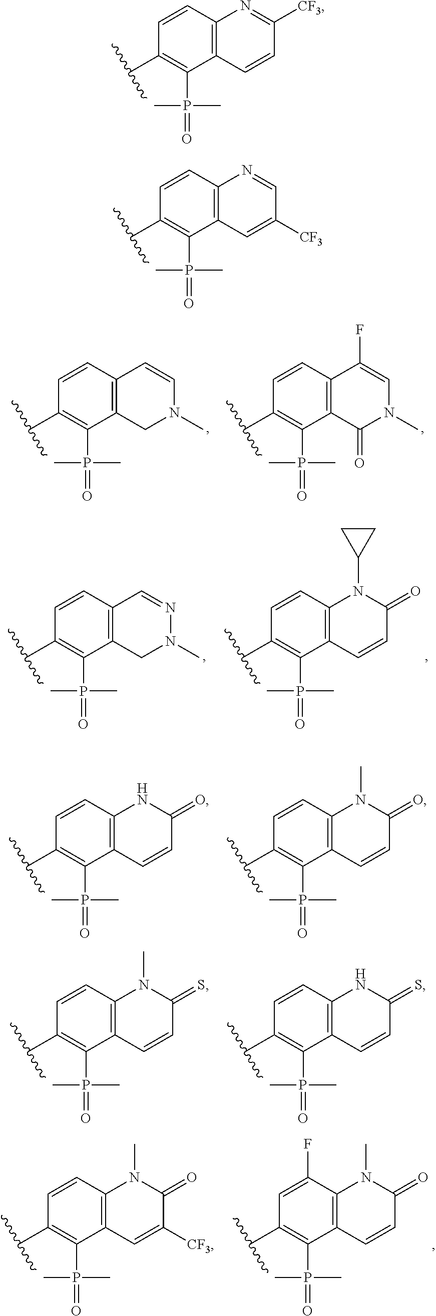

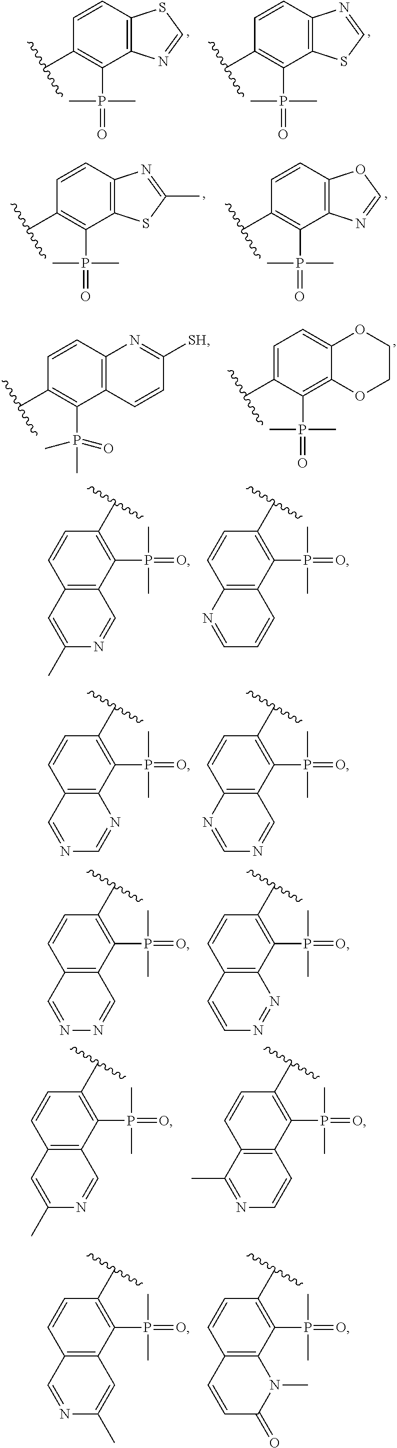

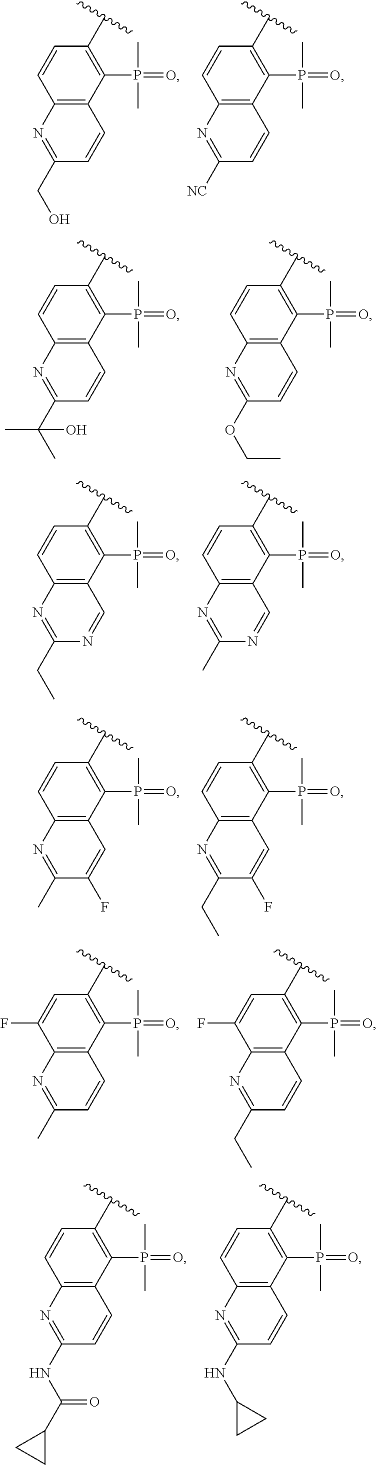

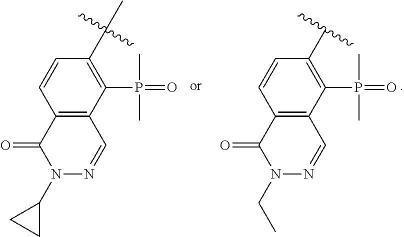 [SMILES: *c1ccc2c(=O)n(C3CC3)ncc2c1P(C)(C)=O ; *c1ccc2c(=O)n(CC)ncc2c1P(C)(C)=O]

21. The compound of any one of claims 1-20, wherein L¹ is selected from a single bond, —C₁-C₈alkylene-(preferably —CH₂—, —C₂H₄—, —C₃H₆—), —C(O)—C₁-C₈alkylene-(preferably —C(O)—CH₂—, —C(O)—C₂H₄—, —C(O)—C₃H₆—), —C₁-C₈alkylene-C(O)— (preferably —CH₂C(O)—, —C₂H₄—C(O)—, —C₃H₆—C(O)—), —C(O)—, —O—, —N(CH₃)—, —NH—, 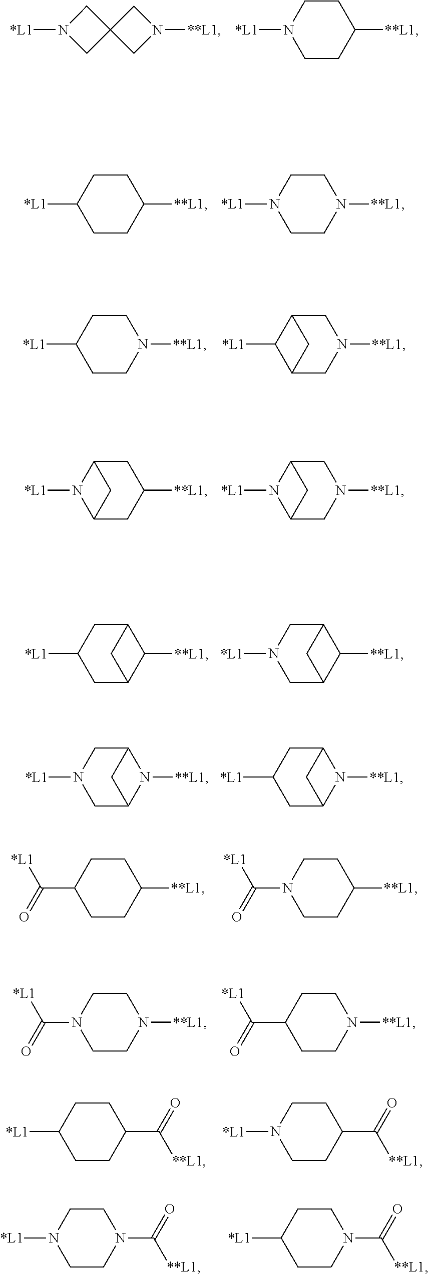 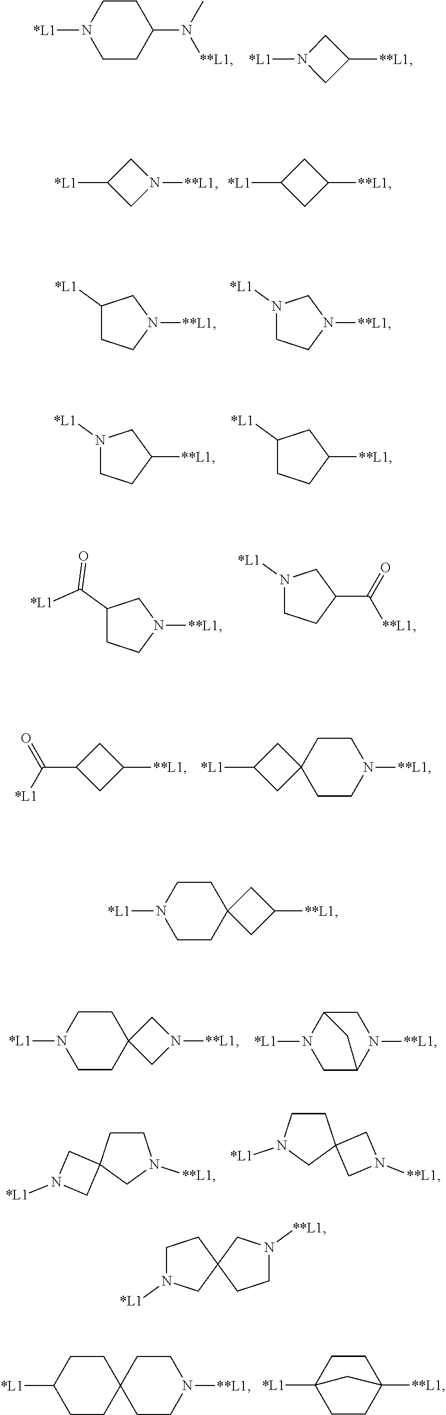 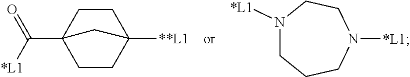
wherein each of said C₁-C₈alkylene-(preferably —CH₂—, —C₂H₄—, —C₃H₆—), *^{L1}—C(O)C₁-C₈alkylene_**^{L1} (preferably *^{L1}—C(O)—CH₂—**^{L1}, *^{L1}—C(O)—C₂H₄—**^{L1}, *^{L1}—C(O)—C₃H₆—**^{L1}), *^{L1}—C₁-C₈alkylene-C(O)—**^{L1} (preferably *^{L1}—CH₂—C(O)—**^{L1}, *^{L1}—C₂H₄—C(O)—**^{L1}, *^{L1}—C₃H₆—C(O)—**^{L}), —N(CH₃)—, —NH— 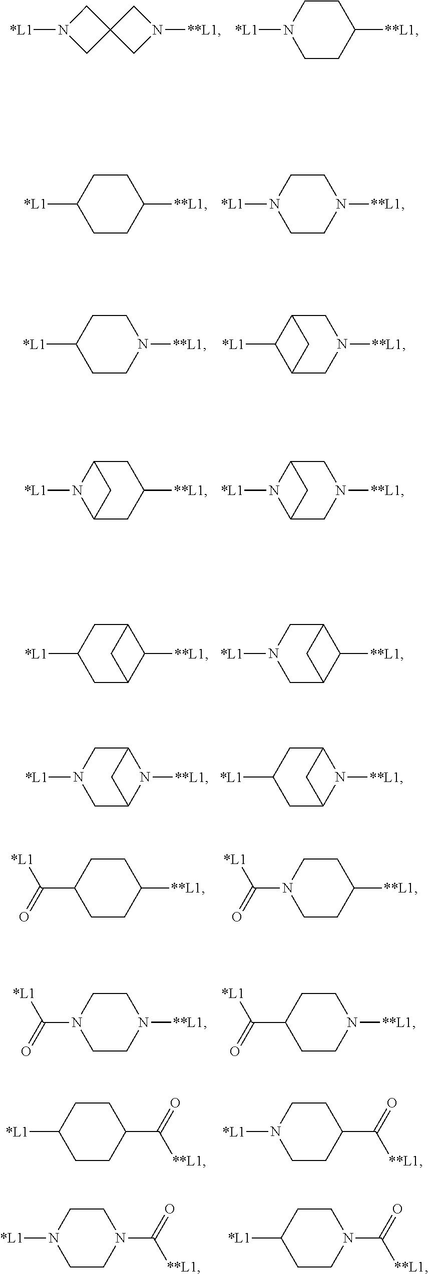 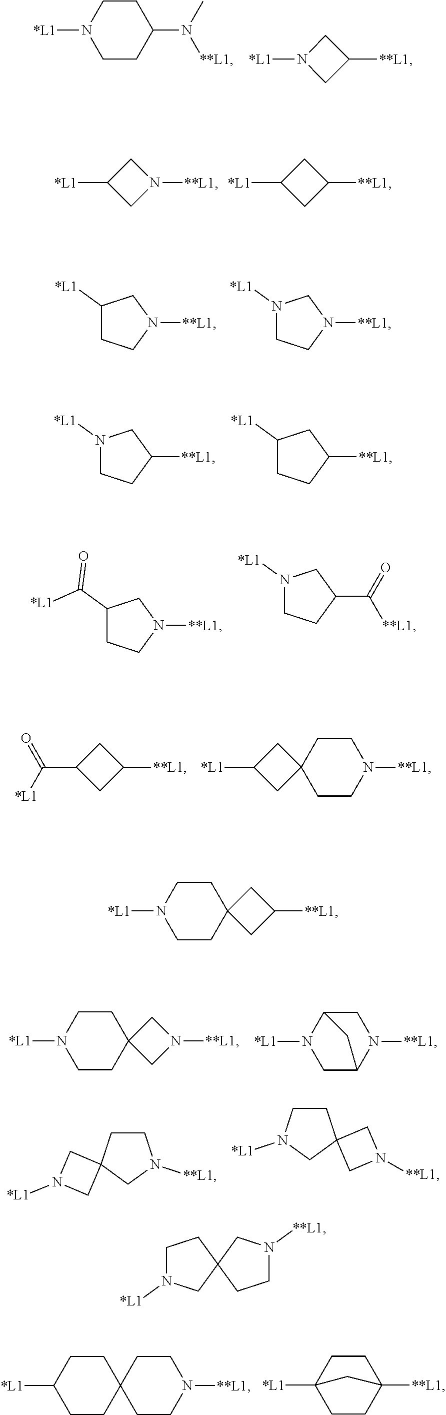 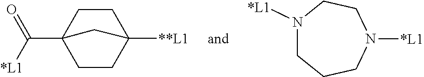
is optionally substituted with at least one R^{L1C};
each of said R^{L1c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L1c} together with the carbon atoms to which they are attached, form a 3-, 4-, 5-, 6- , 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl or octyl.

22. The compound of any one of claims 1-21, wherein L¹ is selected from a single bond, —C₁-C₈alkylene-(preferably —CH₂—, —C₂H₄—, —C₃H₆—), —C(O)—, —O—, —N(CH₃)—, —NH—,

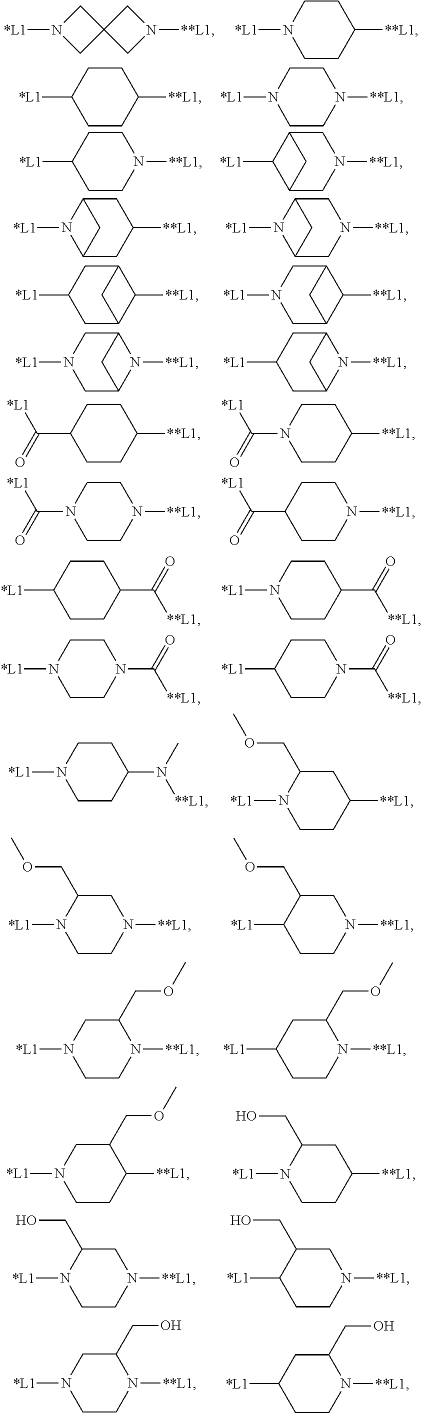

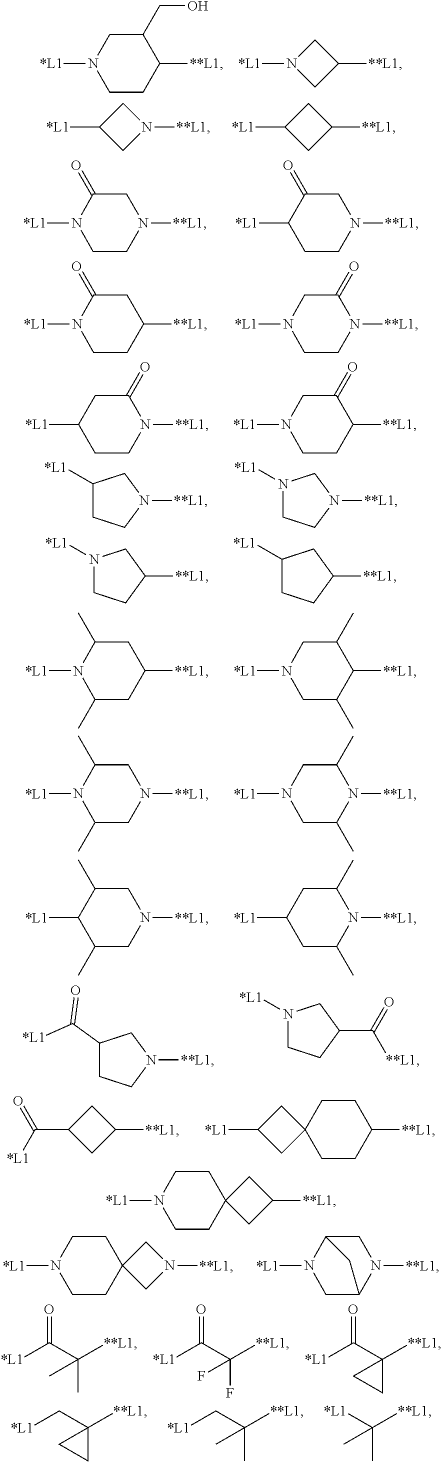

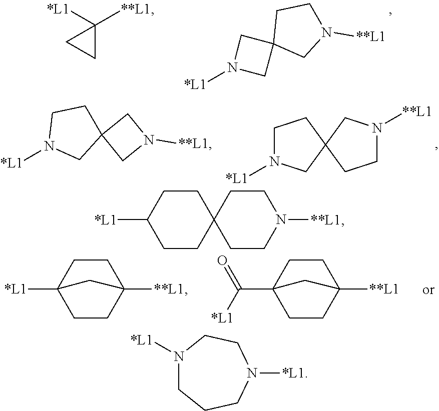

23. The compound of any one of claims 1-22, wherein X¹ and X² are each independently selected from —CR^{a} or N;
R^{a} is selected from hydrogen, —F, —Cl, —Br, —I, CN, methyl, ethyl, methoxy, ethoxy, cyclopropyl, each of said methyl, ethyl, methoxy, ethoxy, cyclopropyl, is optionally substituted with at least one substituent —F, —Cl, —Br, —I, hydroxy, methyl, ethyl, (preferably, X¹ and X² are each independently selected from CH, C(F), C(CH₃) or N);
m1=1 or 0;
R¹² is hydrogen, oxo (═O), methoxymethyl, hydroxymethyl, —CN or —CH₃.

24. The compound of any one of claims 1-23, wherein m1 is 1; preferably, 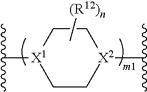
moiety is 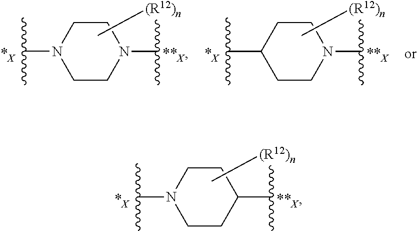
wherein *^{X} refers to the position attached to 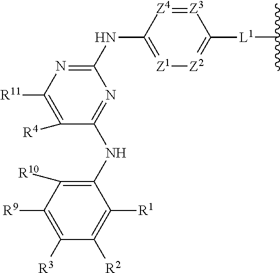
moiety, and **^{X} refers to the position attached to the 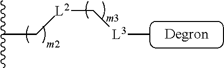
moiety.

25. The compound of any one of claims 1-24, wherein m1 is 1 preferably, 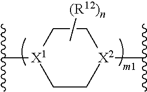
moiety is 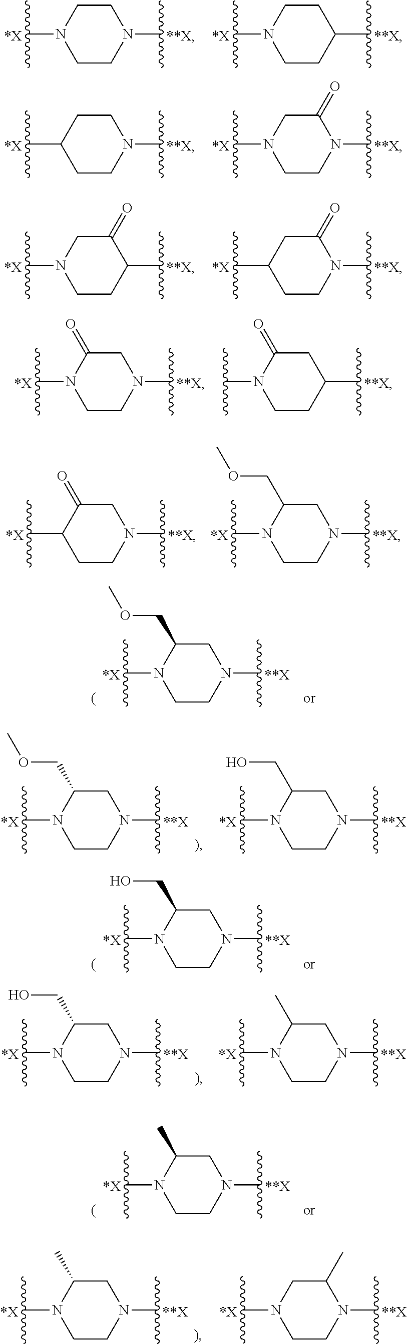 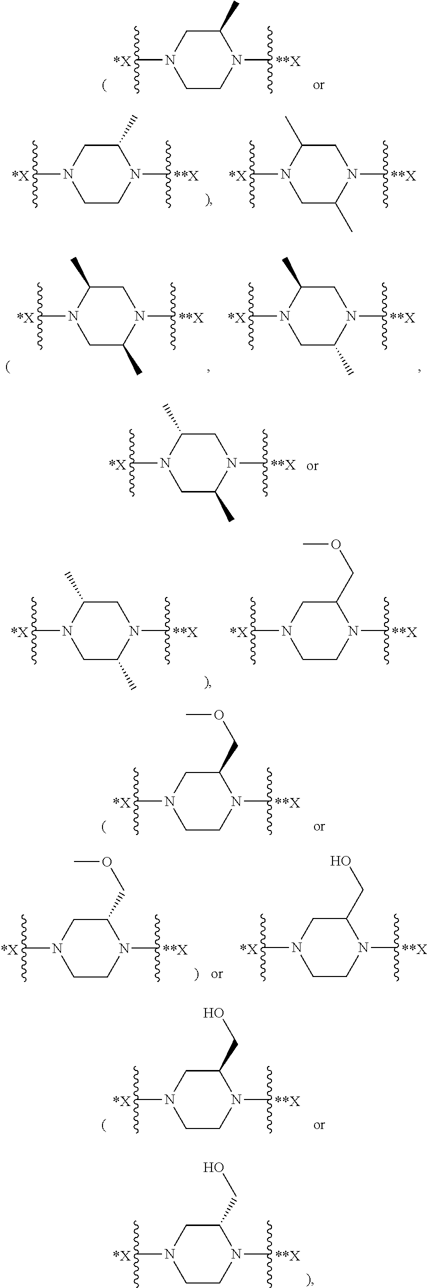
wherein *^{X} refers to the position attached to 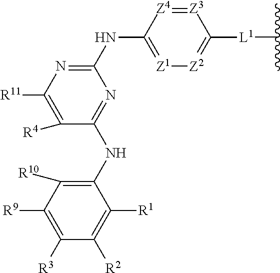
moiety, and **^{X} refers to the position attached to the 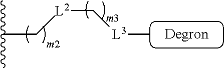
moiety.

26. The compound of any one of claims 1-25, wherein m1 is 1, 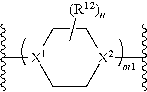
moiety is 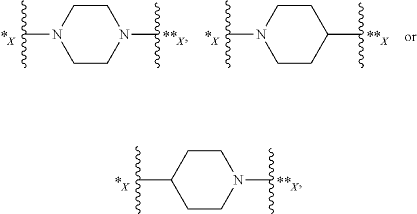 [SMILES: *N1CCN(*)CC1 ; *C1CCN(*)CC1 ; *C1CCN(*)CC1]
wherein *^{X} refers to the position attached to 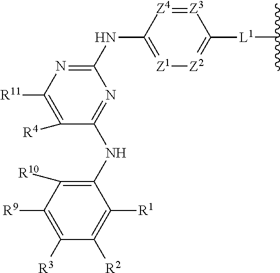
moiety, and **^{X} refers to the position attached to the 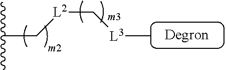
moiety.

27. The compound of any one of claims 1-26, wherein L² is selected from a single bond, —C₁-C₈alkylene-(preferably —CH₂—, —C₂H₄—, —C₃H₆—), *^{L2}—C(O)—C₁-C₈alkylene_**^{L2} (preferably *^{L2}—C(O)—CH₂—**^{L2}, *^{L2}—C(O)—C₂H₄—**^{L2}, *^{L2}—C(O)—C₃H₆—**^{L2}), *^{L2}—C₁-C₈alkylene-C(O)—**^{L2} (preferably *^{L2}—CH₂—C(O)—**^{L2}, *^{L2}—C₂H₄—C(O)—**^{L2}, *^{L2}—C₃H₆—C(O)—**^{L2}), —C(O)—, —O—, —N(CH₃)—, —NH—, 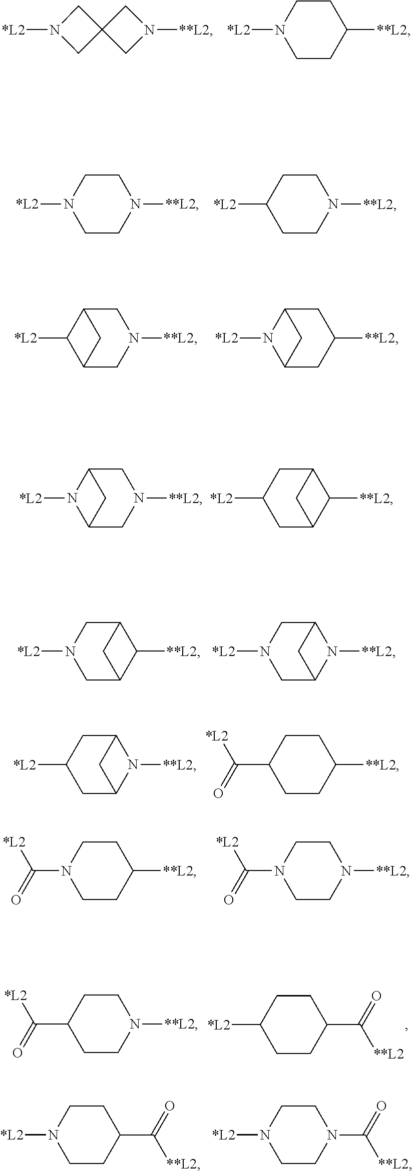 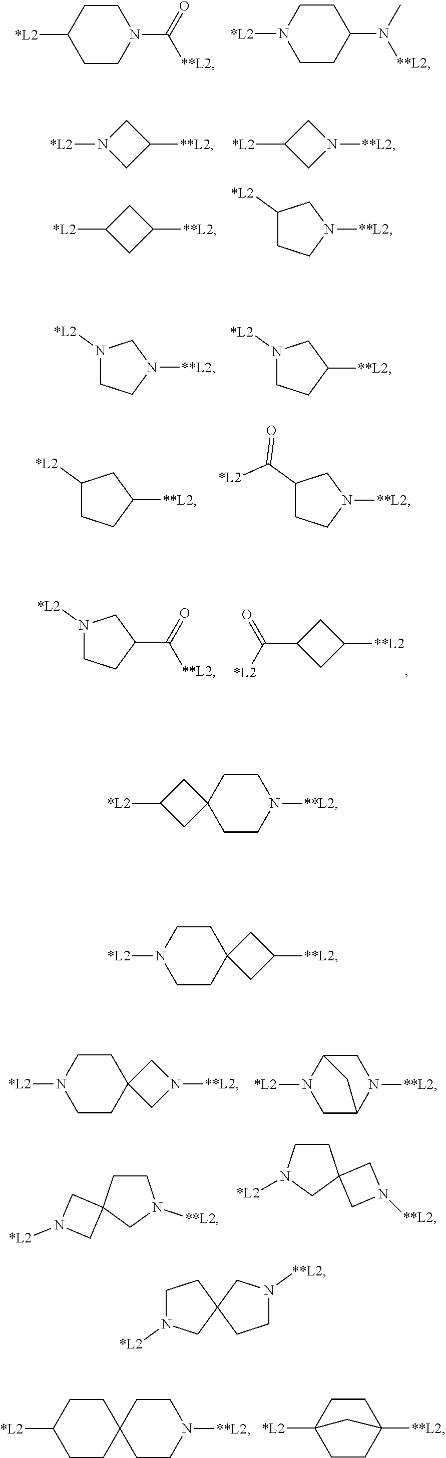 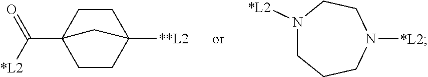
wherein each of said —C₁-C₈alkylene-(preferably —CH₂—, —C₂H₄—, —C₃H₆—), *^{L2}—C(O)—C₁-C₈alkylene-**^{L2} (preferably *^{L2}—C(O)—CH₂—**^{L2}, *^{L2}—C(O)—C₂H₄—**^{L2}, *^{L2}—C(O)—C₃H₆—**^{L2}), *^{L2}—C₁-C₈alkylene-C(O)—**^{L2} (preferably *^{L2}—CH₂—C(O)—**^{L2}, *^{L2}—C₂H₄—C(O)—**^{L2}, *^{L2}—C₃H₆—C(O)—**^{L2}), —N(CH₃)—, —NH— 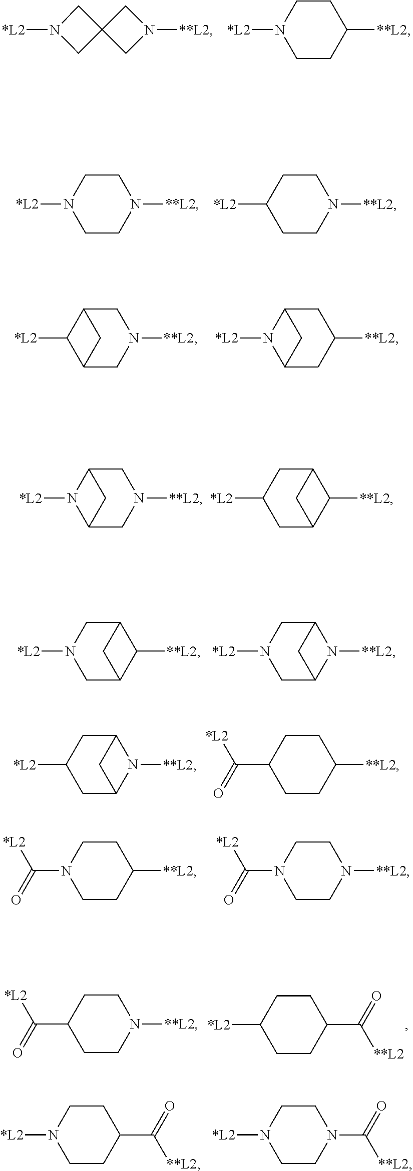 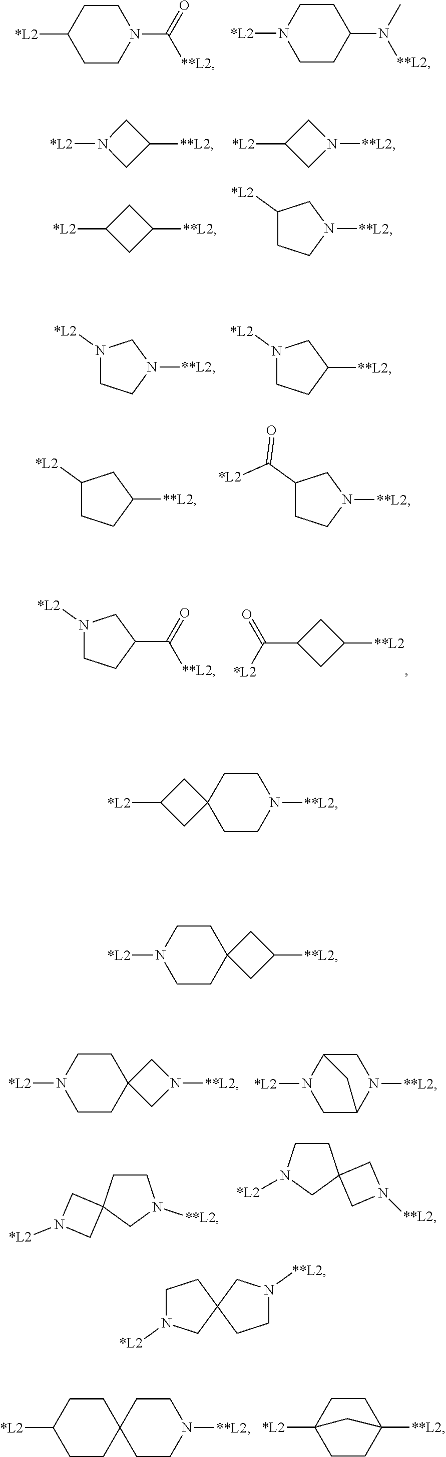 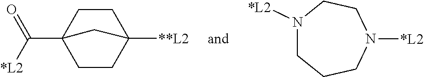
is optionally substituted with at least one R^{L2c};
each of said R^{L2c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L2c} together with the carbon atoms to which they are attached, form a 3-, 4-, 5-, 6- , 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl or octyl.

28. The compound of any one of claims 1-27, wherein L² is selected from a single bond, —C₁-C₈alkylene-(preferably —CH₂—, —C₂H₄—, —C₃H₆—), —C(O)—, —O—, —N(CH₃)—, —NH—,

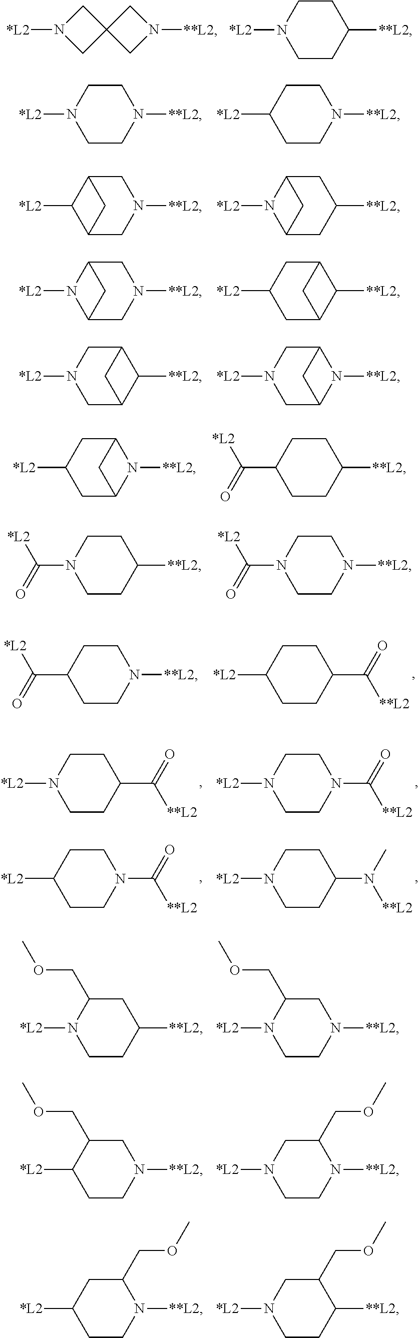

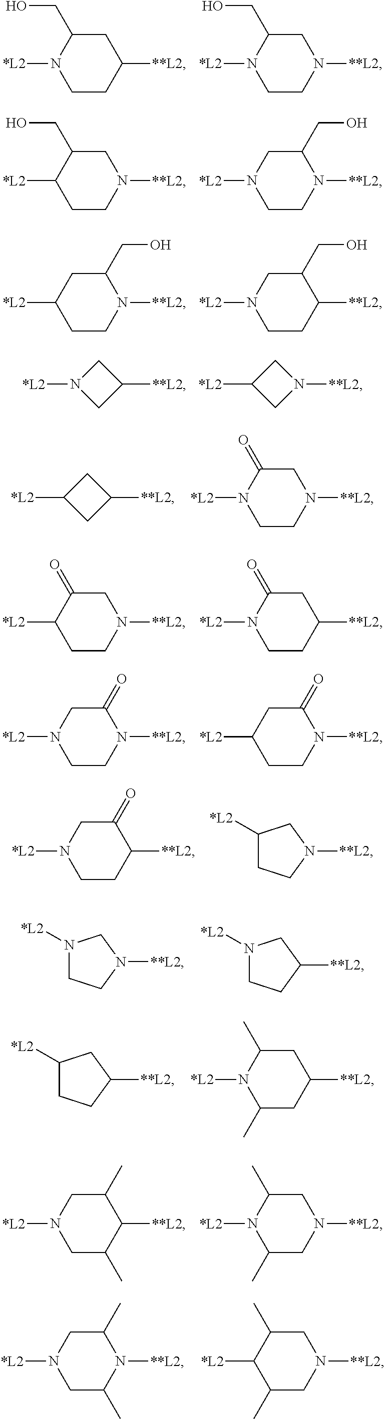

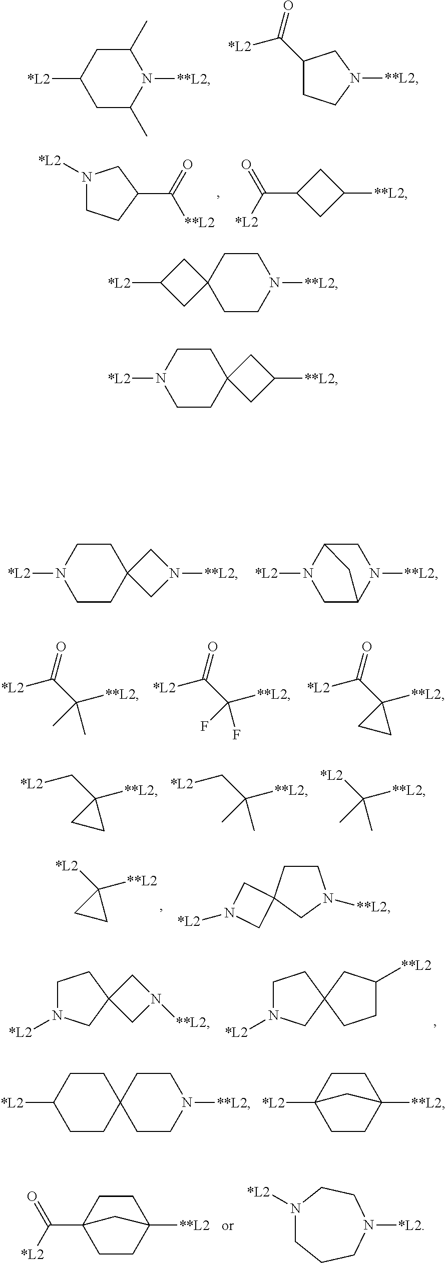

29. The compound of any one of claims 1-28, wherein L³ is selected from single bond, —C₁-C₈alkylene-(preferably —CH₂—, —C₂H₄—, —C₃H₆—), *^{L3}—C(O)—C₁-C₈alkylene-**^{L3} (preferably *^{L3}—C(O)—CH₂—**^{L3}, *^{L3}—C(O)—C₂H₄—**^{L3}, *^{L3}—C(O)—C₃H₆—**L¹³), *^{L3}—C₁-C₈alkylene-C(O)—**^{L3} (preferably *^{L3}—CH₂—C(Q)-**^{L3}, *^{L3}—C₂H₄—C(O)—**^{L3}, *^{L3}—C₃H₆—C(O)—**^{L3}), —C(O)—, —O—, —N(CH₃)—, —NH—, 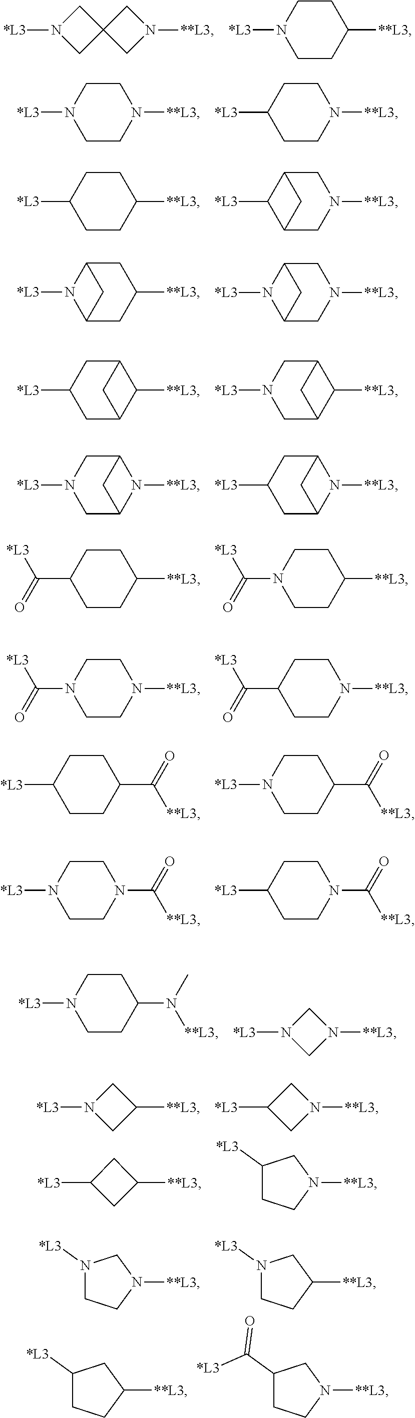 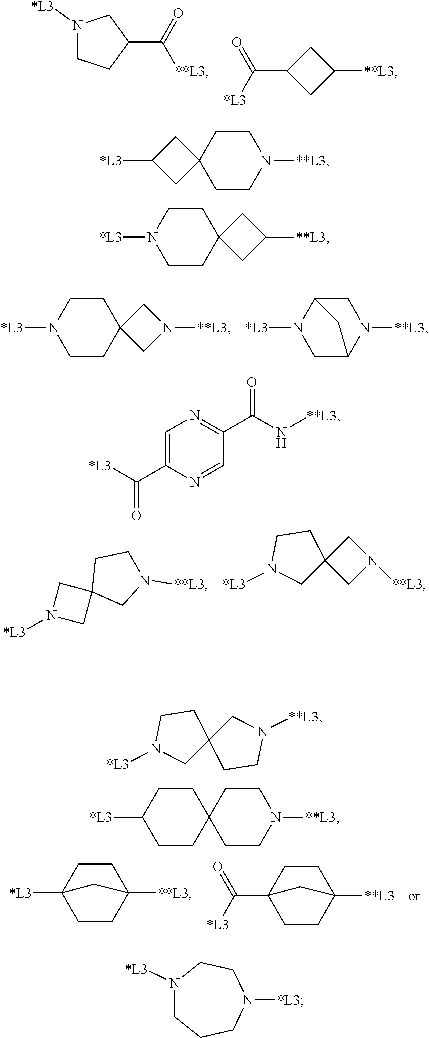
wherein each of said —C₁-C₈alkylene-(preferably —CH₂—, —C₂H₄—, —C₃H₆—), *^{L3}—C(O)—C₁-C₈alkylene_**^{L3} (preferably *^{L3}—C(O)—CH₂—**^{L3}, *^{L3}—C(O)—C₂H₄—**^{L3}, *^{L3}—C(O)—C₃H₆—**^{L3}), *^{L3}—C₁-C₈alkylene-C(O)—**^{L3} (preferably *^{L3}—CH₂—C(O)—**^{L3}, *^{L3}—C₂H₄—C(O)—**^{L3}, *^{L3}—C₃H₆—C(O)—**^{L3}), —N(CH₃)—, —NH—, 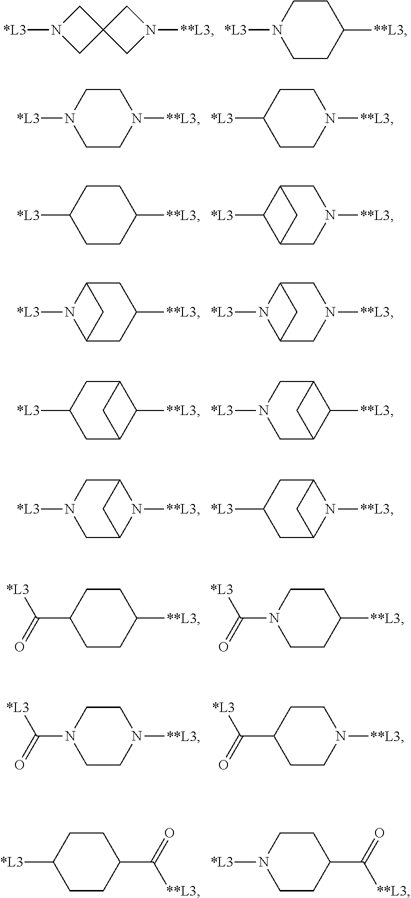 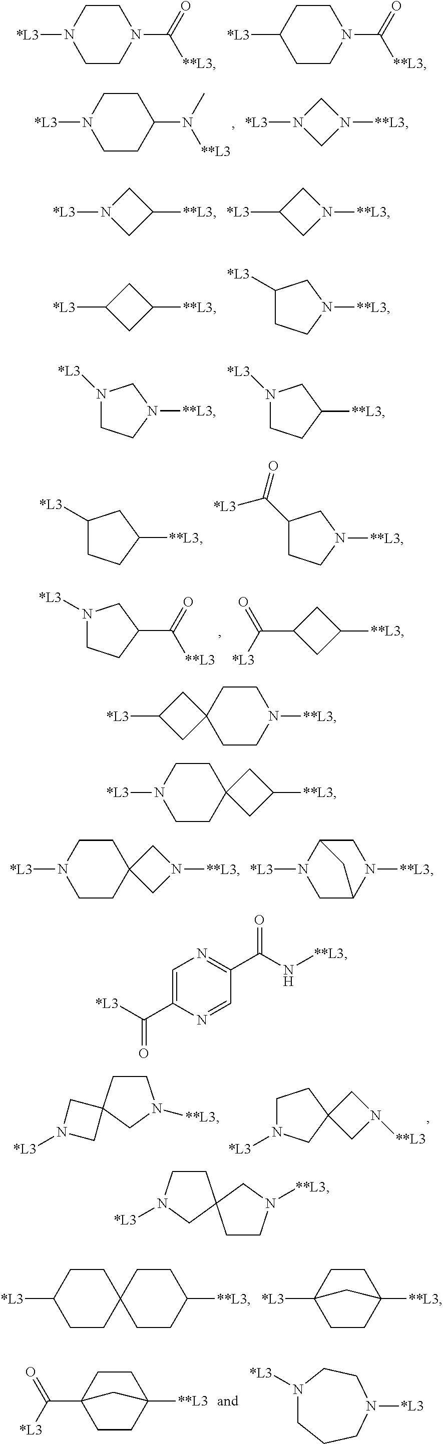
is optionally substituted with at least one R^{L3c};
each of said R^{L3c} is independently oxo (═O), F, Cl, Br, I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl; or
two R^{L3c} together with the carbon atoms to which they are attached, form a 3-, 4-, 5-, 6- , 7- or 8-membered ring, said ring comprising 0, 1, 2 or 3 heteroatoms independently selected from nitrogen, oxygen or sulfur; said ring is optionally substituted with at least one substituent F, Cl, Br, I, hydroxy, methyl, ethyl, propyl, butyl, pentyl, hexyl, heptyl, octyl.

30. The compound of any one of claims 1-29, wherein L³ is selected from a single bond —C₁-C₈alkylene-(preferably —CH₂—, —C₂H₄—, —C₃H₆—), —C(O)—, —O—, —N(CH₃)—, —NH—,

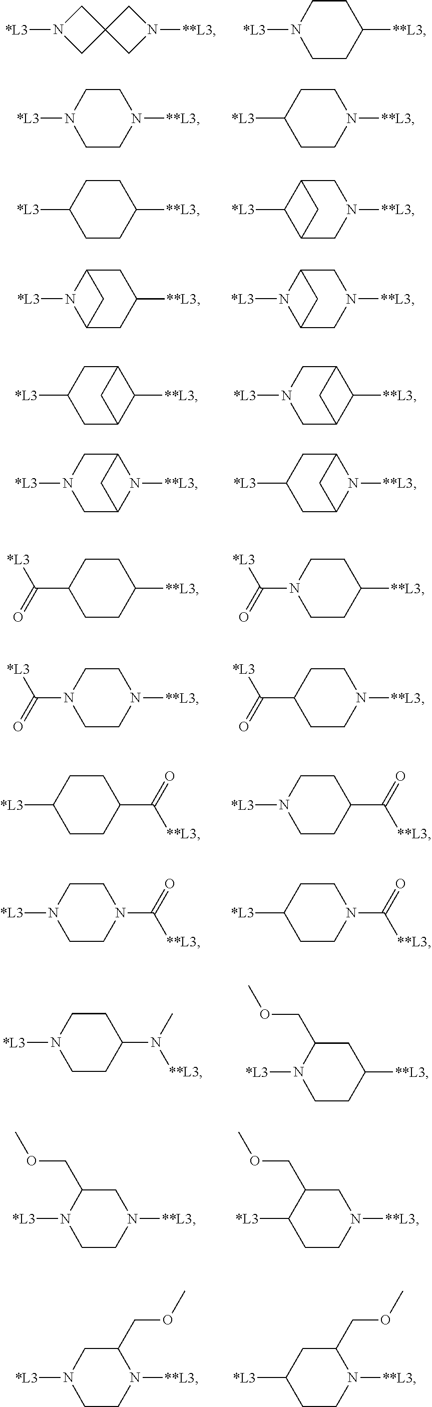

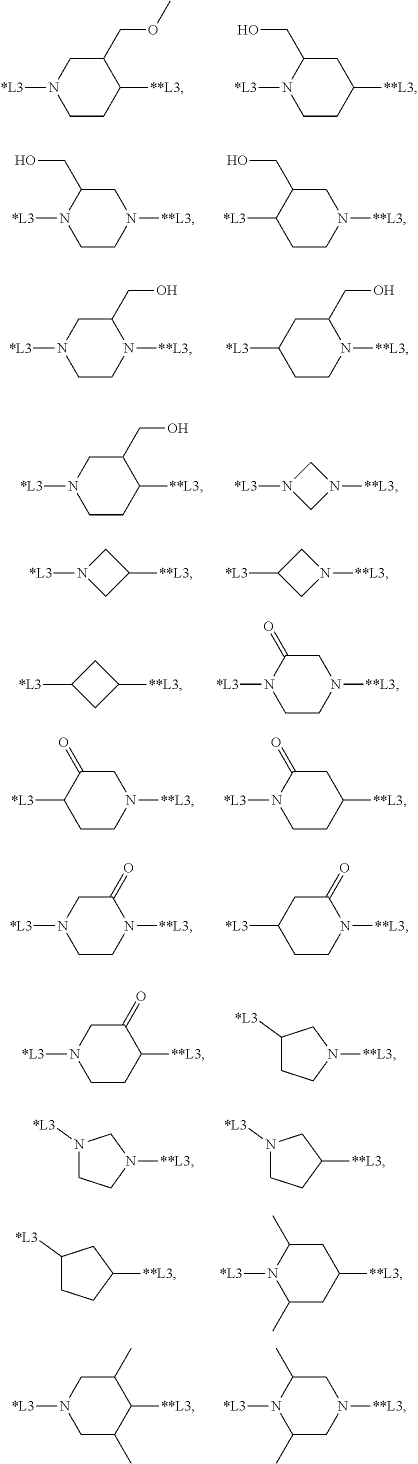

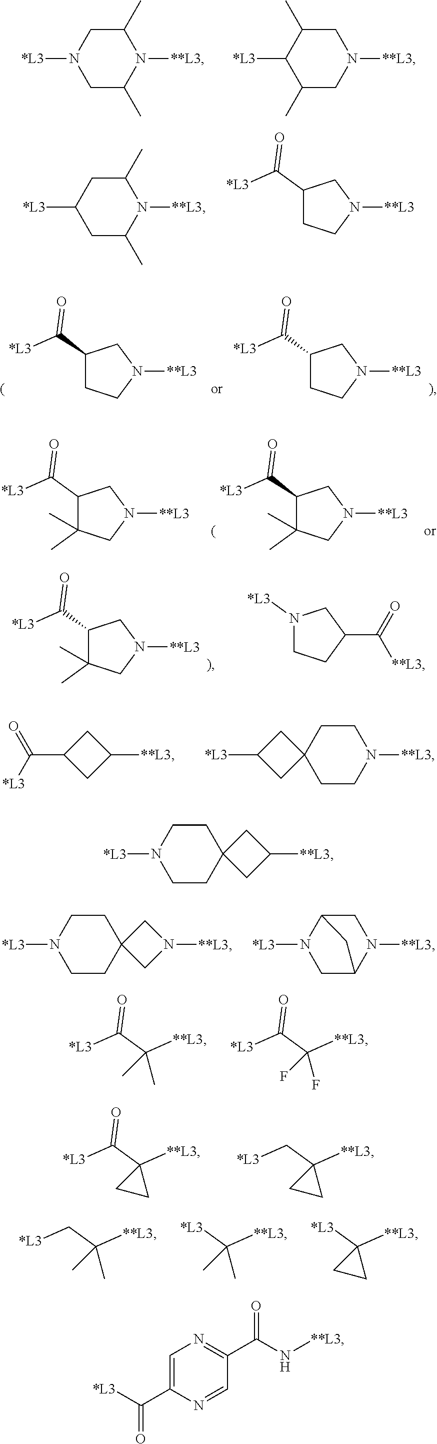

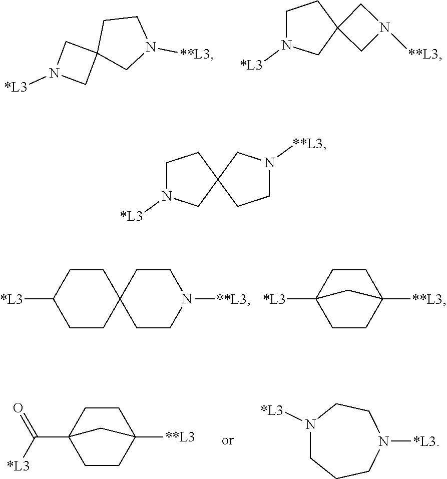

31. The compound of any one of claims 1-30, wherein L² is a single bond, L³ is a single bond, or L² and L3 are both single bond.

32. The compound of any one of claims 1-31, wherein 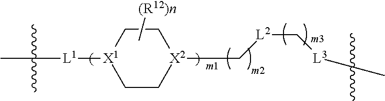
is selected from

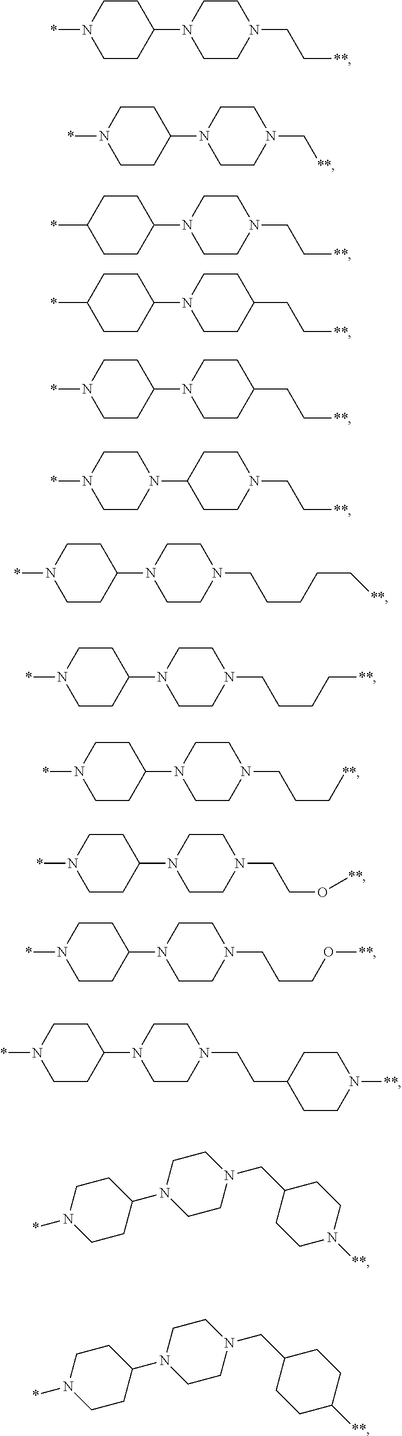

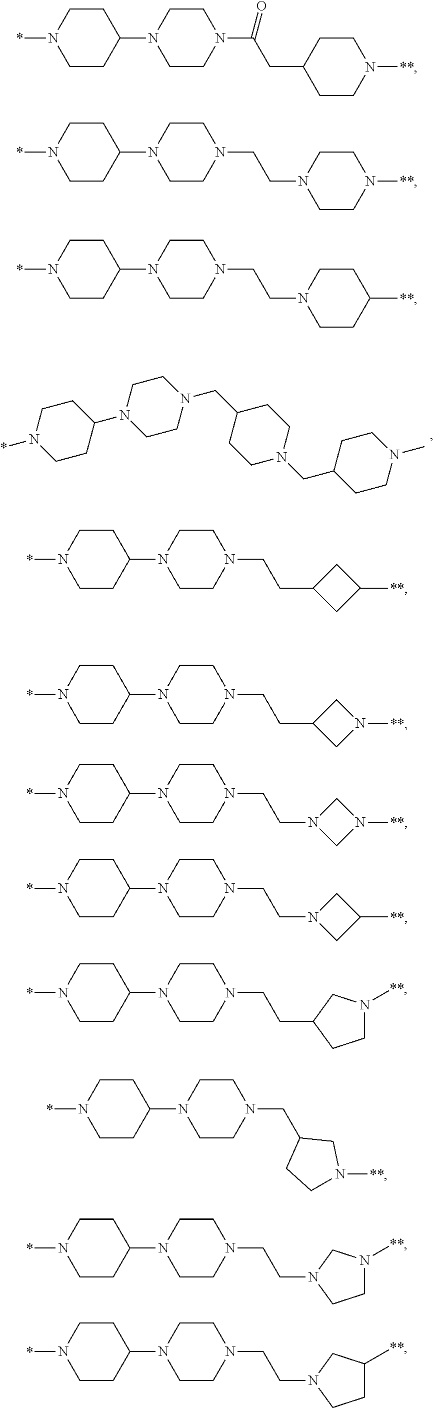

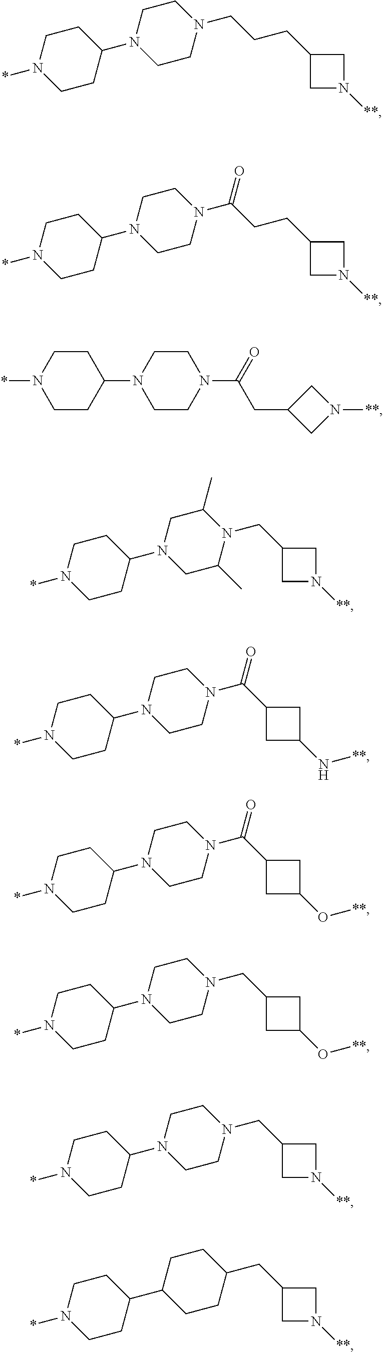

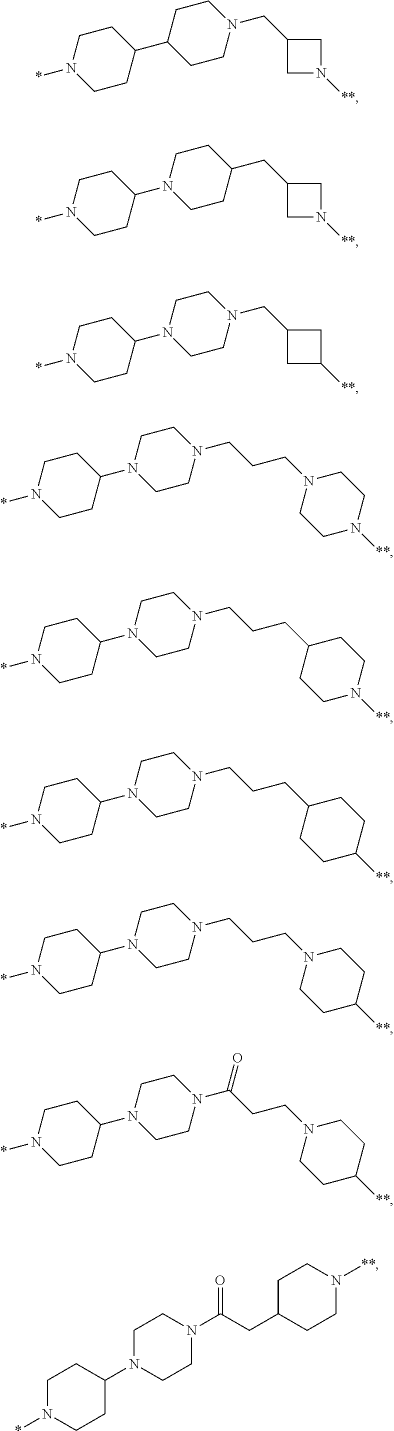

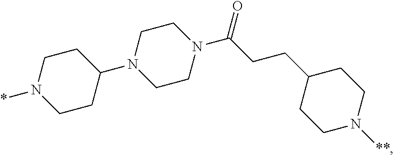

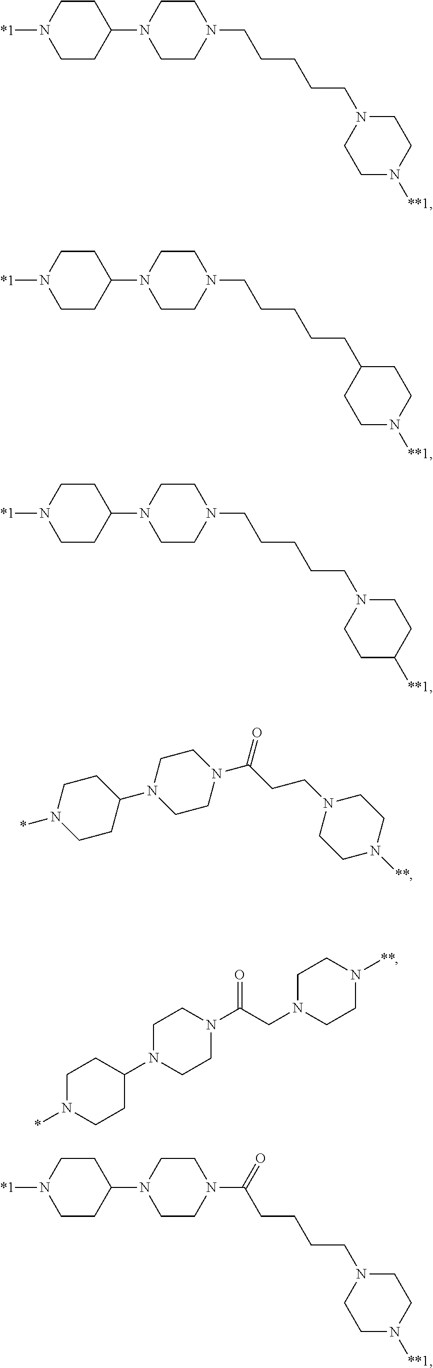

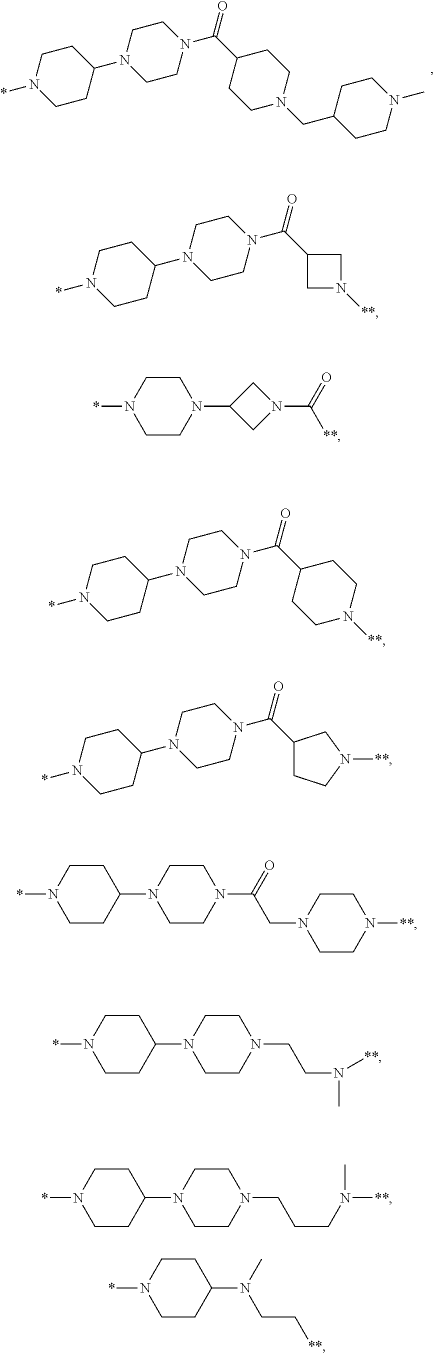

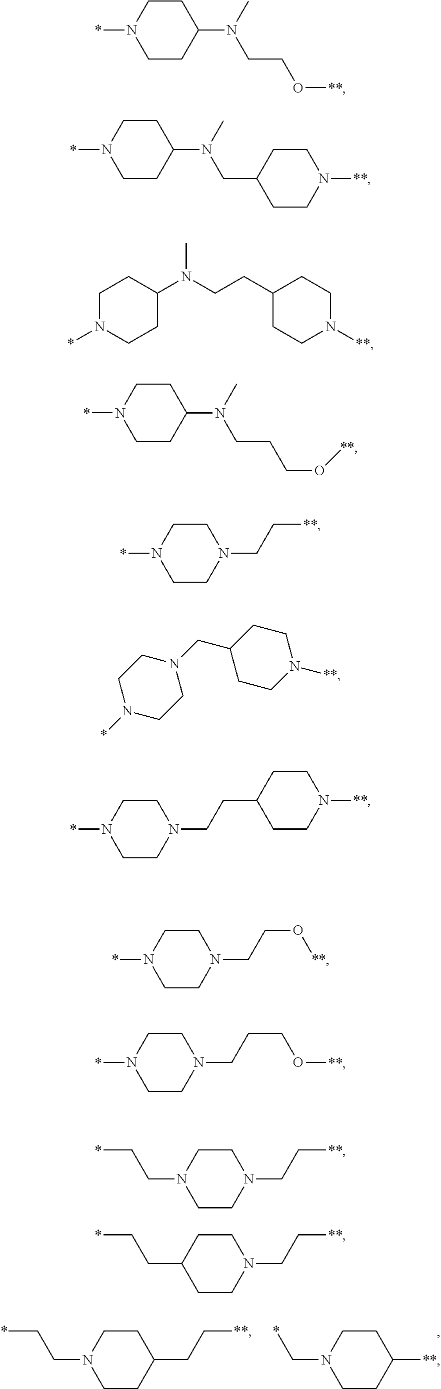

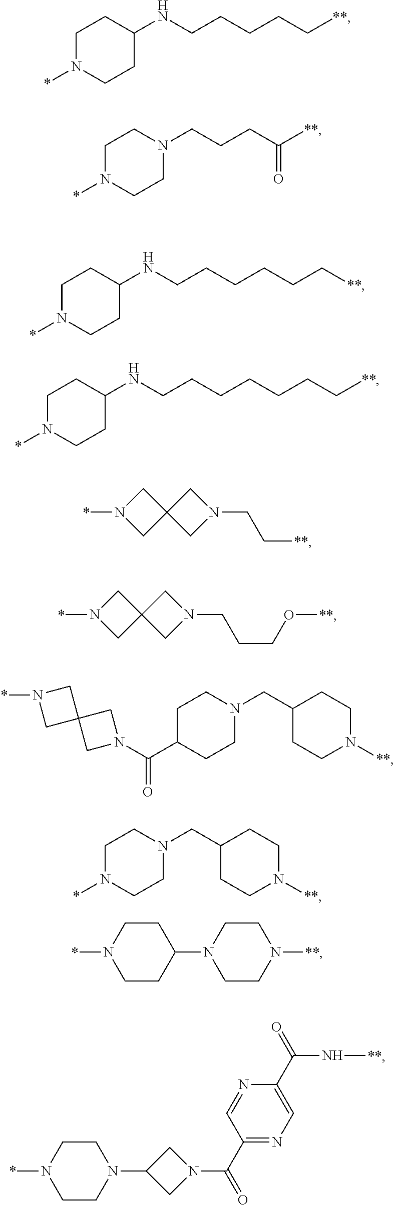

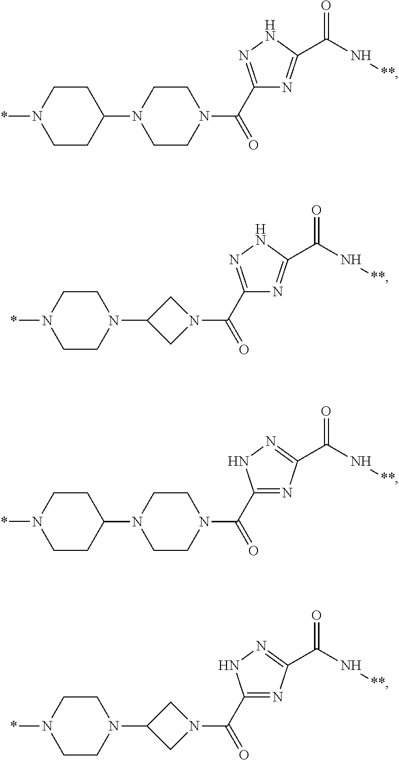

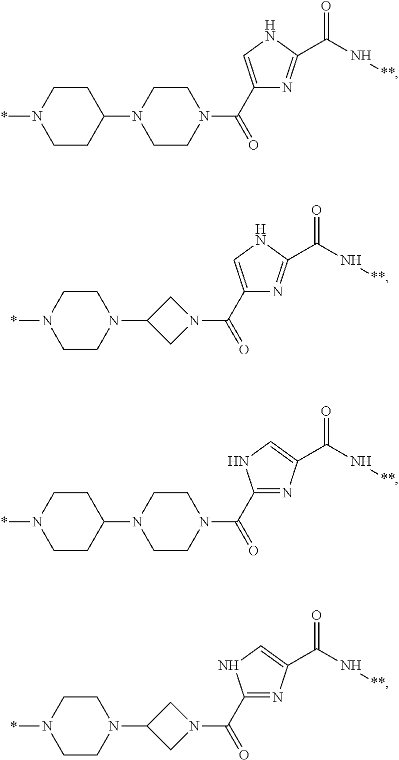

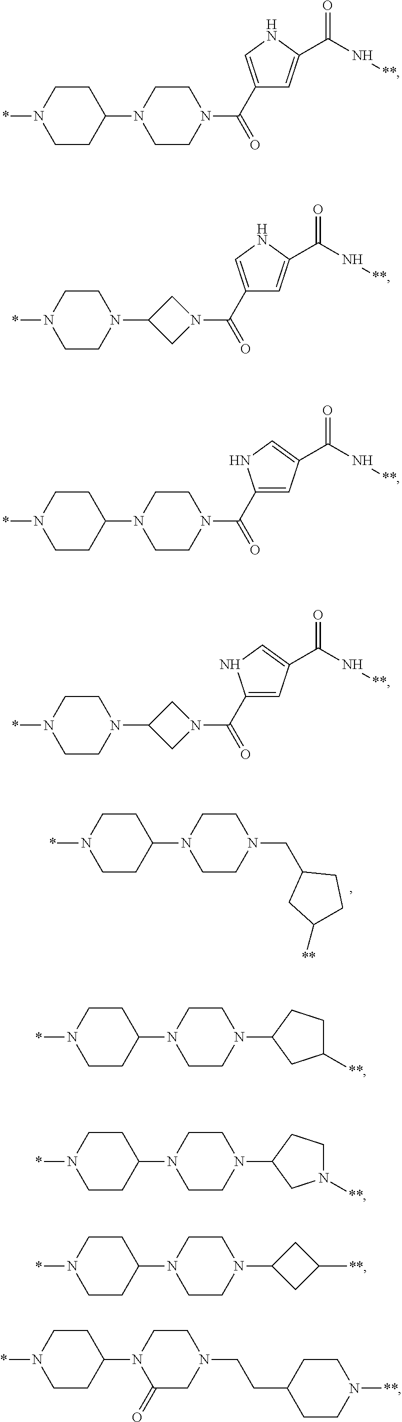

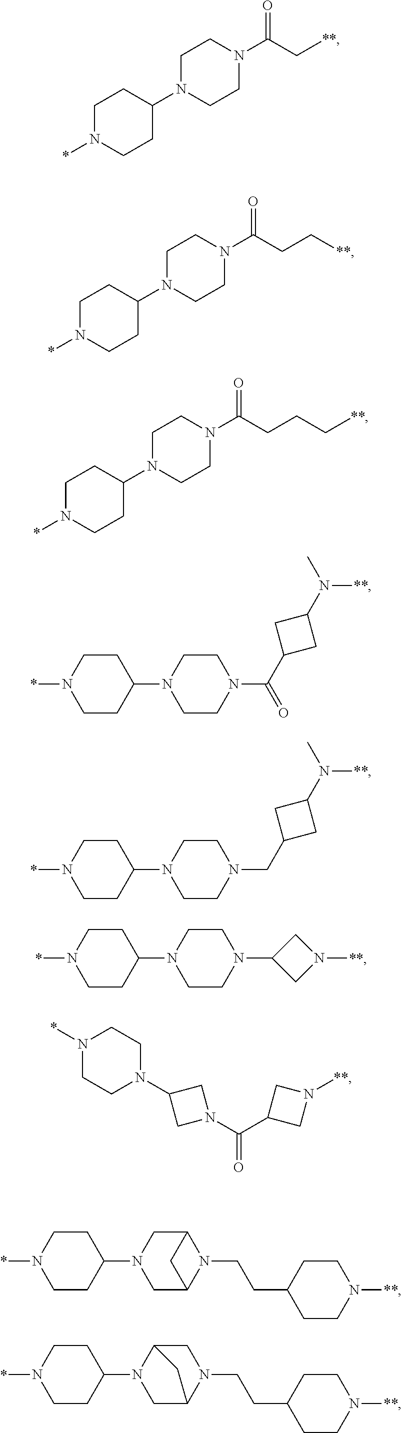

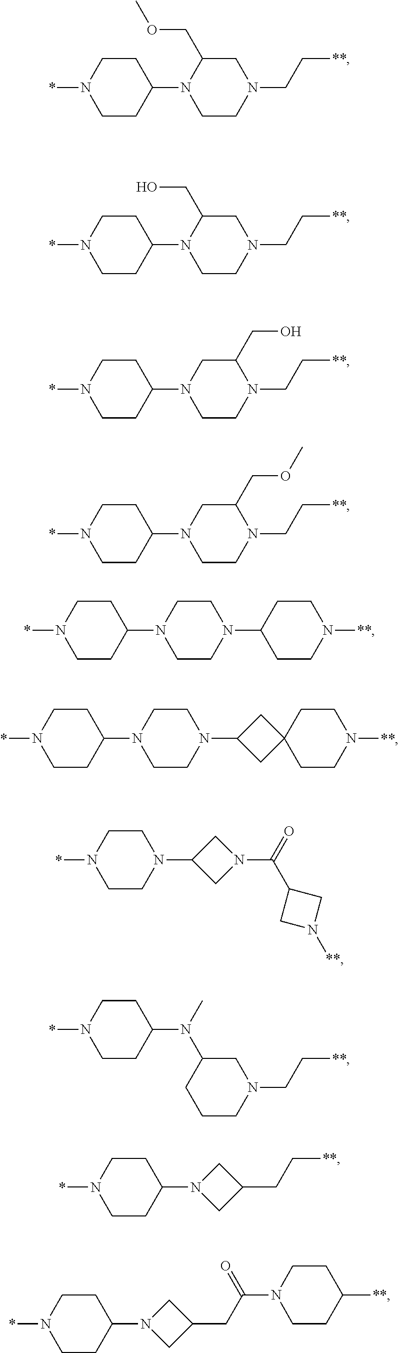

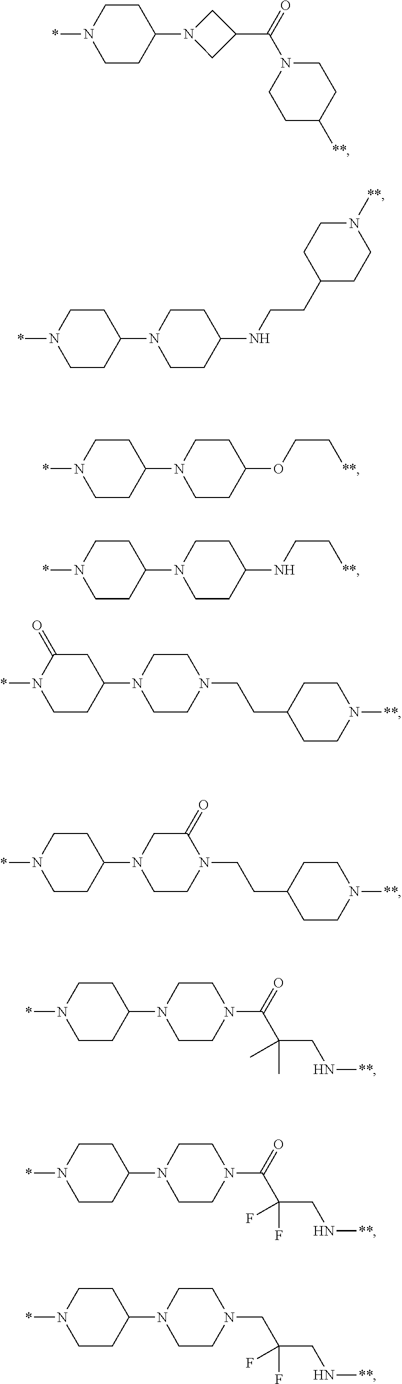

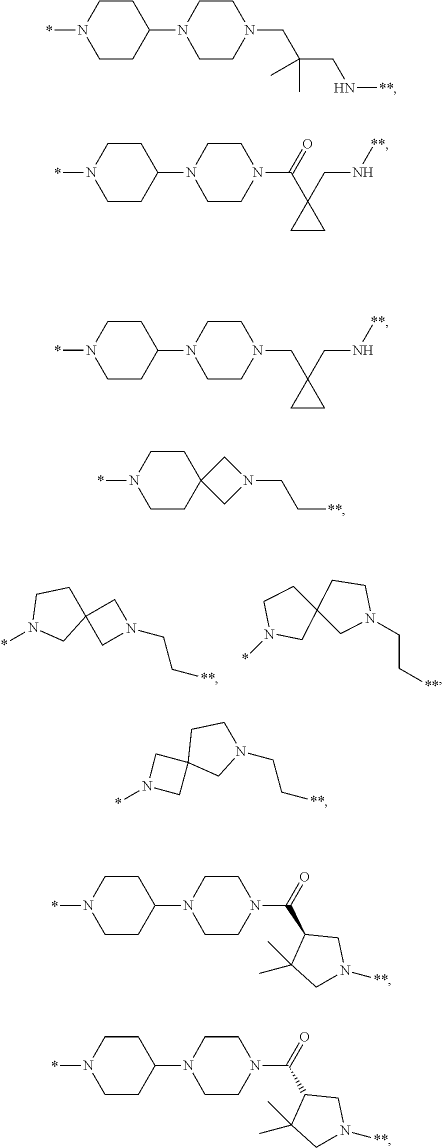

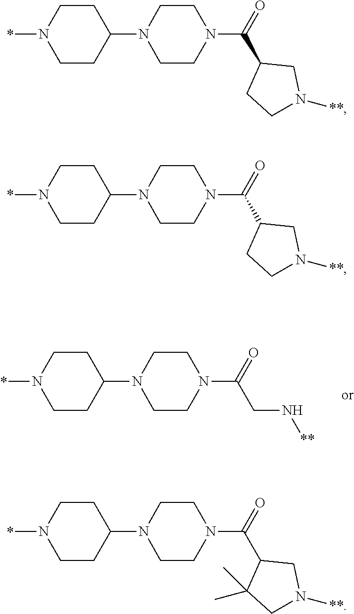

33. The compound of any one of claims 1-32, wherein R¹³, R¹⁴, R¹⁵, R¹⁶ and R¹⁷ are each independently selected from hydrogen, —F, —Cl, —Br, —I, CN, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl; said each methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent —F, —Cl, —Br, —I, methyl, ethyl, propyl, butyl, pentyl, hexyl, heptyl, octyl, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl.

34. The compound of any one of claims 1-33, wherein at each occurrence, R^{a} and R^{b} are each independently selected from hydrogen, —F, —Cl, —Br, —I, CN, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl; or
R^{a} and R^{b} together with the carbon atoms to which they are attached, form a 3, 4, 5, 6, 7 or 8-membered ring, said ring comprising 0-3 heteroatoms independently selected from nitrogen, oxygen or sulfur; said ring is optionally substituted with at least one substituent —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl.

35. The compound of any one of claims 1-34, wherein 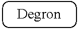
is selected from 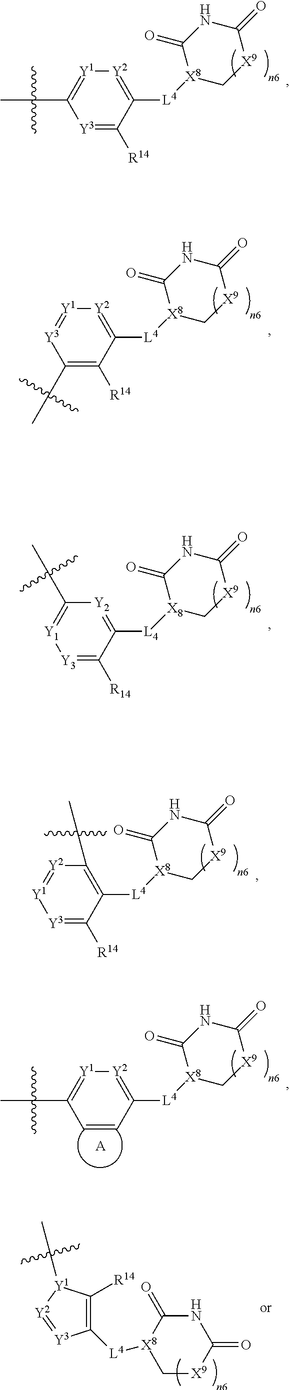 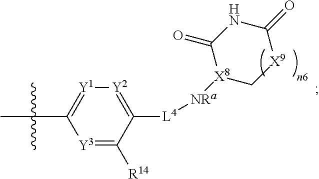
Ring A is selected from 5- to 7-membered cycloalkyl, 5- to 7-membered heterocyclyl, aryl, or heteroaryl;
R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen or —C₁-C₈alkyl; preferably R¹⁴ is independently selected from H, F, Cl, Br, I, CH₃, —OCH₃, —OCH₂F, —OCHF₂, —O CF₃, CH₂F, CN, CHF₂, or CF₃;
X⁸ is independently selected from CH, CD, C(CH₃), C(C₂H₅), C(C₃H₇), C(F) or N;
L⁴ is independently selected from a single bond, —O—, —NH—, —CH₂—, —CHF—, or —CF₂—;
Y¹, Y², and Y³ are each independently selected from CR^{a} or N;
X⁹ is CH₂;
R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen, hydroxy, —C₁-C₈alkyl, or —C₁-C₈alkoxy; and
n₆ is independently 0,1 or 2.

36. The compound of any one of claims 1-35, wherein 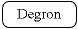
is selected from 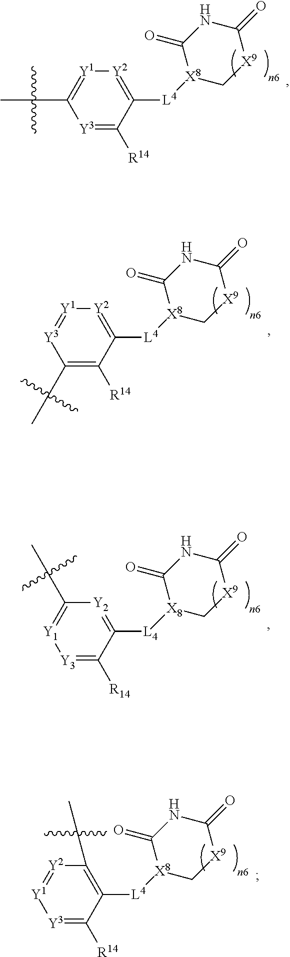
R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen; preferably R¹⁴ is independently selected from H, F, Cl, Br, I, CH₃, —OCH₃, —OCH₂F, —OCHF₂, —O CF₃, CH₂F, CN, CHF₂, or CF₃;
X⁸ is independently selected from CH, CD, C(CH₃), C(C₂H₅), C(C₃H₇), C(F) or N;
L⁴ is a single bond;
Y¹, Y², and Y³ are each independently selected from CR^{a} or N;
X⁹ is CH₂;
R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen; and
n₆ is 1.

37. The compound of any one of claims 1-36, wherein 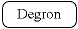
is selected from 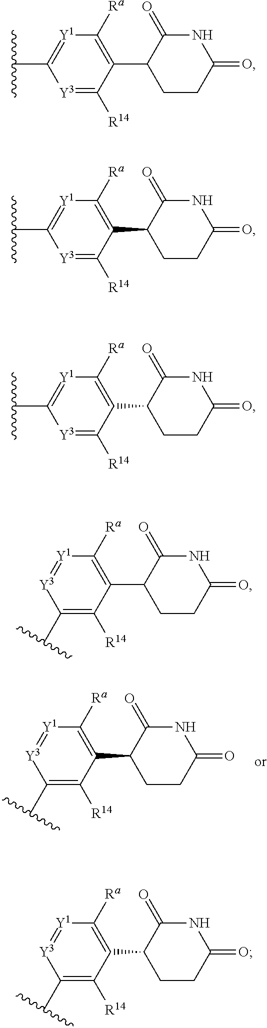
R¹⁴ is independently selected from hydrogen, halogen, —C₁-C₈alkyl, —C₁-C₈alkoxy, or CN; said each —C₁-C₈alkyl, or —C₁-C₈alkoxy is optionally substituted by one or more halogen; preferably R¹⁴ is independently selected from H, F, Cl, Br, I, CH₃, —OCH₃, —OCH₂F, —OCHF₂, —O CF₃, CH₂F, CN, CHF₂, or CF₃;
Y¹ and Y³ are each independently selected from CH or N;
R^{a} is each independently selected from hydrogen, halogen, —C₁-C₈alkyl, or —C₁-C₈alkoxy, each of said —C₁-C₈alkyl or —C₁-C₈alkoxy is optionally substituted with at least one or more halogen.

38. The compound of any one of claims 1-37, wherein 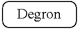
is selected from 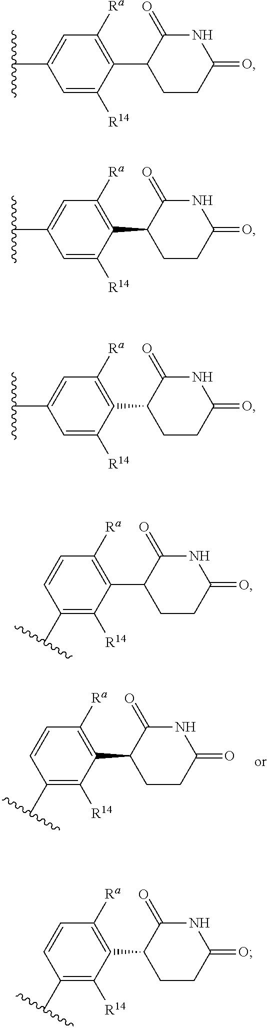
R¹⁴ is independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, or CN; said each methyl, ethyl, propyl, butyl, pentyl, hexyl, heptyl, octyl, methoxy, ethoxy, propoxy, butoxy, pentoxy, hexoxy, heptyloxy, octyloxy is optionally substituted by one or more F, Cl, Br, I; preferably R¹⁴ is independently selected from H, F, Cl, Br, I, CH₃, —OCH₃, —OCH₂F, —OCHF₂, —O CF₃, CH₂F, CN, CHF₂, or CF₃;
R^{a} is each independently selected from hydrogen, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, each of said methyl, ethyl, propyl, butyl, pentyl, hexyl, heptyl, octyl, methoxy, ethoxy, propoxy, butoxy, pentoxy, hexoxy, heptyloxy, octyloxy is optionally substituted with at least one or more F, Cl, Br, I.

39. The compound of any one of claims 1-38, wherein 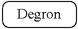
is selected from 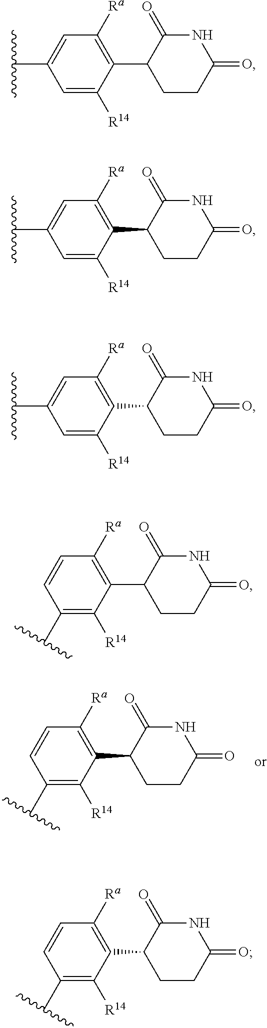
R¹⁴ is independently selected from F, Cl, Br, I, —CH₃, —OCH₃, —OCH₂F, —OCHF₂, —O CF₃, CH₂F, CN, CHF₂, or CF₃;
R^{a} is each independently selected from F, Cl, Br, I, —CH₃, —OCH₃, —OCH₂F, —OCHF₂, —O CF₃, CH₂F, CN, CHF₂, or CF₃.

40. The compound of any one of claims 1-39, wherein 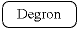
is 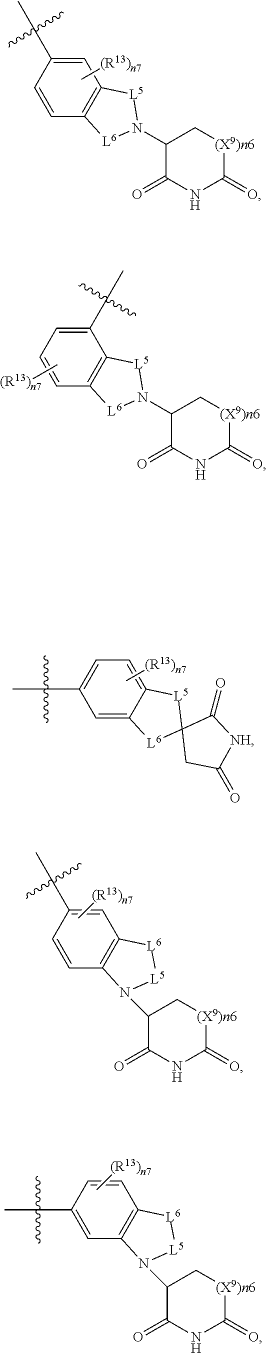 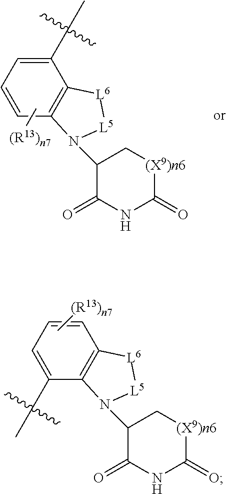
Wherein L⁵ and L⁶ are independently selected from a single bond, —O—, —NR^{a}—, —(CR^{a}R^{b})n₈-, —O(CR^{a}R^{b})n₈-, —NR^{a}(CR^{a}R^{b})n₈- or —C(O)—;
X⁹ is —CR^{a}R^{b}—;
R^{a} and R^{b} are each independently selected from hydrogen, hydroxy, F, Cl, Br, I, CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl, each of said methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl and 5- to 12-membered heteroaryl is optionally substituted with at least one substituent halogen, hydroxy, F, Cl, Br, I, CN, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, —C₆-C₁₂aryl or 5- to 12-membered heteroaryl; or
R^{a} and R^{b} together with the carbon atoms to which they are attached, form a 3-, 4-, 5-, 6-, 7-, 8-membered ring, said ring comprising 0-3 heteroatoms independently selected from nitrogen, oxygen or sulfur; said ring is optionally substituted with at least one substituent halogen, hydroxy, F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl;
each R¹³ is independently selected from hydrogen, halogen, CN, —C₁-C₈alkyl, or —C₁-C₈alkoxy;
n₆ is 0 or 1; and
n₇ is 0, 1 or 2.

41. The compound of any one of claims 1-40, wherein 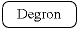
is 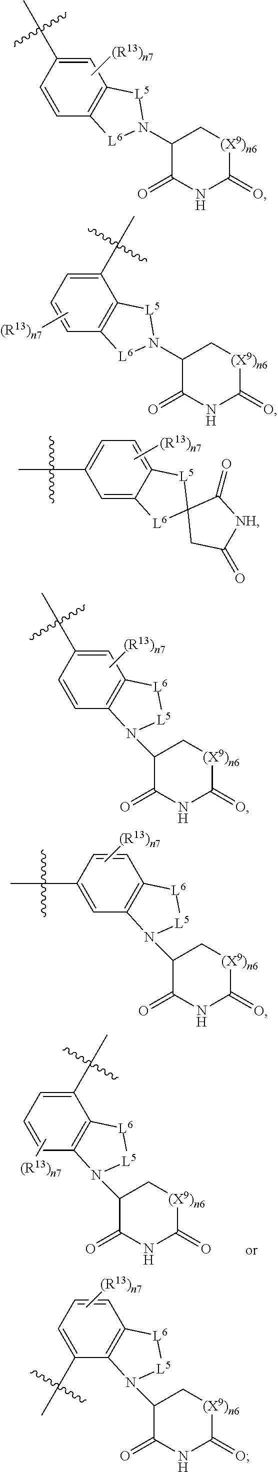
Wherein L⁵ and L⁶ is independently selected from a single bond, 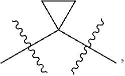 [SMILES: *C1(*)CC1]
—O—, —NH—, —NMe-, —N(CH₂CH₃)—, —N(ⁱPr)—, —CH₂—, —CHF—, —CF₂—, —C(CH₃)₂— or —C(O)— (preferably L⁵ is —C(O)— or —CH₂—, and L⁶ is 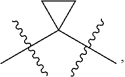 [SMILES: *C1(*)CC1]
—O—, —NH—, —NMe-, —N(CH₂CH₃)—, —N(ⁱPr)—, 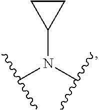 [SMILES: *N(*)C1CC1]
—CH₂—, —CHF—, —CF₂—, —C(CH₃)₂— or —C(O)—);
X⁹ is CH₂;
each R¹³ is independently selected from hydrogen, halogen, CN, —C₁-C₈alkyl, or —C₁-C₈alkoxy;
n₆ is 0 or 1; and
n₇ is 0, 1 or 2.

42. compound of any one of claims 1-41, wherein 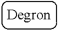
is 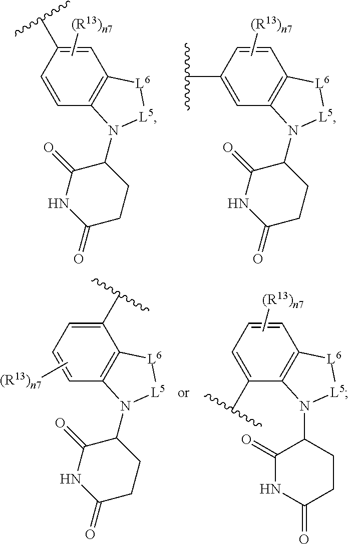
Wherein L⁵ and L⁶ are each independently selected from 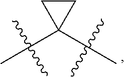 [SMILES: *C1(*)CC1]
—O—, —NH—, —NMe-, —N(CH₂CH₃)—, —N(ⁱPr)—, —CH₂—, —CHF—, —CF₂—, —C(CH₃)₂— or —C(O)—;
each R¹³ is independently selected from hydrogen, F, Cl, Br, I, CN, -Me, -Et, —C₃H₇, —C₄H₉, —OMe, —OCH₂F, —OCHF₂, —O CF₃, —OEt, —OC₃H₇ or —OC₄H₉;
n₇ is 0, 1 or 2.

43. The compound of any one of claims 1-42, wherein 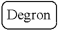
is 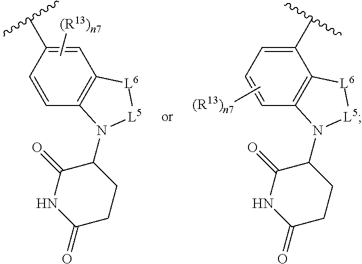
Wherein L⁶ is selected from 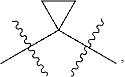 [SMILES: *C1(*)CC1]
—O—, —NMe-, —N(CH₂CH₃)—, —N(ⁱPr)—, —CH₂—, —CHF—, —CF₂— or —C(CH₃)₂—;
Wherein L⁵ is —C(O)—;
each R¹³ is independently selected from hydrogen, F, Cl, Br, I, CN, —C₁-C₈alkyl, or —C₁-C₈alkoxy;
n₇ is 0, 1 or 2.

44. The compound of any one of claims 1-43, wherein 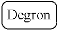
is 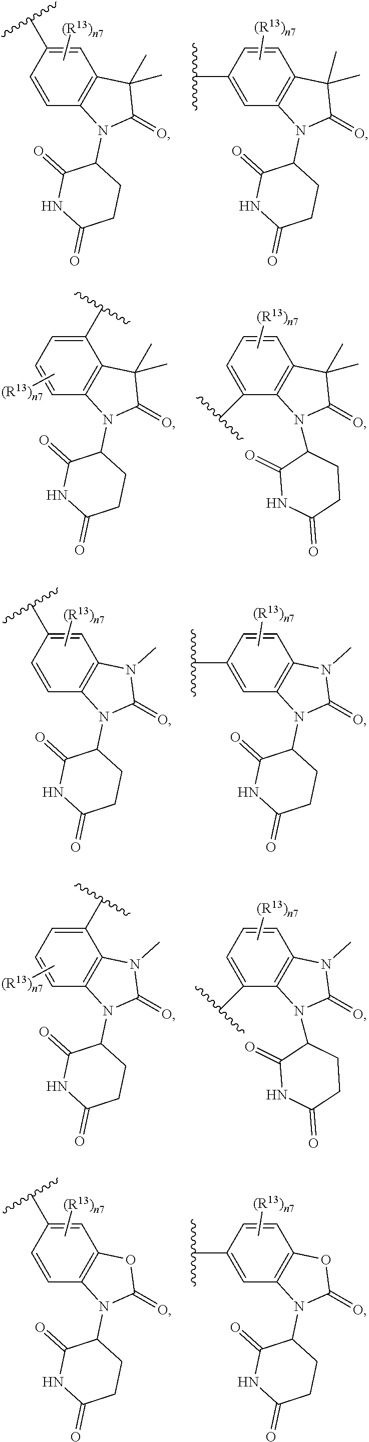 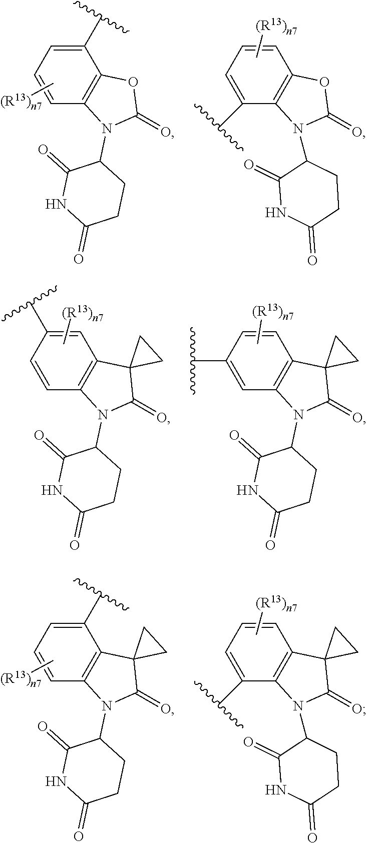
each R¹³ is independently selected from hydrogen, F, Cl, Br, I, CN, -Me, -Et, —C₃H₇, —C₄H₉, —OMe, —OCH₂F, —OCHF₂, —O CF₃, —OEt, —OC₃H₇ or —OC₄H₉;
n₇ is 0, 1 or 2.

45. The compound of any one of claims 1-44, wherein 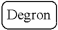
is 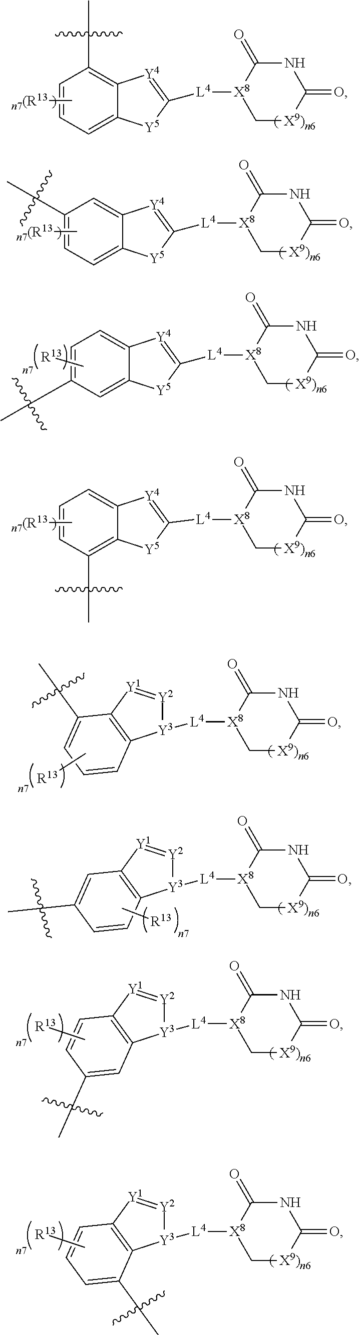 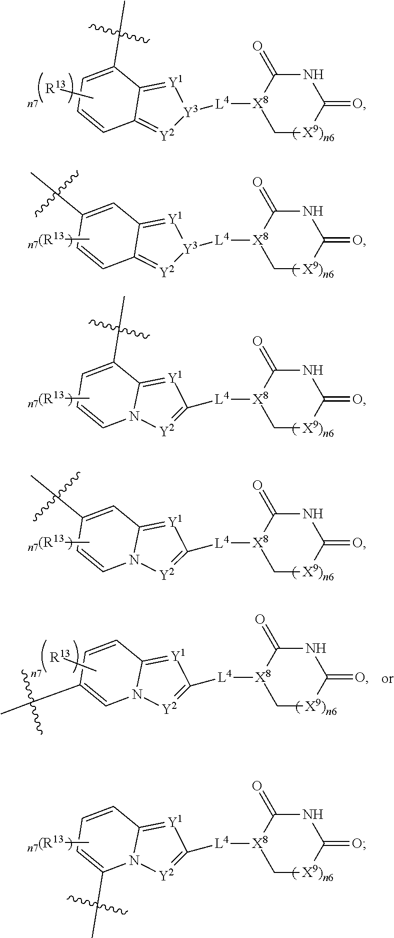
Wherein L⁴ is independently selected from a single bond, 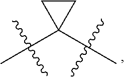 [SMILES: *C1(*)CC1]
—O—, —NH—, —CH₂—, —CHF—, or —CF₂—;
X⁸ is independently selected from CH, C(CH₃), C(C₂H₅), C(C₃H₇), C(F) or N;
X⁹ is CH₂;
each R¹³ is independently selected from hydrogen, halogen, CN, —C₁-C₈alkyl, or —C₁-C₈alkoxy;
Y¹, Y², Y³ and Y⁴ are each independently selected from CH, C(CH₃), C(F), or N;
Y⁵ is selected from NH, N(CH₃), O or S;
n₆ is 0 or 1; and
n₇ is 0, 1 or 2.

46. The compound of any one of claims 1-45, wherein 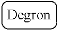
is selected from

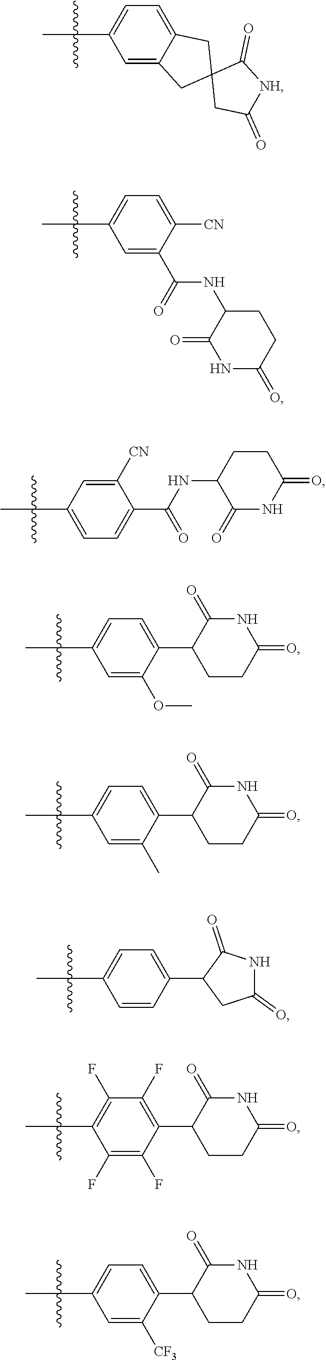

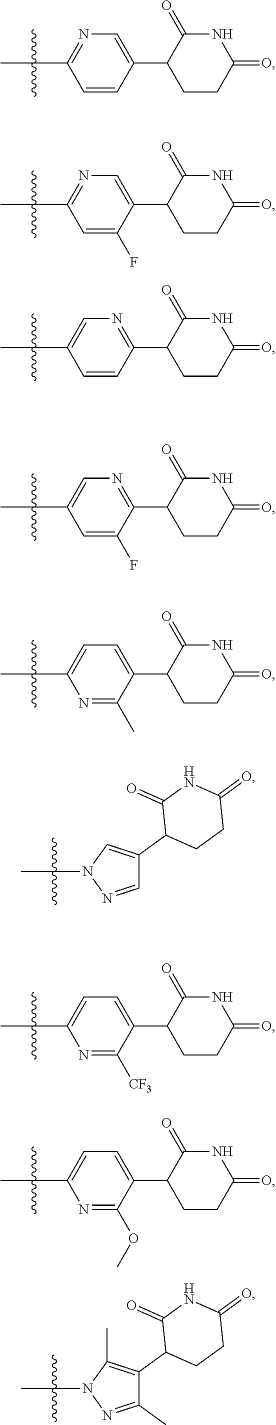

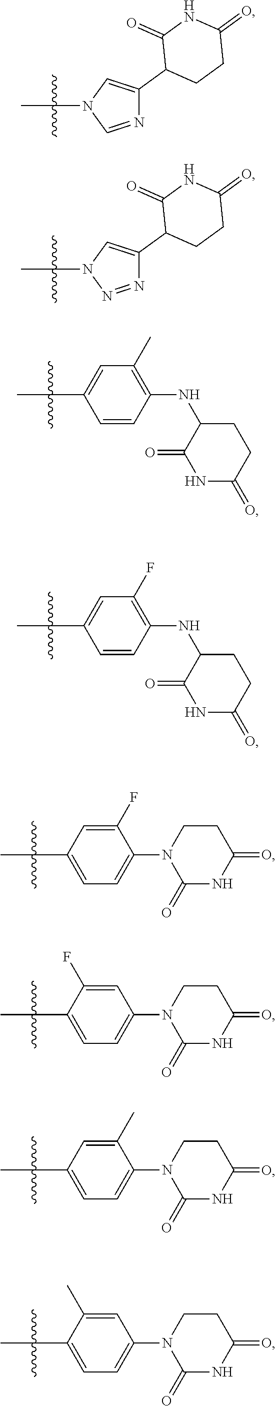

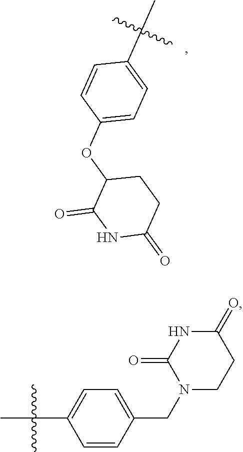 [SMILES: *c1ccc(OC2CCC(=O)NC2=O)cc1 ; *c1ccc(CN2CCC(=O)NC2=O)cc1]

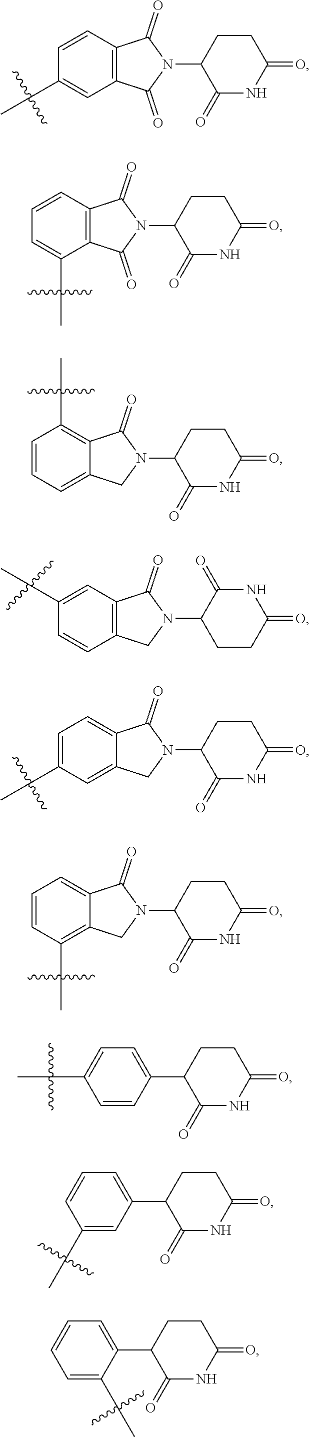

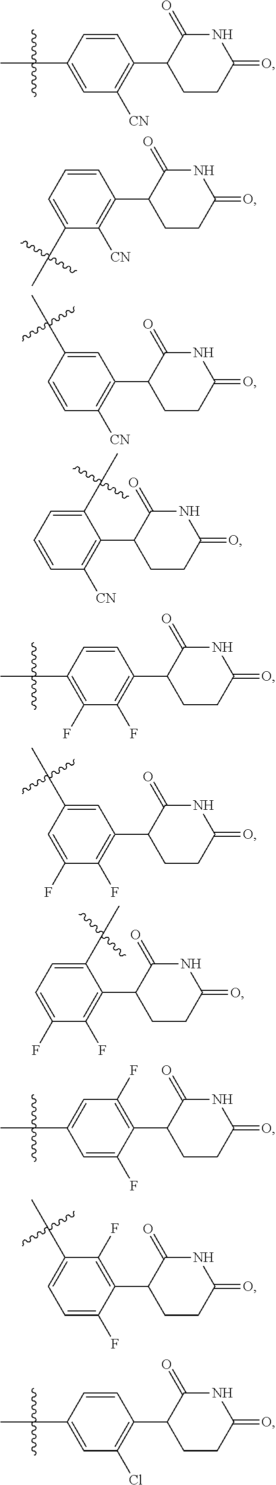

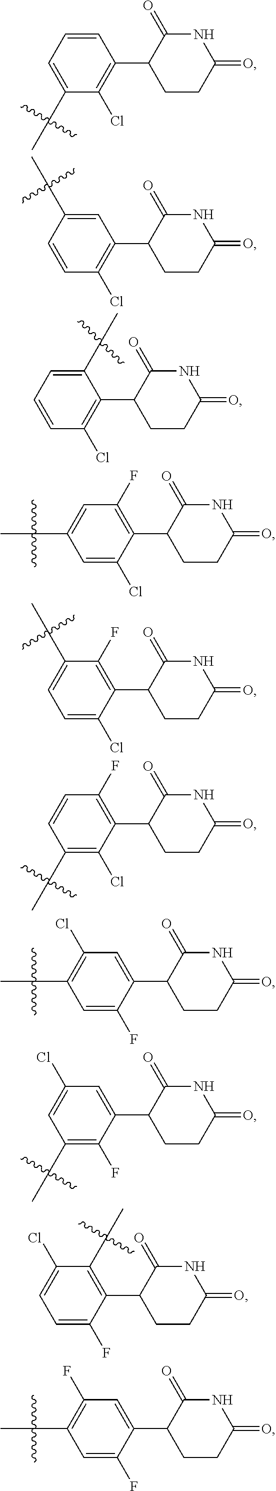

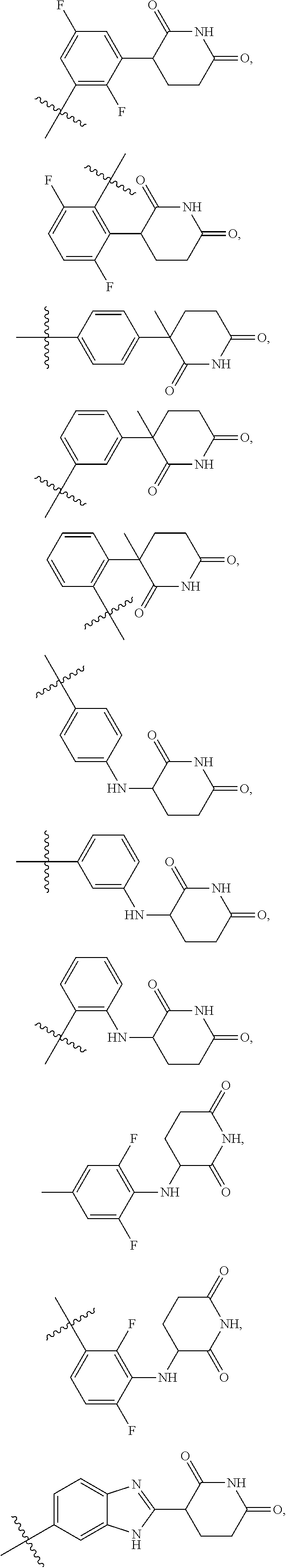

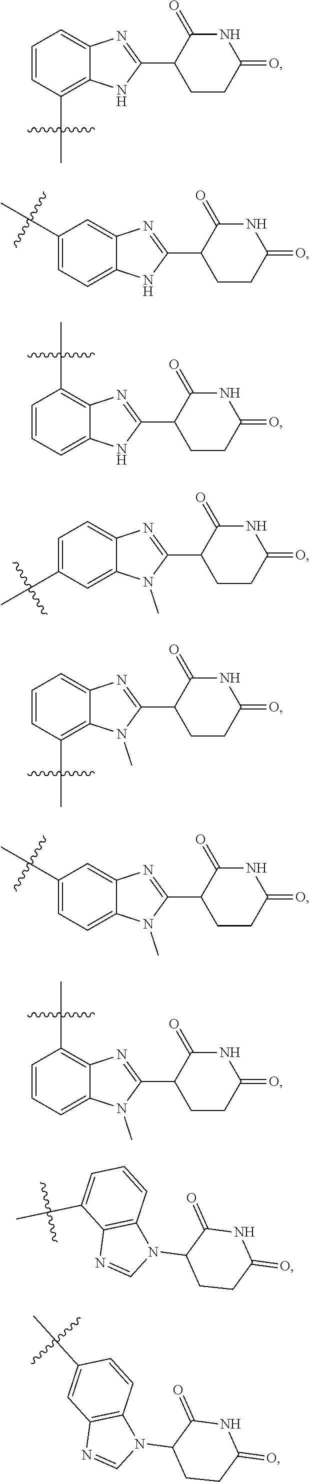

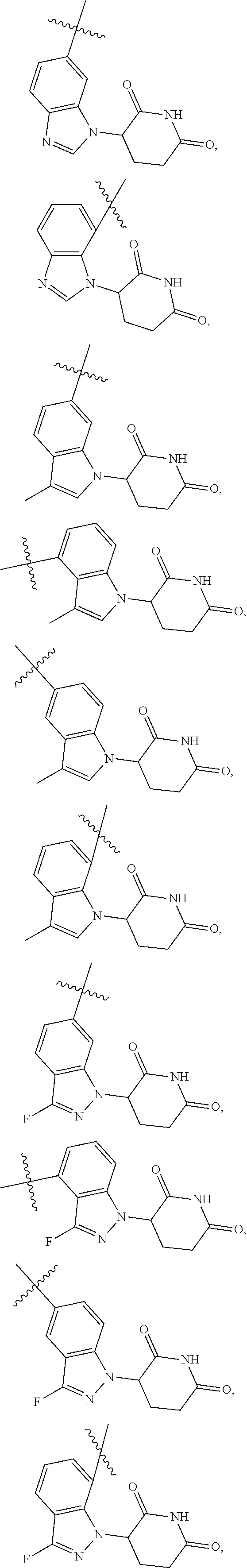

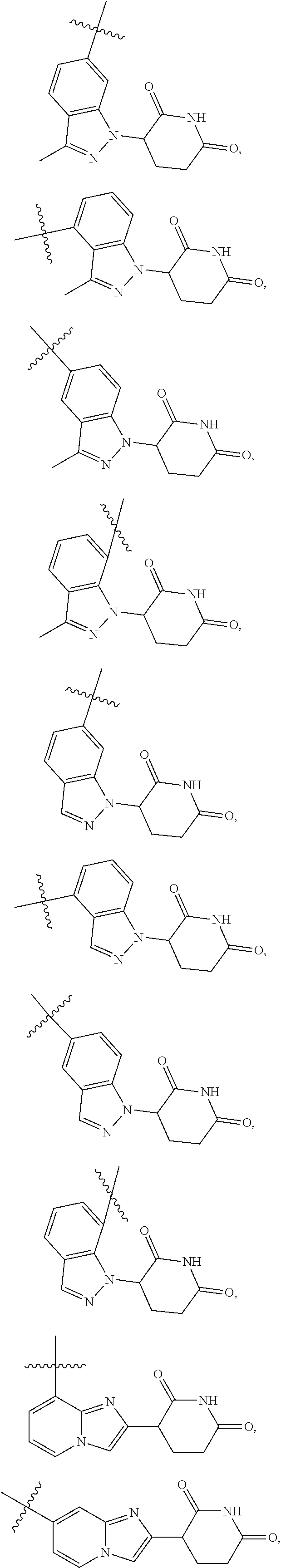

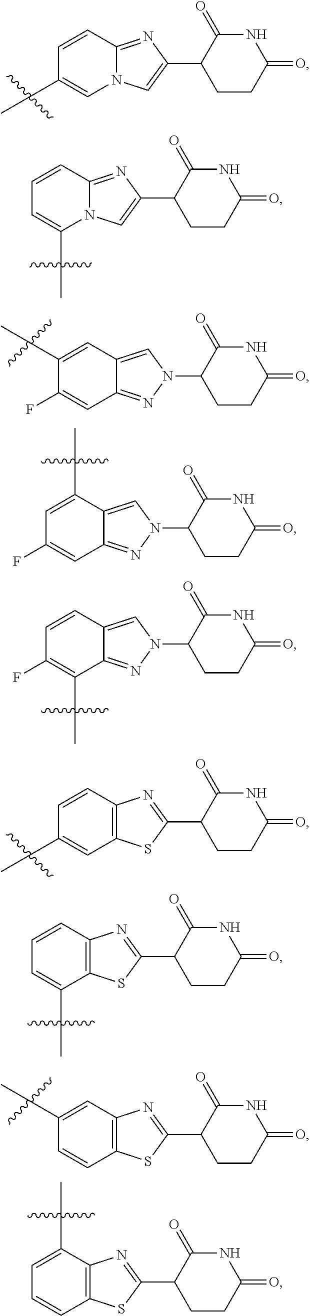

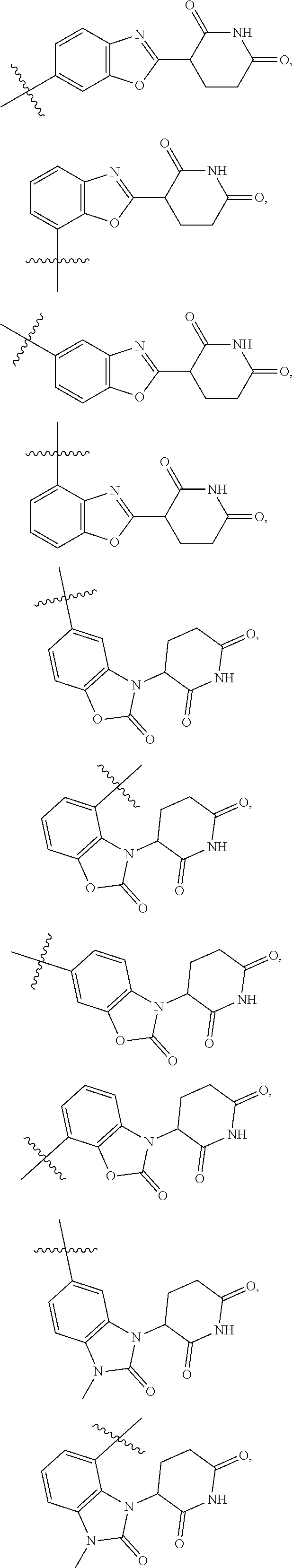

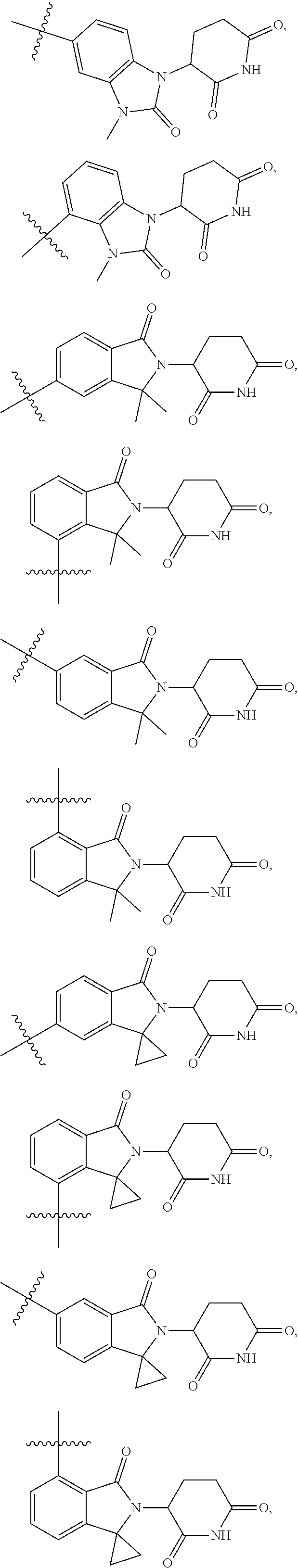

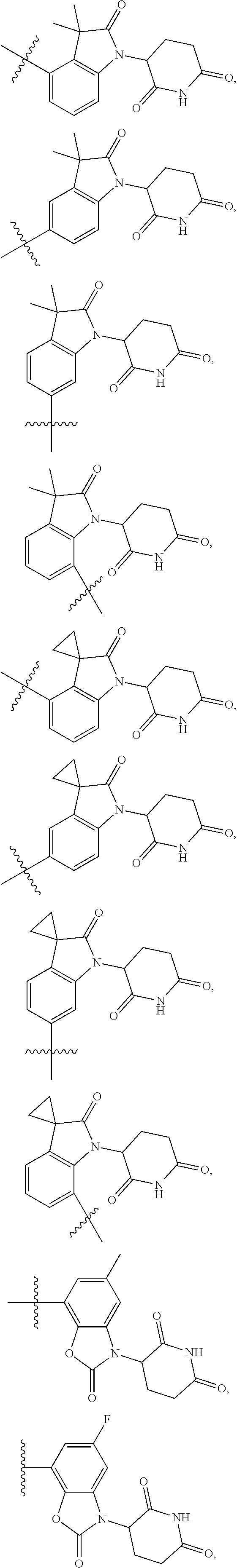

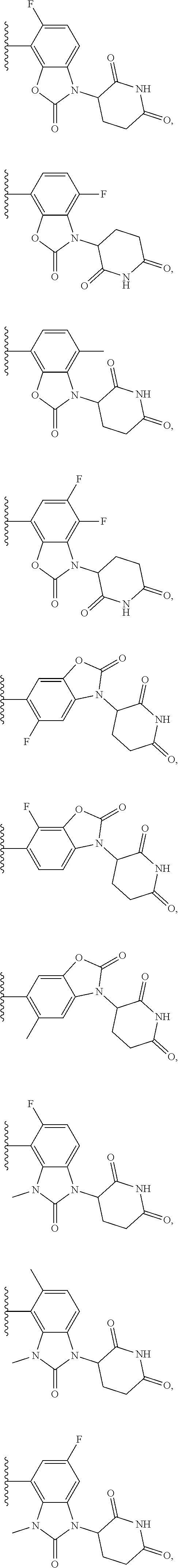

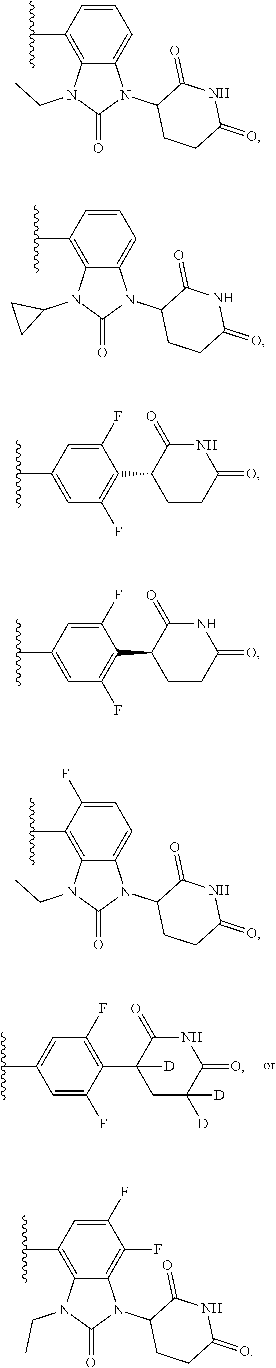

47. The compound of any one of claims 1-46, wherein Z¹, Z², Z³ and Z⁴ are each independently —CR^{Z};
R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one R^{Zc};
R^{Za} and R^{Zb} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Zc} and R^{Zd} are each independently —F, —Cl, —Br, —I, hydroxy, methyl, ethyl, propyl, butyl, pentyl, hexyl, heptyl, octyl, —C₁-C₈alkoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

48. The compound of any one of claims 1-47, wherein R^{Z}, at each occurrence, is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CH(OH)CH₃

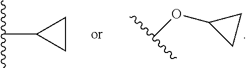

49. The compound of any one of claims 1-48 selected from the compound of Example 1, Example 2, Example 3, Example 4, Example 5, Example 6, Example 7, Example 8, Example 9, Example 10, Example 11, Example 12, Example 13, Example 14, Example 15, Example 16, Example 17, Example 18, Example 19, Example 20, Example 21, Example 22, Example 23, Example 24, Example 25, Example 26, Example 27, Example 28, Example 29, Example 30, Example 31, Example 32, Example 33, Example 34, Example 35, Example 36, Example 37, Example 38, Example 39, Example 40, Example 41, Example 42, Example 43, Example 44, Example 45, Example 46, Example 47, Example 48, Example 49, Example 50, Example 51, Example 52, Example 53, Example 54, Example 55, Example 56, Example 57, Example 58, Example 59, Example 60, Example 61, Example 62, Example 63, Example 64, Example 65, Example 66, Example 67, Example 68, Example 69, Example 70, Example 71, Example 72, Example 73, Example 74, Example 75, Example 76, Example 77, Example 78, Example 79, Example 80, Example 81, Example 82, Example 83, Example 84, Example 85, Example 86, Example 87, Example 88, Example 89, Example 90, Example 91, Example 92, Example 93, Example 94, Example 95, Example 96, Example 97, Example 98, Example 99, Example 100, Example 101, Example 102, Example 103, Example 104, Example 105, Example 106, Example 107, Example 108, Example 109, Example 110, Example 111, Example 112, Example 113, Example 114, Example 115, Example 116, Example 117, Example 118, Example 119, Example 120, Example 121, Example 122, Example 123, Example 124, Example 125, Example 126, Example 127, Example 128, Example 129, Example 130, Example 131, Example 132, Example 133, Example 134, Example 135, Example 136, Example 137, Example 138, Example 139, Example 140, Example 141, Example 142, Example 143, Example 144, Example 145, Example 146, Example 147, Example 148, Example 149, Example 150, Example 151, Example 152, Example 153, Example 154, Example 155, Example 156, Example 157, Example 158, Example 159, Example 160, Example 161, Example 162, Example 163, Example 164, Example 165, Example 166, Example 167, Example 168, Example 169, Example 170, Example 171, Example 172, Example 173, Example 174, Example 175, Example 176, Example 177, Example 178, Example 179, Example 180, Example 181, Example 182, Example 183, Example 184, Example 185, Example 186, Example 187, Example 188, Example 189, Example 190, Example 191, Example 192, Example 193, Example 194, Example 195, Example 196, Example 197, Example 198, Example 199, Example 200, Example 201, Example 202, Example 203, Example 204, Example 205, Example 206, Example 207, Example 208, Example 209, Example 210, Example 211, Example 212, Example 213, Example 214, Example 215, Example 216, Example 217, Example 218, Example 219, Example 220, Example 221, Example 222, Example 223, Example 224, Example 225, Example 226, Example 227, Example 228, Example 229, Example 230, Example 231, Example 232, Example 233, Example 234, Example 235, Example 236, Example 237, Example 238, Example 239, Example 240, Example 241, Example 242, Example 243, Example 244, Example 245, Example 246, Example 247, Example 248, Example 249, Example 250, Example 251, Example 252, Example 253, Example 254, Example 255, Example 256, Example 257, Example 258, Example 259, Example 260, Example 261, Example 262, Example 263, Example 264, Example 265, Example 266, Example 267, Example 268, Example 269, Example 270, Example 271, Example 272, Example 273, Example 274, Example 275, Example 276, Example 277, Example 278, Example 279, Example 280, Example 281, Example 282, Example 283, Example 284, Example 285, Example 286, Example 287, Example 288, Example 289, Example 290, Example 291, Example 292, Example 293, Example 294, Example 295, Example 296, Example 297, Example 298, Example 299, Example 300, Example 301, Example 302, Example 303, Example 304, Example 305, Example 306, Example 307, Example 308, Example 309, Example 310, Example 311, Example 312, Example 313, Example 314, Example 315, Example 316, Example 317, Example 318, Example 319, Example 320, Example 321, Example 322, Example 323, Example 324, Example 325, Example 326, Example 327, Example 328, Example 329, Example 330, Example 331, Example 332, Example 333, Example 334, Example 335, Example 336, Example 339, Example 340, Example 341, Example 342, Example 343, Example 344, Example 345, Example 346, Example 347, Example 348, Example 349, Example 350, Example 351, Example 352, Example 353, Example 354, Example 355, Example 356, Example 357, Example 358, Example 359, Example 360, Example 361, Example 362, Example 363, Example 364, Example 365, Example 366, Example 367, Example 368, Example 369, Example 370, Example 371, Example 372, Example 373, Example 374, Example 375, Example 376, Example 377, Example 378, Example 379, Example 380, Example 381, Example 382, Example 383, Example 384, Example 385, Example 386, Example 387, Example 388, Example 389, Example 390, Example 391, Example 392, Example 393, Example 394, Example 395, Example 396, Example 397, Example 398, Example 399, Example 400, Example 401, Example 402, Example 403, Example 404, Example 405, Example 406, Example 407, Example 408, Example 409, Example 410, Example 411, Example 412, Example 413, Example 414, Example 415, Example 416, Example 417, Example 418, Example 419, Example 420, Example 421, Example 422, Example 423, Example 424, Example 425, Example 426, Example 427, Example 428, Example 429, Example 430, Example 431, Example 432, Example 433, Example 434, Example 435, Example 436, Example 437, Example 438, Example 439, Example 440, Example 441, Example 442, Example 443, Example 444, Example 445, Example 446, Example 447, Example 448, Example 449, Example 450, Example 451, Example 452, Example 453, Example 454, Example 455, Example 456, Example 457, Example 458, Example 459, Example 460, Example 461, Example 462, Example 463, Example 465, Example 466, Example 467, Example 468, Example 469, Example 470, Example 471, Example 472, Example 473, Example 474, Example 476, Example 477, Example 478, Example 479, Example 480, Example 481, Example 482, Example 483, Example 484, Example 485, Example 486, Example 487, Example 488, Example 489, Example 490, Example 491, Example 492, Example 493, Example 496, Example 497, Example 498, Example 499, Example 500, Example 501, Example 502, Example 503, Example 504, Example 505, Example 506, Example 507, Example 508, Example 509, Example 510, Example 511, Example 512, Example 513, Example 514, Example 515, Example 516, Example 517, Example 518, Example 519, Example 520, Example 521, Example 522, Example 523, Example 525, Example 526, Example 527, Example 528, Example 529, Example 530, Example 531, Example 532, Example 533, Example 534, Example 535, Example 536, Example 537, Example 538, Example 539, Example 541, Example 542, Example 543, Example 544, Example 547, Example 548, Example 549, Example 550, Example 551, Example 552, Example 553, Example 554, Example 555, Example 556, Example 557, Example 558, Example 559, Example 560, Example 561, Example 562, Example 563, Example 564, Example 565, Example 566, Example 567, Example 568, Example 569, Example 570, Example 571, Example 572, Example 573, Example 574, Example 575, Example 576, Example 577, Example 578, Example 581, Example 582, Example 583, Example 584, Example 585, Example 586, Example 587, Example 588, Example 589, Example 590, Example 591, Example 592, Example 593, Example 594, Example 595, Example 596, Example 597, Example 598, Example 599, Example 600, Example 601, Example 602, Example 603, Example 604, Example 605, Example 607, Example 608, Example 609, Example 610, Example 611, Example 612, Example 613, Example 614, Example 615, Example 616, Example 617, Example 618, Example 619, Example 620, Example 621, Example 622, Example 623, Example 624, Example 625, Example 626, Example 627, Example 628, Example 629, Example 630, Example 631, Example 632, Example 633, Example 634, Example 635, Example 636, Example 637, Example 639, Example 640, Example 641, Example 642, Example 643, Example 644, Example 645, Example 646, Example 647, Example 648, Example 649, Example 650, Example 651, Example 652, Example 653, Example 654, Example 655, Example 656, Example 657, Example 658, Example 659, Example 660, Example 661, Example 662, Example 663, Example 665, Example 666, Example 667, Example 668, Example 669, Example 670, Example 671, Example 672, Example 673, Example 674, Example 675, Example 676, Example 677, Example 678, Example 679, Example 680, Example 681, Example 682, Example 683, Example 684, Example 685, Example 686, Example 687, Example 689, Example 690, Example 691, Example 692, Example 693, Example 694, Example 695, Example 696, Example 697, Example 698, Example 699, Example 700, Example 701, Example 703, Example 704, Example 705, Example 706, Example 707, Example 708, Example 709, Example 710, Example 711, Example 712, Example 713, Example 714, Example 715, Example 716, Example 717, Example 718, Example 719, Example 720, Example 721, Example 723, Example 724, Example 726, Example 727, Example 728, Example 729, Example 730, Example 731, Example 732, Example 733, Example 734, Example 735, Example 736, Example 737, Example 738, Example 739, Example 740, Example 741, Example 742, Example 743, Example 744, Example 745, Example 746, Example 747, Example 748, Example 749, Example 750, Example 751, Example 752, Example 753, Example 754, Example 755 or Example 756.

50. A pharmaceutical composition comprising a compound of any one of claims 1-49 or a pharmaceutically acceptable salt, stereoisomer, tautomer or prodrug thereof, together with a pharmaceutically acceptable excipient.

51. A method of treating a disease that can be affected by EGFR modulation, comprises administrating a subject in need thereof an effective amount of a compound of any one of claims 1-49 or a pharmaceutically acceptable salt, stereoisomer, tautomer or prodrug thereof.

52. The method of claim 51, wherein the disease is selected from cancer, preferred pancreatic cancer, breast cancer, glioblastoma multiforme, head and neck cancer, or non-small cell lung cancer.

53. Use of a compound of any one of claims 1-49 or a pharmaceutically acceptable salt, stereoisomer, tautomer or prodrug thereof in the preparation of a medicament for treating a disease that can be affected by EGFR modulation.

54. The use of claim 53, wherein the disease is cancer, preferred pancreatic cancer, breast cancer, glioblastoma multiforme, head and neck cancer, or non-small cell lung cancer.
